(19) United States
(12) Patent Application Publication
(10) Pub. No.: US 2003/0216558 A1
(43) Pub. Date: Nov. 20, 2003

(54) Novel compositions and methods for cancer

(76) Inventors: David W. Morris, Davis, CA (US); Eric K. Engelhard, Davis, CA (US)

Correspondence Address: Shantanu Basu, Morrison & Foerster LLP, 755 Page Mill Road, Palo Alto, CA, 94304-1018, US

(21) Appl. No.: 10/034,650

(22) Filed: Dec. 20, 2001

Related U.S. Application Data
(63) Continuation-in-part of application No. 09/747,377, filed on Dec. 22, 2000.
(63) Continuation-in-part of application No. 09/798,586, filed on Mar. 2, 2001.

(51) Int. Cl. C07H 21/02; C07H 21/04

(52) U.S. Cl. 536/23.1

(57) ABSTRACT

The present invention relates to novel sequences for use in diagnosis and treatment of carcinomas, especially lymphoma carcinomas. In addition, the present invention describes the use of novel compositions for use in screening methods.

The present application is a continuing application of U.S. Ser. Nos. 09/747,377, filed Dec. 22, 2000 and 09/798,586, filed Mar. 2, 2001, both of which are expressly incorporated herein by reference.

### FIELD OF THE INVENTION

The present invention relates to novel sequences for use in diagnosis and treatment of cancer, especially carcinomas, as well as the use of the novel compositions in screening methods.

### BACKGROUND OF THE INVENTION

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways, including promoter insertion, enhancer insertion, and/or truncation of a protooncogene or tumor suppressor gene. The analysis of sequences at or near the insertion sites led to the identification of a number of new protooncogenes.

With respect to lymphoma and leukemia, murine leukemia retrovirus (MuLV), such as SL3-3 or Akv, is a potent inducer of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al. J. Virology 67:7118 (1993); Joosten et al., Virology 268:308 (2000); and Li et al., Nature Genetics 23:348 (1999); all of which are expressly incorporated by reference herein.

Accordingly, it is an object of the invention to provide sequences involved in cancer and in particular in oncogenesis.

### SUMMARY OF THE INVENTION

In accordance with the objects outlined above, the present invention provides methods for screening for compositions which modulate carcinomas, especially lymphoma and leukemia. Also provided herein are methods of inhibiting proliferation of a cell, preferably a lymphoma cell. Methods of treatment of carcinomas, including diagnosis, are also provided herein.

In one aspect, a method of screening drug candidates comprises providing a cell that expresses a carcinoma associated (CA) gene or fragments thereof. Preferred embodiments of CA genes are genes which are differentially expressed in cancer cells, preferably lymphatic, breast, prostate or epithelial cells, compared to other cells. Preferred embodiments of CA genes used in the methods herein include, but are not limited to the nucleic acids selected from Tables 1-10. The method further includes adding a drug candidate to the cell and determining the effect of the drug candidate on the expression of the CA gene.

In one embodiment, the method of screening drug candidates includes comparing the level of expression in the absence of the drug candidate to the level of expression in the presence of the drug candidate.

Also provided herein is a method of screening for a bioactive agent capable of binding to a CA protein (CAP), the method comprising combining the CAP and a candidate bioactive agent, and determining the binding of the candidate agent to the CAP.

Further provided herein is a method for screening for a bioactive agent capable of modulating the activity of a CAP. In one embodiment, the method comprises combining the CAP and a candidate bioactive agent, and determining the effect of the candidate agent on the bioactivity of the CAP.

Also provided is a method of evaluating the effect of a candidate carcinoma drug comprising administering the drug to a patient and removing a cell sample from the patient. The expression profile of the cell is then determined. This method may further comprise comparing the expression profile of the patient to an expression profile of a heathy individual.

In a further aspect, a method for inhibiting the activity of an CA protein is provided. In one embodiment, the method comprises administering to a patient an inhibitor of a CA protein preferably selected from the group consisting of the sequences outlined in Tables 1-10 or their complements.

A method of neutralizing the effect of a CA protein, preferably a protein encoded by a nucleic acid selected from the group of sequences outlined in Tables 1-10, is also provided. Preferably, the method comprises contacting an agent specific for said protein with said protein in an amount sufficient to effect neutralization.

Moreover, provided herein is a biochip comprising a nucleic acid segment which encodes a CA protein, preferably selected from the sequences outlined in Tables 1-10.

Also provided herein is a method for diagnosing or determining the propensity to carcinomas, especially lymphoma or leukemia by sequencing at least one carcinoma or lymphoma gene of an individual. In yet another aspect of the invention, a method is provided for determining carcinoma including lymphoma and leukemia gene copy number in an individual.

Novel sequences are also provided herein. Other aspects of the invention will become apparent to the skilled artisan by the following description of the invention.

### DETAILED DESCRIPTION OF THE INVENTION

The present invention is directed to a number of sequences associated with carcinomas, especially lymphoma, breast cancer or prostate cancer. The relatively tight linkage between clonally-integrated proviruses and protooncogenes forms “provirus tagging”, in which slow-transforming retroviruses that act by an insertion mutation mechanism are used to isolate protooncogenes. In some models, uninfected animals have low cancer rates, and infected animals have high cancer rates. It is known that many of the retroviruses involved do not carry transduced host protooncogenes or pathogenic trans-acting viral genes, and thus the cancer incidence must therefor be a direct consequence of proviral integration effects into host protooncogenes. Since proviral integration is random, rare integrants will “activate” host protooncogenes that provide a selective growth advantage, and these rare events result in new proviruses at clonal stoichiometries in tumors.

The use of oncogenic retroviruses, whose sequences insert into the genome of the host organism resulting in carcinoma, allows the identification of host sequences involved in carcinoma. These sequences may then be used in a number of different ways, including diagnosis, prognosis, screening for modulators (including both agonists and antagonists), antibody generation (for immunotherapy and imaging), etc. However, as will be appreciated by those in the art, oncogenes that are identified in one type of cancer such as lymphoma or leukemia have a strong likelihood of being involved in other types of cancers as well. Thus, while the sequences outlined herein are initially identified as correlated with lymphoma, they can also be found in other types of cancers as well, outlined below.

Accordingly, the present invention provides nucleic acid and protein sequences that are associated with carcinoma, herein termed “carcinoma associated” or “CA” sequences. In a preferred embodiment, the present invention provides nucleic acid and protein sequences that are associated with carcinomas which originate in lymphatic tissue, herein termed “lymphoma associated”, “leukemia associated” or “LA” sequences.

Suitable cancers which can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); cancers of the lymphomas (hodgkin's disease and non-hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Branchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In addition, the genes may be involved in other diseases, such as but not limited to diseases associated with aging or neurodegenerative diseases.

Association in this context means that the nucleotide or protein sequences are either differentially expressed, activated, inactivated or altered in carcinomas as compared to normal tissue. As outlined below, CA sequences include those that are up-regulated (i.e. expressed at a higher level), as well as those that are down-regulated (i.e. expressed at a lower level), in carcinomas. CA sequences also include sequences which have been altered (i.e., truncated sequences or sequences with substitutions, deletions or insertions, including point mutations) and show either the same expression profile or an altered profile. In a preferred embodiment, the CA sequences are from humans; however, as will be appreciated by those in the art, CA sequences from other organisms may be useful in animal models of disease and drug evaluation; thus, other CA sequences are provided, from vertebrates, including mammals, including rodents (rats, mice, hamsters, guinea pigs, etc.), primates, farm animals (including sheep, goats, pigs, cows, horses, etc). In some cases, prokaryotic CA sequences may be useful. CA sequences from other organisms may be obtained using the techniques outlined below.

CA sequences can include both nucleic acid and amino acid sequences. In a preferred embodiment, the CA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an CA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the CA sequences are nucleic acids. As will be appreciated by those in the art and is more fully outlined below, CA sequences are useful in a variety of applications, including diagnostic applications, which will detect naturally occurring nucleic acids, as well as screening applications; for example, biochips comprising nucleic acid probes to the CA sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below (for example in antisense applications or when a candidate agent is a nucleic acid), nucleic acid analogs may be used that have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand “Watson” also defines the sequence of the other strand “Crick”; thus the sequences described herein also includes the complement of the sequence. The nucleic acid may be DNA, both genomic and cDNA, RNA or a hybrid, where the nucleic acid contains any combination of deoxyriboand ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are is referred to herein as a nucleoside.

An CA sequence can be initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

The CA sequences of the invention were initially identified as described herein; basically, infection of mice with murine leukemia viruses (MLV) resulted in lymphoma, although many of these sequences will also be involved in other cancers as is generally outlined herein.

The CA sequences outlined herein comprise the insertion sites for the virus. In general, the retrovirus can cause carcinomas in three basic ways: first of all, by inserting upstream of a normally silent host gene and activating it (e.g. promoter insertion); secondly, by truncating a host gene that leads to oncogenesis; or by enhancing the transcription of a neighboring gene. For example, retrovirus enhancers, including SL3-3, are known to act on genes up to approximately 200 kilobases of the insertion site.

In a preferred embodiment, CA sequences are those that are up-regulated in carcinomas; that is, the expression of these genes is higher in carcinoma tissue as compared to normal tissue of the same differentiation stage. “Up-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are down-regulated in carcinomas; that is, the expression of these genes is lower in carcinoma tissue as compared to normal I tissue of the same differentiation stage. “Down-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are altered but show either the same expression profile or an altered profile as compared to normal lymphoid tissue of the same differentiation stage. “Altered CA sequences” as used herein refers to sequences which are truncated, contain insertions or contain point mutations.

CA proteins of the present invention may be classified as secreted proteins, transmembrane proteins or intracellular proteins.

In a preferred embodiment the CA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the CA sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor, etc.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

CA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the CA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. CA proteins that are secreted proteins are particularly preferred in the present invention as they serve as good targets for diagnostic markers, for example for blood tests.

An CA sequence is initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

As used herein, a nucleic acid is a “CA nucleic acid” if the overall homology of the nucleic acid sequence to one of the nucleic acids of Tables 1-10 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences which are used to determine sequence identity or similarity are selected from those of the nucleic acids of Tables 1-10. In another embodiment, the sequences are naturally occurring allelic variants of the sequences of the nucleic acids of Tables 1-10. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acids of Tables 1-10. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of Tables 1-10, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, for example, nucleic acids which hybridize under high stringency to the nucleic acids identified in the figures, or their complements, are considered CA sequences. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

In addition, the CA nucleic acid sequences of the invention are fragments of larger genes, i.e. they are nucleic acid segments. Alternatively, the CA nucleic acid sequences can serve as indicators of oncogene position, for example, the CA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the CA genes can be obtained, using techniques well known in the art for cloning either longer sequences or the full length sequences; see Maniatis et al., and Ausubel, et al., supra, hereby expressly incorporated by reference. In general, this is done using PCR, for example, kinetic PCR.

Once the CA nucleic acid is identified, it can be cloned and, if necessary, its constituent parts recombined to form the entire CA nucleic acid. Once isolated from its natural source, e.g., contained within a plasmid or other vector or excised therefrom as a linear nucleic acid segment, the recombinant CA nucleic acid can be further used as a probe to identify and isolate other CA nucleic acids, for example additional coding regions. It can also be used as a “precursor” nucleic acid to make modified or variant CA nucleic acids and proteins.

The CA nucleic acids of the present invention are used in several ways. In a first embodiment, nucleic acid probes to the CA nucleic acids are made and attached to biochips to be used in screening and diagnostic methods, as outlined below, or for administration, for example for gene therapy and/or antisense applications. Alternatively, the CA nucleic acids that include coding regions of CA proteins can be put into expression vectors for the expression of CA proteins, again either for screening purposes or for administration to a patient.

In a preferred embodiment, nucleic acid probes to CA nucleic acids (both the nucleic acid sequences outlined in the figures and/or the complements thereof) are made. The nucleic acid probes attached to the biochip are designed to be substantially complementary to the CA nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, resins, silica or silica-based materials including silicon and modified silicon, carbon, metals, inorganic glasses, etc. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affymetrix GeneChip technology.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, and an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

In a preferred embodiment, CA nucleic acids encoding CA proteins are used to make a variety of expression vectors to express CA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the CA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the CA protein; for example, transcriptional and translational regulatory nucleic acid sequences from Bacillus are preferably used to express the CA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The CA proteins of the present invention are produced by culturing a host cell transformed with an expression vector containing nucleic acid encoding an CA protein, under the appropriate conditions to induce or cause expression of the CA protein. The conditions appropriate for CA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the CA proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, CA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the CA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, CA proteins are produced in insect cells. Expression vectors for the transformation of insect cells, and in particular, baculovirus-based expression vectors, are well known in the art.

In a preferred embodiment, CA protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The CA protein may also be made as a fusion protein, using techniques well known in the art. Thus, for example, for the creation of monoclonal antibodies. If the desired epitope is small, the CA protein may be fused to a carrier protein to form an immunogen. Alternatively, the CA protein may be made as a fusion protein to increase expression, or for other reasons. For example, when the CA protein is an CA peptide, the nucleic acid encoding the peptide may be linked to other nucleic acid for expression purposes.

In one embodiment, the CA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the CA nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides CA protein sequences. An CA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the CA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of CA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

CA proteins of the present invention may be shorter or longer than the wild type amino acid sequences. Thus, in a preferred embodiment, included within the definition of CA proteins are portions or fragments of the wild type sequences herein. In addition, as outlined above, the CA nucleic acids of the invention may be used to obtain additional coding regions, and thus additional protein sequence, using techniques known in the art.

In a preferred embodiment, the CA proteins are derivative or variant CA proteins as compared to the wild-type sequence. That is, as outlined more fully below, the derivative CA peptide will contain at least one amino acid substitution, deletion or insertion, with amino acid substitutions being particularly preferred. The amino acid substitution, insertion or deletion may occur at any residue within the CA peptide.

Also included in an embodiment of CA proteins of the present invention are amino acid sequence variants. These variants fall into one or more of three classes: substitutional, insertional or deletional variants. These variants ordinarily are prepared by site specific mutagenesis of nucleotides in the DNA encoding the CA protein, using cassette or PCR mutagenesis or other techniques well known in the art, to produce DNA encoding the variant, and thereafter expressing the DNA in recombinant cell culture as outlined above. However, variant CA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the CA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed CA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of CA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the CA protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart 1. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CA proteins as needed. Alternatively, the variant may be designed such that the biological activity of the CA protein is altered. For example, glycosylation sites may be altered or removed, dominant negative mutations created, etc.

Covalent modifications of CA polypeptides are included within the scope of this invention, for example for use in screening. One type of covalent modification includes reacting targeted amino acid residues of an CA polypeptide with an organic derivatizing agent that is capable of reacting with selected side chains or the N-or C-terminal residues of an CA polypeptide. Derivatization with bifunctional agents is useful, for instance, for crosslinking CA polypeptides to a water-insoluble support matrix or surface for use in the method for purifying anti-CA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the a-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the CA polypeptide included within the scope of this invention comprises altering the native glycosylation pattern of the polypeptide. “Altering the native glycosylation pattern” is intended for purposes herein to mean deleting one or more carbohydrate moieties found in native sequence CA polypeptide, and/or adding one or more glycosylation sites that are not present in the native sequence CA polypeptide.

Addition of glycosylation sites to CA polypeptides may be accomplished by altering the amino acid sequence thereof. The alteration may be made, for example, by the addition of, or substitution by, one or more serine or threonine residues to the native sequence CA polypeptide (for o-linked glycosylation sites). The CA amino acid sequence may optionally be altered through changes at the DNA level, particularly by mutating the DNA encoding the CA polypeptide at preselected bases such that codons are generated that will translate into the desired amino acids.

Another means of increasing the number of carbohydrate moieties on the CA polypeptide is by chemical or enzymatic coupling of glycosides to the polypeptide. Such methods are described in the art, e.g., in WO 87/05330 published Sep. 1987, and in Aplin and Wriston, LA Crit. Rev. Biochem., pp. 259-306 (1981).

Removal of carbohydrate moieties present on the CA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of CA comprises linking the CA polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

CA polypeptides of the present invention may also be modified in a way to form chimeric molecules comprising an CA polypeptide fused to another, heterologous polypeptide or amino acid sequence. In one embodiment, such a chimeric molecule comprises a fusion of an CA polypeptide with a tag polypeptide which provides an epitope to which an anti-tag antibody can selectively bind. The epitope tag is generally placed at the amino-or carboxyl-terminus of the CA polypeptide, although internal fusions may also be tolerated in some instances. The presence of such epitope-tagged forms of an CA polypeptide can be detected using an antibody against the tag polypeptide. Also, provision of the epitope tag enables the CA polypeptide to be readily purified by affinity purification using an anti-tag antibody or another type of affinity matrix that binds to the epitope tag. In an alternative embodiment, the chimeric molecule may comprise a fusion of an CA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of CA protein in one embodiment are other CA proteins of the CA family, and CA proteins from other organisms, which are cloned and expressed as outlined below. Thus, probe or degenerate polymerase chain reaction (PCR) primer sequences may be used to find other related CA proteins from humans or other organisms. As will be appreciated by those in the art, particularly useful probe and/or PCR primer sequences include the unique areas of the CA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, CA proteins can be made that are longer than those encoded by the nucleic acids of the figures, for example, by the elucidation of additional sequences, the addition of epitope or purification tags, the addition of other fusion sequences, etc.

CA proteins may also be identified as being encoded by CA nucleic acids. Thus, CA proteins are encoded by nucleic acids that will hybridize to the sequences of the sequence listings, or their complements, as outlined herein.

In a preferred embodiment, the invention provides CA antibodies. In a preferred embodiment, when the CA protein is to be used to generate antibodies, for example for immunotherapy, the CA protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller CA protein will be able to bind to the full length protein. In a preferred embodiment, the epitope is unique; that is, antibodies generated to a unique epitope show little or no cross-reactivity.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-10, or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a protein encoded by a nucleic acid of Tables 1-10, or a fragment thereof, the other one is for any other antigen, and preferably for a cell-surface protein or receptor or receptor subunit, preferably one that is tumor specific.

In a preferred embodiment, the antibodies to CA are capable of reducing or eliminating the biological function of CA, as is described below. That is, the addition of anti-CA antibodies (either polyclonal or preferably monoclonal) to CA (or cells containing CA) may reduce or eliminate the CA activity. Generally, at least a 25% decrease in activity is preferred, with at least about 50% being particularly preferred and about a 95-100% decrease being especially preferred.

In a preferred embodiment the antibodies to the CA proteins are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Similarly, human can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of a carcinoma with an antibody raised against an CA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against CA proteins that are secreted proteins as described above. Without being bound by theory, antibodies used for treatment, bind and prevent the secreted protein from binding to its receptor, thereby inactivating the secreted CA protein.

In another preferred embodiment, the CA protein to which antibodies are raised is a transmembrane protein. Without being bound by theory, antibodies used for treatment, bind the extracellular domain of the CA protein and prevent it from binding to other proteins, such as circulating ligands or cell-associated molecules. The antibody may cause down-regulation of the transmembrane CA protein. As will be appreciated by one of ordinary skill in the art, the antibody may be a competitive, non-competitive or uncompetitive inhibitor of protein binding to the extracellular domain of the CA protein. The antibody is also an antagonist of the CA protein. Further, the antibody prevents activation of the transmembrane CA protein. In one aspect, when the antibody prevents the binding of other molecules to the CA protein, the antibody prevents growth of the cell. The antibody may also sensitize the cell to cytotoxic agents, including, but not limited to TNF-α, TNF-β, IL-1, INF-y and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, carcinomas may be treated by administering to a patient antibodies directed against the transmembrane CA protein.

In another preferred embodiment, the antibody is conjugated to a therapeutic moiety. In one aspect the therapeutic moiety is a small molecule that modulates the activity of the CA protein. In another aspect the therapeutic moiety modulates the activity of molecules associated with or in close proximity to the CA protein. The therapeutic moiety may inhibit enzymatic activity such as protease or protein kinase activity associated with carcinoma.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with carcinomas, including lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against CA proteins, or binding of a radionuclide to a chelating agent that has been covalently attached to the antibody. Targeting the therapeutic moiety to transmembrane CA proteins not only serves to increase the local concentration of therapeutic moiety in the carcinoma of interest, i.e., lymphoma, but also serves to reduce deleterious side effects that may be associated with the therapeutic moiety.

In another preferred embodiment, the CA protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the CA protein can be targeted within a cell, i.e., the nucleus, an antibody thereto contains a signal for that target localization, i.e., a nuclear localization signal.

The CA antibodies of the invention specifically bind to CA proteins. By “specifically bind” herein is meant that the antibodies bind to the protein with a binding constant in the range of at least 10⁻⁴-10⁻⁶ M⁻¹, with a preferred range being 10⁻⁷⁻10⁻⁹ M⁻¹.

In a preferred embodiment, the CA protein is purified or isolated after expression. CA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CA protein may be purified using a standard anti-CA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the CA protein. In some instances no purification will be necessary.

Once expressed and purified if necessary, the CA proteins and nucleic acids are useful in a number of applications.

In one aspect, the expression levels of genes are determined for different cellular states in the carcinoma phenotype; that is, the expression levels of genes in normal tissue and in carcinoma tissue (and in some cases, for varying severities of lymphoma that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or carcinoma tissue.

“Differential expression,” or grammatical equivalents as used herein, refers to both qualitative as well as quantitative differences in the genes temporal and/or cellular expression patterns within and among the cells. Thus, a differentially expressed gene can qualitatively have its expression altered, including an activation or inactivation, in, for example, normal versus carcinoma tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip® expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the CA protein and standard immunoassays (ELISAs, etc.) or other techniques, including mass spectroscopy assays, 2D gel electrophoresis assays, etc. Thus, the proteins corresponding to CA genes, i.e. those identified as being important in a particular carcinoma phenotype, i.e., lymphoma, can be evaluated in a diagnostic test specific for that carcinoma.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the CA nucleic acid probes may be attached to biochips as outlined herein for the detection and quantification of CA sequences in a particular cell. The assays are done as is known in the art. As will be appreciated by those in the art, any number of different CA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect CA sequences that are up-regulated or down-regulated in carcinomas as compared to normal tissue. In instances where the CA sequence has been altered but shows the same expression profile or an altered expression profile, the protein will be detected as outlined herein.

In a preferred embodiment nucleic acids encoding the CA protein are detected. Although DNA or RNA encoding the CA protein may be detected, of particular interest are methods wherein the mRNA encoding a CA protein is detected. The presence of mRNA in a sample is an indication that the CA gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a CA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The CA proteins, antibodies, nucleic acids, modified proteins and cells containing CA sequences are used in diagnostic assays. This can be done on an individual gene or corresponding polypeptide level, or as sets of assays.

As described and defined herein, CA proteins find use as markers of carcinomas, including lymphomas such as, but not limited to, Hodgkin's and non-Hodgkin lymphoma. Detection of these proteins in putative carcinoma tissue or patients allows for a determination or diagnosis of the type of carcinoma. Numerous methods known to those of ordinary skill in the art find use in detecting carcinomas. In one embodiment, antibodies are used to detect CA proteins. A preferred method separates proteins from a sample or patient by electrophoresis on a gel (typically a denaturing and reducing protein gel, but may be any other type of gel including isoelectric focusing gels and the like). Following separation of proteins, the CA protein is detected by immunoblotting with antibodies raised against the CA protein. Methods of immunoblotting are well known to those of ordinary skill in the art.

In another preferred method, antibodies to the CA protein find use in in situ imaging techniques. In this method cells are contacted with from one to many antibodies to the CA protein(s). Following washing to remove non-specific antibody binding, the presence of the antibody or antibodies is detected. In one embodiment the antibody is detected by incubating with a secondary antibody that contains a detectable label. In another method the primary antibody to the CA protein(s) contains a detectable label. In another preferred embodiment each one of multiple primary antibodies contains a distinct and detectable label. This method finds particular use in simultaneous screening for a plurality of CA proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing carcinomas from blood samples. As previously described, certain CA proteins are secreted/circulating molecules. Blood samples, therefore, are useful as samples to be probed or tested for the presence of secreted CA proteins. Antibodies can be used to detect the CA proteins by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled CA nucleic acid probes to tissue arrays is done. For example, arrays of tissue samples, including CA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis.

In a preferred embodiment, the CA proteins, antibodies, nucleic acids, modified proteins and cells containing CA sequences are used in prognosis assays. As above, gene expression profiles can be generated that correlate to carcinoma, especially lymphoma, severity, in terms of long term prognosis. Again, this may be done on either a protein or gene level, with the use of genes being preferred. As above, the CA probes are attached to biochips for the detection and quantification of CA sequences in a tissue or patient. The assays proceed as outlined for diagnosis.

In a preferred embodiment, any of the CA sequences as described herein are used in drug screening assays. The CA proteins, antibodies, nucleic acids, modified proteins and cells containing CA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In a preferred embodiment, the CA proteins, antibodies, nucleic acids, modified proteins and cells containing the native or modified CA proteins are used in screening assays. That is, the present invention provides novel methods for screening for compositions which modulate the carcinoma phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the CA genes herein, a variety of assays to evaluate the effects of agents on gene expression may be executed. In a preferred embodiment, assays may be run on an individual gene or protein level. That is, having identified a particular gene as aberrantly regulated in carcinoma, candidate bioactive agents may be screened to modulate the genes response. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the CA sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the CA protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the CA nucleic acid probes are attached to biochips as outlined herein for the detection and quantification of CA sequences in a particular cell. The assays are further described below.

Generally, in a preferred embodiment, a candidate bioactive agent is added to the cells prior to analysis. Moreover, screens are provided to identify a candidate bioactive agent which modulates a particular type of carcinoma, modulates CA proteins, binds to a CA protein, or interferes between the binding of a CA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the carcinoma phenotype, binding to and/or modulating the bioactivity of an CA protein, or the expression of a CA sequence, including both nucleic acid sequences and protein sequences. In a particularly preferred embodiment, the candidate agent suppresses a CA phenotype, for example to a normal tissue fingerprint. Similarly, the candidate agent preferably suppresses a severe CA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of an CA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In assays for altering the expression profile of one or more CA genes, after the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with a label as defined herein, with biotin-FITC or PE, cy3 and cy5 being particularly preferred.

In a preferred embodiment, the target sequence is labeled with, for example, a fluorescent, chemiluminescent, chemical, or radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin.

For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to alter the expression of the genes individually. That is, screening for modulation of regulation of expression of a single gene can be done. Thus, for example, particularly in the case of target genes whose presence or absence is unique between two states, screening is done for modulators of the target gene expression.

In addition, screens can be done for novel genes that are induced in response to a candidate agent. After identifying a candidate agent based upon its ability to suppress a CA expression pattern leading to a normal expression pattern, or modulate a single CA gene expression profile so as to mimic the expression of the gene from normal tissue, a screen as described above can be performed to identify genes that are specifically modulated in response to the agent. Comparing expression profiles between normal tissue and agent treated CA tissue reveals genes that are not expressed in normal tissue or CA tissue, but are expressed in agent treated tissue. These agent specific sequences can be identified and used by any of the methods described herein for CA genes or proteins. In particular these sequences and the proteins they encode find use in marking or identifying agent treated cells. In addition, antibodies can be raised against the agent induced proteins and used to target novel therapeutics to the treated CA tissue sample.

Thus, in one embodiment, a candidate agent is administered to a population of CA cells, that thus has an associated CA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, CA tissue may be screened for agents that reduce or suppress the CA phenotype. A change in at least one gene of the expression profile indicates that the agent has an effect on CA activity. By defining such a signature for the CA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “CA proteins” or an “CAP”. The CAP may be a fragment, or alternatively, be the full length protein to the fragment encoded by the nucleic acids of Tables 1-10. Preferably, the CAP is a fragment. In another embodiment, the sequences are sequence variants as further described herein.

Preferably, the CAP is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the c-terminus of the fragment is kept as a free acid and the n-terminus is a free amine to aid in coupling, i.e., to cysteine.

In one embodiment the CA proteins are conjugated to an immunogenic agent as discussed herein. In one embodiment the CA protein is conjugated to BSA.

In a preferred embodiment, screening is done to alter the biological function of the expression product of the CA gene. Again, having identified the importance of a gene in a particular state, screening for agents that bind and/or modulate the biological activity of the gene product can be run as is more fully outlined below.

In a preferred embodiment, screens are designed to first find candidate agents that can bind to CA proteins, and then these agents may be used in assays that evaluate the ability of the candidate agent to modulate the CAP activity and the carcinoma phenotype. Thus, as will be appreciated by those in the art, there are a number of different assays which may be run; binding assays and activity assays.

In a preferred embodiment, binding assays are done. In general, purified or isolated gene product is used; that is, the gene products of one or more CA nucleic acids are made. In general, this is done as is known in the art. For example, antibodies are generated to the protein gene products, and standard immunoassays are run to determine the amount of protein present. Alternatively, cells comprising the CA proteins can be used in the assays.

Thus, in a preferred embodiment, the methods comprise combining a CA protein and a candidate bioactive agent, and determining the binding of the candidate agent to the CA protein. Preferred embodiments utilize the human or mouse CA protein, although other mammalian proteins may also be used, for example for the development of animal models of human disease. In some embodiments, as outlined herein, variant or derivative CA proteins may be used.

Generally, in a preferred embodiment of the methods herein, the CA protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the CA protein is bound to the support, and a candidate bioactive agent is added to the assay. Alternatively, the candidate agent is bound to the support and the CA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the CA protein may be done in a number of ways. In a preferred embodiment, the candidate bioactive agent is labeled, and binding determined directly. For example, this may be done by attaching all or a portion of the CA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or is indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. CA protein), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the CA protein and thus is capable of binding to, and potentially modulating, the activity of the CA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the CA protein with a higher affinity. Thus, if the candidate bioactive agent is labeled, the presence of the label on the support, coupled with a lack of competitor binding, may indicate that the candidate agent is capable of binding to the CA protein.

In a preferred embodiment, the methods comprise differential screening to identity bioactive agents that are capable of modulating the activity of the CA proteins. In this embodiment, the methods comprise combining a CA protein and a competitor in a first sample. A second sample comprises a candidate bioactive agent, a CA protein and a competitor. The binding of the competitor is determined for both samples, and a change, or difference in binding between the two samples indicates the presence of an agent capable of binding to the CA protein and potentially modulating its activity. That is, if the binding of the competitor is different in the second sample relative to the first sample, the agent is capable of binding to the CA protein.

Alternatively, a preferred embodiment utilizes differential screening to identify drug candidates that bind to the native CA protein, but cannot bind to modified CA proteins. The structure of the CA protein may be modeled, and used in rational drug design to synthesize agents that interact with that site. Drug candidates that affect CA bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of CA proteins may also be done. In a preferred embodiment, methods for screening for a bioactive agent capable of modulating the activity of CA proteins comprise the steps of adding a candidate bioactive agent to a sample of CA proteins, as above, and determining an alteration in the biological activity of CA proteins. “Modulating the activity of an CA protein” includes an increase in activity, a decrease in activity, or a change in the type or kind of activity present. Thus, in this embodiment, the candidate agent should both bind to CA proteins (although this may not be necessary), and alter its biological or biochemical activity as defined herein. The methods include both in vitro screening methods, as are generally outlined above, and in vivo screening of cells for alterations in the presence, distribution, activity or amount of CA proteins.

Thus, in this embodiment, the methods comprise combining a CA sample and a candidate bioactive agent, and evaluating the effect on CA activity. By “CA activity” or grammatical equivalents herein is meant one of the CA protein's biological activities, including, but not limited to, its role in tumorigenesis, including cell division, preferably in lymphatic tissue, cell proliferation, tumor growth and transformation of cells. In one embodiment, CA activity includes activation of or by a protein encoded by a nucleic acid of Tables 1-10. An inhibitor of CA activity is the inhibition of any one or more CA activities.

In a preferred embodiment, the activity of the CA protein is increased; in another preferred embodiment, the activity of the CA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a CA protein. The methods comprise adding a candidate bioactive agent, as defined above, to a cell comprising CA proteins. Preferred cell types include almost any cell. The cells contain a recombinant nucleic acid that encodes a CA protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the CA protein.

In one embodiment, a method of inhibiting carcinoma cancer cell division, is provided. The method comprises administration of a carcinoma cancer inhibitor.

In a preferred embodiment, a method of inhibiting lymphoma carcinoma cell division is provided comprising administration of a lymphoma carcinoma inhibitor.

In another embodiment, a method of inhibiting tumor growth is provided. The method comprises administration of a carcinoma cancer inhibitor. In a particularly preferred embodiment, a method of inhibiting tumor growth in lymphatic tissue is provided comprising administration of a lymphoma inhibitor.

In a further embodiment, methods of treating cells or individuals with cancer are provided. The method comprises administration of a carcinoma cancer inhibitor. Preferably, the carcinoma is a lymphoma carcinoma.

In one embodiment, a carcinoma cancer inhibitor is an antibody as discussed above. In another embodiment, the carcinoma cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for carcinoma cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., Bio Techniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various CA sequences are important in carcinomas. Accordingly, disorders based on mutant or variant CA genes may be determined. In one embodiment, the invention provides methods for identifying cells containing variant CA genes comprising determining all or part of the sequence of at least one endogenous CA genes in a cell. As will be appreciated by those in the art, this may be done using any number of sequencing techniques. In a preferred embodiment, the invention provides methods of identifying the CA genotype of an individual comprising determining all or part of the sequence of at least one CA gene of the individual. This is generally done in at least one tissue of the individual, and may include the evaluation of a number of tissues or different samples of the same tissue. The method may include comparing the sequence of the sequenced CA gene to a known CA gene, i.e., a wild-type gene. As will be appreciated by those in the art, alterations in the sequence of some oncogenes can be an indication of either the presence of the disease, or propensity to develop the disease, or prognosis evaluations.

The sequence of all or part of the CA gene can then be compared to the sequence of a known CA gene to determine if any differences exist. This can be done using any number of known homology programs, such as Bestfit, etc. In a preferred embodiment, the presence of a difference in the sequence between the CA gene of the patient and the known CA gene is indicative of a disease state or a propensity for a disease state, as outlined herein.

In a preferred embodiment, the CA genes are used as probes to determine the number of copies of the CA gene in the genome. For example, some cancers exhibit chromosomal deletions or insertions, resulting in an alteration in the copy number of a gene.

In another preferred embodiment CA genes are used as probes to determine the chromosomal location of the CA genes. Information such as chromosomal location finds use in providing a diagnosis or prognosis in particular when chromosomal abnormalities such as translocations, and the like are identified in CA gene loci.

Thus, in one embodiment, methods of modulating CA in cells or organisms are provided. In one embodiment, the methods comprise administering to a cell an anti-CA antibody that reduces or eliminates the biological activity of an endogenous CA protein. Alternatively, the methods comprise administering to a cell or organism a recombinant nucleic acid encoding a CA protein. As will be appreciated by those in the art, this may be accomplished in any number of ways. In a preferred embodiment, for example when the CA sequence is down-regulated in carcinoma, the activity of the CA gene is increased by increasing the amount of CA in the cell, for example by overexpressing the endogenous CA or by administering a gene encoding the CA sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the CA sequence is up-regulated in carcinoma, the activity of the endogenous CA gene is decreased, for example by the administration of a CA antisense nucleic acid.

In one embodiment, the CA proteins of the present invention may be used to generate polyclonal and monoclonal antibodies to CA proteins, which are useful as described herein. Similarly, the CA proteins can be coupled, using standard technology, to affinity chromatography columns. These columns may then be used to purify CA antibodies. In a preferred embodiment, the antibodies are generated to epitopes unique to a CA protein; that is, the antibodies show little or no cross-reactivity to other proteins. These antibodies find use in a number of applications. For example, the CA antibodies may be coupled to standard affinity chromatography columns and used to purify CA proteins. The antibodies may also be used as blocking polypeptides, as outlined above, since they will specifically bind to the CA protein.

In one embodiment, a therapeutically effective dose of a CA or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for CA degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the CA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the CA proteins and modulators may be directly applied as a solution or spray.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, CA proteins and modulators are administered as therapeutic agents, and can be formulated as outlined above. Similarly, CA genes (including both the full-length sequence, partial sequences, or regulatory sequences of the CA coding regions) can be administered in gene therapy applications, as is known in the art. These CA genes can include antisense applications, either as gene therapy (i.e. for incorporation into the genome) or as antisense compositions, as will be appreciated by those in the art.

In a preferred embodiment, CA genes are administered as DNA vaccines, either single genes or combinations of CA genes. Naked DNA vaccines are generally known in the art. Brower, Nature Biotechnology, 16:1304-1305 (1998).

In one embodiment, CA genes of the present invention are used as DNA vaccines. Methods for the use of genes as DNA vaccines are well known to one of ordinary skill in the art, and include placing a CA gene or portion of a CA gene under the control of a promoter for expression in a patient with carcinoma. The CA gene used for DNA vaccines can encode full-length CA proteins, but more preferably encodes portions of the CA proteins including peptides derived from the CA protein. In a preferred embodiment a patient is immunized with a DNA vaccine comprising a plurality of nucleotide sequences derived from a CA gene. Similarly, it is possible to immunize a patient with a plurality of CA genes or portions thereof as defined herein. Without being bound by theory, expression of the polypeptide encoded by the DNA vaccine, cytotoxic T-cells, helper T-cells and antibodies are induced which recognize and destroy or eliminate cells expressing CA proteins.

In a preferred embodiment, the DNA vaccines include a gene encoding an adjuvant molecule with the DNA vaccine. Such adjuvant molecules include cytokines that increase the immunogenic response to the CA polypeptide encoded by the DNA vaccine. Additional or alternative adjuvants are known to those of ordinary skill in the art and find use in the invention.

In another preferred embodiment CA genes find use in generating animal models of carcinomas, particularly lymphoma carcinomas. As is appreciated by one of ordinary skill in the art, when the CA to gene identified is repressed or diminished in CA tissue, gene therapy technology wherein antisense RNA directed to the CA gene will also diminish or repress expression of the gene. An animal generated as such serves as an animal model of CA that finds use in screening bioactive drug candidates. Similarly, gene knockout technology, for example as a result of homologous recombination with an appropriate gene targeting vector, will result in the absence of the CA protein. When desired, tissue-specific expression or knockout of the CA protein may be necessary.

It is also possible that the CA protein is overexpressed in carcinoma. As such, transgenic animals can be generated that overexpress the CA protein. Depending on the desired expression level, promoters of various strengths can be employed to express the transgene. Also, the number of copies of the integrated transgene can be determined and compared for a determination of the expression level of the transgene. Animals generated by such methods find use as animal models of CA and are additionally useful in screening for bioactive molecules to treat carcinoma.

The CA nucleic acid sequences of the invention are depicted in Tables 1-10. The sequences in each Table include genomic sequence, mRNA and coding sequences for both mouse and human. N/A indicates a gene that has been identified, but for which there has not been a name ascribed. The different sequences are assigned the following SEQ ID Nos:

Table 1 (mouse gene: Rorc; human gene RORC)

Mouse genomic sequence (SEQ ID NO: 1)

Mouse mRNA sequence (SEQ ID NO: 2)

Mouse coding sequence (SEQ ID NO: 3)

Human genomic sequence (SEQ ID NO: 4)

Human mRNA sequence (SEQ ID NO: 5)

Human coding sequence (SEQ ID NO: 6)

Table 2 (mouse gene mCG15938; human gene BAT1)

Mouse genomic sequence (SEQ ID NO: 7)

Mouse mRNA sequence (SEQ ID NO: 8)

Mouse coding sequence (SEQ ID NO: 9)

Human genomic sequence (SEQ ID NO: 10)

Human mRNA sequence (SEQ ID NO: 11)

Human coding sequence (SEQ ID NO: 12)

Table 3 (mouse gene: lqgapl; human gene IQGAP1)

Mouse genomic sequence (SEQ ID NO: 13)

Mouse mRNA sequence (SEQ ID NO: 14)

Mouse coding sequence (SEQ ID NO: 15)

Human genomic sequence (SEQ ID NO: 16)

Human mRNA sequence (SEQ ID NO: 17)

Human coding sequence (SEQ ID NO: 18)

Table 4 (mouse gene Zpf29; human gene: hCG27579)

Mouse genomic sequence (SEQ ID NO: 19)

Mouse mRNA sequence (SEQ ID NO: 20)

Mouse coding sequence (SEQ ID NO: 21)

Human genomic sequence (SEQ ID NO: 22)

Human mRNA sequence (SEQ ID NO: 23)

Human coding sequence (SEQ ID NO: 24)

Table 5 (mouse gene: Kcnj9; human gene: KCNJ9)

Mouse genomic sequence (SEQ ID NO: 25)

Mouse mRNA sequence (SEQ ID NO: 26)

Mouse coding sequence (SEQ ID NO: 27)

Human genomic sequence (SEQ ID NO: 28)

Human mRNA sequence (SEQ ID NO: 29)

Human coding sequence (SEQ ID NO: 30)

Table 6 (mouse gene: Ppp3cc; human gene: PPP3CC)

Mouse genomic sequence (SEQ ID NO: 31)

Mouse mRNA sequence (SEQ ID NO: 32)

Mouse coding sequence (SEQ ID NO: 33)

Human genomic sequence (SEQ ID NO: 34)

Human mRNA sequence (SEQ ID NO: 35)

Human coding sequence (SEQ ID NO: 36)

Table 7 (mouse gene: mCG9110; human gene: hCG27579)

Mouse genomic sequence (SEQ ID NO: 37)

Mouse mRNA sequence (SEQ ID NO: 38)

Mouse coding sequence (SEQ ID NO: 39)

Human genomic sequence (SEQ ID NO: 40)

Human mRNA sequence (SEQ ID NO: 41)

Human coding sequence (SEQ ID NO: 42)

Table 8 (mouse gene: mCG2257; human gene: PRDM11)

Mouse genomic sequence (SEQ ID NO: 43)

Mouse mRNA sequence (SEQ ID NO: 44)

Mouse coding sequence (SEQ ID NO: 45)

Human genomic sequence (SEQ ID NO: 46)

Human mRNA sequence (SEQ ID NO: 47)

Human coding sequence (SEQ ID NO: 48)

Table 9 (mouse gene: mCG17918; human gene: hCG23764)

Mouse genomic sequence (SEQ ID NO: 49)

Mouse mRNA sequence (SEQ ID NO: 50)

Mouse coding sequence (SEQ ID NO: 51)

Human genomic sequence (SEQ ID NO: 52)

Human mRNA sequence (SEQ ID NO: 53)

Human coding sequence (SEQ ID NO: 54)

Table 10 (mouse gene: Lfng; human gene: LFNG)

Mouse genomic sequence (SEQ ID NO: 55)

Mouse mRNA sequence (SEQ ID NO: 56)

Mouse coding sequence (SEQ ID NO: 57)

Human genomic sequence (SEQ ID NO: 58)

Human mRNA sequence (SEQ ID NO: 59)

Human coding sequence (SEQ ID NO: 60)

TABLE 1

MOUSE NOMENCLATURE
ICSGNM Rorc
Celera mCG5011
HUMAN NOMENCLATURE
HGNC RORC
Celera hCG16918
MOUSE SEQUENCE - GENOMIC
TCTAACACTGAAGTGGGTGGAACATCCTTAGCAATAGGAAGTCTAAATACTTAGCCATACAAGGCCTCCTTCTGAAAATCATTTTAAGATTATT
TCTAAGACGTATTTTATGTGAATGTTTTGCCTGTGTGTATGTATGTATGTATGTGCACCACGTGCATGCCTGGTGCCTGCAGAGGTCAGAAGAG
GGTGTTGGATCCCTTGGAACTGGAGTTGTGGATGAGTGTGAATTACCATAGGGGTGCTGGGAGCCACAGCTTCTGCTGGACCAACAAGTGCTCT
TAACCATTGAGCCATCTCCAGACTCTGAAAAAACCTTCTGTCTGGTCTCGTAATCCATTTCTCCAGTTTCCAGACTTCACCTGTTCTTTTACCT
GCTTATATATGCCAGCCTCGTGCCCCATGAGTGTGGGGACAAGGCCACAGAGGCAGACAGCTAAGTGTTTGCTCTCCGCAGCAGCATCGATTCG
CATCCTCTTCTGTTCTCTCAGCGCACCTCAGGCCAGAGCCTCCAGCCGGGAGGCTGAACTTGGACCTTCTCCGCCTCGGTGTTCCTTTACCCCC
ACCCACTGCATGTGGCTCTTGGAGAAAGCCGGTTATAGGGTACGGACCGCAGAGGCCCGGGCGCTGCAGGCGCACCCCAGCCTGGTCCCCAAGC
GCCAGGCCCGGGGCTCGCCCTCGCGCTGCAACCCTAATGTCCTCACCCCCGACCGCATCCCACAGTTCTTCATACCGCCTCGGCTCCGGGACCC
AAGAGGCGCCGAGGGCAGGGTGGACCGCAACCCGGGCGGCCGGAACCTCCCGGTGGCCTGCTCGCTGCCGCACCTGGCGGGCCGCGAGGGCTGG
GCCTTCCTGCCCGAGAGCCCGCACACGCGTCGCCGCGAGTCCTTGTTCCACGGGCCGCGCGGCCTGGCTGCAGGCCTGGCCCCGGCGCAGTCAC
GGCTGCACGTCTCGGCCCCCGACCTCCGCCTCTGCCGGGCCCCAGACAGCGACACGGCCTCGTCGCCGGACTCCTCGCCCTGCGGCTCCCCGCA
CACGCCCAGGCCGCAGTCCCTGTCCCCCGACGAGGCCAGCTCGGCGGACACTAGTCCGTACGCGCCGCGCCGTGCGCCACCGCTCTTCCACCTG
GACTTCCTCTGCTGCCAACTGCGGCCGACCAAGGACAGCGTGCTGCGCCTGGGGCCCCGCGGCGGGCAGCTGCGCCTGTCCACCGAGTACCAGG
CGGGGCCCGGGCGGCTGAGGCTGCGCCTGGTGAGCGCCGAGGGGCTGCCTCGGCCGCGGACCCGCCCCCGGGAGCGGTGGCGGCGGCTGCTGCG
TGATTCTGCGGCTGCAGCCGCGCTGTTAGGCCTGGAGCTCAGCTGGAGCCGGGTGGTCCAGGGCAGCTGCAACCCTATCTTCAACGAAGACTTC
TTCTTCGAGGGGCTGCGCCCGCCGGATCTGGCCGTCCGCAGTCTGAGGGCCAAAGTGCTGGACAGGGGCGCGGGGCTGCGCAGGGACGTGCTGC
TGGGGGAATGTGAGACGCCCCTCATCGCCCTGCTGCCCCCACTGGCTGGAGGTCTAGGCCCTGGGTCCTCCCTGGCACCTACTCATCTCAGCCT
GTAGACTGATAGACACCACAGCTTTCTTGGGAGGTTTCCACTGGGTCTGCAGACTTCATCCTTGCCACCTGCCCGGCATGTATTTATTTTTGTT
AATAAAACATCAGTTTGTCTCTAGCTGCATGCTTTCCAGTGGGCACCAAAAACTCTAGGCTTTGCAGCAAGTCTTTTCCACCCAGCCCTTCCTT
AAGCAGTGCTTGAGACCCGGAATCCCTGGAAGTGCTTGTTAAACATGGAGAGCCTAGAGTCCATCCCCAAGCGAGTCTGCTTCAAGAGTCCTAG
ATTGAGTTGGCTCTAGGTGCCTGTCTTAGCTTTATTTCCGTTGTTGTGATAAACTATCTCCCCATCACACACACACACACACACACACACACAC
ACACACACACACACACAAAAGCAGCTTTTATGGAGAAAGGGTTCATTTGGCTTACAATTCTAGATGATAGTACACCATTGTGGAGAAGTTATTG
CCAGGACTTGAAGCAGCTAGTCACTTCCACAGTGAGGAGCAGGGAGAGAGACAATACAAGCTGGAGAGCTGGTTCAGTGGTTAAATGCAAAGGA
CTCAAGTTCAGTTCCCAGCACCCACATCAGGCAGCTCATAACTGCCTATCAAACTACAGTTACAGGGGATCTAATGCCCTCTTGTGGCTTATAA
AGGTCAGGTGGGTGGGTACGTGCATATGAGCGTGCGTGTGTGTGTGTGTGTGTGTGTGTGTGTGTGTGTGTAATGCCCAGGGGGAGTGGCACAT
GCCTTTAATCTCAGCACTCTAGATGCAGAGGCAAGCAGAGGCAGGTGGATCTCTGTGAGTTCAAGGACAGTCTGGTCTAAAGAGTGAGTTAGGA
TAACCAAGGCAACAGAAAGAAACTTTATCTGGGGCTGGAGAGATGGTTCAGAGGTTAAGAGCACTGGCTGCTGCTCTTCCTGAGTTCAATTCCC
AGCAACCACATGGTGGCTCATAACCATCTATAATGAGATCTGATCCCCTTCTGGCATGCAGGCACACATGTAAGCAGAATGCTGTATACACAAT
AAGTAAATGTGTTTTTTAAAAAGAAGAGAAAAAAAAACGTTGTCTGGAAAAAAGAACAAAATAATAACAGAGCTTGGTGGCTCATGCCTCTAAT
CTTAGCACCTGGAGGGCAGGGGCAGGTAGTTCTCTGAAAATTGGAAGCTAGTTTGATCTACAAAGCAAGTTCCCGGCCAGCAAGGCCTGTGTAG
TGAAATTCTATCTCAAAAAACAAAACAAAAAATTAAAGATCAGAGAGAGAAATGAACATAATGTGTGCATGCATGCTTCTAATGCTTACTTAGT
TCAGTTTCTCCACAGTCCAGGACCCAAAGCCAGGGGAATGGAACCACCTACAGTGGGTCAGTCTTCCACATCAAGTAATTGCAATCAAGAGAAT
CCCCCACTGACATACCCACAGGCCAACATGATCTAGACTTTTCTCTTCCCAAGAGATTCTAGACTGTGTCAAGTTGACAATTAAAACTAACCAT
CACAGGTCCCTCGAGTCTAACAAAACCCTGCGCTTTCAGAGAGCCCTTGAGTCCTGGTGTCACCCCAGCAAGAGCAGAAAGCCACCCTCTCCTA
AAGGTCCCTGTGTCTGAAGAGAAGAAACTGGCCTTTCCTACCCCTTAGATCAATATTTGTCCTAAAGACAAATGAAATTTTTGAACCTTAAGAG
TGAGCCAGGCGAGCACAGACTAAACACACAGTGCCCTTTAACCCTTCCTGCCTCCCTCTGGCTAGTCCATACACCTGTGCACCTGTGCAGGAGC
AGGAAGAATGGCTGGAGTCAAGAACCAAAGAGTAAACCAGGTATGCTAGCAAACACATTTAATCCCAGCACTCAGCGGCAGAGACAAGAAGATT
TCTGAGTTCAAGTCTACAGAGCAAGTTCCAAGACATACAGACACCCTACAGGGGCTGGGGGGCCAGGGGAGAAGCACAGAATAGTGCTTGGGTA
CCCATAAGTACTTGGGATTATAGGGACTGGGATCTGGGACAGAGGATACTATCAAGGGAGTGAGTGCTGTTATCAGGGATGCCAGAAAAGGCTA
CTCCAGCCAGAAGATGAGGGTGAAAGACAGTGGAATAAAAGTGGGTCATCTTGTGTTTACCTCCTACCTGAAAGCCCTTGACGTCAGCAGTGAC
CTTCTCTCTGCCTCTTCCTTCCTGGGAACTTGTTCCTCCTGCCAGACTTCGAAGAGGGATAGAGAAAGGCAGGTACACAGCAGGCCCTAGACCA
CTTCCTCCTTCCTCTTAGCCTTTCTCACTTCCCATGGTGCCAATTGTCCCCGTATAGGACCTGCTTCTTCTTAACAAAAATCTCAGCAGGGGCA
GCTACACATACACACACACACACACACACACACACACACACACACACACACAAGTCACCCTTCTCAAGCTCCTTCTACCTCCACTAGGTTCCCA
CCACCAATCCCCAGGCCAAAGAAACCTTGCTCCAGTTGTCCACCAGATGGCAGCATTGACCAAACAATCCTCCGTGCTGACAGCAACCTGATCA
ATGGGTAGGATGGACAGCTTCAAACATTAGTTCCTCCTGGCAACTGCCTCTAAGGTGAGTGTACAGTTGTTATTCAACATGACCTTCGCCCTTA
GACAGATAATAAAGAAGAAAACTAGGTGACATGACTCAAGGTACTTGTCACGAAGTCTGACAGTCTTGAGTTTAATCCTAGAGATCCACTTGGT
GGAAGGAGACCAATACACACATATAATGTAACTTCTAAAATCTGTATTAAAAGCCTGAAGGGTTTCTTTTTCCCGGGATAATAGCTCTTCAGTC
ACTTCACAACCTGGCACTTCGCACTTAAACCTGTGAACTCTGAGAAGTCCGCCCACTCCCCAAGCTCTATCTGTGCTGTGCTGTGCTGTGCTCT
GTGTGTACATCGGCCCTCAAGTTCTTTCATAACAATCCCTAGTGAGCAATGCAACTTGGAAGTATCTGGAAGGATGTCTATAGGTCAGTTTGCC
CGCCTTCATCAGTTCCTCCAGCAATGGTGGGTGCTATAGGATGCCAGGCACTGGGGCCAGCAGGCTTGAAAACAAACGCAGGAATCAAGTGAGT
CAGAAAATGAACAAATATCAGTAAAGAACTAGCTGGCGATGGGCCGTAGAGAAAAGTAAAGTAAGAAAGGGGAGGGAGGCAAAATTACAGGAAT
GGAAAAGAGAGGGAGGGCCTGAAATACTTTTTTAGCTGCCATGTGTACTTTGTACCAGGCCTTGCACTTTACCCGCTGTCTTATCTCACCACCA
CTGGGTAAAGTAGGCATTACCAGTTCACAAAGGCAGAAAGAGATCCGAGAGGTTAAATAAACTTGCCTGAGGATTGGCTTTGGCTGTGAGGAAG
GCTTCTAACAGCGACTCTATACCAACATCCTCTGCCACAACCCAGCCCAGGAATTTGGGGAGTCTATGAGATAGAAAACACCTCCCCCCCAAAA
AAATGTTCTGTCACATTCCCAGCCCCCGGGGCTCAGACAGGTACACCGAGTCCCCCAGTCTCAGATACATACCATGGGAGCAAATGAAGCCTAA
GAACCCATCCTGTGTGCAAGGAGCTGAGGCCTCTAAGTACCGCCATTAGCACAGTACTGCCACCAACGCAGGTCAGCACCATGGTTCTCCCCCT
TTTTAGCTGTGACAGGATTCAGGGCGCATGGGTGACACCCAGCAAACCACACTGTTAAAGTCTTCCTTGTCCCCCAAACCAGACCTCCCAATTT
TGTTCCTCGTAATTGTTTTTGAGGCATACTCTAATGTAGCCCAGGCTAGCCTCAAACTCCCTACCCAGCTGAGGATGGCAGTGAACTCCTCCTG
CTCTTCCAGCCTCTACCTTCCAAGGCTAGACATACAGTAAAGCCCTGCCACCGTGTCACGTTATGAGGTGCTGTAGGTTGAATCTCGGGCTCCA
TCTCCAGCCCTCCACTCCTGATTTTTCAATCCTCCCACCTCCAGTCCCACCTCAGTGTTAATTGGGAAATGAGAGCTATAAGCAGTTCCAGGGT
AGGGCTAAAGTCCATCTTTCCCGCTACTCCTCCATACTGGGGCTCTACATAGGTACCAACGGGGATGCAGAAACCACGGATGTGCCAAAGAGCT
TGCAATGTCTTTTTTTTTTTTAATGGCCAAGATTTCATCCACTCCCCAAGTCATTCTGGGGAGTTGAAGTCTCCGTAAGCCTGAAGCTCAAAGA
AAGGTAGTCTCTACTGCACTGCAGTAGAGAGGCTCCAGTGAAAGATGGCGCCCTCTGGGCCAAGCCAGCCTCTGCATGCTGCCCAAACCCCAGA
GGTTTCCTTTTTACCTGCTGCCTTGGGTCCTCCCTGGAAGGACAGGACTGTGCTGAGCTTTAAGTGGTTTCTGCCGTTTTAAGGGGTGGGGAGG
AAGGGAAAGAAGAGTTGGTGACTGCTCCCCATTTCCAAGCCGCCGCCGCCACCACCCACCTCCAGGTTGTTTGCCCCCTGCTTTGAGATTCCTG
GGCCTGATAAGAGGACTGGGCACGTGGGGTAGAGTGATTCTCTGATCCCTATCAGCCTCTTCCTTGCATAAGAATGTATTTGAGTTTGCTAGGC
CCGGCATAGTCTCACAAGAGAGAGCTATATCTGGGTCCTTTCAGCAAAATCTTGCTAGTGTATGCAATGGTGTCAGCATTTGGAAGCTGATTAT
GGGATGGATCCCTGCATATGGCAATCACTAGATGGTCCATCCTTTCGTCACAGCTCCAAATTTTGTCTCTGTAACTCCTTCTATGGGTGTTTTG
TAGAAGTGGATGATCACAGTCAGCTATTGGATGGGTCACAGGGCCCCCAATGGAGGAGCTAGAGAAATTACCCAAGGAGCTAAAGGGAACTGCA
ACCCTATAGGTGGAATAACAATATGAACTAACCAGTACCCGGGAGCTCTTGTCTTTAGCTGCATATGTATCAAAAGATGGCCTAGTTGGCCATC
ACTGCAAAGAGAGGCCCATTGGACTTGCAAACTTTATGTGCCCCAGTACAGGGGAACGCCAGGGCCAAAAAGGGGGAGTGGGTGGGTAGGGGAT
TGGGGGGGGTGGGTATGGGGGACCTTTGGGATAGCATTGAAAATGTAAACGAGGAAAATACCTAATAAAAAAAAAAAGAAAAAAAAGAATGTAT
TTGAGGAGGAAATAGCCCCTGCTCTGCTTTAGAAAGCACTGTTCCTACAGAAAAGAGCCTTGGTTACAGCAAGCCAGGGGTGGGGAGCTGGGGG
TGGGGGTGGGGGTCAGGCAAGAACACCCAGGGGGCATAATCACCACAACCTACTCTGAGCTTTACCAGCCCGGTAAATCCCAAGGTCACACTGG
ATGACAGCTGTACACTCACCCTATGGCCACTGTGGGAGGAACAAATAGTTGAAGCTGTTGGAACAGTTCCTCCTGTGGGCGTGATCTCCTGATA
GCTGGAAGCACAGAGAACTCTCTGGTGGCAGTATCAGTCTGAATAGGGAACCCCCACCCTAATCACCCACCCCCCCATGCTGGAGGCAGGGCCA
AAAGCTGGGTAGGATGATGCCTGTAATCCCAGCCCTCAGAAGGCTGAAGGAACTGGATAGCCATGAATGTTCAAGGCCATCTTGGACTACTGAG
TGAGATCATCTTAGCAACACGTAGGAATGAAGCTCTCGAATGCTGAAATGAAAGAGTAGCTTAGAAGAAAGCCACTCTCAAGCCAGGACCCGGT
CGCTTCCTGAGCCCTCACAGTCATTCTGACAGTGCCAGGCAGTGTCTGCCACTGCCATACTGTGTGGCATCTGAAAGCATCCCTAGGGGCCTCC
ACCCTGTGCCTCTAGCTTGAGGGAGGGGTCATCTGGAAGGTCTTAAGACCCATAATCACCAAGAGAAAAGGACCTGGTCAGCGATGCAGGTCTG
TAACCCCAGCACACCCAAGGCAATGTTGTGAGTTGGAGGTCACCCTAGGAAAATGAGAGGAGGGGCGGGGTTCAGCAGTTAAGAGCACAGGCTG
CTCTTCCAGAAGAACCCAGGTTCAATTCCCAGCATCCACACAGTGGCTCACAGTCATGTATGACTCCAGTTCCAGGGCACCTGATATCTTTTTC
TTTCCTGCATAGTGAACCAAACACACAAATGGTGCACAAATAAACATGCAGGCAAAACACCCATACTATTAAAAAAAGATAAGTGAAAAGAAGA
AAGAAGAACAATAACTAAGGGAGATGAAAGCGAGCTGGTAAGGCACAGAAAACTGAGCAGTGATGGGCAGATAGGAGCACAGAAGAGCAGGAGG
GGATAGCTCTGGGATAGAGGCAGGCCATTAAGGTCCCCATCCTTTGAGTGCCCTAACCTTTCCAGAAGCCTCTTTCTGACTCTGTTCCTAGGTA
GGGGTGCCATCACTTTGCCTCTTTTCAAAGCTCTCCAGATTGGCATTGTGTTTTGAGAAGAACAGTATACATGGGCCTGTTTACCACCAAAGAC
CGTGAAGTCAGAGATGACCTAGTCAGTGGAGTACTGCCACAAACACACTGGGGTCAAGGGTTTTGGGGGGTTTTTTGGTTTTTTGGTTTTTGTT
TTTTAAGATTTATTTATTTTATGTATATGAGCACACTATCACTCTCTTCAGACACACCAGAAGAGGGCATCAGATCCCATTACAGATGGTTGTG
AGCCACCATGTGGTTGCTGGGAATTGAACTCAGGACCTCTGGAAGAGCAGTCAGTGCTCTTAACCACTGAGCCATCTCTCCTACCCAAGGGTCA
AGGTTTAATCCCCCAGAACCCACATCACAAAATGGGTACAGTGGTAGATAGGTAGGTAGGTACTTGTAGTCTCAACAATGGGGTCGTAGAAACC
CTAGAAATCACTTGCCAATCTGGCTAACCAAGATGGCGAGTTCTCCGTTCCATGGGAGACCCTGTCTCAAAAAAACAAAACAAAACAAAACAAA
AAGGTAAAAGGTAATTGAGGCAGACATCAAATTTTAATCTCTATCCTCCACAGACATGCACACTCACACGTGTACACACACCCACCTATACAGA
CACACACATATATGTATACATATAAAGGACTTAGGACTAGTCAAAGCCTCAAAGGCCCTCAAAAGTCACCCTACACCCCTACACTTTTCAGTCT
CCCTGGGGCTAAGAACGGCTATTCCTCCTAATCTTTGTCTCTGACTCTTAGGAGACTCAGCTCCCTAGAGCACTGGGTGCTACTGGTTGCACAG
TGTGAAGCTCCACGGAGGGCCAACGGTGGAGAATGGAATGCTGGGAGCGAAGGGACAGCTGCCTGCCCTCTCCACGCTAGGTGCCACGTGCACC
AGGTGGAGGGAGTGGGCGAGTCACGAGGCCCTGGCGTGGCCGCCTCCTGCCCTGCTGTTTACCAGCTGGAGAAAGCAGGAGGAGGGGTTGGGAG
CCTCCTGCTCCCAGCCTGGCCCCTAGCCAGCGGTTGTTGGGTAAGCAGGAAAGCCAGGCACAGCTAATCAAACTCTGGGCAAAGGACATGCCGA
ACTGAGGCATCCAATATTCACAGAAGTCTTGGCTCTTGGCCCTTCTCAGACTCCAGTGCAGGCCTTTATGTGAACAGAATTACGGGGTCGTGGG
GGAGGGGGAAGGGAGAACCGGGAGGAGGGGGGAGGATTCCCTCCTCTCAACTTGTTGGCCCAGTGCAGTTGCTCTCTTTTGCTCTCCCTCCAAC
ACCCCTCACCTCTCAATTTGCTCTTCCTCCTCCACACCTCCCCCCACCACACCAGAAGTATGGGAAGCATCTAGAAGCCTGGAAAATCCAGAGG
TGGGCAGCATGGAGTTAGAGAGAGGCAAAGCAAGACATGTGCTGGGGGAACCCAAGCTGCAGGGAAGTATATCTGATCCCATCCTGGAGATCAG
AGTGCACACACACACACACACACACACACACACACAGGCTGTCTTTTCTCTTCACTCTTCCTGCCTCCCTTTCTTGCTATTCTGTCACCTGTCC
TAAAAAAGTTACAGAGACCAGGTTCAAATCTCAACTCAATCACGAATTGCCCTTGTGAATTCCTGGCAAGTCTTTTCTATTCTGGGCAGCAATT
GGTTCATGATTATGAATAATCACTCGTGCCTGTAGGGGGTTATCATGTGGGATTCCAAAAAACAAACAAAAAAATTCTGATTTACCTACAAGGC
CTGGCAAAAACTCAGCATACAGTAGGTAATTAAAAAATACTAGCTCCCTTTCACCTGTTTTTCCTCAAGACTCCAGACACTGGTCCACTGCTGT
CTCCCTGAGTCCCCTCCTGTGCCAGGACACTCTGCCAGCCACTCCTTTCCCCTGCCTGCTGACGGGCCAGGTGCTCCCTCCCTCTTCCCTCCTC
CCTCCCTTGGGCCCTGCTCCCTCCCCTCCTGGGCAGCCAGGGCAGCAAGGACGGCACCAAGGGAGCTACCCCATGGACAGGGCCCCACAGAGAC
ACCACCGGACATCTCGGGGTAAGAGACCCTAAAGCCCTGCAGGCAGGTGGAAGGGACTGTCAGGGGGCTCAGAGGAGGGGAGGGAGGCGAAGAG
ACTGGGAAAGAAAGAACTGGGGAGAAGAGAGGTGAGCAGAGGGGAGATGGACTGGCAGACAGAGCAAAACGGTGAGATAGGACCGAGTCTTTAC
ATAACTGTAGGGGGGGGACAGGAAGAGGGCCTAGGGTAACAGCAGGAGACCTGGGAAGACGGGGAGGGCAAGAAGGAAGAGACAGGTTGGAGTG
GGGGCAGGAGGGAGGAGCTGTGTTAGGGAGGCTTGATCGAGAGGACGGGAATGGGGCTGGCTCCCAGGTGTGAGCAGGCCCCTCCCTGCCTGCC
ACAGAATCCTGGGCAGCAGCGGCTGGAAGGGTGACTCTCCGCCTTCCTATTCATTCACAAGTGGCTGCCGTTCTGGCGCAGGGGCTGGGGGTGG
GGGACCTGGGAGAGAAGATGCAGTTGCACCATGCCAGCTGCTCTAGCCCAGCAGTCCCACGCTTTCCTTCAAGCTGGTCCGAGCACGGAGTTTT
ATCTTTGCTGTGTGAGGCACCTGTGGAGCACAGAAAGGAACTGGGGAACAGTCCCCCGATGGAGTGAGGAGGGAGCCCTTCAGAACAGGACATC
CCCTCCCTCCCTCCCATGCTTGTCAGCTGGGAACCTAGAGAGAGTGAACGACAGAAGTGCCAGCTTCTAGGGCCACTAGAGAAAGGCTCCGGGG
TGGGAGTGGGAGTGGGGGCCAACAAGGGGAGTGTCCCCATGCAAGACTGCTCTGAATTGCAAAGTTGGAGGCTGGACAAGGGTGCCACGTCAGG
GAGGAAACTGAGTAGGGCCAGACAGGATTAGCAGGGAGGTGGGCATGGCATGAAGCCACACCCTCTCTGGGCAGACCTTCCCCTCCCCCACCCG
GGGAACAGGCTACCAGGCCCCATTTACTATCACGGCAATGAAATGCAGGAAGCCTGGCTGCAGAGTGCATCTCCTGGCAGAGGGACAGTGAAGG
AGGGACAGGACATGTGTGCCAGGCTTGGACAGCAGAAGGCAAAAAGTAACAGAAAGGAATCAAAGGTTAGGGACATTACAGACAGGGCTAATGG
GCTTTCTGGAACAGAAGACTGGCCCTAGGCACCCTGGATATAGGAAATGGGTTGCGGCCTCTTTTGCCAAGGGCTCCCTCTCTAGGTGTTTCCC
AAATACTGACTGCATGCTGATTTCATCCCAGATATGTAAACAGCCCTGTAACTATGCAGTAACCATGACTCTCCCCTCAGCTGCTGCTCCCCTG
GACAGCAGAGCATCTCTGTAAACCTCCACGAACCATGGGGTGCCTCCTCCTGTCTGATCTCCACCCAGACCCATCCACGAGCAAAGCAGCAAGG
CAAAGGGACCTTCCCTCTGCAGGCTTGTGGGCAGGTGAAGCACAGTGGCGGCCAGAGAGGGTCTATCTGCAGCTCGGCCCTCCTTGTCTTCCTC
TTCGCCCACCCTCCCCATGCACTCCCAGCCCCACACCTCTGCAGCTCCCTCTTGCTTTCAGGCACAGATAAGCAACCAAAAGACTGTATTCCCT
CTGGGTGCAGAATATGTGTGTACGTCAAATGCACCAGATACTAGAGGGTTCTTGCTGTTCCCCAGAGCAACAAGCTGAACAAGACAGACAGAGG
CTCTGTCTCCAGACGGGCTTACAATATAGAAGGGATGATGGCATACCTAGGGCAGTTGTCTGTTACGTCAAGTGCCAGGAAACAATGCAGAGTG
CTGGCTGAGTAAACCCCAGGGCAGGGGACAGGGCATGGGAGGCTGCCCCAGAGGAAAAGAGAGACATCTGAGATCTAGCTAAATGGGGGAGGGA
GGGTAGAGTGTGACATGAGGAAGTCCTGGAGGCAGAGAGGAGCTGGGCACTTTCAGGACCTGGAAGAGGGCCAGTGTGGCCAAAGGGCAAAGTG
ATGGGCTCTGCCAAGGGGCCCAGCTTCTCAGAGGCCAGGTAGTGCTGGGGTAAAAATGGGGCAGGGCAGGAGCCAGGAACTGGGGAAGTCTTTG
GCTTTTCCCCTAACATCTTCAACTATTCACTGATCCCTTCCTTCTCTTTACAGAGCTGCTGGCTGCAAAGAAGACCCACACCTGTGAGTAGGGG
TTTGGGCCAAGAAGGTGGCATGGGGTATGGCAAAAGCATTCAGTTCAGGAGGCATGAGTGAAAGAACACCCGGGACTGGACAGAATTGGAAGAA
TCAGGCTAAGACTGCAAAGCCCAAGAGATAAGGGCCAAAGGGAAAGACTTTAGGCCAGGACCAAAAAAAAAAAAAGAAAAAAAAAAGAAGCCCA
AGGGGTGCCAAGTTACAAGCATAGAAAGAAAAGGGGAACTGGGAAAGAGGAGGGCAGGGTGCTGGACAGAGGCTCAGCTGCTGTGTCTGACCAG
GGAGGAGGTGAAGGCCACCCCAGCCTCAGAACTCAGCAGCCACAATAGGAGGCCTGGGAGCGTGCCCAGGGCTTTGTGGTGGCAGAGCCAGGTT
TGGTGTTCATCTCTGTGGTTAACACATTCTGAGGTGTGAGCAAGCCTGGAGAGAGGCTCTGCAGAGCTAGAGAGCAGGAAAGCCTTGTCTCTGT
AAGAATGAGTGGACAGGAACCCGAAATCCTCAGACTGCAGTGTACGAGGAAGAGACCAGAGTTCCAGAAGGGCTTCCCAATGGACACTTGCAAG
TAAACATCTGTCTCAGGCCAGTGCAGCACCCCCAATCCAGCCACATAGAGAGAAGCTATTCCCCTACAAAATGTGCCCGCTCCACCAAAGTCCT
CCCCAGGGGTTCGTGCATACCCTCAGCACCTTGGGGGCACTCTGGAAAAGCCACAAGCATGGTCATTAAGTTAATAGTGACAATCTCATCAGAG
GAGGTCACCTCTACTCTTCCATCACATACTCATTGGTTGCCCACCAATCTGTCCCCACTACAAAAGCCATCTGTGGCCTTCCTGTCCCAGCATC
CCTCCTCCCTGAGCTTGGGGCTCCATCCTCCTGGGGGGGTCAGAGCTGCTTGGCTCAGCATAATCCTGATCAGCCTCCTGTGCACTAGCCACAG
GGCCCCACGGAAACGCTGAGCAGGGCCCTGGGAGGAAAGCGAGTCAGAATGACGCAGGCAAGCTGGGGGAGGGGACCAGGGACCACACTTGGGG
GTGTAGAGGGGGACTTTTGGAGAGTGGAAACTGGGAGAGACCAAGCCAAGCTTTTAGACCTGCCTCCCATCCTTTAACCCTTACCATGTGTCCT
CCTGAGGATCCAGGCTAGTTACCCTCATCCATCCAATGTCCAACTCCCAAGCTCGCAGAGAGAGCTTTTTACTAACCAGAAAGATTGAAGACCT
CTCAAGGAGGTTGAGCTCCTTGAGTTTTTATTGTCTTGGCCCCAACCCCAGGCCAAGACAATAAAAACTGCCTCCCAAGTGAAATAACCTCAAG
AAAGGGGTCCAAGCTGAGAAACCCGGGGGCACACCCAGACCAAGAGGGGACCGAATGACTAAGGGGCCAGAAGAGGAACCTCCAAGGCTGAGGT
ACCCCACAGAGCAGATGGAAAACAAATGAATCCATCAATAAGAAATCTCACACCAACAAAACCTGAAGAGAAACTCAAGTGCCAGGAGACGGGC
CAGTCTGAGACACACTCGAATGCAAACCTGAACCAGAGATACCCTTCTATCCCTCCAGTGGATCTGAGGTCACTCTACCTGGCCATAAGTCCTT
ACTCAACCATTCACTACTGTGTGATCTTGAGCAGGTTACTTAATCTCTCTGTGTCTCACCTTCCTTGTCTTTGGAATGGTAGTTATCACATAAA
GTATTTTATGGGGATTATGAAGTCCACACAGAGCTGAATCATTCCCAACACTGAATTAAGCACTGCTGCTAAGAGGATGAAGATGGTAGTGTCA
CTATCTGTGTCCCAGACTAGCAGTCTTGTTTCCAAAGGCCTTTCTGAAGGACCAACCCATCCTACAGATCCTACAGAGGGTATTTGCCTGATGA
TACTGCCGCTATCGTTTCCTCAGCCCAGGATAGCATCATGTGTAGGAGGAATTTGGGTGTGGTGAGGGCAGCCTCCCGGTTGTACCACACTGGT
TATGGCTGTTGACAGTCCACAGGGTCTCAGTGCCACAAGACACCACCCAAGACAGATTCCTGTTACTTACCTTTGGAACATCCCTACCCCTGAT
GTCACTTGGTGAAGAGGGCCTGGGGTACTCAGGAGAGGGGATTTCGAGCCTGGCCTCTGCCCATGAGCTCCACGAAGCTCCCCAGCTAGAACTC
CCCTGGCGAGTGGAAACAGCTTTTACCGCGGCTGTAGCTGTGGTTTTGGAATTTTCCAACGCCCCCTGTGATTGGCTGCCCCGCCCCTCACACC
CTGCCCCAGACCCAGATTGGCCACGTGGGGTGCCTGTCATCATACCCAATGCACCTCTGGGGGTTGGGGGGCTGTCACTTGGCCACCTGTGTGG
AGCAGAGCTTAAACCCCCCTGCCCAGAAACACTGGGGGAGAGCTTTGTGCAGATCTAAGGGCTGAGGCACCCGCTGAGAGGGCTTCACCCCACC
TCCACTGCCAGCTGTGTGCTGTCCTGGGCTACCCTACTGAGGAGGACAGGGAGCCAAGTTCTCAGTCATGAGAAGTAAGTGAATGGGGGCATCC
GGTCATGGGGGAGCCTGGGTCCTGTCACCATTCCTAGGCCCGCTGAATAGGAGTGTATCTTGCAAACCGTGCCTCTTTGCCAGGGTGTGTCCCA
GTCAAGGTCAAGATCTGCTGGGAGATGGGTGGAGTCCCAAGAGAGTTACTCTTCCCTGGCAGCTTCCTTGGCAGCAATGGTAATCAAATTATTG
AGGAGTTCCTGACATAAGTTCTGAAGTTCTGGGAAGACTGAGCATGCCCAAGGGTCCAACCCTTTGATTTACAAAGAGACCAAGACCCAAGAGG
GGAGTGCATCACTGAAGAGCTCACCATTAGGGCCTGGGAGCTGGTGGAGTCTGGTAGACATTAGGACCCACAGCTTATATTCCTTGTTCCTAGT
GAAGAGTTTATCTTTGTGCCTCCAACTGCAGGCACTGCTGTACCCTGAGGCAGGAGTCACAGTAAACTGAGGTCAGCAGGCAGTGGGGATCAGA
ACTCATCATCTCCCTGATTCCCTAACCTTAAACACACACACGCACACACTCATACCAGGGCCTGATGGCTCAGTACAATCCAGCTAGGGACTGC
TCTGTGTGTGCTATGTGTATATTTGTGTGTGTGTTTGCATATGCTTCAGGTGCAGTTGATCATCTATAGCAAGAAGAGATGGGAGCAGAAAACC
TTGGTCTCACTCCTTTCCCTGCTTGAAACCATGGGCCCACAGCTGCTGGGCAGATGTCAGGGGCCCTTTAGGCTTCCAGCTGAGCAATGTGGGG
GATCCGATCCTCCCTGCTCATGTAAGGGAAGCCAGAGGACCTCTCAAAGTCTACAGTGAGTGAGGAAAAGGGAAGCCTGTTACCCTGAGGCCTG
AGGAAGCCCCAGCTTTAACCTGGGGTCTTTCCACAAGCCACAAAAGGGGAGGCCATACTGAGTCCTGGAAAGGCCCATGGACTGAAAGGCTAGT
GAGCAGGTTCTAACACAGGGTCACCTGAGGCAAGCCGCTGATCTCTGGTCTCCAGCCACAAAATAAATGCCAAGCATGGGAGTCTCTGAGTTCT
AAGAGCCCAAGACCAAAGAGACCCCCTCGTCCCCTAATGGGTGAAAGAGAATAGACCCCTATAGTATACAAGGTCAAGTTGGGATTCCACTTGC
ATGCATGGGACAGGACTATAGTCCTTGGTGAGACAAGAGGACCAGGAGAAGAACCAAAAGTCCTAAAATGGAGTAGGAGCCGTGGTTCTTTTCT
GGCTTCTTCCCCTCCGCAGCAAAAGCTGGCAGATCGATTTCACAGAAACAGCCCCAGGTCGGCCGAAGCAGAGGGCAACAGCCCCAATAGTATC
TGAGGGATACACTTGGGTAGCTCTGCGTTTTATGCACACATGCACGCGCGCGCGCGCAAACACACACACACACAACACATGCATACACATAAAT
ATATCCAAGTTTTCTGACCCCTTTGAATTCCAGTTACCACCTACTTGGGAGAGGTCAGAGCAGAGAGGGTTTAGGACCATGACACACCAAAGAG
TGAGGCCAGAGAAACAAGGAATAGATGGAATGTAGGAAATAAGCGATGGGTTTGAGGGTCAAGGGGGAAGGGGTCCAGATGGAGCACTACCAGG
AGAGCCATAGAGTAAGAAGTCCAGAGACGAAGGGCACAGGTCATAAAGTACAGACACGGGAGGTGGGAAGGGAACATGGCAGACTGGAGGGTGT
CTCAGGAAGAAACAAAGCCCCTTCGACCCCTACCTCGGCCAGTCAATGGTGTATGACATAGCCATCTCACTGTTAGGAGCTAGTCAGTGTGGAA
TCAGTAGACAGGAAGTCTAGGAATGGGAGGTCACATGCCAGTCCTCTTCCTTGAATACAGCCATGATTATGTTGAGCAACGGGCCACAAAGAGC
TCCGAGTCCCCAGGGTGTGTGTGTGTGTGTGTGTGTGTGTGTGTGTGTGTGTATGTACATTCGGTAGCTGATTCCTTCCTTAGCAATGCTGGAA
ATTGAACTCATGCCACAGGCCTTTGTGGCTAGCACTCTCACCCACCGAGCCTTCTCATGGGCCTTTTATTTGGTTTTGTTTGTGACAAAATGTC
ATAGACTGGCTTTGAACTCCATATACAGCTGAGAGTGACCGTGAATTCCTGATCTGCCTGCCTCTGCCTCCCACGTGCTAGGATTGTAATATAG
AGCATCAGGCCCTGCTCCATCAGCCTTGTTCCTTCCTTGTATCAATCACTCCTGCATCTTTCTCTTTTCCTTTCTTCCCATTCCTACCCAGAAT
CCTGACTGTCCCAGGCTCAAGATTCTAGGATGTGAAGATCTTCAATCTGTTCCATCGCAATGAATATGTGTCTTGATAACTGCTGTCCCTACCA
GGGGCTCACCCACCCCTCCCTCCCCTGCCTCTGTTGCTGACTCAAACCAACAGGGACAGGGACTTTCCCCATGCTGAGTGCTCTCTAAGGAAGA
CAGCCTGGGGTCTCACTCCTGCCCCGTGATTATTTCCTGGGGTCTAATCTCCACTCCTGCTGCAATGATGACACGGCCTCTTCCCTGAGACTCC
AAGGCTCAAAGAGTGGAGTTCTGTTTGGGAGCAGAACAGAAACAGGCAGAATTTCAGTACCACAGCCAGGTCTTCCTGAGTCTGCGTCCCTCCC
AGAGGCAGAGCCAGAGCTCTGGAGGCAGGCAGGCAGGCGAGGCTCCCACTCCTCAGAGCTTCTTCCTCCTGGTGATTCATCCCCTTCCCCCATC
CCCGGCTTGTTTTCTTTGCGTTCTTTTATTTCCTCTCCTTTCTCCAGTCCCAGACTGTGGGGGTGGTGGGGGCAGCAAGAGGGGGTTTCGAGTG
GCTGGCTGGCTGGCTTGCTTTCTTGGCTTTGCAAAACCTGTCTCTCCCTCGCCCACCTGAGTTTTAGAGTCACCAGTTTTTCAGTTCTGATATC
AGGTATGTCAGTAGAAAATTCCATGAGGGCTTGCCTGGTTGGGGACATGCCCAGCAGGTAAATCAGTGGTTCCTGTCCCTGTGGTGGCACCCAC
CACCTGGGTTTCCAGCAGAAACTGGAGGAACTGAGAACTAAGGAATATGGGAGGCTGATGACAGGTTGTGGCACATAGGGGACCACTGGCTGGA
GCTGTGGTCTCAGTGAATAGAGAAGGGGCCTAAGCTCAGAGATGCATTTTCTTTCTCATCCAGCCATTCACTCACTGATGCAAAGAAAGGCCCA
ACAAGGGGCAACCTAGCCCATACCCTCAAGGAGCTGTGGACTTCCAGAGGAGCCCTGGGTCAGGAGCTCTGCTGGGGTGCATGGGTCATGTGAT
CTTGGCTCTAGAGAAGTAGATAAAGGGTGCATGGGCCAGCCAGGACCACTGAGGGAAGCCAGGCCCAAGGAATGGTATCTGTCTGTCGGTTTAT
CTATCTCTCTACCTATCTACCTATCTATGTATCATCTATGTCTCATCTGTCTGTCTGTCTGTCTATATCTTTGTACGAGGCATGAGTCCTGGTT
TATTCCTCACCTAGCCTATCATTACCCTTGTTTCACACTTGATCATTTTGCAGCCAATGACCTTGCTCTATCCGCTCTCTTTTAGTATACTTAG
GTATCTCATCCTCCAAGGGGATACAGCTTAATGATTGAGGACTCTGACTCCAGTTACCAAAACCTGAATGTAAACTCCTTGCTTGCCTTTTCCT
GGCTGTATAGACTCGGTCTAATCACTCTCTAGGCCTCAGTTTTTGTTTTTTTTTAATCTGTAAAATGGGGACAATACTAACAGACCCTACTTTA
CAGAATTGTTCTGATGATTAAAAGTATATGTAAAGAGTCCTCAGGACAATGGAATGGTACTGCAGGACCTTGCCTTGCAATCTTGGCGACTTGA
ATTCAATCTCCAGAACCCATGAGAAGGTAAAAGGAAAAAACAGATTTCACAGAGTTGTCCCCTGACTTCCACATGTAAGCTGTGGCATTTGTGC
CCACATGTATATATAGCATGCACACATGTAGTATAATAATTCATAAATTATTTTTTTAAAGGAAGCTGAGTGTGGTGGTTCCATAATCCCAGCA
CATGGGAGGCTGAAGCAGGAAGATCTTGCATTTCAGGCCAGCCTGAGTACGCAGTGAGACCCTGTTTAAAAGAAGGGCTGGGAGGCAGTGGGGC
AGCTCAGTGGGGAAAGGCATTGCTGTCAAGTTTGATAACCTGAGTGCAATGCCCAGGCCTCACATGGAAGAAGGAGAGCACCAATCCTAGAAGC
TGTCCTCTGACCTCCACACATTCACCGTGGTGTGTGGCTCATGTGCAAGCATAATTAATAAGTGAATTAATTTAGCCAGGCGGTGGTGGCGCAC
ACCTTTAGTCCCAGCACTCGGGAGGCAGAGGCAGGCGGATTTCTAAGTTCCAGGCCAGCCTGGTCTACAAAGTGAGTGCCAGGACAGCCAGGGC
TACACAGAGAAACCCTGTCTCAAAAAAAAAAAAAAAAATTTAGTTGAGGCTGGGGGAATAGCTGTCCTTTCACTCTGCTCCCTTTGTGCCCATG
GGCTGGTGGGCAGTGACCCCTGAGGAAAGGGGAGACCCACACATGAGACCTTGGATTGTGTCCCACAGCACAGCACAAAACACACACACACACA
CTGTAAAGCCTGGCATACACTAAAAATTCAGGCAGTGTCTACATCTTTTGTTATTCTTCCTTCTTACTCAGTATCAGCTGGAAAGCAGGAAAAG
AGGAACAGAAGAGAGCCCTAGCATGCCTCTGCCTAAACCATTCCTTCCCTGAAACTGGGGCTTCTGGAGTGAGACCCTCCTGGGCTGTAGCCCC
TCTCCGTTCCAACCACTTCCTCTCTGTGGCTAGGCACCTGGTCAGGACCCAGGGCCCAGCTCTGGATATAGTTAAAATGCACTATCAATGAATC
TTGCAGCTGTGAGCAGCGTGAGATTAGAGGAGGTTGGGGGCAGAAGTGGCTTGCTTCTACCGTATTTCTCCTCCCTGGAGGCTCTAAGGTGCTC
CTTGAAGAGCGTTTCAGGGCAGAAGCAATCCAAAGGAGAGTCACTTTTCCTTTCCAGGGCTGCAACATAATTATAGTTTTTACATCACATCCGT
AGGGAAGATGCAGAGATAAACTCAGTGAGCTGACCTGTATAAGGAATGGGTTTGGGAAGTGATTATCAAACCTACCTTGACATCTTGGGAGGAG
CAGCCTGGCACAAAAGCAGCCAGAGACTAAAATGTTTTTATGCCAAGCATAGTCTGAAGGCAAAGAATCACTGTCCTGGAGTGCATTATAAGTG
TGGACTTTCCGAGCCCTGCCTTGGGCTGCTTGAACAAAGATCTTCATGGTGGTGGTGGTGTTGTTGTTGTTGTTGTTGTTGTTGTTGTTGTTGT
TGTTTTGTTCCTATATTTTAACAAGACCTCCAGGTTCTCTTTTTTTGAAAGACAAAAGACTAGAGTTGCTAGCTGGGTGCCTCCTGCACCAGGC
ATTTCTGCTTTTGTAAGAATAAAGATAGAGGGGTTTTTTTTTTTTTTTTTCCTTCTTTCAGTTTTTAGAGACAGAGTTTCTCTGTATAGCCCTG
GCTGTCCTGGAACTCACTTTGTAGACCAGGCTGGCCTCGAACTCAGAAATCCTCCTGCCTCTGCCTCCCGAGTGCTGGGATTAAAGGTGTATGC
CACCACTGCCCGGCTGGTTGTCATTTTTAAAACAGGGTCTCAAACTATACGCCAGGCTGGCCTGAAACTCATGGCAACCCACCTGCCCCAGCTT
TCTGAATGTTGAGATTTCCCAGCTTGAGGGTTTTCTTTGTGATACCCTTCAGGAGAAAGGCACAATGAGGGAACAGTAAGACACAAAAATCCTA
AGAAAGAAGCAGTTTGGCTGCTGTGGGAGCCATGGGGCTTGGGCTGACCTGAAGGCTCCTGACCTTTGATTGGCTCCAGTTCCTGGCACCGTTG
GGCCCTGAGATGGTAAGTTCCTGGCAGACTAGGCTCCCAGGCAACAGTTTGAGTAATCCTGTGATTACTCAGCACCCCCTCCCCAAGCGGGGTT
CCCATCACCTGCTGTTCAGGGTCTGAGTCTTGAAGCTTTGCTGTGGAAGATGTTTCTCCGCTCCTCTCATGCTGACTTCCCTACCAGCCCTTCC
CCTCCTTCCTTCCCCAAGTGTCTGTCTCTCTCTGACTTTGTTTGTCTGTCTCTGTTTGTCTCTATCTTCCCACCACACACACACACACACACAC
ACACACACACACACACACACACACACCCCACTGGTACTTTCTTTGATATTTAAGTGTTTAGGGTGGATGGTACTGAACAGGGTGAAGGTGTAGG
GGACGGAGAACTAGAGAAAGTCAAGAAGTTTGGTCATGTCGAGGATTGGGCACAGGACCAGAACAAGCAGGGGTTTCCCACGATTTCAGTTGTC
TCTCCTTTGCTATAACTATTTTCCCCCAGTCCTGAATCTCTCAGTCTCCCCGTTTCTTGTGCTTGTCTCATTGTAGACCACTGCTTTGGATTGG
CAATGGGAAGATAGAAGAGGGGCTACGCTGGTAGCTAGGGTCCAGGGCTCCCATAGCCCCCAAACTCCCAGTTCTTGACTGGTCATATCTCCTA
TGAGATGGCAGGGTCTTACTTACACTGTGGTAAGGTTCCTGGGTTAGGACCAGCAATGGGTCAGGTATGGGGCAGGAGGTATGACTGGGGTCAT
CCATGCGGTCAGTGTACACACGCACACGGATGTGGCTCTTGGAGAAGGGAAGAGGTAGGGAGGCCTGTGGTTGCCTGCTAGGCCAGGGCCTGAG
TTCCCACCGTGGCTTGGCCTAATCTCCCTACCACTTGCTTTTCTGGGTGACCTATGTTGGTTTCCCCCTCTCTGGTCTTAGGGCCACTGAAGAG
TGATATACCACAGAGTACAGAGGAGAAGGTGTTGGGTAGGAGCAAAGGCTGTGGACCCAAGTGTTACTGCCAGGCTGCCATTCCCATGGATTTG
CTTTATCTCAGACAGTAATACCCTGCCCTGTAGTGCTCTGCCTCTCCACGGTGCTCCCTGTACTCTCTTGAATCATGCCATGTGACCTACACCA
CAGGCAATGTTGCTTTCACCGTCATTTGCTGCCAAGGGAACTGAGGTTGAGAGTGACTGGAAGTGTGTGTGCCCCCACATTTTCCACCTCTCTT
CCAGGCACTCAGGTCCCCTCCTGTTCTCTCTTCCCCACCAAGCATTTAACCCAGCTTGTAACTTACAGGAAGCCATTGAACAGAGCATTTGATA
CCTGGTGGCTGGAGCTTACTGTGCAGTTGGCAGGTAGAAAGTATGTCTGGAGGTAGGACAAAGAACTGAGTTTTAATGGAAGGATTTCAGGCAC
TCATATGGCCAATCCCTTAAAGAGACAGCTACTTAATCTGACCCAGCTCTGGGCTGGCTTGAGAAGGTCATAGTTTAAGGATGATGGAATACTT
TGGGAAGTGATGGGCAAATCATGGTCTAACCATTCCATATCAGCAGACCTATGTGTGGTTATAGATTTTGGACATCTCACATAGCCCTGGTTAG
CCTTGAACTCATGATGTAGCCAAGGACAATATAGAACTCTTGATTGTCTTGCTTCTACCTCTTAAATGCTGGAATTACAGAGACGTAGCAGCTC
TCATATGACAGATGCTAGGCACAGGCTGGACATGAGATCCTATGTGAGTTTGCTCTCCATGAGGCACTTCCTCTGTCCCTAGGGTGGGAGACAG
GTCACAGATGAGATGGGACTGTTACTCTAGATCTCTTTTCTAGCCTGCTTCAACTCTCGACTATCCACTCAGGAAAATCCAGAAGTCAGTCAGT
GATTGGGAGAAGAGCCAGCTCCCCCACTCCCACCCTACCCCTTTCGCCCCCTAGTCTTTGGACTTGTTGATGAAAAGATCTGGGGTGGCTGGAA
CCCCAAGCTTTACCTAGCTGCTGTCCCTTCTCCCTCCCAGGTCCCCAAAGAACTGCAGATTGAACCTGGGGCTTCATGCATGCTGCTTTACCAC
AGAGCTAAGTCCTAGCTCTTTTTCAGTTTTTTGTTTGTTTGTTTGTTTGTTTGTTTTTCAAGACAGGCTTTCTCTGTGTAGCCCTGGCTATCCT
GGAACTCAATTTGCAGACCAGGCTGGCCACAAACTCACTGAGATCCATCTACCTCTGCCTCCTGACTGGTGAGATAAAAGGTATGTGCCACCAC
TGTTGCCTTGGACTTTAGTGTCCAGGAATCAAGCTCTAACCAACAAATGTGTGTATGTGCACTGGTATAGACAATATACCGCCAATCCACAGGC
CACAGAGACCAACCTACCTTCTTTTCTCCTTCCTTCCTTCCTTCCTTCCTTCCTTCCTTCCTTCCTTCCTTCCTTCCTTCTTCTTGAGCAGTCC
TGGCTAGCCTGGATCTTGATATGTAGATCAGGCAAGTCTTGAACTCAAAGTAATCTACCTACCTCTGCCTGGCTAGTCCTAGGATTAAAGGCTG
GCCTCAAACTGACATCATTTCAACAAGCCACATATACTTTTCTTCTGTGCACACACACACACACACACTCAAGTATGCCTCCAAACATGTCAAG
AAGATAACAGATAATGAACTGTTCAAGAGAGTGGGGTTGTAGACCTGGCACTCAAAGAAACTAGCTGTGTGAGCCTGTGCTAGGTAGTTAGGTC
TTCATCTGTGAGAAGAACAGGTGGATGGAGTGTGCTTTAATCTCAGCCAGTTTTAACAGTCGTGTCCTGTGCTCATACACATAGACCCAGATGC
CTTTGTACATCCTCTTACACACTTACACAGAGCATTGCCTCGTCTTGGTTCTGGGGTCATCAGTCTACAAATCTTATTTGGGCTTTAAGACAAT
CAGCAAAGTTGGGTTGAGCCCTTCTCTAGATTTCATCTGTTTCTTTCTAGTCTCTCTGTACCTGCTGTGTTGGTTCCCCTCAGTTCCTGTCCCT
GTGTAGCCTTCACTTTCCTCCAAGAGTGACTAACATCTCTGTCTAGTGCTCAGTGGCTGTGCCCCATACTCTGTTCTGGGAGCCAAACTTCAAT
GGGGGGAAGGAGACAAAATAGAAGCCCCCTGGGCGGGAAGCAGGTGTTACGAAGCAGTAAGGCCTCCTCTGGCCCTATACAGGATGAGAGGAGG
GCACTGAAGATGTTCAACTTGAAGGACTGAGCAGGTGGCCTCTTTGCAGCTGCTCTCTGTCTCCATAAAAGCTGATACAACTCCCAGCTGCTTT
GTGAACAAAACACAGCCTCCTCAGACCCTATGCTGAGAATCATTTACACTTTACTTCCCCTTCCATAATGCAAGAGGGCGTGTTGGAGCTCACT
GAAGAAGTGGGGGGAGCACCATACACCTGCGTCTGCAGCCCAGGCTCATGGTAGTGAAATCCAGAAAAAAACATTATGGGCTAGCTTCTCTTTC
TCTCTTCCAGCACAAATTGAAGTGATCCCTTGCAAGATCTGTGGGGACAAGTCATCTGGGATCCACTACGGGGTTATCACCTGTGAGGGGTGCA
AGGTGAGTTGTACATATTTGTCTGCATACATGCACTTGGCTGTTTCAGCGGTCTCCCCAGGGTCAGGAACAGGAGGGAGGAGGAGGACCTAATC
TCGATGTAGGAATGTGATCACAGGGTCCATCACAATTATACAGTGGAGGTTCGGGGACTTTGGTGGATGTAGAAATTCTTGAGACCAGTGCACA
TGAATTGGAGGTCCCTGGGACCACCTCAAACTCCGAGAGGGTGGGATAAGTCCAGAAAACCTTTACCAGCATCATCTTAGTACTCTTGTCCCTT
CTTCCTCTAATGAGTTCTCTTCATTCTTCTTTCTCCTGGCCTAGTTCCTATCCAGTTCCCACACTGTTACCTCCTGCCTTGTTTAGCTCCATCT
CCCTCCTGAGTAACTATCCTTAGGCATAGGATTGTGGCAAGGAACACCGTCTGGGTTGGCTTCAGAATGACCCAGAAGGTGGGGGATCCAGTGT
GGTGTTCCTCGAGCCGTATCCACCTCCCTTCACCCACCTCCTCACCCACCGTCTTCACCACAGGAGGAGCCCTGGGTGGAGTGGGGGGCATGAG
GTGAGGAAGACCCAGAAGGAGCCTGTCAGCACTTTTCAGTGCTCAAAATAACAAAGCCAAAGCGAAAGGAAACATGCAGGGGTGCAAAGGGGCA
GGCGGGGCGAAGGGCTGTGCCCCCACACCTGGGAGGGGTTGGGGGAGTGAAAAGGCAGGAAAGAGAGAGCAGAACAGGATGTTCAGAAGCAAGC
CACCAGAGCCTGGGTTGGGCTGTGGTGAGTATCTAGGTCACCAGGGAGCCTGCAGGCCTGACCACAGGGAAACCTGTGTTCGCAGCCCTTCTCT
TCCTCCAGCCCTCCCAGACAGGCAAGCTGACCCCAATACAGCCTGAGGCCCCTTACTCAACCCCCACCTCAGCCCTAGTCTCAGGAGACTCGAC
TCCCCGGCCCAAGCCCATTGCTTGAGTTCTGCACTATTAGCATCTAGTCTAGAGCTGAAAGCATTTTGCTGGGGAGATGGAAATGCTGGTGGAA
CCGATCCTAAAGAGAGCAGAGCCAGGCATGAAAGGAGCCTGGAGCCTAAGCTCCCCAAAAGAAAGCTCATAGACAGAGCTGGGGAAATGGAGCA
TGGAGTCAGATTCATGTGCCTGTTGCCCTGTCCTGTCTTGTCTTCTCTTACCTCCTGTACTGATTCCTGAACCTTCTTCAGCTGTCAAGTTTGG
CCGAATGTCCAAGAAGCAGAGGGACAGTCTACATGCAGAAGTGCAGAAACAACTGCAACAGCAGCAGCAACAGGAACAAGTGGCCAAGACTCCT
CCAGCTGGGAGCCGCGGAGCAGACACACTTACATACACTTTAGGGCTCTCAGATGGGCAGCTACCACTGGGCGCCTCACCTCACCTACCCGAGG
CCTCTGCTTGTCCCCCTGGCCTCCTGAGAGCCTCAGGCTCTGGCCCACCATATTCCAATACCTTGGCCAAAACAGAGGTCCAGGGGGCCTCCTG
CCACCTTGAGTATAGTCCAGAACCAGGCAAAGCTGAAGGCAGAGACAGCATCTATAGCACTGACGGCCAACTTACTCTTGGAAGATGTGGACTT
CGTTTTGAGGAAACCAGCCATCCTGAACTTGGGGAACCAGAACAGGGTCCAGACAGCCACTGCATTCCCAGTTTCTGCAGTGCCCCAGAGGTAC
CATATGCCTCTCTGACAGACATAGGTGAGCATCTGGGAAGGGTGGGGGCAGTGAAAATGAGACAAGAACTTCCTCCAGCACGGTGCCCATGTAA
TCAAGCATTAGTCCTAAGGAATTAGGGATCCTGGACAAAAAGCCAAGGGAGGAGGCAGAGCAAGGGAGGCCAGGCAGAGGGCCTCTTTATGAAC
TTTGGGGTGGGAACTGGCTGATATCTAGCTGTGACTTCATCTTCTGGCCCCAGAGTACCTGGTACAGAATGTCTGCAAGTCCTTCCGAGAGACA
TGCCAGCTGCGACTGGAGGACCTTCTACGCCAGCGCACCAACCTCTTTTCACGGGAGGAGGTGACCAGCTACCAGAGGAAGGTAAGGGCAAGAG
ACATGAGGGAAGGGAGGGCACCACCACACGCGGGTGCGGTGCGGGCGCGCGCGCGCGCACACACACACACACACTGGTAGAGCCCAGATATGGC
GTCTTTCACAGGAATGGGGGTAGCAATAGGGTATCACAGGCTGGCTAACAGGTGTGACTGTCTGTATCCAGGCAGCAAGATGACTATTTCTGTA
GCTCCTGCTTATTGAGATTTCACCTAGTTAGCAACCTACATCCTCTTCCACCCAGACCTTGCCACACTGCCTTTCATCCTCCATCAGTAACTGA
TGCTAATCAGTATCTGAGGGTCATTTACTGGACACCCTTTCCTGTCAGGCATTGTAATAGAAGCTTTGAATTGTGTTCAACCTGCTGTTTGTGA
ACTACAAGTGTCTAAAGATAGTGATAAATGTGGCCCAATGCAAAATCATGAGAATGTTTTTGGTTGGTTGATTGGTTGGTTGGTTGGTTGGTTG
GTTGGTTGTTGGTTGGATGTTGTTGGTTATTTGGTTGTGGTTGGTTGTTGGTTGTTGGTTGGTTGGTTGGTTGGTTGGTTGGTTGATTGGTTGT
TGGTTAGTTTGGCTGGTTCCAAGACAAGGTTTTGCTGTGTAGCCCTGGCTGTACCTGGAACTTACTTGGTAGACCAGGCTGGCTGGACAGGGTC
TCTCATAGTCTAGCCCGGCCTATGAGTGACCTAGGTCTGCTGAGTCCCAAGAGATGATTTGCCTATCTCCACATCTCAAATGTTGAGATTATAA
ACATGTACTACCTTGTCTGGCCTCTCTCTCCCTCTCTCTCTCTCTCTCTCTCTCTCTCTCTCTCTCTCTCTCTCTCTCTCTCCTTCCCTCCCTC
CCTCCTTCCTTTTCCTTTTCCTTTTCCTTTTCCTTCTTCTTCTTTCTTGTTTTTTTTTTTCTTTTGGGTTTTTGAGACAGGGTTTCTCTGTGTA
TCCCTGGCTGTCCTGAAACTTACTCTGTAGACCAAGCTGGCCTTAAACTTAGAGATCTGCTTGTCTCTGCCTCCCAAATACTGAGATCAAAGTC
TTGTGCCACCACATCCAGTTTTCACCTGGTTTTCTTTCCGTGGGTTCTGGGATCTGCATTCAGGCCTTCATGCCTGAAGGCCAGCACGTTGTTG
ACCTTCATGGTACTTTTGGTAAATGTGAGGTCAGAAGCTGGGGTAGACTGATAAAGCAGAAGAGTAGCAGAAGGCAGCCTGGAAACCGCTGGAG
GAAGAGCAGTTAGGAAGGCTTTCAGTGCCGTATGAAGAGTGAAAGCCTCACTTCTAGGCAATGCTGTACTCAGAGCTGCTTAGGGAATTTCCTT
CTGACCTGTGTGAAGGATCCATCTATGTAACCGTTCGCTCAGGCCCTTGTTATTCACAGTCATCACAGGAGTCCTGAGATACAGAAATCACTTC
AAACAGTCTCTCCCTGAGGAATGTAAACTTTAATCTACAACAAAATGGATTTAAAGAAGGTCTGGGAAAGCAGTTGGAATACAAATGTGGTTGG
ATCCACTTTGGTGCCTTTTGTTCTGGGTTTTTGAGGCAGTTTCATGAAGCCAAAGGTAGCCTTAAACTCATGATCCTCCTGCCTCCAGCTCCTA
AGTGCTGAGATCACAGCCATGTACCACTTTGCCCGGCAGAATCCACTTTCAAAATATATCACGTGTCCCATGATGTGCCCCCCCCCCATCCAGC
TATCAGCCACAATTGGACTATTATACTCCTAACTCAATCCCACCCCATCTTTGTCTGCCACGTCAGAGCCACACAGCACTTGCTCAGTAACCAC
CCTGGTCATTTTAAGGAGACAAGACAAAACCTGCCTCTTTTGATCTCCAGCCTCCTATTTTAGCCAAAATCCAACTCCAGAGTCCATGTTACAG
TCAAATGGACCACCCTGCCTTCCTCCTCACCTGACGTTGCACCATCCTTTGACTCCCCTACTCTGACCACAGTATTTTTTCACTGGTCCCATCT
CTCTGGGGCCTCTTATACCTGCTATTGTCTCCTGTCATATTCTCTTCCCTGTTTCCAAGTATTTTCTCCAGGCCTTCATTCAACAGAGGTTTTC
CTTAAACAGTTACCGGACTGGAGAAGGACCCCATCCCTTATCCTGCTTTAGTATCAGATACAATCATATGCTGCTTACTAACAGGAATATGTTC
TAAGAAATGCTTACCCATAGCTTCACATGGTGGGGTACAACTGGATATACTTAATACAAACAAACACAGCTGTACTGTCGTCCATACTCCCTTA
GCCTTATGGGACCACTGAAGTTTGAGGTTCATAATTCCCCGAAAAGTTGTTAGGCGGTACGTAACTGTGTTTATGACTCCCTGGCTGCTGTGCA
CATTTAATAAAAGCACATCTTTTTATTCACCGATGTTACACCAGAGCTCATGACAGTGCCTCATCTAGATTTATGGAGTTTCTCAACAAATATC
TTTTGAATGAATAAAAGAAACTATTGTAGTAACCCAGGCAAACTCAAAGTACTAAAACACAAAATAAATTCAAGAGGCGTAAGGGACTCACTCT
GCTACCTACTTTTCTGATTGTTACTTTTTCTAAGGAACTAAGCTAAGAAGGGGAGATGTCAAGAAAGAGTGGTTTTTAAGTCTCAGTTCATTAT
GCTGTAAATTGCGTAGGAGAGATGGTGCCTGGGGTCTTTCTCCCTGCCTCTGTCCCCTGGGCTTCTGCACCTTTCTTAGAGGTTTACCTTCCTT
TGGCTGGAGGCTTACTATACTGACTTACTACCTGCTGAGTTAGGCACCTTCCTAGAAACTGTTTAGTTAAAACAAGCAGGTGATGAACATGAAT
TCAGCATTCATGCCAGGGACAGAAGATACATAGTGGCAAAAAGACAGATGGACTTTATCTAAGTACATGTGGGGCAGGAAGGCAGACAGACTGG
ATCCTTTTTTCTACAGTGTGGCAAATGCTACACAAAGGGAAGCCAACAGGACTTTTGCCCAGCAAGGGATGGGGATGATATTCTCAGAGGAGAT
AACACTTGAATTAGCAAAATAAGAGTTAGCAGAGCCTCACCCTCCAGATGAAGAAACAACATGAACCAAGGCAAAGGGCCATGGGAGCAAACAC
AATTCATTCTGGGGTAGGCAGAGCCATTGATGCCATCATTCAAGCATGTCCTTGCTCCAAGCACAGTCCTGAAGGTACAGAAATGACCATCAAA
ATTGAACCTAGGAAAAGCTCACCTGTCTCGTGTCAGTTCTAACTTAGCCTGAGGTTTCCAGATATGCATACATCTAACAAATGGGATTGGCTGC
TCAAGTTGGGTCAAGGGTCGGGGAGGCATCAAGAAGGGTCATCCTAGCTCAGTAACAGTGAGATGTGTTTGCTCTGCTAAGCACCGAAGTACTC
ATTCCCTCCTAACCTATCCTCAGCTGCCACCCAAAGGAAAACTCCGGAGCCTGTGCAGCCAACATCTGGAAAAGCTGCAGATCTTCCAGCACCT
CCACCCCATCGTGGTCCAAGCCGCCTTCCCTCCACTCTATAAGGAACTCTTCAGCACTGATGTTGAATCCCCTGAGGGGCTGTCAAAGTGATCT
GGAGGAAGGACAACCTGAGGTTTTAATTCATACAGGACACCAGAATTCATCCCAGCTCCAGCTGTCCTCTGTCCCTAAGAGAAGCAGAGGACCG
GATACTAACCAGCCGGAAAACCCAAGGACCAGCACCCGGGAAGATGCCCTTGACTTCAGTCTTTACGCTATGAGGAAGGAAGGCAGCGCCATCC
CCTGCTCTGTACCTGTGTGCTGCTGACTCCACATGATGGAGAGACTAGGAACAGGACAGGGACCTGTTTCTCCTCCATAGTCTTGCTCAGAATT
TCTCTCAGTTTTGTAAAGCTGCAGACTCTCCTAGGAGGTATAAGCAGCAGATGAGAGGGAGGGAGGTTTTTTTTTTTTTTTTTTTTTTTTTTCT
CTCAGAGGAAGGGTTTAGCCAAGTAAACATAAATCCCAACTTGTGCCATTCTTTATAAAACGATTTCAAAGGCTGGAAGTGTATCGTGCCTGCG
TGCTTGTACAAGGGTGCAGGGCACACATGTCGGGTGTTGGGAGACTTGAATGTGACTGCCTAGGATACATGCTTGCCCTGCAGTTTTGTTTCTG
TGTCAAGCCAGCAATTTATCTGTTTTATAAGAATTTTAGCACACACACATACACACACACACCGCCCAAGATTCTCCCTCAGCTAAGCAATCCA
CCAGGGACACTGGTGCTCAGATACCTGACACAAGAGAAATGGCAAGCTAAACTGAAGGAAGGTATTCTAGACTAAGAACTTCCAACAAATGATA
CCCACAGGCCCGTTTAGATTTAGAAATTGCACAGAATTGCCCTGCATCTAAAAGACTAGAGGCTGTGGAAGCGGTGTCCCGGGAGCTCTCTAGC
TCCCCAGGAGGAACCAGGATGTCAAAACTCTCTCAAGCTTCCCAGGCTGTCTCCGTAGCAGACCCTGCCCCAGCCCTGGGTCCCCTCTTCAGTT
CCTCCCCCGATTCGATTCGTCATCCCGTAGTGGCGCTTGCTGCAGCCCTCCCTGGTTGCTTTATTTATTTATTTTGCACCAACAGGGTTGCTGC
AGACTCATTCTGGTTTAAAAAGAGAGAAGAGGAGGGAAAAAAAAAAAAAAAGAAAAAAATGCTTCCTGGCTCTTTTCTCTCCTTTGCTCTTGGC
AGCGCGACCGCAGTAGCGGCGGCAGCAACAGCAGTCTTGCCAGCCGGCTGATGCGGCAGGCTCCCGGGCAGTGGGGAGTGGGGACTCAGACACA
CGGGGAAGGTGGAGAGGCCAAGGTGCAGCTCGGATGGGACAGGCCCCACCCCTGGAGAGATGCAGCGCCCAACTTGATGCCACCCCCCAGCTTC
TCCGGTAAGTGCCCCTGCCCCTCTGTGGGCACGTCTCAGCTGCCCTTTCCCATGGCATTCTCAAAGACAAGGCCATGTTAAATAGCCTACAGGA
AAGAGAGCTTCCCCTCTACCCTAGCTGACCATTCATCCTGTGATTGGAAAACTAAAATGTCCCAGGTACCCCTGGTAGGGAGAGTCCAAGGAGC
CCCCCCCCCCCGCTTTTTTTAGCCTCTAAAAAGCTGCCCTCCTAATCTGTGTGGATACTCCAAAAATCTCTCTCCTAAGTGCCCCTTCACCATG
CAGGTCCCCATGCCTCAATCTGGCATGCTTTAAAGTGGATATGTGCTCGAATGAATCTGTGGCTGCCACATAGACAAGAAAGCTCCATCTCCCT
GAGGGGGCAGTGCCCCAGACTCTAGACCCCAGGGGAAGAGAAAGAGTT
MOUSE SEQUENCE - mRNA
GACGGGCCAGGTGCTCCCTCCCTCTTCCCTCCTCCCTCCCTTGGGCCCTGCTCCCTGCCCTCCTGGGCAGCCAGGGCAGCAAGGACGGCACCAA
GGGAGCTACCCCATGGACAGGGCCCCACAGAGACACCACCGGACATCTCGGGAGCTGCTGGCTGCAAAGAAGACCCACACCTCACAAATTGAAG
TGATCCCTTGCAAGATCTGTGGGGACAAGTCATCTGGGATCCACTACGGGGTTATCACCTGTGAGGGGTGCAAGGGCTTCTTCCGCCGCAGCCA
GCAGTGTAATGTGGCCTACTCCTGCACGCGTCAGCAGAACTGCCCCATTGACCGAACCAGCCGCAACCGATGCCAGCATTGCCGCCTGCAGAAG
TGCCTGGCTCTGGGCATGTCCCGAGATGCTGTCAAGTTTGGCCGAATCTCCAAGAAGCAGAGGGACAGTCTACATGCAGAAGTGCAGAAACAAC
TGCAACAGCAGCAGCAACAGGAACAAGTGGCCAAGACTCCTCCAGCTGGGAGCCGCGGAGCAGACACACTTACATACACTTTAGGGCTCTCAGA
TGGGCAGCTACCACTGGGCGCCTCACCTGACCTACCCGAGGCCTCTGCTTGTCCCCCTGGCCTCCTGAGAGCCTCAGGCTCTGGCCCACCATAT
TCCAATACCTTGGCCAAAACAGAGGTCCAGGGGGCCTCCTGCCACCTTGAGTATAGTCCAGAACGAGGCAAAGCTGAAGGCACAGACAGCATCT
ATAGCACTGACGGCCAACTTACTCTTGGAAGATGTGGACTTCGTTTTGAGGAAACCAGGCATCCTGAACTTGGGGAACCAGAACAGGGTCCAGA
CAGCCACTGCATTCCCAGTTTCTGCAGTGCCCCAGAGGTACCATATGCCTCTCTGACAGACATAGAGTACCTGGTACAGAATGTCTGCAAGTCC
TTCCGAGAGACATGCCAGCTGCGACTGGAGGACCTTCTACGGCAGCGCACCAACCTCTTTTCACGGGAGGAGGTGACCAGCTACCAGAGGAAGT
CAATGTGGGAGATGTGGGAGCGCTGTGCCCACCACCTCACTGAGGCCATTCAGTATGTGGTGGAGTTTGCCAAGCGGCTTTCAGGCTTCATGGA
GCTCTGCCAGAATGACCAGATCATACTACTGACAGCAGGAGCAATGGAAGTCGTCCTAGTCAGAATGTGCAGGGCCTACAATGCCAACAACCAC
ACAGTCTTTTTTGAAGGCAAATACGGTGGTGTGGAGCTGTTTCGAGCCTTGGGCTGCAGCGAGCTCATCAGCTCCATATTTGACTTTTCCCACT
TCCTCAGCGCCCTGTGTTTTTCTGAGGATGAGATTGCCCTCTACACGGCCCTGGTTCTCATCAATGCCAACCGTCCTGGGCTCCAAGAGAAGAG
GAGAGTGGAACATCTGCAATACAATTTGGAACTGGCTTTCCATCATCATCTCTGCAAGACTCATCGACAAGGCCTCCTAGCCAAGCTGCCACCC
AAAGGAAAACTCCGGAGCCTGTGCAGCCAACATGTGGAAAAGCTGCAGATCTTCCAGCACCTCCACCCCATCGTGGTCCAAGCCGCCTTCCCGC
CACTCTATAAGGAACTCTTCAGCACTGATGTTGAATCCCCTGAGGGGCTGTCAAAGTGATCTGGAGGAAGGACAACTTTCTATTTCCTTCAGCC
CTCTGACCCGTCTCCCTGGACTCCCTTCACCCAGCCTTTCCCTTTCTGCACTCTATGAAGGGTGGTATCCCTAGGAGTAAGCAAATCCTAAGAC
TGATTTTCTGCCCCTAGGCTTGCCTTGTAGGACAACAGCAGCAAGTGATGGAGAAAAGGCTTGTTATGTTTGATTTCCCATAAGTTCCACCCTG
GCTTCTGGAAGCTGTGGGGTAGATGGGATAGAGATAGGATGACCAAGTCAAATAAAAAACAGACTGACAATCAGCAGGGATAAATCCAGGTACC
TGGGATAAGGAGAACTCAAATCTAGGCTTGAAAGCTAATAACAGTCCTTTCAATACCTCATTGTATTTCCCCATGGGTCCTCCTGGGGGGACAT
GGATCTAGCTCAGAGACTGGTGGCAAGCCCCCAGAAGGACCTGTATATAATAAGAATATAGATTCCTGAGACTTTTCTGCCTTTCTTCTTCCTA
GTTAAGAAATGTTGTTGACCCCCTCTGCCTGTTTTCTGGGACCTAAAATGCCTGGATGTGTAAAGAATGAGGGTGGGGTGGAGATAAGGTCCCA
AGATAACTGTTTTATGGGGTTTGGGTATGAAGAAAAACATCACTGGAAAAATTAGAATGGAAACCTCTTTGCACACTTTAAAAGTGTCAGATTC
GTTAGCAGTCTAATCAGAGACACACATCCACACAGGTGGAGCACACAGAGGCTCTGCCCCCAGTGACACCATTCTGTAGACTTTCCCTCTGGCA
CACAATCTCTTCCTTGAGGTTGCAGCTCTGAGAAGCCTGAGGTTCTAATTCATACAGGACACCAGAATTCATCCCAGCTCCAGCTGTCCTCTGT
CCCTAAGAGAAGCAGAGGACCGGATACTAACCAGCCGGAAAACCCAAGGACCAGCACCCGGGAAGATGCCCTTGACTTCAGTCTCTACGCTATG
AGGAAGGAAGGCAGCGCCATCCCCTGCTCTGTACCTGTGTGCTGCTGACTCCACATGATGGAGAGACTAGGAACAGGACAGGGACCTGTTTCTC
CTCCATAGTCTTGCTCAGAATTTCTCTCAGTTTTGTAAAGCTGCAGACTCTCCTAGGAGGTATAAGCAGCAGATGAGAGGGAGGGAGGTTTTTT
TTTTTTTTTTTTTTTTTTTTCTCTCAGAGGAAGGGTTTAGCCAAGTAAACATAAATCCCAACTTGTGCCATTC
MOUSE SEQUENCE - CODING
ATGGACAGGGCCCCACAGAGACACCACCGGACATCTCGGGAGCTGCTGGCTGCAAAGAAGACCCACACCTCACAAATTGAAGTGATCCCTTGCA
AGATCTGTGGGGACAAGTCATCTGGGATCCACTACGGGGTTATCACCTGTGAGGGGTGCAAGGGCTTCTTCCGCCGCAGCCAGCAGTGTAATGT
GGCCTACTCCTGCACGCGTCAGCAGAACTGCCCCATTGACCGAACCAGCCGCAACCGATGCCAGCATTGCCGCCTGCAGAAGTGCCTGGCTCTG
GGCATGTCCCGAGATGCTGTCAAGTTTGGCCGAATGTCCAAGAAGCAGAGGGACAGTCTACATGCAGAAGTGCAGAAACAACTGCAACAGCAGC
AGCAACAGGAACAAGTGGCCAAGACTCCTCCAGCTGGGAGCCGCGGAGCAGACACACTTACATACACTTTAGGGCTCTCAGATGGGCAGCTACC
ACTGGGCGCCTCACCTGACCTACCCGAGGCCTCTGCTTGTCCCCCTGGCCTCCTGAGAGCCTCAGGCTCTGGCCCACCATATTCCAATACCTTG
GCCAAAACAGAGGTCCAGGGGGCCTCCTGCCACCTTGAGTATAGTCCAGAACGAGGCAAAGCTGAAGGCAGAGACAGCATCTATAGCACTGACG
GCCAACTTACTCTTGGAAGATGTGGACTTCGTTTTGAGGAAACCAGGCATCCTGAACTTGGGGAACCAGAACAGGGTCCAGACAGCCACTGCAT
TCCCAGTTTCTGCAGTGCCCCAGAGGTACCATATGCCTCTCTGACAGACATAGAGTACCTGGTACAGAATGTCTGCAAGTCCTTCCGAGAGACA
TGCCAGCTGCGACTGGAGGACCTTCTACGGCAGCGCACCAACCTCTTTTCACGGGAGGAGGTGACCAGCTACCAGAGGAAGTCAATGTGGGAGA
TGTGGGAGCGCTGTGCCCACCACCTCACTGAGGCCATTCAGTATGTGGTGGAGTTTGCCAAGCGGCTTTCAGGCTTCATGGAGCTCTGCCAGAA
TGACCAGATCATACTACTGACAGCAGGAGCAATGGAAGTCGTCCTAGTCAGAATGTGCAGGGCCTACAATGCCAACAACCACACAGTCTTTTTT
GAAGGCAAATACGGTGGTGTGGAGCTGTTTCGAGCCTTGGGCTGCAGCGAGCTCATCAGCTCCATATTTGACTTTTCCCACTTCCTCAGCGCCC
TGTGTTTTTCTGAGGATGAGATTGCCCTCTACACGGCCCTGGTTCTCATCAATGCCAACCGTCCTGGGCTCCAAGAGAAGAGGAGAGTGGAACA
TCTGCAATACAATTTGGAACTGGCTTTCCATCATCATCTCTGCAAGACTCATCGACAAGGCCTCCTAGCCAAGCTGCCACCCAAAGGAAAACTC
CGGAGCCTGTGCAGCCAACATGTGGAAAAGCTGCAGATCTTCCAGCACCTCCACCCCATCGTGGTCCAAGCCGCCTTCCCGCCACTCTATAAGG
AACTCTTCAGCACTGATGTTGAATCCCCTGAGGGGCTGTCAAAGTGA
HUMAN SEQUENCE - GENOMIC
CTTCTGGACTTCTTATTATGGAAGTCAAGTGTCCATATTGTTAAGTCAGACTGAGTTGGGTTTTCTGTTTTCTCACACTTTTAGCGGATGTCAG
CCTAAATGATGCCCACGTGTTATCTTTAATCCTCATACCAACTCTGAGAGTTTTATTCTTTCTGTTTTCCAGATGGAAAAGGTAAGCCCCAAAG
AGATTAAGTTACATGCTAGAGGTCACACAGCTAGTCAGTCGCAGAATCCAGATTCCCAGCCCATCTGACTGATGACAAAGCAATGAATACTGCA
GCACTCCAAGAACATGGACCCTCCCCAGAAACAACACCAGGGCTGAGCTGAGTCAGAAGCCACATGAATGGAAGGTCTGGAGGCCACCAGGGAT
CGCCACAGAAGAAACACTTGCTTTGTTAAAATCTTCAAGTATTAGATGACACAATAATCATTGCATTCACTGGTTTGTTTCATCTTTTTTCCTG
CATTTCAGGATTTGTCTTATTGTTGTTTGCATGTTTATTGTCTGTCTCTCCCCTTAGACAGCTCTCTATTCTATGTATCCCCCTAGCACAGTGC
CTGGTGCATTTTAGGCACTTCAATGAATGGATGAACAAATTGATTCAAGTATAATTTTGCTTACTACTTCACTCATCAAATACATCCTGACTGA
GTACCTGCTTTGCACCAAGCACTGTTTTTAAGATTCTTAGTCTAGTAAGAGAGGTGGACATTAAACAAATAACCACATAAATAGAGGGGGAAAA
ACTGTAATCAATGCTATAAAGGAAAAGTATGAGGTACTATGAAAGTGTACAGCAGGTGCCCCTAATTTAGATGGGAGGGTGTCAGTGAGGGCCT
CCAGAGCTGAAGCATGAAGGAGAAGAGTGGTCCTAGGGGAGGAACTAGCCTTGCTTCCTTTCAGAGACTGAAGGATGGTAGGTGTGTCTAGACT
GCAACTGATGGTCTCATCTTGTCTGTGGTGGCCTGTGCTTCCTAAGATCTGTCAGGGATGGAGGACATAGGCCAGCTTGATCCTGACTGTTGGA
TCCAGCTTTATCTTCAGTGTTTCTAAGTCCAGAAATCCTGAGTCTGGCCACTTTTCCTTTAGCCACAATAATTCAGAGAACTASTTAGCCAGAT
AATGCAGACAACTGATCACTGCAAGTCACTCTGCCTTGGAGCTATTGGAATGGCTCCAGAACAGGGCACTGGGGGCCAAGGCAGGATTTCAACC
AGGAGGGTTTTACAGAAGATGGACTTCACCTGGTGAGGGCTGGGGTTGGAACCGAGCACAAAGAAGTGGCCACTGTCCACTGATGGCCGTCTCC
CTCTTCTCTGTAGCCCTCCCCCACCCCTAAGCAGCCCAGCTTCAGCAGAGGAGGCCTGGGCACAGGAATGGATTGTTGCTAGAAACCTGGCTTC
AGCTGGAGGAAGTCAGAAGACCGGGCTGATGGCCGCGCAGACAGAGCTGTGTTTATCCAGGAAAAGAGGTAGGACGTAGGCAGCGGAGGCCCCT
GCCAGCTCCTCCCATAAGGACTGCGCTGGGGGAGCAAGCTGCAGAGAAAGCTGCTCCCCTCTGGCTCTGAAAGTGTCAGGGCCTGAGGCCAGAA
CCCAAAAGCGACCCACAGGTGAGGCCAGCACTCCGGGCGCGCGACGGAGAGGGTCGCTAGCTCGGATCCCAGGGCAGAGGGTCGGAACACGGTG
CTCTTGGTGGCTCCGCGCAGGGGTGAGGCGAGCGCGTGGTGGCGGAGCCTTCCGGAGACCGCTCTGAGGGTGGCGGAGAATCAATGCTGCCTTT
GTGCTCGGTGGTTTCCGAGGGAGAAAAGGAGTGGAGAGTAGGAGGGGCGACCCAACGGCCCGGCCCCGCCCTGCAGACGCGGGATGCGGTAGGG
CTGGGAGGCGCGGGAGCTGGACGGTCAGGGCTACTGGGGGTGGGATTCTGTGTCCCCGGGAGGTGCGTCTCCTTGGACCAGCCCAGGGTGTCCT
GAGGTCAGCAGCCAGGTGTGTCCCCTGGCTGCCAGGAGCAGAAGTGAGTCCTCTTCTGGCTTGGAGCGACTCGAGGAGGGGAAGGAGGAGCCAG
AGGAGGCAGGTGGGGCTAGGACTGAGGGATGGCTCAGGCCAGAGAAGCTTGAGCCGGGGCAGCCTGGCAAAGGGAGGAAGTCCCAAGGGGCGCC
AGACTGGATCCCAGAGGAAAACCCAAGTCTTCGAAATGGGCGGGGGAGGAGGGCGTGAGCCCGCCTAGGGCGCAGTGTCCCAGGGGGGTTGGGA
TCTGAGTCCTTTTGCTATAATGCGGTTATTATCCTATTTGTCCTAATAGTACCCTAGGGTTCTGGTGGATACTATATGGGCCCTCTAACTGTAA
GGTGAATGGGGCATCTTTAGTACCAGTGGGAGAATCTGTGTACTTGGTGACGGAGGCCCGTATATGAAAATGTGACTGACTGGCCCTTTGCTCC
CTCCATTTCTCCCCAGCCCCCCAGACTGCGCCGCCCTTTCTTTTTCTGCTCAAATAGGGGTCCGCGCTCCCAGTGGGTCCGGAGACCGGGGCGC
GGTGGCTGACCGCCCGGTGTACGCCAGCATCACTCACCTCTGACTCCTCTGCGCTCCTCGCCCAGCCCCGCTCCGCCGAGCACTCCCATCTGAG
ACGGGATTTGGACCCTCTCGGCCCTGCGGAATTCGGAATTCCATTACCCCACCCACCGCATGTGGCTCTTGGAAAAAGCTGGCTATAAGGTGGG
GGCCGCGGATCCTGCGGCCCGTTGGGCGCCTTCCGGCCTGTTCTCCAAGCGTCGCGCCCCGGGCCCGCCCACAAGCGCCTGCCCCAACGTCCTC
ACCCCGGATCGCATCCCGCAGTTCTTCATCCCGCCTCGGCTCCCGGACCCGGGCGGCGCATTGCCCGCGGCCCGGCGGCACGTGGCGGGGCGCG
GCACTCCCCGCGACCTGTTCGCTGCCTCACCTGGCGGGCCGCGAATGCTGGGCCTTCCTGCCCGAGAGCCCGCACACGCGCCGGCGGGAATCCC
TGTTCCACGGGCCGCCACCTGCCCCGGCCGGGGGACTCCCCGCGGCGCAGTCCCGGCTGCACGTCTCCGCCCCGGACCTGCGCCTCTGCCGGGC
CCCCGACAGCGACACGGCCTCGTCGCCGGACTCGTCGCCCTTCGGCTCCCCGCGGCCAGGCCTGGGCCGGCGCCGGGTGTCCAGGCCTCACTCT
CTGTCCCCAGAAAAAGCGAGCTCGGCCGATACCAGCCCGCACTCGCCGCGCCGCGCCGGGCCGCCCACGCCGCCGCTCTTCCACCTGGACTTCC
TGTGCTGCCAGCTGCGCCCCACGCGCGAGAGCGTGCTGCGCCTGGGGCCCCGCGGCGGGCAGCTGCGGCTCTCCACCGAATATCAGGCCGGGCC
CGGGCGGCTGCGGCTGCGCCTAGTGAGCGCCGAGGGCCTGCCCCGGCCGCGGTCCCGCCCCGGGAGCGGCGGCGGACGAGGATTTTTTTTTCGA
CGGGCTCGGCCCCCCGGACCTGGCCGCCCGCAGCCTGAGAGCCAAGGTGCTAGACAGGGGCGCGGGACTTCGCAGGGATGTGCTGCTGGGGGAG
TGCGAGACGCCCCTCATTGCGCTGCTGCCCCCGCTGGGTGGGGGACTAGGTCCCGGGTCATCCCTGGCGCCCACCCATCTCAGCCTGTAGCCTG
AGCCCCTGGCTTCCTCAGGACGTCTCCACTGTGTCTGCAGTCCACATTCTTTCCACCCTGCCCGGCTTGTATTTATTTTTGCTAATAAAATGTC
CCCTTGTCCTTAGCCAGATATTTCCCCTTACTGGCACCTTACACGCTCGGGCATAGAGCCTACCGATCTTCCCTCTATCCCGGCCATACGCGGG
GGGAAGATGCTCATTCAGACGGTCTGAGTAGAGCAGACCTTAAGCAGTGGTTCTCAAGCTTGAAGGAGCATCAGAATAACCAGACATCAGAAAT
CCTGGAAGAGCTTTTTAAATATGCAGGGTCCTGGGCTCCATCCCCATGAGATGCTCCTTCAGTAGATCTGGGCCAAGTAGGTCCTAAGGTCCCC
GGCCAACAAGGCCCCCGAACAATCAGGGAGCCCTGCAATCACTGAGTCACTCTGACAGAAACCAACACAAGCCACCTTCCACTTCAGGCTGCAC
AGAGGAAATTAACACTCCCTTCCTGTGCCCCTCTAAAACCCACATCTGTCCTGAAGACAGATGAAATTTCTGGCTCTTGAGAGTGAGTCAGGGG
ATGTCAGATGAACACAGAGTGCCCTTTAGTCTTCCTTCTTCCCTCTTGCCTCTTCCACACCTGTGCGTCCCTTGAGGGCTGGGGCAAGTCTGGA
GCCAAGAACCAGAGAGGTGCTTGGGTACCAAATGAATGAGATATAATGTCCCAGGGATTGAAAGGGGGGAGGTGGGGTACTACCAAAGTGTGAG
AACTGTTCTCAGGAATGCCAGAAGAGCCTCCTCCAGCCAGAAGGAGGTGGGGGTGGGGGGAGGTGGATAATTGTAGGTCACATTGTGTTTACCT
CTACCTGAATGTCCCTGACGTCAGCAGTGACCTTCTTCTCTCTCCCTCCACCTTCCCAGGAGCCGGTTCCTCTGGACTCAGGCCAGGCTTGGAG
GAGGGAGGGAGGAAGTGAGGTAGGCACTAGGCTAACCCAACTTCTTTCTCCCTCTCCCTTGGGCCCTTTCTCATTTTTCATGGTGCCAGCTGTG
GCCTGCACCAGGGCTAAGCTTGCGGGATCTGACAAAACCTCAAAAGCCTCAGCACCTGGACACGTTAAATTCCAACACACACACACACACACAC
ACACACACAGAGACACACACACAGAGACACACACACACACACACACACACACACACACACACACACACACACGGCTTCATTCAGACAGCTCTCC
AGTTCCTTCTCCCTTCCCTGATTGGGTCCGCACCACCCAGCCCTAGGCCAGAGAACTCTTGCTCCAGGTGTCCACCAGGTGGTGGCATTGACCA
AACAATCCTAAGTGCTAACTACTACACATGGATTATAACCACATGAGGAACTGTTCCAACAATTAGTCAAGCCATTTATTCCTCCCAGCACCTT
CTATTAGGGTGTAAATACTATTATTATCCAATGGGATCTTGGGCCTCGCATAGCTACTAAAGGGAAGAACTAGGACTCACATAGCTAGTAAAGG
GAAGAATTCAGGGAGTCTGGCTCCAGAGTCTGGACTCTTAATCACTTCAAGATAATACCCCCCACTGCCAGCTGCTGCCTCCCAATCAAGTCAC
TCTAAAAGGGGCCTCCTTCCCTAAGATGGTAGCTCTGCAAAGGTTTCGTACCCTGCCAGTTTAAAATCCCATGTTTGAATTCTGGGAAGTCTAC
CTATAGATTTAGGTCTACCCCTAGTACTGGATTTCTATTTCAGCCCCCAAATTCTCCCATAACAACCCTTGGTGAGAAACTGGATTTGATGCCC
ATTTGAAAGTATCTGGAAAGAAAGTTAGAGGCCGTAGAACCTTCATTCATGCATTCCTTCAACAAATACTTCGGGAAGGTCCATCAGGTGGCAG
GCATTGGATACAGCAGTGATTAAACAGACATGGGGATCAGAAAAGTGATAAAATAAGCCATTAAAATGTAAAGAATGCTACTTGGTGATATGTT
ACAGAGAAAAATAAAGCAAGAAAGGGAATAGAAAAGAGGGGTCAGATAAAGAGAAGGGAAGATTATAGGGACAGAAAGAGGGAGGGAGGAGAGG
AAACTTTTTTTTTTTAACCATAGTACTTTGGACCAGGTCCTGAACTTTATCTGTGTTATCTCAGTTGATCATCACAACCACTCAGTAGAGTAGA
TATTAGTATTCCTCTTTTTTTGGGGTGGGGGGACGGAGTCTCACTCTGTTGCCCAGGCTGGAGTGCGGTGGTGCGATCTCGTCTCACTGCAATC
TCCACCTCCCAGGTTCAAGTGATTCTCCTGCCTCAGCCTCCCGAGTAGCTGCCACTACAGGCGCGTGCCACCACACCCGCTGGTTTTTTGTATT
TTTAGTAGAGACGGGGTTTCACCGTGTTAGCCAGGATGGTCTCGATCTCCTGATTTGGTCTCATGATCTCGGTCTCGGTCTCATGATCCACCCA
CCTCGGCCTCCCAAAGTGCTGGGATTACAGGCGTAAGCCACCGCGTCACAACCACTCAGGAGAGTAGGTATTATTATTCCCACTTTACAAAGAA
AGAAATGGATCAGATAGATAGGTTAAGTAAGCTTGCCTGGGGAACAGCCCTGGCTGGAGGGAAAGGTTCTGACCTGACTCCACCCAGACACCCC
CACTGCCACCCCAACTGCTTAGGGGAAGCTGTGAGGAAATCCAAGGCCACTCCTCCCAGGAGCAGTTCACTCAGCATTCACAGCCCTGAGGCTC
TGTGATGGGGGCCCTAGCATTGCTGGAGGAACATCCTGGGTAGCTAAAGTACAGAAACATGGGAGTGAGGAGGTCCTAAGAAACCCTCCAGTGT
GCAGATTGCTCTGGCCCCAAAGCACTTCTATAAACACTAGTCAGAGGCCAGGTGTGGTGGCTCACGACTGTAATCCTAACACTTTGATAAGTGG
ATAAATGTGGGAGGCTGAGGCGGGCAGATTACCTGAGCTCAGGAGTTTGAGACCAGCCTGGGCAACATGGTGAAACCCCATCTCTACAAAAAAT
GCAAAAATTAGTTGCATGTGGTGGCATGCACTTGTAGTCCCAACTACTTGGGGGGCTGAAGCAGGAGGACCTCTTGAACCCAGGAGGTCAAGCC
TGCAGTAAGCTGAGATCACGCCACTGCACTCCAGCCTGGGTGACAAAGTGAGACCTTGTCTCAAAAACAAAACAAAACAAAAAAATCCACTAGT
TAGGACAGGTATTATTGCCCCATTTTTACTGATGTGGTAGCTGAGGGCTGAGATTTCCCTCAGGGCCTACAGCTAGTAAATGGCAGACCACATT
CTTATAAGTGATTCTGCCCCAGCATTTAAACCCTTCTTGCCACCTCCAAGCCTGTGTGACTATAAATGTTAATTGGTAGAGGAGAGGTACAAGC
AGGGTCACTGTGGAAAGTTCAAGGCCAGGAGCTAGGGTCAAACTCCCACTGTTCCTCCACCCACAGTGGCCTTTGTGACAAGAAGGGGGAGGGG
TGGGGACTGTGAGATCTGGAGGCTACCGAAGGTTACAGAGCCTGGGCTGTCCTTTTTCAACGGTTGAGGCACAACCCATCCCTGAGTCACTCCA
GTGGGTTGAGGCGTCCCCAGGCATGGAACCCCCTAGAACCCAAACAAAAGTGGTCCCTAATCACTGGATCAGGCATCCCAAGGAGAGGGCCCTC
CAAGGATGGGGCTGGGGAAGTGTGTGCCACCGCAGACTACCCAGGCTCCACATATTTTCCACCTTGACCCCCAGGCCCTCCCTGAAAGGGCAGA
TTTGTGCTGAGCTTTAAGTGGTTTCTGTGGTTTAGGGTTGGGGAAGGAAGGAAAGAGATGCTGGGGACTGCTCCCCAGTTCCAAGCCACCACCA
CCACCTCCCATCCCTTCCAGAGTGTTTGCCCCCAGCATGGAGATTTCCCCAATAAGGGGACTGGGCACGTGGGGATGAAATGATTCTCTGATCC
CTATCAGCCCCTTCCTTATATAAGATTGTATTTGAAGAGGAAATAACCACCGCCCTGCTTTGAAAGGACGATTCTTCCAGATAAGGGGCTGTGA
TTGCAGCAAGAGGGAGAAAGTTTAGACAAGAGGGGGAACTTCCCAAAAGGATAACCAAGGGATCTTCTTGCCCTGGGGAGAAAAAAAACTCAAA
CTATCCATGCTTAGGGCAGGGGAGAGGTAGAGAATGCCTAGGAAGCATGTGACACTCCTAGGCCTAGAGGTCAGGATACTGTCTTCTAAATCTC
CAGTCACTTCCTCCCCACCCTACATAGTAGCACAAAGTGGCACAACTGGGGAGTCAGCTGTGCCTGCAGTGTCTGGGGATGCTCTGTGTCCATC
CGCAAGCATTCTACGTGACCTCCACTCTGTGCCTGCAATTGGTGGGTTGTAGAAGAGGTAGTCACGAAGGGCCCCTGGGAGACCCAGAGTCAAG
AGGAAGAGAGGCCTAAAAAGGAGAAAAGATAGAGAGGGGACAGCTGCAGAAGAGCCACAGACAGGCAGGAGCCAGCAAAGAGAGACAGAGAGAT
GGGGGATAACTGAGGAGTTCTGGGGAGGAGGGGAGCAGAACGGCATCTCCCCTGCACTCCCACGCCCGGCATGTTGCTGGCTCCTCCTGTCAGC
TGGAGGGTGCTGGGGGGGCGGGGGGGAAGGGAGCTGCAAAGCACTTTACCTCTCACTCCAAACCTGCCCAAGGTGCAGAGCCCCCAAACAGACC
AACATCCTGTTCTGTGCAGAGATGCATACATGGATGCGTGGGCCCACATTTACCAAAAGAACACGGGGGATAATCAGAGCCTCAGAGGCATAGG
CACATACCCTTAGACTCAGTGCAGAAGGTCCCCAAAACTTCCCCTCCTCTTTCTCACTACTCATCCACTGTCAATGACCCTAGGAGTTGAAAAG
TCCTTCTCCACTTCTACCTTTATCTCTCAATCTGCAGCCATGGGGAGACTCTGGGAGCTACCTAAGAACAGAGGTGCTGATGGTAGCAGAGTGT
GAAGCTCCAGGGTTGGGCAGGAGTGGAGGATGGCGAGACGAGACGGCAGAAGAGGGAAGGGACAGCTGCTTGCCCTCTCCATGCCAGGTGCCAC
GTGCACCAGGTGGGGGGGAGTGGGCGAGTCACGAGGCTGTGGCATGGCTGACTCCTGCTCTGCTGTTTACCAGCTGGGAACGAGAGCAGGAGGA
GGTGCCAGGGAGCCTCTTGCTGCAGCCTGGCCCCTGGCCAGTTGCTTGTTGGGTAACCAGGAGAGTCAGGCCCAGCTAATCAAACTCTGGGCCC
AGGCCATGCAGGAGAAAAGCAGCACCAGGATCCCCAGAAGCCTCGTCCTCTTGGCCCCCTTCAGAGCCTTCACTGCAGGCCTCAGTGTGGGCCA
CTAGGTCTGGGAGCCTCTCCTTCTCCCAATGAGTCCAGTGCAGCATCTCTCCTCTCCTCTTCCCCACCATGGCTTCTCTGCCTCCAGGCACTCC
TCCAGGCCTCCCCCTTGCCCCAACAGATGAGGAGCACGTAGAAGCATCAGGGGGTCAGGGAGTCCTGGCAAGAGCAGCAAGGGTTAGGGGCAGG
GGCCGAGCAGGCAGAGGCCTCAGGGAACTGAACAGTCACTGGGAGGAGTCTGACTGCATCCTAGAGAAGAGACACAGTCCCCCAAACCTGCCCT
CTCCATCTCCCCTATGCCTGTCACCTGTCTTAGGTAGAAATCACAGGATTCTAAAGTCACAAAGACTTGGGTCCAAATCATAGCTCACCCATTT
ATTGTTCTTGTGTGAGTCCTTGGGCAAAATCATTAATATCTCTGAGAATCCCTCCACTCACTGATCAATCTGGAATAATAATGTTTCCCTGTGG
TGGGTGCTGTGAATATTTACACAAAGTAAAAGCATGGATTTACATAAAGTGTCCTGCAAAGGGCCCAGCACACAGTAAGTGATTAATAAATGCT
AGTTCCCTTCCTTTCTCCTGTCCTGTTTCTCCTCAAGGCTCCATGCACTGGTCCACTGGTCTCTCCTCATGTCCCCTCCTCGTGCCAGGACACT
CTGCCAGCCACTCCTTTTCCCTGCCTGCTGGAGGGCCAGGTGCTCCCGCCTTCCACCCTCCGCCCTCCTCCCTCCCCTGGGCCCTGCTCCCTGC
CCTCCTGGGCAGCCAGGGCAGCCAGGACGGCACCAAGGGAGCTGCCCCATGGACAGGGCCCCACAGAGACAGCACCGAGCCTCACGGGGTAAGA
GGCCCATGGCCCTGCCTGCAGGTGGAAGAGACTGCTGGAGGTTCAGAGGAGGGCAGAAATGGCAAGAGACTGGGCAAGAAGTGAGGAGGGGAGG
GAGGGACAAGAGTGCAGAGGAGAAAGCCCTGGGTTGGCAGGGAGTGAAACTGGAGGAGAAACAGGAGGAGGGGCTGGGGAGATGACAGAGGAGA
AGAAATCGCAAAAGGAGAGAAACAGGAAAGAAAGGGAGAAGCGGGAGAAGGAAGAAACAGGTTGGAGGGTAGATGGGGAGGGAAGAGCCAAGTC
AGGGGAGCCAGATCTAGACCCAGCGAGGGGTCAGAATGTGGACAGGCCCTCCCAGGTGTGGGCTTACCCCCCAGGAGGCACAGGGACACTGCTT
CCTTGATCTGGCCAGGACCCCAGACAGCAGTGGCCGGGAGGCTGGCTGCTCCACGTTCTGTTCATTCACCAGTGGAGAAACCTGTCCTTTAAGG
AGTGACAGACTTGGGGAGTGTGGCAGGGGGAACCACACCCTGTCAGCTGCCCATGACCACGTAGCCTGGTCCTTTCCTCGCCACTCACCCAGGG
ATGTGGGATTTCATCTCTGCAGAGAATGTGGGGCACTGGGCCATCAGCAGGGAAGGACAGAATGTCAGGCCGTCCTTGGCCCCACCCATGGTCC
AAGTCAGGAACCCCCTCAGAGCAGGATCGCAGGGCCACAGGCTTGGTCAGCTGTGAGCATGGAGGAGGAAAGTTTGAGGGCCAGAGCCACCAAG
CTCCAGGGCCACCTGGTGGAGGAAGAACTGGCTGCCTGGGTGAGCAAGGGGAGCACCCCCACCACAGGACTGCTCTGAGGTGCAAGGCTAGAGG
CCAGGAGCCGGTGCCACGTCAGGGAGGAAGCTGGGTGGGGCCAGGCAGGAGCAAGGGGAGGTGGGCGGGGCTGGGTGGGGCCTTGCCACCTCGG
GGCAGTCTTTTCTCCTCCCTCACCCCAGCAGAGCCCAGGCCATCAGGCCACTCGCCTCCACACAGCCTAGTGACCACGGCCTGCCAGTGGGGAG
CATCTCCCGGGCAGAAGGGCACTGTATGTGTGTGTGTGTGTGTGTGTGTGTGTGTGTGTGTGTGTGTGTGTGTGTGTGTATGTCAGGTGTCAAG
ACTGAACAGCAGAAGGAAAAAAATAACAGAATAGAGTCAGAGGTTAGGGAAGCTATAGGGGGTATAGGGCATGGTTGAAGCAGTCTTTTCTCCT
CCCTCACCCCAGCAGAGCCCAGGCCATCAGGCCACTCGCCTCCACACAGCTTAGTGACCACGGCCTGCCAGTGGGGAGCATCTCCCGGGCAGAA
GGGCACTGTGTGTGTGTGTGTGTGTGTGTGTGTGTGTATGTCAGGTGTCAAGACTGAACAGCAGAAGGAAAAAAATAACAGAATAGAGTCAGAG
GTTAGGGAAGCTATAGGGGGTATAGGGCATGGTTGAAGGGCTTTCTGAGACAAAAGACTGGCCATGTGTTCTGTGATGAGCTGCTATGTGGATG
TAGGAAATGGGCCAAGGTCTGGCAGGGGAATCCTCTTTCCAGGGGCTCCCTGGCTAGGTGTTTCATAGATGTTAACTGCATGAATGGGTGACAC
CCCCCCACCCACCAACCATGCTCCAGCTAAGGCCCTCCCCTCAACGGCTGCTCCCCCTGGAGGTCCGAGTATCCATGTGACCCCCATCGACCCA
CTCATGCTCCCTACTGCCCAATCTCCACCCAGACCCGGCCACGCAGCACCAGCAGAGAAGGGAGGCCTTCCTCTTCAGGCTGGTAGGCCAGGGA
AGCCTGGCCCGTGGAAGCCAGAGAGGGGTCTGTATTTGCAACTCTCCCCTCCTCTTCCTCCAAACATCCTCTCCCCCTACTCCCAACCCCCACA
TCTCTGAAGCCCTGGTGTTTTCAGGCATAGATAAGCAACCAAGAGATTTCAGTCCCACAGAGATTTAACTCTGGCTGTAGGATATTTCTTGTGC
ACCTCCAATGAAATTACAAAGTAAATAAGACCAACACAGTCCCTGCCTTCACAGGGCTTTACAATTTGGCAGGAAAGACCCATTTCATAGACAA
TGACACAATTACAGCAGTTAGGTGACAGTTGGTGACATGGTGAGTGCCAGGAAATAAAATGCAAGGAGCTAAATGTGTCAGCACAAGGGCAGGG
GAAGGGGGTGTGTGTGGCTTCTCCAAGGAAGAAACATAATCAGAGACCTGAGGGAGAAAGAGAAGTTGGCTAGATTAAGGGGTACAGGAGGGAC
AGGCATTCTAGGCAAAGGAGCAGCTTGGGGGAGTCCCAGAGGCAGTGAGGAGCCTGACACTTCTGGGTACCTGGAAGAGGGCCAGTGTGGCCCA
AGGGCAGAGGAGGAGAGAGGGGCACTGCAGGGGGTGGCCTGCCTGCCCTTCAGACGCCAGGCTCTGAGGGAAGGAGTGCGGCAGTGGGATCCTA
GGAGTGGGGAAAGTGTCTCAGCCCCTCATGATCTTCAACCTTTGACGTCAACCCCCTCTCTTGTCTTTACAGAGCTGCTGGCTGCAAAGAAGAC
CCACACCTGTGAGTAGGGGGTCCGGGGCAAGAAAATGGCTGGGGCATAATGAGATGGAGAATAATTCAGCAAGTGATCTGTGGGGGGACTGAGG
TTGGACAGAGGTGGAGGAGTCAGGAAGAGATGGCAGGCAACAGGAAAGCAAGGGGAGGTGCGAGGAAACCACAGATCAGGGCAGTGGGAGGAGA
TCAGGAAGAAGCCTGAAGCATGAAAGGAAACACTGTAAAGAAACTAGGAGAGAACAAAGGAAAAGGGAACCGCCTTGTGAAAAAAGGCGAGGAG
AAATGCTGGACAGAGGCAGGGAGATGGGAGGGGGTGGGGCCGACTGCCAGGCCGGGGAAGGAAGGAGATAGAGGTAGGGGGGTGTGCGGAGGCG
GGGTGGCCTCAGGAACTCAGGAACTCAGCGCTGCTGCCAAGGGAGACAGGGAGCCTGCACCAGGCTTTTTTTTTTTTTTGAGACAGAGTCTCAC
ACTGTCGCCTGGGCTGGAGTACGATGGCACAATCTCGGCTCACTGCAACCTCCACCTCCCAGGTTCAAGCGATTCTCCTGCCTCAGCCTCTCGA
GTAGCTGGGACTACAGGCACCCACCACCACGCCTGGCTAATTTTTGTATTTTTAGTAGAGACGGGGTTTCACCATGTTGGTCAGGCTGGTCTCA
GACTCTTGACCTTGTGATCCACCCCTCTCGGCCTCCCAAAGTGCTGAGATTACAGGCATGGGCCACTGTGCCCAGCCTGCACCAAGCTCTGTGG
TCCTGAGCCAGGTTTGGTGTTCATCTCTGTGGTTAACACATCCTGAGGTGTGAACAAGCCTGGAGCCAGCCTGTGCAGGACTAGAGAGCAGGAG
AGCTTTATCTCTGTAGGAAACAGAATGGACAGGGGCACTCTGTGTCCTGGGGCCCTGAAAGCCAGACTACAGCAGGTAAGAAGGGCACTAGAGC
TCACAGAGGACTTCCCAGTGGACGCTAGTGAATAAGAATCCTCTCTAGGGACCACTCAGCACATCAGCTCTCCTCTCCTAGTTTGTGGCAAAGG
CTAGTCCCCCAGAGAATGCACACAGTCCCAAAGCTATGCCCATCAGGGCCATGCGTGCCCTTCTGTGAGCATGGGTCCCTGAATCGAGAGGATC
CCTCCAGGGTAGGAGTGGCCACACCATTTCTCCATAGCCGTCCCTTGACTGCCTTGCCAGTTCCTCCTGAGAGCTCACTGCCATCTCGCCCAAT
GCCAAAATTGTCTAGAGCCTCCCTGTTCCAAGTAGAGACATCTCAGAGAGCAAAGCATTGCCCTTCTCCATGAGCTGGGCAGCCCAGGGCTGAG
GGCCATCCAGGATGCTACCTCCTCCTGGGTGGGGGTTGCTTTGCTCAGCATAATCCTGATCAGTGTCTCGCCTGCCCCTAGCCACAGGGCCCCA
CGGAAATGCTGAGCAGGGCCCCGGGAGGAAAGCGGGTCAGGGAGTGACGCGGGTGAGCTGGGGGAGGGGGGTCACGGACCTTATGTGGGTGGGG
GGACTAGGGGGACTTTCGGAGCAGCAGGAACTGGGAGAGGTCAAACCCTATTCCTACCCTTAACCCCTGACCTCAGAAACCTCAGCCAGGACTA
TACCTCCTTATCCCTTTCCCATCCCACCAGTCCTCGTGTCCCCACCCCAAGAGTGTTGTCCTGAGGATCCAGGCTGACAGCCTCTCCCCCACTC
CACGGCCCAGCTCCTGAAGCCCCAGGAAGGCCCAGGAAGGCAAGGAAAGGCCTGTGCCTGGATGAGAGGGTCTGGAGTCCCAGCAAGATCAGAC
ACCTCTCAGGAGTTTGCCCCACAGAATCCAGGAGACATTAAGGCCTCCCCCAGGCCGAGAGACTCTGAAAATTGGCTCCTGAATTGAGACAACC
CCAAGATGGGGGTCCAAGATGGGGAACCCGGGGGAACTCCCCAGACCAAGAGGGAGCCTGAATACTGAGGGGCCCTGAAGAGGGACTCTCAAAG
CCAATGTACCCCACAAAGCACCTAAGAGGCTGAGAAGGTCTCTGGAATTGTTTGGCCCCTGAGAAGGACCCCCCTCCCCCAGGCCAAAAAACAC
TCCAGAGAGACTCACATACCGAGGGACCTCCCAGGCTGAGACACCCTCAAAAGCAGACTTGAACCAGAGAAACCCTCCCATCCTTCAAGTGGGA
TCCGAAGCCAGTCTGCCTGGGCTTGAATCCTGACTCTGCCACTTACTAGCTGTGTAACCTTGGGCAAGTTACTTAACCTCTCTGTGTCTCACTT
TCCTCCTCGAAATGAGGATAATGGTAGCTACTTCATAAAGTTTTTTTATTTTTTCTTTTTGAGACAGAGTCTTACTCAGTTGCTCAGGCTGTAG
TGCAGTGGCATTATCTCAGCTCACTGCAAACTCCGCCTCCCGGGTTCAAGCGATTCTCCTGCCTCAGCCCCCTGAGTAGCTGGGACTACAGGTG
CTCGCCACCATGCCTGGCTAATTTTTGTATTTTTAGTAGAGATGGGGTTTCACCATGTTGGCCAGGCCGGTCTCGAACTCCTAACCTCAGGTGA
TCCACCCACCTTGGGCTCCCAAAGTGCTGGGATTACAGGCGTGAGCCACTGCCCCCGGCCCACATAAAGTATTTTGTGAGGATTAGATTATCCA
CATAAAACATCCAGAACCATTTCTGACACATAATAGTAAGCACTATTATTATGATTATGAGTATGATGATGGTGATGATGATCATTATCTACAC
TCCAATTTCAGCAGTTTGGCTCCTAAGGAAATTTCTGGTTTCCTTCTGTGGATTGTGGGTATTTGCCTGGTGATTATTACTGCTTCTATCATTT
CCATGTATTCCCTAGCGCAAGATATATGTGGTGTCTGCAGGGAGGAGCGTGGGCATGGGAGTGGTGGGACAGCCCCCCAGGCTGCACCACACTG
GGTATGGCTGTTGGCAGTCCACAGGGTGTCAGGTGGCACATGCCACCACCCCAGGCAGACATCCTGTGACTTACCTCTGAAAACATCCCTGTCC
CTGAAGTCACCTGGCAAAGAGGGTCAGGCTTGGAAGGTCCTCGGGGGTAGGAGGAGTAGAAATTTGGCTTCAGGCTCGTGTGAGCTCCATTCTG
GCTCCCCAGCCAGAACTACCCTGGCGGGTGGAAACAGCTTTTACCGCGTGTGGCTGTCGCATGTGGTTTTGGAATTTTCCAACGCCCCCTACGA
TTGGCTGCCCCTCCCCTCACACCCTGCCCCAGGCCCAGATTGGCCACGTGGGGCGCCTGTCATCCTACTCACTGCACCCCTTGGGGGTGGGGTG
GGGGGTTGTCACTTGGCCACCTGTGTGGTGCACAGCTTAAACCCCCCAGTCCAGAAGCACTGGGGGAGAGAGCTAGGTGCAGAGCTTCAGGCTG
AGGCGCTGCTGAGAGGGCCTCGCCCCGCCTCTGCCGCCAGCTGCACCCCACTCCTGGACCACCCCCCTGCTGAGAAGGACAGGGAGCCAAGGCC
GGCAGAGCCAAGGCTCAGTCATGAGAAGTAAGTGAATGGGGCCACCTGGGGGCGGGGGAGCCTGGACCCTGTCGTCACCCCTCTGGAAAGGAGG
ATGTCTTGGGAACAGTCTGAGCAAATCTGAGCAAAGCAGGGTGCGTCCTGGTCAAGGTCAAGATCTGTTGGGAGATGGGTGGAGCAGCTGGGGA
GTCCTGCTGGCCAGGCGGCTCCCTGACAGCAGCAATGGTAAGAGGACTGTTGAGGAATTCCTGAGTTCCAAAAAGAACCTCCAAAAGTATCTGA
GGTATCCAGGCCCTTAATTTTACAAAAAGACTGAGAGCCAAGGAGGCCGCGTACTTGGCCCAATGTTACACAGCTGGTATCTGATGGGGGCTGG
TAATTACTAGGGGCCCTGAACGCACTCTCCTGTTACAGTGTTTCCCTTGTTACAGTGTTTCCCTCTGTGCCTCCAGCCTGCTTGTGAGTAAAAG
GAGGCCAGAGCAGTTGCTGTGCTCTGAAGCAACAGGACAGCGAGAGTCAACTGAGGTTGGCAGGCAGCAGGGCACAGAATCCCCGCGTCCAGGC
CTCAAAGCCACCAGGCTTTCTGCCCAATCCAGGGACATGAGGACTATGTGGAATCCAGTCAGGGTGCACCCGGGCACATGCCTGCGTGTGTGTG
CATGTGTGTGTGTGCGTGTGTGTGTGGGGTGTGTGTGTATGTGTGTGTGTGGTTCTCCAGGGTCAGTCACCCATTTGACCGGGGAAGCAAGGCA
ATGGAGATGGGATTGGAAAGACCTCGGCCTCACCCTTTTCCTGGCTTGGAGCCCTGGGCCAACAGTTGGTGGGCAGATGTCTGGGGTTCCTGTG
CCTTCCAGCAGAGCAAGGGTGGGCAACCTCAGGAAGTCCCCTGTATTTGTTGTGAAGGGAAGCCAGGGAAGCCAGAGAAGCTCTCAGAGCACAA
GGTGAAGGAGGAAAAGGGAAGCCCCATGCCCCTGTGGCTGAGGCCTGAAGCAGCCCCAGCTTCAACCTGGGGTCTTCCCACAAGCCACAGAAGG
GAGGCCGTATTGAGTCCTGGAAAGACCCCTGGACTGACAGTGTAAGACCAGGCACTTGTGCCGCCTATCACTGGCTAGCCATATAGACTTAGAC
AAGCCAAGTCACTCGGCCCTGGTCTACCATCCGCAAAACAAGGGAGCTGAATTTTAAGATAGAACAACAAAACCAACTATGGAGAGTCTCCCAT
TCCAAAGCCCCAGGGCCAAGAGAGAGCCCAGTGCCCTAAGGCAGGCATCCCCAAGGGTGATGATGGAGACTCAGCTCTCCCAGAGGTTAGAAGG
AAGGGAAACCAAGGAAAGAAAAGAATTCCACTTCTGTGACAGGGACTGAGGCCACAGTGCAGGCATATGAGCTGGGGTGGATAAGGAGGGCTGG
AGAGAAGAGCCAACAGCTTCTACACAGAGGAAAGGGCCCCTTGGAGCTTCCGTTTCCCTTCTAGCTTCTCTCTATTAGCAGCAAACGCTGGCAG
ACTGGTCTTCCCAGAGATGGCCCCTAATGAGCAAATCAACCCTGGGATGAAATGACTAGGTGTGCTTCTAGCCCAGCAGCGGGGCAGCAGTCCC
CAGGAGGGGCTGAGGGAGACACTTGGCCAGCTCTCCATTATTCCCACCCCCACCACACACACACAGCCCCACCCAGGCGGGGAAGTAGGAGAGA
GGGCCTCCTCCAACGAGGGGGTCAGTGCCACTGGTCCTTCTCACTTGGACATTTACAGGTATCTCATAGCCCCAAGTTATCTGACCCCAATGAG
TGCCAACCACCACCAGCCCCTCCAACCGCCGATGGCAAAAGTCAAAGCACATGGGGCTTGGGACTGTGACACACCCAGAGACCAAGGGCCCGAC
AAAAGGAGCAGAAACAAATCAAGAAATAAGGAGAGATGCAGAGGCCCAACTCAGAGGGCGAGTTTCTGGAACACCTAGGGAGAGAGTAATGTCC
AGCTGGAGGTGTGGTTGGAGAAACACCAATAGAGCCAAACGCATGGAGGTGCAGCCGGGCGTGGTGGCTCATGCCTATAATCCCACTTCAGGAG
GCTGAGGCTTGAGGCCAGGAGTTTGAGACCAGCCTGGGCAACAAAGTGAGCCGCTGTCCCTACAAAAAAAGTTTTTTTTAATTCGCTGGGCATG
GTGGCGTGTACCTGTAGTCCCAGCTACTCAGGAGACTGAGGCAGGAGGATCGTTTGAGCCCAGGAGTTCAAGGCTGCAGTGAGCTATGATCAAT
GTCACAGCACCCTAGCTTAGGTGACAGAGCGAGACCCTGTCTCTTAAACAAACAAACAAACATGAAGGTGTGAAGCCCAGAAACAGAGCTGGAA
TGCAGAAGATCACAAGGCACATAGACACAGGGAGAGGCGGAGAGAAAAGATAACTGGCAGTAAAGGGAAGACAGGGAGACACAGGGTCTAGGCA
AGCGGTAAGAGGGAAGTAGGGTGGCTCTGACAAGGATGGCACCAGCACCCTGCCATCCGACCTGCTGCTGTCAAGTGGAGATAATGTGTCTGTA
CGCCAAAGAGACTCCCAGCCAGCCAGACAGAGGGTAGCCCAGACATCTTCCCCTAAGGGCATGGCCAGCGTGGCGCCAATAGGCACTCAGGCCC
TGGCATTTGGTCTGGATGGGGAGGTGGGAGGCCGAGGGGTGTGAGGTCACTGTCTATGAGCTGATCTTTCTTAGGTTTGGTCATGATCATGAGG
AACAGTGGGCCAGAAAGGACTCCAAGTCCCCAGGGTACACAGATATCACCCTGAGAGCCCAGGGCCATCTTAGGACTCCGTGAAGGGCTTCTTC
CTTCCTTTCATCACTCCAGCACCCTTCTCTTTCCCCGTCTTCCCATTCCTAACAGAAATACCAAACCACCGAGCCCAAGGTGCTAGACTCTGAA
AAGACTCAGCTTGTCCCAGCAGAATGAAAATATTCGGTCCCTACCAAGGGCTCCTCCCCATCCCCTGCCGCTGTGCTGCTGACTCAAACTAACA
GGGAACAGGACTTTCCAAGGGCTAAGGTCTCTCCCAGGCGGGCAGCCCAAGGCCCGCGCTCCTGTTCCCATATAGCTAGTCCTTTCTGAAGTCT
CACCTCCACGCCTGCTGCTGCAGTGCAGCCCCTTCCCTGGGGCTCTGAGGCTCAAAAAGGATGGAGATGTCTATTTCGGGGGAGGGACAGTAAC
AAGGCAGGGCCTCAGGGCCAGAGCCAAGGTCTTTCTGGGTCCACTACCCTCCTGGAGCAGTGAAGGTGCTCTGGGATGAGGGACAGATGGCCCT
GAAGGCAGGGAAGGTGCTGATGGTGACGTCTGGGCTCCCACTCGCCAGAGCTTCCTCCTAGTGATTCATCCCCTCCCCCATTCACTGGTTGTTT
TCACTCGCCTTTCTCCAGTCCCAGACTGTGGGGGTGGCGGAGGCACCAGGAGGGGGTTTCGGGTGGCTGGCTGGCTGTCATTCATGGCTTTTCA
AAACCCCAGACTCTCCCTCGCCCACCTGAGTTTTAGCTTCACTATTTTCTCAGCCCCAGGATCTGGGTGTTTCAGCAGAAAATTCTCACCAGGA
GCTGCGGTGAGGGCCCTGGCCTGGGCTGGGGGTAGTGTTCGCCAGGTAGAACCACTTCTCCCCCAGTCCCCACCCAGCCCTCCCGCTCCTGCTC
CCCTGTGGTGGCACCCACCACCTTGGTTTCCAGCTTTGCAGAAGCTGGGGGAACTGAGAGCTGAGAAAGGAGGGAGGCTGGTGACAGGTTGGGG
TGTGCAAGTGACCCCTGACCGGAGCTGTGGTCTCGGTGGGGATCGAGACAGAGAGTTCTGGCTGTGGGGAGAGGAGGAGCTCAAGCTAAGGAAA
GAGATGCATTCCTCTTTCTCACTCATTCATTCATTCTGCAAAGGGAGGCATACCAAGATGTATGGAAACCTAGTCTATACCCTAAAGGGGCTGT
GGTGTTCCTGAGGAGTCCCCGGGAACCCTGTTGTTGATATATAAGTCATCCCACTTGGCCTTAGAGAAAGGGTTAAGGGCAGGAGGAGGCTGGG
ACCTCAGAGGAAGAGTTGGGACAGAGGGAATAATATCTCTTGGGATCTTTATACAAACAGGAGTCCTGGCTTTAGTTCTGTGGGTTTCCTCTGT
GAATTCTGTTCCTAGACCACCACAATCACCTCACACTTTTACCCCTACAGCCAAATAACCATGTCCTCTACCCAGGTCTCCGGTGTGCATTTGG
GCGTCTTATCCTCATAAAGTATATAGCTTAAGGCTTGAGGGCACTGGCTGCAGAATTACAGAGATCTAGGTTCAAATGCCAGATCTGCCACTTC
TGGCTGTGTGACCTTAGTCAAGCTGTTTATTCTCTCTAGGCCTCAGTTTTCTCATCTGTAAGATGAGGATGATACTAGCAGGGCCTATTTCACA
GGGTGGTTGTGATCATTGAATGAAAATGCATGTAAAGCCTGGCACAGAGTAAGAATTCAGCCAATGCTTATGTCTATCATTATCCTTTCTCCTT
AATCAGTATCCACTGGAGAAAAAGGAGAGAGAAAGAGGAGAGGAAAAGGCCCAGCTCTAGATTGACGAGGCACCAGGAGGAGATGAGGACCCCG
GGGTTTCTCTGTGTGAACCCATTCTCTACCATGATGGTGGGGGGGTGGTGGTTCTGGGGTTTGAGAGGAGAGATCCCCCTGGGTTGCAGCCCCT
CCCCTGCTCCTACCACTTCCTCTCTGTGGCATAGGGCACCTGGACAGGGCCCAGGGTTGGCTTCCTAAGCTGGCACAGGGCCAGGCTCTGGAAG
TACTCAAAGCACTATCATTGAATCTCACAGCTGTGAGAGGCTCGATTAGAGGAGGATGGAGGACTTTGCCTCTACCGTATTTCCCCTCATAGGA
GGAGCTACTAGGATGCTCCTGGGGGTACTTCAGTCTCTAAAGCAATTCAGAGGAGATTCAGTTTCTTTTTCTAGTATTGTCTCAGAATGAAGTC
TTAGAGGTCACACTGGATGGCAGAAAAGATGCAAGAATGAGCTCAGGAAAAGATCGCCAACCCCAGCCCCACTCCCTGGAAGGAGCAGCCTGGC
AAAAAGGCAGGAATAGGGACTAAGTTCTTGCTACTCAAAGTATAAGCTGAAGACCAGTAAGCTTAGGCACCAACTAAGAGCTGACCAAAGTGCA
GAATCTTGAGTCCCACCTCAGATCCCCAGGTAATCTTGTTTGAGATGCAGAAAGCCATAGTCATTCAGTCATTAGATGACTTAAGGAGCTGGCG
GGTGCATTTGGGCATTTGTCTCTCCTTTTGGAAGATGTGGAATTGGAGTTTTCTTTTGGAGAAAGCTCTCTCTCAGGAGAGAGCTACAGTGAGA
GAGCTGCAGGTGAGATGCAAAGATCTTTCCAAGACAGCAGGCGAGGGTAGAGTTAGGGCTGCCATGGGAGCAGTGGTGCAGGGGCTGGTTCGAG
GTTCCTGACCTTTGCTTGGCTCTAGTTCCTGGCACTGGCAGGCCCTTAGATGGTAAGTTCCTGGCAGGCCTGGCTCCAGGGCAACAGTTTGAGT
AATCCTGTGATTACTCAGCTCCTTCTCCCCAATCTGAGTTCCCACCACCTGCTGCTTCAGGGACGTGTCTCCCAGGGGTAGCTTTTGAAGGTGA
GTTATCATCAGAGGTGTTTTCAGCTCCTCTCACCAACCCCCAAGTCTACCAGCCCCCTTCCCGTTCTCCCCCACCCCTTCTCTACAAGTAACAC
ACACCCTCATGTACACAGCACTGGTACTTTCTTTCTGAGGTTTGGGTGGATGGCATTGAAGGGAGTGCAGGTGGCAGGGGAAGGAGGGGGGAGC
AGGACTAGAGGGCCTGAAGTGCTGAGGGGGCCATGGGTTGGGCAAAGGCCAGGATACAAGCGTGGATTTTCTGTGTTTCAGTGTTTCCGCAGCC
GTCTCTCCATCCCTAGATCTATGTCTCTGTTTTTCCTGGACCCAAACCTCTTGGTCTCTCCAGCTCAGTGAGGCTAGCGGGTCTGACATTCTAG
CTCAGAGTCTTTGGCCTTGTGTGTCTGTCTCACTGGGTACTTCTGTCTTGGACTGGGAGGATGGAGGATGGGCTGACCAGGTGGCTGGAGTCCA
AGGCTTTGGTGTTGCCCAAACATGGGTATTTGCGAAGGGGGTCCTTGACTGGCCGCATCTTCCTATGAGAAGGTCAAGGTCTTGGTGACACTGT
TGTGAGGTTCCTAGGGCCAGGAGAAGCAGTGGGTCAAGCATGGGGCAGGGAGATGTGTCTGGGGTCACCATGTGGTCAGCACACACACGCACGC
GGATGTGCCTCTTGGAGAAGGGAAGAGGCAGGGAGGCCTGTGGTTGACTGCTGGGCCAGAGCCCGAGTTCCCACTGCGGCTTGGCCTAATCTCC
CTCCCGCTTACTTTTTTGGGTGGCCCACGTTGGTTTCTCCCATCTCTGGTCTTAGGCCACTGAAAAGAGAGATGCCACACTGTTGGAGGGGGAG
GTGTTGGGCAAGAACAGAGTCCACAGGGCCCGAGTGCTCCTCTCAGCCTTGCCAGGCCCACTGGTCTTCTCAGCCTGACCTCTCCTCAGGAGTA
ACTTGAATACACAGTGCTTTCCATGGGTGATATCATTTCATTACTCTCTGTTACCTACAATCACCCCATGGACAGGCAATAATACTTTTATCCT
GATCTTGCTGACAAGAAAACGTAGGTCTAGAGGGAGAATCTAAGTGACTAGAAGTATGGGGGCCCCAACGTTTTCTGCCCCTGTTCCAGACACC
CAGTGCCCTCTTATTCTCTCTTCCCCTCCATGTATTAAAATCTGCTCTTAATTTACTGGAAAGAACCTGGAAAACTCACTTACCGCCTGGTCCT
GGGGGGACCACAGCTTACAGTGTGAGCACTGGAAGCTTGCTGCTGGGCAGGCACGTGTGGCTGGGGGTTGGGTGAGGAATTGAGTGCTAATGAA
GGGATTTCAGATACCCATGTGCCCTCTCCCCGTCAGGGTCAGCTACTGCCTCTGACTCACCTCAAGTCTGGCCTGAGGGATGATGGTTTAAGAA
TGATGGAAATAATATTTTGGGGAATGGCGGGCAAGCCATGGTCTCCCTGCCCACTTCACATCAACAGGCCCTACATGTGACCAGTGTCCAGGCC
AGGGACTGGAGGTGAGATCACAGCAATGTGCATTCGCCCTCCACAGAGGCTGCCACTTCTTTTGCCCCTTTGGTGGGAGACACGTCATAGATTA
AAAGGGGCTTTGGCCCTTCTCTTCACTTTTCTGGCCTGTCCCGCCCCAGGGCTCCCCACTCAGAGGGGAGGCAGCAGGTCAGTGATGGTGGAAA
AGAGACCCATGTGCTCTGGGAAATGCTAAAAACACCTACAGTAACCAGGGATCCCAAGCTTTGCCCAGCTACCTTTCAAGGAAGAGCTGGGAGA
AGGCCTCCTGTGGTCCCATGAAGCTCAGCAGAGAGTCGTGGGCATTGACAGGAACAGGGCAAAGCAGAGATGAGCCTAGGGCCAGCAGCAAACC
ACAGGGGTAGGGAAGCAGGTACAGGTTACAGTGAGTTCAGTGGGAGAGAGAACCTGGGGTCCTAGTGCCCCATGAGCAGACACTAACATACAAA
TATGCACGTGTGGACGCAGGAGCAACCAGATATGTCGTAGGCTCACAAATGACACATACTTTTCTTGCATATGCACACCCACAAGCACACCTTC
AAACATGCCCACTGAAATGTGTACCCGAAAAGACAGGTACACAGAACAGCATAATGGAAAGCTCAGGGGAACTGGGATGTAGATCCAGCTCTCA
GGCAAACTAGCCTATGACCCTGGCCCAACCGCTTTGCCTCATCTGGCTTAGTCCTTCATCGGTAAATGAAGAGGTTGAATGAGATGGTCTCTAG
TCTCAGCCATTTTTAACATTGATGATTCATGTTCACACAAATGGATACAGATGCCTTTGTACAAACATAAGTGTTCACACTCAGACACACGGAT
GTACACAAAGCCACCCTCTCTCTGCTCTGGGGCCAAGAGCCTAAGAGCCCTGGCTAATTCTTTCCCTAGGCTCTCAGGCATCCAGCAGAGCTGG
GGTGTTGAGGCCCCGTTTCCTGGGTTCCTCCCTGCCATCCCCTCACCCTGTCTCTGTATAGCACCTCCCTGAGCCTTCACTGTCTGGCTGGGAA
GGACTGGCATCTCTGCCTATCCCCCACCCCTTGTGTACCACATCTTCCTGCTATACCCTACACTTTGCCCATGGGAGCTGAGCCCCAGCGAGGG
AGGGAGGCACAGAGGAAGCCCCTTCGGCGGGAAGCAGGTGTTGTGAGGCCGTGAGGAGTCCTATGTCCCAAGGCGGGAGGGAGGCAACTGGAGC
TTTTCAACTCGAAGGGCTGAGCAGGTGGCCCCTCTGCAGCTGCTTTCTCTGCCTCCATAGCACTGATACAACTCCCGGCCACCCCTCCACACTC
CCCTCCTCTGTGAAACAAACACAGCTTCCTCACACCCTTTGCTGAGAAGCATTTGGCATTTCACTTCCCCTTCCATTTTGCAAGAGGGAAACAC
CAAGCTGGAGCTGGTTGGAGAAGTAGTGGAAGCACCAGGTACCTCTATCTGCAGCCCAGGTACATGGTGCTTGAATGCAGAAAAGCCTCCTGGG
CTGACCTACTTCTCTTTCTCTCTTCCAGCACAAATTGAAGTGATCCCTTGCAAAATCTGTCGGGACAAGTCGTCTGGGATCCACTACGGGGTTA
TCACCTGTGAGGGGTGCAAGGTGAGTCATAGGCATGTGTATGCCTGCATGTGTGCGTGTGCATACACAAGCGCGCGCGCACACACACACACACA
CACACACACACACACAGTGTCTCCTTAGAGATAAACAAGGGGGTTAATGGCCTTTGTTCTGACTCCAGGGATGATCTCCTGGGCAGCCAGGAAA
ATGCCTGAGTAGCGCCTTCCTGCAGGGCCCTCAACACTGGCAGGGCCCTGTCTTAAGCTGGGGAAATGACTACAGGATAAATTGCAATTACACA
AATAGATGGAGGAGAGAGAAAACTGACAGGTCGTGGGATGTAGAAAAGCTGCCAGAGCTTGTGGGCTGGAGGCCTTTGTGAGTGAGCTGGGCCT
GACCAGGATAAGCAGTCTTGCCCTCCACCTGCTTTCCCCAGGGCTTCTTCCGCCGGAGCCAGCGCTGTAACGCGGCCTACTCCTGCACCCGTCA
GCAGAACTGCCCCATCGACCGCACCAGCCGAAACCGATGCCAGCACTGCCGCCTGCAGAAATGCCTGGCGCTGGGCATGTCCCGAGATGGTGAG
GCCAAGTCGACAGCCCCCTGGGGTTTTCCTGGTGTCTCCAGAGGGGCAGCCTGGCCTGCTGAGCTAGACAAGGCTTAACCTGCAAGACGGCCCA
TCCTCTGGTCTCCTCTCCATTCCTCCCCGTTACAACCCCTTGTCTCCTCCCCTCCAGGGATGGATGGGTCACCCCCATCAAAGTTCTTTGGAGT
CCCCAACTTTAGGCTTTGCTAGAACAAACATCCTCTTTCCAAGGTCCCATACCTCTTCATCTCTTCTTACTTGGAACGAATTCTCCTCACTCTG
CTGCCCTCTGGCCCAGCTCCCATGCAGCTCTGGCACCTTCCCCTGCTACCCTGTTGTTGTAGTTCTAGCTCTATCTCCTTTTCTAATCCCCCAT
TCCCATACTTGGACACAGGACTATAGCCAGGAATGGAAACAGAATTGGCCTGAGAACAACCAGAGGGTGGTCGTGGGGGAGGGCTGGTGTTCCT
GGTGCCTTATCCACCCTCCTCACCCACCACCTCCTCACCAGTCTCCCTGCACACCTCCACCACCACAGAAGGAGCCTAGGGTGGAGCTGGGGGG
GCATGAGGTGATGAGGAGCCAGAAGGAGCCCGTCAGCACTTTTCAGTGCCCAAAATAACAAAGCAAAAGGAAGCACGCAGGGGTGCAAAGGGGC
AGGCGGGGCGTAGGCTGTGCCCCTACACCTGGGAGGGGTGGCGGGGGGACTAAAAAGGCAGGAAAGAGAGAGCAGAAGAGGATGTTCAGAAACA
AGCCGCGGAGCCCGGGTTGGGCTGTGGTGAGTATCTAGGTCACCAGGGAGCCTGCAGGCCTGACCACAGGGAGACCTGTGTTCTCAGCTCTCCT
CTTCCTCCGACCCTCCCAAAGAGGCGAGGTGACCCCAATACAGCTTGAGGCCCTCTGCTCGACCACCCCCAGCCCCAAGATCTGGAGGCCAGAA
TCCCCAGCCTAGACTCATTGCTTGAATTCTGCCATGATTCAAATCTGATTTAGAACTTAGAAGATTTTGCTTAGCAGATTGAAATGGCAGACTG
GACAGACCCCAGAAGCAAACAAGAGTGAGAGGTGGACGGGGTCTTGAGCATAAGTTCCCTAAAGGCTAGAGAAGCTGTGCTTGGAGTCAGCCAT
TCAGAGAGCAGCAAGTTAATCCTTTAATGACCAAATGCCTCCTGACCCTGCCCTGTGCCATGTTCTCCTGCCTCATAAACCCCTGGTCCCTGGA
CCTCTTTCAGCTGTCAAGTTCGGCCGCATGTCCAAGAAGCAGAGGGACAGCCTGCATGCAGAAGTGCAGAAACAGCTGCAGCAGCGGCAACAGC
AGCAACAGGAACCAGTGGTCAAGACCCCTCCAGCAGGGGCCCAAGGAGCAGATACCCTCACCTACACCTTGGGGCTCCCAGACGGGCAGCTGCC
CCTGGGCTCCTCGCCTGACCTGCCTGAGGCTTCTGCCTGTCCCCCTGGCCTCCTGAAAGCCTCAGGCTCTGGGCCCTCATATTCCAACAACTTG
GCCAAGGCAGGGCTCAATGGGGCCTCATGCCACCTTGAATACAGCCCTGAGCGGGGCAAGGCTGAGGGCAGAGAGAGCTTCTATAGCACAGGCA
GCCAGCTGACCCCTGACCGATGTGGACTTCGTTTTGAGGAACACAGGCATCCTGGGCTTGGGGAACTGGGACAGGGCCCAGACAGCTACGGCAG
CCCCAGTTTCCGCAGCACACCGGAGGCACCCTATGCCTCCCTGACAGAGATAGGTGAGCAGCTGGGGAGGTGGAGAGGGTGGTAGAGATGAGGG
AGGGGTTTCCACCAGTACCCCGTATCAATCAAACATGCGCCTGAGGGAATTGAGGGGTCCAGACGAGGGGCGGAGGGAGGAGGCGGAGCAGGAT
AGGCCAGGCTGAGAAGTGCCCTTGCATGGGTAGGCGTAGGAGCTGGCTGAGATCAAGCCATGCCTTCCTTCTCCGGCCCCAGAGCACCTGGTGC
AGAGCGTCTGCAAGTCCTACAGGGAGACATGCCAGCTGCGGCTGGAGGACCTGCTGCGGCAGCGCTCCAACATCTTCTCCCGGGAGGAAGTGAC
TGGCTACCAGAGGAAGGTGAGGCCAGGAGACCTGCAGGAAGGGAACGTATCCCACCCCCACCGGGAGAGTTCAGAGATGGCTACCTGCGCACGA
CTGGGTCCTGGGGCAGGGCGGGACATCACAGACACAGGCTGGCCAACAAGCATGTGCACACCTTTTATGTACAGTTGCGAATGTGTGTATCTGT
CTACACCCCAGCAGATGGATGAGCGCTTTTCCTTAGTTCTTGCCTATTAAAATTGTACCTGGCCTTCATCCTACCTCCTCTACCATGCAGACCT
TGGTGCACCCTCCTTCCTCCTCTTCCATCAACAATAACAATAATCAGAACCCTGATTACCATTTGTTAAACACCCCTTCTCTGCCAGGCATTGT
GCTAAACGCTTTATGTAAATTATCCTCAGCCCCTACCACAACCAACCTATGAGGGTGGCATTTATTCCTACTTTTAACAGACAGAAACTGAGCC
TAGAGGGGTTAATACGTTTCCTCAAAGTCACAAAGTGGTGGAACCAAAGTCAAATTCAGATTCATCAGGCTCCAAAGTTTATGCTGCCTTTTCG
ATCACACTCTCATACCACCTGCTCTAAACACACTCGTTTGGCACTTCACATTTGCTTCTCCAGGTTATTGAGACCTTGGGCATAACCTTTGTGG
GGGAAGGGGCAGCTGTATGTCCTGTTCACCTCAACGAGATATACCTTGTTGGAGGGCAAAGCTGTATCTCATACTTTTTGGATTCCCTCACAAC
AGCTAACTCGGTGTCAGCAGCCGGTAGGTGCTCAGTGTGTGGGACTCACTGGCAGGAATCTGTGCATTTGTGCTAAGACCAGGCTTTTGAAAAT
GCTAGTTGAGAACATAGGAGTTCAGAGCCTACCCCTTGCAGTTTATTAGGTGGGGCTCCAGGGCTCAGGAGGATCACAGGGCCACACAGAGCGC
TACAGCGGGACCCTCCTCCCTCCCTGCAGTCCATGTGGGAGATGTGGGAACGGTGTGCCCACCACCTCACCGAGGCCATTCAGTACGTGGTGGA
GTTCGCCAAGAGGCTCTCAGGCTTTATGGAGCTCTGCCAGAATGACCAGATTGTGCTTCTCAAAGCAGGTGCCCAGGGATGGGTGGGCAGGCCT
GGGGACAAGGGGACAGAGCCAAGTGGAGGGAGGTGGCTTAAGGAAATCAGGGGGACAGAGTCAGATCCTGGCTTTGCTTGACACTGTCCCTGCA
TCTTCTCTCCCCACTGCCCAGGAGCAATGGAAGTGGTGCTGGTTAGGATGTGCCGGGCCTACAATGCTGACAACCGCACGGTCTTTTTTGAAGG
CAAATACGGTGGCATGGAGCTGTTCCGAGCCTTGGGTGAGGGGCAGGGAGAAATGAGAGGGAAGATTCTGATGCCAACCCCAGGCAAAGCTTTG
TGACCCAGGGCACCCTCTTTTCAGGGCGAATTGCCCCCTCTGCTCTAAACACAATAAGGGCGGTGTCCTCGGGCACCATCGCTCCAGCCACTCT
CTCACTTTTCTCATTTCCACTCCATCAGGCTGCAGCGAGCTCATCAGCTCCATCTTTCACTTCTCCCACTCCCTAAGTGCCTTGCACTTTTCCG
AGGATGAGATTGCCCTCTACACAGCCCTTGTTCTCATCAATGCCCGTGAGTGTTGCTGGGCTTGGGTGAAGGACATTCAGGTGGCACGGGCATG
GCAGATATTGAAGAAGAGTCTAGACCTTCAGATGTAGTTAAATCTGGGAAATTGCTTTAAATAGCAGAATGAGCCCTACTCAGTATTGCTATAA
AATAAAATGAGTTAAAATAAAGATTCAGAGGACTCTCAGAGAGGGACAAGAGCAGCATAAGGTGGGGTTGTGGGAAGTGGGGAGAAATGAGGTT
GAGAGGAAATGAGCCACTTTCCTGACAGAAATGTGTCTGATTGTTAGTCTATGGCAGTGATTTCATTGTAGCACACATCAGAATCACCTGGGGA
GCTTTAAAAACTATTGCTGCCTGGGTCCCACTTCCAGAGATTCCAGTGTACATGTGCTCCATGACTTATGATGGGGTTATGTCCCAATAAACCT
ATCGTAAGTTGAAAATATGGTAAGTTGAAATTGCATTTAACACGCCTAACTTACTGAACACCATAGCTTAGCCTAGCCTACCTTAAATGTGCTC
AGAATGCTTACATTAGCCTACAGTCTGGCAAAAGCATATAACACAAAGCCTATTTTATAATAAAGTGTTGAATAGCTCATGTAATTTATTGAAT
ATGGTTCTAAAAGTGAACAGCAGGATGGTTGCATGGGTATTCAAAGTATGGTTTCTACTGAATGCAAGTGGCTTTCTCACCAACATAAAATCAA
AAAATAGTAAGTCAACTCATCATAAGACTGGGACCATCTGTAATTGATAAGGGGTGCACTGAATTTTAACAGCCACCCCCAGGCCACCAAGATT
AAGAATCACTGCTCTGTGTGAACTAATTTTAAGGCTGTATGCCTATAATAGGAAGACTCTGGATATCCTATCCACTCCCCTGGCATGGAGTAGC
TGGGCTGAGCCAGATGAATACTAAATATTCAGAGAACCTAGGGAAGTGGGTCAAGCTGCTAACCTGAGTTTGCAGATCGAGACTATCAGTCTTC
TGGCTCTGCCAGTGAGTGGACACCTAAATATGCCCTTCAAACTAGAAGATAAAAACTTTAAAGATGACTTCTGGATATTTTATAGAATCAGGGC
AAAAGTTAGTCACAGATTGCTGCAAGCCCCCTGGTGCAGGCCTGGGCACCTTCAGGAAGGCCACCTCCTATCAGGAGCCCTTTTCGTACATGGG
GGAGCTCTTTTCCAACTCTTGTTACCCCTATGACAGAATCCTAGTGCCTAGCATTGAATGAGCTATGTGGAATATCAGGTTACCAGGGAGAAGG
TAGCTTGGAGTCAGGAGATCAGAAATTCTCATACCTTTCATCTCTTCCCAGTTTTCCTTGTAGACAGCTAGCCAAGCCCAACGTCCAGAGCTTA
CCATTATCAAACCAAGGCACACACATGCACGCGCTCAGCTTAGAAGACCTCTATCCAGCACAGATGTCCACAAAGATACACCCTTTTGTTGGGA
GTTAATGTCCATGTTCTTTCTTGTTCTCATTACGGTCCCACCCCCTCCTCCAGATCGGCCAGGGCTCCAAGAGAAAAGGAAAGTAGAACAGCTG
CAGTACAATCTGGAGCTGGCCTTTCATCATCATCTCTGCAAGACTCATCGCCAAAGCATCCTGGCAAAGGTAGGAGCAGTCCCTGGGGTAGAAG
AGGCCAGGCCCATCGCTAGCTCTGTAACATCAGAGTTTGCGAGGGCCGGGGTCTGTGGGTACAGAGGAGGGAGTGCGGGAGTACCACTCTCTGT
TAGAGAGCTTGCATCAGCAGTGGGAACTAAGGGAATGAACAGCTACTTCCACGTGCATAAAGACTGGAAAGTTAGAGGGCCTGGGATTGGGAGG
GACCTCCAGGGAACAATTCAGTTTAATATAGCCAGCACTTACCCAGCACCTGCTTGTACAAGGCACTGTGGAAAGACACAGAGATTTGGTCGCT
GCCCCCACCAAGAGATTTTAATCTGGTATGAAGAAGAGATCTGTGTATCACTAACTCTAACATAGAGTAGAATGTGGTATGTGATATAATAATA
ATGCAATTAACAGAGTGCTTTTGCTGACATGCTTTCTCATCCTCATGGCAACCCAGTCAATAGGACAGGTGTTCAAATCTCCCTGTGTAGCAGC
CGGGCACAGTGGCTCACGCCTGTAATCCCAACACTTTGGGAGGCCAAGGCGGGTGGATCACCTGAGGTCAGGAGTTCGAGACCAGCCTGACTAA
CATGGTGAAACCCCATCTCTACTAAAAATAAAAAAAATTAGCCAGGCATGGTGGCAGGCGCCTGTAGACCCAGCTACTTGGGACGCTGAGGCAG
GATAATCGCTTGAAACCGGGAGGCAGATGTTGCAGTGAGCCAAGATTGTGCCATTGCACTCCAGCCTGGGCAACAAGAGCAAACTCTGTCTCAA
AAAAAAAAAAAAAAAAAATCTCCTTGTAGCTATCAGGAGACTTCAGTGACTTAAATGCAAGATTGAATCCCAGTGCTCTTTGCGCTCTTTCTAT
CCCTGTGTCCCCTATGTATAACTATAATAAGTGACACCAGGAAAATGTTATGAGAGTATAAAACAGGGATTAAAAATAATTTGGGGGTAAAAGG
AGTGGGTCATAAATACTTCCCAGGGAAGATGACATTTATACTAGGCCATGAATGATGTAAGATTTTAACAGGCATTCATGGGGGTGGGGCAGGC
ATTCCAGGCTTAGGGAACAATAGGAGCAAAACAAAAAAAATGAAAAAAAATCCTTTTCCTGAGGTTTAACCAAAAAAATGGATGAGATGAGTAT
GAGAGGCTGGGGATAATTGTTTTATGGGATTTGGGTGTGGGACTAGGGTACAATGAAGACCAAGAACAACAGGAGAAAAATAAGGAGGCAAAAT
AGTGTGTATGTGGAGAATCACTCATGGTACATCCTCACTAAAGTGTAAAATCAGGAGCTGGGATAGACTGGTGGGGCAGAAGAGCACCAGATGA
TCAGCCTGAAAATTAGGTCAGGGGCAAATCAGAGAGGACTTTCAATGCCATATAAGAGTTAAAGCTTTATTTCTAGGCCACAGGGAGCTCCAAA
GGCTAGAAAAATGACACAATCAGAGCTGTCATTTAGGCAATTTACTTTGGAACCAGTATAAAGGAACCATTTATGTAATTATTCAAGCAGGCCT
TTGCTATGTGCCAGGCACAGGGCTGGGTGTTAAGGATACAGCAATGACTTACACGGTCTGTGCTCTCAAGAACTTGAACTTTAATCTGCTACAG
GATGGATTTGAAGGAGGAGAGACAGGAATCTGGGAGAGCAATTGGAAAGCAAATCCGGTTCGATCTGCTTTCAAAATACATCACCTTCCCTACT
ATTACCACCACCCTGGCCCCTACCAGCTCTCAGCTTTCACTTGGACTTTAAGAGAGGCCTCCTAACTGAGCCCCTGTTCCACCCTCATCTGCCC
TGTAGCAACCACACCTACTCCGCACCCACGGTAGCCCTTTTAAAAATGCAATCTCATCATGCCCTACTCCTGTGGTTTTTTTCTTGTTTTTGTT
GTTGTTGTTTGTTTGTTCTGTTTGTTTGTTTTGAGGCAGACTTGCTCTGTCGCCCAGGCTGGAGTGCAGTGGTGTGATCTCGGCTCACTGCAAC
CTCCACCTCCCAGGTTCAAGCGATTGTCCTGCCTCAGCCTCCTGAGTAGCTTGGATTACAGGGATTACACGCCCGGCTAATTTTTGTATTTTTA
GTAGAGACAGGGTTTCACCATGTTGGCCAGGCTGGTCTCGAACTCCTGACCTCAGGTGATCTGCCCACCTTGGCCTCCCAAAGTGTTGGGATTA
CAGGCGTGAACCACCACACCTGGCCTTGACTCCTGTTCTCAGCCCTCCTGTAGCTGCCTGTGATGCCGAGAATCAAATCTAGAGTCTGCGTCAT
GGTCAAGTGGCTCATAACATGATCCCTGCCTTCTTTTCTCACTTGATCTTCCACTGCCCCTTCAAACACCCATTGCAGCCACACTTGCTTCCTT
GCTATTCCTCGAACACATCAAACCCAGTCGCAGGGCTTTTGTACCTGCTATTGTAGTCACCTGGAGGGTTCTTCCCCCAGTTTTCCAAATGGCT
TACCCCATCTCTTCATTCGGGAGAGGTTTTTCCTGACCAGTAACCCCATACAAAAAGCTTTAGTTTTCTTTAAAGAACTTATTATCTGATACAC
TACATATTTATTTTCTGGGCCCCTCACCAGAATGTAAATTTAATCAAGGTACAGATTATACTTTATTGACTGATATATCTGATATATCACTAGA
GGCCATCACAGTGCCTAGCTCAGATCCAGATGTGTTCTCAACAAATATTTGTTGAATGAATGAAGGAAGCTATTGCCATAGCCCAAAAAAGCTC
AGAATAAAGCAGTGGTGAGGAAGAGAGAGAATCTAGGAGATATGAAGGGATCACACCCTGCTGCCTGCTTTTCTCTGACTGCCTCTTTCCAAGG
AAACTAAGCTGGGGGAGGGAGGCAATGGCAGGCAAGAGACATTTTTAAGCCTCTTGGTTGTAGAGGAGACTGCAAGAGATAAATTGTTCTTTCA
GCCTTGCTCCAGTGAGGTCTCCCTGCCTCCGTCTGCTCACTGGTTTCTGTGCCTTTTTCATCTCCCCTTTGGCTGAGTGCTGGCAGCATTGGTT
TGCTACTTGCAGTGTTAGGTGCCTGCTTAGAAAGTCTGTTTAGTTCAAAGAGTTATTAAGCATGTGCCATGTGCTAAGCATTATGATAGGTACA
GAGGAGACAGGGAAAGAGAGACCTCAGCCAAGGAGCTGAAATCTAGGGTGGGAAGGCAGACAAATTGGACCATTTTCCTGCAATGTAGTAAGTG
CTACACAGAGGAAAGCCCAAAAGAAGGGCCCTTAATCCAGATGGGAGGCAGTTAGGGAAATAGTCTTAGGAGAGGTGACACTAGAGGGTAAGGA
TTAGTAATGGGAAGAGGGGAACAACATGCACAAAGGCAGAGGCATGTGACAGCATACAACAAGAGTCACAGACAATCACTCTTATAAAAGCAGG
AAGTGCACATTAGAGTCTTTGACAAGGTTCATTCTAGAAGTATTGGGAACATAAATTGAGGGCTTCACCCAGAAAACATTCACCTGTGCCCCAC
CCACTCTCACTTCCCTCCAGTGTCCTGAACACACACGTACTTCTACCAGTGGGATTTGGCTGGTGCCAAAGTGCCAAGTACATAAGGGGAAGGC
AAGGAGGGTTTGTCCTAGCCCAGGAAGAATAAGCGGACTTCTTTGCTCTGAGGAGAGCTGAAGTATTGACCCTCCCTTCCCCATTAACCCATAT
CCAGCTGCCACCCAAGGGGAAGCTTCGGAGCCTGTGTAGCCAGCATGTGGAAAGGCTGCAGATCTTCCAGCACCTCCACCCCATCGTGGTCCAA
GCCGCTTTCCCTCCACTCTACAAGGAGCTCTTCAGCACTGAAACCGAGTCACCTGTGGGGCTCTCCAAGTGACCTGGAAGAGGGACTCCTTGCC
TCTCCCTATCGCCTGCTGGCCCACCTCCCTGGACCCCGTTCCACCCTCACCCTTTTCCTTTCCCATGAACCCTGGAGGGTGGTCCCCACCAGCT
CTTTGGAAGTGAGCAGATGCTGCGGCTGGCTTTCTGTCAGCAGGCCGGCCTGGCAGTGGGACAATCGCCAGAGGGTGGGGCTGGCAGAACACCA
TCTCCAGCCTCAGCTTTGACCTGTCTCATTTCCCATATTCCTTCACACCCAGCTTCTGGAAGGCATGGGGTGGCTGGGATTTAAGGACTTCTGG
GGGACCAAGACATCCTCAAGAAAACAGGGGCATCCAGGGCTCCCTGGATGAATAGAATGCAATTCATTCAGAAGCTCAGAAGCTAAGAATAAGC
CTTTGAAATACCTCATTCCATTTCCCTTTGGGCTTCGCCTTGGGGAGATGGATCAAGCTCAGAGACTGGCAGTGAGAGCCCAGAAGGACCTGTA
TAAAATGAATCTGGAGCTTTACATTTTCTGCCTCTGCCTTCCTCCCAGCTCAGCAAGGAAGTATTTGGGCACCCTACCCTTTACCTGGGGTCTA
ACCAAAAATGGATGGGATGAGGATGAGAGGCTGGAGATAATTGTTTTATGGGATTTGGGTGTGGGACTAGGGTACAATGAAGGCCAAGAGCATC
TCAGACATAGAGTTAAAACTCAAACCTCTTATGTGCACTTTAAAGATAGACTTTAGGGGCTGGCACAAATCTGATCAGAGACACATATCCATAC
ACAGGTGAAACACATACAGACTCAACAGCAATCATGCAGTTCCAGAGACACATGAACCTGACACAATCTCTCTTATCCTTGAGGCCACAGCTTG
GAGGAGCCTAGAGGCCTCAGGGGAAAGTCCCAATCCTGAGGGACCCTCCCAAACATTTCCATGGTGCTCCAGTCCACTGATCTTGGGTCTGGGG
TGATCCAAATACCACCCCAGCTCCAGCTGTCTTCTACCACTAGAAGACCCAAGAGAAGCAGAAGTCGCTCGCACTGGTCAGTCGGAAGGCAAGA
TCAGATCCTGGAGGACTTTCCTGGCCTGCCCGCCAGCCCTGCTCTTGTTGTGGAGAAGGAAGCAGATGTGATCACATCACCCCGTCATTGGGCA
CCGCTGACTCCAGCATGGAGGACACCAGGGAGCAGGGCCTGGGCCTGTTTCCCCAGCTGTGATCTTGCCCAGAACCTCTCTTGGCTTCATAAAC
AGCTGTGAACCCTCCCCTGAGGGATTAACAGCAATGATGGGCAGTCGTGGAGTTGGGGGGGTTGGGGGTGGGATTGTGTCCTCTAAGGGGACGG
GTTCATCTGAGTAAACATAAACCCCAACTTGTGCCATTCTTTATAAAATGATTTTAAAGGCAAGAAGTGTGTGTGTCAGAGGGTGGGGGAGATT
CTTAAATTAGATTACCTGCATGCCTGCTCTCCAGTCTCATTCCTGGGTCAAGACTCAGGTTTCCAGCTCAGCAATCCATCAGCATTATACAGAT
CCAACCCACCCTCACCCGACCCCTGCAGTTTCTCCCCAGGTGGAGCAGTCCCTCAGTGAGGACTGTGAACGAATCTTCAGGAACCCCCACTGTA
GGAGCCTCAAACTGAGCCCCACGGGAGATGCTCTAGACTGAGAACTTCCCATAAATGATACCCACGGGGAACGTTTAGATTTAGAGGTTGCACA
GAATTGCTCCACATCTGGGAGACCAAAAGACAGTCCTCTGGAAGGTGGCTGGCCCAAGCTCCCCAGTGGGGGAATCAGGATGTCAGAGAGATCC
TCTAGAACCTGCTGTTCTTGCTATTGCATGACCCCTCCCTGGCACCAGAGCCTCCCTCCTGGCTCCCTCCCCTGTCACTTGCCAGCCTGTAGTG
GTGCTTGCTGCAGCCCTCCCTGGTTGCTTTATTTATTTATTTTGCACCAACAGGGTTGCTGCAGACTCATTCTTGCCTGGTTTAAAAAGAGAGA
GAGAGAGAGAGGAAAAAAAAAAAGGAGAAATGCTTTCTGGCTCTTTTCTCCACCTCAGTCTTGGCAGCAGCGGCCGCAGCAGCAACAGCAGCAG
CAGCAGCGGCAGGCAGCAGCCGGGCAGCCAGGCAGCGGGGGTTGAGGCACACAGGGAAGGTGCAGGGGCCTGAGGTGCAGCTCGAATGGGACAG
GGCCCCCAGCGCTGGACAGATGCAGTGCCAAACTTGATGCCACCTTCCAGCTTCTCCGGTAAGTGCCCCCACTCTCTGTCCCAAAGATGCAGCC
GCCCTTTTCCATAACATTCTCCGAGACAGGCCAGACTAGGGGCCAGACAGGCCCCTCAAGGCAAGAGGGTTTGGGCCCCCACACTGCTAACAAT
TAATCCTGGCCCCATGGGACTTTGAGGGGGAAACTCAAATATCCCAAGTACCCCTGGGTGGAAGGGCTCCAAGGAGACCTCCCTTCCATCTCCC
CAGTGCTTCCCCTTCCTCTGGAAGGGTTTTTCTCCACAATCGGTGTGGATCTTCCAGAAATCTTTCCCCCAGGAACCCTCTCCCCACACAGTTC
CCATTTGTCAGTCAGGTGTGCTTTAAAGAGGATACAGGATACCCAGGTTCAAAAGTGTCTCTGGACTGCCACATAAAGACAAGAAAAAGGCTCC
CTACCCTTGAGGGGTGATGCCTTCAAGGGTCATGTCTTGGTGATGTCCCCACCCCACTGAAGGGACAAAAAAGTGGTTCTGACATCTCGCTTCC
TGCAAAGTGGCCGGAAGCCGGTCCTGCAGCCATGGAGGTTGGCAAGGGAAATTTGTGATTAAATTAGCCACCTTAGAAATAAAATAGGTTGCTC
CAGCTCCCTCAGCCCCTGGCCCAGCGGACTCAGGGGAAACTCACAGGAGCAGGAAGGAAGAAAGCTGGGTTACATGCTTCACTGCACTTTTGCT
GAATGCAGAGCGAGGATGGGCAGGAACTGCAGCAGTAGTGATGAAGAGCTAGCTGTCAGGTGGACTTCCCAGAACAAGCCTAGGGAGAACAAGG
GAGGCTGGGCCATCTTTCCCGGGAGAGCTCTGGGCATGCCACAGAGACCTGTTAGGTCAGTTTGGGGCGAGAAGAGTCTGCTCTGCAGTCTTTG
CCCCACGTTATTCTTTTTCATCCCCAAGCCTCACACCAGTTTTCTGCATCCCTCTGCTCTTTTGGCCTCTTTCTTTAGTTTCCCAACTAGGACA
ATCCTCTCAAGTGGCTGTAGGATAGCCACTAGAATGATCCTTCTAGGAGGTGGAGAGTGGGAAGGAAAGGGGAAGAAATGACCATCTCTTAAAC
TGCCTCTCCTAAGTTCCACATGAGAAAAAACAGGGCATGTAGTAGAAAGCTGACCCTGTAGAGCCCAGAGAGCCTGAAGCCCACAAGCCCAAAG
GTGATCAGGATTGCCGCTCAGCAGAGACTCAGACGCGCTGTATCCCAGAGAGCATCCATGGGCTTTTGCTCCTATCTCTGTAGCCATCCCTTGC
CAATTCCAGTACTTCCTCTGCCTTGGGGTCCCTGTTGACATCTAACAGGATGAGTCAGGGGCCCTCATCACCTAGAGGGCCCCTTCTCCTCTGT
CACCTCAGCCATTGTAGTCACCATCTTCCTGAGGGTTCCCGGAACCTGGTACCCAGAAACTGACTATAAGTCTACAGGCTCTGCACACTGTCTG
TGCCCAGATACCTGCTGTGCCAGCCAACAGCTCCCTTCCTCCCCACCCTACAGCACTTGGTCAGATGCTGTCTCTCCTCACTTATCTATGCTCC
CTGGTCCACAATCTGTCTCTTGGGAATTTCTCAGGCAGCTCAGGCCAAAAGGGACAAGAGCTCTCCCTCAGATCCACACACTGGACCAGAATCC
AAACACCATTAAGGAGGGATATGAGGGAAGCCCAAGACTGAAGACCAAGCAACAGAACTCAAAACCTGGGCATCCTTTGGGTCTCTCACACAAA
CCAACTTCAATTGCAGTAGAGAAGCAGTTGCCCCTGGGCTCTTGCAGGGGATTCCCAGCTTCCCAGTCAAGTGCCTCCTGCATCCTATGCCACA
GCTAATGTACAGCTTGGCAGTTGTCCACACAGGCATTTTGGGGAGATTGGATCTTGTTAGTCCAGGGCTCAGGCCCTGGGCCAGGCTGGAAGAG
GGCTCAGCATGGGGGTGGAGCTGAGGCCTGCATGTTTCCCAGCCTTGGGTGAGCTAGTCAAGGTGAGGACCTCTCCAGTCAGGACAGGGAACTC
TGGGCCAAGGACATGATCTCCCTGAGCCTTAAGCAAAACTATGGGTACATAATGTGAAAGAATAAGCGTGAGCAGAGAGGGACAGAGCAGAGCT
TGGGAAGAGCAGGTATCAGGGAGAAGGGACCTGAGATCCTCCTGGATCTCACAGACATCAGGAAACCCCATACAGAAAGACTCAGTACCTCCCT
GCTGTTCCCTGCCCCATTCCCATAAGCTTTTTCCCCACAGAAATCAGGCTTGGCTAGGGTTCCATGAGCCAGTAAGCACTTGGCTGGTTATCCA
GGGCTGGAAGAGGGAGGAAAGCAGAGATTCCCCAAAGAGAAGCTCCAGGAAGCCCCAGGGAGGTGGCACACAAGAATTCTTCCTGGTTCTGTGC
CCTACTTATTGGCAATGCTGAGACAGATAGACCAATTTCTCTCCTCTCTAAGAAGGGCCAGGAAGTCCAGGGTCACCGTCATCTGAAGCCACTT
TCCAAAAAGGGCCATGTCATCATCTATTCCAGGACTGAAGAGGGAATGGATGCAGCCACAGCTCCAAAGCAAGCCTGGCCCCCATGGCCCCCGC
TCCTTTTCCTCCTCCTCCTACCTGGAGGGAGCGGTGGCAGCTGCCCTGCTGTGTGTGACTGCACCTCCCAGCCCCAGGCTGTGCTCTGTGGCCA
CAGGCAACTGGAGGCTGTACCTGGAGGACTCCCACTGGACACTGAGCTCCTGGACCTGAGTGGGAACCGCCTGTGGGGGCTCCAGCAGGGAATG
CTCTCCCGCCTGAGCCTGCTCCAGGAATTGGACCTCAGCTACAACCAGCTCTCAACCCTTGAGCCTGGGGCCTTCCATGGCCTACAAAGCCTAC
TCACCCTGAGGCTGCAGGGCAATCGGCTCAGAATCATGGGGCCTGGGGTCTTCTCAGGCCTCTCTGCTCTGACCCTGCTGGACCTCCGCCTCAA
CCAGATTGTTCTCTTCCTAGATGGACCTTTTGGGGAGCTAGGCAGCCTCCAGAAGCTGGAGGTTGGGGACAACCACCTGGTATTTGTGGCTCCG
GGGGCCTTTGCAGGGCTAGCCAAGTTGAGCACCCTCACCCTGGAGCGCTGCAACCTCAGCACAGTGCCTGGCCTAGCCCTTGCCCGTCTCCCGG
CACTAGTGGCCCTAAGGCTTAGAGAACTGGATATTGGGAGGCTGCCAGCTGGGGCCCTGCGGGGGCTGGGGCAGCTCAAGGAGCTGGAGATCCA
CCTCTGGCCATCTCTGGAGGCTCTGGACCCTGGGAGCCTGGTTGGGCTCAATCTCAGCAGCCTGGCCATCACTCGCTGCAATCTGAGCTCGGTG
CCCTTCCAAGCACTGTACCACCTCAGCTTCCTCAGGGTCCTGGATCTGTCCCAGAATCCCATCTCAGCCATCCCAGCCCGAAGGCTCAGCCCCC
TGGTGCGGCTCCAGGAGCTACGCCTGTCAGGGGCATGCCTCACCTCCATTGCTGCCCATGCCTTCCATGGCTTGACTGCCTTCCACCTCCTGGA
TGTGGCAGATAACGCCCTTCAGACACTAGAGGAAACAGCTTTCCCTTCTCCAGACAAACTGGTCACCTTGAGGCTGTCTGGCAACCCCCTAACC
TGTGACTGCCGCCTCCTCTGGCTGCTCCGGCTCCGCCGCCACCTGGACTTTGGCATGTCCCCCCCTGCCTGTGCTGGCCCCCATCATGTCCAGG
GGAAGACCCTGAAGGAGTTTTCAGACATCCTGCCTCCAGGGCACTTCACCTGCAAACCACCCCTGATCCGAAAGTCGGGGCCTCGATGGGTCAT
TGCACAGGAGGGCGGGCATGCGGTTTTCTCCTGCTCTGGAGATGGAGACCCAGCCCCCACTGTCTCCTGGATGAGGCCTCATGGGGCTTGGCTG
GGCAGGGCTGGGAGAGTAAGGGTCCTAGAGGATGGGACACTGGAGATCCGCTCAGTGCAGCTACGGGACAGAGGGGCCTATGTCTGTGTGGTTA
GCAATGTCGCTGGGAATGACTCCCTGAGGACCTGGCTGGAAGTCATCCAGGTGGAACCACCAAACGGCACACTTTCTGACCCCAACATCACCGT
GCCAGGGATCCCAGGGCCTTTTTTTCTGGATAGCAGAGGTGTGGCCATGGTGCTGGCAGTCGGCTTCCTCCCCTTCCTCACCTCAGTGACCCTC
TGCTTTGGCCTGATTGCCCTTTGGAGCAAGGGCAAAGGTCGGGTCAAACATCACATGACCTTTGACTTTGTGGCACCTCGGCCCTCTGGGGATA
AAAACTCTGGGGGTAACCGGGTCACTGCCAAGCTCTTCTGACCTTTCCTTCCCCAGTGGGGAACCCACCAAGTCCGCTTCAGATACCAAAGGGG
AAGACAGAACCAAGGCTGCTTGAACCAGAACCTAGTCCCGAGCAGCACCGCTCTCCTGCACCTCCCGCCTGCGTTGTGCCTCCTGCCGGAGAGT
CTGCTTCCTGAGCTTTTCCGGTCTGAGGATAGCATTGTCATTTCTTCTCTGAGGGTCCCAGGGAGCTGCAGATGCAGACCCCGTCGTTAGTCCA
GCCCCCGCTTCACCCCCTCCACACACAAAACAGGAAACATAATCAAAGCGCTAGTCAGCTAGTCTAACAACTAGGCTTTCTTCACACATGCTTA
TATCCTTTAATAACCAATTGCCAACCACGGCTATAAGATTATTTCAGAGGTGGGGCTGGGAAGTGCCACTTGCTCCTTAGAGTCTGTTTGTCAA
CCAGGCAGAGTCCCTTTCTTTTCTGCTCCCCACCCCAACCCTGCCCCTATGTACAGGAATAAGAGCAAAGGACCCACAGGCTACAGAGAAGAGG
ATGGGCACAGAGTGTGGGATGGAGAGGACAGACCATATACTGCACTCTGTTTGCATGAGCCTCTACCACCTTCCTCTATCTACCAGATCATTAA
ACCTGCTGTCAAAGGGCCACAACAGTAGCAGCCAAAACTAAATGTCATCTCTGGAGTTTTCTTTACTTCAGTCTATTTCCTACCCTCATTTCTG
TTATATCTCCCCAGCTCCTTCTCTTTCTGCTTGCCCATTGATTATGTGTCCCAATGGCATTGCCTCCATCTAACCTGCCTGACAAACAGGGTAA
GGAGTGCCCCTCCCACCTTCACTTTCCTCACCGCCCTGCACCCCCACCTCCATGCCCGGAGGGATCAGCACTCCTAGCCCCGGTTTCAGCCTCA
ATCCTTTCCCTTTCACTCCCCATCCCTGGAACTGGAGAAGGAGCGATCCTCTACCTTCCAGGGGACCCCTACATAGAAATTCCACCTGGGACAC
CCAGTTGCTGCCTCTCTTTCCCATTTCTCCATGGGAGCTCCTCATCATTTTTGCGTCACAGATCCCTAGTGCCCTTGGGGAAAACTCAGAACTC
CAAGATAATGACTAACAAACACAAGAATCCGCAGTTGTCAAAGGAGAGAGACCCAGGACACTGCAGAGACTAGGCTTGGAGGAACAGGGAGGAG
GGCACAGCAGGCTGTAGTGAGAAGTGGAGGGAAGGGGGAACAGCAGTAACTGCCTGCCTAGTTAATTTCCACCATCCTTCTGCAGCTTCCTCTG
GTTCTGTGCTTAGTGCAGCCCCAGTGGGAAGCTGTCTCCGGGTAGAGGTCACTGATTTACAGAGACCCCCAGATGGGGAGGTGGAGTAGGAGGT
GAAACTGCTGAGTACCACTGACTGTGCTTCAAATAGAGGGCAAAGCAGAGCCCAAAAAGAAGTAGAGATGCTGGAAGCTCAAGGAACAGAGCAA
CAGCCTCAGCATCACTGGAAGGGAGGAGTGACAGGGAGAGGAGAGGAGTCGGAGGAGGGACTGAGAAGGGAAGAATCACGTGGCCCTAGGGAGC
CTTTCTGCGCAGTAGCATCTACCCAGCGTCCTTAGTTTCCTCCCACCCACCCCTACACCAAACCAGATGCAAGCATTGTGAGCTTTCTAGTTGA
TTCCTGCTGCTGTTGTCCTTGCTCTGAGGAGACTCCACTCATTAGAAGATTCCCAGCTCAAACTGCCCGACAGATGAGACGCTCAGAGCCACTG
AGGAGGTGAAGCTGGACTTCACAGGCAGGCCGCTCTTTAGTGGGGCAGAGATAGGAATATGGGGGTAGTACCAGAGGTGAATGTGAGAACCAGG
CAGACAGGAGAGTGGGGCTGGAGAAACTGACCTGCTTGAGAAACGAGTTTCCCTGAGCCTGCAACCTCCCCACCCACCATGCACACACAACTCA
ATCAGCATCCCAGCAACTTCCCCTTCTTTAGTGTATAATGTACCAGACAGATTTCCTGGGGCACAGCCCTCCCGCTCCTTTCCATAACCTTCCA
CCAGCAGCTTATTAGGTTGGGACTTGGGAGTCAGACGCTGAAGGTTCTCACTCTAGTTGCCCTCTCTGCAATGCGGAAGCAGCAATGCTGTTCA
GAGAAATCTTCTCTGGGAAGCCCCTGACGCTCTGGGGAACAGATGACTGTCTGGTGCTGGGACATGCTGCTGCCCAAGGTCTCTGAAGTCTCAA
AGTCTCCAGAGCAGAGCTACCCAACAGACACAGAATCTAAGTCACACATGTAATTTCAAATTTTCTAGTAGCTACTAAATTAAAAAATCAAAAT
GAAACGGATACATTTAAATACTTACTTTATTTAACCCAATATACCCAAACTATTATCATTTCAATATATTATCAATATAAAAATCAATCATAAA
ATATTTAACATTTTTTCATATTAGGTCTTTAAAATCCAGTGTATATTTTACACTTACAGTACATCTCAACATCGCAATTCAGTTACTAAATTTT
CACCAGAAAAATCTATATTTCATACAGGTCACTGTTGAGAAAGTGGATTCACACATCTAAGTTTTTTCAAACATAAAACATTTTCCAATAACTG
AATCGAATCTCTGTCTTAAATTTTAAATTAAACAAATTTAAAATTCCATTCCTCAGCTGTACCACCTACCTTTACAAGCATTCAATAGCCACAT
GTGGCCAATGGCTACCATATTGGACAGCAAAGCTTCAGACATTGCAACCTGGCGTATAGACTAAGGTCTCCTTGGCAGTGGTGGGTGGACAAAG
TATCCCTCAAGAAACACATTTAATCTCAAGGAAATGCCTATGGAGTTAGTCCCACCACAGCCCCTTAGTCTCCTTTCACAGCTTTACCAGAACA
CTTTTGAGGGGTGAAGCCCATTCAGGAAACAAGCTTACTATGATGAGCACTTCCACAGCTTGTCCAAGCGTTAGCATGCCAGTCCCTCATCTTA
CCTGTCGGGAAGACTGCCCTCAGCTCAGCTGTGATGGCACAGGCTGCTGTGTGTGTGCTGATGAGTGTGCAAATGCAGCCAAGGACATGAGTGG
GTGGTGTGTATGCAGAAGTTTTGTGGCCCATGTGCAGGGATGTATGGTACCACATCATGGGGACAATCTAATGGAGGCTCTGCCCAGGGTGGGA
CAGCAGTTCAAAGAGAAGAAAATAACATTTATTGAGGGCCTATTCTATGTCAGTGGCTTTATATATATTTTCTTATTTAATCCTTACATCAACC
CTATAAAGTAGGGAGGCATCTTAGCTGGTGAAACTAAAGGTCAAAGAGAGTAAGTGCTCAAGTGCACAGAGCTAGTTAGTAAATAGTAACTAGC
TATTTGAGCTCATGGCTGTCTGATTGAAAACTCCCCCTTTCCACCACACTGCCATCCTCACTGCCATGCCCTCACGGCTCTCTCTCCCAGGGCC
TTTCAGTTGCAGGACACGACCTGTAAGAAAGGAGAAATCTTCCAATGCATCCACTCTGAACTTTCAGTGGGGACTGGGCTAAGTTATTGGTCCT
TACATTCTTCATTAACTCTAATGCACCTGATTCAGGATCTTGGGGTTTTTCTTTGTTTTTACTCTTTCTTCTCAGTCCCCTCTCCCAATGTCCT
TACAGCCTCTGCCTCAGGGAC
HUMAN SEQUENCE - mRNA
CCCCTGGGCCCTGCTCCCTGCCCTCCTGGGCAGCCAGGGCAGCCAGGACGGCACCAAGGGAGCTGCCCCATGGACAGGGCCCCACAGAGACAGC
ACCGAGCCTCACGGGAGCTGCTGGCTGCAAAGAAGACCCACACCTCACAAATTGAAGTGATCCCTTGCAAAATCTGTGGGGACAAGTCGTCTGG
GATCCACTACGGGGTTATCACCTGTCAGGGGTGCAAGGGCTTCTTCCGCCGGAGCCAGCGCTGTAACGCGGCCTACTCCTGCACCCGTCAGCAG
AACTGCCCCATCGACCGCACCAGCCGAAACCGATGCCAGCACTGCCGCCTCCAGAAATGCCTGGCGCTGGGGATGTCCCGAGATGCTGTCAAGT
TCGGCCGCATGTCCAAGAAGCAGAGGGACAGCCTGCATGCAGAAGTGCAGAAACAGCTGCAGCAGCGGCAACAGCAGCAACAGGAACCAGTGGT
CAAGACCCCTCCAGCAGGGGCCCAAGGAGCAGATACCCTCACCTACACCTTGGGGCTCCCAGACGGGCAGCTGCCCCTGGGCTCCTCGCCTGAC
CTGCCTGAGGCTTCTGCCTGTCCCCCTGGCCTCCTGAAAGCCTCAGGCTCTGGGCCCTCATATTCCAACAACTTGGCCAAGGCAGGGCTCAATG
GGGCCTCATGCCACCTTGAATACAGCCCTGAGCGGGGCAAGGCTGAGGGCAGAGAGAGCTTCTATAGCACAGGCAGCCAGCTGACCCCTGACCG
ATGTGGACTTCGTTTTGAGGAACACAGGCATCCTGGGCTTGGGGAACTGGGACAGGGCCCAGACAGCTACGGCAGCCCCAGTTTCCGCAGCACA
CCGGAGGCACCCTATGCCTCCCTGACAGAGATAGAGCACCTGGTGCAGAGCGTCTGCAAGTCCTACAGGGAGACATGCCAGCTGCGGCTGGAGG
ACCTGCTGCGGCAGCGCTCCAACATCTTCTCCCGGGAGGAAGTGACTGGCTACCAGAGGAAGTCCATGTGGGAGATGTGGGAACGGTGTGCCCA
CCACCTCACCGAGGCCATTCAGTACGTGGTGGAGTTCGCCAAGAGGCTCTCAGGCTTTATGGAGCTCTGCCAGAATGACCAGATTGTGCTTCTC
AAAGCAGGAGCAATGGAAGTGGTGCTGGTTAGGATGTGCCGGGCCTACAATGCTGACAACCGCACGGTCTTTTTTGAAGGCAAATACGGTGGCA
TGGAGCTGTTCCGAGCCTTGGGCTGCAGCGAGCTCATCAGCTCCATCTTTGACTTCTCCCACTCCCTAAGTGCCTTGCACTTTTCCGAGGATGA
GATTGCCCTCTACACAGCCCTTGTTCTCATCAATGCCCATCGGCCAGGGCTCCAAGAGAAAAGGAAAGTAGAACAGCTGCAGTACAATCTGGAG
CTGGCCTTTCATCATCATCTCTGCAAGACTCATCGCCAAAGCATCCTGGCAAAGCTGCCACCCAAGGGGAAGCTTCGGAGCCTGTGTAGCCAGC
ATGTGGAAAGGCTGCAGATCTTCCAGCACCTCCACCCCATCGTGGTCCAAGCCGCTTTCCCTCCACTCTACAAGGAGCTCTTCAGCACTGAAAC
CGAGTCACCTGTGGGCTGTCCAAGTGACCTGGAAGAGGGACTCCTTGCCTCTCCCTATGGCCTGCTGGCCACCTCCCTGGACCCCGTTCCACCC
TCACCCTTTTCCTTTCCCATGAACCCTGGAGGGTGGTCCCCACCAGCTCTTTGGAAGTGAGCAGATGCTGCGGCTGGCTTTCTGTCAGCAGGCC
GGCCTGGCAGTGGGACAATCGCCAGAGGGTGGG
HUMAN SEQUENCE - CODING
ATGGACAGGGCCCCACAGAGACAGCACCGAGCCTCACGGGAGCTGCTGGCTGCAAAGAAGACCCACACCTCACAAATTGAAGTGATCCCTTGCA
AAATCTGTGGGGACAAGTCGTCTGGGATCCACTACGGGGTTATCACCTGTGAGGGGTGCAAGGGCTTCTTCCGCCGGAGCCAGCGCTGTAACGC
GGCCTACTCCTGCACCCGTCAGCAGAACTGCCCCATCGACCGCACCAGCCGAAACCGATGCCAGCACTGCCGCCTGCAGAAATGCCTGGCGCTG
GGGATGTCCCGAGATGCTGTCAAGTTCGGCCGCATGTCCAAGAAGCAGAGGGACAGCCTGCATGCAGAAGTGCAGAAACAGCTGCAGCAGCGGC
AACAGCAGCAACAGGAACCAGTGGTCAAGACCCCTCCAGCAGGGGCCCAAGGAGCAGATACCCTCACCTACACCTTGGGGCTCCCAGACGGGCA
GCTGCCCCTGGGCTCCTCGCCTGACCTGCCTGAGGCTTCTGCCTGTCCCCCTGGCCTCCTGAAAGCCTCAGGCTCTGGGCCCTCATATTCCAAC
AACTTGGCCAAGGCAGGGCTCAATGGGGCCTCATGCCACCTTGAATACAGCCCTGAGCGGGGCAAGGCTGAGGGCAGAGAGAGCTTCTATAGCA
CAGGCAGCCAGCTGACCCCTGACCGATGTGGACTTCGTTTTGAGGAACACAGGCATCCTGGGCTTGGGGAACTGGGACAGGGCCCAGACAGCTA
CGGCAGCCCCAGTTTCCGCAGCACACCGGAGGCACCCTATGCCTCCCTGACAGAGATAGAGCACCTGGTGCAGAGCGTCTGCAAGTCCTACAGG
GAGACATGCCAGCTGCGGCTGGAGGACCTGCTGCGGCAGCGCTCCAACATCTTCTCCCGGGAGGAAGTGACTGGCTACCAGAGGAAGTCCATGT
GGGAGATGTGGGAACGGTGTGCCCACCACCTCACCGAGGCCATTCAGTACGTGGTGGAGTTCGCCAAGAGGCTCTCAGGCTTTATGGAGCTCTG
CCAGAATGACCAGATTGTGCTTCTCAAAGCAGGAGCAATGGAAGTGGTGCTGGTTAGGATGTGCCGGGCCTACAATGCTGACAACCGCACGGTC
TTTTTTGAAGGCAAATACGGTGGCATGGAGCTGTTCCGAGCCTTGGGCTGCAGCGAGCTCATCAGCTCCATCTTTGACTTCTCCCACTCCCTAA
GTGCCTTGCACTTTTCCGAGGATGAGATTGCCCTCTACACAGCCCTTGTTCTCATCAATGCCCATCGGCCAGGGCTCCAAGAGAAAAGGAAAGT
AGAACAGCTGCAGTACAATCTGGAGCTGGCCTTTCATCATCATCTCTGCAAGACTCATCGCCAAAGCATCCTGGCAAAGCTGCCACCCAAGGGG
AAGCTTCGGAGCCTGTGTAGCCAGCATGTGGAAAGGCTGCAGATCTTCCAGCACCTCCACCCCATCGTGGTCCAAGCCGCTTTCCCTCCACTCT
ACAAGGAGCTCTTCAGCACTGAAACCGAGTCACCTGTGGGCTGTCCAAGTGACCTGGAAGAGGGACTCCTTGCCTCTCCCTATGGCCTGCTGGC
CACCTCCCTGGACCCCGTTCCACCCTCACCCTTTTCCTTTCCCATGAACCCTGGAGGGTGGTCCCCACCAGCTCTTTGGAAGTGA

TABLE 2

MOUSE NOMENCLATURE
ICSGNM N/A
Celera mCG15938
HUMAN NOMENCLATURE
HGNC BAT1
Celera hCG1641022
MOUSE SEGUENCE - GENOMIC
TGTGGGCAGAAGGCCGTCCGTCTCTTAACAGACGGGCCTCTCCTCCAGTTCTAGTCTGGAAGCTGCTCTCCAGGGAACTGCTTCTGCTGTCACT
GCAAAGGCAACACCTACGCTGTTTCCAGAACTGTGGCATTTGGCAAAACTGATCTAGAAGTATGCCATCTGGGGCTAGGTCAAGAGGCTGGGGG
AGCAGGGGCAGGAGAGTGCAGGGTTCTGGTAGGAAACCCCTGAGTGTTAACCTCAGCGGGATAAAACTAGTCACACCATAGCCGCCATGTAGGT
TAGGTGACATCTGCTTGATGGCTTTTTTTTCCTTCACTTTTTGAGACTCTTAGGCCCTGCCCAGACTGGCCTTGAGGTGTTCCCGAGGGCTGGG
ATTACAGGAGTGTGTTGTGAACTTCCTGTCTTTCTAGGCGGGGAGGCTGTTTCTGGGCTGGCTATCTGCCAGTAAGTAACACAAATGAACTAGA
AAGAGGCTGTCTGCACTGTGGAGCCACTACCGATGCTGACTGGAGATGTTTTGTTGACGTCTCTCTACCGTGGCTCCGGCTAGCTTGGAACTCA
CTATGTAGAGCAGACTGGCCTTCAGTTTGCAGAGCCCGGACTGCCTCCGCCTCCAGACCGCACCCACCAGCTTAAGTAGGATTTTTGTTACAGA
ATTTTGGTGAGAATGAAGAAAACTGGTCTGGCGCCTGGTAAGTGTCAAGGGACATGAGTCCATCCCTAACACACAGCAGCAGATCTCCGGCATA
ACACCAAGTAAAAGTTTCAAGTCCTCTGGCCGGCCCAGCTGATCATGTGACAAAGCATGGATACTTTGTAAGGTGTTTGTGGCATTATTACAAG
GAGAGAAGCAAAACTAAGGGGGTGGGGGTGGGGTGAGTGGAGCTGGGATCTGACCTGAGCAGTCAACTGCAAGGTAAAAAACCATCAAAGAAAC
AAAGTAGTCACTGAGCTGAAAGCTAGGAAATGTAAAGAAAGAGTTGCGAGCTATGCGGCTGGGTGTGGGAGACACAAGCCTACAACCTCAGCAC
TTGGGATGCAGAGGCCAGCCTGAGAGACTCGGTAAGACCCTGACTCAAAAACAAAAACAATCGGGCTGGTGAGATGGCTCAGTGGGTAAGAGCA
CTCACTGCCCTTCTGAAGGTCCTGAGTTCAAATCCCAGCAACCACATGGTGGCTCACAACCATCTGTCTTGAGATCTGACGTCCACTTCTGGCA
CGCCTGAAGTCAGCTACAGTGTACTTATTTATAATAATAAATATGGGCAGGAGAGAGTGCGGTTGCCCTAAAATTCAATTCCGAACAACCAGAT
GAAGACTCACAACTATCCGTACCGCTATAGTGTGTACTCATATACATAAAATAAATAAATCTTCAAACAAACAATCACACCACGCTAGCACACA
CCCCTTAGTTGATTGGGCGATGTTAGGATAAGCTTTTCTGTTACTAGAGAGGTATGGCCACATCCCAACCAGGGATCTTCAACAGTTCCCAAGG
ACCAAAGAACTACCTTATTTCCCAACTATTTTGTTGTGTGATACAGGGTCTCTCTGTAGTCTTGGAGCTCACTGTGTAGGCTACATACTTAGAG
AGATTCTCCTGCCTCTGCTTGCCCAGAGTCAAAAGACCTGCGCCACCACACCAGGCCTTTATTTCCCACGCATTCTTTCTAGTTCAGACCTGGC
CAGCTTCTCAAGACCAGTTCCCACGGACCCACTCACCATCAGGGCCCTGGCGGGCAGCAGCGTGCAGCGCCGTGTCGCCATGGCGGTCCTGGTG
GGCAGGGTCAGCGCCGAGACGCAGCAGCAGGCACAGGGCAGGGGCATCGTGGCGGGCGCAGGCCCTGTGGAGAGGCGGGGGCTGCCCAGCGTCC
ACATCAAGCCCGGGATGTCGCTGGAGGAGAGCCTGGGCTCGCACCAGCCGTCCTGCAGACAAGTACCGCCGGAAGCGTCGCTCTCGGCGGTGCC
GGCGGGAAGCGGAGGCCATGGAACAACTCTGGGGCTGAGGAAGAAGTGGGAAATGGCAGCGGTGAGGTCCCAGCTCCGCTCTTCCCCGTTTCTC
CCTTTACCTCCCATTTCCTCCCCCTTGCATCTCCAGCAACAGATAAGGCCTATTCTAAACTCTCATCCCTTAGCAGATGGCAAGTTTGGGGCGC
AGGGTGGGGATGGGGTTGGAGTTGGGGGCTGGTGGGGAGGGGGGAATCACAGAGTTAATTTTTGATGTAATTTTTGAGAAAATGATACATAGTT
GTTTTAAGGTTCCAGAGCCCAAGAAAAAAGTTAATTCTGAACTTTGGAGGGAAACAACAACAACAACAAAAATCAAGTAAGGCCAGATGGGGGC
AATTTTCATTAGCCACAAATCTGGAGAAAAGTCACAAATTCAAAGAGCGATCTAGAAATGGAATATAATGTACCCGACAGACAGACGTAGAGGC
TTCTTCCTCTGGGGCCTGGGGGGAGGGGTTACTCATCAGACCTGCTCCCCGCCCCCCCCAAGGACCCTCAGAGACGGAGGCCGGAGGCCTGGGC
TTAAGACGCTCGGAGAAGGGAGGTGGGGAGGGAGGAGGCGCGCCAGGATGCAGGAAATGGCGCAAGCACACGCGCAAGTTTCGGGCGGAAATGA
ACTGTGTGTTCCATCCCGGAAATCGTACCGAACGCTCGGACTGGGTGACTTGTGATTAGGTCTCCGGAGGGGAAACAAGACAATTTGCGACATA
GGGTCCTCCTGCAAGCGGAAGATGAGAGGCGAGCTTTCGAAGGGAGGCAGAGGCAGAGGCAGGCCTGGTCTATAGGGCGAGCTGCAGCGACAGC
CAGGACTACAGAGAGAATCCCTGTCTCGAAACACACACACACAATCTCACACACACACACATAATCACACACACACCACACAACCACAATGAGG
CCAAATATATGGGTAGATCAAAAAAGAAAAACACATGGTTAGACACTAACAAGGCAAACGTAGGACAGAGCAGGGAGATGTGTGCAGACGTTCG
GATTTGGGGGGAAAGTCTGAAGAGAAGATGGAGGCTGAAAAGAGGGGGGAAGGGCGGAGGGAGGCGTGGAGACACAGTTGGGAGTGTAAGTGAA
ACCCAGCGTACTGTGTCCAAGCGCATCCTTACCTTCCCACACCATTCTCATCGCCTTTGTGTCTCCTTCGAGACCACCAATCAAGATCTCACAC
CCAAACACTCTGCCCTCCGCTTCCCCCAGACCCATCTCTGTGGACCCTGGTGACACGGAGAGGCCCTGCACCTGCTGCAGTAGTCAGCACAGGG
CTGGTCTCTGCTGCCCTCGGGCGGCCGTTTCGGGTACTACCTCAGTGAGGTTCGAGAGGAGGACCAGTCATCAGTGTGAGGATGACAGACACCC
TCCCGGTGCAGGAGACTGAGTGAAAACGGGAGCGCTAAGACCCAGTGGGAGTGGAGGACCGCATCAAGGGCCCGGAGGAGTAGGCGCTGGAGGG
TGGGAGCCGTCTGGTACCTCGGTCGCCTTCGGAACAGCATCCTCTCTAGTGATAAAAATGGCCAGTCAGACCCAGGGTATCCAGCAGCTCCTCC
AGGCTGAGAAGCGGGCAGCGGAGAAGGTGGCCGATGCCAGGAAGAGTGAGCCCCCCTTTCCTCTCTTAGGAATATGGAAGGAGAATTTAGGAAT
AGAGGATAGCGGGCATTTGAGTCCTTAGGAAAAAAAAGCCCAAGGACAATGAGGGCAGATGGGGGACTGAGGCTATTAGGGTAGAAGAAACGGG
TGGGTTTTACAGTCTAGCATTTGGTTGGTTGACAGGAAACTGGATAACTTTCTAGAGGGCACTGATCCCATTAACGCACCTGGTTGTGTGTGTG
TGTGTACACTCACTGTCCTTCTGCCTCTAGGGAAGGCCCGGCGACTGAAGCAGGCGAAGGAGGAGGCTCAAATGGAGGTGGAGCAATACCGCAG
GGAGCGGGAGCAGGAGTTTCAGAGCAAGCAGCAGGCGGTGAGTTGAGGGGGACAGGGATGGCCCCACCCAGGTGCAAATCGGTGGGTGCCTCTT
GCCAGGTGTATACAGGTGACTAAGAAATTAGGGTGTCTGAGAGCTGAAGAGGCTCTGACAAGGACCACACATTGGTAAAGCTTTGACGGGATCT
GTAAGGTCTGAGGGTTTTAAAGGGTACATGAGACTTGTGGGGCGGCCTTCACAGTCTGTATGGTGGGGACAGTTTTATGGTCAGTCTCAGAAGT
AAGGATTGTGCCCACCTGCTTGGTATCTACATGTTTTTGTGGGGTGGGAATAGTTTCGAAAAGGCTTCCCAGGCCTTCCTCTGAACCCTTCCTC
CTACATGCTGGGACCCTCCTTTCTCTGTGTCTGCTTTTTTCTTTCTCTCCTCCGCCCCCATCCCCCAGGCCATGGGCTCTCAGGGGAACCTGTC
TGCTGAAGTGGAGCAGGCCACAAGACGGCAGGTTCAGGGCATGCAGAGTTCCCAGCAGAGGAATCGGGAGCGCGTCCTGGCTCAGCTTCTCGGC
ATGGTCTGTGAAGTCAGGCCCCAGGTCCACCCCAACTATCGGGTTACTGTCTAGAACCATCGCTCAGGGACACATCCCTAGAGTGACTCCTTCT
GTCAGCTCCGTCCACAGAGAAATATCCCAACTCAAAACCACTTGTGTCGCATGCGCAGAGCCTTGGGTTCAATCCATAATATCATCCCCCCCAC
CCACCAAATCGCTTCACATAATAACCTGTTGCTGGAGGGGAGGCTTCTATGTGACAGGATCCAATATTCCCCCCTGAGACTTACAAGTAGCCTG
TTCAAACCCGACAAACCCGAATCTCTCTGTTCCATGACACCCACTGGAAGTTTACTAATGAATGCCCACGTCATCCCCACCGGACCCCTCTGTG
AAATATATGTCCTGGTGACAGTTATGAAAGCACCCTGACTTCAGAGCAGGGGAACCTCTGTTCCTCTCCCACCCCTGGTCCTTGCTGGTTACAC
AACTGGGACAACTATTTAACCTGGAAGCCTCAGTGTCCTCATCTTTGTGACTGGAATGACAGCAACAACGCTCACTTTGTAAGGTGGCTGCTGT
GAATTATCTGTCAGAATTCAGCATAGTTCCAGATGACGGAGTGCCTGTATCATGACCGCACTCTGCGTGCCTGCCAGAATTCTAACCCCCTCTC
CTGACTTTTTTATTCCTCGTGTTCCAGCTTTGTTCAGCCACTGTGACACTAGTAACCCTCTAAAGAATTTGTTTCTCTGGCTCTCCCTTGGCAC
ATCATGCCCCCGAACAGTTAAATGATAATGTTATTTAGCCCCTCTGTCCGAGTCCCAAATATATATCAAATCTCTTAGCACCTCTCTGTTTCTC
CTGAGCTGGTCTTTCTCATGGTTGGCCCCCCTCTTTCTCTGTTTTCCCAAATCTTGCTGCTTCCGGGAGCTGAAGATATCTATGACATTCACAT
GCATGCATTCCTTTGTGTGCCCTCAGCACCTTGCCTCCTTGTGTAACAGCGGAGTGTCAATAAAATACTGGTGGATGAGTCAATTGTCGGTGAA
AGCAGCCTCCTTTTTGCCTTGCCGTTGCTCACTTTGGTTCGCTGTCCTCAGAGTTATGCATTTATATAGTTTCCAAAACACTGCAAAGGGCTGT
CCTGGAAGCTTTATGGAGAAACAAAAGGACACAGTACTGTGTCCCCTCTTGAACAGAGAGTTGCATAGATAGTTGTAAGGGAAATGTTGGCAGA
GCAGCACTCTGTCCCTTTCCTGGTTTTACACTGCTCATCAGCACCAGAGACAGAGACAGACAGACAGGCAGGCAGGCAGACAGACAGACAGACA
GACAGACACACACACACACACACACGAGAGAGAGAGAGAGAGAGAGAGAGAGACAGAGAGACAGAGAGACAGAGAGACAGAGAGACAGAGACTT
ACTATCTTACTAAGTATCTCTGACTACCCTGGAATTTGCTAGGCCAGGCTAGCTTTGAACTCACAAGAGATCCACATGCCTCTGCCTCTTGAGT
ACTGGAATTAAAGACGTGCACCACCACGCCTGGCTTGACATTATGTTTGGGTATTGGCTCTCATTATGTATCTGATGATAAAGGTCGTTGTATC
CCCAGAGCCTAGATAGCACTTGGCACACACTTGGCAGTTGAGGAATACTTTGGAATAAAAAGATGCTGGGGTGGGGAAGGAGGAAGTTGGGGTG
GTGGTGGTAGGGGGTGAGGGGGTGGGACACTACTGGTCTCTGACCCCCATTCCTTTATCAGTTCTCTTCACTGCTCATTTCACCCTGAACTTGA
ATCAATGAGGTAATTCATAGTCTTATATGACTTTTCTGTAGAGAGACATTAAAAAAAAATCTATAAGCCAGGTGTGGTGGCGCATGCCTTTAAT
CCCAGCACTTGGGAGGCAGAGGCAGGTGGATTTCTGAGTTCAAGGCCAGCCTGGTCTACAGAGTGAATTCCAGGACAGCCAGGGCTACACAGAG
AAGCCCTGTCTCAGGAAAAAAAAAAAATCTATGCCAAATAAGGCAGTCATGAAACACCAAAGATATCTCACCACCCAAGTCTCACTTGGCAGAC
CAAAGAGCTCGCTGGAGTTATTGGCAGTATGGTGGGTGATAGGTTACTTATAGGAGTGAGACTGGAGTAGCTTCCCATCAGAAAGTCGCCCACC
AGCGTGACTTGTATGCAGACCAAAGAGTCCTGCCACCAGGAGCTGTTTATTGGTGACACAATCTGGGAGAAGTGTTCCGTGTCTTGCAACTTTT
TTTCTGAATTTCCTGAGCCTTCTCCACTCTCATCAGGAGGAAATGTTTCGGGGGAAATAGCTATACAAAAGCAAGGAGATTTTTGAAGAGGAGG
GACACATGTGATAGGAAATTGGGGTGCTTACACCCTGGTGTTTCCCTGCTAGCTGTATTGCCTTGGCCACCTTGTTTTATTTCTCTATTTTCAT
ATGTTTACAGGAAGAGTGGAGCGGGGCATGGGTCTGCATTCAAATCCTCCAGCAGGAGAATTGAGAGCTTGAGGCCAGTCTATATAACTGCTCA
AACTCAGTAACAGAGAAAGGAAGAAAAAAATGGCAAGACAGATGAGGGGCACTCACCTTGCTGTGGCCCCAGTGGCCCAGGTTTTAAGCATTGT
TAAAATCACAGCCAGCTGTTAAGACAATCCCTTCCTGCCCTGGCTACCTTAACAAGGACTGTCAGCATGGCCATTTTGAATCTTCTATAAAGTC
TTTGAGGAAACCACACAGCTAATGGCTCAGCCAGGATTGTTTCTGTCCTCTCTCTCCTCCCCCAGTGGAGGGTGGAACTTCCCATAGTCCAGTA
GCAGGAAGATGTGTTATCACTGATGCCAAAGGGACAATATTATGATTGGTGCAAACACAAGTCTTCATTTGCACGAGTTTGCTGTTTGCTGTGT
TTGGTGCAAACACCCTTTGCTGGCTGTAAAGCAGAAAGTTTGGCTCACTTGGCTCAGCTGGGACAAACCTTACTATGCTACCCTCCACAATGTC
ACCACATTAGGTCCAGTAACAGGAAGACAGTGGCTAAGAGCCCGCCAACTAGGTCACTAGGAGAGTTCCTGGAAACGCTTCAAAGCTAAAGGCC
CAGGTACCGCACTTTCTGGAGCTACAGTTCGTAATAAGTTACCAGAGCCGAGGAATTCCCACTCCTCTCTGATTTATACAAAACCCGCCGGCCT
GCTGCTTAGGGCTGCCAAATGCGGAGGGATCAAAAGCTACCAAGCCCCAGCCCAGAGAGCTTATGCGATGAGCAGGACACAGCCAGCTGGTTGG
CAACACTGATACTTAGTGTTACTTTGTATTAATATTAAGTGTCCTTAATAGGGAAAGCCCGAGCTTGTGTTTATGTAGCAAGGCAACAGGACAA
CACTTCTGCTATATGTAACATCTCAAAGGGAATACCACATGGGGGGAGGGGGATGTCCAGGCTGAATAATGGAACTGAGATTATTCTGCTGCTC
TAGTTAGCAAGAATTATGGAAAGGTGCATAACCTCAAACCCCACCATTTATTTAGCACCTAGACAGAATGTAAGCCTCCATCTTTCGATATAAT
TTTGGGTAAATCCTGCCTATTCTCTGCACGACTGTAATGGGCGTGGTCACGTGTCCCCCTCCCCTCCAGCAGAGGCCTGAGTTAGCCGCTCTCG
GGTCACCTTGACTACGAGGCTAAGGACCCCGTGAGAAACGCTTCTCATTCGATCGCGGAGTCCTCCATGCCCACAGAGAGGTGCCCAGGGAGAA
GCCTGGCGTGGCAAACAAAACTAAAGTAGAGCCGACCGTCGGAGGGTGTTGCATAAGCGTAGAAGAACAAAATGGAGAGCTCTGCGGGGGAAGA
CGTGAGTTTCCTGTCGCGCTCTTGCTACTGGCGACCGGAGGAGGCGTCGTCAGAAGCTTCATTTCAAGTGGGCGTTCTGCAAAGCCAGCCGCGG
AGCGCGCGCGGCGAAAGCCTGCTTCCGGCTCCTTGCGCGTGCGCCCTGGCGGCCGGGAAGGCGGGAGGCCGGGGCGAGCCTGGAACCGGAAGTG
AAGGCAGCTTCCCGCCTCCGTCCCCGTTGCTGCCGCCATACACGCTCGCAGTGCTTAGGTAAGCTTTGGCCCTGTGCACCATCCACCGCCATCT
GCTTCTCCCGCGGCTCGCCCCGCCGCGCGGGTCCCTGATACCCGGTGCCGGGTCGGCGCCGTGGCCGCCGGCGCGCAGGCGGGCGTCCAGTTCT
GTTGTCTTCACCCCTTTCTGAGAGCAGATCTTGGCGGGGAGCTGGGAGACAAGGGGGGAACGATCCAAAATGGTGGCCTCGGCCGCCATTGTGT
CTCCGCGCTTCCTGGGTGTTCCAGTGGGTCCTCTGCCCACCCGGGGCCGCGGCGGCCGGAGGCGGCGCCAGGAGGAGGGCGGGGCCCCTCGCAT
CTCCCCTCCGGGCCTTTTCTTGCTCGCCCAGGGATGGGAAGATCTCGCTCCGGGGCCCGTCCAGCCCTTTGACCCCGCTTTCGCTGCCTTATTT
GGAGCTCTCTGTGAATGGCTGCCTCTGTGCGTGGCCGATGGTGTCGCGCACTTCTGGGGAAGCTTTTCCGAGAGCACTGCAAGGCGTTCCTTTC
TAAGGCCACCCAGCTCACACTCTTAATCCTGCATCGTTCTCAGCTGTGACCTTAATTCCTTAGTGCGACTTTTTAAATTAACTTTGGCAGCGCT
GAGCCGAACCTTGCGGCCTCTCGCGTTACTAAGCAAGCACTGCCATTGAACTGCACACACCCATCTTTTTCAAAACAAAAAAAAAATTTTTTTT
TCTTGAAATGGAGCCATTCATACACTTCCCACTCTCCGCCCCGCACCACCCTCCTTCCTGCTTCTGTTCCCCGGTCCAGGTGTGCGCCGCTGGG
ACGGCCACTCCGGGGTTTCTGAGCAACAGAAATAGCGGCAGCCACATGATGTTTGATTTGGGAAGTGAGCGCTCTGCGCAGTGCTHACCCTTAT
CTATCACCCTTGACTGATGGCTGACGTTGGGGATCACCACCGTGAGGTGGCAGGAGAAAGCGCAGTCTCTGTCTTCCCTTGTCCTTTGTGTCTC
AACCCTCGTGTGAGTCGTTAGTCACGCTTATTTTACTGCGGTCTCCCAGTTGGCTCCTGCCTGTCGAAGTGTGTGTTACAAAGTCTGGTTAGCG
GTGGAGCCCACTCTGTCTCTCCCGTCTGGTGTTCCCGTTTCTTATCTCATGAGACTCTTTGTTAACTCTTCTTTCGGGACAAAGGTTTCACAGT
TTAGGGAAGAATAGGACGGGTGTAGGGATCGACGAGAAGTGACGAGTCCATTCGTGACTGATGAGTTTTTCCGGTTTTTTTGTTGTCCCCTCTC
CCAGCTCTTCTGTCGGAAACTGGTGTCTTTCCCCTTGCTGTTCTTCAACCCCTCTCTTTGGCCCTTGCTTCCTCACCTGCTCTGGGACACCTAA
CTCAGAGACCTCCCTTCTCCCCCTGCCGGCCCAATTATGGCAGAGAACGATGTGGACAATGAGCTCTTGGACTACGAAGACGACGAGGTGGAGA
CAGCCGCTGGGGCAGATGGGACCGAGGCTCCCGCCAAGAAAGACGTCAAGGGCTCCTACGTCTCCATCCATAGCTCCGGCTTCCGAGATTTTCT
ACTCAAGCCAGAGCTGCTCCGGGCCATCGTTGACTGTGGCTTTGAGCATCCATCAGAGGGTACATTTTATTGTTGTGTGTAGAGACCTTATTTA
GCACCTCTGGTGCAAGAAAAGGGAGGTTCAAGTCTAGGGTCATAGGATTTGAATTTATTTGGGATAGGCCAAGCTCTGAGAATGTACCCAAGAC
GGCAGCTGTAGAAGAGGCTTTTGTTCTCCATTAAACCGAGGGCTGCCATTTGTTCTGTGCCTGGCTTTTTTGCTTGGTTTTTGTCATTCTGACT
GTTCGAACTCCCAGAGAGGGCCTGGTTGGACCTTTAGTTCCCTTCTTTTGGGCCAGGCCAAGTGTCGTTTCCGGAAACCTTAACGATCAGGATG
CTGGCTTCTGTGGCTTCTGGGTCAGGTACCAAGTCCTTCATTTTGTTCAAGGTTGCAGGGTTACATGACCCAAAAGCACAGCAACCTATGTAGG
AGAAATGGCCTGTGGCAGTCGAATTCATAGATTGTTACGGTGTAAGAAAAGTAAGTTTAGCCAGGAACGTGGAAGGTGGCCTGTTGGTAGCTTC
TCGAGGGTTCTTTCTTAGCGGAACTGGGGAGCTGGGGTAGGAGGGTTTGACTTGAGTGTAAGAGTGATGAGGGTCAGGGAAAGGATGGGGTCTG
AAAGGTGACAAGGTGACATATGATGAGTCGGCTGGGGAGAAAGGGGTTTGGCATGGTGCAAAAATGTCTCTCCTTTCTCCTTCTAGTCCAGCAT
GAGTCCATCCCGCAGGCCATTCTGGGGATGGATGTCCTGTGCCAGGCCAAGTCAGGCATGGGAAAAACAGCAGTGTTTGTCCTGGCCACACTGC
AGCAGCTGGAGCCCGTTACTGGGCAGGTATGTTGGGGGCAGTGCTGGAGAGGGTGTGGAGATTGAATCACCAGGAGGCCATTTCTGGCTCCATG
TGCTCTCAGCTGGTGTCCTCTGCAGTTCAAGGGGAATGTTCACCAACATCACTGACAAGTTGCTGCATGCTCTTGTGACGAGGGGCTGACTTTA
CTGTGTCTGTCTCCATTTGCTCCCTCAAAGGTGTCTGTGCTGGTGATGTGTCACACTAGGGAGCTGGCTTTTCAGATCAGCAAGGAATATGAGC
GCTTCTCTAAGTACATGCCGAATGTCAAGGTAAGGGGGGAAAGAAACCTGGGACAGGAGGCTGTGGGGAGACAGGCACTGGGAGGGAGGTTGGC
TGTCCTCGGGCATCCCTGTGCTGTCAGTGGTGTGGTCACATGAGAGACTTCAGTTAGTGCCACACCCTTCTGTGGCTGCCCTTCCAGGTTCCAT
ACTGTGAATACACCTGTGTGTTCCTGAGAGTTCGTAGCTTTAGGTGATACCACCGCAACACTTTGTGGTTAGTTGGTTTTGGTATTGCAGATTC
CATTCCAGAGCCTTGAGCTACTATGTGTGGGACTGAGCCACCCCTGGGTCCTTTGTTTTTATGTTCACGACTGTCTCTTAACCACCACGTTGTC
CTGGTAGGCCTTGAACTCACCTTAGTCTAGAGATAGTAGGCCTTGAACCTGAGGCTCCTGCCACAGGTTCCTGAGTAGTTGGGGCAGGCCTTTG
CCACCTGGCTTGGCCAGAGCCTCTGTTCTGTTCTCCTGAATTGCTTGGTAGATTGTTTGAGAGTTGGGTTGTATGTGGCAAGAGGAAGCTAGAT
GCTCTTAAAACAGCCCTGGTGTGGACTTTAACTCTAGAGTGGTTAAAGCTTTCCGGGGGTCATAACAAGTGGCCTGTGTCCTCTGTGAAGGAAC
GTGGTGTGGCCTCACAGTTGGCATCCTTTGGAACTTTTGAGATTATATATTTCCATGCTTGGGTGAGTGGCCTGCTGGGTTTGAAAGGCCTTGT
AAGCTGGCCCTGTGAGTAGAGAGATGTGTGCAGACTTCCTATTCTGGTTAGGGTGGGGAAAGAGCAGCTTGGATCAGTCTGGGCTGACCTTTGA
GTCAAATCCACCTTCCTCTGCCTCTACTGGGATTAAAGGCGAGCATCTCCACAACCGCCCAGCATGACAGATTTAAAAGGGGGAATTCTAGAAG
AAGAAAAGGAAAGGCAGTATGTGCAAAATGCTAAAGGAGGCCAGAGATCTTGTGATCTCTGAGGTTTCCATTGCTCGGCATAACCAAGCATTAG
ACATTGTTTTCCTAACCTGTCTGTGTGCAAGCTTACTGGCCTCTGAAACATTTTAGCGTATTTATATATCTTTTTCCTAGGATTTTGAAAATAG
ATTGTAATCTGGAGGCATTGTCTATGTCTGTGTGCCTGTGCCTCTGGCTGTATGTCATTATACCTTGCCAAAAAACTAATAGACTTTCAGTTCC
TTGGCTGACCCTTCCCCTTGTGGTGGATTTAATAGTTGGGAACTGAAGCTGGATCAGTGGCTCATTCCTGTATTCCCAAGGCCTGGGAGGTTGA
GGTGGGCGTTTGCTGGAGTTGAAGGCTGCCCGGGGCTAACGTCTCAAAACAAAGAGGGAGGGTGGCAAAGCTGCAGTCCTTCTCTGCAGGTCCC
ACAGCACCCTTGTGCTACTCAAAATGCTCTGAGAGTTGGGATCAAGTCTGAGGTTGGGTTTGGTGTAGGAGCCTGCAGATTAGCTGGTTAGGTC
CCCCCCCCCCATCATAAAATCAGAATCATCTGTACTTCTCCTTGGCTGTGCAGCACAGTGTCCTCTAGGGGGCTGTCTAGTCCCTCCCTTAGTT
TTAGCTCGCTTTTCCCTCAGTATTTGTAACTATGTCTGAAAGCTTAACTGATACCAGTAAACCATCGTGAACAGGAGTGGAGTGCTCCTGTGTG
CATGTGTTAGCCATTGTGGTTCAGGATTGAGAAGAGGAAGAGGTTTACTGTAGAGTAAGCGTCCTGGTTATTTCTTTCCCCTTGAAATTGGACA
AGTCCACGTGTCACAGCTCACACATCCAGCACTCACCTGGTGTCACAGCTCACACACTCACCTAGTGGGTTGCACATGGTGTGACTTTTATCTC
AGGCACCGAGGAAGCAAAGGCTCCAGGCCAGGGAGACTGTCTGACAGTTCAGTTAGGAAAGCTTGCTTGTGGTTTGATCGTTGGGTGGCGCCTC
ACACTTGCTGGTGGCTTCTTGCTTTCTTCATCTGTGTAAGGAAGCTGTGACTTTGGAGTCACACGTGCCTCGTTGTGTAGATCAGCTAGGGCTG
TGCAGAGCGATCCTGTCTTGGAGTTTGAGTGGAGGCGGAGAAGCTGAGTGTTCATCCCTTCCTCCATCCCTCTTGGCCCCCTGGTGCTGAGGAG
GTCGCTTTGTTTCTTATGGGAGGCACAGGAGTCACTGCAGGCCTGAAAGCTAAGAGTTGGTTTACAGTCAACCACAGAATGGACACACTGGTAT
TGGCAAGATGGCCCACTGCCTTTAATCTGTACTTGGTGCCAGACAGGCTGATCTACAAAGTGAGTTCTAGGCCAACCAGGGCTATACAGTGAGG
CCTCTCAAACCAAAGGGATGGGGGAGAATTCTGGGAGACTGGAACATTGTCCTTTCTCGTGGTTGACAGGTGGCAGTGTTTTTTGGCGGTCTGT
CTATCAAGAAGGACGAAGAGGTGCTGAAGAAGAACTGCCCACACATCGTCGTGGGGACTCCTGGCCGAATTCTAGCCCTGGCTCGAAATAAGAG
CCTGAACCTCAAACACATTAAACACTTTATTTTGGACGAGTGTGACAAGATGCTTGAACAGCTCGGTGAGTGGCGGTGCCCAGGCCGCAGCTCA
GGTGGTTTGGGGAGCAGCCCTTTGAGCCAAATGATGTATGTTTGACATAGGAGCACTTGTGTGCAAGGACGACCCTTATCTATCACCCATGACT
GATGGCTCTGGGCCTCCTTCTCTTCCCCTGGTGCTGTACAGTGATGCGGTGTGCTCAGCCCTGCGCTCCTTTCTGTAGCAGGGAACGGAATGTG
CTGACGCTACAAGTCCTCACCACCACCCTCTCCTTAGGGATGGACCATGTACTGGGTGTTACACAAGAGCAGCAGGTGCCTGCCCTTGTATCCC
AGTATAGCATCCGTGATAAGGATGAAATTTCATTAAGAGTTTCTTTGGGGAGTGCTAGAGAAATGGCTCGTTAGTTAAGAGCACTTGGCTGCTC
TTGCAGAAGACCTCGTTTCATTCCCAGCATCCATATGAAAGCATATAACCACTATACATAGCTCCGGTTTCCAGGGGTGCCTGAAACCCTCCTT
TGGCTTTTTTGAGCACTAGGTGCATTCGTGTTGCTTAGATGTACACGGAATGAATATAAATAATAACATAACGGAGCTCCGGCTGCTAGTTTTG
GCACATGCCTTTAATCCAAGCACTTCAAGAGTCTAAGGCAGGCGAGTCTCTGAGTTGGAGGCCCATCTGGTCTAGAGTCAGTTCAAGGATATCC
AGGACTACACAGAGAAATTCTTGAAAAAAAGCCTTAACCTGCCTTTAATCCCAAGGGCTACATATCGAGCTCTTATCTTTAAAAGAGGTGGGGG
AGAGAATTGATTCTGTTGAGTTGGGAGAGTAGAAAAGAGTGTAGCTCTAGAGAAAAGTCTTTACAAATGGCCTTTGACTGTCTCAAGATGGCAC
TCTGGTGGCATTAGATCGGCTGTATATGTCTCATGGAACAGGGTATGTGAGGCGAGAGGTGAAGGTGGAAGGCACAGTGGACTGGTGTAGAGAT
TAAGTTCTGAGATGGGCGTAGGGAGAGCAGCAGGAAAGGGCCTTCAGCGGTATTTTCTTGGAGACTGTATGTATGTATGTATGTATGTATAGGT
CTTCTTGTGTAGAACTAGTTGGACTGGAAATTATGTTAGAAACCAGGCTGGCCTCAAACTCAGATTTGCTTACCTCTACCTCCTGAGTTCTGGG
ATTACAGATGTAAGTTTTTTTTTTTTTTTTTTTTTTTGAGTCAGGGTTTCTCTGTAGAGCCCTGTCTGTCCTGGAACTCACTTTGTAGACCAGG
CTGGCCTCGAACTCAGAAATCCGCCTGCCCCTGCCTCCCGAGTGCTGGGATCAAAGGCGTGCACCACCATGCCCGGCAAGTTTATTATTTTTAA
GTGTGTATGTGTGCATGCATGCTTGTGCCCATAGAGGACAGAGGAGTGAACCGCCCTCGAGCTAAGTTACAGGCAATCGTGGGAACTGGGAAGC
TAATGTGGATCTCTGTAAGAGCAGCAAGTGCCCGTGGCTGCTGGACCGTTGTCCAGCCTCATACTCCATATTTAAGGGCTTCTGTCCCCTGAGC
CTCTCGTGAACTTTTTCCCCACTTTGTAGATTCGGTCATATTTCTTGTCAAAACAAGAGTGGAACTTGACCCCAAGTCTTTTTCTAAGCACATT
CTTTATTCCATGCTGTTACTAGTATACACAGCTCCTGGGCCTCCTAAGTGCTGGGATTACAGGCATGTGCCACGAAGCCTGGCTCGTTCCTGAG
ATCATGGAACCGCCACATCTCTGCAGTGGCTCCCCTCAGAGTTTCATTGGCCTCACCAGACTTATGTCACAGACTCACTTCTTACTGTGTGTGT
GTGGTTTTTTTCTCCCCTGCAGACATGCGTCGGGATGTCCAGGAAATTTTTCGCATGACCCCCCATGAGAAGCAGGTCATGATGTTCAGTGCTA
CCTGAGCAAAGACTGGTCTTGTGGGTTTGAGGTCTTGTGTCCATGCAGCTCAGAGGCCAGGATGTTCAGAAATTAAGGCCTAGATGTACATGCA
GTCAGGTTGAAGGGCAGAAAAAAAACATTGTGAAAGGATATTTGGGTTGGAGTAAGGGACGGAGGAATTCCTTTAGAAATTCTGCCAGGTCTGG
GAGGTGGTAGCACATGCCTTTAAACCCAGCATTCAGGAGGCAGAGGCTGGCAGCTCTTAAGTCTACAAAGTGCAATGCAGGACGCCAGGACTAC
ACAGAGAGGGGCTGCCTTGAAAAGCAAAAAGCCAAAGTTCTGCCAGGTGTGTTAGCAGACATCTTTAATCCCAAGCAGTGGGCAGAGACTGGTC
GATCTCTGTGAGTACAAGTGGGGCCAGCCTGGTCTCGTGTTTCAGGACGACTAGGGCAAATAGAGAAACCCTGTGTCAAGCTTTCTGCTCTCCA
GAGAATGCACATTGAGTTCATAGCCCTGTCCATGTAACTGGCCCCAGGGCATCAACAAACACTCCTGAAAGTCCACTTGCCAGACACCCACAAA
ACCACACACACAGAAATCTTCAAAACTCCAAAAATCAGATTTCTGGGGCTGGGGAAGTGGCTCAGAGGTCAAGAGCACTTTTGTTCTTCCAGGA
GTTCCCAAGTTCATTTCTCTATGTCAGCTCCCAGTCATGTAGTTACAGTGGCGGAAGAAGATGGTCCAATGTCCTCTGCTGGCCCTCCAGGCAC
CAGGCACACATGCACAAGTGTGTGTAGGCAAACCCCACACACTTGAACTTAAAAAAACAAGACTGATAAGCTGGTGCATACATCATGCCCGTGC
TCCCTCAGGTGGTGATCTTTGTGAAGTCCGTGCAGCGCTGCATCGCCCTGGCCCAGCTTCTAGTGGAACAGAACTTCCCAGCCATTGCTATCCA
TCGTGGAATGCCCCAGGAGGAGAGGTGGGTTGGAGGCGCGTGCGTGCCTGCCCGTCCATCTGGGTCCTGTCCTGTGGAGGAGGCAGTGTGGAGA
GAAGAGAGTCTCAACCCTCTCATTTACTCTCTCACAAAGGCTCTCTCGGTATCAGCAGTTCAAGGATTTTCAGCGGAGGATTCTTGTGGCTACC
AACCTGTTTGGCCGAGGCATGGATATTGAGCGTGTGAACATTGCTTTCAACTATGACATGCCAGAGGACTCGGACACCTACCTGCACAGGGTAA
GCTGCCCGCCCACCCCACTTCCCGTGTGTGCTGAGCACCCCCCCTCTCCTTTGTCTTCCCTGGGAGGCTTGCAGTCTAACCCTTCTCCTTCCAG
GTGGCCAGAGCGGGCCGGTTTGGCACCAAGGGCTTGGCCATCACATTTGTGTCAGATGAGAATGATGCCAAGATCCTGAATGACGTTCAGGACC
GTTTCGAGGTCAACATCAGCGAGCTGCCCGATGAGATTGACATTTCCTCCTACAGTGAGTACCACCCTATGTGTGTGTGTGTGTGTGTGCCTGT
GTGCGCGTTTCCCATTTTGTATCTTACTGTGACTTTTTTTCTCCAGTTGAGCAGACACGGTAGAGGACTCGCGTGGTCAGTCTGCTGTAGAAGA
GGACACGGGTCAGGAGGAGACACTACCGCCCCACCCGACACCGACGCCTCTGCCCACCCTATCTATGCTTCTCTCTGCGTCACCACTCCTAAAC
CTAGTCCTGATTTATCAGAGTTGTTTGTTTGTTTTTGTTTTTTAACAAAACTAAGAATGAAACAACCGTGTCTGTGTTGTCTGTAAGTGCTCTG
TTCATGGCTTGAACCAGGGTCATTCTGAGGGCCGTGAGCCGGGTTGTGGGGCAGCGTCATTGTCTTCTTTCTAAGGTGGCTGTGGACAGGGAGG
CTGGGACACTGCTGGGGCCCGGAGGTAAAGAGAGCAAGCCCCACGTCCTGGTACCTCAGCTCCTTCAGCTGAGTTTCTTGTACCTCCCAGGTAT
CTAAGCGGGGCCTGGTAGGCCATGCCTGAGCGTGTGTGCACAGGCATGCGCGCGCGCACACACACACACACACACACACACACACACACACACA
CACACACACACACACACGCATACTGGCAGCTTAGCTGTGTAAAGAGCCTGGAGTCCCTAGCTGGACTAAGTGTCAACCAGGGCAGGGGCTGGAA
GCCTTGGGAAGCTGTGGAGAGGTCTGGCCTGCCTTCCCTTTCTGTTTTTGGTCTGAGGCTCAGAAGGTCATAGGTGAAGCCCAGGCAGCTTCTT
ACCTCAGCTGTGCATCTGAGGTAGAGCGGGCCTGTTGCAGGATTATCTTGGGGTTCTTGGCAGTGGGGGGGCCGGAGGTGGGAGGTGAAAGTAA
CAGACCTGAGCTGCTGCTCCCTGGAACCGGAACCGCCTCTCTGTAGACCTTGATTGCACTTCAGGGCCAGGCATTGCTAAGGCACCGGCCCATG
GGCCCCCTGCCCGCTCTGACCTGACAGGCTGGAACTTGTGTTGCTGGCTTGAGATCTCAGATCCACACAACTGCAGTTGCACCCTTAGCCTCAG
CACAATTGGGTGTTGTGATGGATGCCATGTGGGTGGGCGTGCCTCAGAAGGGGTGGATGGTATTACTCCAGGTCACTTGGGGGAGACACCCATT
TCTTCAGACATGCCCCAGAGCACAGGCCTCTGCTCTAAGGTTTGAGCAAAAGTCTCATTCATACCTTCTGTGCCCCTGCCTGTACCTCTCTATG
CCTCCCTGGGATAGCAAAGAGGAGGTTGGTCTCTGCGCCGAAGAGCTTCCCCACAGTCAGGGTCTCATCAGGCGGAACTATACATACAGCCAAA
TGCTTCAGTCTCCGTTTCATTCTTTGTTTTTATTTTCAAAATAGTTTATTAAGTTTTGACAGCATCTATCTAAATGCACTAAGTGTGCATTGCC
TATGGTGGCCAGCAGAGGGTGCTGGGTCCCGAGGAGCAGGTTACCAGGAGCTGTGAGCCACCTGACTTGAGAGCAAGTGCTCCTGCCCCTTAGC
CCTCTGCTCCTGAGATCCTCATTTCTGAAGAGATCTGGCCACCACATCCTCGTAGGAAGGGATGGATGTTCACAGAGAAAGCCAGAAGACTTGC
AAGGTTCCCCACTCAAGACAATTACCATTAGGGTTCCCTTTGCCCAGTCACGTGTGCTGGATAGTTTTATATGTCAACTTGACACAAGCTATGT
CCATCACAGGAGGGAACCTTAATTAAGACAATTCCTCCATAGGACTGGGCTGCAGGCAAGCCTGTAAGGCATTTTCTTAATTAGTGATTGTTGG
AGAAGGGCTGGTGGTCCTGGGTTCTATAAGAAAGCGGGACTGGGCACATTCTTGGAAAGGGGAGGGAGGATGGGGAAGGAATGTGTGAGGAGGG
GACTAGGAGGAGATGGGGTGCTGCAAAGTGAATACATAATGGAAAAAATGAAAGGGCATGATGGTGCACGCCTTTAATCCCAGCACTTGGGAGG
CAGAGGCAGGCAGATCTCTATGAGTTCAAAGCCAGCCTGGTCTTCCAGAGCTGTCAGAGAAACTCAATCACAGCGGTGGGGGTTGGGGGAGCAG
GCTGAACAAGCCAGTAAGCAGCCTTCCATGGCCTTCACATCAGCTCCTGCCGCTAGGTTCCTTCCGTGTTTTGACTTCCTTCAATGATGGACAG
TGATGTGGAAGTATAAGCCAAATAAATTCTCTCATCCCCAAGATTCCTTGGTCATGGTGTTTACCACAGCAATAGAAACCCTGTGACATGTCAC
TGTGAGGTTGGTCCTCACACAGTACAGTCTCCATAAAAGCCCTAGCAAGCAAGAGGCCAATGTCTAACAGGAGAGTCCAAGATTCAAATCCTAT
AATCGGTGCAACCCCTTAACTCTTCCAAAACACCAAAACAGCTGCTGCCCTCTTCCAACTATCTCTAGAAATGTTCTTGCAGGTGGCAGCTGCA
GTAGGAGCTAATGACCCCCCAGATCTATGTAAAAACGAATCAGATGGTGAGGCTGGAGAGGTCGCTCAGTGGTTAAGAGCACTGGCAGCTCTCG
CAGAGGACATGAGTTTGGTTCATAGCACACACGTGGTAGCTCACAATCATCTGTAATTCCAGTTCCAGGGGAGGGATCTGTCACCTTGTTATGG
CCTCCATGGGCACTCCACACACATGGTGCACAGACATATATACAGACAAAATGCTCATACATAAGGTGAGATTTAAAGTCTCAAAAAAAAAGAT
TCAGTGTTTTCTGTGGGTAATGTTGCTCCGTGGATACAGCATGCATATGATGCACAGATGTGCATGCAAGCAAAACACATACGTAGAAATAAAT
CAGAAGTTAGAGATGGTTCAGTAGTTAAGAGCACTGGCTGATCTTCTAGAGGAGCCGGGTTCAATCCCCAGCACACACAGCGCAGCTTACAACT
GTTTGCAACTCCAGTTCCAGAGACTCCCACATCCTCACACAGACATACATGTAGGTAAAACACCAATGCACATGAAATAAAAATAATTAAAAAA
CATACTTATTGAGTACCTCGTAGTAGATTGAGGCATCTAAGAGGCTGCACGTCTCCCTGGAAGCAAGAGCTAACAGTGCCGATGGGCTTTTATT
CTTTTCATTTACACTCTTTCATTACACACTGGAAGGTGGGACAGACCTGCTCACTTCAGCACCCTGCCAGTTAAACAGAACCCCCTTCTGGCAG
AAGCCTGGTCCAAGTCTGGTGAACGTTTACAAGGAAAGCAGGCAGTCAGCAACTGAGCTTTATCCACAAGCACTGACTCTCAGATATAAACTGA
CAGTCGCTTTATCCTGGGAGCCTCCCTGGGAGCATCCAGTGACGTGTGTGTGTGTGTGTGTGTGTGCGTGTGCGTGTGCGTGTGTGTATAAGAG
AGAGAGGGGGGGGGGCCGGGTGTGGTGGCGCACGCCTTTAATCCCAGCACTTGGTAGGCAGAGGCAGGCGGATTTCTTGAGTTCGAGGCCAGCC
TGGTCTACAAAGTGAGTGCCAGGACAGCCAGGGCCACAGAGAAACCCTGTCTCGAAAAACAAAAACAAAAAGAGAGAGAGAGAGAGGGAGAGAG
AGAGAGAGAGAGTGTGTGTGTGTGTGTGTGATTGAGTGTGAGTGTGTGTGAGAGAGAAAAAGTGTGTGTGAGAGAGAAAATGTGTGTGTGTAAG
AGAGTGTGTGTGTGAGAGAGAAAAAGTGTGTGTATGAGAGAGAGAGAGTGTGAGAGTGTGTGTGTGATTGAGTGTGAGTGTGTATGAGAGAGAA
AGAGTGTGTGTGAGTGTGTGTACGTGCACACCAGCTCTTGTCTCTGCTCTTTGGAACAGTCCTCAGCTGTCTTGTGTTCACAATGACCCGGGAA
ACGTGCTCAGAACCCTGGGCCGCTGAGAAGAACCTAAGCCATGTTATTTGACAGCAACTGAGATGCAAGCAAGCTTTGCAGTAGTTTGTTAGCA
TGGCAGCTGAGTTTTCAATGCTCTGCCACATTAATTAGTTAATTAATTAACACATCAGCTCCTGCCACTAGGTTCCTTCCATGTTTTGACTTTC
TGTCTTGACTTCCTTCAATGATGAACAGTGATGTGGAAGTATAAGCCAAATAAACTCTCTCGTCCCCAAGATTGCTTGGTCATGGTGTTTACCA
CAGCAATAGAAACCCTGTGACATGTCACTGTGAGGTTGGTCCTCACACAGTACAGTCTCCATAAAAGCCCTAGCAAGCAAGAGGCCAATGTCTA
ACAGGAGAATCCAAGATTCAAATCCTATAATCGGTGCAACCCCTTAACTCTTCCAAAACACCAAAACAGCTGCTGCCCTCTTCCAACTATCTCT
AGAAATGTTCTTGCAGGTGGCAGCTGCAGTAGGAGCTAATGAGCCCCCAGATCTATGTAAAAACGAATCAGATGGTGAGGCTGGAGAGGTCGCT
CAGTGGTTAAGAGCACTGGCAGCTCTCGCAGAGGACATGAGTTTGGTTCATAGCACACACGTGGTAGCTCACAATCATCTGTAATTCCAGTTCC
AGGGGGAGGGATCTGTCACCTTGTTATGGCCTCCATGGGCACTCCACACACATGGTGCACAGACATATATACAGACAAAATGCTCATACA
MOUSE SEQUENCE - mRNA
CGCTCGCAGTGCTTAGCTCTTCTGTCGGAAACTGGTCTCTTTCCCCTTGCTGTTCTTCAACCCCTCTCTTTGGCCCTTGCTTCCTCACCTGCTC
TGGGACACCTAACTCAGAGACCTCCCTTCTCCCCCTGCCGGCCCAATTATGGCAGAGAACGATGTGGACAATGAGCTCTTGGACTACGAAGACG
ACGAGGTGGAGACAGCCGCTGGGGCAGATGGGACCGAGGCTCCCGCCAAGAAAGACGTCAAGGGCTCCTACGTCTCCATCCATAGCTCCGGCTT
CCGAGATTTTCTACTCAAGCCAGAGCTGCTCCGGGCCATCGTTGACTGTGGCTTTGAGCATCCATCAGAGGTCCAGCATGAGTCCATCCCGCAG
GCCATTCTGGGGATGGATGTCCTGTGCCAGGCCAAGTCAGGCATGGGAAAAACACCAGTGTTTGTCCTGGCCACACTGCAGCAGCTGGAGCCCG
TTACTGGGCAGGTGTCTGTGCTGGTGATGTGTCACACTAGGGAGCTGGCTTTTCAGATCAGCAAGGAATATGAGCGCTTCTCTAAGTACATGCC
GAATGTCAAGGTGGCAGTGTTTTTTGGCGGTCTGTCTATCAAGAAGGACGAAGAGGTGCTGAAGAAGAACTGTCCACACATCGTCGTGGGGACT
CCTGGCCGAATTCTAGCCCTGGCTCGAAATAAGAGCCTGAACCTCAAACACATTAAACACTTTATTTTGGACGAGTGTGACAAGATGCTTGAAC
AGCTCGACATGCGTCGGGATGTCCAGGAAATTTTTCGCATGACCCCCCATGAGAAGCAGGTCATGATGTTCAGTGCTACCTTGAGCAAAGAGAT
CCGCCCAGTCTGCCGCAAGTTCATGCAAGATCCTATGGAGATCTTCGTGGATGACGAGACCAAGTTGACGCTGCACGGGTTGCAGCAGTACTAC
GTGAAACTGAAGGACAACGAGAAGAACCGGAAGCTCTTTGATCTTCTCGATGTCCTCGAGTTCAACCAGGTGGTGATCTTTGTGAAGTCCGTGC
AGCGCTGCATCGCCCTGGCCCAGCTTCTAGTGGAACAGAACTTCCCAGCCATTGCTATCCATCGTGGAATGCCCCAGGAGGAGAGGCTCTCTCG
GTATCAGCAGTTCAAGGATTTTCAGCGGAGGATTCTTGTGGCTACCAACCTGTTTGGCCGAGGCATGGATATTGAGCGTGTGAACATTGCTTTC
AACTATGACATGCCAGAGGACTCGGACACCTACCTGCACAGGGTGGCCAGAGCGGGCCGGTTTGGCACCAAGGGCTTGGCCATCACATTTGTGT
CAGATGAGAATGATGCCAAGATCCTGAATGACGTTCAGGACCGTTTCGAGGTCAACATCAGCGAGCTGCCCGATGAGATTGACATTTCCTCCTA
CATTGAGCAGACACGGTAGAGGACTCGCGTGGTCAGTCTGCTGTAGAAGAGGACACGGGTCAGGAGGAGACACTACCGCCCCACCCGACACCGA
CGCCTCTGCCCACCCTATCTATGCTTCTCTCTGCGTCACCACTCCTAAACCTAGTCCTGATTTATCAGAGTTGTTTGTTTGTTTGTTTTTGTTT
TTTAACAAAACTAAGAATGAAAAAAAAAAAAAAAAA
MOUSE SEQUENCE - CODING
ATGGCAGAGAACGATGTGGACAATGAGCTCTTGGACTACGAAGACGACGAGGTGGAGACAGCCGCTGGGGCAGATGGGACCGAGGCTCCCGCCA
AGAAAGACGTCAAGGGCTCCTACGTCTCCATCCATAGCTCCGGCTTCCGAGATTTTCTACTCAAGCCAGAGCTGCTCCGGGCCATCGTTGACTG
TGGCTTTGAGCATCCATCAGAGGTCCAGCATGAGTGCATCCCGCAGCCCATTCTGGGGATGGATGTCCTGTGCCAGGCCAAGTCAGGCATGGGA
AAAACAGCAGTGTTTGTCCTGGCCACACTGCAGCAGCTGGAGCCCGTTACTGGGCAGGTGTCTGTGCTGGTGATGTGTCACACTAGGGAGCTGG
CTTTTCAGATCAGCAAGGAATATGAGCGCTTCTCTAAGTACATGCCGAATGTCAAGGTGGCAGTGTTTTTTGGCGGTCTGTCTATCAAGAAGGA
CGAAGAGGTGCTGAAGAAGAACTGTCCACACATCGTCGTGGGGACTCCTGGCCGAATTCTACCCCTGGCTCGAAATAAGAGCCTGAACCTCAAA
CACATTAAACACTTTATTTTGGACGAGTGTGACAAGATGCTTGAACAGCTCGACATGCGTCGGGATGTCCAGGAAATTTTTCGCATGACCCCCC
ATGAGAAGCAGGTCATGATGTTCAGTGCTACCTTGAGCAAAGAGATCCGCCCAGTCTGCCGCAAGTTCATGCAAGATCCTATGGAGATCTTCGT
GGATGACGAGACCAAGTTGACGCTGCACGGGTTGCAGCAGTACTACGTGAAACTGAAGGACAACGAGAAGAACCGGAAGCTCTTTGATCTTCTC
GATGTCCTCGAGTTCAACCAGGTGGTGATCTTTGTGAAGTCCGTGCAGCGCTGCATCGCCCTGGCCCAGCTTCTAGTGGAACAGAACTTCCCAG
CCATTGCTATCCATCGTGGAATGCCCCAGGAGGAGAGGCTCTCTCGGTATCAGCAGTTCAAGGATTTTCAGCGGAGGATTCTTGTGGCTACCAA
CCTGTTTGGCCGAGGCATGGATATTGAGCGTGTGAACATTGCTTTCAACTATGACATGCCAGAGGACTCGGACACCTACCTGCACAGGGTGGCC
AGAGCGGGCCGGTTTGGCACCAAGGGCTTGGCCATCACATTTGTGTCAGATGAGAATGATGCCAAGATCCTGAATGACGTTCAGGACCGTTTCG
AGGTCAACATCAGCGAGCTGCCCGATGAGATTGACATTTCCTCCTACATTGACCAGACACGGTAG
HUMAN SEQUENCE - GENOMIC
ATCTTTTATTATCACTTATCAATAAACTGAGTAAAATAAATGTTTTCAGGGGAATTTCTCTCAGCCAGCCTTACCAGGGGATGATGGGAGAGGG
GTGGGGAGGTGAACCGGCAACAACTATGGCCGGCGGCAGAGCAAGCTCTTTCCAAATGACTGCTGACCTAGGGCAGGGGAAAGGGAGTGGAGTG
TGACAGAGGGTCTCACCCATGGGCTGAGAGAAAACAGGAGAGGAACCGACGTTCCTGAACTCCCCTTTTCTTCAGTCCCAACCTTGCTGCATCT
GGCCCAAGGTTAGCTGAGTGCCATGCTACTTCCTTCACTGCCAACCCAGGCATCCTGGCCAGGCCCACCTGCTGTGGCCACCAACCACCTCTTT
CACTTGGGGGATAGAAGAAGGGGAGGGAGGCAGCCTTCCTTCCTGTGGACCTACTTTCTTTCCCCGGGGTAAGAGGAAATGGGCTAGCAGTCCT
TAAATCTTTATTTGGTAGTGCTGGAAAGTACTGTTTACCTGGCAGAAAGCTGGAATAGGGGAAGGCAAGGCCAGGAAGGCAAGAAGACAGAATG
GCCCAGGTGTGGCTGGCGAAGGCCCACCATCCCTACCCAAATCACATCAGGGTTGGTGGGGGGGGCACTTCTCCCTAGTGCTGCTGTGACCTGT
CACAGACCCTCTCAACTTGTCCCACCCAGAAAGTACCTGGTCCTGTCTCTCATTCGCTTGTTCCCCACCTGAGCTCAGGTGGTGAGCATGGTGA
GTGCTCAGGCTTGCATGGGAGGTTTACATTCATAGGTTTTAAGGAGTAGGGCCTCCAACTATAAAAACATAATATTAAACAGCCACTACAACTG
AGGCATGTGTTTGAAAAAAGCTGGCTACAAAACTGTAGAGAGGATCAGATGTGGCCAGGCACGGTGGCTCACGCCTGTAATCCCAGCACTTTGG
GAGGCTGGGGGGGGGGACAATGGATCACAAGGTCAGGAGTTCAATACCAGCCTGGCCAAGATGGTGAAAACCCGTCTTTACTAAAACAAACAAA
CAAAATATATATATAATTATATTTTATATTATATATAAAATTAGCCAGGCGTGGTGGCTGACGCCTGTAATCCCAGCTACTTGGGAGGTTGAGG
CAGAGAACTGCTTGAACCTGGGAGGCGGAGGTTGCAGTGAGCCGAGATCGCGCCTCTGCATTCCAGCCTGGGTCACAGGGTGAGACTCTATCTC
AAAAAAAAAAAAAAAAAAAAAAAAGAGAGAGAGAGAGAGGATCAGATAATAACTGTCTAAAACAAGAGACCAAATCCTATGGTTGGAAAAAAAG
AGGCCGGGCGTGGTGGCTCATGGCCTGTAATCCCAGCACTTTGGGAGGCTGAGGCGGGTGGATCACCTGAGGTCAGGAGTTCGAGACCAGCCTG
GCCAACATGGTGAAACCCTGTCTCTACTAAAAATACAAAAATTAGCCAGGCGTGGTAGCATGTCCCTGTAATCCCAGCTACTTGGGAGGCTGAG
TCAGGAGAATCACTTGAACCTGGAAGGCAGAGGTTGCAGTGAGCTGAGATCATGCCAGTGCACTCCAGCCTGGGCAACAGAGGGAGACTCCCAT
CTCAAAAAAAAAAAAAAAAAAAGAAAAAAAATAGACTGGCAAAAAATATGTAAAAATTCTAGCCTATGGGTGGTGGGGCTGTAGGTGGCTTTTC
CCCCAATTTTATTTGACATTTTAATGTGAAAGGGATATAATTAAGTTGAAATTTTCTTTTTAAAGAAGGACGAACAAATTTGATAACTATTAAC
AGTGTACTGTGATCCCTTCCAGTCCCACCTTGGTGTACCTAAAAGCATACATATGACACATTTATTTGGAGATATGTAACCATACTACAAATAT
TGCTTGGCAACTTGCTTCTTTCATTTAACATCTCATTTATATTTTTCCACAGGAATATATACAGTCTACCTCATACTTTTTGACGACTTTATAA
TTAGTGTTCCACTGTGTGTACAAAACAAATCTCCTTACCCCAATATCGATACACTTTGTTTCAAAAAATGTTCACCCTCATGAGTATCTTACAG
TCTTTCTGTGGGGGTAGATTTTCATAAGTGGAATTTCTGAGTCAAAGGACATGCGCAATTTGGTCAAACTGCTCTCAATAAGTTTGTGCCAATA
TACAAGTGTGCCTATGTCTTCCTTTACCAAAATTGAATATCATCAACCCTTTTAATTTTGCCAGTTGGATAGATTAAAAAATTATTTTATTAAC
AATGTTTTTTTCTTTTTCATGTCTTTTGGCTATTTGTATTTTTTGTGAATGAATTGCTCATATTCTTTATCCACTTTTTCTTTGGAAATTATAT
ATATTGATATGAGGGTTCTATATACTGTATGTGTTATACATATTGCAAACATTTTCTTCTATCTATCTTTAAAAAAGCTTTTTTTTTCTTTTTG
AGACAAGGTCTCACCCTGTTGCCCAGGCTGCAGTGCGGTGCCATGATCTTGCTCACTGCAAACTCTGCCCCACCTGGGCTCAAGCGATCCTCCC
ACCTCAGCCTCCTGAGTAACTGGGACTACAGGACGACAGCCACGTACCACCACGCCTGGCTAATTTTGTATTTTTTGTACAGACCAGGTCTCAC
TGTGTTGTTCAGGCTGGTCTTGAACTCCTGGCTCAAGTGATCTGCCTACCTTGGCATCCCAAAGTGTGGGATTACAGGTGTGAGCTACCGCACC
CGGCCTAAAATTTTCATGTAGTTAAATCTATATCAATATATTATTTTCTGACTTTTTATTTCACTTCACTCTTAGGAAGGCTCTATTTTAAGAT
TATAAAACATTCTCTGCATTTTCTTCTACTACTTTAGATGTATACTCATTTTTATTTTTAGAAATTAAAAAACTCGGCCAGGCGCGGTGGCTCA
TGCCTGTAATCCCAGCACTCTGGGAGCCCAAGGTGGGTGGATCATGAGGTCAGCAGTTCAAGACCAGTCTGGCCAACATGGTGAAACCCCGTCT
CCACTAAAAATACAAAAATTAGCCTGACGTGGTGGCGGATGCCTGTAATCCCAGCTACTTGGGAGGCTGAGGCAGAGAACTGCTTGAACCCGGG
AGGCAGAGGTTGCAGTGAGCCAAGATTGCGCGACTGGACTCCAGCCTGGGTGACAGAGTGAGACTCTGTCTCAACAACAACAAAAAAGAAATTA
AAAAACTCTCAAAAATACAAAGTATAATACAATACACAACCCCATTCCTACCACAGGGTTTGTTAATGTACTGTCATGTTTGTAGTATAATTTA
TTTTAAGGAAATAAAATCATCACAGATAAAGGTAGTTTACCGTGATACCGCCTTCCAAGTTTCATTCCTCCCCAGGCCATCTGTCCCATGAATT
TGGTGTGTACCTTCCTGTCATATTCAGGTATTTAATCTGCTGGAATTTACTTTTTGATTAGGTGTGAGGTGGGACTCTTGTTTTTCCCTAGATG
AGCCAAATGTTCCAATATTATTTATAAAATAGTTCACCTGGTCAGGCACAGTGGCTCATGCCTGTAATCCCAGCACTTTGGGAAGCTGAGGTGG
GTGGATCACCTCAGGTCAGGACAGGAGTTTAAGACCAGCCTGGCCAACACGGTGAAACCCCCATCTCTACAAAAATACAAAAATTAGCTGGGCA
TGATGGCAGGTGCCCAGCTACTCAGGAGGCTGAGGCAGGAGAATTGCTTGAACCCGGGAGGCGGAGGTTGTAGCAAGCTGAGATTGCGCCATTG
CACTCCAGCCTGGGTGACACAGCGAGACTCTGTCTCGGAAAAAAAAAAAAAAAAAGTTCACCTTTCTCCAGTATTAGAAATGCCCCCTTTAGGC
CTGGCGCGGTGGCTCACGCCTCTAATCCCAGCATTTTGGGAGGCCAACGCGGGCGGATCATGAGGTCAGGAGTTCGAGACCAGCCTGGCCAACA
CAGTGAAACCCTGTCTCTACTAAAAATACAAAAAATTAGCTGGGCGTGGTGGCGGGCGCCTGTAATCCCAGCTACTTGGGAGGCTGAGGCAGGA
GAATGGCTTGAACCTGGGAAGCGGAGCTTGCAGTGAGCCGAGATCACACTACTGCACTCCAGCTTGGGCGACAGAATGAGACTCCATCCCCCAC
TTCGCCAAAAAAAAAAAAAAAAAAAAAAAAAAGAAATGCCGCCTTTATAGCGATTTACCAGATCAACCGTTCTCAATGCTCTTTAATACGCTGG
AGTTTCATACTAAGAAAAATAAACATAAAAACATTTTGGCCAGGCGCTGTGGCTCACGCCTGTAATCCCAACACTTTGGGAGGCTGAGGTGAGC
AGATCACAAGGTCAAGAGATCGAGACCATCCTGGCTAACATGGTGAAACCCCGTGTCTACTAAAAATACAAAAAATTAGCCAGGCGTGGTGGCA
TACGCCTACAGTCCCTACGCCTATAGTCCCAGCTACTTGGGAGGCTGAGGCAGCAGAATCTCTTGAACCTGGGAGGTGGAGGTTGCAGTGAGCC
GAGATTGTGCCACTGCACTCCAGCCTGGGCGACAGAATGAGACTCCTTTTCAAACAAAACAAAACAAAACAGAAAACAAAAACAAAACCAAAAG
ACATTCTGTGGGATGGGCACGGTGGCTCATGCCTATAATCCCAACATTTTGGGAGGCTGAGGTGGGTGGATCACTTGAGGTCAGGAGTTTGAGA
CCAGCCTGGCCAACATGTTGAAACCCCATCTCTACTAAAAACACAAAAATTAGGTCGGGCATGGTGGCTCATGCCTGTAATCCCAGCACTTTGG
GAGGCCGAGGCAGGTGGATCATCTAAGCTCAGGAGTTCGAGAGCAGTCTGGCCAACATGGTGAAACCCCATCTCTATTAAAAATACAAAAGTTA
GTCGGGCATGGTGGCAGGCTCCTGTAGTCCCGGCTACTCAGGAGGCTGAGGAAGGAGAATCACTTGAACCCAGGAGGCGGAGGTTGCAGTGAGT
CAAGATACTGCCACTGCACTCCAGCCTGGGGAACAGAGGGAGACTCCGTCTCAAAAATAAATAAACAAATAAAAATTTAAAAATTAATAAATAA
AAATAAAAAAATTAGCTGGGCATGGTGGTGTGTGCCTGTAATCTCAGCTACTTGGGAGGCTGAAGCAGGAGAATCGCTTGAACCCAGGAAGCAG
AAGTTATAGTGAGCCGAGATCATGCCACTGCACTCCAGCCTGGGCGACAAAGCAAGACTCCGTCTCAAAAGGAAAAGAAAAAGGAAGCTGGAAG
CTGAATGAGATGGGCCTTTCAACCAAGGAGTTAGAAGGCCATCTGGTGGCAGGGCTGGCAGAGGACCAGGAGTAAATAAGGCCAGAGAGGACAC
CAGGGTCTGGGAGTGAAGGCACTGAGCTTGGGTCCCCCTTTGGAAGACAATGACCTGAGAGCTGTGAGATTTCAGACAAGTTCCCGAACCTTTT
GGGCCCTGCTTTCCTCATCTGTAAATGGGATAATATCAGTCTCACCAGCTTCTTAAAATTCAATACAATGGAGTTGGGTGTGGTGGCTCACGCC
TCTAGTCCCGGCACTTTGGGAAGCCGAGGTGGGCAGACTGTTTGAACTCAGGAGATGCAGAACAGCCTGGATAACATAGCAAAACAGTCTCTAC
CAAAAATACAAACAATTAGCTGGGCATGGTGGTGTGTGCTTGTAGTCCCAGCTACTAGGGAGGCTGAGGTGGGAGGACTGCTTGAGCCCACGAG
GTAGAGGCTGCAGTGAGCCATGATTGCACCACTGCACTCCAGGCTGGGAGACAGAATGAGACCCTGTCTCAAAACAAACAAGCAAACAAACAAT
AAAGGAAATCCCTACCACACTATCAGGGGCATTTTGGCTGAGCGCGGTGGCTCACGCCTGTAATCCCAGCACTTTGGGAGGCTAGGCTGGCAGG
TCACCTGACGTCGGGAGTTTGAGACCAGCCTCACCAACACGGAGAAACCGTCTCTACCAAAAATACAAAATTAGCCGGGCGTGATCGTGCATGC
CTGTAATCCCAGCTACTTGGGAGGCTGAGGCAGGAGAATCTCTTGAACCCAGGAGGCAGAGGTTGAGGTGGGCTGAAATCGCGCCATTGCACTC
TAGCCTGGGCAACAACAGGGAAACTCCATCTCAAAAAAACAAAACAAAACAAAAAACAAAACTCCCATTTTTGCGAGGCAAATTGGGCTCACAG
AGGTAAGCTGCATCTCCCTGTTGATGGCAGAGCTGGGGTCTGGATGCAGGTCTGCTTCGGGGTAATCTGCTCTTTTGCCTTCCAGGGTCCTGCC
TCTTACAATATGAGCTGTCAAGTTAGATGCCTGCACTCAGTAAACCTACTCTGTTTTAAGTAAAAACAACAAGAAACAAATCTGAATATGCTAG
CCTATCTCAGGTACGTTAAAGGAAATTTTTAAATAGGGGGTTTTTTGACCATTTGGGGGAGTTTTGGGGGAGGGGCCTTCTGTCTATACTTGAG
CTGGGGGATGTTAGGGTTGTTCATCTGGATCTAGAGGTTTTCCTGTAATGTTCTTACTCCAGAAGGAAATCTCTAGATGGGGAAAGAAGGTTTC
AGCTTTTATTCTAGTAAGCAGGGCTCTACCTATAAAGAGCTGCTTCCACCACTCTTTTTTTTTTTTTTGAGACGGAGTCTTGCTGTGTTGCCCA
GGTTGGAATGCAGTAGTGCAATCTCGGCTCACCACAACCTCTGTCTTCCGGGTTCAAGTGATTCTCCTGCCTCAGCCTCCCAAGTAGCTGAGAC
TACAGGTGTGTGCCACCATGCCTGGCTAATTTTTGTATTTTTAGTACAGATGGGGGTTTCACTATGTTGGTCAGGCTGGTCTCGAACTCCTGAC
CTCGTGATCTGACTGCCTTGGCCTCCCAAAGTGCTGGGATTACAGGCATGACCCACCGCACCTGGCCTCCACCACTATTATAATATCACCAGGT
TCCCCATTTGAATCCTTCAGTGCCAAAGGTTTTGCAGAATTCAAATGTTTTTGGGACTTGATAGGGCTGACCTAAAAGTACACTCACTCTATAT
TAGGTAGCCCAGGAGGGCCTAGGCAGCCCAAGAACCAAACACATGAGTGTTTCTGCAGGGAAATGTATGAATATTCACATCAGTAGGATGAAAA
TAAATAATAGTCTTACTTTAGTTCAGATTAGGTTTCTGTCACCAAATGAATTTTGGTGGCAGCCTGATGAAAAATGTTGGTTCTCAGAGTGTTT
TTGAGTTTAGAATTGTGGTTAACGGAGTATGGACCTGTTGATAACAAAAACAGGAACAAGGCCAGGTGTGGTGGCTCACACCTGTAATCCCAGC
ACTTTGAGAGGCTGAGGTTGGTGGATCACCTGAGGTCAGGCATTCGAGATCAGCTTGTCCAACATGGCGAAAACCCATCTCTTCTAAAAATATA
AAAATTAGCTGGGCGTGGTGGCATCCGCCTGTAATCCTAGCTACTTGGGAGGCTGACGCATGAGAATCACTTGAACCTGGGAGGTAGAGGTTGC
AGTGAGCCAGGATCGCACCATTGCATTCCAGCCTGGGCAAGAAGAGTGAAACTTCATAAAAAACAAAAACAAAAACAAAAAACAGAGAAACAGG
AACAACAATCGCCAGCATATACCAAGTGCTTATCGTGTGTGCCAGGTACTCTAATTATGTACTATGTCAGTTGATTCTCAAAACATATATGGCA
CAACATGGGTACTCTGAACATGGCCACAATCAATGTACAATGCTATAATGTATAACACAGGACAATGTAGCTGTTAAAAGCATGGACACTCTAT
CTAGTCCATCTGGGTTATAATCTCTGCTCTACCAGTGAATAACTGTAACTCTGGCAAATGACTTCTCTATGCCCTGTTTCCTCAGCTGGGAAAT
GGGGGATAATATCAGTACTCACCTCCTAAGATTGTTGTGAGGATTAAATGTGTTACTTTATAAGAAATGTCTGGCACATAGCAAAGGTTGTTAT
TATTTTAATTTTTACACATGGGCAAACTAAGCCTCAAGTAACCTGTCCAAGAATACGTAGCTATGAAGTGTGGAGCTGGGATTTGGAGCTGGGG
TTTGAATCCAGGCAATCTAACTCCAGAGCCTACCTTCTATGCTACTTTTTGGCTACGAGCAAACAATCTGTCAAGAAACAAAGTAGCTACTAAT
CTAAACAGATGTGAAATTTGAAGACCAGTTGATCTTTGGGGAATGTTGGGTTCTTCAGACAATGGTAGCTCAGTAATGTAAAGGGACAAATGAC
AGCCACATGCCAACTTGGTTAACTCCTTCCCTAGGTCCTGATGACCAAATAACCAGCTTATTTCTCAACTATTGGTTGGCTTTCATTTCAGGTC
TGGTCAGCTGCTTATGACCTTGTTCCCCACTGAGCAGACTCACCATCTGGGCCCTGGCGGGCAGCAGCATGCAGTGCCGTGTCCCCATGGCGGT
CCTGGTGGGCAGGGTCAGCCCCGAGCCGAAGCAGCAGGCACAGGGCAGGGGCATCGTGGCGGGCACAGGCCCGCTGCAGTGGTGGGGGCTGCCC
AGCATCTACATCCAGGCCTGGGTGTCGCTGGAGGAGGGCCTGGGCCCGGACCAGCCGTCCTGCAGACAAGTAACGACGAAAGCGACGTTCTCGG
CGTTGGCGGCGGGAAGTGGAGGCCATGGAACTCTTGGGCTGGGGAAGGAAAAAAGGCAGCAGTCAGGACTTCAGCCTTGGCTGGTCCTTCTCCC
TCCATCTCTGACATCCCCTGTTGTTTCTCCCTTTGGTTCCGTCTTTTTTTAATATCTTCAGCAAGAGATGAGGCCTAACCTAACCCTGATCCTT
TATCAGATGATAGATTTGAAAAAAAATTTTTTTTGAGGGGGGTGATAGGATCAGAGGTTTAATTTTTTTAATGTAAAATTCGAGAAAAGGGTAA
ATAATTGGTTTAAGGCTCAGGAGCCCAGGTAAATTTTTAATTTTTAACAAAGAACTTTAAAAAAACCCAACAGGGCCGGATGGGGAAAATTTTT
ATCAGCAGAAATCTGAGTTTTAAAAAGTCACAGATAATCTCCAATAATGATCTAGAAATTGAATATCATGTACCCGGCAGACAGATGTGGAGGC
TTCTTCCTCTGGAACCTGGGGGGAGGGGTTACTCATCAGACCTGCCCCCGCCCCCCCAAGTACCCCCAGAGCCGTAGGCCCAAGGCCTGTGTTT
AAGAAGCTCGGAGACGGGAGGCGGGAAGGGCGGAGACACTCCAGGCTGGAGGAAATGGCGCAAGCAGAGACGCAGGTGGAGGACGGAAGTGAAC
TGTGAGGGGCGTTACCGGATGTCGTTCCGCCCCGACCGGGTAGTTCTTGGCCAGATCTCCCAGGGGAAACTAGGGAACTTAAATTAAAGGGGCC
GTCTGAAACCAGAAGACTGGACTGGAGGCGAGGAAAAGGAGGCGAGGGGAGGGGAGGGAGAAAAGAGAGTTATTTGGAGGTTTTTCCCCGCCTC
CTCTAACTTGGCAGAGAGAGGAGATGGTTCAGTGATGGACGAAAAGATGAGAAGACAGAGAAAATAGAGGAGATAAAGACAGGATAAAAATCAC
ATTAAACATGGAAAACAAAAACAAAAACCACAGTGGGACAACAACAGGGACAGATCAAAAAAAAGAAAAAATACAGACAAAAGACGGAAGAAGA
CTATCGTAGGATGGGGCAAGTGAGATGCAAAAATTTGGACTTGAGAAATATGTAGAAAAAGATGGAGATGTTAACAACGGGAGGCAGGGGAGGG
GGCGGGATGGTGGAGAGAGAGAGAAAGGTAGAGAGTTAGTTTAGAATTAAGCCCAGAATGCTCTTTTCCCAACACAGGTTGCATGATGACATCC
TTACCTTTTCACCGCATTCACAACCCTTTATGCTTCCTCTGTTACCACCAATCAAGTTCTCCTTCTCTCACCTCAGTACTCCCCCGTCTCCGCC
CCTGCCTCATCCCTAGACCTTTCCGACTGGGATGGCTAACCTGTTGTAAGCCCGCAGCTTTGGGCCTGGTCTCTGCTGCTCCCAGGCCGCCCCT
TTGGGTACTGCCTGAGCAAGAAGTGCTGGAGAGGAGGACCAGTCATCAATAGGAGGATGAGATTGGGAGAGACACTCGGTGCAGGAGGCTGAGT
GAGCAGGGGAGCACTAAGACCCAGGGGTAGTGGAGCACTGCAGCAACGAGCTGGAGGAGGAGAAGTAAGCGGTGGGGGGTGGGAGCCATCTGGT
ACTTTGACAGCATTCAAAACAGCATCGGCCATAACAACAGAAATGGCCAGTCAGTCCCAAGGTATCCAGCAGCTTCTGCAAGCTGAGAAGCGGG
CAGCTGAGAAGGTGGCAGATGCCAGAAAGAGTGAGTCTCCTCTTTCCTCCCTTAGGAGTTTGGAAAGAAAATTGGGGGTGGGGGACAGCAAACA
TTTTGGGAAAACCCAAGCCTGGCGGGAACACAGCTAGGGTCTGGAGGCTGGTTAGGAGGGAAGAAATGGATGGATATTAGAATCTGGCACCTGG
TTGGCTGAGAGAAGGCTGTATAACTTTCTGGAAGGGACTGACTCCTGCTATTACATTGTGTGTGTGTGGGTCCATCCCCACTCACTGTCCTTTC
TTCTGCCTCCAGGGAAGGCCCGGCGACTGAAGCAGGCAAAGGAGGAGGCACAGATGGAGGTGGAGCAATACCGCAGAGAGCGAGAGCACGAATT
CCAGAGCAAGCAGCAGGCGGTGAGTTGAGGCAGAGTCGGGATGAGACCCCACTGCAAGTTGGTGGGTGCATCTAGTGAGGTGTGTAAGGGTGAC
TCAACAAGAAAATATGGTGGCAGAGGGCTGAGGCTGAGGGGACCCTGGCAGGGACCACAACATTGGTGAAACTTTGTGATGATATGTAGGAGAG
TCTCGGAGTTTTGAAGGCCACATAGAGCTTGTGGGCGGAATGCCACAGTCTGTGTAAAGTATAACATCTATGTGGAGTATGATTAACATTTGTG
GTGGAGGGTAGAGTTTTATGGTCATGGATGGTGAGGTGGTGGGGATATTACGGTCTGTTTTAGGATGAAGTTGCATGTTAGGTCTAAGGGGAAA
GGGGACTGTGTTGATCTCTTTGGTGTTGGGATATTTCTGTGGGATGGGGGTGGTTTCTGAGAGGGCCTTTCTTCTAGGCTTTGTTTCAGGATCT
TTCCCCTCATATGCCTGGACCCTTGTCTGTTTCTGCTTTTCCCTTTCTCTCTTCCACCCCTCTCCCTACCCCCCAGGCCATGGGCTCCCAGGGG
AACCTGTCTGCTGAGGTGGAGCAGGCTACAAGGCGCCAGGTGCAGGGCATGCAGAGCTCCCAGCAGAGAAACCGAGAGCGTGTCCTGGCCCAGC
TTCTTGGCATGGTCTGCGACGTCAGGCCCCAGGTCCACCCCAACTACCGGATTTCTGCCTAGGGCCACCGTAGGGCCTGACTCCTTCTGCCAGT
TCCCTCCCTCAAAGAAATCCTCCAATCAAAATCACCTCCCACCATAATCCCTGTCTTCTTTCCATCCCCTAGAAATCCTGGGAGGCAGGATCCA
ATAATTTTCCTGTGACACTTATAAATATCCTGCTCACATCTGAATCTCCTTGTTGTTCTTTAACCCTCACTGGGACTTTGTAAACTTCCAAGTC
ATTCTCACCTAAACCCTCTGTGAAATTTGTAATATGGGGAAGTAGGAATGTGGAAAACATCCTGACTTCAGTGTCTGGCCGATGTGGGTCCCTC
TCTTGACCCTGTCACTTGCTGGCTGTGAAACCAGGACAAGCTACTTAACTTGGTAGCCTCGATGTCCTCCTCTGTGAAACTGGGATGATAATAA
TGCCTACCTTGTGAGGGTTGCTTCAATGATTAGGAATCATTCTGTAAAGTCTAGCACAGTTCCTTGCATGTTGTAGCAGTGATTCAGTAAGTAG
CAACCCTGTGATACTATTACCACCACCTGCTCACTGGTCAAAACCTACACAGCTGTTTCCTCACGTCCATCACTGGCTCTCTAATTCCACTTGT
TCATTCTGTGACCCTAGTTATTTTCTGAAAAATTGGTTCTTCTCTTTTCCCAGAGACCTTCTGATCTCCAAAAAGAGGAGATGACTACATTTAG
CCCCTCTCTTATAATTCCAGGTAGATAACTGCATTTTGTAGCCTCTCTTTGTTTTTCTTTTGCTGATCTTTGTCTTTATTAGATTTTCCTCCTT
TCCTATTTCCCCAAAGACTTATCAGATGCTCATTGCTTTCTAAGATCTAAAATGATACTGTGTTCCCTCATATGCATGCCCTTCCTTTCTATAT
CCTTCACACCTTACTTTCCCATTGTAACAATAAAAAAAGTATCAATAAAATAATTATTGGCAAATAAATTGGTGAGTTGAAGCAGCCTCCTTTT
GCCTCATCATTTCTCATTTTCAGTCACTTTGTTTTTTTTTTTTTTGAGATGGAGTTTTGCTCTTGTTGCCCAGGCTGGAATACAATGGCGTGAT
CTCAGCTCATTGCAACCTCTGCCTCCCAGGTTCAAGCGATTCTCCTGCCTCAGCCTCCCAAGTTGCTGGAATTATGGGTGTGTGCCACCACGCC
TGGCTATTTTTTGTATTTTTAGTAGAGATGGGGTTTCGCCATGTTGGTCAGGCTGGTCTCAAACTCCTGACCTCAAGTGATCCACCTGCCTTGG
CCTCCCAAAGTGCTGAGATTAGAGGTGTGAGCCACTGTGCCTGGCCTTCAGTCACTTTCTTGTTTTTTGTTTACATATTCCCTAAACAGCCCAA
ATGGCTATCCTTTGAAACTTCTTGGAGAAACAAGAACAAGTAGTACTTTATTATTTCTCTAAAGTGAGAAACATGGTTCCTCATTTGGGAATCT
GAGGACTATAGATCGCAACTGTAGAGAAAAGCTGGAGTGTAGGAGCAAGTGCTCTTTGCCCCTTTACCTTGCATTTTCTTCATAGCACTTACTG
CTACTGGTTTTTTGAGACAAGGTCCTGCTGTGTTGCCCAGGCTGGAGTTCCAGCTCACGGCAGCCTTGACCCCCTGGACTCAAATGATCCTCCC
ACTTCAGCCTCCTGAGTAGCTGGGATTACGGGCGAGTGCCACTATGCCTTGCTAATTTTAAAATTTTTTGTAGAGATGGGGTCTCACTTGCCCA
GGCTGGTCTGAAACTCCTGGGCTCAAGCAATCCTTCGGGCTCGGCTTCCTCAAGGGTTGGGTTACAGGCCTGAGCCACTGCACCCTGACCACTT
ATCGATACTTGACATTATATTTGTGTTTATGTGTTTTCTTTCCTGTAATGTAAACACTGTGAGAACAGGGCTGTTCACCGTTGTGTCCCCAGAT
CCTAGGACAACATGTGGCACAAGGGAGGCAGTTGATAAATACTTTTGAATAAATTAAATGATACTTGGGAAAATACCTTCTATCACACCATTCT
TGAATTAGTTACTTCATTTGTCACTGAAGACAAGCTTACTTCACCAAGAATTTGAACCAATAAGGTAACCTGCAGTGTATTTACTAACCAGATT
CTTTGAGCAGGGAGGCAGAATACAATAGAGAATGAGAGATGTTTGCATCCTGGCTGTAACCTCACCAGCCGTACTGCTTGAGATATGTTGCTTT
GCTTCGCTTCTGTCAATAAGATGAGAATAACGGTACCTACTCCTTAGTATTAAATGATTAAGTATGTTAACAGGGAGAGGGCCAAACGTTTGTT
GTTTTATTACACAGCAGGACATCAGGTCTTACTTTTGTGGCTCCCCATCTCAAAGACGGGGATAGCAAATGTTTCATTCAGGAAAAAAATCCAG
GTTGAACAATGGGGCTGTTGGGGCGGGGCCAAGAACATTCTGCTCGAATTAACAGTATTAATGGGCCGGGCGCGGTGGCTCACGCCTGTAATCC
CAGCACTCTGGGAGGCCGAAGTGGGTGGATCACCTGAGGTCATACATGGGTGAAGCCCCGTCTCTACTAAAAAAACAAAAATTTGCTGGGCGTG
GTGGCGGGCGCCTGTAATCCTAGCTACTCGGGAGGCTGAGGCAGGAGAATCGCTTGAACCCGGGAAGCAGCGGTTGCAGTGAGCCGAGATCAGG
ACATTGCACTCCCGCCTGGGCCACAGGGCGAGACTCTGTCTCAAAACAAAAACAAAAACAGTATTAATGGAATGTAGTATAACCCTCAAGCCCT
ACTATTAACACTTGGGGCCGAATCCAGACCCCGTCTTCCCGCTCGGATTCAGAACACCTTCCTGACTCACTGGCCCTAGGGCATCAGCTACCTC
GGACAGCATCCTTTTGGGAAAATACCGCCCACCAGCCCCACGACTGGGAAAGAGTCGGGAAACACCCCCGAGCAATCCAGTTCCCTGAGACTTC
CCTCCTCCCTCCCCTCAGCTAGGGCCTGCCGGTTCCTAGTGCGTGCCCAGCAGTCCTCAGGTCACCTTCACTACCGGGCCAAGGACCCCGTGGG
AACTCGCAGCCTTCGCCACACTCGTTCCTCGCGCATCCACGGAGGGGTGCCTACAGAGAAGACCTGCGTGGCAAAAACCTAAACGAAGAGATGA
GGGGCATGGAGAGGAGTAGGATAAGAGAATAAAGATAACAGTGGGGGGGAGACGTTAGTTTCCTTTATATCTTTTGTTACTGGAGGTAGCAGTG
AAGTTAGAAACGGTTTTAAAACAAATTTCAGACAGGCATTTTCCAAAGGCAAGCCTGGAGCGCACGGATCTGTATAACCGCGGAAGCCCCTGTT
TCCGGTCCCTTGCGCCTGCGCTCTTGCAGCCAAGAAGGCGGGAGGCTGGAGTAGAGGGAAGCCTGCAACCGGAAGTGAAGGCAGATTTCCCTCC
TTCGTCGCTGTTGCTGCCGCCATACGCGCTCTCCCTGTTTAGGTAAGCTTTGGCCTTCGCTACAATCCGTTTCCATCTGCGCTTCTCCGCACCC
ATCCCGTCACATGGGTTCCTGATACCCTTTTCACAGGCGATGGTCTGGTCGCTGGGGCCTAGTTGGTTCGCTATTTCCTTAGCTTGCATCCCTT
TCGAGAGCAAAGAGCTCCTGGGGGAAGGAAGGGAAGCTAAGGGGGGACCCAATCCAAGATGGTGTCCTCGGCGCCATTGTGTTCGTTTTGCTCC
CTTCTTCCAATGGGTTCTTCTCATATTGGAGGCCTCAGCATCAATGAGAGGCGGTGCTCGGCGTCCCTTGGTCTTGGTATTTGCGGAGGGCGGG
GCTCTTCTCACCTTCCTTGTTCTTTCTTGAGCTCTTTTTCGGCCCTCGGTGGGACTGGGAGGAGGAGCTGGTTTCTGGGCCCAGTTGGATTTTT
CTCACCTTGACTTGCCCAACTTAATTTGGAGTGCCTTCCAAGTGTTTACGATACGATTGGTGTCATTGTATGTTTCTCCAAAAGGAGTCTCACC
TTCGTAGCGTAACAGTGATCTGAGACCACTTGCTAAAGATCCTGTTAAAGCCTGGGCGGGGATTGCCTTTCTCTGTCACCTATTAGCTTTCTTA
TTGTAGGGTGGAGACATGAATTTTGTTTTTTTGTGGCCGAGCCATTTGTCTTGCACCGCCCCTCCCCCCCATGCTAATTACACAAGGCTTGCTT
AAACAGCGGAAGGGAGGATACTCAGAAGTGGGAGGCTGAGAGCTATGGGAGGTGGACGGCGGCCATATGATGTTTTCTTTTCGAAACGTGAGCG
CTTTGCGCAGTGATGACCCTCATCTATCACCCTTGACTGATGGCTGCTGAGTTAGGCATCCATAACCGTGGGATTATAATAGGGAAAGCGGAGT
CTTCCTTTGAGGACTTTTCAGGACTCTACTTGTCATCTCCATTTTCCACTTTACTAAGTTATTAGTCATATTTTACCTTTTATTATCTATTCTA
TTTCCTCACTGTTACTTTCAGATCAAGAATTTATAAGTTGGTCTTCCCCTTCCAACTTTTCTGGTTTCCGCTACTGTGATTGCTAATCTTGTTG
GGAACCTCTGTCCTAACCACTTTCCCTGGTACTGCTTTTTCTGTTCTGTTATATTTGCTTTTCGTTTTTATGTTTTGTATCTGTTTTTCTTTCC
AGGTAAAAGTTTCCTGGTTTAGGGAAAGTGGGAACTGGGGATGGAAAAATGGTGAAGGCTGTGCTCGTGATTAAGTCTTGCTTTTTTTTTCCCC
CCTCCAGCTCTTCTGTTAGAAATAGTATCTTTGTTTTCCTTTGCTGTTCCTCAATCCCCTACTCTTCACCCCTTGTTTTCACCTATTTTGCGAG
AACCCATCCAGATCCCCCTTCCCTTCTTCCCCTGCCGCCCCAGTTATGGCAGAGAACGATGTGGACAATGAGCTCTTGGACTATGAAGATGATG
AGGTGGAGACAGCAGCTCGGGGAGATGGGGCTGAGGCCCCTGCCAAGAAGGATGTCAAGGGCTCCTATGTCTCCATCCACAGCTCTGGCTTTCG
TGACTTCCTGCTCAAGCCAGAGTTGCTCCGGGCCATTGTCGACTCTGGCTTTGAGCATCCGTCAGAAGGTAAATTTTCTCTTGGGCATGTAGTG
CTCATTGGGCTCTTTAAGGGTACAATACAAAGATGTGTTTGTCGTTGCTCAGGTGGTGGTAAGCGTTTATACTTAAGGCTAGATCAGGGCCAGG
TGCAGTGGCTCACGCCTGTAATCCCAGCACTTTGGGAGGCCGAGGCAGGAGGGTCGCCACTTGAGCTCAAAAGTGCAACAGAAGCCTGGGCAAC
ATAGCGAGACTCCTGTCTCTACAAAACGTTCAGAAATTAAGCAGGTGAAGGTTGAGGCTTCAGTGAGCCGTGATTGCACCACTGTGCACCAGCC
GGGGCGACAGTGAGGAAGAAAAAATCGGGATAAGTATCAAAAACAATTTTGGATAGAGGAGGCTTATACAGGCTTATTCTTTCTTTCGTGATAG
CACCAAAGTGCTAATGATCCAAAAGTGACTTCCAGGTCTGCCATTCATTCTTGTGACTGGCTTTTCTTGTCTGCTTATTTTTAATTTTGTCACT
TGACTTCTAATTTTAAATTTCCAGAAAGGTCCTGCTTGGACCTGTAGTCTCCCTCTGTTGGGCCAGGCCAACTGTGGTCTCTGGAAACCTCTAT
GACTGGTTTAGAGATGACTGGCTTCTGGGTCAGGTACCAAGTCCTTCATTTTGTCCAGGGTTGTAGTAGTTACGTGACCCGAAGTATAGCAACC
TAAGCAGGAGAAGTGGTCTGTGGCAGGTATTCAAATGTCATGAATTGTTACAGATTAAGAAAAATAAGGACAGAGCTAGGATCATTGAAGGTGA
GCGGTTGGTAGATGCAAGGGGTTTGTTACTAGGACTGGGAAGGCCTAGATCTGGAGGAGGCTAAAGCTAGGAGGAATTAGGAGAGTCTGATTTT
GAGGTGAATGTAATTGAGCAGAGAGAGGTAAAATGGGTCTGGAAGTTGGCAAGAACCAGGTAAATACTAGACTTTGAGAATTGAGTGGTAAGAA
ATGGGCTTGGCATGGTGAAAAAGGTAGAGTTATCTGGAGACTGAAGTCTAATTTATCTTCCTCCCCCCCCAACTTTTAGTCCAGCATGAGTGCA
TCCCTCAGGCCATTCTGGGAATGGATGTCCTGTGCCAGGCCAAGTCGGGCATGGGAAAGACAGCACTGTTTGTCTTGGCCACACTGCAACAGCT
GGAGCCAGTTACTGGGCAGGTATATTTGGGGAGAGTGCTGGGGAGGGGATTTTGGTTAGGACTATAAGGGAAGGGTGTTTTTGTCCTAACTACA
TGATGCTTGCAGAGCCATGAGCACATGACCTCTGTTACCCTTGACAACCTGACAGCTGTGGGGGATGTTCTGTCGCAAGCGTGGGGTTCATGAT
TTAGATCACAGAATTGAAGTCATTTATTATCGGCCCAGGTGTGTTTTTGTGACAGTCACTTCCCTAGAGGGGATAATGAAGAGCTACATTTACC
ATATGTCTCCGTATACTTCCTGCCTAACGTGTCTGTGCTGGTGATGTGTCACACTCGGGAGTTGGCTTTTCAGATCAGCAAGGAATATGAGCGC
TTCTCTAAATACATGCCCAATGTCAAGGTAAGCCAAGGTAAAGAGACCTGAGAGTGAGGGTGTGGCAAGTTGGAGGGATAAGAAACTTGTAGGC
CAATAGTCTCTTTAATTTTGGAGAAGCTTTAGTTTGCTGTGGTGTAACAGAGTGTTGAGTTCCTATGTAACAGCAGGATTCGTAATTGGGCTAT
GGATGATGCTTAACACAACACCACCCTTTTCTTACTACTTTATACTGACTTTGAATCATATCAGTTTAATAATTTTGGGGTATGTGGCAGAGAA
AGCCGGAAACTTTAAAACAGCTCCAGTGGTGTGTGAATATTGAGGATTCTGGCCAAGTGCACAATGGCTTACACCTGTAATCTCAGCAGTTTGG
GAGGCCAAGGCGTGTGGATTATTTGAGGTCAGGAGTTTAAGACCAATGTGGCCAACAGGATGAAACCCTCTCTCTACTAAAAATGCAAAAATTA
GCCGTGCATGGTGGCACACACCTATAGTCCCACCTGCTTGGGAGGCTGAGGCAGGGGAATCGCTTGAACCCAGGAAGCAGAGGTTGTAGTGAGC
TGAGATTGTGCCATTACACTCCAGCCTGGGTGGCAGAGTGAGACTCCCATCTCAAAAAAAAAGAAAAAATCTGATTGAAGTTAAGCATTTTTGG
CAAGAATCCTTCATAGGTGATGCTGTATCTCCTGTTATGCCACAAATCTGGTCGACTTATGTTAGTTATTTTATTTTATTTTTATTTATTTGTT
TTGAGATGGAGTCTCGCTGTGTCCTTCAGGCTGTGAGTGTAGTGGCGCGATCTCAGCTCACTGCAACCTGCGCCTCCCACGTTCAAGCGATTCT
CCCGCCTCAACCCCCCGAGTAGCTGGGACTACAGTGTGCCATCATGCCTGGCTAATTTTTGTTTTTTTTTAGTAGTGACAGGGTTTCGCCATGT
TGGCCAGGCTGGTCTCGAACTCCTGACCTCAAGTGATCCACCCACCTCGGCCTCCCAAAGTGCTGGGATTACAGGAGTGAGCCACTGCACCTGG
CCTCATTAATGATTTTAGATTTACCATAGGATTAGCGTCGTGACAGTCTGATTCCACAGTTGTTCTTTTCCCCCTTGAAACCAGAAAGTAGTTT
CTGGTGTTATTTGATACTGTACCAAGGCCCAGATCCCCAAACAACTATTCACCTAATGGTTTTAACATGAAATGATAATATTTAGCCCGAAGCA
GTAATTTCATGGGGTTTGTGTGAAAAGAGTTTGAGATTCTGGGTTTATTTAGGAAACCTTAATGTTCCATGTGTTTTTTGTGGTACTTTACACT
AATCTGGTGATTTCTTGCTGTCCTTTATTTATTTATTTTTTATTTTTTGAGATGGAGTCTCGCTCTGTCATCCAGGCTGGAGTACAGTGGCTCA
ATCTCGCCTCACTGCAATCTCCACCTCCCGGGTTCAAGCGATTCTCCTGCCTCAGCCTCCTGGCTAAATTTTGTATTTTTTGTTTTTTAGTAGT
AAATTTGTAAATTTTGTATTTTAGTAGAGATGGGGTTTCACCGTGTTGGCCAGGCTGGTCTTGAACTCCTGGCTTCAAGTGATCCACTTTCCTT
GACCTCCCAAACTGCTGGGATTACAGGCGTGAGCCACTGGGCCTGGCTTTATTTTATTTTTATTTATTTTATTTCTTTTTGAGATGGAGTATCA
CTCTTGTTGCCCAGGCTGGAGTACAACGGTGGGATCTTGGCTCACCACAACCTCTGCCTCCCAGGTTCTCGTGCCTCAGCCTCCTGAGTAGCTG
GAATTATAGGCGTGTGCCACCACACCTGGCTCCTTTATTTTTTAAATGAAGCCTGGCCTCATAAATGAAGGAAGTTGGTTAGATTAAGTCAGTA
GAACTGAATTATTGTCCTGACTGCTCCGACTAGCCATGTAACTTTAGGCAATCACCCTCAGTGTTTGGTGGGGGGGACATAAAATTTTTAAATT
AGGTGACCTCTAAAGTTAGTTTTAGTTTGGAAACATGCTAAAAATTGGTTTAGCTCAAACAGAGTGGGAACCCTGGGGGGATTGGACTCTTTCC
TTCCTCTGTTTTGAGACTCTTTGCTTCTGGCTCGGCAGGTTGCTGTTTTTTTTGGTGGTCTGTCTATCAAGAAGGATGAAGAGGTGCTGAAGAA
GAACTGCCCGCATATCGTCGTGGGGACTCCAGGCCGTATCCTAGCCCTGGCTCGAAATAAGAGCCTCAACCTCAAACACATTAAACACTTTATT
TTGGATGAATGTGATAAGATGCTTGAACAGCTCGGTGAGTGGCAGTGCTGGGGCTTGGCTAATGCTGGGGAGTTGTTCTTTGGAGCCAAATGAT
GTTTATTTGAAACAGGAGCACCTCAGTGCAAGGACGACTCTTATCTATCACCCATGACTGATGGCTCTGGGTTCCCTGGTTGGTCTTTATTATG
CTTTTAAGCACAGTAAAGGGTGTCATCTATCATCTTTCTATGATTTTTGTTTTTAACCTTTGAGAATAGGGGACTTTGATAATTTTAGGCATAA
GTCATCACCACCACCACCGTTTTCATTATAGATTCATATACTGGGAGTCATAGGGGAGATTCTAAACTGAAAGAGAAGACAGTACCCTTCTGGC
ATCTCCAGCACAGCATTTACAGTCAGAATTTATAGCTGAATAAGTGTCTAGACTCAGGTCTGGGATTAATGTAGAGAGTGTTTGTAGCAGTTTG
TGTGATGTGGTATTCTAGTGTGCCAGGTGGGGTTAATGGAAGATTTTTCTGTAAGAATTGAATCTTGGTGAATGAGAGTGGGGTTGGACATAGG
CCCCATAAGTCATTACAAATGATCTTTGGCAATTCTATATGGTGAGCTATAAAGGTGGGCTCCAGGTAGGGATGTCATATTTGCCTGACTTGAT
AGAAAAGTAATCCAGAGAGTCATAGATGGACTCTGATATCTGGAATATAATATGTGCTTCATATTTGTAGTCTGCTGAAGGCTGGCTGGGGCTT
GGGCAGGAAAGGGTTGGGAGAAGGTCCCATAAAGCATGTTTTGAAGGCCTTGAGAGCCTCTGCACTGGGCTTTATCCCCATTTCATAGTTGGGA
ACTTTGGGGTTTTACCTTATTTCTTGCTTGGTTAAAACAAACAGCTGGAATCTGATCCCACTTCTTGATTCCAAGTCCATTGCTCTTTCCATTG
TGTTGTTACTATTTCCAGCAATCTTCACCTCACTGGGAAGTCTACCTCTAATCTTTGTTTATCATACCTGCTTATTTTCTCCTACAATTTTTTT
CCTTGTTCTTGTAGACATGCGTCGGGATGTCCAGGAAATTTTTCGCATGACCCCCCACGAGAAGCAGGTCATGATGTTCAGTGCTACCTTGAGC
AAAGAGATCCGTCCAGTCTGCCGCAAGTTCATGCAAGATGTAAATACCCTTCTACCTTCTCTCCCTCCACTCCCCGCCCGCTGCCTCCTCCCCT
TCCTCGCCCTCTTCCTCAGACTCCCTTGTCATTCAAGTGCCAAGAAGGCGGCTTGTGCCCAACTGGGAGTAATGACTCCTTGAAGAGACATACA
GAAGCAGAGACAGCTAGTGTTAGGGCCTGCGCGGGTGCCAGGGAAACTCCGGAAGACTTGGTCGGGTTAATGTGAGAGCGGGTAGTGTTCGACT
TTTTCATAAATCACAACATTTTTGAACCTCTTCTCCCTTCCGGGGAGGGCAGGATTTTTCTCCCCTACCACCCACCCATCCATCGTCTCTTACA
TGCACCCTACAGCCACGCACCCTCAAGGTGGCATCGAGCATACAGCTGGAGCCTTCTGCTCACCAAAACTCCTACTTCCCGGTGGCAGGAGAGC
AAGAGAGGGACAGACAGATGGCAGGGCATGTCCAAAAGAAGAGCATCAGCACAAATGAATCCTCCCCTTCCCCACCTCCAGGGGTGGGGGCCTT
TGGCACCTCAATCCCCGATACCCTACTCCTTCCCACCCACATCTCCTTGCACCCATCTGGAACCTCGGTTGATGTGAGCCGGCAACAGAGAAGC
ACCGTGGCGCGGCGAGGGAATGCAGACGGCACCCAGCGGTGGATGGCGGCAGCGGAGGCCGCGGGGAAACCTGACCACGAAGCTGAGGACCAAA
CCAGCCTCTTTTTCCGTTCCCGGTTTTTTTCCTGAACCCAACGCGTGCCGTGCCCCGTTTCCCCCAATATGTGTTGGGGAGGGGTGTCCTGAAT
GGGGTGGTAGATTTTTTTTCTTAAAAAAATTTTTTTGTTTTTTTTAATACTCAGAGGAGAGGGACATAGGAAAGGTAAAGTGGATGTAATCGGG
TGGTTGTTAGGGTTTGGGGCTAGGTGGGGCCAATTGCATAAGCAGTGGAGTGTGTTCTTCCCCTCCCTGCAGTGTTCCTTCCCGTGGGATGATC
ACTCTTTAGCTGTATTTGGGGCTAGAATGAGATTTGAAGGAGGCCATGGAACTTCTCTTTAGAAAGCCTGCCTTGGCTGGGCCTGGTGGCTCAC
CTCTAATCCCAGCACTTTGGGAGGCCAAGGTGGGAGGATTGCTTGAGCCCAGGAATTTGAGACTAGCTGGCGCAGTGTAGTGAGACTTTGTCTC
TACCAGAAAAACCGGGCGTGGTGGCGCATGCCTGTAGTCCCAGCTACTTGGGAAGCTGAGGCAGGAGGGTTTGCTTGAGCCCGGGACGTGGAGG
TGGCAGTAAGCTGTAATTGTGCCACTGTACTCCAGCCTGGGTGATAGAGTGAGACCCTGTATCAAAACAAAACAAAAAACAAAACCTGCCTTCT
GGGATTGGGCTTCTGGTTTTTTTCCCATGACACACACATCCTTTCCTATTTTGTCCTCTGGGTCTTCATATTAACTATCTTCCCCCAGGATAGT
ATAAAAAGTGTTAGGAAAGTTGGGCTTTGGAGTTGTGGTAATTTCTGTCTTTGTTACTTTCCTCCCCTTCAGGGGGTTTTTTAATTTTAAAGAT
GAATGCAGTGAGGTATAATGGTGTGTGCCTGTAGTCTCAGCTATTCAGGAGACTGAAGCAGGAGGATCACTTGAGCCCAGGAATTTGAGGCTAT
AGTGTGCTATGATTGTGCCAGTGAATAGCCACTGCACTCCAGCCTGGGCAACATGGTGAGATCCTGTCCCTTAAAAGCGTATCTGCTGCTCTGA
ATTTGGTATTTTAACACCACTTACTGATACCTTTCCTGTAAACCTGTAGATGGTTTAATTCTTAGTCAAGAGACCAGTCTCATCTAAAACTATC
CTGTTGTGGTCTGACGGCAAGTAACTCATCTTGAGTAATTTTTGTTTCTCCTTAAGTGGCATTTTGACTGTCCATTGCAGCATTCTGATCTTAA
AAGACATCCACTTTGCTAATGCACACGAGATTCTCTTAGTTGAAGTAGGAGAATCAAATGGAGCAGTTGTCCTCCCCCCACCCCATGTTCTTAG
AAGCACCTCTGATGGAGTTATTCTGACCTTGAGTCACTGCCTCCCATCATTTCCCAGATGTTTGGTCCTTGCTCTCCCTTTGAGAATCATCTCC
CATTTTCTTTCCTCTCCCACCTCTATTTGAGGTAATCGCATCTGTGCCATTGGGTGGTTTCACTGCTCCTTGACTTCATTTGCAGTTTCTTTCC
CATGATAGTTTTTAGTTGGGCAGTCTTAAAACTCATCTGATAGGAAGGAAATTAGATGTAATGTGAGAGAGACCACAGTAAAATGTGGGTATTT
TTGCGAGTGGGGTGGGGTTTTCAATCTTCTCTTTCCTCCCCATCCCCCCATGGGGTGTATTGGAGATCAACTTCCTCCACCCCCCCAGGTTTAA
CCCCCCCACTCTGCCCTCCTCCCGTTCCCCACCCCCTTCCTCCCCCCCAGCCAATGGAGATCTTCGTGGATGATGAGACGAAGTTGACGCTGCA
TGGGTTGCAGCAGTACTACGTGAAACTGAAGGACAACGAGAAGAACCGGAAGCTCTTTGACCTTCTGGATGTCCTTGAGTTCAACCAGGTCAGT
TAGACGTCCAGTAGGGGGATGAGCATTGGAGCACTCCAGCTGTAGCAGAAACCTGGATATTAAGTACACTTTTATTGAGGAAATCACATGTGTG
ATGTGGGAGAGAATAATGAGGGTATAAATATCTTAGGGGCTGAGCATGAGTAAGGTCGGAGCTGCTTTTCTATTCTATGGCTGGCACGGGTATG
TCCTCAATAACCTCAAGGAAAATAAACTTCAAAAATTAAGATCCTTGGCCAGGCACGGTGGCTTATGTGTGTAATCCCAGCACTTGGGGACGCT
GAGGGAGGTGGATCACTTGAGTCCAGGAGTTTGAGACCAGTCTGGGCAACATGGCGAAACTTCATCACTACCAAAGAAGAAAAAAATTAGCCAG
GTGTGGTGGTGTATGCCTGTAGTCCCAGATACTCTGGTGGCTGAGGTGAGAGGATAGCTTGAGCCCACGAAATTGAGGCTGCAGTGAACTATGA
TTGCACTACTGTGCTCCAGCTTGGGCAACAGAGTGAGATCTTGTCTCCAAAAGTCCTTGAAGGATTTTAGGAAGTTGTTAAAAGTCTTGAAACG
ATGTTTGGGGGCATGTTAGGGTTCTTGAATGTTTAATTCCTCTAATAACTGCTTATTCAAGAGAAGCATTTCTGACTGGGTGCAGGGCAGTGGC
TCATGCCCATAATCCCAGTACTTTGGGAGGCTGAGGCAGGAGCATTGCTTGAGCCCAGGACTTCAAGACCAGCCTGGGTAACATAGGGAGACGC
CCGTCTCTACAAATAGTAAAAATAAAAAATAAAAAAAGTAGCTGGGCGTGGTGGTGTGCACCTGTGGTCCCAGCTGCTTGGGATGCTGAGGTGG
AAGGATCTCTTAAACCCAGGAGGGTGGAGGCTGCAGTGACTTGCGATTGCACCACTGCACTCCAGTCTGGGGGACAGAGTGAGACCCCATCTCA
AAAAAGTGTTTAATTAATATACTTGTGAGTGGTCTATTTGCATTTAAAACTGCTTTCTAGAATTAGGATAGCTCCCTTAGGTTTAATGTTTTGG
TGAGCAGGAATATCAGTTACCCCTCCAGATCTTAATTCTAGTTTTTTATCACTTTTTCATGAGGTGATCTCATCCTCATCTCCTAGCATGTCTG
GCAATTTTGATTTCTGAACTCTGTGCTACCTCAGAGGCCAGCTTCCTTAGGGAAAAATCAGTGCTGAAATAAAGTTATATTTCCTTTTCTGCTC
TAAATATATAGTGGGGGAATAAGAGAAATGAAGAGGAATTCCTGAGAACGTAATTACTAGAAACTCCCCTCTCCCACGTAATGTCTCTCACACA
CCATGGACCCCTATTCCCCCAATTTGCGACCCCCCACCCCACCCCACAACAGGTGGTGATCTTTGTGAAGTCTGTGCAGCGGTGCATTGCCTTG
GCCCAGCTACTAGTGGAGCAGAACTTCCCAGCCATTGCCATCCACCGTGGGATGCCCCAGGAGGAGAGGTGAGCTGAAGATGGGAAAGATATTT
TGTGTCCTTGGGAGAAAAAGACAGTTGAGAGAAGGGAATCTCAACATGTTTTAAATTTCCTTTCTCACAAAGGCTTTCTCGGTATCAGCAGTTT
AAAGATTTTCAACGACGAATTCTTGTGGCTACCAACCTATTTGGCCGAGGCATGGACATCGAGCGGGTGAACATTGCTTTTAATTATGACATGC
CTGAGGATTCTGACACCTACCTGCATCGGGTAAACCTCACAGGCTGAAAAAATCCCACTCTCCCATTCCCTTGTTTTCTGTTTGTACATCTTCA
TTCCTGCCTCTGGGTCTCTTTCCTCTTCGGTCTTCCAGTGCTACCCTCTGTCTCCCTCCAGGTGGCCAGAGCAGGCCGGTTTGGCACCAAGGGC
TTGGCTATCACATTTGTGTCCCATGAGAATGATGCCAAGATCCTCAATGATGTGCAGGATCGCTTTGAGGTCAATATTAGTGAGCTGCCTGATG
AGATAGACATCTCCTCCTACAGTGAGTACTGATCTCATGAAACCCTTTAGGTCCTCCCTGTTCCTTAGTGTGTTTGTCCTAAATCCCATCACAT
AGGTCATGGGCATCTGATGCATAATGGACACTTGACTGGTTCATGCCCCCTGGTCTTTGATGCTGTGTTGGGATGTTTTTCTGACCTTTATGTG
GGGTTTCTGTCTTCTCTCATCATATTACATCCCTTCCCTCACCCCCACGTCCGTCCTCTGAACCCAGGCAGTACACCAGTGTCTGCATGTGTGC
CCTGTGTTCCTGCCTCACTTTCCCCTTTTCATGCCTTATTCTGACCATGCTACGTTTTCTTCTCAGTTGAACAGACACGGTAGAAGACTCGCCC
ATTTTGGAATGTGACCGTCTGTCCTTCAGGAGAGGACACCAGGGTGGGGGTGAAGGAGACACTACTGCCCCCACCCCTCACAGCCCCCACCCCA
TGGCTTCCATCTTTTGCATCACCACCACTCCTGAACCCCCATTTCTGATTTGTCAGAATTTTTTTTTAACAAAACTAAAAATGAAACACATGTG
TCTGTGGTATCTATAAGTGCTTCGTCCCTTTATTGTATTTGGGGTGAGGTTATTTTAGGGCATGGTCCAGGGTGAATTCCTATAAGGCCTGGGT
GCCCTGCCTGCTGTGAGATCAAAGGGGAATGGGACTAAGACTGCAGAGCCCTGGCTCCCCCACTGCCTGCCAATTGCCTGCGGTTTGTGGTCTC
TTCCACTTTCTCTGGCCTGGGAAGACGCTGGGGTGTTTATGATCCCAAGGCTCCTGGTGGGTGGTGCGTGTATTTTCAGTGCCGGAGGGTGCTG
TGGGCACTGGGGGAAACTTAGGCACCTCCTCCAAGGCTCTCTTGGTGCCTCCTCATCTGTTCCTTCAGCTTCTGGATCTTGAGCACCAGGGCTT
GGGCCTCCCAGGCTCCCTCCTGCCCTTCAAGGAGGGCCTGGTACAGCTCCAGCTGCTGCTCCAACAACTCTTCAGCTCGGGCCAGCTCAGCTGT
GCGGTGGGTCCCAGGGCCCTGGTCAGGGAATTAAGGGAGGGAGCATCAGCCAGGGCAGGGGGCCGAGGCCCTGGGAAGCTTTGTCGCAGGCTGT
GGCTGGAAGTGAGAAATTCCACCTTCCCTATTCGTTTTTGAACCGGTCATTTAAGGACACCTGTACTGAGAAGGCCAGGTAGCTTCCTGTCTTG
GGCATAGGCCTCTGGGTGGTAGTAGGGGGAGCAAATAGAGTTCCCTGGCCCAGGGGCTGTAACTGGCTTCCTTGAACAAGGATATGGGGTCACT
GGAAAGAGGATCAGCCGCTCCTCCCCGCTAAGAAATAATTAACTGTTAGGTGAGGGGGAATAGATCCTGTTCAAGGACTTTGTGGACTCTGCTG
TTGTGGGTGGGGGTGGGGTGAGGGAAGGGAACCCTGAGGTCTGGGCTGGGGGAGGTGGGGAGAGGAGTTGGTAGCTGAACTAAGAAAAGAGCTG
CAGGGGTAGGCATGGTGTGGGGTGGTGCAGGGTGGGATTGAGGGTTTTTTTTTCCCACACCCCAGTGTAAATTCTCACACCCTCTGTTCCTACC
TGTGGTGCCACTTACCCTGGGAGGGGACGTCATCTTCCCATTTCCTCTGGAGTTGGTCTGCTCTTCCATGCTTGCTTTGGGGTTTTGGGAGCAG
CACCCATGGGAGCCCTGGGGTGCCAAGGACCAGGAGGGCAGAAGGAGGCGAAGGAAATGGTACCGAGAGAGCCAGGGCAGAGGGAGGACCATGG
CGGGTGACCTGGCCGGGAGCTGTGTGAGCTGTCCAACGGCCACCAGGAACTGGTTCGCTCCAGGACTTGGCCTCACTTGAGTGCCTGGCCCTGC
CCAGGCCCCAGCCCCCAGCCCTGCCCCTGCCCCTGCCCCACTCTGCCCCACGTCTCTCCCAGCCTGGCCCCAGACAGAGTCCAGGAACAACTCC
TGTTCCTGATGTGAAAAATGTCCCTGCCAGTTTAGGCAGAACTTGCTTTAGAGCACTGGTGCCCAGCCTACCACAGGTCTGTGATTTTTTTTTT
TTGATCTAGTGTTTATTAGGTATGAATTTTACAAACATTAGCGGTAGCTGTGGAGCTGGAGAGTATTGCACCTTCTCCAAGCTGCATGGCGAGA
ACCACCAATAGTGTGGTAGAACTTACAGCCCTTTCCAAGGCCGTGGCTCTCTTGGCCTCCAGATAGCCTACGCATCTCCCTATCCTTGTTGTGG
ACCACTGGGTGTCAGGTTTCCTTCTGATAGTGTTATGGAATGGATCAACTAGGATAGCCTCAAAAACTTTGTATGTGGAATCTTTAACCAGCCC
AGTGAGAATTCAGGACTCAGAGCCCCACAGTGGCATCCAGCTTGCTCTTCTGTAACAGACTGAAGGCTTTAAGCAAACTAGCTGGTTAACACCA
TGATAGACAGGCTTGCTGTTAAGTTGCCTTCTTAGGAACTAGATTTTCAGCCACCGTGGCGCTTATATGTAACATAACCTTGCTTGGCTGTAGC
CCAGTAGCGTGCTTTATTGGGCTGGGTGGGGCGGGGATCCCTGTGGAGAGCAGAGAGCTGGTGGTACTGCCAGCAGCGGACCCTCAGAAGAAAG
CTCATGACATCAGACTGCTTCTTCCATAGCTTCTGGATGTACTTGTATGCACCATCTTGGTTTACCCAGTGGCTGCTGCCAGACAGAAAGGAAA
GGCTACCACAGGTCTTGTGTCTTTTTTTTTTTTTTTTTTTGAGACGGAGTCTCGCTCTGTTGCCCAGGCTGGAGTGCAGTGGCGCAATCTGGGC
TCACTGCAAGCTCTGCCTCCCGGGTTCACGCCATTCTCCTGCCTCACCCTCCCGAGTAGCTGGGACTACAGGTGCTCGCCACTACCCCCGGCTA
ATTTTTTGTATTTTTAGTGGAGACGGGGTTTCACCGTATTAGCCAGGGTGGTCTCGATCTCCTGACCTCGTGATCCGCCCGTCTTGGCCTCCCA
AAGTGCTGGGATTACAGGCGTGAGCCACCGCACCCGGCATGCCTTTTTCTTAACACTGTTTTCTCACTTCACTCTGCAAGGTAGGAATTACCTC
ACTGGTTTGCACCTGAGCAAACTGGCTCAGATGGTTTCATTCAGCATTCACTGGGGAAGTGTCTGTTGGGGGCAGCTCTAGGCTGGATGTGCTC
GAAGGTCCACAGCTGGTTGTTGGTAGGGCCTGGAGGGTTCATGTCTAGGTCCACCTGACTTGAAAGCTCATCCTGACCTTGCTTAAGTGCTGAT
TCCCCTTTCCAGATGTACCTTTTATTGTGCTTCCCTTTATTGCTCTTTGCAGATGCTGTTTTTTATTTAGAGATTGGAGGCTTGTGGCAACCCT
GTGTCAAGCACATCAAACAGGTCTATTGGTGCTATTTTCCCAACAGCAGGCAGACATCATGTCTCCATGTCACGCTGTGGAATTCTCAAAATGT
TTCAAGCGTTTTCATTATTATACTTGTTACAGTGACCTGTAATCAGTTACTGAAGTTAACTATTGTGATTGTTTTGGGACACCATGAGCGATGC
TCATATAAGACAGCAAACTTAATCGAAAAATGTGTGTGTTGTGACTGCTTCACCAACTGGCCATTCTCCCGACTCTGCTTTCCACGCCTCCCTA
TTCCCTGAGGCACAACAATATTGAAAGGAATAATCCATGCGGCAAATGGCAAACATCATTGTCTTATTTTAAGAAGTTGTCAAAGCAGCCTTCA
GCAGCCATGCCCCTGATCCATGGAGGCAAGACCCTCCCCCAGCAAAAAGATCAGGATTAGCTGAAGCCTCATATGATTGTTAGCATTTGTTTAG
CAATTAAGTATTTTAAAATTAAAGTATATGGCCAGATACAGTGGCTCACGCCTGTAATCCCAGCACTTTGGGAGGCCGAGGTGGGTGGATCACT
TGAGGTCAGGAGTTCAAGATTAGCCTGGCCAACATGGTGAAACCTCATCTCTACTAAAAATACAAAAATTAGCTGGGCATGGTGATGGGCACCT
GTAGTCCCAGCTACTTGGGAGGCTGAGCAGGAGAATGGCTTGAACTCAGGAGGCGGAGGTTGCAGTGAGCCAAAATCGTGGCACTGCACTCCAG
CCTGGGTGACAGAGCGAGACTCCATTAAGTATATACACAGTTTTTTGTACACAATGCTACTGTACACTTAACAGACTACAATATAGTACAAACA
TAACTTTTATGCACAATAGGAAACTAAAAAGTTTGTGTGACTCACTTTGTTGCTATGGTCTGGAAACAAATCTTCAGTATCTCCGAGGTATGCC
TGTCATTTCCCTTTCCCTCTTCTTGCTGGCCCAGAATGACCTTGTTTCTTGCCCCTGTCTAGCCCTGCATGCTGTAGGGGTTTGCCTTCTCTGG
TAGGTCTGGGCACTTTGTATCCCTTGTAACCTTGGCTCCTGGGATATGACACTGGTACAACTGGCCTCAAGTTCTGTTGGACTAGTGAGCCTCC
CCCAACACCTCCTGAAGTAGAACCAAAGGCCTGTGCACACACCGTGCATGTGTGAGTCTGCATAGAGATGTCAGCTTCCTGCAGGGTGTTCTGA
AGGGATGTCCTGTTGTGACTGGACTGTGACATAGCCACAGGCCCAGAGGCAGGAGTGGCTCAGAAGGGAGTGGCTGCTCCCAATTTTGATCATC
TAGGAACAGGAAGGTCCTTAGAAAACCATGCCCCAGAAGGCAGGATTGCTGGAGAGTGGACAGCTGCTAGCCAGCTCGCTATCTGGATATCACT
CTGCATTGGGAGGGAAGATGGCCTCTGCCATGGTGTAAGAGTCCAGGAACCAGGCAGTGAGGACTTCCCAGCGGTCAGTGCTTCTCACACTTGC
GGGCCAAAGCACCTTTAGATGAGGCCAAAGACTTTACGTTCCTCATTAGCTGACTTTTTCCCACTTAAGTGGAAAAAGAACCCAGAACCTTTGT
AAAAGTTTTAGGGGAGAAGGGCTTTCCCTCTTGTATCTTGGTGATAAGGTTATGCATGACTCATACTTTAATTGCAATGTGTACACAGCTAAAG
TCTTAATTATTAGAATATAAGAGCCCCAAACTACTGTTATTATAGATAAGCGAAACTATGCAGTATATGGTTAAACAATCCACAACTAATTAAC
ATTGAAAGTTGGCCGGGCGCAGTGGCTCATGCTTGTAATCCCGGCACTTTGGGAGGCCGAGGCAGGGGGATCACTTAAGGTCAGGAGTTCAAGA
CTAGCCTGGCGAACATGATGAAACCCCGTCTCTACTAAAAATACAAAAAATTAGCTAAGCGTGGTGGTAGCCACCTGTAATCCCAGCTACTTGT
GAGGCTGAGGCAGGAGAATTGCTTGAACCTGGGAGGCGGAGGTTGCAGTGAGCTGAGATTGTGCCACTGCACTCCAGCCTGGGCGACAGAGCAA
GACTCCGTCTCTCAAAAAAAAAAAAAAAAAAAAAAAAGAAAAGAAAAAAAATTAACTACATTTTTGGGAGGTGGACAGAGCAATGCTCTGTCAC
CCAGGCTGGAGTGCAATGGCACAATCTCTGCTTGCTGGAACCTCCGATTGCCGGGTTCAAGCAATTCTTATGCCTCTGCCTCCCAAGAAGCTGG
GATTACAGACGTGTGCCACTATGCCGAGCTAATTTTTGTATTTTTAGTACAGACAGGGTTTCACCATGTTGGCCAGGCTGGTCTTGAACACCTG
GCCTCAAGTGATCCGACTGCCTCAGCCTCCCAGAGTGCTGGGATTACAGGTGTGAACCACCGTGCCTGGCCCTCTATCTGTTAATTTAAAAGAT
TAGCAGCCATTTAGAAAAAACAACAAATGAGACTTTTGCAAGACAATCTAAATGATACACTAATAACAATCCTTTCCGAAAGTGACATTTCAAC
CATGTGAGTTTCTGCTTTAGGTTATGAACTCCAAAATGGACTAAATGGACTAACCCCCAATAATTTATAGTAGCTAGTTTTTTTTTTTTTCACA
GTAGGTAATTCTAAACCATAAATAAAATAGAATCTGAATTTTGGCTTTGTTCACCTGTGGGAACTTAATTAAGAAAGCACTGGCCTTTGGGTCG
GTTCAAATATAGTGGATGAGGCCAGGCGCAGTGGCTCACACCTGTAATCTCAGCACTTTGGGAGGCCGAGGCGGGCGGATCATGAGGTCAAGAG
ATCGAGACCATCCTGGCCAACATGGTGAAACCCCGTCTCTACTAAAAATACAAAAATTAGCCAGGCATGGTGGTGCACGCCTGTAGTCCCAGCC
ACTCGGGAGGCTGAGGCGGGAGAATCACTTGAACCCGGGAGCCAGAGGTTACAGTGAGCTGAGATCATGCCACTGCACTCCAGCCTGGCGACAG
AGCGAGACTCTTGTCTCAAAAAACAAAAAACAAAACCAAAAAGAAAGAAAACCAAATATAGTGGATAATCGTGGATCTCATAATTGTAGAAATG
AAGGAATTAAGCTAAAAAAATACATAAACCAGAATACCTAGTGCTAAAGTTGAATGTCCCCACCAAAACTCGTGTTGACATTTAATTGCTATCC
TAATGGTATTAAGATGCAAGACTTTTTTTTTTCTTTTTTTGAGATGGAGTTTCGCTCTTGTTGCCCAGGCTGGAGTGCAATGGCACAATCTCAG
CTCACTGCAACCTCCGCCTCCCATGTTCAAGTGATTCTCCTCTCTCACCCTCCTGAGTAGCTGGAATTACAGGCACATGCCACCACGCCCAGCT
AATTTTTGTATTTTTAGTAGAGACGGGGTTTCATCATATTGGTCAGGCTGGTCTTGAACTCCTGACCTCAGGCGATCCACCTGCCTTGGCCTCC
CAAAGTGCTGGGATTACAGGCATGAGCCACCGTGCCCAGCCGATGTGGGACCTTTCAGGGTTGATTAGATTGAATAGATTAATGCCATTGTATG
GCATGATAGAAATCAGTTCAGCCTCTTTGCCCTTCCACCTCTCACTATGGGATGATACTGCAGCCAGGCCCTCATAAGATGCCAGTGTCATGCT
CTTGGACTTCTCAGCCTCCAGCTCTGTGGGAAAAACATTTATTTTCTTTATAAATTACCCAGTCTGTGCTGGGTGTGGTGGCTCAAGCCTGTAA
TCCCAGCACTTTGGGAGGCCAAGGTGGGTGGAAGGCTTGAGCCCAGGAGTTTGAGACCAGCCTGGGCAACATGGCAAAACCCATCTCTACAAAA
AAACACAAAAATTAGCTGGTGTGGTCGTGCGGGTCTGTGGTCCCAGTTATTTAGGAGGCTGAGGTGGGAGGATCACTTGAGTCTGGGAGGTGGA
GGTTGCAGTGAGTCGAGATCATGCCACTGCACTCCAGTCTGAGCGACAGAGAGACACCCTGTCTGAAAAAACAACAAAATAAATTACCCACTCT
GTATTATTCTGTTATAGCGGCAGGAAACGGACTAAGACACATAGATTATGTTACTGTGTTTATTTATTTATTGTTGTTTTTGTTATTCCTGACT
CTTAATATAGAGTCTTAATCAGATGAGCATTCTGGCCTGGCCTCCGCAGAAGGGGCCTGTCTTTAGCCACGGACAAGAGGAGATTAAGGCCAGC
ATCATCCACAAGGTCAAGGGGCTGCAGAGCCCCCTAAGGCCAGTGTGCTGATGGGCCCCTCAATATTGTATCCTACCCAGTGGATTGGCAGGAC
TGGGTGACTGACAGGAATCATTGTTGCCTCTATGGGAAAGTCTTATGGAGATGGGGGCTGAGGGATGTTGAAGTTTAGCCATTACATTACAGTG
AGAGAGATTACATTACTAAGTGTCAGAGACCCTTCTGGGCACTTTCTGTTACTGTCACAGGTGGCTTTCACAGTAACCTTTTAAGAGAGCTCTT
TTCATTTTTCTTGTACATGGCTGTCCAGTTGTTCCAGCAGCATTTGCTGAAAAGACTATCTTTATTGTATTGTCTTTGCTCCTTGTATTTATGT
GGGACTATTTCTTGGCTCTCTATTCTGCTCAACCCCATCTCTATTAAAAATACAAAAGTTAGTCGGGCATGGTGGCAGGCTCCTGTAGTCCCGG
CTACTCAGGAGGCTGAGGAAGGAGAATCACTTGAACCCAGGAGGCGGAGGTGGCAGTGAGTCAAGATACTGCCACTGCACTCCAGCCTGGGGAA
CAGAGGGAGACTCCGTCTCAAAAATAAATAAACAAATAAAAATTTAAAAATTAATAAATAAAAATAAAAAAATTAGCTGGGCATGGTGGTGTGT
GCCTGTAATCTCAGCTACTTGGGAGGCTGAAGCAGGAGAATCGCTTGAACCCACGAAGCAGAAGTTATAGTGAGCCGAGATCATGCCACTGCAC
TCCAGCCTGGGCGACAAAGCAAGACTCCGTCTCAAAAGGAAAAGAAAAAGGAAGCTGGAAGCTGAATGAGATGGGCCTTTCAACCAAGGAGTTA
GAAGGCCATCTGGTGGCAGGGCTGGCACAGGACCAGGAGTAAATAAGCCCAGAGAGGACACCAGGGTCTGGGAGTGAAGGCACTGAGCTTGGGT
CCCCCTTTGGAAGACAATGACCTGAGAGCTGTGAGATTTCAGACAAGTTCCCGAACCTTTTGGGCCCTGCTTTCCTCATCTGTAAATGGGATAA
TATCAGTCTCACCAGCTTCTTAAAATTCAATACAATGGAGTTGGGTGTGGTGGCTCACGCCTCTAGTCCCGGCACTTTGGGAAGCCGAGGTGGG
CAGACTGTTTGAACTCAGGAGATGCAGAACAGCCTGGATAACATAGCAAAACAGTCTCTACCAAAAATACAAACAATTAGCTGGGCATGGTGGT
GTGTGCTTGTAGTCCCAGCTACTAGGGAGGCTGAGGTGGGAGGACTGCTTGAGCCCACGAGGTAGAGGCTGCAGTGAGCCATGATTGCACCACT
GCACTCCAGGCTGGGAGACAGAATGAGACCCTGTCTCAAAACAAACAAGCAAACAAACAATAAAGGAAATCCCTACCACACTATCAGGGGCATT
TTGGCTGAGCGCGGTGGCTCACGCCTGTAATCCCAGCACTTTGGGAGGCTAGGCTGGCAGGTCACCTGAGGTCGGGAGTTTGAGACCAGCCTGA
CCAACACGGAGAAACCGTCTCTACCAAAAATACAAAATTAGCCGGGCGTGATGGTGCATGCCTGTAATCCCAGCTACTTGGGAGGCTGAGGCAG
GAGAATCTCTTGAACCCAGGAGGCAGAGGTTGAGGTGGGCTGAAATCGCGCCATTGCACTCTAGCCTGGGCAACAACAGGGAAACTCCATCTCA
AAAAAACAAAACAAAACAAAAAACAAAACTCCCATTTTTGCGAGGCAAATTGGGCTCACAGAGGTAAGCTGCATGTCCCTGTTGATGGCAGAGC
TGGGGTCTGGATGCAGGTCTGCTTCCGGGTAATCCGCTCTTTTGCCTTCCAGGGTCCTGCCTCTTACAATATGAGCTGTCAAGTTAGATGCCTG
CACTCAGTAAACCTACTCTGTTTTAAGTAAAAACAACAAGAAACAAATCTGAATATGCTAGCCTATCTCACCTACGTTAAAGGAAATTTTTAAA
TAGGGGGTTTTTTGACCATTTGGGGGAGTTTTGGGGGAGGGGCCTTCTGTCTATACTTGAGCTGGGGGATGTTAGGGTTGTTCATCTGGATCTA
GAGGTTTTCCTGTAATGTTCTTACTCCAGAAGGAAATCTCTAGATGGGGAAAGAAGGTTTCAGCTTTTATTCTAGTAAGCAGGGCTCTACCTAT
AAAGAGCTGCTTCCACCACTCTTTTTTTTTTTTTTGAGACGGAGTCTTGCTGTGTTGCCCAGGTTGGAATGCAGTAGTGCAATCTCGGCTCACC
ACAACCTCTGTCTTCCGGGTTCAAGTGATTCTCCTGCCTCAGCCTCCCAAGTAGCTGAGACTACAGGTGTGTGCCACCATGCCTGGCTAATTTT
TGTATTTTTAGTACAGATGGGGGTTTCACTATGTTGGTCAGGCTGGTCTCGAACTCCTGACCTCGTGATCTGACTGCCTTGGCCTCCCAAAGTG
CTGGGATTACAGGCATGACCCACCGCACCTGGCCTCCACCACTATTATAATATCACCAGGTTCCCCATTTGAATCCTTCAGTGCCAAAGGTTTT
GCAGAATTCAAATCTTTTTGGGACTTGATAGGGCTGACCTAAAAGTACACTCACTCTATATTAGGTAGCCCAGGAGGGCCTAGGCAGCCCAAGA
ACCAAACACATGAGTGTTTCTGCAGGGAAATGTATGAATATTGACATCAGTAGGATGAAAATAAATAATAGTCTTACTTTAGTTCAGATTAGGT
TTCTGTCACCAAATGAATTTTGGTGGCAGCCTGATGAAAAATGTTGGTTCTCAGAGTGTTTTTGAGTTTAGAATTGTGGTTAAGGGAGTATGGA
CCTGTTGATAACAAAAACAGGAACAAGGCCAGGTGTGGTGGCTCACACCTGTAATCCCAGCACTTTGAGAGGCTGAGGTTGGTGGATCACCTGA
GGTCAGGCATTCGAGATCAGCTTGTCCAACATGGCGAAAACCCATCTCTTCTAAAAATATAAAAATTAGCTGGGCGTGGTGGCATGCGCCTGTA
ATCCTAGCTACTTGGGAGGCTGACGCATGAGAATCACTTGAACCTGGGAGGTAGAGGTTGCAGTGAGCCAGGATCGCACCATTGCATTCCAGCC
TGGGCAAGAAGAGTGAAACTTCATAAAAAACAAAAACAAAAACAAAAAACAGAGAAACAGGAACAACAATCGCCAGCATATACCAAGTGCTTAT
CGTGTGTGCCAGGTACTCTAATTATGTACTATGTCAGTTGATTCTCAAAACATATATGGCACAACATGGGTACTCTGAACATGGGCACAATCAA
TGTACAATGCTATAATGTATAACACAGGACAATGTAGCTGTTAAAAGCATGGACACTCTATCTAGTCCATCTGGGTTATAATCTCTGCTCTACC
AGTGAATAACTGTAACTCTGGCAAATGACTTCTCTATGCCCTGTTTCCTCAGCTGGGAAATGGGGGATAATATCAGTACTCACCTCCTAAGATT
GTTGTGAGGATTAAATGTGTTACTTTATAAGAAATGTCTGGCACATAGCAAAGGTTGTTATTATTTTAATTTTTACACATGGGCAAACTAAGCC
TCAAGTAACCTGTCCAAGAATACGTAGCTATGAAGTGTGGAGCTGGGATTTGGAGCTGGGGTTTGAATCCAGGCAATCTAACTCCAGAGCCTAC
CTTCTATGCTACTTTTTGGCTACGAGCAAACAATCTGTCAAGAAACAAAGTAGCTACTAATCTAAACAGATGTGAAATTTGAAGACCAGTTGAT
CTTTGGGGAATGTTCGGTTCTTCAGACAATGGTAGCTCAGTAATGTAAAGGGACAAATGACAGCCACATGCCAACTTGGTTAACTCCTTCCCTA
GGTCCTGATGACCAAATAACCAGCTTATTTCTCAACTATTGGTTGGCTTTCATTTCAGGTCTGGTCAGCTGCTTATGACCTTGTTCCCCACTGA
GCAGACTCACCATCTGGGCCCTGGCGGGCAGCAGCATGCAGTGCCGTGTCCCCATGGCGGTCCTGGTGCGCAGGGTCACCCCCGAGCCGAAGCA
GCAGGCACAGGGCAGGGGCATCGTGGCGGGCACAGGCCCGGTGCAGTGGTGGGGGCTGCCCAGCATCTACATCGAGGCCTGGGTGTCGCTGGAG
GAGGGCCTGGGCCCGGACCAGCCCTCCTGCAGACAAGTAACGACGAAAGCGACGTTCTCGGCGTTGGCGGCGGGAAGTGGAGGCCATGGAACTC
TTGGGCTGGGGAAGGAAAAAAGGCAGCAGTCAGGACTTCAGCCTTGGCTGGTCCTTCTCCCTCCATCTCTGACATCCCCTGTTGTTTCTCCCTT
TGGTTCCGTCTTTTTTTAATATCTTCAGCAAGAGATGAGGCCTAACCTAACCCTGATCCTTTATCAGATGATAGATTTGAAAAAAAATTTTTTT
TGAGGGGGGTGATAGGATCAGAGGTTTAATTTTTTTAATGTAAAATTCGAGAAAAGGGTAAATAATTGGTTTAAGGCTCAGGAGCCCAGGTAAA
TTTTTAATTTTTAACAAAGAACTTTAAAAAAACCCAACAGGGCCGGATGGGGAAAATTTTTATCAGCAGAAATCTGAGTTTTAAAAAGTCACAG
ATAATCTCCAATAATGATCTAGAAATTGAATATCATGTACCCGGCAGACAGATGTGGAGGCTTCTTCCTCTGGAACCTGGGGGGAGGGGTTACT
CATCAGACCTGCCCCCGCCCCCCCAAGTACCCCCAGAGCCGTAGGCCCAACGCCTGTGTTTAAGAAGCTCGGAGACGGGAGGCGGGAAGGGCGG
AGACACTCCAGGCTGGAGGAAATGGCGCAAGCAGAGACGCAGGTGGAGGACGGAAGTGAACTGTGAGGGGCGTTACCGGATGTCGTTCCGCCCC
GACCGGGTAGTTCTTGGCCAGATCTCCCAGGGGAAACTAGGGAACTTAAATTAAAGGGGCCGTCTGAAACCAGAAGACTGGACTGGAGGCGAGG
AAAAGGAGGCGAGGGGAGGGGAGGGAGAAAAGAGAGTTATTTGGAGGTTTTTTCCCGCCTCCTCTAACTTGGCAGAGAGAGGAGATGGTTCAGT
GATGGACGAAAAGATGAGAAGACAGAGAAAATAGAGGAGATAAAGACAGGATAAAAATCACATTAAACATGGAAAACAAAAACAAAAACCACAG
TGGGACAACAACAGGGACAGATCAAAAAAAAGAAAAAATACAGACAAAAGACGGAAGAAGACTATCGTAGGATGGGGCAAGTGAGATGCAAAAA
TTTGGACTTGAGAAATATGTAGAAAAAGATGGAGATGTTAACAACGGGAGGCAGGGGAGGGGGCGGGATGGTGGAGAGAGAGAGAAAGGTAGAG
AGTTAGTTTAGAATTAAGCCCAGAATGCTCTTTTCCCAACACAGGTTGCATGATGACATCCTTACCTTTTCACCGCATTCACAACCCTTTATGC
TTCCTCTGTTACCACCAATCAAGTTCTCCTTCTCTCACCTCAGTACTCCCCCGTCTCCGCCCCTGCCTCATCCCTAGACCTTTCCGACTGGGAT
GGCTAACCTGTTGTAAGCCCGCAGCTTTCGGCCTGGTCTCTGCTGCTCCCAGGCGGCCCCTTTGGGTACTGCCTGAGCAAGAAGTGCTGGAGAG
GAGGACCAGTCATCAATAGGAGGATGAGATTGGGAGAGACACTCGGTGCAGGAGGCTGAGTGAGCAGGGGAGCACTAAGACCCAGGGGTAGTGG
AGGACTGCAGCAACGAGCTGGAGGAGGAGAAGTAAGCGGTGGGGGGTGGGAGCCATCTGGTACTTTGACAGCATTCAAAACAGCATCGGCCATA
ACAACAGAAATGGCCAGTCAGTCCCAAGGTATCCAGCAGCTTCTGCAAGCTGAGAAGCGGGCAGCTGAGAAGGTGGCAGATGCCAGAAAGAGTG
AGTCTCCTCTTTCCTCCCTTAGGAGTTTCGAAAGAAAATTGGGGGTGGGGGACAGCAAACATTTTGGGAAAACCCAAGGCTGGCGGGAAGACAG
CTAGGGTCTGGAGGCTGGTTAGGAGGGAAGAAATGGATGGATATTAGAATCTGGCACCTGGTTGGCTGAGAGAAGGCTGTATAACTTTCTGGAA
GGGACTGACTCCTGCTATTACATTGTGTGTGTGTGGGTCCATCCCCACTCACTGTCCTTTCTTCTGCCTCCAGGGAAGGCCCGGCGACTGAAGC
AGGCAAAGGAGGAGGCACAGATGGAGGTGGAGCAATACCGCAGAGAGCGAGAGCACGAATTCCAGAGCAAGCAGCAGGCGGTGAGTTGAGGCAG
AGTCGGGATGAGACCCCACTGCAAGTTGGTGGGTGCATCTAGTGAGGTGTGTAAGGGTGACTCAACAAGAAAATATGGTGGCAGAGGGCTGAGG
CTGAGGGGACCCTGGCAGGGACCACAACATTGGTGAAACTTTGTGATGATATGTAGGAGAGTCTGGGAGTTTTGAAGGCCACATAGAGCTTGTG
GGCGGAATGCCACAGTCTGTGTAAAGTATAACATCTATGTGGAGTATGATTAACATTTGTGGTGGAGGGTAGAGTTTTATGGTCATGGATGGTG
AGGTGGTGGGGATATTACGGTCTGTTTTAGGATGAAGTTGCATGTTAGGTCTAAGGGGAAAGGGGACTGTGTTGATCTCTTTGGTGTTGGGATA
TTTCTGTGGGATGGGGGTGGTTTCTGAGAGGGCCTTTCTTCTAGGCTTTGTTTCAGGATCTTTCCCCTCATATGCCTGGACCCTTGTCTGTTTC
TGCTTTTCCCTTTCTCTCTTCCACCCCTCTCCCTACCCCCCAGGCCATGGGCTCCCAGGGGAACCTGTCTGCTGAGGTGGAGCAGGCTACAAGG
CGCCAGGTGCAGGGCATGCAGAGCTCCCAGCAGAGAAACCGAGAGCGTGTCCTGGCCCAGCTTCTTGGCATGGTCTGCGACGTCAGGCCCCAGG
TCCACCCCAACTACCGGATTTCTGCCTAGGGCCACCGTAGGGCCTGACTCCTTCTGCCAGTTCCCTCCCTCAAAGAAATCCTCCAATCAAAATC
ACCTCCCACCATAATCCCTGTCTTCTTTCCATCCCCTAGAAATCCTGGGAGGCAGGATCCAATAATTTTCCTGTGACACTTATAAATATCCTGC
TCACATCTGAATCTCCTTGTTGTTCTTTAACCCTCACTGGGACTTTGTAAACTTCCAAGTCATTCTCACCTAAACCCTCTGTGAAATTTGTAAT
ATGGGGAAGTAGGAATGTGGAAAACATCCTGACTTCAGTGTCTGGCCGATGTGGGTCCCTCTCTTGACCCTGTCACTTGCTGGCTGTGAAACCA
GGACAAGCTACTTAACTTGGTAGCCTCGATGTCCTCCTCTGTGAAACTGGGATGATAATAATGCCTACCTTGTGAGGGTTGCTTCAATGATTAG
GAATCATTCTGTAAAGTCTAGCACAGTTCCTTGCATGTTGTAGCAGTGATTCAGTAAGTAGCAACCCTGTGATACTATTACCACCACCTGCTCA
CTGGTCAAAACCTACACAGCTGTTTCCTCACGTCCATCACTGGCTCTCTAATTCCACTTGTTCATTCTGTGACCCTAGTTATTTTCTGAAAAAT
TGGTTCTTCTCTTTTCCCAGAGACCTTCTGATCTCCAAAAAGAGGAGATGACTACATTTAGCCCCTCTCTTATAATTCCAGGTAGATAACTGCA
TTTTGTAGCCTCTCTTTGTTTTTCTTTTGCTGATCTTTGTCTTTATTAGATTTTCCTCCTTTCCTATTTCCCCAAAGACTTATCAGATGCTCAT
TGCTTTCTAAGATCTAAAATGATACTGTGTTCCCTCATATGCATGCCCTTCCTTTCTATATCCTTGACACCTTACTTTCCCATTGTAACAATAA
AAAAAGTATCAATAAAATAATTATTGGCAAATAAATTGGTGAGTTGAAGCAGCCTCCTTTTGCCTCATCATTTCTCATTTTCAGTCACTTTGTT
TTTTTTTTTTTTGAGATGGAGTTTTGCTCTTGTTGCCCAGGCTGGAATACAATGGCGTGATCTCAGCTCATTGCAACCTCTGCCTCCCAGGTTC
AAGCGATTCTCCTGCCTCAGCCTCCCAAGTTGCTGGAATTATGGGTGTGTGCCACCACGCCTGGCTATTTTTTGTATTTTTAGTAGAGATGGGG
TTTCGCCATGTTGGTCAGGCTGGTCTCAAACTCCTGACCTCAAGTGATCCACCTGCCTTGGCCTCCCAAAGTGCTGAGATTAGAGGTGTGAGCC
ACTGTGCCTGGCCTTCAGTCACTTTCTTGTTTTTTGTTTACATATTCCCTAAACAGCCCAAATGGCTATCCTTTGAAACTTCTTGGAGAAACAA
GAACAAGTAGTACTTTATTATTTCTCTAAAGTGAGAAACATGGTTCCTCATTTGGGAATCTGAGGACTATAGATCGCAACTGTAGAGAAAAGCT
GGAGTGTAGGAGCAAGTGCTCTTTGCCCCTTTACCTTGCATTTTCTTCATAGCACTTACTGCTACTGGTTTTTTGAGACAAGGTCCTGCTGTGT
TGCCCAGGCTGGAGTTCCAGCTCACGGCAGCCTTGACCCCCTGGACTCAAATGATCCTCCCACTTCAGCCTCCTGAGTAGCTGGGATTACGGGC
GAGTGCCACTATGCCTTGCTAATTTTAAAATTTTTTGTAGAGATGGGGTCTCACTTGCCCAGGCTGGTCTGAAACTCCTGGGCTCAAGCAATCC
TTCGGGCTCCGCTTCCTCAAGGGTTGGGTTACAGGCCTGAGCCACTGCACCCTGACCACTTATCGATACTTGACATTATATTTGTGTTTATGTG
TTTTCTTTCCTGTAATGTAAACACTGTGAGAACAGGGCTGTTCACCGTTGTGTCCCCAGATCCTAGGACAACATGTGGCACAAGGGAGGCAGTT
GATAAATACTTTTGAATAAATTAAATGATACTTGGGAAAATACCTTCTATGACACCATTCTTGAATTAGTTACTTCATTTGTCACTGAAGACAA
GCTTACTTCACCAAGAATTTGAACCAATAAGGTAACCTGCAGTGTATTTACTAACCAGATTCTTTGAGCAGGGAGGCAGAATACAATAGAGAAT
GAGAGATGTTTGCATCCTGGCTGTAACCTCACCAGCCGTACTGCTTGAGATATGTTGCTTTGCTTCGCTTCTGTCAATAAGATGAGAATAACGG
TACCTACTCCTTAGTATTAAATGATTAAGTATGTTAACAGGGAGAGGGCCAAACGTTTGTTGTTTTATTACACAGCAGGACATCAGGTCTTACT
TTTGTGGCTCCCCATCTCAAAGACGGGGATAGCAAATGTTTCATTCAGGAAAAAAATCCAGGTTGAACAATGGGGCTGTTGGGGCGGGGCCAAG
AACATTCTGCTCGAATTAACAGTATTAATGGGCCGGGCGCGGTGGCTCACGCCTGTAATCCCAGCACTCTGGGAGGCCGAAGTGGGTGGATCAC
CTGAGGTCATACATGGGTGAAGCCCCGTCTCTACTAAAAAAACAAAAATTTGCTGGGCGTGGTGGCGGGCGCCTGTAATCCTAGCTACTCGGGA
GGCTGAGGCAGGAGAATCGCTTGAACCCGGGAAGCAGCGGTTGCAGTGAGCCGAGATCAGGACATTGCACTCCCGCCTGGGCGACAGGGCGAGA
CTCTGTCTCAAAACAAAAACAAAAACAGTATTAATGGAATGTAGTATAACCCTCAAGCCCTACTATTAACACTTGGGGCCGAATCCAGACCCCG
TCTTCCCGCTCGGATTCAGAACACCTTCCTGACTCACTGGCCCTAGGGCATCAGCTACCTCGGACAGCATCCTTTTGGGAAAATACCGCCCACC
AGCCCCACGACTGGGAAAGAGTCGGGAAACACCCCCGAGCAATCCAGTTCCCTGAGACTTCCCTCCTCCCTCCCCTCAGCTAGGGCCTGCCGGT
TCCTAGTGCGTGCCCAGCAGTCCTCAGGTCACCTTCACTACCGGGCCAAGGACCCCGTGGGAACTCGCAGCCTTCGCCACACTCGTTCCTCGCG
CATCCACGGAGGGGTGCCTACAGAGAAGACCTGCGTGGCAAAAACCTAAACGAAGAGATGAGGGGCATGGAGAGGAGTAGGATAAGAGAATAAA
GATAACAGTGGGGGGGAGACGTTAGTTTCCTTTATATCTTTTGTTACTGGCGGTAGCAGTGAAGTTAGAAACGGTTTTAAAACAAATTTCAGAC
AGGCATTTTCCAAAGGCAAGCCTGGAGCGCACGGATCTGTATAACCGCGGAAGGCCCTGTTTCCGGTCCCTTGCGCCTGCGCTCTTGCAGCCAA
GAAGGCGGGAGGCTGGAGTAGAGGGAAGCCTGCAACCGGAAGTGAAGGCAGATTTCCCTCCTTCGTCGCTGTTGCTGCCGCCATACGCGCTCTC
CCTGTTTAGGTAAGCTTTGGCCTTCGCTACAATCCGTTTCCATCTGCGCTTCTCCGCACCCATCCCGTCACATGGGTTCCTGATACCCTTTTCA
CAGGCGATGGTCTGGTCGCTGGGGCCTAGTTGGTTCGCTATTTCCTTAGCTTGCATCCCTTTCGAGAGCAAAGAGCTCCTGGGGGAAGGAAGGG
AAGCTAAGGGGGGACCCAATCCAAGATGGTGTCCTCGGCGCCATTGTGTTCGTTTTGCTCCCTTCTTCCAATGGGTTCTTCTCATATTGGAGGC
CTCAGCATCAATGAGAGGCGGTGCTCGGCGTCCCTTGGTCTTGGTATTTGCGGAGGGCGGGGCTCTTCTCACCTTCCTTGTTCTTTCTTGAGCT
CTTTTTCGGCCCTCGGTGGGACTGGGAGGAGGAGCTGGTTTCTGGGCCCAGTTGGATTTTTCTCACCTTGACTTGCCCAACTTAATTTGGAGTG
CCTTCCAAGTGTTTACGATACGATTGGTGTCATTGTATGTTTCTCCAAAAGGAGTCTCACCTTCGTAGCGTAACAGTGATGTCAGACCACTTGG
TAAAGATCCTGTTAAAGCCTGGGCGGGGATTGCCTTTCTCTGTCACCTATTAGCTTTCTTATTGTAGGGTGGAGACATGAATTTTGTTTTTTTG
TGGCCGAGCCATTTGTCTTGCACCGCCCCTCCCCCCCATGCTAATTACACAAGGCTTGCTTAAACAGCGGAAGGGAGGATACTGAGAAGTGGGA
GGCTGAGAGCTATGGGAGGTGGACGGCGGCCATATGATGTTTTCTTTTCGAAAGGTGAGCGCTTTGCGCAGTGATGACCCTCATCTATCACCCT
TGACTGATGGCTGCTGAGTTAGGCATCCATAACGGTGGGATTATAATAGGGAAAGCGGAGTCTTCCTTTGAGGACTTTTCAGGACTCTACTTGT
CATCTCCATTTTCCACTTTACTAAGTTATTAGTCATATTTTACCTTTTATTATCTATTCTATTTCCTCACTGTTACTTTCAGATCAAGAATTTA
TAAGTTGGTCTTCCCCTTCCAACTTTTCTGGTTTCCGCTACTGTGATTGCTAATCTTGTTGGGAACCTCTGTCCTAACCACTTTCCCTGGTACT
GCTTTTTCTGTTCTGTTATATTTGCTTTTCGTTTTTATGTTTTGTATCTGTTTTTCTTTCCAGGTAAAAGTTTCCTGGTTTAGGGAAAGTGGGA
ACTGGGGATGGAAAAGAGGTCGTGATTAAGTCTTGCTTTTTTTTTCCCCCCTCCAGCTCTTCTGTTAGAAATAGTATCTTTGTTTTCCTTTGCT
GTTCCTCAATCCCCTACTCTTCACCCCTTGTTTTCACCTATTTTGCGAGAACCCATCCAGATCCCCCTTCCCTTCTTCCCCTGCCGCCCCAGTT
ATGGCAGAGAACGATGTGGACAATGAGCTCTTGGACTATGAAGATGATGAGGTGGAGACAGCAGCTGGGGGAGATGGGGCTGAGGCCCCTGCCA
AGAAGGATGTCAAGGGCTCCTATGTCTCCATCCACAGCTCTGGCTTTCGTGACTTCCTGCTCAAGCCAGAGTTGCTCCGGGCCATTGTCGACTG
TGGCTTTGAGCATCCGTCAGAAGGTAAATTTTCTCTTGGGCATGTAGTGCTCATTGGGCTCTTTAAGGGTACAATACAAAGATGTGTTTGTCGT
TGCTCAGGTGGTGGTAAGGGTTTATACTTAAGGCTAGATCAGGGCCAGGTGCAGTGGCTCACGCCTGTAATCCCAGCACTTTGGGAGGCCGAGG
CAGGAGGGTGGCCACTTGAGCTCAAAAGTGCAAGAGAAGCCTGGGCAACATAGCGAGACTCCTGTCTCTACAAAACGTTCAGAAATTAAGCAGG
TGAAGGTTGAGGCTTCAGTGAGCCGTGATTGCACCACTGTGCACCAGCCGGGGCGACAGTGAGGAAGAAAAAATCAGGGGATAAGTATCAAAAA
CAATTTTGGATAGAGGAGGCTTATACAGGCTTATTCTTTCTTTCGTGATAGCACCAAAGTGCTAATGATCCAAAAGTGACTTCCAGGTCTGCCA
TTCATTCTTGTGACTGGCTTTTCTTGTCTGCTTATTTTTAATTTTGTCACTTGACTTCTAATTTTAAATTTCCAGAAAGGTCCTGCTTGGACCT
GTAGTCTCCCTCTGTTGGGCCAGGCCAACTGTGGTCTCTGGAAACCTCTATGACTGGTTTAGAGATGACTGGCTTCTGGGTCAGGTACCAAGTC
CTTCATTTTGTCCAGGGTTGTAGTAGTTACGTGACCCGAAGTATAGCAACCTAAGCAGGAGAAGTGGTCTGTGGCAGGTATTCAAATGTCATGA
ATTGTTACAGATTAAGAAAAATAAGGACAGAGCTAGGATCATTGAAGGTGAGCGGTTGGTAGATGCAAGGGGTTTGTTACTAGGACTGGGAAGG
CCTAGATCTGGAGGAGGCTAAAGCTAGGAGGAATTAGGAGAGTCTGATTTTGAGGTGAATGTAATTGAGCAGAGAGAGGTAAAATGGGTCTGGA
AGTTGGCAAGAACCAGGTAAATACTAGACTTTGAGAATTGAGTGGTAAGAAATGGGCTTGGCATGGTGAAAAAGGTAGAGTTATCTGGAGACTG
AAGTCTAATTTATCTTCCTCCCCCCCCAACTTTTAGTCCAGCATGAGTGCATCCCTCAGGCCATTCTGGGAATGGATGTCCTGTGCCAGGCCAA
GTCGGGCATGGGAAAGACAGCAGTGTTTGTCTTGGCCACACTGCAACAGCTGGAGCCAGTTACTGGGCAGGTATATTTGGGGAGAGTGCTGGGG
AGGGGATTTTGGTTAGGACTATAAGGGAAGGGTGTTTTTGTCCTAACTACATGATGCTTGCAGAGCCATGAGCACATGACCTCTGTTACCCTTG
ACAACCTGACACCTGTGGGGGATGTTCTGTCGCAAGCGTGGGGTTCATGATTTAGATCACAGAATTGAAGTCATTTATTATCGGCCCAGGTGTG
TTTTTGTGACAGTCACTTCCCTAGAGGGGATAATGAAGAGCTACATTTACCATATGTCTCCGTCTACTTCCTGCCTAAGGTGTCTGTGCTGGTG
ATGTGTCACACTCGGGAGTTGGCTTTTCAGATCAGCAAGGAATATGAGCGCTTCTCTAAATACATGCCCAATGTCAAGGTAAGCCAAGGTAAAG
AGACCTGAGAGTGAGGGTGTGGCAAGTTGGAGGGATAAGAAACTTGTAGGCCAATAGTCTCTTTAATTTTGGAGAAGCTTTAGTTTGCTGTGGT
GTAACAGAGTGTTGAGTTCCTATGTAACAGGAGGATTCGTAATTGGGCTATGGATGATGCTTAACACAAGACCACCCTTTTCTTACTACTTTAT
ACTGACTTTGAATCATATCAGTTTAATAATTTTGGGGTATGTGGCAGAGAAAGCCGGAAACTTTAAAACAGCTCCAGTGGTGTGTGAATATTGA
GGATTCTGGCCAAGTGCACAATGGCTTACACCTGTAATCTCAGCAGTTTGGGAGGCCAAGGCGTGTGGATTATTTGAGGTCAGGAGTTTAAGAC
CAATGTGGCCAACAGGATGAAACCCTCTCTCTACTAAAAATGCAAAAATTAGCCGTGCATGGTGGCACACACCTATAGTCCCACCTGCTTGGGA
GGCTGAGGCAGGGGAATCGCTTGAACCCAGGAAGCAGAGGTTGTAGTGAGCTGGGATTGTGCCATTACACTCCAGCCTGGGTGGCAGAGTGAGA
CTCCCATCTCAAAAAAAAAGAAAAAATCTGATTGAAGTTAAGCATTTTTGGCAAGAATCCTTCATAGGTGATGCTGTATCTCCTGTTATGCCAC
AAATCTGGTCGACTTATGTTAGTTATTTTATTTTATTTTTATTTATTTGTTTTGAGATGGAGTCTCGCTGTGTCCTTCAGGCTGTGAGTGTAGT
GGCGCGATCTCAGCTCACTGCAACCTGCGCCTCCCACGTTCAAGCGATTCTCCCGCCTCAACCCCCCGAGTAGCTGGGACTACAGTGTGCCATC
ATGCCTGGCTAATTTTTGTTTTTTTTTAGTAGTGACAGGGTTTCGCCATGTTGGCCAGGCTGGTCTCGAACTCCTGACCTCAAGTGATCCACCC
ACCTCGGCCTCCCAAAGTGCTGGGATTACAGGAGTGAGCCACTGCACCTGGCCTCATTAATGATTTTAGATTTACCATAGGATTAGCGTCGTGA
CAGTCTGATTCCACAGTTCTTTTCCCCCTTGAAACCAGAAAGTAGTTTCTGGTGTTATTTGATACTGTACCAAGGCCCAGATCCCCAAACAACT
ATTCACCTAATCGTTTTAACATGAAATGATAATATTTAGCCCGAAGCAGTAATTTCATGGGGTTTGTGTGAAAAGAGTTTGAGATTCTGGGTTT
ATTTAGGAAACCTTAATGTTCCATGTGTTTTTTGTGGTACTTTACACTAATCTGGTGATTTCTTGCTGTCCTTTATTTATTTATTTTTTATTTT
TTGAGATGGAGTCTCGCTCTGTCATCCAGGCTGGAGTACAGTGGCTCAATCTCGCCTCACTGCAATCTCCACCTCCCGGGTTCAAGCGATTCTC
CTGCCTCAGCCTCCTGGCTAAATTTTGTATTTTTTGTTTTTTAGTAGTAAATTTGTAAATTTTGTATTTTAGTAGAGATGGGGTTTCACCGTGT
TGGCCAGGCTGGTCTTGAACTCCTGGCTTCAAGTGATCCACTTTCCTTGACCTCCCAAACTGCTGGGATTACAGGCGTGAGCCACTGGGCCTGG
CTTTATTTTATTTTTATTTATTTTATTTCTTTTTGAGATGGAGTATCACTCTTGTTGCCCAGGCTGGAGTACAACGGTGGGATCTTGGCTCACC
ACAACCTCTGCCTCCCAGGTTCTCGTGCCTCAGCCTCCTGAGTAGCTGGAATTATAGGCGTGTGCCACCACACCTGGCTCCTTTATTTTTTAAA
TGAAGCCTGGCCTCATAAATGAAGGAAGTTGGTTAGATTAAGTCAGTAGAACTGAATTATTGTCCTGACTGCTCCGACTAGCCATGTAACTTTA
GGCAATCACCCTCAGTGTTTGGTGGGGGGGACATAAAATTTTTAAATTAGGTCACCTCTAAAGTTAGTTTTAGTTTGGAAACATGCTAAAAATT
GGTTTAGCTCAAACAGAGTGGGAACCCTGGGGGGATTGGACTCTTTCCTTCCTCTGTTTTGAGACTCTTTGCTTCTGGCTCGGCAGGTTGCTGT
TTTTTTTGGTGGTCTGTCTATCAAGAAGGATGAAGAGGTGCTGAAGAAGAACTGCCCGCATATCGTCGTGGGGACTCCAGGCCGTATCCTAGCC
CTGGCTCGAAATAAGAGCCTCAACCTCAAACACATTAAACACTTTATTTTGGATGAATGTGATAAGATGCTTGAACAGCTCGGTGAGTGGCAGT
GCTGGGGCTTGGCTAATGCTGGGGAGTTGTTCTTTGGAGCCAAATGATGTTTATTTGAAACAGGAGCACCTCAGTGCAAGGACGACTCTTATCT
ATCACCCATGACTGATGGCTCTGGGTTCCCTGGTTGGTCTTTATTATGCTTTTAAGCACAGTAAAGGGTGTCATCTATCATCTTTCTATGATTT
TTGTTTTTAACCTTTGAGAATAGGGGACTTTGATAATTTTAGGCATAAGTCATCACCACCACCACCGTTTTCATTATAGATTCATATACTGGGA
GTCATAGGGGAGATTCTAAACTGAAAGAGAAGACAGTACCCTTCTGGCATCTCCAGCACAGCATTTACAGTCAGAATTTATAGCTGAATAAGTG
TCTAGACTCAGGTCTGCGATTAATGTAGAGAGTGTTTGTAGCAGTTTGTGTGATGTGGTATTCTAGTGTGCCAGGTGGGGTTAATGGAAGATTT
TTCTGTAAGAATTGAATCTTGGTGAATGAGAGTGGGGTTGGACATAGGCCCCATAAGTCATTACAAATGATCTTTGGCAATTCTATATGGTAAG
CTATAAAGGTGGGCTCCAGGTAGGGATGTCATATTTGCCTGACTTGATAGAAAAGTAATCCAGAGAGTCATAGATGGACTCTGATATCTGGAAT
ATAATATGTGCTTGATATTTGTAGTCTGCTGAAGGCTGGCTGGGGCTTGGGCAGGAAAGGGTTGGGAGAAGGTCCCATAAAGCATGTTTTGAAG
GCCTTGAGAGCCTCTGCACTGGGCTTTATCCCCATTTCATAGTTGGGAACTTTGGGGTTTTACCTTATTTCTTGCTTGGTTAAAACAAACAGCT
GGAATCTGATCCCACTTCTTGATTCCAAGTCCATTGCTCTTTCCATTGTGTTGTTACTATTTCCAGCAATCTTCACCTCACTGGGAAGTCTACC
TCTAATCTTTGTTTATCATACCTGCTTATTTTCTCCTACAATTTTTTTCCTTGTTCTTGTAGACATGCGTCGGGATGTCCAGGAAATTTTTCGC
ATGACCCCCCACGAGAAGCAGGTCATGATGTTCAGTGCTACCTTGAGCAAAGAGATCCGTCCACTCTGCCGCAAGTTCATGCAAGATGTAAATA
CCCTTCTACCTTCTCTCCCTCCACTCCCCGCCCGCTGCCTCCTCCCCTTCCTCGCCCTCTTCCTCAGACTCCCTTGTCATTCAAGTGCCAAGAA
GGCGGCTTGTGCCCAACTGGGAGTAATGACTCCTTGAAGAGACATACAGAAGCAGAGACAGCTAGTGTTAGGGCCTGCGCGGGTGCCAGGGAAA
CTCCGGAAGACTTGGTCGGGTTAATGTGAGAGCGGGTAGTGTTCGACTTTTTCATAAATCACAACATTTTTGAACCTCTTCTCCCTTCGGGGGA
GGGCAGGATTTTTCTGCCCTACCACCCACCCATCCATCGTCTCTTACATGCACCCTACAGCCACGCACCCTCAAGGTGGCATCGAGCATACAGC
TGGAGCCTTCTGCTCACCAAAACTCCTACTTCCCGGTGGCAGGAGAGCAAGAGAGGGACAGACAGATGGCAGGGCATGTCCAAAAGAAGAGCAT
CAGCACAAATGAATCCTCCCCTTCCCCACCTCCAGGGGTGGGGGCCTTTCGCACCTCAATCCCCGATACCCTACTCCTTCCCACCCACATCTCC
TTGCACCCATCTGGAACCTCGGTTGATGTGAGCCGGCAACAGAGAAGCACCGTGGCGCGGCGAGGGAATGCAGACGGCACCCAGCGGTGGATGG
CGGCAGCGGAGGCCGCGGGGAAACCTGACCAGGAAGCTGAGGACCAAACCAGCCTCTTTTTCCGTTCCCGGTTTTTTTCCTGAACCCAACGCGT
GCCGTGCCCCGTTTCCCCCAATATGTGTTGGGGAGGGGTGTCCTGAATGGGGTGGTAGATTTTTTTTCTTAAAAAAATTTTTTTGTTTTTTTTA
ATACTCAGAGGAGAGGGACATAGGAAAGGTAAAGTGCATGTAATCGGGTGGTTGTTAGGCTTTGGGGCTAGGTGGGGCCAATTGCATAAGCAGT
GGAGTGTGTTCTTCCCCTCCCTGCAGTGTTCCTTCCCGTGGGATGATCACTCTTTAGCTGTATTTGGGGCTAGAATGAGATTTGAAGGAGGCCA
TGGAACTTCTCTTTAGAAAGCCTGCCTTGGCTGGGCCTGGTGGCTCACCTCTAATCCCAGCACTTTGGGAGGCCAAGGTGGGAGGATTGCTTGA
GCCCAGGAATTTGAGACTAGCTGGGGCAGTGTAGTGAGACTTTGTCTCTACCAGAAAAACCGGGCGTGGTGGCGCATGCCTGTAGTCCCAGCTA
CTTGGGAAGCTGAGGCAGGAGGGTTTGCTTGAGCCCGGGACGTGGAGGTGGCAGTAAGCTGTAATTGTGCCACTGTACTCCAGCCTGGGTGATA
GAGTGAGACCCTGTATCAAAACAAAACAAAAAACAAAACCTGCCTTCTGGGATTGGGCTTCTGGTTTTTTTCCCATGACACACACATCCTTTCC
TATTTTGTCCTCTGGGTCTTCATATTAACTATCTTCCCCCAGGATAGTATAAAAAGTGTTAGGAAAGTTGGGCTTTGGAGTTGTGGTAATTTCT
GTCTTTGTTACTTTCCTCCCCTTCAGGGGGTTTTTTAATTTTAAAGATGAATGCAGTGAGGTATAATGGTGTGTGCCTGTAGTCTCAGCTATTC
AGGAGACTGAAGCAGGAGGATCACTTGAGCCCAGGAATTTGAGGCTATAGTGTGCTATGATTGTGCCAGTGAATAGCCACTGCACTCCAGCCTG
GGCAACATGGTGAGATCCTGTCCCTTAAAAGCGTATCTGCTGCTCTGAATTTGGTATTTTAACACCACTTACTGATACCTTTCCTGTAAACCTG
TAGATGGTTTAATTCTTAGTCAAGAGACCAGTCTCATCTAAAACTATCCTGTTGTGGTCTGACGGCAAGTAACTCATCTTGAGTAATTTTTGTT
TCTCCTTAAGTGGCATTTTGACTGTCCATTGCAGCATTCTGATCTTAAAAGACATCCACTTTGCTAATGCACACGAGATTCTCTTAGTTGAAGT
AGGAGAATCAAATGGAGCAGTTGTCCTCCCCCCACCCCATGTTCTTAGAAGCACCTCTGATGGAGTTATTCTGACCTTGAGTCACTGCCTCCCA
TCATTTCCCAGATGTTTGGTCCTTGCTCTCCCTTTGAGAATCATCTCCCATTTTCTTTCCTCTCCCACCTCTATTTGAGGTAATGGCATCTGTG
CCATTGGGTGGTTTCACTGCTCCTTGACTTCATTTGCAGTTTCTTTCCCATGATAGTTTTTAGTTGGGCAGTCTTAAAACTCATCTGATAGGAA
GGAAATTAGATGTAATGTGAGAGAGACCACAGTAAAATGTGGGTATTTTTGGGAGTGGGGTGGGGTTTTCAATCTTCTCTTTCCTCCCCATCCC
CCCATGGGGTGTATTGGAGATCAACTTCCTCCACCCCCCCAGGTTTAACCCCCCCACTCTGCCCTCCTCCCGTTCCCCACCCCCTTCCTC
HUMAN SEQUENCE - mRNA
CTAAAGGCTGCCGCCATACGCGCTCTCCCTGTTTAGCTCTTCTGTTAGAAATAGTATCTTTGTTTTCCTTTGCTGTTCCTCAATCCCCTACTCT
TCACCCCTTGTTTTCACCTATTTTGCGAGAACCCATCCAGATCCCCCTTCCCTTCTTCCCCTGCCGGCCCAGTTATGGCAGAGAACGATGTGGA
CAATGAGCTCTTGGACTATGAAGATGATGAGGTGGAGACAGCAGCTGGGGGAGATGGGGCTGAGGCCCCTGCCAAGAAGGATGTCAAGGGCTCC
TATGTCTCCATCCACAGCTCTGGCTTTCGTGACTTCCTGCTCAAGCCAGAGTTGCTCCGGGCCATTGTCGACTGTGGCTTTGAGCATCCGTCAG
AAGTCCAGCATGAGTGCATCCCTCAGGCCATTCTGGGAATGGATGTCCTGTGCCAGGCCAAGTCGGGCATGGGAAAGACAGCAGTGTTTGTCTT
GGCCACACTGCAACAGCTGGAGCCAGTTACTGGGCAGGTGTCTGTACTGGTGATGTGTCACACTCGGGAGTTGGCTTTTCAGATCAGCAAGGAA
TATGAGCGCTTCTCTAAATACATGCCCAATGTCAAGGTTGCTGTTTTTTTTGGTGGTCTGTCTATCAAGAAGGATGAAGAGGTGCTGAAGAAGA
ACTGCCCGCATATCGTCGTGGGGACTCCAGGCCGTATCCTAGCCCTGGCTCGAAATAAGAGCCTCAACCTCAAACACATTAAACACTTTATTTT
GGATGAATGTGATAAGATGCTTGAACAGCTCGACATGCGTCGGGATGTCCAGGAAATTTTTCGCATGACCCCCCACGAGAAGCAGGTCATGATG
TTCAGTGCTACCTTGAGCAAAGAGATCCGTCCAGTCTGCCGCAAGTTCATGCAAGATCCAATGGAGATCTTCGTGGATGATGAGACGAAGTTGA
CGCTGCATGGGTTGCAGCAGTACTACGTGAAACTGAAGGACAACGAGAAGAACCGGAAGCTCTTTGACCTTCTGGATGTCCTTGAGTTCAACCA
GGTGGTGATCTTTGTGAAGTCTGTGCAGCGGTGCATTGCCTTGGCCCAGCTACTAGTGGAGCAGAACTTCCCAGCCATTGCCATCCACCGTGGG
ATGCCCCAGGAGGAGAGGCTTTCTCGGTATCAGCAGTTTAAAGATTTTCAACGACGAATTCTTGTGGCTACCAACCTATTTGGCCGAGGCATGG
ACATCGAGCGGGTGAACATTGCTTTTAATTATGACATGCCTGAGGATTCTGACACCTACCTGCATCGGGTGGCCAGAGCAGGCCGGTTTGGCAC
CAAGGGCTTGGCTATCACATTTGTGTCCGATGAGAATGATGCCAAGATCCTCAATGATGTGCAGGATCGCTTTGAGGTCAATATTAGTGAGCTG
CCTGATGAGATAGACATCTCCTCCTACATTGAACAGACACGGTAGAAGACTCGCCCATTTTGGAATGTGACCGTCTGTCCTTCAGGAGAGGACA
CCAGGGTGGGGTGAAGGAGACACTACTGCCCCCACCCCTGACAGCCCCCACCCCATGGCTTCCATCTTTTCCATCACCACCACTCCTGAACCCC
CATTTCTGATTTGTCAGAATTTTTTTTTTAACAAAACTAAAAATGAAAAAAAAAAAAAAA
HUMAN SEQUENCE - CODING
ATGGCAGAGAACGATGTGGACAATGAGCTCTTGGACTATGAAGATGATGAGGTGGAGACAGCAGCTGGGGGAGATGGGGCTGAGGCCCCTGCCA
AGAAGGATGTCAAGGGCTCCTATGTCTCCATCCACAGCTCTGGCTTTCGTGACTTCCTGCTCAAGCCAGAGTTGCTCCGGGCCATTGTCGACTG
TGGCTTTGAGCATCCGTCAGAAGTCCAGCATGAGTGCATCCCTCAGGCCATTCTGGGAATGGATGTCCTGTGCCAGGCCAAGTCGGGCATGGGA
AAGACAGCAGTGTTTGTCTTGGCCACACTGCAACAGCTGGAGCCAGTTACTGGGCAGGTGTCTGTACTGGTGATGTGTCACACTCGGGAGTTGG
CTTTTCAGATCAGCAAGGAATATGAGCGCTTCTCTAAATACATGCCCAATGTCAAGGTTGCTGTTTTTTTTGGTGGTCTGTCTATCAAGAAGGA
TGAAGAGGTGCTGAAGAAGAACTGCCCGCATATCGTCGTGGGGACTCCAGGCCGTATCCTAGCCCTGGCTCGAAATAAGAGCCTCAACCTCAAA
CACATTAAACACTTTATTTTGGATGAATGTGATAAGATGCTTGAACAGCTCGACATGCCTCGGGATGTCCAGGAAATTTTTCGCATGACCCCCC
ACGAGAAGCAGGTCATGATGTTCAGTGCTACCTTGAGCAAAGAGATCCGTCCAGTCTGCCGCAAGTTCATGCAAGATCCAATGGAGATCTTCGT
GGATGATGAGACGAAGTTGACGCTGCATGGGTTGCAGCAGTACTACGTGAAACTGAAGGACAACGAGAAGAACCGGAACCTCTTTGACCTTCTG
GATGTCCTTGAGTTCAACCAGGTGGTGATCTTTGTGAAGTCTGTGCAGCGGTGCATTGCCTTGGCCCAGCTACTAGTGGAGCACAACTTCCCAG
CCATTGCCATCCACCGTGGGATGCCCCAGGAGGAGAGGCTTTCTCGGTATCAGCAGTTTAAAGATTTTCAACGACGAATTCTTGTGGCTACCAA
CCTATTTGGCCGAGGCATGGACATCGAGCGGGTGAACATTGCTTTTAATTATGACATGCCTGAGGATTCTGACACCTACCTGCATCGGGTGGCC
AGAGCAGGCCGGTTTGGCACCAAGGGCTTGGCTATCACATTTGTGTCCGATGAGAATGATGCCAAGATCCTCAATGATGTGCAGGATCGCTTTG
AGGTCAATATTAGTGAGCTGCCTGATGAGATAGACATCTCCTCCTACATTGAACAGACACGGTAG

TABLE 3
MOUSE NOMENCLATURE
ICSGNM Iqgap1
Celera mCG16312
HUMAN NOMENCLATURE
HGNC IQGAP1
Celera hCG27443
MOUSE SEQUENCE—GENOMIC
TCATGGATGGCTGTGAGCCACCATGTGGGTGCTAGGAACCAAACCTGGAGCCTCTGCAAAGGCGACTTGTGCTTTTAACCACTCAGCCGTCTCC
GGGCCCTGAGCCGAGACAGCCTGGTAGAAAGCCTCAGCCCAGCATGGAGCTGCCTCCCAAATTCTGGCATTCAAGGTGTGCACCACCACTGCCG
GCTTGTCTGCTTTCAAAGAGCTTTAAGTGGGCACGCACATGTGTGGTGGTCCCAGGACAACACTGTAGAGTTGGCTTTCCCTTTCACCTTTATG
TCGATTTCAGGATTAAACTCAGATTGTCTGGCTCGAGGAGGGGGTCTTACCCACTACCCTACTCCAGTAGACCTGTTGTTTTGTTTTGTTTTGT
TTTGTTTTTTTCTGGCAAAGGGTCTGTGCTGGTTAATTTATTGTCAACTTGACACACAAACTGGAGACACCTGGGAAGAGGGAACCTCAATTGA
GGAACAGCCTCCATCAGATTGACCTATTGGCAGGTCCGGGGGATATTTTCTTGATTAATGATTAATGTGGGAGGGCTCAACTACTGTGGGTAGT
GACACCCCTGGGCAGGTGGTCATGGACTGTATAAAAAATAACAAATTAAAAAACAAACCAAAAACAGCAATAACAAGCAAGCTGAGCAAGCTAT
GGAGAGCGATCACTTTAAAGCAACATTCCCTTTCTGCTTTACAAACCAGTTCTGCCCACAGGCTCCTGCTGTGCGGTCCAGCTTGGCTTCTCTT
GATGATGAGATGATGAATTCCAATCATAAGATGAAATGAACCCTTTCCTTTTCAAGGTGGTTTTGGTCAATATGTTTTCCTTTTTCTGTTTTTG
TTGTTACTTTTCTTTTTCCAAGATTTAAAAAAAATTTTTTTAAATAGTGTGTGTATGTGTGGTGTGTGCCTGAGAGGCCCATGACATGCATTTG
TGAATGTCAGTGCAGTTGCCCATGGAGGTTAGAGGAGCAGGACCTCCCTAGAGCTGGGGTTAGGGGCAGTTGTGAGTTGGCCTATGTGAGAGCT
GGGAATGGAACTTGGGTCCTCTGCAGGAGCAGCCTGCGGAGTTGTTTCTCCCTCTGCTTGGTCAGTGTTTTTCACAGCAACATGAAAGACTAGG
AGAGGATCTTATGTAACACAGGCTGGTTTTGAACTTGCTATGTAGTTGAGGCTAGCCTTGAACTTCTGATTCTCCGACCTCCACCTCTCAGTCA
CTGGGATTCTGTGTGTGTGCTGCCATGCCCATCAGTACCTAGTTTAGCCAATATCAGTCCCCAAGAACAAGAAGTATCAAAGGAGAAAAGCACT
CTAGCGCCTCTCATCCAGAGCATCCCCCATCATCCAGCAGCCCAAGGTACATAACAAAGAGAGGAGATGACACTCTCCCAGGGGCACGGCATGC
TTCCAGAGTCCACTTTGACTTTAGGAAACATTCACAGTGTTTCCTAAATGACTTCTGCTCCTGGAGAAGACTGGAGTGAGGACAGCCAGGCAGT
CAGAAGGGCCACATTCACATGGTCACACCCACATGTACCCTCCTGATTTCCTGGCTTTGTTAACTTCCCTTCCATAACTCTCTGTCTCTCTCTG
TTTCTGTCTCTCTCTCTCTCAGTCTCTTGTCTTCCCCCAGCAGTTCTCAACCTGTGGGTCTCAACCTCTTTCAGGGTTGAATATCAAATATCTG
GCATTTCAGATATTTACATCACAACTCATAACAGTAGCAAAATTACAGTTATGAAGTAGCAATAAAAATAATTTTATGGTTGGGGGTCACCACA
GCATGTGGATTAAAGGGTTGCAGCTTTAGGAAGGTTGAGAACCACTGCCTGAAAACCTTAATCAAGCAACATTTCTAGAAAACCTCTTTATATG
ACCTAGTAAAAAGTTTTTACTGAGGTGTGGTAGTATATACACCTGTAATCCCAGTACTCAGGAAGTAGAGGCAGGAGGATTGTCACAGTTTGAG
ACCAATTTGGTCGACAAAGAGAGTCCTAGGCCAGCCAAGGCTACATAGTTAAACCCTGTTACAAACAAATAAACCTGAAAAACAAAATGAAGCA
GAGATGTGAAAGGACTGAGCGAGGAAAGGTGCTTGTTACCAAGTCTAGGATGTGGTAGAAGGAAAGAAGTGATTGATCCCTGAAAGTTGTCCTC
TGACCTCCTCATGTATGTATAAGTACACACACACACACACACACACAAACACACACACACCACCACCACCACCCCCACCACCCAACCCCACACT
ATACAGCAAATAGTAACAGCAAAGTTCCTGGCATGGTGGCACACACCTTTAATCCCAGCACTCAGGAGGTAGAAGCAGGCAGAGCTCTCTGAGT
TTGAGGGAAGCCTGGTCTACAAAGCGAGTTCCAGGACAGCCAGGACTGGTCCACAGTCCTGTCTCAAAAACTCTGTCTCAAAAAACCAAACTAA
CCAACCAACCAACCAACCAACCAAACCAAACCAAAAACAAATAAACAAACAAAAAGACCAAAACCAAAACCAGTAAAGCAAAGCAAAGCAAAAC
AAAACAAAACAAAACAGCAAAAGAGTACCATCACTAAAACTGCAACACAACACAACACAGCACAACAACAGCTTTAGTCCCAGATCTCCGTCAC
AAAACTGGAAAGTTCCTTGAGAGTACTTTGACCATCTGTGGCCACTACATCTTCTGTCTGTTTGAGACAGGGGCTCACTATCGGACCCTGGTGG
TCTGGAACTCACTATATAGACAACTGCCTCAAATTTGTGACAATCCTGGGAATAAGGCCTGCACCACCACACCTGGCTTGTGACCATCATATTC
TTTGCTGAGGCTTCACAGATCTGTCAACAAGACTTGTAAGGTGGCTGCTGCCTGGAAAGACACAGTGTATCCGTGGAGAACAGAGCTCATTCTT
GGAGGGTTTGGGCCAAGTGCAGTTTAAATTCTGGCAGTGTTCATATTTAATCTTCAGAAAAGGAGTTAATCTGTCAGATCTCGGTTCCTTGAAT
TGCAGAATTCTACACACACTCACACACACATGTGAGAGCACATAGACATACAGACACACACCACATACACAGAGATATACACTAAATAAAAATT
TCTTTTTAAAAAATGAGATGGTACATATCAAATACTCATAAGAGGACCTAAATGGCTGGGCATATAGTGCATGTTTGATGTGTATGAGACCCTG
GATTTGGTGGCCAGAACTACCAGAACAATGTCGTGTTACAGAAAAAACAAACTGCATCAGAAGGCCAGTGCTACGAGTTAGCTATTGATACTTA
TAATTCATTGACATCCCTGGAGACCTGCTGGGGCAATCTTGTCTCAGAACCTGCTCCGCACTGAACTGTGCTATCTCTGGCTCTAATTACTTCC
CTTCTCCCACACTTTAGAGAGCCGGTAGAACAGAAGTCTATAACAAGGCAGGTGCATTGACTCGTTTTATGTGAGTCTACCTGCACGGTTCGAG
GGTGGGGAGCCTGAAAACACCACAGCTGCTCAGTCTGCACAGCTGGATGCCTCAGTAGTCCTAAGTGGTGCTGACTTCCTGGGTGACTCCTGGA
CAGCCCTGGTCTTCAGTCTGCATTGGGAGGCTGAGGCAGCTGCATGCTAATGGCAGGGACAGTGGCTTTGACCAGACAAAGGAGATCAAACAAT
TGACACCTTTTGTGGAACTTCCCTGGAGGTGGACCATCCTGGGAGGTCCTGCCCACCCTGGGTGGGCTTTCTTCCTCAGTTGTCCTTCATGGAA
ATATCCTATAGACCTTCTCAAAGAATTTTTTAGTTGAGTCCGGAGCCAATCAAATTGGCAGTTAAGATTAACCATCACAGAGCACCTTTGATCC
CAGCACTCTAGAGGCAGAGGCAGGTGGACCTCTGAGTTTGAGGCCAGCCTAGTCTAGAGAGAGAGAGAGAGAGTTCTAGGGCTTCAGAAAGAAA
GGCTGTTTCAAAACTACTCCTTCCCCCAATATTAACCAACACAATTTGTCTCTCCCTCTTTCTGATGTTTTTAGTTGAGATGTCAACAAAGACC
AATGGCGGAGCTCGAAGGAAGCCTTGGTGGTGGTGTCCTGTCACTTGGTAGGTGCAGCTCTCTTCCTTCCACCTCTAGAATTATGTTATCCAGG
GTGTTCAGTGAACAGGACAAAGAAATGAACTCTACTTGTGTAACCAGATTATTATGGGCTACCTCTAATCTGTCAGAAAGAAGCAAGGGGCTCA
GCTAGCTCATAGTTTTTATTTATACAAGCTTTTCTCATTAGTTTATTTGACAGTGCTATTTAGGAGCCACCTCAGACTCCTCTTCAAATATCCA
AAATGTCCCTGGAAACTTATATTAGTTTTCTATCAAAATCCAAGTACGTGGCTGCTTCCTCTCTTCCTCCTTCTCCTCCTCCTCTTCTTCCTCC
TCTTTCTCTACCACCATCTTCTTCTTCCCATTCCCATTCCCATTCCCATTCCCATTCCCATTCCCACTCCTATTCCTATTCTTCTTTGGAGACA
GAGTCTTGCTATGTCGCCCAGATTGGTCTCAAACTCACAGCAATCCTCCTGCCTTTATCTCCTGAGCACTAGGATTATAGGCATGAACCACCAT
GCCCAGTTTATCAAGTTCCTGGGGACTAAACACGGGGTTCTAGCATGCTCTCTATCAACTGAGCCACATTCCATCTCTGATGTCACCCATCTTT
AAGCAATGATAGGTTTCTTCTCTGTGCGCTTTTTTCATGTGGTGCTTAGCTATTTGGCTTCCGTCTGTTCGCTTTGTGCTGATTCTCCACCTCC
CTTGCTCTCAGAAGCACATACATGTGAGCAGCTTTCCATTTAATTTTGGGGATGTATGATTTCTGAAATGTTATTGGGAACCCAGTAAGGAAGT
GACTGAAGGAAGGCAGCAACATCCCATTTGGGGCTGGGACGTTAAGTGGGAATACCCCAGGCAGATGAGAAGCTAAGGTTATTCCAGGCTATCT
TAGTCAGGGTTTCTATTCCTGCACAAACATCATGACCAAGAAACAAGTTGGGGAGGAAAGGGTTTATTCGGCTTACACTTTCCACATTGCTGTT
CATCACCAAGGAAGTCAGGATTGGAACTCAAGCAGGTCAGAAAGCAGGAGCTGATGCAGAGGCCATGGAGGAATGTTCATTACTGGCTTGCTTC
CCCTGGCTTGCTCAGCCTGCTCTCTTATAGAACCAAGACTACCAGCCCAGAGATGGTCCCACCCACAAGGCCTTTCCCCCTTGATCACTAATTG
AGAAAATGCCTTACAGTTAGATCTCATGGAGGCATTTCCTCAACTGAAGCTCCTTTCTCTGTGATAACTCCAGCTGTGTCAAGTTGACACAAAA
CTAGCCAGTACACAGGCCAAGGGAAACCTCTGTCATGGGTGCTTATCTCTAGCCATTGTGTTAATATTGCCCATTCCCATCAGCTTATAGATCT
CTTGAGGTCATGAACCCATGTGAGTTCCTAAAATGACCAGATAGCTCATTATTTTGCCATTTGATGCCTACTTATGGATTGATTTGGTATGTAA
TGAACTTTCTTTGTGTTTGTGTTCACTGTGATATTGTATTTCTAGTATTTCACTGTAAAGAACATAGAGACGTAAACAAACAAGGTCCACAAGT
CCAGTAACCTCACTACCTAGAGAGACTACTGTTAATATATTGTCCTGGGTGCTTCTCTTTTCTTTTTTGAGACAGGATCACATCATGTAGCTCT
GGTTTGTCTAGAACTCTCTATGTACACCAGGCTACCCTGGACCTCATAGCTATCTGCCTGTCTCTGCCTCTAGGCATGAAAGACAAGCACACCA
TCATCCCCAAATCTCTCCCTTCTGACCAAGAGAGGTTCTTGAACCTATTATTCTTCTGTCTTAGCCTCCTGAGATCAGGGCTATACAGATGCAT
GTCACTATTCCTGACTGTTTTCTTTCTTTCTTTCTTTCTTTCTTTCTTTCTTTCTTTCTTTCTTTCTTTCTTTCTTTCTTTCTTCCTTCCTTTC
TTTCTTTCTTTCTTTCTTTCTTTCTTTCTTTCTTTCTTTCTTTCTTTCTCTTTCTCTCTCTCTCTTTCTCTCCCCCTCTCTCTCTCTTTCTTTC
TCTCTCTTTCTCTCCCCCCCTCTCTCTTTCTTCTTCTTCTTCTTCTTCTTCTTCTTCTTCTTACTTCTTCCTTCTTCTTCTTCTTCTTCTTCTT
CTTCTTCTTTTTCTTCTCTCCTCCTCCTCCTCCTCCTCCTCCTCCTCCTCCTCCTCCTTCTTTCTTCTTCTTAGAAAGAGTCCGAGGTATCCCA
GGCTGGCTTCAAACTCAGTATGTATCAAAGGCTAGCCTTGAGCTCCTGATCCTCCTGACTCTTCCTCCCAAGGACTGGGGTTATAGGTGTGTAT
TACTGCCGTTGGATGTCTTTATATTTTAAAGCAAAAATGGTATCATAATTTGTGAGTTCAGAATTTATCTTCATTTAAACATCACTTCAGAAAT
CCTGATGTCTCTTGCTCACCCAGCTTCCCACTAAAGAACTGCAGATTCTCTTCTGCGTATGGTGGCATGTCTCAGACCACCCACACAAGTTGAA
AACACAGTGAGCTGAAAAATGTGCAGATGCCACGGCTTAATCTCTCTGCCCAGAGTAGGGATGCTATCCTTCAGCACGACATCTGCTCACTATC
TCTAGCCCAAGGAAGAAAATTACAACTTAAAATTTGAAGTCTGGTTCCTATTGATCATGTATCTTTTACAGCATCAAAAAAATTGAAAAGAATC
TCTTCATTTGTTACTTTGTGTTGCTACAAGTTCTTACTATGTGACTCAAGCTGGCTTCAAATTTGCTATTCTCCTGTCTTAGCCCTCCCACGTG
CTATGATTAATGTATGTGTACCTCCATGCTGAGTGGTGATTAGAGCCATTCTAAGACCAGGGGCTATCAACTCTAATATCCAGTTCCGTATTCT
CTATTCTTAATTGCCATTTATAGTTCAAGGGCAGCCCACCTATGGACTCATATACTAGTAAGGTAAAAAGATGAGCTGAAATAGTTACATGTCA
CTCCTGATCCTCCTGCCCTCTCTCTCCCAAGGAACCATTACCACAGACTTTGTATCTGTCTCTCTACAAATCCCAGGCTGGAAATCACAATGGA
AGGCAGGCCAGGCTGGAGTGGCCCTAAAGAGCTTCAACCCTGTGCTCAGTGACCTTAGACCAGAATTGCTCTAATTTTCATAGCAGAGATGGCA
GGACTAATTCTCACATTCAAGATGGCTCCTATTAACCTAGAATTGTATAGCATAGGACCTGGACTGCCCTCCTTTGAACCTCACGAATTATACA
CCTAGAGCTGTACACACACACAAAAAACCCTCATCCTAACATAGGAAGACACCTGTCTCTTTGTCCCATGTGACCCCTTTACTATTAACCTACA
GGTAGTTCTCGACTTGCCATTAGATAATTGGATAAATTAAAAAGAAAATCAAATATAGTTTTGTAGACATTCTTTTAAATGCTTGAAGATACAG
AAGTAACCAAGTTACTGAGTGTAACCCAAATTGTTTGGTTTTCAAAAAGCTACTGGTATTGTCTGGATAGATGCCTCAGAGACGGAGGGAAAGC
AGTGGCTGCTCTTCCACAGGACTGGGGTTTGATTCCCAGTGCCCAGATGGGTTACAATTGACTGTAACTCCAGTCCCAGGGGATTTAATCCCTT
TCTCTGGCATCTGAGGGCACCAGGAATGCACATGGAACAGAGACACATGAATAGACAAAGCACCATACACTGGGGCTGGAGTTAAGAGCTGATA
AGGGGACCTGGGTTTGATTCCCAGTACCCACATGGTAGCTGACAACCAACTGTAACTCCAGTTTGAGGGGATCTCCTACCTTCCACTGACCTCA
GATGCCAGGTAACCACAAGGCACACATACGTACATAGATAGAACTAGGGCCTCATGCATGCAAGGCAAGTAAGTGTTTTGCCTCTGAGTTTTAT
CCCCTGCCCTCTTGTATGCTTTCTCTTTCCTGTTTCTTTATAGACAGAATCTTGTACACACCTGCCTACCCCAGGTTTCGAACTTGCAGTGAGG
ACTTCTGCCTCTGCCCCAAGAGTACTAAGATGACTGGTGTAGTGCAGCTGCTTATAGATTGTAAGTTATCTTCTTCCTTTAGGAGCTCAGGGCT
ATGGGTGTAGCTTAGTGGCAGAGGGCTTGACTAGCGCATGTCAGGGCATGGGCTGTATCCTAAGCACTATATAAAGTTCTTAACTTTTCATTTA
TTTATCTTGACACACAGACACACTCTCTACACATTTCTGGCTGTCCTGGAACTCACTATGTAGACCAGGCTGGCCTTGAACTCACCGAGATCTG
CCTGCTATTGTCTCCTGAGTGCTGGGATTAAAGTCGTGCCTCAACATGCCCAACTAAGAAGAAAGTTTGTAAGGAAAGAGCTAGTAAATTGAAA
ATGGCTGGAACAAAGAGCCTGGAATTGGGTTGTAAAGGTGCCCACCTGGCATGGGAAAGTACACAAACTGCACTGTGAAGGGAGTAACAGGAAA
AATTACATCTATGACAAGAACTCGGAAGCTGGGTGTGGTGGCACATGCTTTTAGTTCTAGCAATCAGGAGGAAGAGACAGGTGGATCTTCAAGG
GTAGCCTTGCCTATTTAGTGGGCTCCAGGACAGCCAGAACTACATAGTGAGACTTGTCCCCCTTCCCAAAGAACTCCGTACTTTGGAAAGAGCA
TGCTGGCAATCATGATGATCTGGTTGGCACAAGTGAGAATAGAGATTATGATGATCAAAAAAGTGTTATCACAGGTGGGGACAGAAACAAAACT
AGATGCTTCAGAAATAGAGGTGAGTCGAGGAACCTGGGGTGTGTGTGTGACTGGTGATGGAACACTTCCAGAGAGTGGGGCAGGAACAGGATCA
ATCGTCATCCCCCAGTTTAAGAGATTGAGCAGTTCTATTCTTTAGCACTGTTGGGGAGAACAGAGGTCAGATTGACAGAATGTAGCAGAGAGTA
GGAGTGGGAGTCTTTCCTGACAGTGGGTCCTACTTGCTTCCAATAATCACACTCTGGGCCACTTGCAACGGCTCCTTGAAGGAGCCGCGGTGCA
GAGGGGGTTAACTCAGTTGTCAGGAGAAGGCAGGCGCCTAAAAACGTTTGGATTTCAAGCTGTAGACATTGGCGGCCTTTTAGTCAGAGCCGCA
GATAGGGCGACAGCGGAGGAGTGTCGGGACAACGGACCAAGCCGGCCTAGGGACTCGCTACAGCACCCCGCCTCCTCGCTTCCGCAAGTGGAGA
GCGAAGAGGGCAGGGGCGGGTCCTGTAATACTGACCACGCCCCTTCCGCTCGCTCCGGGGAGCGCAGCTGGGCGTGGACTCAAGGGCGGGGTCT
GCGGGGCGGGACCTGCAGGGTGGGGCCGATGGCGCGGGGCGGGGCTCTGAGGACAGCGACAGCCCGCGCACTGGGCAGGAGTTACTGCTGCTAC
GGTCGCCCGCGTCTTCAAGGTCTCTGCGCTTCCTCACCGGAGACCTGGACTCGGCCGCCATGTCCGCCGCGGAGGAGGTTGATGGCCTGGGTGT
GGTCCGGCCGCACTATGGCTGTGAGTGCCGAGGGCTTGGGGGTGCGCGGGCGAGGGACCAAGCTCGGGTTTTCCCAGGGGCTCAGCCGGGCGGG
TGCGCCGGGTGGCTTGGGACCGCACGGACCCGGAAGAGCAGCTGGCTGTATGGCTGGTAGCAAGGTTGGCGCCCGAGCCTTCTCGAGCTCCGGA
GGCGGCGGGTAGAAACCTGGAGGCCTCTGCCTTCGCTAGGCGCTGCGGCAGGGGAGCCCCACCCAGCTCCTCCACCGGTTAGTTCTACTGAGAT
TGGGGAAGACGGCCTAGGGTGAGGTGGCAGGAGAACTGACTGTGCAGGGCGTGTCCTGGGCACTGGACCCAGGGGTTCTGCTCGGGCGTGGTGG
GAAGGTGCTGCCCGGATTGCTGCCTGCGAGGGTGACCCAGGGCGCAGACCCGCGCAGCCTCAGGCTAGCTGCGAGGTGCAGTTTTAGGCAAGCA
GCTTATGCTTTCTCCTGCTGGGTTCTTTCTTTAGCATTTCAAAAGTAAAATTAAAACATAAACACACCCAAACCGGCTCCCCCCACTTCCTCAT
TTTCTGTGACTATCCCTGCAACCTTAGTAAACTAACCAGCTGTACGTGGGATAGTAGTTACAAGAGGATCCCTTTGAGTCCCAGGTTTAGATTT
TATGTTTTGGGAAAAGTACTTTCTTAAAGGATACTTTAACACATCCAGTTATGCATTGGATAGTGTTATAATGTGTGCTACGTTTGTGAAATTA
GGGATGAAAAAGATCAAACAGTGCCTTCAAAACCTGTCCGAGAAAGAGACAGGTGTACAGAGATTGATAATTAGATGAGGGGTTGGGAATTTAG
CCCAGTGGTAGAGTGCTTGCTTAGCAAGTGCAAGTCCTCAGCTCCAAGAAAAAAACCAATAAGGTGCAATAAAGATAAATGATGCATGAACTAA
CTCTAACTGGGTATAAAGAGTAATGGGGGTGAAGAGAAGGGACTTAACAAGGCCATGGGAAGGTAGGTGCTGACTTTTAAACAGAGGCTTGGAG
GATGCAGTGTGGGGCAGGGTGAGCAAGACATGACTTTGAGAGGGCAGACACAAACTAAATATGGTATTGGAGAACATAGGGTTACTGTACGTGG
AAAACCGAGGGCATCGAAGTGCAGAGGTGGAATTGAAAGGGAGAGGAGAAAAGTAAAAAGGATTTCCCGAAGTAGCCCAGGCTAGAGTGAGAAT
AGCTCTGATTGCCTTCCCAAGGAGTTATCCCTTGGGAAGACAGTGGAGAAGGCAGATGGCTACAAGGTAAAGTCCACAGGCCCTGGAGTCAGGT
GGGCTGCAAAGGCCCTTGATTAGCCCTCCGACTGAGTTCTTCCAGCCTCAGTATCCTCAGCTGTCAAATAGTGGTGATCACAGGATGGGTCTGG
GCTAGAGTAGGAGCATGTGTGTAAACTGTGTAGTCCAGGAGCTTCGGTTATGAAGAGAGGGGAGAGTGTGACCAACAAGGATAGACTATAGTTG
ATGCCTCTTGAAGCTACAAGAAAATGTAGTACATATATATTTAAAACACTTAAAATTTTGGTTCCTTGATAATTTCTTTTTTAAAGATTTATTT
ATTTATTTTATGTATATGAGTACACTGTAGCTGTACAGATGGTTGTGAGCCTTCATGTGGTTGTTGGAAATTGATTTTTTTAGGACCTCTGCTC
GCTCTGATCAACCCCTCACTCAGGCCCAAAGATTTATTTATTATTATAAATAAGTACACTGTAGCTGTCTTCAAACGGAAAATCATTTTGTAGT
GCCCCTGGCTACCCATATATCCACTAGTTAGCCTGCCTTTCTAGGTGACTAGCCAGCAATTTGATGGCGGTGGCCTTCATTTATTGCTGAGGAA
AGTTACAGAGCTTGGTGTTTACACTCTTTCCTTTCTGTTTTAGCCGTCCTGGATAATGAGAGGCTCACTGCAGAGGAGATGGATGAGCGGAGAC
GGCAGAACGTGGCTTATGAATACCTTTGTCATCTGGAAGAAGCAAAGAGGTATGGAGGGACTAGTTGGAGAGTGGCCTGGGAGAAGGAGCTGAT
GTGCTGTGCCTTTAAAAGAGTTGTTTTCATTTCTGTGTATGGTTTCTGGTATCCATGTATGGCTGCCAGTGGAGGTTAGAAGGCCTTAGATCTC
CCCTGGGGCTGGAGTTACAGTCACCTGATGTGGGTGGTGGCAGCCCAGCTCTGGTCCTCTGAAAGAGCAGCAAGTGTTCCTAACTGCCGAGCTG
TCTACCCAGCCTCCACAGGTGGCTCTCTGTGTGCATTATGTTTTAGGTAAGAGTGTTGGCGGCTGGCAGTTGAAAGGGTAAGTAGGTAGGATAA
GTGTAGAGTGTTTATTCATGGGAACAAGTGGGCCGTGGTGGGTTATGATGAAACACTCTGTCCAACACTTGTGCCTGCTGAAAGATCTCCCACA
GTTGTGTTTTTCAGCCTTTCTGTTTGTAGTGGTTGTTTTGGGTACTGCCAGCTGTCAGAAATAACGTTTTAAGTTAGGGTTTGATGTTCTTGAT
CTTCTCAGTGGTTCCTAATTCTTCAGGGTTTTGGTTTGTGTATTGAGCCCCATCTCCTGGTGACTCATGGAGTGGGTATTAGGTTTAGGGGGCT
GGTAAGGGTGGAGCTGAAAGGGGCTGAGGACATACCTGAGTGGTAAAGTACTTGTTGAGTGCTCAAGGTCCTGGGAGACACCCAGTGCCAGAAG
AAAACTGCAGAACCTTCTTACGAGGGTCATGCCGTAGCTATAGATCTGTCCACCAGCAGCATGGTGAGGCCTTTCCGCAAACTCAATGCTGCCA
CACATTTTTTTTTTTTTTGAGATGGGATCTCAGCCCTAGGCTGGCCTCAAACTCATCCTGCTGCTGACGCTTCTCTGGTCCTCCCACTAGCATC
TCCCAAGTGCTGAGATTACAGGCATGCGCCACCACACCCATCTCCTCTCAATTCTTTATGAGGAGGATACTCAGCTGCTGTTTATCTTCTCCAG
ATATCTCAAGACCTTGTGATCTTAATTTTAGCGTGTCAGTTTGTTATCAGTCGTTCCTTTTGTTGTGTTGCTTACATTTCTTTGCAAGGTTCCA
GAGCAGATAAACACAATTATAGGTCGTGTACACTGAAGAATAATGGGAGATAATTGTCATCCAGGCTTAGTGAAAACCAGGGATGTTTTAGATG
ATTAAAACAAGAGTGGAAATTTGATTTGATTGTCAAGAGCTGGCCAGGAAAGTTGGCAGTAAATATGAGCTCCTAGGCCCAAGAAGCCCTTCAG
AGCTCCCTTTTCTTTAAAGATGACAAAAACTGCCCCAGATATGGTCCCCTTGGCACCCTGCCCTAAACTCTTTATAAGACTGAAGTAGCGGCTT
CCCTGGAACTGGAGTTGAAGGAGATTGTAGGCCAACCTAGGAGTTCCTAGGTGCTAGAATGGGACCGGCTCTTAACTCCAGAGAATGGAAGCTT
AGTACGATGGTTTTGGTAGTGCCCAGGGAGGGATGAGGCATAAAGAGCAGTTGCTGCCCCTCCGAGCTGCTTTCCCCATGTGATGGCAAATGGA
ACTGCAGGGAGCAACAGAAGAAGGTACTGTGAGAAAAGGGACATTTTCTCCTAGGAAGTTTGTAAGGGGAATACAACACTTAAGTCCTCTGCCA
CTTAAATTGTGAACTTCTCCTTGTGGAAGGTCAAGGAATTTGTAAGCTGCAGCTGGTCTCAGAAGCCATCTTGTGTGCTCCCTTGAGCCATCGG
GAATGAGAAGGAAGACATCAGCTTGCTGAGTCCCTCTCCTACCTGTAAGAGTTTTACTTTTCCAGAAGTGCTTGAGGTTTCGAGCCTTGTGCCT
GCTAAGTGTGGGGCTCCATTCCTGACACTAAGTCAACTTCTGTCCCGACTAGCCACCTCATTTCAGTGAGAAAACCAAGGCCTGAGCTTTCTTA
GCCTACCTGTAGCTTCTCTTTTAGGGGGAGGGGTCATGTAGCCTACGCTGGCCTGGAGTTCACAATGTACCTGAGAATGAAAGAGCCCTGAGCC
CAGTGCCAAACAAAGTTGTCTGCCACTACACCTGGCATAGTTTCCCATAGTTTTCTGGCTAAGGAATGTTCATTGTTTGCCCAGTGGTCCTCAA
CTGGGTATGTGGTATGTGCATTAGAATCTCTTGCGAAAGTTTTTACAAATGCGGATAATCTGGGTTTTACTGCCGAACACTAAGTAAGTGTCCT
ACTGCCGAGCCATACCCCAGCTTCATACCTAAATATTTCAAATACCTTAAGTGATTTAGATCGGCTTTCTTACCCTAGTTGAGAAGTAGGATAT
TCGTACTTTTAACACGTTTAAATTTTCTTTGAATGGTTTTAGACTCTGACCTATTAGAAGTTAATGATGCTCTTCACACTTTCTTTCCGGGTGC
AGCGCTGGCAACATTTTATTTGCAGTACGTTAGAGCATGTCCTTCCTGACAGGCGTGTTTTCCGGGATAGAAAAGTAGCCTGATTATGCAACCC
ATCGAGAGACCACTAGTCCCTGAAGGGTTTTTCAGTTGGTTGTTTACGTTAGGCAGTAAAGATGTTTATGATATTCTTATTTACTTCCTTCTTT
ACTTAAAGCTCTGGGCATAATAGCTGTGTATTGCCTTGTTATATTCTGGCAATGTGGAAATGTTGCCTGGTTTATTTTAGGGGAGTAAATGGTA
TCAAATTTGCATTCTGTACTGTTCTCCCTGCACCCCCCCCCCCCCCCCCAAAAAAAAAACATTCAACAGCCACCTCTGGTTAGAAAATCAGTTA
TGGAAGAGAGTGAAGAAACCTGGCAATGTTATGGTCGGAGTAGTATTTCTACCGTCGCCTAATGTGATGCCTGCTGGCCAGTCCGCATCAGCCA
AGATCCTCTCCCTGCCCCGTGCCTCACCTGGGCAGCTGGGTGGTGTCTGTGTGCTCCCGGGGCTCATTTTCCCTCAAGCAAAAGCCCAAGTGGC
CTTGTTGGATTGGCCTGGTCCTATTTCCTCCCAATTTGGAGGAGCCCCAAGGTCTGGAGTTTACATCAACTTGGAGGCCAGGAATAAAGATGAG
AGAGATTCAGGGGAGAAAGGCTCCCTCATACTTCTATTTATAACACCTCTGCCCCTCCTGCTGGAGGCTCTACCTTTAGTACAGAGGCGCTATT
GTGTATCCTGAGGCTCTCTTTCTCCTGTACCTTTCACAGAGTTTGTTCTTGGGGAAATGAGGAGTTTAGAGAACGAGACTGAATGCATGGAGAC
TGTTGTTTGTCTGTATGTCTGTGTGTAGAGAGTAGAGGATGCTGCAGGTATGGGTTTGGGTTTGTAGTTTCCCAAAAGGTTAAGTGAGGACATT
AAGTGTGAGTTTCAGAAAACTGTATACTGGGAATGGTTGAGACTCTAATGCAGAGATTTCTGCTTTATTGTTTGAAAAGTGGCTTCAGTATGGA
ACTTAAACTGATGCTCTAATAAAATATTTTAGGATGCCAAGATATAGCAGAGCAAAGGCTGTCAAGGGATGCTCTTTAGTGCTTACACCTATTT
CCTTACTTATTTATGTGTGTATTTATGGCTCTTCCGATTGGGCTTGACGTTTATTTTAGCTAAGGAGTAGCTGTATGAGAAAGGGATGCCATTT
CATCTGTTGACGGAGGTGACATTGACCTGTGAGGTATGTAGGTACTCGGGTCTTGAGACGAGAATGTAACAGAGACGAATCAGAAGGTGGCTTA
GGGAGAAAAAGAGAAGGAGGCAAGAGAGAATGTACTGGGTAAGCCAGAGGAGTCGGAGAGCATGAGGGGCAATGCAGGCAAAGGAAACGTCAGT
TAGGGTAACGGTCACACAGACAGTTCATTCTATACTCGTCAGTCATCGATTTAGTCGTGTCTTTAAGGGCCTTTCTGGTGGATCCCTTTGAGTC
TTCCCTGGGCTACCTTCTTCCTGAGCACCCACAGCCCAGCTTACAGAGTGAGGACACTCCCCCAACTCCCTCTGTAGAAAGGCAAGTACAGGCT
GTGGCTGGCTGGAGATTGTCTGAGACTACGTGATATTAACAAGAGCTTAGCATTAACTGGAAGCTCTTTACTCCACTTGAGGGGAACTGGAGAG
GTATTTTGGAAAGAAAACAGCTTTTGAAAACAGATTCCCTTTTGTGTGTACATCTTTGCTGTTGGCATAGAAAATTTTCTGGGTTGGCCTTTTG
AGATGCCTCTCCAGAATGGTATTAGAATTCCAGAAAAACATCTGGATATTTCCCAGTTCAGACTCTTGATTTTGAAAACAGAAGTTGGGATATT
TAATTTAGGCAAAATCCCTAAGTGTATAGGCAAGGTACCTAGGAAAACCAGCATGCCTACCAGCTTTTGGAGTTTTAAGATGGAGAAGTATAGC
TTAAGGCTGAAGGAAGAAGCAGTTGTGCTACCCACAGGGTAACAGCACTACTCACAGTAACAGTGTTACTCACAGGGTAACAGCGCTACTCACA
GGGTAACAGTGTTACTCACAGGGTAACAGCGCTACTCACAGTAACAGTGTTACTCACAGGGTAACAGCGCTACTCACAGTAACAGTGTTACTCA
CAGGGTAACAGCACTACTCACAGTAACAGTGTTACTCACAGGGTAACAGCGCTACTCACAGTAACAGTGTTACTCACAGGGTAACAGCGCTACT
CACAGTAACATGTTACTCACAGGGTAACAGCGCTACTCACAGTAACAGTTACTCACAGGGTAACAGCGCTACTCACAGTAACAGTGTTACTCAC
AGGGTAACAGCGCTACTCACAGTAACAGTTACTCACAGGGTAACAGCGCTACTCACAGTAACAGTGTTACTCACAGGGTAACAGCACTACTCAC
AGTAACATGTTACTCACAGGGTAACAGCGCTACTCACAGTAACAGTGTTACTCACAGGGTAACAGCACTACTCACAGTAACATGTTACTCACAG
GGTAACAGTGCTACTCACAGTAACAGTGTTACTCACAGGGTAACAGCGCTACTCACAGTAACATGTTACTCACAGGGTAACAGCACTACTCACA
GTAACATGTTACTCACAGGGTAACAGTGCTACTCACAGTAACAGTGTTACTCACAGGGTAACAGCGCTACTCACAGGGTAACAGTGTTACTCAC
AGGGTAACAGCACTACTCACAGTAACAGTGTTACTCACAGGGTAACAGCACTACTCACGGTAACAGTATTACAGGGTAACAGCGCTACTCACAG
GGTAACAGCACTACTCACAGTAACAGTGTTACTCACAGGGTAATAGTGGTACTCACAGTAACAGCTACTCACAGGGTAACAGTTTTACTCATAC
AGCAACAGTGCTACTCACAGGGTAACAGCGCTACTCACAGGGTAACAGCGCTACTCACAGGGTAACAGTGCTACTCACAGTAACAGTGTTACTC
ATACAGCAACAGCACTACTCATACAGGAACAGCGCTACTAATACAACAACAGCGCTACTCACATAGTAACAGTGCTACTCACAGCTACTCACAC
AGCACAGAGCTACTAATGTGAGTTTTCTTTCCTAGTATAACTGTCAGTCACTTTTACCAGAGTCCATTGTAAACTTTACAGATGTGAAGCTGAG
GCAGATGATGTGTCACAGGCTTCAGTCCTAGCACTGGGGATGGATGCAGGAGGAACAGACAGGCCAGCCTAATCTACACAGTGAAATCCAGGCC
GGCCAGGGCAATAAAGAGACTTTGTCTTCTAAAAAAAAAAAAAGGTGAAGCTGGGGCTGGAGAACGGCTTAATGGTTACAGGGATTAAAGAACA
CTTAAGTTCAGATCCTCAGAAAAATGCCAGGTGGGTATGGCCTGTCTGTAATTCCAGCCCCAGAAAACAGTCGAACACTAGCCATCTTCTCAAG
CTCTGGGTTTGATTGAGAGACCCTGCCTCAATGAATAAGGTAGCAGAGTGATGTCGGATGATGATTCCTGTGATAAACCTTAGGCTTCCATGTT
CACAAATACACATGTGCGTGTGGCCCCATATGTGTATACATACATAAAGAGAAATATGAATACACACACACACACACACACACACACACACACA
CACACACACACACACTGGAAAAAAAATACAAACTGTGAAGCTGTCTGGGTAGCTCGAGTTGTAACTCCAGCCTTTGGGAGGTAGAGACATGGAG
GACTAGGAATGAGAGTCACTGTCAGCACCATCATCTCCAGCATGAGCCACAGGAGACTCAGAGAAATATGGAGCTGGGTGGCATGTGACCTGAG
CCCTCGGGGAGACTGAGGCAGGAAGATCCTGAGTTTGAGGGCAGCCTAGACTACATAGCTAGGTTCTGTCTAAACAAAACAAAACAGGAGGCTT
GTGGGGTTGTGCATGCTTACCTAACCTCAGGATTTGGAGACTGAGGCAGGAAGATTAAGAATTTGAGATCAGCATGGGCTTCCAAGTGAGACCT
GCCTCAAAACAAACAAACAAACAAACAAACCAGAGCAAAGTGAAACAATTTTAAAAGTCTCTGAGTAGTTACTGTCATTGCTAAGTCACACAGC
TGCCCCCATGTTACTCACAACTGCATAAAGCACACTGAAAAAGCCTGAAACTCCTTGGAGTCTTGGCTGCTGAGGTAACTGGGCCATGGAGAAG
AATGTTCAGATGCTTGGGTGAACTGGAAAAGATTGGGACTTTGAATTTGAGCAGGTATAGGCTTTGATTCCGGCTTTACAAGCTGTTTAACCCC
AGGCAGTGAGCTGCATGACTCTGCAGAGGGAAAAAAATGTTACAGAGTTGCTATGGGAGTCAAGTAGTAGCCCCGAGAGATACAGAAAACACTC
AACAAATGCATTTTTAAGAGATTTACTTCTTTTAGTTTCTTTTACATGAGGGTTTTGCCTGCCCATACTTGCATGTGGGTGCTGGGAATTTTAA
ACCCACATCTTCTGCAGGAGCCTTGAGCCATCTCGCCAGGCCCAATGGATACATTCTTTAACCTGGTCTGTGGCCAAAGGGAGATGTAAGGATG
GAGGGATAAGACACGTTAAGCACGTTTTACCAAGTATGTAGCCTAGGTTGCTATGAATGTGGCCCAACACATTCGGGCACAATCTTATCACAGT
GGTGGAAGTTAGACACTCCTGTAAGGTTGGGAGCCCTCGTTTCCACTGGAGACTTCAGTGATTTTTGGAGAGGAAACCCAGTCACAGAGAAGAG
TGGAGGTGGAGGGAGTAAGGAGGCTGCCCTTTTATTGACTACTATACTGCTGTATCTAGCATCCCTTTACACTAAGATAGCAATAAGGGACGGC
AGGGATCGAGGGATGCTCTGTGTCAAGTGCATAGTTTTAGGTAGTTGTATCATAAGTTCAACATTTTTAAAAGATTCAGTTTATTTTTATAGAC
ATGTGCACATGTGTGATGGGGAGGTCAGATGAGGGAGTTGAACCTCCTGGAGCTGAACTTGTAGGCACTTATGGCCTCCTGTGTGGGAGCTGGG
AACTGAACGCATGTCCTCTCTAAGAGCCATTCAGCCATCTCTCCACCACCACAAATTCCAAAATTTCTCACTGTTTTTAACATCTATATAAAAT
TTATTTAGATCTATGCCAATTTAAAACATTGCTTTGACTATTGGCTACTAAGCTGAACATTTTGTTACGTTTTTGTCTATAGTTTTATTTTTAT
ATCCAGATTTTCTGTTGTTGTCTGCTTATCTTTTATGTCGTCTGCTTATCTTTATGTTAACTTTTGTAAGCTCTCCAGTAGTGTGTCTGTGTGC
ACGTACATGTGTGTGTACGTGAGTGTATGCACATGTTTTTGTGGGTGAGAGTGCACATATGCTCATATATGTGGAAGCCAGGAGACAACTCCAC
GTGTCAATTCCTCAGGCAAATATCCACTCTTTTGGAGACAAGGTCTCATTGTCCTGAGCTTATGTCAAGTAAGGCTAGCCCTGAGATTACAAGC
TGCTACTATGCCTGGCTTTTCCCTTAGGTTCTGGGTTTGACTCAGCTCTTCAGTTTGTAGTGAATCCCTTCCTGCTCTGCTTCCCTGTGCTCTG
GTCACAGGTATACTCAGCCTGCTAACTTCTCTGTGATTTAATAACTTTACTTAAGATTTTCTTTAATTGTGTGCGTGTGCGTCTGAGACCCCGT
CTAGCTCTGGACTTGCCTGTCATGGTGAGTTAAGCACTTCTCTTGACTGCCCAGCACTTCACTTGCTTTATCCGTTCTTACTGAGAAACTGCAG
CAGTTTTATTTTGGCTCCTTCTCAAGAACTGTGACTTGTTAATTTTTTTACTTTTATAGAATTTTTGTTCCTTTCTGATTGGCATTGTGTTAGC
AGTTTTGTCTGTAATGTGGGCTTTTCAGTGGGCCTCTTTTAGATGTGTATGTGTGAGAGACAGGCACCTTACTTCCAAGTCAGTCCTCAGTCAG
ACTCTTTTTGATTTATAATGTATTTTAAAAGCTTTCCCCCTCCCCTTTTTATTTCTTTTTTTAAAATTTATTTTATTTGTTTACATTCCAAATG
TTGCCCTCTCTTTTATTTCTTAAAATTAATTTTCAAGTTAGCACACACGGTAATGGGGTCATTGTCTGTTGTCATCTTTCTGCAGATCTGTCAT
TATATTTTGTTTTTCCATGTGCCCTCTGCCCTCCTCCTGCCTGGTCCCTTCTTGCAATATCTTTTTATTTATTTTAGCTCTAGGAAATTATCCA
TTTTACCTTTAATAATTGCTTCTGATTCATTAGTTCTGTCTTGAGGGTGAGCTGCACAATCTCTTTATGGCTTCTCTTCATTTCATTTGTTTAT
GTAAAGTCATAGCAACCAAGTTGTGGCGTAAATCATCTGCAGTGGTCATCCTAGGTGTGTGTTCTGCTCTCTGTTTGCTGCTGGCTTGGTTTAG
TTCTCTCTGGTTCTGTTGTCTGACTTCTTAGACCGTTCATTTTCACCTGTAAAGTCTTTCTATTCTTGTTAGTAGTTAAGAGGGCCTTCATTAG
TTCATGAAGAGCAGAGGGGTGCATAGCAGTCCCCCCAGAGGTGGAGAAAAGGAGGCTCATGCGAGGCGTGGGTTTGACATTTTACAGTTCTCCT
TACAGACAGTGTGGAGGTGGACGTGACCACACACCAGTGTTAACCCGGCGGCTGACTGTACAGACTGTTGACCAGTCAAGATTGAGAAAGTCTT
TGAGAGCTAAGCAGCCTGTTCATTTGACCTAATTTGGTATATATTTCTCCTGGATTTAGAGATCAAAGCAGTGATTGTGCTCATGTATTTTAAA
CCCCATGAGACAAAATGGTGAAAATGTATTTGAAGTCTTGAGTTAGCTTATTCTAGGAATTTCATTTCCTGGTCTTGCTGGGTGATCTGTTTCT
GATTCTTTATAATTTATTCTGCTTCACTATGAACTAGTTCAGAGATACCTGTTGGTAGATAGCAGGGCCAGTTTTGAGTACTAGTAAATGATAT
GATATTAGATGGACCTCTGGTTGCTTCTAATTGGAGTTGCCTAAAATAACCCTGTGACAAGTATTTGTGTAAATAAAGTGCTGGTAGGCAATGT
GAGACCTTCAGGAAGTGATTCATATCACTCTGCTGTTTGTTTAATTTGGGACTCCGTTGCTTAGAGACCATATCTTTGAGGAGGCTGAGTGTGA
AATACTAGCTGAATGCCAAGGGCCAGGGGCTACCTGATCAAATTAATCACAGCCCTGCTTCTTCACTTTAGTTTTAGAAGGCTCCTTATAGTGG
CTTGGCAGTGTTTTTATTACAAAAATTAATATAAAAAACATATAAATGAAGGATCTGGCTAATGATTTAATTTGAAATGTAAATATTTACTTAA
ATTTGAATTTTCAGGAAACATTTTTTTTTGATAGAATTGGTTATTTCTACCAGGCATTATATATATATATATATATATATATATATATATATAT
ATATATTAAACAGACTCTTTCCTTGTACCCCTCCCATACCCATTTCTTGAGAATAGACATTGTTTAGATCTGGAAATCCCCTACTCTCCCCTTC
TCCTTTCTCCCCTGAGGGCCTATAAAAACTGGGACCTCTTTCCCCTCGAGGTCGACTCCTCTACCTCTGCATGGCATATGAGTCGTCCCCAGAG
CTCTGGCTTTCCCCGAATAAAGCCTCATGTGGTTTGCATCAAGCTTGGTCTATCGTGAGTTCTTGGGTGTCCGCTATTGTCCTGAGGCCTGAGC
GAGGGGCTCCTCTTGGAGTCTTTCAGTTCCAGACAACTGGTGTCAGCTGTCATATGGCCGAGGACTGGAGCTGTGTCTGCTCATTAAAGTCTCA
TCCACCCTCAAATGTCCAGTTTGTCTTTTGGTTTGTTTTTTATATTGAGAACTGAACTGAGAGTCTCACAGATGCTGGGCAGATTACCTGCTGC
TAAAACACACCCCAAATCCCAGTGATTAGACAAACCAGTTTGAAGCAGGGTCTCCTTAAGTCGTCTGGGTAGCTTCTGGTTTCAGTCCGCTGTG
GTTCAGGAGTAGGTTGGGTTTACAGGCATGGTCCACCACACCCATTTCGTCACGTATTTGTGCTTTTAACTGGATTGTTGGGCTATTCCTCACC
GTTTTTTTTTTTTTTTTCCTACGTTCTAACCTGGTCTCTGCCTTTGGGTTTAGCTTGTGTCCCACCAGACTCTCCTTTGCTAAATAGTGACCTC
ATGCAGACAGGAGTGGCAAGTTGGATTGGAAATTGCCTTACATCGTTTTGGCAAGAGCCGTGTTTGGGGGGTGTGGGTGGAGGAAGGACTTGTC
TGAAGCCCTCTATTTCTTCTTTACTGAGAATGCTTTCCTGTTGACTCACTGGAAATCATAACGAAAAGCAGAGGAACACTGGAAACGCAGCTGC
CAGCCCAGGGCTGTGCTGCTGCTGGGCAGCTGCTGAGGGCCTGGGGAGACACCAGGCGCCAACATTGGTGAGATWTTTGAGGAGAGAAAATAGA
AGGGGATTCTGGTATTATTGTGAAAGGTAATTTTTCAAAAAGTATAGTTATGGCCCAGATTGAACTTTTCTTTTTTAATTCAAAACCAGAGTGT
GCCTGGCTGTGGTACATAATTCAGAGGGGATGGGAGATCCAGAGAAGAAAAAGCCCACCTGTCTCCTCGGTCCTGTTTCTCCTACCCTGTGCCA
TCTTTTCCAGTTAGATGGCTGTTAGCATTGTCCCCTGTGTATGCCATATGTATTATAAATATTTATTCCACGTTACCCTTTACACTTTTATTTT
TGTAAGGTGTTTATAGTAATAGCATCTTAAGACATGTTTGAATTTATTAATCTTATTTTTTAGCTTCTGAAGTCCTCTTCAGGCCATTTTCATA
CAGTCCTAGTATATATATGAAATTATAGAAAATCATTTGAGTTTGGAGAGATGGCTAAACAGTTAAGAGCACTAATTGTTCTTCCATAGGACCT
GAATTTGATTCCTGGCACCCATACTGGGTGGCTCATAGTTGTCTTAATTCTACTTTCCTGGGCATGACTCCTTTGGCCTCTTAGATGCCTGTAA
TGATCTTGCCCATGTGTACTTCACAATTAATGAAAGCAAAATAATAGCAAAAAGCAAAAAGAAAAAAAAAAAAAAAAGGAGGGGGGTGTTGTTC
AATGTAGCCATGTTGGGAAGGGAGACAAACAAAGCTAGCACCACGGTTCCTAAAGTCCTAAAGTCCGTCCTCAAAGGGTCTGCCCTAAAGGTAG
GAAGCAAGATACATAACTAAGCAGTCCAATAAGATGTGGTCCAGCCCACCATCTCAGCTCCAAGCCTCCTGCAGGGTCATCTGAGGGGCGCCAG
ATATTTTTCCTGGAAACATGTGGATGCCCCCAGGGTGGCATTCTTTCCTCTTTTAGCAGGAAGGAGTCCTGGGAACTCTCCAGTTTCTTGGGGT
CCATTCTTTCAGTAATCTGACACCCAAAGATGGAGCACACACCTCTCTTTAAGAGCATATCCTGCTAAACAGGTTCATTACATACTCATACATA
CACACAGCTAAAAATCACCCAAAACTAAGTCTTAAAAATACTCCCTTATGGTTTCCTCCCCTTCTCCCTTCAGTAATGGAGCTTGAACACAGGG
CCTCGACCATGCTAGGCAAGTATTCTGCTGGGCTTTACTTCCAGGCGAGCCCCTGCCTAATAGTGTTTTAAAATATATATATACATGTATATGT
AGCATCACTTAATATTTACATGTGTATTATAATATATTAAATATGAATGTATGTAATATATATATACATACACACATGTAACATGTAAGTGATA
AAACTGGACATGTGTATATATGTGTACACACATATATGCAGCGTTTTAACATTTCATTTAACTCTTTGATTCATTTAGGATTTTGGCATGAGGA
CTTGGGTAGATATCTTATTTATTTTGTTTTGTTTGAGGCTTGCTCTCACTATGTAGCCCTGGGTGGCCTAAAACTAACTCACTGTGTGGACCAG
GCTGGCCTTGCACTCAGAGATTCACCTGCCTCTGCCTCCTGAATGCTGAGATCAACAGTGTGTACCCGTGTGTGTGTGTGTGTGTGTGTGAGAG
AGAGAGTGTGTGTGTGTGTGTGTGTGAGAGAGAGAGAGAGAGAGAGAGGATCTAAATGAAAGACTTTCACTGTTTCGGAAATATAATCGCTGGG
CTGTTGCCCATCATCTTTAGGACACTGTTTTTGTCAATTACATGATCTCATGTAGCCAAGGCTACTCCTGGTTCTCCTACTTGTACCACCTAAG
CACTGGAACTACACGTGTGTGCCGTATGCCTAGCTTCTTGAGGCACTGTAAAGGACAGAGTTGTGAGTTCTAACAGTTAGGATTGCAAATGTAC
TAATCCTAATTATTTCAAACAAAAAGAAAACATATCACAAAATCTGATCTAGTGAACTAATTTCTGACAAAATACAGGAGTGGCAAAATTCCAG
GGTACATTATCAGATAGCTGTCTTCTGAGGACTTAGTAAGACCAGAATGACATGATGCTAGGAGCCACCCTGGCTCCTCACTGTGGACTCCTTC
GGAGAGCCATGCTGCAGAAACGTCTACTCTTGAACAGGGTCACCACATTGGTGAGTCAGAAAAACATGTAAAGTCTAGAGTGTAGCAAGGCATT
TGCAAGTGTGTCTTGTGATACTTTGCAGAGAGATTGGTTAAATATGAATTAGATTTCAGTGTAATTAAGATGATTTATAGCTGCCTCAGTAAAC
CAGATTTAAAAGGGCCCTTAGAGGGGCGTGGCTGCTAGAGGGATATAGGTTTCTTTTAGGGTAATGAAAGTTTCTGGGACTATATTGTAATAGT
GGTTGTACAACATAGCGAGTATAACAAACCAAAAACACCACATTGCTTATGGTTAAAAAGGGTGAACTTGCAAACTCTGGCTCAGAAAAAGAGC
GATTGGTTTTGTTTGGTTTTCCTTTTGAGATCTGTGTAGCTCACACCAGTCTTCCATGTGTTGTGCACTGAGGATGGGTGACCTTGGCCTTCTG
GTTTTTCTGTCACTGTCTCTCCAGTGATTGAGAGCTAAGCAGGCCTTCATGTGTGTCAGACCAGCAGTCTACCAACTGAGCTGCGTACCCAGCC
CCGCCTTGTTTGTTCCTTTGGTTTTTTTGAGATAGCATCCTATGTAACCCAGGCTGGCCTCAGACTCCTTGTACACTTAGAGGATGACCTTGAA
CTCCCGATCCTCTTTCCTCTACCTTCCTAGTTCAGGAATTACAGGTACGACCCACCTGTCATCCCTCCCAGCTCTAGTTGCTGGATTTTAACGC
AACGGAAGCTCTTTAGGGCCTCTTCTCACAAGGGATGTGCCAGTCAAATTTGGACTGCTAGAGAAGTGGTTAACACAACAAACTGTGTTTAAAA
TACACCAGGTTACCACAGTGTAATAGGATAATGGTTTACGTTGTGGTAGAAAAGGTCTAACTGCAGAGTAAAAGAGAAGCAGCATTAGTAGGAA
AAGCAAACGACATCAGAGATACTTGTTAACCATTAAGTCTGGTGACCTGCATTTGACCCTTGGAAGCACTGTAAAGGTGGAAGGAGAGAGCTGC
CCCAGACCTCCCATGTGCATTGTGGCCTGGGATACACATACGTACAATAATTGCAAATTAAAAACAAATCACGAGGTGGACAGGACAGCACCCA
AGGGATGATGTTTGAGGTTGACCTCTGGCCTTCACACACATGTGCAAGCGTGTCCACATCGACCCTTTCTAAGAGCACATGCACAAAGTCGGAG
CCACTGTGGCAACCCACGTTGTAGGAGACTTTCAAACACAGATCCACAGTTCCAGGGAAGCTTCTTCCTGTGCTCTAACCCATTTCTGCAGTTA
MOUSE SEQUENCE—mRNA
AGCGACAGCCCGCCCACTGGGCAGGAGTTACTGCTGCTACGGTCGCCCGCGTCTTCAAGGTCTCTGCGCTTCCTCACCGGAGACCTGGACTCGG
CCGCCATGTCCGCCGCGGAGGAGGTTGATGGCCTGGGTGTGGTCCGGCCGCACTATGGCTCCGTCCTGGATAATGAGAGGCTCACTGCAGAGGA
GATGGATGAGCGGAGACGGCAGAACGTGGCTTATGAATACCTTTGTCATCTGGAAGAAGCAAAGAGGTGGATGGAAGCATGCCTAGGTGAGGAC
CTGCCGCCCACCACAGAGCTAGAGGAGGGCCTTAGAAACGGAGTCTACCTTGCCAAGCTAGGGAACTTCTTCTCTCCCAAAGTGGTGTCCCTGA
AGAAAATCTATGATCGAGAACAGACCAGATACAAGCCTACCGGCCTCCACTTCAGACACACGGATAATGTGATTCAGTGGCTGAATGCCATGGA
TGAGATTGGGTTGCCTAAGATTTTTTACCCAGAAACCACAGATATCTATGACCGGAAGAACATGCCAAGATGCATCTACTGTATCCACGCCCTC
AGTTTGTACCTGTTCAAACTGGGCCTGGCTCCTCAGATTCAAGACCTGTATGGAAAGGTTGATTTCACAGAAGAAGAAATCAACAACATGAAGA
TCGAGCTGGAGAAGTACGGGATCCAGATGCCTGCCTTCAGCAAGATCGGGCGCATCCTGGCTAATGAGCTCTCAGTGGATGAAGCTGCGCTACA
TGCTGCTGTTATTGCTATTAATGAAGCGATTGATCGCAGAGTTGCAGCTGACACTTTTACGGCTCTAAAAAACCCCAATGCCATGCTCGTCAAT
CTTGAAGAAGGCCTGGCTCCCACGTACCAAGACGTGCTTTACCAGGCCAAGCACGACAAGATGACAAACGCTAAAAACASGACGGAAAACTCTG
ACACAGAAACGGACGTTTATGAGGAGCTGCTCACACAAGCTGAAATCCAACGGAATGTAAACAAAGTCAACACATCTTCTGCCCTGGCCAACAT
CAGCCTGGCTTTAGAGCAGGGCTGTGCAGTGACCCTGCTCAAGGCTCTGCAGTCACTGGCTCTGGGCCTCCGAGGGCTGCAGACCCAGAACAGC
GACTGGTACATGAAGCAGCTACAGAGTGATCTGCAGCAAAAGAGACAGAGTGGCCAGACTGACCCCCTGCAGAAGGAGGAGGTACAGGCCGGAG
TGGATGCTGCCAACAGTGCTGCCCAGCAGTACCAACGACGGTTGGCAGCAGTGGCAGCAATCAACGCTGCCATCCAGAAGGGCATCGCTGAGAA
GACCGTGTTGGAGCTAATCAATCCTGAAGCCCAGCTGCCCCAGGTGTATCCATTTGCACCTCATCTCTATCAGAAGGAGTTGGCCACCCTGCAG
CAGCAGAGCCCTGAGCATAGCCTCACCCATCCTGAGCTCACTGTTGCTGTCGAGATGCTGTCATCCGTGGCCCTCATCAACAGGGCGCTGGAGT
CAGGAGACATGACCACTGTGTGGAAGCAGCTGAGCAGCTCAGTTACGGGCCTTACCAACATCGAGGAAGAAAACTGTCAAAGGTATCTCGATGA
GCTGATGAAGCTGAAGGCTCAGGCACATGCCGAGAATAATGCATTTATTACATGGAATGACATCCAGCCGTGTGTGGACCATGTGAACCTGGTG
GTCCATGAGGAGCATGAGCGGATTTTGGCCATCGGCTTGATTAATGAAGCCCTGGATGAAGGGGACGCTCAGAAGACTCTGCAGGCCCTGCAGA
TCCCTGCAGCCAAGCTCGAGGCCGTCCTTGCAGAAGTGGCACAGCACTATCAAGACACGCTGATCAGAGCAAAGAGAGAAAAGGCCCAGGAAAC
ACAGGATGAGTCAGCTGTGTTATGGTTGGATGAAATTCAACGTGGAATCTGGCAGTCCAACAAAGACACCCAAGAGGCCCAGACGTTTGCCTTA
GGAATCTCTGCCATCAATGAAGCAGTAGACAGCGGTGATGTTGGCAGAACCCTGAGTGCCCTACGTTCTCCCGATGTTGGCTTATATGGAGTGA
TCCCCGAATGTGGGGAAACGTACCAGAGTCACCTTGCTGAAGCCAAGAAGAAGAGACTGGCAGCAGGAGATAATAACAGCAAGTGGGTGAAGCA
CTGCGTGAAAGGCGGGTACCATTACTACCACAACCTGGAGACGCAAGCAGGAGGATGGGCTGAGCCCCCAGACTTTGTGCAGAATTCTGTGCAG
CTTTCTCGAGAGGAGATCCAGAGCTCCATCTCTGGAGTAACCGCTGCATATAACCGAGAGCAGCTTTGGCTGGCCAACGAAGGCTTGATCACCA
AGCTGCAAGCCTGCTGCCGTGGGTACCTCGTTCGACAGGAATTCCGATCCCGGATGAATTTTCTGAAGAAACAGATCCCTGCCATCACCTGCAT
TCAGTCACAGTGGAGAGGATACAACAAAAAGAAGGCATATCAAGATCGGCTGGCTTACCTGCACTCCCATAAAGACGAAGTTGTGAAGATTCAG
TCCCTTGCCACGATGCATCAAGCTCGAAAGCGCTATAGAGATCGCCTACAGTATTTCCGAGACCATATAAATGACATTATCAAAATCCAGGCTT
TCATTCGGGCCAACAAAGCTCGTGATGACTACAAGACTCTCATCAATGCTGAGGACCCGCCTATGATTGTGGTCCGAAAGTTTGTCCACCTCCT
GGACCAAAGTGATCAGGACTTCCAGGAGGAACTTGATCTCATCAACATGCGCGAGGAGGTCATCACCCTCATCCGTTCCAACCAGCAGCTGGAG
AACGACCTCAACCTCATGGATATCAAAATCGGACTGCTGGTGAAGAACAAGATCACGCTGCAGGATGTGGTTTCCCATAGTAAAAAACTTACCA
AAAAAAATAAGGAACAGCTGTCCGACATGATGATGATAAACAAGCAGAAGGGCGGGCTCAAGGCTTTGAGCAAAGAGAAGAGGGAGAAGCTGGA
GGCCTATCAGCATCTCTTTTATCTCCTGCAGACCAACCCTACCTATCTGGCCAAGCTGATCTTTCAGATGCCACAAAACAAGTCCACCAAATTC
ATGGACTCTGTGATCTTCACGCTGTACAACTATGCATCTAACCAGCGGGAGGAGTACCTGCTGCTGCGGCTCTTCCAGACAGCTCTGCAGGAGG
AGATCAAGTCAAAGGTGGATCAGATTCAAGAAATCGTGACAGGAAACCCTACGGTTATTAAGATGGTTGTAAGTTTCAACCGTGGTGCCCGGGG
CCAGAATGCCCTCCGGCAGATCTTGGCCCCTGTCGTGAAGGAAATTATGGATGACAAGTCTCTCAACATCAAAACCGACCCTGTGGATATTTAC
AAGTCTTGGGTTATCAGATGGAGTCGCAGACAGGAGAGGCGAGCAAACTGCCCTAAAGATGTGACCCCTGAACAAGCCTTGTCTCATGAAGAAG
TGAAGACGAGGTTAGACAACTCCATCAGGAACATGAGGGCTGTGACAGACAAGTTCCTCTCAGCCATCGTCAGCTCTGTGGACAAAATCCCTTA
TGGGATGCGATTCATTGCCAAAGTCCTGAAGGATTCACTTCACGAGAAGTTCCCTGACGCTGGTGAGGACGAGCTGCTGAAGATTATCGGTAAC
CTGCTTTACTACCCATACATGAACCCAGCCATCGTCGCTCCCGATGCCTTCGACATCATTGACCTGTCAGCAGGGGGCCAGCTCACCACAGACC
AGCGCAGAAACCTGGGCTCCATTGCCAAGATGCTCCAGCACGCGGCGTCCAACAAGATGTTTCTGGGCGATAATGCCCACTTAAGCATCATTAA
TGAGTATCTCTCGCAGTCCTACCAGAAATTCAGACGGTTTTTCCAATTGGCTTGTGACGTCCCAGAGCTGCAGGATAAATTTAACGTGGATGAG
TACTCTGACCTAGTCACCCTCACTAAGCCAGTTATCTACATCTCCATTGGCGAAATCATCAACACCCACACTCTCCTGTTGGACCATCAGGATG
CCATTGCTCCAGAGCATAACGACCCCATCCACGAACTTCTGGACGACCTTGGGGAGGTGCCCACCATTGAGTCCCTTATAGGAGAAAGCTGTGG
CAATTCAAACGACCCCAACAAGGAGGCTCTGGCTAAGACGGAAGTGTCTCTCACGTTGACCAACAAGTTTGACGTGCCTGGTGACGAGAACGCA
GAGATGGACGCTCGGACCATCTTACTGAATACAAAACGTTTAATTGTGGATGTCATCCGATTCCAGCCAGGAGAGACCTTGACTGAAATTCTAG
AAACCCCAGCCACCAATGAACAGGAAGCTGAACATCAGAGGGCCATGCAGAGACGGGCTATCCGCGATCCCAAAACCCCTGACAAGATGAAAAA
ATCAAAGCCCATGAAGGAGGATAACAACCTCAGCCTCCAGGAGAAGAAAGAGAAGATCCAGACTGGCCTAAAGAAGCTAACGGAGCTTGGGACG
GTGGACCCAAAGAACAGATACCAGGAACTCATCAACGACATTGCCAAGGATATCCGGAATCAGCGGAGATACAGGCAGAGGAGGAAACCTGAAT
TGGTAAAACTGCAGCAGACGTACTCGGCGCTGAACTCTAAGGCCACCTTTTACGGCGAGCAGGTGGACTACTACAAGAGCTACATCAAAACCTG
CTTGGATAACTTGGCCAGCAAGGGCAAGGTCTCCAAAAAGCCTAGGGAAATGAAAGGCAAGAAAAGCAAAAAGATTTCTCTGAAGTACACAGCA
GCGAGGCTGCATGAGAAGGGCGTCCTTCTGGAGATTGAAGACCTTCAGGCAAACCAATTTAAAAATGTTATCTTCGAAATTGGTCCAACAGAAG
AAGTTGGAGACTTTGAAGTAAAAGCCAAGTTCATGGGAGTTCAGATGGAGACTTTCATGTTGCATTATCAGGACTTGCTGCAGCTACAGTATGA
AGGAGTTGCAGTTATGAAATTATTTGATAGAGCTAAAGTGAATGTCAACCTCCTGATCTTCCTTCTCAACAAAAAGTTCTATGGGAAGTAATGG
GTCGCTCGCCGCCCAGAAGAGTTAACAAAAAACGGCAGCACCTCACGGCTGTCTTTCTAGGATCCTCCATTATTCCTTAGAAGCAAGGACCTGG
TCCAGTGGCGGTGCCTCAGTTCACACTCCCTCTGACGGACGGACGGACGTCAGTGCCCTCTCCCTTCTCCTTGTGAAGCCATAAGCCTGACTTC
CCCTCAGCCCTGCTCCCTCATTTTCTCTCATCATGTAGGAAAGAGTGGGACCCCCTCCCAACCCCTTTCTGTTAAAGCCTCCTAAGTGGTCATT
ACTACTTTGTAGACATTGGTATTTGTTTTACTTAGCAATAAGAATGGTGAAATTCAAATTCTCGTTTAGAAGTGAAAGCTGTCAGGTTGATAGC
AAGCATGCACATCAAATTCACCAGAAGTACAACCCATCGGCGCAGTCAGAGGATGGAGTCTGATGTCTTCCGGCTGCTCCCTTTGTGGGCAGAG
CTAATCCAACTTACTGTTCTCTGTTCAAATGAAGCAGCTTCAGGAGAAAAAGAGCAATTACTTTTGAATTCTCCAATAAATCTGGACAATGCCG
TTAGCGAAGAGCCCTTCCTGGTGAAGGCAAACCCATGGTCAGAGCAGGCCATTTAGAGACTGAGTGGGCGGGGCACTTACCATCCCTTCCACAA
AGGATAGCTTCCCCCAGCTCAAAATGTAACTGTTTCTAAACTGTATTCCAAGAGTGCCTTACCAAAGTATAAATTTATTTCTTAAAATGTGAGT
AATAGGAATTTTAAAGATTTATATAATGCTTTTGAACACTCTGAGAAAGGGTTGTTTTTTTTCATTGGGTTAATCTGTATATCTGAATTCTTGA
AGCTTTTCTCTAGCCTACAGTAGGGTCGATCTGCCGAGCTGCTGAATAACCCTGCTTGATTTTAGCAACAAGAGACAATCCATGCCATGCTTTT
ATCCTGAGAAACCTGGACCCTTAGGCTCTGACTAACAAAGACAGTTCAGCTCCAGACACTGAACTTGGAATCCTTTGTCTTTGGATTAAAAGTC
ATAAACTTAAAAAA
MOUSE SEQUENCE—CODING
ATGTCCGCCGCGGAGGAGGTTGATGGCCTGGGTGTGGTCCGGCCGCACTATGGCTCCGTCCTGGATAATGAGAGGCTCACTGCAGAGGAGATGG
ATGAGCGGAGACGGCAGAACGTGGCTTATGAATACCTTTGTCATCTGGAAGAAGCAAAGAGGTGGATGGAAGCATGCCTAGGTGAGGACCTGCC
GCCCACCACAGAGCTAGAGGAGGGCCTTAGAAACGGAGTCTACCTTGCCAAGCTAGGGAACTTCTTCTCTCCCAAAGTGGTGTCCCTGAAGAAA
ATCTATGATCGAGAACAGACCAGATACAAGGCTACCGGCCTCCACTTCAGACACACGGATAATGTGATTCAGTGGCTGAATGCCATGGATGAGA
TTGGGTTGCCTAAGATTTTTTACCCAGAAACCACAGATATCTATGACCGGAAGAACATGCCAAGATGCATCTACTGTATCCACGCCCTCAGTTT
GTACCTGTTCAAACTGGGCCTGGCTCCTCAGATTCAAGACCTGTATGGAAAGGTTGATTTCACAGAAGAAGAAATCAACAACATGAAGATCGAG
CTGGAGAAGTACGGGATCCAGATGCCTGCCTTCAGCAAGATCGGGGGCATCCTGGCTAATGAGCTCTCAGTGGATGAAGCTGCGCTACATGCTG
CTGTTATTGCTATTAATGAAGCGATTGATCGCAGAGTTGCAGCTGACACTTTTACGGCTCTAAAAAACCCCAATGCCATGCTCGTCAATCTTGA
AGAAGGCCTGGCTCCCACGTACCAAGACGTGCTTTACCAGGCCAAGCAGGACAAGATGACAAACGCTAAAAACAGGACGGAAAACTCTGACAGA
GAAAGGGACGTTTATGAGGAGCTGCTCACACAAGCTGAAATCCAAGGGAATGTAAACAAAGTCAACACATCTTCTGCCCTGGCCAACATCAGCC
TGGCTTTAGAGCAGGGCTGTGCAGTGACCCTGCTCAAGGCTCTGCAGTCACTGGCTCTGGGCCTCCGAGGGCTGCAGACCCAGAACAGCGACTG
GTACATGAAGCAGCTACAGAGTGATCTGCAGCAAAGAGACAGAGTGGCCAGACTGACCCCCTGCAGAAGGAGGAGGTAAAGAACCGGAGTGGAT
GCTGCCAACAGTGCTGCCCAGCAGTACCAACGACGGTTGGCAGCAGTGGCAGCAATCAACGCTGCCATCCAGAAGGGCATCGCTGAGAAGACCG
TGTTGGAGCTAATGAATCCTGAAGCCCAGCTGCCCCAGGTGTATCCATTTGCAGCTGATCTCTATCAGAAGGAGTTGGCCACCCTGCAGCAGCA
GAGCCCTGAGCATAGCCTCACCCATCCTGAGCTCACTGTTGCTGTGGAGATGCTGTCATCCGTGGCCCTAATCAACAGGGCGCTGCAGTAAGGA
GACATGACCACTGTGTGGAAGCAGCTGAGCAGCTCAGTTACGGGCCTTACCAACATCGAGGAAGAAAACTGTCAAAGGTATCTCGATGAGCTGA
TGAAGCTGAAGGCTCAGGCACATGCCGAGAATAATGCATTTATTACATGGAATGACATCCAGGCGTGTGTGGACCATGTGAACCTGGTGGTCCA
TGAGGAGCATGAGCGGATTTTGGCCATCGGCTTGATTAATGAAGCCCTGGATGAAGGGGACGCTCAGAAGACTCTGCAGGCCCTGCAGATCCCT
GCAGCCAAGCTCGAGGGCGTCCTTGCAGAAGTGGCACAGCACTATCAAGACACGCTGATCAGAGCAAAGAGAGAAAAGGCCCAGGAAACACAGG
ATGAGTCAGCTGTGTTATGGTTGGATGAAATTCAAGGTGGAATCTGGCAGTCCAACAAAGACACCCAAGAGGCCCAGAGGTTTGCCTTAGGAAT
CTCTGCCATCAATGAAGCAGTAGACAGCGGTGATGTTGGCAGAACCCTGAGTGCCCTACGTTCTCCCGATGTTGGCTTATATGGAGTGATCCCC
GAATGTGGGGAAACGTACCAGAGTGACCTTGCTGAAGCCAAGAAGAAGAGACTGGCAGCAGGAGATAATAACAGCAAGTGGGTGAAGCACTGGG
TGAAAGGCGGGTACCATTACTACCACAACCTGGAGACGCAAGCAGGAGGATGGGCTGAGCCCCCAGACTTTGTGCAGAATTCTGTGCAGCTTTC
TCGAGAGGAGATCCAGAGCTCCATCTCTGGAGTAACCGCTGCATATAACCGAGAGCAGCTTTGGCTGCCCAACGAAGGCTTGATCACCAAGCTG
CAAGCCTGCTGCCGTGGGTACCTCGTTCGACAGGAATTCCGATCCCGGATGAATTTTCTGAAGAAACAGATCCCTGCCATCACCTGCATTCAGT
CACAGTGGAGAGGATACAAACAGAAGAAGGCATATCAAGATCGGCTGGCTTACCTGCACTCCCATAAAGACGAAGTTGTGAAGATTCAGTCCCT
TGCCAGGATGCATCAAGCTCGAAAGCGCTATAGAGATCGCCTACAGTATTTCCGAGACCATATAAATGACATTATCAAAATCCAGGCTTTCATT
CGGGCCAACAAAGCTCGTGATGACTACAAGACTCTCATCAATGCTGAGGACCCGCCTATGATTGTGGTCCGAAAGTTTGTCCACCTCCTGGACC
AAAGTGATCAGGACTTCCAGGAGGAACTTGATCTCATGAAGATGCGCGAGGAGGTCATCACCCTCATCCGTTCCAACCAGCAGCTGGAGAACGA
CCTCAACCTCATGGATATCAAAATCGGACTGCTGGTGAAGAACAAGATCACGCTGCAGGATGTGGTTTCCCATAGTAAAAAACTTACCAAAAAA
AATAAGGAACAGCTGTCCGACATGATGATGATAAACAAGCAGAAGGGCGGGCTCAAGGCTTTGAGCAAAGAGAAGAGGGAGAAGCTGGAGGCCT
ATCAGCATCTCTTTTATCTCCTGCAGACCAACCCTACCTATCTGGCCAAGCTGATCTTTCAGATGCCACAAAACAAGTCCACCAAATTCATGGA
CTCTGTGATCTTCACGCTGTACAACTATGCATCTAACCAGCGGGAGGAGTACCTGCTCCTGCGGCTCTTCCAGACAGCTCTGCAGGAGGAGATC
AAGTCAAAGGTGGATCAGATTCAAGAAATCGTGACAGGAAACCCTACGGTTATTAAGATGGTTGTAAGTTTCAACCGTGGTGCCCGGGGCCAGA
ATGCCCTCCGGCAGATCTTGGCCCCTGTCGTGAAGGAAATTATGGATGACAAGTCTCTCAACATCAAAACCGACCCTGTGGATATTTACAAGTC
TTGGGTTAATCAGATGGAGTCGCAGACAGGAGAGGCGAGCAAACTGCCCTATGATGTGACCCCTGAACAAGCCTTGTCTCATGAAGAAGTGAAG
ACGAGGTTAGACAACTCCATCAGGAACATGAGGGCTGTGACAGACAAGTTCCTCTCAGCCATCGTCAGCTCTGTGGACAAAATCCCTTATGGGA
TGCGATTCATTGCCAAAGTCCTGAAGGATTCACTTCACGAGAAGTTCCCTGACGCTGGTGAGGACGAGCTGCTGAAGATTATCGGTAACCTGCT
TTACTACCGATACATGAACCCAGCCATCGTCGCTCCCGATGCCTTCGACATCATTCACCTGTAAGCAGGGGGCAAGCTCACCAAAGACCAGCGC
AGAAACCTGGGCTCCATTGCCAAGATGCTCCAGCACGCGGCGTCCAACAAGATGTTTCTGGGCGATAATGCCCACTTAAGCATCATTAATGAGT
ATCTCTCGCAGTCCTACCAGAAATTCAGACGGTTTTTCCAATTGGCTTGTGACGTCCCAGAGCTGCAGGATAAATTTAACGTGGATGAGTACTC
TGACCTAGTCACCCTCACTAAGCCAGTTATCTACATCTCCATTGGCGAAATCATCAACACCCACACTCTCCTGTTGGACCATCAGGATGCCATT
GCTCCAGAGCATAACGACCCCATCCACGAACTTCTGGACGACCTTGGGGAGGTGCCCACCATTGAGTCCCTTATAGGAGAAAGCTGTGGCAATT
CAAACGACCCCAACAAGGAGGCTCTGGCTAAGACGGAAGTGTCTCTCACGTTGACCAACAAGTTTGACGTGCCTGGTGACGAGAACGCAGAGAT
GGACGCTCGGACCATCTTACTGAATACAAAACGTTTAATTGTGGATGTCATCCGGTTCCAGCCAGGAGAGACCTTGACTGAAATTCTAGAAACC
CCAGCCACCAATGAACAGGAAGCTGAACATCAGAGGGCCATGCAGAGACGGGCTATCCGCGATGCCAAAACCCCTGACAAGATGAAAAAATCAA
AGCCCATGAAGGAGGATAACAACCTCAGCCTCCAGGAGAAGAAAGAGAAGATCCAGACTCGCCTAAGAAGCTAACGGAGCTTGAAGACGGTGGA
CCCAAAGAACAGATACCAGGAACTCATCAACGACATTGCCAAGGATATCCGGAATCAGCGGAGATACAGGCAGAGGAGGAAAGCTGAATTGGTA
AAACTGCAGCAGACGTACTCGGCGCTGAACTCTAAGGCCACCTTTTACGGCGAGCAGGTGGACTACTACAAGAGCTACATCAAAACCTGCTTGG
ATAACTTGGCCAGCAAGGGCAAGGTCTCCAAAAAGCCTAGGGAAATGAAAGGCAAGAAAAGCAAAAAGATTTCTCTGAAGTACACAGCAGCGAG
GCTGCATGAGAAGGGCGTCCTTCTGGAGATTGAAGACCTTCAGGCAAACCAATTTAAAAATGTTATCTTCGAAATTGGTCCAACAGAAGAAGTT
GGAGACTTTGAAGTAAAAGCCAAGTTCATGGCAGTTCAGATGGAGACTTTCATGTTGCATTATCAGGACTTGCTGCAGCTACAGTATGAAGGAG
TTGCAGTTATGAAATTATTTGATAGAGCTAAAGTGAATGTCAACCTCCTGATCTTCCTTCTCAACAAAAAGTTCTATGGGAAGTAA
HUMAN SEQUENCE—GENOMIC
CCAAGGGAAGGATGCGGAATGTCTGTTTTCTACTAAGGGCTTCTGCTGGGCCTTTCCAATGTCCAGTTTGAGTTGGTCTGGGACCTGGCCTCCG
CAAACACCCTGCAGGCTCCGGCCGCCGGGGATGAGGTGGAAGACTGGGCAGAAGAGAGGCCGCAAAGGCCCGAGGGCTGAGCTGCTCTGCGCTG
GGGAGGGGCGAAGTGCCAGAGCGCGGGAGACCTCATGGTGGGCACCAGGCTGCTCCGCGGTAGGTGGGTGAGGCCAGGAGATTCACATCTGCAG
ATGCCACAGTGAGGATACAGCATTTAGATCCCTCGGCTCCAAACAGGCGGTTCCGGGGGACCGGTAGCTGGGGAATTGGGTTTAGCACTTTCCA
ACGCTTGGAACTGGACATCCACGGCTCCGAGTCCTTTGAGGTCTTGCTCGGGACTACGTTTCCCAGAAGGCTAGGCGATAATTGACAGCTCTAT
CAGCCTATGGACAGAGAAAAATTCTAGCCATGGAAACTGAAAAGCCAATAGCAAGAGGATGGGGGCGGTACTTTCCGGCCGCTGGCTGTCGAAG
CCGGAGTCCCACCTGTGTCCCCACAGCCCTGTCACGAATCCCGGTCGGGTTCTGGGAGGCACAGCCTCGGGGTTGCGGGCCGGGTGCGGCTCGG
CGGTGGAGGACTCACTTCCTGCTCCATCCCCGGCTGGGCCCTGGGGCGGTGAGTGATTCAAGGAGAAAGACCGCGGCGGCAGCGGCGCGCTGCT
GAGGGGCGTGGAGGGGGCTTGCCCGCCACCCAGACGTTTTCTCAGCGGGGTTCCCGCGCTGGCCTTTGGGAGCCCCCGGGTCTTGCTGGGCTGT
GGGAGAGGTAGCTGTGGTCTCTATACCGTCGGCGGGATAAGTCATTCTCTTAATCAGTTTTCTTGCTTCTCGATGGTGGTCCCAGGGTTTCAGC
TGATCTGTGACCCTCTGACCGCCGCACCCCGGTTAGCGCGCGAGCACCGAGAAGAAGAACGGGGTTCGGCCCCACGCGTGCAGTCATGTTCCTA
TTAATAAACCCGGTGAACGCACTGGAGCTTCTCACAGTGGCACTTAGTCACAGCCCCTCAGCGCTGTGGGGCCTTTGAGGTCACCTGTGCATGG
GGAAAGGGACGGGCTGGGAAACAGGGTTTCTATAACCGTGGCTAAGACGTCTCCCCTCCTCAGTGGCGCTGTCTCCAGCAAGTGGGCCCTGCAG
CTGTGTTTTGTAATGAATGTAGAAATAAAAATTATACTCAAAACTCTTTTTTTTTTTTAGACGGAATTTCGCTCTTGTTGCCCAGGCTGGAATG
CAATGGCGCTATCTCCTTTCACTGCAGTCTCCACCTCCCGGGTTCAGGCGATTCTCCTGCCTCAGCCTCCCAAGTAGTTGGGATTACAGGCGTG
TGCCACCACACCCGGCTAATTTTGTATTCTTAGTAGAGACAGGGTTTCACCATGTTGGCCAGGCTGATCTCGAACTCCTGACCCCAAATGATCC
GCCCGTCTCGTCTTCCCAAAGTGCTGGGATTACAGTCATGAGCCACAGCACCTGGCCAATACTGAAAACTCTTATTACAATTTTTACTTTCTAA
TTACATTATTGTTGTTTGTTAAAGTTTTAAACCAGAGAGATGGTTTTTAGATCCTTAAAAACTCAATACTTTATAAATGACCAATAATAATGTC
TGTAAATACAAAAATATTTCCCCCAAAAGTGTGTGTGTGTGTGTGTGTGTGTGTGTGTGTGTGTGTGTGTTCCAGTAGTAAATAGGTGATTTGT
AGTGGGACAAAAGATGTGAAAGAGTAAAACTTCTTCACCACAGTCTGGTCCTTCCTCCCTTGAAATGCAATATTCTTTGTAGTGGGTGGTCATG
CTGGCTGCATTTTTTTTTTTTTTAAGGGAGAGACAGGGGCTCACTCTGTTGCTAGGCTGGGGTGCAGTGGCGCCATAATAGCTAACTGCAGCCT
GGAACTCCTGGGCTGAAGTGATCCTCCCACTTCAGCCTCCTGAGTAGCTGGGACCACAAGTGCATGCCACCATGCCTGGCTGATTTTTAAAAGT
TCTTTGTAGAGAAAGAGGTCTCAATATGTTGCTGAGGCTGGTTTATTGGAAACTTTTTAAGGGAAAAAACATTATCTTAATTTCTATGTGACTC
CTATGCCTGGCAACATATTAATTGTTGAATAAATGACTGATCCATCCAGTAATTCACTCTTGACTATCCTCCAAAAAGTAATGTTTTTCATGAT
ATTGCTGTCGATTTATTAATATATTAATGATTGGTTTCAGCCCCTAAGAGTTGTGTTTTGTGCTTATTCCCACTTTCACTTATTTTTACAACCT
ATCCATGTCTTCTTTTTTTTAAAAAAAAAATGTCTTTTTCAGTCTTATTTCTGACTCCATTAAGATCTTGAAATAGAATGGTATCCAACACAGT
TCCCTTTGGGGTTCTGTATAATCTGTCCCTTGAGGTTGACACCAAATCTGCAAGCCAAGAAGTTAGCTGCTCCCATAACTAATATGAGTATAGT
TAAGGCCACAATTTCCTTGCTTGCTGAGGAAAATGAAGTTGGGGAGAATCAAAAGCTTTCCAGAAATCAAATTACACCTGCCAACTCTCTCCTT
TGTTTACCTTGTTCATCGTGGACAATTTGTTCCTCAAAAAGTCAATGTGTCTAAAAGACTGGGTTCTGTCGATGTGTTTGGGAGGCCTAGGCCT
CTCTTAGTAATGGGAAAGTTGGCCAGGAGAAGACGTATGTAAAAACAGACTTAGAAAAGCACCCCTCTGTGGTTGACAGAATTTACCTCAGGAC
CATTCTGGTCAGGTGTGGGGGGTTGTTTGTGTACACTGGTGGCCATTTTTTGGCTTTCAGACAATGGTGAGAATATCTAGGGAGCTACTGATGC
ACATTGAGGTCTCTTGCTTTAGGAACTGATGACGCTTGATAATGTGGCTGGGGACTTCAGGGAAGAGTGGGTTACCTGGACACTGCTTAGAGAA
TCCTCTCCAGGAATGCCACCCAGCACAGTTAGAAGAATGGGCTCTCAAAGGGTAAGAATGCTACTCTCCTTTATTTCATTTATTTATCTTGTTT
TTTTTGAGACGGGGTCTCGCTCTGTCATCCAGGCTGGAGTGCAGTGGCGCGATCTCGATTCACTGCAATCTGTGCCTCCTGGGCTCAAGCGATT
CTCCCACCTCAACCTCCTGAGTAGCTGGGATCACAGGCATGTGCCACCATGCCTAGCTAATTTTTTTTTTTTTTAGTAGAGACAGGATTTTGCT
ATGTTGGCAAGGCTGGTCTCACTATGCTGTCCAGGCTGGTCTCAAACTCCTGAGCTCAAGCAATCCACTGGCCTCCGCCTTCCAAAGTGCTAGG
ATTATGGGCGCCACCATGCCTGACCACTGCTCTCCTTTAATTTGAGACTTAGATATTTGGAGAGGGCCCAGGTCTGAGGAGTTTCTGGTATGTT
GGCATCTTAGAGCTCCAGACCTCTGTAAGATTAATTGTTGCCTCCTTGGGAGAAAAACAATTACTTCCTTATGCATTTTATGGTTTGCAAATGT
CTTTGCTATTTATTGTCCTTTTAATATCTCATCATAACTTTTTGACTTATGTATTACTATCCCCCTTTTCCAGCCTGGGTTGGACTCTCACCTC
TGCCACTTAACTTCTGAGACTTCTGAGGTCTTTGTGGAAAAGGAGGTAATTTTTTCTGTCACTTAAAAACAGGCTGGGGGCAGTGGCTCACGCC
TGTAATCCCAGCACTTTGGTAGGCTGAGGTGGGCGGATCATAAGGTCAGGAGTTCGAGACCAGCCTGGCCAACATGATGAAACCCTGTCTCTAC
TAAAAATAAAAAAATTAGCTGGGTGTGATAGCGGGCGCCTGTAATCCCAGCTACTCTGGAGGTTGAGGCAGGAGAATTGCTTGAACCTGGGAGG
TGGAGGTTGCAGTGAGCTGAGACCACGCCATTGCACTCCAGCCTGGGCAATAGAGCAAGACTCTGTCTCAAAAAAAAAAAAAAAAAAAAAAACA
AGGCTGGGCGCGGTGGCTCATGCCTGTAATCCCAGCACTTTGGGAGGCCAAGGCAGGGAGATCACAAGGTCAGGAGATCGAGACCATCCTGGCT
AACATGGTGAAACCCCGTCTTACTAAAAATACAAAAAATTAGCCAGGCATGGTGGTGGGCACCTGTAGTCCCAGCTACTCGGGAGGCTGAGGCA
GGAGAACAGCGTGAACTGGGAGGCGGAGCTTGCAGTGAGCCAAGATCATGCCACTGCACTCCATCCTGGGCGACAGAGCAAGACTCCATCTCAA
AAAAAAAAAAATAAATAAAAAAAACCAAAAACACCTCCAATGCTCTTTCTCAAATATCATGGCTACCCTTCCTCCTGCTTTATATTTTTAGTAG
AGATGGGTTATCTCCATGTGGTCAGGCTGGTCTCAAACTACTGACCTCAAGTGATCCGCTCACTTCGGCCTCCCAAGTGCTGGGATTATAGGAG
TGGGCCCAGCTCTAATTTTTGTATTTTTAGTGGAGTCGAGGTTTCACCATGTTGGCCAGGCTGGTCTTGAACTCCTGACCTCAAGTGATCCGCT
GGCCTTGGCCTCCCAAAGTGCTGAGATTAGAGGTGTGAGCCACTGCGCCTGGCCTACTTCATCTATTATACAAGTAATGTATACTCATTCAATA
AAAAGTCAAGCAAAATGAAAACAAACATCATCCCTAATCCCACCAATGAAGAGTTACTATCCTACCAGCATTTTTCTAAGCATATATACAATTT
TAAAAGAAGCACAAAAGAATAATATTGGGATTATGCTGTATATTCAGCTGTACATTCTTTCTTCCATGTTGTATTGTGAGTATATCTTTCTTTA
CATCACTTAAAATAACCACCTAGTATTTTGTTTTATGGAATACAATATTATTTTGTTGATGAAGTCAAACATTAGATTGAACCATATATCTGAC
CTTTTTTTAACCTATAAAAATAGCAATTGCCTGAGCTACTTGGGAGGCTGAGGCAGGAGGATCCCTTCAGCACAGGAGTTCGAGGCTGCAGTGA
GCTATATAATTATTCCTTTGTTGTGGGACATTTAGGTCATTTCTAAACTTTCACCAGGTTGTGATAAACATCTTTTAGCTCGTTTTTTAAAAAG
TATTTTTTATTGAAGTATAATTTTATCATTTTCTTAGAATTCTCAGAATTAGAATTGGTGGGTCAAAGAACAGATACATTTCTAAGGTATTTAA
TACTTTGCAAAATGCCTTCCAAGAAGATTGAATCAATTGATTTATTCCCACAAACATTATATGAAAGTGCGCATTTCTTTATATTCTACCTTTC
ACAGTGGGTATTATAATTTAAAGAGTCTGGGCCTGGGTGTGGCAGCTCATGTCTATAATCCCAGCATTTTGGGAGGCTGAGGCAGGAGGACCAC
TTGAGCTCTGGAGTTCCAGACCAGCCTGGGCAACATAATGAGACCCTGTCTCTACAAAAAGTTACATTAACCAGGTGTAGGGGCTTGTGCCTCT
GGTCCCAGCTACTTGGGAGGCTGAGGTGAGAGAATCGCTTGAGCCAGGAGCTCGAGGATGCAGTTAACTGTGATTGTACCAGTGCTCTCCAGCC
TGGGTGACAGAGCAAGAGCCTGTCTCAAAAACAAACAAACACACACACAAAACACAAAAAAAGAGTCTGGGAAAAATAGCAATAATAGTTAACA
TTTATTGAAGTTTTTTTTGGAGATGGAGTCTCAATGTGTTGCCCAGGCTGGAATGCAGTGGTGCAATCTCAGCTCACTGCAACGTCCGCCTCCC
AGGCTCAAGCAATTCCCATGCCTCAGCCTCCCAAGTAGCTGGGATTACAGGTGTGCACCACCACACGTGGCTAATTTTTGTATTTTTAGTAGAG
ACAGGATTTCTCCATGTTGGACAGGCTGCTCTTAAACTCCTGGCCTCACAAGTGATCCAACTGCCTTGGTCTCCCAAAGTGCTGGGATTACAGG
TGTGAGCTACTGCACCTGGCCAATGGATCTTATTCTTATCCCAATTTTTTTTTTTTTTTTTGAGATGGAGTCTCTCTCTGTCGTCCAGGCTGGA
GTGCAGTGGTGTGACCTCGATTCACCATAACCTCTGCCTCCTGGGTTCAAGCGATTCTCCTGCCTCAGCCTCCCGAGTAACTGGGACTACAGGC
CTGTGCCACCATGCCCGGCTAATTTTTAGTAGAGACGGGGTTTCACTATGTTGGCCAGGCTGGTCTCAAACTCCTGACCTCGTAATCCGCCTGC
CTCGGCCTCCCAAAGTTCTGGGATTATAGGTGTGAGCCACTGAGCCTGGCCTCTTATCCCTATTTTATTGATGAAGACATTGAGACACAGAGTT
TAAGTAACTTTCTTAAGGCCACACAGCTTGTAAGAGGCAGGATGATGACTCAGACCCAGGCAGTGTGACTCTTGAGTTTGCACTCATTACCTCT
ACACTATATTGCCTCAGTGTATCATTGTTATAATGGAATTTCTTTGATTATAGTGAGGTTAAACATTTTTCGTATATTTACAGGCTACTTAACA
ACTGTTTGTCTTTTTCTCATGGCTTTTTTGACTGCTGCCATTCTGGGAAAATTTTAAAGCCCAACATTATAGTTAACTTCCTTCATTGTTTCTA
AGGGTGAGGTCATGGAGGCTTGGTTTCTGATAAAATCCTCTCCATATTTAGCTTTAGCCTTGTACCTTCAGAAATGGTTATTGTATTTACTTCA
GAGTTTCTGTCACCTGAGTCTGGTTTTTTTTCTTTATGGGAGGAAAAGGGCTTTTTTTTCTTAACATTTTGGGGAAATCACCGTGTTTTATCAC
TGTACCATGGCTGCACTTTTTTAATGACACCAAATACACGTTAGAGCTCCCCAGGTGTTCTTCCATACCTAGGCAGGGAGTAGGGCTTGGATTT
TGCTCAGGCCTTGTGTGATTGGCTCAGACTCACATCCTATGTTTACATTCCTGTGTAGAATTTCCAGGCCTAGTGTAATCTCCCAGCCGGAGCA
GAAAGAAGAGCCATGGGTCCTACCACTCCAAAACTTTGAGGCGAGGAAGATCCCGAGGGAAAGCCACACAGGTGAGATGTGAGTGCTCCCCAGT
GGAAGGAAATCTAGCATTTCAGCCTTGTTTAGATGAAGAATTTGGAATGCTGCCTTAGAATTTTGTTCTTACTAGCAGACATTTTTTTTTTTTT
TTTTTTTTTGAGACGGAGTCTCGCTCTGTCGCCCAGGCCGGACTGCGGACTGCAGTGGCGCAATCTCGGCTCACTGCAAGCTCCGCTTCCCGGG
TTCACGCCATTCTCCTGCCTCAGCCTCCCGAGTAGCTGGGACTACAGGCGCCCGCCACCGCGCCCGGCTAATTTTTTGTATTTTTAGTAGAGAC
GGGGTTTCACCTTGTTAGCCAGGATGGTCTCGATCTCCTGACCTCATGATCCACCCGCCTCGGCCTCCCAAAGTGCTGGGATTACAGGCGTGAG
CCACCGCGCCCGGCCACTAGCAGACATTTAAATTTGACTTAATGGTCAGTAGTTGACTAAATAAAGTCTTCATTTTGATTAAGCATTTCACTTT
TGTCTAACCTATGTTTTTTTTTGAGATGGAGTCTCCCTCTGTCTCCCAGCCTGGAGTGCAGTGACACAATCTCTGCTCACTGCAACCTCCGCCT
CCCGGGTTCAAGTGATTGTCCTGCCTCAGCCTCCCAAGTAGCTGGGACTATAGGCACCCGCCACCACGCCCAGCTAATTTTCATATTTTTAGTA
GGGATGGAGTTTCACCACGTTGGCCAGGTTGGTCTCGAACTCCCAACCTCAAGTGATCTGCCTGCCTCGGCCTCCCGAAGTGCTGGGATTATAG
GCGTGAGCCACCACGCCTGGCCTAAACCTATAATATCTTCTAAAGAAACCGCACAATATTGACAATATGTGGTCTCCGCTTGAAGGATATCAAG
ATTCATCTGGCCATCTTAAAATTTAAAAAGTTTAGGAATTCACGTGTTCTGACAGCTCAATGAACCTCAATAAACTTTTCACAAAGTGAAAACA
GAGCGGTTGAATTAAGTGCAGTCTGCTGCAGAAACAGGGATTTTAATCTTTGTCACTGCTTTCTGATATTCCTTCCTGATAACTGATAACTTTA
TTCTCTGTTACTTACATTAATTTTTCAGACTGTGAGCATCAGGTGGCAAAGCTCAATCAGGACAATTCTGAAACAGCAGAACAATGTGGAACAT
CCTCAGAAAGGACCAATAAAGATCTTTCTCATACTCTTAGTTGGGGAGGAAACTGGGAGCAAGGCCTAGAATTAGAAGGGCAACATGGAACCCT
TCCAGGAGAGGGCCAGCTGGAGTCCTTTTCACAGGAGAGGGATTTAAACAAGCTCCTGGATGGATATGTAGGAGAGAAGCCTATGTGTGCAGAA
TGCGGGAAAAGCTTTAACCAGAGTTCCTATCTCATAAGACACCTAAGAACCCACACTGGCGAGAGGCCCTATACGTGCATTGAGTGTGGGAAAG
GCTTCAAACAGAGCTCAGACCTTGTCACCCATCGCAGAACACACACAGGAGAGAAGCCCTACCAATGCAAGGGGTGTGAGAAGAAATTCAGCGA
CAGCTCAACACTCATCAAACATCAGAGAACCCACACAGGGGAGAGACCCTATGAGTGCCCAGAGTGTGGAAAGACTTTTGGGCGGAAGCCACAC
CTCATAATGCACCAAAGAACCCACACAGGCGAGAAGCCCTACGCGTGCCTGGAATGTCACAAAAGCTTCAGTCGAAGCTCAAATTTCATCACTC
ACCAGAGGACCCACACAGGGGTGAAGCCTTACAGGTGTAATGACTGTGGGGAGAGTTTTAGCCAGAGCTCGGATTTGATTAAGCACCAACGAAC
CCACACGGGAGAACGGCCCTTCAAATGCCCGGAGTGCGGGAAGGGCTTCAGAGATAGTTCTCATTTTGTAGCTCACATGAGCACTCATTCAGGA
GAGAGGCCTTTCAGTTGTCCTGACTGCCACAAAAGCTTCAGTCAGAGCTCACATTTGGTCACGCACCAAAGAACACACACAGGTGAGAGACCTT
TTAAGTGCGAAAACTGTGGGAAAGGATTCGCCGACAGCTCCGCCCTCATTAAGCACCAACGAATCCACACCGGAGAAAGACCCTACAAATGTGG
AGAGTGTGGGAAGAGCTTCAATCAGAGCTCCCACTTTATTACCCATCAGCGAATCCACTTAGGAGACAGGCCCTATCGATGTCCTGAGTGTGGC
AAGACCTTCAATCAGCGTTCCCATTTCCTCACACACCAGAGAACGCATACAGGAGAAAAACCTTTCCACTGTAGTAAATGTAACAAGAGCTTCC
GTCAGAAAGCGCATCTTTTATGCCATCAAAACACCCATTTGATTTAGGAAGTAGTCTTTGGTGTTCAGCTGCTCCCTTGCACATTTTCATTGCT
ACTGTCTTCAAGCACCCCAAATAGAGAAAACCTGGGCGTCAGTGGCTCAATTTGGGCCCTGATCTATTCTCCCTCTTTCTTGTCTATGTTATAA
CAGAGAGGATAAACTTAAAGGGTCCAAATAACGGTCCGAATACAAAAGGCATTCCTTCAGTGTGTGACTGACTCTTAGGGAAATGTGAGTTTAA
TAGTTGATGCCCGCCAGGCGTGGTGGCTCACCCCTGTAATCCCAGCACTTTTGGGAGGCCAAGGTGGGTGGATCACTTGAGGTCAGGAGTTGAG
ACCAGCCTGGTGAGCATGGTGAAACCTCATCTCTACTAAAAATGCAAAAATTAGCTGGGCATGGTGGCACGTGCCTGTAGTCCCAGCTACTCGG
GAGGCCGAGGCAGAAGAATCATTTGAACTCAGAAGGTGCAGGTTGCAGTGAGTTGAGATCATGCCACTGCACTCCAGCCTGGGCAACAGAGAGA
GACTCTGTCTCCAAAAAAATTAAAAAGTTGATGCCTAGTTACTAAATGAGAAGTGAGAAAAATGTGGCCTAGAGATCACATGTTCACCACCTAG
TACAGTGCCTGGCACAACATAGATGCTCAATAACTAAATGGTCCCATCATTATTAATGATTAATAGTTGAGGTCTTATATAAAGGCTTTAATGC
AGTACCTGGCCCTTAAAAGACACTCAGTACAAGATTGGTGGCTTTTATCAGTCTTATTACTCAATTAGAAATTTATTAGTGTAGTCCCCCCGCC
CCAAACTCAGAACGTGAAAGAAATTAGATTGGAACTAGAAGGTTTGGGGTTGGAATACACAAATTGAATAAAATGGAAATGCACTGCCAGTTCA
GTGCTGTGCAGGCATTAGTCACCAGAGGTCTCACTGCCATGACAAGGCCAAATTATCGTAGAGGATGTTTGCGGTCTTGTGACTTGGAGGCTGA
AAGAATTTCAGAAGCTCTTTTAAATGGCAGTGTATGGCAGTGTATCTACCAGAGGTTTGCTGTCATCTGACACAGAGAAAATATCCTACAATGA
ACAAGCCAGAGGGACCTGGTAGAGGACTATAAAATTGTGGAAGCAAAATTGCTGAGAATGTCAAATGATATTACAGGGATCCTCCCTGGCATTT
AGCTGAAGGAAGCAACTCTTGTTTTCTAATTTGCTGGGTCATTGGCCATTTAGTTTTAGGTTAATATAATTCTCTGATCCTTTTAGGGCCATCC
AGGTTATGCACTAGTACATTCCTACATTCAATTGAAATAAATTGAGGGACGGCAAGTGTGTTGGAAAGAACACCGACTTCATTGAGAAGGTAAA
GTATTTGAGTCCTGGCCCTGACGCTTAATTTGGCCAGACTTTCATCTTCTCCCAGCCTCAAGTTTTACCTACCTCACAAGGTTGTTGTGAGGAT
CTAAAAATACACACACACACACACACACACACACACACACACACACTTTGTTGGTTAACTATAAATGTAATATCTCTATGTTATAATTCTGTTG
CTAATGTCTTTTTTCCAAGAAAATTTTGGCTAATATTTCTTTAGGTATTCCTTTTTCTCTCATAGTGAGGGATTAAAAAAAAAAAAAACTGTTG
AAAAATTAGGGCGTAAAAATGCTAAATGACATGACTCATCATGGGCCACGTAGTTAACAGAAGAGCCAGATTTGGCTGCAAGTCACTAGATTTC
CAGCCTGCAGTCCTCCTCTGCAACAACAGACCAGCTCTGGGATTTGTTACAGTGCCTGTGAGACATTACAGGACTGGAGGACCCATATTATATC
CATTAAACCAGTCTGAATTTGGAAATGATGGAGGGTGTAGTCTAAGTTGTAGGGAGCTTTGCAAGAACCTGTGCTGGGGTCCTTGATCCTGGTG
GAATGGGGGTGGAGGACTGACCGCAAATGCAAGGGGTTAAGGAGGGAGCTGGGTAGTTATCACTTTTTAGAGTGCAAAGTGGTGGATTAAAGGT
TTCTTTTCTTTCATTCTCTTCCATTATATGGAATGCCATCTGAGTGCTGTGGCTCATGAAGGATAGAACTCAGCTGATAACCTTACCTCAGTTT
TTGAAAGCATCATTAGATAATTGACCAGAAAATTTTTTTTAGTTAATCCAGTGCAGTGGTTCTCAAACTGTGAGCCCAGTCCAGTAGCATCAGC
ATTATCTGGGAACTTTTTAGAAATGCAGATTCAGGCCGGGTGCAGTGGCTCACACCTGTAATCCCAGCACTCTAGGAGGCCGAGGCGGGTGGAC
CACCTGAGGTCAGGAGTTTGAGACCAGCCTGGCCAACATGGCAAAACCCCACCTATACTAAAAATACAAAAATTATCCCAGGTGTGGTGGCATG
TGCCTGTAATCCCACCAATTTGGGAGGCTGAGGCAGGAGAATCACTTGAACCCAGGAAAGGAGGCTGCAGTGAGCTGAGATCACACATGCTGGA
GTGCAATGGCATGATCTTGGCTCACTGCAACCTCTGCCTCTCAGGTTCAAGCGATTCTCCTGCCTCAGCTCCCGGAGTAGCTGGGATTACAGAT
GCATGTCACCATGCCCAGCTAATTTTATATGTTAAGTAGAGACGGGGTTTTCACCATGTTGGGCCAGGCGGGTCTTGACCTCCTGACCTCAGGT
GATCCACCTGCCTTGGCCTCCCAAAGTGCTGGGATTACAGGCGAGAGCCACCTCACCTGGCCACCTATCACTTTGATTTTCATGTTGTTTGGCT
ATGGTAAAATGTGAGCTCTCGAAGGGCAATGTGAGATTTGCTTTGTGGCCCTGCCAATCCCCTCCCTCCTCCCTGTCTTCCTGCCCACCCCCCC
CCACTCCCCCGCCAGCCATGAGCAGGGAATATTTCAATGCTATTGCTGAGAGTGGAGGTAACCCTTTCTATAGTTTTCTTTTGTTTCTACCTCA
TGACTAGATGATTCACTGCTTGAACATGCTCCACACATCCGTTCTTTCTCACTCCAAAATTGAGGTCATCACCAAGCCCCATTAATTTTACCTC
CCAGATTCCTCTCGAACCTATCTGTCAAATCTGTCCATCTTCACTGCCACCCTTCAGTACCAAATGACCAGTCTCTTACCTGAATTCCTGTAGC
AGCCTCCAAACTGATCTTCCTGATATGATTTTTGCTCTGAAAAAACTGGTTTCACTCACAGAAACCAGAGTGAACTTTTAAAATCCTAAATCGA
ATCACATCACTTCTCAGCAGCTTTCCATTGCTTTTAGAATGAAGACCCAAATCCTTACCCAGGCCTAAGAGGCCCTTGTGGTTTTGTCCCCTCC
CCCTCCATCCTCTTGTGATATCCCCTTCCCTCTCCCTTGCCTCACCTCAGCACTCTTGAGTTCTCTGCTCCTTGGTCATGCCAAGGTTGTGTGC
TCTTTAGACCCTTGGTACTAACTGTTCCCTCTGCCCAGAATGTTCCTCGCCCAGTCCTTTGTGTTGCCTCCTATTTGTCAACCAGGTTTCAGCC
TAAACCTATCTCCTTAGGAAGACTTTCCCTAACTATCCCATCTAAATTAGTCACCCTCCATCACATTATCCTCTTTTTTCATCAAAGTCCTTAC
ACCTGTCTGGCAATTTCTTATTAATTGATTTGTTTTTGGTAAACTCCACGAAAGGTGGGAGTCATTTCTCTTGTTTCCATTCTTTCCACAGCAC
TTAGAACAGTGCCTGGCACATGGTAGGTGCTCAATATGTGTTTACTGGATGAATTAATGAGTGCATGCTCATGGGCAAGGATCTCATTGTGCTG
GAAAAGCAGCAGAGTGAGACAAAACTGGACAGGTGAGTGAAGGTCACACCAAGAAGGTTCTTAGCTGCCAAACTAAAGGGTTTGGATCTTACTC
TCTGGAGAAAAGGGTGTAGCTAGTGGTTTTATAGGAGAGGAGAGGCGCGATCCGCACTTGAGAAGGTCAATTTGGCCGCTGTGTGTAGGTCATA
TTACGGAGAAAAAAACTAAAGGCTTTGAGACAATCCATGATGTAAAGGGCTTAGCACAGTGCCCAGCACGCAGTAGGTCTCCAGCGAGTCGTTA
TCACCAAGCACCAGGGCAGGCACCACAACAAAAAGATAAGATCCCTAACCCTTCTTTTACCTCGAAATAATTCCTCCTAGACCCTATCATCGCA
TTCCTTCTCTTCCCCTTTTGTCAAACTTCCACTCACATGTAGATATTCTCAGGGTTATCATGCCTAGGCCTTTTTAATAGGGCCACTTACCCTA
GCATGCTTCTACAACTGGACCCTCAAAGGTCTTTTAGGGCTGAGTGTGGTGGCTCACACCTGTAATGCCAGCATTTTGGGAGGCCGAAGCCAGA
GGACTGCTTGAGCCCAGAGGTTCAAGACCAGCCTGGGCACCATAGCCAGACCCTGTCTCTACAAAAAATAGTTTCAACAAATTAGCCAGGCATG
GTGATGTGCGCCTGTCATCCCAAGTACTTGGGAGGCTGAGGTGGGAGGATTGCGGATTGCCTGAGCCCAGGAGTTCAAGGCTGCAGTGAGCTGT
GATCATACCACCGTACTCCAGATTGCGTGACAGAGCTAGAACTTGTCTCTTAAAAAAAAAAAAAGACTTAATTTCCACATCTGAAGGCCTCTTG
TTAGTCTTCTTCTGATTCAACAACTCTTTACTGTTTGATGCCATTTACATATGTTTATTATTTTTTAGAGATGGGGTCTCATTCTTTTGCTGAG
GCTGGAGAGCAGTGGTGCGATCATGGCTCACTGCAGCCTCAACCTCCCAGACTCAAGCAATCCTCCTGTCTCAGCCTCCTGAGTAACAGCTAGG
ACTACAGGCACATGCCACCAAACCCGGCCTTTTAAAATTTTTGGTAGAGGCCAGATGTGGTGGCTCATGCTTGTAATCCCAGCACTTTGGGAGG
TCCAGGCGAGTGGATCACCTGAGGTCAGGAGTTTGAGACCAGCATGGCTAATATGGTGAAACCCCGTCTCTAGTAAAAGTACAAAAATTAGCCA
GGCGTGGTCGTGGGCGCCTGTGATCCCAGCTACTCGGGAGGCTGAGGCAGCGAGAATCACTTGAACCTGGGAGGCGGAGCGTTGCAGTGAGCTG
AGATCGTGTCATTGCACTCCAGCCCAGCTGACAAGAGCGAAACTCCATCGCGGGGGATGATAATAATAATAATAATAATAACTTTGCTAGAGAC
AGGGTTCCCCTATGTTGCCCAGGCTGGTCTTGAACTCCTGACCTCAAGCAACTCTCCCACCTTGGCCTCCCAAAGCGCTGGGATTACAGGTGTG
AGCTACTGCACCAGGCCCATATGCCTTTTTAAAAAAATTATCTTTTCCATTGGTGACTATGAGGTTGAGAGATGATTCTCCTACATTTCTGGCT
GCTCCTCTTCAAGTACCTTCCCTGGCTCCTCTGGATTTTTTTGTTTTGTTTTGTTTTGTTTTGTTTTGTTTTTGAGACAAAGTCTTGCTTTGTT
GCCCAGGCTGGAGTGCAGTGGCAGGATCTTGGCTCACCAGCTCACTGCAACCTCCACCTCCCAGGTTCAAGGGATTCTGGTGCCTCAGCCTCCA
GAGTAGCTGGGACTACAGGCCCAGCTAATTTTTGTACTTTTGGTAGAGATGGGGGTTTCACCAGGCTGGTCTTGAACTCCTGCCTCAGGTGATC
TGCCCACCTCGGCCTCCCAAAGTGCTGGGATTCTAGGCATGAGCCACCGCGCCTGGCCTGGCTCCTCTTCTTCTTCCACTCAGATATGCCTGAC
CCTGTCAACACTTTGGTTGAGGTCTTCTTTCTTCTTTCTTTTTTGCTCCGCACATTTAGCTTATGACTTCAACCATCATTTCTCAGAGCATGGG
TCTGGCTCAACCTCTCTCCTGAATTTCAGACCTACAAGTCTAGCTACTTGGTGGAGACCTCCCCAGAATGACCTGCTGCTTCCCAAAAGCAGAC
TCTCCAAATTACAGTCAGTATCTCCCCCGGAAGCATTCCCCCAGGCATTTCTCTTTCTGCCTTCAATTCCCCATTCTCCTACATTGCCTTGCCA
GAAGCCTGCTGGTCAGCTTGGATTTCTTTTTGTCCTTTTTTTTCTATATTTTGCTGGTGCCTAGTCATGTAGTTGCTGCCTCTACACTTTCTCT
TCTTTAAAAAAAATTATTAAAGCACCACGTGCTTGTTGTAAACATTTCCAGAAAATACAGAAGTGCTCAAAGTGAAAAAATGGAAATGCCTTGT
CCCTTCCTCATTCCCTGCCCTAACCTCACGCCCCAGATTCAGCTATGTAATAGTCTGTCATGCCAAGTCTTATTTCCAGCTCCTCTTTTCCATC
CCCACTGCCATCATCTGAACTAAACGGATTGTTTTCCATCTGGTCTCCTTGGCTTTTCCTTTCAGTGCAGCTCAACAGACATTAATCAAGTGCC
TTCCACACACCAAAGTCCTACCCTAGATCCTAGAGGTTCAGAGACAAGTAAGATAGTTAAAGAGATCCACATTCCAGAGCTGTTTAACTTTGGG
CAAGTTACTTAATCTCTCTGACCCTTACTTCCTTATCTGTAAAATGATGCTAATCCCAGCACCTTTTTCATGGGTTTGGACGAGCATTAATGAG
ATGATCCATGTAAAACTCTTTGTACTAACTACCTGGTACACTGTATCTCCTCCATAAATGTCAGTGACAACAATGATAATAATGACAATGTTTG
GAGGAGTTTATAGCTTAATGGAGAGACTTAAAGCATAAGAATTATCTAGGCGAAGAATGATGAGAAAATATTTTTGGAAAAGGAAAACAAACAG
TTCTACTAAAATTAAAAGGCTGATGTAGAGACTTGGGAAACTGGGAGGTAAGAGCTCCGACTCTGTCCTCTAAGACAGTAATTCCCGAAGTGTG
AGCAAAAGTCCACCTGCATCAGTCTTACTTGGGGTGATTGCTCAAAATGAGGATTTAATGGCTGCACCTCCGAGCAAGTTGGTAATTTACATAT
CGGAATGCTATCCATCAAGGAAAATGGGCAGACTACAGTTACATGCATCAACACAGACAAGCTTCAAACAATATTGAGTGTAAAAAGCAAGACA
TAGAAATATATATTTAGTAAGAGTAAAAATACAGTAAAGGTAAAAAAGAGGCAAAACTAAACAATATATTGCTTAAGCAATAACGATACACAAA
CTAATGAAAATCAAAGGATTTACTAATACAAACTTCAGTATAGTAATTAATTGGAATGGGAGAGAAAGATGCAAAGTTTCTATTTCTTTTTTTG
TTTTGTTTTGAGACAGTGTCTCATTCTGTTGCCAAGGCAGCAGTGCAGTCGCAGCATCTCAGATCACTGCAAGCTCAGCCTCCTGGGTTCAAGT
GGTTCTTCTGCCTCAGCCTCCCAAGTACCTGGGATTACAAGCATGCACCACCACACCCAGCTAATTTTTGTAATTTTAGTAGAGATGGAGTTTC
ACCATGTTCGCCAGGCTGGTCTCGAACTCCTGGTCTTAAGTAATCCGCCCACCTCTGCCATCAAAGTTTCTGTTTCTTAAGTTGGCTGCCGACT
ACACAGGTTTTCTTTGTAAAATAATTATTTAAATTGTTAATATGCATTACTTATATGCTTTTCATTTACAATGTATTTCACAAGAAAAATAAAA
CAAAGCAAATAAGAAAACAGAACACTTCTCCAGAGATTGATCTTTTTCACTCAAATCTCATTGAGTTATACTGAGGGGGAAAAAGAGTAATCTG
AGTGCCTGGGCCCTGAACAGACACACTAGATCAGTTCCTTCATTCCCACTCAAACACATGCACACACACGCACAAACACACATTTTCTTATATT
TCCTTCAGCAAACCCCCCGCCCCACCAACCAACCCCAAAACACCCTCTGCTCCTTTATGTATATAAATTAGAGCAGATAAATGTCCAGAAGATT
ATAGGCCATCTTTCTATAAACAGCCAATCTTGGACCTGGTGTCTGAATGGGGGATGCCCTGCTGCATTAAAGATGCTCATGTGAACATTTTGTT
GTTTCCCAGAAAAAAAGTTTCCACATTTTAGATTATTTTCACAGGGTGAGAACAATTTTACACCTCCCTTACTTGCTGCAGAATCTTTTTTTTT
TTTTTTTTTTTTTTTTTGAGACAGGTCTTGCTCTATCACTCAAGCTGGAGTGCACTGGCACGATCTCGGCTCACTGCAACCTCCGCCTCCCAGG
TTCAAGCAATTCTTCTGCCTCAGCCTCTCGAGTAGCTGGGATTACAGGTGCGCACCACCATGCCCAGCTAATTTTTTGTATTTTTAGTAGAGAT
GGGGTTTCACCATGATCGCCAAGCTGGTTTCAAACTCCTGACCACAAGTGATCCGCCCACTTCGGCCTCCCAAAGGGCTAGGATTACAGGTGTG
AGCCACCACGCCTGGCTGCTGTGGAATCATTTTTAAAGTGATTGTATCAATTTACAGTGTTCCGAACAGTAAATGGCTACTAGTCTCATCACAC
CCTCATCTTTAAAAAAGTTTTGCTAATTTAATAGACATGATTCTTACATATTGTCTTAATTTGCATTTCTTTGCTTACCAGTGAGGTTGAACAT
TTTTTTCTATGTTACAATCTTTCTTGTATTTCTTCTGTTGTTTTCATTAGAGTAACTCAGAGCAGGATTGGGAATCTGAAAACATGAACATTTG
ATTTTGAGGAAGTCATTGTTTTCCTGTTGTCTTCCTTCCCCCTCTCTGCTTTATTATTTTTTACTTCAGGTTCATTTATAGAAATATCAGAAAA
TACAATTAAGCAAACAGAGTAAATTCCACTTCCCAGAGATAACCACTACCGTTTGTTGTATATCCTTTTAACCTTTTTCTCTGCCAAAAATATT
CACAAATATATCGCTTATTCTTTCTCAGCCTCTTTCCTCATCTATCAAATGGAATACTTGTTCTTCAAGTTGATAATTAACTGGTCGGAGCACA
TGAAAGTATTTTATAAAGTGCACAAATGCAAGTTGCTGCTCTTATGACAAAGAAAAAATGTATTTTATTCTGCCAAGCAGAATGATGACACTTT
GTTTCCTGAACACAGCCTGCTCTCTCCAGTACAATTTCTTCTCTCTAGAATAGCTTCTTGCTTCAATTCCTCCTGCTGAACTACTATCTACTCT
TTTTTTAAAAAAATCAATATTAAAATATACAAAAATTAGTGTAAAGAGCAACCACGTACCCACTATACAGCTTAGGAAAAGAGTCTGGCATTTC
TCACTCCTATGCCTTTATATATACTTCCTTTATTTCCTGTCTCTCTATATATACATACATATATATTTAGTAAGAGCAAAAATACAGTAAAGAT
CAGGGAAGGTCCGGTGGCTCACGCCTGTAATCCCAGCACTTTGGGAGGCCGAGGCGGGCAGATCACGAGGTCAGCAGTTTCACATCAGCTTGAC
CAACATGGTGAAACCATGTCTCTACTAAAAATACAAAAATTAGCCAGGCGTGGTGGCTCATGCGTGTAGTCCCAGCTATTCACGAGGCTGAAGC
AGAAAAATCCCCTGAACCTGGCAGCTGGAGGTTGCCGTGAGCCAAGATTGTGCCACTCACTCCAGCCTGGGCGACAGAGTGACACTCCATCTCG
GGGAAAAAAAAAAATACAGTAAAGGTAAAAAACAGGCAAAACTAAATATATTGCTTAAGCAATAATGATACTCATACAAACTAATGAAAATCAA
AGGATTTACTAATACAAATTTATAGTAGTTAATTGGAAGCATATGTATTATATAAATATACATGTATTATACATATATATATATATGTATATAT
ATAGAAAAAAGCAAATATATGTATATATACATATATATCTATATATGTAAAATTTTTATTTATAAAATGTTTTTGAGCATGAAATGATATATCA
TTGTGGTGTTAATATGCATCTTAGCTGGGCATAGTAGGTCACACCTGTAATCCCAACATTTTGGGAGGCTGAGGCAGGAGGATTTCTTGAGCTC
AGGAGTTCAAGACCAGCCTGGACAGCATAGTGAGACCCCATCTCTAAAAAAAAAAATAAAAATTATCTGGCGTGGTGGCTGATGTCTGTAGTTC
CAGCTACTCAGGAGGCTGAGGTGGGAGGATCACCTGAGCACTGGAGGTCAAGGCTGCAGTGAGCTATGATCATGCCACTGCACTCCAGCCTGGA
TGACAGAGCCAGATCCTGTTTCAAATAAATAAATTAAATTAAGTTAAAAAGTATCTCCCTGATTAGTGGGGAAGTTACCCAACTTTTTTTTTTT
TTTTTTAGACAGAGTCTCAGTCTGTCTCCCACGCTGGCATGCAGTCGCACGATCTTGTCTCACCGCAACCTCCACCTCCTGGGTTCAAGCAATT
CTCCTCCCTCAGTCTCCCGAGTAGCTAGGATTACAGGCATGTGCCACCATACCCCGCTAATTTTTTGTATTTAGTCGAGATGGGGTTTCGCCAT
GTTGGGCAGACTGGTCTCGAACTCCCGACCTCAGGTGATCCTCCCACCTGGGCCTCCCAAAGTACTGGGATTACAGGCGTGTGCCACCGTGCCC
AGCAGTTACACAACTTTTACAAGTTTATTGTTGTTCATGTTGCCTCTTCAGGGAATTTTGTTTATAAAGTACCTATTCTTCAAGGCCCCATTAA
AACACCTCCCTCTCCATAAAGTATTTCCAGATCTCCTCTATCAAGCAGATAGGATTTCCTTTGGACTTCTATAATCCTTTCTTTCTACATCTTG
GGTTTCACTCACTTCTTATTTCATCTTGCATTACAGTCACTTGTGTACTTGTCTAAAGCGCAGTGACTATGTTTAACTCAACTTTATGCCCTTC
TTCATACTAGTACAAAAGTAAACATTTCATAAATCTTTGTTCAATCAAATCCTACTCCTCCCCTGCTTATAAAATCTTAAATCATTTCCTATTG
TCTACAGACGAAAGTAGAATCTCGTTTAATGTAGAATTTAAGACCCTTCCACGGGCTCTAGCATCAACCTTCCTTTCTTGCTCTTCTCCTGGGA
GGACCTTGCTTTCCGCCCCAGATACTAGATTCTTCATCCACAGTAAGGTCCCAGTCCACTAATGTTTGGCTTTTCTCTCTGTAGCTGGGATGGC
AGGTTGGTCCCACCCATTTTAGTTGCCTATAAAATTCTTTTTTTTTTTTTTTTGAGATGGAGTCTTGCTCTGTTGCCCAGGCTGGTATGCAGTG
GCATGATCTTGGCTCACTGCAACCTCCGCCTCCTGTATTCAGGCAATTCTCCTGCCTCAGCCTCCCGAGTAGCTGGGATTATAGGCAACTGCCA
CCATGCCCGGCTAATTTTTGTATTTTTAGAAGAGACGGGGTTTCACCATGTTGGCCAGGCTGGTCTCGAACTCCTGACCTCAGATGATCCGCCC
TCCTCGGCCTCCCAAAGTGCTGGGATTACAGGCGTGAGCCACCGCGCCCGGTCTGAGCCACCGCGCCCGGTCTGAGCCACCACACCCGGTCTAA
TTTTTGTATTTTTAGTAGAGGTGGGGTTTCACCATTTTGGCCAGGCTGGTTTTGAACTCCTGACCTCGTGATCTGCCCGCCTCGGCCTCCCAAA
GTGCTGGGATTACAGGCTTGAGCCACTGCGTCCAGCCCAAACTCTTTAAGAGAAAATCTCTTGGGATCTTGAAGCCAGAAGCTTGCTTTTATTA
CTGAGTTTGCCCTGAATCTGTCACTTATGTACTAAAAATACATAAATTTCCTTATCCAGGTGACTTGGGCTCAACTGAACGAAACCTGAAGCAT
CTAAATGTTAGCACCTAAGCCTGAGTCTAGGTGCCAGCATAGCCTGTCTTCCCTCTCCAGAGTCAGAGCCATGTTACAAACAGAATATATGGAC
ACACGTCGAACCCCACATTGTATCTCTCTAATTTATTCAATTTAATTCAGTAAACACTCATTACCTCTTATATCCAGCCCTAGTTGAGGGTGCT
GGGGATACAGGTATAAACATGACAGTTCTCTTGAGGAGCACAGAGTGGTTGTGGGAAACAGACATACAGACACACAGTTTCTTTCTTTCTTTTT
TCTTTTTTTTTTTTTTTTTGACACAGAGTCTCACTCTGTCCTTCAGGCCGGAATGCAGTGAGGTGATCTCAGCTCACCACAACCTCCACCTCCT
GGGTTCAAGCGATTCTCCTGCCTCTCACTCCCCAGTAGCTGGGATTACAGTAATGCGCCACCACACCCACCTAATTTTTGTATTTTTAGTAGAG
ACAGGCTTTTGCCATGTTGGCCAGGCTGGTCTCAAACTCCTGAACTCAGGTGATCCGCCTGCCTCGGCCTCCCAAAGTGCTGGGATTACAGGTG
TGAGCCACCATGCCCGGCCCAGACACATAGTTTCAATACAAGAAGTATATGTCAGCCAGAGAAGGGCTATCTTGAGAGTCAAGGAAGCCTCCCT
GGAGGAGGTATCACTTATGCCAGTTGGTACATGAATGAGTAAATGCAAACAAGCCATGATACTGAAATAAAGCTTTGTTTAATTAACCTTCACA
TGTAAATTCAGCCACAGAAGACAXTCTGATACATGGGGGCATTGCCAGTGATTCCACGAAACTGGATAATGTCATTGATGCTTGTGGTTGAGAG
AGCTTGTTTCTGTATTGTAAGGAAGGTGGTACAACCTGGCCTTTCTTTTAAAAAAAAAAAAAAATTCAAACTGGACATGTGTCAATGAACTGAC
TGGAGTCTTAGGGAGCAACATTGCTCCATAGCTATATGTCTCTCCCGTTTTTTCCTGTGTTAGTCTTTTTTTTTAATTTTTTTAAAATTTTTTT
GAGACAGAGTCTTGCTCTGTTGCCCAGGCTGGAGTGCAGTGGTGTGATCTTGGCTCGCTGCAAGCTCCGCCTCCCGGGTTCAAGCAATTCTCCT
GCCTCAGCTTCCCAAATAGCTGGGACTACAGATGCATGCCACCATGCCTGGCTAATTTTTGTATTTTTAGTAGAGACAGAATTTCACCATGTTG
GCTACACTGGTTTGGACCTCCTGACCTGAGGGGTTCCACCTGCCTCGGCCTCTCAAAGTGCTGAGATTACAGGAGTGAGCCACCTTGTCTAGCT
CTTTTTTTTTTTTTCTTTTGACACAAGTTCTTGCTCTGTCTCCCAGGCTGGAACGCAGTGGTGCAATCACAGCTCCCTGCAGCCTCGACCTCCC
CAGCTCAAGTGATCTTCCCACCTCAGCCTCCCCAGTAGCTGGGGCCACAGGCATGCACCACTACGCCCAGCTAATTTTTTTTATTTTTTGTAGA
GATGGTGTCTCACCATGTTGCACAGGCTGGTATCCAACTGCTGGGCTCAAGCAATCTTCCTGCCATGGCCTCCCAAAGTGCTAGGATTACAGGC
GTGAGCCACTGTGCCCACGTGTTAGTCTTTACATAACTGAATAGTGACTGAAGTCTTGCAGTTTCTACCTTGCCCTGCACTGGCATTATAAGTT
GTAGTAGAAATGTCACTCATTGCTTGTAATCTCATGAGAGGCCTAGTTAGATTTTTCTGTACTCTACTTCCAGAGCACCTTATAGGAAGGTGAC
TTTCCATAAAGGATGTGAGCTTTCTACCACCCAAATATCAGTCGTAAAGAAAATGAGGAGTGATAAAGAAAGAAGGTATTCAGAGGTGAAGCCA
GCTGGGCTTCTAGGTTGGGTGGGGACTTGGAAAACTTTTCTGTCTAGCTAGAGGATTAAAAACGCACCAATCAGCACTCTTTGTCTAGCTAAAG
TATTGTAAATGCACCAATCAGCACACTGTAAAAACGCACCAATCAGCACTCTGTGTCTAGCTAAAGGACTGTAAACGCACCAGTCAGCACTCTG
TAAAATGGACTAATCAGCAGGATGTGGGTGGGGCCAAATAAGGCAATAAAAGCTGGCCAGCGAAGCCAGCAGCAGGCAACCCACTCGGGTGCCC
TTCCATGCTGTGGAAGCTTTGTTCTTTCGCTCTTCACAGTAAATCTTGCTGCTGCTCACTCTGGGTCCGCACTACCTTCATGAGCTGTAACACT
CAATGCGAGGGTCTGCGGCTGCATTCCTGAAGTCACCAAGACCACAAACCCACCGGGAGGAACAAACAACTCCAGATGAGCCACCTTTAAGAGC
TGTAACACTCACTGCGAAGGTGTGCGGCTTCACTCCTGAAGTCAGTGACACCACGAACCCACTGGAAGGAAGAAACTCCGGACACATCTGAACA
TCTGAAGGAACAAACTCCGGACACACCATCTTTAAGAGCTGTAACGCTCACCGTGAAGGTCCGTGGTTTCATTCTTGAAGTCAGCGAGACCAAG
CATCCACCAAAAGGAATAAATTCCAGACCCACTATTTACAGCCCAGTGTCCACCCATTCCCCATGTAGCAACCCCTGGTACCTGAGGAAAAAGG
TCACAGCCTCTGCCAATGGTGGGGCATCTTTCCGAAGAGGTTCCACTTGGACTTTTTGAAACATCATGTGGCTGTGCTGTTTGAGAAGCCTGGG
GTGAAATTAACTATACAAGAATCAGGGCATGATCACACACGCACACACCTTTGCAATTTTTCTGGCTTTGTTTACACTGTTACTGCAACTTGTG
CCCTTTCTCACCCTTTTCATAGGTCTTCCCTGCAAAGTCTTTTTTTTTTTTTTTTTTTGAGACGGAGTCTCGCTCTGTCGTTCAGGCTGGAGTG
CAGTGGCGTGATCTCGGCTCACTGCAACCTCCGCCTCCTGGGTTCACGCCATTCTCCCGCCTCAGCCTCCCGAGTAGCTGGGACTACAGGCACC
TGCTACCTGGCGCAGCTAATTTGTGTATTTTTAATAGAGACGGGTTTCACCGTGTTAACGAGGATGGTCTCCATCTCTTGACCTCATGATCTGC
CCGCCTCAGCCTCCCAAAGTGTTAGGATTACAGGCCTGAGCCACCGCACCCGACCCATCAACTGTTCTCTTATCTGTGCTCCTAGATCTCTATC
TTGATGTCTGAAAGCTTCTTGAGAGTGCTTTGTCAGGTCTATGTCCATCACATTCTTTGGTGGATGTGGCCTCACAAATCTTTCAACTCAGCCC
TCTAGGTCCTCATGTGCATTAGTGGAGAATAGCCTTCACATATGTGGGCTTTGGGTCCAATCCAGTTTGAATCCTGAGTATGTCACTAACTAGT
TGTGTAATTTTTGGAACATTGTTAACCTTTCTGATTTCAGTTTCCTTATCCTTAAATTGAAAATTCTAGTAGTTCCTACCTCATAGGATTGTTG
TGAAGGTCAAATGAGATCAAATACTTACAACTGGACCTAAGGGGCACTCATTATGAGTTAGCTGTTGTTACTAGTAACCCATTGTCATCCCTGT
AGATGTTTCAGGGGTTTGCCGTGGGGAACCCTGCCATGCAGGCTTGTCTCAGGTTCTCACCCTCTGATGGGGTCTGGTGTTCCCTGCCTCGTTT
ACCACTCTTTCCCCATACTTTGGTCTGACTCTCCAATGAGCTCATGATTCCTATGAGCGATTTCTGTCTCTTGATGAATCTTTAGATTTGTCCT
TTGTCTTAGGCCTTGAGACTGCAGTAGAACCTTGTGGCCTATTCTAACCCCAGCCCGCAAACCAATGCCCCAACTCCATTCTGGGCTGACTTCC
TGGCTTCCTCCCACAACTTCCGGGTCTCTGTTTTTATATATTGTTCCTAGACTAGGATATGCGTGTTCACAAATGTTTGTTGAAAGGTTTTCAG
AGCCAATTACGCATATTTAAGAAGTGAATGAACACCTGCTCATTGATATGCTGTCCATAAACTAGGACACGGTGTTGAATGATGATTTGACAGA
GATTTTAGAATCAGAGAGATGGAAGCTTGATCCTAACTCTGAATCTTATGGCCTTAAGCAAAATATCTAACCTCTTTGTGCCTGTTTTCTCATT
TGTAAAATGAGGATAATTATATTTCCTCATAGGGTTATTGTGAAGATTAAATGAGACAATATAAATAAAGCACCTAGCATTTTCTGGAACATGG
TAAGCAGTTGGTAAATAGCTGCAACTACCAACAGGTAGTTCAAAACTTTTCCTCCATCTGAAAAACCAAGAGTTGAAGAAATGGCTGAAGTTGG
AATATCTGGCAGGAGCTGTGCTTAAACACAAGATCCGGAATATCTGAATATCTTTAGGACAAGATGAAATAGGTTTCTCTCAAATGAGTTGGTT
AAGGAGTTAAGCAGTGATAGGTAACACTTGAAGTTTCACAAAGATGAAAACTGGTACTTGACAGACAGCCACTTGATGGTATTCTGAAGTTTAC
TGACCCTTTGTTCTATTTCTAGAACAAGGTGTTCTATCTCTTTGCCCAAGGTGTTCAAAGTATAGAGAAACAAAGAAGATCAGTAATTACATAG
ACCAAATGCAGTATCGGATTTATTACGAACTACATCTGCTCAAAAGACTCATCAACTATATGGAGTTAGTCCTATCAGAGAGAAGCAGGGTATC
CAGCTAGCTCATAGGCCTTTTTATACAAAAGTTTACCATTAGCATGTTTGGTAACATTACTTATAACCCACCCCAACCCCCTCCCCACACACTC
AAGTTTCCTTAAGTGTATGTATTAGTTTTCTATTGAAGTACAGGCAAGCTTGACTGGGTTCTTTGCTCAGAGTCTTACAAGGTCAAGATTAAAA
TATCCACTAGATTAAGCACTTATCTTCAGACTGTGAGGACAATCTCCTTCCAAGCTCATTTAGGCTATAGGTAGAATCAAGTTCCTTGCAGTTA
TAGGTCTGAGGTCCCTGTTTCCTTGCTGACTATCAGTCAGGGGCCACTCTTAGCTTCTCGAGGCTGCTCTCAAGTCCTTTCCATGTGGACCCCT
CCATATTCAATACCAGCAATGGCCCATTGAACCATCCCATGCTTTCAGTCTCTGACTTCTTTTGCCAGCTAAAGGAATCTCTCTACTTATAAAT
GGCTCATGTGATGACCAGGATAATTTCCCCATCTTAAGATCAACTGTTTAGTGCCTTAATTGCATCTGCAAAATCCCTTCAAAATAGTGCCTAG
ATTAGTGTTTGATTGAATAGCCAAGGATTGGCAATCTTGGGCAGTCATCTCAGAATTCTGCCTACACACCATGGTTACAATTTCCTGAAAAGCT
ATCCTGTGAGGCCTGACAGTTATAGTACAAGCATTGGATAAACATTCCCATTTCAGAATCGAGAAATCAGCCAACAAGAAAGGTGCTACAGGCC
CCATGCAAGTTTGAAAGCTGGAAGGGCAGTCATTAGATCTTAAAGCTCTAACATAATCTTTGACTCCATCTCCCATATGCTCGGCATACCGGTG
CAGTGCGTGCGCTCCCAAGGCATGGAGCAGCTCTGTCCCTGTGGCTTTCCAGGATTCATCACCCATGACTGCTCTCATGGGCTCCAGTTGAGTG
CCTGTGGCTTTGCCAGGCACAGGGTGCAAGCTGCTGTTGGATCTACCATTCTTGGATCTGGAGGATGGTGGCCCTCTTCTCACAGCTCCACTAG
GCAGTGTGCCCCATTGGGAACTCTGTGTCGGCAATGTGTATTAATCTGTTCTCACACTGCTAATAAAGACATACCCGAGACTGGGTAATTTATA
AAGCAAAGGGGTTTAATTGACTCACAGTTCCACCGTGGGGTGGGGCTCACAACCATGGCAGAAGGCAAATGAGGGGCAAAGTCAAATCTTATAT
GGCAGCAGACAAGAGAGCTTGTGCAGTGCAACTCCCATTTATAAAACCATCAGATCTCAGGGAACTTATTCACTACCACTAGAATAGTATGAGG
GAAACCACTGCCACGATTTAATTATCTCCACCTGGCCCCACCTTTCACACGTGGAGATTGTTACAAATCAAGGTGAGATTTGTGTGGGGACACA
GCCAAACCATATCAGTGTGCCAACCTTACAATTCCCCTTGGCATTGACCTAGTAGAGGTTCTCTGTGAAGGCTCCACCCCTGCAGCAGTCTTCT
GCCTGGACACCCAGACTTGTCCATACATCCTCTGAAATCAAGGTGGAGGGTGGCAAACCTCAAGTCTTGTGCTCTGTACACCGCAGCCTTAACA
CTACATGGAAGCCACCAAGGCTTTTGGCTTCCACCATCTGGAACTGCACCCCAAGCTGTACTTGGGCCCCTTTGAGCTGTGGGTAGAGGTGGAG
CAGCCTGGATGTGGGAAGAAGTGTCCCAAGGCTGTGCAGGGCAGCAGGGCCCTGGGCTTGGTCCAGGAAACCATTCTTCCCTCCTAGCCCACTG
GGCATATAATGGGAGAGGCTGCCATGAAGGTCTCTAAAATGCCTTTGAGGTCTTTTTCCCATTGTCTTTGGAATTAGTTCTTGGGTCCTTTTTA
GTCAAGCAAATTTGTCTAGCAAATGGTGGCTCTCCACAGCCTGCTTGAATTCCTCTCCTGAAAAAGCTTTTTCTTTCTTTGCCACATAGCCAGG
CTGCAAATTTTTCAAACTTTTATGCTGTGCTTCCTGTTTAAATATAAATTCCAACTTTAAGTCATTTCTTTGCTCCCACATTTAAGTTAGGCTG
TCAGAAGCAGCCATGCCACAACTTCAATGCTTTGCTGCTGAGAAATTTCTTCTGCTGGCCAGGCGTGGTGGCTTATGCCTGTAATCCCAGCACT
TTGGGAGGCTGAGGTGGGCGGATCACCTGAGGTCAGGAGTTCGAGACCAGCCTGGTCAACATGGTGAAATCTCATCTCTATTAAAAATACAAAA
ATTAGCTGGGCATGGTGGTGGGTGCCTGTAATCCCTGCTACTCAGGAGGCTGAGGCATGAGAATTGCTTGAACCCGGGAGGCAGAGGTTGCAGT
GAGCCAAGATGCACCACTGCACTCCAGCCTGGGTGACAGAGCGAGACTCAGTCTCAAAAAAAAAAAAAAAAAATTCTTCTGTCAGACACTTAGA
TCATCACCCTTTAGTTAAAAGTTCCACAGATCCCTAGGGCGTGGACAGAACACAGCCAAGCTCTTTGCTAAGACACAACAAATGTGACCTTTGT
TTTAGTTCTCAAGAAGTTTCTCACTTCCATCTGAGACCTTGTCAGCCTGGCCTTCATTGTCCATATCACTATCAGCATTTTGGTTGAAACTATT
TAACCAGTCTCTAAGAAGTTCCAAACTTTCCCTCACCTTCCTATCTTCTTCTGAGCCCTCCAAACTCTTCCAACCTATACTTGTTACCCAGTTG
HUMAN SEQUENCE—mRNA
GGTATTAAAACTGATCTTTTGACATTTTTGACAATGTTCTTATAAATTACTTTCTTTTTTATCATATATGGATCGCATCAAGCACAGAGTAAGA
TAGAGTGCACAGCAAAGGGGATCTGCCCCTCCTATCTGTCCAATACCCCACAGGTTTTGGTGATAATCTTGGGCAATGTTCCAGTCAAACCTGC
CTCCCACTTCTCACTAAAGTTAGTGAACATGTGACCCACATTCCCCAAATAAGAGCCTCTTATAAACTCCATTCTTGGCTTTTTCATTCATAGA
GATAGCTATTTTATGAGACATAGATAAAGCATTTTTTAGTGATGTCCACGATGCCTTTTTTCTTAATTATTAACTTCTCAAAACATAAACACAT
TGGAGGCACTTAATAAAGGGAGCTGTACGTACCGCCGTCCGCGCCTCCAAGGTTTCACGGCTTCCTCAGCAGAGACTCGGGCTCGTCCGCCATG
TCCGCCGCAGACGAGGTTGACGGGCTGGGCGTGGCCCGGCCGCACTATGGCTCTGTCCTGGATAATGAAAGACTTACTGCAGAGGAGATGGATG
AAAGGAGACGTCAGAACGTGGCTTATGAGTACCTTTGTCATTTGGAAGAAGCGAAGAGGTGGATGGAAGCATGCCTAGGGGAAGATCTGCCTCC
CACCACAGAACTGGAGGAGGGGCTTAGGAATGGGGTCTACCTTGCCAAACTGGGGAACTTCTTCTCTCCCAAAGTAGTGTCCCTGAAAAAAATC
TATGATCGAGAACAGACCAGATACAAGGCGACTGGCCTCCACTTTAGACACACTGATAATGTGATTCAGTGGTTGAATGCCATGGATGAGATTG
GATTGCCTAAGATTTTTTACCCAGAAACTACAGATATCTATGATCGAAAGAACATGCCAAGATGTATCTACTGTATCCATGCACTCAGTTTGTA
CCTGTTCAAGCTAGGCCTGGCCCCTCAGATTCAAGACCTATATGGAAAGGTTGACTTCACAGAAGAAGAAATCAACAACATGAAGACTGAGTTG
GAGAAGTATGGCATCCAGATGCCTGCCTTTAGCAAGATTGGGGGCATCTTGGCTAATGAACTGTCAGTGGATGAAGCCGCATTACATGCTGCTG
TTATTGCTATTAATGAAGCTATTGACCGTAGAATTCCAGCCGACACATTTGCAGCTTTGAAAAATCCGAATGCCATGCTTGTAAATCTTGAAGA
GCCCTTGGCATCCACTTACCAGGATATACTTTACCAGGCTAAGCAGGACAAAATGACAAATGCTAAAAACAGGACAGAAAACTCAGAGAGAGAA
AGAGATGTTTATGAGGAGCTGCTCACGCAAGCTGAAATTCAAGGCAATATAAACAAAGTCAATACATTTTCTGCATTAGCAAATATCGACCTGG
CTTTAGAACAAGGAGATGCACTGGCCTTGTTCAGGGCTCTGCAGTCACCAGCCCTGGGGCTTCGAGGACTGCAGCAACAGAATAGCGACTGGTA
CTTGAAGCAGCTCCTGAGTGATAAACAGCAGAAGAGACAGAGTGGTCAGACTGACCCCCTGCAGAAGGAGGAGCTGCAGTCTGGAGTGGATGCT
GCAAACAGTGCTGCCCAGCAATATCAGAGAAGATTGGCAGCAGTAGCACTGATTAATGCTGCAATCCAGAAGGGTGTTGCTGAGAAGACTGTTT
TGGAACTGATGAATCCCGAAGCCCAGCTGCCCCAGGTGTATCCATTTGCCGCCGATCTCTATCAGAAGGAGCTGGCTACCCTGCAGCGACAAAG
TCCTGAACATAATCTCACCCACCCAGAGCTCTCTGTCGCAGTGGAGATGTTGTCATCGGTGGCCCTGATCAACAGGGCATTGGAATCAGGAGAT
GTGAATACAGTGTGGAAGCAATTGAGCAGTTCAGTTACTGGTCTTACCAATATTGAGGAAGAAAACTGTCAGAGGTATCTCGATGAGTTGATGA
AACTGAAGGCTCAGGCACATGCAGAGAATAATGAATTCATTACATGGAATGATATCCAAGCTTGCGTGGACCATGTGAACCTGGTGGTGCAAGA
GGAACATGAGAGGATTTTAGCCATTGGTTTAATTAATGAAGCCCTGGATGAAGGTGATGCCCAAAAGACTCTGCAGGCCCTACAGATTCCTGCA
GCTAAACTTGAGGGAGTCCTTGCAGAAGTGGCCCAGCATTACCAAGACACGCTGATTAGAGCGAAGAGAGAGAAAGCCCAGGAAATCCAGGATG
AGTCAGCTGTGTTATGGTTGGATGAAATTCAAGGTGGAATCTGGCAGTCCAACAAAGACACCCAAGAAGCACAGAAGTTTGCCTTAGGAATCTT
TGCCATTAATGAGGCAGTAGAAAGTGGTGATGTTGGCAAAACACTGAGTGCCCTTCGCTCCCCTGATGTTGGCTTGTATGGAGTCATCCCTGAG
TGTGGTGAAACTTACCACAGTGATCTTGCTGAAGCCAAGAAGAAAAAACTGGCAGTAGGAGATAATAACAGCAAGTGGGTGAAGCACTGGGTAA
AAGGTGGATATTATTATTACCACAATCTGGAGACCCAGGAAGGAGGATGGGATGAACCTCCAAATTTTGTGCAAAATTCTATGCAGCTTTCTCG
GGAGGAGATCCAGAGTTCTATCTCTGGGGTGACTGCCGCATATAACCGAGAACAGCTGTGGCTGGCCAATGAAGGCCTGATCACCAGGCTGCAG
GCTCGCTGCCGTGGATACTTAGTTCGACAGGAATTCCGATCCAGGATGAATTTCCTGAAGAAACAAATCCCTGCCATCACCTGCATTCAGTCAC
AGTGGAGAGGATACAAGCAGAAGAAGGCATATCAAGATCGGTTAGCTTACCTGCGCTCCCACAAAGATGAAGTTGTAAAGATTCAGTCCCTGGC
AAGGATGCACCAAGCTCGAAAGCGCTATCGAGATCGCCTGCAGTACTTCCGGGACCATATAAATGACATTATCAAAATCCAGGCTTTTATTCGG
GCAAACAAAGCTCGGGATGACTACAAGACTCTCATCAATGCTGAGGATCCTCCTATGGTTGTGGTCCGAAAATTTGTCCACCTGCTGGACCAAA
GTGACCAGGATTTTCAGGAGGAGCTTGACCTTATGAAGATGCGGGAAGAGGTTATCACCCTCATTCGTTCTAACCAGCAGCTGGAGAATGACCT
CAATCTCATGGATATCAAAATTGGACTGCTAGTGAAAAATAAGATTACGTTGCAGGATGTGGTTTCCCACAGTAAAAAACTTACCAAAAAAAAT
AAGGAACAGTTGTCTGATATGATGATGATAAATAAACAGAAGGGAGGTCTCAAGGCTTTGAGCAAGGAGAAGAGAGAGAAGTTGGAAGCTTACC
AGCACCTGTTTTATTTATTGCAAACCAATCCCACCTATCTGGCCAAGCTCATTTTTCAGATGCCCCAGAACAAGTCCACCAAGTTCATGGACTC
TGTAATCTTCACACTCTACAACTACGCGTCCAACCAGCGAGAGGAGTACCTGCTCCTGCGGCTCTTTAAGACAGCACTCCAAGAGGAAATCAAG
TCGAAGGTAGATCAGATTCAAGAGATTGTGACAGGAAATCCTACGGTTATTAAAATGGTTGTAAGTTTCAACCGTGGTGCCCGTGGCCAGAATG
CCCTGAGACAGATCTTGGCCCCAGTCGTGAAGGAAATTATGGATGACAAATCTCTCAACATCAAAACTGACCCTGTGGATATTTACAAATCTTG
GGTTAATCAGATGGAGTCTCAGACAGGAGAGGCAAGCAAACTGCCCTATGATGTGACCCCTGAGCAGGCGCTAGCTCATGAAGAAGTGAAGACA
CGGCTAGACAGCTCCATCAGGAACATGCGGGCTGTGACAGACAAGTTTCTCTCAGCCATTGTCAGCTCTGTGGACAAAATCCCTTATGGGATGC
GCTTCATTGCCAAAGTGCTGAAGGACTCGTTGCATGAGAAGTTCCCTGATGCTGGTGAGGATGAGCTGCTGAAGATTATTGGTAACTTGCTTTA
TTATCGATACATGAATCCAGCCATTGTTGCTCCTGATGCCTTTGACATCATTGACCTGTCAGCAGGAGGCCAGCTTACCACAGACCAACGCCGA
AATCTGGGCTCCATTGCAAAAATGCTTCAGCATGCTGCTTCCAATAAGATGTTTCTGGGAGATAATGCCCACTTAAGCATCATTAATGAATATC
TTTCCCAGTCCTACCAGAAATTCAGACGGTTTTTCCAAACTGCTTGTGATGTCCCAGAGCTTCAGGATAAATTTAATGTGGATGAGTACTCTGA
TTTAGTAACCCTCACCAAACCAGTAATCTACATTTCCATTGGTGAAATCATCAACACCCACACTCTCCTGTTGGATCACCAGGATGCCATTGCT
CCGGAGCACAATGATCCAATCCACGAACTGCTGGACGACCTCGGCGAGGTGCCCACCATCGAGTCCCTGATAGGGGAAAGCTCTGGCAATTTAA
ATGACCCAAATAAGGAGGCACTGGCTAAGACGGAAGTGTCTCTCACCCTGACCAACAAGTTCGACGTGCCTGGAGATGAGAATGCAGAAATGGA
TGCTCGAACCATCTTACTGAATACAAAACGTTTAATTGTGGATGTCATCCGGTTCCAGCCAGGAGAGACCTTGACTGAAATCCTAGAAACACCA
GCCACCAGTGAACAGGAAGCAGAACATCAGAGAGCCATGCAGAGACGTGCTATCCGTGATGCCAAAACACCTGACAAGATGAAAAAGTCAAAAT
CTGTAAAGGAAGACAGCAACCTCACTCTTCAAGAGAAGAAAGAGAAGATCCAGACAGGTTTAAAGAAGCTAACAGAGCTTGGAACCGTGGACCC
AAAGAACAAATACCAGGAACTGATCAACGACATTGCCAGGGATATTCGGAATCAGCGGAGGTACCGACAGAGGAGAAAGGCCGAACTAGTGAAA
CTGCAACAGACATACGCTGCTCTGAACTCTAAGGCCACCTTTTATGGGGAGCAGGTGGATTACTATAAAAGCTATATCAAAACCTGCTTGGATA
ACTTAGCCAGCAAGGGCAAAGTCTCCAAAAAGCCTAGGGAAATGAAAGGAAAGAAAAGCAAAAAGATTTCTCTGAAATATACAGCAGCAAGACT
ACATGAAAAAGGAGTTCTTCTGGAAATTGAGGACCTGCAAGTGAATCAGTTTAAAAATGTTATATTTGAAATCAGTCCAACAGAAGAAGTTGGA
GACTTCGAAGTGAAAGCCAAATTCATGGGAGTTCAAATGGAGACTTTTATGTTACATTATCAGGACCTGCTGCAGCTACAGTATGAAGGAGTTG
CAGTCATGAAATTATTTGATAGAGCTAAAGTAAATGTCAACCTCCTGATCTTCCTTCTCAACAAAAAGTTCTACGGGAAGTAATTGATCGTTTG
CTGCCAGCCCAGAAGGATGAAGGAAAGAAGCACCTCACAGCTCCTTTCTAGGTCCTTCTTTCCTCATTGGAAGCAAAGACCTAGCCAACAACAG
CACCTCAATCTGATACACTCCCGATGCCACATTTTTAACTCCTCTCGCTCTGATGGGACATTTGTTACCCTTTTTTCATAGTGAAATTGTGTTT
CAGGCTTAGTCTGACCTTTCTGGTTTCTTCATTTTCTTCCATTACTTAGGAAAGAGTGGAAACTCCACTAAAATTTCTCTGTGTTGTTACAGTC
TTAGAGGTTGCAGTACTATATTGTAAGCTTTGGTGTTTGTTTAATTAGCAATAGGGATGGTAGGATTCAAATGTGTGTCATTTAGAAGTGGAAG
CTATTAGCACCAATGACATAAATACATACAAGACACAGAACTAAAATGTCATGTTATTAACAGTTATTAGGTTGTCATTTAAAAATAAAGTTCC
TTTATATTTCTGTCCCATCAGGAAAACTGAAGGATATGGGGAATCATTGGTTATCTTCCATTGTGTTTTTCTTTATGGACAGGAGCTAATGGAA
GTGACAGTCATGTTCAAAGGAAGCATTTCTAGAAAAAAGGAGATAATGTTTTTAAATTTCATTATCAAACTTGGGCAATTCTGTTTGTGTAACT
CCCCGACTAGTGGATGGGAGAGTCCCATTGCTAAAATTCAGCTACTCAGATAAATTCAGAATGGGTCAAGGCACCTGCCTGTTTTTGTTGGTGC
ACAGAGATTGACTTGATTCAGAGAGACAATTCACTCCATCCCTATGGCAGAGGAATGGGTTAGCCCTAATGTAGAATGTCATTGTTTTTAAAAC
TGTTTTATATCTTAAGAGTGCCTTATTAAAGTATAGATGTATGTCTTAAAATGTGGGTGATAGGAATTTTAAAGATTTATATAATGCATCAAAA
GCCTTAGAATAAGAAAAGCTTTTTTTAAATTGCTTTATCTGTATATCTGAACTCTTGAAACTTATAGCTAAAACACTAGGATTTATCTGCAGTG
TTGCAGGGAGATAATTCTGCCTTAAATTGTCTAAAACAAAAACAAAACCAGCCAACCTATGTTACACGTGAGATTAAAACCAATTTTTTCCCCA
TTTTTTCTCCTTTTTTCTCTTGCTGCCCACATTGTGCCTTTATTTTATGAGCCCCAGTTTTCTGGGCTTAGTTTAAAAAAAAAATCAAGTCTAA
ACATTGCATTTAGAAAGCTTTTGTTCTTGGATAAAAAGTCATACACTTTAAAAAAAAAAAAAAAACTTTTTCCAGGAAAATATATTGAAATCAT
GCTGCTGAGCCTCTATTTTCTTTCTTTGATGTTTTGATTCAGTATTCTTTTATCATAAATTTTTAGCATTTAAAAATTCACTGATGTACATTAA
GCCAATAAACTGCTTTAATGAATAACAAACTATGTAGTGTGTCCCTATTATAAATGCATTGGAGAAGTATTTTTATGAGACTCTTTACTCAGGT
GCATGGTTACAGCCACAGGGAGGCATGGAGTGCCATGGAAGGATTCGCCACTACCCAGACCTTGTTTTTTGTTGTATTTTGGAAGACAGGTTTT
TTAAAGAAACATTTTCCTCAGATTAAAAGATGATGCTATTACAACTAGCATTGCCTCAAAAACTGGGACCAACCAAAGTGTGTCAACCCTGTTT
CCTTAAAAGAGGCTATGAATCCCAAAGGCCACATCCAAGACAGGCAATAATGAGCAGAGTTTACAGCTCCTTTAATAAAATGTGTCAGTAATTT
TAAGGTTTATAGTTCCCTCAACACAATTGCTAATGCAGAATAGTGTAAAATGCGCTTCAAGAATGTTGATGATGATGATATAGAATTGTGGCTT
TAGTAGCACAGAGGATGCCCCAACAAACTCATGGCGTTGAAACCACACAGTTCTCATTACTGTTATTTATTAGCTGTAGCATTCTCTGTCTCCT
CTCTCTCCTCCTTTGACCTTCTCCTCGACCAGCCATCATGACATTTACCATGAATTTACTTCCTCCCAAGAGTTTGGACTGCCCGTCAGATTGT
TTCTGCACATAGTTGCCTTTGTATCTCTGTATGAAATAAAAGGTCATTTGTTC
HUMAN SEQUENCE—CODING
ATGTCCGCCGCAGACGAGGTTGACGGGCTGGGCGTGGCCCGGCCGCACTATGGCTCTGTCCTGGATAATGAAAGACTTACTGCAGAGGAGATGG
ATGAAAGGAGACGTCAGAACGTGGCTTATGAGTACCTTTGTCATTTGGAAGAAGCGAAGAGGTGGATGGAAGCATGCCTAGGGGAAGATCTGCC
TCCCACCACAGAACTGGAGGAGGGGCTTAGGAATGGGGTCTACCTTGCCAAACTGGGGAACTTCTTCTCTCCCAAAGTAGTGTCCCTGAAAAAA
ATCTATGATCGAGAACAGACCAGATACAAGGCGACTGGCCTCCACTTTAGACACACTGATAATGTGATTCAGTGGTTGAATGCCATGGATGAGA
TTGGATTGCCTAAGATTTTTTACCCAGAAACTACAGATATCTATGATCGAAAGAACATGCCAAGATGTATCTACTGTATCCATGCACTCAGTTT
GTACCTGTTCAAGCTAGGCCTGGCCCCTCAGATTCAAGACCTATATGGAAAGGTTGACTTCACAGAAGAAGAAATCAACAACATGAAGACTGAG
TTGGAGAAGTATGGCATCCAGATGCCTGCCTTTAGCAAGATTGGGGGCATCTTGGCTAATGAACTGTCAGTGGATGAAGCCGCATTACATGCTG
CTGTTATTGCTATTAATGAAGCTATTGACCGTAGAATTCCAGCCGACACATTTGCAGCTTTGAAAAATCCGAATGCCATGCTTGTAAATCTTGA
AGAGCCCTTGGCATCCACTTACCAGGATATACTTTACCAGGCTAAGCAGGACAAAATGACAAATGCTAAAAACAGGACAGAAAACTCAGAGAGA
GAAAGAGATGTTTATGAGGAGCTGCTCACGCAAGCTGAAATTCAAGGCAATATAAACAAAGTCAATACATTTTCTGCATTAGCAAATATCGACC
TGGCTTTAGAACAAGGAGATGCACTGGCCTTGTTCAGGGCTCTGCAGTCACCAGCCCTGGGGCTTCGAGGACTGCAGCAACAGAATAGCGACTG
GTACTTGAAGCAGCTCCTGAGTGATAAACAGCAGAAGAGACAGAGTGGTCAGACTGACCCCCTGCAGAAGGAGGAGCTGCAGTCTGGAGTGGAT
GCTGCAAACAGTGCTGCCCAGCAATATCAGAGAAGATTGGCAGCAGTAGCACTGATTAATGCTGCAATCCAGAAGGGTGTTGCTGAGAAGACTG
TTTTGGAACTGATGAATCCCGAAGCCCAGCTGCCCCAGGTGTATCCATTTGCCGCCGATCTCTATCAGAAGGAGCTGGCTACCCTGCAGCGACA
AAGTCCTGAACATAATCTCACCCACCCAGAGCTCTCTGTCGCAGTGGAGATGTTGTCATCGGTGGCCCTGATCAACAGGGCATTGGAATCAGGA
GATGTGAATACAGTGTGGAAGCAATTGAGCAGTTCAGTTACTGGTCTTACCAATATTGAGGAAGAAAACTGTCAGAGGTATCTCGATGAGTTGA
TGAAACTGAAGGCTCAGGCACATGCAGAGAATAATGAATTCATTACATGGAATGATATCCAAGCTTGCGTGGACCATGTGAACCTGGTGGTGCA
AGAGGAACATGAGAGGATTTTAGCCATTGGTTTAATTAATGAAGCCCTGGATGAAGGTGATGCCCAAAAGACTCTGCAGGCCCTACAGATTCCT
GCAGCTAAACTTGAGGGAGTCCTTGCAGAAGTGGCCCAGCATTACCAAGACACGCTGATTAGAGCGAAGAGAGAGAAAGCCCAGGAAATCCAGG
ATGAGTCAGCTGTGTTATGGTTGGATGAAATTCAAGGTGGAATCTGGCAGTCCAACAAAGACACCCAAGAAGCACAGAAGTTTGCCTTAGGAAT
CTTTGCCATTAATGAGGCAGTAGAAAGTGGTGATGTTGGCAAAACACTGAGTGCCCTTCGCTCCCCTGATGTTGGCTTGTATGGAGTCATCCCT
GAGTGTGGTGAAACTTACCACAGTGATCTTGCTGAAGCCAAGAAGAAAAAACTGGCAGTAGGAGATAATAACAGCAAGTGGGTGAAGCACTGGG
TAAAAGGTGGATATTATTATTACCACAATCTGGAGACCCAGGAAGGAGGATGGGATGAACCTCCAAATTTTGTGCAAAATTCTATGCAGCTTTC
TCGGGAGGAGATCCAGAGTTCTATCTCTGGGGTGACTGCCGCATATAACCGAGAACAGCTGTGGCTGGCCAATGAAGGCCTGATCACCAGGCTG
CAGGCTCGCTGCCGTGGATACTTAGTTCGACAGGAATTCCGATCCAGGATGAATTTCCTGAAGAAACAAATCCCTGCCATCACCTGCATTCAGT
CACAGTGGAGAGGATACAAGCAGAAGAAGGCATATCAAGATCGGTTAGCTTACCTGCGCTCCCACAAAGATGAAGTTGTAAAGATTCAGTCCCT
GGCAAGGATGCACCAAGCTCGAAAGCGCTATCGAGATCGCCTGCAGTACTTCCGGGACCATATAAATGACATTATCAAAATCCAGGCTTTTATT
CGGGCAAACAAAGCTCGGGATGACTACAAGACTCTCATCAATGCTGAGGATCCTCCTATGGTTGTGGTCCGAAAATTTGTCCACCTGCTGGACC
AAAGTGACCAGGATTTTCAGGAGGAGCTTGACCTTATGAAGATGCGGGAAGAGGTTATCACCCTCATTCGTTCTAACCAGCAGCTGGAGAATGA
CCTCAATCTCATGGATATCAAAATTGGACTGCTAGTGAAAAATAAGATTACGTTGCAGGATGTGGTTTCCCACAGTAAAAAACTTACCAAAAAA
AATAAGGAACAGTTGTCTGATATGATGATGATAAATAAACAGAAGGGAGGTCTCAAGGCTTTGAGCAAGGAGAAGAGAGAGAAGTTGGAAGCTT
ACCAGCACCTGTTTTATTTATTGCAAACCAATCCCACCTATCTGGCCAAGCTCATTTTTCAGATGCCCCAGAACAAGTCCACCAAGTTCATGGA
CTCTGTAATCTTCACACTCTACAACTACGCGTCCAACCAGCGAGAGGAGTACCTGCTCCTGCGGCTCTTTAAGACAGCACTCCAAGAGGAAATC
AAGTCGAAGGTAGATCAGATTCAAGAGATTGTGACAGGAAATCCTACGGTTATTAAAATGGTTGTAAGTTTCAACCGTGGTGCCCGTGGCCAGA
ATGCCCTGAGACAGATCTTGGCCCCAGTCGTGAAGGAAATTATGGATGACAAATCTCTCAACATCAAAACTGACCCTGTGGATATTTACAAATC
TTGGGTTAATCAGATGGAGTCTCAGACAGGAGAGGCAAGCAAACTGCCCTATGATGTGACCCCTGAGCAGGCGCTAGCTCATGAAGAAGTGAAG
ACACGGCTAGACAGCTCCATCAGGAACATGCGGGCTGTGACAGACAAGTTTCTCTCAGCCATTGTCAGCTCTGTGGACAAAATCCCTTATGGGA
TGCGCTTCATTGCCAAAGTGCTGAAGGACTCGTTGCATGAGAAGTTCCCTGATGCTGGTGAGGATGAGCTGCTGAAGATTATTGGTAACTTGCT
TTATTATCGATACATGAATCCAGCCATTGTTGCTCCTGATGCCTTTGACATCATTGACCTGTCAGCAGGAGGCCAGCTTACCACAGACCAACGC
CGAAATCTGGGCTCCATTGCAAAAATGCTTCAGCATGCTGCTTCCAATAAGATGTTTCTGGGAGATAATGCCCACTTAAGCATCATTAATGAAT
ATCTTTCCCAGTCCTACCAGAAATTCAGACGGTTTTTCCAAACTGCTTGTGATGTCCCAGAGCTTCAGGATAAATTTAATGTGGATGAGTACTC
TGATTTAGTAACCCTCACCAAACCAGTAATCTACATTTCCATTGGTGAAATCATCCACACCCACACTCTCCTGTTGGATCACCAGGATGCCATT
GCTCCGGAGCACAATGATCCAATCCACGAACTGCTGGACGACCTCGGCGAGGTGCCCACCATCGAGTCCCTGATAGGGGAAAGCTCTGGCAATT
TAAATGACCCAAATAAGGAGGCACTGGCTAAGACGGAAGTGTCTCTCACCCTGACCAACAAGTTCGACGTGCCTGGAGATGAGAATGCAGAAAT
GGATGCTCGAACCATCTTACTGAATACAAAACGTTTAATTGTGGATGTCATCCGGTTCCAGCCAGGAGAGACCTTGACTGAAATCCTAGAAACA
CCAGCCACCAGTGAACAGGAAGCAGAACATCAGAGAGCCATGCAGAGACGTGCTATCCGTGATGCCAAAACACCTGACAAGATGAAAAAGTCAA
AATCTGTAAAGGAAGACAGCAACCTCACTCTTCAAGAGAAGAAAGAGAAGATCCAGACAGGTTTAAAGAAGCTAACAGAGCTTGGAACCGTGGA
CCCAAAGAACAAATACCAGGAACTGATCAACGACATTGCCAGGGATATTCGGAATCAGCGGAGGTACCGACAGAGGAGAAAGGCCGAACTAGTG
AAACTGCAACAGACATACGCTGCTCTGAACTCTAAGGCCACCTTTTATGGGGAGCAGGTGGATTACTATAAAAGCTATATCAAAACCTGCTTGG
ATAACTTAGCCAGCAAGGGCAAAGTCTCCAAAAAGCCTAGGGAAATGAAAGGAAAGAAAAGCAAAAAGATTTCTCTGAAATATACAGCAGCAAG
ACTACATGAAAAAGGAGTTCTTCTGGAAATTGAGGACCTGCAAGTGAATCAGTTTAAAAATGTTATATTTGAAATCAGTCCAACAGAAGAAGTT
GGAGACTTCGAAGTGAAAGCCAAATTCATGGGAGTTCAAATGGAGACTTTTATGTTACATTATCAGGACCTGCTGCAGCTACAGTATGAAGGAG
TTGCAGTCATGAAATTATTTGATAGAGCTAAAGTAAATGTCAACCTCCTGATCTTCCTTCTCAACAAAAAGTTCTACGGGAAGTAA

TABLE 4
MOUSE NOMENCLATURE
ICSGNM Zfp29
Celera mCG15309
HUMAN NOMENCLATURE
HGNC N/A
Celera hCG27579
MOUSE SEQUENCE—GENOMIC
GATCCTTCAATCGCCACCTGCCTCCCTCCCCTGTAGTGTGGGAGTTACAGGCAAGCATGGCCATGCCCCACTTTTTACGAATGCTGGGSATTTGAACC
CAGGTCCTCATGCTTGCACAGAAAATGCTCTTACCTACTGAGCCATCTCCATAATCACCTCAATTTTCTTTTCTTTTAAAAATATTTTTATTTTATTT
TTGTTGGGTGTTTTGTCTAAGTATATGCCTGTGTTCCCACAGAGGCCAGCAGAGGGCATCATAGCCTCTGGAACTGACATTATGGACAATTATGAGCT
ACCAAGTGGGCACTAGGAATCAAACCTAGGTCCTTAGGAAGAGGACCTTGGAACAGCTCTTAACTCCCCAGCCATCTCTGGAGTCCCCCACTTAACTG
TGAACAGCAGTTCTGCAAATCAAACCAAGACCTCACCCATACTAGGCGACCACTCCAGTCCTTAGCTGTATCTCTCACCCACTTATGACCTTTCATGC
TACACAAGTATTTTCATTTTATATATTTTTATTTTTCTTATTTGTTTGGCTTAGTAGACGTGTTATCACACCTGGTCATGATCTGTTTCTACCCCACT
CCCGTTTTTCATGCATGTGCTGTGGTATGCATGTGTGTATACATGTATATATGCATGTGTATATACATGTATGTATGCATGTTCATATACATGTGTGT
ATGCATGTGTGCATACATGTGTATGCATGTCTGTATGATGTGTGTATACATGTGTGTATGCATGTGCGTATACATGTGCGCATACATGTGTGCATGCA
TGTGTGTATACATGTGTGTATGCATATGTGCATACATGTTTGTATACATGTCTGTATGCATGTGTGTATACATGTGTGTATACACGTGTGTATGCATG
TGTGTATGCATGTGTGTATGCATGTGTGTATGCATGTGTGTATGCATGTGTGTATGCATGTGTGTATACATGTGTGTATGCATGTGTGTATGCATGTG
TGTATACATGACTTTTCCTGTCTGAGAGTGCACTTGTGTGTGGATATACATGCATGTGTGGACCAGAGCACGTGGAGGGCCGAGGCTGATCTTGAGAA
TTACCTTCCATTGCTTTCCCACTTTATCCAGGGTCTCTCAATCAAACCCAGAGCTCACTGATATGACTAATCTTACTAAGGAGCTTCCTCTGGAGAGT
GAGCTCCCATCTCCACTTTCCAAGGCTGACATAGGAGGCAGGCCATCATGCATACCTGGCATTTACTCGTTTCTGGGCATCCAAACTCTAGCGCTCAC
GCTTGTAAAGCAAGTGCTTAACCTGAGCCATCATGCGATCTGCTCTAATTTTTTAAGACAGGTCTTGCTTTGTATTCCTTGCTAGCCTGGAACTCTGT
GTAGCCCACACTGGCCTTGAACACTTGCCCTTTTTTAAAATTTATTTTTATTATTTTATGTGTATGATTATTTTGCTTGCCTGCATGTGAGAGCACTG
TGTGTGTGCCTGGTACCCGCTGAGTTCAAAATGTCCTTGTATGCCCTGAGACTGGAGTTACAGACAAGTACTCTTAACTTTGCAGTCACCTCTCCAGC
CCTTCTTTTGTGTTTTGATAAATGAGGTTAATTAGTGAAGAAATGGTGCATTCAAACTGTTACAGTTTCTTCTAGGCTCCACACCATGGTTACCTGGC
AACAGCTAGGTATGCCTGACTCACTATAGAAGGGACTGCTTGCCTGTCTTCTCTTACTCTTTTTTTTTTTTTTTTTTTTTTTTTTTGAGACAGGGTTT
CTCTGTATAGCTCTGGCTGTCCTGGAACTCACTTTGTAGACCAGGTTCGCCTCGAACTCAAAAATCCGTTTGCCTCTGCCTTCTGAGTGCTGGGAATA
AAGGTGTGCGCCACCACGCGCCCGGCTTCTCTCTTACTCTTTTACTCTCTAACTCTCCTCCCTTTCTGCCCCTTCTCTCCCCATTCCCCTCCCCACAT
CTCTCCACGGGTTAATGGTCAGCCTCTCTCTCTCTCTCTCTCTCTCTCTCTCTCTCTCTCTCTCTCTCTCTCTCTGCCTTTCTCTGCCTCTATTACCC
CCTAACTCCCCTCCCCATGCCCTAAATGAACTCTAGTTTATACTATACCTCGTCCTGTGCCTGAGCATGGGCCCACAGAGGCACCCCCTCACCTCATC
ATACCACACCACCTCCAAACATATCCTTGGCCTTTCTTTCTTTTTTATAAAACAGAAAACAAACGTTTTTGGGGGTGATGTGGAAATCCAATTCAGAA
TCTATAAAAAGACAGGATGAACGAAATTGACTGGATAAAAATTCAGATTTGGGGCTGGAGAGGTGGCTCGGTGATTAAGAGCACACACTTACTCTTGC
AGAGGAGCAAAGTTTGACTCTTAGCACTCACGTTGGGCAGTTAATAACCTTCTCTAATTCCAGTTCCACAGGATCCAATACCTCTGGCCTCTGTGGGC
ATCCAAATTCAAATGCAAATACCCACACAGAAACACATAATTAAAAATAAAATAAACCGTGATGGAGAGAGAGCTCAGCAGTTAAGAGCACTGACTGC
TCTTCCAGAGGTCCTGAGTTCAAGTCCCAGCAACCACATGGTGGCTTACAACCATCTTTAATGGGATCTCATGCCCTCTTCTGGTGTGTCTGAAGACA
GCTATAATGTACTTATAAATAAAATATATAAGTCTTTTCTTAAAAAATTAAAAAATAAAATGAACCTTAAAAAAAAAACCAAAACCCAGATGTGGGGC
TGAAGAGATGGCTTGGTGGTTAAGAGCATTGGCTGCTCTTTCAGAGGACCCTGGTACAACCTCCCGCACCCACATGGTAGTTCACAACTTTTATAACT
CCAGTCAGGACATCCTACACCCTCAAACCAGTGCACATAAAATAAAATAAAATAAAATAAAATAAAATAAAAAATTTAAAGCCAAATATCATTTTAAG
TCAGAAGAAATGAATTTATTTTGCTGTGTGTATGTTTGTGTGTGTGTGTGTGTGCGCGCGTGCGCGCGCGTGCGAGCCAGAGTTACAGGGAAGTTGTA
AGGCACCCATACGGGTGCTGGGATCTGAAACTCGGAATCCTTTGAATGAGCAGGAAGTGCTATTTACCACTAAACTATCTCTCCAACATTCAAATTCT
TTCATTTGATAGACTACGAGAAAGCAAACGACAAAATGAGAAAACAACAGTTCCTGGGTCAGCGAGATGGCTCAGTCGGTAAAGGTGCATTTTATCGT
AGACCACGGAGGATATTCTTTATCACTTCTCCCTTAGATTTCCTACCTGAGTGTCCAGGTAGTTACTCTCTTGTTTTTATTTAGTATGGATCCCATGG
AACCAGGCCCACAAACTGTTGTGAGCTGTAATGTAGGTGTTGGGGTCTTCTGCAAGAGCAGCAACTGCTCTTAACTCTTACGCGCGCTCAACTGGCCA
GGAAGAACGACGCTGCAACAGGATCCTTCTGCACACGTTTATTGGGAGAGCTTGATTGTAGAGGCGAAAAGACCCTGAGCCCAGAACTGGTGCTGCTT
ACATAGGCCTAGGAGAGGTGTGTCTCACATCTGATTGGTTAACTTGTCTTGGCAAAAGAACCTTCACTGCCTATGTATGTGTAGTGGCCAGCAGTAGC
CAACTGCCACTCTGCAACTGCCACTCTGCAACTGCCACTCTGCAACGGCTTCCCACACTTAACCACTGAACCATCTCTCCAGCCCCCAAGTCAAGGGT
TTTAACTAGAGCGAGTAAGGGAGTAAAAGTTTGTATGCTCTTTGGGAAGAGAGCAATCCGTACAGAGTAGGAATGGCTGTGTCGGGGACAGAAGTGGG
TTTAGATTTATCCTTTATCTGTTATCCTCTCCCTGTCCCTCCATTAAGGCGGGCACTAAAACAAAGTGGCACACTTTTCTTGGAAGCTCATTTCACCC
AGTCTTGGGGCTTAACTGCCCTCAAAGCTGAAAAGTTCACTTGGTGAAGGGGTAGCAGGCACTCCATGCATACTCTTATCTAGAGATACCCTGAAGCC
AGTTGAAGCTGAGCCAACTAGTAGACCACTACCATCTATTGCTGTTCTTTACATCCTGTTTTGGGGACCTTGAGATGACCCATCTGGAAGGACCACTA
TTGTCCTTTGTGTCCTGTTTTAGGGTGCCTGGGTGGTCATTCCCTCCCAGAGCTAACCTCTTGCCTAACTGTAGGGCAGGTTCCAAGGAGCCGACTAT
AGACTAGGACTTACAAACCAGAAAGAGCGGGGCGGGGCTTTCGTGAAGACGCAAGAATACCACGTGTGAAAACAAAGGGGAGTGCAACCCCGGAGTGC
AAATGCCTGTGCGTAGCTGCAAATTTCCAGGAGAGGTGCGAACTTGGGCCAAGAGGAACTTTGATTGCGCAGATTTCTTCTCTGCTTAGAGTCAGCTT
TTGGCTCTCGAGGGCTCATTTGCCACTCAATTCTCAGCTGAAAAAGACTGATAAATTCACCCAGCTTTCATTTTTTATAGATAAATACACGCGAGCTT
TAGAGAATTGGCACTATCTGTCTCAGGTGAAAGCTTTGGGCAGTACTAGAAGACAGACAGCTAGTCCTGGGTCGTCCTCTGCCTCTGATCCGGAGGCA
TTACTTACGGCTATCAAACCCTCTGTTCGCTCAGGTACAAACCACCCCCCCCCCCGTCTCTGGGTAAACTACATTTCCCCCAATGCATCGGGTGGAAC
TTCCCTCCCAACGCCCATCTGGACGCAGTTTTCACCAATAGTGGAGCAGAATTTCAGGAACTGTTGTGGACAGCCAATGAGAGTGGCCATGGGCGGGC
CTCTCCCGTCCATTGTTCTCTGTGCCCCTTGGGCTTGAGCTGAGGTGAATCCAGAGGGGCCGGGCCGGGCCGGGCCAGACCGTGGCGTCCTTTTGCGC
TCAGAGAGATAGCGGGAACAGGACCTGGTCCCTGGAGAGGCGAGCGGGAGAGGCAGGTCAGGAGCCGCGCGCCCCGCGGGGAACAGCCAGAGAGACAG
CGAGTAACGGGCCTGGAGCCAGCTCAGGGCAGTTTGGGGGAGGGGCGTTCGGTGTCCGCGCCCGACGGGCTGGACGCGCAGCTCWGCGTCCTGGTTCT
TCGGAGCCCTCGAGGCTCCGCTCACACCAGAGCGCTGGGTACGCGCAGCCTAGGAACGCAGGCTTTACACTGGGAAGGCATCCTCGACGACATCCCAC
CGAGCCGCCTCCTTGACCAGGTGGGGAAACTGAGGTTCTGAGGGGGCGTGTCGGAGCCAGGCTTAGCTAATAAGCTGTCTAGGTTCAGTGTTCCCGGG
ATGTCCAGGAAGAATTACGATGTATTTGTGTTTCACGTAGTCATTTTGGACAAATTGACATTGGGAGCTTCAGTTTTCTTTCTGTTGTTGCTGTTCTG
TTTTAACTTTTGCTATTTTCATGGGGGAGGGCATGTTATCTGATTCTATGGTGTAATGATTTTTTAAAAATAAATATTCATTGCTTTCATAATACTTA
TATAGTTTTTAATTCCACTGCTTTGAATATTTTGTAGAGCTACTACTCAGATCTAGGTGTGTGTCCCCCAACACCCCCACCCCCCGAATGATCCCCAT
GTGTGTTCATTTAAAAAGCAATTGGGCAGGCAGTAATCTCAGCACTCAGGAAGCAGGACAGGAACGTCAGGAGTTCGAGGCCAGCGTGGGCTACATAA
TGAGATCTTGCTTCAGAACCAAAAAAATAAACAGAAATCTCTGTACTGAAAATGTAGGACCGTTCTCGTTATTATTCCCAAACAGTACAGCATACCAA
CTGTTTACATATTATTTACACTTAAGTATATGTTGTCTAGAGATGATTGAAGGTATTTGGTACACCATTTTATATAGGGGCCTTGAGGAGTCCTGGAT
TCTGGGATCTGGCAGATTATCTCAGGGCAACCTCCTTTGAAATATAGCACTAATGACTTGTAGTGAAGCTGGGGGCACCTTGACATGCGATCCATTAG
TAATCACTATGATGTTCCATATGATAAGGATGAGTGTTATCTATTCATTAAGTGGTCACTGAGGTTGAATGACTAACCTCTTTGTTAGGTAACAGTCT
CTGAAGGGAGTCTCAACTGACTTAGGGTACGATATGTCACTTCTCTTTCTCTCAGCCAACATCACTGCATCCCACTACTCTTCCTTTGGTCTTCTGAA
AAGAGCAATGCTGTGTATGTGTGTGGTAGTTGTTATTATTATTATTATTATTATTATTATTATTATTATTATTATTATTATTGGGTTGCTATATCCCC
TTGTTGTCTTTAAATATCTACTAGGTGTTTCTACCCCACTTCAGACCATATTGTTCCCAGATAAAAGACACAGAAACCTGTAGATTCATAATAAGCTT
AAAAGCATTAAAGTTGCGCTATTTTGTCTACCTCCCAAGGTATCACTTGCCATGCTCTGCCTGGGCCGCTTCTACTCCATCAGGCAAGCCCCTATAGC
CATGGGCTCATGAACTACCTCCCCCATGGCCACTTCCTTCTTTCTTTTTCTCTCTTCATGGTCTCTACCTCAGATCCCAAGCCTGGGAACCTTTGCTC
CACCCCCTGCCTTCTGCCCAGTCATCTCTATTGGCTGGGATAAGTTGGCGGGTGGGGCAAGGTTTACACAGCATCATTTGGTGTATATGAGACCTTCT
AGTTGCGTGCAACCAGATCTTGGGGGCCAGTATTTAGATTTGAAATAGTGATATCAGACCAATGTTGTCTGTCTGTGATATTTCTTCTTTTTTGGGGG
GGGGGGGGTGGGTGTTTCAAGACAGGGTTTCTCTGTATAGCCCTGGCTGTCCTGGAACTCACTTTGTAGACCAGGCTGGCCTTGAACTCAGAAATCCG
CCTCCCTCTGCCTCCCGAGTGCTGCGATTAAAGGTGTGCGCCACCACGATGGGCTGTCATATTCCTTTTGCCATAGAGAGTCACTGCTTTTACAAGTA
AGGCAACAAGCCTCTGTTGGTTTTAAATATGCGGAGGCTGCTATGAGATGGCTGTAGCAGTTTCCTTACTGATTCTGGTTCTCTGCCTTGTTTCTCAG
CAGTGTTTCTTGTTGAGATTGGAGGAAAGACGGCCTTCTCAGAGAGCCTGACTGGAGACAGGTGTTAGGCTTGAAGCCTTCGTGACCATCCAGGAAGT
TGGACAATGGCAGCCGAAGTGCCAGCAGTGAGCACTCCCCTCAGCCCTTTGGTTCAGGTACCTCAAGAAGAAGATGAACAGGCAGAGGTCACCACTAT
GATCCTGGAGGATGACGCGTGGGTGCAGGAAGCAGTGCTGCAGGAGGATGGCCCTGAGTCTGAGCCCTTTCCCCAGAGTGCTGAAAAAGGCAGCCCCC
AGGACGAGGACGCAGCCGAGCGACCCCAGGGTGCTCTTGTCCGATTTCGGGAGCTCTGTCGGCGCTGGCTGAGGCCAGAGCTGCACACTAAGGAGCAG
ATGCTAACTGTGCTGCCAAGAGAAATTCAGGCCTGGCTGCAAGAACATCGGCCTGAGAGCAGTGAGGAGGAAGTGGCCCTGGTGGAAGACCTGACCCA
GACTTTTCGGCACAGTGCTAAGACAGAACCACACAGGGAGAGCGTGGGAGCCTTCGGAGGTTGGAGTAGTGTCAGGGTTTTCTTGCTGTTGCTGGTGT
TGGTGGTTGTCGTGTGTGTGTGTGTGTGTGTGTGTGTGTGTGTGTCTACAGAGAATGGTCTTTGAACAGACTAGGAGATGGCGCTTGTCTCTTATGCT
CCCTGCAGCACTCAGCAAGATCCTAGCTCTTTTAGATCCCTCACCAATGTGCAGTGGTAGCTCCAGAATTTCAGTCTGAGGACTCGGAGGGCTATTCT
TGGAGCTCCGTTTGCATACGAAACACAGCTTTCACTTGTTTATTCCAGGGATTGGAAAGTTTCCTGAGATGAGAGATAAAGGTCAATGGTAAATAAAA
TGCCTGCCTAGCACGCACAGGAGGTTATCGGAAGCTATTTTTGTGTTTGTGATTTTTAATTTTCCTTGCTATAGAGAAAAAGTGTTTCTTCCTCCTCC
CCCTCCTCTTCTTCCTCCTCTTCCTCCTTCTCTTCTTCCTCCCCCTCCTCTTCTCCTTCTTCTTCTTCTTCTCTTCTGTCTTCTGTCTTCTGTCTTCT
TGATCATGTCTCATTATCTAGCTCTGGCTGGCCTAAACTCACTGTGTAGACCAGGCTGACGTAAAACCCACAGAGATCTGCCTGCCTGCCTCTGCCTC
AAGAATAAGACATTCTTCCCTTAAGGGAAACAGAAAATTTCTGTTGTTTTGCAGGTATGAGAACAAAAAAGACCTCCATAACCCACCCTTTGGTTCCT
TCAGTTGGTTGTAATCATTCAAATAGTTCCAGCAATTGGAATAAAAGCATGCCACTTTTTTCAAAGTGGTCATTGTATGAAACCATGATAAACAATAA
GTAAATTGTCTGGGCTTTCTGTGATATGATGTCATCTTATATCCTTTAAAGAGTAATTGGCCAGGCTAGGAAGGGCACACCTATTATCCTAGCACTTA
GAACCCAGGCTAGTATCTACTTCAGTTCTTCCTTTCCCTAGGATAACTTCAGGACCCTAAGATGTGGCTGCTTTGTGTTGATGAAGTACTTCAGGAAT
ACAGAAGGGCATGTAGAGAGGACAGGCCAGACCAGAGAAGGCTTCGTAGAAGACATGGACCATGAGTAGGAGCTTGCAGACTGAAGTAGAAATTGGAA
AGTCACAGGGCGAGGAGACAGCTGTGACTGCTGACAGGAGCTGTAGGTAGCCAAGCCTCTGAAGTGGGGGCAGCAGCAGAGGAGGAGGTTTCTTCAGA
TTCAGAGCCCGTGAGGCAGGCTTGTGTTTTCACGAAGTGGTGGCAGTTTGGGGCGAGAAAGGATGGAGGTTTGAATGAAACGCCATTGGCCTCAGCAG
GCTCTTGATCTAGGGCCTGTGGGGCAGGAGAGGAAGCTGAGCTTGGCAACAGGATGTAGGTTTGATTAGGGGACAGACGCTAGAGGTTGGGGAGCAGT
CAGGAAGCAGCCTGGCCATGGTGTGTGCAGGGACAAGAGTCTGGCCGTGGGACGATGGGACGTGAGAGAAAGAACAGCGTCGTGGGAGTGCTTTCACC
AAGCCCTCAGTCTGCCGGAAGATGAGTGGACTATTGTCCTTAGAGGAAGAGTGACCCACTCTAAGATCACGCAGCCAGTATGAGGCAAAGCCAAGACT
CTTGTCCTTAAGCATGCCATTGCAGGAGGGGTCCTAATGATTTCTAGCAAGGTGCTGGAAGGAGGAGATCCCAGCAGGGATTGGGGCAGAGCCTCTGT
ACTGTCCACTCTGGACGACTTAGTCGATTAGAGAGGGAGATCATGAGGAGCGGGTAGTTCAGGGCAGAAGGTGTGACTCCAGCCAAGCACTGAGAGAT
ATGGAGAAGTCTGTCCTCAGAAGAAAGGTCTGGGCTTAGGGGACTGACTGAAACTAGCCCCTCGCTGGGCACCTTATATGGCACCCACACCAAGAATC
TTGTGAGATGAGTGGGACCGTCACTGGGCAGAGGAAGCGCAACAGCTTCACACAGCTGTCATTCGGTAGAGCCAGGATTCAAACTCGGGTCTGTTTGC
TTCAGCCTGAGTTTATAGGCAGGACGTTTTATTTGAAGTGAAGTATTTTACTTTCTTTGACTTTGACAGATAACTTCCTTAGGGGTGTGCTTGAGATC
AAGCCTAGGAAGTGGGTCATGTGAGGAAGGGCTGTGTCACTGAGCCTCACCTACAGCCCAGGAACTTAATTAACCTACCTTTGAATTGGGAGCGTGTC
TCTTCCCAGCTGGCCTAAAGCTGGTATAGTCAAGGATAAACTTGAACTTAAAAATATTTCTTTTACTTAGTTTTATGTGTGTGGGTATTTTGCCTGCA
TGCATGTATGTGCACTGTGCATGTGCCTGGTGCCCTCAGAAGGAGGCATCAGATCTCCCAGAACTGTATTTATGGACAACACTCTGAGCTGACAAGGA
GGTGCTGGGAATCGAACCTTGGTCCTCTGGGAGAGCAGCCAGTGTGCCGAGCTGCTAAGCCATTTACAGCCCCACCTTGAACTTTTGATGGGCCCACT
TCACCGCATAGGATTGTAGATGTGTGCTGGCGTTCCTACTTCTTGTGGTGCAGAGGCACCAAACCCAGGCCTGCTTGCCTCTTTGACAAGCAGGGTAC
CAACCTAACTACATCTTTAGTCAGTTTGTTTTCAGACACATTGTATCCTAAATTCGCCTCGGTCTTCTGGTAATCCTCCTGTTTCAGCCCCCTGAAGT
CTGGGACTATGTGATATGCTACCATATTTGACTTGATTTTATACTTTGTACAAAGAAGAAGAGGGGAAAGAAACTCTGTCATCCCGTTTCAGATCTCC
CTTGCCTGCTGATGGCTGAGGCGAGTTTATGATGACCTTCAGGGTTTCCAACTCACCCAAGAGGCTTGGGCTGACGTGTGTTTGGTGATTGTTACCTT
AGGGCAGTGCCAGGTCAGTGCTGTGGCTCAGCAGGGGTCTGAGGTGACCACAGCCCTCACCACAGCCTTGCAGTGCCTTCCTTTCTCATTGGTCATCT
GAGTTGGTGGTGGGTTCTCCCTAGAGGAATCAGGAGGCTACCCTGTGACTTCTGCCTCTAAGATTATCTCTGCTGGCCTCTGTTTGATAACACGTTGT
GCCCTTCTTCCTCATAGCTGCTCACAGGAATCTATATTTTCCTTCATTCCTTCTTAAACATCGATTACCTGAGAGCAGCCCGCCTCTATTCTCATTGT
TAAGATAAAGGCCTTAGGGTTTGTTTACACAGGAAAGGGCATTTCAAGGCCTGGTGAGTCATTGGGTAATATTTGACTGAGAGGCACCTCTGGGTGTA
TGCACTTAGTGTTCTGTGACTCCAAGTAAAACCATCCCTCCCGTATAGTTACGTGTAAAGTTCAGCTCTATTTGTCCATGAGTCCCGCAGTTGTTTTA
GCTCCATTTCAAGCCCTGAATCAGGCTTTGGGATTTCCTGACTGAATTTTTGCAGTAGTCTCTGAACTAATCTTGTAGTCTCTTTGCCAGTAATCAGA
CTCCTTCACATCGCCCAGAGCAGTCCACCTGCAGCCTCCACAGGCTTCTCCCTCCTTCTCCAACAGGACACTTGCTCGCTCTCTCTCTCTCCCTCGCT
CTCTCTCTCTCTCTCTCTGTTTTTGTTATTTTCTGGCTTTTTGTTTTGAGATGAGGCCTCATTGTTGTGTCTAGACGAGGCTGCTCTTTTTCTTTTGT
TTTGGGTTTTTGTTTGTTTGGTTGGTTGGTTTGGGGTTGTTTTTTTTTTGTTTTTGTTTTTGTTTTCCCTAAACAGGGTTACTTTGTATAGCCCTGGC
TGTCCTGGAACTCACTCTGTAGATCAGCCTGGCCTCGAACTCATCATCTTCCTGCCTTAGTCTTTTGACTCAATCTTGTTTTTCTATTTATATACTCT
TATATCTTTTAGGAACTTAACAAAAGTCCCAAAGGCCAGGGACCTAATCCACTCCTCCTAATCCTTTCTCAGTTCCTGCTAACAGTGAGCACTCAGTG
AGTATTGTTGGGATAAATGAAGGACACTTTTGCATCTACAGGTCGAGAGACCTCTACTTGGGATTTCCTGACTGAATTGTTGCAGTGGTCTCTGAAGA
CAAGGTTGGAGTTGAAAATCTGGGTTACAGAGAATTCCCTTTGGTGCAGAGGACAGGGAAACCGCTCAAGCTGATGATGAGCTGGGTCTGAGCGATGG
AGAATGAGACTGGGAGACTGAATAGTCAAAGCTCAGAATAAAAGATGGAGGAGTTGGCTCAGCAGCTAAGAATACGTACTGCCCATGCAGAGGGCCCA
GGTATGGTTTCTAGCACCCAGACAACAACTTAAAACCATCTGTGGCTCCAGTTCCAGGGGGTCTGATGCCCTCTTTTAACCTTCTGGGAAACAGGCAT
GCATGTGGTGCACGTGCGGGCAAATGCCCATAAAATAACATCTCAGGATGACATCACTGCTCCTGGGAGACAGCCTCTCAGTGACTTAGGGTTTGTAT
GAGATTCACCAGTGTCCTTGTTTGATGGCTGTTTTCAGTCCATGACAGACCTGTTAAAAGACCACTTATACAAAGCAATAGCTAACTTCTGTAATGTT
TACAGATTATTACTGAGGTAAAAAACAGAATGCTTGTGGGTTTGTTTGTTGTCTGTCTGGTTTTTCCCCCCATAAATGCTTTGGGGAATGAGATTCAG
GTTTTCTAGAAAAGTCCTGTATTTACAGTTATGAGCCCTGGCTGTTCTCCCAGAGGACCTAGGTTTAGTTTCCAGCACCTACATGGCAGTTCACAGCT
GTAATTCCACTTCCAGGGGATCTGACACTCTCACATGGACATACATGCACCTCACATGTACTCACACACACATACAAAACACCAATGCACATGAAATG
CAAATAAATTTTTAAAAATTATTTGTCTTTTGGATGGAGAGAGTGGTTAAAAGTACTTAATTGCTTTTATTGAAGACCTAGACTTGGTTCCCAGCACC
CACATCAAGGTAGCTCACAACTGCTTATAACTCCAATTCTAGTGATGGGGTGTAGTGGCACACGTCTGAAATCTTAGCATTCAGGAAGTGGAGCAGGA
CAGTTGTAAGTTAGAGACCAGCCTGAACTCCATAAGAAGTTACGGTCCCTAGTGGGAGGAAACTCAATGGCTGTGCATTTGCCTGGCACACTTGAGCA
AATCCTAACACCTGGCCCTCTCCCAAATTGTCCAAATATTTTCCATTCCAATAGTTGGCAGTTAAACTATCCAGTGTGCTTCATAACTGAAGTTAGAT
GTCATTTCTCATGGCCCTAATATTTAAGTTCCAATAATTCTTGGTATAAAATGGGAACAGGTTGCCTCTGTACTGACCTTTAGGGAAGTGAACCCAGG
CCCCAGGCAACCCCAGGCCAGTGAAGTAGCTTCATCAGGGTGGACGTGAACCCTCTCTCTCTCTTTTTTTGTTTGTTTTCTTGAGAAAGGGTTTCTTT
GTGTAACAGCCCCGGCTGTCTTAGAACTTGCTTTGTAGACCAAGCTGGCCTGGAACTCAAGAGATTCACCTGCCTCTGCCTCCTGAGTACTGGGCTTA
AAGGCATATATCATCAGGCCTGGCTTGAGCTAACCCTCCTTGATAGATACATTGACCACATGGAATTCCATCATCCCAATGCATGGTAGATATCAAAG
GGAAGGAAAGGGGAATTATTAGTCTCAAAATTGATAAATTCCTGATAATAGTCACACCTGGTAGCACACACCTATAATCTCTGTGCTGCTGAGGTTGA
GGCAGGAGGATCATGAATTCAAGGTCAGAGTGGGCTACATAGTGAGTTCCAGGCAAAGTCTTCAACCATTACTACAGGGTTTGTCCCCCTGGACCAGC
AGCATCAGAATTGTTAAAAATGTGAATCCCACTCCAGAGCCAATGAGTGAGAAACTTTAAGGGTGGGATCAGCTCTTTAGGGTCTTACAAGCCTTATA
GGTGATCTTAGCTTAAGCTCTAGTCTCCTAACCACTACACCAGGTCTCTGGTTTTTAAAGTTGGCAGGTGTTGAAATTACCTGGGGACCTAGAGTCCA
TGCCAGGGGTGCTTGCTAGAGACTGGACCCACTGATTTCCATACGCTAAGCATATGCTCTATTGAATGACACCGTAGTCCCCATGCAAAGGTTTTTGG
AGGCTCCATAGGTAGTTCTGTAATGCAGTTGTGTGGCAATGTAGCTTTTTTACTCAAAGGATGGTCCTTGGAACACAATATCAGCAGGAAGCTGAGGA
GAAATACACACTATTAATCTTACCCCAGGCCCACTGAAGCAAAATACACATTTAATTAGATCTCTGGGTGGCTCTTCTGCACCTTGAAGAATGAGAAG
CCTATGCTGGGTTAGAGAGGAACAGGGGTGTTACATTCATACCTGTTCTTGTCCCACAGATGAGCCAAAGAATCACAGCTTCACCCTCACTCACCACA
CTTCACATATTCCATAGTGCGCATGTGGAAAGCTTTCGGGAGCTGTTTCTCTTTCTACTATGTGGGTGGGTTCTGGGCAACAAAATTTGGTCATCAGG
TTTGGTAGAAGGCCTCTACCTGCTGAGTCATCTTAGTGTTCTTCTTCCTTCTTTAAGGAGAGGGTTGTGTGTAGCTGAGGTCTTGAAATTCTATGTCT
ATAGCCAAGGATAACCTTGGGTTCCTGATCTTTTGCCTCCCTCTTGAGTGCTGGTCTTTCAGGCATGAGCCACCGTGCCAGGTTGATAGGGTTCTAGA
GATTAAACCCAGAGCTTTGTGCATACTAGGTAAGCATTCTACCCCACTATGCTACCTGGCCAGATCATATTCATTATTTATTTATTTATTTATTTCAT
GTATGTTGTATACTGTCACTGAAGAGGGCATTGGATCCCCATTGCAGATGGTTGCGAGCCACCATGTAGTTGCTGGGAATTGAACTCTGAACCTCTAG
AAGAGCAGTCAGCCCTCCTAACTGCTGAGCCATCTCTCCCGCCCTCATATTCATTTTGTTAACATGAGAGGTAGCACTGTTGGACAGGTAGACTGTGT
TAAAACTCCACTTTTTATGTTTAAAGAGGTGGGTCAGCTGTTAAGAGGGCCTGAATTTGGTTCAGCACCACTATCAGGTGGCTCCCAAATGTCTGTGC
CTCCGATTCCAAGAGAATCCTGAGCCTCCAGGCTTCTAGGGTACCTGCACTCACATTCCCACACAATATACATAATTAAAAATTATAAAATTATAACA
AGATAAAAAACTTCAGGTCTTAGCTTGAAATCAAAAGCACATTCATTCAGTCAAATCTTTGAATATATACCACATAGAGCTCTTAAGCAGAACTCACC
CTACACTGTAAAATGGACAGTAGTCATGAAACTGATAACTGAATTCATTGCTGTTTTCCAGCAGCTGGGACTGAGGCAGAGCATCAATCGCTCCTGGT
CGCTGCAGCCCATGTAGGTTCCTTCCACCTTGCCTGGATGGCTTTGAGTGGTGGGCGGGCTCCTGTGAGAATGGAGAGGACCTGGTGTGGGCATCAGA
CCAGTATCCTGATCACTGTTTTAGCCCAGTGGCTGGAACCACAGTGTTGAGACACAGTTGTGTGTCATAGACAATTGGCAGGACTGAGCCAACTCAAG
CTTCTGTCTATAAGTAAACTATAGTAACCATCCCTGACTACAGGTGATTACATCTAAGACACACTCCACTCTAAACACCCTCCGTGTAGCACTTCCCA
CATACATTCACAACCATGATAATGTTTAATTTATGAATTATACATTATAATAACTATAAGAGATTGACAGTAAAGGCTACAGTAGAAAAATTATAACA
ATATGTTATACTGCAAACTTATTTAACCTGGCAAATACCTTTATTCCAACACTGGGGAGGCAGAGGCAGGCAGACCTCTGTGAGTTCAAGGCCACCCT
GGTCTACATAATGAGTTCCAGGATAGCCAGGGCTATGTAGAAAAAAAAAATTAGAATCTGTGAGTTCTGGAATTTTTTTTGTTATTTTGTTTTTTTTT
TTTTTGTTTTTTTGTTTTTTCGAGACAGGGTTTCTCTGTATAGCCCTGGCTGTCCTGGAGCTCACTTTGTAGACCAGGCTGGCCTCGAACTCAGAAAT
CTGCCTGCCTCTGCCTCCTGAGTGCTGGGATTTAAGGCGTGTGCCACCACGCCCGGCTGAGTTCTGGAACTTTTATGTACTGTTTTTTGCACTTTAGT
TGACTCAGGGTAACTGAAACTTTAGGAACGGAAGCAGGAGATAGGGGGGACTATTCCAAAGCACATTGGTTTATCTTCATCTTTAACATCAGTTTCCT
TCGCTCATCCTGAAATGCTTTTTGTTGTAGGAGAGAAAGGGCCCACAGCTAGGCTGGAAATGCACAGGTCCTCAGGAGAGCCATAGGACCATGCTGTA
CACCTGAGCCTAAAGAATGTGTTAAGGGGGTGGCTGGCGAGATGGCTCAGTGGGTAAGAGCACTGACTGCTCTTCAACCACATGATGGCTCACAACCA
CCCATAATGGCATCTGACACCCTCTTCTGGTGCGTCTGAAGACAGCTACAGTGTACCTATTTATAATAATAAATAAATCTTTGGGCTGGAGCAAACAG
GGACTGAGCAAGCAGGGCCAACCGGAGTGAACGGGACTGACCAGGAGCAAGCAGAGGTCCTAAAATTCAATTCCCAACAACCACATGAAGGCTCACAC
CGCATGAAGCTCACAACCATCTGTACATAAGTAAATTAATTAATAACTGTTAGCTCTTAAGTGTAACAGTTCAACTGCTGAGTGGACAGTTTTTGGAT
AGTTTTGAGAAAAACTAAAATTGAGGCCAGATATTGTGGCATACTCCCTTAATCCCAACACTCCAGAGACAGAGGCACATAGATCTCTGAGTTCAGGT
CCAGACTGGTCTCCATCGAGTTCCGGGACAGCCTGGTCTACAGAGTGAGTTCCAGGACAGCCAGGGCTACACAGAGAAATCCTGCTCCGTTAATTATT
AATGTGACTATATATGTATCTATGTATACATACACACATATACTTATATGCACATATATTTATATGTAGGAAAATAATGTCAGCACAGTTTATCTTTA
ACCAGCCAGTTCCCCAACGACACCAAGAGACTATGATCTACTGACTTTTTTTTCTTTTTTTCCTTTCTCTTTCTCTCTTTCCTTTTTTTTTTTGTTTG
TTTTTGTTTTTTTTTTTTTTGTTTTTTGAGTCAGGGTTTCTCTGTGTAGCCCTGACTGTCCTGGAACTCACTTTGTAGACCAGGCTGGCCTCGAACTC
AGAAATCCCCCTGCCTCTGCCTCCCAAATGCTGGGATTAAAGGCGTGCGCCACCACTGCCCGGCCTATGCACAGCTTAAAAGCACAGTATTGAGCAGC
CATTCCTATAAGGTAGTCTGGATGTCTTCCTGCCAAAGTCCCCACAACCCTTGCATTTGGGCGTTCTCGCTCCAGCAGAGCCCTTGGTACCTCTTGGA
CCCCTCCTGTGGTTGGTCTTCTACTTCCTCTTTCCTTCCCCTCCCCTAGCTGGGATCAGGACTCTCCCTCCACCCAGCACTGCTGGCCAGCGTTTATT
GACAAGGCAGAGAACTAATGCTGAGCACTGTTTATACAAACAGGAGGCCGGAGTTTCCCAACACAAGCATTACAATGCTGCCCTGTCCCCATTGAAAT
GAAGTAGTGAGGTAGAAAAACCAGCATTTGGATAACAAGCGTAAACTTTACACAGTGTACAAAATCATGCTGTCAGGATTGCTCAGAAAAACAAAAAC
AAAACGACAATAACAACAAAGGGCAGGCATGTGTATGTAAATACAATTCCAGCCCTGAGGAAGGAGCCCTGGGGCTTGCTGGCCAGCCAGCCTAGCCT
AGTTGGACAGACCTAGGCCAATGAGAAATGGGATCTCAAATGGTGCCTAAGGAAGAGCTGCACCAGTTTGCTTGCCCTCTTGATTCCACATCACACAA
TGTTCTTGGTCTGAACTCTTGGCTCTGTGATTCTAATCAAGCAAGGTAGAAAGGGCTGCAGAAATGACTAGGGGGTTAAGAGCACTTTCTGTTCTTCC
CAGAGGACTCAAGTTGAGTACCCATGACCACCTGTAAGTTCAACTCCAGTGGATCCGATGCCCTCTTCAGGCCACTGAGGGCATCTACACAAGTGTGA
TATACACACAGCAGAGTAAAAATAGACACATTTTTATATACCCCTGAATATCAGGAACAGAGACTAAGAGGCTCACAAGGTTGGGTTTGCTTGGTGTT
GAGGTCAGGAAGAAAACGCCCTCTGCAGGCTCATAGCAGGCAACATGTGGCATAAACAGCATTGTAGACTGCTTCCTGGCAACATCCATGTTCCTGAC
AGTCATTAGTTCCTAAGGTATGTATTTCTTTGGGTGTACGTAGTGTCTGTAGGCCATTACAGCAGCACATGGTTCAGTAAACAGTCTCACAGGGCACA
GTGTAGTCCAGGTTTGTGGTTGTTCCTCATCAAGCTTCATACTCATTTTATGTGTATGGCTGTTTTGTCTGTATGAGTATCTGTACCAAGTGTGTGCC
TACTGCCACAGAGGCCAGCATAGGTATCATCTGAGTTTCAGGAAGTTAGGAGCCACTGTGTGGGTGCTGGGAATCGAAAATTTAAAAGAAAACCCCAT
AACATCTGTGCCCCACACACATGTCTTACCCACAGAAGCGTAAGAGGGTATCATGTTCCCAGCACTGGAGATACAGTTGCTAGTCACCATGGGATTGC
TGGGAATTGAACCACAGACCATTGAAAGAGCAGCCAGTGTTCTTAACCACTGAACCATCCAGCCCAATATCCATCTTTTTTTTTTTTTTTTTGAGACA
GGACCTCAGTGGCTTGGAGCTTGCCATGTGGGATAGTCTGGCTGGCCAGTAACTCCTAGGGATCTGCCCGTCTCTCCTTCCTCAGCACTGACCTTACA
AGCACACACCTCCACAGCTAGGTTTTATGTGGGTTCTGGGAGTCAAGCCCAGCTCCTGGTGCTGCCAAGGAAGTACATTACCAACTAAGCCATCTCCC
AGTCCTCTTCATTTTCATTTAAACATGTGTGTGTGTGTGTGTGTGTGTGTGTGTGTGTGTGTGTGAGAGAGAGAGAGAGAGAGAGAGAGCGCACATGT
GTGGAGAGCAGAGGACAACTTGCTCGAGCTGGTTCTCTCAGGCTATCAGGCTTGGTGGCAAGCACCCCCCATCAATGCTTTATCTTTGTTTTTCTTCC
TTTCAGATTTTGAGATACAGAGCGAGAATGGGGAGAACTCAAATGAAGACATGTTTGAGGGTGTGGAGTCACATGGGATGTTCTTGAACATCTCTGGA
GGGGAAGGTGGTCAGCAGTCTGATGGGGACAGTGACTTTGAGAGAGACTGTGGCTCTGGAGGCGCCCAGGGACATGCCCCGGGTGAGGACCCCAGGGT
CGTGCCATCGGAAGGAAGCGAAGTTGGCCAGCTAATACGCCTTCAGGGCACCTACCTGGGTGAGAAGCCGTATGAATGTCCCCAGTGTGGGAAAACTT
TTAGCCGGAAATCCCACCTTATCACCCATGAGCGGACCCACACAGGAGAAAAATACTACAAATGTGATGAATGTGGGAAGAGCTTTAGTGACGGCTCG
AACTTTAGTAGACACCAAACGACTCACACTGGAGAGAAGCCCTACAAATGCAGGGACTGCGGGAAGAGCTTTAGCCGGAGTGCGAACCTTATCACGCA
CCAGAGGATCCACACCGGCGAGAAGCCTTTCCAGTGTCCCGAGTGTGGCAAGAGTTTCAGCAGGAGCCCCAACCTCATCGCCCATCAGCGCACGCACA
CAGGGGAAAAGCCGTACTCGTGCCCCGAGTGTGGCAAGAGCTTTGGCAACCGGTCCAGCCTTAATACTCACCAGGGCATTCACACCGGAGAAAAACCC
TACGCGTGCAAGGAATGCGGCGAAAGCTTCAGTTACAACTCCAACCTGATCCGACACCAGAGAATCCACACGGGAGAGAAACCATACAAATGCACCGA
GTGCGGCCAGAAGTTCAGCCAGAGCTCCGCGCTCATTACGCACCGGAGAACGCACACCGGGGAGAAGCCCTATCAGTGCGGCGAGTGCGGCAAGAACT
TCAGCCGCAGCTCCAACCTGGCCACTCACCGGCGCACCCACCTGGTGCAGAAGCCGTACAAGTGCGGGCTGTGCGGCAAGAGCTTCAGCCAGAGCTCC
AGCCTGATCGCGCACCAGGGCACGCACACCGGCCAGAAGCCCTACGAGTGCCTCACGTGCGGCGAGAGCTTCACCTGGAGCTCCAACCTCATCAAGCA
CCAGCGGACGCACACCGGCGAGAAGCCCTACAGATGCGGCGACTGTGGGAAGGGCTTCAGCCAGCGCTCGCAGCTCGTGGTGCACCAGCGGACGCACA
CCGGCGAGAAGCCCTACAAGTGCCTCCTGTGTGGCAAGAGCTTCAGCCGGGGCTCCATTCTGGTGATGCACCAGCGAGCGCACTTGGGAGACAAGCCT
TACAGGTGCCCGGAGTGCGGGAAGGGCTTCAGCTGGAACTCCGTTCTCATCATCCACCAGCGCATCCACACGGGAGAGAAGCCCTACAGATGCCCGGA
GTGCGGCAAAGGCTTCAGCAACAGCTCCAACTTCATCACACACCAGAGGACGCACCTGAAAGAGAAGCTTTACTGAAGTGGCAGAAAAGAGAAGGAAG
TGCTGAGCTGACTCTGCAGGGAGATTGTATCAGGTCAGGATGATAGATCTCCCAGTCGGAGTAATCTGTAGGAAGTCGGAGCCCTTCAGAACACAGTC
TGAGGAAGTATGGCCTGAGACTGATGTCCCGCTGTCTCTTCCATTGGTTAGAGGGACAGTGACTGCCAGGAAGAGTGTCAGCTTAGATGTGTGTGCCT
GTGTGTGGACCACACTTGGACACACACAGTTTTATGTTTGGAACTCGAGGCCTCTGACCTCCAGCAGTCCCATCAGAGTGAAACCGTCGTGCGTGCTG
TGCACTGTGTACACAATCACTCTTGTACTTGTTTGTTCCCTGCATAAGTACCTGCAAACACCCAACACACACACACAGAGTGTATGTATCACATAGAA
CGCAGGCTGGCCTTGAACTTATGATGTAGCTGAGGATGACCTTGAACTTCTGATCCTCCTGCCTCCACCTTCTGAGTGCTGAGATTACCACGAAGTGC
CAACGTGGCGGGTTTATGTTGGGCTTCAAATCAAACCTTTGTGCATGCCAGGGCATCACACTACTGAGCCATATCCTCAGTCCCATACATCTTTCAGT
GTGTGTGTGTGTGTGTTGGTGTATGTGTGTGGTGTGTTGGTGTGTGTGTGTGTTGGTGTGTGTGTTGGGGTGTGTGCAGGTACTTATGGAGCCAGTGA
TTAACCTCCAGTGTCATTCCTAAGGCACTGTTCACCTGGGAGTTTTTTTGAGACAGAGTCTCTCATTGAACCTGGGACTGCTCAGGCTCGCCTCCTGG
CTAGACAGCCTCACGAACTGCCTCTCCTGTGTGAGGGTTCCAAGCATGTACCTCAACACATGCCATTGAGCATAGGTGCTGGACTGATCTGAGAGCCT
CAAACATTTACAGTGTGTACTTGACCGATTGAGCTATCTCATATATTTTTAACTTTATGTGTGTCGATCTAGCTTACTGCCCTGCAGTTTTGTATACC
AAGGGTGCTCTTTTTAAGGACGTGTCAATTTTGAGGGTATTAGAAATCTCATTGTGGGGCTGATGAAATGGCTCAGCGGTTAAGAGCACTTACTGCTC
TTCCAGAGCATCACGTTTCAGTTCCAGCACCTGCATGACAGCTCCCAGCCATGTGAACCTTCAATTCTAGGAATCTGATGACCTTCTGGCTCTCATTG
GTACTCTCTGCACACGGTGCAGATACATATATGGCAGCCAAAACACACACACACACACACACACACAAATCCTTCAAAATAGGAAGGAAGGAAGGAAG
GAAGGAAGGAAGGAAGGAATTCCCTAGTGAGGTTTAGTCTTGAATCAGGAGTTTGCTCTGCAGAACCAGATGTAATCTTAGTTACTACCAACCAGGGC
TTTATCCTTAGGATCCCAAGGCGTGAAAGGTAAAGAGAGGAAAGCTGACTGGAGAAGTGGTGTTACTGTTATAAGGAGAGGGGAGTAGACGCAGAATC
TGAGAAGTGTGAAGCTATCACCTGATATTTAGGAGGTTTATGCTTGGAGGGGGCTTTCTTTTCTTTTCTTTTCTTTTCTTTTTTCTTTGTTTTTTTTG
TTTTTCGAGACAGGGTTTCTCTGTGTGGCCCTGACTGTCCTGGAACTCACTTTGTAGACTAGGCTGGACTCGAACTCAGAAATCTGCCTGCCTCTGCC
TCCCAAGTGCTGGGATTAAAGGCGTGAGTCACCACGCCTGGCTGGAGGGGGCTTTTTAAAAAGGGCGTTGACTATTGCCAAGTGGAGCCCCATTCTTT
TCCTGACTGTGCCTTTAAAAAAAATCGGGTTGAGCTATCTAGTTCCGACTAGCCTATTTCTTTTTTTTTTTTTTAAAGGATTTATTTATTTACTTTAC
ATGTATACTTTATATGAGTGCACTGTCACTATCTTCAGACACCAGAAGAGGGCATCCAAATCTCATTACAGATGGTTGTAAAGCCACCATGTGGTTGC
TGGGATTTGAACTCAGGACCTCCAGAAAAGCAGTCAGTGCTCTTAACCACTGAGCCATCTCTCTAGCTCCCAGACTAGCCTATTTCTAAACCTCCTAT
CTCAGCCTCTTGAGTGCTGGGATTACATACATGCTTGGGTTTCCATGGCCAGCTCCTGAGTCTTATCCTCTAATGTGTATTAAAAACTTGAACTTGGC
CAGGTATAGTGGTGTGTGCCTTTAGTGTCAGCACTCAGGAGCCTGGAGCAGGCAGAGCTCCATGAGTTCAATACCATAGAGAACTAACATAGTGAGTT
CTAGGATAGCCAGGGTTACATAGTGAGACCCAGGGGAAAAATATAACCTAACCACGACTATTAGCAAACCTTTCCCTCACTTCCATATCCTATCCACC
TCCCACTGCAGGCCGGAAATAACCTAAGACTTTCAGGAAGGAAAAATTAGTTGTTTCAAAGACCTGGGCTGTGGTGGCACACACCTAATCCTAGCACT
TGGGAGGCAGAGGCAGGTGGATTTCTGAGTTTGAGCCCAGACTGGTCTACAAAGTGAGCTCCAGGACAGCCAGGGCTAAATAGGAAAACCTTATCTAG
AAAAAACAAAGCAGAAAGAAATGGAGGACAGATAGACGCACTGACATATTATTGGGTTAGATTCTTATCACTGCTGTTTGTAAACCATCCATAATGCT
TGTCACCCAAGCCAGGTGTGGTGCCTCATGCTTGTCAACCCAACACTTGGGAAGCAGAGGTAGGAAGATCACAAGTTTGAGGCCAGTGTGACCTGTAA
GTCCAGGCCAGCTTTCGGCACCTGTCTTGGCATGGCACATCTGGCTAAGAAATGAGAGAACACTGCGAATCATCTCTAGTCAGACTCAGTTAATGTAG
ACAGCAGGGAAGTTCTTGGCAGTGATGGTGGACTGTGGAGCCAGCTAGGTGGTTAGGCTGGTCATTGATTCTATCCCTTGGCTTATTTAATAAAGGAT
GCAGTGTCATTGCTGTGCTCAATGTTAGGAAACTATAAAGTCCTATTAGGTGCCAGATAACAGAGACTTGGGTGCACCCCGGAAGGTGCAGGAGCTCC
TGTCAGAAAGGCAGCATAGCTAGAATAGTTTCTCAGCTGACATTAAGTCTCCCGTGTAGCCCACACTGGTCTTAAGCAAAGGGACAGAACGTACCGTG
CCAAGCCTTCTGCCTCTGCTTCCCAGGTACTGGCACTGCAGTTTTATATGCCACCATGCCATCTCCTTTCACACGTTAATCCACTCTATAACTTTAGC
CTGAGACAGTCCCCTTGCTTTAAAGATCTTACTGAGATATGACAGATAAATACATAAAAGTGTGCAGCTTGCCTTAGTTTCACAAGCTGCACACACTT
AAACAAGCAGCACCCAGATCCACAACAACTCAGCATCGATAACCAAGATGTGCAACCTTGTACCTGCCTTAAGTTTATGGTGTGGTAGAGCCTGGGGG
GCCTTCTCAGACCTGGTTCTAACAGACTTGTGTTGCACAGATCTGCTTTTACTATACAGTTGAGTCAACTCATAGTGAGGCGGCCTCTCCATGGAATT
CTATGGAATTCTACATTTATGGTTCCCAATGCCATAGTAGCTTCTAGTGTGAGCGAAAGGTACAAAGCTATGCTCTTACTGTCCTCTCTCTCAGTGAC
AACTCCGAGGTAAGTAAGCAGCCTCACTCTAGGGACAGACTGGACACCTGGGGTGTGTGGAAGGTCCATCATTAGCACCTGCTCATCCTGTGGGCACT
TAGCTACATTGTACTAGTTTCTTTTAAATTTTTATTTTTGCTTTTTTTTATATGAGGCAAAGTCTAGGAGTATAGCCCAGGCTGGCCTAAGATTTGTA
GATCTCCTGTCTCAGCCTCTTACACTGGGCATTCCAATTTGTTATTAAAGCTGTAAGAAAGGACATGGTGGCCAGGCTGTGGTAGTGCATGCCTTTAA
TGCCAGCATGTGGAAGGTAGAGGTCAGTGGATCTCTGTGAGCTCAGGCAGACAACCTCAGCTCAGTCCCTTGGTCCCATATAGTGAGAGAAGAAAGCC
AGCTCCCAGATGTCGTCCTCTGACCTCCGCGTGTGCACCACTGTACACATTCTCTACCCACCATGTAAAATGTTTAAGTTTCAAAAGCCAAAGCAAAG
CACAAGACACTAAGAAGAACAGGGATGAAAGGAATGGATAGTAAAAAGTGGTACATTAGCTAGATTTAAATCTTTTTCTAGCTAGCAGGATTGGCAAT
AACTGATTAGTCCTTGCAAGTTAACAACCATCATTTTTCCTGGCCCTTTACCCCTGACTGGGGCTGGGAGGGGCTGGAGGGGGTAGAGAGGGTGGGGA
GGGGTAATCGCTACCCTCTAACCAGCTCCATAGCGCAGCTGCCTTGTTCAACCCACTCTCTCACGTGGTTGGGGAGATCTTTACATGGATGTCCCTTA
AGTAGACCTTTTCAGATCTTTGGGTAAAATATACATCTTTCTTTCTTTCTTTCTTTCTTTCTTTCTTTCTTTCTTTCTTTCTTTCTTTCTTTCTTTCT
TTCTTTCTTCCTGTCTGTCTTTCTTATTTATTTATTTATTTAATTTTATTTATTTATTTATTTATTTATTGTATATGAATACACTGTAGCTGTCTTCA
TGCGTGCCAGAAGAGGGCATTGGACCCCATTACAGATGGTTGTGAGCCACCATGTGGCTGCTGGGAATTGAACTCAGGACCTCTAGAAGAGCAGCCTG
TGCGCTTAACTGCTGAGCCAACTCTCCAGTCCAAAATATGCATCTTTCACTTTTGCACTTCTGATAAAATTACCCCAAAGCCATGGAAGAAATGAGTG
TATGATTTTGTGATGAAGGAGAGGAGTTGAGACAGAGATGAATTCCCTTTGGTTTTCCACATTCCCTACCCGAGCCCCTTGTAGCCTAGGTCAAGGAG
TCCTTTGGTAACCTGAACAAAGGCTGTCACCCTGGGGCCTGCTTGTCCTTGGGTTTCTCTCTACTGCTCACACCTTGATGTCTCAACTCTGAGTTGAA
CATCTGCTTGAAGCTGCTGTGGGAGAAAGGCTACTGAGGGAAAGGGCTCACTGCAGGGGACAGTTTGCCCAGTCATTCCTCCACAGCTTGCTGTGAAC
CCTGTCAAAGGAGAAGCGCTCTGTCAACTGCCAAAATCCCCT
MOUSE SEQUENCE—mRNA
GTTGTTTCTTGTTGAGATTGGAGGAAAGACGGCCTTCTCAGAGAGCCTGACTGGAGACAGGTGTTAGGCTTGAAGCCTTCGTGACCATCCAGGAAGTT
GGACAATGGCAGCCGAAGTGCCAGCAGTGAGCACTCCCCTCAGCCCTTTGGTTCAGGTACCTCAAGAAGAAGATGAACAGGCAGAGGTCACCACTATG
ATCCTGGAGGATGACGCGTGGGTGCAGGAAGCAGTGCTGCAGGAGGATGGCCCTGAGTCTGAGCCCTTTCCCCAGAGTGCTGGAAAAGGCAGCCCCCA
GGAGGAGGACGCAGCCGAGGGACCCCAGGGTGCTCTTGTCCGATTTCGGGAGCTCTGTCGGCGCTGGCTGAGGCCAGAGGTGCACACTAAGGAGCAGA
TGCTAACTGTGCTGCCAAGAGAAATTCAGGCCTGGCTGCAAGAACATCGGCCTGAGAGCAGTGAGGAGGCAGTGGCCCTGGTGGAAGACCTGACCCAG
ACTTTTCGGCACAGTGATTTTGAGATACAGAGCGAGAATGGGGAGAACTCAAATGAAGACATGTTTGAGGGTGTGGAGTCACATGGGATGTTCTTGAA
CATCTCTGGAGGGGAAGGTGGTCAGCAGTCTGATGGGGACAGTGACTTTGAGAGAGACTGTGGCTCTGGAGGCGCCCAGGGACATGCCCCGGGTGAGG
ACCCCAGGGTCGTGCCATCGGAAGGAAGGGAAGTTGGCCAGCTAATAGGCCTTCAGGGCACCTACCTGGGTGAGAAGCCGTATGAATGTCCCCAGTGT
GGGAAAACTTTTAGCCGGAAATCCCACCTTATCACCCATGAGCGGACCCACACAGGAGAAAAATACTACAAATGTGATGAATGTGGGAAGAGCTTTAG
TGACGGCTCGAACTTTAGTAGACACCAAACGACTCACACTGGAGAGAAGCCCTACAAATGCAGGGACTGCGGGAAGAGCTTTAGCCGGAGTGCGAACC
TTATCACGCACCAGAGGATCCACACCGGCGAGAAGCCTTTCCAGTGTGCCGAGTGTGGCAAGAGTTTCAGCAGGAGCCCCAACCTCATCGCCCATCAG
CGCACGCACACAGGGGAAAAGCCGTACTCGTGCCCCGAGTGTGGCAAGAGCTTTGGCAACCGGTCCAGCCTTAATACTCACCAGGGCATTCACACCGG
AGAAAAACCCTACGCGTGCAAGGAATGCGGCGAAAGCTTCAGTTACAACTCCAACCTGATCCGACACCAGAGAATCCACACGGGAGAGAAACCATACA
AATGCACCGAGTGCGGCCAGAAGTTCAGCCAGAGCTCCGCGCTCATTACGCACCGGAGAACGCACACCGGGGAGAAGCCCTATCAGTGCGGCGAGTGC
GGCAAGAACTTCAGCCGCAGCTCCAACCTGGCCACTCACCGGCGCACCCACCTGGTGGAGAAGCCGTACAAGTGCGGGCTGTGCGGCAAGAGCTTCAG
CCAGAGCTCCAGCCTGATCGCGCACCAGGGCACGCACACCGGCGAGAAGCCCTACGAGTGCCTCACGTGCGGCGAGAGCTTCAGCTGGAGCTCCAACC
TCATCAAGCACCAGCGGACGCACACCGGCGAGAAGCCCTAAAGATGCGGCGACTGTGGGAAGGGCTTCAGCCAGCGCTCGCAGCTCGTGGTGCACCAG
CGGACGCACACCGGCGAGAAGCCCTACAAGTGCCTCATGTGTGGCAAGAGCTTCAGCCGGGGCTCCATTCTGGTGATGCACCAGCGAGCGCACTTGGG
AGACAAGCCTTACAGGTGCCCGGAGTGCGGGAAGGGCTTCAGCTGGAACTCCGTTCTCATCATCCACCAGCGCATCCACACGGGAGAGAAGCCCTACA
GATGCCCGGAGTGCGGCAAAGGCTTCAGCAACAGCTCCAACTTCATCACACACCAGAGGACGCACCTGAAAGAGAAGCTTTACTGAAGTGGCAGAAAA
GAGAAGGAAGTGCTGAGCTGACTCTGCAGGGAGAACTCCCACAGTGTCCCTCCCCACAACCCTCCCCCCACAACCCCCCTCCCCCCCGCCCGTGTTGT
CCCTTTAAAAGAACCACTTTTTCCTAAATAAAAAAA
MOUSE SEQUENCE—CODING
ATGGCAGCCGAAGTGCCAGCAGTGAGCACTCCCCTCAGCCCTTTGGTTCAGGTACCTCAAGAAGAAGATGAACAGGCAGAGGTCACCACTATGATCCT
GGAGGATGACGCGTGGGTGCAGGAAGCAGTGCTGCAGGAGGATGGCCCTGAGTCTGAGCCCTTTCCCCAGAGTGCTGGAAAAGGCAGCCCCCAGGAGG
AGGACGCAGCCGAGGGACCCCAGGGTGCTCTTGTCCGATTTCGGGAGCTCTGTCGGCGCTGGCTGAGGCCAGAGGTGCACACTAAGGAGCAGATGCTA
ACTGTGCTGCCAAGAGAAATTCAGGCCTGGCTGCAAGAACATCGGCCTGAGAGCAGTGAGGAGGCAGTGGCCCTGGTGGAAGACCTGACCCAGACTTT
TCGGCACAGTGATTTTGAGATACAGAGCGAGAATGGGGAGAACTCAAATGAAGACATGTTTGAGGGTGTGGAGTCACATGGGATGTTCTTGAACATCT
CTGGAGGGGAAGGTGGTCAGCAGTCTGATGGGGACAGTGACTTTGAGAGAGACTGTGGCTCTGGAGGCGCCCAGGGACATGCCCCGGGTGAGGACCCC
AGGGTCGTGCCATCGGAAGGAAGGGAAGTTGGCCAGCTAATAGGCCTTCAGGGCACCTACCTGGGTGAGAAGCCGTATGAATGTCCCCAGTGTGGGAA
AACTTTTAGCCGGAAATCCCACCTTATCACCCATGAGCGGACCCACACAGGAGAAAAATACTACAAATGTGATGAATGTGGGAAGAGCTTTAGTGACG
GCTCGAACTTTAGTAGACACCAAACGACTCACACTGGAGAGAAGCCCTACAAATGCAGGGACTGCGGGAAGAGCTTTAGCCGGAGTGCGAACCTTATC
ACGCACCAGAGGATCCACACCGGCGAGAAGCCTTTCCAGTGTGCCGAGTGTGGCAAGAGTTTCAGCAGGAGCCCCAACCTCATCGCCCATCAGCGCAC
GCACACAGGGGAAAAGCCGTACTCGTGCCCCGAGTGTGGCAAGAGCTTTGGCAACCGGTCCAGCCTTAATACTCACCAGGGCATTCACACCGGAGAAA
AACCCTACGCGTGCAAGGAATGCGGCGAAAGCTTCAGTTACAACTCCAACCTGATCCGACACCAGAGAATCCACACGGGAGAGAAACCATACAAATGC
ACCGAGTGCGGCCAGAAGTTCAGCCAGAGCTCCGCGCTCATTACGCACCGGAGAACGCACACCGGGGAGAAGCCCTATCAGTGCGGCGAGTGCGGCAA
GAACTTCAGCCGCAGCTCCAACCTGGCCACTCACCGGCGCACCCACCTGGTGGAGAAGCCGTACAAGTGCGGGCTGTGCGGCAAGAGCTTCAGCCAGA
GCTCCAGCCTGATCGCGAACCAGGGCACGCACACCGGCGAGAAGCCCTACGAGTGCCTCACGTGCGGCGAGAGCTTCAGCTGGAGCTCCAACCTCATC
AAGCACCAGCGGACGCACACCGGCGAGAAGCCCTACAGATGCGGCGACTGTGGGAAGGGCTTCAGCCAGCGCTCGAAGCTCGTGGTGCACCAGCGGAC
GCACACCGGCGAGAAGCCCTACAAGTGCCTCATGTGTGGCAAGAGCTTCAGCCGGGGCTCCATTCTGGTGATGCACCAGCGAGCGCACTTGGGAGACA
AGCCTTACAGGTGCCCGGAGTGCGGGAAGGGCTTCAGCTGGAACTCCGTTCTCATCATCCACCAGCGCATCCACACGGGAGAGAAGCCCTACAGATGC
CCGGAGTGCGGCAAGGCTTCAGCAAAGCTCCAACTTCATCACACACCAGAGGACGCACCTGAAAGAGAAGCTTTACTGA
HUMAN SEQUENCE—GENOMIC
TTTGAAGCCAAAGCCAGGCTACACAGAGATGAGGCTCCTGGGAGGGAACAGGTGCAGTCCTGCTCAGGCTGCAGTGTCCCTGGGCTACAGCTGGACAA
GGGCTCCCTCTGGGTCACAGCCTCTAGGGACAGAGTTTGGGGCAGGTTGACTGTCTGATTTGTAGGACTCTGGTGATGAGCTCAGCTGGGAAACTGTG
TGGTAATGGGAAGAGAATGGTTTTGCTTTTCCAAATCATCCACCCTCTGCATGCTTTGATCCATACTGGAACTCAGTGTGGAAACAGTGCCTTTGAAC
AGCCTAAGCCTAAACTCTGGGCTGTGTGTCCAGTTTCCTGGCCTCCAGCTTAGTTCACTCCTTACCCCGCCCATCACTGATACCAGATCCATCCTCCC
AAATATCCTTTCATCATGTTACCTCCCTACCACCTCATGGCACCTTCAACACAACAGGAAATGTCTCCAACCTCAGCCTGGTATTCAAGGTTCTTTCT
GATGTGGCCCACCTCTCACCACTCCACCACATCTCCCTGTGGGCCCTGCCAAACTGGATCATTTTTTTTCTATCCATGAGCTGTGAACTTTCCCACCT
CCAAATTGTTGCTTATACTGTGTCCTTCCTCTGAATGTTCTCTTCCTCCCTCTCCCCCAGTCCAAATGCTATAGACTGAATTGGGTCTCCCCAAAATT
CATACATTGGAGCTCTGATCTGCTATGTGATTGTATTTGGAAATAGAGCTCTTAGGAGATAATTAAAGTTCGGTGAGGGAGGAGGAGCAGATCAAGAT
GGCTGAATAGCAGCCTCCACCAATGATTCTCCTTGCAGGAAAATCAAATTTAACAACTGTCCACAGCAAAAAAGCATCTTCATAAGAACCAAAAATCA
GGTGAGGACTCAAAGTACCTGGCTTTAACTTCATATCGCTGAAAGAGGCACTGAGGAGGGTGGGAAAGACAGTCTTGAAACACTGATGTCACCCCTCC
CCCATCCCTCAGCAGTGGCCGCTGTGGTGCTGAGAGAATCTGTGCACTTGGGAGAGGGAGAATGCAGCAACTGTGGGACTTTGCACTGAACTCAGTGC
TGCCCTGTCACAGCAGAAAGCAAAACCAGGTGGAACTCTCATGACACCTGCCCATGGAGGGAGCATTTAGACCAGCCCTACCCAGAGGACTTCACCCA
TCCCAGTAGTTAGGAGGCTTGAGGCTTGGGTTTTGACAAGCCTTGGCATGGCAGGCTATCATGCTCTGGGGCCCTAAATAAACTTTCAAGCCAGTCTA
GACCATAAGGACTTCAACTCCTAGGCAATTCCTAATGCCATGCTGGGCTCAGAGCCAGTGGACTCGGGGCACACAACCTAGAGAGACACCAGGCAAGG
GAGCCAAGGGAGGGTTTGCACCACCCCTCCCCCAACCCCAGGCAGCACAGCTTGCAGCAACAAAAGTGACTTCTTCCTTTTGCTTGAGGAGAGGAGGG
GGAAGAGTAAAGAGGACTTTATCTTACATCTTGGATACCAGCTCAGCCACAGTAGGATAGAGCACTAGTCAGAGTTGAGAGGCCTCCATTCCAGGACG
TAGCTCCTGAATTACATTTCTAGATACACCCTGGGCCAGAAGGGAACCTGCTGCCTTGAAGGAAAGAACCCAGTCCTGGCAGGATTCATCATCTGCTG
ACTCTAGAGCCCTTTTGACCCAAATAACCTGCAGTGATACCCAGGTAGTATGCTATGGGCCTGGAGTAAGACTCTGAGGCATGCTGGCTTCGGGTGTA
GACCCAGCATATTCCCAGCTATGGTGGCTGTGGTGAGAGACTTCTTCTGCTTGAGAAAAGCAGAGAGAGATGCACAAGGGACTCTGTCTTGCAGCTTA
GGTACCTGCCTGGCCACAATGTGGTAGAGCATCAAATGGGCTCTTGTGGTCACTGATTCTAGGCCTTGGCTCTTAGACAGCATTTCTGGACCTGCTCT
GACCTCACCCTGAAGGGTGAGTCCCAGGCCTGGAAGCATTCACCACAAGCAACTGAAGAGCCCTTGGGCCCTAAGTGAACATAGCCAGTAGCTTGGCA
GTACTCGCTGTGGGCCTGTGGGGGTGGCCACAGGGTGAGGCTCCTCTGCCTGTGGAAAGGGGAAGGAAGAATGGGAAGGACTTTTTCACATGGTTTCA
GTGCCAGTTCAGCCACCATAGACTAAAGCACCAGGTAAATTTCTGAGGTTTTTGACTCCAGTCCCTGGCTCCCAGACAGCATCTCTGGACCCGCCAGG
GCCTGAGGGAACTCATTGGCCTGAAGGGAAGGACACAAACATGGCTGGCTTTGCCACCTGCTGACTGTAGAGCCCTAGGCCTTGAGTGAACATAGGGG
ATAGCCAGGTAGTGTTTACAGCAGGCTTTGAGTGAGACCCAGTGCTTTGCTGGCTTTAGGTCTGATCCATTGCAGTCCCAGTGGTGGTGGCCATAGGG
ATGTTGTGTCAGCCCACCCCTAGCTCCAAGTGGCTCAGCAGAGAGAGAGAGACTGAGACTGTTTGTTTGGGAGAAATGACAGGTAGAGGATAAGAATC
TCTGCCTGGTAGTCCAGAGAATTCTAGATCTTACCCAAGACCACCAAGGCAGTACCTCTATGAGTCTGCAAGAACCATAGCATTACTGGGTTTGGCGT
GTCCCCTAATGCAGATACAGTTTAGATCACAAGACCCACATCCTTCAAATACCTGGAGAGCCTTCCCAAGGATGGGTACAAACAACCCCAGACTGAGA
AGACTACAATAAATACATAAATCTTGAATGCTAAGGCACTGACAAACAACTGCAACCATCAAGACCATCCAGGAAAACATGACCTCACCAAACAAACT
AGAGGGGCGAATCCTAGAGAAGCAGAGATAAGTGACCTCCAGACAGATAATTTAAAATAGCAGTTTTGGGGAAACTCAGAGAAATTAAAGATAACACA
GAAAGGATTCAGAATTCTATAAGATAAATTTAACAATGAGATTGAAATAAAAAGAATCAAGCAGAAATTCTGGAGTTGAAAAATGCAATTGACATAAT
GAAGAATGCATCAGTCTCTTAATTGTGGAATTGATCAAGCAGAAGAAAAAATTAGTGAGCTTGAAGACAAGCTATTTGAAAATATACAGTTAGAGAAG
ACAAAAAAAAATTTTTTTTAATGAAGCATGCCCACGAAATCTAGAAAACAGCCTCAGAAGGGCAAAGCCAAGAGTTATTGGCCTTAAAGAGGAAACAG
AGAAATAGAAAGTTTATTCAAAGGGATAATAACAGAGAACTTTCCAAACCTAGAGAAAGCTATAAATATTCAAGTACAAGAAGATTATAGAACACCAA
GCAGATTTAACCCAAAGAAGACTACCTGAAGACATTTAATACTCAAACTCCCAAAGGTCAAGGATAAAGAAAGGATCCTAAAGCAGCAAGAGAAAAGA
AACAAATAACATACAATGGGACTCCAATACGCCTGGCAGCAGACTTTCTGTGAAAACCTTACATGCCAGGAGAGAGCAGCATGACATATTTTAAGAGC
TGAAGGAAAAAACTTTTACCCTAGAATAACATATCCAGTGAAAATGTCCTTTGAACATGAAGGAGAAATAGACTTTCCCAGACAAACAAAAGCTGAGG
GATTTCATCAACACCAGACCTATCCTACAGGAAATGCTAAAGGGACATCTTCAATCAGAAACGAAAGGACATTAATGAGCAATAAGACATCATGTGAA
GTTACAAAACTCACTGGTAATAGTAAGTACACAGAAAAACACAGAGTATTATAACACGGCAATTGTGCAAACAACTCTTAAGTAGAACGACTAAAAGA
TGAACCAAACAAAAATAGTAATAAGTACAACAACTTCTCAAGACATAGACAGTGCAATAAGATGTAAGTAGAAATAGCAAAAAGTTAAAAAGCAGGAC
AAATTAAAATAGAGTTTTTATTAGTTTTCTTTTTATTTGTTTCATTGTTTATTTGTTTGTTCCTTTGTGCAAGCAGTGCAAAGTTGTCATCAGTTTAA
ATAATGGGTTATAAGATGGTATTTCCAAGCCTCATGGTAATCTCAAATTTAAAAATATGCAATAAATACACAAAACACAAAAAGCAAGAAATTAAATC
ATACTACCAGAGAAAATCACCTTCACAATAAGGAAGACAGAAAGAAAAGAAGAAAGAGAAGACCACAGCAACCAGAAAACAAAATGGCAAGAATAAGT
CAATACTAACATTGAATGTTAATGGACTAAACTCTCCAATCAAAACTGTTATGGAAAGAACTGTTATGGAGCAGTTCTCCCCATTCCCACCCACATCA
TTTTTCATCAACTCTAGATGAGCTAAGGGCCCCCAAATGCCTTAGGCTGAGCATTCCTTGGGGTGAGGTCCCTTCTTGCTCAGAGCCTAAAGACAAGT
AGTGATATTGTTTCTGCCTGTCCAGTGTTTCCTAACAGGCCTTCAGTCTCCAGAGTGGAGAATCTGCCTTTGTGTGGGGTATTAGTAAGAAAATCTAG
GAGCCACATCCAATCCTCTCTCACCCTGCTGCATCCAGGGAATGTGCATGTGACTTAGACTCAACCAACCAAATGCTCTTTCCTTGCATGGGAGAATT
TGTGTGAGAAAGGCGGAGAAACAGAGATGGCTGTAGTACCACCAGAGGTCATGGGATAGGCAAGCTGAACTTTTTCTGCTCAGAGACTGTTATTGTGG
TTTTTGGGGACTTCCTCTTTGTCTCTTGGCTCCTAACTGTTCTCAAGCCTGATTCGTCAGCTCCCTTCTACTCTGTCAGTTCCCTGACAGTCTTTCAG
TAAGTCCCTTTTACTTAAGTTAGCCAGATTAGTTCTTAAGCTTTTAACCAAGAATGCTTTCTGGTGGAACAGAGCCTCTCTCACCCTTGTGGTTTGGC
CAATGGAGAAAGGACTCTCTTGGGGGAGCAACCTTGAACTTGGCTGTGTTTCCAGGCTTGCTTCGATGTAGCAGGCTGGTCTTAGGGTACTGGCTTGG
CTACCAGGGCCCTTCTCAGTAGGATCATTATGCCATGAATTGGTCTTAAATGTGACTTTCTCTCTGTCTCATGTAGGATAGTTGACACTGGCAAGGAC
CAGCTTCCCAGAATCAGACACAGAGGAGACAGCTTCTTTAGGACTCTCCAGTGACAAGCTAGTGCCAGTTCTGTGGTCAAGGGTTGACATGGGTTAGG
AACAGAGGTAAATAAACGCTTGAGCTGCTGGAAAGTTCCATCTGTGCATCCCCAGCCTCCCCTGCATCTTTTCCTTTTTGTGTAAATTTCCATTCCTG
GTAAGGATGGACACATGGAGAAACCCATCTTTTTTAATACAATGTCATCCCAAAAAAAATCACTTGGAGGGGTGGTGGCCCAGAATTCTGCATCCAGG
GGCTTAGTGCAGACTTCTCAGGCTGGCCTGGAGGTGGTGCTATTTGCATGTGGTCAGTCTGGGATAATAAAGATGCTACCAGGGTATTGGATAAGAAT
CACTAGCTGAACTTCACTTTGCATCTCTGTAATTGGATTCAAGAGGTTAATTAGTGAAAAACAGGTGAATTTCACATAATCTTTGGAGTGGTGCAGAG
GACTTTCTAACACAAAACTCACAGGCTATAAAAAGATAAGATTAACAAATTTGACTGCAAAAAAATTTCAAATTTCTATATTTAATAAACCAGCAGAA
AACAAACAGCAAACTGAGAAAGAGTAGCAACTGCTGTGACAAAGGGATAATTTTCTTAATATACAAAGAGCTCTTACAAATAAATCCAATTTAAAAAT
GGGAAAAGGACCGGGCACGTGGCTCATGCCTGTAATCTCAGCACTTTGGGAGGCCAAGACGGCTGGATCACCTGAGGTCAGGAATTCAAGACCAGCCT
GATCAACATGGTGAAATCTCTGTCTCTACTAAAAACACGCAAAATTAGCCAGGTGTGGTGGCGCACGCCGGTAATCCCAGCTACTTGGGAGGCTGAGG
CAGGAGAATCACTTGATCCGAGATCGTGTCACTGCACTCCAGCCTGGGCGACAGAGGAGGAAAAAAAAAAGCAGTCATTTCATTGCAGGTGCCTCCAG
TAATTGCTCAGTGCAAACATACATAACTATTCTATCACAGACCATTAAGGGTGTCTCTCTCCATTTTTAGATTTTCCGCTTGAGTTTCCAGGTAGTTT
CTCACCTGTTTTCAAGCAGGGTTGGGGCAGCCGTTTCTTCAACAGCGCCAGCCGTAAAACACAAAGATGGAGGCCAACCTCCCACACCCGGGCACCCT
CCCTTCCCCCATACTGAGATTGACAAGTAAGCAACCCGGTGATCAGTACTAAAATGGAGACGTCAGCCAGGTGCGGTGGCTCACACCTGTAATTCCAG
CACTTTGGGAGGCTGAGGAGGAAGGATCGCTTGAGCCCAGGGGTTCGAGGTCAGCCTGGGCAACATATGGAAACCCTGTCTCTATGAAATGTAGATCC
TGTCTGTGAGTGTGTGTGTATAAATAAATATATATATATATATACAAATACATACATATATATATTTAAAAAATAAAATGGGGACGTCCAACACGTGT
ACTAGGGGCGGTGTCTCTGCCTGGGGAGGTGGTGATGACAGGGCAGGCTCCACCCAGGAGAAGCTGGAAGAATGGGAGTTCCGCGGCAGAGGAGGCGG
GGCAGGGCGTTCCTCCAAACACAGGAATACCACGTGCGAAAACAAAGGGGTGTGCGAAGACCGGGTGCATGTGTACAGCTGCAAACTGGGGGAAAGGC
GCCAGGCTTGGGCGATGAGGATTTGTTCCCCCATGGATTCCCTTCCTGCCCAGGGTCCGCTCTCGCCGGTCGAGGGTGCCTAACAATACTCCTTGCCA
CAAGGTTCGCAGCCGAAAAGGTCCTTCAAGTCCGGCCATCCAACTTTCACTTTTTATAGACAAATATACAGAGGCCTTAGATAAGTGAAACGGCTTGT
CTCGGGTTACAGCTCTAGCCAACACAGGGTTTCTGACGGTCAGTCTGCGGATTTTCCTGCCTCTGGTCCGCAGGCACTGCCCAGGCTGTCAGCCCCAA
ACCCTACTCCGGGGACCGCGGTCAGGTTCGTCTCCGGGCGGACTACATCTCCCACAATGCCTTGGGCCCAGCCTCCCTCCTGCCGCCCGGCTGGGTGC
CGTCTCCACCAACAGAAAGGCAGAATTTCCAGGGCCGTTCTCGGCAGCCAATGAGCGCGGCGGTGGGCGGGCCTCTCCCGTCCATTGTTCTCGGTGCC
CCACGGGCTTGAGCCGGGGTGAATCTGGAGGGGCCGGGCCGAGCCCGGGGGCGCTTTCGCACGCGAAGCAACCGCTAGAGCAGGACCTGGTCTCCCGA
GAGGTGAGCCGGAGAGGCAGGCCTGGAGCCACGCGGACCCCGGGCAGTAGCCCGAGGTGAGGGAATAGTGGGCCTGGAGCCAGCTGCCGGCAGCTCTG
CTGGGGGAGGGGCGTCGGGGTCGCGCTCCGTATCCTGCGGGCCCTGCAGCCCCGATTTACGCGCCGGCTCCGCTCAGCGAACCGTCCCGGACGCGTCT
CCCTGGCGGAGAGCTCCTTGCCTCTCCTACCGAGAAGCGCAGGCTTTGGACGGGAAGGGGCATTCGACGACATCCCGCGCAGCGTACACGTTTTACAG
ATGGGGAAACTGAGGTTCAGAGGGGGCGTGTCGTGCCCCAAGTCAGACCGCAAATTAGCTGACCTGGCACACTTTTCCCAGGCTCCCAAGTAGGAAGA
GTTCTCTTACACCATATGCCAGTATTTCTTCTAGTCATTTGAGTAAATACACGTTGAGAGCTTCGGTTTTCCCACTAGCTTTTATTTTTTGTCCTTTT
TTTTTTAAGCTTTTGTCAGCTTTTCTCCCCTTTTCATTGAGGGAGGGTTGTTAGTTGAGTCTTTATGATGTAGTAATGATTTTTTAAAAAATTTTTTT
ATTTTTGTAGAGACTGGGTCTCACTCTGTTGCCCAGGTTGGTCATGAACTCCCGGCCTCAAGCCATCTTCCCTCCTCGGCCTCCCAAAGTGCTGGAAT
TACAGGCGTGAGCTACAGCGCTTGGGCTAAAAATAATTTTGTATTGCTTTTATAATATTTTAAGTCGTTTGCAATTCCACTGCTTTCAATATTTTGTA
GGTGCCGTCCCGTTCTTTTTTCCTGTGAGTTCATTTTTTTTTTTTTTTTTTTTTGAGACGGAGTTTTGCTTTTGTTGCCCAGGCTGGAGTGCAATGGC
CTGATCTCCGCTCACCGCATCCTCTGCCTCCCGGGTTCAAGCGATTCTCCTGCCTCAGCCTCCGGAGTAGCTGGGATTACGGGCATGCGCAACCACGC
CTGGCTAATTTTGTTTTTAGTAGAGACGAGGTTTCTCCGTGTTGGTCAGGCTGGTCTCGATCTTCCGACCTCAGGTGATCCGCCCGCCTCAACCTGCC
AAAGTGCTAGGATTACAGGTCTGACCCACCGCGCCCGGCTGTGAGTTCATTTTTAAAGGGAATTCAGGACTTACTGTGTCTATGGTTTTTCTGCCCTG
CTGATTTTGTGTTTATCCATTATTTTCCTATCAGAAGGATTATCTTAAATCATTAATGATTAATCTTTGTCAATATAATTTTTAAAATATTAAAAACA
TACAGCGTAATGTGCAGATCTTAAATTTCATATGTTTGCTCTTGTGCCGCCATTGCCAAATGAAGATATTGAAAATTTCCATCATCCCAGAAGGCTCC
TTTGAGCCCCTTTCCTGTCATTACCACCTAAAGGTAGCCATTATTATAGCACAGTTTCTTTTCTTTTTTTTCCTTTTCTTTTCTTTTTTTTTTTTTTT
TTTTGAGACAGAGTCTCGCTCTTTCGCCCAGACTGGAGTGCAGTGGCACGATCTCGGCTCACTGCAACCTCCTCCTCCCAGGTTCGGGCAATTCTCCT
GCCTCAGCCTCCTCAGTAGCTGGGATTACAGGCGCCCGTCACCATGCCTGGCTAATTTTTGTATTTTTGGTAGAGACGGGGTTTCACTATATTGGCCA
GGCTCGTCTCGAACTCCTGACCTCAAGTGATCCGCCTGCCGAGGCCTCCCAAAGTGCTGGGATTACAGGCAACCACGCCAAGCCTATAGCACAGTTTC
TAATACATGCTCCTATATGGGTGTGTCATAGTTTATTAGCCATTCCCTCCTGCTGGATTTTTAAGGACTCGATTTTTTGGTTATTTTTTTAAGATAAA
GTACTGAGGGGCCGAGGCTGGATTGGAGAGTATATGCATGTTTTAAACTTCATACTTAAAGTATGGTAATAATGCAGTTGAGATATTGCCTTTCTTCC
AAAGAAGCTCAGAGTGCTTTGACATCTGATCCATTGCTGATCCTTAAGATGCCCCCATAAAAACAGAAACATGAGTGTTATTATCTTTATTAGGTGGT
CACCGAGTTTGGTGACTAAGTTGTATGTTAGGTCAAGGTCACAAAGTGCAAGTCGAAACTGACTTAGGAGAGTCCTCAGTATTTCAGTCATGGGTTTT
TCCCGGGGACTTTGCATTTCTGATACTCATTCTTTGGTGTTTTGGAAGGAACAATGCTGTATTTTTTGTGCTTGCGATACTTTTTTTTTTTTTTTTTT
GAGACGGAGTCTCACTCTGTTGCCTGGGCTGGAGTGCAGTGGCGAGATCTGTGCTCACTGCAACCTCCGCCTCCTGGGTTCAAGAGATTCTCCTGCCT
CAGCCTCCCAAGTAGCAGGAACTACAGGCCCCCGCCACCACGCCCGGCTAATTTTTGTATTTTTAATAGAAATGGGGTTTCACCATACCTCAGGTGAT
CCGCCCACCTCGGCCTCCCAAGTGCTGGGATTACAGGCATGAGACACTGAACCCACCCTTCTTTTACCACAGAGAGATGTTTTCAGGAATTAAGCCAT
TTTTTCTTTAGAAGAACAGGAAACGTCTGTTGGTTTGCATATGTAAGGGCCACTTGGTGGTCTGACCTGGGCTTTTGTGAGTTAGATTGCTTTACGAT
CAGTCTACCTATCGATTATGGTTCTCTTTTTTGTTTCTCAGCGGGACTACTTGTTCATATTTGAGGAGGGAAGTGTCTTACCTGAGAGCCTGGCTGGA
GAAGACTGAGGTCCAAGGCTTGAAGCCTAAGTGATTGCCCCAGGACTGTGGATGATGGCTGCAGACATCCCGAGAGTGACCACTCCGCTGAGCTCCTT
GGTCCAGGTGCCTCAAGAGGAAGATAGACAGGAGGAGGAGGTCACCACCATGATCCTGGAGGATGACTCCTGGGTGCAAGAAGCTGTGCTGCAGGAGG
ATGCCCCTGAGTCTGAGCCCTTTCCCCAGAGTGCTCGCAAGGGCGGCCCCCAGGAGGAGCTGACCAGGGGACCACAGGGTGCACTCCGCCGCCTCCGA
GAGCTCTGCCGGCGCTGGCTGAGACCAGAGGTACACACCAAGGAGCAGATGTTAACCATGCTGCCAAAGGAAATTCAGGCTTGGCTGCAAGAGCATCG
GCCTGAAAGCAGTGAGCAGGCAGCGGCCCTCGTGGAAGACTTCACCCAGACCCTTCAGGACAGTGGTGAGACGCAGAACCTCATAGGGAGAGGGCGGG
AGCACCCTTCCAAGGTAGAGGAGTGTGGTGTTTCGGAGGAGGAGAAGGTGGTGTCCAAGGCAGAGTGGGGGGCTAGCGCCATCCCTCTGCTCTGTCTG
CAGGCAGTCAGCGTGTTCATCAGCCTTTTAGTGTCCTCACTGTGTGCAAAGTCAGCTCCAGAAGTGCTAGGAGGGCCTTAGAGCTACATTTGAATTGT
AAGCACAGTTTTTACTTCTGTGTTTATTTGGAGTGGTTGGGAAGGTGATGGAAGTTAAACGGGAGGAGATGGAGACCCCTGGCACCTTCACGTGGTTG
TGCAAGAAACATCCAAATGGTTCTTGCAATTAGAGAAAACAATCTGATATTTTCAACATGACTTTTTTTTCTTTTTTCTTTTTTTTTTTTTTTTTGAA
ACGGAGTCTCGCTCTGTCACCCAGCCTGGGGTGCAGTGGCACAATCTTGGCTAACTGCAACCTCCGTCTTCTGGGTTCAAGCAGTTCTCCTGCCTCAG
CCTTTTGAGTAGCTGGAATTACAGGCGTGCGCCACCACGCCCGGCTAATTTTTTTATTTTTCGTAGAGATGGGGCTTCACCATGTTCGCAAGACTGGT
CTCAAGCTCCTAACCTCGTGATCCGCCCACCTCAGCCCCCGAAAGTGCTGGGATTACAGGCGTGAGTCACCACGCCTGGCCCCAAAGTGGTTATTTTT
ATGAAACCAAGAGAAATGACAAGTAAACCAGCTAATAACTAGGGACTTTCTGTGGTATAAAGTAATCCTGGGCTCTTAAAATCGTAATTTCAAACTTG
AGCTCTTTGCAATAGTTACTCTATTTTTTTCCGGTGTTACTAATAAGTATTGGGATTCTGGCTTGCCGTTTAACCGCTGTCTCCCTCCTGGTTTTCCA
TTGTAAAGTAGACTCACTTGATGACCAAATGTTTACTTCAGATGTGCCTTGAGGGCTGTCTCAGGGCTCGAAGGATATGGCTGCCTTTGTGCTGATGA
AAACTACGGAAGTTCAGAGAAGGGAGCGTGGAGAGGAAAGGTCAGGTCAGAGAAGGCTTCCTGGAGGAGGTGACACCTAAGCAGGAGGTTGTAGAAGG
GAGTAGGGTGTGGCAGCTGAGAGGAGCTGTGCTGAGCAGAGGCTTGGAAGGGCAGAGCCACGGTGGTTTATACCTTTGGGTGACAGGAGAGCTGGCAG
ATTGGAGTGGCAAGTGTGTTTTTAGGAAGTGGTAGGAAGTAGAGTTTGGGGGAAGGGTGGGTGAGGGCTTGGACTGAATGTTTTGCCTCAGTGGGTTC
TTGAACTAGACCCCCAAGGGGCATGGAAGGAAGCGGAGTTTGGCAACAGGATGTAGGTTTGATTAGGGTAGTGAGAGGCTACGGGCAGGACAGAACCC
AGGAAGCTCCCTTGTCTTCAGGTGTGCAGTGATGGGGGTCTGCCCCAGATGGGGCACGTGTGAGAAAGAACTGATAGTCACCATGGCTAGCACTTTCC
AAGCTCTTTCTGCAAGCCAGGAAATATGCTTAGCATCTAATATGGAAAACTCATGTAATCTTCACCACCCTGGAAGATAAGCAGGATTATCATCACCA
GAGATCCCTTAAGATCATGTGCCTTTGTACCAGGCAGAACCAAGATTTGAACTTATGTGTGTGAGGACATAGCCCATGTTTTTAAGCGTTGTCATATG
TATGATTCTAAGAGACACTTCCTAGTGGGTGTTGGAAGTTGGGGATGCAGATGGAATTAAGACAGTGACTACAGAGCATCTGGCCTGGAGGATTCAGA
GGACTGGGAAGAGAAGAGAAGGAAACTGAAGGGGATTTGGGGCAGAGACTATCACAAACTCCAGGGACTGGGTGAATGAGTAGAGCTGTCCTCTGGAG
AAAGGGACTGACGCCGAAGGTCTGCAGTGACCCAGCCCTGCCCTGGGCACCTTCATGCTTGTCTCACTAAGAATCCTGTGAGTGGCTGGAATTGTCAT
TTCACAGATAGGAAATATAAGTGCAGGAAACTCACATGGCTGTTGAGTGGTGGAGCCAGGATTCAAACCCAGACCTGTCTCTTTCTGCATGGAGATCT
CAGTTTTGCTTGAAGCAAAGTGCTTGATTTTCTTTGGCCCTGAAAAAATAACTGCATTTTATTCCTTCTATTAAAATAAGCAAACCTGTCTCCCTATT
TTACAGCTGTAGCTGTCGTTGCTTCCCTTCCGGTGGAGGTGACCAGTTTGTAATGAACTTCTGGATCTCCAGTCAGATACTATGGAGGAGAGAGTGAT
GGAGGGCTGAGGAGAGGCCTCAGGTTGTTATTTGGTTGGGGAGGCACAGACAAGAAATTCAGGTCTCAGCAGGTGTCAGAGGTGATTAAAGCCCTTCC
CATCCACCTTGCAGCAGGGGCTTTGTGCCTTTCATCTTTAACTGGGCACCTGAGATGGGAACCAATGGGGACCATCCCAGACACATGGGTTTGTCTGT
CCCTGACTTCTGCCTCTGAGATTGTCTTTGTTGGTCTTTGTTTAATAAACGCCCCGACCTTCCTCATGATTGCTCACAGGAGTCCCTGCGTTTGTCTT
CATCCCTTCTCAAACATTTACTCGACATAAGGCCGCGTGACCTCTATTCTGTTTGTCCAGCTTATGACCTTTGGACACATTCACCCAGCACAGGATAT
TTCCAGGCCTGGTTAGTCAGTGGCGGTTTAATGTTTTTCTGACAGGAACCTCTGGGCACTCAAAATTCAAGTACCTTCTAATTCTAATGAAGATCGGA
CCTATATTAATAGATTAAGTGGCAGAGCTTATGTCTCTCTGTGACTCTTTCCAGAGGACATTTTACTCTCCATACCCAGCCCTAATCCAGATGCGTGG
AGTTTCTCACCTGAATTCTTGGAGCAGCTGCCAAACTGAACCTGTCTTCAGCTTCTCCCCCTTCATCCCAGCTCTTCAACTTCTACCAGAGTCATGGG
GCACTCCTGCTTAAAAACCTTCATGAGCTTCCCAGTATCTACCAAATCAAGCATGACCTCCTTGTTCTGCTTGGAATCCGTGGCTCTGTGGGACCTGG
CCCAAACCTCCCCTGCAGTCTTTTCCCACCCCTGACCCCCCATGCTCTATCCCCACTCCATTCAGACAAGATCTTGCTCTTTCTTGAAAACAGCCTGT
GCTTTTGTTTTTGTTTTTGTTTTTTTGAGACGGTCTTTCTACGTTCCCAAGGCTGGAGTGCAGTGGTGCCATCTGGGCTCACTGCAACCTCAACCTCC
TGGGTTCAAGTGATTCTCCTGCCTCAGCCTCCCAAGTAGCTGGGATTACAGGTGCGTGCTACCGTGCCCAGCTAATTTTTGTATTTTTAGTAGAGACA
GAGTTTCACCACGTTGGTCAGACTGGTCTCGACTCCTGACCTCAGGTGATCAAAACCCACCTTGGCTTCCCAAGTGCTGGGATTACAGGCGTGAGCCA
CCACGCCTGGCCCTTGCTCCTGGATTTTTGGCTTGGAATAGCCTTGTCCCATCTCTGTGATAAAATCCCAGTTGTTCTTTGAGTGCCCACCCGTGGAG
ACCCTCTTCATAAAGCTGCTCTTAAACCCTGTCCCTTTTCCCCATCCCCAGTTCCACACACCTGGGAGCACCTCTAATCACCTCTGCTAGAAGTCGTC
TCCCCCAGAACATTTGGATTCTCTCTCTTGGCTTTTTATCATAGTTCACCTTCTTTTCTGTTTATATGAATACTTGTCTTATTTCTTCTAGGAACCTG
TAAAATCCTAGAAGACAAGGAACCTGTCTTTTTCCTTTCTCTTTCATTCCCCCTCTTCCCTCTCTTCCACCCTACTCCCTCTTCCTTTCCCTCTCCCT
TCAGTATCATGAACCTCGGAGCCCCACCCTAGAACTCCTGAATCAGACACTTTGAGGGTGGCTGGGCGCAATGGCTCACGGCTGTAATCCCAGCACTT
GGTGAGGCCGAGGCGGGTGGATCACTCGAGGCCAGGAGTTCGAGACCAGCCTGGCCAACATAGTGAAACCCTGTCTCTGCTAAAAATACAAAAAAAAT
CAGCCAGGCATGGTGGCATGTGCCTGTAATCCCAGCTACTTCGGACGCTGAGGCAAAAGAATCGCTTGAACCTGGGAGGCGGAGGTTGTAGTGAGCTG
AGATTGCGCCATTGCACTCCAGCCTGGGTGACAGCAAGACTCTGTCTCAAAAAAAAAAAAAAAAAAAAGAAACTGAGGGCGATGCCCAGCATTCTAAG
TGATCCTGATTCAAACTCCAATTTGAGAACCTCTGCACTAGACCTCTGTAGACCTCTGGTTCTCAAACTCAGCCGCACATTAGAATTACCTGGGGGCC
TGGAGCGTACTCCAGGGATTCTAATTTTCTGATTTAATTGGTATGGGGTATGGCCTGGGCAAGGGAGTTTTGAAAGTGCCCAGGTAGTTCTATTATGC
TGCAAAGTGTAAAGAGCACTGCCATAGAGCTTTGTTCTTCAGGGGGTGGTCAGTGGGCCAGCAGCATCAGCTCACCTGGGAGTTTGTTAGACTAGGAC
TGTCAGTCTCACCAAGCCCCGCTGAAGCAAATTTGCATTTGACAAGATCTCCAGGTGATTCCTATGCTCTTTGAAGTTTGAAAAGCACTACCCAGGAG
AGGATAGAGGTTATCAACTTCAAGCCTACTCTTGTCCAACAGTTGAGTGAAAAGAGTCAGAGCTGACCTCTCAAGCAATCCTCAGCATCATCTGCCTT
TCAGCCATCAGAGGCAACAGGTGATTTGTTTTGAGCTTGCTGCCCCAGCAAAATGCTCCCTTAATGACCACAAATGAACACATACATTCATTTGTCTC
AGCCAGAAAGGGAGTGGAGAGTGTTACAGCTTATGTCTTTTTAAAGCTACAATTCTTAGCTTCAAAACAAAATTACTTTTATATATATATATACACAT
ATATATCTATGATATATATCATATATGATATATGAGATATATGATATATCTCATATGATATATATATGAGAGAGAGACAAAGTCTCACTCTGTCAACC
CAGGCTGGAGTGCACTGGTGTGATCATAGCTCACTATAACCTCGAACTCTTGGCCTCAAGTGGTCCTCCTGTTTCAGCCACCCAAAGCACTGCGATAA
CAGGAATGAGCCACTGTGCCCAGCCCAAAAATTACCTTTAATTTACTCAAATGTTTGAAAAGTATACCGGACAGAATTCTTAGGGAGCGGTAACGTTA
TACCTATGAAATGTTTAGCATTCATGTATTTCACCTGATAACAGTCCATTGCTGTTTTTATCAGCAGTTAGTCTTTCAAGCAGCCAGAGTTCAGGGGG
AACAATGCTCCCAGTCCCACTGGCTTGCCAGGCCAGCGCTCTAATGGGGAGAGCCCTAGTGAGAATGGAGCTGCGCATACCCTATATCAGAACCACTT
CTCTATTTTAGACCAGTGGCTGAGACACAGGGGTAGGACACAATCAGGCAGGAAGGGGCTGGAGAAGAATTTTGAGCCAAACACACACCAAAGCACCA
AAGACTGTGACCACTTTATTTCATTGTTACAGCAAACTCAGGTCTATCCCCTATTGAAATGAGTCTTCCCCATGTTCATTCTGGTATGCTCTATGGTG
CGGGAGAGAAAGAGGAGGCCTGTGGCCAGGCGAGGACACGCACGGCCCTGTGAACGGCCACGGCAATTTGTGCTGTGCACGTGAGGCCAGAGCTCACG
TGGCAAGTGCGAGAAATACAAGGACCGTTGGCTGAGATTGAGCTACGGTGGCAGCTTTTGTCCACTGACAGATAAGGCCAGAGGTCCTGTGGCCCTTC
AGGGACTGTTCCAGTGAATGATTTTTGGACAGTGGTCAAATCATTTTGAAAAAACTAAAATTGGATACCTACTTTGCAGCTTACAAAGAAATAGATCC
CTGCTTTACAAGTTACACAAAAATAGGTCCCAGATGGATTACAGATCTGTAATAGACAGGATCTGAAATGACATGTGAGAGAATATTTTTGTAATCTT
CTAGATTGGACTCAAAACAGAAGTCTTTCTTTCTTTCTTTCTTTCTTTTTTTTTTTTTTTTTTGACATGGAGTCTCACCCTGTCGCCCACGCTGGAGT
GCAGTGGCGCCATCTCGGCACTGCAACCTCCACCTCCAGGGTTCAAGTGATTCTCCTGCCTCAGCCTCCCCAGTAGGTGCCACTACAGGCACCAACAA
CCACTCCCGGCTAATTTTTCTATTTTTAGAAGAGATGGGGTTTCACCATGTTGGTCAGGCTGGTCTCAATCTCCTGAACTCAGCAAATTCGCCCGCCT
CTGCCTCCCAAGGTGCAGGGATTACAGGCGTGAGCCACTGCACCCAGCCAAAACAGAAGTGTTTCATATAAAAAACGATACTGTGAATCAAAAGACAG
CATACACATTAGGAGGAAATATTTGCATCCAAGTCTAATGTAGCAGATCATGAGTCAAAATCCTTGATCTACAAGAGCCTCTGCAAATTCATAAGAAA
AGTCAACCAAATAGAATAAAAGGTAAAAGATATAAACAATTTAGAAATGAAGAACTAAAAATTATGAAAATATGTTCTGGCCAGGTGTGGTGGCTCAC
GCCTGTTATCCCAGCACTTTGGGAGGCCAAGGTGGGTGGATCACGAGGTCAGGAGTTCAAGACCAGCCTGGCCAACATGGTGAAACCCCATCTCTACG
AAAAATACAAAAAAAATTAGCCGGGCATGTCGTGCTGTGCACCTGTAATCCCAGCTACTGGCGAGGCTAAGTTAGCAGAATTGCTTGAACCCAGGAGA
CAGAGATCACGCCACTGCACTCCAGCCTGGGTGACAGAGCAAGACTCCGTCTCCAAAAAAAAAATTTTTTTCTTTTTAATTAGCCAGGCATGGTGGCA
CACACCTATAGTCCCAACTACTTGGGGGCTGAGGTGGGAGGATCACTTGAGCCTGGGAGGTCAAGACTTAAGTGAGCCAAGATCCCACCACTGCACTC
CAGCCTGGGAGACAGTGATATACTGTCTCAAAAAACAACAACAGGCCAGTCGTGGTGGCTCACACTGTCATCCCAGCACTTTGGGAGGCTGAGGTGGG
TGGATCACCTGAGGTCAGGAGTTTGAGACCAACCTGGCCAACGTGGTGAAACCTCATCTCTACTAAAAATAAAAAAATTAGCTGGGAGTGATGGCAGG
CGCCTGTAATCCCAGCTACTCAGGAGGCTCAGGCAGGAGATTCGCTTGAACCTGGGAGGCAGAGGTTGCCAAGATCGCGCAACTGCACTCCAGCCTAG
GCGACAGAGCGAGACTGTCTCAAAAAACACAAAACAAAACAGAAAAGCAAGAACAACAAAAAAGTGGATGTCTGGGCCATCCAGCCTCTTACTCATAA
GCCTGCTTCCTTAGAAGCCGCCTCCCTACATCTACTCATATCCACAGAATGTGCTCCTCTTGGCATCCTGGAGTAGCCAAAGCCATTGCTATCAGTAA
AAAAATTTTTTTTTAATTCCCAGCTGCCACCATCTCCTCTCATCTCCATTCACAACGTGGTAAGAATATTATTTTTGTATCTGACCAAAACCAGTTTC
TGAGTTTTCATTTTCTTAATTGGCCACCAATAAATAAAGGAGGGACTCACCTGCCCTTGAACGTGCTCTGCTGTGTGCTCTGGATCTCATAGGGGCCA
GCCTTTCTCACGGAGCCTGGAGGGGGCCAGGGATCCCTTATTCTTCCCAGCTCAGTGACTTTTTCCCATTTTGCAGGCCCTTGTCAGTCACATAACCT
GAAGGGCCACTCTTTTCTGTCCTGTAGAATGACTCCTGTGTCTCTCTGGTCTTAGAGGGCAACTTGTGAAGAACTGCCAACCTTCCTTTGGTCTTCGC
TGGATAGCAGCGGTGTTTGTAAAGATAGGACTCGTCTTTGAGCAGTACAGTGAAGGGGTTATTGGGCCTGGATTCTACTCAGAGGGACCTCCTTAGTG
AGGTTTATGATATTTATGTTATACAGCAATGTAGAAATGACCTTCTAAAGGCCAGGAGCGGTAGCTCATGCCTGTAATCCGAGCACTTTGGGAGGCCG
AGGCAGGTGGATCACGAGGTCAGGAGATCGAGACTATCCTGGCTAACACGGTGAAACCCTGTCTCTACTAAAAATACAAAAAAATTAGCCGGACGTGG
TGTCCGGCGCCTGTAGTCCCAGCTACTCGGGAGGCTGAGGCAGGAGAATGGCGTGAACCCGGTAGGCGGAGCTTGCAGTGAGCCGAGATCATGCCACT
GTACTCCGGCCTGGAAGACAGAGTGAGACTGCGTCTCAAAAGAAAAAAAAAAGGGGGAAATGACATTGTAACCAAAATTGGGGTTGGGAGGAATAAGG
TGGTTTTATAGGATATGTTCTTTGATTGTCTTGTATGTAATACCTGGGAGTTCAAAGACTTGACATTTAAAAGAAGATAGCCCCATAAATTAAGTATC
GCACTTAAAGGCTGCCAGATTTTAAAGAAATTCTTTCATATGTTCATCTAATAGTTTGATGCTGCTATTTGGGTTTTTTGTTTTAACATTTAAATCTT
AGATCCATTTGGGATTTGTCCGTTTCTGGGTCCAGCTTGAATTTTTCCATTTGAATTGAATTTTTCCAGTTTTCCGTAACTAGTTCTTCCAGCATCAT
TTATTGTATGTTGTGTCTCTTCTCCATGGATTTAAGAGGCCAACCGTATTGTATGCTAAATTTTCATAGTCATTATACAGATTATCTTTTCTCTTCTC
TTGGCCTGTCTATTTCTGTTCCACTTGTATTAAATTCTGAACTTACATATCTATTGAATCTATTTCTGGAGTTTTTATTTTGTTTATTTGGTCTGATT
GTCTATTAATGTTTTAATTATTGAGATTTCATAATGTTTTTGTTTTTGTTTTTGAGACGGAGTCTCCCTCTGTTGCCCAGGCTGGAATGCAGTGGCAC
AATCTCGGTACACTGCAACCTCTGCCTCCTGGGTTCAAGCAATTCTCCTGCCTCAGCCTCCCAAGTAGGTGGGATTACAGGCGCCCGCCACCACACCC
AGCTAATTTTTGTATGGGGTTTCACCATGTTGGCCAGGGTAGTCTAGAACTCCTGACCTCAGGTGATTCACTCGCCTCGGCCTCCCAAAGTTTTGGGA
TTACAGGCGTGAGCCACTGCACCCGGCCGAGATTTCATAATGTTTTAACATCTGACAGAGATAGTCCCTCTCCCCTCCAATTTTAGTCAGTAATCTTT
CTTTTTTTTTTTTTTGAGATAGAATCTCAGTCTGTCACCCAGGCTGGAGTGCAGTGGCACGATCTCTGCTCACTGCAACCTCCACCTCCTGGGTTCAA
CGATTCAACGATTCTCCTGCCTTAGCCTCCCAACTAGCTGCGATTACAGGAATGTACCACCATGCCCAGCTCATTTTTGTATTTTTAGTAGAGATGGG
GTTTCACCATGTTGGCCAGGCTGGTCTTGAACTCCTGAGCTTGCGATCCGCCCACCTCTGCCTCCCAAAGTGCTGAGATTACAGGTGTGAGCCACTGC
TCTTGGCCTCTTTTTTTTTTTTTTTTTTTTTTTTTCAGAGACAGAGTCTCACTCTGTCACCCAGGCTGGAATGAAGTAGCATGATCATAGTTTACTAT
AATCTCAAACTCCTGGCCTCAAGCAATCCTCCCACCTCAGGTTCCTGAGTAGCTGGGACTACAGGCAAGTGCCAACACACCCGGCTAATTTTGTTTTT
TTTTGTAGAGATGAGGTCTTGGCTGTGTTGCTCAGGCTGGTCTTGAACTCCTGGCCTCATGCAGTCCTCTCACCTCAGCCATCCAAAGTGCTGGGATT
GCAAGCAATAATCTTATCTTTTTTAGTGTTTATTTTTGCAAACCTCTACTTAGCTGCATGCTTTACCAGTTTTAAATGTGAATTCTTTGAACTCCCAG
GTTATTACAGATGAGGCAATCAACGAACATATCCTATAAACCACCTTATTTCTCCCAACCCCAGTTTTGGTTAGAATGTCATTTCTACACTGCCGCAT
AACATTCACAAGCTCTTTAATCACCCCAGTTGCCATGGTAGTTTTAGTCTTCATTCTAAAGTTAAATTTATTAGGTGTTCACCCCTCTAGCAGTCCAT
TAATTTGTGTATGTTCAAAGCTGTTTGCCTATAGGTTTATACTTGAAGGACAGTTTTGTCGAATATAGAGAAATCTTTTTTTTTTCCTGAGATAGAGT
CGCCCAGGCTGGAGTGCACTGGCGCGATCTCAGCTCACCACGACCTCCACCTCCTGGGTTCAAGCAGTTCTCCTGCCTCAGCCTCCGGAGTAGTTGGG
ATTACAGTCACGTGCCACCACACCCAGCCTGAATATAGAGAAATCTGAAACCAGTTGATTTTCTTTCCCCTTGTAAGTGATTTGATCCTTTTGCTGTT
GTCCACTGGTCTTACTGTTAGCCACCCTGGGTTAGTTTTTGTTGGCCCATGGTGTAACTTTCACATGTTCTTATATCCTTACAGGTTTTATCTTTAAG
TATTAGTTCTTTTTTTTTTTGAGACGCAGTCTCACTGTGTCGCCCAGGCTGGAGTGCAGTGGTGTGATCTCAGCTCACTGTAACCTCCGCCTCCCGGG
TTCAAGCGATTCTCCTGCCTCAGCCTCCTGGGTAGCTGCGACTACAGGCATCTGTCACCACACCCAGCCAATTTTTGTATTTTTTTAGTAGAGACGGG
GTTTCACCATATTGGCCAGGCTGATCGTGAACTGCTGACCTTGTGATCCGCCTGCCTCAGCCCCCCAAAGTCCTGGGATTACAGGCGTGAGCCACCAC
GCCTGGCCTTTAAGTACTAGTTCTATTGCTTTGTTTTTTTGAGAACTCCAGTTATGTTTACTGATTCTCCTTTTGCCTAACTTCTGTTTCTATCTTTT
TGTTTAAGATAGGGTCTCACTCTGTCACCCGGGCTGGAGTCCAGTGGTGCAAATATGGCTCACTGCAGCCTCTGCCTCCTGGCCTCAAGCAATCGTCC
TGCCTTGGCCTCTTAAGTAGCTGGGTCTACAAGTGTGTACAGCCACACCTGGCTAATTTTTGTATTTTTTGTAGAGATGGGGTGTTGCCATGTTTCCT
AAGTTGTCCCAAACTGCTGGGCTCAAGCAATCCACCTGTCTTGGCCTCCCAAAGTGCTGGGATTACAGACCTGAGCCAACGCGCCTGGCCCTCTGTCA
TTCTTTTTAATCCTTTTAACCTATTCCTTTGTTTCCATTTCCTGTCATTGCTTTCTTATTTTGGTCCTCTTTTTCCCATTCTTGGAATGGTGCTTTCC
AAGATGCCTATTCCCATTGCGCTCCTTTTCCTGTTGTCTTCATTTCTCTGGCTGATTTTCCCTCCTTTCCTGAGTTCTTCTAGTGTACATTTAATCTC
TTCCTGTTGTCTCACCATCCCTTCTTCAAGCTCTTCTCTGTGGTATTCCTTTATAAAGGCAGTTGCCTCATTTAATTATTTTTATGGATGGAAATGAT
CACTTTTCTCAGTAATAGTAATTCCTTGGGCCGGGCTCCAGCCTATAATCCCAACACTTTGGGAGGCCGAGGAAGTTGGATCATTTGAGGTCAGGAGT
TCGAGACCAGCCTGGCCAACATGGCGACACCCCATCTCTATTAAAAATACAAAAACAATGAGCCCCGGGTGCTGGTGTGCACCTGTAATTCCAGCTTG
TCAGGAGGCTGAGGCAGGAGAATCGCTTGAACCTGGGAGGCAGAGGTTGCAGTGAGCCAAGATAGTGCCACTGCACTCCAGCCTGGGTGACAGAGTGA
GACTTCATCTCAAAAAAATAAATAAATAAATACCTTGCATTAATGTGCGTGGGTCAAAGGCTCTTTCCTTCTCTGCTTTCAACAAACAGCTTCCTGCG
TACATGGCTGCCCTGTGTGATTCCAGTTGCAGCCTCACCTCCTTTGCCTCTCTGAACCAAAGAAGCCAGCCCTGCTTACCCCAGTTCCCAAACACAGG
AAGTGATTTTCTGCCTGAGCTTTCTGAGTTCTGTTCCCTCCCACCCCAGGGCTCTCCATGCTTATTCATTGCATTTCCTTCCTCATTGCTTTTACCCA
GTCTGCTGTTTTGGGAAGCCCTGACATGTATTTTGGTGCCTACATATTTTATCTTCTGATCTCACTGAAAATAAAATTGGATTTTACTTGTTTTCCTT
GTTGCTTCCGAGCTCATTGGAGAGCTAACCAGTTCTGATTTTAAAACAGCAAAAACAGCCTTATTAGCTAATGTTACCTATTCAATTACTAAAATCAC
CCAAAAAAATGTTTTTCGTCATCTTAGTTTGCTAAAAATTAATCAGGCTCTTGCCCTGCAGGTTCTTCTAGAAGAGAGATGCTGGCAGGCCTCTGTGC
TCAAACTTGCCAAAAAACTGTCTTCCGGCCAGACATGGCAGCTCACGCCTATAATCCCAGCACTCTAGGAGGCGGAAAGTGGGCAAATCACCTGAGGT
TGGAAGTTCGAGACCTGCCTGACCAACATGGAGAAACCCTGTCTCTACTAAAAATACAAAATTAGCCAGCCATGGTGGCACATGCCTGTAATCCCAGT
TAATTGGGAGGCTGAAGCAGGAGAATTGCTTGAACCCGGGGAACAGAGGTTGCGGGGAGCCAGGATCGCGCCACTGCACTCCAGCCTGGGCAACAAGA
GCGAAACTCTGTCTCAAAAAAGAAAAAAAAAAAAACTGTCTTCCATGGCCAGGCGCAGTGGCTCACGCCTCTAATCCCAGCACTTTGGGAGGCCAAGG
CGGGCGGCTCACTTGAGGTCAGGAGTTTGAGACCAGCCTGGCCAACGTGGTGAAACCCTATCTCTACTAAAAATACAAAAATTAGCCAGGCGTGTTGG
CGTGCGCCTGTAGTACCAGCTACTCAGGAGGCTGAGGCAGGAGAATTGCTTGAACCTGGGAGGCGGAGCTTGCCCTCAGCCAGGATCGCGCCACTGCA
CTCCAGCCTGGGTGACAGAGTGAGACTCCAAAAAAAAAAAGACTGTCTTGCAGGAAGTTGAGGAAACACAGAATTCCTGGTTCTGCCGAGTCAGAGTA
TACCTTGTGCCCCTTGTGCCTGTATCTGTCCCCTAGTGATATGAGGGGTGGGGCTGGAGCGCAATTCTGTGAACCCACAGATTACCAGCAGCATGTGT
CAGTGTAAGCATCGAGTTTAATTATATGCCTGTTTTCAACTTTAAAGGTGATCTTATGGCCAGGCATGGTGGCTTATGCCTATCATCCCAGCACTTTG
GGAGGCCAAGGCAGGAGGATCACTCAAGCCCAGGACTTTGAGACTAGCCTGGGCAACATAGCAAAACCCCATCTCTACAAAAATACAAAAAAATTAGC
TGGGTGTGGTGGCATACACCTGTGTAATCCCAGCTACCTGCAAGGCTAAGGTGGGAGCGTCACCTGAGCCCAGGAAGTTGAGGCTACAGTGAGCTGCG
ATCTCGCTACTGTACTACCGCCTCGGTGACAGACTAAGACCTTGTCTCAAAAAAATAAAAAATAAAAATAAATGTGATCTTAAATTGACTGAAGGCAA
GAGTGGAGCCCAGGTCCTGCCTGAGAGGGGCATGTCCAATTTTATCATGTTCATATGTAAGAGTTTATATTAACTTACAGAAAATTAATTGAGCATCT
AACCTTAAATAAAGTTTTTTAGCTTTAATAATAGAAATGTCACTAGGAGTTCGAGCGCGATAGCTCACACCTGTAATCCCAGCACTATAAGAGGCCCA
GGCGGGCGGATCACCTGAGGTCAGGAGTTTGAGACCAGCCTGGCCAACATAGTGAAACCCCATCTCTACTAAAAATACAAAAATTAGCTGGGCGTGGT
GGTGTCCCCCTCTAGTCCCACCTACTCTGGAGGCTGACGCAGGAGAATCTCTTGAACTCGGGAGGCTGAAATTGCAGTGAGCCTAGATCTCGCCACTG
CAGTCCAGGCTGGGTGACCAAGTGAGACTCTGTCTTAAAAAATAGAAAAAAGTGCAGCTTTTTTTAGGATTGAGGTCAAGAGGGAAATTTCCTCCTCA
GGCTCATCAAGAGGACATTGTGTGGTATAAACAATGTCATCAACAACTTCCTGGCAGCATTAATGTTCCCAACAGCCATTAATTTTTTTTTTTTTTTT
CGAGACAGAGTCTTGGCTCTGTTGCCCAGGCCGGAGTGCAGTGGCGTGATCTTGGCTCACCAAAGCGTCCACCTCCTGGGTTCTAGTGATTCTCCTGC
CTCAGCCTCCCAAGTAGCTGGGATTACAGGTCCCTACCACCATGCCCAGCTAATTTTTTTGTATTTTTATTAGAGACGGGGTTTCACCATGTTGGTCA
GGCTGGTCTCAAACTCCTGATCTCAGATAATCTACCCACCTTGGCCTCCCAAAGTGCTAGGATTACAGGCGTGAGCCACCATGCCCGGTGTCAGTCAT
TAATTTCTAATGACACTTCTTAGAGTGTCAGCCAGGGCTATGAAGCCAACACACTATTCAGTAAACACAGTTCTGCCGGGGGTCAGAACAGTGTGATC
CAGGCTCAGCTCTCTGCTGCTTTGGCCTCATCCTCGGGTAGATTTTACAGTCCCTCATCTAAGGAAGGCGCTATGTTCTTCTAGCACTCCTCCCTAAT
GATTTCTTTTACTCAAATATTTGGGCACTGGAAGGTTTAACAAGTTCCTGGAAGACATATTACCAATTAAAGATCTATTCATGGCTGGGCGCGGTGGC
TCACGCCTCTAATCCCAGCACTTTGGGAGGCCGAGGCGGGCGGATCACAAGGTCAGGAGATCGAGACCATCCTGGCTAACATGGTGAAACCCCGTCTC
TCTAAAAATACAAAAAATTAGCCGGGTGTGGTGGTGGGCGCCTGTAGTCCCAGCTACTCGGGAGGCTGAGGCAAGAGAATGGCGTGAACTGGGGAGGC
GGAGCTTGCAGTGAGCCGAGATCGCGCCATTGCACTCCAGCCTGGGCCACACAGCGAGACTCCGTCTCAAAAAAAAAAAAAAAATCTATTCATAAGCT
CCCACTGCACACAAATTTGAGGGCTCTTTTAACAATCCAGTTAACTTGCTTCTAGTCATTGGTATACACACCTAGGGTCCTTATTGCACGGGCTCTGT
CATCTTTCCTGTGTCTAAATACAAAGTAATGCGGTCATAAACACCAGATACATGTTAATACAAGAAATTATTATATAAGCTCCTTTTGAAAGGAGACT
GAGCTTTAGGGAAATGTCTATGGTACCAATAAAAAAACTCATTCATGCACTCATTACATTGAATCAAGCACCAGGCATTATGGTGAGTGTGGGGGTAA
TTGGGTAGGTACCCTGTTTATGTCCTTTAAAATTCCCACCTGACCTCATTTTCAGATGAAGACACAGGTAGTGGGGACATGGAACCCAAATCTTTAAG
GCTTTGAAATTCTGGGTGATTGCCACAAAGATTATGAGGCCTGACAACCAGTCAGAATGAAATAAGCCTGGGTTGGTTTTTTTTTTTTTTTTTTTCCA
TCAGCTTTTAGGTTCAGGGGTACATGTGCAGGATGTGCAGGTTTGCTACAAAGGTAAATGCGTGCCATGGTGGTTTACTGCACAGGTCATCCCACCAC
CTAGGTATTAAGCCCAGCATCCATTAGCTATTCTTCCTGATGCTCTCCCTCCCCCTCCCCCACACGCCCCAGGGTGTGTTATTTCCCTCAATGTGCTC
ATGTGTTCTCTTCATTCAGCTCCTGCTTATAAGAGAAAAGATGCAGTGTTTGGTTTTCTGTTCCTATGTTAGTTTGCTGAGGATAATGGCTTCCAATT
CCATTCATGTCCCTGCAAAAGATATGATCTCATTCTTTTTTTTCGCTGCATAGTATACCATCCTGTATATGTACTACATTTTCTTCATCCAATCTATC
ATTGATGGGCATTTGGGTTGATTCTATGTCTTTGCTATTGTGAATAGTGCAGCATTGAACATGTGTGTCCATGTATCTTTATAATAGAATGATTTATA
TTCCTTTGGGTATATTAAACCTGATTATTAAAGGCTTTGAGGATTTTCTAGCCTCTTTGTGTTCACTGGAAGTTTAATTTTTATAAATTTCCCCCTTC
GTTTTCTTTTGTCATCATGACAGGGAGTTGAGTAGACATTGTATGTTTTTCTTCATTGTTTCAGATTTTGAGATACAGAGTGAAAATGGGGAGAACTG
TAATCAAGACATGTTTGAGAATGAATCACGTAAGATATTCTCGGAAATGCCTGAAGGTGAAAGTGCTCAGCACTCCGATGGGGAAAGTGACTTTGAGA
GAGATGCTGGCATCCAGAGGCTCCAGGCACACACCCCAGGTGAGGACCACGGGCAGGTGGTTTCTCAGGACAGGGAAGTTGGCCAGCTAATAGGCCTG
CAGGGCACCTACCTAGGGGAGAAGCCCTACGAATGTCCCCAGTGTGGGAAGACCTTCAGCCGGAAATCCCACCTCATCACACACGAGAGGACCCACAC
AGGAGAGAAATACTACAAATGTGATGAATGTGGAAAAAGCTTTAGTGATGGTTCAAATTTTAGTAGACACCAAACCACTCACACCGGGGAGAAGCCCT
ACAAATGCAGAGACTGTCGGAAGAGCTTTAGCCGGAGTGCCAACCTCATAACCCACCAGAGGATCAACACGGGGGAAAAGCCCTTCAAGTGTGCCGAG
TGTGGCAAGAGCTTCAGCAGGAGTCCCAACCTCATTGCACATCAGCGCACCCACACAGGAGAGAAACCCTACTCGTGCCCCGAGTGTGGAAAGAGCTT
TGGCAACCGATCCAGCCTTAACACGCATCAGGGGATCCACACTGGAGAAAAGCCCTACGAATGTAAAGAATGCGGCGAAAGCTTTAGTTACAACTCCA
ATCTAATCAGACACCAGAGAATCCACACAGGAGAGAAACCCTACAAATGTACCGACTGTGGGAAGAGGTTCAGCCAGAGTTCAGCCCTCATCACCCAC
CGGAGAACCCACACAGGAGAGAAACCCTACCAGTGCAGCGAGTGTGGGAAAAGCTTCAGCCGCAGCTCTAACCTGGCCACACACCGGAGAACCAACAT
GGTGGAGAAGCCCTATAAGTGTGGGGTGTGTGGGAAGAGCTTCAGCCAGAGCTCCAGTCTGATTGCACACCAGGGCATGCACACAGGGGAGAAACCCT
ACGAGTGCCTGACATGTGGGGAGAGCTTCAGCTGGAGCTCCAACCTCCTCAAGCACCAGAGGATCCACACGGGAGAGAAACCCTACAAATGCAGCGAG
TGTGGGAAATGCTTCAGCCAGCGCTCCCAGCTCGTAGTGCACCAGCGGACCCACACGGGCGAGAAGCCCTACAAATGCCTCATGTGCGGCAAGAGCTT
CAGCCGGGGCTCCATTCTGGTCATGCACCAGAGAGCCCATTTGGGAGACAAGCCCTACAGGTGCCCTGAGTGTGGGAAAGGCTTTAGCTGGAACTCAG
TCCTCATTATACATCAGCGAATCCACACTGGGGAGAAGCCCTACAAATGCCCCGAGTGTGGCAAAGGCTTCAGCAACAGCTCTAACTTTATCACACAT
CAGAGAACTCACATGAAAGAGAAACTTTATTGAAGTGGCAAAGAGTGAAAGTGAGGGACTGGCCTGGAGTGGGAGTTGCCACACTGCCCCAACAGTGA
TTCCCTTTCAAAGAGCTGTGCTTCCTAAACATTCTGGGGGGTTTTGCCAGAGTCTTCCCCTTGCTCATCCTCATTTCCAGGACACTGTCATTTTAGTG
GTCTGAGTCAAGTCCCGTATACATTCAAGAACAGGGCATAGGCGTGGAAGGTCTGGAAAGTTGGGTCTTTTTCCCTTACATTGGGTGACTTGATTGGC
CCCCTCTCATGATTCCTCTGTGCCTCAGTTTCCTCTTTGGTAAAATGGGGGGAAATGTTTCTCCATGTGGAATGGAAGACAGCATGGCCCACAACGTG
GGCCGAGTCCTCAGAGAAATACTGGAAATCATTGGTGTGGTTCTGGTTGTTTTGTTGTTTTGCTGCCACGTTGTTGGGCTAAGGTGCCTTCACCCCAA
GCTGTTAGTGTTCCAGGGCACCCCAAGCTGTCAGTTAGAATCTGCTCTTCTGGCTTTGGTGTCTTGGGCTTTGATTTCAGGTCAAGATGGAGGGGCTT
CTCCAGTTCTGAGTCACCCACGTGAAGGTAAAGACCCTTTCTATTTCCAGAAAGTGTCAGGAGCACAGAAACTTGAGGAAGTACAGCCTGGAGCCAGT
GTCCCAGTGTCCTTTCCATTGGTAAGAGTTGGACAGGGCCTTCAGGAAAGGGGTAAACCGAGGACATTTCAGTGCTTGCTTTTGTCTCTGCCTACTGT
CCTGTGGTAGATCAGCTACCAGGGGAACACATTTGTTCTCGTGGGGTTTTGTCCTGGAGAGTGTAGTGAAGTCCGAGAGCCCTAGCTGCCAACCCATG
GTGGATGGTAACTTCTGTCTCATCAAGAGTAAAACAGTCCTGCACACAGCAGGGTGGGTTTGTGCCTTTGGCCCAACAGGTACATAGCCCCATAATTT
CTGAATTATTCTATGCACTTGTTTCCCTCTTCTTTTATTTTTTATTTGATATATGCCGAGCTAGAATCCTGTCGGGTAGCTTTTGTATACTAAGAACA
TTATTATTATTATTATTTTTGAGACGGAGTCTCACTCTGTCACCCAGGCTGGAGTGCAGTGGTGCCATCTCAGCTCACTGCAAGCTCCGCCTCCCGGG
TTCACGCCATTCTCCTGCCTCAGCCTCCCGAGTAGCTGGGACTACAGGTGCCCACACCACCACACCCAGCTAATTTCTTTTTGTATTTTTAGTAGAGA
CGGGGTTTCACCGCGTTAGCCAGGATGGTTTCGATCTCCTGTCCTCGTGATCTGCCCGCCTTGGCCTCCCGAAGTGCTGGGATTACAGGCGTGAGCCA
GCGCACCCGGCCAAGAACATTATTTTTAAAGAAGTGTTAACTTTGAGGACATATCTGTTCCCTGGAGATATTTGGGCTTGAATCAGGAGTTTGTCCTA
CAGGTGTCGCCCTTGATCTCAGGATGCTACCAGGGCTTTGTTCTCGGGATCCTCGCACCTGGAGAGTGAAGACGGGCATGACGGCAGGTGAAGGGGTT
TGCTGTGAAGGAAGAGGAGATAAGGCATTTCCAGGAAATGGGAAACTGCCTCCTCCTACACATGGGGCCTGTGCTCAGAATGGGCTTAGTTCTTATAG
GATGGATGCTCAGTATTCCTTAATAAAGTAGAGTTCCATTCTTTTCCTGAGTCTGTCTTTTACTGTGTTAAAAACCTGAACTAGGCTGGGCGTGGTGG
CTCACACCTGTA
HUMAN SEQUENCE—mRNA
CGGGACTACTTGTTGATATTTGAGGAGGGAAGTGTCTTACCTGAGAGCCTGGCTGGAGAAGACTGAGGTCCAAGGCTTGAAGCCTAAGTGATTGCCCC
AGGACTGTGGATGATGGCTGCAGACATCCCGAGAGTGACCACTCCGCTGAGCTCCTTGGTCCAGGTGCCTCAAGAGGAAGATAGACAGGAGGAGGAGG
TCACCACCATGATCCTGGAGGATGACTCCTGGGTGCAAGAAGCTGTGCTGCAGGAGGATGGCCCTGAGTCTGAGCCCTTTCCCCAGAGTGCTGGCAAG
GGCGGCCCCCAGGAGGAGGTGACCAGGGGACCACAGGGTGCACTCGGCCGCCTCCGAGAGCTCTGCCGGCGCTGGCTGAGACCAGAGGTACACACCAA
GGAGCAGATGTTAACCATGCTGCCAAAGGAAATTCAGGCTTGGCTGCAAGAGCATCGGCCTGAAAGCAGTGAGGAGGCAGCGGCCCTGGTGGAAGACT
TGACCCAGACCCTTCAGGACAGTGATTTTGAGATACAGAGTGAAAATGGGGAGAACTGTAATCAAGACATGTTTGAGAATGAATCACGTAAGATATTC
TCGGAAATGCCTGAAGGTGAAAGTGCTCAGCACTCCGATGGGGAAAGTGACTTTGAGAGAGATGCTGGCATCCAGAGGCTCCAGGGACACAGCCCAGG
TGAGGACCACGGGGAGGTGGTTTCTCAGGACAGGGAAGTTGGCCAGCTCATAGGCCTGCAGGGCACCTACTTAGGGGAGAAGCCCTACGAATGTCCCC
AGTGTGGGAAGACCTTCAGCCGGAAATCCCACCTCATCACACACGAGAGGACCCACACAGGAGAGAAATACTACAAATGTGATGAATGTGGAAAAAGC
TTTAGTGATGGTTCAAATTTTAGTAGACACCAAACCACTCACACCGGGGAGAAGCCCTACAAATGCAGAGACTGTGGGAAGAGCTTTAGCCGGAGTGC
CAACCTCATAACCCACCAGAGGATCCACACGGGGGAAAAGCCCTTCCAGTGTGCCGAGTGTGGCAAGAGCTTCAGCAGGAGTCCCAACCTCATTGCAC
ATCAGCGCACCCACACAGGAGAGAAACCCTACTCGTGCCCCGAGTGTGGAAAGAGCTTTGGCAACCGATCCAGCCTTAACACGCATCAGGGGATCCAC
ACTGGAGAAAAGCCCTACGAATGTAAAGAATGCGGCGAAAGCTTTAGTTACAACTCCAATCTAATCAGACACCAGAGAATCCACACAGGAGAGAAACC
CTACAAATGTACCGACTGTGGGCAGAGGTTCAGCCAGAGTTCAGCCCTCATCACCCACCGGAGAACCCACACAGGAGAGAAACCCTACCAGTGCAGCG
AGTGTGGGAAAAGCTTCAGCCGCAGCTCTAACCTGGCCACACACCGGAGAACCCACATGGTGGAGAAGCCCTATAAGTGTGGGGTGTGTGGGAAGAGC
TTCAGCCAGAGCTCCAGTCTGATTGCACACCAGGGCATGCACACAGGGGAGAAACCCTACGAGTGCCTGACATGTGGGGAGAGCTTCAGCTGGAGCTC
CAACCTCCTCAAGCACCAGAGGATCCACACGGGAGAGAAACCCTACAAATGCAGCGAGTGTGGGAAATGCTTCAGCCAGCGCTCCCAGCTCGTAGTGC
ACCAGCGGACCCACACGGGCGAGAAGCCCTACAAATGCCTCATGTGCGGCAAGAGCTTCAGCCGGGGCTCCATTCTGGTCATGCACCAGAGAGCCCAT
TTGGGAGACAAGCCCTACAGGTGCCCTGAGTGTGGGAAAGGCTTTAGCTGGAACTCAGTCCTCATTATACATCAGCGAATCCACACTGGGGAGAAGCC
CTACAAATGCCCCGAGTGTGGCAAAGGCTTCAGCAACAGCTCTAACTTTATCACACATCAGAGAACTCACATGAAAGAGAAACTTTATTGAAAAGTGT
CAGGAGCACAGAAACTTGAGGAAGTACAGCCTGGAGCCAGTGTCCCAGTGTCCTTTCCATTGGTGTCGCCCTTGATCTCAGGATGCTACCAGGGCTTT
GTTCTCGGGATCCTCGCACCTGGAGAGTGAAGACGGGCATGACGGCAGGTGAAGGGGTTTGCTGTGAAGGAAGAGGAGATAAGGCATTTCCAGGAAAT
GGGAAACTGCCTCCTCCTACACATGGGGCCTGTGCTCAGAATGGGCTTAGTTCTTATAGGATGGATGCTCAGTATTCCTTAATAAAGTAGAGTTCCAT
TCTTTTCCTGA
HUMAN SEQUENCE—CODING
ATGATGGCTGCAGACATCCCGAGAGTGACCACTCCGCTGAGCTCCTTGGTCCAGGTGCCTCAAGAGGAAGATAGACAGGAGGAGGAGGTCACCACCAT
GATCCTGGAGGATGACTCCTGGGTGCAAGAAGCTGTGCTGCAGGAGGATGGCCCTGAGTCTGAGCCCTTTCCCCAGAGTGCTGGCAAGGGCGGCCCCC
AGGAGGAGGTGACCAGGGGACCACAGGGTGCACTCGGCCGCCTCCGAGAGCTCTGCCGGCGCTGGCTGAGACCAGAGGTACACACCAAGGAGCAGATG
TTAACCATGCTGCCAAAGGAAATTCAGGCTTGGCTGCAAGAGCATCGGCCTGAAAGCAGTGAGGAGGCAGCGGCCCTGGTGGAAGACTTGACCCAGAC
CCTTCAGGACAGTGATTTTGAGATACAGAGTGAAAATGGGGAGAACTGTAATCAAGACATGTTTGAGAATGAATCACGTAAGATATTCTCGGAAATGC
CTGAAGGTGAAAGTGCTCAGCACTCCGATGGGGAAAGTGACTTTGAGAGAGATGCTGGCATCCAGAGGCTCCAGGGACACAGCCCAGGTGAGGACCAC
GGGGAGGTGGTTTCTCAGGACAGGGAAGTTGGCCAGCTCATAGGCCTGCAGGGCACCTACCTAGGGGAGAAGCCCTACGAATGTCCCCAGTGTGGGAA
GACCTTCAGCCGGAAATCCCACCTCATCACACACGAGAGGACCCACACAGGAGAGAAATACTACAAATGTGATGAATGTGGAAAAAGCTTTAGTGATG
GTTCAAATTTTAGTAGACACCAAACCACTCACACCGGGGAGAAGCCCTACAAATGCAGAGACTGTGGGAAGAGCTTTAGCCGGAGTGCCAACCTCATA
ACCCACCAGAGGATCCACACGGGGGAAAAGCCCTTCCAGTGTGCCGAGTGTGGCAAGAGCTTCAGCAGGAGTCCCAACCTCATTGCACATCAGCGCAC
CCACACAGGAGAGAAACCCTACTCGTGCCCCGAGTGTGGAAAGAGCTTTGGCAACCGATCCAGCCTTAACACGCATCAGGGGATCCACACTGGAGAAA
AGCCCTACGAATGTAAAGAATGCGGCGAAAGCTTTAGTTACAACTCCAATCTAATCAGACACCAGAGAATCCACACAGGAGAGAAACCCTACAAATGT
ACCGACTGTGGGCAGAGGTTCAGCCAGAGTTCAGCCCTCATCACCCACCGGAGAACCCACACAGGAGAGAAACCCTACCAGTGCAGCGAGTGTGGGAA
AAGCTTCAGCCGCAGCTCTAACCTGGCCACACACCGGAGAACCCACATGGTGGAGAAGCCCTATAAGTGTGGGGTGTGTGGGAAGAGCTTCAGCCAGA
GCTCCAGTCTGATTGCACACCAGGGCATGCACACAGGGGAGAAACCCTACGAGTGCCTGACATGTGGGGAGAGCTTCAGCTGGAGCTCCAACCTCCTC
AAGCACCAGAGGATCCACACGGGAGAGAAACCCTACAAATGCAGCGAGTGTGGGAAATGCTTCAGCCAGCGCTCCCAGCTCGTAGTGCACCAGCGGAC
CCACACGGGCGAGAAGCCCTACAAATGCCTCATGTGCGGCAAGAGCTTCAGCCGGGGCTCCATTCTGGTCATGCACCAGAGAGCCCATTTGGGAGACA
AGCCCTACAGGTGCCCTGAGTGTGGGAAAGGCTTTAGCTGGAACTCAGTCCTCATTATACATCAGCGAATCCACACTGGGGAGAAGCCCTACAAATGC
CCCGAGTGTGGCAAAGCCTTCAGCAACAGCTCTAACTTTATCACACATCAGAGAACTCACATGAAAGAGAAACTTTATTGA

TABLE 5
MOUSE NOMENCLATURE
ICSGNM Kcnj9
Celera mCG4483
HUMAN NOMENCLATURE
HGNC KCNJ9
Celera hCG39735
MOUSE SEQUENCE—GENOMIC
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNCCTCATGAATGCTGAGACTAAAGGTGTGC
ATCACCCCTGCCCAATTTCAAAATAGTGACCCAAGGGAAGACCAGATTACAAGGTGCTGCACTACAAAGTGAGAAAATGTTAACGGTTACCCTTTAAA
AACTTTGCTTAGAGGGAAAAAAAAAACCCCACAATCATAACCAAAGCAATGGACCAGGAACTATTTTCCTGCCTGTTTTGTCTTTTCAAATTTCTGTC
ATCTTCTGCTCCTAGAGAGGAACGGCTACAGTAAGATGGTCTGAAGACCTGGTAGTTTTTTTTTTTTTTTTTTTTTTAAGATTTATTTATTTATTATA
TGTAAGTACATGTAAGTAAGTACATTGTAGCTGTCCTCAGATACTCCAGAAGAGGGCATCAGATTTCGTTACGGATGGTTGTGAGCCACCATGTGGTT
GCTGGGATTTGAACTCGGGACCTTTGGAAAAGCAGTCGGTGCTCTTAACCACTGAGCCATCTCGCCAGCCCAGACCTGGTAGTTTAAGCCTGCAATCT
CAGCTGTTTGGGGAGGGGAAGCAGGAGGGTTGCAAGCTCAAAGCCTGAGCTACAGAATGAGTTCAAAGCCAGTGTGAATAACTTAGCAGGGCTCACAG
TCTTGACATTCAGAGATGGGGAAGATTATGGGGCTGAGCTCAGACCACAATATAAAATGAAGAAGGAACACAGAGGAGAGAAGCCAAGAACTGTCGGG
GTTTATGAAATCATTACAAGACACAAGAATTTATTATTTTTCCAGAATTGTTACCCAAGCATTTGGCATCCATCGCCACCTACATGTCAGTGTCCACC
TGGACAGAAATCTCAAACTTAGTCCAGCGTAGAACATCTTACCCACAGGAGCGCTCCTCATGGGACTATGTCACCATCATCCAACTAGAAACACAGCA
GTCATCTCAGCCTCCTTAGTCTTCCTTACAGCACCAACTCCATCCTCTAACCAAAGCATCTCCCACTCAGCACGCCCTCCTGCCCCCCTCTCTCTCTC
TCCCTTTATCGCTGCTGCAGTCTACAGCAGATGCACCTCTCAGCAGGGATCCTGCAGCAGCAATCTAGTGCCTTATCCCCTCCAGTCTTTCTACACTC
CAATAATGCTTCAGGTCACTAACTCCTTTATGTAAAAACAATTAAGGCTCAGCAAGATGGCTCAGAGGGGTAAAGGCAACTTGCTGCCAAACTTGATG
ACCTGGGGTCAATCTCCAGTACTGATGTGGTAGGAGAGACTCAACTACCAAGAGTTATCCTCTGACCTCTACATGTGTGTTGTGGTACACCCACAAAC
ACAGAGAGAGAGAGAGAGAGAGAGAGAGAGAGAGACAGACAGACAGACAGACAGACAGACAGACAGAGACACACACAGAGAGAAATGTAACGTTTAGA
GAAGAATCCATCCATATTCTTTAGCACAGAACAGAAGGCACATTAATTATAACCTGGGCATCCTGCCCTGTCTTCCTCACATCCAACTCTATAGCTGC
TTCCTCCTCTAACACCCAAGGTTGTTAAGTCTTGTGTCCCCTTCTGTATCTTGCTCCTTGTTCTTTGGTCACACAGTGACCAAAGTCACTGAGTGTTG
TGCAAACCTCTTCTTCTTGACTCCTGTATCTCTCTGGAGCTCTACTTAGGCTCCAGTACCTGCAAGGGATTAATGCCCTCACATGACAGGCCCCAGAC
AGAACCCATCCTCTTTCCCTCTCACCAAGGTTGGGAATGCTCACAGCTCCCTGATTTCTGTGTAACTCCTGTCAAGCAGACTGAAACACCGACATTAC
ATCTTGCTCTTTATGCTTGCCTATGTCCCATTCTGTGTCATGACAATTCAGCCACCAAGTTCTGTTAACTCTCCCTTGGTTATATTTCTCTAGGATAC
ACATTTTCATTTCTATGGCCAGAATCATAAAATTACCACTAGCCCAGGACCTGACCCATCCCTCACCCCTCTTTCCAGTATCAAAGGGAGACAAACTG
TTTTTATTAAAGATGTACTGTATTTAAAAAAACCTAGAATCAAAACTTTGAACAAAGTGGGGTGTGATGGTATACACCTTTAATCCCAGCACTTGGGA
GGCAGAGGCAGGTGGATTTCTGAGTTCAAGGCCAGCCTGGTCTACAAAGTGAGTTCCAGGACAGCCAGGTCTACACAGAGAAATCCTGTCTCGAAAAA
ACCAACCAACCAAATAAATAAATAAATAAATAAATAAATAAATAAATAAATACAAGGTCTCTGGATAAACTCCTTCCAAATAGAAATGAGAAGCCATC
TGGTGAAGCTCAGTGTGAGGGTGAGGTGGCACGAGATGGAACTGGGCAATTGGAAAGAGTTAGGTATTGTAGAGCCACAGGAGAGCAGGACTGTGGTG
ACTTCTGTGGCCCTGTGATGTTCTCACTCAAGAGTGACTTACATCAGGATTCCATTCTTAAATAAGCACACTTTATTAGCAACTATAACTCTGTATAC
ATTGTGTTTGCTTTTAATATTTAACTTTTTGTTTTCCAAAAAGAGTTCCTGAAACATACAACAAGCAGAAATTGTCATTGCTGAAGGATGCTTAGCAT
GCTCATGTTTCTGAGTGTTTACTAGGCGTGATAAATTTGACTTTTCTTGTTTTCTTTCAGTTCACTCTGTCTGATGCTCCTGCCCCGGTCTCCTAAAT
GCAGGGATTATAGGTGTGCACCGCCACACCTAACTGTGTACAGTAGATCGTAAGATGGGAAATCCCAGAGTCAGGGACCTTAGGTGGCTGACCTATAC
ACAGTGACATGCCCAGGAAGTGTTAAATCTGGCATTTGAATCCACCTGTTTGACCCCAGAGTTTGTCAAAGGGTAATAGTACAGCGCTCTTGCATGAC
TTAAAGAGATGCTCATTTTCCCAAGAGAACCAAGAGGTTCTAGTGGCCAAATGTCAGTATGAATAAATCTGCTGAGATGCGCTGTGCAGCGTCCGTCG
ACCTTACAGGAGGACAGAGCAATCCTTTTCCTTTTTGATTCATCGCTCCTTTCAGACTTGATCCTCTCACCACAGATCTCTTTCCTTCCACTTCCTCA
TTCAAAATGGGGTCAGTTCCCCCTCAGAACAAAAGAGGAACATGAGGCGAAGACCCTTTGCAGAGGGAAAATCCACAGCTGGGCGTAGGCCGAGGGAG
CTTTCGCTGGGAGAAGCAGGTGAGTTCGGATGAAGGGAAGCAACTGAGAGAGGCAAGGCAGATCCTCAGACGGGGCGGGTTGGGGGGGGGGCGACTCG
GAGAGGGAGTTTTCGGGGAGTCATCAGAGCTGGCCAGGAAGAACTAGGCATGAACATGAGTCCCAGGGACTCCGAGGGACACATTTCTGCTTAGGTCC
CACAGTATTAACACGGTCCACTAAAAGCAGATACGCTCAGCAGGATGAGCGGCCACAGAGGAGAGCCTATCAGTACTCGGTTTAGTCATTACCTTTTA
ATACACATGATTTATATAAGCCTGTATGTGTATAAGACTTAAGTTATAAATGGCTAATTACATTACAGAAGGACTACAGAAGGCAGAGAGAGGGAGGG
AGGGGAGGGCAAGGGTGGGGAGGGGAGGGAAAGGAAGATGCTCTTTACCTATAAGGTTTATCTAGTATCTTTCTAATTGGTCCTTTTAGTGGCAATTC
TGTTAACATTCAAATACACCATGGAGAGGGAAGAACAGAAAACCCCCAGATGCCTGGAACTGGGGAAGCTGTCTTAACCCTGACCTCTCTTGGGATGC
TCTTCTCATCTATAAACTAATGATTACTTTAGATCACTTCTGAATGACCATGGTTAAGTCCTGGTCTAACTCTATCCAGCCCCGTAGACCTGGTAGAC
AAGATGGACCTGTGCGTAACTCTTCTAGGGCTGATTCCACATGGAATTTACCTACTTTTATTTAGAGATGAGGTCTCACTGTGTCCCTCTGGATGAGC
TGGAACTCACCACACACACCAGGGTGGCCTCAGACTCAGAGATTTACTTGCCAGTGCTTCTCAAATGTTGGGGTAAAAAGCGTAAGCCACCACCCACA
GACCCCATGAATTCATATCAATTGTTATTTGAACTAACTTGACCTTCCTACTCCCCTCAGCTCACATCCTCAACCGTCCCTGCCTTCCCCTCCAGACT
TCCTCCCCCATTTCCACGCTTTTGCTCAAGAAGTCTCATGATCTCGTTCAAGGAAGCTCTCCCAGGTTGGCTGACCTCATAGCTGGCAACAAAGGCAA
CTACTGCTAGGGGTGAACACAAGGCTACAGTGCACTCATCCTGCACCCAAACTCAGAATTGCACCAAAGTGTGTGTGTGTGTGTGTGTGTGTGTGTGT
GTGTGTGTGTGTGTGTGTGTGTGTGTGTCTGTGTCTGTGTCTGTGTCTGTGTCTGTGTGTCTGTGTGTCTCTGTGTGTGTCTGTGTCTGTGTTTGCTT
TCACTGTGTATGCATATGTGGAGACCAGAGACGAATGAGGAGATCATAGACATACACTGTCACACCCAGCCTTTCTGTGGATGCTGGGGATCCAAACT
CAGGTCCCCAGATCCATGCAGCAAGTCCTTTGCCCACTAGGCTGTCTCCCGAGCTCTGCACCTAGGCTCTTTATAGGACCAGCAGTGTGGCCTCACTG
TCCTCTATTTCCAATCTGTGTTTATTACAACTCCGCTGACATATTGGGGTTGATTTCTTGGAGGGATGCTTTTATTCTCTTGGTGAAATATTTTTCTG
TGCACTGATGGCTTGTGAAATTTTCTTCTCTGTTGCCTCAGTTCAAGCCAGACGAACAAGGAGCTGAGATTAAGCTTAGTAAGTAAAGCCCAGGACCT
GGAGGATCTGGAAACTGGGTGAAAGAGTTGTCCTCTGTTGGCTAGGTTAGGTTCAGGGCAGCCAGGATGGAGTCAGAGGGGTGGCTGACAACACCCAG
GGCCCACTGTCAGCTCTGTGACTTTCCCTCCAGAAAAAGGGGCCAGTTCTGTGCAAACATGTTCTTGTCCAGGAGTTTGGTTTCTTCTCTCTGAGCAC
CTGGCACAGTGGCACCAATGTGAGCAGTCACTTGGCAGGGCAGAGAAAAGCAAGCTAGCAGTCCCCAGGCTCAGGTGACAGAGCCAGGCCCAGGAGAC
AGGGATATTGACTGGGGCTTTAACAGCACTATTGATGCCAATCTCGGGCAAAAACCTGATATTTCCACTTGGAATAACAAGAAACAGCCAAGAGGATT
GGACAGAGGTCAGTGGACAAGGAGAGCCCTCTGCAGGTCGTGCTGGGTGATTCCAGAACAGAAGAGGGCAGCCCCTGCTGGACAGGGTCTCCTGAGAT
GATGGTGATGGTGACGGTGATGGTAATAGTGATGGTGACAGGGACAATGACAGTAGTAGTGGCTAGGAGAAGGAAAAAGAAGAAAGAGAAAAACACAA
TGTCAGGCTTTAAATAAATAATCCTCATGAAGTAGATACTATTTATTGTGGTTTTGATATGAAACACCCCTCCCCCAAGGGCTCGGGTATTGGAGATT
TGAGCCCCAGCGTGTGGTGCTATTGAGAGGTGACTTTGTTGATGGCGACTTTGTTTGATGAGGAGATAAGAGGTGGACTGTAAGGAGGTGAAAGCTGT
CTGAGGAAGTAGGTCACCAGTGGTGTGCTCTCAAAGGTGGGTCTCAACCTTCCCAATGCTGTGACGCTTCATTTAACACAGTTCCTTATGTTGTGGTG
GCCCCCCCAATCATAAAATTAGTTTTGTTGCTGCTTCATAACTGTAATTTTACTACTGTTATAAATCATAATGTAAATATTTTTGGAGCTTAGAGGCT
TGCCAAAGGGGTCACGACCCACAGGTTGAGAACCACTGCTCTAGAAGGAAGTCACCTCCTCTTCTCTCCTTGTCATTCTCTTTTCTTCCTCTCCCTTA
CACTCCCCTTCTTCTCTTCTCTCTCCCTACTCCTCACCTCTCCACTCTCCATGAAATCTGATTTCCCCTGCTATCTACCACAATGCCGGATCTCATAT
TCCCAAGAAGAATGGAGACAAACAACCCTGGACTGGATTCCTCCTCTTTTAAGTGTGACTTGGGTGTTCTGTTACAGCAATGAAAAGCTAGCAATATA
AGATGGCTAGTCTCATCTCTTAGATTTAAAAAACTAACATTTTCCAAACATAGTGGCTCATGTCTGTAGACACAGAGCCAGGGAAGCAGAGGCAGAAG
GATCCACTGCAGGTCCAAGCCTGGCCTGCACTATGTAACAAGACAGAGAGAGAGAGAGAGAGAGAGAGTAGAGAGAGAGAGAGGAGAGATTGAAGGAA
AGAAAGATTGAAGAAAAGAAAGATGAAAGAAAGAAAGAAAGAAAGAAAGATTGAAGGAAAGAAAGAGAGATTTAAAGAAAGAAAAAAGAAAGATGAAA
GAAAGAAAAGGGAAGAAAGATTGAAGGAAAGAAAAGAACGGAAGGAAGGAAAGAAGGAAGGAAGGAAGGAGTGCAGGGGGAGGAGGGAAGAAAAGAGT
GGAGGGGGAGGAGGGAGAAAAGAAAGAAAAGGAGACTATATGAAGCTATTTGCTCAAAGCCATGCATCTTCTATCAGAGAGTAGAATTTGAACTCAAG
TCATTGCCTCTGAAGCTTGTATTACCCCACACACCTGTCATAGCTCGTGAGCACATTTCAGAAACTTCTAGTCTTCTATTGTGCTGTTTCTTCCTGTT
CTTTCTAGTTATGTATTCTTGCAGTGTTAAGGCTTAGGGGATTGGATATAAAATATCTTGTGCATAACAATATTGGCAATAGTAATAACACCAGCTTA
AATTTATTTTTTATAGCTTTAGTAATTTAATTTATGTATATGAGTTCATGGTAGCTGTCTTCAGACACACCAGAAGAGGGCATCAGATCCTATTACAG
ATGGTTGTGAGCCACCATGTGGTTGCTGGGAATTGAACTCAGGACTACTGGAAGAACAGCCAGTGCTCTTAACTGCTGCAAAATGGTACAGTTACTCT
GGAAGACAGTTTGCCAGTCACCTGAAAAACTAAACATACTCTTTCCATATGATATTGCAACCATACTCCTTCGTATTTACCCCACCCCCAAAAGCTGA
AAACTTGTCTAAATAAAAACCCTGCACACAGATGTTTGTAGCAACTTTATTTGGAATCGGCAAAAACTGGAAATGAAATGACTTTCAGTGGCTCAATG
GACAAATGAATTGTGGTACTTTCCTGGCCGTGGACCATCATTCAGTACCAAAATGAGATGAGCTGTGGAGCTAAAAAAGACATGAAGCAACCTTAAAT
GCACAAGTGGAAGAAGCCAATCCAAGGAGCTGCATACTGTATAATTCCAACCCCATGGCATCCTGGAAAAGGCAGAACCATGGAAACAGGTTTTTAAA
AAATCAGAGATTGCCAAAGGCTAAGGGGAGAGTGGATGGCTGGGGGCAGCAGAGAGGAAAGCACCCCACAACCATCATGGCGGATACACATCCTCGTG
GCCGTTCTGGGTTTACAGCAAGAGAAACCACACCAAGAGAAAGTCCTAATGTGAACTAGAAACCAGTGATCATGCTGTGCCAAGTTAGATTTGTAAGT
CGTAAACAAGCTACTATTCTCACTGGAGATGTCTAGAGTAGAGGAGACTGTGTATGCCAGGCAGAAGGCATGTGGAAACTCTTAGTGCCTTCTCTCAG
TTTATATGTGTTTGTCTGTATGTATACATCTTTGTGTGTATGTGTGTGCATGTACACGTGCGTACACACAGAAGTCTGAAGTCGATGTTTTCCTATAT
CACTCTCCACCTTAGTTTTTCAGACAGGGTCCCTCATGAAACCTGGAATTCACCAGTTTGTTGGGGCTAACTGGCCAGTGAGCTCTGGGCATCCTCAT
GTCTCTGCCTTCTCAGCTGGGATTCCACGTGTTTGCCACCACATCCTGCATTTACACGGGTGCTGAGAACCCAAGCTCAGGTCCTCATCAGTAGGGCA
AGCACTTAACTGACTGGGCCATCTTCCCAGGCTCTTCTCTTGCTGTACAATTAAAAGTATTCTTTGAAAAAGTCTAATATGCATGCCTATATTTCCAG
CACCGAGTAAGTGGAGCTAACCTGGGCTAGACAGTAAGACCCGGTCTTGGGGGTGGGGAACACCTAACAAAAAAATAAAAACAAAACAAAACAAAACA
AAAACCAAAAACATTAAATGAAGAGCCAGGGCAGTCACAACACACGTGACTCCTCAATCTCTGTCCAACTCTGGAATTCAATAGGCTACTTTTTCTGT
TTTCCTCATCCATAAATAGAAAAAGGGATAACTGTCTCACAGGATTGTCACAGAAATTAAATGAGATGCTGCTGGATGGATTAGCAGTAGGAGCATGT
AGCAGCAGACCTGTGCAACTCTGTGTCTTTCCACTGATGGCATCATAGGCTACTGCTGGGCAAGGACCTATTCATTTCATAATCGCCTCTACCTAGCC
CAGTATGTGGTGTTTGAGCCCCCTGAGTCTGCTGGGTTGATGGTAAGAACTACCCTAGACTTCTCTCTCTCTCTGTTGGAAATTTGAGGGTTTTCTCA
ACTTTTTGCTATGAGCAAAGTACATCTCAAAACCCTTTTATTTACATAACCTAATTTGATCTGCATCCCAGGTGAAGCCAGCAGAAGAGGGCTGTTTG
CCCACGCCCACACTCTGAGACAGACAGAATCACTATGGCTCAGAGAAGTGAAGGGACCTCTTCGGGTCACAGGTATATCAGTGATGGTGATGACGATG
GCGGAGCCTCTGGCCCTGCTTCTCTAGCCCCTACCTCTGCAGACCTTTTTCTCTCTGCCTGCTGCCTTCTGCATCAGAGGTCTCTTAAAAAATTGCAG
CCTTGTCACGCTGGGCCTCGTCCTTCTGTCCGCTGTCTCGAGCGCAGCACCTTTGCCAAGTGGTCCCTGCTGGCGATTGTCAACTGCAAACTCCCAGA
TGGCCTCTGAAATCAAATATTTTATTTCCAATGCCTCTATTTTCCCAGAATGAGGAGCACACCAGTTCCCCCACACACACACTTGCTTTCGTCCCTAT
AAAGAGGTGAGGAGATGACTCTCCGTGTCCAGGAGGAAGGACTTTGGCTAAAAATAGCTGTGGCGTGTGGATTAGCCAGAGTGGTACCCAGGACTGGG
AAAGGGAGGGGGACGCTGTGGAGCTGTAGCCAGACTGGTTGCCATAGAAACGAGAGAGGAGCAGGGGAACCTGGGAAGTGGGGATGACACAGATACCA
AGTCCTAGTCTGAGCTGCCGTTACATTCAGGAGAAACAGCAGTGTCGGCGGCTCCCAATCTCAGAGGGAACCTAGGGTACTGGGGGAGATGGTGTCAG
GGACATGGACGCCAACCCCCAAGGGTCTCTGCTGCTGGCTACTCTTCTCTCCAGGCTCTGTGAGTTGAGTTGTGCGACTTGGGCTTTGGGCCCCTATT
TCTGAGCCAAGAGGGGTTTGGGTGGAGCTGCTCCCAGAGGGACTTCTCCCCGACAGACCCCTTTCCAAAAGATAAGCCCCCTGTACTGGCCAGCCCTC
TCTAGAGGGAGGTGGAGTACTCCAAGATAATGTGGTGCTCGGATCTTACTGAAAGGGGTCACAGCATGCCCAAGAACTGTGGTCGGAAGAACTGGAGT
TATTTGGAGGGAAGAGGAAGAAATGAAGACGTTGCTCTTCAGGTGGTGGACACTGCACACCTTTCCTGTCCCATGAAGAAGAGAGCTTTTCTCGAGAT
GGCAATGGCTAGGATGTCATCAGTAGGCTCCCTGGGCAGTCCTGTTCTGGGAATGATAAGACACTGGGAATCCTTCCCAATTCCTGGCCCTAGATGGA
GGTCAGATCACCTTAGACCCTACGAAGACTGTCTAGAAGCCCACCTGAAGTTAATACTAGGATGAAAGAGACCTGGGGTCTCGAGGCACTGAAAACTT
ACAGATGAGGTGCAGAGGACATCCTGGGCTGCAGAGAGGGAAAAAACAAGCCTGCTTGCTGTTGGGGGAGGGGAAGATCTTAATCTGCCATTGCCGAA
GTGTTCCCACGTCATGTCTCCTGACTTCCATGCAAAATAAGTGTGTGGGGTTACAAACAATCTTTTTGGGCTTTTTTCCTTGTGCCTTTCTTTAACAT
ACACACACCCTCCAAAGGTCTGCTGGCTACAGAACACTTGGCTCCAAAGTTTAAAAATGGAATGTCGGGTTTGTGGGTATATATTCATGCAGTTTCTC
CCTAGGATCTGCTCAAACATCCAAACCATCTGAGATCCTTATGTCACATTTCTGCCCCCACAGGGCCACCTGCTCTCCCCACTTCCCCAGCCTTCCTG
CCCCACCCCTCACCCTGAATGGGAGGAGATGGCAAATCCCAGGAAAGAGAAAGGAAGGTTGATGAGTCTTAATCCTTATTCTACAGACTTCTGTTCAT
ACGGTCCATATCTCCTAGGGGACCCTGAAAGCCTAGGAACCGACTCTGGCCATCCATCTCTCCGGGAAGATTATAACCCAGAGTGCTTCTCAGGGGGG
AAGAATTTGAAGCAAAACCAGGTGGGTTTTGCTTGGAATCTGGGCTTTGTGTGGAATGTGGGCTTTGGGACATATGGCAGGAGTGGGTGGGGTTGCTC
GTAGGGTAGTAAATGCAAATCAGGAAATTGGTAGGGGGGGTCGATGTGGGTGTTTGGTGTTTCGATTGGTCTGATTTCTTATCTCTTAGAAGAATACG
AATCTGAGAGATACTACACTACCGTAACTCTGGATCGCCTCGCGCCTCCTTCATCCTTCCCGTGGGCAGTTGAGCTCACGCGTGGCCCCCAATCTCCT
ATTGCCCACCCTTTTCAGCGTGTCTCCTGTGGGAAAGAGCCCTGGCGGGAAATGGGCTGGTATCAGAGCATCAGTGACCACGGTGAAGCAGTTAGAAT
TGCCAGTCGGAAGTTCCCAATCCTGACGACATCCAACCTTTGCACACTGGAGGTTTTTGTGCACAGTCTGCATTGCTTTCTCCTTGGGAAGTCTGGGG
TGGAGCGGAAATGTAGCAGGAGAAAGAGTGAGGCCAGGCAGAACACCGAGGGAACAGTCTTCAGGTGGGGCTTCTCGCACGATGCTGAAGACTGCTGG
GGGAAGGGATAATTGCCAGGGAAAGGGCTGTGGAAGTCCTCATCGCAGGGAGGGCTTTGCATGGAGAAGGAACTGCCAAGAAGTCTACCTCTTCAGTA
CCCTAAATGTCTGATCCGGGGTGCCTGTGAGTTGCTACATACACCAGCTTGAGGTAGTGACGCTGAGATCTCTGACATCGAGATGGCTAATGCCTCTT
TTCTTACTGAACTTCGACACCCAGTCTGTGCTCTTTATCCTGTGTAATCTGTACAACTCTCTCTCTCTCTCTCTCTCTCTCTCTCTCTCTCTCATAAT
TCTTTATTCTTTTTTAAAAAGATTTATTTACTTAATGTATATGATTACACTGCCGCTGCCTTCAGACACATCAGAAGAGGGCATAAGATCCCATTACA
GATTGTTGTAAGCCACCATGTGGTTCCTGGGAATTGAACTCAGAACCTCTCTGGAAGTGCAGGCAGCGCTCTTAACCCCGCTGAGTCACCTCTCCAGC
CATACAACTTTTTCTTAACCATTGTTTTATTTTATGTAATAGTTTGCCCTCATGTACGTCTGTGCATTACCCTCGGAGGCCAGCAGAGTGCGAGTTAC
AGCCGGTTGTGAGCCGACTTGTGGGTGCTGGGAATCGAAATCAGATCCGCTGGAAGAGCAACCAGTGAATCATTTGAGCCATCTCCCCAGCACTTGTG
CCCCAACTTTCTGAGATTTATGGGATGTTAGGGATTATCGTTCCCAATCCACCAGTGGGGAAAAACTAAGGCTAAAGAGACAGGAAGGGAGATTGTCT
CACAGCATTGGCCCTGAGTTCGGGGCAGATCCATCAACTCGGCACACCTTTATTAAGACCCCGCAGGATCCCCGCTGCGGCCGCCATGGCGCAGGAGA
ACGCCGCTTTCTCTCCCGGGTCGGAGGAGCCGCCACGCCGCCGCGGTCGCCAGCGCTACGTGGAGAAGGACGGTCGCTGTAACGTGCAGCAGGGCAAC
GTCCGCGAGACCTACCGCTACCTGACCGACCTGTTCACCACGCTGGTGGACCTGCAGTGGCGCCTCAGCCTGCTCTTCTTCGTGCTCGCCTACGCGCT
CACTTGGCTCTTCTTCGGCGCCATCTGGTGGCTCATCGCCTACGGCCGCGGCGACCTGGAGCACCTGGAGGACACCGCGTGGACCCCGTGCGTCAACA
ACCTCAACGGCTTCGTGGCCGCCTTCCTCTTCTCCATCGAGACGGAGACCACCATCGGCTATGGGCACCGCGTCATCACCGACCAGTGTCCCGAGGGC
ATCGTGCTGCTGCTGCTGCAGGCTATCCTGGGCTCCATGGTGAACGCTTTCATGGTGGGCTGCATGTTCGTCAAGATCTCGCAGCCCAACAAGCGCGC
CGCCACTCTCGTCTTCTCCTCGCACGCCGTGGTGTCTCTGCGCGACGGGCGCCTCTGTCTCATGTTTCGCGTGGGCGACCTGCGATCCTCACACATCG
TCGAGGCCTCCATCCGAGCCAAGCTCATCCGCTCCCGTCAGACGCTCGAGGGCGAGTTCATCCCTTTGCACCAGACCGACCTCAGCGTGGGCTTTGAC
ACGGGGGACGACCGCCTCTTTCTCGTCTCACCTCTCGTCATCAGCCACGAAATCGATGCCGCCAGCCCCTTCTGGGAGGCATCGCGCCGCGCCCTCGA
GAGGGACGACTTCGAGATCGTAGTCATTCTCGAGGGCATGGTGGAGGCCACGGGTGCGGGCAGGCTGGAGGATGGGAGCAGGGATGCAGGACAAGGGC
AAGAAAAGCAGCCAGGGGAGGCGCAGAAAGATGGACAGAGAATGGAGTGTAGGGTGACAGGCCTGAGGGGTAGCGGGGGCCGGGGAGAGGACGGGAGA
TGACAGGGATGGACAGGGTGACTTTGCAGAGTCAAGAAAAGCTTGGAAGAGGTCTATGAAATGGCACTAGCTTGAGGCCCTGACCTGACAGCTATGTC
ACTTTGAACTACATTTTACATCTCTGAATTCATTTAAGCCCAGCAAAGCTCCCCTGGAGGTTACTTTTGACTGTGCTCGGTTTTCAGAGAATGAGTAG
CCCCAAAGAAAGGTCCCATAAATAGCCCGCTGTCACAAGCCAATAAATAGCACAGCCTGGGTTGAACATAGGACATCTATCTTCAGTGTTTCCTGGTA
CAGTGTTGGGATGAAGGTTAAGTGCAGGGTTCTTGAAGCCCAGAGGTCCATAGCTCTGGAATTTAACTGACCTAAGTAAAAGGGAGGTAGGTAGGAAA
AAGACTAGTACTGGAGCAAAAACAGGTCCTTGAAGAGGTCCTAGCCGTCAGGGAGCATAAGGAAGACGCAGGTGAACCAAGAGGCCACTAGGAGGAGC
TGCGGAGCTGCTACGGACAGGCTAGCTCCCTGCTGCTAGCCTTGAAACCTGGCTCCTGGGCCTAGACAAAAACATCATCTTCTCCATGGCCACCTCAG
GTCTTCCCACTCCCCTCTCCTCCTTCACTCCAACTAGGCTGGTTCTAGCCCATGCCCATTCCACACTGCTCCCTCTGTCTCTGCGCTGTCCCTCTCTC
TGACACAATCTCGGACAGGTTTCTATCAGGGACTTTTTCATCTGCCTTCTCTTCCCCCTCTGCCACTGCCTCCACTTTGCACCTAACCCTACTCCCCC
AAGCCCTACCTCTGCTTCTCAGGCCTTCTCCCTGCAGAGGCCCCGGTGGCCTCTCTTTCCCTACGATCCCTGATACATCTTATTCCAGCTTTGCCAAA
GAATACCAATGACCCCAAGATGTCTCAGGGCCAGACTTCCGATGTCAGAGCCGGTCTCTGATTAGTGAATGCTTACTCCTCTGTTTTTGAGATGGATT
CCGGTTTGGGAAGATTCTGAGGTAGGAACAAAATGATCTGCCCCGAGGGGAGGGTGCACAAACCCAACAGAGAAGACAGGACACAGGCTCAGGGCAAG
AACTGGGAAGGGGCAGTGTAAAGGACATGGGGATGGGAGCTTGCTTGACTTTTCTAGAGATAAGGCTGGGAAGGATGGTAGTATTTTGGGATTCAAAC
TGCTTTTGAAAAGCAAGAATAATGAGCCAAAACCCAACATGATGACATTTAAGGGGAATAAATATAAAATTCTACATTTAGGCTTTAAAAAAATCACT
TATGTAAGCACAGCATGGAAAGGCTCCGGTGGAGAAAGAACTGGGGGTTTTAGTTGGCCACTGGCTTTGCTGCAGCAACGTGATGCAGCTTCCAAAGG
CGTTTATGTAATGTAATCATGGGCCCGCTTCACCAAAGCATCTGGGCGAGAAGCAAGAGATAGTAAGCCTTCTTTTATGCACAGATAAGGCCACAGTT
GAAAAAGCACTTCAGATGAGCCCTTACCTGGGCCTGGTGGCCATTCTGATTTGCAATGAAGATTGTAAGCTTTGGGGGAGTCAGATGAAGTAAGAAAT
GGCCATGAGTGTTCAATCTGAGGAAGAGAAGATGTAAGGGAACCCCATATTTACACTCAAGGGGGTGTCAGGTGGTAAGGGAATGGAACCAGGGGCCA
CGGGTCCTAGGAGACAGATTTTAGTTTATGTAAGAGAAAACCCAGAGCCAAAGAGATGTCTCAGCTTGCAACCACGCCTGACTACTGACCTGAGTTGA
ATTACCAGGTCTCACATTGGGGAGTCAACTGTCTCCCCAAGTTGTCCTCTGACCTCCACATACATACATATGCACGCATATAGACACATAAATGTAAA
ACACATTTGTAAAGACGATTGGCACGTTGCACAAAGGACTGGACTTTTAATGAGATGGTGAGCTTTCAATCCTGGGGTGTAATCAGTTCAGCCCATTG
TCTGGGAATGCTTGGGGGTGGGTGGAGGCGGCTCTGTGGGAAACAGGAAGGTTAGGCTTAAGGTTAAGCTTCTCAATGGAGAGTAGGGGAAAACATAG
GCTGGCAGATAGAGAAGAGGGCTAACTAAAAAGAGAGGTGGGACTCTCAGAGAGAGAAGAGGGTTGTGGGATGACAGACAGGAGAAGGAATCCTCTGT
CAGGGGCCCCTTTGACTGATGCCGCTTCTCCTCCCCCCACCCCCCAGGAATGACGTGCCAAGCTCGAAGCTCGTACCTGGTGGATGAAGTGTTGTGGG
GCCACCGGTTCACATCCGTGCTCACCCTGGAGGATGGTTTCTATGAGGTGGACTACGCCAGCTTCCACGAAACCTTTGAGGTGCCCACACCCTCGTGC
AGTGCTCGGGAACTGGCAGAAGCCGCGGCCCGCCTTGATGCCCATCTCTACTGGTCCATCCCCAGCAGGCTGGATGAGAAGGTGGAGGAAGAAGGGGC
TGGGGAGGGGGCAGGTGCGGGAGATGGAGCTGACAAGGAGCACAATGGCTGCCTGCCACCCCCAGAGAGTGAGTCCAAGGTGTGACTGGTTTCCTCCC
ACCCCCTGTGGCAGACCAGGGGGCCGGACTCAGGTACACAGAAGCTGCGAGTGGAGGTGGAAGAAGAGGAGGCAGGCAGTGTCCCGAGGAACAGCTAA
AGTTGGGAGAGGCCCGCTGAGTCCAGGATCGAGTAGGGAAGGCTGAGGTCCTGGTTTGAAGAGAGAGGGTTGCAGGGCGGGGTGAGAGAACATGTCAG
TCTGTCTGTGTTTGACCTTCACATCGGTTCATGGGTGGATGGATGGACAGAAGGATGGGCTCATGGGGGTTGATCGGGAAGGTGGAGCAGATAGAGAC
AGCCAATGGATAATCGCTCAGGTGGTAAGTGGCTTGGCAGTCGATGATCGTCACCTGCAGCACACCTTTGTGAGAAATCCATGGGCATCCTTTTCTTC
CAGATATAGGTAGCCTCAAACCAGGGAGCGTGGCTTAGGGAGCAGGCTGTCAGGTGGACTACCACCCCCACTCACCTCCCCTCAACTGGCCTCCCTAT
GTGTGACACGCCTGCCTAACTAGAGAAGAGAGCACTGGGTAGAGGTGGGCACAGGTGTGGGTGCCCTCCCCAGCATCACTGTCCCATGGCGAGAGGTC
AGAAAGGCAAACAAGCAATGGGGGTAGATGCTGAGCAGGGAGGGGCCCTGAAGCAGGACCTGGGGACAGCCAAGGACAACTATTTTGTGAGAGAGGAA
TGAAACCTTGCAGGTCCTGCCACAGAAGCAAGAAGCAGAGGAAAGGCCATGGAGAGACTTAATAAAGGGTTTTACAAGGGTACCTGGATCCCAGGGGG
AAGTAGTTTATCCTTGGGGCACAGTGGCAGGGCTCATTCAGAACGGTGAGTAAGTGTCAGGTGTGATATTCAAAGACCTGGTTCTTAACACGAGAGCA
CAGCGAAGGTGGAGGTCAGAAATAACTCCCAGCCACTGAAGGAAGTATGGCTTCAGTCTGGAGAGCTCAGAAAAGACTCGACCCTAGGAGCCCACACA
AGCGGTTATAGCCACAAGTGAGAGGGCATTAGGGACAGGAAGCTAAGGATTGAGTAAGGCAGTGGGGAATGGTGGGAGCCAGCAGTTACAAAGCTTTA
CTCACCTGGATGGGCTTGTTAAAACACAGATTACCAGCCCCACTCCCTGCATTCTGACTCAGTAGGTCCGGGACGGAAACCAAAAAAAAAAAAAAAAA
ATCTGCACGTCTAACTAGTTCCCAGACCTAACAGGTTCCCAGATCGCGGTGACACTGTCTGTCTGGGGACTGCACTTGGGTGAAGCATCTAAGCGGAA
GAGAAGCTGGAGGAACTGAAAAGCACCCCAGGTTCCTCAAGGAACAGAGAAACAAGAAGGGAAATGTTGGGGAGAGGGGACCCAGGTCCAGACTCGAA
GGGCTTAACTCTGGGTCCAAGAAACGTCATTGGTAACTGGCCAGTGGCACCCGAGAGGGCAACAGAGATAGGAGAAGGCCATTTAGGGACCCCCAAGG
AGGCAGTGGGGGGTCTGTAGCTGAATTGGCCTTACCACAAAAGACCAACTCTCTTAAGAGACTCACAAGGCAAGACTGACTAGGGGAGAAAATGGAGC
CTGTACCTACAGGTGTCTGCTGTCTGCCACCTGTCCTCCCAGGACAGGGCACCCTGGAGACACATTCCACCTCCACTGCATCCTTGTCTTGCCCCAGT
CATCTTGGGATGGTTGAGGGGACAGCAACAGCATGGCAATGGACCTGAGGCTGGCCCCCCTGGAGCTAAGTGTAGCCCAAGTGAGCACGTAACCTGAT
AGGACTGGCTCAGACTCTGGCCCTGGCTATACCCATCCCTGCCCTCGAATAAAAGTCTGCTGCTCTGTCCCAGGCTAAGAAGCCAGCATCCAATGGGG
CATCGAGGCCTCCCTCCCAGTGCCCAGCTCAGAGTGGGTCCACGCAGAGAGGACTCAAGCTGCCTGTTGCCTCTCCCCTTCCATCTAGCAATGGCCAC
AGGTTTCGGGACCAGCTGGGTCACTCTCAAAGATGAGGTCCACGCACATGAACCTGCTGGGATCCCACGAACACATATTGGACCTGAGCACAGGGACT
GAGCAGGGTTTGAATTCAGAGAAAATCGAGGAGTCTAGACAAGAGGGGTGGGGTGCTTGGTATCCGCACACGAAGCAATGGAATGGAGACATTGAAGC
TGTTCCTGGAGGTCACTCAGGGCACCGCTGTCCAGGGCACAGCCAGGAGACCTGTGTTCTAGCACCAATGCTGATTGTCACTAATTACCTCTATGACT
CTCAGCAAGACCTATTACTTCTCTGGGCCTCAGCTTCCTTATCTGTTAAAAAAAAATGATATGTTGGCAAACTCAATAATGCTCAATAAACTTTCAAC
TACTGAATGAAAAAAGGTAGACTGGATGCCGCTCAAAGTATTAGGACAGCTGAGGCTCTTAGGACCGGAGAACCCTTTAGGCGGGGAGTTGCGGCTAG
CCAGCAGGCAAGTCCTGGCATCAGATGTAAGCAGATGAGGCGGCTCTTGTGTACACAGAGGACACAGGCTCTCCCAACTGCTGCTGTCCTTAAGTAGG
CAGCCGTGTTCTGAAGCTCCTATTCGGCTGCTGTCAGAGAATAATTAAGGGCAGGAGGAAAAAGACTGAGGCCCCAGGGCCTGTGGGAGGAGTCTGGT
CCAAGACTAGTTCAACCAGGAGAAATGGACCAGAGGAGGGTGTGCCCCAGTCTGGAGAGCTCAGAAAAGACTCGTCCCTTGGAGCTCTGTGAAAGGGG
CAAAGCTCAGCTGGAACTCACCCCTCCTCTTCCTAGGTCCCCCTTCCCAAATAGAAGCCCCATTAGGACTTGGCTCAGCACAGACATTTTGGACAACA
GATGGGACCCCGGCATCCCCTCATGCAGTTGGTGGGTAACAAGGCCCACGAAGGGACAGATGGTGTTTATGGTGGGAAGAGAGGCCCGGGTTGTCCAG
CAACCACCCTACTACCACCCCACCCCCACCCCCGATGCTGCCTTTTATAGCTTCACCGCAAGAGAAGACACAACAGGCCTCGATTTTACAAAACCAGT
TTATTCACATTTTAGAAAAACTAGTTTGAGGACAGGAACTGGCCTTCCTACAACATGAGTGTGGGACTAAGAACGGCAGCCAGGAAACTTGAGGGAAG
GTGGGGACAGGGGAGCCATGTCTCCCACTCTAGGTGATGGCTGGTCAAATAAATTAAAGGTGGGCTGGACAGAGGGAGAGGGTATCCAGGCAACCAGA
GGAGGGGTGGCACTGGCTGGAAGACAGTCAACACCTGCAAGAACTGGAAAGAGCATGTGGAGTCGGCTGAGGAAGAGGCTCCCTTTGACCCTTACCCT
GCTATACGATCCTGCAGGACTGTGAAGCTGGCTGCTTCTCCCCCTGATGGTGCCCAGGTACAGCTCAGCACAGGAAGCCTGAGGAAAGGCAGTTCCTT
TCCCTCACCTTGGGGTGCTACAGATCACCGCTTCCGCATCCTCTTCATAAAGCAGCAGGTGATGGTAGCCAGGACGCTGGCGCCGGTAACTAGGGCGA
CTCCTGTACCCACCAACAGGGGCACAAATAGGGTATCCACAGCTGGCAGAAAAGAAGACAGGCTCTGCTCAGAGAGTACCACGGTATCTGACACTCTC
CCCTGCAGATTTTCTAGACTCAGCCCTCCCCAAGGGAGAGCTGAGCGCCAGTCCTGCCTACCTACACTTCACACACAAACACAACCATCCCCCATCCC
CCATCCCCACCCCCTCCCCTCGGTCTCAGCACTCAGGCCGGCTTGGGGCCCTTCATGCAAAGGGATGTGGAAAAAGGATTGCAAGGGAAGACAGGAAG
ATGGAAAGGGGCAAACAGAGCAGGAACAGGTGGGTAGATGGTGGCTGTCACTCACCATGCGTGTAGGGGTAGACTGTAACAGGCCCGGAGCGCGCACT
GCCTGCCTGGTACCAGCTGTAGTCCGCATGCTGCACCCAGGCACTTGGGGCACAGTGGTATATGCCTTCATCCTCAGGCCCCAAGCCATGCAGTCTTA
GCCGATGACTTCTGGGTCCCACCAGCTCCACACTGACAGGACCCCCTCCAGGCCGGACTCCCAGCTCTGCCACACCGTCCTGACCCACTCCACCCACA
AGCTGAGCAGGGCCAGAGCTCAGCTCGCCCTCCTCTGGCCTCTCCACCCACCAGCTGGCTGCTAGTCGCAGCCCTGGGGGGCCGCCCCGCACAGAGAT
GTTGCATAGCAGGGAGGCCGTCTCTCCCCGGTACACAGTGCCCCCTGCTAGCCACGCCACGGCCTCTAGCACCACGCCTGCAGAGCAAAGAACACGGG
GGTTACCAGGTGAAGGCCCAGGGGCTAAGAGGTTAGGAAATAAATTCTATAAGTTCTGAACCCCGTCAAGGGCTCAACATCCTCTTACCTTCTTCTCT
CACATGCACAGGGAGAGGCCGGGAACGAGCACTGGCCGCTTCACGAAGTCGGGTCCCAGACCCTCGAACATAGGCTTTGGCGAGGCAGCGGTAGGTAC
CTGCATCAGCGGGCCTGGCAGCCTCCAGCCGCAGTCGGTAGGTTCTGGATGCTACTTTCTCCATGGCAATGTGCCGGTCCTCATAGCCAGGGCCCAGG
CTGCCTATACCTTCCGTGTCTAGCTGGGCCACCAGGCGGCCGGGTCCAGGAGCCCCTGCAGGGGCCATCTCCCAGCCCACAGAGTACGCAGCATGACG
GCCTGGTGGGGGCAGTGCACCGGACACATTGCACAGCAGTTCTAAGGGTTCGCCTGGGCCAATCCGACGTTCACCAGGTCCCACGGTCACCGCCAGCT
GGCTGGCTGAAACACAGCAGGAGATGGGAGGAGTCACTGAGATGCCTGGGCCCCCCACCTGTAATTCTTCTTTGCAGAAATTTAGAGGCCTCTTATAT
CTCCCTCACCCCAGGACCCGAATTTCACCCTTCCCCCCATAGCCTTTGTATCTCCATGCTTGTGCGGCACTCCCGATGCCCAACTGAGAGACACCCCC
CCCCCCCCAGTGGGCTATGCTGCACTCACATAGAGTCTGCACATCAACATGAGCCAGGACTGCCCTCTTCTCTGCGACCTGGACCCAGGAGCCGTCAG
GATCCTGAATCCACTCAGCGGCCGTACAGTGGTAGGTGCCCGAGTCTCCAGCCTGGGCACCCCCAACCACCATTCGGTACCGATCAGTCCCTTCCTTG
CTCAGCCGAAGCTCCCCAGAAGCTAGCCTCTCAGCGTAGGGCGCTCCAGCCTCCACCGCCATGTCGGAGCGCAGTCCCACTACTTCCTGTAGAGTGGC
TCGCCCCACTGGCGCCTCCGGAATGGCTCTCCCAAAGGACACCGACAGGTGTGTGTGTTTCTTTGTTTTGGTCTGAGCCAGGCAGCCCAGCGCAAGCT
CCTGCCCCTCGTGCACTGTGAGGCGTGAGGGGGAGGTGGCAGCCTGGCGCCCTCGGGGCCCTGGAGGGGCAGCAGATACCTGCAGCTCATCTGGAAGA
ACTGGAGAGAAAGGCTTTAGTGAGAGAGGGCTTGGAGCAGCATCCCTCCTGTTTCCTGTGCGTATCCTGTTTCACTACACACTCTCTAGGCTTCTAGA
ATGTAAGAACTGTGCTCTGTAGCTTTTCTTCTATACCGCAGAGATGCCAAGCTTGGTCTGGGCACATCAAGATATTCAATAACTACTTGCTGAACGTC
ACAGAGCAAGCCTACTGACCCCTACTCTGATGTCTAAGACTGATCCATTTTAAATACTCAAAAAAAGTAATCCTGTCTTCCTTCTCTAAAGATAAAGA
GGCTGGACCTGATGGTGCCGGCCTATAATCGCAACTACCCAGGAAGCTAAGGCAGAAGAGTCAAGTTCAAGGGCCGTCTGGGCTACAGAACAAGTTCA
GTGCTGGTCTGGTCAACTTGGAAAGTTTCTGATTCAGCCCATCCCCCCCCCCAAAAAAAAAAAAAAAAAACTGCTGGAAAGACTGGCTTGATGGCACT
GGAGCTGACACAATACTGCCTGAGCTACACAGTGAATTGTGGGAGTTTTGTCACCAGTTTCAGGCCAACCTAGGCTAGTTGTAAGCTAGCCTGGGCTA
CAAGAGTGAGCCCTCAAAAAACAACACAGGGAATATAGCTTAGTAGAGTGCTCCCTTGGTATGTTCCATGCTGTAGGTTCAATTTCCAGTACTGAGAA
GATGGGGGTGGGGGGAGAAGAGGAGAAGGAAGAAGATGACAGAGAAGGAGGAAGACGAAGCAAAAATAGATCTGAGCGTGCTGGCTTATACCTATAAC
CCCAGTGCTTGTGAGGCTCTCTCACCACCTAGCTCAGAGCCCAGTACCTCTCAGCTCCACCTTGGCACTGTAGTTGCCCAGGTACTGCGTATCCGTGG
AGGGGGTGTAGCACTCATAAAAGCCAGAGTCCTGGGCCTGCAGGCGAGCAATCTTGAGCACCACCGAATCTCCCTTCAGGCGCTGCACCTGCAGGTCA
CCAGATGCCACACGAGGCCCAAAGACAGCATAGGAGAACTGGCTATCCTTGGTGCTGACAATGCCCAGGGACGTAGCTGGGGCCTCTGGTCTGTACAT
GAACCACTCGAAGTCTTGCTGGGCAGGGCCCTCATAGTCACTCACGTTGCAGGAGATAGAGACAGCGGTGCCAGCCACCCGGTAAAGAGGTCCCCTGG
GGACATGCACCTGCCGGGCGTAGCACCTGGTTCCTGTGGGGTAAAAGCAGAAAGAACTGGAATCTTTTTAGAGAGAACGAGTCCCCACTTGATGGCCA
GTATCATCAGCACCATTCTTGACTGCTGCCTGTGAAGGAAAGGGAAACCTAGGGATGGTTAGAACATAGCTGGGCAAAGACACAGATGGGAACACAAG
ATGGGACACGAGACACCAGCATCCCAGCACATCACGTAGGTTCAGATCCACCTGAACAGAGGAATACTCTAGCTAACTAGAGGCAGAGCACCTACATG
AACAGAGGAGTCCATCCGGAGTCTGGGGTATAGACAGCCCGGTAAGTAAAATGCTTTCCATGCAAGCATCAGGACCTGAGTTCAATTCCCGGCAACTA
TGGAAAAAGCTTGGCACGGTGGCTGGTGAGTAGCCACATAAGCCTGACAACCTGGGTTTGAGCCCCACAAGGGAAGGAAGGAACTGAATCCTGAAAGT
TGTCTCCTCTCTCTCTCTCTCTCTCTCTCTCTCTCTCTCTCTCTCTCTCTCTCACACACACACACACACACGCACACGCACACGAATAAATGCAGTGG
ACGGTTCCTGAGCCATGACCCCTGAGGTTGACCTCTGACCTTTACACAGACACCTGCACCCATGCATACATGTGGACCCTCATACGCATGCAATTGGA
AATAACAAATAAAAGAAGCATGCCTGATTCCCAACTCTCCAGCTCGTGGCTGGATCTTTATCGCTCCTACCCTGCCATGTGTGGCGTCTCTCCGGGCT
CAGAAAGAACTTCTAGCTAAGGGATTCTGAGCCTTTTGCTGGAGGGGAGCTGACAACATCTTACAGAAAGGCTGCTTGGCTTAGCTCTGTCACCTGGG
CTAGACAATGGAGCCAGTGGCCCAGGCTGGCTGGGGTACTGCACTGGATGGGGCACCATGCTGGACAGGGCACGGACCTAGTCAGTCCTCACTACACA
ATACCCTCCCCACTACAGCTGTGCCATGAGCTCACTGCTTCTCCCAGCCCACAAGGCTACACAGGCAGCTGTGGCTTCTGGGGCAAGAACCAGGCTCT
GCCCAGGCCTGGGGCAGAAGATCCCTTCCCCGCCCCCAGAATCTGTCAACCTTCTCGCTAACCCAGATGATGCACTATGCACAGCCCCCCAAACAAAA
GCTTTCATTTACATATGATTTGCCTATCTGCGCAGCATTTGCATAGACCTCCTTTTAATAAGGAGACCCCAAACACCTGCTGCCCCTTCCCCCTTCTC
TCTCTCTCCCTCCCTCCACCCATTTTACCCCACCACTGTTCCAACAGACAGCCGGTTCCAAAACCTTGGACTGTTCAGCTTCTTTCTCCTCTCCACTA
CTTCTAGGTAAGACCGCAGAACCCTAGAATGCAAGACGGGCTGTCAATCATTCCTCAGATGAGAGAAATGAAGAAACTCCCAGAGAGCACCTCTAGGG
AGCATTGCCACTAGGTTCTAACCACAGATGTGAGTCTAGAGCTCTCTCCCAGCCAACACGGAAGGCCTGGCCGTGAGAACCATCTCTCTTCTAGATGA
GAAAAGTAAAGTGAAATGTGACATTGTGGCCAAGTCCCTGCCCTCTCTGGAGGGCCTCTACACACCCCTGAAGAGGGACAAAACCAAGAAGGCGGAGA
TGCTTCCAAGGAGAGCCCTGGTTTACAAAACTGTAATTTCCCGGATTTCCAGGGGTAAGTCCACAGCTTGCTACTGGCCCTGGAGGAAACCACCCAAG
AGCCTGAGAGCCTGCTCTACAGTTCTCGCTCCTTTCCTTCTTCTGGCTACGTCACGCAGAGAACACATGACCACCGCCTGAAGCAGAGGCTAGAGTCA
GAAAGCCAACATGAGACCAACCCTTTCCTCTCCTGGATCTCAGTTTCTATCTCTCAGAACTAAGAGCTCCCAGTTCTGATGTTGAACCCGTGAGTATA
TGTGTGTGACTCAGGCACATATCGCTCCAGGCACATTTCATAATCAGGAGGATGGTTAAGGCAGCCAAGCTGACACCATCCTGGCTGCCCATGGGTAA
ACTCTGCTGAGAAGGTCCCCTCCCACCTTCCTGGCTCCACACAAGCTGATGCTTGGAGATCCCCATGGGAAAGTTGTCCCCACAGCCTCAGGACATAG
GCTGCTACAAGGTTCTCAATGGCTGGGCTGCCTCCTTTCTAACAGCCAAAGGTCTGCTTTGAAGTCAGTTCTGAGTTCAAATCCACCCCCACCCTCAC
TGGATACACAGTGGTATTTCACGACATCTGTGAAGTGAATGCCAGTGCTGGCTGCAGTGGGCTAAAATGACATTCAGCTCCTCTCCCGCCCCTGAATA
ACACTCACTCCTCCCAACCACCCCTGGGCTCATCCCTGCTCGGGTTAAGCCCAAAGGAAAAGAAGCAATCGCTAGGCAACCAAGCCCCACAGCTCCTT
CAACTCCCTACATCACTCTGCCTCCCGCCTTGTTCCCAAAGGAGTTTTCATCCTGGCCCCAGAAGCCCAAGGAACCATTCTGTACAACACTGCACAGT
TCTGGTATAAACCAGAGAAGGAGGTTGGGGTGCCCCACTATAGTATCTTCTCCATATGCATATCACACACACACACACACACACACACACACACACAC
ACACACACGTTCCTTTCAAGGGCTTCAGTCTCCTGGCAACTGCTCCATGCCATATCTTTCCCAGACCACCTCCTACAGGGAGCCCTCCAAGTCAGACC
CCAAACATGGTAATGTTAGCAACCTCCACAGGCCTCAACACACACACACTCACACTCACACACACACACCAGACATGACGCAAGGTTGGCCCAGAAAA
CACACCATCATAAACACCCACCAGGACAGACACTGGGTGCTTAGAGATCCCAGGTTCAGTTTCCATGGAGCCTAGTTTCTCCTGAGGCAGGGATGTTG
GGACCAACTGAGTCTGACAACCAGGCAAATATCTGGGAGCGTGGAAGGGCAAAGAGGGAACTGGCCCAGGGTGGAGACACGTGAGGGAAGAAGCCTCA
GATGGTGACATGTTATATTGGGAGGTGGGGGTGTTGGGGAGACTTTTTTCAGAGATCGTGGTCAGAATCAGCCCCTGGGCCTCCAGCCAACTCTGGGC
AATTATGAAGACCGCCAGGCACTGCCCACGCAGAGCAAACACCCAAAACCAGGCCTTGAGCCGAGAGTGGGGCAGAAGGTTGTCACGGTATTTGGTAG
CAACGACCCCAGACGCTGGGTGTAACCGATGAGAAGTGGTGCCTGCCTCCGGAGGCCCGATGGTGTCTCAGGGGATACCTCAGTAGGTCGCCCATATG
CCCCAGCTAGGAACCTAGAGCGAGGACACCACCACCCTCCCCATAACTGATTGGGCAGACAGGCGCAAAAGGAAGCGAGACGCCGAGCCCAGAGACAG
TGGAGGCACGTCTGTTGGAGAAGTAGGGATGCAACCAGCTCTGAAATGCTAGGAAGGTGGGCTGGTGGGCTGCACTATGTTAGGCACCTACCCGGCCG
GGACAGGGACGCGGCGACCACCACCTGGCTTACCAAGTATTAGCAGCAGCAGCAGGAGCGAACTCAGCGGCGTGGGGCTAGGGACGCCCATTCTGCGT
AGGCGGCTCTGGGGAGACTCCTGGGGGCGGCGTAGGCTCTGGGGGGCCAGGGCCGCGGGGGGCGCATGCCCAGGTGGGGGGCAGAAAGCGGAGCAGTG
AAGCGTGGGTGCGCAGAGCCCAGCCGAGCGGGAGCCGCCAACTCCCCGCCCTCCACCCTTCTTCCCCTCCTCCCTCCGCTCTTCCCGCCCTCCGCAGC
TCGGGAGACCAGTCCCAGCCGCGCCCCGCTGCCCGGCCCCGCCCCCGCCTCGCCCCGCCCCAGGCCGTCGCCTCGGCCAGACTTCGACCCTGATGGTG
GCTCCGCCTCTGGCCTCAGGCTGGGCGAACTGGCGGCACCTGGGCTCCTCTATCCCCATTTCCTCGCTCAGAGGGCACCCCGCCCTGCACCTGCCAGC
CTTCCAGGGAGAATGGGGTGCTTTCAGGGCCTCTGGGGATGCATGATGGGGTGACTGTGGTTACGCACTCAGAATCCAATTGGG
MOUSE SEQUENCE—mRNA
CTGAGCTGCCGTTACATTCAGGAGAAACAGCAGTGTCGGCGGCTCCCAATCTCAGAGGGAACCTAGGGTACTGGGGGAGATGGTGTCAGGGACATGGA
CGCCAACCCCCAAGGGTTTCTGCTGCTGGCTACTCTTCTCTCCAGGCTCTACTTCTGTTCATACGGTCCATATCTCCTAGGGGACCCTGAAAGCCTAG
GAACCGACTCTGGCCATCCATCTCTCCGGGAAGATTATAACCCAGAGTGCTTCTCAGGGGGGAAGAATTTGAAGCAAAACCAGACCCCGCAGGATCCC
CGCTGCGGCCGCCATGGCGCAGGAGAACGCCGCTTTCTCTCCCGGGTCGGAGGAGCCGCCACGCCGCCGCGGTCGCCAGCGCTACGTGGAGAAGGACG
GTCGCTGTAACGTGCAGCAGGGCAACGTCCGCGAGACCTACCGCTACCTGACCGACCTGTTCACCACGCTGGTGGACCTGCAGTGGCGCCTCAGACTG
CTCTTCTTCGTGCTCGCCTACGCGCTCACTTGGCTCTTCTTCGGTGTCATCTGGTGGCTCATCGCCTACGGTCGCGGCGACCTGGAGCACCTGGAGGA
CACCGCGTGGACCCCGTGCGTCAACAACCTCAACGGCTTCGTGGCCGCCTTCCTCTTCTCCATCGAGACGGAGACCACCATCGGCTATGGGCACCGCG
TCATCACCGACCAGTGTCCCGAGGGCATCGTGCTGCTGCTGCTGCAGGCTATCCTGGGCTCCATGGTGAACGCTTTCATGGTGGGCTGCATGTTCGTC
AAGATCTCGCAGCCCAACAAGCGCGCCGCCACTCTCGTCTTCTCCTCGCACGCCGTGGTGTCTCTGCGCGACGGGCGCCTCTGTCTCATGTTTCGCGT
GGGCGACCTGCGATCCTCACACATCGTCGAGGCCTCCATCCGAGCCAAGCTCATCCGCTCCCGTCAGACGCTCGAGGGCGAGTTCATCCCTTTGCACC
AGACCGACCTCAGCGTGGGCTTTGACACGGGGGACGACCGCCTCTTTCTCGTCTCACCTCTCGTCATCAGCCACGAAATCGATGCCGCCAGCCCCTTC
TGGGAGGCATCGCGCCGCGCCCTCGAGAGGGACGACTTCGAGATCGTAGTCATTCTCGAGGGCATGGTGGAGGCCACGGGAATGACGTGCCAAGCTCG
AAGCTCGTACCTGGTGGATGAAGTGTTGTGGGGACACCGGTTCACATCCGTGCTCACCCTGGAGGATGGTTTCTATGAGGTGGACTACGCCAGCTTCC
ACGAAACCTTTGAGGTGCCCACACCCTCGTGCAGTGCTCGGGAACTGGCAGAAGCCGCGGCCCGCCTTGATGCCCATCTCTACTGGTCCATCCCCAGC
AGGCTGGATGAGAAGGTGGAGGAAGAAGGGGCTGGGGAGGGGGGCAGGTGCGGGAGATGGAGCTGACAAGGAGCACAATGGCTGCCACCCCCAGAGAG
TGAGTCCAAGGTGTGACTGGTTTCCTCCCACCCCCTGTGGCAGACCAGGGGGCCGGACTCAGGTACACAGAAGCTGCGAGTGGAGGTGGAAGAAGAGG
AGGCAGGCAGTGTCCCGAGGAACAGCTAAAGTTGGGAGAGGCCCGCTGAGTCCAGGATCGAGTAGGGAAGGCTGAGGTCCTGGTTTGAAGAGAGAGGG
TTGCAGGGCGGGGTGAGAGAACATGTCAGTCTGTCTGTGTTTGACCTTCACATCGGTTCATGGGTGGATGGATGGACAGAAGGATGGGCTCATGGGGG
TTGATCGGGAAGGTGGAGCAGATAGAGACAGCCAATGGATAATCGCTCAGGTGGTAAGTGGCTTGGCAGTCGATGATCGTCACCTGCAGCACACCTTT
GTGAGAAATCCATGGGCATCCTTTTCTTCCAGATATAGGTAGCCTCAAACCAGGGAGCGTGGCTTAGGGAGCAGGCTGTCAGGTGGACTACCACCCCC
ACTCACCTCCCCTCAACTGGCCTCCCTGATGTGTGACACGCCTGCCTAACTAGAGAAGAGAGCACTGGGTAGAGGTGGACACAGGTGTGGCTGCCCTC
CCCAGTATCACTGTCCCATGGCGAGAGGTCAGAAAGGCAAACAAACAATGGGGGTAGATGCTGAGCAGGGAGGGGCCCTGAAGCAGGACCTGGGGACA
GCCAAGGACAACTATTTTGTGAGAGAGGAATGAAACCTTGCAGGTCCTGCCACAGAAGCAAGAAGCAGAGGAAAGGCCATGGAGAGACTTAATAAAGG
GTTTTACAAGGGA
MOUSE SEQUENCE—CODING
ATGGCGCAGGAGAACGCCGCTTTCTCTCCCGGGTCGGAGGAGCCGCCACGCCGCCGCGGTCGCCAGCGCTACGTGGAGAAGGACGGTCGCTGTAACGT
GCAGCAGGGCAACGTCCGCGAGACCTACCGCTACCTGACCGACCTGTTCACCACGCTGGTGGACCTGCAGTGGCGCCTCAGACTGCTCTTCTTCGTGC
TCGCCTACGCGCTCACTTGGCTCTTCTTCGGTGTCATCTGGTGGCTCATCGCCTACGGTCGCGGCGACCTGGAGCACCTGGAGGACACCGCGTGGACC
CCGTGCGTCAACAACCTCAACGGCTTCGTGGCCGCCTTCCTCTTCTCCATCGAGACGGAGACCACCATCGGCTATGGGCACCGCGTCATCACCGACCA
GTGTCCCGAGGGCATCGTGCTGCTGCTGCTGCAGGCTATCCTGGGCTCCATGGTGAACGCTTTCATGGTGGGCTGCATGTTCGTCAAGATCTCGCAGC
CCAACAAGCGCGCCGCCACTCTCGTCTTCTCCTCGCACGCCGTGGTGTCTCTGCGCGACGGGCGCCTCTGTCTCATGTTTCGCGTGGGCGACCTGCGA
TCCTCACACATCGTCGAGGCCTCCATCCGAGCCAAGCTCATCCGCTCCCGTCAGACGCTCGAGGGCGAGTTCATCCCTTTGCACCAGACCGACCTCAG
CGTGGGCTTTGACACGGGGGACGACCGCCTCTTTCTCGTCTCACCTCTCGTCATCAGCCACGAAATCGATGCCGCCAGCCCCTTCTGGGAGGCATCGC
GCCGCGCCCTCGAGAGGGACGACTTCGAGATCGTAGTCATTCTCGAGGGCATGGTGGAGGCCACGGGAATGACGTGCCAAGCTCGAAGCTCGTACCTG
GTGGATGAAGTGTTGTGGGGACACCGGTTCACATCCGTGCTAACCCTGGAGGATGGTTTCTATGAGGTGGACTACGCCAGCTTCCACGAAACCTTTGA
GGTGCCCACACCCTCGTGCAGTGCTCGGGAACTGGCAGAAGCCGCGGCCCGCCTTGATGCCCATCTCTACTGGTCCATCCCCAGCAGGCTGGATGAGA
AGGTGGAGGAAGAAGGGGCTGGGGAGGGGGGCAGGTGCGGGAGATGGAGCTGA
HUMAN SEQUENCE—GENOMIC
GGAGTAGGAGAAAGCTATGGCATTTTAGGAAAATTAATCGGGAGGTGACAAAATAGTTTGAACCAAGTGGATATAGTAGGCAAGTAGACGATAGAAAA
TAATTGCAATAATATAAGCATGAAGAGATGACAGCCCAAATCAGCGTGGCAATGGTGAAAAGTGGAACACAGAAAATGAATTGGAGTACAGAAAAATC
AAAAGAAAATGAAAAAAGTTTGAAGCCAACTTGACATGTTGAGCAAAAGAGGGAAGCTTCAGAGATCATACTAGAGTCTCAAGTCAGGTGATCAGAAC
TGCGAGGTCATTCACGGGCATAGGGGAGCCTGGAAGAAATCACACCTGGTGAGGAGACTGAGGTGGGGGAAGAGGAAGTGATGAGTTCAGAGCTGGAA
GCTGTGGAGAGGGGTCAGAACCAGAGAGAGAAAGGAGGTCATTGCTGCCAGGGCAGTGTGAGTTGAAGCTATGAGAACAGGGTAGATCCCAACAAAGA
CTGCACAGAGAAATGAGAGCCTGGCACAGAGAGTGAGGAACACCTATGTTTAGGGGATGGGAAGAAGAAGGACCCCCAAAGAGTGAAAGAGAATCCAC
CAGACAGGCAGGAAGGAGACAAAGAAAGTGAGATGTCATGGAGCTAAGGAAGGAGAGACTGTTAAGGAGGAGGTTCTAACAGTGCCAACAAGTACAGA
GAGAAGAGGCATTGGGTTTGGCAGTGACAAAGTCTCTAGTGACATTTGAGAGCAATTTCAGAAGAGTGAGCAAGGTGGGAACCAGATTACAAGTTACC
ACTAGAAAGTGAGAAACTGTCAGCAAGTATAGGTTACACTTTTGAGAACTCTACTCATAGAGAGGAGAGAAATAGAAACCAGACAATGTACTAGAAAC
AGGCCAGGCCAGGTGGCTCATGCCTGTAATCCCAGCACTTTGGGAGGCCAAGGTGGGTGGATCACCTGAGGTCAGGAGTTTGAGACCAGCCTGGCCAA
CATGGCAAAAACCCATCTCTACTAAAAATACAAAAATTAGCCGGGCCTGGTGGCACGCGCCTGTAATCCCAGCTACTTGGGAGACTGAGGCATGTGAA
TCGCTTGAACCTGGGAGGCAGAGGTTGCAGTGAGCCGAGATCACGCCACTGCACTCCAGCCTGGGGGACAGAGCTAGACTCTGTCTCCAAAAACAAAA
CAAAACAAAACACAAAATAAAAAGAAAAAGAAACAGTCTTCCAGTTTTTCTTCTTCACACTCCGAATGCCCTCTCTTCCTAAGTCAATATGGATGAGG
GGCTGTGGTGAGGTGGTCTGAGGGCCAGCCTGCAAGACTGGTATAAGACCTTTAAGTTTCAAAAAATAGGACATCCAAAAGATCCTTAAGGGGGCCAC
AGTCTTGACATTCACAGACAGAGAGGACTTAGGCAGGGGTGTCCAATTTTTTGGTTTCCCTGGGCCACATTGGAAGAAGAAGAATTGTCTTGGGCCAC
ACATAAAATACACTAACACTAACAGTAGCTGATGAGCTTTAAAAAAAAAAAATCACAAAAAAAACCCTCATGATGTTTTAAGAAAGTTTACAAATTTG
TGTTGGGCCTCATTCAAAGCTGTCCTGGGCTGCATGCAGCCCACAGGCGGTGGGTTGGACAAGCTTAGCTTGGAGGCTCTGGTGGAACTCCAAAATAA
ACATGAAGAACACCACAGAAGAGAAAGCAAAGGGACTGTAATGATTTATGGATCATTAACAGACATTTATTGTGCACTTATTATTTTTCCAAAAATGT
TATCCATCCATTTAGCTTCAACTACCACCCATGTGTCAATATGTCCAGCCCACCCGGATATCCATTTCAAACTCAACATATTTAAAGTCGAACATGTC
ACCTTGCTCACAAGAGTGCTCCTCTCCATTTATTCTCTACCATGGTAGATACACTATCATCACCCAACCAGAAACATGGCAGCCATCCTAGATTCTTC
AATCTTCCTCACCTCATCTCCCTTATTGAATCAATGCATCTGTATTCTAAATAGCCTCAATATTGTCCCCTTCCTCTCTATTCCACTATCATTGCTGT
AGTCTAGGACACCATTACCTCTCACCAGGTAATAATAGTTTGGATCTTTGATCCTGCTCAAATTTCATGCTGGATTTTAATGCCAATGCTGGACATGG
GCCTGGTGGGAGGTGTTTTGATCATGGGGGCAGGTCCCTCAGCGGCTTGGTGCTATCTTCATGATAGTGAGTTCTTGTGAGATCTGGGTGTTTAAAAG
TATATGGCAACATCCCCCATCATCAACTCTCTCTTGCTCCTGCTTTTGCCATGTGATGTGCCTGCTCCTGCTTTGCCTTCCACCACGAGTAAAAGCTT
CCTGAGGTCTCCTGAGAAGCTGAGCAGATGTCAGCACCATGCTTCCTGTAAATCCTGCAGAACTGTGTGCCAATTAAACCTTTTTTCTTTATAAATTA
CCCACTCTCTGGGTTTTTTTTTTTTTAATTTTTAATCTTTTTTTTTTTTTTGAGACAGGGTTTCACTCTGTCCCCTAAGCTGGAGTGCAGTGGTGTGA
TCACAGCTCACTTGTACCCCTGAACTCCTGTGCTGAAGTAGTCTTCCTGCCTCAACCTCAAACGTAGCTGGAACTACAGGTGTTCACCATTACACCCA
GCTATTTTTTTTTTTTTTTAACTTTCAGTAGAGACGAAGAATCGCTATGTAGATCACGATGGTCTTGAACTTGTGAGCTCAAGCAGTCCTCCCACCTC
AGCCTCCCAAAATGCTGGATTACAGGCTTGAGCCACCATGGCCTATCTCAGGTATTTCATTATAGCAATGCAAGAATGGCCTAATACACCAGGGCTAC
TGCAGCAGCCTTCTAACTACTCTCCCTGCCTCCAGTCTTCCTCCACTCTAATAATTCTTTGGATTATGAATTTCTTTATTTGAAAGTAATTAAGCACC
AGTAAAGTACATCTCTCTGAAACACACATCTGACCGTACCACTTCCAAGTTTTAAAACCTTCAGTAACTGCCAACTATCTATAAGTAAAGTCCGAGTT
CCTTTCCCTGGAAGAGAAGGCCTATTATAACCTGGACCTGGTGCCATTCCAGCCTTATCTTCTTCCACTGCCCCTATACACCCAAAGCTACAGCTACT
TCTTTTAACACTCAAGGTTCAGCCTTATGTTCTCTTTCTGTGTCTTGCCCCTTGAGCCTTTGTCATTTACATAGCTCCAACGATTGTCCCTGAGTGAT
GCCCAAATCTCTATCTTCAATCCTACACTCTCTTTGGAGCTCCATATTTCTAGTTGCTTGCAGGGGATTTCCATCTTGACTTGACAGGCCCCAAACTG
AACTCAGTACCTTCCTCCCCAAAGGTGGAAGTGCTCGTGACTTCCTTAGTTCTGTGTTACTCCTGGTCAATTAGAATAAAAAACTACAAGTGACCTTT
ACTCTTCACCGTTGCCTTGGGCCCATTCCTGGACATGTCAAATAAGCCAACAAATGCTGTCAAGTCTCCCTTTCTTTCATCTGTTTGCAATGTGCTTT
TTCATTCCTATGACCACTATCAGAATCAGAAAGATCAGGACCTGACCCATCCTTCACCTTTCTCTCCAGTCTCAAAGGAAGAGGCAACCTGTTTTCAT
CATGTCGGTCCCTGTGCCCTTGTTTTAAAACCCCAAACAGTTGCCTCGTGCTTACAGGTCACAGTGAAGGAGGTCTTCACCACAGAAGACCTAGAAAA
AAAAAAAAGAAGATAAAAAACGTGACAGGCCCTCAGACTGAACTCGGCATCTTTCTCTCTGAGGCTCGAAGTGCTAATGACTTCCTTAATTCTATGTT
ACTTCTGGTCAATCAGACTAAAAAACTACAAGTGATCTACAGAAGTGTCCTCTACTAACAATCAGAGTGAGGATAGAGTCGGGTGGGACTGGGCAGTT
AGAAAGACTTTATAAGTCCTTGAACAGCAGGGGTGGGAGCTTGTGGAAAAGTACACAGGTAGCTTCAACAGCACTGTAATGTTCTGAATTTAAAAGAG
TGACTTAAATTGAGTTTTTGTTCTTAAATTATGCTTTATAACATATAGACATATGTCCACCATCTATATTCTTTTGTACATATCAAATGTCAGGTTTT
CATTTTTAAATTTGTTTGCAAAAGAGAAGTCCTAGGACAGTCTCTAGGAGCCCAGTAGGGAATCAGTAATAAGGGGCATAGGACACTAATATTTGTGA
GTGTTTACTACATCAGATAGATCAGAAGATGGGGAAACTGAAGTTCTGAGGAGTTAAGTGGTTTGCCTATGGTAACATAGCTGGAAAGTGTTTTGAGA
TTTGAATCCACATATATTTGACCCCAAAGCTTGTCTCAGAATAATGCCAGAGGGAATTTGCACGTTTTAAACACAATCTTGCCAACCAGAGGCTATGA
CCCCTGAGTACATGTTGGTATGAAAAATTCCCCAGAATTACAACATCCAATGTCCACCATGAAACATGACAGAGGAAACTTCTCTTTTTGAAGACCCC
TCTCTCTTCTTTCAGTTTCCCAACTTGCGTCTTCCTTATTCTCCTCCATTTCTCCTTTCAGACTCACTGCTTCCAGCTTTGGCCTCATCTCTACTTTT
ACTTCATTTGTAATGGGGCAGAGGCTACCTCAGAGCAGAGGAGGAGGAGAGTTGGGGCGTGTCACCTGTTTTAGAAAGAATCCACAAGTGGGCAGCAG
TCTGAGGGGCTTGCGCTGGGCAAAGCAGATGTGGACAGAGGGAATCAGGAAAGCTTTGGGTTGGGAGGCATGATAGAGACTCAGAATAGTCAGTATTT
AACAAGTCAGGGGAAGTGGCTAGAAAGAACAGAGACACTGGCATGGCTCACCACAGGATTCAGGATTCCAAGTGGCGTTTTGGTGCTCACATCCCACA
GTGCGGAACAAATTCCATTAGTAGTGGAGCATCTCATAGCTGAATGACTCAGGCCGCAGAGGAGAAATCCAAGAGAAGGACTGAGCTACATTCCCCTA
GTCACTAACGAATCATTATGTAAGTAGATCACCCCCTTTAAATAAATGCAATATACACAAACCCACATTTATAAGACATAATTTAGGGAATACTTAGT
TACCTACTAAAGAATTCTTTCCTTTAAAAAAGAAAAACATGGCCAGGCACGGTGGCTCATGCCTGTAATCCCAGCACTTTGGGAAGCCAAGGTGGGTG
GATCACCTAAGGTCAGGAGTTCAAGACCAGCCTGGCCAACATGGTGAAACCCCATCTCTACTGAAAATACAAAAAAAAAAAAAAAATTAGCCAGGCAT
AGTGGTGTGTGCCTGTAATCTCAGCTACTCAGGAGGGCGAGGCAAGAGAATCACTTGAATCCGGGCGGTGGGGGTTGCAGTGAGCGGAGATCGCGCCA
CTGCACTCCAGCCTGGGCAACAGAGCGGAACTCTGTCTCAAAAAGGAATAAAAAAAAAGGAAAAAAGAAAAAAACAAATTTCTCTAACTAGGGACTTC
TAGTACCTTTCCAGTTGGGTCCAATTGATAGAAATTCCATTAACATCCAATGCACTGTGATAGGAGGGAGGCAACTGGGAATAAAGAAACACGAGGAA
TCTCGAGTCGGGTGGCCTGAGTCTTAGTCCTGACTATGTTCTTGGGACCTATTCCTACCTGTAAAGTAAGGGCTAATCCTGTACCACCTCTAACCGTC
ATATAACTTTTAAATCTTAGCCTATCTCTACCCAGTCCTATAAAGCAAGATAGAACTCTGTGTGAAGGCTTCTGATCCTCCTGCTCTGCTGAAAGTAG
CCAGAAAGGCAGCAAGCTCCTCAGCCTCAGGAACCCAGCCTGAGGCGAGGGGCTGGCTGAAATTGCCTCCGTCTGGCCTGGAGCTGTGCTCTGCTTCT
CCCCATTTCACTCTATCTTCAGCTTCAGTCATTTGCCAAATCTACTCCTTCAACCATATCTTTCCTCTGCTCTGAGTTTTCTAGAGCCCCACTCCCCC
TTGAATTTATACAAATTTTTGCAATCAACCAGATTGGCCTCCCTGCTCCACTAAACTCATATCCTCAACTGTCTGCTGTCTTCCCCATCATGCTTCCT
CTTCCACTTGCCAGATTTTGCACAAGATGTCTCATGATCTTGTCCAGGGAAGCCTCCCCAGTTTGGCTGATCTCAGGGCTGCCACAAAGGCACCTGCT
GATGGGGCAAGTTGAGGACTGAACTGCATTCAGCTTGCCAATTCCTGCACCCAGCTCAGAGCTGTGTCTGCTGGAGGAAGGGAACCTTTTATTTTCTC
CCAAAAGTATCACCTGTTCCCTGTTCTCCAAGTGACAGGCCACAGTAGGCTCTTTTTAAGCTCTTTTCCTATTTTGCACCACGGTTCCCTTTTTTTTT
TTTTTTTTTTTTTTTTTTTTTTTATGAGACAAGGTCTCACTCTGTTGTCCAGGCTGGAGTGCAGTGGCGCAATCACGGCTCACTGCAGCCTTGAGCTC
CCAGGCTCAGGTGATCCTCCCACCTGACCTCCAAGGTGGCTGGGACCACATGCACATACCACTACACCCATCTAATTTTGTATTTTTTTGTAGAGACA
GGGTTTCGCCATGTTGCCCAGGCTGGTCTCCATCTCCTGGGTTCAAGCGATCCGTGCACCTCAGCCTCCCAAAGTGCTGGGATTATAGGTTCGAGCCA
CCGTGCCAAGCCAAAAGCTAGAATCTTGTCTATGCTTTTGTGTCCTGGTGCCTGGGAAAACTTTTTTTCTCCTGCCTCAGTTCAGCTCAGTGATAAAT
AAGGAACTGAGGTTAGATAACAGGTAAAGTCTAGGACCTGCAGGATGAGTGAATCAGGTGAGGGAGTGGTAGTCTTCTTCCTTGTCAGCCAGGCTAGG
TTCAGGGGCACCTGGACTGAGGCGAGGGGCTGGCTGAAATTGCCTTGTGGAGGGCCCTGCCAGTGATGCCCCCTCCAGCAAATAGGGCCAGCTCTATG
CAAATGTGTTCTTGCCCAGGAGTTTGGTTTCTTCTCTCTGAGCTCCTGGCACAGTGGAACCAATGTGAGCAGCTGCTTGGCAGGACAGAGAAGGGCAG
GCTAGCAGTCCCAAAGCTCGGGTGACAGGACCAGGCCCAGGAGACGGGGATGTTGACTGGGGCTTTAACAGCACTCTTGATGCCAATCTCGGGCTGAA
AACTCGATATTTCCACTTGGAACAACAAGAATCACCAGCAAGAGAGCTGACGAGAGGGCAGTATACCGGGGGCGCCCCCTGCAGGCCTCACAGGGTGG
TGCCAGAACAGAGGAAGGTGGCACAGGCAGGGTGGGGCTTTCAGGACATCCCTGAGATGATGGTGATGACGGTGACAATGATGATGACCATGAAGAAG
ACAATGAGGAGGAGGAAGAGGAAGACAGTAGCTAGCATTTACTGAGTACTAACAATGTGTCAGGCATTGCCTTATGTAGTCTTCATGACAACCCTCTA
AGAGATGAATAATATGGTTTTCTTTTTTTTAGATGAAGAATCTGAGGTTTAACGGGTTAAATAATTGCTCAGGTTCACCCAGCTAGTAGTGGACAGAG
GTGGGAATTTGAACCCAGTCATTCCCTCCTGAGCTTATATTATCAAGTACCGAATTTCCCACCTTGCCAAATCATTCCAGGAGCTTCTAGCCCTCCGT
GTCCATCTCTATGTCTTCCTGCTCCTCTAGCTCATATTTTCTTGATCCAAATTTAAAGGATCTGGATAAGAATAGATCCATATCTGGGATATAATAAT
ACTGATAACAACAGCAACAACACTTTGCGTTTGTAAACCACTTTCTTCTCTTCATTATTTCCCTGGGGAAAAATAAACAATAAGATATTTCTGTTTCT
CCAAATTTTGTTCTGATTTTTATCAGTGTTCCTGAAGCTATTTCAATATAGTCATGATCAATTTCTAAGAATATTTTTAGGTTCTGCTTTTTTATGTA
ACAGTGTGTTATATACACATTCACATATTTAAACACAGCAATTATTATGGCTTTACAGTAACCCATGATATTAATATTCCACAGATATTACATTACTG
AGGCACACTAGGCTAAGGCTGACAACACCAAATGCTGGCAGGAATGTGGAGCAACAGGAACAGGAATTCGTGGCTGATGGGAATGCAAAATGGTACAG
CTACTTTGGAAGAAAGTGTGGCAGTTTCCTAAAAAACTAAACATACTCTTACCATACGATCCAGGAATCATGCTCCTTGGTATCTACCCAAAGGAGAT
GAAAACTTACGTCCACATGAAAATCTGCCGATGGATGTTTATAGCAGCTGTATTCATCATGGCCAAATCTTGAAAGCAACCGAGATGTCCTTCAGTAG
GTAAATAGATAAATAAACCATGGCCATCCTGAAATGGAATATTATTCAGTGCCAAAAAGAAATGAACTACAAAGCCATGAAAAGACATGGAGGAACCT
TAAATTTACATTACTAAGTGAAAGAAGCCAATTTGAAATGGCTACATATTGTACAATTCCAACGACATGACATTCTGGAAAAGGAAAATTATGGAAAC
AGGAAAAAGAGCAGTTGTTGCCAGGGGTTAGGGAAGGGGGATTGACTAGGCAGAGCATAGAGGACTTTTAAAGCAATGAGACTATAATGGTGGATACA
CATCATTATATATTTGTCCAAACCCACAGAGTGTACAACACCAAAAGTGAACCCTAATGTCAGCTATGGACTTTGGGCGATTATGATGTCAATGTAGC
TTCATCACTTGTAACAAATGTGCCATTCTGGTGGATGTGTGGGGACAGGGGGCATACGGAAAATCTCTATAACTTTCCTCTCAGTTTTGCTCTGAATC
TAAAACTACCCTTTAAGAAGTCTTCTTTTAAAACAATTTACAAAGCATGAGGTGATACAGATGTGGGAGTTTGGCTCCTGTCTCTGCCCAACTCTGTG
ACATTCGATAAATTACTTAACATGTCTCTGTTTCAGTTTCCTCATCTATAACTGGGAAAAATAACACCTGTCTTATAGAGTTGCCATGGGGATGACAT
GAGGCATGTGTCTCGTTCATATCCCATGCTCAGTGAATTAGTAGCAGCAGCCACTGTGTGTTTGTGTGTCTTTATCCCTCCTGGGTTAATGAGCTCCT
TGTGGGCAGGGACTCACCCATTCTGTAACCACCCCATCTAACACACTGCCTGGCACTTGGGCTCCGCAGAAGTTTGCCGAGTGAATACTTAGTAAGCC
CTAACCTAGGCTTTTCTCTCTGGTGGACATTTTGGGTTGTTTCTAGGGTTTTTGCTATGAATAAAACACATTTCAAAGCCCTTTGTGGTTTTTTGGTT
TTTGTTTGTTTGTTTTTTCTTCGTTTGATCTGCTGACTCTGTGAAGCAGGCAGAAAGGGGATATTTGCTCTTGTCCACACCCTGGTACAGATGGAATA
ACTGTGGCTCAGGGAAGTGAAGTGACTCCTATGGGACACAGTGCAAATCAGTGGCAATAATTAGAACCCCTGACCCTGCCTCCCTTCCTTTAGTAGAT
CTATTTTCCTTCTAGCTACCGCCTTCTGGATCCATGGCCTCTCCAAAACTAGACCATGATGGTCAGCCTGACCTGAGAGCAGCACCTGCACGCAGAGA
CCCATGTTGAAGGTGGTGAGCTGCCAGCTACCAGATGGCCCTCTGAAACCCCAGGGAACCTAGCACCTTATTCTCAAATACATGAGGGCTTGTATTTT
CCCCCAGGAAGGAGCTTCTTAGGAAAGAGCCAGCGTCCCAGCTTTGTTTTTCTTTCTTCTTCTTTTTTTTTTTTTCCTATGAGGGGGTGAGGAGCCAA
GCTCTGAGTTGTCCAGGAGGAGGGACTTTGGCTAAAAATAGCTATGGCGTGTGGTTTGGATCAACCCCTAGTGGTACCCAGGACTGGGGAGGGGAGGG
GGATGCTCTGGAGCTGTCGCCAGACTGGTTGCCGTGGAAACAAGAGAGGAGCAGGGGAGCCTGGGAAGTAGGGATGACACAGATAGCAAGTCCTAGTC
AGAGCTGCCGCTACATTTAGGAGAAACACCGGTGTCTGCGCCTCCCACCCTTCGGGGGGCCCGTGGGGGGGCCGGTGTCAGGGGCATGGACGCCACCC
CCCAGGGGTCTCTGCTGCCGGCTACTCTCCTCTCCACGTGCTGTGAGTTGAGTTGCGGGGGACTTGGGGTTTGGGCCCCTATTTCCAAGGCAAGTGGG
GGTTTGGGAGGAGCTGGTTCTTGGGGGAGTTTTCACCAGGTCTCTCCTTCCAAAAAATGAGCCCCCTTACTCCCCAGCTCTCTAGAGGGAGGAAGAGG
GGCCCAGGAAAAGTGGTATTGCAATCTTCTGCAAAGGGGTCATAGCATGCACAAGAAATGAGGAGTAGGTTGGAGGAACTGAAATTCTTGGAGGGAAG
ATGGAGAAATCAAGTCCTTGATCTTGAGATAGAGGTAACAATTTCACACTTTTCCTTCCCCTGAGAAAAGTGCAGTCCCCCACTCAGGAAGACAGGAT
GTGGGACACATTCAAAATAAGGTTTACCTAGATCCCTGGGGCAATGGAGAGTGAGAGAGTTCTGGGGGTGATCCGACATCGGGGTTCCTTCCCCATCC
CTGGGCAGAGAGATCTGTCTAGGCAAGCCGACTGGGGGTCAGATTACCTAAGACCCTGAGAGAACATCTGGAAGCCCACCTGGGACTAAAGCTAGGAT
AATGGGAGCAGGGTCGTTTTCTGCATGACCTGGGGTCTCTGAGCCAGTCAATGCTTACTCTTCCTGAGGACATCTGAGCTTCAGGAAAGGAAAAGGAA
GCCCATTGTTGGGGGCAGGGGAAACCCTAATCTTCCATTGCCATGGGGCTCTTGGACCCTGTGTCCCCTGACTCCATGGACAATAAATGCAGGGGGTG
CCCCTAAGCTCAAAGCCATTTCATTTTGATTTCTCTTCCTACCTTCTCTACCCCAAGACACACAAACACACACACACACACCCTCTCCAGAGTGCTGA
CTGCAGAGGACCTCACCCCAGAACATAAGATGCTGGAGTGCTAGGTTTAGAGTCACATACCCAGGCAGTTTCTCCCCAGGACCTGGTCAACCATCCAG
GCCATCTGTGGTTCCTATGGCACACTCCTCCATCCCCCACCCACTAGCCAGCCCACGTTTCCGTGGAGTGGGAGGAGAGGATCATTCCCAGGAAAGAG
AAGGGAAGGTGGAAGAGTCCCAAATCCTATTCTAAACCTTTCCCTGTATGGTCCATATCTCCTAGAGGACCCTGGGTGCTTTGGGGAAGGGCTCTGGA
CCTCTCTCAGAGCAGATTGCAGCTCAGAGAGCTCCTCAGAGGCAAGCATGTGAAGAAAAATCAGGTGGGCTTCGCTTGGAATGTGGGCTTTGGGGCAT
ATGGCAGGTGGGGGCGGGGCTGGTGTTAGGATAGTCCATGGGAAGTAAGAGGCTGGGGGAAAATATAACTAGAGGGAGTGGGGAAATAAATGTGGGTG
CTTAGTGCTTCACCTGATCTGATTCCATGTCTCTCATGAAGAATAGGATCCCAGAGGGATACGAGCCTAACTCTTTATAACTCTGGGCTTCCTTTCCC
AGGCTTCTGTGTTGGGATCTTCCAGTTCCCCTCCCCATTTGCAGGCTGTCTCCACTAGGAGAAAAAACCCAAGGGAAATGAGGCTGGCCCAAGAGCAG
CAGTGATCGTGGGTAGGTCTCAGGGAGGATTTCTAGTGGGAATTTCCTAATGTTCCACCCTTGTGCACTGGAGGGTTTCCACTGACTTTCCACAGCTT
TCATTTCTTTCTCGTTTGTAAGCATGTTGAGGGGAGGGAATGGAGCGGAGTGAGTGAGGTCCAAGGAGGGAAGAATGAGAAAGACTGTGTATCAGTCT
TGGGGTGAACTTCAAAACAGCCTGCGAGGAGAGCCATTGGTGGCTGCACTGGCTACAGCTGGGGAAGGGATGGTGGAAGTCCTTAGGGCAGGGAGGGC
TCCATTACCCGCCTGCCCCCCTCCCCAAAAAGCCCCCAGTCTATTGATTTCAGGAAATCACTAGGGGGATCTGGGCCTGGGTCTTTGGCCCCGGGGCT
GCCCCTGAGGTGCTGCACACCCCAGCTGGAGGTGATGGCACCAAAATATCTGGTACCTCCTTCCCCTGAAAATCATCGTGGAACTTGCACAGTTCTAT
CCAGTTCAGGTACATCATTCCATTTGACCCTCACAACTTTCTGAGCCTGGGGGGCAGTTAGGGCTGAATGTGTTATTCCCAGAAATAGAGGCCAGGCA
ACACGAAGGGACTCGCCCAGGGCCCCCCAGGGCTCGGTGCTGGCCCTGATGCCCCGTGCCTCCCCATCTCCCGAGGGGCCACTCATTCGGCAAACCTT
TATTAAGCCCCTCCAGGACCCCCGACGCCGCCTAGGCGCCCAGCGACGCGCGGCAGGTGGCAGCAGCTCGGGCCCCCGCCGCACTCCAGGCGCCCGCA
GCGCTCGCCCTGACGCGGCCGCCATGGCGCAGGAGAACGCGGCCTTCTCGCCCGGGCAGGAGGAGCCGCCGCGGCGCCGCGGCCGCCAGCGCTACGTG
GAGAAGGATGGCCGGTGCAACGTGCAGCAGGGCAACGTGCGCGAGACATACCGCTACCTGACGGACCTGTTCACCACGCTGGTGGACCTGCAGTGGCG
CCTCAGCCTGTTGTTCTTCGTCCTGGCCTACGCGCTCACCTGGCTCTTCTTCGGCGCCATCTGGTGGCTGATCGCCTACGGCCGCGGCGACCTGGAGC
ACCTGGAGGACACCGCGTGGACGCCGTGCGTCAACAACCTCAACGGCTTCGTGGCCGCCTTCCTCTTCTCCATCGAGACCGAGACCACCATCGGCTAC
GGGCACCGCGTCATCACCGACCAGTGCCCCGAGGGCATCGTGCTGCTGCTGCTGCAGGCCATCCTGGGCTCCATGGTGAACGCCTTCATGGTGGGCTG
CATGTTCGTCAAGATCTCGCAGCCCAACAAGCGCGCAGCCACGCTCGTCTTCTCCTCGCACGCCGTGGTGTCGCTGCGCGACGGGCGCCTCTGCCTCA
TGTTCCGCGTGGGCGACTTGCGCTCCTCACACATAGTGGAGGCCTCCATCCGCGCCAAGCTCATCCGCTCGCGCCAGACGCTGGAGGGCGAGTTCATC
CCGCTGCACCAGACCGACCTCAGCGTGGGCTTCGACACGGGAGACGACCGCCTCTTCCTCGTCTCGCCGCTGGTTATCAGCCACGAGATCGACGCCGC
CAGCCCCTTCTGGGAGGCGTCGCGCCGTGCCCTCGAGAGGGACGACTTCGAGATCGTCGTTATCCTCGAGGGCATGGTGGAAGCCACGGGTGCGAGCA
GGCCTGGGGAGGGGAGCGGGGTTGGCAGAGGGTGGGCGGGACCGAGGAAGGCAGGGGCGAGACTAGGGGCCAGGGGAGCTGGGGAGGATGGATGGAGG
GGCTGGTGGAGGATGAGACAGTGAGGTGAGACAGGGGTCGGAGGCGGGAGTGGAACCGAGCAACGCCGCAGAAGGCCAAGAGAAAGCTTGGAGGAATT
CTCCGAAATGGCACTGGCGTGGGGCCCTGGGCCCAGAGGAATGTGTCACTTGGAATAGGGACAGTAATAATAGCTAGTGCTCGCCCAGTATTCACCCT
GTGTCATGCGCAGTTCCAAAGCACTTTCTACCTCTGAGTCGATTTAATCCTAACAAGAACCCTCTGAAGGTAACTTCTTGTTATTGTGCTCACTTTTT
AGAGATGAGATTGCTCCAATGAGAAATTAAGGAAGTTGTCCACTTTCCTAAGCCAATAAGTGGCCATGCCTGGATTGGACACAGGCAATGTGGCCTCA
ATGTTTAGTGGTCCCGAGTTGGAAGGAGGGGTTAGGTTCAGGGGTTTTCTCACTGCAGTCAGGTTCAGGCCCCTGGAATTTGACGGTGAAGGTTTTCC
ATTGCCTGAGTTATTTCTAGGCCGGATCTTGAGGGCAGTTTAATACCTAGTCTAACTTGTACCTCGGTTTCCCAATTCATCCATTTCCACTGACAAGG
GATATAGATGATGTTACCTTTTCTAGCTCTTTTCCAAAAGGAACTGGCAACTCATCTGTGATGTCAATAAGTCCAACCCAGACCTACACAGTGAAGGC
TTTGGGAGCAGGTGAAAAAAGACCAGTGTTACAGGAGTCGCAAAGGAGGTCACTTAGGACTTGAGATCTAGAGGATAGATGAGGATGAGGAAACTGCG
GGTGGAGGACCAAAGGCCCACTAGGGGGCGCCGCAGTCCCTCCTCTGACGCCAGAGCTGCTGATGCTCCCTGCCGGCTTCGCTGACAAGCTGGTGCCT
TCAGATCCTTTCCCTGGCCCCTTTAGGCTGAGACTCCGCTTCACACCCCAACCCCAGCTCCGCATCACTGTTCCCATTCCTGCTTCACCCCGACTCTT
TCCTCTTCCCCCACTCACCCCGTTCCCTTTCCTCTCTCTCCAGCTGTCACTCCTTTTCTGCCAGTATCTCAGGCAGGCCCCTCACCCTCCAGGGAAGT
TGCTGCCCGGCCCTCTTTTCTCTTTGTACCCCCAGCCCTGCCCTCTCCTCCTCGAAGCCCTTCTCTCCCCAGTGTCCCTTATGCCTCTTTCTCTTCTC
TCCCACTGGATACTTTCTATTCCAACTTCACCGAGGAATACCAATGTCTCAGCGCCAGGCTTTCCGAGTTGACAGCCACTCTCCGGTTAGCTAATGTT
CACTCTTCTGTTTCCCCTTGTTCCGAGATGGATATGGGTTGGGGGCAAGACCCTGTGGCAGAAAGGAGAATGACCTGCCCTGAGGGGTGCACCAGCCC
AACAGGAAGATAGCACACAAGCCCCGGGCAGGGAGGACCAGGACAGAGGAGATGAGGATAGGAATCTGTCTGTTTTTCTAGAGAGATAAGCTGCAAAA
GGATGGTAATATTTTGGGTGAGACAGTCAGGATTCAAAACGCTTTTGAAAAGCAAGAATAATGAGCCAAAACCCAGCAAGATGACATTTAAAATGAAT
AAATATAAAATTCTACATTTAGGCTTTAAAAAAATCACTTATGTAAGCACAGCATGGAAGAGCACTGGTGAAAAAAGAACTGGGAGTTTTAGTTGGCT
ACAGTCTTGATGTCGTACCAATGTGATGCAGCCTCCAAAATCATTATGTAATGTTATCCTGGGCCCTATTAGTGAAAGCATAATGGCCAGAAGAGAGA
GATGGTGCGCGCTCTCTTATGCACGGAGCAGGCCACAGTTGGAAAATTTACTATACTCAAAATGCTTAAAGGGCCCTCCTTGGCCATTCTGGCTTGTA
ATCAAAAAAGTAGAGTTCTGGAAAACCAGGTCAAATGAGGAATCGTGGAGGAAGCCAGGGATGTTAAGTCAAGAGAGAAAACATGAGGGAATCTGAGA
CTCCTGTTTTCAGATACTCAGAGGACTGTGAAGTGGGAGGGGAATGAAGCCAAGAGTTGGAAATCCCAGGGTACAGGTTTTAGCTCTGTATAAAGAAC
AACCCAACTATTAGAGCTATCATACAAAGGAGTGGGCCCTTTATGAAGTGGTGAGCTATCAATCCTGGGAGGTAATCAAGTATAAGCTAGATGCCCAT
TGTTACAAATGCTCCTTTGGGGAGCCCTGTATGGAGTGAGAAGTTGGACTAGAGGATCCCTAAGGTTACTTTCAAGGTTAAGCTTTTTTTGGTTGGCA
TCACCAAATGACAGGAGGGGAAAAAAGAGCTGGACATTAAGAGGAGTTGGGGCAAATGGAGAAGACACGAGGGAGCTGGGTAAGAACAGGAGCTAGGG
AGGGGGGGAAATGGACTGGACCAAAGGGAGGTGGGAGCCCTTAGGAAGGAATAGAAGGGAGGGTGCTGGGAGTAGGGTTGTGGAATGAGAAGAGGAGA
GGGAAGCCTGGAGCTGAGATTCCCCCTGACCGGTGCCCCTCCTCCCAGGAATGACATGCCAAGCTCGGAGCTCCTACCTGGTAGACGAGGTGCTGTGG
GGCCACCGCTTCACGTCAGTGCTGACTCTGGAGGACGGCTTCTACGAAGTGGACTATGCCAGCTTTCACGAGACTTTTGAGGTGCCCACACCTTCGTG
CAGTGCTCGAGAGCTGGCAGAGGCTGCCGCCCGCCTTGATGCCCATCTCTACTGGTCCATCCCCAGCCGGCTGGATGAGAAGGTGGAGGAGGAGGGGG
CGGGGGAGGGGGCGGGTGGGGAAGCTCGGGCTGACAAGGAGCAGAATGGCTGCCTGCCACCCCAAGAGAGTGAGTCCAAGGTGTGACCAGCTTCCTCC
AGACCCCTGTGGCAGACCGGGGGCCAGACACAGATACATGGCGAACTGAATATCGGAGGTGGTCGAGGAGGAGGAGGAGGAGGAAGGCAAAGCCCCTG
GAAATGTGCTAAAGTTGGAAAGTCCCCGTCCCCCAGAACCTCAAGTCTAGAAACCAGTATGGAAGGGAGGGGTCCTGATTTCAGGGAAATGGAGGGTG
GGGCCGGGTGAAATGCCAGTCTGTGTTTGACCTTCACATTTGAATCATGAGTGGATGGATGGACAGAATGATGGACTTTTGGGGGTTGGATGGGAAGA
TGGTAGCAGATAAAGACAGCTGACAGATACATAGATGGACCAGTAGACAACTGGTCCACTCAGGGCTGCCACTAACCTGTAGAACACCCCTGTGCAAA
TTTTAAAAAGGAACCCTTTTCCTCCAGACAGATACAGCCCCAAACCAGGGTGCATGGCTTGGAAAGCAGAGTATAGGATGGATTGCAGTCCCCAGTCA
CCTCTTCTGCCAGCCTCCCCACATATGGCACAACTGTCTAATGACACGGTAGGCCAAGCTGAAGTGAAGGAGAAAGGAGCCGGACCAAGATGGGCACA
TGAGGAGGGTGCCCTCCTAGCTCCACCCTCACCAGGATGAAGGCGTGCAAGGGGCTCAGCAAGGTGTGAATGACCTTAGTCCGCAAGTTCAGGGAAGC
AGGCAGAGCGGGGAGGTGCCTGAGCTGGGGCCTGGAGAGGGGCCTGGGAAAGGAAAACCAGGGATAGCTATTTTCTTACAGTGGAGTGAGATCTTACA
GGTATCAGGCACAGGCAGGAAGAGAGAGAGAGAGGTTCTGGGGAGGAAGGGCCAGGAGAGAGATCTAGAAAGTGGGTTCACTAGAGCTGGGAAACAGG
GAGCCCCTAGGAAAGCAGTGTGTCCTTGGGGCACAGTCATTCACATCACTGATTGGGTGCCATGTGGAGTGGACATTCAAAAACCTGGTTCCTGTCCT
CAAAATAAGGGGCACCTGGGAAAACAGAGGAATCTACCTGTGGTGACTGAACGAGGGATAATTCAAACTGACAACCTGTGCAGTCCCGTGGAGGGTAG
GGGAGTGTGGGTGATCAGAAGGCTGGGGCCAGTGTAAGGCATAGGGAATATGTAAGTCAGGAGTTAGAAATCTCCAGTGTGCGTTGGAATCACCTGGA
GGGCTTGGTAAAACACAGATTTTTGGGCTCCACTCCAAGGGTTTCTGACCCAAGAGGTGGGGACCAAAACCATGCATTCCTAAGAAGTCCCCAGGTCA
TGCTCCTGTTGCTGGACTGAGGACCACACTTTGAGAACCTGTGCTCTAAGTGAATACTTGGAAGTCGTTTCAGGACATGGGGCATAGAAACTGAGGAG
TAGCTGAGAGGAAGATGAAGAGAACCTGAGAAGAAGCTGAGGATCCTCACAGGAGCAGACAGAGAAATGTGAAGCGTGGGGTTTTATGTGTGGGAAAG
GGACCCGAAGCCCAGGCTGAAGAGTTTAACTTTGGGCCCAGAAACTCAACCATCAATGGAAACAGGGCAGTGACAAGTGGAGGGGGTGTCTGGAAGCT
GAGCAGGCCCGACAGAGAGATGAAGCCATCAGAAGGACTTGAGGGGGCTCCTGGCGAGGTCGGGGGGAGGTGGACAAGGAAGAGTTTTAAAGGCAAAG
GACAGAACCCCTTGTAGGACTGGAGGCAAGATTGAATGTGGGAGAAAATCGGAGAGAAGCGATAGGAGTTAGAACATCTGGATGTGTCTGCAGCCTGC
TGTCAGCCCAATTGGGCCAGGGGGTCCCAAAGACGCATATTCTCACCCCACCTCCACCTGCTTCCTGATCACATCCCAGTCACCAGCGGCAGCTTCCT
GGATAGTGAGGGAGAACAACTGCAAGTTGAGAGAGGCAGAGGGGTGGAAGGGACCTGAAGCTGGCCTGGAGAAAAGCATAGGCCCAGGAGAGCCTGCC
CTGGGACAGCGCCTGTCTCCCACACAGCAGCACTGGCCCAGCAAGGACCTCCTCCCTTGGCCCTGGCCACATCCCACTCCTGCCCTTTCATAAGCCCC
CTGGGGAAGCACTCCAGTCTTCTCTGTTCCAGAACTGGGCAGATAGGGTCCTATGGGGCAAAGCCAGGGTCCTATGGGCATAGCCAGGGCCCTATGGG
TCCTCTGGAAGCAAGAAAGGGGGCCATGGAAGCAGCCCAGACAGCTGGGGTTCACTCAGAGAGGACCCAAGTCCCAGTCCCTTCCTTTCAGTCAAAAC
ACGGATATCTTTGCCTCAGGTCACAGGGCCACTGGGGCCCTGTCATCAAAGATGAGATTCCTGAAGCCTGGCATTGACTGGTCCCCTAAGAACAGATG
TTGGGATGGAGAATGGGGATTCATTTGGGTTTCAGTAAAACAGGGGGGTCTGGACAAGAGCGGGTGGGCTACTTGGTATCCACACACACGCACTCACA
CAGGAGCCAACCCATTGCAGCTGAACAAGCAGAGAAACTCAGTCTGGAAAGGCCCCTCCTGCCTGCTGAAGTCACTGAGACCCTGCCACACCTCTCCT
CGCCACTGTCACCACTCAGGGCACCACTGTACAGTGCAACAAGTCAGGAGACCTAGGTCCTACTCCTGACACTTGCTAATTAGCTCTATGACTCTGGG
CAAATCGCATATCTGGGCCTCAGTTTCCTCATCTGTAAAAATGACAGCAAACTCGTAATGCTCAATAAATGTTTAAATAACAACTGAAGGAGGCCTGC
CAGATGCCTCTTAAGGTGCCGTGCAGGTAAGAATTTTAGGATCAGAGAATCCTTAGGCAAGAAAATTCATGAAACTCCTGGGGCACTGGAGGAGGGGT
GAAGCTGAAGGGTGGGAGGGAGGAGACCCCAGGGTAGGTACAGGCAGGTGAAGCGGGTATATGCAGGTGTAGTGGGTATATGCGGGTAGAGGGTATAT
GCAGGTACAGCGAGTACATGTGGGTGCAATGGCTCTGTGGACACAAAGGCCCTCCCCTGACTGCCTGTTGTCCCAGCCTGAGTATCAGTTGTCTTCTG
AGGCTTCTATTCTGCTGCTATGGGTCAGAAGGAACAACAATTTCAGCCCCAGGGCCTAGTGGGAGGAGTCAGGTCCAAGACTAGCCTGACCAGGAGAA
TGAGACGTGGGAAGAGTTGGGGAAAGTCTGGGAAGCTCAGAAAAGGCACTGCCCCTGGAGGCCCATGCCCTTTAACATGGGAGAAGCTGGTGCGGGGG
TGACCACAGGCAGCTGGAACCTACCCTCCTTTTCTATGCTTCCCTCCCCAAGTAGGAGTCCAATCAGGAGTTGTCTCAGCCCCGACAGTTCAGGCTGC
AGATGGAACCCAGGTGTCCCCTCCTGGGGTGGGTGGCATGGCCCATGGAGGCCAGATGGTGTTTGTGGTGGGAAGAGAGGCCTGGGTCATCCAGAATA
GGTTGTCAATCCCCAACCACCTCCCTACTATGCACCCTGAGCGTTTTACAGTCTCATGGTAGGGAAGACACAGCCAAGCCTGCTTTTTATAAAACAAG
TTTATTCACATTTTAGAAAAACTAATTCCAGGACAGGAAATGGCCTCCCTATAGGATCCCTAAGAGATCAAGAACAGAAGGCCAGAGGGAGGGGCTTG
GGAGGGAAGGAGTGGGGAAGGGGAGGCACGTCTCCCATTCTGGGTAGTGGGAGGTCAAATAAATTAAAGGAAGAGTGGACAGAGGGAGAGGGTGTCCA
GGCAACCAGAGGAGGGCTTGGAGCTGGGCCGGAAGACAGTCGACACCTGCAAGACCTGAAAAGGGTGCCCGGTGTGGGCTAAGGACAGAGAGCCCTGA
GTGGGGCTCCCTCGCGGCCTCCACCCCTTAACAGGGCCCTGTGGATCTGAGCTGCCTACTCCTCCTCAAGGTGGGGCCTAAGAGGGAGCAGCTTGGTT
CAGGACTTGGGGGGTGGGAAGCCCAATGAAAACAAGGTTGGGGGTTCTTTTCCCTCACCTGGGGAGTAAGGGATCACCGTTTTCGAAGCCTCTTCATG
AAGCAGCAAGTGATGGTACCAAGGACAGTGGCACCAGTGACTAGGGCCACCCCTGTACCCACCAGCAGAGGCACAAATAGGGTGTCCAGGGCTGGGGG
AGAGAGGATGACTGTTCAGAGAGGATGCCATCATCCTCCACCCATAAACTTGCCTCTGCGCTTTCCCCATCAAGTTCTCTGAACCCACCTTCTCCATT
CACAGACACCCCCATCCCTGCCCACAGCCTGCCCCCTCAGCATGCAAGTCAGCATCAACCACAGAGGACCCCGTGCAGGTGGGCACTGCAGGGCTGGA
AGTTGGATTTTTTGAGACTTCATGTGACATAATGTGGAGGAGAGAGATACTAGCAGGAGGGTCAGAAGATGGGAAGGGAAGGCCAGTGGCAGAGGCCA
GGAGGAAGGCAGAGTGAGGAGGGTGGAGGGGGTGTCACTCACCATGCATGTAGGGGTAGACTGTAACAGGCCCTGAGCGGGCACTGCCCGCCTGGTAC
CAGCTGTAGTCGGCATCCTGCACCCAGGCGCTGGGGGCACAGTGGTACACGCCTTCATCCTCGGGCCCCAAGCTGTGTAGTCTCAGCCGATGGCTTCG
GGGCCCCACCAGCTCTACGCTGACAGGGCCTCCTCCAGGCCCGACTCCCAGCTCTGCCACACCATCCTGGCCTACGCCACCCACCAGCTGGGCAGGGA
CAGAGCTGAGCTCTCCGTCCTCTGGTCGCTCCACCCACCAGCTGGCGGCCAGCCGCAGTCCTGGGGGGCCACCCCGCACAGAGATGTTGCACAGCAGG
GAGGCAGTCTCCCCGCGGTACACTGTGCCTCCTGCTAGCCATGCCACAGCCTCCAGCACCACACCTGCAGAACAAAGGACATGGGGTCAGAGGGTGCA
GGGCCAGGGAGCATGGGGTTAGGGCTGCCGCCAAGCACCGCCCCAGGAAACTCAGGGTATTCCCACAATCTTGGTAGAAGAGGAGCGTGAGGCTGTGG
CCTGCAAACAGCTGACGGAGAGGGAGGGGTCATGGAAACAGAAGGAAAAGGGGTTGACAATCCTCGAACCCCGTCCAGGGCCCAGCCCCCTCTCACCT
TCCTCCCGCACATGTACAGGGAGAGGCCGGGAACGGGCACTGGCTGCTTCACGAAGCCGGGTCCCAGACCCTCGAACATAGGCTTTGCCGAGGCAGCG
GTAGGTGCCCGCATCACCAGGCCTGCCACCCTCTAGCCGTACCCCGTATGTTCTGGATGCCACCTTCTCCATGGCAATGTGTCGGCCCTCATAGCCAG
GGCCCAGGCTGCCCACACCCTCTCTGTCCAGCTGGGCTACCAGGCGGCCGCGCCCCAGGTGCCCCCGCAGGTGCCATCTCCCAACCTACAGAGTATGC
AGCATGACGGCCTGCTGGGGGAAGTGCCCCTGACACATTGCACAGCAGTTCCAAGGGCTCCCCTGGGCCGATCCGACGTTCACCAGGCCCCACTGTCA
CTGCCAGCTGGCTGGCTGAAACACAGGTAGGGGAAGAGGTGTCATGGAGGCAGGAGGGGACACAGAGGCACCCGATTCCCCAACTTCCTGTTTCCTAC
TTGACAGCAGCAACTTCAAAACCTCCTGTCTCCCCCTCACTAGGTATGACCATCTTTCTATTTACGGCCTTGAATCTCACCCCTCAGCATGCGCCTCC
TATCTCTATACCCAATTTCTGAGCAGAGAAAACCCATCAAGGGCCGGGGGAGAGAAATGCTAGCAAGGCTGCTCACTCTGTGGAAGATGAGTTCCTTG
GAGTCAGATGATGGCTATCTGGTACCCCCTGTGGCCACAGTGCCCACCAGGATACTGTCCCTCCCAGCTCCCACACTGAGATGTATAAGTGGCACTTA
CACAGCGTCTGCACATCCACGTGGGCCAGGACGGCCCTTTTCTCTGCAATCTGGGCCCAGCTGCCATCAGGATCCTGAATCCACTCAGCGGCAGTGCA
GTGGTAGGTGCCTGCGTCCCCTGCCTGGGCACCCCCTACTACCATGCCGTACCGATCGGTCCCTTCCTTGCCCAGACGAAGCTCCCCTGCAGCCAATC
GCTCAGCATAGGGAGCTCCAGCCTCCACGGCCAAGTCTGACCGGATTCCCACCACTTCCTGCAGAGTTGACCGCCCAACTGGTGCCTCGGGCACAGAT
CGCCCAAAGGACACTGCCAGGTGTGTGTGCTTCTGTGTGCTTGTCCTCGCCAGGCAGCCCAGTGCCACCTCCTCCCCCTCATGCACCGTCATGCGTGG
GGGTGAGGTTGGGGCCTGGCGGCCTCGGGGCCCTGGGGGGGCAGCAGACACCTGGAGGACATCTGGAAGAACTGGAGAGAACAGCTGGAGTGAGGGAG
GGCTGGGAGCTGGCAGCCCTTGTTACTGTTTCCTGTGTATAGCCTATCTCCCTAAATAAACTGTGAGCTCCCAGAGGGCAAAGATCGCATGTTGTATT
ATTTCTTCTGTACTCAGTGGTGCGAGGGCAGTACTCAAGGCACAGCACAGGCGCTCAATAAATACTTGTAGAATTTCATAGAACCAGCCCATCGCCTA
CTCACCCTTATGTTTGAGACTGACCTCTGTTTGAAATACTGAGAAAAGCGGCTCTTTCTTCTCAGAAGACAAAGAAACTTAAGAGAGTGAGAATGTCA
CATGGTCTAACTCCTTCCCTAACTCTACTCTCTTTCCCAGATCTGGGTCCTGTACTGTCCAGGAGTAGAGGCTATTCAACCCAACAGTCTTCTTCGTT
CTTGGGAATGGAAAGTGGACTGGACAACTTAAGGACATTTCTTCTCCCAGGAGGGGTCTTAATATGATAAGATGAGCACTGGCCTGGCTGACGAACTC
TGGGTTTGAGTCCCACATCAGCCACTGAGTTATTGGGTGACTTTGTGCAAATCACTTAACCTCTTTGGGCCTCAAGTTCCTTGGCTACAAAACCTAAG
GGGCAACTAGATAGGTCACTTGTGGCCTTGACTTTCTGCCTTGAGACGGTGTGTGGCTCCACCCCGTCCCACGGCCCAGTACCTCTCACCTCCACCTT
GCCGCTGTAGCTGCCCAGGTAGCGGGTATCAGTGGAGGGGGTGTGGCACTCATAAATGCCGGCATCCTGGGCCTGCAGGCGGGCAATCTTGAGCACCA
CGGCATCACCTTGTAGGCGCTGCACCTGCACCTCACCCGCCACCACTCGGGACTTGAAGACAGCATAGGAGAACTGGGTATCCTTGGTACTGACAATG
CCCAGTGCGTATCTGCGGCCTCGGCCCTATACAAAGACCACTCCAAGTTCTGCTGGGCAGGAACCCTCATAGCCGGTCACATTCCAGGAGATGGAGAC
AGCTGTGCCAGCCACGCGGTACAAGGGCCCCTCGGGGACCAGCACCTCCCGGGCCCAGCATCCCATTCCTGTAGGGAAAGGCAGAAGGAGTTGGAGAT
GCCTGGTTCCTCATTCCATGCCCTCTGCCGCCACAAGCACCATTCTTGATCTCTGCCTACAAAAGGAAAGGAGACCTGGGAAAGCTTGTCCACAGCTT
GGACCCTGTTCTGAGAATAGGAAAGGGATGCTGTGATATAAGACACCTGGATCTCAAGGAGGTGGCATGGGCCCAGGATTGCCTTGGCATCCAGATGC
ATCCCATTTCTGGCGGACTAGAAGCAGAGCACCTGAAGGCAGAAAGGAGTACATCTGATTCCTGACCTAACCAGGCCTTGGTTCCAACTCAACCTTGA
TCTGTCCCTGCCACTCACCCACCTCCATGTCTGCCATTCCTTCCTCAGCACCTGGCAAGGGGAGCCTTCTGGCTAGGGGACTCTGAGACTACATGTCC
CTCTCCTTTGCTTGAGGGGAGCTGGCAGTCTTGCTCAGAAGTGCTAGTTGGCTCAGCTGTGTCACCTGGGCGAGACAATGGAGCCAGTGACCCTAGCT
GGAAAGGGCACAGGCCCAGTCAGTTCTCACCACACAATGCCCTCCCCTCTCCAGCTGCGCCATGAGCTCACTGCTTCTCTCACCCCACAGGGCTGCCC
AGGCAGCTGGGGCTTCTGGGGCAAGATCCAGGCTCTGCCCTGGCCATTGGGGGCAGAAGATCCCCTCCTCCAGTGCCTGCCAACCTTCCGGGCTAGCC
CAGCAGATACAGAAGGTGCCTGCCCCAGTTCCTTAACAAAAGCCTTCATTTGCACATGGTATGCATTCATTTACATATATGGCTCTCTTTCTGTAGGG
AGGCACTAAATCCCCAGCTGCCCCTTCTCATCTCTCTCCCTTCAGAAAGGCCAAACCTCTCTTCTTCACCCTACTCCACCCCTATGCCCAACCCTACC
CCAGCAGATACTCCTGGCAGACTTAGAGGGCTTACCTCCTCCCTTCTTTCCTTCCATAGCTCCCACTAGATAAGATCACAGAACCTCAATGTAAAGAG
GGCTAGGCCACCCCTCCCCACCTCTCCCAATTTTACAGATGAGAAAGGTAAGGCAGGAAAAGTATAATATGTTAGCCAAGATCATGCTGTCCCTAGAT
GGCTTCCACACACTCCTCCAGAGGGGCAAAACCAGAGAGGAAGATGGGGAAACTCCAAGGCCAGGCCTGAAGGGACTGACCTCACCAACCAGAGTGTC
ACTTTTAGGCCTCCCAGGGGGATACCATGGACTTTCTGCAGGAGCTAGAGGAAAATGCCCAGGAGTCTGTGGTCAAACTCTACCCTCCAGCTTCTCTA
GAACGGCTCCTCTGAACTTCCCCACCCCTCCTTCTGGGCTCCTAGCCCCTTCCTTCATCCTCTGGCTGGGTCACAGGGAGAACTCATCGTCTGTTGTT
AAGGGCACAGCTGCCAGTCAGGAAGTGGGATTCCAGCACCATCCCCATGCCCAGCTGTGTGGCCTGGGATCCAGTCTCTTTCTGTCCTAGGCCTCAGT
TTCCACACTGGAGGAGAACTAAGAGCTCCAGCTCTGACCATGTGTGAGTGCGTATGTGACTCAGGAGAGCCCTGCCCCAGGCCAGGCAAGTTTCATAA
TCAGAGTGACGGTGGAGACAGCCAAGCTGACACCTTCCCTGACTGCCTCAGGGCAGACTGCTCAGAAGGCCCCCTCCCATTTTCCTGGCTCCACAACT
GCTGATGCTTGGAGATGCCCATGGGAAAGTCACCTCCACAGCCTTAGCAAATCAGTTCCCACACAGCTCTCTCTCCCCTCCTCTGTATCAGTCGCAGC
AAGGAAAGGGACAGCAAAGAGGCCTGCTTTGGAATCAGATCTGTGTTCAAATCCTAGCCCCAACACTCACTAAATGTGCTCTCTGGGGCAAGTTACTT
CATTTTCCTCATTTGTGAAATGAATGTAAGTGCCCACAGGCAGTGGGTGCTCACACCTCTGCGTGCTCCTTTTTCAAACACAGGCCAGCACTTCCCCA
CCTCCCTGGGCTCCTCCCTGCTCCATGCTGCCCACTGGGGAAAACACACCAAGTGCTAGGCAACCCAGGCCCCACAGCGCCTTCCTCTCTGTACATCC
TCCTGCCACCTGCCCAGGGACCAGGGAGAGGACTCATCCTAACCCTGCAGGGCCCAGGGACCTGCAGCAGGGGAAGGCTTTGCTTGGTGCCACTGTGG
AGCTCTGGTCTAGAAACAGGCAGCTGGGGCTACCTTCAGCCTCTGCCTTGACGACAGCACCTCTGAAGTCACCATCCCCACCCCCACGCTTCACTCTC
ATTTCAAGCGCTTCAGCCTCATCAACATCTGTACTGGCAGTTTCACTGTCTCCATGCCATACTCTTCCCCAGACCACCTCCTACAGGGAGCCCTCCAG
TTCAGGCCAAAAACAATTCCACTGTCATTATCCCCATGCATCCATGCAAGATTGGCCCAGAACACCCCACCATGAACACCCACCACAGCAGGCACAAG
GTGCTTGGAGATCCCAGGATCAGTCTCCATGGAACCTGGTTTCTCCTGAGGCAAGGAAGCTGGAACTAAGCGGTGTGAAAACTGATGGGTGGCTGCAG
AGCCAAGTGCCATTTGGGAGACAGGAAGAAGGGCAAAGAGGGACCCAACCCAGGGTGGAGATGGGGGTGAGAGAGGGAACTGCCCCCAGTTGATGAAG
TGCGTGGAGCGCAACTGGGAGAGACTTACTTCAAAGATCGTGGGCAGAACTGGCCTCTGGGCCTCCAGCCAACTCTGGGGCAATTATGAAGCTGGGCA
GGCACTGCCCTCGTAGGGCGGGCACCCAAGGCCAGGCCTGGAGCTGAGTGTGGGGCAGAAAGGAGTCGCAGCATTTGGTGCAGCGACCCCAGTACGTG
GGTATGCTAGCTGAGATGTGTGGCCTGCCCCGGGAGGCCCAGCAGTGCCTGCCGCAGCACCTTAGTGGGTCCTCTCTACGCCCCAGTCCCTGGCTTAG
AGCTGGGGAGCCTGCACTCTTCCCAAGACTGGCTCGGCGGACAGCCACAAACCGCAGCTGGACGCCGACCCCGGGGACGCTGGAGGTACCCCTGACGG
AGGAGGATGTGAGGAGCCCCGAAATCCTAGGGGGGTGCTGGATGGCAGGCACCTGCCCGGCAGGGCCGGGAACCGGAACGGGGGCCTGGCTTACCTAG
CATTAGCAGCAGCAGCAGCGGCAGCGAAGGCGGCAGCAGCGTGGGCCTGAGGGCGCCCATCCTGCGCGGCCAGCTCTGGGGAGGCTCCGGGGGATGGC
GCGGGTTCTGGGGGGCCGGAAGGGTGGGGGGCCCATGCCCAGGTTGAGGGCAGGAAGCGGGGCAGCGAGGCGTGGGTGCGCCGAGCGAGCTGAACTGG
AGCTGCCGAATCCCCTCCCTCCGCCCCTCCCGCTGCTTTCCCTCCAGCCCTCGGCAGTTCTGAAACCATTCTCGCCCCGGCCCGCCCCGGCACCGCCC
CTTCCACCGCCCCGTCTAGGCCCGCCAGGACTACAGTCGGACTCCAATCCTGGCTCCTCCCCGGGCCCCGGCCCCGCCCCAGTCCCAAGCCGCACCCC
TTCCCCGTCCCCGCAGGGCTAACGTCAGCCTCCAATCCTGGCTCCGCCCTGGACCCCGGCCTCGCCCCGCCCCTGGCCCTGGCTCCGCCCGAGGCCCC
CGCACGACTGAGCTAACTGCACCTCTGCGCATCGAAATTCCCACCCACCCTCGCACAGACCGCATTCCACCCCGCACCTGCCAGCCTTTCCTGGAGAG
TTGGGTGCAGGGTCCCTGGGATTGGCCAGGTGACTGTGACCACGCATTTAGAATTCAGTTATTTGCTCTGAGCCATAGTCCTCGCTGCAAACCCTGCT
GAAGTAGGGGTTGGCGGAAGCCAGGAGTTCCTGAATGCGAAGGGTTTGAGCTGAAGGGCGCTTCCAGGATCCAGAAGGTCACTGGAGACCTGTTTTTC
ACCCCCTCAGAGGGCAAAACCAAAAGAAAAATGGATTAGGAGAGGGGG
HUMAN SEQUENCE—mRNA
ACATTTAGGAGAAACACCGGTGTCTGCGGCTCCCACCCTTCGGGGGGCCCGTGGGGGGGGCGGTCTCACGGCCATGGACGCCACCCCCCAGGGGTCTC
TGCTGCCGGCTACTCTCCTCTCCACGTGCTCCCCTCCAGGACCCCCGACGCCGCCTAGGCGCCCAGCGACGCGCGGCAGGTGGCAGCAGCTCGGGCCC
CCGCCCCACTCCAGGCGCCCGCAGCGCTCGCCCTGACGCGGCCGCCATGGCGCAGGAGAACGCGGCCTTCTCCCCCGGGCACCAGGAGCCGCCGCGGC
GCCGCGGCCGCCAGCGCTACGTGGAGAAGGATGGCCGGTGCAACGTGCAGCAGGCCAACGTGCGCGAGACATACCGCTACCTGACCGACCTGTTCACC
ACGCTGGTGGACCTGCAGTGGCGCCTCAGCCTGTTGTTCTTCGTCCTGGCCTACGCGCTCACCTGGCTCTTCTTCGGCGCCATCTGGTGGCTGATCGC
CTACGGCCGCCGCGACCTGGAGCACCTGGAGGACACCGCGTGGACGCCCTGCGTCAACAACCTCAACGGCTTCGTGGCCGCCTTCCTCTTCTCCATCG
AGACCGAGACCACCATCGGCTACGCGCACCGCGTCATCACCGACCAGTGCCCCGAGGGCATCGTGCTGCTGCTGCTGCAGGCCATCCTCGGCTCCATG
GTGAACGCCTTCATGGTGGGCTGCATGTTCGTCAAGATCTCGCAGCCCAACAAGCGCGCAGCCACGCTCGTCTTCTCCTCGCACGCCCTCGTCTCGCT
GCGCGACGGGCGCCTCTGCCTCATGTTCCCCGTGGGCGACTTGCGCTCCTCACACATACTCGACGCCTCCATCCCCCCCAAGCTCATCCGCTCGCGCC
AGACGCTGGAGGCCGAGTTCATCCCGCTGCACCAGACCGACCTCAGCGTGGGCTTCCACACGGGAGACGACCGCCTCTTCCTCGTCTCGCCGCTGGTT
ATCAGCCACGAGATCGACGCCGCCAGCCCCTTCTGGGAGGCGTCGCGCCGTGCCCTCGAGAGGGACGACTTCGAGATCGTCGTTATCCTCGAGGGCAT
GGTGGAAGCCACGGGAATCACATGCCAAGCTCGGAGCTCCTACCTGGTAGACGAGGTGCTGTGGGGCCACCGCTTCACGTCAGTGCTGACTCTGGAGG
ACGGCTTCTACGAAGTGGACTATGCCAGCTTTCACGAGACTTTTGAGGTGCCCACACCTTCGTGCAGTGCTCGAGAGCTGGCAGAGGCTGCCGCCCGC
CTTGATGCCCATCTCTACTGGTCCATCCCCAGCCGGCTGGATGAGAAGGTGGAGGAGGAGGGGGCCGGGGAGGGGGCGGGTGGGGAAGCTGGGGCTGA
CAAGGAGCAGAATGGCTGCCTGCCACCCCCAGAGAGTGAGTCCAAGGTGTGACCAGCTTCCTCCAGACCCCTGTGGCAGACCGGGGGCCAGACACAGA
TACATGGGGAACTGCATATCGGAGGTGGTGGAGGAGGAGGAGGAGGAGGAAGGCAAAGCCCCTGGAAATGTGCTAAAGTTGGAAAGTCCCCGTCCCCC
AGAACCTCAAGTCTAGAAACCAGTATGGAAGGGAGGGGTCCTGATTTCAGGGAAATGGAGGGTGGGGCCGGGTGAAAATGCCAGTCTGTGTTTGACCT
TCACATTTGTTCATGAGTGGATGGATGGACAGAATGATGGACTTTTGGGCGTTGGATGGGAAGATGGTAGCAGATAAAGACAGCTGACAGATACATAG
ATGGACCAGTAGACAACTGGTCCACTCAGGGCTGCCACTAACCTGTAGAACACCCCTGTGCAAATTTTAAAAAGGAACCCTTTTCCTCCAGACAGATA
CAGCCCCAAACCAGGGTGCATGGCTTGCGGACCAGAGTATAGGATGGATTGCAGTCCCCAGTCACCTCTTCTGCCAGCCTCCCCACATATGGCACAAC
TGTCTAATGACACGGTAGGCCAAGCTGAAGTGAAGGAGAAAGGAGCCGGACCAAGATGGGCACATGAGGAGGGTGCCCTCCTAGCTCCACCCTCACCA
GGATGAAGGCGTGCAAGGCGCTCAGCAAGGTGTGAATGACCTTAGTCCGCAAGTTCAGGGAAGCAGGCAGAGCGGGGAGGTGCCTGAGCTGGGGCCTG
GAGAGGGGCCTGGGAAAGGAAAACCAGGGATAGCTATTTTCTTACAGTGGAGTGAGATCTTACAGGTATCAGGCACAGGCAGGAAGAGAGAGAGAGAG
GTTCTGGGGAGGAAGGGCCAGGAGAGACATCTAGAAAGTGGGTTCACTAGAGCTGGGAAACAGGGAGCCCCTAGGAAAGCAGTGTGTCCTTGGGGCAC
AGTCATTCACATCACTGATTGGGTGCCATGTGGAGTGGACATTCAAAAACCTGGTTCCTGTCCTCAAAATAAGGGGCACCTGGGAAAACAGAGGAATC
TACCTGTGGTGACTGAACGAGGGATAATTCAAACTGACAACCTGTGCAGTCCCGTGGAGGGTAGGGGAGTGTCGGTGATCAGAAGGCTGGGGCCAGTG
TAAGGCATAGGGAATATGTAAGTCAGGAGTTAGAAATCTCCAGTGTGCGTTGGAATCACCTGGAGGGCTTGGTAAAACACAGATTTTTGGGCTCCACT
CCAAGGGTTTCTGACCCAAGAGGTGGGGACCAAAACCATGCATTCCTAAGAAGTCCCCAGGTCATGCTGCTGTTGCTGGACTGAGGACCACACTTTGA
GAACCTGTGCTCTAAGTGAATACTTGGAAGTCGTTTCAGGACATGGCGCATAGAAACTGAGGAGTAGCTGAGAGGAAGATGAAGAGAAGCTGAGAAGA
AGCTGAGGATCCTCACAGGAGCAGACAGAGAAATGTGAAGGGTGGGGTTTTATGTGTGGGAAAGGGACCCGAAGCCCAGGCTGAAGAGTTTAACTTTG
GGCCCAGAAACTCAACCATCAATGGAAACAGGGCAGTGACAAGTGGAGGGGGTGTCTGGAAGCTGAGCAGGCCCGACAGAGAGATGAAG
HUMAN SEQUENCE—CODING
ATGGCGCAGGAGAACGCGGCCTTCTCGCCCGGGCAGGAGGAGCCGCCGCGGCGCCGCGGCCGCCAGCGCTACGTGGAGAAGGATGGCCGGTGCAACGT
GCAGCAGGGCAACGTGCGCGAGACATACCGCTACCTGACGGACCTGTTCACCACGCTGGTGGACCTGCAGTGGCGCCTCAGCCTGTTGTTCTTCGTCC
TGGCCTACGCGCTCACCTGGCTCTTCTTCGGCGCCATCTGGTGGCTGATCGCCTACGGCCGCGGCGACCTGGAGCACCTGGAGGACACCGCGTGGACG
CCGTGCCTCAACAACCTCAACGGCTTCGTGGCCGCCTTCCTCTTCTCCATCGAGACCGAGACCACCATCGGCTACGGGCACCGCGTCATCACCGACCA
GTGCCCCGAGGGCATCGTGCTGCTGCTGCTGCAGGCCATCCTGGGCTCCATGGTGAACGCCTTCATGGTGGGCTGCATGTTCGTCAAGATCTCGCAGC
CCAACAAGCGCGCAGCCACGCTCGTCTTCTCCTCGCACGCCGTGGTGTCGCTGCGCGACGGGCGCCTCTGCCTCATGTTCCGCGTGGGCGACTTGCGC
TCCTCACACATAGTGGAGGCCTCCATCCGCGCCAAGCTCATCCGCTCGCGCCAGACGCTGGAGGGCGAGTTCATCCCGCTGCACCAGACCGACCTCAG
CGTGGGCTTCGACACGGGAGACGACCGCCTCTTCCTCGTCTCGCCGCTGGTTATCAGCCACGAGATCGACGCCGCCAGCCCCTTCTGGGAGGCGTCGC
GCCGTGCCCTCGAGAGGGACGACTTCGAGATCGTCGTTATCCTCGAGGGCATGGTGGAAGCCACGGGAATGACATGCCAAGCTCGGAGCTCCTACCTG
GTAGACGAGGTGCTGTGGGGCCACCGCTTCACGTCAGTGCTGACTCTGGAGGACGGCTTCTACGAAGTGGACTATGCCAGCTTTCACGACACTTTTGA
GGTGCCCACACCTTCGTGCAGTGCTCGAGAGCTGGCAGAGGCTGCCGCCCCCCTTGATGCCCATCTCTACTGGTCCATCCCCAGCCGGCTCGATGAGA
AGGTGGAGGAGGAGGGGGCGGGGGAGGGGGCGGGTGGGGAAGCTGGGGCTGACAAGGAGCAGAATGGCTGCCTGCCACCCCCAGAGAGTGAGTCCAAG
GTGTGA

TABLE 6

MOUSE NOMENCLATURE
ICSGNM Ppp3cc
Celera mCG3861
HUMAN NOMENCLATURE
HGNC PPP3CC
Celera hCG15020
MOUSE SEQUENCE - GENOMIC
TTAGGTAGATAGTTGGCTTTATACTTGCTACATATGCCAGCTTGGCTTCAAACTTGAGCTGATCCTCCTGCCCCTGTCTCCTGCCTCTGCTTCCCAAG
TGCTGAGATTACAAGCTGTCTGTACGGTTGTACGTGGCCTTTTTATCTTGTTTATGTGAATTATTTTATCTTACTTAACTAGAATGTAAAGAGGCTCC
TGTGTTAATCTTATATATGCCGCATGCTACCACACAGTGAGATAGATAGTAGTAGATAAATATTTACTTAACTATAATGTAATTATTTCTTTGGTTAG
TATTTTTCCCTTGGCATTTTAGAATTTTTACTTTAAAAACCTTTCTAGCTCATTGTCTACCAAAGATTCCAGAGCTTCACTTTAGACAAACATCACTG
TTACTCAGAAGTCTAGTCTGGGTATAGAAACGGATTCATTAGTATGTAGTGTTCATGTTAGTAAATCTCCCAGCCTTTTATCAAGATGGTACTGATGA
CTGCACTTGAACTGTTAATCTCCTGCCTGCCGTAGGAGATAGAGACTGCGATGTCTAAGCTTCATAAAGAAACGGAGTGTGTGACCTTGCACACCAGT
CGTCCTTAGCTCTTCTTAGTAAGGAAGCAGGTGATGGTAGTTGTGGCTAGTGCATTTCCTCGGTGCGTGTTGCATTTTTGTTGGTTTGTTTTGAGACA
GAGTCCCACGTAGCCCAGGGTAACCTCTAGCTATGTAGCTGAGGTGGTTGAGCCAATCCTTCCAGTCTTAACCATGCCTGGTTAAGTTTGTTGCTTTT
AAATACAATCAAGGAGGTCGGCAGTCTGAGTCTGGAAGTGAGTGACAGAAGAATAAAATGCCCCGTCGTCGCTTCAGGGAAGAGGGCAGTGGGCTTGG
GGCCTGTTGCACATAACTGACGTTGACACCATAAAGGTGTCATGTGGTTTAGTATTTTTATCTTGGCTCTTGGTTCTCTTTCAGACTAGAGCTTCAAG
TTTCTTACAATATTTTGGTGCAACTCAGGGGTGAAGTGTGTTAAAAATAACTTGATAATAGTTGATATCCTGGACTTTTGTTTCAGTCTCTGAAGAAT
TGGATGCAAGTCAGGTTTATGATGTCTAACCCTTTCCTCTTAATGCCTTGTGCATGCTTCTTTTAAGAATAGAATTCTTGGAGGTTTATCTAGAACTG
TGTCAAATCCCGTTTCTTTTGATAACTGCATCTTGCATCTGTTCTGTCTACATAGTTACTGCTTACCTCAAACTTGCTGATTGATCTCTCTGATTATC
TTCATTCCAGCCCACAGCAGAATGCTCTGATTTGTGTATTAGGAAGAAGGGATCTGGGAGATGGCTCAGTGGGCAAAGTGCTTGCTATGCAAGCATGA
AGGCCAGAGTTTGAACCTAGCACTCCTGTGGTAGCCAGTGCCTGTACTTCCAGTGCTGAGGAGGTGGAGGCTGGTGGGTCCCGGGCACACATGCACAC
ACCCAAGGAAGCCAGAAGAGGGCGCTGGGTCCTTTGGAGCACTTGTGAACCTCCCTATTTGAGTACTGGGAACCAAACTCTGGTCTTCTGAAAAGTGT
TCATAACTACTGAATCATCTCTCTATCCCTATCTGTTCATTTTTTTAAAGACACATCGCGCGCGTGTGTGTGTATGTGTGTGTGTGTGTGTGTAAAAA
TTAGGCCAGACACAACAGTGGGTAGCAGATAGCTTAAACATCAATAAATTCATCTTTAAAATGTGGTTGTGGAGGCCATACAATAAAGTTAATGCAGA
TTTCAAATTTTACTTTGGTTAAAATGAGATTATGGAATTGATAGGCTACTGAGAAATTTAACTGAAAATCTGAAAGAAAAGGACAAATAGCCCCTTGG
TCAGTGAGATTAATAGTTTGTTTTAGAAAGATAAGATGTACTTTTAAATATTGTCTAGGTTGAAATGCCTTCTTTTAAAAGGTATGCTTAGGGAGAGC
ACTTGTAATGTGTATGATGGGGTTCCAGATAGCTTTAATATGTGAGTTTATTTTTAGTCATCTATATTTAAAGAACTGTAAAATTAGCCATATGCATG
AAAAGTAGGCAAAAGATACCAAAGTCCTCACTGATGATTCATGGATGAGCAGAGTAGACTGGCCGGTAAATACAGCTTACAGCTTGCAGGGACATTAT
TTCTTTATCAGCAAAGCAATCAGCAGGGAGCCTTTTAATATTTCACAGCAGCAGACTACCTAGGTGATCCTGGCTCTGTAAATTATATTGTTATCAGT
ATTTATTTGTATAAGAAAATTTGAGTAGCTTGCTACTAAATTAAAATATTTACAGTATAACTGACTGTAAAAACTGTATTTCAGCAACACAGGTAATT
ATGCTTATTTTATTATGTTACCAGTGGTCATTTTAATTGGTAGGCTTCTAAGAGATTTGTGTATTTTTTTATACTCCTTAATTTTAAAAAATAGTTTT
CTATTCTCTATACTGTGAGTGTGTGTGTGGTGGTGGGATCAAATGCAGAGCCTTGAATATGCTAAGCAAGAGTTCTACCACTGAGCTGTATCCCTAGC
CCAATATATAATATCTGTGATTCTAAGAATTCTTTAGGGGCTAAAGAGATGGCTCAATAGTTAAAATCTCTTACTGTTCATGCATGTATACACACACA
GTGGTGTTCATGCATGTATACACACACAGTGGTGGTGGTGGTGGTGTTATTTTGAGGCAGCATCTCTTTATATAACCTTGGCTATCTTGCCTCTGCCT
CTAGTGCAGGGATTAAAGGTGTGTGCTACCACATAGAGCTTTAGTTTTACTTTTGAAACAGAATCTTTCAATAGCCTTGAGCTCACCAAGCAGGCTAT
GCAAACAGGATAGTATGCTCTGGAACCCTATCTCTGTCTCTAAGGCACTAGGATTATGTGTGCCTGCCAACCTTTTTTATATGGGTTCTAAGAATTGA
ACTTGAGACCTTGTGCTTGAAAGGCACTGAACCATCTCACTAGCCCTGTGGTTTGCTTCAGTAAGTTAAAGACCTCAAGCTCACTACCACCTTGAACT
GCTTTATAATTTTCTAGTCTTACAGAAATTGTGAATTTTCCTATCTTCTGACATCTGTCAATATTCTTATTCATAGGTAAATTCCAACTGAATTGAAT
ATTAATAGTATTACTGTTCATTGACAAGCTTACAATAGAGTGAAACACAAGTCTAAAGTAATGTAATATTGACTTAATAATGACTATATTATATAATT
TTAAAAGGTATTTAGCAAAATTCATATCCCTTTCATCAATTGCTTCCTTAAAATATAAAGCCCACCACTGAAGAACACCAGGAACATGGTATAGTGAA
AAATAAACTTTATTTATTTAGCTTATTGTAGAAAGTATTTTCACCCCCTAAAAGCTGTGAGCATGTTGAGAGGAGTCAGAAAGTCTGCAGAAGGTATA
AAGGAGCTGAAGTCAGGTTAGTAGTCTGCTCAAGGGTGGCTCAATTCTCATTTGAATGGTTTTGCTGTTATTCTGCACAGGCTCCCTTGAAGTTGTAG
ATCAGTTTTTGGACTGGGATACTGAGTGTCTGTGGCCCTCAAAAAACAGTGCTTGAGCTTGGGTGACCTGGACTGTACAGTGGTGTTTGCTTTGGTTC
AGTTTCTCTGCTTGCAAGTCAGTGGTTTGTGGCTTCTGTTTTCTTTCTCTGTCTCCACCTCTGATCATTCTTTAGTTAAACCAGGGTATTTACCACTT
AAGGGAGGTAAACCAACGAGATTTTAATAGGTTGTAAATGCTTATCAAGCCATTTGTGATAGATATGTTGACCTCAAGCACTGGCCTATTTCCTGGAT
GGTGATTTGTTCTGCCATCTGGTTGACAGAAGCAACACAGTGGCATTGAGGGATCTGGAGATGACACACTGGGTTATCCCAGACAGAATTTGTATTTA
GTTTGAAATAATGTTCCATATATGTCATAATCAGTTTGGATCTTCCTTTTTATTTGTTCATCTGCTTGTTTTGTTTAGGGAAAAATATTACGCGTGTG
TGCTCATGATGTGTGGGGATGTTTGCATGGTGTGTGTGTGTATATGCATGGGCACACTCGGGTGCATGACGTGTGGGGCATGCATGTGTATGTACATG
TGTGCATGCTTTATGTATGCATGCATGGGTGTGGGACATTCATGTGGAAGTCAGAGGACAACTGTGTGAAGGCGGGGCTCTTCCACCTTCAAGTATAT
TCCTGAGATAGAGCTCTGGTCACCAAGCAGGAACATGAAGCTCCTTTACCTGCTGAGACATCTTACCAGTCCACAATTGTTTGCAATTTGATTTTCAT
TGTTTACCTTTACAAGTGTCTGAATGATAATTCTAGTATTTAGTGATGGTCAAGGGTAAAGGTCTTTGTAGTACTAACAATAAAGGAGGGGGTGGGAG
CCTCTTGACATTCTAGAATCTTGACCCATAGGGAAGAGAACAAGATAGACAAGAGATTTACTTTTTTTGCTTGCTCAGTTATTTTTCTCTCAAACTCT
TAAAAAAGAAAAAAAAGGTTAGACTTATTTCACATGTTTCAACAGTCAGAGAGATTCAGAGGAAAGTGGGTATTGTCTGAGAAGGAAGCAGTTATAGG
CAGGCACTCATCATGTACTGCTGCTAGGATAATAACAGTTGTCAGATTTTAGGTAGACATTTGCCATCTGGGCCAGCACCAGGAGAGTGTGTAAACAC
AGTTGTTAGACATTGCAACACTCTAGTGTTAAGTTGACTCCAATGCTTACAGTTGCCGTTGACTTAATGTGCTAGAGGGTCTTGTCTTAGACATGAAG
CTGAAGGCTTTGCTTTGGGTTTTAATCATCTGTGATGTGTGTTGTGGGTTGCTGTGCTCACTTTGTTTTATGAATCATCGTGCAGACAAGTTGTGGTT
TCTCTTTTAATTTTTTGAAAAAAAAATTGATGATTTACATTGTATGTGCTTTGGTGTTTTGCCTGCATATGTATGGGGTGTCAGGTCCCTGGAGCTGA
GGTTACAGATAGTTTCGAGGTGCTATGTGAGTTCTGGGAATTGAACCCATTTCCTCTGAAGAGCAGCCAGTGGTCTTAACCTTTGAGCCACCTCTCCA
GCCCTTAAAGATGTATTTTATTTTATGAGTATAAGTATTTTGCCTGAATGGATGCACAATACTCAGTGCACCACATGCATGCCTAGTGCAGTGGGTCC
CCTGGAGCTAGAGCTACAGACAGTTGTGAGGAGCCATGTGGGTGCTGGGAATTGAACCCAGGTCCCCTAGGGAAGCAGCCAACACTCTTACCCATTGA
GTTATCTCTCCAGCCTCTCTCTTTTAATTTTAAAAGACAGCCAGTGTGAGTTTCTTGTGTATTTGTGGGGTAAGGGGTTCATCTCATGGTAGAGCTAT
ACTTCATGCTCAGTGTGACAGAAGAGTGTCTCTGTAGGCTAGCATGCCTGTAACCTTGGATTGTGGAGGCAGAGAAGGAAACCAATGCAATGTTAAAA
CTAGCTTGGTCTGTGTTCTGAGTTTAGGCCATCCAGGGCTATAATATCAGGAGACTCTACTCAAAAAACAGTCAGAAAAACAAACCACCACCAACCGA
TCAAACAAAACCAAACCAAAGAATAAGTATTTGTAATTATTGAAAAGCTGGAAAAAGAACATTTTGACTTGAAGGATTAGTCAAGGGCCTGTATTAGT
TTTCTCTCCTTTTTCAGATAGGGGTAGACATTACTGGGTATTAAATTTAGGGCTTCACTTATACCCAGCAAGCACTCTACCTCTGACGGCCTTTTCAC
TTCCAGTAAATGGCCATAGTGCCAGCGAGGCTCCAGCTCTCACAGGTGTACACCACTCTTGGCTACGGGGGAAGCCCCTTTACTTCCCCTTGAACCTG
GGAAGCTTGAGATGCTCGAATGGTCTGAGTTGGAAGTGGTGTGGTGTGGCTAAGATGCTTGTCCTTGGAACTCAGTCACATGAAAGGCCACCTACAGG
TATTCTCATCAGATGAGACTGCCCTGGGCTGTATGAGCTGGGGGGGGGGGGGGGAGGCGGGAGGATGGTGGGGGAGCAGTCCTGCTGTGTCTTCTCTG
AGCTCATCGTACTGAGTCTATACATGAGAGAACCTATACATAATTATTGTTTTAAGTCACTATGTTCAGGGTGACAAATCAAAGATTGTGGGAAAGAA
ATATACAAGGAGTTTAGCAGAGGGTGGTCATGCTAGCCTCAGCTCTTGGGAAGCTAAAACAGGATTGTCTAGCGTTATAGAGATGCTGTCTCAAAAGC
AACAGCAACCCAAGGATGTTGGTAGCAATATTAACTTATAACAATAAGTAAAAATTTTAAATAGTCTACTTAATAGTAAGAAAATAAGGGGCTGGAGA
GATGGCTCAACGGTTAAGAGCACTGACTGTTCTTCCAGAGGTCCCTGAGTTCAATTCCCAGGAGCCACATGGTGACTCGCAACTATCTGTCATGGGAT
CCGATGCACTTTTTTGGTGTGTGTCTGAAGATAGCTACAGTGTACTCATATAAATAATAAATAAATAAATCCTTAAAAAATAAAGAAAATAAATAAAC
TGATACATTTTTTTCCATAAAGAAAAGATCTGTCTTCAAAAGAAAACAATCAGTTCTGTTATAACCAATGTCAAAATGGTCCCAGTGTACACACATGT
AAATAAAGATGATTAAGAACAACAACAACAACAAATTGTAGGCCTGGGGAATATAGCTCATCAACAGAGGCTTCCTAGAGGGAACAAGGCCTTGGATA
GAGCCCCAGCATTACCAAAATCCAAAACAGTCAGCAAGTTTAAAAAAAAAAAAAGGAAGGTTCCAGGTGAGCTGATCCATAACTGTAACTGTGTAAAT
ATCAGTACCACAGCGCTTCAGCTGGTCTGTGAGGGAGGACACCCTCATTATGTAACGTTTAAAATCATGGCTTTAGATGGCTTAAAACCTTCCTCTTT
AGACAAGTTTGATTTTGAACAACTAGAAGAAGTGTACAGATTGAGCACACAGTATTAAAGCTGCCAGCCCCTCCCTACTGCCTTCAGGGCAGTCTCTG
GGCTTCTTATTTTCTCTGTCTCTGTCTCAGGATTTTCTTTTTTAAACTTTAAATGTTGAAACTTAATCCCCAGCCCTCATGCATGCTCTGCCAGCCCT
CTTGGATCATGCTATACACTTCATGAATCTTAACTGCTTTTATGATCTTATAGGGACTTAAAAGATTGGTTTTCCTAAGAAGCACCAACTTGTCACTG
TAGGCGCACAGTAACTAAAAGCTGAATAAAACATTGGCATCTGTGACTTTATTTTGTCTGTCTCTTCTGTTATGTTAAACTTCAGGTCTGATTCCTGC
TCAGCTGTGCACATAGACATATTAATTACTTAAGGAGTGTCCATCTAAAAACTGCTTATATAAAATTTGTTAGCCTTAAATTTGCCTAACCTCTCTGT
AAAATACAAGAACGAAGACACGGGAATCTAAGGTACTTTTTTTAAGTTCCAGAATTTTGTAGGCAGATCACACAGTGATGTTACTACTCTGCCAAGGA
GGAGCAATTATCCCATGAAGGAAACTAAGGCAGATTCCTTATGCAAAAGTAATCTCTTGATGAGAATTCTATCACCCTCCTCAAGCAGTAAAGACTCT
TATGTAGCCAGCAGGACCTTGCAGGATCAACCAAAATAATGGTCAACTCGACCATTCTAGTCAAGGTTAGCTGTTTATCAGTCAGGCAAACACACCAG
CTGTGCAACACCATCCCTTCTCCTCATTTCTCTAAGACTCAGAGTTCCTTCCAGAAACTCAAAAGCAGATCTTAGGTTACCAGACATTTCTGTACTTC
CTAGTTCAGCCTTGATCTGTCTCTTCAATTGACTTTGGAGACAGGTGGTCAAACCTAGTCTATTGTGAACCTTCAGGTCAGGGATTGTGTCCTAAACT
CCTACAAAAACCTTGCACTTCAGCTTTCTATTTAAATATAGATCTTTTACTACATCATCATCATCATTTTCTTATGTTAAAACACATCTCTCACTTCA
AAGGAAATAAGAGCCAAATATGAGTGACCATGGTCTTTGCTGTGACCTGACATTTGACAAATAGCAACTGAAGAAGGAATGGTTTGTTTTGGCTCCTA
GGTTGAGGCTGTAGTATATCATGGTAGGAAAGCAAGAGTGGCTCCAGCTGCTGTGGCCCTGGGTGCCAGCCCACACGATCCCCTCACTCTTGAACTTT
GCTTAGCTGTAAAACCATCACCACGTGGACAGTTGTTCTGGCAACTCAAGATAAAGCTTGGGTCTCTCCTGCAGATATCGTAGCCTCTGTGCCCGTTG
TATCTGAACTGGGAAACACTTTCTCTAGGTGGTTGATTTTTGAGTGTGGAACCCCTTCAGCTGCATTCTTTCTTCAACCCCTTCTTTCAAAAGATTTT
CTACTTTAATAAACTGGGGCTATTGAGTCCATTTCCTGTTGGCCCAGTACAAATGGAAGGTTGTTCTTTAGTGATGCTAATCTCATACAGTAGAGAAA
TAGTCAGGGTAATAGCCCCACTTCTCAGTACCAATTTTCATATAGTCAGGGTTTTATAGAAGAAGCAAACCAATAAATGTGCATATTTCTGTTATAAA
AGGAGATGTATTAGGTTGACACATGTAACAGGGCCCAGTGGTGGCCATCTGTATGGTGGAGAGCCGAGAACCCACTAGTTGCTCTGTCCATGAAGCTA
GATGCTTTACACAGTACCAATCTGGCACTGAGGCCTGGAGCACCCGAGAAGAGGCAGTGCTAGGAAGCTGAAGTAGCAAAAGATGCCCCTGCTGAGAA
GTCACGAGGGAGGCCGCAGGTGCACACTACCTTTCCCTTGGACCCCTTTATTCTGGATTTCCTGTTGGAGCGTGCCCACTGACTTTGAGAATAGGTTT
TATCCCCTCAGTAAAGCCCCTCTGGAAGCACCTTCACAGACATACTGAAAGGTCTGTCTTTCTTGGGTGTTTTTAAAGTCTTCAGATTGCCGAGATGA
ATAACTCTACACTCCAATAACATTACAGCATGAAAGAGGTTATGTGGACTTTTTATAGTTCCAGGAGGATCAAGATATGGAGCCAGCCAGTGGTGGCG
CACGCCTTTAATCCTAGCACTTGGGAGGCAGAGGCAGGTGGATTTCTGAGTTCGAGGCCAGCCTGGTCTACAGAGTGAGTTCCAGGACAGCCAGGGCT
ATCCAGAAAAACCCTGTCTCGAAAAAAAAAATAATGAGTTAGCAGGTTACAACAAAGTTAATAAGTCTTAGATGCTATATGAATGAGATTCTTAGCTT
TGGGGTTGGTGGAATCTAGCAGCCTGCTAAATTAATTCATTTCAAAGGTTTTGCCTCATAGTTGAAAGTTATGAGAGGTCAAGCATTGAAGTGGATAT
TAATGGCTTCAGGGTCCTATGTTGTCCAGTTTGACCTTCCACTATCTTTGTGTCTGAGGATGACCTTGAGTGTCTGTTCCAGCTGCCTCTACATTCCA
GGTGATGATGGGATTAGAAGTGGGAGCCACCCAGCCTGCTTTATGTGATACTGGGGATCGAACCCAGGGCTTCTTCTATTCAAGGGAAGTACTCTACC
AACTGAGCTCCTTCTTCAGCCAGCTGTTTCACTTAGACAGATGTATTTGTTGCGTTCATTTCTTCATCTATTCATTCAGTGGATGTGTGTGGATGTAG
TCATGAATGTACAGACAACCACATAACTTGAACTGTGACCATTTTAAAATTAGAATGATCAATTCTGCTACACAGCTTCTTAGACTTTTTCACATTCT
AAGACTGTGCAGTTATGATTGCTTATTAGGCTTAGTTTATTTTAAGAAGTATGACTGTGCTTGTTATCGGTACATTTAAATATTTGAAATTGTTTTCT
CTTCCTTAGCTGTCCCCTTTCCTCCAACCCGACGGCTAACTTTGAAGGAAGTTTTTGAGAATGGGAAACCTAAAATGGATCTTTTGAAAAACCATTTA
GTAAAAGAAGGTCGGGTGGAAGAGGAGGTGGCCTTAAAGATCATCAATGATGGGGCTGCCATCCTGAAGCAGGAGAAGACCATGATAGAGGTGGAGGC
TCCGATCACAGGTATGAAACACATGTGAATGAAACGCACACGTGTGGGTGCCAGCCCTTAGGTTTGTGATAGTTTGAGAAATGATTACCAAAGGCCAG
CTGAAATGAGTGTGATAAATATTTCTCTAATTTTGAAGCAATCTAGGGATTTTGACTTTTGTTGCTTAAAAAACAACATTAGGGTCTGAAACTTGGTG
GATAAGAGCACATCCTGTCTTGCAGAAGACTGGAGTTTGGTTCCAAGTGTCCATATTAGATGGTTCAGAGCCACTGTAACTCCCAGCTGTAGGGAGAA
TACCACACCTCTGGCCTCTATGGACACTTGTACTCATGTACACACACCCCTCTCCCAGACAGATGCACACATACACACAAAATTAAAAATAAATCTTT
TTAAAAAGAAGCATACTCTTAACTGCTTCATATTGATAATAGGGGCATTGAGTTCTGATTGAGTAGCTAAATATACAGAAGAGCAGTGAAAAGGAAAG
TGATTAAACTGAAGTCCTGTTGCCCTGATGCTTCTTCCTAACGCACAGTTTCCACCTCATGTCTTTCTCGCCTAGTGTGTGGTGATGTTCATGGACAA
TTCTTTGACCTGATGAAGTTGTTTGAAGTTGGGGGATCACCTAGTAATACTCGCTACCTCTTCCTGGGTGACTATGTGGACAGAGGCTATTTCAGTAT
AGAGGTAAAATTAACATGGCTGTACTGCCCCTATTGTATCATATTCTAAAAGATCTGCTCTCCATTCCTTAGTAGAAAAGATTAAGATATGAATGCTG
ATAGAATTTATCCACTTTATTACATTCAACTAATAGAGATATGCTTTTTTTCTCAAAAAAATCTATCCTTTTAACCTGTAGAAAACCTGTCATATGGG
AGGCTATATTAAGCTAATTTTGAAAATAGATATTTGCTAAGGTTTTCTCCTTAAAGATAGCTGTCTGGGATATAGGGACATGCATAGCTTTTCATTCT
AAACAGTGTCTGAGCTAAGTACCTTAATTATAATACTGCAGCCAACGCTTAGGTTAACTTCCTGTGGGAACTAGGCACTGCTGGCTGAGCTACTGCAG
GAGGGACTCTGGTCCTGTTGCACACGGCTGGCATTAATGTCCACTAGAAGGCAAAAGATGAAGTGAAGCCAATAAAAACTGACAGTTTACCTTTATGT
TTTAAGAATTAAATGATGGAACATGAGTAGGATTAAAAACAGGTATGTGCTTTTGTGGGTGAACATAAAATGATTTTAGGGCCCAATACAAAGGCTTG
AGGACCATGTAATTATAGCACATCTGCTTCACAAATAAAAGATCCTGTGTGAAGAAGTCTTCATAATTAAGTTTGTAATATGCCCTAGAAAGAACAGG
AGGTGACAAATTCTGTCGTGTTTTAGTGCTTTAGGCTATGTGCTCTTCATAGTGTGGGGATTTCACTGTATTGGCTGCTCCTGCTAGAGAAAGGTCTC
CTCCTCTTCCTCCTCTTCTTCCTCCTCTTCCTCCTCCTCCTCTTCTTCCTCCTCTTCCTCCTCCTCTTCTTCTTCCTCCTCTTCCTCCTCCTCTTCTT
CTTCTTCCTCCTCTTCCTCCTCCTCTTCTTCTTCCTCCTCGTAGTCCTCCTCCTCTTCCCCTTCCTCCTTGTAGTCTTCCTCCTCTTCTTCCTTCTCC
TCTTCCTTCTTCTTCCTCCTTCTCTTCCTCTTCTTCCTCCTCTTCCTCCTCCTCTTCTTCCTCCTCTTCTTCTTCTCCTCCTCCTCTTCCCCTTCCTC
CTTGTTGTCTTCCTCCTCTTCTTCTTCCTTCTCCTCTTCCTCTTCTTCCTCCTTCTCTTCCTCTTCTTCCTCCTCTTCCTCCTCCTCTTCCTCCTCCT
CTTCTTCTTCCTCCTCCTCTTCCTCCTCTTCCCCTTCCTCCTTGTTGTCTTCCTCCTCTTCTTCTTCCTTCTCCTCTTCCTCTTCTTCCTCCTTCTCT
TCCTCTTCTTCCTCTTCTTCCTCCTCCTCCTCTTCCTTGTCCTCCTTTTCCTTCTATTGTTTTTAAATGTAACTCAACTGTCTGACTCAGTTTTCAAA
GAAGAGTCAGTTCTGAAGACCCCAGAAGAATGAACAACAACGTATGTTTATTTTACTCTTCAGTTAAGTAGCAGTATAGGGAGCTAGGGTGTAAGTCA
CTGCCAGAGTTCTTGTCTAGCATGCCAAGCCCTGATGTTAATTCCTGGCTCTGAAAAAACATTGTGTGTGTGTGTGTGTGTGTGTGTGTGTGTGTGTG
TGTGTAGGGTAGGCATTCTAAATCAGAAAAGTTGAGTTTTATCTGTTACTACCCCAGACTCATTAGTGTCAGATTGAGAAGGACACAGGATACTGCTA
TAGACTATGACATTTTCCTGCTGAGCCTTGTCCTGCTGCTCATGCTCACAGTGAGCACCCGGGGATAGCCTCTGGGCAGAGACTGGGATGGGTGGAAG
GCATGTCTTGAGATGTGTCACTGAAGACCTTGCCTATTGCAGTCAGGAATTGACTGCAGGGCTGTGCACTTCAGTAGATAGTGCTAATGGGTGGGTGT
TTAAAAGGTAGAAGATAAAGAATAAAGAGTGTTTTCAATTATCTATTCATTGTTTTACTTAATTAAATATATTTTTGTTGTTTTGGGCCTCCTCCTCC
TCCTTCCTCCTTCCTCTTCTTCTTCCTCCTTCTCCTTCCTTCTCCTCCTTCCTTCTCCTCCTCCTTCTTCCTCCTCTTCCTCCTTCTTTGTATTATTA
TTATTATTTCTTTTGTTTTTAAAGTTGTATTTATTTATATGAATACACTGTAGCTGTCTTCAGACACACCAGAAGAAGGTATCTCATTACAGATGATT
GTGAGCCACCATATGGTGGCTGGGAATTGAACTCAGAACCTCTGGAAGAGCAGTCAGTACTCTAACTGCTGAGCCATCTCTCCAGCCCTATTATTATC
CCTTTACATCCTGACCACAGTTTCCCCTCCTTCCTCTCCTTCCAATCCCTTCGTTTTTGTTTTTGTCTAAGTATTTCATTTGGTTTAGGTTAGCCTCA
GAGTTGCTGTATATCTGAGGATGACCTTAGTAAACTTCCGATCCTTTTGTGTCCATGCTTCCATGATCCTTACATAGTGCTGAAAATTGAACCCAGGA
TTTTGTGCATGCTAGGGAGATACTCCACCAACCGAGCTAATTCCTAGCCCCAGGGACAATGTATTAAACATAAACATTAGTGCTGGAGAGATAGCTCA
GCAGTTAAGAACACAGCCTGCTTTTGCATAGGACCTGGGTTCAACCCCTAGCACCCATATCTTCAAATGGCTCACAACCATCTTTAACTCTTGCTCCA
GATGATTCTGGCCTCTGTGGGCAAATGGTCTCATGTGCACACAAGAACAAATGTACAGAATCTTAAAAAATACAATCTTGAAACAAATAGGACAAAAT
ATTTTCTAATTATTATGACTTACAGTTAATTAAAAATAAGACTTTATAAAGTTTTAATTTTAATTTTGTTTACTTTAAGTGCTAGGGATTGTCCCAGT
GCGAAATCTACCACTGAGCACTATCCCCAGGCTACTGTTTAATTTTGGAAGCAGGTGTCTTTGTGTTCCCCAAGTTGGTTAGTTGATCTTTCTTTCCT
TACCCTTACCCTCACCCCACCCCCTCTCCATGGTTTTTCTCTGTAGCCCAGACTGGCCTGGAACTTGGTTGGTCATGAATTTCTGCTCCAGTCTCCCA
AATAGCTGAAACTACAGTCATATGCCCTCCTCTGGCTCTAGTTCCTGGTTTTCAAATAGTTTCAATTCATTGATACAGTTACTGACTCATGTTCTAAA
ATCCACAGTGAGTTTTGTACCTCTGTGCTATTTCCAGGAAATTTCATGTTACATGGTTTTCCCTCCTTTATTAGTAATTTCCTGAGTAACAACTTTAT
AGCAGGCTGTAGAATCCAATCTAGAATGAGATTCAGATAGAGGTAACTCAGCAGTAGAACCCTTGCCTACAGGTCCATCCCCACTTACACACAAAAAG
AGTAAAATTCGTGTTCTCTGGAGGAGCTAGCAGTCCTATAGTACAGGCTCTCCTTTCAGCGGGTCTTCTAGTATAATAGAAGTCTTCCTATAATCAGC
AGTAGGAATCTTCGTATTAAGACCTTTCCCAGGGGGCTAGTGGATGGCTCAGTGTAGAAAAGCATGTATTGTTCTTGCCCAGGACCCAAGTCTGGTTC
TCAGCACACATATTGAAAGGAAATAAAACTTGAGACATGTGATTCATGTAATTTATGTCAAATAGCCCAAAGAGTTGTTTGTGAGCTTTGAAACCTGG
GGCTGAGAACATAGCAGAACAGGCCAGGACATGCCCGGGCAGGCCCGTCGTTACATGTCCTGACTGGCCTAGTGCCTACCTATCTCCCACCCTTCTGA
TAGTCTGTTAATGTTTAAATGGACCAATCATGTAAAACCGCGCCAATTCCTCCCCCAGCCCCACCCCTTTTCTATAAAAGTCCCTAGCTCCCAAGCCT
CGGGGTCGAAACCACTGTCTCCTGTGTGAGATACGTTTCGAACCGGAGCTCCGCCATTATGGCTCCACCATGTGGTCGACACCTCTGTCTCCTGCGGG
AGATATGTGTCGGCCCGGAGCTCCATCATTAAACTACCTCATGCTTTTACATCAAGATGGTCGTCTGTTCGTGATTCCTGGGTGCGCGCTGAACGACA
ATTGAGTGGGGGTTTCCCCACTAGGTTCTTTCAATATCAGGCAGCTCACAACTGCCTATAACTCTAGCTCCAAGGGATCCAAATAAGTCTCCTTTGGG
CACTTGCCCTCATGTGCACATACTTAAAAACACAGGCATATACATACACACATAATTAAAAATAAATCTTAAAGACCTCGCCAAGTCTTCTAATATAT
AGTAGGCATTCAGTTTTAATGAATACTAATGTAATTTAATTGTAGTTTGCATTTGACTTTGTTATTTTTTTTACTTGACTGATTTCTCTAAAATATAA
CAAATGAATTGAAGGTTTTAGATAACAGTGGTGGTCTAATTGTTTTCTTTTTAAGATTTTCACTTATTTTTATGTGGATAAATGTTTTCCTGAATGTA
TTCAAGTGCATGCCTGCTGTCCAAGAAGGACAGAAGACAGCACCAGATCCCTGGAACTGGAGTTACAGGAGCATGGGACCCTCCATGTGTGGGAACCA
AGTTCAGGCTCTCTGCAAGAGCAATAAATGCTTCTCACTGCTGAGCGGTCTCCAGCTACCACTCCTCCCAGGTTTTGAGATAGCATCTTATTTAGCCA
GACCTGAAGCTTGCTAACTAAGGCTGACCTTGGACTCCTTACGTGCCTGTCTCCACTCCCAGGTGCTGGCTGACAGCGTAAGCATAAACCACCACGTC
TAGGTTATGTTTAGCTTTTGGATGCAATCATTTTTATTATTTTCAAGTTTTATTTGTGTGGGGGGGGGAGAAGGAGGGGAAGGGAAGGAACTCGGGAG
GGAGGGAGGGAGGGGAGGGTCCATTTATGCTTTTTGTATGCAAATGCTTGTGGGCAGAAGAGGATCATTCAATCCATTCTCTTGGAGCTAGATTACAA
CAGTTTTGAGCTGCAGATGACTGAGTTGGGAAGTAAACTCAGGTCCTCTGAAGAACAGCAAGCACTCTGAGCACCTCTCCAGCCCCTGTCTTTTTCTA
TTTTTCCTCTGGTCCCCCACACACCATCGCTACATACTGTACTCCTGAACTATTCCCTTACTCTGACTTTTTGTTGTTCTTTTAATTTTTTTCTTATA
TGTATTTTTTAATACTGTAAGTCCTTTCTAGATTTAATCATTTATTTTATGGGTATGGGTGTTTTACCTGCATGTATGTCTGTGTACCATGTGTCTGC
ACACAGAGGCCAGAAGAGGGCGTCAGATCCCCTGGGACGGGAACTACCTGTGATTGTCAGCCACCACATGGAACCTGAATCTGCTCACCTATAAGAGA
AGGACTGCTAAGCTGTCCTCATAGCACCTTGAATGCATTTTTAAAAAGAAAATTAGGACAATAATGGATATTCCTTCTGTGATTCTTTTCCATTCAAC
ATCATATTAGAAACATTTTTATCATTAATTTTTCTATGATGACACATTACTGGCAAAAAGTCCATGGCTTGCTTTACCATTCCTTTAGTGTTGGGTAA
ATAGGCTTATAGAAGGTTGCTCACTGCCTTTCCCCCTCCTCCCTCAGGAACAAAACCCCTGTTTTGTTCCAAAAAGTAGGGCTCTGGTGGCAAGGGGA
ACTTTCAGACTCAAGAAAGGAAGCCTTTTATCCCAGGCCAGGGAATAATTTCTGATTGTTCTGACCTTGTTAAAGTAAATCCTGCCACTTGTGATTAG
CCTCAGGATATCACAGGCTCATTCTGCCTGGTGATGCATAAGACAGCTCTCTGGTGCTATTCTTGGTTTTTGTTTGCTTGGCTGCCCTATGGGCAGCT
TCCTATGTGAAAAGACTCTTGGGAAGAAAGGTAAAGCTTTGGGCTTTGCCTTATTTTGTTGATGCAGAATCTCACAAAGTCCAAACTGGCCTTGAGTT
CATTTTATAGCCAAGGGTGAGTCTGAACACCTACCTTTGTCTTCCCCAGGCAGCGGGTGCAGCCTAAAGCTTTTGGACATCTATTCCACCCTGTCAGT
TAAATGTGGATGGGTGTCCCAAGGCAGCCATTTTATGGCTGTGAGGACTGTCCTGTGAGTCACAGAGGAATGAGTAGCAGGAGCGGGCCTGGTTTCTG
TGACAGTGTTGAGACCATCACTGAACATCTGTGTCTCCACCTCTTATTTAATTAGAATGCTAATTGGCTTTGTGGCTGTGTCTTAGATTTTAACTTTT
AAATTATATTTTCTGTGTGTGTGTTTATATGCATGTCTATAGAAGTCAAAGGACAACTTATAGGAGCCAGTTCTCTCCACTATGTGGGTCCTAAGAGT
TGAACTCAGGTTGTTGGGCTAGCAGTAAATTCCATAACCCACTGAGTTATCTCTCAGGCCCAGCTAATTAGATTTTGTATCACTATACTGAAAATGGC
ACAAACTTATTTTCCATTTTTTGCTCTGTACATTGCAGAAGTTAGTGTCTTGTAGAATATTCCTTGAATTTACAAAGATATATTACTGTATGGGGTGG
AGTGGGTGGGTGTGTATGCTACAGTGCTCAAAGGACACCTTTGTGGAGTTTGTTGTGACTTTCCACCTTTATGTGAGTACTCTGATTGAACTAAGGCT
CACAAAGCAAGCCCTTTACCTACTGGTCATCTTATCCGGATACATCGTTGATGGCATGACAGACTTTAGATCATAGTCTTAAATGTAAGTTCTTGATT
TTCTACTGAGAATTTTGTATGCTAGTTTCTATATAATTTGTTTACCATCTATGAATTTGAAATCAGTAGGGATAAAGGGAGATAGGAACAACTAATTT
AGATGGATCACTCAGGGCAGGCCTTTCAGAGATGAGGATATTTGGGTAGAAACGGGAATGACCTTCAAGAGAAAGCACAGCTGAGTGGGAGTCCCTTG
TGCGCAGAGGGCACAGAGAATGAAAGCTCAGTGACAGGGACAGCCCAGACAGTAGCAAGGCTGGGCGGGCGGGGTGGGGAGGAAGAGCTGCTGCAGGC
AGACAGACAGACTGACAGATAGGCAGACCTTGCAAACCATAGTCAGGGAGTTTAGATTTGTTCTGGGTGTGATATGAAGCACCTGCGTTGTTATTTTA
ATTGCTATTTTTTCTGATGTGGGAGGCTTTGGGAGGAGCAGCGCTGCCATATTTGTGTCTTACAACAATTACTCTGACTGTTATGTAACCTGGGACTG
CTGGAGCCTAAGACAGGCACAGAAACCCCACTTAGGAAGATTATCTAATAATGCTTGAGACCAGGTGTGCTTCTCCAGAGGTTTTCAAAGTATTGAAG
GATTTGCATAGATTTTGCCAGTGGAGCATGCCTGTCCAAAAGTAAAATCTTCGAAATGCTCCAGAGGCAAATCTTTTGGAGTATCACAGTGGCATTCA
CTGAGGTTCAGATTCTGCAGTATCTCAGATTTTGGAATTTGGTAATCAGTCTGTGATATGGATAAGAGATACCAGTAACAAATTAGGATGGACAGATG
GAGAGACAGACAGATACACATACACAGACACATACAGATACGCAGTATCTCTCCTCCTCCTCCTCCTCAGATAAATGAAGGAGAGTACCCACCTTATC
TGCACTCAAGGGTTTTCACCCTTCCGGTCTCCTCTTTAGTGTCAGGCTTATTAAACTTTCCCATGTTTCTTAACTAGATAAGCATGCATTTCGTATAC
TCCTGACTTTATAAAGGGCAACATCCCATACTCTTTTCTATACTCAAAATGACTCTGAAACAGTTTACATAGTTCTTTCTCATTCCCTTGCTATAGTG
TCATGGCGTTGCTTAGCATGTGTGTGTGTGTGTGTGTGTGTGTGTGTATGTGTGTGTACACATGTGTTCATTGTCAGTATTTTACTATGATAATGTTG
AGAACTTTTGCATTGTCTTTACCATGCAATTTACGTCTTGTCTTGTCTTTTGACAAGGTTTCTCTGTGTAGCCCTGGCTGTCCTGGAACTTACTCTGT
AGAGCAGTCTGGTCTTGAACTCAGAGATCCTAAGTGCTGAGATCAAAGGCATGCATACCACCCAGCTTTTTTTTTTTTTTAGATTTGTTTTATGTATG
CGAGTATTGACCAACATGTATGTATGTAGCCCATGTATGTGCCTGGTAGCCCAGAGGTCAGAAGAGGGCATCAGGTACCCCAGAATTGGGGCTAATAG
ATGTTTGTAAATCACCATGTGGGTGGGTGCTGGGAACTAAGCCCAGGTCCTCTGGAAGAGCAATGAGTGCTCTCTCTTACTGCTGAGCCATCTCTCCA
GCCCCAGTGCTCCTTCATTCTTACATGATAGAAACATGTCTGTCTGGATCTCTAGTCAATGAAATTTCTTCCCTACAGCCCATGCATTCTTGTTAAAC
TTCATTTTAACCTGTGGACCCCTCATTCGTTTGTCACTTGTTGGGTACTATGTAAGATTAGATCTAAGTTTACTATTTTTTTGAAATGGCATCTAACC
CCAGAGCTGTTTCATTAAAAAGCTCATTACCCCAGGGATCTCCTCCACCCTGTTTTTATGAGATGAATTTTTATACTGTAGTGCAGGCAAGTCTCCAA
TTCAAGGCAGTTCTGCTCTAGCCTACTCGGTGCTGGGGTTCCAGGATGAGCTGCCATGCACAGCTGTTACCTGGTGATTAGAGATGCCGCCTTTATTG
TGTACAGATGTTCATATGAACTTGGATCTGTTTCTGGACTACTGGAAGATTGAGGCATGTGGCAGGGCACAAGGATTTTATTTTATTTTATTTTATTT
GAACTGAAGTTTTATTATATAACCAGGTTAGTATAGAAAGTAGTCTAAGCTAGCCTTGAATTCATTGCTCTTTTTGCCTGAGTACAGGGGTGACAGGC
CTATGCTACTTCATCTGACTCAGGACATTATGTTTTTGTTTCTGTATTTGGTTTTGGTGTTGTTGTTGGTGGTGGTGTTTTGTTTTTTTGTTTTTTGT
TTTGGATACAATATGTCATGTGACCTTGAACTACTGGTTCCTCACTGATTCTTCTGCTTCCATCTCTTAAGGGCTAGTATTATGGGTATGGGACATTA
TGTATGTTTATGGGTCCTAGGATCAAACTCAGGACCTTGTGCACGGTAATCAGGCACTCTACCAACTGAGCTGCAACCCCCAGCTTTTTGTTGCTGCT
GTTGTAAATATTATTCGTGGGGGTGGGGGTGGTTATTCTATTTAGACCATATAGTTCTCAAAGACACAAAAACCTTTAGTTTTAAAATAAGCCTTAAA
GCACTAGAGCTGGGCAGATATCAGCCCTCTATGCTCTTTTGTCTACTCCCCTGTCAATAACCCTGAGATATCACTTGTGTGTTGTGCCTGGGCTGCTC
CTGTTCTAACAGGCTGGCCCTCATGGCTGTGCTCTCACCATTCATCTCCCCCACAGCGACTTCTGCCTTCTCCCCCTCCTCCTTGTTGTGGTCCCTGC
CTGAGCCTTCTATCTGCCCTGCCCCTCTCTTTTGTACAGCCCAGGCTGTAGGCATCTTTATTAACCAATCAGAGATAACTTGGAAGCAAGTTTTATAC
AACTAAAGCTGGCATATGTAAGGATCTGTTCTTCTCAGAGCAGTGAGATCTTGGGGTATAGAGTTTAGCATTTGAATATCAGCCTCACCAGAACAACT
GCCATACATGTTTATTTATGTATTCAATTGTATATCCTATCTTGCTGAATTCTTTCACTAAGCTACTTTTTATCATTGATTTTTCAGGGGTGTTTTTT
AGTTATACAATTATATTGTCAGCTTTTAATTTTTCCTTTACCCATTCTACCTCAATATTTAGATACTTAGAGGTTTTTTGTTTTGTTTTGGATACTTA
GCAGATTTTAAAGAAGGTTATTAATTTTACCAAATTTAAGGATAACATGCAAAAGCTCGAAGATTACATTTACAACTTGGGACTTACAGTGAGATATA
CCATATAGCACATCATTTAGATTTAGATCAAATGTGTTTATGTATTGTCATGGTCATGAAGAACACTTATCCTCTAATTGTGTCTCCTAGTGTGTGCT
GTATCTATGGAGCTTAAAGATTAACCATCCTAAAACATTGTTTCTGCTTCGAGGAAATCATGAATGCAGGCATCTTACAGAGTACTTCACCTTCAAAC
AGGAATGTGAGTATACATCTCTCCAGAACAGTCAGTTATCTCAGGTGCTGCCACACTCAGTTAAGATCATACCTTCAAAATGAAAAACTCAAGCCAGG
CTTGGTGGCTCTTGCCTGTAGCTCAGCGCTCGGGAAGCTGAGGCAGAAGGATCACCACAACTTTGAAGCCAGCTTGGGCTATAGAGTCAGACACTGTT
TAAAAGAAGATGGAGAAGAAAAGTTCAATGCTTTTACATTGCTTATCAGAGAATGAGTTTGTTTAGCACACACACTCCACTATACTCACATGGTAGTC
TCCATTTGACAAGGTTATAATTGAGTCGCTTCACTAGACAGGTTATAATAGCTGCCTTCTTCCCAGTATAGCTTGAAAGGAAAGGTCAGAATTATTAA
AGAAAATTGTAATCCAGGCTTGATAGACATATCTGTAATCCCAGCACTTGAGAGGTGGAGACAAAAGGATTAGGAATTCAAAGCCAGCCTTGGCTATG
TGGTGAATTCAAGGTCAGTTTGAGCTATATGAAACCCTGTCTCAAAAAAACAACAAAAGAGACAGGATCTTCAGATCATTCCAGCCCCAGAAACTTCC
TGAAATAAACACACCATCAGATAATAACCAGGAAGGGTTTCAAAGTAGATTTACTCTTGGTAGGGATTTAAATTGGTGTCAAGTATTTGGACAGCAAT
TTTGAAATATTTGTGAAGATTGGTTTTGTATTTGTGAAGTATACATTCTCTTCTCTCAGTATTTTGACTGCTAAAGCAACTGTCAGGAGTTTAGTGAC
TCCCACAGGGCTCATGTCACAGCAGAAGTCAGGATTCTCTGTAGGCTTTGCTCAGCAGGGACCATGGTATGTGAGGACTGTATTAGAATCTTCCTCTG
AGGCTAGTAGTCGTTTAGAGTCCTTGCAATGATGGATTAAAGTCCTTTCTTCTAGCTCCCAAACTGGGGGTGAACGGTGGGGGATGTCACTCTCACTC
TCAGTTTTTAAAGATTGCTTTCAAGTTTTTGACATGGTTCCCAAAGGTAGTTCCCAGTGTTGACGTTGGCTTTCTTCTGGCCCTGCCTGGATGAGTCT
CTGACTTCTGCAGTCAGAGCTGGAGGAACCCTGTGCTCTTTAATGGTTGGTGTGATCAGGCCAGGTTCATCCAGAAGACTCTTCCCTTTCCGTCCGGT
GAAGTGTAACAGTGAGAGGCTCATCGTAGTCAGAGTCCCACGGACTTGAAAGCTATACAGAGTGGAAGCCATTGGAAGTCATGTTACAATTTGTCTAG
CACAGATGGACAGAAAACAGTAGATATTTGCTTCTTAAGGATGCTCATTGCAGTGTTAATTATAACAACAAAAAAAATTAGATGTCAATAAAGGGCCA
ATTAAGTACAATATATGAGTATCAGAAAGGGGTGTCGGAATACAGAGTTAGGAAACTTTATGGAGTCCAGTCTTTGCTTCACCTCATTTATGTGGATT
TCCAAGGTCGATCTCAGGTCTGCAGGCTTACACAGCAAGTGCTTTACCTGCTGAGTCATCTTCCTGGCCCCCAAAATAACCTTTATAGCAATACTAGA
CCACTGTTTAAATATTAAGGGATCTGTGTGTGCTATATGGAGAGATGCTTAGATGTGCTATTAGCTGAGAAAAACCATTGGCTGACTTGCACATTGTG
CACAGAAGTCATTGGCACTAGTGTTACTTCTTGTGCTGGGTCAGAGGATCTTATTTTAATGCTTAAAATATTTGAATTACTTTTTGCCACTATTTTGA
ACTACTTCTGTAATTTTTTTCAAGCTGCTAACTTAAAAACAATGCAATTGCAAACCAAGTATTTAAATGGCTTTTATTTGTTTTCTTTTTTCTTCTTT
TCTTTTCTTCCTTTCTTCCTTTCTTCCTTTCTTTCTTTTTTTTTTTTTTTGTGACAGTGTTTCTCTGTAAAACATGGCTATCCTAGAACTCAAGCTGT
AGATCAGGCTGGCCTCAACCTCAGATATCTGCCTGCCTCTGCCTCTGCCTCCTGAGTGCTGGGATTAAAGGTGTGTGCCCCCACTGCCTGGCTTAAAA
AATAAATAAATAAATAAATAAATAAATAAATAAATAAATAAATACAGGGGCTACACAGGAGGAAACCCTGTCTCAAAAAACCTAAATAAATAAATAAA
TAAATAAATAAATAAATAAATAAATAAATAAATAAATACAGGGCTACACAGGAGGAAACCCTGTCTCAAAAAACCAAAATAGATAGATAGATAGATAG
ATAGATAGATAGATAGATAGATAGATAGATAGATAGGACTGCAGGACTAGTGAGATGCTGAACACTTGCTACTGAGTTTGACGATCTGAGCAGTTCCC
AGGAAGCAAAGATCTGATTCCTGCACATTGTTGTCTGCCTTCCACATTTGTGACATTATATGTGTGTGAGCGTGGACACACAAGGTATGAAAGAAAAG
CATTACTGTGCCAGTGTCCCTGAGAACTCCCAACTCCTTGACTATTTGAAGATTTTAAAGTACTTTGCAGTTTCTCTAACATATACTGGTTTTGCCTA
AAAAATTACATAATATATATAAAACAGGCAACAACGGACCTGGGATCTGCTGGATGGAAGTCAGCTACTTGCACTTAGAGTTAAGTGAGCAGCAAGGA
CATGGCCTGCAGGTGCCTTGCTCTTCGTGAGGTGGCTGTGCTGGTTTCCATTTGACAGAGGGTTTTCTGCATTGCCCTGGCTATGCACAGCTCATTGG
TTTAGGCTCCTTGTTTGGCTTGGTTTTTAAGGGACATTTACTGTAATTTCCCGCCCTGTTCTCCCCTTTTAAGCCACAATTTTTATTTGTTCTTTTAT
GGGTTTGGTGCTGGAGAGTGAAGCAGATTTTGTTTGTGATCAGTATAATTGGTCCTGAGTATTTTTAGAAGATAGAACAATAGACTCAGGAAATGCTT
TTAAGTTTTTTATTTTGCCCAGAATTCCTCACCTCATGTCTCTTGTCTGCTGGCGCTCAGTGTGCTCCTAGCTGTACTCCAGCCTAGTCACTCCGTCT
GCTGACATACTAATGTACACATATAAAATGTTATTCCTATATGAGGAAAGAGCACGTATGTGTGTTCACTGACAGGCTGATCTCGTCTGTGGGCCACT
GTGGCTTTTAGAATCAGAACCATATAATTACTGCTAAGTGATGCTCGGTGGCAGAATGTGCAGCTGCGGGCTTTTAAGGAGTGTGGTTTTCTTGTCTC
ATATCAGTTTTATTTATTTATTTATTTTTGAAACAGTTCTATTTTTACTTTTTGCTTTAAGAAAGAGTCTGAGTTCCCCAGGTTGGCCTTGAATTTTT
GGGCCTCCTCTTAGCTCCTACATAGCTGGGATCACAGGCTGATGTCACCAGGGTCACCAGGCCTGTATCACATTTATTCTTACTTATACTGAGAGACT
TATTTTTCAATTTCTCTTGTTTTCTCTAAAGCACAAGAAAATTTGAGGGCTGGAGAGATGGCTCAGTGGGCACTGACTGCTCTTCCAAAGGTCCTGAG
TTCAAATCCCAGCAACCACATGATGGCTCACAACCACCCGTAATGAGATCTGATCCCCTCTTCTGGTGTATCTGAAGACAGCTACAGTGTGCTTACAT
ATAATAAATAAATAAATCTTTAAAAAAAAGAAAATTTGAATAACAAACAAACAAAAAAAGAATAACTGATATCTTTTGGGAGGTGTTTTTAAGACAGG
GGCTTGTTAGACTACCCAGGATGGCCTTGAACTACCTCAGTACCTACTGCTGGCCTCAAACAATTAGTGGGCCTTCTGCTTCATCCTTCTGAAATTAC
AGGCATGTGTTACAGAGCCTGGCTAATAGTTAACTATCTGCCTACTGTTTCTTATTGTTGTGTACTTAGAAAGCAAGAAGTGTAGCGAGTGTCTTGAC
CCCGTCTCATAGTTTTGGGTCCAGATATCTCAAGAGGATTAGTTTGTTGTTCCCCAAACCTCTTTTTGATAGCTTACTGACAAGTTCCAGAAACTTTT
GTAATTTTCTAAAGGACACAGCACTGTTCTAAAGAAGAATACTGTTCGTCACAGTATTTGGAGTAGCAGATTGGTGTGGTTAGATTATGGCATATCCA
TAAGTCTAGAGCTGTATAATTTGTTTTCTTTTTAACTACCAAGCTGTTATCATAAGAAAGACAGAATTTTATCTTATCCCTGGGGATTTCTCAGCTAG
GCTAAAGAAATTGGTTTGCTAATGGCCTGTACCATTAGTCCCCAGCACAGGTGACAGTGGACCCTGGGGACTTGGGGCCAGAGAGAGAGGAGAGACAA
AGACAGAGGGGGAATCTTTACTGAATGCAAATGAGAATTAAGAAAATATATGCTCAATAGCTAGGTTATGGTTTCTTATACATGATGCTGACACTCAA
AACTTCCTGGAATTCTTTTACAATTTTGTTTTATTGCTGGCCATGGGTGGGGTGATGTTTGCCTGTAATCCCAGTACTTAGGAGATGAGTTAAGATCA
GATTGGACTATATGAAACCTTTTAACTACAGTTTGTTCCATCCTGTTGTATTATTGGCAAGAGTTAAACCTTATATAAATGTCTATAATATAGACATT
AATTTTTCATGAAAATAAAGCTCTTTAATCAGCTTATTTGACCTTTTGGAATGATATATTTTAGTATTTAAATTCTATTTAATTTAAACTTAAAAATA
GTGGTGGGCATAGTGGCACATTGCCAGTAATCCCAGCATTCTAGAAGCTGCCAGTTCAAGGCCAGCCTGAGCTTCATAGTAAGATCCCATGTTGACAG
TTCACAGAATGAGGGTATAAACATTTGGGAGAGCACTTGCCCTGCATTCTCAAGGCCTTGGGTCCCAACCCCAGCACCATAGTTTGGAAGAGTCTGAG
AGGGTAGTTAAGAATGTGGATGTGACTTCTCTGCCACTGAAGCTTGCAGTTATGAGCACTGCGCCCAGTTTTCTCCCCTTTTATGTAATTGTGTGTTA
TATTTATATGTATGCTTATGTGTGTGTCTGTGCCTGCTGCTCTCAGAGACCAAACAAGGGCATCTAATATCCTGGAACTGGAGTTGCATACCATTGTG
ATTCACCATGTGGGTGGGTGCTGGGAACTAAACCCAGGTCCTCTGGAAGAGCAATGAGTGCTCTCTCTTACTGCTGAGCCATCTCTCTAGCCCCAGTG
CTCCTTCAATCTTACATGGTAGAAACATGTCTGTCTGGAACTCAGTCATGGAAATTTCTTCCCTATTGTGTGGGTTTTGAGGATTGAACCCTGGCTCT
ATGGTAGAGCAGACAGTGCTCTAACAGCTGAATCACCTCTCCAGTTGCCTTCCTCAGTTTTTAAATGAACATGGGAATGTGCCTACTTCATAGAGATT
CTGCGAAATGATCACTTAACACATAGTTATAACCATTGTGTGCCAGGTGTAAATACACCTCTATCCTCATAGCATCATGACTTATAGGGATTATCATT
CCCATTATATAGTCGGGAAAACACATGGAATGGAAGCATTTGCTCAAGATTCCAAGCTAGAAAGCTAACAACCAATCTAAGGACCAAACTAAGAGAGT
TAGGTTACAGGTCATTTATCTGACAACTGACTGGCTCAGGCATTTAGCTCACGGATATATACTGTGGTCACTATACATGTAAGTGTACTTGCTATATA
CTTATAGACATGTACTCATCAGAGAACATTAGAAAAAATAGGACTGGCCAGGTGTGGTGGCACACGCCTTTAATCCCAGCACTTGGGAGGCAGAGGCA
GGCGGATTTCTGAGTTCGAGGACAGTCTGGTCTACAAAGTGAGTTCCAGGACAGCCAGGGCTACACAGAGAAACTGTCTCGAACCCTGTCCCCCCCCC
AAAAAAAAAAAAAGAAAAAAGAAAGAAAAAAAGAAAAAATAGGACTATGGTTACTGTCTCTAGTCTAATCCCTCGGGGAATGGTAGCATAGGACGGCC
TGTTTGCGCACATACTAATATGCCGTACATGTCTTTTCACCTGATTTGGCTTTTGTTCTCCTGTTTTGGTTTACAGAGTGGCTCGGTACTCTGTGCAT
ACTTGCTGTCTCTCCACAGATCTGGCATTGTTTGGAAGTAAGCTAAGGACTTTTTCTGAGTCATTATTAGGATGAAAGGCATACTAGATGATGGTGTG
GACCATGAATGCCACTTAGACTCTTACCTATACTCAAGTTTAGGATTCTTATAAAGGAATTTTAATTGACATTGGTAACAAAGGTGATTCTGCCTTTG
GATTTTTAGGAGGAAATATCTAGCACTGACCACATGTTAAAGATTCTTTGTTTGCTTTTCTTTTGAACAGATGTTATACTCACAGCTGGATCTGCTTT
TGAAATAGCAAAGTGAGGCTGTAACTGTAAGGAGGTGCATCCCTGCACAAACTGAGCTCATGTTAAAGTTAGATATGCTATTGAATACAGTCTTAACC
GCCTCTGTGCCAGTGTGAAGAGAGCCCTCAGATTCCATTAAACCCAAACCTGAGTCCCTCAGTATCAGCCACAAAGAGAATGTCTGGATTGTTATGAG
AGGGAGGCCTTCTCACCTTCGTTTATACTTTCCTTTGTTTTGTTTGTTTGTGTGTTCGAGATGGGGTTTCTCTGTGTAGCCATAGCTATCCTGGAACT
CACTTTGTAAACCAGGCTGGCCTCTAATTCACAAAGATTCATTTGCTCCTGAGTGCTGGGATTAAAGGTGTGTGCCACCATGCCTGGCTCCAGTTTAC
AGTTTCCTATATGGACTGTAGGGTTTCCTTATGTATACTGTCTTAGTCTTCTCTACTAGCATTCACTCTAAAGGCACAGCTTGACTTTAAATCCAAGG
TCAACTGTATAATCTGTTTGTTACTACTCTAACAAAAGACCTGAAGCTGTGAACCTTATAAAGAAAAGAGCTTTGTTTAGCTTATAGTTTTGGAGGTT
TCAGGGTATGATACCAGCATTGGTTTTGAATTGATGAGGACTTCATGTTGGGAGAACACATTAAAGAGAGAGAGAACACACAAGGCCAAAGATTAGAG
AAGAGCCCTACTGGAAGAACTACTGAGTGTCTCACATCTTCCTTCTGAGGGCAGTGCCCCCAGTGACTTCCCACTAGGCTCCACCTGTTCAGGTTTCT
CTTCTTAAAGGTTTACCCTGTGTGGTAACATCCCACACTGAAACCCACACATCCCTTGAGAGATACATTCTAGTCATAGCCACACCATAACACCAAGA
AGGAAAATGCTGGTTTTTTTTTTTTTTAAGCCCATCTTTAGAGTTCTTACCTGTTACTTCATGTCTTTTTCTTCCTAGAGAATTGTGGCACACCTACA
ATCTGCCCACTTAGGAGTATTTCCTTGTACCATAGTTCCAACTTGACAACCACATCAAAATATAAAATGCAGGAACTCGAGAGATGTCTCAGAGGATA
AGAGCCCTGGATTCCCTAGAACAGAATTTAAGGAGAATTGTAAGCTGCCATATTTGGGTACAGCTAGGTAAAGCTTCCAGCACCCACATAACAACAGG
TAGCTCCCAAGTGCCTGTCATCCAGGTCCAGGGTCTACTGCTGCTTCTGAGGGCACCTGCACAGTGCACATAACTTAGGTAGACATACACAAATAAAA
AATAAATTCAGAAATGCAAAACAGTCTATGTCTATGTAAAGTACACTAGAGTAGTAATAATTTTCCTACACATTTTTTAATGTAAGGGCTGGAGAGAT
GAAGCTCTTAGTGGCTAAGAGCTGCTCTTCAAGAGAGGACCCAGATGGCAGCTCACATCTCTCCTGAACTCCAGTTCCAGGGGATCCAGCACTCTTCT
GGCTACCTCCCCAGGCATGTGCTCAGTGCATGAACACACACACAGGCAAACTACTCATACACGTAAAAATAATCTTTTTTAAAAACAGAAAAAAATGA
GCCCAAAGGTTTCTGATACAGAAATAGTTTTCAGTACTGTGGAGACAGTTCCACATGGGGTGGGGCTGGGATCTACTTTGCCTGGGAAGCCCCAGGGA
TCAGCAATGCCAGCATTGTTTGTAAACGCTTAATGGCAGAATTTGAGGATCCTAAAAGGACCCTCAGGAGCCTAAAGCCGTAACCTTCCTCCAGGTAA
ATGATGCTCACTAATAAGACATGGGCAGCCACCTGCCTTATCAGATTTCCTGGGCAACAGAGCTAGAAAGTTAATCTAAGGTCATACTTATCTTTAGA
AAGGGAAGAGTGTTCGGTTAGCCCTTGTGTGCAACTCTAAAAGGACATTTTCTCTGGGACAGGGTCTTCAGACTTGCCTGTAACCAGTGGCTTCCGAG
CTGGCTTCACCTGGTTAGTCTCTTCAGTATGGCACTGTTCATCCCCTGACTTTGTCCTCTTTGGGTAGGTCGGATCAAGTATTCAGAGATGGTGTACG
ATGCGTGCATGCACACTTTCGACTGTCTTCCTCTTGCTGCCCTCTTAAACCAGCAGTTTCTCTGTGTACATGGAGGAATGTCTCCTGAAATTACTTGT
TTAGAGGACATTAGGAAAGTAAGTAACTTTTTACTATTTTCATAGAGTGTGCTTTTAAATTTTTGTTCTTTATCTTAGCCAGTGACTACCTATATAAC
ATATAGCAATTGCTGAGGTTAAATGAAAATACAGTCAGAGGTAGAAATACGGGACTGACTCATAATCTTGTCACTGTCAGAGAGCTGTTAGTAACACT
TGGAGAAACATTCTTTCTCTTTTTAAAAATTAAAGTTTTGTTTCGTTTTTTGAAACAGGGTTTCTTTCTATAATTCTCGCTGGAATTCACTCTGTAGA
TCAGGCTGGCCTTGAACTCTCAGAGATCTACCTACTTCTGCCTCTGAGTGCTAGGATTAAAAGTATGCGCCACCACTGCTTGGCCTTAAAATTAAATC
TTAATAGCTAAAAGCATACTCTTCTTACAGTAGAACTCTGAAACTTTAAAAATCATATTTGTATATTTTAAACAAGGTCTCTCTGCAAAGCCCAGACT
AGCCTTGAACTCAAGACAGTTCTACCTCCTCCACTGTATGGTATAATTACAGGACTTGTGCCACCATGCCCAGCTTTAAAAAATTAAATTATATTCCA
GCTTGCAAAGAAAGAACATGAATGTGAAGCCTTGGCCAGGCCCCTTACCCTTGGAGTACATTCTTTCTGTAAATGAGGAAATTTGACTGAGTGCCCTG
AGCTCTGGCAGCTGTGAGGCGCACTGCCGTTTGCATCCTAAACAGGATGGTTGGTTGCTCCATGTGCCTTACACTCCCTTATGTGGTAGTTATTCCCA
AGACTGTGTCTTCATGACTGAGGGAAAGGAGCAGCCTTGTGCACGTGCACTTCCGCACCTTGATCCCCTCAGTTCCCACGGGGCACAGACTCAGGATT
TCCTTGAGTTCACAGCAGTCTCCCAGTGAGCAGTGAGCCGGTGTGTGTGTGCTGGCTTTTCAGTACCCACCCAGCATGTCGCCTTTAGTAGATTTTGT
ATTTAACCACTGTGATCAGCTGTGGTAAAAAGCTCCCCTCTCACTCAGCAGAAGCCCTCTTCTTGACTCTCAGATGTTCTGGTGTGTTCAAAAACAAT
TATTTATATTCTTTGATTTTTTTGTGGGGGTGTTTTTAATTAAAGCAGCAGTAACTGAAAAAGTAAGTTAATGACAAATATTAAAAGTAAGTTAATGA
CACCACTGCATAGAATGGAAAGAATAAAAGGCTTTTTACAAAAGAAGACATCTCTATTAGAACGAAGTGAGCAGTGGACAGGATGCCTGCCTGGCTCC
CCGCCCTCCTGTAGCTCTGCTTCCCATAGAAAATAGAATTTAACTTTAGTCGTCACAATTTTTTGTGGAGGCTGATTTCACATTTGAGGTAAATTTCA
CTAGATTAGCATGGGAGGAGAGGCCCTTTTCTACAGGAACAGAGATAAGATACTTGTACACATTTATTGACGAATTCTCATTTTCTCAAGGGACCATC
CTTGGGGCTCACGGCCTCATCTGCAATACTTTCCTGTAGGGAGAAGGGGACTTTTAATCTGCTGCTTCCAAAAGCCTTTTACTTTCCTGATTTTTTTT
TCTCTTCTTTTGGCCCTTGTGTGTCTTCTAGTTAGATAGGTTTTCTGAGCCTCCTGCTTTTGGGCCAGTGTGTGACCTGCTGTGGTCTGATCCCTTAG
AGGACTACGGCAGCGAGAAGACCCTGGAGCACTATACCCACAACACTGTCCGAGGCTGCTCCTACTTCTTCAGGTAAGCTGAGAACACAGTGAAACAA
ACTGGGCCCCATTTTACTTTTTCAGTTTTTCTTTAATTACATATGTGTCTATACACATGAATGCAGGTCCAAGAGAGAGACTAGAGACATAAGATCCC
CTGCAGCTGGAAATATAGGCAGTTGTGAACCACCCAGTGTGGGTGCTGGGAACTTAGGTCTTCTATAAGAAGAGCACATACTCTTAACCACAGAGGCA
TCTTTCTAGCCCACTGTGATCCATATAATGGAATTATTCTTTTCTAAATTAATTTTTTTTTCTTTATGAATGTTGTATCTGCATGTCTGTCCGTCTAT
GTACTAAGTGTGTGCCTGGCGCCCTCTGAAGTTAGAGGAGGGTATTATATCCACTAGAACTAGAATTCTGGAATCACCATGTGGGTGCTAAGAACTGA
ACTTGGGTCCTCAATAAGAGTGGTAAGTGTTCTTAACCACCGCGCCATCTCTCAAGCCCTAGTTTTATGAAATTAATCTTGCCACCACACCTGGTATT
ATGAAAGTTTTCTTAATGGCTCAGGTCTCAAACAAGAAGTAGAGACTACTGTTTAGTCTCATTCTCTGTGTTCTTATTTCTCTTTTTTCTCTGTCCCC
TCTCTCACCTCCTTCTCCTCTTCCTTTCATTTTGGTGTTGGAGAGCAAACCCAGGACCTTACACATCCTGGGCAAGCACTTGTTCAATCCCTTAATAG
CAGGAAAGCTTTAATATGTTCTTAGACATGAGATCATAAGAAGTACAGATTAGTGAACACAGTTAAAACCTCTCCCAGTATCTCACTGTTATAAGCTC
TAAATGATTTATTATTTGTTTCTAGATATCATCTAGATACTTCCTAACGATCTGACTTTTAAAAGATTTTATTTCTTTATTTTATTATTTCTATATTT
TATAGCTGTCTTCAGACACACCAGAAGAGAGCATTGGATCCTATTACAGATGGTTGTGAGCCACCATGTGGTTGCTGGGAATTGAACTCAGGACCTCT
GGAAGAGCAGTCAGTGCTCTTAACCACTGAGCCATCTCTCCAGCCTCTTATTTTTATGTGTGTCGGTGCTTTGCCTGTCTGTGTGTGTGTAAACTACA
TGTGTACCTGGTGCTTGTGGAGACCAGAAGTAGGCATCGGATCCCTTGGATCTGGTAGTTACAGATGATTATGAGCCATCTGGGACGGGGAGGGGTGG
GGGCTGCAAACGAAACCTGGGTGGGTCTTCTGCAAGAACAGGAAGTGCTCTTTAGCACTGAGCATGTCTCCTCCCCCTCCCTCTCTCTTTCCCTCTCC
CTCCCCCCCTCTTTTTTTTCTCTCTCTCTTTCTCTCTCTCTATGTATGTATGTATGTATGTATGTATGTATGTATGTATGTATGTACGTACATACGTA
CGTACGTACGTACATAATCCCAGTATTTGAGAACAGGCAGGCTGCACAATGTGAGTCCAAGGCCAGCTTCATCTACATAGTGGGTGCCAAGCCAGCCA
GGCCTACGCTCTTAGGGTTACTGTTACTGTGACAAAACACTATGACCAAAAAGCAAGTTTTTTTGGGGGGGGGGCAGGGAGGGTTCATTTGGCTTATC
AGATCACAATCCATCATTGGAGTAAGTCAAGACAGGAACTGAACTAGGGCTGGAACCTGGAGGCAGCTGATGGAGAGACCATGGAGGGTGCTGCTTAC
TGGCTTGCTCCCAGCTTTTGGAACTTCTGCTATTGACCAAAACAAACCAAAACCAAAACCCCAGAACACTGATCCAAGCCTAGGACTTTTCCTGGAAG
TTATTGTCCTAAAGTTCACATGTGAGCTCTTGGTATGGTTTGCCAAGCCTAAATAAATAAATAAATAAAGTAGAGAAAATAAAGACATTGAAATCTAA
ACTAATTTTAATTCCACTGTTTTATTATGACAAATTTAGTTCTTTAAAAATCTTGCTAACTGGTTAAGATAATACAGTAATTAAATGATACATATGTA
TAAAAGAAGCCTTAATTTGCCCCTAAACATTTTTAAAATAATCGTTTTGAATTCTACTCTGCCATCTATCTTTCTAGTGACTTATTTACTGAAACAAT
TTAACTTTTCCCTCTTATGATGTTGCTTAAGGACTTAGCATTATTGGCCAAATGTCATTTGTTACAGTTCCTGTGGTCACCTGCCAACACCTCTTACA
ACACAGGCCTTCCACCCAGAAATTCTCCTCCTAACACTTGGCTTGCAGCTATCCCCATACTCACCTGAGTTCCCTGCATCCCAGTCCGTCCTCACAGA
GCTCTTATAGCATTGGTGGACACACTTGGCAGAACAACAGATGCACCTGAGCCAGCCCATACGGCGCAGGCTTACAGCTGACATTAGAAGTCATGCGC
TGACTCTTGTTTTTAGTCTTTTTTTCTCTTTTGTTTTACGTGTAGGAGTGTTTTGCTCACATGTATGTTTCTGTACCACATGTCTTCCTGGTATCCAT
GGGGGCCAGAACCCCTAGAACTGGAATTACAGATGTGGGTGCTAAGAACCAAACCCAGATCTTCTCCAAGAGCAGCTCTTAATCACTGAGCCATCTCT
CCACCCCCACCTCACAGTTTTGTTTCTGTTTCTGCACTTTTCTGTCAGCAGTCTTTTTCAGTGTTGATAATGGTATCACACAAAGTGCTGTCCAGTGT
CCGTGAGCAAAGCAGACTGTAGTGTAACTTACAGAGATTTGTCAAGTGTGAGTTGTGTTCCTGGCCATGAGTTCAATGTTAATGATTCAATCTTATAT
TTACATAAAATATCATTATGCAAGAAAACATCGGAAACACTTAAGTGGTTGATGAAAATATGGCCAGAGGCTCCTAGGAATCTTAAACCTCATACGGA
ACCTAGGAACAATGTCTCATGGCATGAATCCTTGGCCGTGGGCTCTACCAGAATTCCACAGCCTCCTACCTCATTCCTAAGGCCTTCTCAAAACTGCT
TCACAGTCTATGAATGGCTAACAGACCATTCATTGTTCCTATGCCAGTCAAGAGGGGGACTTCCTGTTCCGAAATTGTGATAATGTCATATATAGGTG
ATCTTAAAATTATACATTTTCCACTGAGCATAGTAAAAAGAAAATTGCCCCCTTTTTCTCAGACAGGATCCCGTGCATCCCAGGCTGGTCTCAAACTT
ACTATGTAGGAAAAGACAGCCTTGAACTTCTGCTTCTTCTGTCTCACCTCCCAAGTACTGGGGTTACATGCGCAGCATCCCTAAAATATGGTGTATAG
GGGTTGGACCCCGGAATTTGTATGCTGCACAAGCAGTAGACCAGCTTAGTTATATCCCAATTCAAGAAAGAAATACTTTCTAAAAGATAAGTAAGTAA
CAGCTACAAATTCTGATGGTAATTTTATATCCTAGTTCTTCCTGGTTGTCAAACAGAAGTTCAAAAAAAAAAAGACCATTTTAGGAGTATATGCAGAT
ATCATTAGAGTATGGTGGTCTATAATATTTATATCTACTCCTGAGATAGCCATGCATAGTACTATCTTGCTATTGATATTACATATAAAGTTATAAGT
AGATTCTTTTAATTTTTCTAAAGGTTTGATGATTTAAGAAAAGCATGCACTAATTTTGTAGGACCTACTGTATACACATACATGCATACATGGAGTTG
TGATACATCTTCGTATCAAAATACCAACATTTTTGATCCTAATACTAGTAGTGTTAAGGTAAAATAAATTAAAAGATGGAGAAACTTTCTATTCTAAG
GTGTCTTCTTCAAGTTTGATAGTAGTGTGAAATAGAAATCACTGGTTTCTGAGCTTTTTTTTTTTTTTTTTTAATCGAAATTGAATATAATATACTCG
ACAGACAGACATAGAGTGTTTTTCCTCTGGGCTGGAGGAGTTACTCATTAGACCTGCTTCCTGTCCTCTCAAGGATTCTCGGAGAGGCCTGGGTTTAA
GACGCTCTGAGGAGGGAGGTTGTGAGGGAGGAGCTGGTTTCTGAGCTTTTAATAAAAGTTCAGTCGTAGGGCTGGAGAGATGGTTCCGTGGTTGAGAG
CACTGGCTGTTTTTCCAGAAGGCCTGGGTGGAGGCACAATGTTATTGTGCAGTGTTTGAAGATTATCCATGTATTTTTCAAGTGCTGATTTCTCTGCC
TCCACGTCTGGTTGCAATCCTGACATGGCATTGTAATACTTACTTTAAGACTCTCCTCCTGCCTCTGTGATGTGATGCTCCTTATTGATTCTCTGGTC
TGTCAATAAAAGCTGATCAGCCAATGATTGGGCAGAGGAGGGAATGGGGCTGGACTTCCGATGCCAGCCAGGGGGGAGGGTCGGGGGAGGGGAGAAAG
GGCGATGGGAAGTGGGATTCACTCAGAGGAGAGGGAGGGTGGAGGGGACCTTGAGAACACACCTGGAGCTGAGCGACCCAGAGCAGTGCTAAAATACA
AGGATCTTGGGGGTTCGGGCTGGGAGGGAGCCAGAATATTTTAGAGGATTAAAACAGATTAATATTGACCAGCTATTGTGGTGTAAAGCTTGATTAAA
CAAGACTTACAGTATCATCTCATTGATTTGGAAGCTAGTCAGGGTAAAGAAAAAAATTATCACTCTGATGGCTCTAACACCTAGGTTCAGTTCCCAGC
GCTCATGGTGGTTCGTAATTGTCTGTAACTCCAGAGTCATGGGATCTGCCACCCTCCTTTGGCCTCTGTGGGAACTAGACCCTCAGATGGTGCATATC
CATACAAGTAGGCAAAAGCCTCATAGAATAAAGTAAATCTTTAAAATTGCAGAGTCCATAGTCTCTGTAATCTTAAAACAACTCTCAGATTGGGTGTT
CTGTTTCTTCCTATTAAAACATAACTGCTATAATTCCATATTTAGGTTTTTCTCATTTTCTTTTTCAGTTACCCTGCAGTTTGTGAATTTTTACAGAA
CAACAGTTTATTATCAATAATCAGAGCCCATGAAGCCCAGGATGCGGGGTAGGTTGCACTTAGACCTCACCTTCCTGCAGATTTTACTTTTAGTGTAA
AAATGCCTGAGTTCATATCTCCTTTTTCAGGTACCGAATGTATAGGAAGAACCAAGCAACTGGCTTTCCGTCACTTATTACGATTTTCTCTGCCCCTA
ATTACCTAGATGTCTATAACAATAAAGGTAAAGAAGTCCAGCAATATCTTAGTGTGAATTGTTAGTAACTGTGAGCTCTACTTTAATTTTGTGTTTTA
TTTTATTATTGTGTTCATTTGCTTATTTTTATTTTTTGGCTCTGTGGATGGAACCCAGGTCCTTGTGTTTGTGAAGCAGGCACTCTACCCCTGAGCTA
TATCCTGGACACCTATTTTTAAGTACAAAATTCAGTCAGATAAGAAGGTCTTGGGTAAGGGGTGAGGCTTTGTAGTAAAGCATGTGTTTAGCATGGGG
AAGGCCTTAGCGACACTCTCTGGTATCTTACTCTGCTCTGCACCAGCTCCCCAAGAATCTGACATCTCTACCTCTCTCTCCCCAACTCCTCTTCTGAT
TTGTTCTGTTTTGAGACAAGGTTTTGCTATGTAATGCAGACTGGCTTTGAACTTGGGGTCCTCGTGCTTCATCTCCCAAGTGTTAGGGTTACAGGTAT
GCACTCCCATTCCTAGATGAAGAAGAGATCTTATTGGGAAAAAAAGGTCAGTAAGTTTAGAATGTAAGCTGCTGCTTGTCCTTCATCTCCTGAGAGAT
TAGGCTCTTTGGGAGCATATTTGTTTAGTATTTTAAAATGGGATCTCACTGTACAACCCAGACCAGCTTCAAACTCCCACCCTACTTTGGCCTCCCTA
GTGCTGGGATTAAACATCCACCATCATACCTGGCTCTCCATTAGGTGGGTGGTTGGTTGGTTTGGAGACAGGGTTTCTCTGTGTAGCCCTGGCTGTCC
TGGAACTCACTCTGTAGGCCAGGCTGGCCTAAATTTGAAGATTCACCTGCCTCTGCCTCCTAAGTGCTGGGGTTAAAGGTGTGAGCCACCACTGCCCC
AGTTTTGTTTCATTTAAATCTGAGACTGAGAGCTGGAGAGATGGCCCTGTGTTTTAAGAGCACTGGCTGCTCTTTTTACAGGACCTAGGAGAAGTTTC
AGGGGACCCAACACCATCACACAGACATGCATGCAAGCAAAACAACAATATGCATAAAATTAAAAATAAGTTGTTTTTTAAAAATCTGAGACTATCCT
TCAGTGGTTTTTCTTTCTCTTTTAATCTTGTGCTCTGTTAGGACCTAGAGCCTTACTCTTGTTAGGAGGTATTGGGCGCTGAGTTCCACTCCCAGCCC
TGATCACTCACTCCATAAAAACAGATCCTCCCTCCCCTAGCCTTAGCTTTATCAGCTTTATCAACTGCTCCAGGCTGTCCTCTACTCCTGACTGTCCA
ACTGGGCTCTGCACCACCTTTACTGTGTTCTCATTTCTTTTCCCTAGATTTCATGGGTATTAGCAGCTTGGGTTTTTTTTTTTTTTTTTCTAGTTTCC
TTTCTTATCAAGTATACATACTTTGTTTTAAGCATCCTCAAGGATAGTTGTGCCCCTATGCCTCGTTTTGTGAGGCACTCTCTTCCTTCTCCCTTTTT
AGCACCACTGTTGAGATCATCTGTGGAAAGCCTTGGGGCTCCCAGTGTGCTTAGCTGAGACCTGCACTCACATACGCACTTGCTAATACCTTACCAAG
AAATACCTTTATTTTATTTTGTTTTATACATATTTTTTGAGACAGAGTTTCTCTATGCTGCTCTGGCTGTCCTGGAAATTACTATGTAGACCAGGCTG
GCCTCAGACTCACAGAAAGCTGCTGCTGCTAACTTCCAAGTGCTGGGATTAGAGACATATGTACTACCACCCAACAGTTTACAAATGCCTCTTTAGTT
CTGAGAAGAACTCTCGCACCTTCTTCCTCCTATGGCTGCTTTTCCTGTCATTTGTTTTCTTATTTTTTTGAGACCTAGGCCAGATGACCTTTGAATTT
GCTTTGTTGTCTACAATGGCCATGAAATTCTAATCCTCCTGCCTCCATTCCAGAATACTGGAATTACAGGCATGTGCCATGTCTGACATCTGTGGTGC
CAAGCCTCATGCATGTTAGGCATCGCTCTTCACGCTGAGCATCTTTGGAGAGATCTTTAGGCTCATTGGTACCCAGGCTGTATCTTTGTTCCCTGATG
ACCTAAAACATCCTTAAATTGGGGTTTTTATTCTTATTTCTGTCTCCCCACCCCCTATCCCCTGGGAACATACTCCAAAAATTTCTTAAGCTTCCATT
TTTAATCATCCACTGTTGAGACTAACTTCTTTTTCCTGTGACAGAATTACTGATATATTCATGTCAGCTATATAAAATGCACACAGCATGAAAATAAT
GGAAGTGTACATGTCAGGTAAAGTTAGGCTTTCAACAACTGGAAATCCTGTTTGTGTGTGCTTTATAGTAGTCTGCCATATTCATTTTAGACCATTAG
TAACTTAAAGTTTAGAACACTAGTCTATCCCACAGCAAGGAAACAGGAACCCAATGTGACCTTGGTCTGTCATTTTTAAAGAATTGTGTTTCATGTCT
CAGTAAGAATAATTGAAAGAAAGCCAGGTGTAGTGGCACATACCTTTATAATCCCAGAACTTGGGAGGCAGGAGGCAAGTGGATTGCTTAAATTGAAG
ACCAGTCAGGGAATTCCTGACAGCTAGAGCTCTTGGGTGGTGGTAAGAGAAGAGAAGAAAAGAGAAGAGAAGAGAAAGAGAGAGAGTGTGTGTAAAAA
CAAACCAACAAAAAACCCTAACCAAACAAAAAAGAATAATTGAATAAATAGTACAAATAACAACAAACGAGACAGAATAAGGAATTTTTAAAAAATAT
AAAGAAGCCTTTTCCCCAGCCCCCTCAGCAGTTGGTTAGGGGCTGCCCCTCACCTAAGGCAGGAAGGTGGTGGCTGCAAAGACACAAAAGTTTCTAGC
CAGATGACACAGGTCTCTGATTCCAGCACTCAGGAGGCAGAGGCAGAGGCAGGAGGATCTCTGTGAATTTGAGGCCATCCTGGTCTACAGAGCAAGTT
CCAGGATATCCAGGAGTACACAGTGAAACCCTGTCTCACCCACAGTCACAGGTCTCTGGTGTTTGTCAACTCTGGGCTCCTGTTTGTTATAAAAAGTG
GAAAGTACACGCTGAGGTACAGACTCTGAAGATGATCAGACAGAGCCAAGCAAAATTGGTTATCTTCAATAAAAACTGCCCAGCTTGAGAAAATCTGA
AATAGAATACTATGCCATGTTGGCCAAAACTGGTGCCCATCACAAAAGTGGCAATAATATTGAATTGGGCACAGCGTGCAGAAAATACTACAGAGATG
CATGCTGGGTATTATTGACCCTGGTGATGCTGATATTATCTGAGGTACCAGAACAGATTGATGAACAATGAACAAGGAAAGCTTTGCCAGAGCTCATT
TAAAAGAAAGTATACAGAAAATCAGTAGGTATGCATCTATGTGACATGGTTACTGTCAGCTGTTTATAGACACTCAGAATGCTGAGAATAGAAGACAC
TTTTTTAATTGATTGAGATTCTACATTCTACCCTTAAACATTAATTATAAATTATTTTGCCATTTATTGCACTGTGAGTTGATAACATCTTGGAGTGT
TTATAAAAAGCTCTAGCTGTGGCTCATGACTTGCAAAGGAGTCTCCGTCTGTTCGATGGGTAATAGTGCTATTTGCCCTTAGAAATTGTGACTGTTAA
CTGGCCAGTAAATGGAAATTATAGGGAGTTTTCTATGTTGAATCTGAAGTTTCTTCAGTAGCTAGTGCACAGTAAACCCTTGGTTTTTCAAAGAGCAG
TGTCTTCTGTGTTTTGATGATGTGAATTCCCCAGGTGTCTTTGTTCCCGGTGATAATTAATCCCCAAGAGAATGAACCCTTTAAAATTCTTTAAACTT
GTCAGGCTGTCCTGAATTTACAAATGGAAAATAAGTCATTGAAAAGTCACTGTAGTAGGCTCACAGGGACAAAGCACAAAGCTGCTATGCTGGGGTTC
TCTGTGCACACATGTCTTGTGTGTTTACTTTTTAGCTGCAGTGTTGAAGTATGAAAACAATGTCATGAACATCAGGCAGTTCAACTGTTCCCCACACC
CCTACTGGCTCCCAAACTTCATGGATGTTTTCACGTGGTCTTTGCCTTTTGTTGGAGAGAAAGGTAAGAGAATCCCTGTGTGCATCTGAACACCAGCT
CTCTTCTGATGAGACACCTTTATATTCCCAGAGTTTATTGGAAATGTGTGCTAATTTTGTTTGTTCTCTACAAAGATTTTAAAAATTTTTACTTATAA
AAAAATCTGACTTGTGTGTGTGCAGGATGTGTATGTGTGCAGTACTGGGTGCAGGATGTGTGTGTGTGCAGTACTGGGTGCAGGATGTGTATGCGTGC
AGTACTGGGCGCAGGATGTGTATGCGTGCAGTACTGGGCGCAGGATGTGTATGTGTGCAGTACTGGGTGCAAGATGTGTGTGTGTGTGCAGTACTGGG
TGCAAGATGTGTGTGTGTGCCTGTGTGCATCTGAACACCAGCTCTCTTCTGATGAGACACCTTTATATTCCCAGAGTTTATTGGAAATGTGTGCTAAT
TTTGTTTGTTCTCTACAAAGATTTTAAAAATTTTTACTTATAAAAAAATCTGACTTGTGTGTGTGCAGGATGTGTGTGTGTGCAGTACTGGGTGCAGG
ATGTGTGTGTGTGCAGTACTGGGTGCAGGATGTGTGTGTGTGCAGTACTGGGTGCAGGATGTGTGTGTGTGCAGTACTGGGTGCAGGATGTGTGTGTG
TGCAGTACTGGGTGCAGGATGTGTGTGTGTGCAGTACTGGGTGCAGGATGTGTGTGTGTGTGGAGAGGGAAGAGGACTACTTCGTGGAGTGGGTTCTC
TCCTTTCACCTTTACCTGAGCTTCAGGGATTGAGCTCAGGTCACCAGATTTACATAGGAACACCTTGATCCAGTGAGCCCTATACAGAGATTTTTAAA
TGGCATTAATCTGTTCATAAATATTAGTCTATTATGGTTTTTCTTTATGTGAAATATAAATTTAAAACAACTGTCTTGTGTGATCAAGATCTTAAAGT
AATGTGGCCACTATTCTATCTCTTCAATGAATTATCAGTGCTAACAGAATGTAGACTGAACTTGTTACAGAGGTTCATGTGCACAATGGCACAAGTAA
GCAGTACAAACATAAAACACACTTACACAACTGAGCTGCGCAACGCAAACAGACCCACAAGTGAGCAGTAGAGACAGAAAGTGAGCCCGGTGGGTGGA
GGCCCGTTGTGATTTCTTTGTAAAATGGAAAGTTTTATATGCATATCTGAGCCAAAGGTTCTGAACAGCTATGTAATAAAATCAAGTAGTTCTGTGAT
AGACTCCACAGGTTCTTATCCAGCCAGGACAAGTGTGGACAAAAATTCTCTCTGGGGCTGGAGAGACGGCTCAGTGGGTAAGAGCACTGACTGCTCTT
CCGAAGGTCCTGAGTTCAAATCCTAGCAACCACATGGTGGCTCCCAACATCCATAATGAGATCTGACACCCCTCTTCTGGAGTGTCTGAAGACAGCTA
CAGTGTACTTACATATAATAAATACATTTTTAAAAGAAAAAAAGTTCTCTCTGAACCTTATGGTCTACCCTTGGTTCTATTTTCATAGCTAATTATAA
TGTGATAAGTTGTTTCGGTCTGTGTTTACTTAATGTATTTTAACTTATGCACATCCAGAAGTGTCCTATTAATTGTGCACACACTGTGTTATTTGCCA
CTTAGTGACAGAGATGCTGGTCAATATTCTCAACATATGCTCGGATGAAGAAATGAACGTAACCGATGAAGAAGGTAAACTTCTTACTCAAATAGAAG
CTGATGGCTTCTGATTTCTTTTTATTGGTTTTGGTCTTACTAGAAAAGACTTATGTTCATTGTAAAAAAAAAAAAAAAAAAAGTTTGCTGAGCAGTGG
TGGTGCATGCCTTTAATACCAACACTTGGGAGGCAGAAGCATTCAGATTTCTGAGTTCGAGGCCAGCCTGGTCTACAGAGTGAGTTCCAGGGCTATAC
AGAGAAACCCTGTTTCAGGAAAAAAAAAGTTTGATAACTATAGGAAACCATAAATAGGAAGAAAATAGTCACTATTGTTCTGCCATCTCAAATGTCTG
CCATCAATGCCTATGTGTAAACTGTCTTTATCTTTACATAGATCTTTTTTCTTCTGATTTTTGTCACTTACAAAAATGAGGTGACATGCTATCATGTC
TTTATATCTATTTCTTGTTTTCCTTTATAGTCTAGTTTTTCATGGTTACATAGTTGTCACATTCATCAATCACTGTGCTAACCAATATTATAGATGCT
GCTGGGTATTATATTTTCCCCCACAACTTTAGCTTTTAAAACTGCTACAATTTGTCACCAGATGGAAGAGGCACACACCATAATCCCAGCATTTAGGA
GGCAGAGGTAGATGGATCTCTGTGAATTCAAAGCTAGTCTTGGTCTACAGAGTAAGTTCCAGAGCTACACAGAGAAGCCCTGTCTTGAAAACCAAACT
AAAACCTGCTAACAGTTTCAAATTTGTATAGATAAATCACTGCTCATTCTATCAACAGCCAGACTTTGTTATATGCCTGATGTGTGCCAAATATTGTT
TATGCCTTTCTTCTTAATAAACATACATTGTACTATGGAAGACAAACAAGAAATAAATACAATGCAGCTACTAATGCATGCTATGGAAGAAAGTAGTG
CTGTATTAGTAACTCTGAGAGTAACATTTACTTGTTTACTTTTATAGGTTAAATGTCTAGAAACTGCAAATATTTCAGTATTTAATGCTTAATCTGAT
ACAGATAGCATTTTTGTTTGTTTGTTTGTTTTGTTTTGTTTACACAGGGTTTCTCTGTGTAGCCCTGGCTGTCCTGGAACTCACTCTGTAGACCAGGC
TGGCCTCAAACTCAGAAATGCGCCTGCCTCTGCCTCCCAAGTGCTGGGATCAAAGGCATGCGGCACCACCACCCCATCCCCACCCCACCCCACCCCCG
CATAGATAGCCTTTTAAAGTCTATGTTACTCTGTATCTCTTTGTGTGTGCTACATAGTTCATAATGCTTTTCCTAGTACTGTTTATTTTCTTATTGCA
GTTTACACACACACACACACACACACACACACACACACACACACACACAGTGCTGTAGCTAGAACCCAGACATTGTGTAAGCTAGACTAGTGTTTACC
TCTGAGCTGTATCCCTAGCCCTAAATTTTTAATTTGTACTTTTATGATGACTAATAATTGCATTTTTCTTATGTCTATTGACCATATTATTTTCTGTT
TTGTGAATGTAAATTCATATATTACTAGTTTTTCAACTGGATCCTATGCATGTTTTGCTTCCTGTTTGATATGTAAGAGATCTTCGTGTTGTGTCTTA
GGATAGTAATGCTGGTTATATGTGTAGCTAAGTGCTTTTTTTCCCTTCTGATTTCTATCACTATTAATTAGTTTGTTTCCTTTAGAAGAATTAAGTTT
AAATTAATTTGTAGCCAGGTGCTTCTAGTGTGTTGTGAGTGGTTTGATCTGAAAGGTATGCTCTAACTTGTCTGAGGATACAGGAGTTTTTGGGGTAT
GGTGTCACATGATCACAGACCTCCAGTTCATATTCTTTGCAGGTCTATGTAGTTCACTTAACTATTGCATAAAGTGTTGTATGGTGGAGAACTACTAG
CTCCCACTGGGAGTCTAGAGGGGATTCCAAAACATATGTGTTATTAGATTGTACTGGGAGATTATAACAGATGGATTATAGCTTATATCTTCTGGATA
GGAAATTGGTGAATGGGGCCTGTCCTTTTGAGGAAATCTGAAAGGAACTTTAAAAAGAACATAGGGAAAGTAAAATATTTTAAAATGGATTTAATAAA
TTTTATATGCACATCTAAATAGAAATTGAAATGGTTGTAAACTATGATCTGTGTTAAGAAAAACTTTTTGGGCTTGTGAGATAGCTTAGCTGACGAAG
GCTCTTGCTGATGGGGCTGATGGCCTGAGCCCCATCCCCAGGCCTCATGTGGCAAAGGAAACCTAGTTCTCACACATTATCTATGACCTGCATACACA
GGCTGCACAGTGTGCACTTGGTTCTTTCTTCTCACCAGGTGGGCTCTAAGGCTCTAACTCGGTCCAGCTTTTTGGCAAGCACCCCTACCCACTGAGCT
GAAAATTTGGGTTTTTAAAAAGTAATACATCCTAAGGACTTCTACTTCAGCCCTGCCTGTTAAGACAACTAGAAAAGTTGGACAAAATGTTTTTTGAA
GTTGGTTGGTGGCAGTGCACGTCCTTAATCCCAGCACTGGGGAGGTAGAGGCAGGTGAATCTCTTAAGTTCGAGGCCAGCCTGGTCTGCAGAATTCCA
GGACAACCAAGCCTACAACAGAGGAAAAACCCTGCCTAAAAACAAAACAAAACAAAGCAAAACAAAACAAAACACAAAGTTTTATGAATAGTTTTAAG
TCCACATGAAAGCAGACAAACTAGTAAAGAATTACAAGGTGAGAAATGAAAAGGTCAGAAACGCAGACAGATGAGGTAGTGAGACCATGTCTTGAGAT
CATTCATGAATTTCAAAGGAGTGGGTAAAAGGGTTAAGAGGAATTTTTATAACTTTGTGTACTAATCTTAAAAGAATGGGCTGAGGACTCCCAAGGAG
GGTATATATGAATGTAAGTTTCTGTTCTTGGGACCTCAGAGGGTGACTCTGCAAGAGCAAGGGTGAGCCAGGTGGAGATATACTATACAGTCTCTCGT
CATCATCTCAGCCTTTGGCATCAAGTACTCCCTGGTAGAAAGATCTCTGTGAAATAACAGCATACAACATCCCAGTAGGCAGTCTGTCAAATACAGTG
CATTGGTGACAAGCAAAAATAAGCAGGCACGTGGGGAGCTAACAGCAGAATGAGGAAGAGAAAGGGTTCCCAGAACAGGGCTGGCAGGATGGCTCAGT
GGTAAAGGAACTTACTGCATAGGCTTCACATATTCTAGCTTTGACTTCATAGAGCTAGTATAAGTCTCTGTCCACTGTGTTCACACTGTGGCATGATC
CTCCCCATTACGCATACTCAGGCATGCGTGCGCATGTACATATACATAAGCGCAATAACAACAACAGAAATAAGATTTTAAAAAGTTCCAGCCAAGCA
TGTTGTCATACATCTTTAATTACACCACTTAGGAGGCAGAGGCAGGTGACTGTGTGTGTATGGAATATAGAAGTTCTGGGCAGTCCCAAGATACATAA
TGAGACTGTCACAAACAACAAAGAATTTTCACAGGGCGTGGTGGCGCACGCCTTTAATTCCAGCACTCGGGAGGCAGAGGCAGATGAATTTCTGAGTT
TGAGGCCAGCCTGGTCTACAGAGTGAGTTCTAGGGCAGCCAGGGCTACACAGAGAAACCCTGTCTCAAAAAAACAAAAACAAAACAAAACAAACAACA
ACAACAACAAAGAATTTTCTGAAGCAGCAGAAATTAAATACAGACCCTCCATAGCTCTCAGGCATTGGCTTTGTTAAGCCAAACTTAAAGCAAAGCAA
AATCATAATTTGTTTTTTTGGGAGATTGACTGGCCTGGAACTCACTGGCTTGAAACATCACAGAGATCCTCCTGCCTCAGCCTTTTGAGCCCTACGAT
TAAGGGTATCCCTGGCTTAATTTAAGAAATTAAAAGACCGGGTTAAAAATGTTGGTAGAGAATTGTGAAGTAAAAGAAAGTGTAGCATATTTGATAAA
GCATCAAAAGTGAAATTATAGCATTGAAAAATAAGATGACAAATTAGCAAGTTTGATGCAGTGAGACAGGGACTAAGTGACCTGGAAGGTAGGTCAGA
GCAAAGCTAACTGAAGCATGGTGAGAAGGGAGTGCAGAGTCAGCCAAGAGCCTCAGAACACGCGCTGCCAGCCCAGCACATGGTGCCTGGAGTACCAG
AGAAAGCGGGAAAGGCAGTCTCTGAAAGGTCCACATCAAAGCAACTTCCAGAACTGAGAAGACACGGAGCTGTAACTGAAGAAGCCCTCAGCAGAGCA
GGACAGACACTGACAACAGCTTCTAGCCTCTTGTAGTGAGGTCCACAAGATGGAAGATGGATCTGAAAACAGCTTCCAAATGGGGGTCACCCCTAGGG
AAGCCGCCGCCGAAAAGCAGGTTCTTCATGGAGATAGGGGAGCAGGGCTGGAGTAACACACCAATGTGTGAAAACTAACACCCAGTACTCCAGAGTCC
AGCACACAGCAAATGTGTAAACAAGAAAGGCCAAATAAATATTTGTAGATACCTCTCCAACCCTCACATTTACTAAGAAAATTAGCAGGAAATATATA
CTAAATAAAATTCTAAAGGACAGTATTTAAGCAGAAGGAAAATCCAAGAAAGAAGTTTAGAAACACAGGAAGGAATGAAGAGAAGTAGAGAGCGCATG
CACAGACAGTGTGGACATGGATTCTTGTCATGCTTGAACAAGCAGAGCATGTGCCAGTACTATATAAGCAATACTGGGATGAATGCATTCAAAATGTA
TAGGCCTGAAAATTCTGATCACCCCAGCACCTCTGCATACATGATAAGCCAGTGTTCTGAACTATAGCCCCAGCCGTTAGCCTTATACTTCAGTACGT
AACAAACTCATAGTGCAATCTCTAGGTTCAGCTGGCTTACAGGTTCAGAGGTTCAGTCCATTATGATTCTAGTGGGAAGCATGGCAGCATCCAGGCAA
GCATGGCAATGGAGGAGCTGAGTGTTCTATCTCCTGTTCCTAAGGCCACTAGGAGACTGACTTCCAGGGAGCTAAGATGAGGGTCTTACAGCCCATGC
CCACAATGACACACTTCCTCCAACAAGGCCACACCTAATAGTGCCACTCCCTGGGCCAAGCATATTCAAATCATCATATCTCTGATAACACTAGCTTG
TATCAAGGTGACATAAAACTAGTCAGTACAACCTAGCCTTGATTGATTCTAAACTTCAGAGGGAAATGGCTAAAGACTTAAAGAACTCCACAGGAGGG
CTTGCTCTATAGATGTCAAGATTTAATATAATGCTACAGCAATTAAGATGATCAATTCAAGGGTAGCTAAGCATTGAAACACAATAGAGAATCCAGAA
TTAAGCTACATGTATATGGATACTTGATTTTATCAGAGGTGAAATTTTACAATGGCTTTGTTTGTGTGAGACCTCAGTGTATAGCTCTGGCTATGCTG
GAACTCACTATGTAGACAAGGCTGGCCTTTAACTCACAGAGATCCACCAGCTCTGGGATTAAAGGTATGTGCCACCATACCTGGCCAATAATGGCATT
TAAAATACTTTGTAGATATCTACTTTTGAAAAAAGTAAATCCTCGTGGATTTATTTTGTGGATCCAAAATACCTATTTTTGTGACTATATTTCATTCT
GATCTGAAAGGTGCGGAGGCTGGGGGTATGGCTCTGCAGTGCAGTACTTGCCTAGCATCCTGAGGTCCTGGCTGCTTCTCTAGCATCTCTACATCACA
GCAACAAACAGGCCAGTAACAGCCAAGCAGTGATGAATTCCTGTCTCTCCAGCCCTCACGACTGTGGCAAGAGAACCACAGGCTGAGGCCAGTGTGGG
TTATGTAGTGAGTTGGAGGTTAGCCTGAGCTTCATAGAGAGTCCCTGTCTCAAGAAACAAACAGCAGAAGTTTTTTAAAACTAAAATGTAGGGAGTGG
GGAGATGGCTCAGTGGTTAAGAACACTGGCTTCTCTTTCAGAGATCTTGAGTTCAAATCCCAGCAACCACATGGTGGCTCACAACCATCTATAAAGGG
ATCTGATACCCTCTTCTGGTGTGTCTGAAGACAGCTACTCACATACAAAAAATAAATGAATAATCTTTAAAAAAAAATCTGAAATGTAAAACAGTGGA
TTTTGTAGTATGTATAATACAGAATTACCTTGATAACTGTGCAATGGAAAAAAATCCTTTAAACGGGATGCAAAAAACACTAGCCACCAAGAAAATTA
TTAATAAATTAGATAAATTGAAAGCAAGAGCTGGGACTGGAGAGATGGCTCAGTGACTAAGAGCACATGCAGCTTTTGCATTCACATGGCTGCTGCCA
ACTCTGGTTCCAGGGGTCTTAATCTGAAGCCTTCTCTGGTCTCTGAGGGACCTGAATGCACTCACATGCATGCAGGTAAAACACTCATGCACATATAT
TAAAACTAAAATAAGCTAGGTGTGGTGGCGCACGCCTTTAATCCCAGCACTTGGGAGGCAGAGGCAGGCAGATCTCTGAGTTGGAGGCCAGCCTGGTC
TACCAAGTTCCAGGACAGCCAAGGCTACACAGAGAAACCCTGTCTCAGAAACAACAACAACAACAACAACAAAACCAAAAAAACCCAAAAAACAAAAA
AAATAAAAAACTAAAATGAAGGTAAGAGCTTTTGCTTATTCAGAGATTTTGTTCAGCATGTGAAGTGGCAAGACATCCAGAAGGATGGGATATTTGCA
GTACAAGCAACAAACAGCCCATTGACAAATGACTGAATTATTCCCAATTAGGATGATGTGGCATGAATAGGCACCTCAGAGAATTTCCTAATGGCTAA
CAAAAATATAAAAATATATTCAGCCCCATTAGTAATCATGAAAATGCAAATTAAAATTATAATAGGATAATACTCTACTTATCAGATTAGTCAGACTT
AGATGAGCCATATCTAGTGTTAGCCAACAGCACATGTAGTAACGGAGTGTCCACATACAGAAGTAAAATGTTATAGATCCCACTCTAATAGACAGTGC
TAAATGTTGTCCAAAGTTAAACATACCCATGCTCTAGAGATGAGCATTCTAATGTCCTAGGAACATGCCAGAAATGTGCATATGTGCACCAAAGACAG
ATACATACAGAAATACATACAGAGCCTCACATGCAATAGCCAAAAGTTAGAAGCAACACAAATGATTACTGTTGTATAGGAATGAATTGTTGCATAGT
TGACCAGGCCTGATACAACTTTTAACACTTGGGAGGCAGAAGCAGGCACATCTCTGTGAGTTCGAGGCCAGCCTGGTCTACTTAGTGAATTCTGGGAC
AGTCAGGGCTATGGAAAGAGACTGTCTCAAAAACCAAAATAAAACAAAACAACAACATAGTGGCACAGTGCTATGGTGGGGCAGTGTGTGATAATGAA
GATGGGCCATGGTGAAAATAAGAGTGTGTCTTACTGAGATATTGTCACCCCAGTGCTCAATTGTCCAACTATATGAAACCCAAAACCTAAAACCTGGG
CCACTGAGATTATTAATTCAAGGGTTGATGAACCCACTGCCAGGCCTGAGCACCTTAGTTCAGTGTCTGGGACCCACATGGTGGAAGAAAAGAAACAA
TTCTTATAAGTTGTCTTCTGACCTCCTTGCTGAGTACACACAAAGAGAGGATAAATAAATATAGCTTGTTAAAGAAAACAATAAAAACCATAAGTGCG
CCGGGCATGGCGCAAGCCTTTAACCCCAGTACTTGGGAGGCAGAGGCAGGTGAATTTCTGAGTTCGAGGCCAGCCTGGTCTTCAGAGTGAGTTGCAGG
ACAGCCAGGGCTACACAGAGAAACCCTGTCTTGGAAAACCAAAAAAAAAAAAAAAAAAAAGTAAGTGCATTCATCCATAAAATTAAAAAGACAGGAAG
AAGATAGTTCTAGTGAAAAGCAAGGGACTTATGTCCTTTGGACCGGAGAGGCTACAGTGGTGAGGTACTATGGGTTGTGAGCAACACTGGTATTGCCC
TGGGTGACTAGTTTTACAGGTATGTGCCTTTTAACAAATCATGTCATGACTAGTTTGCTTTGCATAAATACAGAAAACGTGTGCTTCATAGTAAAAAG
GTAAAAATTACATGTCTACAAATAGAGGAGATCGAACTTTTGGAAGCTGAGAAAACATTCATTGGCAGTGGTCAGTGGGGAGCGAAGCAATTTAGAAG
ATACATAACAACAGCCCTGAAAATGCCAGGACAAAAATCTGCTAATGCAAAGCAGATTTAAAAAATATTAAAAAAAAAAAAGAACCCCAGAAGATCTT
CTCATGCTGTAAGAAAAACACATCCAACGTTAAGATCTGTCTAGGGATGGAACAAGGTAACAGGTGACTTTTAAATGTGTTAGTGTGCTCACTTAGTT
GGTTTGGGAACACTAAAGCCCTGTTTAGTGCTAGGGAATGCTGAATATCAGTACTAGTTGCTAGAACTGAGAATAAAAGAGCAGGCAGGGACACTGCG
CTCACTCAAGTAAAATAGGTGAGAACCTTTGAAAAAACTGTTAAAACTCTGCTTGCCATGGGATACATGTCCCTCTTCTAAGTCTGGAGGCTGATCTC
CAAACGTAAAGGCATTCTGGACTGTAGATGACAAAGGAGAACTCATGTCCCTAACAGAGGACCAGCATGCCATGCTCAGAGCCGTCAGCTGTCTTTTT
CCCCCGCTCTGGAAGTTTGTGATTCCCTCCAAGTCTTACCCATGAGCAAACACCAAGGACCATCAGACCCCTAAGCAATGGCCTTACAAGACAGAAGC
CACAGAAAACAACTTGAAGTCCTGAAGGCTTAAACTTTAAACCTCTAGGAAGGAAAATGTCTACTGACAACTCAGGCATGCTAGCAAGTCAAACAGCA
GCACCGTGTTCCTGCTGAGAGATCTGTGAGCTATTCTGTCCCACAAACAGTATCTTTGTGCTATTTTCATGCTGGGGGTGGGGTGGAAAATAGTAAAT
GGGAATTAATGTGAAGGGTAAACTATTAAGCAATGGGAAGATAGGACAAAACTGGGAATCTGTATTTAGTCTTGGGGAGCAAGCAGTCTCTGAGCTGC
TCCGCTAACAGCCTGCATTTCACACTTGGATTTTTTTCTCTTCCATGTAAACTCCCATCATCCTCCCATGTGTCTGTCTTCATCTCTTTATTTCTTGG
TGTCTCTTCTCCCTGAAGATCACTACATTTCAAGCTGTCCAAAAGGTATCCAACTGGAATGCACTGCCAGCTAAGTTCTTGCTGTCTGCTCTGCCACT
GGTGATTGGTTAGTCGTCTTGACTTGGTAGTGGGCAGCTTCACGTGACTTTCCACCCTCCCCCTCAGTGGGAACGGCTGTCTTCTGTAGACCTGTTGT
GTGATTTAGCTGAGTGGAGAGGTAGGAGAGAGGAGGAAAGCCTGTGTAGCTTTGTCTGACTTTTGGATGCTATTGCCTGGTATATGCCTGAATCTTAT
CTTTCATGTTTGGGCTATTTCCTAAATCTTTAAGAAAAGGCCATAAAGTCTTACATTAACTTACATTAACAGTGTTGCTTTTTAAAATACCTTACCCA
TCCTTGTAATCTTTGTGGCACAGTTGTTAGTAGTATTCTGAAGAAACACAGCTGGCTGGTATAAGGGAGGAAGACTGTAGAAAGAGCGTGGCCTTCCT
AAGTTCAGGACTGCTGCTGCCTTTGGCAGACCAGACCCCGCATCCAAAGGTGGCGCGTAGCATTCTCAAAGGAGATCTTGGCTTGTAGGAGCAAGTGC
TCTATTGCTGTGAGCTTTGTTTGTGTCTTTGTGTTTCATTCCTCACCATGTTTCAGAATATGAAACCTCCTGCTCCCCATAGATGACTCGGAGTCTTT
CTGTGACCTCTTCATTCTCCCCTTCATTTCTTCTCTCCTGCTGATTTCTTCTTGTTCTTTGCAACTTCCCAGGATTGTTAATTGTTTTTTTAAAAATT
TAGGCTTTGTTTCTTTCTCTTTTCTTTTTTTAAGTTTTTCTTTAATTTAAACATAAAAGCACTCCATCTTGCATTTGAAAAGAAAAACCTGTCCTGTT
AATCATATCACTTTAACCCACTTGTTCTTTGTATTTTCTAAGGAGCTACTACAGGTCGAAAAGAAGTCATCAAGAATAAAATCCGAGCCATTGGGAAA
ATGGCCCGGGTCTTTACGGTTCTTCGGTAAGGTTCCATCGTTACACTGTGGGATGAGGGTGTTTAGAAAGGAGGTTACAAGTTAGCTTCACAGCAGTG
TTTTCAAGGATTCAGTCTGATGCTGGAACTGCAAGTCTTCTGGTTGAGTGCAGGTTAAACAGACTCCCTGTGAAAGTGACAGCATTGGGAACATCCAT
GTGGTAGTGGTGTAGATGGAGAGGTTTAGTGTGGATTTTAGAGGTAGATCCCCATGTTCAAACTCTGGCTTTCCTTAGCTGTGTTGTCTTGGGTTTAA
TGTTTGGGTCTTAGATTTTCTTGTCTATAAGCTAGAGATTAAAATAGAACCTAGCTCACATGTTGTTAGTGAGTCAAATGGGAGAAAGTGTGGGGACG
TTGCCTGGCTACCAGTGCCTCTGGTTGAAAAAGGACTGGTTAATTTTTGTGTCTGAAGTGTATGTAGAAACCTGGTCAGATGAAGGTGGGGCCTGTCT
TCCTGTCTCTGTCCCTGTTACCTTCCTTCAGGTCTCATTGCGTTGCTGTGCCATGGCTCCCTGGCATTAACCAGGGCTTCCATGTTCCTTTGCAGGGA
AGAGAGTGAGAATGTGCTGACCCTCAAGGGCCTCACTCCCACAGGCACACTCCCACTGGGGGTCCTCTCTGGAGGAAAGCAGACCATTGAGACTGGTG
AGTATGAAGATGTCCCTTCCTAAGAGGTGTGCCCCCATTACCAGGAGTTGGTGACTTTTGTTTAGAACCTGGTGCTAGAGCAATGGATATTTACCGAG
GGAAAGAGGAATCATTGTCCTTATCCTTCTTGTACACAAAATAATTCAAAGTAGATAGTAATTTAGGGGAAAAAGTGTGAAGAGTAGGTATTAGAGTA
AGCAAAGTCTTCAATAAGAGACTTTTATTATTTTAATAAAAGTAATGCTCTTAAAAGAAAATAGAGGTTATTAAAATTAAATTCCCCTTATGAAAATA
CATAATTAAGAGAAGGAAGAGGCAAGCCACAGAAAAGATTTCTGCACTACCTATGACTGACAAAGGCCTTGTTTCAAAATAGACAATTTATTTCACCA
AAGAAGATGTGAAGATGGCCAGCAAACATGTATGAAGATGCCCAGCATAGTTACTCTCCTGGAAGATGAATATGAAAGCATAGTGAACCACTTCTGCA
TACCCATAAGAATGGCTGGAATGTAGAAATCTGACGAGGTCAAGAGTTGGTGTGTCTATGGAGGCCTGGACTAGAAGTAGGCTAGTGGGCTTACACAT
AGACTCAACACATTGGCCAGCTGGTAGCCAATAAAGCTGAAGACACTTGGACCCATACCCTGCAACTCCACACCAAGGATATCCAAGAGGAATTAGCA
TGCATGTCCACCCGAGGATATGCATAAGCATGTTTGTTTACTGTGGTTTTATCATGGTAGCTCCAGATCAGAAGCAGCTCCCCTGCCTGTTGGTAGTG
AATACATTATATTATATTGATACAGTAGGCTACTGCTCACCCATTGGACCTGTTGCATTGAGGTCAAAGCACAAGCTTAGCTCCTGTCCATCAGTAAG
AAAGAGTGTCACTCTGAAAGCCAGTGATATCTTGAAGAGTTACACATGCTTTGGAAATAACATATAAGTAATCCCTAATTAAAGCACTGATTCAAACC
AGAAATGAAGAGACATAAAATATACTGGGACCAAGCCCAAAAAGGAGTGAAGAAAAAAAAATATGTATATACATACATATATATATATCCCAAGTTAC
TGCACCTATAAAATTCTCTTGCTACCCTTGATTTGTAGATTTGCTCCTATGAGATCTAGCAAAAAAATTATATTAAAGCAACTTTGTGGATCAATAAA
TTTTTAAAACTTCAAGTAAGGAAGATAGGTAGGCATTTCTCTTTTTTTCTTAATCCATGTTACTTCCTATTAACCTAAAACACCCTCATCAGAACTGT
TCACAGAAAATCTCATATGACTAATTAACAAGTAAAATAAAAGTAGTTAAGTATCTTATATTTTTAAAAGCCTGTACATTTCTTTAGAGATATGGCCT
CTAGGAGGTCGACCATCCTTCAGAGGATAGTTCACAGCCCTGTGAAAGAGACTAAGTAGATTCAGTGAGTTATTAAACAACAAAAAAGGAGAGGACAT
GGAGATGGTGGGAGGTGGGTATGATCAAGATACACTATATATGTACAAATGAAGATCCTGCAACCATGGAGTTGAGGGTGCATTGGAAATACTTTATG
GACCTGAGAGAGGACTCAGTGGGTAATAAAAGATACTTATTGCCAAAGTTGATGACCTGAGTTTGGTCCCACATGGTGGAAGAAGAGAACTATATAAA
AGCAGAAAAACCAGCCATATACATAAAATAAAAATCAACCTAAAAAAAGTTAACCACAGTCAGTGGTAACTAAGATAGGTTTGATGATGATGATGAAG
TCAGTAGGTAGCTGGATGTGGCTGCATGCTTGTAATCTCAGCACTTGAGTGGCAGACATGGGAGGATCAGGAAGGAGTTTGATATTCTAGCTACACAG
CAAATTTCAGGGATGGCCTGGCCTATATATGACCCTGCTTAAAAAAAATCGTTTTAAGGATTAATGAGAGCTTCAGTGTCTGTAATAAAGCTCTTGCC
TGGTGTGCATGAAACTTTTGGTTTGATCCTTAGGACTGGGGAAATGACAGAAAGGAAGGAAAGAAAGGAAATTGTTGATGAGATTTTAGAGAAATGAG
GTCATCCTGTGCTGCTTGAGGGAATAGAGCTACTGTAGAGAGAACATTGTATCAGTTCTTCAGATGACTAAATAGAGTCACCATAGATTCAGCACTGC
CACCCTACACGGCCCCCTCCAGTGAGAGAAATAAACTATGTCCTCACCAAGACTTGTATATCAATGGTCATACATGACACTTTCAAAAGAGCCTCACA
TGGCATTCTTCTGAACACCCTAAAGTAGATGCGACTTCAGTGTTCATCCATTGGATAACAAAGTAAGGTCTATACAGTGGGATTCCACATAGCCATAA
CAAGAAGGAATGCACTCATATGCATCAAGGCTAGGCTATACTGCATAGTGAGTTCCACGCCAGCCTAGCCTACGTAGTGAGAGCCTGTCTCAAAACAG
CAAAAATAGTGACATACACACTTGAAACAATGTTCTAGCATAGGAATTATATGTAAACAGTTTCATAATTTTTTTTCTTACCATGTCCCTTGGTACAT
ACAGACTGCATCTTAGTAACCAATGCAAAAAACAGTTCAGTGAGGGCCCTAAAATTTTAATACTGGAGGTCTGTAGAGATGGCTCAACAGTTAAGAGT
ACTTATGGTCTTTGCAGGGGACCTGAATTCGGTTCCCAAAACCACATAATAGCTCAAAGTCATCCTTGCCTCCAGTTCCAGGGGGTGTGACATCCTCT
TCTGATCTCCACATATGCAGGCAAAACATTCAGACACATGAAATAAATAAATCTAGATTTTTTTTTTTTTGAAATGGGATGCTGGTATATACTTTACC
ACACACATTCAAACTCCAAGTAAACCACCACTCTCCGTAGAGTTTTAGACGGAACTCTGGTTCCCACCTTCTCACAGCTGCAGAGGCAGCACCCTGCT
CTGTGAGAGGTGCTGTCCCTCCTTGTGTGGCTTATCTGGCCCGTCTGAGGTCAGAGAGACTACAGCAGTGCTCTAACAGGAGCCTTCCTAGGGGCCAT
TGCAGGCACATGGGTGCTGGCCGCCAGATCCTGCTCTTAAATTGCAGTGGGAGGTGAAGGAGCTTCGCAGCTCTCTCTCTCTCATAGTGATGAAGGAA
ACCTCACGGTGGTCTTGCTGCTGAGTGGCCTTCCTTCCTTCCTTAGGAAAGTTGCCCTTGGAGGAGCTGGCAGGGCTCTAATGGGATCTCATTGCTGT
ACCCTGGAACCATTTCATGTGTTACAGCTTGACTGCCTGACAGAAAGTGGCAGGGGGAAGTTTCCCTATTGGTGCTCATTTTAATTGCTTCCATTTCT
GGCACCGGAAGGGAGAAGATTGCTTTCTTAGTAATTGACCTGCTTTCTTGCACAAGGAAAAGAAGCCATATGAGGTGTTAGCGTGAGCCTCTGCCATG
TCCTGCATCTCAGATGGGTACAAATCAATTAGAGTTCAAATCAATGCCTAGCTGTTCACCTCTAGACCAGAAGTAACTCTTCACTTATAACCTATTTT
TCTCCCCTTCCAAATGCTAGAGCTATTCAAGTACTGTCCTATTCTTTTACTATTGTAATTATTAAATGGTTAAGGACAGTTAGGTCATGCGTAAGTCA
TTTAACAATACTAAGAAGCAAGATTAAATAGGATTTTTAAATAGGACTGAGCAACAGCTCGATTGATACAATCTGAGCAGTTACAAGCTCAAACATAG
TTGTGATATTAAATTATCTGATTTAACAAGTCATTTTGATGTGTGATGATCACAAGACACAACCAGCAGTTGATGATTTCAAATTCCCATGCCAACAG
GAGTGTTAGAACTGTTAGTGCCTCCCCACCTCATGCGCATGCTCCTGAGGTAATCCCCTCCCTTCTCTCTCTGGGTGGGACCCTGGCTGCTTTACTGT
CAGTTCACTCATCCTTTCTCTGTCTTCATTTTTGTATGCACTGCTCCTGCCTTCTTACAGCCAAACAAGAAGCCGCAGAGGAGCGGGAAGGTATGGCC
ATATCCCTCAGCCGGAACACCCCTGTGTCTGTTCACCAGCCTCCAAGCTTCATTGGCATGTCGGAACTCACTAGCATCTGCAGAACCCTGACAGGAAA
GGAGAGGATGGCCCTCAGGGTGGCCACATGCCAGCCTGTTTCTCAGATGGCCATCACACAGCCTGTGTCCTTTCTAAGTCTGTCAGTTTTATGTAATG
ACTGAAGCTTGTGCAGCTGGTAATGACAGAGGTCAAATGATTTAAGTGGTTCCTTTCTTTATTAAGAGAGTTACCGCCGGGCAGTGGTGGTGCACGCC
TTTAATCCCAGAGGCAGAGGCAGAGAGGCAGAGAGAGAGGAGAGGCAGAGACAGGCGGATTTCTGAGTTTGAGGCCAGCCTGGTCTACACAGTGAGTT
CCAGGACAGGCAGGGCTACACAGAGAAACTCTGTCTCAAAAAAACAAAACAGACAGACAGACAGAGAGAGAGAGAGAGAGAGAGAGAGAGAGAGAGAG
AGAGGAGAGAGGAGAGAGAGTGTGAGTGTGTTACCTGGGGCTGGAGAGATGGCTCAGGGATTAAGAGCACTAACTGCTCTTCCAGAGGTCCTGAGTTC
AGTTCCCAGCAACCACATGGTGGCTCACAGCCATCTGTAATGGGATCCGATGCCCTCTTCTGGTGTGTCTGAAGACAGCGTACTCACATATATAAAGT
AAATAAATCTTTAAAAAATAAAAATAAATTTTAAAAAGAGTTACCCAATAACTCAAAGGTTAGCAGGAGTTTCCCTAAGACATTTCAGCTTAGGGTAT
TTTGTTTCTATGGAGACATTTTAAATCATACTGCTTATGAATTTTTAGATAAAAACCATTGGAATTCACAAATCTGCAATGTAGATTTAGTAATCTCA
TTAGATAAGGTGATTCAAAGGGGAATTCATTCCTTGGCAATACAAAGTTGTATCTGGTTGGAACAGCTCAGTGGTAGAATGTTTATCTAGAGTATGTA
AGGCCCAGGGTTCAAACCCTGGTACCACAATAAAAAATTTCAGTTTAATCAGATGGGAAGTATCTAGAAGCTATGTTGATGTTTTTCATCTGTGTTCA
GCTCCTGGCTGTGAAGATAAAAAGCTGTTTGAGCAGATAGGTCTCCTGTCACTTGTGCATGGCTAATGCCATGTTTGTGCTTAAAGCAGCTATCTGTG
TGATAGGATGTGCTTTCCTGGTGTTGCCTTTTCTAAAAGTGAGCAGGGTTTTCATAAGAAACTTAACTTCCAAGTGTGTGGAGGCCAATTAACACCTA
ATCCTTTTTATTTCTGCCAGGACTTGTTATACTTTTCTCTTCTCTTCTCTTTTTATGATTTAGTTATTTGTTTTACTTACCTGAGTATACTATTGCTC
TCTTCAGACACCCCAGAAGAGGGCATCGGATTCCATTACAGATGGTTGTGAGCCATAATGTGGTTGCTGGGAATTGAACTCAGGACCTCTAGAAGAGC
AGCCAGTGCTCTTAACCACTGTACCATCTCTCCAGCCCCAAGACTTGTTATTCTTATGGTAGTTTTTACCACACTTGAGCTGCTTAGAAGCAGTGTTT
GTGAACAGAGCTGATTTAGCCACGTGTTGCCTGTCAGCAGATGTGTGTTCTGTAGCTGCTTTCCTGTGCGTTCCTAGGAGATTGCTGTGTGCTGGGAA
GCTTGGTTTAAGCTGCTAACTTTGCCACCTCTCTGAGCACTTCCTACCCCCTCCTGTCTTTCTGCAGCCATCAGAGGTTTTACAATTGCACACAGGAT
CCGAAGTTTTGAAGAAGCCCGAGGTCTAGACCGAATTAATGAGAGAATGCCACCCCGAAAAGAGGCTTGATATCATCATGATGCAGGGAGGATGCACT
CACACTCGCATCCGCCACACCCACAGGCGTCAAGAAGGACCGACCATGGAAAGAAAGCCCTGTAATGACTCAGGGCCCTGTGCAGCACAGATGGGTCC
CAACCCTATGAGCAAATCACATTTATTTATACTGGAAATGAAACAGGAACAACTCAAACAACTTAAACTTGGAGGTGCATTTGTAATTCAGTCGCATT
TATTCTGTAAGAAAAATGACCATTTTATAAATTCTTCTAATTTATGTTCAATATATATATATATATAAAATACTTTTGTTTTGTTTCCCTCCCCTTGT
CCTAATTTTAGGAACGAATCTGATTGGTTGGGTGTGTGTGTGTGTGGGTGTGTGTGTGTGTGTTGAAATCTTATGCTATAAAGGGGACCTTCCCCTAA
TAATAAGGGCCTTGGAAACCTTCACCCTAGATTTCTGACTCATACTCCTAGTTAGCCCTCTTCTTGTTTGGGGAGGTGATTTTTTTTTTTAATTTATG
ACATAACTCGAAAATGTCTTTCAATAGCCAGGCGTGGTGGTGCACACCTTTAATCCCAGCACTTGGGAGGCAGAGGCAGGTGAATTTCTGAGTTCGAG
GTCAGCCTGGTCTACAAAGTGAGTTGTCCAGGACAGCCAGGGCTACACAGAGAAAAAAAAAATTGTCTTTCAAAATTTCCCTTCTGCTCAGTACCAAT
CTCAATGGCTAAAATTGCTTCTCTTCTGAGGGAAACTTGGACTTCCAAGAGAACAGCCCAAACCTAGTGATTTTTAAGATCCAGGTGGAACTGCTTCC
ATAGTAATTTACTTCCTTTCGGCTTCTGAGCTCTGTGATTGTAGAGTGTGTGTGTGTGTGTGTGTGCGTGTGTGTGTGTGTGATACATAAGATTGAAC
CAGGCCTTGCACATGCTGTGAAAGCCCTCTACTGCCCCGCTGGGCCTCCACTCCTGGGTTCATCTTAAAAAGTAATCATCAGGGTGGGCTAGTGAGGT
TGCTTAGCCTGCTTACTTAAGTCTGCTCCCCAGGAAGAAGAAAACCAACTTACAAAAGTCATCCTCTGACCTTCACACAACATTGTGGAACAAACAAA
ATAAACAAGTAAATGCAATAAACCTTTTAAAATAATAATAATCCTGAGAGGTTTTCTTAGACCTGCTTAAAGTCACCTCTTCAGCTGTTGCCAGATTC
TTTGCTTTCAGTTTCTAACCCCAGATGAACCAACCCATTGTGAACATCTCTGGAAGGTCCTCAGGACGTGAGCGCTGAGATCTGTCCTTGTCCCTCTG
TGTCAGCACGTCAAGCATGGCAGCTGCTTTCCCAGCATTCTTCTGTTTTCACTGTCAGTGTGTCTGAAAAAAAATTCTCAGGTGTTGGAACGGGCTCC
TTGTCCACTGAACCTTGCTAGGCGGTCAGATGAGTGAAGGCCTCTGCTCCTACAGTTAATCAGGAAACTCCTTCCCAGTGTCAGGTCATTAGGCTGAG
CCCTGGAGCCTTGACTAGCATAGTTTGGAACCCAAAATTAGGGATTCATATTTAATTGCCCCTTAGATTTTTTTTTTTTTTTTTTTGAGATAAGAGTC
TGATTTTGTAGCCCTCACTGGCCTGGAATTCTCTCTATAGACCAGGCTGGCCTTGAACTCACAGAGCTCCAACTATCTGCCCTGTGAGTACCCGGATT
ACACAACCACACCCGGCTTTGTTTGTAGTTTTTGAGTCAGGGTCTCATATAGTCCAGGCCAGCCCTACACTTATGAAGCTGAGGCTGTCTTTGACCTC
CTGATTTTCTCCTTTCCATCCTAAGTGTGGGCACCACTCCTTGTTGGGCTTTTTAACTTTTAGTAGGTGATGAGGGTTGTCTTATGTGTCTGTCACCT
TTGACCCAGTCTGGTTGTAGGTGCCATGGTCCAGTGTTCACTGATGGCTTTCTAATGACTGCTGGAGTCTGGGTACCCTTGATCAAAGCTTGGAAAGG
GTAGATTTGTTAGCCCTCTTTGGTGCCCTGTGGGATGTGGAGGTCTAGCACTAAAAACTAAAAGCAAGTATCTCAGACAATAATATAACAGGTTGAGA
AGTTCAGGGGAAGCAGAAAAAAACAACAGTATAACTTTTTCTTTTTAAAAAACTGATTTTCATGAGGAACATGAAAGGTTAGCTTGCCTATCTGTGGG
GTTTTTTGGGGGGGGAGTGTGTCAGGGCTATCTGAACTTTGGAACTAGAGTTAGATGTGGTTGTGAGACCCCATATGAGTGCTGGGATCGAACCTCAT
ACTGGTTACGCTGAGTAACGAGTGCTCTGAACTGCCAAGCCATCTCTCTAGCCCCATCTGGTTTTTGTGGGGTTTTATTAAGAAATACTTTTGCTAAC
ATTTTACAGTTTGTCCATGACAGATCAGAGAGTGTTTTGGAACCTTCCTGCGTGGACTGCTTTGTTCATTAGAGAGAAGAGGTGCGATGGTGACGGCC
CACAGTCCCAGCACTGGGGAGGTGGAGGCAGGAAGGTCAGTATGTTTAAGGTCATCCTTAGCCAGGGAGATGGCTCAGAAGGGAGAAATACTTGTCAC
ACAAGCTTGATGACCCAAGTGTGACCCATGGAGCTGACTCAGGAAAGATGTCCTCTGGTTTCCACACAGGAGCCATAGCATATGCATGCTTGCACTCA
CGAACACTAAGAGTGATCATAATTAAGGGCTAGTGAGATGGCTCGGCAGGGGGAGAACTTGTCTTCATGTGGGGATGATCCCAAATCCTGGAACTCAC
TCTGTAGACCAGGCTGGCCTCGAACTCAGAAAATCCACCTGCCTCTGCCTCCCGGGTGTGCCACCACGCCCGGCAAGAGAAAGGGTCTTAACTGTCAG
GAGAAAACCTGAAAGAGGGGAAGGAAACTCCCGAGGAAGGTCGTCTTTATTGTCATTTCTTTGTAGCTTATGCTTTCTCTCCCCCCAAGCCCAGTGGT
AGAAATCATCCTTTCTTTAGAAACCTCACCTATGAAAGTCACAGTAGATCGTATTGTGACACAGTCCACTGGTGGAGGCCCCTCACTGCTAAGGACCC
ATAAGAAGGCAGAAGTTCTGAAAGTCCCTAGGAACCAGAAATAGTTCAGACTTAGTCTTAGGAATGTGTTGAAATAACTACTGTTTCTTCTCTTTAAA
CTTCATTAGAAGTTTTTCACATCTTTTCAGATTTAAAAATCTGAACACATTTTCCTCCTGGTTCCTCCATTGCATTTGCCCTAGACCAGCTATTTCTG
CTTAGGGCCATAACTCCTTTTTTAAAGTAGTTGGTTTTTTTGTTTTTTTTGTTTTTGTTTTTTGGTGTTTTGTTTTGTTTTAATGTGCATTGGTGTTT
TGCTTGCATGTATATCTGTGCCAGGGTGCCATGATCATCTGGAATTGGTGTTATGGGCAACTGTGAGGTGCCATGTGGATGCTATGAATTGAGCTAGG
TCTTCTGGAAAAGCAGCCAGTCATCTTAACTACTGAGCCATCTCTCCAGCGCCAAGAACCCATTTCTTTCTTTCTTTCTTTCTTTCTTTCTTTCTTTC
TTTCTTTCTTTCTTTCTTTTTTTCTCTCTCTCTCTCTTTCTTTCCTTCCTTCCGTCTGCCCGCCTTTCTTTCTTTCTTTCTTTCTTTCTTTCTTTCTT
TCTTTCTTTCTCTTTCCTTCTCTCTCTCTTTCTATGGTTTTAGAGCTTTATTGTAGAAAGGCAGAGAGAAAGACGGTAGAAAGAAAAAGAGAGGCCAG
CCATGGCCACGTGGAGAGAAGGGGAAAGGGAGGAAGGTAGGGCTAGAGATGAGAATAAGAAAGGTGAGAGAGCTAAAGAACCCAAGTGATTAGAATCA
GACTCCCTAATGTGTGCATCCATGGTATGCATGTGCACGGGTGGGCTGCTTCATAGTCTGAAATTATGCAAACAGCTTCCAGTTTTCTCTGAAATCTC
AAGGTGACACCTCATTTGTCACTAGACATTGATGATAAAAGTATACATGGCTTGTCTCATGATGTGGCTCGTTGCCTAGGAACAGGGGTGTCAGACTG
TCCAGGAATGTCAGCATTGCTCCTGCCTACCACTGTCTTTCATTGCATCATCTCTTCCTCCAGAGTAGCTTGCTAGCCAGTGAACAAGTTCCTCTCAC
TAGTGAACATTTGGTTTTCTACCCTGATAACTTAAGAATGGTTTCCTGTCGGGCATGGTGGCGCACGCCTTTAATCCCAGCACTCGGGAGGCAGAGGC
AGGCAGATTTCTTAGTTTGAGGCCAGCCTGGTCTATAGAGTGAGTTCCAGGACAGACAGGGCTACACAGTGAAACCCTGTCTCTAAAAACAACAACAA
CAAAAAAACCTTCAGTACAGGGGACAGAGAACCCCCTATTGGTTCAGTCAATAGAAAAATCATTTGCTATCATTAAGAGCGAAGGTAGGATTTCTGAA
AATGGAGTCGCTTCAAGACTTCTTCAGCCTTGGCATTATCCCTTAGTTCCTTTTCTCCGTGTGGTTTTCAAACCCAGAGTCTGAGACAGCTGGTGTAA
TATAAGAAAAATACTCAAGTCATGTGAGATGCAGCTGGGTGCCCCGCTTTATAGCCAATTATGCAAGAACTCTCTAGTCTACAAATGCGGAAGTAAAA
ATCCCTTCTCTCTGCCTTATGTAAATCTTCTAGCTGATCTCCCAAGCATTTTCTGTATCATATCTATAAATATCTGTAAAGTTCACACTCTAGAGAAA
CAAGCGCATGCTGGCCAATCTCACTGGCCACTTACTTATACTGGTTTAGGAAAATCCTGCCTCACATTCTTTTCTTCACATTAATGATCATTGGATTC
CTCTGCCCAACGTTTGTGCTGAAAATGATTTCCTAACCAGTAAAGACTTGCTGAGGTAGCACTCTAGAACCCCAGTGTTTAACCTAGCTGTTATGGTA
ACAGTGGAACTCAGGGGGTTTTGCTCTCAGTTTTCCCTACCCAGGCAGAAAAGAATTCCATAGCAGGAGTGGACCTAGAACTCAGGGTTGGGTATGAG
GCTCCAGTGTGCTAGATTGTGTGGTTTTACCTTATCTGACACCCGTCCTGTCTTTCCTGTAGCCTGTTAGATCAGTGTAAAGAGCAGCCAAGACAGCA
AGACACTGAAGAGGGTTTGGGTCACACAGAGGAATCTCTTTCAGTTCTTGCCATGATCAGAAGCTCATAGGGAAGCCGTAAGAAAGAGCAGATGAGTG
TAGAGAAGAAAGCCAGTTTGCCGAAGGACAGGAGCAGCGCTCTCATCTGGATGCCAGCAATGCGCTGTGCTAACAATAGGGCTCTGTGCCATTGGCAG
TTTGAGAGAGGGAAACTACATGACATTGGGACTTGGGAGCTGTCCTGCCTCTATCTAGCCAGCCAGAGTGAATGGGGTCAGTGTTAACGGCCTTCATC
TATGCAGTCACAAAGGTAAAGATGATACTGGGAGCATATTCCCTCCTGGGAGGCCTTGGCCTCACGCCTCAGCAACTGTGGCAAGACGTGCTAAATAA
CACCTTTGTTTGAGCCAGGGTTATGCATCGTCAACAATTTCATGCCAAGTCTAGGTGGTTTCAGATCAAGCAGCAGGGACGTGGACATGGACCATCCC
TCTGTGCAGCTCTGAAGTAGAGCCAAGCACACTGCCTGCCTGTAAGTCCCTGCCCACATTCCCTTTACCCCCTTTCACAGTTCTCCAAGTACCCTCCC
TGTCTTTCTGGTTTGGGGGGAGGGGGAGCGTATCAAAGTGAAGTGCACATGACTAGATGTAGGTAGCCAGAACCTATGGTTTGGGGTGATAAAGATGT
GATCTGGGCTGGGCACATGACGAAGCAAAAGAACCACTGCAAATTTGAGGGTTGCCTGGTGTCACAGTAAATGGCAGGCTAATCAGGGCTACATAATG
AAAGCCTGTGTTTAAAACAAGAGAAAAGGGAAATGTGTTTTTGTCTGTCCCGGAGCCTTTGCAAAGATGCCATCAGGAGCCCCTTATGTTCATTCCTC
CCAGCATCCTAAGCCAGAGCTCTTTTCCCCATCACCCGACCCTTCCTAGAGGATTGGCTCTCTGTGTCACTTCGTAGATGATGAAGCAGTCAGACATG
CCGATGTAGATGGTGGCTCCCTTGAATGATGGCCTATTCGCTCGTACTTAGAGGTTACCTGCGTTCTCCTGCTTTCTGCTATGTTAAAGACAATCCTA
GAAATGTTACCTCTCAACAGGAGACATTAGTCTTTGTGAATAAGTTTTATAAGAACTTTCTTACTATACACTAGAAGTCCTCTTAAGATTAAAAGAAG
CAAATGCTCTTATAAGTGAAGGATAACTTCATTGACTCCCTGTGTCTTTATTTCCTTGAATTCTTAGTAGAGGGACTGATCTTTAGGCTCTGACCCAG
TTAGTAATTCCAGCGGTGAGGGAAAGCCTAGTTTCTGATGTCTGGTCTGATTTCTGACAATGCCAGTAAGTCCCTTCACAAGAGAGGATCCCTCAGCC
TTGGTTAAAGCTCAGATGGCCTCTGATGAGTTTCTGCTTCCCAACCCTCGCCCGCCTGCCCACCCACCCTCCTGCAAACCCTGTTTCCTTTGCCTCTT
ACCTCTTCCTGAAATTATGCCCACCACGATCTCCTGCCCTAGACCCTCTGCGGCCTTTCCAGCAGAGGCCCCTCCTTGAAGGAGTAGAAGGATGATAA
CCCAGAAAACAGTCTTTACAAAGCACAAAAATACATTTTACGATAGTCATTTACTAAGTTTTCATATGATCTGGGAAAAGTCAGAAGCTCAAAGGGAG
TTGATGAAAAGCTGTACTCCTAAGCTTAGCAGTACCAGAACAGCCTTGGAGTCCTCTGCAAAGCAAACAGATCCCAGGGGGCTGGGAATTCACAAGAG
CAGATAAGCATGGCTGCACAGTTTGGGGGTGGAGACAGGGAAAGGCTGAGGCCCTGGACAGGGACTTGGAAAGATTATGGCAACAGAGGTGGAAAGGC
TAAATCCAGGTCTGCTGCATCCAAATAGCAACATTGACATCAAATACAGCCACAGTGTGCAATTTTAAGACCAGAAAAATGAGCCCGGATTTTTTAAC
TGCACAAAAGCCAAGTGAGCATTTTATGTTGTTACCCCAGTGACATCCTGAGTTAACAGTGGCCTTATCCCTTAGCTAAAGCCCCAGTGAGATGAGGT
AATGTTTGCCCTTATTTCAGAGTCGTGGGAAGCTGTTTCAGAGCCTAGGAAAGAAGGATGAGACACTGAGTCACACTGACCCCTGCTAGATTGAGATC
TAATCCCTACTTTGTTGTCTGCTCTGGGTAATTAGATGGCTTCACTCCTTCTTCTTCTTCTTCTTCTTCCTCCTCCTCTCCTCTTCCTCCTCTTCCTC
CTCCTCCTCCTCTTCCTCCTCTTCTGCTTCCTCCTCTTCACTTTTTAAAAGATCACAACTCTTAAGATTTTATTTGCAAAGCTTACTCTTGATAGTGC
ATTGGTCACTTAGAGGTAAATTGAGTCTTACTGCTAATACAGTGGAGACAGACAGTTTATCATAGAACTCAAGGAGGCCAGAAGACAGGCTAGCCTTT
GTTTGGATAGCAGCACTCTATCATGGTACCCTCTGGCCGGCCAGTCAGCTCTCTGACTCCCTTATCTCAGTTTCCCTGAGCTGGGATGGAAGGCTGAG
GCCGACCCAATTTTCCAGGCCCTGGTTCTTTGCCCTTGTCTGTGGCTGACTTGACTTCCATTCCCTCTGAGGGCATCACTCTGCAAAGTCTGCTTTCT
CGAGACCTGTCACCTACTTGCTCCTTCCATCCTTTTGTAAGAACCAAAGGAAGCCTGTCTAGGTTGGTCCTTCTCAGGCATTCCTGTCTGGCCATGGC
ATCTGTTTGTTTATCCACGAAAAGGGACGCATGGTGGATGTGATGTCAGTCAGTGTCCAGGCCTCCTGGAGGACAGACTGGATAAACTGGGCAGGAAC
TGATTCTTTTCACTGCCTTTGGGGTTTAGATGCCAGGAAACATGAGATGCCCATGATATGTGTGCTATGAAAATATTTGCTCAAAGCAGGCCTGAAAT
CTCCAGGCTCTTGTTCTTGTGGGGTTTTACATACCTGTCCTTGCTTCCAGGTCTGACATTGACCTGCCTTGACCTGCCCTGTGTTTTTTTATTGTTGT
TGCTTCCCTTAGAGTTCCTTCACTTTGCAGTGACTAACCCCCTCCCCCCCCCCCAAAAAAAAACCTCAGACTTCCAGATAGACCAGTGTGTAAGAGGG
CCCTTGGAGCCCCAAAGTGGGTCATTTCACTTCCCTCCCTTGCTTGCCCCTGTTCTCATGAGTGATTGGCATAGGTGGGCCCATTGATAGAGCTTAGT
TGGCAGTACTGCCAGACCTGGAATCAGGAGATGGCCCCAGGGATGAAACAGAGTCGAGATCTGTGCTAGTATATCTATGGTGACTTGGCTTCAGCTCC
TCACTGCTGGGTTGGTTACGGACTCTAGAAGAGTCCGTGTGTTCTTGGCTACTCCCTTCTGATCTGTAGAAAGAGAACACTCTTCTAAGAGCACAAAC
CAAAAAGGACATGAAAGCCTACCTCAAGAATGTACTGAAGTTTGTCCTAGGGAAGGAACTAGCCGGTGCCTTTCTACCGAACAGGAAGGTCAGGCCTG
CAGCAGAGATGTCAGTGTCTCATCCAAATGCTAGACTGTCCTGGACACTAATTGTGATTGAGACTAATTGGTAGTCACTTGACTAATGGTACTTTGAG
TTGGTGGCGGAGGACAAGAGGACTATGGGGCTGAGGGGTGGGGGAATGTGCATAGGACAGAATTGGCTGCCTGGGGCTGGGTGGGGCTCCTGGTGACT
GGGATCAGATAGCTAGAAAAAACAGAGCAGTTACGTAACTGTGTTTACATGGGCTCAGGTGGCTGTGGCAACCATGGCAGTCCTTGACCCAAGGCCAT
GCTGGAGGGGTGCAGGCAGCCGACTACTAGGGAAGGAGGTCCCTCTTGAGCAGAAGGGTGTGCAGGAGACAGAGCATGGCATACTGACCAGGGCTGCA
CTATCTGGGACAATGTTACCTGGCTCTAATGTGCCACTGGAAGCTACTCAGGGTGAGGCAGGCAGGCAGCAGCCAGCTACAGCTAGGGGGAGGGGTCA
GGAAACCAGACACCTGGCTCTCTAAGTCCAGTCTCTGAATTCTTGCTACATTTTTCTCCCTAATCTTCTTAAAAGAGGAAGAATTCTCCCTAAATCTG
GGCAGAAAAGAGGGCAGGAGATTATTTAAGTTTCCTTGGACTTACCTGCCCTGCGGTCCTAGCGCCTCGTGCCTCCGTGTTTTGTAAAGCCATCCGAA
TACTTGTGAACACAGTGAGCTCCAACCTGAGAGGTGGCGAGCCAGCCCCGGCCTGACTGACAAAGCAGAGCTGCAGCAGTTCAGCCCTCCATTGCCAC
CCTCCCCGTTCCTCCCAACCTACATCCTCCAAGACTGTTTACTAGACTCCCCATCCCCATAGAGATGGGACTGGTAGCCAGTCCTCAGCTCCCTTGCC
CACACTGTGCCTTCTCTTGTGAGAA
MOUSE SEQUENCE - mRNA
GTTGTGTCCCTCAGCCGCGGTCGCCGTAGCTCTCGACGCGGTCGTCCCCGCCGAGCCGCGGCGCCGCCGCAGGGAGGGAGCGGCGCTGCGGCCCGCCG
CGTCCGAGGACGCCCCGTATTCCGGGGCCGGCACGTGGCTGCCGCTCGCCGAGCGCAGCCCGCCTAGGAGGAAGGCGGCGGCTTGCGTCCAGCGGGCC
GCCGGAGCCGGGAGGAGACCATGTCCGTGAGGCGCCCTCAGTTCTCCACGACCGAGCGCGTCATCAAAGCTGTCCCCTTTCCTCCAACCCGACGGCTA
ACTTTGAAGGAAGTTTTTGAGAATGGGAAACCTAAAATGGATCTTTTGAAAAACCATTTAGTAAAAGAAGGTCGGGTGGAAGAGGAGGTGGCCTTAAA
GATCATCAATGATGGGGCTGCCATCCTGAAGCAGGAGAAGACCATGATAGAGGTGGAGGCTCCGATCACAGTGTGTGGTGATGTTCATGGACAATTCT
TTGACCTGATGAAGTTGTTTGAAGTTGGGGGATCACCTAGTAATACTCGCTACCTCTTCCTGGGTGACTATGTGGACAGAGGCTATTTCAGTATAGAG
TGTGTGCTGTATCTATGGAGCTTAAAGATTAACCATCCTAAAACATTGTTTCTGCTTCGAGGAAATCATGAATGCAGGCATCTTACAGAGTACTTCAC
CTTCAAACAGGAATGTCGGATCAAGTATTCAGAGATGGTGTACGATGCGTGCATGCACACTTTCGACTGTCTTCCTCTTGCTGCCCTCTTAAACCAGC
AGTTTCTCTGTGTACATGGAGGAATGTCTCCTGAAATTACTTGTTTAGAGGACATTAGGAAATTAGATAGGTTTTCTGAGCCTCCTGCTTTTGGGCCA
GTGTGTGACCTGCTGTGGTCTGATCCCTTAGAGGACTACGGCAGCGAGAAGACCCTGGAGCACTATACCCACAACACTGTCCGAGGCTGCTCCTACTT
CTTCAGTTACCCTGCAGTTTGTGAATTTTTACAGAACAACAGTTTATTATCAATAATCAGAGCCCATGAAGCCCAGGATGCGGGGTACCGAATGTATA
GGAAGAACCAAGCAACTGGCTTTCCGTCACTTATTACGATTTTCTCTGCCCCTAATTACCTAGATGTCTATAACAATAAAGCTGCAGTGTTGAAGTAT
GAAAACAATGTCATGAACATCAGGCAGTTCAACTGTTCCCCACACCCCTACTGGCTCCCAAACTTCATCCATGTTTTCACGTGGTCTTTGCCTTTTGT
TGGAGAGAAAGTGACAGAGATGCTGGTCAATATTCTCAACATATGCTCGGATGAAGAAATGAACGTAACCGATGAAGAAGGAGCTACTACAGGTCGAA
AAGAAGTCATCAAGAATAAAATCCGAGCCATTGGGAAAATGGCCCGGGTCTTTACGGTTCTTCGGGAAGAGAGTGAGAATGTGCTGACCCTCAAGGGC
CTCACTCCCACAGGCACACTCCCACTGGGGGTCCTCTCTGGAGGAAAGCAGACCATTGAGACTGCCAAACAAGAAGCCGCAGAGGAGCGGGAAGCCAT
CAGAGGTTTTACAATTGCACACAGGATCCGAAGTTTTGAAGAAGCCCGAGGTCTAGACCGAATTAATGAGAGAATGCCACCCCGAAAAGAGGCTTCAT
ATCATCATGATGCAGGGAGGATGCACTCACACTCGCATCCGCCACACCCACAGGCGTCAAGAAGGACCGACCATGGAAAGAAAGCCCTGTAATGACTC
AGGGCCCTGTGCAGCACAGATGGGTCCCAACCCTATGAGCAAATCACATTTATTTATACTGGAAATGAAACAGGAACAACTCAAACAACTTAAACTTG
GAGGTGCATTTGTAATTCAGTCGCATTTATTCTGTAAGAAAAATGACCATTTTATAAATTCTTCTAATTTATGTTCAATATAAAAAAAAAAAAAAAAA
AAAA
MOUSE SEQUENCE - CODING
ATGTCCGTGAGGCGCCCTCAGTTCTCCACGACCGAGCGCGTCATCAAAGCTGTCCCCTTTCCTCCAACCCGACGGCTAACTTTGAAGGAAGTTTTTGA
GAATGGGAAACCTAAAATGGATCTTTTGAAAAACCATTTAGTAAAAGAAGGTCGGGTGGAAGAGGAGGTGGCCTTAAAGATCATCAATGATGGGGCTG
CCATCCTGAAGCAGGAGAAGACCATGATAGAGGTGGAGGCTCCGATCACAGTGTGTGGTGATGTTCATGGACAATTCTTTGACCTGATGAAGTTGTTT
GAAGTTGGGGGATCACCTAGTAATACTCGCTACCTCTTCCTGGGTGACTATGTGGACAGAGGCTATTTCAGTATAGAGTGTGTGCTGTATCTATGGAG
CTTAAAGATTAACCATCCTAAAACATTGTTTCTGCTTCGAGGAAATCATGAATGCAGGCATCTTACAGAGTACTTCACCTTCAAACAGGAATGTCGGA
TCAAGTATTCAGAGATGGTGTACGATGCGTGCATGCACACTTTCGACTGTCTTCCTCTTGCTGCCCTCTTAAACCAGCAGTTTCTCTGTGTACATGGA
GGAATGTCTCCTGAAATTACTTGTTTAGAGGACATTAGGAAATTAGATAGGTTTTCTGAGCCTCCTGCTTTTGGGCCAGTGTGTGACCTGCTGTGGTC
TGATCCCTTAGAGGACTACGGCAGCGAGAAGACCCTGGAGCACTATACCCACAACACTGTCCGAGGCTGCTCCTACTTCTTCAGTTACCCTGCAGTTT
GTGAATTTTTACAGAACAACAGTTTATTATCAATAATCAGAGCCCATGAAGCCCAGGATGCGGGGTACCGAATGTATAGGAAGAACCAAGCAACTGGC
TTTCCGTCACTTATTACGATTTTCTCTGCCCCTAATTACCTAGATGTCTATAACAATAAAGCTGCAGTGTTGAAGTATGAAAACAATGTCATGAACAT
CAGGCAGTTCAACTGTTCCCCACACCCCTACTGGCTCCCAAACTTCATGGATGTTTTCACGTGGTCTTTGCCTTTTGTTGGAGAGAAAGTGACAGAGA
TGCTGGTCAATATTCTCAACATATGCTCGGATGAAGAAATGAACGTAACCGATGAAGAAGGAGCTACTACAGGTCGAAAAGAAGTCATCAAGAATAAA
ATCCGAGCCATTGGGAAAATGGCCCGGGTCTTTACGGTTCTTCGGGAAGAGAGTGAGAATGTGCTGACCCTCAAGGGCCTCACTCCCACAGGCACACT
CCCACTGGGGGTCCTCTCTGGAGGAAAGCAGACCATTGAGACTGCCAAACAAGAAGCCGCAGAGGAGCGGGAAGCCATCAGAGGTTTTACAATTGCAC
ACAGGATCCGAAGTTTTGAAGAAGCCCGAGGTCTAGACCGAATTAATGAGAGAATGCCACCCCGAAAAGAGGCTTCATATCATCATGATGCAGGGAGG
ATGCACTCACACTCGCATCCGCCACACCCACAGGCGTCAAGAAGGACCGACCATGGAAAGAAAGCCCTGTAA
HUMAN SEQUENCE - GENOMIC
GGGACTACAGGCACGTGCCACCACACCTGGCTAATTTTTTGTATTTTTAGTAGAGACAGGGTTTCACTGTGTTAGCCAGGATAGTCTCGATCGCCTGA
CCTCATGATCCACCTGCCTCAGCCTCCCAAAGTGCTGGGATTACAGACGTGAGCCACCGTGCCTGGCCTATATAGTGCTTTTCATATAAAATTGCAGA
TTATACATTATAATATACCTTTTGTTTTGTGTAGCTATTTTAGAAATGCTCATTTTACTATGGTTTTTAAAATATTCTACAGTTCTCTTACCTGCATT
TAACACAGAAGTTGAACATTTAATGGCTTCATATAAATAGAAACTGACGCCAGGTTACTTTCACACAACAGTGGAAACAAGGGTTCCCTGTGCTTTTG
TTAAATTAAAAGTGTTCATAATTATGTCTCAGAGGAGCTGGTCATTAATCAAGGTTTAGGAAGCGCTTAGGGAGTTTCATGAGGAAATACACCAAAGC
TCACAAGCCCTGTAAGAAGGAAACCCAGATTTGAAGAAATGTGTAGAAATGATAGTTAAACATGAATCTTGGTTATTTCAAATATTTATGTCTTCTGA
CTGACCAATGCAGAAAATAAAAAAAAGTGGGGGGATACAAAATTCACCAAGTGGACATCTGGCCAGGTGGAGAGATCCACCAGCTGAATGAAATTGGG
CTCTCCAATGATTCTTGAGAATTGTTAATGTCTAAAAACACCACCACACAAGCAATGAACTTAAACCTAATTTGTTATGGAAAACAATTGTTTGGGAA
GGGAGCTCAATTAGAGCTTTCTTCTTTTTAAATTTTCATGGAGTGGCTCTAGATGTTTTGTTTGTTTTTGTTTTAATCAGAAAAGACATTGAGAAAGC
AATGAATGTTTAACTCTATTGGAACTAGGCCACTAAAAGAAAAGAGCAGCCGGCACGGTGCCTCATGCCTCTAATCCCAACACTTTGTGAGGCCAAGG
TGGGAGGATCGCTTTAGTCCAGGAGTTCCAGACCAGAGTGGGCAACATAGGAAGACCCCCCATCTCTAAAAAAAGAAAAAAATAGCTGGACATGGCAG
TGGCATGCCTGTGGTCCCAGCTACTTGGGAGGCTGAGGTTAGAGGATCACTTGAGCCCAGGGAGATAGAGGCTGCAGTGAGCTATGATCATGCCACTG
CACTCCAGCTTGGGCGACAGAGCAAGACCCTGTCTCAATAAATAAACATATAAATAAATAGGTGAAGATAAAGAAAAGTGCTTCCTTCTTTCTTTTTC
TTTCACTCTGTCTCTTTCTCTCTGCTCCTTCCCTCCCTTCTCTCTCTCTTCTCCCTCCTTCCTTTTTCTCTCTCTCTCTCTCTTTTTCTCTCTTTCTT
TCTTTTAGAACAGGGTCTTGCTCTGCCACCCAGGCTGTAGTGTGGTGGTGTGATCATGGTTCACTTCAGCCTCAACTCCCTGGGTCCAAGTGATCCTC
CTACCTCGGCCTCCCGAGTAGCTGGGACCACAGGTGTTCACCACCGTGCCCGGCTAATTTTTTTTTTTTTTTTTTTGAGATGGAGTCCTGCTCTTGTT
GCCCACGCTGGAGTGCAATGGCATGATCTCAGCTCACTGCAACCTCCGGCTCCCAGGTTCAAGTGATTCTCCTGTCTCAGCCTCCCAAGTAGCTGGGA
TTACAGGCACCTGCCACAACACCCGGCTATTTTTTTTTTTTTTTTTTTTGTAGAGATGGGTTTTACCATGTTGGACAGGTTGGTCTCGAACTCCTGAC
CTCAGGTGATCCACCCACCTCAGCCTCCCAAAGTGCTGGGATTATAGGCATTAACCATCGTGCTCGGCCACCTGGCTAATTTTTAAAAAAAATTTTTG
GTAAAGATGGGGTCTCCCTATGTCATTGCCCAGGCCGGTCTGGAACTCCTGGGCTCAAGCGATCCTTGTGCCTCAGCCTCCCAAAGTGCTGGGATTAC
AGTCATGAGCCTCTGCACCTGGCCTTATTTTCACCTATTTATAAAGCAATACTTTCTCACCACAGAAAATTGGAAAGATAAAGAAATTTTTTTTATTA
CACCACCAAGTGATGGCTACTGTTCACATTTTTTTCCTTGATAGATTATGCTCAGTGAGGGTATGAATTATGTCAGTTTTGGTTCGTTTCCATATCCT
TAACACAGTGCCTGGCACACAGTCCACACCTAATTCATACTTGTTGAATGAGTATATTAATATTTTCTTGAGGTCTTTTATTTTTAAAGAAATGGAGT
CTTGCTATGTTGTCCAGGATGGAGTGCAGTGGCTATTCACAGGCAAGAAAATAGTGTAATATGACCTCAAACTCCTGGGCTCAATAGATCCTCCTGCC
TCAGCCTCCCAAGCAGCTGGGACTACAGGTGAATACCACTATGCCTGGCTTAAGACTTTTATTTTATGCTTTTTTTTTTTTTTTTTTTTTGAGACAGG
GTCTCACTCTGTCACCAAGGCTGGAATGCAGTGATGCGATCTTGGCTCACTGCAGCCTTAACCTCAGAGACCGCCCACCTCAGCCTCCCAAGCAATCC
TCCCATCTCAGCCTCCCAAGTAGCTGGGACCACAGGCGCATGCCACCACACCTAGCTAATTTTTCTGTATTTTTTATAGAGACGCGGTTTCACCATAT
TGCCCAAGCTGGTCTCAACCTCCTGAGCTCAAGTGATCTTCCCGCCTTGGCCTCCAAAGTGCTGAGATTACAGGCGTGAGCCACTGCACTCGGCCATG
TTTATGTTTTTGTTTTTGTTTTTTTAGATGGAGTTTTGCTCTGTTGCCCAGGCTGGAGTGCAATGGTGCCATCTCAGCTCACTGCAACCTCTGCCGCT
TGGGTTGAAGCGATTATCCTGTCTAAGCCTCCCGAGTAACTGAGCCAGCCATGTTTGTTAATAAAAATGGGGACAATACTGTATATATATGGTTATAT
TTTAGCTTTTTCACTTTTCAGTATCTCCTGAGGCTTTTCTCATGTCATTAAATATTTTTCTAATTTAAAATATGGTATTTCCTAACTGCATGATGTTC
CATTCAATCATATTTCATGTAACCATTTTTCCTATATGGATGAAATCATATAGTAGATATCATTTTGTGGCTTGCTTCTTTTGCTCAGCGTAATGTAT
TTTAGAGGTTCATTCATGTTGTTGCCTGTAGCAGTAATTATTTCTCTTTTATTAGCTGAATAGTATTCCATTCTATGGAATTTTACAATTAACATCTA
AAAAAAAATAATGAACTGCTTATAGGAGGGAAGCTGGGTGCTCTTTTCCACAGCACACATACTAAAATAGGAATGATACAGAGAAGATTACCATGGCT
CCTGTGCAAGGATGACACACACATTTGTGAAGCATTAAAAGAAAAAAAAAGAAGGAAACTGAGGGCAGGGAAATAAGTGAGAATTTACTTTTCTTCTA
CATACTTTTGCAGTATTTAATTTTTTTCTTTTCTTTTTTTTTCTTTTTTTTTTTTTGATACTGAGTCTTACTCGCCCAGGGTGGAGTGCAGTGGTGCA
ATCTCGGCTCACCGCAACCTCCGCCTCCTGGGTTCAAGCAATTCTCCTGCCCCAGCCTCCTGAGTAGCTGGGACTACAGGCGTGCGCCACCACGCCTG
GCTAATTTTTTGTATTTTTAGCAGAGATGGGGGTCTCACCATGTTGGCCAGGCTGGCCTCGAACTCCTGACCTCAGGTGATCCACCCACCTCGGCTTC
CCAAAGTGCTGGGATTACAGGCATGAGCACCATGCCCAGCCTTTAATTTTGTTAAACCATGGGCATGTGTTACTTTTAAAAATATATATTTAAAGCAT
TTAAAAAATTCTGAGAGCAGAGTTGGGTTCAAGTTGTACTTTGAACTACCTGTGTGACAGTGGGTAAGTGGCTTGATGTCTCTGGACTCATTTCCCTC
ACCTGTAATATAATAATGTTGGTCCATATGAATCCTTGGTCTTTGGCCTCTTAGATACTCCAATTGGCCAGTTATCTTTTGAATCAAACTTTATCTAC
TGAGGCATACTAAATTATTACATATAAGGAATGGAGGTGATTAGTGATTCATTATATCCACAAACACCTACTGAGCAACCACAATATAAATGGGCCAT
GCTAGAAACTGGAAATATAGGCTGGGTGCAGTGGCTCATGCCTGTAAATCCCAGCACTTTAGGAGGCAGGCCGATGTGGAAGGACCACTTGAGGCCAG
GAGTTCCAGACAGACTGGGCAACATAGCAAGGCCCCATCTCTGCAAAAAAGAAATTAAAAAAAGCTGGGCCTAGTGGTGTGTGTCTGTAGTCCCAGCT
ACTCAGGAGGCTGAAGTGGGAGGATTGCTAGAGGCTAGGAGTTGGAGGCTGCAGTGAGCTATGACGGTGCCACTGCACTCTAGCCTGGGCAACAGAGA
AGATCCTGTCTCAAAATACATAAATAAATGACAATTTTTAAAAAAGAAGAAACTGGATATAGTCTCTGCCTTAATAGTCTAGGTGGGGAAAACAGCCA
CATGTGAATAAATAATATCCTCCATGCATTAGGTGGTTGATCTGTTTACAAAACGCTCTTGGTCCACAGAGGAAGGCTTTACCTCTACCTGGATGGAT
TAAGAAAGGCTTCAGAGAGGTGAGTATAACAGGGCTGGATCTTAAGGGCATGGCTGAACTTGTGAGTCGAAGTAGAGAAGGAAGGGTGTTTCCAGGAT
ATAAGGATCAAATATCCAAAGGAGTGGAGCCCTGAACATATCCACTATATTCCAACCCCATGCCTCTGTACAGCTTTGCCCAACCTAGGACACCTCCC
TTCCTCCTTGCTGTTCACATCCCATCCTTCCTTAGAGGGCTGCAGCCCTGAAAAAACATAGCAAATGGTTTCAAGTGGCCGGCCTGAAAGATCTCATG
GGGCTACGGACAGAGAGAAGCTGTAGAGGTGAGTCAGGGTCAGGCTGGGAAGGAACTGTTGGGCCAGGTTAAAGGGTTCAAACTCGATCCCAAGGGCG
CCAGGGAGCCACTGAAGGGAGATGAGGAGCAGAGTCAATAGTCAGATTTTCTGTACAAAGTTGACTCTGGGCCAGGTATGGTGGCTCACACCTGTAGT
CCCAGTACTTTGGGGGGCTGAGGCAGGAGCATTGCTTGAGGCCAGGAGTTGGAGCCCAGCCTGGGCAACATTGCAAGACCGAGTCTCTACAAAAAATT
TAAAAAATTAGCTGAGTGTGGTGGTGTACACCTGCAGTCTCAGCTACTCAGGAGGCTAAGGTGGAAGGATTGCTTGAGCCTGGGAGGTTGAGGCTGCA
GTGAGCTGTAATCATGCCACTCCAGCCTGGGGGACACTCCAGCCTGGGGGACAGAGTGAGACCTTATCTCAAAAAAAAAAAAAGTTGATTCTGGGGTC
AATGTGGACAATGGATGAAGATAAGAGGTCAAGACCAAAGACTCAGGGACCAGTTAAGAAGCTGATGCAGCGGAGGGTTAGCCCCTATTCACTCCCAG
CCTTTATGCCCTGAGAATAGCTGATTCTATTACAGAATCAGTTGAGATGGATTCCCGCAGAGAGCAAGACACGTTCCCTGTGTCTGCCCCAAACAACC
CAAAGTCTTTTGAAACCTCTACTAGGATGGGAAAGCACCAATGCGATGGGTGAAGGTTTCTGGTGGTCTCAGCTTATACAGATCAAAGCCTTTCCAAC
CTGGTTTCCTCCCGGAGAAATCCATGGATCTAACGCCCTCTTCCAAACACAGAGATAGAAAGTGAGGAGAACTATTAGGAGCAGGGCTGCAATCCCAG
CTATCGGGAGGCTGAGGCAGGGAGAATTGCTTGAACCGGGGAGGCGGAGGTTGCAGTGAACCTAGATTGTGCCACTGCACTCCAGCCTGGGCAGCCTG
GGCGACAGAGTGAGACTCTGTCTCAATTAAAAGAAAAAAAAAAAAGAAAAGAAAAGAAAAAGAAAAGAAAACATTGCAAACCAATGGGAAGGGATGTA
TTATTCAATAATAGTAATGGAAAAATTAACCGACTACTGTATTTAGATCTCAAGTTGGGGCCATCTCATAACATCCAAATAAAATGAACCTCATTTAA
TGTGTTCCTATAGTTCAGTCTGTTCTCACAGCAAGCTCTGGAGCCTGAGTTGTACCACAAAGATATTCTTACCTTGAGGCAAACTTAGTCTGACCTGG
AGGCCCTGATAGCCATTGCCCCTAGGACTGGGGGAGGGATGGTCATAACTCCTTAGGGGAAGGGGCGCCCATTCAACCTAAGGCAATTCTCTGGAGAA
GGGGCAGCAATGAACTGTCAGCCACCAGCCCTCAAGAAACAGGCAGAGTACGGTAGCTCATGCCTATAATCCCAGTACTTTGAGAGGCCGATGCAGGA
GGATCACTTGAGCCCAGCAGTTCAAGGCTTCAGTGAGCTATGATGGCACCACTTCACTCCAGCCTGGGCAACAGAGCAAGACCTCATCTCTCTCTTAG
AACAAAAACAAAAACAAAAACAAAAACAGCAGCAACCAGAAGATGGTTATCCCTCATGGTGAAGGGGATCTGGGCAGGGCATCCCCGCCTCTGCTACA
GGGAGGGCCTATTAATATTATCATCCCCATATTACAAAAGAGGAAAGCAAGGCCTGAGAAGTGAAGGAACTTACCCATGCCCACAGCTGGAAGTGATG
GTATCAAGGTCTGTTGGGTGTCACTGGCTGTTGCTGGAAGACTAAGCAGCTATTGAAACAGAGGACCAGCAAGGAACTTGGAGAAAGGCAGAGCTACT
GAGTGAATCTCCTTAGAAAGGGGTGGGGAAGGCCGAGGCGGGCAGATCACGAGGTCAGGAAATCGAGACCACCCTGGCCAACATGGTGAAACCCCATC
TCTACTAAAAATACTAAAAATACAAAAAAATTAGCTGGGTGTGGTGGCATGCGACTGTAGTCCCAGCTACTTGGGAGGCTGAGGAAGGAGAATCACTT
GAACCCAGGAGGTGGAGTTTGCAGTGAGCTGAGATCACGCCACTGCTCTCCAGCCTGGTGACAGAGCGAGACTCCGTCTCAAAAAAAAAAAAAAAAAA
AAGTGGGGGTGGGGACACGAAGGGACAGGGCTGCAAGTTGCTCACAATTACTTTGCTAAAGCCAAGCTACAGAATGACATCTGGATTCTGTAAATTCA
TGTATACCATGCTTGAGGACTCATGCCGCATATTCCACGTGGTGCTATAGAAAAAGTTCGTGGTGCTATAGAAAAAGTTCAGGTACTGCACGTCTCTT
GACGATGATGGTGTTCACTAAGCCTCCTACCAGCTCTGTGACTTTGGGCAAGTTTCTTCACATCTCTGTGCCTCAGTTTCCTTATCTGTAAAATGGGG
ATAATCATATAAATGGGGTTGGTGTGAGGATTGAACGAGTTAGTATTTGTGGGGTTTTTTGGTTTTGTGTTTGAGATAGATGGAGTCTGTCTCTGTGC
CCAGGCTGGAATGCGGTGGCACCATCTTGGCTCTCTGCAACCTCCGCCTCCCGGGTTCGAGCGATTCTCCTGCCTCAGCGTCCCGAGTAGCTGGGACT
ACAGGCGTCCACCACCACGCCCAGCTAATTTTTGTATTTTTAGCAGAGATGAGTTTTTGCCACGTTGGCCAGGCTGGTCTCAAACTCGTGACCTCAGG
TGATTCGCCCACCTCAGCCTTCCAAAGTGCTGGGATTACAGGCATTAGCCACTGTGCCCGGCCTGCAACGTTTTCTTTTTGTTTTTTTCTTTTTGGAG
ACAGTCTCGCTCTGTCACCCAGGCTGGAGTGCCGGGGCGTAATCTTGGCTCACTGGAACCTCCGCCTCCCGGATTCAAGCGATTCTCCTCCCTCAGCC
TCTCGAGTAGCTGGGATTACAGGCATGTGCCACCACGCTAGGCTAATTTTTGTATTTTTAGTAGAGACGGGGTTTCACCACGTTGGCCAGGCTGTTTT
CAAACCCCTGACTTCAGGTGATCCGCCTGCCTTGGCCTCCCAAAGTGCTGGGATTACAGGCGTGAGCCACCTTGCCCGGCCCGCAATGTTTTTTCTTT
CTTTCCTTCTTTCTTTCTTTATTTTATTTTTTTTTTGAGACGGAGTCTCGCTCTATTGCCCAGGCTGGAGTGGTGCAGTGGCGCGATCTCGGCTCACT
GCAAGCTCCGCCTCCCGGGTTCACGCCATTCTCCTGCCTTAGTCTTCCAAGTAGCTAGGACTACAGGCGCCTGCCACCACGCCCGGCTAATTTTTTGT
ATTTTTAGTAGAGACGGGGTGGCACCGTGTTAGCCAGGATGGCCTCGATCTCCTGACCTCGTGATCCACCCACCTCGGCCTCCCAAAGTGCTGGGATT
ACAGGCGTGAGCCACCCCACCCGGTCTTTTTTTTTTTCTTTTTTGCCGGCCCGCAATGTTTTCTTAAACTTTTTATTTTTAGCTATCCTGTTGAGGTT
CTTCCAGATGAATTTTAGCCTCTGACCCAGGGGTATGATTTGATAGGCGTGGAGTTCTGGTTACTTCTTAAATGAAGAGTTTCCCCAGGATATCTGAA
CAATTCTCTTGGGTGGAAGACTCAAGGATCATGAAAACGAAAACCAAATTCTAGTCGGCTCCTAAAGTCCTTTTGCCTCCTGCGGTTACCATCAAAGA
GCTATGGTCAAAATTCCAAGGGATAATTCAGGGTCTGATTCCACTATATATCCCCAGACCGCCTATACATAAATCCATATTATTGGAGATTTACGCTT
AATCCTATAATCTTTTACCTAACAGCTGTCCAGTAGAATTTTCTGGATGTCCAATAAGGTAGCCGCGTGTAGCTCTTGAACACTTGAAATGTGGCTAG
TCCCCTAGAGGAACGGGATTAATTTAAATTTACATAGCTACAACTACTTATCGGTCACTGCGGTAATTGTTAAGACTGGTTTTGCCTGTAACTTGCAT
ATGTTTTATTGGACATCAGTTTCAATATTTCATTTGCATTGCTAAAAGTCAAAATCCTTTTGACTTTGCCAGTTACTCAGTGAGAGGACAGTTGGAGC
CAGTTTCTGTTCTAGGCTTACAGTTTTAAGGACAGATAAAGGTTAGTTTTTAACCTCGAGATGAACAGACTGTTAAGTTTCTACGGACTTGCCCATGG
GGTTCCACAGTGAGCCTGGGCCGGGGAGGCAGGTACTGTGTCAGGGGGAAGAAAAAAGGGGCTTCTAGCTGGGCAGGTGACACACAGGGCATAGGAAG
AGGTCAGTTCTTGTGGCCGAGGAGGTCCGGCCCGGGGGTCCCAGGAGCAGAGATCTCCCTTCTCTTCGATGTGGAAAGTGAGGAGGGAGCAGAGCCTT
GCTCAGAAACGGAGCTCCCCCAATCCCCCACCGCCCTAGCTACTGGACTGGAACTAGGATGGACACGAATGTCCTTCTCATTGTACTAACTGCACTCA
ACAAGCGGGAAAGATGAAGCGAGGGGGTTTAAATGTGTGCGTTTGTTGAATGACCCACAAAAACTGAAGGAGCGCGGCCGGACTGATCACACTCCTTG
AGACAAAGCGGGTGGGAGACCCAGAGGTGAGGAGGGTGGTCGCCTGTGGGCGAGGACTGGGAGGCCAGGGGTTCTCGGCGAGGCGGTCCCAGGAGGCG
GCGCGGAGCCGCTGCGAGGCCGCAGGGCCGGGTCGCAAGATGGCCGGGCCGGCCTCCCGCGCGGGTCGCGCGGGCCACGCGGGCGGGTTTCAGATGTT
CCGGGCCCGGGTAGGGGCTGGCAGGAGAGAAGGGGCCGGCTGCGGGGAGGGCTGGCTGAGAAGAAGCGAAAATGGGCGGTTAGCAGCAGGGACCCGGA
GCCGGAGGAGCCGAGAGCAGCGCGTGCGCCGAGCTCTACTGCCTCGCGGGAAGGCGGAAGGGTGGGGAGGGCGGCGCTCGGGGCGGGAGGCCCGGCCG
GGTCCGCTAGGACAGCGGGGCCGCTGGGAAGTTGTGAGAGCGGCGCTCGGGGGCGCGCTTGCGTGCACGAGGGCCCGGGCCGCGAGCAGCCGCGGCCG
TCCCGGTCGCCACCCTTAGCAGCGGTCGCGGTCGGTGCCGAAGCGGTGTTCCCCGCCTTAGCCGCTGGCGCCTCCCAAGAGAGCGGCCGGTGGGCCCT
CGTCCTGTCAGTGGCGTCGGAGGCCGGCGCTGCGGTGGCCGCGCCCTTCTGGTGCTCGGACACCGCTGAGGAGCCGGGGCCGGGCACGGCTGGCTGAC
GGCTCCGGGCAGCTAAGGCTGCCCGAGGAGAAGGCGGCGGCCGCGGCGTAGGCGCACGTCCGGCGGGCTCCTGGAGCCTGGAGGAGGCCGAGGGGACC
ATGTCCGGGAGGCGCTTCCACCTCTCCACCACCGACCGCGTCATCAAAGGTGCCTGGCGGGCCGGGCCTTCCTCTGGGACCCGCGGGAAACGGCCTTC
GGCTGGGCGGCGGCTCGGGGCGGAGGGAGGCTGGGGCCGGGCTGCGCCCACCCTAGGAGGGCTCGGAGGGGTGTAGACAGAGCCGGGCGGCAGCCTCC
GAGAGCAGCCACCCGGACCCGGCGTTTTCTGCTGCACCTGGTCAGGTGCCTGGCCGTCAGTGCCAAGTTCCCTCCCGAAGGGTTCAGGGTGCAGTGGG
GACTTCTTCTCCCACCGCCCCGAAGGGCCCGGATCCCTGTCTTTTTTTCTCTTTAAGAAACGACTCGGGGGAAGCCATCGGGGGTGGTGTGAGCAGGG
AGTCGACTCTTCCAAGTAAGATATTTAAGATTAGATCTTTCTTGACGTCCGCCCCCCACCCTTTTTTAGGACGTATACCATGTGCCTTTTGACCCGCT
TTCTCTAGATCATTGCAAGTCACTGCTATTACTGGAATAAACTTAGTATGAACAAGTCAGATAAATCCATTATATGATGTTTAAAATAAGTTTACGGA
GCTCTTGACAGGGTAATGGTATTTTATTTTCCCTTTGGTTATAAAGACTTGGAGAATGACGTTTTCTGATTCATATGTGTACCTGCGTTAGATTTCTG
TTGTTGTTATAAGAACAAAATGTAAAGGGCTCTTTGCTCTCTAAATCTTGTAGAGTTTTAGGTTATAGTTGTTTCTCCCTTTAAACTAAGAAGATGGA
TTACACATAACCACTAATTTATGTGCTGCTTTTATTCAGCCGTAAGTCTTAGGCACAACCCTTCTCTCTGAGATTAACAATCGGTTTGAAAGCGCTAT
TTTGTAAGAAGGGAATAGACTTAGACCAGCTGTGTTAGTGGCCTCTGGATGCAGGGGTTAGCGTGACAGTGGGGAAAATTATTAAGATACTTTTCTTC
TTTTTCATTTTGGTATTAGAAGGAGAAAGGCAACTAATACTAGTGACGGGAATACATTTTTAAAAGGTGAAACGCCTTATTGCATGTTCAGGGAGGGG
GAAGAATTTTAGTGTATAACTTAGGTTTCCCCCCTCTCTCCCCAAATCAGCCTCATAAATGTTAATTATCTGTACTACATAATTATAATGGGATTTTT
GAGATTTATCTGCTTAGCATTTGTATGCTATCGTTAGAGGTTTAACTGAATTGTTCATCTAACTGACATTATCCCCTGAACAGCTAAATTATTGTGAC
CATTTAAATGTGTCTTCATATTATTAGTGTATAGCCTATAAGAAGTATATAGCCCTCTCTTAAGATTCAGAGTGTACTTAACATAACCTTTTTTGAGG
AATCAAGGTCATTATGTACATTAATTTCTGTACTGTGCTGTTAATATGGCCGGTGCGTTCTGCTGTATAAAGTTGTTAGCAGTTTCGTCCTGTACTAA
AATGACTTCGCAATGCTATACCAGTTCAGTGTTTTGTTTGCTTTTAGTTTCTCTCTCCAGTACTGTTTTCTGTCAAAATATGCCCTTAACTGCTTTCT
AATCTGCTACGTCTTAGCTGAAGTAGGCAGGGAAACCAGATAAGCCAGAACTGTGGGTCAAGTCAGAATAAATAGGATCTTAGTGAAGCTTAGTGCAG
CCCTCAGGAATCTCACAATCAAATCACCATTGTGCCATGTAATTTCCACCATAACATGCCATAAGATCAAAAGAAGATTAAAAATCTTAAATGACCCT
TAAATATAGTAATAAATATTGTAAAATAAAATGTAATAAAATAAAAAAGTTAAAATGCAGGGGCTAGGGGCAGGCATGGTGGCTCACGCCTGTAACCC
CAGCACTTTGGAAGGCCCATATAGGCAGATCACTGAGGTCAGGAGTTCGAAACCAGCCTGGCCAACATGGCGAAACTCCATCTCTACTAAACATACAA
AAATTAGCTGGGCATGGTGGCGGGCACTGTAATCCCAGCTACTTGGGAGTCTGAGGCACGAGAATTGCTTGAACCCGGGAGGCGGAGGTTGCAGTGAG
CCAAAATGGTGCCACTGCATTCCAGTCTGGGCAACAGAGTGAAACTCTCTCTAAAAAAAAAAAAAAAAAAAAGCAGGGGCTGGTTTATTGCTACGCAT
GCTTATTATTAAATAAACACTGTCATCATATCCTGCAGGATGCAAATTTGTTCTTTACCAAAATTAATTACCGAATATTGTGTACTTTTAAGACTGCT
CTTAAATAAATGAAATAGAAATAATTTCAGGTCCTTAGAATGTATGGATTTCTCTCCTATATAAAAACTTCGGCATAAGAGCTTGACCACAGCATTTG
TGGCTTCATGATGACTTATGGTGCTTTTTTCAGGGTTTTCCAGAGTTTGAAATAGTTGAAGAAAATGAGGTGTCACGTGTGCATAGCTAAGGTCCATT
GTAACGGGTACTAAACCCTGGTCTGATGCTATCTTGTTTAGTCACAGCTGTCTGCATTTATTCACAGCTTGAAAGCTTTTTCAGAGGTGATACCACAC
CCCAGCTATTTTTGTTTCTGTCATTTCCTGTTAACTTTAAAGTGTACCTAATTGTTCTATTTGAGCAAATTTTGGAGAAGAGTGGGTATGTGGGAGAG
GTTTGTATTTTAGGTGTCCTTTAAATAGCAGCTTTCTTAAATTGAAGGATGTGGCTGGGCGTGGTGGCTCACACCTGTAATGCCAGCTACTTGGGAGG
CTGAGGCAGGAGAATCGCTTGAACCCAGGAGGCAGAGGCTATAGTGAGCCAAGATTGTGCCACTGTACTGTAGCCTGGGCGACAAGAGTGAAACTCAG
TCTCAAGCCAAAAAAAAAAAAAAAACTCATCTGTAAAATAGGGTTAATAATACCTTCCCTGTCAAGCCTGGGTCATGCAAGTGACATAAAGCATGTGA
AAAGGTCAAGTTAAGGTAGTACTTTGAGACATGATAATAGGTTGGTAATAGATCGCTTACTACAGAAGTAGTTAGTAGAGATATCATTAACATTTGAA
AATATCAGAAAGTATGTGAAATTATAAGCTACAAAACTGTTCCTAGTCAGGTGAATACCTATTCTACAGCATAATGGATATGTGGGTGAAATCTCGTC
GGTAACAATGTGAAGAGATCAGCACAGTTCTGTCTGGTACTCCCTAGGCAGCAGATTTCTTCTCTATCTGTTGAAGGTGTTTATTTTATGTTTAAACC
TTCTTATGATTTTACTCATTCATTAAATGTTTATTGAGTGCCATCTGCAATGTACCCTAGTAGGTTGCTCATTCATTTCTTCTTTGCTTTAAAAGTAG
CATTTCTGTGAGCCTTTAGAGCAGTTTATATGCACTGGGGCACCATAACTGGTATCTAAGCCAGAGAACTAGGCACCCAAATGATTAAATTGTGATAT
TCTGTCTGCTTTATTTTTTTTTCTAGTTTCCACGTTCCTTTTCCTGTTTAAGGACATGTTTTTTCTTTGACAGTGATTTTTATAAAAGCAGTTTATTT
CCCTGGAATGAATCTTTTCAGAAGCAACAAGGAAATTTCAGTAGGAATTACATGACAAATATAGAGAATCCATTGTCTTATTTTGTTACTTTATGGCC
AGATGGGGTTTAACAGAAAAGTCAGATTTCTTTTTAGTAAGGTGCTGATACATTTCTCCCTTTCTCTTAGAATTCAAATATATTGTGTATTCACAGAG
TACTGTACCATTTTTACCACACATGGCAGGCTACTGTTTCTTTAATATCGTGGCACTGGGAATATTGTGCTTATGGCTTTCTTAAAATATTTTAATAT
TAGTGTGGACAAAAATTGCTATTTCTACAAATATACCCTAAACCCGATTTCAATCTCTGGTACCTATACTTCATTTCTTTCCACAACTCTCCCCCGAC
TCTCCCTTTTTTATATCCATAATGGATTTACCGAGGGTTTAAAATTTTTTCTCTTAAATATAAGCTTAATCCTGCTACTTATACAATTTGAAAATCAG
AAAGGGAAGAAAAGAAAGACTTCATATTCCTTCCCAGCAGAAATAACTGCTTTTAATACCTTTGTGTGTTTCCATCTGCAGGTATATTTTGAATTCAA
AGGAAAATGGATCACACATATTAAAGCTTTTGTTATGCAAATATTTCATATTATTTTAACATAAATATTGTAATTTTACCTAAATATTTCCATAATGG
TTTCAATCAGCATGGAAATGGTTTTAGAGTGCTTATATTAATATTTTGGTTCTTTATATTTTCAGTGGGGACACATTAGTCACTTGATACAATACCAA
CCCAGAAGAAACATTTTCAAGTATAAATTTTCAATGTGAATACGTTTTAAAGTTGATATCTCCTGGATTTGTCAACAGTATTTGTTTAATTTATGCTT
CATTTACCATTGTGATTAACAAGCTGCCAAACTTTTGAAACATTCAAAATCAGTGCTAATCGAGATTCATACATCTGAGTAGAAATCTGGAACCTCTG
AAAGGGCATCTTAATTTAAACACTTAATTTTTTTCTTTTTGAGCAAGGATAAAGTATTCTTTCTTACCTATACGTTATTGGTTGTGATCAAAGCTTTA
TTTATGGTCTGACATTTGATAATTAAGCTTTCTCTCTTTTATTGCATTTTAAAATAGGATTAAAAATTTTTTTTGCCTTACGTAATTTTTAGTGTACC
ATAACTTTTACTGGAGTGTATATGGGAAAATGTACAAATTATAAATGTACAGCTTAGTGAGTTTTTACAAATTTAGCACCCACATACCCATGTAACCA
CTCAACCAAAATAAATAGATTACCAGAATCCCAGAAACAAGCATGAGTTCACGTTCAGTCCACTGGTTTCCATCCCTAAATGGGCCACAGTGGGGAAA
ATATGACACTTGTAACCATTATTCTGGCGTCTAACGTCATATTTGTAAAGTAACATACTAATCACATCTTACTGTAAAGTGAAAAAAACAAGGGACTC
TGAGAGAAAAATGCTAAGGACTCATTACTTAAAGTGAAATAATTAATTAAAGACCTTTTCCTAAGCAGTATTTGATACTAGGAATTGAATGTCTTTCT
ATTAAGGTAACATTTTTGGAAAAAGCTAATTTTTAGGCCAGGCGTGGTGGCTCACGCCTATAATCCCAGTACTTTGGGAAGCCGAGGTGGGTGGATCA
CCTGAGGTCCGGCCAACGTGGCGAAACCCAGTCCCTATTAAAAATACAAAAATTAACTAGGCATGGTGGTGCACACCTGTAGTCCCAGCTACTTAGGA
GGCTGAGGTAGGAGAATCGTTTGAATCTGGGAGGCAGAAGTTGCAGTGAACTGAGATTGCGCCACTGCACTCTAGTCTGGGTGACAGAGCAAGACTCT
GTCTCAAAAAAAAAAAAAAAAAAAAAAAAAAAGCTGATTTTTAGACATGTCTCTATTAGAGACAAAATTATTCATTTAATTCCGGGATTTGTAGTCAT
TTTGGAACTTTGCTGCTACTTAATTGCTAAATGACTAATGGGAATAAAGGGATGTCTTAGAATCATTACCATCCTTTGGTGCAATCATTACTAGACTA
TAATGCCCGTTTATAGTTTTTTTTGGTGGGGGACAGGGTCTCGCTCTGTTGCCCAGGCTGGAGTACAGTGGCGCGATCATGGCTCGCTGCAGCTTCAG
CTTCCTGGGCTCAGGTGATCCACCTCAGCCTCCCGAGTAGTTGGAACCACAGGCATAGCATACGCCACCGTGTCCAACTAATTTTTTTTTTTTTTTTT
TTTTTGTGACAGAGTCTCGCTCTGTCGTCCAGGCTGGAGTGCAGTGGCGCAATCTCGGCTCACTGCAAGCTCTGCCTCCCGGGTTCATGCCATTCTCC
TGCCTCAGCCTCCTGAGTAGCTGGGACTACAGGCACCTGCCACCATGCCCGGCTAATTTTTTTTTGTATTTTTAGTAGAGACGGGGTTTCACTGTGTT
AGCCAGGATGGTCTCGGTCTCCTGACCTCGTGATCCGCCCGTCTCGGCCTCCCAAAGTGCTGGGATTACAGGCGTGAGCCACCGAGCCTGGCCTAATT
TTGGTATTTTTTATAGAGGTGGAGTTTCGCCCTGTTGTTGAGGCTGGTCTTGAACTCCTGGGCTCAAGCGATCCTCTTGCCTTTGTCTCCCAAAGTGC
TGGGATTACGGTGTGAGCCACTGCGCCCTGCCTTTTATAGTCTTAATGTTAAAATTTAGCAGCATTTTACATTTCAAAACTGAGGCCTAAAACTTTCA
ACCTAACCTAAGTGGTATCTTTCTTAGTTACCCTTATATTCTATCTGGTTATCCAGATGAATGTAGAGAGAACTTTTAGTTCCCCATATTCAATTTTG
TTTTGTTGGCACTTTAGATTATCCTGAAGTCTTTTAGTTTCTTGAGTCTTCGTTTGTTGTTTGAAGAGGGTTTAACTTGGTAACCAGGATCTTTAAAT
TGGGCACAGAAAGATATACTGGATGGCTGGACAAGATAATGTATTTCTTTGATGATCATTACTAGATTTACTAATTGCTAACAGTTCATGAAGGTTTT
TTTGCAGTGTCTAGACAATGTCAGCCCAAGAGACTGAAATTCTAAATGAACAAACACTGAGAAATATTTGAAGAATTCATTCAGTAATTACCTATGTA
ACATTCTTGCTAAAGGTGTTTAATTTAAGCTGAGCTTAGTTATAGTAGACAATCAGACAAATCCAGTTTGTGGGACTTTCTACAAGACAGTTGGCCTG
AACTCTAAAAATTTCAAAGTGGTGAAAACGAAACGAAACAAAAAGGGCAGGAAGACTGTTCTAGATGAAAGAAGTTGAAAGAGAAATGACAGTCAAGT
ACAGTGTCAAGCCTGGATTAAGAAAACAAAAAACCATAGAGGACATTTTGGGGATAACTGGGAAAATTTGAATATGGTGTATATATTAGACGGTATTA
TACCTTTTTTTTGTTTTTTTTTTTTTTTGAGATGGAGTTTCGCTCTTGTTGCCCAGGCTGGAATGCAATAGCGTGATCTCGGCTCACCGCAACCTCTG
CCTGCCGGGTTCAAGCGATTCTCCTGCCTCAGCCTCCCGAGTAGCTGGGATTACAGGCATGCACCACCGCACTGGGCTAATTTTATATTTTTAGTAGA
GACGAGGTTTCTCCGTATTGGTCAGGCTGGTCTCGAGCTCCCGACCTCAGGTGATCTGCCCACCTCGGCCTCCCAAAGTGCTGGGATTACAGGTGTGA
GCCACCGTGCCTGGCCTATATCATTGTTAAATGTCTTGAATGTGACTATATGGTATTATGGTTATATAGGAAAATGATCTTATTTTTAGGCAATACCT
GATGAAGTATTAGTATGTTGAAATGTCGTGATTTTTGCAATTTGCTTTTAAATTGTTCAAAAAAAAATCATTTGTAGAGAAAAAAGCGAAAGTGGCAA
AACATTTACTATGGTGAATCTAGGTAAGAGCATATGGGTGTTCATTTTATGATTCTTTCACTTTTTAAATAGATTTCAGTTTTTCAAAATGTAAAGTT
GGGGACAGCCAGGCATGGTGGCTCTCGCCTATAGTCCCAGCTGCTTGGGAGGCTGAAGCGGGAGGATTGCTTGAGGCCAGGAGTTCCAAGGCTTCAGT
GAACTATGATTGCACTACTGTGTTCCAGCTTGGGTGACACAGTGAGCACCCATCTCTATTTAAAAAAGAAAAAAAAGTTAGGGAGAAAAAAAGTACAA
TGAAGAAATGGCTTGGCTGGGCGCAGTGGCTCATGCCTGTAATCCCAGTACTTTGGGAGGCCAAGGCGGCTGGATCACTTGAGCTCAGGAATTCGAGA
CTAGCCTGGCAACATGGTGAAACCCCGTGTCTACAAAAAATATAAAAAATTAGCCAGGTGTGGTGGCCTGCACCTGTGGACCCAGCTACTTGGGAGGC
TGAGGTGGGAGGATCACCTGAGCCAGGAGGTGGAGGTTGCAGGTTGCAGTGGGCCAAGATAGCGCCACACTCCAGCCTGGGTGACTGAGACCCCATCT
CAAAAAAAAAAAAAAAAAAAAAAAAAGGAAATGCCTCAAAACAGCTACAGCTAGGTTTAGGAAAGGCTGTCATCATATTTGAGGATTCCATAGTGTCT
TAATGGGGGAAAGCTGAAAAGAAGAAAATGAGCCTTAAAGAATATTATAGCTGATGTGAAGTGGAGGCAGCTTGTTTAGCAGAAAAAGTAGTGGTTTC
AGCTGCATGATGGGGGTTAGAAAAACATACAGAAGTCAGTATGGAGAAAGAAAAGGTACTTAACTAAGAAGTAGAGAGAAGATGGGCAAACTAGAAGG
GCCAGAGAACTGAAATACATAAAATGAAGAAAAAGAAGAGGGATAGTTGAAAACTGGTGGAGCAGTTGACATCAATTTCATTAATGGGACAGCAAGAC
TATTTTGATGACTGTTATTTTTTTCTTTTCTTTTTTTTTTTTTTTTCTGAGACAGAGTCTCGCTGTCGCCCGGGCTGGAATGCAGTGGCGCAATCTCG
GCTCACTGCAACCTCTGTCTCCCGGGTTCAAGTGATTATCCTGCCTCAGCCTCCTGAGTGGCTGGGATTACAGGTGTGCGCCACCACACCCAGCTAAT
TTTTTTTTGTATTTTTAGTAGAGAAGGGGTTTCACCATGTTGGTCAGGCTGGTCTTGAACTCCTGACCTTGTGATCTGCCCACCTTGGTCTCCCAAAG
TGCTGGGATTACAGGCGTGAGCCACCGCGCCCGGCTGTGATGACCATGTTTTAAAAACCCAAAACAAATAGACTTATAAACCTATTCTGAAATCATAG
AAAATCCAAATATTACCCAGACAGTTGATGTGGAAAGTGGAATAGCTATGTTTTTAATTTTATTGAAGAGATTGAAGTGAAAAAAGGGGTGGTAGTGG
GATAAAGTAAATATTAACACTAATGAGTAGAGGAGAAAAAAAGGATTTTCTAAGTGTATTTTTCATCCCTGTTAATTGCACCACTCACCCATTTAGTC
ACTGATTCAGATGACCAGCCTGGGAATCAGGCCAGACTCTTTCTTCTCCATTTTACTGCATGTCCAGTCCCACCAGACTAAATGCTGTGAACCTTTGG
TTTGTACATATCCTCTACTTCATCCTATTCTCACACCTTTTGCTTGTGCCATTGCAGGAGCCTCTTGCTGAGACTATGTAGTCTGTCCACGCTGTCAT
CGTGATCTGTGAACAATATCAAGGGTCATTCTTGTGCTCAAAATCCTTCATGAGCACTTCCTGCCTGTGGGATAAACTCTTTGTTGATGGGCAGTACC
CTGCATGCTAAACCTCTCTAGGCTTGTGTTCCATTTCTTCCTCCTCATTTCACAACCCTCATCCCCAACCTACTTTATTTTTATTTATTTATTTATTT
ATTTATTTATTTATTTATTTATTTATTTATTTATTGAGACGGAGTCTCGCTCTGTCACCCAGGCTAGAGTGCAGTGGCGCCACCACGGCTCACTGAAA
GCTCCGCCTCCGGATTCACGCCATTCTCCTGCCTCAGCCTCCTGAGTAGCTGGGACTACAGCCACGCGCCACCACGCCCGGCTAATTTTTCTTTTTTT
TTTTTTAATAGAGATGGGGTTTCACCGTGTTAGCCAAGATAGTCTTGATTTCCTGAGCTCATGATCTGCCCACCACGGCCTCCCAAAGTGCTGGGATT
ACAGGCGTGAGCCACCGCACCCGGCCCATTTATTTATTTTTGAGACAGAGTCTCACTCTGTCTCCCAGGCTGGAGTGCAGTGGCGCCATCTTGGCTCA
CTGCAACCTCCGCCTCCCAGGTTCAAGTGATTCTCCTGCCCCAGCCTCCCGAGTAGCTGGGATTACAGGTGCATGCCACCACGCCCAGCTAGTTTTTG
TATTTTTAGTAGTGATGGGGTTTCACCATGTTGGCCAGGCTGGTCTCAAACTTCTGACCTCAGGTGATCTGCCCTCCTCGGCCTCCCAAAGTGCTGGG
ATTACAGGCGTGAGCCACTGTGTCCGGCCCCCAGCCTCCTTTAATACTACAGGTTGAGTGTTCCTAATCCAAAATCTGACATTCTCCAAAATCTGAAG
CTTTTTGAGTGCCAGATGACACCACAAGTGGACACTTTCTTTCCTGACCTCATGTGATGGGTTGTAATCAAAACCTGGGGACATAACCCAAAGTTTTT
TCAGTGTCCCCAAGGGAAAAAGACCCTCCCAGCCCCCTTCGGTTGTGATATATCTTTTCCATGCACAGCATGATGGTGATGTCCAGGCAACCACAGAT
TGACCACGTAGGTGGCTAAGGGTAGTGACACATTTGCTTTCTAATTCAGTGTACGCAAATTTATTTTGTGCACAAAATTATTAAAAATATTTATAAAA
TTATCTTCACATTCTGTGTATATGAAACATAAATGAATTTTGTGTTTGGACTTGGGTTCCATTCCCAACACATCTCATTATGTATATGCAAATATTCC
CAAATCTGAAACAATCTGAAATTGAAATGCTTCTGGTCCTAAGCACTTTGAATAAGAAGTCATACTTGTCGTCCTAGAATGTTTTATATCTGTGAATG
GGCTCTTTGCTTCTGTGCATAATGGTTTTTTTTTTTTTTAACAGTTTTAGAGATAAGGCCTCACTCTGTTGTCTAGGCTGGAGTGCAGTAATGCGATC
AATGCTCACTGTGGCCTCAAACAAACTCCTGGCCTCAATTGATCGTCCTGCCTCATCCCTCTGAGTAGCTGGGACTACAGGCGTGTGCTGCCATGTCT
GGCTAAATGTTTTATTTTTATTACTTTTTTGTAGTGACAGGGTCTTGCTATGTTGCCCAGCCTGGTCTCAAACTCCTGGCCTCAAGTGTTCCTCCCAC
CTCCCAGAGAGCTGGGATTACAGGCATGAGCCACTGTGCCTGGCCCTGCGTAGAATCTTGTCCCTCAGCTTGACCAGCTCCTGTTTGTTTTTCAAGAT
TTAGCCAAGGGGACTTCTGAGAAGCTTTTTCTTCTCTGCTAAGAAGCTGTGGAGAGTAATGGTTTGAGAACATGGGCTTCAGTCAGACATACCTGGAT
TCAAATTTCTGTTTGTTTTTTTTTTCAAGGAGATGGAGTCTTGCTCTGTTAGCCGGCTGAAGAAGTGCAGTGGCACCATCATAGCTCACTGCAGCCTC
AAACTCCTGGGCTCAAGTGATTCTCTTGCCTCATTCTCATGCCTGGCACTACAAGCATGTGCTACTATGCCCAGCTCAAATTCTTTTAGTGATGCATG
ACCTTAGATAAATCACTTACACTTGTTTTACCTGCATTTCTTTATGTGTAGAAGAGGGATAATGAAACTTGCAGTGGCAAGCACATAGTGAGTACTCC
ATATATGTTATGGTAGGTGCTGCTGCCGCCATCGCTATCATCATCATCATCATAAGAATTACTGTCTCCTATGAGCTCCCATAAACAATCTCATTGCT
TTGCAAATCGCTTTAAATATTTTCTTGTCCTTTCTTCACAATAAACTCCTCTCTTTAAGGGCAACCACTGTATCCTTAATGCTGAATGCAGTGCTTAA
CACACGCTTAGGTGCTTAGTAAATATTTAAGTATGTGCAAGCATAAGAAGCTTATTTTATGGATGACAGTGTAAGAAAGAGTACAGACTCTGCTGATT
ACCAGCCGTGTGGCCTTTGAGCTTTGTTATTTATTTTCCTTGCCACAGGTGTAAAATGGGAGATAGTGCCTACCTCAGAATTTAAAGATTACATCTCA
TATTGTATGTAAAGTGCTTAGCATAATGCCCATAGTGGGTGGTTAATAAATGTTAGCTATTATTTAAAAAATAAATCAATCTTGGATCTTGGCGAGAT
TTGACAGTAGATCTATTTTACTTTAAGTGACATGTGAAATGAAGATTTAAGAGTACTGTATTTTATATAGTCACGCTTACATAGTTGCTTAATAATTC
GGAGTTCTGACAAATGCATCCTTAGGCGATTTCATCATTATGCAAACATTGTATAGTGTACAGTTATCCCTTGGTATATGTGAGAGCTTAGTTCCAGG
ACCCCCACATATACCAAAATCCCTGAATATTCAAGTCCAGAAGTTGGCCCTGTGGAACCTGAGAATTCAAAAAGTTGGCCCTCTGTATTTGCAGGATG
GGGTTTTGCAGCCTGTGTTTGGTTGAAAACATTTGCATATAAGTGAACCAGTGCAGTTCAAACCCATGTTGTTTGAGTTAACCATACTTATACAAACC
TAGATGTTATATATATCTACAAACGTAGATGTTACACACCTAGGCTGTATGGTATATAGCCTATTGCTCCTAATCTACAAACCTGTGCAGCATGTTAC
TCTATTTAATACTGTAGGCAACTGTAACACAACAGTAAGTCTTTGGGTATCTAAACATATCTAAACCTAGAAAAGGTACAGTAAAAGTACAGTATTAA
AGATAAAAAATGGTCCACCTGTGGAGGGCACTTAACATGAATAGGGCTTGCAGGACTGGAAATTGCTCTGGGTGAGTCAGGGAGTATGTGGTGAGTGA
ATGTGAAGGCCTAGGGCATTACTGTAGAACACCGCAGGCTTTACAAACACTGCACACTTAGGCTATGCTAAATTTATAAAATATTTTTCTTCAATAGT
AAATTAGCCTTAGCTTACTGTAACTTTTACTTTATAAACTTAAAAATTTTTAAACTTTTTAACTCTTGTACTCATACTTAGCTTAAAACACAAACACA
TATAGCTGTACATATTTTCTTTCATATCCTTATTCTTATAAGCTTTTACGATTTCAAACATTTCTTATTTTTAAAACTTTTTTGTTAAAAATGAACAC
ACACCACATACATTAGCTGAGGCCTCCAGAGAGTCAGGATGATCAATGTCACTGTCTTCTACCTCCACCTCCTGTCCCACTGGAAGGTCTTCAGGGGA
ATTAACAGGCATGGAGCAGTCACCTCCTATGACAACAATGCCTTCTCATGGAATACCTCCTGAAGGACCTGTCTGGGGCTGTTTTACAGTTACCTTTT
TTTTGTTGTTTTTGTTTTTTGAATAAGTAGAAGGAGTATACTCTAAAGTAATGATTAAAAGGGTGGTATAGTGAATGTATAAGCCAGTAACATATTTG
TTATCGTTATATGACTGGCAGTGCAGTAGGTTTGTTTACACCGGCAGTACCACAAATGTGAGTAGTAGTGCCTTATGCCGTGATGTTAAGACAGGTGA
AAACATCACTAGGTGATAGAAACTTTTCAGCTCCATTATAATCTTACAGGATCTCTGTCATATATATATAGTCTTGTAGACTGAAATATTGCTATGTG
GCACATGACTGTTTTCTAACTTTAGTCTTAGATATTGAAAGGATTTAAGGCTGGGCGCGGTGGCTCACGCCTGTAATCCCAGCACTTTGGGAGGCCGA
GGCGGGTGGATCATGAGTTCAGGAGATCGAGACCACGGTGAAACCCCATCTCTACTAAAAATACAAAAAATTAGCCGGGCGCGGTGGTGGACACCTGT
AGTCCCAGCTACTCGGGAGGCTAAGGCAGGAGAATGGCATGAACCCGGGAGGCAGAGCTTGCAGTGAGCCGAGATTGCGCCACTGCACTCCAGCCTGG
GCGACAGAGTGAGACTCCATCTCAAAAAAAAAAAAAAAAAAAGAAAGAAAGGACTTAAACCTAAAATGCTTAAGAAGAAAAATGATGAAATTTTCCTT
TTCTATAGTATAGATAGAATTTTGGAAGTGATGTATTCATAAATATTTGATGAGTTCCTATTACATGGAGCACATTTCTGGTTATGCTTAGAAGGGTT
AAATACTACTTGAAAAGTACTCATGCGGTGTAGCATATAATAGCCTCTCAAAAATGTCACAGAAATTTAGAACTATATAGTGACCTATGACTAATCCC
ACTGCAAAAGGAGCAGGTGACTAAATTGTAGACAATACCTGGTGTGAAATGTTTGTTCCCTTGATCTATAAATTGATAATCCTTTAATGTTTAATCTG
ACTGTGACTAGTGGCCTGAAGAGGTAAGACTTTTGTGTTTTTTTATGTGTGTGCCTACGTATGTTTTTTTCTTGTGACCTAAGGCTTTGTGGAAGTTT
AAAATAGAGTGATTTTAATGAATGGCTGGAGTAATAAAGATAGTTGGTGGGCTTCTCCATGGAAGCAAGTTGGTTATTTTTAACTACTTTGCACTGCT
TTATGGAAAACATATGCCACTATAGAAGTTATTGTTTCTGTTGACTGTGACTGGTGGTGGTTATTACTTAGTTGGTTCTAGGAGGGCCAGTTTTTAGC
TAATACTTTGAAAGTGAGTTCTGTGACTATTATTACTTAGTTGGTTCTAGGAGGGCCAGTTTTTAGCTAATACTTTGAAAGTGAGTTCTGTGACTATT
ATTACTTAGTTGGTTCTAGGAGGGCCAGTTTTTAGCTAATACTTTGAAAGTGAGTTCTGTGACTATCAAAGACCAGAGGAATAATCTCAAAGAAGCTG
ATCAGATCATGGAACTAAAGTCACTTGTTCTGGTGAGGAACAGTTGAAAGGGCACATAATACTTTTGGTAATAATATTTAATGTAACTTATTTAGCAG
TGCGTGTTCATGATTGGCAAGGCAGGTGAGTAGATTAGGAAATGAGTCTGAAAAAGTAGGGCAGGACTGCTGGCTTAAAATAGGAGTGAAAGCAGTGA
ACAGACATAGCTATTTAGGCAAATCCTAGTGTCCCATGAGGATAAGACTACCACAATAATCTAAAAATAATAGCTTATAGGCCAATAACCTTTAAGGT
TTATGTTTCATACTCACCGCAGGTCAACCCATGTGGCTGTTTCTCATTTATGCAGATCTACATCAGATTTTACTTGTGTTTACCTGAAAATACATATT
GTTTATGTAGTCAATGTATATTGTAGTAGCATACAGAATCTGATCTTGAAAAGGTTGCAGAATTTCTTGAGTGTACTTTAGGAACTTAGTTTTTCAAA
ATTTATTTAATTTTTATTTTGATAATTATAGATTCACAGGAGGTTGTAAATAAAGAAATGTAAAGGGAGGTCCCAAACACCTCTCCCCTAACTCCCTT
CCCTCTCTTTGCCCGTGTCAGTATCCCACACCACCACACACAGTAGTATACCAAAACCAGGAAATTGACATTGGTACAGTCCATAGAGCTTGTTCAGA
TTTCACCAGTTATACATGCATTCCTGTCTGTGTATGTGTGTTTAGCTTTACACAATTTCACATATAGGTAGCATAACTACCAGCACAATAAGGGTACT
CAACAGTACCGTAACCACAAGATTTAGTTACTTCCCCTGTATAGCTACACCAGTCCCCCTTCTCCACGGGAAACTCAGTTTTATAGTTTGGCTATTGT
TTTTAATCATGTGCAAACCTGGTTTGATTAAGGTTTTTGCTTCTGATTTGGAAGCATTGTTAAATAATTTTTAATGAAATACAGGTAATCTGAAAGCA
GCTGACACATGGTAAAAAGCTAGATTTGGGAACACTGAAAGACTTCTTGGATGCATAGGCTTAGAATGATTCTCAGCTGTGATTTATTTCCTCCCTCC
CTCCCTCCCTCCCTCCCTCCCTCCCTTCCTCCCTTCCTCCCTTCCTCCCTCCCTTCCTTCCCCTCCCTCCCTCCCTCCCTCCTTCCCTCCCTCCTTCC
TTCCCTCCTTCCCCCTCCATCCCTTCCCCTCTCTCAATATTCTGTAATATTGAAATATTTCGTTAAAAATATACAGCATATTACATATATATCTGAAA
TTTTCTCCTTTCTTCCTTTTAGTATACCTTCTATTTATTTTGGTACCAAGAATAGAGTGCTGCTGTAACAAACACCTAAAAGTGTGGAAGTGGCTTTG
GAACTTCTGAAAGACCCAGCCCAAGTCCCATCTTCATTTATTTATTTATTTATTTTTAAGAATTTTTTTTCCAAGGCTGGATCTCGGCTCACTGCAAC
CTCCGCCTCCTAGGTTCAAGTGATTCTCCTGCCTCAGCCTCTGGAGTAGCTGGGACTACAGGTGCACACCACCATGCCCGACTAATTTTTGTGGTTTT
TAGTAGAGATAGGGAGGGTTTCACGATGTTGGCCAGGCTGCTCTTGAACTCCAGGCCTCAAGCGATCCATCTGCTTCATCCTCCCAAAGTTCTGGAAT
TACAGGCATGAGCCACTGCACCCAGCCCAAGTCTCAACTTTCTCATACACTTAATTGATTTCTGTAGCTATCGTTGATTTCCCCCTTTATAATCTTCC
GTACTTACAGTCTGAACCATACAATGTACTGCTTAACTATTCTGTGTTTATATCTTGTTTTATGCGAATTCTACTCAACTAGAGCATATGATTCTTCA
AGGTAGAATAAATGTGTTAAACTTGTATATTTCGGCCGGGCGCAGTGGCTTACGCCTGTAATCCCAGCACTTTGGGAGGCCAAGGCGGGCGGATCACC
TGAGGTCAGGAGTTCGAGATCAGCCTGGGCAACACGGTGAAACCCCGTCTCTACTAAAAATACAAAATTAGCCAGGCGTGGTGGCACATGCCTGTAAT
CCCAGCTACTCAGGAGGCTGAGGCAGGAGAACCGCTTGAACCTGGGAGGCAGAGGATGCAGTGAGCCGAGATCGTGCCATTGCACTCCAGCCTGGGCA
ACAAGAGTAAATCTCCGTCGCACCAAAAAACAAAAAAAACAAAAAAAACACACAAAAACAACTTGTATATTTCACACATTACCCATACAATGTGAGCA
TGTAGTAGGTACCAGATAAACATTTAGTTATTTAACTAGAATATAATTACTGCTCTAGTATTTTTTCCCTAGCACTTTCAAACGTTACTCAAAGAATC
CTTCTAGCTCTTTGATACAAATTTTAGAACTTCACTATTAACAAATATCAGCTGGGCTCGGTGGCTCACGCCTGTAATCCTAGCACTTTGGGAGGCCG
AGGCAGGCGGATTGCTTGAGGTCAGGAATTTGAGATCAGCCTGACCAACATGGTGAAACCTTGTCTCTACTAAGAATACAAAAATTAGCTGGCCGTGG
TGGTTCTCAACTGTAATCCTAGCTACTTGGGAGGCTGAGACAGGAGAATTGCTTGAACCTGAGAGGCAGAGGTTGCAGTGAGCTGAGATCATGCCACT
GCATTCCAGCCTGGGCAACAGAGCGAGACACCGTCTCAAAAAAAATAAAAATAAAAATAAAAATAAATAAATAAAATAAAGTAAAATAAAAATATATA
TATATCACTACTAGCTAGGATTTTAGGAAGGCTACCTATAGAAACTTAGATATATTTAGTTATGGAATGTTTTGTTTTTCAGTTGGTAATGTACACTC
TTTCAGTGTTTCAAAGGAGGATACTGGATAATTCGATTTGAATTGTAAATCTCCCCAACTGGTGTTGGAGAAACCAGAGGAGTTGATGATTTTATACA
CAGTCTTTCCTACCTCTTATTATTGAGAAAAGAGTGTTAATTGTGGTCAATGAATTTCTTCAAAGTTTGCTGCTTTTAAATCAAATTAAGGAAGTCAG
CAACTATAAGTAGAATGATATGAATAAAATGTTTCATTGTTATGAAGAAGTGTTAGTAGCACGTGGCATTATGTGTTTCACATATTGAAGTTGACACC
AGAAATATACATTTGTAATTTTTTTTTTTTTTCTGAGACAGAGTCTAGCACTGCTGCCCAGGCTGGAGTATAGTGGTGCAGTCATAGCTCACTGCAGC
CTGGAACTCCTGGGCCCAAGTGATCGTTCCGCCTCCACCTACACCTCCCAAGTAGCTGGAATTACAGGCACATGCCACTGTGCCCAGACCATTTTAGT
GATTTGTTATTTTTTAAATCTCTATTCTCTTTTTAATACAGAGCTGCAAATTTCTTACTTTTCTTTAGTGCAATCCATGTGTGAAATATGTGTTATAA
TTTGTAACTAACAATATTTGAAGTCCTGAAGTTTTTGAATGATCTTGTATGTAAGATTAGAGTAGATTCATGGTTCCCACTAATAACCACTTCATTAT
TATCACTTTGTTATGTGTGTGTGTGTGTGTGTGTTTAAAGAGATAAACTTATAGGTGGTTTATCTAGAATTGTGTAAAATACTTTCATTTTCATGAGA
GAGTATCTTGCATGTGAGTATTATTTGTCACATGTTATGCCTACATTGTTGGTTGGACTTAAACTTTAGTGACCTCTCAGAGATTATTTTCATTCTAG
TTCATGTCATATTACTTTGATATATGTATTAAGAAGAAGTACAAGAGATTATTTAATGCTATTGTCTGAATGTTTGTGACCCCCCAGAATTTGTATGT
TGAAATCCTAATCCCCAATGTAATGGTATTAGGAGGTATGGTCTTGGGGAGGTGAAGTTTTTATAAATTTCTAAAAGAAAGTATAGGAAAAATCTTTG
TGACTTTGGGGTAGACTATTTTCTTAGATAGGTCATAGAAGGCATGAACCATAAACGCTGACAATTGGACTTTTGAAAAATTGTTCTTCTAAAGATGT
TTAAGAAAATGAAGAGGCAAGCCACAGAATGGGAGAAAATATCTATACTGCATTAGTGGAGTTTACTACTAAATTAAGAAAACATTAGAAGATTGTCT
ATATAGTATAACTGATTATAAAATAATGTATTTACATATTTAAAATATTAAGATATATACTGACATCTTAACAGTAGTTGTCTTTGGCTGGTGAGATT
CTAAGAGTTTTGTTTTTGTGGTGGTTATTGTTTTTTGTTTTGTGCTCTTTTAAAAAATTGTTTTGCCTCAAGTGTATAGTATCTTTGATTTATAAAAA
TCTTTAAATAAAGATAGGATAGTTTGAAAGATTAGCAGAATGTTTACAATCAATACTTGATACTACATGGATAATACTTTCCTATTCTATTTTTGTAA
ATTCTGTGCTTGAAAATTTCAAAGAATTTTTTGTGTGTGAGTGTGTGTTTTGAGGTGGAGTCTCGCTATGTTGCCCAGGCTGGTCTCAAACTCCTGTC
CTCAAGTGATTTTCCCATCTTGGCCCCCTGTAGCATTGAGATTACAAGCATGAGCCACCATTCCCAGGCCAAAACTTTCGAAACTTTAAAAAATAGCA
TGAAAACCTTTATTTTAGTCCAGGTGCAATGGCTCACATCTGTAAACCTGGCACTTTGGGAGGCTGAGGTGGGAGAACTGCTTGAAGCCAGGAGTTTG
AGACCAGCCTGGGCAACATGATGACACCTGTCTCTACAGAAAATTTAAAAATTAGTCAGGTGTCATGGTGCATATCTGAAGTCCCAGCTACTCTGGAA
GCTGAGGTGAGAGGATTGCTTGAGGAGTTCGAGGCTACCGTGAGGCATGATCAAGCAGCTGCACACCATCCAGCCTGAGTGACAGAGTGAGAGACCCT
GTGTCTAAAAATGAAAAAAGAAAATCTTTACTTTATTTATTTATTTTTTGAGATAGAGTCTTGCTCAGTTGCCCAGGCTGGAGTGCAGTGGTGTGATC
TCGGCTCACTGCAACCTCCACCTCCCAGGTTCAAGTGATTCTCCTGCCTTAGCCTCCCGAGTAGCTGGAACTACAGGTGTCTACCACCATGCCCGGCT
AATTTTTGTGTTTTTAATAGAAACAGGATTTCACCATATTGGCCAGGCTGGTCACGAACTCCTGACCTTGTTATCCGCCCACCTTGGCCTCCCAAAGT
GCTAGGATTACAGGCGTGAGCCACCGCACCCAGCTGAAAATCTTTACTTTATTTACAGCACTTTGTGAGCCATATTTAGCAAACATAGTTCCACATAT
GAAGTTTGTTTTTAATGATAAGGGGTTTTTAAACTTGATTTTTAGTATAAATACACAGTATAATAAAAGTTTGTTTACATTAGATATGAAGCTAATTA
CCACCCCTAACTGCTGGCTGGTTTTCTAGAATTGTAGAAGCAGGTGGTGAATTTTCCTGTCTGCTGATATCCAAAACACTCTGATTCCTGGGTAAGTT
CCAGCAGTATTGGATGTTGGTAATATTACTGTTGGTCGATTAATTTATGATAGTATTATATACATCTCTAAAGTCTAATTCTATGTATTTTTTAAGTA
AATAGTAGAATAACATGGCATAACATTTTAAAAGGTATGTGGTAAAAAGTCTTCCTATTCCTAACTGTCAGCCACCAGTTTCCTGCCATGGGACTGTT
AAATCTCACCACCAAAGACCACCAAGAAAACATCTGTAGTCAAAAATAAGTTACACTGATTTAGCTTGCTGCAGGAAGGAATAACATACATCAGAAGA
GCTGTGGGACCTCTCAAAAGGATAGAGTTAGGAAAGAGGACATACAGGATTTTAGGAGCTGTTAGTCACAGAGTAATGTTAGGATAAAGGTAGTAGTC
TGGGCAGGGATTGGCTAGAATATGTAATTTGAGTGAGTTGCTGGAACAGCCAGTAGTTTTGCCTCTAGGACACATAAGTCCCTGAGGATTTACTGTTA
GATCAGTTTGTGGTACTAGAACATACGGGTATGAGTGAGCTGATATTAATAAAGGTTTGAGGCTGGGCACAGTGGCTCACACCTTTAATCCCAGTGCA
TTGGGAGTCTAGGATGGGAGGATCGCATGAGGCCAGGAGTTTGAGACTAGCCTGTTTAACTTAGTGAGACCTCAGTCTTTAAAAAAATTTTTTTAAAA
TTAAAATTAACTGAGTGTGGTGGCACACACCTGTAGTCTTAGGTACTTGGGAGGCTGAGGGGGAGGATCACTTGAACTCAAGAGTTGGAGACTGCAGT
GAGCTATGATCATGCCACTGCACCCCAGCCTGGGTGACAGAGGGAGACCCTCTCTCTTAAAAAGAAAAAAAATAAGGTTTGAGCTTAGATGGCCTGTG
ATGCACAGTACATTGCGGTTTGGTTTGGTTCAGTTTATCTGTTTTGCAAGTACTAAGTGCTTTTCTGTGCATCTGCAAGCAATTTGTAGTGTAATTTC
AGTTTTGTTTCACTTTTTAATCTCTGTTTCTGATCATTCGTCCGCTAAACTGGAACATTTACCATTTAGGGGAGGTGATCAATCAAGATTTCAAACTG
TCGTTTGATGGGTTGTGAATGTTTATAAAGCTTTGATGGTAGACATTTTGGCCTCTACAGGCACCAGGCCATTTTGTTCTTTTTTTTCTTTTTTCTTT
TTTTTTTTTTGAGACGGAGTCTTGCTCTGTTGCCCAGGCTGGAGTGCGGTGGCACGATCTCGGCTCACTGCAAGCTCCGCCTCCTGAGTTCACACCAT
TCTCCTGCCTCAGCCTCCTGAGTAGCTGGGACTACAGGCACCCGCCACCATGCCAGGCTAATTTTTGTATTTTTAGTAGGGACGGGGTTTCACCATGT
TAGCCAGGATGGCCTCGATGTTCTTTTTCTTAGATGATAATTTGTTGAATCAGCTGGTGTGACAGTGGCTGCACAGCAGCATTTAAGACTCTAAAGAT
CACACACTTGGCATCTGCAACACACTGAAACTCAGACAGTTATTATTAAAGTTTAAAATAATGTCCCATATATCCCATAATCTATTTGGATTTTCCTT
TGGGCAACCAAATATATTTTTTTTCCTTTAATTTGGATATGGGTCATTCACCTTTGCAACTGTCCCATATTTGGTTTATATTTTGCCAAGGATAAGCA
TCTTGATAGTGCTAACAGTAAGGAAAAGTTATCTGTTAGCAGCCTAAAATCTTACAACAGTGGAGAGAGAAGAAAATGCCAAGAGAATAAAGGACAAA
ATCATGCAAGTCTGTAGTCATCAGTGGCTTCTGGCATCTGGGAGGTTGTGTTCTGATTATCTTGGACATAAATAGTCTATTCATTTTATCTTCTGCTT
CTGGACCATAACAGTTCTGGAAATCTTGGCTCACCCACTGTTTGTCCATGGCAATGATGTCGGCAGTATGCATCATTAATTTATTCTCTGTAGTGTCA
GTAATGGGTGCATGATACAAACATTTACAAAAGTTCTTTTGATAAGTCTTCCACAAGGCTCAATTCCTATCTTTAGCTTATAGCCAGGAACTTTAGAT
GAATGTAAGGAAGAAGCGGAAACCACCTGTGGATCATGGGACTATAGGCTTTAGTTGATTTATTATATTAACCAGCATTATCAGAATGGAAGGGAGTG
GAATCTTCTATTGCAGAAAGTACATAATGAATTTTATGAGTAGTCACTGATTATCCTAGAAGTAAGAATATCTCACAAACAGGGAGGACATTGACAGT
CTCTAGGACTTTCTTTCATTTATCCTATAAAGTGTATTATGAGTTTCACCATTCCAAGTTTACTGAAACCTAGAAATAAAATCCTTTTTTAGATAGCT
ATTGTGGCAGTCTTTGTACTCGAATGTATGTTTTCTTTTTTTTTTTTTTGAGACGAAGTTTCTCTCTTGTTTCCCAGGCTGGAGTGCAATGGCATGAT
CTCGGCTCACTGCAACCTCCGCCTCCTGGATTCAAGCAATTCTCCTGCCTCAGCCTTCCAAGTAGCTGGGATTACAGGTGTCCACCACCAAACCCCGC
TAATTTTTTGTATTTTTAAGAGAGATGGGGTTTCACCATGTTGGTCAGGCTGGTCTCGAACTCCTGACCTCAGGTGATCTGCCTACCTCGGCCTCCTA
ATATTTTCTTTAAGCCGAAAGATACTTAAATGTTGAAGTATATTTATTTTAACCACATTTTATTATACATGATTAAATTGGGAAGGCTTAACTTTCCT
TATGATTTGTGAGGTTTTTTTTTCCCTTTAACCCAAATTTAATAAAGCAAAGAGTTCTCACAAAAGTTTTAAGTTAATTAAAATTCAGAGTATGCCAA
ACAAAGCTAATAATTTTTATAATCCTTTAAAAAATTTTTAATAAAGTAAAGGACTAAAGCTTTGTTTTACCTAGTGGCCATTTATTATAAACCCAAAG
ATGTAGTAGGTGTAAAAGACATCTTAACAATTTCTTATTATTTACTTATTTTTTAGAGACAGGGTCTCGCTCAGCTGTCTAGGCTGGAGTGCAGTGGC
TGGATCATAGCTCACTGCAGCCTCAATCTCCTGGGCTCAAGTGATTTTCCTGCCTCAGCCTCCTGAGCAGCTGGGACTATAGGTGTGCACCACCACAC
TCAACTAATTTTTGTGTTTTTTTGGTAGAGACAGAGTTTCGCCATGTTGCGCAGGCTGGTCTCGAACTCCTGAGCTCAAGCAATCCACCCACCTCAGC
CTCCCAGAGTGATGAGATTCTTTTTATAGGTGTGAGTCATCATACCTGGACTACTTTTTGCTTTTTGACTGATGATTCCAAGAGCCTTTTCAGTGTTT
AGCACATAGACGTAGATAGTTGTAGTCTTTACTTAAGTTTGAATGAAGTGAGTTATGCGTGGGCTGAGTTCACTCAAAGCTTGCCTCAGCTGGGATTA
TTGACCATATACCCACTTTACTGGAGAATGAGTATGCCAAGAGACCGAGGCAGAATCTGCAAAATCACTTCTGCTGCAATGTATTTGTTAAAGCAGGT
CACTAAGGCCAGTTCAGATTCTACGGGAGAGACATTAGACTCTCCCTTTTTTTTTTTTTTTTTGTCACCTAGGCTGGAATGCAGTGGTGCTATCTTGG
CTCACTGCAGCCTCGACTTCCTGGGCTCAAGTGATCCTCCCACCTCAGCCGCCCAAGTAGCTGGGACTCCAGGCTTGTGACACCACATTCGGCTAATT
TTTGTTGTGTTTTTAGTAGAGACGGGGTTTCACCATGTTGCCCAGGCTGGACTCAAACTCCTGAGCTCAAGCATTCTGCCTGGCTCAGCCTGCCAAAG
TGCTAGGATTACAGGCATGAGCCACTGGGCCTGGCCTTAGACTCTACTTCTTGATAGGATAGGGTCAGGTCACTGCTATGCTTTAAATATGATCCCCA
GAAAGCATATGTTGGCTGCCTGGGTGATGGGTATACTAGAAGCCCAAACTTTACCATTATGCAGTATATCCATGTAAAAAAGGTACACATATACCCCT
TGAATCTGAAATTTAAAAATAAAATAAGTATGTATTAGAAAATTTATCCCAAATGCAGTAGTGTCGGGAGGTGGGGCCTAATGAGAGGTGTTTAGGTC
ATGAAGCCACCTCTAATGAATGCATTAATGTTGATTATAAAAGGGCTTAAGGCTGCAAGTTCTATCTCTTGCTCTCTCTTATCCCTCTTTGCCCTTCC
ACTATGGGATGATGCAGCAAGAAGACCCATGCCAGATGCCTGGCCCCTCAATCTTGAACTTCTTAGCCTCCAGAACTATGAATGAGTAAATTTCTATT
CATTATAAATTATCCAGTCTATAATATTTTGTCATAGCAACACAAAACAGACCAAGACAGTGAGATTACAGAAGAGTATGTAGAGTGAGAAATATTAT
TGTAGCCATTTTTTTTTAGTTTTTGCTTTTTTTAAAAAATTATTCTTTAAGTTCTAGGGTACATGTGCTCAACATGCAGGTTTGCTACATAGGTATAC
ATGTGCCATGTTGGTTTGCTGCACCCACCAACTCATCATTTACATTAGGTATTTCTCCTAATGCTCTCCCTCCCCCAGCCCCCCATCCTCCGACCAGC
CCCAGTGTGTGATGTTCCCCACCCTGTGTCCATGTGATCTCATTGTTCAATTCCCACCCAAGAGTGAGAACATGTGGTGTTTGGTTTTCTGTCCTTGT
GATAGTTTGCTGAGAATGATGGTTTCCAGCTTCATCCATGTCCCTGCAAAGGACATGAACTCATCCTTTTTATGGCTGCATAGTATTCCATGGTCTAT
ATGTGCCACGTTTTCTTTATCCAGTCTATCATTGATGGACATTTGGGTTGGTTCCAAGTCTTTGCTATTGTGAGCAGTGCTGCAATAAACATACGTGC
ACATGTGTCTTTATAGTAGAATGATTTATAATCCTTTGAGTATATGCCTAGTAATGGGATTGCTGGGTCAAATGGTATTTCTAGTTCTAGATCCTTGA
GGAATCACCACACTGTCTTCCACAGTGGTTGAACTGATTTACACACCCACCAACGGTGTATTTCTCCACATCCTCTCCAGCATCTGTTGTTTCCTGAC
TTTTTAATGATCACCATTCTAACTGGTGTGAGATGGTATCTTATTGTGGTTTTGATTTGCATTTCTCTGATGACCAGTGATGATGAGCATTTTTGCAT
ATGTCTGTTGGCTGCATAAATGTCTTCTTTTGAGAAGTGTCTGTTCATATCCTTTGCCCACTTTCTGATGGGGTTGTTTTTTTCTTGTAAATTTGTTT
AAGTTCTTTGTAGATTCTGGATATTAGCCCTTTGTCAGATGGGTAGATTGCAAAAATTTTTTCTGTTTTTGCTTTTTGAGATGGAGTCTCACTCTGTT
GTCCAGGCTGACGACTGTAGCTGACTGCAGCCTCAACCTATTGGGTTCCAGAGATCCTTCTGTGTCAGCCTCCTGAGTAGCTGGGAGTACAGGCAGAT
GCCACCACACCTGGATAATTTAAAAAAAAAATTTTGTAGAGATGGGGGTCTCACCATGCTGCCCAGGCTGGTTTCGAACTCTTGGCTTCAAGCAGTCT
CCCCAACTTGGATTCCCAAAGTGCTGGGATTACAGGAGTGAGTCACCATGCCTGGCTGGTTGCAGCCAGTTTTGGAAAATAAAATGTGCTACAGACAT
TGTAAGGGCAAAATATTTAACAATAACTGAGATTTCTAAAAATTGCAGATTGGCTAAATGTTAATAAATTATGATCTAAGTAAATTATATCTACCTAG
GAGAAAATAAAAAAAAATTTATGGTATTTTAAGGTATTTATTTATGTACTTATGCATTGAGATATTTGCCATCAAAATTTTTACTAGTGCGTATAAGT
ACCTAACACATAGTAGCAATTCAATAAAATTATTTGAATGAATTAATTGAGGATACTTTTCGATCATAGTTTTTATGGTGAGAGTTTTCATTTTGTTC
TGTCTTATTTTTATGTGTATTTTAGTTAGATGTTCGCTTAGTCTATTCAAGATGTGTTTTTTGTTTGTTTGTTTGTTTTGAGACAGAGCATTGCTCTG
TCGCCCAGGTTGGAGTTTCAGTGGTGCGATCTCAGCTCACTGCAACCTCCACTTCCTGGGTTCAAGTGATTCTCCTGCTTCAGCCTCCTGAGGCAAGC
ACGCGCCACCACACCCGGCTAATTTTTTATTTTTAGTAGAGATAGGGTTTCACCATGTTGGTCAGGCTGGTTTTGAACTCGACCTTATGATCCACCTG
CCTCCGCCTCCCAATGTTCTGGGATTACAGGTGTGAGCCACCGCCCCCGGCCTGTTCAAGATGTTATAACAAAATGCCTTAGACTGGGTAATTTGTGA
ACAACAGAAATTTATTTCTTACAGTCCTAGAGGCTGGGAAGTCCAAGAGTCAGCTTGCCAGCAGATTGTGTGTCTGGTGAGGGCCTCTTCCTTATAAG
TGGCACCTTCTATGTGTCCTCACGTGGCAGAAGGGACAAACAAGCTCCTTTGGACCTCTTTTGTAATGGCACAAATGCCATTCATGAGGGCAGAGTCC
TTACGACCTAATAATTTCCCAGATATTCCACGTCTTAATACCACCATGTTGGGGATTAGGTTTTACCTATGAATTTTTGGAACATTCAAATCATAGCA
GCTGTTAAGAAATGTTGAATCAGAAGTTTCTAGTCATTCTTTTTTTTGTTTTTTTGAGGCAGAGTCTTGCTCTGTCGCCCAGGCTGGAGTGCAGTGGT
GCAATCTCGGCTCACTGCAACCTCTGCCTCCCGGGTTCAAGTGACTACCCTGTCTCAGCCTCCCGAGTAGCTGGGATTACAGGCGAAAGCTACCACGC
CCAGCTAACTTTTGTATTTTTGGTAGAGGCAAGAGTGTCACCATGTTGGCCAGGCTGGTCTCAAACTCCTGACCTCAGGTGATCTGCCTACCTTGGCC
TCCCGAAGTGCTGTGATTACAGGCGTGAGCCACTGCACCTGGCCAGAAGTTGCTAGTCATTCTAAACTGGAAGCTTGACATAATATTTTGAATCAAGC
ATTTTAAAAATGTCTGCTTTTCATAAATAGTTTATTTTTACATATTTATCCCTTTCATTTAGGAAAGAAGATTCATGAGTTTTTTTTTTAATCAGCTG
GCAAATTAGAGAAAATATTTATACAGCTTCTAATAGGCATCTCAAATTATTTTGAAACATTGGGAATATATCTTTTGTAATGTTTTGCTTGTAATAAT
TGAATAGATTTTCTCATTTGGTAATAACTTGGAAAATACAAAGTGGCCCATTTAAAATGGACTCTGATTTAAAGGTACTGTTGGAGGCATTGGTGACG
GGCCATTTTTCCTTTTAAATAGAGAAAGTATGACAATCTATTTTCAAGAAACTGCCATAGCAATATTTTTAGTCTCATATGCTCGCACAGAATTTTGT
CACTCCCCACCAGTAGGTGGAATCTATTTCCACTCTTCTTGAACCTGGGCAGGCTTGTGACTGTTCAACCAATTGAATATGGTGGAAATAATGTGATG
GGATTTCTGAGGCCATGATATAAATAGGATACAACTGATCCAGCAATCGCACTACTGAGTATCTACCCAAAGGAAAATAAATCAACATGTTTATTGCA
GTACTATTCACAACAGCAAAGATACAGAATCAGCGTTGAGTATCTATTAGTGGATGAATGGATAAAGAAAATATGGTATATATACACAATGGAATATT
GTTCAACCATAAAAAAACAAAACAAAATAATGTCACTTACAGCAACATGGATGGAACTGAAGGTCATTATCTTAAGTTAAATAAGCTAGGCACAAAAA
GACAAATATCATATATTCTCACTTATATGTGGGAGCTAAAAATTTGAACACATGGAGGTAGAGAATGGAAAATAGATAAGAGAGACTGGGAAGGGTGA
GTGGGGGAAAGGGGAAGGATGAAGAGAAGTGAGTTAAAGGGTACAAACATACATTAAGATAGGAGTAGATTCAATGTTTGATAGCAGAGCAGGATGAC
TATACCTAACAAAAATGTATTGTACCTGGTGATGAACATCCTGAAGACCCTGGCTTAATACTATGCATAATATACATGTAACATAATTCCTCATGTAA
TCTGTAAATTTGCACAAATAAAAAGAGGGAAAAAAAAAAGCTACAGCTTCTTCCTGGCTCTCTCTTTGTCTTGGGATGCTTGTTGTTGGAACTAAGTC
ATACGAAGAGGCCACATGTGGGTATTCTCTTCAAAAACCTGAGCTAAGCTGCAGCCAACAGCCAGTATTAACCACAGTATGAGTGAGAAAGCTTTCAG
GTAATTCCAGCCCCAGGTTGTCGAGTATCCTGCTGAGGCCCCAGAAGTTGCGGAGCAGAGACAAGCCAGCCCCACTGTGCCCTGTCTGAATTTATGGT
CCACAGAACCTGTGATAGATAATAAGTGATTATTGCCTTAAGCCACTAAGTTTTGGGGTAATTTGTTACATGGCAGTAGATAATTCATATAATTTCTA
GAAACTTATAGGGAAATAAAGATTTGCTCAAAGACATATATATATATATATATGTGATATATAAATGATCCATGTATATATATATGATATGGGTGATA
TATATAAAATAAATATGGGTGATATATATATATACACACACACACACACACACACACACATACATATATATATGTTCTCTGCTGTATCATTTATTTAT
GTTTTCTTTTTTTTAGAGGCAGAATCTCACTCTGTCACCCAGGCTAGAGTACAGTGGCTCAGTCATAGGTCACTGCAGCCTTGAACTCCTGGGCTCAA
GCGTTCCTCCTGCCATAGCCTCCCAAAGCACTGGGATTATAGGTGTGAGCCACGATGCCTGGCCCTGCAGTATAATTTACAATTTTTTGAAATAGTCT
AGCTATCCAACAATAAGAAAATGTTTAAATAAATTATCATAAGCACACTATTTTCCTAAGTAAAATTTCTACATTTTCTATATTCAAAAACAAAGTTT
AAAGAATTGGTATATAGTCAATTATGTCATAATGATAAACCTAATTGAATTATATATTTACATAAATAAAGATCATTAAGAACAAAAAAGGCTTTACA
TCTGATAAAGGACATGTACCCAGAACATACAAGAACTTTCGTCAGTCAATAAGAAAAGATACTAAATGTTTTAAATGGACAAAAGAATTGAACAAGCA
CATCCCCAAAGAAGATATTCAAATGGCCCTTAAGCAAGTAGAAAGGTCCTCAACATCACTACTCATCAGGGAAATGCAAAGTAGAGAACAAAGAGATA
CCGGTAGACGTTCACTGTACTGGAATAAAATTTAGCAGATGAGCAATATTGAGTGTTGACAAGGATATGAAGTAACTGGAACCCTTACACCTTGATGG
TGTGAGGGTAAATGGTATCACTTTGTGAACTGTTGGGTAAAACCAATGTTGACCCCATAAACTTTCAGTCCCATTCTTGGGTATATACCTAAGAGAAA
AGAGTGCTTATGTCCACCAAAGCACACATAGAATTATATTCATAGCAACTGTATTCATAATGGCTCAAATTGGAAAGCTGCTCTAGTCATACATTGTC
TGCCCTTACCTTTATTAACGCAGTAGTGCTTAGCATGTAGCTACCTGTGTTTAGTTTAATATGTATTATTAATGAAAATTTATTTAGGGGATTTCACC
CCCAATTTTTTAACTTTATGGTTTCAAATTTCTTAAAACCTTTCTCTTTACATCAGTTTGGTTTTGGAAGACTAAAAGAAAGAGCTTGTTGCACAGAT
GGCCCAAAGCACAAATTATTAAAATGCTAACTTTTATCTACATCCAGCTCCAAATATTCCCTTACTCCTTGATCATTCTTTTTTTTTTTTTTTTTTGA
GACTGAGTCTGTGTCACCTAGGCTGGAGTGATAGATGTATATATGGTCCCCTATTCTAAGGTAATACAGTCATGCATCCGAGAAATGTGTCATGAGAT
GACTTTGTCATTGTGAGAACACCATGGAATGTACTTACAGAAACCTGGATGGTGTAACTGCTATACACCTAGATGGTGTAACTACTACTATGCATAGT
CTATATGATATAGCCTATTACTTCTAGGCCACAAACCTGTACAGCAGGTTACTGTACTGGATACTGTAGGCAGTTATAACACAGTGCTAAGTATTTGA
GTATCTGAACATATCTAAACATAGAGAAGGTACAATAAAAACACAGTATAAAAGGCAAAAACTGGTACACCTGTATAGGGCACTTAACCATGAATGGG
GCTTACAGGACTGGAAGTTGCTCTGGGTGAGTCGGAGGTGAATGATGAGTGAATGTGAGGGCCTAGGACATTATTGTACACTACTGGAGACTTTATAA
ACTTTATAAACACTGAACACTTAGGCTACACTAAATTTAAAACAAATTTTTCTTGGCTGGGCATGGTGGCTCACTCCTGTAATCCCAGCACTTTGGGA
TGCTGAGGTCGGCAGATCACTTGAGTCCAGGAGTTCAAGACCAGTCTGGGGAGCGTGGCAAAACCCCATCTCTACAAAAAATACAAAAATTAGCCAGG
CATGGTGGCGCACACCTGTAGTCCCAGCTGAGGACTGGGAGGCTGAGGTGAGAGGATCATTTGAGCCAGGAGGTTGAAGGTGCAGTGAGCCATGGTAG
TGCCATTGCACTCCCATCTGGGCAATAAAGCGAGATCCTATCTCAAAAACAAATTTTTTTTCTTTAATAATATATTAAACTTAGCTTACTGTTAAGTT
TTTACTTTATAAATGTTTCAGTTTTTAAAAACTTTTGGACTTTTTTGTAATAACACCTAGCTTAAAAGACAAGTACATTGTATAGCTATAGAAAATAA
TTTTTCTTTATATCTTTATAGGCTTTTTCTATTTAAAAAGTTTAAATTTTTTTTTAACTTTTAGAATATTTTGGTAAAAATGAACACACACACACACA
CACACACACACACACACACACACACACACTGAGCCTAGGTCTACACAGGGTCAGGATCATGAATATCCCTGGCTTGCCCCCCTACATCTTTATCCACT
GGCACACCTTCAGGAGCAGTAACAGGCATGGAGCTGTTATCTCCTAGGATAACAATACCTTCTTCTGGAAGTCCTCCTGAAGGACCTGCCTGAGGCTC
TTGAGGAGATGTCACTCTTTTCAGAAATTTGCCCATGGTGTTTTGCTTGGTTTATTTCTATTTTTCATTGTAGATTTGTTTGTAAGCAGTTAATGCAG
CATGAACATTCCTGTGTATTAATGAAAACCTTTCTGTTTTTGGGGGTCCATGTTTTCACACTTCCTAAGGAGTTTGTTGAGGTGTGCAAAAGCTTCTC
CCAAACCCTTCACTGTGAGTTTTGTTGGGGGTTCTTTTTCTTTCTCCAGCAAACTTCCTTCTCTCTTGCTGCTTCTTCAGCTTTGCATTCCTATTCCA
GTTCCCACTCATTAGTCAATTCCTGCGATGTTATTGTGGCTATAACGTTACTAGATGACAGGAGTTTTTCAGCTCCATTTAACTACAGTCCATTGTTG
ACCAAAACATCATTTTGTGGCACACAACTGTATTTAGCTATTTTAGTGGTTGGGTAGCTGCTATCGTTTTTTCAGTGAAATTTGAGTAGTTAAAATGA
GTCAAGTAGTAGCTGAAAGCATGACGAAACTTATAATAGGACTTTCTTTACTTGTAAATGATACAATATGGTATGGCTAGATTAAGTCTTTTAAAAAT
ACTCATAGATTTCAACTTGGTTCCATAAAGCTGGAAGTGGAAACATTGGACATATATCAGAAATTCAATTGAACGCCTCCTTTTAAATTTATGGTTTA
CAGACTTGTGGCTCTTAGATCCCAGGGGATCTAAAGTAGACTGTGTTATAAAGTAGGCACATAACAAGACAACAGCCAATTTAAAAATATTAAATGAA
CTTGCTAATTCTCCATGATATTATATATATAAACTTTGTACCTAATGTAAACTGAAGGTAGTTATTAGCATTTCCAGGTTAGAAAACATAACTGGACT
TCCCTTATCCACTTTTTTTTTTTTTGAAACGGAGTCTTGCTCTGTCGCCCAGGCTGAAGTGCAGTGTCACAATCTCGGCTCACTGCAACCTCCATCTC
CCAGATTCAAGCAATTCTTCTGCCTCAGCCTCCCAGGTAGCTGGGATTACAGGTGTGTGCCATCACACCCGGCTAATTTTTGTATTTTTAGTAAAGAC
AGGGTTTCACCATGTTGGCCAGGCTGGTTTTGAACTCCCGACCTCAGGTGATCCACCTACCTTGGCCTCCCAAGGTGCTGGGATTACAAGCGTGAGCC
ACACCACACCCGGCCCCTTATCCATTCTTTTATGCTCTGTTACTACAGCAAATTATTCTCAGACATAGCTCCATCTTGGTAGTAGTGTTCTTGTTATT
ATCAGTAACAAAGTACATTAAGCTACTGCCTTTTGTTTTATTCTGTTTTGTTTTGTTTTATTTTGAGACAGTGTCTTGCTCTGTTGCCCAGGCTGGAG
TTCAGTGGCGCGATCTCAGCTCACTGCAACCTCTGCCTCCCACGTTCAAGCGATTCTTCTGCCTCAGCTTCCCGAGTAGCTGGGATTATGGGCACTTG
TCACTACACCTGGCTAATTTTTGTATTTTTAGTAGAGGTGGAGTTTTGCCATGTTGCCCAGGCTGGTCTTGAACTCCTGACCTCAAGTGATCTGCCTG
CCTCAGCCTCCCAAAGTGCTGGGATTACAGGCGTGAGCCATGGTGCCCGGCCGAGACTTCTAAAACATAGGTGTCATTAGGACAGTTCTATAGGAGAA
ATAAATGCCATTTTAGTATTATGACTACTTTTCTTTCATTTTCTTTAACCATAGTTTCATTTTTAACACCTGTTCTATAGATAGAAAAACAAAGATAC
TCATGTCATCTAGTGTCTTGTCTCATTTATAACTTGTTCCCTGAATCAGCTTCAAGTCTTTCATGTTTATGTGTAAAACATAGGGCTGGGCGCAGTGG
CTCACTCCTGTAATCCCAGCACTTTGGGAGGCCGAGGCAGGTGGATCACCTGAGGTCAGGAGTTCGAGAGCAGGCTGGCCAACATGGTGAAACCCTGT
GTCTACTAAAAATGCAAAAATTAGCCAGGTGTGGTGGCATGCTTCTGTAATCCCAGCTACTCAGAAGGCTGAGACAGAAGAATCGCTTGAACTCAGGA
GGCGGAGGTTGCAGTGAGCTGAGATCGTGTCACTGCATTCCAGCCTGGGTTGACAGAGCAAGCCTCCATCTCAAAAAAACAAACAACAAAAACCATTA
TAATTTATGCACACACAAATATTTAAAATGACTGTCACCTTTTTATACTTAGAATTGATCATTTATGATACATAGTATCTTAGAATTTTTTCCCCACG
TACTGGTGCTGTGGATGTGAAATCATGGTGATTTATTAGGTTTAATTTGTCATGTAAAAGAATTGTGTTCTGTTTGTTCTCTATACATTTAAATATTT
TAAATTATTATTATTATTTTTTTGTAGCTGTCCCCTTTCCTCCAACCCAACGGCTTACTTTCAAGGAAGTATTTGAGAATGGGAAACCTAAAGTTGAT
GTTTTAAAAAACCATTTGGTAAAGGAAGGACGACTGGAAGAGGAAGTAGCCTTAAAGATAATCAATGATGGGGCTGCCATCCTGAGGCAAGAGAAGAC
TATGATAGAAGTAGATGCTCCAATCACAGGTATAAAAAGTCTTTGCATGATACTTTTTTACAGTATAGATTTGCATGAGCAGTTTTGAGAAATAATTA
CAAATAACCAGCTAAAAAGTGGTGTGGTAATTTTTCTAGAAATTATGAGACAGTCAGGATTGGTTAGGATATTTGTTGTTAATTGAAGAAATACAATT
TTAAGTGTCTCATATTTCCAGTAGAACTATTTAGTATGAGTAGATTGACTACAGTTTTACAGCAGTCCTGTCAAAAGCTGGAGTGATTTAAGTTAGAA
GTTAAACTGTGATCCCTTTTGGTGTAATGCCCTTCGTCTTCTAAGGTATAATTTCTCACATCACTTTATGCTTTTTTTCCTAGTATGTGGTGATATTC
ATGGACAATTCTTTGACCTAATGAAGTTATTTGAAGTTGGAGGATCACCTAGTAACACACGCTACCTCTTTCTGGGTGACTATGTGGACAGAGGCTAT
TTCAGTATAGAGGTAAAAATTAAACTGGATATGTTGGGACTATTATATTGTCTTTCAAAAAAGATGATTTCCATTCTTCAGTAGAAGAAATTAAAATG
AGAACTCTGGCAGAATTTATAAACTTTGTTACTTTCAAAATTAAAGTAGTAGAAATGTTTTAATTGATAGAACAGTTATTCCTTTTTTAAAACCAGTC
GAAAATCTGACAAATAGGCTATATATTAATATTAACCCAATTTTACAAATAGTTAAATGTTCACCAACTTTTCCTCTTGAAGATAGTATACAGGATTG
TATAAAAATGACATAAATTGCTGTTCACCCTAAATGGGGTTTAGATTAGCTGACCTTAATTATAAGACCATAAGCTGCTGAGCTACAATTAACACTTA
GATTAACTGCCAGTGCTAAATAAACACTAAGCTTTTTGTAGGAATAATGTCTGGTACTGGTACACATAGTAAGCACTTAATAAATGTTCACTGAAGGG
GAAAAATGAGTTGTAGTTAATTAAAACTTATACTTTCCTTTTATATATTTTGAAAATGGAAAGATAGGAACATGTTTAGAATTAAATAGAAGCTTTAA
TGTGTAAAAGATAAAACTACTTTAGAACTTTATGCAAGGACTTGAGTACAGTGGAACCACAGCACATTCCTAGTTAATGTTCATTGATTCAACTCTAC
ATGCTGGCAAATAAAAGAGAAAAAAACTTTTGAAATAGCGTGGGTAATAGGATTTGTAATGTGACCAAGAATAAGCATGAGCTGAGAAATTGAGTCTG
CTTTCTGTCTTGCTGTCTATAGTCCTGCCTGCCCTCATTGTGTGAGAATCTCACTGTACTAAGTGTGTTTCTGGTGGTTAAAGGTTTATTGTCTTGCA
GCCTCACTCCTAAATGCAAGTCAGCTCTCTCACCTGATGCCCAAGCAAAGGAGCAGCAGTTGCTCTATTGCTGTAGAAAAAATAAGCAAAAAAATGGA
TTTTTAGTTTTCTGTTAGATAGGCAATCTAAATTCATGAATCAGAAAAGTTCAATGTTGGCCAGGCACGGTGGCTCACGCCTGTAATCCCAGCACTTT
GGGAGGCCGAGGCAGGCAGATTACGAAGTCAGGAGATCAAGACCATCCTTGGTAACATGGTGAAACGCCGTCTCTACTAAAAATACAAAAAATTAGCC
GGGTGTGTAGGCAGGCACCTGTAGTCCCAGCTACTTGGGAGGCTGAGGCAGGAGAATGGCGTGAACCTGGGAGACGGAGCTTGCAGTGAGCTGAGATC
ACGCCTCTGCACTCCAGCCTGGGCGACAGAGCGAGACTCTGTCTCAAAAAAAAAAAAAAAAAAGTTCAATGTTAGTTGCCAGTGCACAAAATTCATTT
AGTGTCAAATGTGAGGACAAAAGTTAAAGGTATAAACTGACAAAATAGTAAAGCCCATTTTTCCTTCTGCTCCGTAAATGTCCAAGGTCTCTAAATAG
GTACTGGGAATAGGCGGAGATTATCCTTGCAGACTGTATCATTGAGGAGCTTCCCTGTTGCCACTCATATCATGCATGTTTCAGTAGACATAAGTCAA
ATGTATGGATGTGGGGCTGCGCGCAATGGCTCATGCCTGTAATCCCAGCACTTTGGGAGGCCGAGGTGGGTGGATCACCTGAGGTCAGGAGTTCGAGA
CCAGCCTGGCCAACATGGTGAAACCCCATCTCTACTAAAAATACGAAAGTTTACCAGGCATGGTGGTGGGCGCCTGTAATCCCAGCTACTTGAGAGGC
TGAGGCAGGATAATCACTTGAACCTGGGAGGCAGAGGTTGCAGTGGGCTATTGTGCCGCTACACTCCAGCCTGGGTGACAGAGCAAGACTCTGTCTCA
AAAAAAAAAAAGAAAGAAAGAAAGAAAGTATGGATGTTAGAAAGACACATTGAGTTCAGTTTATTTCTTTGTGTTTTTGCTAGCATGAGAAGGTTTCA
GACGGTAAAGATTAGAAGAATACAAAAATTACTGAATTTCTAATTAGGCATTTTATTTTATTTTATTTTTTTAGAGATGGGGTCCTGCCATGTTGCCT
AGGCTGGAGTGCAGTGGCTATACACAGGTGCGATCATTTCGTACCTCAGCCTCCAACTCCTGGATTCAAGCCATCCTCCTGCCTCAGCTTCCTGAGTA
TATGGAACTATAGATGCACACCACTGTGCCTGGCTAGTCATTGTATTTAATAAAACAACTTTTAAAATATTTATTTATTTATTTATTTATTTATTTTT
TGAGACAGAGTTTCGCTTTTGTTGCCCAGGCTAGAGTGCAATGGCACGATCTCGGCCCACCGCAACGTCCGCCTCCCAGGTTCAAATGATTCTTCTGC
CTCAGCCTCCCAAGTAGTTGGGATTACAGGCACCTGCCACCACGTCTGGCTAATTTTTTGTATTTTTAGTAGAGACGGGGTTTCACCATCTTGGCCAG
GCTGGTCTTGAACTGCTGACCTCGTGGTCCACCCACCTTGGCCTCCCAAAGTGCTAGGATTACAGGTGTGAGCCACTGCGCCTGGCAATAAAACAACT
TTTTTTTTTGAGATGGAGTCTTGCTCTGTTGCCCTGGCTGGAGTGCAGTGGCACGATCTTGGCTCAGTGCAACCTATGCCTCCTGGGTTTTAAGCAGT
TCTCTGCCTCTGCCTCCTGAGTAGCTGGGATTACAGGTGCTTGCCACCACACCCGGCTAATTTCTGTATTTTTAGTAGAGATGGGGTTTCACCATCTT
GGCCAGGCTGGTCTTGGACTCCTGAACTCGTTATCCACCCACCTTGGCCTTCCAAAGTGTTGGGATTACAGGCATGAGCCACCGCGCCCAGCCTAAAA
CAACTTTTTAGTGATAAATTATTATGCCAAATTATCTTCTGATTAACTCGTATAACTTAAAATAGCTTTAAAATAGTTTTAATTCATTAATACATTCA
ATAAAGCACATAGAAAATATCAACAATGATTTTGTGCACAGACATCATGTTCAGAATATTTTGTGTTATGCATTTTTTTCCTCACTCATCAATAAAGT
TTCTTGAGTGCCAACTTTATGGGCAGGCTGTGAGAATAAAATAAAGAATAAAATTTATGCTCTGTAGGAGCTTCCAGTCTAATAGAAAAGATGCACTT
ACGAATAGACTTTTGTTATAATATAAAATGTGCTTCTTGAATTAACAGCGTTTTTCATATTAGGACTTAGCACAGTGCCTAACACATAGTAGGCACAT
AGTTAATATTTGTTGAATGAATGAATGTTAATAAATCATAGTTTGTATTTAGCTTTATTCTGTTAATTAGATGTTTGCTAACTTAAAGGGTTTTTTCT
GCAATAACTTCAGAGATAAATGAATCAAAACTTTAAGGTGACAATGACATTTTATACCAGTGTTAAAACCAAAGTATGAGATCTATCGAGTATTCATT
GGAAGTCACTTATTTATAAACCACTAAGATTATAAACCTCTATAGGCTATAGCTTACTCTAACATGTTGCAAGTATACACTTGAAAAATAATTTTTTA
ATAAATATTCTTGATAGCCATGAGTGTGATGTTTTTGTTTAATAGATGAAAATAAGGTCATGGAATTCTTATTAAAATTTTTTGTTATAAATAATTTA
TTTTTAATAGAAAAGTTGGGAAATGCAGAATCTAGAAAACAAAAACTTTCTACAAATCAGAGATAATCACTGTTAATGTTTTATTATACTTTCTTGCA
GCCTTTTTTCTTCTTTTACATTTGTATATATTTTGTTTTCAAGAAAATTGAGAGCATTTTGTACGCTCCTTTTCTGATCTTCTTTTTCCACTCAAGAT
TGTATGGAAAGCATTTTTCTGTATTGTTTTCTCCAGAAGTGTAGTGTCAAAGTCAAATAAAATAGAGACAAATCTCTGAAATTAAAACATTTTATCTG
AGAAGAAAAAAGACCAGCCTGGCGAACATGGTGAAACCTCGTCTGTACTAAAAATACAAAAATTAGCCAGGTGTGGTGGTGGGCGCCTGTAATCTCAG
CTACTTGGGAGGCTGAGGCGGGAGAATGGCTTGAGCCTGGGAGGTGGAGGTTGCAGTGAGCCCAGATCGCCCCACTGCACTCCAGCCTGGGTGACTTA
GCAAGACTCTGTCTCAAAAAAAAAAAAAAAAAGAAAAGAAAAGAAAAAATGCAATTTGGGGCATACACACAGACTGAGCGGGCTTTGGTATGTCTGAA
GAAAAGAGAAGGTTAGGTTTTATGAGAAAGAAAAATGTTATGTATTGCTGTTTGAGAAAGTTCATTGACACTAGTAAGGTTCTGGAGGGCTGACAAGT
TTTACTTAATCTCATGTAATCAATTTATATTCTGCTTGATCTCCGTTAATAGTTTCATGAGTCCGTCAGTTTCTTTATTAGAGTTGTGGAAATTTTTA
TTTAGCCTATTGCACTTAATGTTATCAGCAACCTATATTTAAGAGTACTTGTTGAAGTCTTTTCCATGAATCTGATTGTGCATGTTTTTAGAGACAAA
ACAGTAACTGTGGATGACAAAAGCTTAGAACAGCCATGGTTAAAAATCTGATGAAAGTTTACGATTGATAAGGAATTTTGTTATTTCTATTATAGATA
GCATTTTAGGATACAATAATCAGAATTAAGACTGAGTTATGAGTTATACAATTTTTATGAGTTATACAATTTTTGAAACATTTATGTTAACAACATAC
CCCTAAATGCAACTAAAATAAGATCTAGTATCACTTACCATTTAACAGTTTCTTATATAATTTACTAAATGAGCCTGATCATTTCATATCTCTGTAAG
ATGAGAGATATAGATCTTTTGAGGCTTTCCAGGGGCTGAACTGGAAAATCCCAAAGTTAATTTAATTTAATTTTATTTTTTTGAGACGGAGTCTCGCT
CTGTCACCCAGGCTGGAGTGCAGTGGTGCAATCTCAGCTCACTGCATCCTCCGCCTCCCAGGTTCAAGTGATTCTCCTGCCTCAGCCTCCCAAGTAGC
TGGGATTACAGCCCCCCACCACCATGCTCAGTTAATATTTGTGTTTTTAATAGAGATGGAGTTTCACCATATTGGCCAGGCTGGTCTCCAACTCCTGA
TCTCAGGTGATCCACCTGCCTCAGCCTCCCAAAGTGCCAGGATTACAGGTGTGAGCCACTGCAGCCAGCCCCAAAGTTAATTTTAGGTCAAAAGTACT
TAATTGAGGCTGGTCACAGTGGCTTAAGCCTGTAATCCCACTGCTTTGGGAGGCCTAGATGGGAGGCTAACATGTGTCCAGGAGTTCAAGACCAGCCT
GAGCAAGACCTTGTTTCTATAAAAATAAAAAACAAAAAAAGACTTAATTGAGGATTTGATTTTGGGGACGTTTGTCAAAGATGTCAAAAGGCTCAGAA
CATCTGATCAAAACAGAATCACACAGGTCACTATAAAATAATGGTCATTTGGCTGTGCGCAGTGGCTCATGCCTGTAATCCCACACTTGGGGAGGCCG
AGGCAGGTGGATCACCTGAGGTCAGGTGTTTGAGTCCAGCTTGGCCAACATGGTGAAACCCCGTCTGTACTAAAAATACAAAAAGTTAGCCGGGCGTG
GTGGTGGGTGCCTGTAATTCCAGCTACTTGGGAGGCTGAGGCAGGAGAATCACTTGAACCTGGGAGGCAGAGGTTGCAGTGAGCCAAGATCGCGCCAC
TGCACTGCAGCCTGGGCAACAAGAGCGAAACTCTGTCTCAAGAAAAATAAATAATAATAATAATAATAATAATAATGATAATATTCATTCAGCCAGTG
ATAAAAGATTTCCAAAGTAATACAGAAAGTTACATGGATGTAAAAACGTTAGTTTCCTTAAAGTTTAGTTTTCCTAAGTTATAAGATACCTAATGAGG
GCAACACAGAAATTATCTTGATGAAATGTGGAAGTTTTGTGTTCTTTTTTTTTTTTTAATGTTTTAAATTCTGGTATACATGTACAGAACATGCAGGT
TTGTTACATAGGTATACATGTGCCATGGTGGTTTGCTGCATCCATCAACCTGTCATCTACATTAGGTACTTCTCCTAATGCTATCCCTCCCCTAGCCC
CCCACCTACCAATAGGCCCCAGTGTATGATGTTCCCCTCCCTGTGTTCTCATTGTTCAACTCCCACTTATGAGTGAGAACATGTGGTGTTTGGTTTTC
TGTTCCTGTGTTAGTTTGTTGAGAGTGATGGTTTCCAGCTTCATCCATGTCCCTGCAAAGACATGAACTCATCCTTTTTTTTGGCTCCATAGTATTCC
ATGGTGTATATGTGCCACATTTTCTCTATCCAGTCTATCACTGATGGACATTTGGGTTGGTTCCAAGTCTTTACTATTGTGAATAGTGCTGCAGTAGA
CATACGTGTGCATGTGTCTTTATAGTAGAATGATTTATAATCCTTTGAGTATATACGCGATAATGGGATTGCTGGGTCAGATGGTATTTCTGGTTCTA
ATCCTTGAGGAATCACCCCACTGTCTTCCACAGTGGTTGAACTAATTTACACTCCCACCAACAGTGTAAAAGCATTCCTATTTCTCCACATCCTCTCC
AGCATCTGTTGTTTCCTGACTTTTTAATGATCGCCATTCTAACTGGCATGAGAGGGTATCTCATTGTGGTTTTGATTTGCATTTCTCTAATGACCAGT
GATGATGAGCTTTTTTTCATATATTTGTTGGCCGCATAAATGTCTTCTTTTGAGAAGTGTCTGTTCATATCCTTTGCCCACTTTTTGATGGGGTTGTT
TGTTTTTTTCTTGTAAATTTGTTTAAGTTCATTATAGATTCTGGATATTAGCCCTTTGTCAGATGGATAGATTGCAAAAATTTTCTCCCATTCTGTAG
GTTACCTGTTCACTCTGATGATAGGTTCTTTTGCAGTGCAGAAGCTCTTTAGCTTAATTAGATTCCATTTGTCAATATTGGCTTTTGTTGCCATTGCT
TTTGGTGTTTTAGTCTTGAAGTCTTTGCCCATGCCTATGTCCTGAATGAAGCATATAAAAGTTTTGTTTTTTAAACAGGCTACCAAAAAGGTGAAGAA
AAACCTTCTGTAGTATGATTGTTTCTTCTTATGTGATGTTCACTTACATAACCTGGAAATCAAACCGGATGAAAAGAGTACTTGAATTAATCAGACAG
AGGAAGAATGTGCCCAAGATTATGAATGTTTCAATTCAGCTACGTCAAGAAAAGCCAAGATACAGTAGAGGAAAACATAGCTTTTTTAGGCTTTTAAG
ATAAACCTCTTAGTGTCAGATCATAACAGTCGAGTTAGAATAGGAGAAAAAAAGTTATAGGAGCAGATGAAAAAGTTGGAGAGAGTCACCTTCTAGCC
AAGCAAAAAGATACGCTTTTTCAAGTGGAGAAAGAACAGAAGGGAATAATATATGACTTGCAATCATGTACAGTGAAGGACAGCAGAATTTGAACTTC
TGAGGTATACATTTGAGAAGTTTCAAAAAGAAACAGATTTCAGAATTAAGTATCAAAACCTCTTGCAGTTTTACTAAGAGAATGTCAATACTTTAAGA
AAAGCCCTGTTCTACAATAGGAGACCAAATTTTATAGTTTTGTATTAGTGTCTTTTTACTATCAAAGCTCAATCTTTGGGAAGACCACATGTCATTTT
ATTTTTTTATTTTTCAATTTTTATTTATTTATTTATTTTGACACGGAGTCTCGCTGTGTTGCCCAGGCTGGAGTGCAGTGGCGCAATCTCGACTCACT
GCAGGCTCCGCCTCCAGGGTTCACGCCATTCTCCTGCCTCAGCCTCCTGAGTAGCTGGGACTACAGGCATGTGCCGCTACGCCCGGCTAATTTTTTGT
ATTTTTAGTAGAGACTGTGTTTCACAGTGTTAGCCAGGATGGTCACGATCTCCTGACCTTGTGATCTGCCTGCCTGGGCCTCCCAAAGTGCTGGGATT
ATAGGTGTGAGCCACTGCGCCCAGTCCCATATGTCATTTTATTTTAATTATAGCCAGCTTGATCACATACAAAATTCCATTTATAAATTCCGTTTTCA
CAAACCCTATTACAATTTCACTCAGACCATTAATGACATGCTTGGACTTTCTGCTTTGTCCTGTATTTCCTTTCTTAAATAACCATTTTATTTTAGGA
TGAAAATTTGCCACATAAGATTCTTTGTCATACAAAATTATTCTATTTCTCAATAAAGGACACATTTTAATGCCTATCTTGTAATTAAAAAAAATTTT
TTTAAGAGACAGAGTCTTGCTCTGTCACCCAGACTGGAGTGCAGTGGTGCAATCATAGCTCACTATAACCCTGAACTCCTGGGCTCAAGCAGTCCTCC
TGCCTCAGCCACCCCAATTAGCTAGGACTACAGGTGTGTGCTACCACACTTGGCTAATTTTTTTTTTTTTAATTTTTTTAGAGTTGGGTCTCACTGTG
TTGTTCAGGCTGGCCTTGAACTTCTGGCCTCAGGATACCCTCCAACCTCAGCCTCCCAAAGTGCTAGGATTACAGGCATGTGCTACCACAGCCAGCTT
GTATCAACAATTTTAAAAACAATTAATTTATCAAAGATTTACTTAAGTCACAAGAACTGAAAAAACATTTGGGTTAATTACTACATATTTTATATGAG
CACTTGTTTACTTAAACTAATCAGAATAAATTTTTTTAGGGATTTCTGGCTGACTATATGAGATTTTATTATGTAGACACAGCATACAACATAGTATA
TATATATAATATAAGCATATCTACACAGATATACATACTTATAAATAAATTCTTATGGCTTTTGTTTTAGAGTTTTGGTCATGAAATAGTAAAACACT
CACCAATTTATAAAAAACAGTAGGATCCAGATTATCTTTCTGACAAAATGGAATAAGGCCAAACTTAATGTTCTTTTTTAAAACAGGCAATATTATAA
GGGTTGTGAACTAAATTTTGAGTAAAGTAGTTTGAGTCAGTTACCAATAAACTGGATTCGACACAGAATAGTTAATTGTGGAAATATGACTGAATTAT
CTTCTAACAAGGTCTGTTCAGTATTTTATTGGTCTCAATTCTTTATCCTTGAAGATAAAAGTGTTGCCATTCCTTTTACTGTGGGCAGGCTATCTTTA
CATGGGAATTTCATTTTTGCAAAGGAAAGAGCATGAAGGTCAAAATGATTTTCTTTAATATCTGCTGGTTTTAAGTGTTTTACTTAAATGGTCAGTAT
GCCAGAATAGTTGAAGAGTTTTGAAGAAAGAGTTTAGAAAAAGATAAGAGGAGGCAGAGCGGGAGGGGAAAGCTCTAGAGAGGAAAGTGATTCAGCTA
GCTTTGAGGCAGTGTTTTGTAGTGAGTCATTCTTTGAGTCCTGAATCCTTTTTTTTAGCCTCAAAATATCAATGAGATACGCATATCTTATTTGGAGT
ATATTATTTTCTAACTGTGGTCTTTTAGTTTAAGGAACAACAGACTGTTTGAATGATCCTTACGATGTTTGAACATGTTACCGGCTGGAATCCCAGAA
AATATCTTGGCATGCCTTTGAATTTTGAGAGCCCATTTTATTGTGGGTGCTCTGGTTTAATCCCAAATACACAAAAACCAATTAAATATAAGCACTGA
ATTTGCAAAGGCCAAATAAACGCAAGCACAGATGGGAAAATAAAGTACCCACTTACCAAGAAGGACGATAAGAAGCCTTCTCCAACTGAAAGAAAAAA
GAGAGATCCCAAGGCCTCAACCCAAAGGGAAAAATCCCTTCCCTGAGACCCCACAGCCAGGGTCTACAAGGAGGAAAAGAGTCTTCCTTAATAGGAGA
TCCCTCAGCCAGATCAGACGGGAAGGGGAAAGACACCATCCCATCCAAATCCCAGATAAAACAGAACTCAACCAAAATCGGGAGTTCAGTCCAAGAAT
GACTCACTAAGGGAAAAAGAGGCAACTTGCAGAAGCCGTTAGAGGAGGGGGTGACTTTCAGAGGTCCCCAGTGTGGCTACCTTGTACTATAGTTCCAA
TGGCTGCTGATCTTCTCTGAGATGAGTCAGCCTTAGACTCATACTTGACCAAGTATGTCAAAGTCAAATGAAACACAGAGATGAATCTCTGAAATTAA
GATGTTTTATTTGTGAAGAAAGAATTGCAATTTAGAGCATGCATGCAGACAGAATGGTATGTCCGAAGAACAAAGAGAAGGTTAGAGGTTTTATGAAA
AAGCGAAATGTTATGTGTTGCTCTTTGAGAAAGTTCATTGGCACTAGTAAGGTTCTGGAGAGCTTGCCAGTTTTGATTGGTGAGTGATGGCAGTGGGT
AAAATTAGCCTTAGAATTTCAGCAGATCATTTCAGTAGCCATTAGATAAAACTGGTTTCCAGTAATAGCAGGCAGTTTCGGCAGACATGCTTGCAGAG
AATTACATTTTTGGGTCAATGTTATATGTCCTGAGTGCTTCCCCCCCGCCCTCTTGACTCTGTTTTAGTTGGGTATGACAGGAATAACCCAGTTCATA
TGATCAACTTTCACAGTAGTTAATGATATAAATACCTTAATTTATTTCACCATTCCTCTACTGTTGGAAAAATAGGTTTATAGCAGGTCATGCTATTG
CATACATTTTCCCTCTGTCTTCCTTACAAACAATATCTCTATTTTATTCAGATAGATGGGATTGGGGTTGCAGGGAGAAGCTTTGATCTCAGGGAAGG
AAGGCCCTTATCCCAGCCCCAGGGAATGAATTCTGGGTGGTCTGAACTACTCAAGCCAATCAATTTCCTGTCACTAATGATTGGTCTAGGGGTAAACA
CTGGATTTTCTTTTAACCAGTAATATATAAAGGGAACTCTGCTGGGTTTTTGTTTGTTTGTTTGCTTTTGGAAACACTCTTTCTTGATGAAAAGACTA
TTGCAAGGTTGAAGCTTTTGGACCCCCATTCTACCATTTCACTTCATATGTGGATGTGTGTACTGTAGCAGCCATGCTGTGACTATGAGGAGGTGACA
TGACTAAGGAGGAAAAGCAGACCTGTGAATCGCTGATCACAGAGGTACAGAGGGCTTGGTTTCTTGGTGAAGATGTTGAACTAGTACACCAAACCTGG
ACATCTCATCTCCAGACTTGATGTGTTTTTTGTTTTGTTTTTTTTTTTTTTTGGGACGGAGTCTCACTCTGTTGCCCAGGCTGGAGTACAGTGGCACC
ATCTCGGCTCACTGCAGCCTCTGCCTCCCAGGTTTAAGTGATCCTCCTGCCTCACCCTCCCGAGTAGCTGGGATTACAGGCACCCACCACCACGCCTG
GCTAATTTTTGTATTTTTAGTAGAGATGGGGTTTCACCATGTTGGCCAGGCTAGTCTCAAACTCCTGACCTCAGGAGATCTGCCTGCCTCAGCCACCC
AAAGTGCTGGGATTACAGGCATGAGCCACCGCGCCCAGCCCAGACTTTGTGTTTTATGAGGTATTTAAATATCTTACACAACAGCAAATTGGATTTGA
TATCACTTGTATTTAAATGGTACAAAGTTGTCTTCCAATTATTTCCACTCTGCACATTGCTAAAGTTAACATCTTTTTGAATATATCTGTGATGGCAT
TTCAGATACTTAGATCAGATTCTCACACATGAAGTAACTGATCAAAGAGTAGAACATTTTTAAGACTTCTGCCGCTGATAAAACAAAATAGTAAAGTT
TTATTTATCTTTGAGACTTTTGGTGTTCCCTCTTCATTTGTACCTATTACAAAATGCTGGCTGGGCGCCCTGGCTCACACCGGTAATCCTAGCACTTT
GGGAGGCCAAGGAGGGTGGGTCACCTGAGGTCAGGAGTTCGAGACCAGCCTGGATGACATGGTAAAACCCTGTCTCTACTAAAAATACAACAAATTAA
CCAGGCGTGGTGGCGTGTGCTTGTAGTCCCAGCCACTCAGGAGGCTGAGGCAGGAGAATTGTGTGAACCCTGGTGGCAGAGGTTGCAGTGAGCCTAGA
TTATGCCACTGCATTCTGGCCTAGGTGACTGAGTGAGACTCTGTCTAAAGAGAAAAAAAAAATAAAAACAAAGTGCTTCCTCTTCCTCTGTGAGAGAA
GAAGCTAAATTTTAGTTTCTTTAATTGATTTTGTGAGAAAAGAAAAGATTAAAGGGAACATTTTCAGCAAAGATATACTGTATTAAAAGCACAGAAGC
TGCCAAAGAGAAAAATGTTATCAGATGAGCAGGTGATATAAATGTTATTAGATGACCAATTGATGAACAGGTATTGACACATAGAAATGATTCTGGAG
GCAGAGTAAAAATTCTGTTCACACAGAAGTAAAAAGTAATTGAGTGGTTGAACAGATCTCTTGATAATCATGACCTTGTAATTGAATTATACAAGACA
ATTGTACAGTTGCATCCTGATGTATAAAATAAGGAAATAAAACTTGAAATTAGAGGCCAAGAAGCACATCACCAAATGACATTATACTATGGGTGGAT
TGTGCCTTTATATATCTCGTTACCTCAAAAAGGTGAGTTAGAAGGCACAAATGTATACCCAGATAAATCAAAAGATATATTGTGTTCTTGGTTGGGAA
AGGTAGGTATAGAAAAGAGAAGTGCATTCTAAATTTGTATATTGATTTAATATAATTTTTCCTTGGAATATTTTGTGAAAATTGTTATCTTGGTAATG
AAATTTAACCCCTCAAAAGAATAAAGCCACACAAGGTTATTTATATGTGTCTACATGTTTAAGCTTTATGATTATTTTGCAATGCCTGTTTCAAAATG
TGTATATGCTTTAATGCAACAATTCCATTTTTCAGAACCTATGCTTTAGATGCACTCATAATTACGTACAATGATGTTTGAATTGTTTGTCACAATGA
TTGAAAGTTTGTTTTGGCATTTGATAAACAGGCTTCAGGGATACAAAATACCCTGCAATGTGTAGGAGACACCTAAACAACAAAGAATGAATGATTCT
CAGATTGTCAACAGTGCCTGTGTTAGATTCTGGGGATCTAACAGGGAGATGCACAAGCAAAGTCCTTTACTGTATTTTCTAGTGAGAGGGACAGCATC
ATGTATTTGTTTACCATCCATGAGAACCAAATCAGAGGAAGGAGCTAAAGCGGGACAGGAGCAGCTAACTTAGATGGAGTGATCAGGGCAGGTTCTTT
TGAAGAGGCAATTGTTGAACAGAATGACCTGTGGGAACAAGCTGTGCATAGATCAGGGGAACTACATTCCAGGCAGAGGCACAACCAGTGTAAAAGCT
TACAGGCAGAAGCAAGTCTAGGGCAGTCCATGTATAGTAGTGAGGCTATCGAGGCTAGACAGAATAATGAGTAAGGGAGAGATGGAAGAGGTGCAGAG
AGTGGGGCAGATAGATGATGTCAGGCCTAGTAAACCATGTCAGTGCATTTGGGTTTTATTCTGAATGTGCTAGGAAGTTATTGGAGGAATTTTTTTTT
TTTGAGCAGGGGAGTGACGTATTCACATTTATGTTTTAAAACAATTACTTTGGCTGTTAGAAGGTCAGTTGACTTTTGGAGCCCAAAAGCAGGTGCAG
GGAGACCAGTGAGGATTTAATCTAGTAATATAGGTAAGAGATGTCAGTAATTAGATTAGGCATACTGAGAAACATTTGGATTCAGGATTTCAAAGTCT
TTCCTTTTTGAGGAGGTGGGAAAGTCTCAGTTGAATTGATCGAGCTCCTCAGGTCATGGCACATTTCTCATCAGTCTTTGCAAAGTCCCTCTTTTTTG
TTTTATTCTTTTTGTGCTTATCTGTGATCTCTGAGTTCCTTACTCCCTCTCCCCAGTATACACATGCTTCTACTGTATTTAATATATGATTCAGAATA
CATCTAATGTATATATGAATGAATGATTCTCAGATTGTCAACAGTGCCTGTGTTAGATTCTAGGGATCTAACATGGAGATGCACAAGCAAAGTCCTTT
ACTGTATTTTCTAGTGAGAGGTACAGCATATATATATATAACAAATATATATTATATATAATAGTGTATATAATATATAATATATAATAAGTATATAT
TACATATAATATACAATAAGTATATATTATATATAAGTATATATTATATATAAGTATATATTATATATTATATATAAGTATATATTATATATTATATA
TATAATAAGTATATATTATATATAAGTATATATTTTATATTATATATATAGTAAGTATATATTATATACATACATAACAAATAAATATATATATATTC
AAATAATTTGAGATGGAGTCTTGCTCTGTTTCCCAGGCTGGAGTGCAATGGCACGATCTCAGCTCACTGCAACCTCTGCCTCCCAGGTTCAAGCAATT
CTCCTGCCTCAGCCTCTCCAGTAACTGGGATTAGAGGCGCATGCTACCACGCCCAGCAATTTTTTTTTGTATTTTTAGTAGAGACGAGGTTTCACCAT
GTTAGCCAGGGTGGTCTGGATCTCCTGACCTCGTGATCTACCCACCTTGGCCTCCCAAAGTGCTGGGATTATAGGCGTGAGCTGCCGTGCTGAGCCAA
TACATCTAATATATTAATTTTTTAATGTATAGAGCTTTTACAGATAAAATATTTTAATTTTACATAAATGATATGGTGCTGTCCAGTGGTCTTGAAAT
GTTAACATACACCATTATTACCTGGAAAGCTTGTGCAAACAGAGGTTGCAGGACCCTGACCCTGTTTCTGACTCATCAGGCGTGGGGTGGAACCTGGC
AATCTGTATTCTGTCGGGTTCCCAGGTGATGCTGTTTACCACACTTTGAGATGAATGTGACCATACTTTGAGAACCACTGTTGTAGTGCTATTTATAC
TGTTGACTTTTTTCACTTACCACTTTTTTTATATTTATATATATTTTATTATACTTTAAGTTCTAGTGTACATGTGCACAACGTGCAGGTTTGTTACA
TATGTATACATGTGCCATGTTGGTGTGCTGCACCCATTAACTCGTCATTTACATTAGGTATATCTCCTAATGCTATGCCTCCCCCCTCCCCCCACCCC
ACAACAGGCCCCAGAGTGTGATGTTCCCCTTCCTGTGTCCAAGTGTTCTCATTGTTCAATTCCCACCTATGAGTGAGAACATGCGGTGGTTGGTTTTT
TTGTCCTTGCGATAGTTTGCTGAGAATGATGGTTTCCAGCTTCATCCATGTCCCTACAAAGGACATGAACTCATTATTTTTTATGGCTATGTAGTATT
CCATGGTGTATATGTGCCACATTTTCTTAATCCAGTCTATCATTGATGGACATTTGGATTGGTTCCAAGTCTTTGCTATTGCGAATAGTGCCGCAATA
AACATATGTGTGCATGTGTCTTTATAGCAGCATGACTTATAATCCTTTGGGTATATATCCAGTAATGGGATAAGATATATCCAGGTTACTACTATACG
TGCCTCTAGTTGATTGCTTTCTGCCTGCTGTATAGAATTTCACAGTATTTATGTATGACACTTTAGTTTTCTCTTTTGCTTGGTGGACACCTAGTTTA
CCTCCAGTTTCTTTCTAACATGAACACTGCTATGATGAACACCCTCATATAAATCTACCATGATTTAATGTCTCTAGGTTTTATGAACAAAATCGGAT
TGCCAGGAGAAAGGGTGTATGCATTCATACTTGAACTGAGCATTGGTTGCCAGATGGCTCTTCAGAATGGCTGTGCAAGTCTCTATTCCTACCAGCAG
CACTGAAGAATTCCTATTTCCTCACATCCCTCCTTCCAGAATATGATTTTATCTCATATTATAATACTTGCCAATATAATGGGTATAAAGTAGTATTC
TACTGTTTTAATTAATGTTTCTCTAATTACTGATGAAATTAAGCATCTCATATGCCAGCCATTCAGCTCTTGCACTCACTCTTAGGAGTTTCTTATGT
GACCTTTCAGAGATAGGTTAAACATATACAAGCATATTTGCAAATCTAATGAGAAAATAGTGTACTTGTCACTATACATTGCCCTTTAAGTGCTGCTT
TATCTGAGTCCCACAGATTTGGACATGTATTGCTTTCATTATCATTCAGTATTTTATATTTTAAGAATTCTCTTTAACCCTAAAATAATGAAGTAGTA
TGTTTTTTCTTTCCAGTCATATGGGATTTTTTTAGAGCTAGTATTTTGTTACTGATTATTGATTTGATGTAATTTGATTAAAGAAAATGATTGGAATA
ATACTCATTCTTTGAAATTTATTGAGACTCCTTGTGGTATAGTACAGAGTAGATTTTTGTGATTTTCCCATATATTCTTGGAAAGAACGTATATTCTC
TACTTGTTGAGCTATTCTTTATAACTTTGACTATTTTTGTCTGCTTGAACTATCAGTTACTTTCAGCATTGTGTCAAAATTTCCAACTACCATTACAG
TGTCATGCATCATTTAATAAGGAGGGTACGTTCTGAGAAATGGGTGCCGATTTTGCTGTTGTGAAATCATCATAGAGTGTACTTACATAAACCTAGGT
GGTATAGCCTGCTACACACCTAGGCTATATGGTATAGCCTGTTGCTCCCAGGCTACAAACCTGTACAGCATGTTATTGTGCTGAATACTGTAGGCAGT
TGTAACACAAGGGTAAGTATTGTGTGTCTAAACATATCTAGATGTAGAAAAGATACAGTAAAAAAAAAAAGAGAAAAAATGGAATACCTGTATAAAGT
ACTTATCATGAATGGATTGTACAGGACTGGAAGTTGCTCTGGGTGAATCAGTGAATGATGAGTGAATGTGAAGGCCTAAGACATTGCTGTATACTACC
AGATGCTTTATAAACACTGTACACTTGTGCTATACTAAATTTATTAAAAAGTATTTTTCCTTCTTCAATAATAAATTAGCCTTAGCTTATTGTAACTT
TTTACTTTATAAGATTTTTAATTTTGAGCTGCCATCTTGCGTCCCCATGTGTGTGCACCTAATCTCAGCTGGTGGTCCACCCGAGACCCCCAAGCACC
AACCCTAGCCCCCCACGTTGGCCCCTTATCTGCTCTGAGAAGATGAAACAAACAGTTATGAGCCAGGAAAGACTTGCCAAACTGCAGGCACAAGTGCG
CATTGGTGGGAAAGAAATGGTTCACAGAAAGAAGAAGGCGGTTCATAGAACAGCCACAGCAGATGATAAAAAACTTCAGTTCTCCTTAAAGAAGTTAG
AGGTAAACAATGTCTCTGGTATTGAAGAGGTGAATATGTTTACAAACCAAGGAACAGTGATCCACTTTAACAGCAGAGATGCCAGCAAACTCTTTCAC
CATTACAGGCCATGCTGAGACAAAGCAGCTGATGGAAATGCTACCCAGCATCTTAAACCAGCTTGGTGCACACTGTCTGACTAGTTTAAGGAGACTGG
CTGAAGCTCTGCCCAAACAGTCTGTGAATGGAAAAGCACCACTTGCTACTGGAGAGGATGACGATGAAGTTCCAGCTCTTGTGGAGAATTTTGATGAG
GCTTCCAAGATGGAGAATTTTGATGAGGCTTCCAAGAATGAGGCAAACTGAATTGAGTCAACTTCTGAAGATAAAACTTGAAGAAGTTATTGGGAGCT
GCTATTTTATATTATGACTGCTTTTTAAGAAATTTTTGTTTATGGATCTGATAAAATCTAGATCTCTAATATTTTTAAGCCCAAGCTCCTTGGACACT
GCAGCTCTTTTCAGTTTTTGCTTATATACAATTCATTCTTTGCAGCTAATTAAGCTGAAGAATCCTGGGAATCAAGTTTGAAACAAAGGTTAATAAAG
TTCTTTGCCTAGTATACCAAAAAAAAAAAAAGATTTTTAATTTTTTTAACTTTTTGACTCTTTTGTAATACCACTTAGCTTACAAGTGGTATTATGAG
TGAATGTGAAGGCCTAAGACTTATGCAGCTGTACAAAAATATTTTTTCCTTATATTCTTATTCTATAAGCTTTATTAAAACCTTTTTTTACTTTTAAA
ATTTTTTGGTAAGAATGAAGACACACACACACACATGCACGTGCGCACGCATCTAGGCCTACAGAGGGTCAGGATCATCAATATCACTGCCTTTCACC
TCCACATCTTGTCCCACTGGAAGGCCTTCAGGGGCAGTAACAGTCATGGAGCTGTCATGTCGTATAACAGTGCCTTCTGCTAGAATCCCTCCTGAAGG
ACCTGCCTGGGGATGTTTTACAATGAGCCTTTTTCTTTTGTAAATAGAAGGAACAAACTTGAAAGTAATTATTAAAAGTATGGTATAGTAAATACATA
AACCAGTACCATTTATTATTATCAGGTATTATGTACTGTACATAATTGTATGTGCTATACTTTTATATGACTGGCAGTGCAGTAGGTTTTTTTACATC
AGCATCACCACAAACATGTAAGTAATGCCTGCATGTAGACATTATGATGGCTACATCACTAGGGTGTAGGAATTTTTCAGTTCATTTTAATTTTATGG
GACCATTGTCCTGTATGTGGTGTGTTGTTGACTATAGCAGCATTATGTGGTGCATGACTATATTGATTTATCTGTTTCTCAACATAGGCCTCTGTATT
AGTTCTTTTTCATGCTGCTGATAAAGACATACCCAAAATTGGGAACAAAAAGAGGTTTAATTGGACTTAACAGATCCGCATGGCTGGGGAGGCCTCAG
AATCATGGCAGGAGACAAAAGGCAAAAGGTACTTCTTACATGGCGGTGGCAAGAATGAGGAAGAAGCAAAAGTGGAAACCCCTGATAAACCCATCAGA
TCTCATGAGACTTTTTCACTATTACAAGAATAGCACAGGAAAGACCGGCCCCCATGATTCAGTTACTGTCCCCTGGGTGTGGGAATTCTGGTAGCTAC
AATTCAAGTTGAGATTTGGCTGGGGACACTGCCAAACCATATCAGCCTCTCAGTTTTTGCTTGTGTTTTGAGGCTGTGAAATATATACAGAATATTTC
ATAAGTGGTGTATACAATTGATTATCATCATTCATGCGTTCTGTATTTGTGGATATGCCTATGTCTCCTTGCTAAAATTGATTTGTAAACTCAAAATC
AATACAGTGCTTTTGTGGTCACTGGTAGACATGTGGAGAGTGATCTACTACTTTGAGTCTCCCAATGCACATGTTGCCACCTCAGACCAAATAAGGCC
ACACTCTCCCTTCTTGTTTTAGCTCTCATGCTTTAAACAAGTATTCTTTTCATAGTCTATTTAGTGCCTGAATGCCTGCTATAGGTAACCAATCTCTT
CAGTTTCTGGTTTATCTTTCCTGCATTTTTTATGTACAAAGTAGATACATACATATGAATTTCATATATCTTCTTTATTATATAGAGGGTGGCATACT
ACAGCTACTCTTGTACTTTTTATTTTTTTTAAGAGACAAGAGTCTCACTCCGTCACCCAGGTTGGAGTGCAGTGGCACGATCATGATCATAGCTCACT
ACAGCCTCAAACTCCTCGCCTCAAGGGATCCTCCTGCCTCAGCCTCCTGAATAGCTGGGACTACAGGCACGTGACCACCACATCTGGCTAATTTTTAA
ATGTTTTGTAGAGATGGGGTCTTGCTGTATTGCCCAGGTTGGTCTTAAACTCCTGGTCTCAAGCGATCCTCCTGCCTTGGCCTCCTAAAGCACTGGGA
TTGCATTTGTGAGCCATTGTGCCCAGCCATACTTTGCTTTTTTCATCTAAAAATATATCTTGGAAATCACTCTAAATCAGTTTATAGAGATCTTTCTC
ATCCTTGTGCTTTATTGTGTGAGTGCATCATAGCTTAGCGTCTCTCCTGTGTATGGTCATTTAGGTTGTTCCTAATATTTTACTATTACACACATGCT
ACAGCCTTGTGCATATTTATTTTCGTATTGTTGGAAGTGTATCTTCAGGGTAGATTCCTAGAAGTGGGTCAAAAGTTAAATGAATGTGTAGTTTTGTC
AGTTTGGCCAAATTGTTTTTTTACTATGCAAATTATATTATTATTATTATTATTATTGTTATTATTTTTGGGATGGAGTTTCTCTCCAGGCTGGAGTG
CAATGGCGCTATCTCGGCTTACTTCAACCTCTACCTCCCAGGTTCAAGCGATTCTTCTGCCTCAGCCTCCTGAGTAGCTGGGATTACAGGCACCTGCC
ACCAGGCCTAGCAAATGTTTGTATTTTTAGTAGAGAAGAGGTTTTACCATGTTGGCCAGGCTGGTCTCAAGCTCCTGACTTCAGGTGATCTGCCTGCC
TCTGCCTCCCAAAGTGCTGGGATTGCAGGCGTGAGCCACCATGCCTGGCCTATTTTCATTTTTGTGTGGTCAAATTTACCCATCTTTTATTTTATTGC
CTCTGGATTTTTAGTCATTAGTTACACAGTTCTTACCTACACCAAATTTTCACCTACACCAGGCTTAAGAAGAACTCACTCATGTTTTCTTCTAGAAC
TTGTGTGGCTTTTTCTTTTTACATTTAGATTGCTCATCCTTTTGTTGTTTATTGTGCTTTGTGTGAGATGTGGATCTAACTTTATGTTTTTTTTTTTA
AATGGCAACCACTTGTCCCAGAGCCATTTATTTAAAAGTTCATCATTATTACTGTGATTTGAGATGCCACCTTTATTATTTACCAAATTTTTGTAGGT
ACTTTTATACTATATCTGTAGTATAAAATTCCACTGGTATGTTTATCTACATAAGGACCAGTACCACTGTTTTGATTATAGACATTTTATAGTATGTT
TTAACATCTGGTAGGGATAGTCTGCCCTCATAGTGTCTCTTTTTAGTGTTTTCCTGGTTATTCTTTCTTGCATGTTCATTTTTCCATGTTAATTTTAG
TGTCAACTTGTCTAACTCCATAAAATAGCTTGTTTGTATTTTTCTTGGAATTTCATGACTTATAAGTTAAATTAGGGAGAACTGTAATCTTTTTTTTT
TTTTTTTTTTTTTTTTTTTTTGAGACAGAGTCTCGCTCTGTCACCCAGGCTGGAGTGCAGCGGTATGATCTTGGCTCACTGCAACCTCTGCCTCCCAG
GTTCCAGTGATTCTTTTGCCTCAGCCTCCCAGGCAGCTGGGATTGCAGGCACTTGCCACTACGCCCAGCTAATTTTTGTATTTTTAGTGAGACGGGGT
TTCACCATATTGGCCAGGCTGGTCTCGAACTCCTGACCTTAGGTGATCCACCTGCCTCGGCCTCCCCAAGTGCTAGTATTACAGGTGTAAGCCATTGT
GCCCGGCTGAGAACTGTAATCTTTTCATGTTGATTTATTCTATCTAAGAACAAGGGATGTCTTTCCATTATTTCATGTCTACTTTTATGTCTTATAGG
GACATTTAAATTTTTTCCTTGTATGGATTTTGCACATTTCTTGTTAAATTTATTCCTGTGTCTTTAGGCCTCTTTGTTGCTGTTACAAATGGGGATTT
CTCCACCGTGATGTTCTGTTGATTTTTTCTGTATAAAAGGTCTATTTTTGTTTGTTTGTTTTGAGACAGAGTTTCGCTCTTGTTGCCCAGACTGGAGT
GCAGTGGCATGATCTTGGCTTATGGCAACCTCCGCTTCCCGGGTTCAAGCGATTCTCATGCCTCAGCCTCCCGAGTAGCTGGGGTTACAGGCATGTGC
CACCACACCCAGCTAATTTTGTATTTTTAGTAGACGCGGGGTTTCTCCATTTTGGTCAGGGTAGTCTCGAACTCCCGACCTCCGGTGATCTGCCCGCC
TTGGCCTCCCAAAGTGCTGAGATTACAGGTATGAGCCACTGCACCTGGCCCTATTTTTTTTTTTTTTAAAGATGGAGTCTCCCTATGTTGCCCAGGTT
AGCCTTGAACTCCTAGGCTCAAGTGAGCCCCCTGCCTTAGCCTCCTGAGTAGCTGGAACCATATGTGTGCCGCTGTGCCCCAGGGCTATTGATTTTGT
ATGTTAATTTTATATCCTGCTAGTTTACTGAATTATTTTATTATTTGAGTTAGTTTTATCATTGATTTTGGGGGGTTATATCTGCAAATAGAGATAGT
TTTATTTTTTCTTTACCCATTCTTAACCCTCTAATTGAGGTGTAATCCCATTGGCTGATACCCTTACACCAGTTTTGAACAGTAGCAGAGATAGTAAA
TATTCTTGCCTTGTTCCTGGTCTTAGTGAAAATGCCGTTAGTGTTTTGGAATTAAGTAAAATACAGGTTTTACGACTAATATATATGTGTGAGTGGCT
GAGAGAGTTCTAGATATTTAGATAGAACATGGGAACAGAGATTAGAGAGAGATTTCTATGAAGGCTTGTCAAATTCTAGGGCAATAAGTATATCACAT
TCAAAAGGTTTCATTTTTCAATTTGGGAGTAACAATGAGATATGCCATTACAGCAAGTCATTTGAATTATATAAATTGTGTTTATGCATTATAATGGT
CACAAAGAAAATTTTATGCTTGAATTTGTCTTGTAGTGTGTGCTGTATTTATGGAGTTTAAAGATTAATCATCCCAAAACATTGTTTCTGCTTCGGGG
AAATCATGAATGCAGGCATCTTACAGACTATTTCACCTTCAAACAGGAATGTAAGTATAATCACTCCTCTAGAACCTTTTTAGTTACCCTTTAACCTG
TCCGAAGTAAATCAAACAGAAAGAAAAAAGAAACTAATAGAATTTAAATGCTTCCTGTCCTGTTGTTATTTTTTTCACCTGCAAAGAACTAAGATTCT
ATCTTCTAAAACATTTATTTTGAAAATAACTTTTGGGTTCCCCCTTGTTTATAAAATAAATAATTCGGGATATTTGGAAAGCACAGAGAAGTCTAAAG
AAGATAAAAGTGTCCATAATCAGGTTGCAGTGAGCCAAGATCACGCCACTCTACTCCAGCCTGGGCGACAAAGCAAGATTCCATCTCAAAAAATAAAA
TAAAATAAAAGTGTCCATAATCATGCCACTTAGAGGTAATCACTGTTATCACACTATTTGAAATATGGTCTTCCATTCTTTTTTATTATTATGAAATA
GTTCAGGTATACATATATCTGTATATATTTTACAAATAAAATTGGGCTCATATGTACTGTTTAATACATTTCTGTTTCACCTAATGTAGTAGCATTTT
TTCCATATCATTAAGTATTTTTGACAACATGGTTTGTAAGTGTTATTTTAGTTTTTTTAAAGTATTCCATCTTACGAACACACAATCATATATGTAGC
CAATTTAGTAACAATCATAGGATATTTAGGTTGTTTCTAACTGTGACTACTGCTGTAATAGTAAAAATAAGTTTAATATAAAAGTAGAATTATTGGCC
GGGTGCGGTGGCTCACGCCTGCAATCCCAGCACTTTGGGAGGCCAAGGCAGGTGGAACACAAGGTCAGGAGTTCAAGACCAGCCTGGCCAACATGATG
AAACCCCTGTCTCTACTCAAAATACAAAAATTAGCCGGGTGTGGTCGTGCGTGCCTGTAATCCCAGCTACTCAGGACGCTGAGGCAGGAGAATGGCTT
GAACCCGGGAGATGGAGGTTGTGGTGAGATCCTGCCACTGTACTCCAGCCTGGGCAACAGAGCGAGACTCCGACTCAAAAAAAAAAGAAATAGAATTA
TTTTGAAAGTCAAAACAGGCCGGGCGCGGTGGCTCACGCCTGTAATCCCAGCACTTTGGGAGGCCGAGGCGGGTGGATCATGAGGTCAGGAGATCGAG
ACCATCCTGGCTAACAAGGTGAAACCCCGTCTCTACTAAAAATACAAAAAATTAGCCGGGCGCGGTGGCGGGCGCCTGTAGTCCCAGCTACTCGGGAG
GCTGAGGCAGGAGAATGGCGTGAACCCGGGAAGCGGAGCTTGCAGTGAGCCGAGATTGCGCCACTGCAGTCCGCAGTCCGGCCTGGGCAACAGAGCAA
GACTCCGTCTCAAAAAAAAAAGAAAAAAAAAAAGAAAGTCAAAACATTACTAGCTGATTTTAAAAAACAGATAACATGGTGGGAGGCTGAGGCTAGAC
GATCCCTTCAGCCCAGGAGTTCTACACCAGCCTTGGCAATACAGCGAGACTCCATCTCAAAAAAACAAACTCGTAGATTTCCTTGACTAGCATCTAGT
ACTCATAATTGGCTGCTCACTAACAAACAGTATGATGCTGACTATGTTGGGGATGTTTGGGAGATCCTGCCCCAGGGCATTTGCATTTTTAGTTCTTC
TTTTCCCAGATCTCTATCTGCGTATAAGACATACATTCTATTTCATTCATGTCATTTATTCCGAGATTTTCTCCTCTAGTTGCTTTTCTAAATCATAA
TCTCATCATTTTACCCTCATACCCTGTTTCTTTTCATAGTGTTTATCAACACCTGAGATTTATGTGTATTTATTTGTTGACTGTCTTTCTCTGCCACC
AGAATGTAAGCTCCATGCTAGCAGGGGTGTGTTTTGTTCACTGTTGTTTTCCCCAACACCCACAACAGTATTAGTTGCTCATAGAAGTTGCTTAGTAA
ATATTGGTTGAGTTCATTTGAATGAATGAGCATCTGTGATTTGTATGTTTAGAACACACTCTTGAAAAAGAATTTCTGGGTTAGGGGGCAGGCACATT
TTAAAGACTTTTGGCATACTCTAAAGTAGTCTTCCAGAAAATATGTATATTGGGTTATACGCCAAAAAAATTTTTTATTTTTGTGATGAAAGTACTGA
AATAAAAGCAAAATTTTACCTGTTTTGACTTCTAAAATTTTAGCATATTCTCTATAAAATAAGGAAGCTAAACTAAATATCTTGGGTCCCATCTGGCT
CTAAAATCTACCCCTTTTAACGAATCCTGAATATATAAATATACTTTAGTGTTCATATGGTTGATATTTTGATCATGGATTTCATTTTGTATCCAGAT
TTTTCTGCAGATTATTATCTTAGAAAAATATATTATAAAATATTTAAACTTTTATTCCAAAAATAATTTTCAAAGTTAAAAAAACTTTTACATTTGTT
TTCAGAGACTCAAAGTTTATTAATCTGCCACAGATCCCTTTACTGATAGGTAGATTGCTATTTATAGTGTTTCATATGCTCATATGAATTCTTGCTAC
CAGTATCTTCTTACATTGGAGATAGTAGTTGTAATGAAAACATCCCTCCTAGTAGAGCCTCAAAGAAAAGGTCTTTGTTATTAAAAGAAATTTTAGAG
GATAATCACTTCTTAAAAAGTCCCTCTGTCAAAAGTCAGCTGCAAGAGGGCAAGATACAAATCCTTCCAGCCCTTGGTGACTTACTGGAATAAACACT
TTAAATCAGCAGCTAATTACCAGGAAGTTTTAAATCTACTGTTGGTGGATGTTTAAATTGGTGTAAGTTGAGCATGGTGGCTCATGCCTGTAATCCCA
GTACTTTTGGAGGCTCAGGTGAGTGGATCACTTGAGCCCAAGAGTTCAAGACCAGCCTGGTCAACATGTTGAAATCCCATCTCTACAAAATAAAACAC
AAAAAATTAGCTGTGCATAGAGGCACTTACCTATAGTCCCAGCTACTCAGGAGGCTGATTGAGGTGGGAGGATCACCTGAGCTCAGGAGTTTGTGGCT
ATAGTGAGTTGTGATCACCACTACACTCCAGCCTGGGAGACATAGTGAGACCCTGTCTCAACAACAACAACAACAACAACAAAGATGTGAGGTATTTG
AAGAGCAATTTTGTAATATATTTATAAATTTTAAGTGCATGTCCCCTTGATCTAGCAATTTCATTTGTAGGAATTTATTATGCAGATTGTGTTACTTT
TTAAATGCTGCATAAAAAATTATCACAAATTTAGTGGCTAAAAAGAACACATTTATTATCTCATAGTTATTGTAGGTCAGAAGTCCAGGCCACAGTGT
GGCTAGATTCTCTGTATAGGATCTCACTGAAATCAAGGTGTTGCCAGGGTTTCAGTTCTCATCTGGGGCTTAGGGTCTTTATCCAAGGTCACTTGTTG
GCAGTCCTTGTAACTGTAGGATTAAGGTCCCTGTTGTCCTGTTAGTTGTTGACCAGGGATGACTCTTAGCTTCCAGATACTGCTTGCAGTTCGGTAAC
ATGTCGCCACCAGTGGTAGTTCCCAATATGAATGTTTGCTTTTTTCCAGGCCAGCCTGAATGCATCTCTCTGACCTTCTCTTCTGTGATCAGCTAGCT
GGAAAAAAACCCTCACTCAGTTTTTAAAAGGCTCACCTTAGGTCAGGCATGGTGGCTCATGCCTGTAATTTCAGCACTTTGGGATGCCAAAGGGGAGA
GGATCACTTGAGGCCAGGAGTTCAAGACCAGCCTGGGCAACATAGTGAGACCTCCTCTCTGCCAAAAAGAAAATTAAAAAACTAGAATTCAGTCAGCC
TGCATGGTGGTACAAGCCTGTAGTCCCGGCCAGTTAAGAAGCTGAGACTGAAGGATAGATTGAGCCTGGGTGTTTGAGATTACAGTGGGCTATGATTG
TGCCACTGCACTCTAGCCTGGGTGACAGAGTGAGACCCTGTCCCTAAAAATAAATAAATAAGTAAATAAATGTTCACCTGATTAGATCAGGCCCTCCC
AAGATAATCTTTCTTTTGCCATATAATGGGACAAAATAATGGGAGTCTCATCATACTTACAGTTTTCATCCACTGAAAGAGGAGTGGATTATACATAC
AAAGATGTAGGTCAATGCAGGTCATTTTAGAATTCGTCTACCACACAGATATACTCACAAAAAGATAGTAAAATATGGATGCTTAAGGATGTTCACTG
CAGTGTTAATTGTAACAACAACAACAACAACAAAAAGGTAATCTAAATGTCAACAAGACAGTGATGAAATAAGTGATAATATGCTTTTGAACAATGGA
ATGCTAGGCAGCTATTGAAAGACCAAGGTGGCCGGGTGCAGTGGCTCATGCTCATAATCCTACACTTTGGGAGGCCGAGATGGGGGCATTGCTTGAGC
CCAGGAGTTCGAGACTAACATGGGCAACACAGTAGTAAGACTCTCTCTCTACAAAAAATTTAAAAATTAGCTGAGTGTGGTGGCATGTGCCTATAGTT
GCAGCTACTTGGGAGGCTGAGGCAGGAGGATTGCATGAGCCCAGGAATTCAAGGCTGCAGTCAGCTATGATCACCACACTCCAGCCTGGGTGACAGAG
CAAGACTCTGTCTCCAAAAACAAAAGACAGGCAGACCAAGGTAGACCCATAATATGTACTGATATGGAGAGATGCTAAGATATTTTATAAGGCATTTG
CAGAGCATGTAAATGGTCATCTCCTTTGTAAATTAAAAATATATAATGTACAGAAGAAATCATTAACAGTGGGAGGTTGGGTTAGGAGGACTTAGAGG
GTTTTTTTGTTTTGTTTTTGTTTTGTTTTTGTTTTTTTAGATGCAGTGTTGACATGTTGCCCAGGCTAATCTCAAACTCATGCAATCCTCCAGCCTTA
GCCTCCTAAATAGCTGGGACTATAAGTGCATGCCATCGCGCATGGCTTACTTTTAATTTTATATCCTCTCTAGTATTGTTTGAATTATTTTTTACCAC
CACTATGTACTACTTTTATAATTATTATTATTTTTCAAATTGGTAATTTAAAAATTACCCAGTTGGGGCCCAAGCATTTGAAAAGCTTTGTAAGCACC
CAAGGTAGTTCAGCTGTGTGCTGCTGGTTAAGAACGACGGCTCTGGAGTCTTTAGGAAGCTTCTACTATCTGTCGTTTGGGAACCACTTCACCAAATA
CAAAAATGGGAAATATTTTATTTTATATTATTTTTCTAAACCAAATTTTAGGGTATTCCTTAACACCTTCATTTCTTACCTGGGAAAATTAAATAATG
TGTATAAGTTAACAAATGCCCTGGAATGTAATACTCGATAAAAGCTAATTTTATGAATTAGGAGTAGATACGTAGTGAAGAAAGCTGCCTATGGCTGC
CTTTGTTTTGAATTAAGTTTCAGAGGGTTTTTTAGGTGACATTTTTCTTAATTATTCTTTTGTCTTATCAGATTTTTTTTATTTTTAATTTTTATGGG
TATATAGTAGGTGTATCTATTTATGGAATACATGAGATATTTTGATACAGGGATGCAATGTGTAATAATAACATCAGGGATTTTTTTGACAGACTATT
TTTAGAAGATGGAACAATAGGTCCTGGAAACATTTTAAGTATCTCCTTTTGCCCAGAATTTCTTACCTTAGGTTTTTATTTGCTTTTCACATGATTTA
ACTGTAGTTAAACCTGATGGCTTTCTTTTTCTCCTATCTACCAAGATATTCCTTCTTTTTTATTATAATGCTGCATCTGAAATGTAAATAAATCTTGG
AACAAGCACACAGATTCAAGTATAAGTTTAAAATTTTATTCTTGTATAAGGAGAGAGCATGTGTGTTGCTCTGCATGGGTGTTTTTACTGATAAGCTG
ATCTCTCTTCAGTGGCCACTGTGGCTTTTAGAACTAGCATCTTATCATTAATAAGGGAGGCTTAGTTTCAGATTGTGCAGCTGTAATCTTGTTCATGT
TTTTAAGAGGGAGGTGGGTGAAGGCTTTTCTTGTATCAATCTTAAATTGCTTTATAATGCAGAGATTTTTTAAAAAATATTCCTTTCTTTATGGAAAA
GTAATAATGTTTTTTTTTAAATTGATGTATAATAGTTGTACGTATTTTTGAGGTACATGTGATATTTTTACTATATGTATACAATGTATAATTATTAT
CAAGTCAGGGTAACTGCAGTATCCATCGCCTCAAACATTTATTTTTTCTTTGTCTAACAGTTAAACAAAATTTATTTTTTTAAGAGACAGGGTCTCAT
TCTGTTGTCCAGGCTTGAGTACAATTGTGTAATCATAGCTCACTGCAACTTTGAACTCCTGGGCTTAAGTGATTCTCTTGCCTCAGCTTCCCGAGTAG
CTGGGACTGTAGGTGTGTGCCACCATGCCTGGCTAATTTTTAAATTTTTTGTAGAGACTGAGTCTCGCCTATGTTGCCAAGGCTGAACTTGAACTCCC
CAAAGGATCCTTCCGCCTTAGCCTCCCAGAGTGCTGAGATTACAGATGTGAGCTGCCATGCCTAGCCCCTAACAGTTAATTTTTGACATATCATTTGT
TTTTGCAAAAATATTTCTTCATTGTTATGTACTTAGAAAGCAAGAAACATAGTGCCTGCCTCCCTCCCTCCTTCCCTCCCTCCCTCCCTCCCTCCCTT
CTTTCCTTCCTTCCGTCTTGCTCTGTTCCCTAGGTTGGAACACACTGGTAAAATCATTGCTTACTACAGCCTCAACCTCCCAGGCTCAAGTGGTCCCT
ACACCTCAGCCTCCTGAGTAGCTGGGACTACAGGCACGAGCTCTCATGCCCAGCCTATTTTTATGTTTTTATGTATTTATTTATTTATTTTCTTTGAA
GGAACCTCGTGTTTATATTATTCCACAAATACTCTGGGTGCAACCTCATAGCTCTGGATCAGAGTTTCTAAAGTAAATGAATTTCTTATTTCCACGAA
CCTATTTTTTTTTTTTTTTTTTTTGATAGGGAGTCTCGCTTCGTCACCCAGGCTGGAGTGCAGTGGCACAATCTCGGCTCACTGCAACCTCCGCCTCC
CAAGTTCAAGTGATTCTCCTGCTTCAGCCTCCTGAGTAGCTGGGATTACAGGCACGCACCACCACGCCTGGCTAATTTTTATATTTTTAGTAGAGACA
GGGTTTCACTATGTTGGTCAGGCTGGTCTCAAACTCCTGACCTCGTGATCCACCTGCCTCGGCCTCCCAAAGTGCTGGGATTACAGGCGTGAGCCACC
ATGCCCGGCCCTTCCATGAACCATTTTTTTAAATGTATAGTTAAATAGCTTACTGACAAGTTCCAGAAACTCCTGTAATTTTACTAATGAAAGCAGTA
TTGTTATGACAACTGGATTTTTTAAAAAAATAAATACTGTTTGTCATTGCTCATTGCAGCATTTTTTAGAGTAGCAAAAGATTGATCTAATTAGATTG
TGGTATATCCATAAAGCGAAATACTAGGTAAATGTTAAAAAAATTACAAAGCTCTCCTGTAGGGGAGGCAAAATTTTACCTTTACTCTCAGGGTTTTT
CAGCTGGGCCTGAGAATTAAATTGACATGAGATAAATTAATAGGAGAAGAGCATATAAATTTATTTAATATAAGTTCTACATGACATAGGAGCCGCTA
TAAGGAAATGAAGATCCAGAGACTCAGAGTTGAACACTTACATGCTGAATCGGACAAAAAGTAGTAAGTTGTGAAAAAGTAACTAAATGATGTGAGGA
GGCTAGAAGATAAGAGTTATTTGAATTCAGTCTCAACTATTAATCCTTGGTGATAACAATGTTATTTTCCTTCTAGTACAGGGAGGGCATCTTTTCTT
TTTTTTTTTTAACTTTTATTTTAGGTTCAGGGGTACATGTGCAGGTTTGTTATATAGGCAAACTTGTGTCATAGGGGTTTGCTATACAGATTATTTTG
TCACCCAGGCACTAAGCCTAGTACCCAGTAGTTATTTTTTCTGATCCTCTCCCTCCTCCTACCCTCCATTTTTAAGTAAGTCTCAGTGTCTGTTGTTC
TGGGAGGGCATCTTTCAAATGGGAATTTCATCTCCTGCTTTTAAAAAGCAGCATGAGCCAGGCACTGTGGCACGTGCCTGTAATCACAGCTACTTGGG
AGGCTGAAGTGGGAAGATCGCTTGAGCCCAAGAGTTTGAGACCATCCTGGGCATGTAACAAGACCCCATTTCAAAAAAAAAGAAGAAACAGCATGAAG
GCCAGAGTGATCTTCTTGTACGTGTGGTTTTTCAAGTGCCTTTAACTCAAAGTTGTCAGTGTGCCAGAGTGGTGTATTTTGGGGTGGCATATTCTTAA
CTCCACTCTTCATCTGAAGATGTGGCATGATAGCCAAAATACGTTCTTAAGTACGAAAAGCAAGTAAAGAACAGTACATATATGCTATCATTTATGTA
AAAACCAAAAGGATGTTTTGGAAAGAGAAGAAACATATATGTCTGCATGTGTATGCATAGAACATCTTTGTAAAGACACCCAGGAAACTGGTAATATT
GGTTACCTGCAGAGAGAGGGCAGGAGGCACATTATACCCTTTTGTGGCTTCTAAAATTTGAGCTATGTTGGGGCCAGGCACAGTGGCTCACGCCTGTA
ATCCCAGCACTTTGGGAGGCCGAGGCGGGCAGATCACAAGGCCAGGAGATTGAGACCATCCTGGCGAACACAGTGAAACCCCGTCTCTACTAAAAATA
AAAAAAAATTAGCCGGGTGTGGTGGTGGGTGCCTGTAGTCCCAGCTACTCGGGAGGCTGAGGCTGGAGGATGGTGTGATCCCGGGAGGCGGAGCTTGC
AGTGAGCCGAGATTGTGCCACTGCACTCCAGTCTCTCAAATAAATAAATTAATTAATTAATTAATTAAATTAAATAAATAAAAAATAAAAAATTTGAG
CTATGTCTCACGCCTGTAATCCCAGCACATTGGGAGGCTGAGGTGGGAGGATCTCTTGAGGCCAGGAGTTCTAGGTTGCAGTAAGCTATGATAATGCC
ACTGCATTCCAGCCTGGGTGACAGAATGAGACCCTGTCTCAAACAATAATAATAAAAAATAAATAAAGCAAATAAAAATTGAAACAAATATATCATCA
GTGACTAGACCAGATTGTGATTTCTTGCAAGTAGTGCTGACTCTCAAAATTGCTTTCAGATAATTTTGTTGAAAATTTTGTTTTGTTAATGTTGTATT
ATTGATACTAGTTAGAACTTGTATCAGTGTCATCCTGTGGCTGTAATGCATTTTCCCTTCACTATAGAAATAACTGTTTTATGGGATATATCCTAAAA
TTTGTCTTGAAAATAAAGCAATTGAATCAGCTAATTTAACCTCTGGGAATTATGTATTTGGCTATTTAGATTCTCCTATTCAAAATCTTATTTAAAAA
TAATGATTAGGAGGAATATTAGAGGGTCATTAAGAGCATGAACAGGTTTAAGTCTCAGTTCTGCCAGTTACTTGCTTGCAGCTTTGGGCAGGTAAGCC
TAATTTCCTCAGTTTTTAAATGAAGACAGTAATAATACCTACTTCACAGGGTTTCTGTGAGTATTAAATACTTTCATCAGTTAACACAGTATGTACTA
AGTGCCAGCATTATTCTGCATATTTTACATAAATGAATTCATTTAATCCTCACAGCAACCCTGAATCTACTGTTTAGGGTGTTATTATTATTATGACC
ATAGATAAGGAAACCAAGGCACAGAGAGTTAAAATTTTTATCCAAGGTCCCATAGCTAGTATGTGGAGGGGGCTGCAGTTCTTCAAATCAGGCAACAA
GTTCCAGACTCCATTCTCTTCACCACCATGCCATGCCAAATGCTTAACTCACTGACAGACACATCATGAGCCTTCAATACATTTAAATAATAAAGGTA
ATATAGGCCAGGTACAAGTGCAGTACCTCATGCCTGTAATCCCAGCACTTTGGGAGGCTGGGGCAGGAGGATTGCTTGAGGCCAGGAATTGAAGACCA
ACCTGGGCAGCATATCGAGACCCTGTCTCTACAAGTAATTTTTAAAAATTAGCTGGGTGTGGTGGTGTGCGCTTGTAGTCCCAGCTACTTAGAAGATT
AGACAGGAGGACCACTTGATCCTAGGAGCTTGAGGTTACAGTGAGCTATGATTGCACTACTGCACTCCAGCCTAGGCAACAGTAAGATCCTGTCTCTA
AAAAAATTTTTTTAAAAGGTGGTATAATAATAAATGTTATTATATAAAGATTACATATTCATCATAAAAAAGAAAGAACAGCTAAACTAAGAAAAATA
TAGATACCATTGTCAGTCTAACCCCTCAGAAAATTAAATACTGGTAATTATATATGCAATTCATATACTTTGAAAAAATAAACATGGTGATATTTCAT
AGCCTATTTTATTCACATATTAATATAACATGAACTTCTTTTTGCTTAATTTTTGACCTCCTGTTTTAATTTACAAAATGACAATACACTTCCTTACT
TGTCCAGACTTGGCATTCTTTTTTGGAAGTAGGCTAAGGAATTATTTTGAGTCATTCTTGGGATTGAAAGGCATTTATCAATGTAAGATGTTATGGAG
CATAAATTAATAAGTAATTGAAATTCCTACCTCTATTCAAGTGTGGAACTCCCATGGAGGAATTCCTTACTGACACTGGCAACAGAGGGAACTCTGGT
TCCTAGGATTTTCAGGAGAAAAAGTCAATCACTGACCACAAATTAAAGAGTTTTCTGTTTGTTTTTTACTTCCAACAGACTTTATGGCCAATGCTGGA
TCTACTTTGAAATGGGCAAAATTTAAAACAAAGCAGAGTATAAGGATGTAACTGTAAGGAGAGGCAATGATGCATAAGACATGGTTCTTGTGAACTTT
GAGCTCCTAATAAAGTTAGCATATGTTGTGGAATAGATTATGCTGACTGCTACAACAGCAATACAAAATGTTCTGAAAACCTCAAAGAAACCCACAGG
CTCCCTTAATGAACTCAAACCCTAATCACCCAGTATTGGCGTTAAAGAGAAATCTTCACACTCAGGGATGGAAGGCCTTACAACCTCAAGCGTGGTCT
ACTGGTTTACACTTTCCTTAGGGATCTCTCTCTTAATCATTCCTGCCACCCATATAGTACTGCAGTCTGGCCCAAATCTGGACCCATCATTCTTCACT
TTATTCCCCAAAACCTTGCCAGGACCAGGCTCTGATCCTTAAATATGTCTTTCTAAAGGCTTAGAGATCACAAAAAGGAAATAAACACAGAAATAATT
ACCAACTAGATAGTCTGACTCACTCGATACAGCTTTGGTCTCTTGGTCCTGTAGGCCACCTCATCTTCCCAGAATCCTGGAAGAGGAGAAGGAGCTCA
CGGTTCTGCAGGGTTGCCTGACTATATTTCTCGTCTTCTCTACCCACACACATCCCTGAGCACAGCTTGACTTTTTGAAACCAAGGCCAAGGAAGAAG
AAAATGTTCCTTTTTCTCTTTAAACACCCATTTTCCAGTTTCCCACCTATTATTTCATGACTTTTCCTTTCCAGAGATTTGTAGAGACTCCTCCTTCC
ACCCACTGATGTATTTCCTTATTCTGTAGCCCCAAGTTGACAGCCACATCAAAATATAATTTATTAAGCCAAACATTTATTTTTTATTGTTTAGAGAC
AGAGGCTCACTCTGTTGCTCAGGCTGGAGTGCAGTGACACTATCGTAGTTCACTGCAGCCTCAAACTCCTGGGCTCAATCTATCCTCCCACCTCAGCC
TCCCAAGTAGCTTGGACAGCAGGTGTGTGCCACCACAGCCAGCTAATTTTGTTTTTCAATTTTCTGTAAAAACGAGGCCTTGGCCAGGCGCAGTGGCT
CACGCCTGTAATCCCAGCACTTTGGGAGGCCAAGGCAGGCGGATTGCGGGGTCAGGAGTTTGAGACCAGCCTGGCCAACATGGTGAAACCCCGTCCCT
ACTAAAAATACAAAAATTAGCCGGGCGTGGTGGCGTGTGCCTGTAGTCCCAGCTACTTGGGAGGCTGAGGCAGAAGAATTGCAAGAACCCGGAAGGCA
GAGCTGCAGTGAGCTAAGATCCCGCTACTGCACTCCAGCCTGGGGGACAGAGCAAGACTCCGTCTCAAAAAAAAAAAAAAAAAAAGAGGCCTCACTGT
GTTGCCCAGGCTGGTCTCAAACTCCTGGCCTCAAGCAATCCCCCGACCTCAGCCTTCCAAAACATTGGGATTACAGGCGTGAGCCACCACACCAGTTT
AAGCCAAATGTTTTTAGAGAAAATAGAATCAAGACATAATTTGTTTACAGACCTTAAAAATTATGAGCAAAAACATTATTGACATTTTATTGATGTCT
CTTAAGACTCCAGTTTCCACTTATATATCTTCAACACCCCTAAGAGATCTTTAAGACCCAATAAAATATTCAGACTCACAAGACTACCATGGACCCTA
GTTGTCATCCATATACTTAAAAGGATATAGGTCAGGAAGGGCAGCACTGGGCATTCCTTTTCAGTGGAGTGGTCCTCACAGGAGTGCATACCGCAGTC
CAGCAGCTTTCCATCTCTCTTGCCCCCTCCCATCTTCTCACCAGCTCAGGTACAAGGAAATATCCACAGTGGGAAGATTAGGGAGCCATCCTGGTATA
GAAGCCTCGTGCATCAAAAATGTCAATGTGTGTTGTAAAACTACCTTCAGAGGAGCCCAAAGCTATAGCTTCCCACCAGGTAACTGGGTAATTTTTAC
CACAAGCCATGTTGCCTAATAACACAGTGGACAGTCACCTTATCACATTTCCAAGCAACAGAGCTGGAAAGTTAATCCAAGGTCACACTTGTCTTTAA
GAGGAGAGAGGTATTGTTAACCTATTATCCACCACTTTTCAAAAGTATCTTTACAAAGATAATTGTGCTTTCAAATCATCTAAAATAAATGGCTCTAA
AGTAGCTTCATATTCTCCTGGCCTATATCCTTTTTCAAATGTGATACGTTTTAGTTCTTCCATTGACTGGTTTTCCTTTTTTGTTAGGTCGAATCAAA
TATTCGGAACAGGTGTATGATGCCTGTATGGAGACATTTGACTGTCTTCCTCTTGCTGCCCTCTTAAACCAGCAGTTTCTCTGTGTACATGGAGGAAT
GTCACCTGAAATTACTTCTTTAGATGACATTAGGAAAGTAAGTAATCTTTTATTATTCTCACAGGGAATATTTTTAAAATGTGTTGGGTTTTTTTGTT
GTTGTTGTTTGCTTTGAGACAGAGTCTCTCTCTTTCACCCGGGCTGAAGTACAGTGGTGCTATCTCAGCTCACTGCAACCTCCGCCTCCCTGGTTCAG
CCTCCCGAGTAGCTGGATACAGGTGCACACCACCACACCTGGCTAATTTTTGTATTTTTAGTAGAGACGGGGTTTCACCATGTTGGCCAGGCTGGTCT
TGAACTCCTGACCTCAACTGATCTGCCCACTTTGGCCTCCCAAAGTGCTGGGATTACAGGTGTGAGCCACCGCACGCCTGGCCTAAAATTTGTTTCTT
ATTGCAGTCAACCAATATATCTTTAGCTGAAATCTTTAAAATATAGATAATGAAAAGTAAGAAAATGGAAGTTACCTCTGATTTCATCACTATAACTA
CTGTTAACATTTTGATATGTACCTACCACAAATTATAATTTTTAATGAGCTCTATGTTTTTACCAACTAGAAAGCTAACATTCTTGTAGTATAGTCCT
GAAACTTTGAATTATATCGCAGTGCAAAAGGAAGGAAACCAGGGTTCTAATCACAGTCCTGCCATTAACTGTGTAACCTTAACCAAGTTATTGAACCT
CTTAGTCACATTTCTTCTCTATGAAATGAGGAAGTTGGAAGGGATGGTCTGAGGACACTGCCAGCTCCGAAATGCATTGGCTTTGACATTTTAGATGA
TAACGGTTTCATTGTTTTACTTGCCTTCCTTGGTGATTATTATTTCCAAGATTCTGTATCTTCATTATGGAAAAAGATGACCAATTTTTATTGTACAT
GGTTGTCTTATCTTCTCTACCTTTGAATTCTTCTACCTGTCTCTCTGTCTTTTTTAATCAAAGTGATAGATACTCATCCTCTCTTACAGGTACTGAGA
CTAAAAATTTTTCTGTTTATAAAACTATTTTCTCTGAAGAAAGTCTCCTCAGGGTAAGAGGGTGTACCTATATGTACTGGCTTTCCTCTTGCCTTACT
GTCTTTTAAGTAACCAGCATTTCTCTTTTACATAGTAAAATATGAATTTTATAATTCTGAATCAATTGTATCAATAAAAAGCTCTTTCACGCAGTAGA
AATTCTCTTCTTGGCATTCAGCTATTCTATTACTTGTTTTCTCTTGGTTTTTATGCTTTCAACTAAAGCAGCAGTGACTAAAGGCTTAAGTTAATAAC
TCATATTGAAATACCTCAACTGCATAGACTGGAAAGGATAAATGCCCTTACAAAATTAGCATTCTTTAAAATAAAGAAATCTCCACTGGAATGAGGTT
TGCAATGAACAGAACCATTGTAAAACATTTCTGCTGTCATGGTAACATTGGCTCACCTAGACAAATAAAATTAATTTTATATAGTCATAGTCTTTCTA
AAATAGTATATCTCTATAGTGGTTGATTTCACATTTGAATTAAATTAGCACTGCTTTACGATTAGGATGTAAGAGGGTCTTTTACTACAGTATGGTTA
GATGACAGGGATGTTTATAAAAATTTATTGCAGACCGGGCGCGGTGGTTCACGCCTGTAATCCCAGCACTTTGGGAGGCCGAGGAGGGCGGATCACCT
GAGGTCAGGAGTTCGAGACCAGCCTGACCAACATGGAGAAACCCCATCTCTACTAAAAATACAAAATTAGCCGGGTGTGGTGGCACATGCCTGTAATC
CCAGCTACTAGGGAGGGTGAGGCAGGAGAATCGCTTGAACCTGGGAGGCGGAGTTTGCGGTGAGCCGAGATCGTGCCATTGCACTCCAGCCTGGGCAA
TAAGAACGAAACTCCGTCTCAAAAAAAAAAAAAATTTATTTCAGAAAAATAATAAAAATTATGTATCTAAAATTGTTAAGTGTTCCTTTTCTTGCAGG
AAAGTCCTTTAGGGGCACACTGGCTTCTCTCCTAATACATTCTTGTGGGTGAAAGGGGTTTTTCTTTGACACATACTAGTTTCCAAGAAGCCTTTTGC
TCTCCTGATTTTTTTCTTTTGGCTTTTGTGTGTCTTCTAGTTAGACAGGTTTACGGAACCTCCCGCCTTTGGACCTGTGTGTGACCTGCTTTGGTCTG
ATCCCTCAGAGGATTATGGCAATGAGAAGACCTTGGAGCACTATACCCACAACACTGTCCGAGGGTGCTCTTATTTCTACAGGTAAGCTAGTCCTTGA
GGTCGAAAATTATGAAAGGAAACTGTAATTCATTTTATCAGATGATTTTTCAGCATTTTATATTTCAAATCTATGTAGTATAAGCACTCCTGTTTAAT
TTTTCGATTAATAGGAGGCAAGAAACTTGTTTGTTTGTTGGCTTTTTATATTTTCTTAGGTATATATCCTAAGAAGTGTAATACACCAAACATGATTG
GACATCCTGCAGTGCTCTGATATTTTTGTCTGACTCTAAGTGATCTAGACTATTTGTTCCTAAACATTATCTATACACTTTTTTAAAAAAAATTAAAG
CTTTCTTGGCTCTTTCCCCAGAGATTCTAATACACTAGGTCTGAGAGGAATCCTATTGTATTTTCAAAAGCTCTCCAAATTTTGTAACCACTAGTATA
GACTAAGATATAAAAAGAACAGGACATTGTGAACTAAGCCCAGTACATTTCCTGGAAGTTGGCTGTTGTTTTAAATTTCACAGGAGCTTTCATTATGA
TTAGACACTCAAAAATAGGAAGACAGGCAGGGCGTGGTGGCTCACGCCTATAATCCCAGCTCTTTGGGGTGCCTAGGCGGGCGGATCACTTAAGGCCA
GGAGTTCGAGACCAGCCTGGCCAACATGGCAAAACCCCATCTCTGCTAAAAATAAAAAAATTAGCCAGATGTGGTGGCAACACGCCTGTAATCCCAGC
TAATTGGGAGACTTCAGGCAAGAGAATCACTTGAACCTGGGAGGTGTAGGTTGCAGTGAGCCAAGATTGTGCCACTACACACCAACCTGTGCAACAGA
GCAAGACTCTGTCTCCAAAAAAAAAAAAAAAAAAAAAAAGGAAGACATAAGAGAAGTTGGAAATCTAAATTATTTTTAATCCCAGTATTTTATTGTGG
CAAATAATACATATGTTTATTTCCCTAAAAATCTCTGTTAATTGGCTAAGATAATGTGTTCATTAGAATACACACATGCACACACACATGAAACTTTG
ATTTCGCCATTACTTTTAGCATTCTTCTAAACAACCTTTTTTTCTTTAAAAAAATATTTTTTAAAATTTTTGTGGGTACATAGTAGGTATATATATTT
ATGGGTTATATGAGATATTTTGATATAGTCATACAATGTATAATAATCACATCAAGGTAAATGAGTTATCCATCACCTCAAGCATTTATCCTTTGTGT
TACAAACAATCCAATTATTACTCTTTTATTTATTTTATTTTTTGTTGTTTTTTTTTTCACCTTTTTTTTTTTTTTTCCTTTTTTGAGACGGAGTCTTG
CTCGGTCACCCAGGCTGGTGTGATCTCAGCTCACCGCATCCTCCACTTCCCAGGTTCAAGCAATTCTCCTGCCTCAGCCTCCTGAGTACTGGGATTAC
AGGCACCTACCACCATGCCTGGCTAATTTTTGTGTTTTTTAGAGATGGGGTTTCACCATGTTGGCCAGGCTAGTTTGGAACTCCTAACCTCAAGTGAT
CCACCCACCTCAGCCTCCCAAAGTGCTGGGATTACAGGCATGAGCCACTGTGGCCGGCCTCTTCTAGTTATTTTAAAATTTACAATTAAATTATTATT
CACTATAGTCACCCTGTTGTTTAAATCTCACAGTATCAAATACTAGGTCTTATTCATTCTTTCTGACTGTTTTTTTCGACCCATTAACTATCCCATTT
CCCCTCTTACTACCCTTCCCAGCCTCTGGTAACCATCATTCTACTCTCATCTCCATGAGTTCAGTTGTTTTAATTTTTAGCTCCTACAAATAAGTGAG
AACGTGTGAAGTTTGTCTTCCTGTGCCTGGCTTATTTCAGTTAACATAATGACCTCCAGTTCCATTCATGTTGTTGCAAATGACAGGATCTCATTCTT
TTCAATGGCTGAATAATATTCCACTGTGTATATGTACCACATTTTCTTTGTCCTTTCATCTGTTAATAGACACTTAGGTTGCTTCCAAATCTTGGCTA
TTGTGAACAGTGCTGCAATAAACATGAAAGTGCAGATGTCTCTTCAATATACTGATTTCCTTTCTTTTATATACATACATAGTAGTGAGATTGCTGAA
TTACATGGTAGTTCTGTTTTTAGTTTTTTTGAGGAACTCCAAACTGTTCTCCATAGTAGTGTACTAAACATTCCCACCAATAGGATATGAGGGTTCCC
TTTTCTCGACATCCTCACCAGCATTTGTCATTGCCTGTCTTTTGGATAAAAGTCATTTTAACTGGGTGAGATGATATCTCATTGAAGTTTTGATTTGC
ATTTCTCTGATGATCGGTGATATTGAATACCTTTTCATATACCTGTTTGCCATTTTATGTCTCCTTTTAAGAAATGTCTATTCAGATATTTTGCTCAT
TTTTAAATTGGATTATTGGATTTTTCCTATTGAATTGTTTGAGCTCCTTACATATTCTGGTTATTAATCTCTTGTCAGATGGATAGTAAAAAGTCTTT
TTTTCTTTCTTCTTCATTTATTTTATTTTATTTATATTTTATTTTATTTTGAGACAAGGTCTCTCTTTGTTACCCGGGCTGGAGTGCAGTGGCACCAT
AACAGCTCACTACAGCCTCAACCTTCTGGACTCAAGCAGTCCTCTTGCCTCAGCCTCCTGAGTAGCTGGAACTATGGGCATGTGCCACCATGCCCAGC
TGCTTTTTTTATTTTCTGTAGAAACAAGGTCTTGGCTGTTTTGCCCAGGCTGGTCTTGAAGTCCTAGACTCAAGTGACCCTCCCTACTTGGCCTCCCA
AAGTGCTGGGATTACAGGGATGAGCCCCACCCAGCCAACAACCATTTTTCATTCCTCCATGCCAACTTTTCCAGTGACTAATGTGATGAAATCATTTA
ACTTTTATTTCTTTTTACATTGCTTAAAATAAAGAGCATAATACATATTCATTGTTGTCAGAATAGAAATTGGTACAATTTCTGTGGACATCAACTTG
GCAATATCTTTCAAAGGTATTTGTACATGGCCTTTGATCCAGAAATTCTACTTCTAAAACTTAATCCTGTAGATATATAATTGATTCTCATTACTTGT
GGATTTCATATTTGTGAATTCCCCCACTTACTAAGATGTATTTGTAACACCCAAATCAGTACTTACAGAGGTTTCATAATCATTCATGGGAATGTAGT
GTGCAATGCAACAAAAAATTTGAGTCACCTCACTCACATGTTCCTAGCTGAGGTTGAACAAAATGATGCCCTACCTTCTTTTTGCAGCCCTCATATTG
TAAACAGGCATTCTTTTGAGTTTTATTTAGTGAAAGATGTTTTATGTTTTTGTGCTTTTCATGAGTGATTTCACTGTTTAGAATGGCCACCAAGTGTA
GTGCTGAAGTGCTGCTGTCTAGGGTCCCTAAGCACAAAAAGGCTGTGGTATGTCTTACAGAGAAAATATGCGAGTGTTAGATATAAGCTTTGTTTAGG
CATGAGTTATGGTGGTTTTGGCCATGAGTTCAATGTTAATGAATCAACAGTGTAAATGACCTAAAGTATTATTACACAGAAACACATCTGAAACAAGG
TTATATATTAATTGGTGGGTGAAAATGTTATGACCGGAGCCTCACAGGAACTTCACCCTGTATTTCCCTTAGCAGTAGTGTTTCATTATTTGCTAATT
CTGTTTTTGCAGCAACATTATAGAATGTAGCTACTGGGAATAATGAGATAAAAAATTTGACTCACCCAGCACACATGTTCCCAGCTGAGGCTGAACAA
AGTGATACTCTGCCTTCTTTTATCCCAGGGATAAATCCCATTTAGCCACAGTATATAATACTTTTAGTATGTTGTTGAGTTTGGCTTTCTAGTGTTTT
ATGGAGGATTTAAAAATCTATGTTCATCAGAGATACTCACCTGTACTTTTATTTATTATGGTGTCTTTGGCTTTGGTATCAGAGTGATGCTGGCCTCA
TAATGTGAGTTTGGAAGAATTCTCTCTTCTATTTTTTGGAAGGTTTAAGAAAGATTGGTATTAATTCTTTTTTTTAATGATTAATAGAATTCACCCAT
GAAACCATCTGGTCCTGTGCTTTTCTTTGTTGAGAGCTGTTTGATTACTGATTCCATCTCCATATTTGCTATTGGTCTGTTTGGGCTTTCTATTTCTT
CATGATTCAGTTTTGGCAGGTTGTATATTTCCAGACTCTATCCATTTCTTCTGGGTTTTCCAGTGTTTTGGTGTGTGATTATTCGTAATAGTCCGTTA
TGATCCCTTTTATTTCTGAGGCATCTATTGTAATGTCTCCTCTTTCATTTTTTGTTTTATTTATTTGAGTATCTCTTTTTTTCTTAGTCTTGCTAAGG
GTTTGTCAATTTTGTTTAGTTTTTCAAAAACCAACTCTTAGTTTTGTTGACTTTTTTCTGTTGTTTTTCTATTCTCTATTTGATTCATTTCTGCCCTA
AACTTTCCTTTCTTCTGCTTACTTTGGGCTTATTTGATTCTCCTTTTTCTAGTTTCTTGAAGTGTAAAATAAGGTTGTTTAATTCAGATCTTTTTTGT
TTTTTAAATCGGCATTTATTACTATAAACTTTCCTGTAGGTACTGCTTTTGCTGCATCCCATACATTTTGATATGTATATTTTTGTTTTCATTTATCT
TGAGGTTTTTTTTTTTTTAGATAGGATGTTTTGCTCTGTCACCCAGGCTCAATGCAGTGGCACAATGATACCTCACTGCAACCTTGAACTCCTGGGCT
CAAGCAGTCCTCCTGCCTCAGCCTCCTGAGTAGCTGGGACTACAGGATGTACCGCCATGCCCAGCTAATTGTTTTTAAAATTTTTAGAGGAGATGAGG
TCTTGCTGTGTTGCTTAGGCTGGTCTCGAACTCCTGACCTATCTAGAGATAATTTTAAAATTCCTTTTTGATTTCCTCTTTGACACAATAGTTGTTCA
AAAGTGTGGTGTGTAGTTTCTATGTATTTGTGAATTTTCCTGTTTTCTTAAGTTATTGATTTCTAGTTTCATTCCATTATGGTGGGAAAAGATACTTA
GGACGATTTACATCTTCTTAAGTTTGTTGATACTTCTTTGTGATTATATAGCCTAAAGAATATTCTGTGTACACTTGGGAAGAATTCTATTCTTCTGT
TATTTGGCAAAAAGTTTTGTATATGCGTATTAGGTCCATTGGGTTCATAGTGTTGTTCAGTTCTGTTGTTTGCTTATTGATTTCTGACTGGATGATTT
ATCCATTACAGAGAATGATGTATTGAAGTTACTTGCTATTATTATTATTATTTTGAGATGGAGTCTCGCTCTGTCGCCCAGGCTGGAGTATAGTGGCG
CGACCTCGGCTCACTGCAACCTCCGCTTCCTGGGTTCAAGCAATTCTCCTGCCTCAGCCTCCCAAGTAGCTGGGATTACAGGCATGCACCATCACGCC
CAGCTAATTTTTGTATTTTTAGTAGAGACGGAGTTTCACCATGTTGGCCAGGCTGGTTTCGAACTCCTGACCTCAGGTGATCCACCCACCTCAGCCTC
CCAAAGTGCTGGGATTACAGGTGTGAGCCACTGTGCCCGTCCTACTTGCTATTATTTTATTACTGTCAATGTTTGCCTTCAGATCTGTTGTTGCTTTA
TATATTTAGGTGCTTTGATGTTGGGCACATATATATTTATAATTGTTACATCTTTCTGTTGTATTGACCCTTTTATCATTATATAATGACTTTTGTTC
CCTCTTGTGATGATTTCTGACTTAAAGTTTATTTTGTCTGATAAAAGTTTAGCCACTCATGTTCTCTTCCGGTTACTTACTTGCATGGAATACCTTTC
TCTACCCTTTTACTTTCAGCTTATGTGTGTCTTTAAATCTAAAGTGAGTCTTTTGTAGACAGCATGTAGTTGGGTCTTGTTTTCATCCATTCACTCAT
ACTATATCTTTTGATTTGAGAGTTAAAGTAATGATTGATAGGTAAGGACTTACTATTACCATTTTGTTGGTTGCTTTCTGTTTTGTGGTTCTACTATT
CCTCTCTTTTTCTCTTTGTGATTTGATTTTTATTTTTGTAGTGGGGTATTTTGGTTCTTTTTATCTTTTGTGTATCTACTGTGCTTTTTTGTTCATCG
GGTTTGCATCAAACATTTTATAGTTATAGCACTCTGTCTTACGCTGAGAAGAAATTACTTTCAATGACGTATAGAAACTCTACACTTTAGTCCCCATT
CACACACCTAATGTAATTAATTTCAGGTTTTACTTTATTTTTGAGACAGGGTCTTGCTCTGTCACCTAGGCTGGAGTGCAGTGGTGCGATCTTGGCTC
ACTGCAGCCTCTGCCTCCCGGGTTCAAGTGATTCTCCTACCTCAGCCTCCTGAGTAGCTGGGACTACAGGCACACACCACCACACCTGGCTAATTTTT
TATATTTTTAGTAGAGATGGGGTTTCACCATGTTGGCCAGGCTGGTCTCTAACTCCTGGCCTCAAGTGATCTGCCTTCCTCAGCTTCCCAGAGTGCTG
GGATTACAGGCACGAGCCAGTGTGGCCAGCTGCATCTTTTAGTTTCTACTTGAACTTCCTTTAGTTGCTCTCATAAGGCTGGTGTAGTCATGACAAAC
TCCAGTTTTTGTTTGTCTGGAAAAGTCTTTATCTCTCCTTCATTCTTAAAGGGCAATTTTGATGAGTAAAGTATTCCTGGTTGGCAGGTTTTTTTTTC
TTTCTTTTAGTACTTTGAATATATTATCTCCCGCTTTCCTGGCCTGCAAGGTTTCAGCTGAGAAATTCACTGATAGTCTTATAGAAGCTTCCTTGTAT
ATTATATGACAAGTAGGTTTTCTCTTGCTGCTTTCAGATTTTTTCTTTTTGAGTTTTAACAACTTAATTATAATGAGTCTTGGTGTTTAACTTATTTG
GAGACCTTTGGGCTTCATGAATTGAAATGTCCCTTTTCCTCCCTAGATTTGTTAAACTGTTAGCCTTTAGTTTTTAAAAATAAGAAGCTTTCTGCCCC
TTTTTCTTCTCCTTAACTCCCATATGCCTGTATTATTTTCACTTGATGTTGTCCCATAAGTCCTGTATACTTTCCACATTCTTTTTTATTCTTTTTTC
TTTTTGTCCCTCTAATGCTATCATTTCAAATGACCTGTCTTTTAAGTTTCCCAGTTCTTTTCTTCTGCCTGAGTCTGCTGTTGAAGCTGTCTCTTGAG
CTTTTCAGTTGAGTCATTGTATTCTTCAGCTCCAGGATTTCTATTTGCTTCTTTTTTATGGTTTCTGTTTCTTTATTAAACATCTCTCTTTATTCATT
TATTTGCTTTCCTGATTTTGTTTAGTTGTAGTTCATTGAGCTTCTTTAAGATGATTATTTTGGATTCTTTGTCATGCAATTCATACATCTCTATTTAT
TTAGGGCCAGTTACTAGAGGTTTATTAGTTTCCTTTGGTGGTGTCATTTTTGCCTGGTTCTTTATAATTCATATAGCCTTGCAATGGAGTCTATGCAT
TTGAAGGAGCAGGGGCCTTCCAGTTTTTATGGACTCGTTCGCCAAGTAAATACCTTCTCCTATTGAGTCCCTGGGTTGATGGGATTGCCTGTGGTATT
GTGATTAAGCAGGGCTGAAGCTAGATTATGGGGCTACATTAGGGTTCACAGTCAGTGGGCGTACCACTAGGGGCATGGACAAGTATGTCCCATGGATG
GGGAGAGCTGTCTCTGGAACTCAATAGGGTGGGACCCAAGCTGGGTCCCAGGCTGGTTTGGGTTTATGTTTGGGTCCAATCAGCAGCCCTTTTACCAG
AGGAATGGACAGGCATGATTTCCACAAGGCCCCTGTTAAGGCAGGACTTCCTCTTGGCCACAGTAGAGCAGGGCTGGAGCCAGGTCAGAGTGCTGCTT
TGAGTCTGCAGTTGGGTCTGGTTGGTGGATCTGTTACTAGGGGCATCAACAGACATGGTTCCTTCCCAGGATGGATGAGGCTGGTGGTAGGACTGCAG
ACAAGTGGGACTGGAGCCGGGTTTACAAGGGACAGGCTGATTCTGGGTCTGTAGCTGAAAGGTCAGTGGGCCTGCCTTCTCCAAGCAGCCCTTCCTAG
TCTTGGGCTCCACCAAGGTTTCATAACCACCTACCTCAATCCTAAGGTTCTTACAATGGCACTTATGGCCATGGATGGCTTCCAAACTATTGTTTCTG
TAGGAGGACCAGGGCTGGGGACATTCTCTTTTGCTATCTTGCTTACGTCACCTACAACCTACTTTAAAACTAAATTTGTTCTAATGAGCATAATGAGA
AACAAAGATTTTAAAAATAAACTAAATAATTAATGCCTAAGTAATTCCAGGTTAATTTTGTATACTCCAGTTCTTCCTATCAGGTATTTGTCATTCAG
AGAGGTAATAGTATGAAAAGAATATTTTAGGAGTATATGAGAATATTGTATCTTTTTTTAGAATATGGTTGGCTAAAATATTTACATCTGCTCCTAAA
ATATTCCTGCATAAGCTTGCAGTGAGCCGAGATTGCACCACTGCACTCCAGCCTGGGCGACAGAGCAAGACTCCGTCTCAAAAAAAAAAACAAACCTA
TATATATACATATATATGTATATATGTATTCCTGCATAATATTATCTTGGTGTTTATATTACCTGTGAAGCTGTAAGTATATTCTTTTATTTCTGTAA
CAATTTGACAATTGAAGAAAAGTATGTGTTGAATTTTTAGCATCTACTACACACACACGCACACACACACACACACACACACTACTGTTGTACTTTGT
AATATCAAGACACTGATAAAAGTTTTGGTGTTGTATTGTTATGGATTAAGACAAAAAAATTAAAAGATGAGATGGAGGAGCTCTTTCTATTGTAGGAT
TATCTATTGTTTTCATGTTTGATCTGTAGTGAAGTATATTAGTGAAATAGAGAAACAGAATGATGAGTACTAAGCTATTCATAAAATTTCAGTAGTGT
GTAATACTTGTAGTTTTGAAACAAAATCAGCTAATATCACCTTGACTTTTCCCCATCTTCCTATTAAAAAAAATTGTTTTAATTCTGGATTTATGTTT
TCTAATTTTCTTTTCCAGTTACCCTGCAGTTTGTGAATTTTTGCAGAACAATAATTTACTATCAATTATCAGAGCCCATGAAGCCCAAGATGCTGGGT
AAGTTACATTAAAATTGTACCAAATATCGCTGCAGAGTCTTTGCATTTAATATGCAGACAGATGGACTTTCATCTCTTTTTCAGGTATCGAATGTACA
GGAAGAGCCAAGCCACAGGCTTTCCATCACTTATTACAATTTTCTCTGCCCCCAATTACCTAGATGTCTATAACAATAAAGGTAAAGGAATCCAGCAA
TATTTGAGTTTGAATTTATGAGTAAACGTGAGCTCTGGTTTAATTGTATGTACGTATGTGTGTTTGTGTCATTTTAAAACATAAATTTTAAATAAGAA
AAAAAGGGGTCTTAGTTGAAACAAAGGCCAAGAAATTTAGAATTTATGCTGCTGCTTGGTCTTTATCTCCTAAGGATTACTGGCTTCTTTGTGAGTAT
AAATAATGATCATAATAGGTCAGATTGATACTACAGAATCAAAGTATAAGAAGAATAAATTGCTTCAGGCTCATGCCAGAATAGTTAGCATAAATTAT
TGGTTTTGGTGAATTTAATGTGTTTATAACAGTATCTCAAAGTCACCATTGGTATTTTAATCTTTGTGAATTATTAAACTATTTTTGTTTTTCTTGAG
ATATGGTCTAGGTAGAGTGACCTAGTGTACAAAGTCAAAAAAAAACCTTCATTTTTAGTTTAAACCCTGCCAGAAACGAATACTGTGACTTTGAGTGA
GTCACATTATCTTTCTAATCTTCAGTTTCCCATATGAAAAAAGAAGAATGGATATATACATGCCTTTTGTTCCAGAAATTTTACTTCTAAGACTTTAT
CCTGTTAAAGTCTTTTTTTTAATTTAAAACTTTTATGATTAATAGTTTATAAGTTTTCTATCGAGGCAGTGGTCCTACTAAAAACTTCATATTTCTGT
AACTATTTTGTTGTTTATAAAGTATGACTATAAATTAATAGGACTTTGAAAATAAACATGCCAGAACAAACATATCCAAATGAGTATTGTCCCTTCAA
AGTAGTTACCTTGGGAGGCTACACACCTATTCCAACAATGCTGTCCTTGTTCAAAAACATTTTTGGAACTCCTCTTTTGGCATTGCCTTCAGAGTCTG
CGACACATTCTTTTGATTATCCTCAATGATGGGAAATCTTCGTCCTTTGAGGGTGGATTTGATTTTTGGACAGCCAAAAGTCATTCGGAGTCAAGACT
GATAAATAAGGTGAGTGATCAAGCTAGATGATACCATTTGGGGTCAGAAACAAGGTATGATTATAAGGTAATGAGACTGGTTTTTCTCGTGTTGCTGA
TACAGTGGCTCTGGAGGCACTTCCAAAAGAGGAGTTCCAAAAACGTTTTGAGTAATGGCAGCATCTCTGGATTAATTATATGGTAAGGCAACAAGTTG
TGCTGAAAAGAACACAAAGCAAGGAGTCAGTCTAGTCCTGCTTCACTTTTAAGTGTGTCACATTTAACAAGATACTTCTCTGAGCCTCATCTTTAAAA
TATCTGTCATATGGGGTTGTTGAAGATAAGTTGGAATAAGGAAAATGGATATTAAAGTGTTGTTCAAAATATAAATTACTATAATAATACAGATTTTA
CTGTTATTGTACCTGATGTGATAATATCAAAGGACCACACTCATCTGATAAAAAGGTCTGGTGTGTTTGTAGCTGGTGGTTTCTTAAGTCTCATTACT
TTATAGTTATACCTTGTATATGCAGTTGGGGAGATAAGTTTCAAAGATGGCCAAAATATAATTAATATTTACTCTATTTAAAAGTAAACCATGGAATG
GCACATCTCATTTAAATCCGATTTGGGTCAGTCATTTTAGTGTGTCCTCCAGTGACTTGTCTATTTTAAAAAGCATGTCTTTAACTTAAGTTCTGTCC
TAGTCTTATGTTTTCATTTAACTTAGCATTCTTCTCTGAGCCTTATCTGTCCTACAATGCTCTGCTTCTCCCTAAGTTCATTTGTTATTTCCTTTTTC
CTGATATTTCCAGTATATTAGCAACTTAGGTTTTATTCTTATTTATTCTCTTATCAATGGTGTGGACTGTTTGTGTTGTTAAACGTCTTCAAGGACTC
TTGTGGCCCTAGATCTCCTATTTGTAATGAAGGTGTTTCTTTCTCACTTTCTTTTTTAACTTCATCAAAAAGAGTATCTATTGATTGCTCAATGGATT
TATCAGTGTGCCAAGCTGAAACACGCCACCTAAGTCAGCAGGCACTATACAGCTCACTCACTGCTAGCTTGTTTTTTGTCTCAATCTAACCTGGTTTT
GTTCTTGATTATTTCTTTAGCAAACTTTTAACATCACAGTCCTGGCCAGGCACAGTGGCTCATGCCTGTAATCTCAGCACTTTGGGAGGCCAAGGGGA
GGATCACTTGAGGCCTAGAATTCAAGACCAGCCTGGGCAGCAAAATGTGACCCCATCTTTACAAAGAAAAATTTAAAAGTTAGCCAGGCAAGGTGGAG
CATGCCTATAGTCCTAGCTACTCAGGAGGCTGAGGCAGTAGGATCCTTTGAGTCCAGTAGTTCGAGGCTGCAGTGAGCTGTGATCACACCACTGCATT
CCAGCCAGGCAATAGAGCAAGACCCTGTCTAAAAAATAAAAAAAAAAGTAAAAGCCACAGTTCATTCTACCTGAGTATTCTGTCGGCTTTGACATAAT
TAGTAACTTTTATCTCAAAACAGTCTTATTTTCATTGGTTGTTTCTTAAGCATTTTAGAAGGAAAAAGGCAGAAAATGTGGCATTTGAAACAGTCCCT
GGGGGTACTCAGTTACTCCCCATACAAGTGCCTTCTTTTCTGAAAAAGGGAACTCTATTATTCTTTTTCTTCTTCCTTTTCTTCTACTGTTGCTCTTC
TTTCTCTTTCTCTCTTTTCTTTTCTCTCTCTTTCTCTCTCTCTTTCTTTTCTTTCTTTTTCTTTCTGCTTCCTTTTCTTCTTTCTTTCTTTCTTTCTT
TCTTTCTTTCTTTCTTTCTTTCTCTCTCTCTCTCTTTCTTTCTCTCTCTCTCTCTTTCTCTCGGTCTCTGTCACTTAGGTTGGAGCACAGTGGCAGGA
ACTTGATTCACTGCAGCCTCGACTTCCTGGGCTCAAGTGATCCTCCCACCTCAGCCTCCCAACTAGCTGGGACCACAGGTGCATGCCGCTACACCTGG
CTAATTTTAAAATATTTTTTATAGAGATAGGGTCTCGTTATGTTGCCCAGACTGGTCTCAAACTCCTGAGCTTAAGCAGTCCTCCTGCCTCGGCCTCC
CAAAGTGCTGGTATTACAAGTGTGTGCCACCACACCTGGCCTCTTTCATATTATTTTTTTTTTTTCAGAGACAGAGTCTCACTGTGTTGCCCAGGCTG
GAGTGCAGTGGTGCGGTCTTGGCTCACTACAACCTCTGCCTCCCGGGTTCAAGCAATTCTCCCGCCTCAGCCTCCCGAGTAGCTGGGACTACAGGTGC
ACGTCGCCACGCCTGGCTAATTTTTTTTATTTTAGTAGAGACGGGGTTTTACCATGTTGCCCAGGCTGGTCTCAATCTCCTGAGCTCAGGCAATCTGC
CCACCTCGGCCTCCCAAAGTGCTAGGAGTGCTCTTTCATATTCTTTAGACTTCTTGGTATCTACAGGTATTTCCTAGTTCCACTAGTTACACGAAAGA
CCTTTAATCATGATTTTTAAAGATTCTTATTCTTGTTTTCTTCTGTCCACTGGGAACACACCCAAAAGCAGTTTTATAAGCTCTTGTTTCCAGTCATT
CACTGTTGAGACTCACCTCCCACTTCTTTTTCCTGGGTAGAAATTGAGGGAGTTGTTGAATAATGATATATGACATATTTACCTAAACTAAATAAAAT
ACATACACGTGAAAATAATGGAACATGTACATCATAGCCAGTGTTAGGCTTTTTAAGTGCTAGAAACTATATTTTAATTTATTTTCTTAATAATATAT
GGTGTACATTTTAGACATTCAATAACTTAAATTTTAAGAACACTAGATTATCCTGTAGCAAAGAAACAGGAATGAGGACGTGACCTATATCTGTCATT
TCTGGAGAACTGTTACACCCTGTTTTATTTTATGACCACAGAATAATTAAGAGAATAGTGCAAACAACATTGCTGGTGATAAAGGCTAAGGAATTGGA
TTTTAAGACTTTCAAAATATAAAGAAAATCAGTTTGGCATCAAAGTATGTGTGCGACTATCTGAATGGTTAATATTAAACATAGCATGCTGGCAATAC
ACTGCTGTGTATTGGAGAAATCGGCAATTTTAAAATCCGCTATATTGAATACAGAGATGACTTAATCTAATTTTGAACATTTAATTGTAAATTGCTTC
AAGTTTGCCATTTGTATCTTCCAGTATGAGTTGATAACTGCAATATATAAACTCCCAGAGAGTGTATACAATCTTTGACTCTGGCTAATGACTAGCAA
AACAGGCTGTGTCCATTTAATGAGTAATTTTTGCCTTCAAAGGAGAGTTGTAATTATAACCATTTGGCCTATAAAAGGAAATTATGGGGAAATTTTCC
TCACGTGGATCTTGAGGTTTATATAGATAGTTACTGTGCAGTGACTCCAAAGTTTCAGTTTCTTTTGTACTTTTATTATATGAGCTCTTCAGGTAACT
TTGTTCCTTGGAATAATTCTTAAGAGAATGAACTGAATAAAAGGCTTTAAAATTTTCCATCTGCCCTTTACCTAGTCAAGTCCTGACTTTACAAATGG
AAAGTTCTCGAATTCTGTGTAGCAGGTACACAGCTGTACAAAGCATACCACTTGCCATGCCATGTTCCTCTGTGCCAGATTTTCTTGCTTTTTTCCTT
TTTAGCTGCTGTGTTGAAATATGAAAACAATGTCATGAATATCAGGCAGTTTAACTGTTCTCCACACCCCTACTGGCTTCCAAACTTTATGGATGTTT
TCACATGGTCTTTGCCTTTTGTTGGGGAAAAAGGTAAGAGAACTAAAGCACATGTCTCATCAGTTGTTTGGTGACTGAGCATACCTTATATTTGCATG
AGCCCCTCCATGATTCAGAAATGTTTTCTAATTTTGTTTGTTCTCTACATAGATTTTTTTTTTTTTTTTCTGAGATGGAGTCTCGCTCTGTCACCCAG
GCTGGAGTGCAGTGGCACGATCTCAGCTAACTACAACCTCCGCCTCCCAGGTTCAAGCAATTCTC
HUMAN SEQUENCE - mRNA
GGGCCACCCTTAGCAGCGGTCGCGGTCGGTGCCGAAGCGGTGTTCCCCGCCTTAGCCGCTGCGCCTCCCAAGAGAGCGGCCGGTGGGCCCTCGTCCTG
TCAGTGGCGTCGGAGGCCGGCCTGCGGTGGCCGCGCCCTTCTGGTGCTCGGACACCGCTGAGGAGCCGGGGCCGGGCACGGCTGGCTGACGGCTCCGG
GCAGCTAAGGCTGCCCGAGGAGAAGGCGGCGGCCGCGGCGTAGGCGCACGTCCGGCGGGCTCCTGGAGCCTGGAGGAGGCCGAGGGGACCATGTCCGG
GAGGCGCTTCCACCTCTCCACCACCGACCGCGTCATCAAAGCTGTCCCCTTTCCTCCAACCCAACGGCTTACTTTCAAGGAAGTATTTGAGAATGGGA
AACCTAAAGTTGATGTTTTAAAAAACCATTTGGTAAAGGAAGGACGACTGGAAGAGGAAGTAGCCTTAAAGATAATCAATGATGGGGCTGCCATCCTG
AGGCAAGAGAAGACTATGATAGAAGTAGATGCTCCAATCACAGTATGTGGTGATATTCATGGACAATTCTTTGACCTAATGAAGTTATTTGAAGTTGG
AGGATCACCTAGTAACACACGCTACCTCTTTCTGGGTGACTATGTGGACAGAGGCTATTTCAGTATAGAGTGTGTGCTGTATTTATGGAGTTTAAAGA
TTAATCATCCCAAAACATTGTTTCTGCTTCGGGGAAATCATGAATGCAGGCATCTTACAGACTATTTCACCTTCAAACAGGAATGTCGAATCAAATAT
TCGGAACAGGTGTATGATGCCTGTATGGAGACATTTGACTGTCTTCCTCTTGCTGCCCTCTTAAACCAGCAGTTTCTCTGTGTACATGGAGGAATGTC
ACCTGAAATTACTTCTTTAGATGACATTAGGAAATTAGACAGGTTTACGGAACCTCCCGCCTTTGGACCTGTGTGTGACCTGCTTTGGTCTGATCCCT
CAGAGGATTATGGCAATGAGAAGACCTTGGAGCACTATACCCACAACACTGTCCGAGGGTGCTCTTATTTCTACAGTTACCCTGCAGTTTGTGAATTT
TTGCAGAACAATAATTTACTATCAATTATCAGAGCCCATGAAGCCCAAGATGCTGGGTATCGAATGTACAGGAAGAGCCAAGCCACAGGCTTTCCATC
ACTTATTACAATTTTCTCTGCCCCCAATTACCTAGATGTCTATAACAATAAAGCTGCTGTGTTGAAATATGAAAACAATGTCATGAATATCAGGCAGT
TTAACTGTTCTCCACACCCCTACTGGCTTCCAAACTTTATGGATGTTTTCACATGGTCTTTGCCTTTTGTTGGGGAAAAAGTCACAGAGATGCTGGTA
AATGTGCTCAACATATGCTCTGATGACGAACTGATTTCTGATGATGAAGCAGAAGGAAGCACTACAGTTCGTAAGGAGATCATCAGGAATAAGATCAG
AGCCATTGGGAAGATGGCACGGGTCTTTTCAATTCTTCGGCAAGAAAGTGAGAGTGTGCTGACTCTCAAGGGCCTGACTCCCACAGGCACACTCCCTC
TGGGCGTCCTCTCAGGAGGCAAGCAGACTATCGAGACAGCCATCAGAGGGTTCTCGCTTCAGCACAAGATCCGGAGTTTTGAAGAAGCGCGAGGTCTG
GACCGAATTAATGAGCGAATGCCACCCCGAAAGGATAGCATATACCCTGGTGGGCCAATGAAATCTGTAACCTCAGCACACTCACATGCTGCGCACAG
GAGCGACCAAGGGAAGAAAGCCCATTCATGACTTAGAGTCCTGCCGTGCTCAGGTGGATCTAAAACTCAAGAACAAATTCTATTTATTTATTATTGGA
AAATGAAAAGCAACTCAAAACAACTTCAACCTGGAGGTGCATTTATAATTCAGTCTGCATTTATTCTGTAAAAAGGTGACTGTTTTATAAATTCTTTT
AATTTATGTTCAATATATATAAAAAGTGCATCTGTTTTGTTTTTCCCTTTTTTCTCCATAATTTTAAGAAATGAATCTGATTGTTGTCAACACATTTG
TGAAGTCTTGTGCTATAAAGGGGAACTTCCCCTAATAAAAGGGCCTTGGAAACCTCAAACCTGGGTTTCTGACCCC
HUMAN SEQUENCE - CODING
ATGTCCGGGAGGCGCTTCCACCTCTCCACCACCGACCGCGTCATCAAAGCTGTCCCCTTTCCTCCAACCCAACGGCTTACTTTCAAGGAAGTATTTGA
GAATGGGAAACCTAAAGTTGATGTTTTAAAAAACCATTTGGTAAAGGAAGGACGACTGGAAGAGGAAGTAGCCTTAAAGATAATCAATGATGGGGCTG
CCATCCTGAGGCAAGAGAAGACTATGATAGAAGTAGATGCTCCAATCACAGTATGTGGTGATATTCATGGACAATTCTTTGACCTAATGAAGTTATTT
GAAGTTGGAGGATCACCTAGTAACACACGCTACCTCTTTCTGGGTGACTATGTGGACAGAGGCTATTTCAGTATAGAGTGTGTGCTGTATTTATGGAG
TTTAAAGATTAATCATCCCAAAACATTGTTTCTGCTTCGGGGAAATCATGAATGCAGGCATCTTACAGACTATTTCACCTTCAAACAGGAATGTCGAA
TCAAATATTCGGAACAGGTGTATGATGCCTGTATGGAGACATTTGACTGTCTTCCTCTTGCTGCCCTCTTAAACCAGCAGTTTCTCTGTGTACATGGA
GGAATGTCACCTGAAATTACTTCTTTAGATGACATTAGGAAATTAGACAGGTTTACGGAACCTCCCGCCTTTGGACCTGTGTGTGACCTGCTTTGGTC
TGATCCCTCAGAGGATTATGGCAATGAGAAGACCTTGGAGCACTATACCCACAACACTGTCCGAGGGTGCTCTTATTTCTACAGTTACCCTGCAGTTT
GTGAATTTTTGCAGAACAATAATTTACTATCAATTATCAGAGCCCATGAAGCCCAAGATGCTGGGTATCGAATGTACAGGAAGAGCCAAGCCACAGGC
TTTCCATCACTTATTACAATTTTCTCTGCCCCCAATTACCTAGATGTCTATAACAATAAAGCTGCTGTGTTGAAATATGAAAACAATGTCATGAATAT
CAGGCAGTTTAACTGTTCTCCACACCCCTACTGGCTTCCAAACTTTATGGATGTTTTCACATGGTCTTTGCCTTTTGTTGGGGAAAAAGTCACAGAGA
TGCTGGTAAATGTGCTCAACATATGCTCTGATGACGAACTGATTTCTGATGATGAAGCAGAAGGAAGCACTACAGTTCGTAAGGAGATCATCAGGAAT
AAGATCAGAGCCATTGGGAAGATGGCACGGGTCTTTTCAATTCTTCGGCAAGAAAGTGAGAGTGTGCTGACTCTCAAGGGCCTGACTCCCACAGGCAC
ACTCCCTCTGGGCGTCCTCTCAGGAGGCAAGCAGACTATCGAGACAGCCATCAGAGGGTTCTCGCTTCAGCACAAGATCCGGAGTTTTGAAGAAGCGC
GAGGTCTGGACCGAATTAATGAGCGAATGCCACCCCGAAAGGATAGCATATACCCTGGTGGGCCAATGAAATCTGTAACCTCAGCACACTCACATGCT
GCGCACAGGAGCGACCAAGGGAAGAAAGCCCATTCATGA

TABLE 7

MOUSE NOMENCLATURE
ICSGNM N/A
Celera mCG9110
HUMAN NOMENCLATURE
HGNC N/A
Celera hCG1641650
MOUSE SEQUENCE - GENOMIC
TGATAGGAGAAAGGGTCTGTTGGTTACCATGTGGCATGGCCATTGTATCTGTAAGAGAAGGATACTGTAGCCCCTCCCTAGAACAGCCATCCCT
CCAGTATGCCAGTCTTCTCCTGGTTCACTGCACAATAGCTTTCTGACTTTACCCTTTGCCATCTTCCTGTCTCTTTATTTTGTTTGAAAAATAC
TTATTTTTATGTATTCTTTGAAAGTTCCATACATGTATACATTATGTTTGAGCATATCCACCCAACATCACATCTCTCCAATGCCCCTACAACA
CCCCACTCTGTCTGCACCCAACTTTGTTGTTGTTATTAGTCCTGCATTCAGGTCAATTAGTGTTGCCCATATGGACGTGGTATGTGACATCATC
ATACATTTTGGTGTGGGGACTCTACCATCAGCATGTGGCTAGAGATTGTCCCTCTTTCGGCAGCCATCAATTGCCAGTAGCTTACTCACCAAGG
TGGCATTTTGACAGGCTTGTTCTCATCTAGATCTTGTACAGGCAACCACGGCTACTGTGAGATGACACATTGCCTCATTTTTAAAGATAGAATC
GTACACTATAGCCAAGGCTAGCCTAAAACTTACTGTGTGTGTATGTGTGTGTGTGTGTATACACTCATATAGCTAGACTTGAACTATCAACAAT
TGGATCTTACACTATAGATGAGGCTGGCCTAGAACGTACTACATATTGCAGGCTGAGCTTGAACTATCAACTATCCTCTAGTCTCAGCCTCCCA
AGTGCTGGCATTACACACATGAGCCGTCATGACTAGCTTCCCTTCGTTCTTAGCACATCCCATGAGGGAGCATCTTAAAGGGCCCTTGACCCTT
GCACACCTCATTAGATACCTCATTTGTTTCCCTGAGTATAATGAGGTCTTAAGTTGAACCTGGACATGTGGTCCAAAACGCTTATTAAATCAGA
TTACTTTCTCACACTATTAGACAAGGAAATCTGTGCAGACAAATTTTGTGTCTATGATATGAAGAATGTCCACCTGCAGGAGACATTAGCAGTG
TGACTATTGATTGTGAAGTAGTTCGCCTAAGAACAGAGGGCAGCAGACACAGGAAGCAGCAAGGAAGGGAACAAGAGCAGACAAAGAATGAAAG
CAAAGATGCCCTTTCTCCAGTCCTCCCGCCCTCGAGCCCCTTGTTTGAGCAGCTTTTGAGAAGGGTTGTTTTTTGTTGTTTCTTCTTCTCTTTG
TTTTTGTTTTGTTTTGTTTTGTTTTCATAGACTCTTGCCTGCTGAGGACCAGGCTAATCATACACTACTATGCCAGGAGGTTTTTTGTTTTGTT
GTTGTTTTGGGTTGGATTTTGTTTGTTTGTTTGGTTGGTTGGTTATTTTTGTTTGCTTGTTTTGGTTTCTTTGTTTTTGTTTGTTTGTTTTTGT
CACCTTTGAGGTTTCTCTTTAAGCAGCCTGCTGAAGATCAGCTCTAGAGTCTTCAGCTCTAGAGTCTCCTGGACAGCTCTGTTCTCATCGGAGG
ACCTGGTTGAAGACAGCATCAGAGATGCTGTTACAACCAGCACTGAAGCTAACAATGAAGAGGAGCAGTGGCGAGTGAGAGACACCCAGAACTA
GACAATTCTGTGCAGCCTTCAACAGTCCACTCCCTGGGCACCAGTTCCCCTACTTGGAAATGAGAGGGATGAGGGCTCTATCTGTCTGGGTTTT
AACAGTAGAATGGTGGATATGTTCAAAGCAGGTGGAACTATCTGGCAGTGTTTTCACCTCTGTGGGGCTCAGCCTGTGTTTCTGATTCTCTCCT
TGGGAATGGCAGTTAAGTGGCAACTTGGTAACACTACTGCTCTGGGATGGTCCCTGCTCTGGGATGGCCCTTGCTAGATCTCTGTGGTTGTGGT
TGGTGATGTGGGAGGCAGGAGGAAGTGGATGGAGACAAGCTGACCATTGGTTTGAAGGTCCAAGCTTTTAATCCCCTTCCCTACCTGTGAGGAG
AGCCAGGCCAAACTGACCAGGCCTTGATAGCCATTCCCCAGCCAGCCGTGCCCTCCCTGGTTGACATCATCTTTTTCTCCTCTCTAGCCCATAG
AAGCCAACGCAGCTTCCAGCCACCATTCGTGCGTATGACCTTGCACCCTGACTTCATTTACTGAAGATATGCAGAGTCACCATCAGGAATAAGG
AGAATTTGGGGTCTGAGGAAAATCATCCCAAGCTTGGGTGGGGTAAAAATACCCTGTGGATTGAACCCTAAAAAAGTTCCTCAAAAATGACATG
ACGTTTTTCTGCCAATTAAACTTCCCTAACCGCTTCCTAGAAAAGGCAGAAATTCAGAGAAGTTGTAAGGCACCAGAGGCCGACCAGATCACTA
GATTCTCCCCTTTCAGCCTTGGAAAAATCATCCACTTTATTAAGGGCATCTTTAAGACCCATGGACAGGAAAAGCCTCCAGACATTTGCCTACC
CATGAACTGGCCATCAGCCGACAGGGTTTCCCACAGCTGCACCCTGAGCTAGTGCAGCAGCCACAGCCTTCCCTGTGCCATCCTACCTGCTGCC
AAGTCAGTCTGCAGACACACAGCCTGGCAGGATGGACTGGTCAGAGGTTCAAAGTTGCTTGTGCAGAGTGTTTCCAGCTGCCATTGATTTGTTA
GGCGCCGTGTCAGAAGCAAGTCAAATGGTCGTGACTTGTAGTAGTAGATTAAATAAATTGTGGTGTTCCCAGACAGGAAAACCTCTGTCAGTCT
CAAAATACTTATAGGGACTGTATTAACTCACAGGGATCTATGTCCACAATGTATTTGCTATTGGTTCCCTAAAGTGCCTTTTAACATAATGGTA
CTTAGAAGGGCATCTCTACAGATATGTCCGGATAGGCACAGAGTGCCTGCCAACTATGCCTGCCAAAAGCCAGAGAGCAATTATCTGGAGGTTT
ACTCTTTACTGTCTTCTTTATAATATACTCTTTTTGTATCTTAAAATATTTTTTACAATGATACATTGTTTCATTTTGTGCCAGGGGTGGGGGT
GGGGAGCTATACTTTCAGTTTTAAAATTTGAGTTAAGTCAATGAGCTAAATCACTCATATTTGAGAGATGTGACCTGGGTTGGTTGCCCTATAA
GACTAAAAGCCACATAGAGGACTTCTTGTGTGCCAGCCAACGGTGTCTGAGGACCTTCTGGGATCAACTTGCGTTACCTGTACAGTAGCTCCAG
GAAGCAAGTCTTTTATGATCCACATTTGACAGATGATCAAATGGGGAATTAGAGAAGTCATTTGTCTAACATCTCTGGCTGACAAGGGGTGAAA
CAGAGCTCTATCTGGTCAAAGGCCTGCTTCCAAAGCAACGGCCACCATGCCGTATCTTTGGCTTACAGCATCAATTCACCGGTTCTGCATGCAT
GGGGCACGGAAGGGACCGAGAGCTGAGCAGGAGCTAGGGTCACGGGGTCACGGGTGGGGAGAAGGCAGAAGTGAAGGCTGTGGATTCAAACAGA
TGACCCTGCGGGTTCTTGGGGTCTTGTGGGCCTCCTGTTCCCTCGGTTCCCCCCACTTGTGCTTCCTCCGTCGTTAACAGGGCATCGTTTCAGG
CACTCCATTTAGAATTAATTAGTTGCTATGGCTACCTACCTAGTAGTGAGTCATGTGGTACCACTTCACTGCCCTACTGTGTCCACTAACACCT
TGTTCCAGGCCAAGATGTAGGGAGAAGAGGGATTCCTATGTGAGGGAAGTTCTGAGTGAATGTGTGTTTCATTATGGGCATAGCCTGTCAAGGA
CTCCAAGACCAACTGGCCCATGGGAAACTGGCAAGGCTTTCCTCGGGAGTCTAGGTATGAGTGTTTATGACGAGAGGTCAGTGTGTGCAGGGAT
TAGTAACTGCAGCTTCTTCCTCCTAGACGTTTACAGCCTGAGGCTAGTCATCTGCAGAGACCTTCTACCAGACTAGCCAGCACCTCCCCTGTGC
TGTACCTTATAAAGGTTGCAGGTACTGGCCATAGTGGTGGATTAGAGAACCCCGCCTGATCCCAGCTTCACAGTACACATATGTACTTTCTTCA
TTCCCTGGGTGCCCTTAGTCAGAGTTCTGCGACCCAAGCTGAGCCCATCACCCCCACACTGAGACACACAGACCCCAGGGTCTCTGGGTAGGGA
AGCTGTCCCAGAAGGCTGGAAGGTACACAAGCGTCCTCATCTCTCACCACCCTGTGGCAACGCTGTTCATTTGTCATTGTCTATTGGGCATAAG
CCCCTGAGGCTGACACAGTTGCCTTGTGGAGGCCTGTATCCCCAGCCTTGGAAGAGAACACTCAGTAAGAGAACATCCTCTGTTGATAATTGGA
AGTTATTCACAAGAGGCCTTGCAATTAAAGTGGCATTTGTGAATTAGCTCGTAAGATTTAAAAAAAATTTTTTTTCTGTTTTCTACTTACCTGG
CTCAGAGTTCTGACACCAAGAAGTTGCTGGTTTTTAATATGTACCTTAAACCAGAGGGGCCAGTAGTAGGCCCTATTTTATACTAGATACTCGA
GTGAAGAACTTCTAGGTGTTCTATGTACATTATAGGCTATAATTCACAAAGCCCTGTGTTGGAGGCCAGGGCTCTGAACAGTCAGGCCACACAG
CTAGAGAGGGGTGAAGACTGGCTTCTGTCTGGAGCTGTCCACACTAATTTCCACCCTGCTCCCTCCTCTTGAGTTGTTATAGCTTTGAAGAACA
GGCCTGTTCTTAACTCTGCCGCCTACACCCATGAGAGAGCAATTTAAATTTAAACGGCATTCTCCTCTTCTATTGCTGGATTGGAGACTACCAC
ATTCTTTCTTTCTCCATCTGCTGCTACTTCAGTCTTTCAACTAGAGGAGATGGGTGAAGGAAGGGGAGAGAGAGAGAGAGAGAGAGAGAGAGAG
AGAGAGAGAGAGAGAGAGAGAGAACTGCAATCAGAGTGTGGCCTGTACTGCAATCAAATTGCAAACATTTTCATCCAAAAATGGAGCCAGGTGC
CTTTTAACTGCCTCCCCCAGTGCTCTCCGACACTAAGCTGTAGTCCGGTATCCTCTGTCTCTGTGTGAGTTTGTCGACTCCGGATGCTTCATAG
ATACGGGATTATATAACGCATGGCTTAGTGCCTGGCATTGTGACCATAGAATGCTTTCAAGGTGCATTGACATCTCACGGTATCATATCATCAC
ATCATATGCCGCTGTGCCACTTATCCTGAGGACTCAATTATATTCCATCATACGGAGTACTGTGCTTTATGCAGTGCTGTTTGTGGTACTGTTT
GTGGGTACTTAGGTTGTTTCCACTTTTGGACTATGGTGAATAATGCTGCCATGAACACTGACGTAGACTGTGTGTGGATATACATTTTCACTTC
TCTTGTCATGTGTACATTGCTATGCCTAGAATCTTAGGTCATACTGGAGGCCTGTGCTATGGTCTGAGGACCTGACACAGTCAGTTCCAGCTAA
AATTCCAATCCTGTATCTATTCAACTCATGCCTTCTCCCAAAGCAAGCTAAGCTCCTGGCTTTCCCGCCTCAGAGCAGAAAAGAGACGTGGCCA
CCTTAGCTTGGTTCCAACTGCAATCAGTAGCCTTAGAATCCTGTCTTCCTATGCCACAGGCGTCCAGCCTTTCTGCATCTCCGTGATCCAGACC
TGGGAGCAAAGGTAGTCCCATTTCCTTCATCCTTATGAACACCACCATCTCCCATTACACGCATGCGCAGGGTGGTAAACTTCCCACCTTTAGA
AACTCCATGCACCAAACCTTTGTAATGCATCCAGTGTCAATGCATCCAGCATCCTGTGTCCAGAATCCTGTGTCATGTGTCCAATGTCCTGCGT
CCAGCAATCCTTTCTTCTGTCCAGGAAGAGGGGGAAAGAGGATGACACATGTCTTCTCTAGGCTTCTAGAAACTTCTACCTGTCACATAAATCC
ACTCCTCTATTTTTCATTGTTGAAGTCCAGGAGTCAAATGCCTGACTTCGTGCTTGACCTCTGACAACCCCTTCACCCCCAACCCCCCCACCCC
CGCTCTGAAGAGGCCTTTCTGGGGGAAGGAGATCTGACTTAAATTTGACAATTTTGAGTTCAGTAACTTGCCTTTTGGCCAGGAGTCAGAGCCT
TAGTTATTCTCTGTTCACGGTGCAGAGGGGTGAAGTTGGAGTGGCAAGTACTTCATAAAGCCCCACCCTTTGCTTTTAGAGCTCTTGTCTGTGA
ATGAGCTAAAACCTCTACTTGGGTCCTGAAATGAACATCTCAGCTGCTTTGGACAGAGCTTCAGTCTACCCTTCCCCCCACCTCCCTCCTTCAG
TGGGGATCCTGATGGAATTCCAAGCTTCTCTGTATCCTGAAACAGACCGGCCTCCAGTTCCTGGGCTGTAATACAAGGGGCCAGTTTCCAGGCA
GATTTCCCTGTCCCCAGCTGTGTATGTCCTTCTTTCTACCCACAAGTCCCTATGCTCCAGAGGAGGCTAGCCATTACTCCCTTCTATCAGGTTC
CAGATAAGCCCATACCTGAATCTTGGTTGTAGACTTTGGGAAAGGTGAGAGGACCTGTTCTAAAGCAGCTGCAGGCTTCTCTTAGGTCTCAAGC
CTTCCCCAGCCCCTGCCCTTTGTTATTTCTCCAAAGCCAGTCCAGTCAGAGTAGGCACAACACAACAGTCACCACAGTGCATTTAGAAGAAAAT
GTCTTAGGGAGAGACAGAAATGGGCGTGACATAGCCTCTCTTTACTCTGTCTCTGTTCATAATGGAAGCCCTTGGTAATTTTGTACACTTAATG
AGATCATTATTATGCATATTATAATAGTATGTTTTCTGACCTAATTTGTTGTTTTCCATTACCAATATATATACAATGTTAGAAGGATCTACTG
TCCTCTCTGTCTCTCTGTTTCTGTCTGTCTGTCTGTCTGTCTGTCTCGCTCCCTCCCTCCTTGTCAGAGACCTTTGGGAACAAAGAAAGAGCTT
ATGAGTGTTTATAATGTAAGAGATTTCACAGTTATTTGTAGTTTAATAAATAAAGACATAGGAGCCCTTTGCTAAGTTCTTAAAATGTTTTCTC
GGTTTTGTTTGATATCTAACCATAATCTCCATTACTCTTCTTTTAAAAGTTACTTCTATAGATTTAAAAGAGTAATATGGGTTCATAGCAGGCA
GAAAATGCAATATAAGGTGGAAAAAAAGAGCAACTGCATACCAACCACAAAGCCACCTTCTTGTCCCCCATCCCGAGAAACACTCTGTGGAGAG
TTCTGTAGCTGCCCCTCTGGCATTTTTTTTCTCTTTAGATACATAGCTTTCCAAGCAGGCCAGCATGCAGAGACGGCAGCTTGAGTTGCTTTGG
CTGTGTTGTAAAGACACTTGGGCTTTCCTTGTTTCAAACTAGTCCAGGAAAGGCTGGTGGGTGAGCAGCCACTGGAGCAAATTCAGGCCTAAAG
AGTGGTTCTTCTCCATGCTTCGCTCCTGGAGGGTGGGCTGAGGATCCCACAGATGCTAAAATCCCAGAGCCACACAGAGACTGCATCTCAGAGC
CACGAGTGGGAGGAGGTCTGCAGAGGAAGGCCTGAAGACCTCTTCCCAGACAGAGTCACCCTTATGAGCCCTCCTTTTTTGTAGTCGAGGCAAA
CACTCCTTTTTGCAATCATTACTTCTTGCTCAAGTGAGAGAGTAAAATAATATATATTTTTAAACATTTTAAAAGCAAGCAAACAAGTATAGAA
GAAGGGCCCTGCAAACCAGGAGTCAGAGAGTCAGGGAGGCAAAAGCAGAGAGCAAGAAAAGCCAAGACCTAAATCTCTCACATCCCCTACCTCT
CCTGCCCCTGAGGGTTCAAGAAGTCTTACCCTCCGCATCTGCACTTACTGTAGCCTTTATTTTAATCACCTTGTTTGAATTTTGCCCTGCTCAG
CTTCATGAAATGGTGGGGCCACATGGGCCTGAGTTCCAATTTAGCCATGCAGCTAAGAAACCACTGCTATATCTTGTGCTGTATGTTAGCCATT
CCTTCTTGTGTAGACTGACTTTCTAGATTAGAAGTGAAAATGAGATAAGATACTGCAGGGAATTAGTGCCAGGTCATGCCCACCTAGATGCTGT
AGTAAACTAATGCCCACCCCCACCCTCTCACCCCAAACCCTAGTCATAGTCTAGACCCAGAACTTCTGAATTTACTTGGAAACTAACAATTTAT
AGATACAATTATAATTGATCAGGCTGGACACAAATGCAATGGCTGATGCCTTTATAGAGATAGGAAAGGGACAGAGATATATGTACAAGGGATG
GGGCCATGTAAAAATGGGGTGGTGCTCTACAAAGCAAGGGATGCCCAAGACTGCCATGTAGCTACCTGGTGGGAAGCCATGGAGAAGTTGGGAA
GTGTTCTTGTTGTATTTATTTTCTCTATTTCTCTGTGTGTGTGTGCATGTGGGCATACCTTTGTGTGATGCACTTGTGAGTGTGAATATGGAGA
CCCGAGGGTAATGTCAGAGAATCATATTCCACCTTATTCAATGAGGCCAGGTCTCTCAGTCAGACCCGGAGCTTGTGATAATGGCTAGTCTCAT
TAGCCAGCTTGCTGTGGGGAATCCCTGGTACATGTGGACCAGGGGTACAGCTAAAGGGCAGCTGACAAGAACAATGGCTCTAGGGTGCCTGGCA
GGTAAGCGGCTTGAGAGAGCCTAAAGATAAAACAGAACTGTTGAAAACAAAACCAAAGTATGCTTCCAGGCAGCATGCTCCAGTTCCTCAGAAA
CCATTCTGGTTTGAATGACACCCCACACCCCCGCTTTTTCTTTTTAACAGGAAAACAGCTTCAAGGATCGTGACCACATCATTTTTGTTCCCTT
TGTTGGATATATCCTAATGTCAAATGTGGCATATCTTTGTTGTCTCCTTCTGTCTCCCAACTAGAGAGAACACACTTACGGCTCCTGTCCCGGG
CAGGTTTGGTTGTCGGTGTGATTGGCTTCCAGGGAACCTGATACAAGGAGCAACTGTGTGCTGCCTTTTCTGTGTCTTTGCTTGAGGAGCTGTG
CTGGGTGCTGATGTGAGTATGAAGTACATTGGGAACTTCTCTTACTTTCTGGGTTTGTGTTGTTGAGATGGGTGAGTCTGTTGTGCTGGGGGAA
CACTTTTTAAAAATCCTACTCTGAGAAATTGTGCTTACAATGAAATCATTCCTACCTTCCAAAGACCAGGTGTTTCAGTGTTTTTTATTCTTTT
TTTTATTAGGTATTTTCCTCGTTTACATTTTCAATGCTATGCCCAAAAGTCCCCCATACCCACCCCCCCCAATCCCCTACCCACCCACTCCCCC
TTTTTGGCCCTGGGGTTCCCCTGTACTGGGGCATATAAAGTTTGCAAGTCCAATGGGCCTCTCTTTGCAGGATGGCCGACAAGGCCATCTTTTG
ATACATATGCAGCTAGAGACAAGAGCTCCGGGGTACTGGTTAGTTCATATTGTTGTTCCACCTATGGGGTTGCAGTTCCCTTTAGCTCCTTGGG
TAATTTCTCTAGCTCCTCCATTGGGGGCTGTGTGACCCATCCAATAGCTGACTGTGATCATCCACTTCTGTGTTTGCTAGGCCCTGGCATAGTC
TCACAAGAGACAGCTATATCTGGGTCCTTTCAGCAAAATCTTGCTAGTGTATACAATGGTGTCAGCGTTTGGAAGCTGATTATGGGATGGATCC
CTGCATATGGCAATCACTAGATGGTCCATCCTTTTGTCACAGCTCCAAATTTTGTCTCTGTAACTCCTTCCATGGGTGTTTTGTTCCCATTTCT
AAGAAGGGGCAAAGTGTTCACACTTTGGTCTTCGTTCTTCTTGAATTTCATGCGTTTAGCAAATTGTATCTTATATCTTGGGTATCCTAGGTTT
TGGGCTAATATCCACTTATCAGCGAGTACATATTGTGTGAGTTCCTTTGTGATTGGGTTACCTCACTCAGGATGATGTCCTCCAGGTCCATCCA
TTTGCCTAGGAATTTCATAAATTCATTCTTTTTAATAGCTGAGTAGTATTCCATTGTGTAAATGTATCACATTTTCTGTATCCATTCCTCTGTT
GAGGGGCATCTGGATTCTTTCCAGCTTCTGGCTATTATAAATAAGGCTGCTATGAACATAGTGGAGCATGTGTCCTTACCGGTTGGGACATCTT
CTGGATATATGCCCAGGAGAGGTATTGCGGGATCCTCCAGTAGTACTATGTCCAATTTTCTGAGGAACCGCCAGACTGATTTCCAGAGTGGTTG
TACAAGCTTGCAATCCCACCAACAATGGAGGAGTGTTCCTCTTTCTCCACATCCTCGCCAGCATCTGCTGTCACCTGAATTTTTGATCTTAGCC
ATTCTGACTGGAGTGAAGTGGAATCTCAGGGTTGTTTTTTATTCTTAATGAGCGTCTTCATGAGGTAAAGAACTTTCTGCATTAAAAAAAAGTT
TCACTATAGAGAAAACTATTCTTGTTTGGGGCTTAAATTTGTTTTTATTAATATTGTACAGTGGTGGTGGTGGTGTGTGCCATAGCACATGTGT
TGACCTCAAAGGACAACTTTGTGGAATTGGGTCTGTCTTTCTGCCTGTACTTTGTAGGGATGGAACTTAGACTTGGAAGGCCTTGACCCTTACC
ACCAAGCCATCTCAACAGCCTGAGGGGGGAAAAAACATTCTTAACATTGCCTCCCTCTGAGTTTTGAAGTTAACAGTTTAATAACTTTGAAACT
CTACTTTTCTACATCTGACATCTGTGTGTGGTTATCCTTTTGGACAGCAGCCAATATTCCCATCTGGCCTGTGTTTACAAAGCAAGACTCTGAG
AGTACACTATTAGAGAAAGAAAGAAACATGTTTAAATGATAATATTCCATGGTTCTAAACAATTTTTTATTTTTCTTCATTTTTAAAAAAATTT
TAAAATTATAATATAATTACATCATTTCCCCCCCCCTTCCCCAAAATCCTGTACATCTTCTGCCTCGCTCTCTTTCAAATTTGAGGCATTTTTT
TCATTAGCTATGGTTATATATATTTCTAAATACAACCTGCTGTGTGTGTGTATAGTTTTACTCATGTGTACGTTTTCCGAGCTGGCCATTTGGT
ATTGGATAACCAGTTGGCACAGTCCCAGCTTTCCGTAGTTGCGTATAGTTCATGGTGTAGGGATGAAGCCCTGAGGGTTTTCTCCTTTCTCCTT
TGTCTCTTGTTATTGTCTTTCTTCAGCTCACATTTTGGCAGTTATGCTACTGAGACTTTATGGGTGTAAATTCTGACATTACTAGGAAATACAG
TCTCACAGCAAACACCCTGGTCATCTGGCTTTTGTGATCTTTCTGCTCCATTTTCTCCCCTTCCAAGTATGAGGGCCCATTAACAATGAACATT
GCTCTTGCAATGGAAAACTTGCTCTACTTACCTTTATTACCTGAGCTCTTTTCATCAACAGACTCTCTTCCTTGTCTGATGCTTCTGAGTGTCT
TCTCTTCTGTCTCCATCTGCAAGTCATTCCATTGGCTTGGAATGCTTGAAGATTCTCTGAGCCCTGAGTGCACTGGATAGTGCCTGGCTGCAGT
AGCCATTCTTGTTTGTCAACTTGACTCCCATCTGGCGTTAACTAAAACCTTAAAATGGAGGGATTTTTGCTTACTTTGAAGTGGGAAGATCTAC
TTCTAATCTGGATCTTTGAGGTAGGAAGACACACCTTTAATCCAGATTTTAATTCTCATCAAACCTGGGCCATGCCTTCTCTTCTATTAGAAGC
CTATACAAGACATGAAGAAGAAAGCTTTTGCTCTTTGCCTGCTTGCTCTCACCTCTACTGGCAACCTATATAAGGACATGGAAGAAGGAAGTTT
TCCTAGCAATTCTATTTCTTCACTAGCATTAGGGCCTACTTCTTCAGAATTCCAAGGTGTACTGAAAACCATCTAAGTCAACCAGCGTTGTGAA
CTCATAGATTCTTTGGACATTCCATTTTTATCCAGACATTGTTGGATTATCTGGATCACAACCTGCCTGCAAGCCATATATATATATATGGAGA
GAGAGGGAGGGAGAGAGAGAAAGAGAGGGAGAGAGAGAAAGAGAGGGAGAGAGAGAAAGAGAGGGAGAGAGAGAGAGAAATATTCATTTTCTAA
GTTCTGTTACTTTAGAGAACATTGACTAATACATTGGCCATGACCACAAGGATGCATTGAATGCTGTTATATTACTGTTTTAATATTTTTTTTC
AGTAAAATGGTAGGCATACCACCAGAAATAAAGTGGAATATAAAACAAGAAGTTTGATTCTGATTTGAAAAGAAGATACTGGAAATGTCCCCAG
ATATTACTGAGGTACCACAAACTGAGGTACCACGCTTTAGAGTTGATTAGCCTTACAAGATAGTTTCAGTGTGTTCTTTTACTAAGACAGTATC
TTCAGTCCCCAATATTAGCTAACACTTCCACGGTGGAACCGACATCTTTGTCTAGCCCTAAAACCTTCTGACCTTAAAGCCTCAGTCATCCTTG
ATGCTTCTCTTTTTCTCACCTATATGGCTGATCAGTTGCCAATTGTTTCTTCAAGTGTCCTGTGAATGGTCTTACTGTCTCTCCTTTCTCTCTC
TTGCTTCCCCTTTCTGACTCTAAACAAAGATTTATGATGAAACATCTAGAAGACATTGATTCACTGAGTGGTAAACCTCCATTATACCCCTTCT
CCAGTTACACACACACACGGACACACACACACACTGACACACACACACACTGACACACATACTGACACACACACACACACTGACACACATACTG
ACACACACACACACTGGCACACACACATACACTGACACACACACACACTGACACACATACTGACACACACACACACTGACACACACACACACAC
TGACACACATACTGACACACACACACACACACACACACTGACACACACACACACATACTGACACACACACATACACTGACACACACACACAATG
ACACACACACTGACACACACACTTATACACACTTACACACACACACTTTTCACACTGTTTTGGCATTTTAACTGTTTTGGGATGAAGGGCACTG
CAACCTCATTATCCCTCTGCCTGGCACTAAGCACATTGTTGTTTGAATTTGAATTTCAGAAACCACTTTGTCTGTTGGCACTGCCCCTTCAGCT
TTCCCATGCCTTTGCATTTCTGAGGCTTTTTATCTGAGCCTCTCAGGTGCCTACTCAAAGAACTTTCAGGTCAAATGTCTCAGAGCTATAAAAG
CCACTTTAAGTCCCCCATCCCTAGGAAGTCTCCATATCTGTAGTCAGTTCACATGATGCTGGCTCTCTTAACACTCATGTTACACCAGCTTCTA
CCTGAGTGGGGTTTTTGACCTTCTAACTTATTATAATGTCTTTAGCTTGCACGGGTCTTTTCCTAAGTGGATCCTCTTACAAGTTCTCCACAGT
CTGATAGAAAGACTCAAAAAAGCCAGTATGTATATTGGAGGAGCTTGGTGTTTATGGGATGGACAGTTGAGATGTAGTTTGAACTTAAGTATTC
CATCCCATTAATAGAGATGAATACAAATTTAATAAGTTGTCTGTTGTAATAATATAATATCTGTCACCACAGATGATGGGCAGATATAAAGTCA
TGCAATTTCTAAACCTAAGACTGTCTGTTACCTCAGAAGTGATAATGGCAGTCAATGTGAAGAATAATTGATCATTTACAATGTATCATCCAAG
CACCCACAAGCACTTCTGTACACCTCCAGACTCCCCCATCACTCCATACACCCCAGACTCCCCATCACTCCATACATCCCAGACTCCCCAGACC
CTGCTTGCCTAGCTACAAAGCAGTCTGTTCTTCCTTCTATTCACTGTACTTGACTTTTGCTTTCCTAACTATAAGGGCTCTAAGTACATATTCG
TTTAGAGGAAAAAAAATTGGTTCTCACTAAGAACTCCAAGGACAAAGGTCATATTTGCATTGCAAAGAGTTTTGAGACAAGTTTATGATGGAGG
AGCTTAAGGTATCTGTGCTGGCAAGCTTGCAAGCATTGAAAAGGAATCACTCTCCACCAATGGAAACTCTTGCTTCCAAGTGGGCCTTTGTCTT
AAGTAAGTCTTAGGATAATGATTGTCTACTTTGTGACTAGCTCATCCTGTTGTGGCCTTAACTTATAGGAAGTTCTTTCTCAAGTTTCTTTTTA
TCTAAAAGAAAAGGAATCCAACGGGGAGCCAGACTCATGGGTAAAAAATTGTGCGTGTCATTTATTATCTATCTAACAAACTTCTGAGACCTTA
CATGCCAGTAATGAGCTGTAAGTGATCATTATAGGTGACTAGGATACACTCAGGATACAGGAAACCACATAAGGCAACTAGAAAAGGGTTGTAA
TGAATGTAAAAGAGTTTAAGGCTTTTAACAAGACATATATGATGCCATACTAATCTTTAAAAACTTACAAAGAAACTGAGGTAAAGTTAACTTA
CAAAGAAACTGAGATAAAGTTAAAAAAATTTATTTTTACAAAGTAAAATGAGATTAGATTTTCGCAACCACCATGAGTTCCTGTTCATGGAAAG
GGCATGGGTCATTATGTGACCGAGAGGTAAGAGAGACCAGGGCAGGAAAAGCATTAAAGGCTGTCCTAAGAAAAACTTGACTTGAAAACAATAG
CACTTGTGTTTTAAAATAATTACCCTGGCTGCTGTATTGGGAATCAAATGTCAGATAATGAGATTAGGTAGGGTCTTGGAATCCTACTATCAGA
AAGGTGCAGAAATCACCACAGGAGATGATAATGTCCCAGATGGTACGGAATCTCTGGGGTGGTGAGAAGTGATTAGGCTCTGAAGATCAGTGAA
GAGACAGCCTATAGGACTAGCTGATGAATCAGACGCAGGGTGAGAGAAGAGTGAAGAATGGTTCCAGGATTGATGCTCTCTGCCTCAACAAGGC
TGGTGTAGAGGGAGCCACAAAGGGGGAGCCAGCAGTGAGGAAGATGCACTAGGGTAGAATGAGAAGCAATGAGAATCAGAATTATGGTGATGCC
ACAGGAAAGCAGAAGGGACAGGGACCATCTAAGATGCACAGTGGGCGTAGAAGCAAGAGGGTGGGAGGAAAGTATGGAATGACTCCTAATATTC
TGGCATGTGGTGGTGGTGGTGGTAGTAGTAGTAGAGTAGTAGTAGTAGTAGTAGTAGTAGTAGTAGTAGTAGTAGTAGTAGTAGTAGTAGTAGT
AGTAGAGTAGTCCCCTTGGGTGGCTAAAGAAGAAGGGAAGCTGAGCCAGTGGTGAGTGGCCCAGGTAGCCTATTCATCACTAGAACTCTGGCTC
TTGTCACCCTGTCACTGTCCAAAAGTGAGAACGTTGGCAAGTGCCAACTACTGTGCGTGAAGACAATAACTTTTCAAGTTATGCATTTTGTAAG
GAAGACTTACATGGTCCAGACATGTTGCAATCAGTACCTCAGGTCTGTTGCTTCCCACTTTGTTCTTCCCATCTCTCATCCCTGATGCCATGCA
GGCTGTGGCCTACGTGACCATTAAGCAGGTCCAATCTCTACCTCCAGCTTTATACTGTCTGCCCTTCCACATCTTGAGGAACTTCTCTGGGCAC
TCTAGCAGGCATTTCAGATCTAATGGATGTTCCTAGCCCTCTAGTGTCCTAATGACTCCATCCCTAAGCCCAGGAGGTCTGTATTTCCTTTTAA
TTACCTATACTGTCAGCAATGTCCTACAGGTGTTTTTTTGTTTTTTTTTTGTTTTTGTTGTTGTTTTGTTTTGTTTTCAAAAACATAGCTCTAA
GAAAATGGGCTAGTCTATAACAGATCAGGCTTTATTGCTCACACACTTCGCTGGGTTCTCTCCTCAACCTTATCTGAAATACCGGATGTGGCTG
ACATCCTGTAGTTCAGTAAGTGGAGACTGAAGAGCCTAGAAATGTTAAGAGAGAATCTCTTCCTTCAAAAGAAACAAGCAAACAGACGGGAGTG
GGCAGATGGTGAGGCAAGTGCCACCAACAGGCAGTGAGCGCTCAGCAAACATCACCTTCTGACCTTACTTGGTTACTCTGTTTAGCTTTGTAAA
ATGTGAAGGGGTTGGACAAAGTTTAAAAGACTCGGTTCTCCCTTTCACAGCGGTAACAATTGGCAACAAAGTTCTGGCTGGCTCGGCTTGCCTC
TCACATCCACAGGTCTGACTGGCAGGAAAGAGAAAGAACGTCCTGCTGGTGAGCGAAGAAGCTGGATTTGGAAAGTGGTGAGACCTGGAGTAAA
ATCCAAGGTCACCCTCTGTGTCCTGCAGGCTGCAGCGTGTCCTATAATCAGAGTGCCTTTTTTTTTCAAATTATTTTAATTATTAGAATGTGTA
ACTATTAGAATGTAAAGCTTTTAAGTATGCAAATAAAAAGTTTAAAACCTTGGTATGGTGAGATAGCCTAGTGAGTCTAGATTCTTATTGAGCC
AGCCTGGTGACCTAAGATCTATTCCTAAACCTCACACAACATGAAAGGAGAGCACCAACTTCATAAAGCTAGCTTCCAGATGCATACCATGGCA
CTTGCTCCTACACATGGCTTTGTTGCCAATAATAATAATTTTTTTTAATATTTCAGAATCTGAAAAAGAAAAGAAAACTGAAAGACTGCTTTTA
GAGTTTGAAAGACAACATTGTTGAGATAATTATGGAACTGCTGAGTCAATGTAATTCCTAGCAAAGCACACCAGTTCCTTTTGTGCAGAATAGA
CAAGTCTACCCTAAAATTCATACAGAAACTCAAGAGAGAGAAATTCAAGGGAAAGGAGGAGGAAGGGCCAGAGGGAGGGGGAGAGAGAGGACTT
CAGAAGGAACAAAGCTGACGGCCTTACTTCCTAATTTCAAACTTTTCACAAAGCTACAGTAAACGGCACTATGTGATGGGGGCAGAGAACAGAC
ACAGACCTGAGCAACATGGATGCAAAGTCCAGGAATACACCCCACGTTTATCATCCAGGATGCTGGGATAATTCTACAGAGAAAGGACAGTTTA
GTTTTTTAAAATAAATTTATTGGAGTATGTGCAAAAGAACCTTGCTCATACACAAAAATGAACTCAAAACAGATTAGAGACCCAGATATGAACA
GAACAGACTTAGTTCTTATGAGAAAATGTTAGCAAGTAATAAACACATTTGGCTTATTATACAATGCAATATCTTCCCATTATGAAAGGAATAA
AGTATTGATGTGTGCTGCACAACAGATAAACCTTACATCATGCTCAGTGCAAGAAGCAAGCCACAAAAGGTCAAGGGCTACTCAATGTCATTTC
TATGAAATGTCAACACAAGCAGTTGACCAACACAGATGGACACCTGCCAGGATTCTGGAAAAAGGAAGGGGAAATGGGACTATTTCTGAGTGAA
TATGGTTCCTTCTTTTAGTGGTAATTAAGATTTGCCAAATTAAATAGTGGTGTTGGTTAGCCAACTCTATGCATATACTAAGCAGTCCAACTGA
ATGAATAGTGTGCCAAGAGTTTACATTAAGTTTATACGGCCTGCTTTTGTGTGTTAACAACATTTTAATAAAGCTGCTGAACATTTAAAAATAA
ATTTAATGAACACATCTTTAAAACCCTCCACAGATCACGCCCTCCACTAGCCCAGCACCCTAGTGCAAATGTATACAGTGCCCTCCTAAGGCTG
CGTTGCAGCTCATCCTTCCAAAGCAGTCTACTCTCTAACCAAAGCACCGTCCTCTCTAAAAATGCAGACAAAGTCCCCGGGGCTCCAGAGCGAA
GCCCTCCACCGGCTTACTGGCTCTGCTTTCCCGGGCCTGAGGCGTGCAGCTTACTCTGGCTAATAAAGCCTCAAGACAGAAGGGTTGACAGCAG
TGTGTCTTCCCAGCTGGCTTCAAACATGAGCACTCTGTGGATGCAGAAAACCTGAGGTCAGAAGTGAAACAGGAGATGGAGCCACACTGAGCTA
GAGCAGGGCCTGCCAGTCATACCTCCTGTCCCCTAGAGCAGGGCCTACCACACCTCCTGACCCCGGCAGACCTGAGAGCTCTCATAAAACACAG
ACACCCACCCAATCAAAATGGCTATCGTCAAGGAAACATCAAAACAACAGATATTGGCAAAGATGCAGGCAAAATTAACCCTTGCACACTGCTG
ATGGGAGTGTAAATTAGTACAGCCACTATGGAAATCAGCATGGATATTCCTTAATAAAACAACTAAAACTGGCACCACCATATGATCCAGCTGT
TCCCCTCCTCCAAGTATACCCAGTGAAGTCTACGTCAGCTTCCAAATATTCATATATCCATGCTTATTGCTCTACCATTTATAGTAAGCAAGTT
ATGTTGCTGACCCAGGTGCTCACTGATAGATAAATGGAATATATATACATACATATGTATATATGCATATATGTATATATACATATATATGCAT
ATACATATATATACATATATATGCATATACATATATATATATATATATGAATGATGATAGAACATTAGTCAGCCATAAGGAAGAATTCAATGCC
ATTTATAGAATTGCTAATGAGGAAGGGGGCAATGAGGAGATGGGGGAAGTGGCAGATCCAGTAAATGGACTAAACATAACCCAAAGTACATTAT
AAACATACCTGAAGAGTTACAATGAAATCCATTGTGTAAGACAATTAACAGTTAGCTGATAAAAGTCTTTATAACGTGGAGGTAAACATTTTCA
GCATACAGACTCTACTAAGCTTCTTTGAGGTATATCTTAGAACAAGTAATAAAAATTAACCTCAATTTAATATGTCCAAAAGAATAAAAAGTGG
GTCAGTCACGGTGACACTTGCCTGTAATCCTAGTACTTGAAAGTGGAAGCAAGATCAGGAGTTCAAAGTCAGCCTATGCTATGTAACATAGATA
GAGGCCAGCCTAGACAATCTGAGACCCTGCTTCAACCCCCCCTCCAAGAAATAAGTAATTTGAAAGCTAATAATTAGAAAGCTCATTAAGAGAC
AAACCATACAACAGGCACGTTGGGGGTGGCAGCACTCAACAGCTTCATGCTTTCTGCTTAGTCAATTAATACATATTGATACACCAGCTCTGGT
CTCCTGAGCCCTGTTCTCCGGGATCACGCTCCGCGAGTTCCAGTCCAGCTACAGATGATGGCTTATTATTAGGTAAACAAGACTGATTTTGGTA
CTGACTGAGCTTTAGAAAGTGACGTGGAGGCCCGCAAGGATACGGATGATTGCCAGTGTGGGTGGCTGTTGCTGTCCACCCTGCTCTCCAGAAA
AGGCCATGCGAGGAGCAAGAAGAGCAGCTCATTTGAATTCAGAAAGAGTGTCGTCTTCCTCCTTGCTGTGCATAATAAGCTCTTCTCCAGAAAA
GCAACCCGCAGCCTCTCTTCTTGTCTTTGCTCCTCCATTGTCACTTGCATCTCCCATAAGATTGGATTCTGAGTCATTACCCTGCTCTTGGGAT
GAGTTAAACTCTCAGAGACTGAAAAAGCGCGCGCGGAGCCCTGAGACTCTCAATTGTCTGTTTCAGATTGACACAGACCATGCTGCTCCTGCTG
CCGATACTGAACCTGAGCTTACAACTTCATCCTGTAGCAGGTAGGAACGGAAGATGGGAGGGTCCTGGGGACGATGAGCAGGTGGTGGCTCCTA
GCCAAAGCCGGAAATGAGTATCTGTGGGCATTCTTTTAGAGGGAGTGTGGTTACTGGATATTTCAAGTAGCGAAAAGAATACTTTCCAGGGTAT
TATTGATCCATCTCTCCCCACTACCCAATGCTTGCAATGAATTACATGTGTCTGAACGAACTAAGAGGCCGCTTCCTTTAATGTAAGGTGCCAA
GCAGCTACCCTTTTGCCCTCTATCAGGGACAGTGACTGAGGCAAACATAGAGCACTTCGTTACTCATGTGAGTTTTTTTTAACAAGCAATGGCG
GCAGCCACTGGTCATTTAATACCATAACGTAGACTTGAATTGGAAACTCGGAGACAATAGACCCCCAATGAAAACTCAGTCAGAAGGCTATGCC
TTTGAACTTGATGGGACATACCGTGGAGGATGTGGTTTAGAAGGGAGAAAGTTGTTTCAGAATTGATTATTTCCCTGGGGAAGGCATTTCAGAT
CACTGGTGTGTGCCATCAGGGCTGCAGTACCAAGAGAACATTGGGTTTAAAAAAAAAAAAAAAAAAAAAGCAACAGTTGTGGGGCTAAGGGTGT
GGCTGCTCCACAGCAGGAGCGTGAGGCTTTCCTTGAGGGGAATCATGGGAAAGTGGTACTTTGGCGTTGGATAGCCTTTTGATAAGTGGCTGTC
TCCTCTCCCATCTCATTCTGTCCGGGGGGTCAGCCCACGCTTCTCTTCCACGCTTCTCATTCTTTCCCTGTGGGCTGCTTAGGAAATGAAGGAG
CTAGGGAAACAGGGATGGCAAGTTTTGGTTTTGTTGTTTTAATATTTTTGTTTTTTGTTGCGCGCCCCGCCGCGCCACCCCCCCCCCCCAACAC
ACACACACACACACACACACACACACACACACACACACACACACACACACACACTGGCACTCTCTGTAGGGGAAGGACTGTTTCTTTTAAGAAA
AGCAGCACTAGGCAATCACAGTCTTTATGAGCTCTGACGTATGTTGAGGGAGCGGGAAGGATGGTGGAAGCAAGGTATGCTTACATTCACACAA
GGACGGATATGTGGCAATGTGCAAGGCAGCTTATGTCTCATTCAGTCTTCACAACACTCATTTGGCAAGGAGAGAGGCTGAGACATGGGAAGGT
GTTTCAATGTGCCCGAGATGACATAGTTAGTTACTAGGTGGCATATGAAAGATTTTCACACGCTGATATCCTTTCTGCCCTTCCCCTGCCCCTA
TTATAGTGCTAACAGTACTAAATAAATTATGACAGTAGACAATAAATTGTGAATCGTCTAACTGGGGGTGGGATTTTTACATAAGTTTTAAAAT
GGAGATATATATTTTTCTGTTTTCCAATTAGATATTTTCTTTATTTATATTTCAAATTTTGTCTCCTTTCCTCTCCAAAAACCTCTCTATCCCC
CCCCCCCATGCTAACCCACCCACTCCCTGTCCTGCCATTCTCCTACACTGGGGCATTGAGCCTTCACAAGGTCAAGGGCCTCTCTTCTCATTGA
TGTCCCACAAGGCTGTCCCTCTGCTACATATGCGGCTGAAGCCTTGAGTCCCTCCTTGTGCACTCTTGGTTGGTGGTTTAGTCCCTGGCAGCTC
TGGGGGTACTGGTTGGTTCATAATGTTGTTCCTCCTATGGGGCTGCAAACCCCTTCATTTCCTTGGGTCATTTCTCTAGCTTCTCCATTGGGAA
CTCTGTGCTCAGTCCAATGGTTGGCTATGAGCCTCCACTCCCGTACTTGTCAGGCACTGGCAGAGCCTCTCAGGAGACAGCTATATCAGGCTCC
TGTCACCAAGCACTTGTTGGCATCCACAGTAATGTCTGGGTTTGGGAACTGTATATGGGATGGATCCCCAGGTGGGACAGTCACTGACTGGCCT
TTCCTTCAGTTTCTGCTCCAAACTTTGTCTCTGTGTCTCCTCCCATGGGTATTTTGATCCCCCTTCTAAGAAGGACTAAAGTATCCACACTGTG
GTCTTCCTTCTTCTTGAGCTTCATATGAATCTGTGAATTGTATCTTGGGTATTCTGAACTTCTGGGCTAATAGCCACTTATCAGTGAGTGCATA
TCATGATTGTTCTTTTGTGATTGAGTTACCTCACTCAGGATGATATTTTCTAGTTCCATCCATTTGCCTAAGAACTTCCTGAATCAATCATTTT
TAATTGCTGAGTAGTACTCCATTGTGTAAATGTACCACATTTTCTTGTTCAAGGACATCTGGGTTCTTTCCAGCTTCTGGCTATTATAAATAAG
GCTGCTATGAACATAGTGAAACATGTGTCCTTATTACATGTTGGAGCATCTTCTGAGTATATGCCCAGGAGTGGTATAGCTGGGTCTGCAGGTA
GTACTATCTCCAATTTTCTGAGGAACTGCCAAACTGATTTCCATAGGGGTTGTACCAGCTTGCATTCCCACAGCAATAGAGGAGTGTTCCATAT
CCTTGCCAGCATCTGCTGTCACCTGAGTTTTTGATCTTAGCCATTCTGACTGGTGTGAGGTGGAATCTCAGGGTCATTTGATTTGCATTTCCCT
GATGACTAAGGATGTTGCACATTTCTTTAGGTGCTTCTCAGCCATTCGGTATTCTGGAGATATGTTCTAATGTGTATTCTGAGCATTTCACAAA
CCAAAACATATGTTATGAGTTGTGTTGATGAATTAATAAAAAGAAAACCCAAACAGCATGTTTTGCTCGCTCAGTAAACTTAGAATTCCTGTCT
ACTCTTTCTTTGCAAAACATTTTTATTAAAATGAGTGTTAAATTAGAACGCCTCCTTGTGTGTTTTAACCGCATCGTTATGTGTCTTTCTAATT
AAGGTATTTTGATTTCTTAGAAAGCTATTGGTGGGGCACCTAGAAAACAAAGCTTAGGAATGCTGGTGGTTATTCAATGGTGATTGAAAGGGAA
AAGGCCTGGGATAGGGAGGAAAAGGGAAACTACAGTATCATTGTGTGTTTGTTTAGGCTTTTAAATAGAAACTGCGCATTCAGGTATTATTTAC
AAAATACAAAGTACAAAAATTTAAAGAATATAGGAGAGTTTTTAAAAGATCCTGAGAGTAGGGGGGCTATGATACTCATTGCCTGAGAGGACTC
CCTGGTTATCACGGGGGCGGGAATGATGACCCCAATTCTGTAATTGCTTGGTGTGTAGCCCTTGACCCGTATCTTCCCTGAACAAAACAGTTCC
TACCCAGGAGTTATCCGGATATGATTCCTCATTACCCTCTATGCATAAATTTCTAGGTTTAAATTTTAAAAAATGCTTAGCGGATCATTTAGTC
GTTACAGACAGACAGTAGCATTCCTAATGCTCTAAAATAGAGAAATCACGGTCCACGGCAAGGATATGTTATTATAAGCATGGAAGTAAATACA
CAAACAAGCTGCTGCATCCGTAAAACACCCAGGAGGAAGGATGACACACAGCACAATGCCTTTTTCCTAAGTCTGCTGTTGGAGCATCCCTCAA
TAATTAGGTGAAGCTGAAACAAGCAAGACATCGAGAAGCACCGGCCTCAGTATCAGTTACCTCTGGAAGCAGTAGAGGTGTCATTAGTTATGCT
CTAGTTCTTGGATGAGGGGTGGGCTCCTGGATGCTCATTTTACTTACAGGAATTGCGTTAATAACTCTCCTATAAATCACCCTGCTGTAAGATA
TGCCACGGACACCGAAAACCACACAAAACAGATACAAACTTGACCTGACCAAACACTGCTGGAAATACCCCCGAGTGTTCATCGCCCCCCCACC
CCCAAGAACTACCCCTCTACAGCCCAAGCAGGCTGCTCCTCAAAGCCCTGCTCATGTCCTCATTGTTACGGCCCTGATCTTCATAAAAACAATT
GGCTACCGATGATTAAAAGAAATCTTTTTTTGGGGGGGGGGGGGTGTTTCGAGACAGGGTTTCTCTGTATAGCCCTGGCTGTCCTGGAACTCAC
TTTGTAGACCAGGCTGGCCTCAAACTCAGAAATCTGCCTGCCTCTGCCTCCCGAGTGCTGGGATTAAAGGCGTGTGCCACCATGTCCGGCTTTA
AAAGAAATCTCTTAAAGCATTCAGACAGTATACGTTCTTTGTAGATTCCAAGTCAGCAAATGGAAGCAACCCCAGGTTTTAAGCAACTATATAT
TTTACTGAACACACTATAGTCTATTTACCTAGTTGATGGGCATTGCAGCTATTTCAGTGTCTGGGCTAAGTGAACCCCTGCAAGACTGTAGAGA
AGCCTGAATTGTTCTGATTTCATGATCCATTAATTATCAACTCTTTCCTTATGTATATTCTGTAGGTATACTGTTGTGTTTTATTAAAAAAAGA
TAAAGAAGCCAGGGATATGTCTGATGGAGGTGTGGTATGGTGTGGGAGAAGGGGGTGCCTCTGTGGGCCCATGCTGAGACATCCCTTCCCTTTG
AGGGACCAGTCATACTATGGTATAGCATAGAATAGAGTTTATTCAGGGCATAGGGAGGGGAGTTGAGGGGGTAGTAGAGACAGAGAAAGGCAGA
GAGCGAAAGAGAGGAGGTGTGGGAGAGGTAGCAGGAGAGGGAGAGAGGAAGAAAGAGGAGGAGTAGAAGCCAGTCATGAACATATGGAGAGAAG
AGGGGGAGGGGAATGAGGAGAGAAAGCACAAGAGAGGGTAGAGAGGAAGAGAAGAGCAAGAGCAAGAGAGAGGAGGGGGCAAGCAGCCCCTTTT
ATAGTGAGTCAGGCACACCTGGCTGTTGCCAAGTAACTGTGGGGCAGAACCTGAAGAATACATTTTTATATTAAAAGTATTTAAGTTTATATTC
AGTAAAATTCACCTTTTCACTAAGGGAAAAATAACTGTGTATTTTGACAAATACATGGTCATGTAATGGCCTCTGCAATCAAAGTGGGATTAGA
TAGTCTTTATGTAATTACTTATTTTTTATTATTAATATACATTAGATTATATTGTTTAGTGGAATTCACTGTGATATGCCCACACATGTGCACA
GCATAGTCTGCAGACACTTGGCTATCTGCCTTCTTTCTCTTAGTGTAATGCATTTGAGGTTCATGCGTGTTGCAGTCTTTATCAATGGCTTATT
TCTGTTTGTTACTGAGCGGTGTGTGCTCTGTGATCCAGTCCCCAGTTGTAGGGCACTTGGGCTATTTCCCATTTCTGGTGAGTTTAAATAAAGT
TGCTATAGATGTTTGCAAATAGGTTTCTGCATGGACATGCATTTTGGACTTGTTAGCTATATTGAATTTCTAGACCCTGTGGCCATTATAAACA
AATGTATAGGAAATTCTTAGATTTCCATGGCAGTTGTACCAACTTTACACCGTCATCAGCAGTGTCTGAAAGTTTCAGTTGGCCCTGGTAGCAA
CATTGCCGTGTTGGACCTCTGTTACCCCCACAGATGCATGGAAATACCTTGCTGTGGTTGTAGCTTTCAGTTTTCCAGTGGCCAATGATGCTAA
GCACATTTTAATGTGCTTATTGTCTAATCGCGTATCTTCTTTGGTAAACTACGTATGGAAATCATCTAATAATTTTTAATTTGGGTTCTTCGTT
TTCTGGCAAAGCGTCGAGTATTCCTTATTTATCTGAGATATAAGTCTATTATAAGGTATATGGCTTATGGATATTATCTCTCAGCTTGTTTTCC
ATTCTTTGAACAAGGCTTCATGGACATACTCACTGTGGTCTGACTTAAGGGTGTCATGTTTCGTGTCCCACACTTTTGATCTCTCAGCAGCTCA
AATCGAAAGGATTCTTTCCTAACATCTCATAGCTGTTCACGCTACATTTAGGTCTGTAATCAAGTTTGAGCTAGTGTCTATACAGCATATGGGA
GTGCAAGTCAAAGACCTAACAATGTTCACCCACTGCGGCCCCACTGTTCTCCGTCATTTCTTCAGAGGGAAGCACCTTTGACTCTTTATCACAA
AGCAATAGTTCATAGTTGTGTGGGTCCACTGACCCTCCTTAAGCTAATTTTGAATCTTTGGTCCGTGGCATGTGCCCAGCTTTTTGTGAGTATC
CTCTTCTTTTGATTCTTGTAACTTTACAGTTAATTTACTTTTTGTGTCTTTTATTGCAAAATCTAAAACTATCTTGTAGTCCAGATGTATTGAA
CAGATAGTATTTATTTTTATATTTATGTCTTCGGCAGAAACAAACCCGTACTTTGTATCATGGAGGTTTCCCTCCTTTACTGCCCTTTATCTTC
CAGATGTGGACAGAAGCAGGTAGGGGTGGAGAGGGAGAATTTGGCCATGATGCTGCTTGAAGGACTCTGGTGCAGAGCACACCCCACTCATTCT
GATTGTCAATGACTCTTGATATGTATGAGTATGATGGCTAATATTGATAATTGGCTTGCGATTCAGAACCACCAAGAAAACATACCTGTAAGTG
TGTCTTTGAGGGTGTTTCCAGAAATGTTTAACTGAGCAAGAGAGACTCACCCTGAGCGTGGATGGCACTGTGCTGTGGGCTGCCATCTTGGACT
GAATAAAAGGGAGAGAGTAAGTTGAAATGAGTGTCCTGACTGCAGGCGCAGTGTGACTGCTCCTGCTGCCAGGCCTTTCCTGCTGTGATAGGCT
GTACCTCCTTGAACTGTGAGCCAAGGCAAGCCAAGCCCTTCCTTCCTCATGTTGCTTCTTGTCAGCTATTCCTCCACAGCAGCGATGCAAACAG
GAAATAAGACAGCAAAGGAATGGACCCTGGCCAGGGTAGGATTTCTACAGTGAAGGATGTCCTGTGAGATGCTCACACACAGAGCCACAGAGCA
TGAGTCTAGTTCACAGAACAGTTTTCTGAAGAGTAGGGACTTTGTGAAAAACCTCCCAGTCCCTTCTCATATTTTAAGTACACTGTATAGGAAG
GCAAAAAAGAAAAGTAAATTTCTGTTGTTCAATAGAAAAAAGTTCAAAATTAGCCTAACAAGGAAACTATCTTGGGAGAGAATGCAAATGATTT
GTTTTGTGTTTCCTGGAGATGTGACAGGTGCCAGCTGTCACCAAGGCCTGTTTTCAAAATGCTAAATTGACCTCAGGATCAAAAAAAAAGACTC
CCAAATTATCTAAGTTGGGTGTCCTTTGATCGCTTCGGAAGTTCAGGTCTAGATTGAAGCTTAATTTAGTTCCTGCACAGCAAATGAAATGTAA
GAGAAAACACAGTTACCTTACCACAAAGCCAAACGTTTACCTGCCTCAGTCTTAGGTGCTGTCTACTCTTGCTGGCAGTAGCTGGTAGAGTCGC
AGCCGAGTCATCAGAGCAGAGAGAGGAGAGCGAATTATAGCTAGAGATGAATTTGTGTTCCATTTCCATAGACACCACTCAGTGGCCTTAAGCA
AACTGTTCTTCTTAAAACCTGTCAAATACGAACAGTATCACCTACTAAGGAGGGATGACAGAGGACCAATCAGAGGATAGAAGAGTCCCTGGGT
GTTGGGACCAGTGTGTTTAATAAAGGTTAACTATCACTCTTTCCCCAGTTACACAGTGTCCTAAGTCTGCCTCACAGATCCTGGGTTTTAGGTT
TGGGGCAAGCCTGAGAGACTGTGTTTCTAATTAGTGCTCAGGTGACGCTGAAAGCAACGTTTCAGGTGGCAAGGTGGTCGTCGGTGTTTGCAAC
TGCAGATGTTTGATCTGCCTAGTTAGACTTATTCAATACTGTCTGCAAGTTAGAAGCTCCAGGGAAACGGCAAGACGAATGTCTGCTTACCCCA
ATAATAACTTCATCTAAAGAATGCGAATTAGGGTTTGGGCATTAATATTTTTTTCTAAGAAAAAAAAAAGTCTTTGCTATGGTCTATGGTCTAG
ATATATGTTCCTCAAAGGTTCCTGTTCCCTTATACCTCCAGGGCAGTGGTGTAAGCTTTAAGAGGTAGGGTCTAACAGAAGTTAATTAAATCCC
AAACGTCCACACTGATAAAAAGAGTCCCATCTGTCTTTGAAGAGTGGGTAGTTATAACGGGAAACTACCCCAAGAGTATGGCCATCCCTTTCTG
CTTCTTCACACAGGCAGGGCTGTGTGCTGGCGCCAAGCTTCTTGGAGCCCCCACCACCAGAATCTTGAACTTCAGTACACAGCACACAGGTATT
TTGTTAAAGCAACCATGAGTGGGCAGAGGCAGTGTCCCAGAAGGCTCCTGTGTCAGACTGGCTGAGGCCTTCTGCTTGGTTAGTCTAACCCGCT
GACAGCCAGCCCTGAGAAAGAATTGTGTACAGAATCCATTACAAGTGGTTTCATTTCCTCTGTGATTCACAAAAAAGCTGAAAGTTATAGAATT
TCAAACGTATGTTGCATCTCCTAAACTACCCATTTTGTGAGAAATCAAGCCCAAAGCTTTGCATGTCTTGGAAGCTATACCTGAGGTGAAGTTA
GTCTAAGTAACAAGTCTCAGTCATTGTGAAGTATTCCGATGCACGGTTTGTCAGGTCATTGTTCTTTCCTCTTGTAAATACTACCCCCAAGCCC
CCAAAAGTCACCTATCTGAAAGGTAAAATGTATTTCTAAAGCTCAAGGCATTTATTCTGCAAGGGGTGGGGGTGGAGTGGGGTTGAAAAAAAAA
GAGAAGCCATGTGTTTATAAAAGGTTGGGGCAGGGGATGAGGGAAGTTCTGCGGTAATGACAGGGGAGTTCCAGAGGAAGTTATGGGCTCTCTC
TCCTCTCTCATGAGAAAAAAAAGATAGAGCGGTAAACAGCTTAGCTGTTAACACATCTCCAAATCTACCCAGCCAGTTATCTTCCTTGCACACT
CCTCTTTCCCCTTCACCACCTCTCTCCTTGTTTTCCGTTTGGGCATCTGAAATTTAAAAGGGGAAACAGACTTTAGTAGTAGTAAATTCCTAAG
CTAAATGCTCAGAAAATAGCTTAGTATATCTTTGATGAGGGATGATTATGCTTCCCGCCCCCTAGGGAGCCGCAGAGTGGATTTGAAACAAAGT
ATCTTTTAGTTACAAGTGAGTGAAGGGTTTAGAGATGATTTCTGATTTAGTCACTTTTCTCCCTGGCTGGTACCTAAGTAACAGCCTGGCAGAA
GCGACCTTAGGGAGAAGGGTTCATTTTGTTTCACCATTGGAGAGGACACAGGAGTCCACAGTGGTTGGGAAGGCGCGGTAGCAGATGTGAGGCA
GCTGTCACATCAGGAAGCAGGAAGAAAGGGATGCTGACGCTCAGCTAACTTTCTCGGTTTTACTTAACCCAGGACCCCAGTCCATGAGATGCTG
TCACTCCCAGGCGGATCTTCCCTGCTCAGTTAAGACTCCTAGGTGTTTCAAAATCCCATCGAATTAAGACTTAATCATCACAGTTCTCCTAAGA
ATGGATCTAAACACTAAGTACATAAGAACTTGTTATTTCAGCATGCAAAGGACTCTACAGAATGGAATCATACAGGAATTCCTGAGCTACATCC
TTTTTTTTTTTACTAGTATGATCCGAGGGTCTTTTCAGCCTGATCCAGCAAGGACACGCATGATTTACATTGTTTTGCATAGAAGCATATACCT
TAGAGCCTTTCTCCTGGGCCCCATGTTACACAAACAAATGCTGGGCATGTGTGCATCTCCTCCACACTACAGATCTGTGTAAAATGTGCATTAA
TGTTCTAGTTGTAGTCAAAGCAGAGAATGGAGGTAATAAACTGTCCAGTCATTTTTGGTAAAGCTCATTTGTGAAAATCACTGTCTGGTATGCT
GGGTGGTAACCCACAGTTGCTGATTCGTGTGGTCTCTTGTGAAGTATGCCTCAGGGAGAAGTGAATGCAGGCAGGCTAGGAGTCTTCTCACCAC
GCTCCCCTTTCCCTCCCTGCTCCTCCAGAAGTGAGATTAATATTTTGGATACTTGAAAAATGCCAAAGCAAGCAATCTTATTCATTTCTAAGGT
TTGAAGGATAATGGAAGTTTGAATTAAAAGAGACTGGCAAGCTGAGTATGGCCACACCCTGGCCACCCCAGCACGCAGGAGGCTGAAACAGGAA
AGGTTTGAGTCCAAGACTATACAGAACTATGTAACAAGACCCTGTCTTATAACCAAAGATGATCCAAAAGTGAATTGCAATGTGAACATCAGCC
AGCAGGAAAACCAAAAGGTTTTTGGAGTCAGGCCAGGGAACAGACAACCTTCCTGGTTAATTAAAATATTCTGGTTTGCCACAGTTGTGGTGGG
CACCTAGAATCTGAGGACTCAGAAGGCTGAGGCAGGAGGAGGACGAGCTAACAGCCTGGGATACATAGTGAGACCCTGTCAGCGAGAGAAAGAA
AAGAGAGAAAGGAGAAAGGACGAAAGCGAAGGGAGAGAGGGAAGAGGGAAGAGAGGGAGGGAGGGACAGACTATTCTGGCCAATAAAGTATGAC
TGTGCATTCCCTACATGAGTACTACTTTCCCAAGAATTAACCATCACCTGAAAGAACATTTTCTGAGTCCCTGCTGGCTGGCAGACACGGGTGA
TACACGTGCTCCACCTTGGTCAGATTTTCCCACAGAACCAAGAGATGGATATTTACCTCATACAGACAGATTCATAGAAGCTAAAAGCCAGCCC
CTCCCCTGCTCCAAAGGTCCCTAACTAATCCTGGTATTGTTGGGATTTGAACTCAAGCTCACTAAATAAAAATATTTTTTTAAAAGCCCACAAA
TATTTGATGATCAGATTATTTTCTTGTATGAATGATAAAAGAGATCTATGGAGTATAATTCAATCCAATCTTTTGTGATGATTTAGAAATAATT
TCCATGTCTTACCTTACTACCATCATTGGGATACCCATTCATTACTCAAGGATGCAGCATACAGGTTCCCACAATTTCTACATGCAAACACTGC
CTAGGCTGTTTCTGCTGTTGCAGAGTTATTGTTTTTTTTTAATTAAGTATTTTCCTCATTCCCAAAAGTCCCCCGTACCCTCCCACCCACCCCC
ACTTTTTGGCCCTGGGGTTCCCCTGTACTGGGGGATATAAAGTTTGCAAGTTCAATGGGCCTCTCTTTCCAGTGATGGCCGACTAGGCCATCTT
GTGATACATATGCAGCTAGAGTCAAGAGCTCCAGGGTACTGGTTAGTTCATAATGTTGTTCCACCTATAGTTGCAGATCCCTTTAGCTCCTTGG
GTACTTTCTCTAGTTCCTCCATTGGGAGCCCTGTGATCCATCCATTAGCTGACTGTGAGCATCCACTTCTGTGTTTGCTAGGCCCCGGCATAGT
CTCACAAGAGACAGCTATATCTGGGTCCTTTCAGCAAAATCTTGCTAGTGTATGCAATGGTGTCAGCGTTTGGAAGCTGATTATGGGATGGATC
CCTGGATATGGCAGTCTCTAGATGGTCCATCCTTTCGTCACAGCTCCAAACTTTGTCTCTGTAACTCCTTCCATGGGTGTTTTGTTCCCAATTC
TAAGAAGGGGCACAGTGTCCACACTTTGGTCTTCGTTCTTCTTGAGTTTCATGCGTTTAGCAAATTGTATCTTATATCTTGGGTTTTTGGGCTA
ATATCCACTTATCAGTGAGGACATATTGTGTGAGTTCCTTTGTGATTGTGTTACCTCACTCAGGATGATGCCCTCCAGGTCCATCCATTTGCCT
AGGAATTTCATAAATTCATTCTTTTTAATAGCTGAGTAGTACTCCATTGTGTAAATGTACCACATTTTCTGTATCCATTCCTCTGTTGAGGGGC
ATCTGGATTCTTTCCAGCTTCTGGCTATTATAAATAAGGCTGCTATGAATATAGTAGAGCATGTGTCCTTCTTACCGGTTGGGACATCTTCTGG
ATATATGCCCAGGAGAGGTATTGCTGGATCCTCCGGTAGTACTATGTCCAATTTTCTGAGGAACTGCCAGACTGATTTCCAGAGTGGTTGTACA
AGCTTGCAATCCCACCAACAATGGAGGAGTGTTCCTCTTTCTCCACATCCTCGCCAGCATCTGCTGTCACCTGAATTTTTGATCTTAGCCATTC
TGACTGGTGCGAGGTGGAATCTCAGGGTTGTTTTTATTTGCATTTCCCTGATGATTACGGATGTAGAACATTTTTTCAGGTGCTTCTCAGCCAT
TCCGTGTTCCTCAGTTGAGAATTCTTTGTTTAGCTCTGAGCCCCATTTTTAATGAGGTTATCTGATTTTTTGGAATTCATCTTCTTGAGTTCTT
TGCATATATTGGATATTAATCCCCTAACTGATTTAGGATTGGTAAAAATCCTTTCCCAATCTGTTGGTGGCCTTTTTGTCTTATTGACAGTGTC
TTTTGCCTTACAGAAGCTTTGCAATTTTATGAGGTCCCATTTGTCGATTCTCGATCTTACAGCACAAGCCATTGCTGTTCTGTTCAGGAATTTT
CCCCCTGTGCCCATATCTTTGAGGCTTTCCCCCACTGTCTCCTCTATAAGTTTTAGTGTCTCTGGTTTTATGTGGAGTTCCTTAATCCACTTAG
ATTTGACCTTAGTACAAGGAGATAGAAATGGATCACTTTGCATTCTTCTACATGATAACCGCCAGTTGTGCCAGCACCATTTGTTGAAAATGCT
GTCTTTTTTCCACTGGATGTGTTTAGCTCCCTTGTCAAAGATCAAGTGACCGTAGGTGTGTGGATTCATCTCAGGGTCTTCAATTCTGTTCCAT
TGGTCTACTTGTCTGTCACTATACCACTACCATGCAGTTTTTATCACAATTGCTCTGTAGTACAGCTTTAGGTCAGGTATGGTGATTCCACCAG
AGGTTCTTTTATCCTTGAGAAGAGTTTTTGCTATCCTAGGTTTTTTTGTATTCCATATGAATTTCCAGATTGCCCTTTCTAATTCGTTGAAGAA
TTGAGTTGGAATTTTGATGGGGATTGCATTGAATCTGTAGATTGCTTTTGGCAAGATAGCTATTTTTACTATATTGATCCTGCCAATCCATGAG
CATGGGAAATCTTTCCATCTTCTGAGATCTTCTTTAATTTCTTTCTTCAGAGACTTGAAGTTCTTATCATACAGATCTTTCACTTCCTTAGTTA
GAGTCACGCCAAGGTATTTTATATTATTTGTGACTATTGAGAAGGGTGTTGTTTCCCTAATTTCTTTCTCAGCCTGTTTATCCTTTGTGTACAG
AAAGGCCATTGACTTGTTTGAGTTAATTTTATATCCAGCTACTTCACTGAAGCTGTTTTTCAGGCTTAGGGGTTCTCTGGTGGAATTTTTAGGG
TCACTTATATATACTGTCATATCATCTGCAAAAAGTGATATTTTGACTTCTTCCTTTCCAATTTGTATCCCCTTGATCTCCTTTTGTTGTCTAA
TTGCTCTGGCTAGGACTTCAAGTACAATGTTGAATAGGTAGGGAGGGAGTGGACAGCCTTGTCTAGTCCCTGATTTTAGTGGGATTTCTTCCAG
CTGTTGCAGAGTTATTTAAGTCAACTCATTGTTTAAAATACAAGCAAACAGGTTACTTTGTATAGGCAGAAACTCAGTAACACACTAGTATAAA
GGAGGCTAGGAACAAAAGCAAGCACTGAAAAATCAAGCAGTCCTACTAAGTTCTTGCCAATGCGAGTCATCTTCCAAGGAGCTCCCTACCTTTT
GCTAAAGAAGAGATGTTCATTCCAAAACAATAGAGTCGAGTTAGGAAGCTTGTAGGTGAAGGATTGGAATCAAATTGAGAAGGGAATAAATTTC
TCTACAAACAATTCACTTCCCTTCAGGCTTTGGTTTTGTATGACATGAAATTATCTCGCATGTCTCTGATGAGATTCATCTCAAATTTTGGAAA
ATACTGTGAATAGAAACTGCAGCTTTTCAGGTCAAACACCAATGGGCCCTAAATTGCTACCATATGAACTGTATTCCTAAGAATACAGCCAATG
TCTTTGAACAAAATTATAAAATCTTAATGACCATACATGTTTCAGGTCATAGTGTTAGGTAATACAGTAACAAAGTACCTTGCATGGAACTTGG
CCTGTAGTGGTTAAAAATAGCCCCCCCCCCCATCACCTTTGACTTTCTTGATCCAAGAGTGAACATCTTCACTGAACTTTTATGACTTGGTGAT
TAACAGCATGTGAAACCTTGAGAAATCCTGGACCAGTCTTAGCTATGCACTCTTAGGCAAGTAATTTAACCATTCTATGCCTTACTTCATTTTA
AAGGCGGTAACAATGCTGCCTGTCTCCCTGGCTTCTAGGGGGACTAAAAGGGATACTAATGTATCTATGAGCTCTGTGAACAATAAGAGGTGTT
GTTACTATCGCTGGTTGGAGGTGGTGATGTTTGGCAAAGAGAGGTAAAAATTGACTTCATACAAACTAAAGAGAGTCAGCCCAGGTGATTGAAA
TATAATGGTAATAAATAAGGCAGATCGCAGTGGTAGGGCAGCCTAAAGAAAGGTTTGGTCTCAAAAGAGGAAATGCTCTGGGTTGACTGCAGGG
CCCTGAGGAGCTAGCGGGAGATGTTGTTGAGGGCAAGCAGGCCTGTCCATCAGAAGTTCCTGCTGGGGTGTTCAGGAGAAACAGGAGTGCGATC
AGCTGTGCGGGTGGGAATGTTCAGATGAGAAAACCACCTCCAGGTCCCGAGGCAAGAACTCCACTGTTTCTATATGGGATGAGGGGAGGGAAGA
GAGGAGCCCAGAGAATGGCGAGGATCTTCTAAAAGATGCCGGAGGGAGGGAGCACAACTCTCATTCCAGAAGAACTGAAACGTTGACATAAAAG
CAGACAACAGCGAGCTGTGTCTCCCTTGCAGCTTTATTCACCGTGACAGCCCCTAAAGAAGTGTACACCGTAGACGTCGGCAGCAGTGTGAGCC
TGGAGTGCGATTTTGACCGCAGAGAATGCACTGAACTGGAAGGGATAAGAGCCAGTTTGCAGAAGGTAGAAAATGATACGTCTCTGCAAAGTGA
AAGAGCCACCCTGCTGGAGGAGCAGCTGCCCCTGGGAAAGGCTTTGTTCCACATCCCTAGTGTCCAAGTGAGAGATTCCGGGCAGTACCGTTGC
CTGGTCATCTGCGGGGCCGCCTGGGACTACAAGTACCTGACGGTGAAAGTCAAAGGTGAGTGTCCCCCGCAAGTCATCTCTGACGGAGGGCTGT
GATAGCTTACACAACAGCATGGGTAGCTCAGGCCAAAACCAGAGACCGGGTCTTAAGAGGCAGCCCCTTCAAAGGGGCACCTGAGCGGAAATGG
TTTTTGGCATCTGGTATAAGATCTAAGTCAAGGTAACGGGGGTTAAAGCTTACAATGGAGCCAGGGGAAGCCAGGAAAATGAGCAGACACCAGG
CCTCTTCCTCTGCTGGACTCGGAAAGTACCGGCTGAAATAACCTACTAATGGAGCCACATCTGTGCCTTCAGCTCCATTCCCAAGGGGAACTTG
GAAACCGGGGAATATGGCTACCTGGAGTCAAGAACATACTGTCCTGACTTTCCAAGAGGGGAAAGCTTTTGGAATTAAAGTCAGGCCCTATAAT
ATAATATCCATAAGCTGTTTAATGACAGATTGGAAGCAACCCTCAAAGATTTATTTCAGTGGTTGTCTTAGTCAGGGCTTCAAGTGCTGTGATG
AAACACCATGACAAAAAAGCAAACTAGGAAGTAAAGGGTTGACTCAGTTTACACTTCCCAGATCACAGTCCATCACTGGAGGCCATCAGGACAG
GAACTCAAGCAAGGCAGGAACTTGGAGACTGGAGGCCATAGAGGGTGCTACTTACTGCTCTGTCCCTCATGGCTTGCTTAGCCTGCCTTCTTAC
AGAACCCAGGACTACCAGCCCAGGGAAGGCACCACCCACCATGGGCTGGGCCCTCCCCCACTGATCATTAATTGAAAAAATACCTTACCGCTGG
ATCTCATGGAAGCATTTCCTCAACTGAGACTTCTTTCTCTGTAGCGACTCTAGCTTGTGTCAAGTTGACACACAAACCCAGCCGGTACAGTGAC
TATTTGAAATTCACCTGGATCCTGTTAAGACAAAAGCATATAAGAACATTTCCACTTTATTCTGAGCCACTATGATGATTAAGACAACAATTGC
TGTGTGCAAAGTTTGCCCTTGTCTTAGAAGTGATGTGGGGGCTGCCGAGTTTGCTAAGAGCATTTGCTGTGCAGGAGTAAGGATCTGAATTCAA
AGTCACATCACCTGCAGTAAAAACAATTTTTGTTTGTACCCTCAGTGTCCCAGAGCAGTAACAGGCAGGACCTGGGAGCTCCCTGGCCAGTCAG
CCTAGCCAAAACAGTGAACTTAAGCCCATCTCAAGAGAGTGCTGTGGAGAACAGTAAGGAAAGCCATCTTCTGATCCTTACATGGGCATGCCCA
CCTAGACACATATATATGCACAGAGAAATATACACCACATACTCCTTATATGGACACACACTCCTGCACACACATGTGCAGAGTAACATTCACC
ATACACCCTCCCCACACACATGCATACATACACCAGGGAAATAGAAACCATGAAGGATATAGAAATCTAAAGTAGAAATTAATCAATCTTTTAA
TGTTAGAGACTGAAAGCTCAGCAAGGGAACAAGGTAGCAAGTCACATCAGCAAGAGTACTAAGGAAGACGTGGGTGGTGGGCAGTGGAGCCCCT
GGGAATGTCACTTGGCAGACCGTTCAGTGAGAGCCGGTGACAGGAATGAAATGATTCTGCTGCTAGAGACACTGTCAGAGCAGAGCAGAGAGCT
AAGTCCTTTAATTAACAAGAAAGCTGAGAGTCTGAACGGTCATGTCGCCAGCCCTTGCCTAACACCTTTCAGATTCTTAGCCACTGCGCTTGTT
ATGTTATTTTACCAAGCCACACAAGTGTCTGAGGGAAGAAGTTGATGGCATGAGAATGTACTTCCCAATGGAAGTGTAATTACGTGGTGCTTAT
GGGAAGATGGCATCTGAAGGAAGAGGAAGCTCCCATTGATAGCTACATTTAACTACAAGTTGGTAGCTAGGAGATTGCTAGTATGAGAAAAACA
GATGTTGCTAAAGATAAAAGGCCATGTTTTCATTTGGCCTACTTTAGATTTCACTGTGCTCTAGCCATAGTGGAAATCAGACTAGAGGTTCCTT
AAAAAACTAAAAGTAGTACTATCGTTTAATTGAGCTATAGTACTCCTAAGTATAAATTCAAAGGACCCTAAGTCAGCCTACCATAAAAACACAC
ACCACATTTATTGTAGAACTATTCCTCTTAGGCAAGTCACAGTCTAGTTGGCCACAACAAATGAATGCATAAAGAAAATGTAGTGTGTATGTTT
AATGGAGAATTCGGCCATAATGAGGAAAATAACCATGTCCTTTATAGGAAAATGGATAGAACTGGAAATCCCTGTGTAAAGAGACATAATGAAG
ACTTGGGAAGAAATGTATAATGGTAATAGGATCTATGTTTTGTACTACCATTATATGCCATAGGAAAAATGGCAGGGATCTTTAAAAAGCCACA
TTTCATACTAATAAGAATCCACACTCTAGGGAGACATGACAATTCTAAATGTATACACATCTGAATATATAGCTTCAATATGTTAAACATAAAT
TGGAAAGATGGATATTCCTTCCTTGAAGCCCTACCCTCTCTCAGAAGTTATTTTTCTAACTTTTTCTTTTAAATTATTTTTATATTTTGAAATT
ATAATTATACCATTTTAATTTTTTTAGCACCCCAAACACATATCTATCTTTTTGCAAAGTCATTATTCTTTTTTCATTAATTGTTGCTATAGAC
ATATGTATATACATAAACGCACATAAATACACACAAACACATATATGTATACATATCATACATACATATATATCATACAACCTGCTCAATCTGT
ACAATACCATATATATATACTCAGTATTGAATACCCATTTGGTGTACTCTTCGCTGGGGAAAACTATTTCTCCCACTCTCAATATTCCTTAGTT
GTCTATAATTGCCTTTAGTTTTTTGTTTGGAGTTGAGGCCTCCTGGACTTTTTCACTTTTTCCTAATAAATCCCAGTTCTATTTGTTCAAAAAC
AATTGGAAGGAGAAGAGTAGGAGGAGGAGGAAGAAGAAGAGGAAAAGGAAGAAATAGAGGAAGAGGAAGAAGGGGAAGGAGAAGAGGAAGAAGA
AGGGAAGAAAAGGAAGAAGAGAAAGAAGAGGAAGAAGGAGATGAAGAAGAAGAAAATGAATTTGTGCATATGTGAGTTTTTGGTTCATCTCTGC
TATAGCAGAAAGGACAAGACATACAGACCGGAATGACACACTTAACCATCCTCACCTAGTTAACATGTCTAGAGCACCAACTGCAGAAGACATG
GTGCTTTCTAGGTTCGTGCCTAGCCCTCACCAATGTAGTTATCTAGCAAAGAAGAACAAAGCAAAGTTATTGCAAGAGAAGAACTTCTCATTGA
GTTGGAAATCTTGAACTTTTTTCTTTCATTCATTTTTATTCTTTAATCTTTTTTTTTACAGTTCAGACTTCACCCTCTTCTCAGTCCACCCTCC
AACTGTTCCACATCCCACACTTCCCACACCCCACTCCTCTGTCTCCACAAGGATGTCCCCATGCCCCACCCCACCAGACCTCCCCACTCCCTGG
GGCCCCAAGTCTCTCAAGGGTTAGGTACATCTTCTCTCAACTGAGGCTCCTTGGGTGTAAATATCTGCATCTGACTCTTTCAACTGCTTGTTGG
ACCTTTCAGAGGGCAGTCATGATAGACCCCAATTTGTAAGCACACCATAGCATAAGTAATAGTGTCAGGCCTTGGGCTTGAGCTGAATCTCAAT
TTGGACCCGTCACTGGACCTCCTTTTCCTCGGTCCCTTCTCCAGTTTTGTTCCTGCAGTTCTTTCAGATCGACCCAGGAACAATTCTGGGTCAG
AGTTTGTGACTGTGGGATGGCAACCCCATCCCTCCACTTGATGGCCTGTCTTTTTACTGAAGGTGGACTTTACAAGTTCCTCTCCCCACTGTAG
AGCATTTCATCTAAGGTCCCTCCCTTTGAGTCCTAAGAGTCTCTCACCTCCCAGATCTCTGGTACATTCTGGAGGGTCCTCCCACCTCCTACCT
CCTGAGGTTGCCTGTTTCCATTCTTTGTGCTGGCCTTCAGGGCTTCAGTCCTATTCCCCCCTGCCCCGATCATATTCCTCTCTTCCCCTCCCTG
TCACCTTTCCCACCCAGATCCCATCCCCACCCCCAATTGCTTTCTTCTCTTTCCCAAGTGGGATTGAGGTATCCATGGAAACTGAACAGCTCTC
TATTCAATAACTTGGTCAGGGAAGAAATAAAGAAAGAAATTAAAGACTCCTGGAATTCAATGAAAATGAGGACACAGCATACAAATTTATGGGA
CAATGAAAGCAATGCTAAGAGGAAAATTCATAGCACTAAGTGCCCTGATAAAGAAATTGGAGAGCTCCTACACTAGCAACTTAACAGCACACCT
GAGAGCTCTAGAACAAAAAGAGAAAAAACACACTGAAGAGGGGTAGAAGGCAGGAAATAGTTAAACTCAGGACAGAAATCAACCAACTAGAAAC
AAAGAGAATTATACAAAGAATCAACAAAACTAAAAGCTGGTTCTTTGAGGGGATCAACAAGATAAACCCCTAGCCAAACTAAATAAAGGTCCCA
GAGACAGTATGCAAATTAACAAAACCAGAAATGAAAAGTGAGACGTAAAAACAGAAACTGAGGACATTAAGAAACATCAGATCCCACTACAAAA
ACCTATACTCAACAAAACTGGGAAATCTAGATGAAATGCATTGTTTTCCAAACAGATACCACATACCAAAGTTAAATCGAGAGCAGGTGAACTA
TCTAAACAGGCCCATATCTCCTAAGGAAATAGAAGGAATTAAAAACCTACCAACCAACCAATCAAAAAAAAAAAAAAACCCAAAAAACAAAACA
AACAAACAAACAAACAAAAAAACAGCCCAGGGCCAGATGGCTTTAGTGTAGAATTCCATCAGACCTTCCTTCAAAGAAATCTTGAACTTTTAAA
AGTACTTCCACCTCTGAAACTCATTTGCTCATCTTTTCACAAGAATATTACCCCAGTCCAACTTCCTCATTTTATATTGTTGGGGATAATATCT
CTCCAACTACTTTCCAATGAAAAATGGACTGTGAAGGCATTTCTGATAATGTAACTGTGTAGGGTGGGGGTTTCCATCTCTTCCTGAAACCATG
ATGGTTCCTTTAGCTAACTATCTATCCTTTCCGATGATGTGTCCTTTCATTTACACCCCACAAGGCAAAGTCTCAGCCAAGCACCTGGCTCCTC
GTCCTTCCCCTTCTGAATCACCTTTCTTTGCTCAGCCAAGATCAGTTTCTTGTTTGAAGCCCTCCCATGGAAGTTGCCTGGATCGCCCCTACAG
CAAACTACTCCAAAAATTCGTTTTCCATATTTACCCCCCAAAGTCACAAAAGTAGACATAAGATACAGTATGATATAGTGAAATATTTCTAAGA
AGATGCTGAACCCAGAGAAAATGTATGGCTAATCTAGAGGTGAAATGAGTACTTGACATTTTTACTTCAGATACTGTTTGATTGGCAGAGACAG
CCCAAAACTTAGTTCCAAGGGATTGGGAGGACTAGACCAAATCATTTAGTAGACAGTTTCAGAGTCCAAGTCTCTTTGAATTCTGGGGATTGCT
AGATTGATAAAGAGAAGGCAATCTTTCTGTTTGTGTTATTTCTTGGCCCATTGTGGTATTTTTAATACAAAGTGCTATGATAAAATGCAGAAGT
TCACATCTCTCCATCTCAAGGATTCTACTGTTTCCTTGTTTACAAGCTACGAATCCAAAATTTACCAACCTCATACAGTTCTTGTATACTTTAA
ATAAGATAATGGTCGGGTTACCAGGCTAGGGCCACATTTTATGGCCACTTAACCCCTCCTCTTCTCAAAGTTTTAAAAAGTGTGAGCAGCATAC
AAGAATGCAAAGCCATATTGGAAGTGAACATCATGTGACAGGTGAATGGTGGCCTGGCCACATGATTTTCCCCTCGCCTCCCTATTTGGAGCAT
CTCTGGGTGACTTACATGGGAGAGCATGAATAAGAGCAAAGACCCTCTTCTTCCCTGCCCTCTCCTCTTAATCTAGAAGCACAGATTTTCACTA
GACCCTGTTCTAGTTTTATTTCTGAGGCTTGGATAAAATACCTTGAGGAAATACAACTTATTCTTGAAGGGATTGTTTGGCTTAGAGTTCCACC
TTGTATTCTTTTTGAGGGAAAATCAAGGCAGGAGCTCAGATCACACCCAGAGTCAGGAGCAATGACAAATGAATGCATGCACATTTGCTTGCTT
GTGTTCAGCTTGGTTTCCCTACTTTTATGTAGTTCAGGACCACATGCCTAGGAAACTGCACCACCCACAGTGGGCTGGGTCTTCCCATATCAAC
TTAATTAAGACAATACCCCATAGAGATGCCCACCAACCAACTCAATGTGGACAGCCCCTCATTAAGGTGCTTGCCTCAGATGATTCTAGATTGT
ATCCAATTGACAATTAAAGTTTACCATTACAGACCCTAATCCATTCATAAAAGATAAGAACATCTTTCTTATGCCTGGGGATATAGTACAGCAG
AAGAGCACTTGCCTAACATGTACAGTGCCCTGGGATTAATCCCTGGTGATGGAATTAATCTCTGGGGAAACCCTGATTCTCTGTGTCTCTGAAT
ATAACTAGTCAAATTAATAAGTTATTACATGGAAGCAGAACAGAGAAAGACAGAGAGGCAAATCACATGGGAAAGGCTCTGAGCATGTCTTCCT
CCTCACTGTTCTACATTAGGTAAAAAGGTCATGCCGCAAAGCTTACAGTTAAGATTCTGTGAGTGCTATAGGTATTTAAGGCATTAAATACATA
GGTATTTAAGGTTTTTCACATAACTGTTATCCACCTGGGTAAAGTCATGCAGGGTCTATAATCCCAGCACTTAGGATACGGACACCAGAGGGCA
GCAAATGTAAATTCATCCTGGAATACGTAATGAGGTCAAGGCTAGCCTAGGCTACATTGAGAGAGCCTGTCTCAAAAACAAAATCTAAAAAATA
TAGTTGCTATTAGTTTAATATGAAATGGTCTTTCCAATGAGTGTTATGTTCTTATATATTCCAGCAGTTTAAGAAACTACCTTATCACACCTGT
TATGATTGCCTTTCACCACTGTGATACTGCACTTGACATAAGTAAGTCAGGGGAGGGAGAATTTATTTTAGCTCACAGTTTCAGAGGATCCAGT
CCATCATGATCAGAGGGATACAGCAAAAGAGAACTCCCATCATGGCTTGTTTTCTCTTTAGCTTGTTTGGGTTCCCAGCCTGTGGGATGGTGCT
GTCTATGTTCAAGGCAGACTTCCCACCCACTAGATAACTGAGCTTCTTGTCCAATCAAATTGACAAGATTAACCATCTCAACAACTGTGGTTTT
AACTTCAAGATTGCTTTTACTTCAATGCCTGTACTACTTATTTAAGGAAATATTTCCCAGTTAATGAAAGGCAGGACTCGCTCCCCCTTCCATC
ATCAAAACCCCATAGTTGATTGTCAACACTTCCAGCTATATACATGTGTAGCGGTATTCGTTAATTTTTAGGTGTGGCCTAAGACTGTTGGACA
GTCTTTGTGCCTTCCTGACTCTCCAAATATCTCCTTTTGGGTTTAGATTTCCGTGTTCTAGAAAGTAGAAAATCACACATGAATAGTTCATTTA
GTGTGCAGTCACAGAACATATCTGTACTGAGAAATAAGAAACACACCCCAGCTCTTTTTTTTTTTACAGCTCATACATGAGTCCTTATTTTCCT
CCTCCTAATCACAAAGATAATGGAGGGAAAGGAAGAACTTTTGAGTAAACTTTCTACTGATGCTGTCTGAAAAGTGATCTATTGGGGTAATCTA
GCCCTAACCATTCCCTCAGCTTTCACCATCCTTCAATTATGAGACTGTTTAGAATACAAGAGACCACAGAATATGGCATCCTAGCACAGTGCTA
GGATGTAAATTTGTAGCCTTCCTACTTTCAAACATTGGGTCTATCCATCTTTTTTCTTCTGACTTTGGATACCACTGTCAACCTGTCTTCAGTC
AACCTCAACAAAAATTATGTGTATCAAACAAAACATCTTTGTTGTACTGTATGAATAAAAACTATTCAAACTATACAAGCTAATGAACCTGAGA
CATAGGAAATATTTTTGAGTTTAGAAAAATGTATCACCTAAGATCAATAGTAATCACGTGTTTCTTCTTCCATTGAGTACAACTGTACAGCAAT
CCAAAACAACATTTCTTTTTCTTCTGTTGTCCAGCTTCTTACATGAGGATAGACACTAGGATCCTGGAGGTTCCAGGTACAGGGGAGGTGCAGC
TTACCTGCCAGGCTAGAGGTTATCCCCTAGCAGAAGTGTCCTGGCAAAATGTCAGTGTTCCTGCCAACACCAGCCACATCAGGACCCCCGAAGG
CCTCTACCAGGTCACCAGTGTTCTGCGCCTCAAGCCTCAGCCTAGCAGAAACTTCAGCTGCATGTTCTGGAATGCTCACATGAAGGAGCTGACT
TCAGCCATCATTGACCCTCTGAGTAAGTGTGGCCCCCATTTCCTAGTCCTATCAATTAGAGTTCAGACGGGAAAAATCTTAAGTGTAACTTGTA
TTCACAAGAGATAATTGTCATTCAGAAGCTTACTTCTGAATAAGCTTTCTAGATCATTTTGAACTCTGGCTGACTGGTTCAATTCAGCTCTTCT
GGATCAAAATTCCTCTCCCAGCTGACTGGTTCAACCTGAGTTCTCTTGGCTTCTGACTGAATTGCCCACTTGGCCTCAAACTAACCGGCAATAT
GTTTAATCTTCTGTTTCCTTCTCAGTCTCTGGATCGTTCTGTCTTCACCTGTCTCTGTAAAACTGCCTCCTCTCTCTCCTCTCTCTGTGTCTCT
CTGTTGCTGTCATCTCTGTCTCTGTCTGTCTCTCTCTCCCTCCACTGTTCTCTCTTAAGCCACCTCTCTTCCCTGTCCTCATGAGAGTTGGGCA
TATCCTATCTCTGACTCATTCTGTCAAGTCTTTCTCAGATTTGTCACTTTGTCTGCCCCTCGTTTTCATACATGGCTGCTTCCTTCTACAAACT
AACCAAATCTTCAAGGTTTGGAATTAAAGGTGTGTACTAAGGGTGTGTCTGTATTCCAGATATAGAGATTAAAGGTGTGTAATTCCAGCCAGAT
TACATAGATCTAGGTTTTTGGATGTGATCCCTTGCCAAAGCAGTCATGTTGCTGGATTAAAATTCCTCTACAAGAAACAGATGGTGCGCTCATA
TGGCTTGTTCAAAAAGGGGTTCTAAGAGACTCTTCATGAAGTCGAGATAGAATATCGAAGAACTACAAACATAGAATGGGGCAAAGATCTCTAC
TGTTGAGAAAGAGTTACCAGTGTTGGAGAGGCTACCAGGAGAAGACAGCTGTGAAGCAGCTCCTCTGGGAGTGCAGCTTCGAGGAGGGAAGTAT
GTGACATACCACAACCACTCTGCGGGTCCCCCAGTGTCCTACTGGTGCCTCCCATTGGCTGGTTTGTGTGTGTGTGTGTGTGTGTGTGTGTGTG
TGTGTGTGTGTGTGTGTGTGTGTCCCCGTCCCTTCTTCACCATGTGTTCCCTGAACTTGATCCAAATGTAGAGAATCCACAAAAGAAGATTGAT
CAGCATGGGTTTAAGCCAATAGAAAGTCTGTATTAGCCAGCTGTCCATCATACTGTGTGTTTGAGATTATGGTGTAGTTAACCTTTTTCAGTTA
ACAAGAACAGTTACCAGAAGCAGAACTACAGAAGCCAGAAATAAAGCAAGGTTAGTTAGTACATTTAGAGAATTTCCTAGAACTATGAACTTGG
ATGGATAAGGTCTTTGTTTTTATTTGTCAGATGGGGCTGTCCTTGGGCTGAGTTTTATGACTTGAATGGCACATCCATCATGGAGTCAGTTGTG
CTAAGGTCTGAAGGCATAGTAACCTCTGGAGCTCTATTACATTCCCCACTGGCCCTAGAGAGTGAGTTAAACCATAGGCTCATTCTATTTTAGT
TCAAGGTATAAGGGCCCGTGGTTGATCAAATCAGGAGAATGACATGTGCAGTGAGCACTAATAGCAGATAGTTAGCCAAGGGGACTACGAGAAC
AGACACTGGCATCAATTATCTGACTTAGGGCTCTGTCTCTTTTCCTTCCTTCCTTCCTTCCTTCCTTCCTTCCTTCCTTCCTTCCTTTCCTTTG
TTCTTTCTTTCTTTCTTTCTTTTTATGTTTTCTTCAACCATAGCAAGACTTTCTCTAATTACTCTTGATCAATTAGCATAGAAACAACAGGCTC
TTCCCAGGGCTAGACATAGTTTTCTTCATCTCGTGAGAAGTCTAAACCTCTCCAATTTTATAGGATACTTTCTGCAGGGGAATCTAACTACTCT
CCACCAAATAGTCAGGGGTTTGGTGATATTGCTGTCATCCAGATGAATCAGCCCATAAGGCAGAATAGCAGGCATCAGAATAAGGTAAGCTATT
ATGTTAAAGGTTTTGTATATTAGGCAAGTTTCAGGTCCTTGTAAGTCCTGTAATATTAATTTGTGTTGATCCTAGTCACAATGAATGTTGTCAG
TCAGTAGACTGACAGTCTAGTTAGTTCCTATCCCTCTGTAGTATGAGGGCTGGGCTGTCTAAACATAACCAGCCATCCTGGCCAACTTCTGGCT
GGGAGTGATGAACTCCCGGGGGCCCTTCCTGTGGCATAAAGTCCGATTCCCCAGGTTTTCCCTGTTTCCCAAAGGCCAGAACTTTTTTTTAAAG
AAGAATTTTAGGAAGCCTCAGTTTCACAACCCTAATTTTTACAATGGAAACTTTTTTGTTTCCTGGGGCCCAGTGAAACCTTAATTTTAATAGG
TCACAGTGCAGGAGACAGTCCATTTGGCAACAGTGTCTGTGGCTTCTGCTTTCTCGACCTGGATGTACTGAATCCATGGTGTAATGCCAGTTGT
CTTCACAGCCATAGGAGTAGAAAGAATCACTGTGTAGGGTCCTGTCCAAGTTGTTTTCAGTGCCCTTTAGGGCAGCCACTTGACTTGAACAAAT
AGAAACTGGTGGTGAACTTGGAGGTTGGCTGGGTCTCTCAGCTAGTCCAAGACTGAGGGTCTATAGTGACTGGACAGAGGAATAGTCAGAGAGT
TCTGTCAACTGCTGGTCTCTGAGCTCGAGAAACAGTGGGGGAGGTCTTCCAAATATAATCTCATAGGGAGTAAGTCCCTCCCCATATGGGGTGA
AACAGGTCTATGCAAAGCAAAGGAAGGAGGCCTATCCACCCCTCTCATTGGACACTAATGAGAGTTTTGTTAGGGTTTCTTTTTATGTCCTGGT
TATTCTTTTGACCTGGCCACAACTCTGAGGTCTGTATGCACAGTGAAGTTTTCAGTCAGTGCCTAAAGCTTTAGCTATTACTTAATAGGTTTTG
GGCTGTAAGCTACGTTATTGTCAGACCCCATTGGGAGGGGGAGGCTAAATCAGGGGACGTGGAGGCCAAAAGGACCTGGAGGAGTTTTTAAAAC
TACTATCTGAGCAGTACTGGAAAAACTGGCAAGTACTGGGTGGTGGTGGTGGTTGTTGTTGTTGTTTTGATCCTCCTCTGGCAGGCTAAATGTT
GGCAAAGTCAGTTTTACTGTTTTGTGATGTGTTGGCCTCTTATTTGGACCCCTTCCTGGGAAAGGGCTCTCTGTTTTGCATTTTCTTGAGCACA
AACCTGGCATCTGGTTGAAGCCTCCACACTAGGCTGTCCAGTCTAGGTATAACATACCTTGTTTTGAGCAGCTCAGAAGTTTTGTGGACCCCAA
ATGAGTGGCCTGGTAAAGGTCAGTTGCCAGAATCCTGCCTGTTGTTTCTGAGAGAACAGTCTTTCTTTCTGGAGTCCTCCGTCAACCTGTGTGT
TTAAGCTGTCCATTTCTCCCATGGATTCTCAGCCAGTACCAGGTCAGGGTACACCATCAGAATACCCATAGACCCATTGGTCTTAGGGACATTT
GTCTGGTAGCCAGGTCAGCAGGCAGGCAGGAGAACAGCAGTTCATGGAGGAAGCCAAATAGCCCCTAGGAGGACAAAGATTTTCTAATGTTCTT
TTCCCCAACACCAAGAAACCCTCTTTCTCTACAGATCATGTCATGGACATGTCTGCTAATAAACGTATAGCAACTGCTGGTGTATATGGTAGTG
GACTTTCCCCATCTGAGAGTCTGGGTCAGATCTATTCTGCTCTCTTGACTGAGGCACCAAATTTGGACCCAAATCCTTTCCGTTAAAGTAACTA
CAGCAGCTCCCACATACCTGTTGTCATCTTTCATCTCCTGACAGTCACGGATGAGCACTTCTGGGTTGTCAGCAGGAAAGAAGGCAGCAGGATA
GGCAGTGGTGGGGTTTTAAAACTGGACTCGAGGGTGGGCCAGGAGTTGGGCCTGGAACTGGGTCACACAGGCATTAGACAGAATTCTAGTGCTC
CTTAAAGGAGGGCCTCAACTGTGCCATGGGGTGTTGTGAGCATAACATCTTGACCCAGAGTTAACATCATTATGATCATTATTAATTAGTCTAG
TAGTAGCTGCTATATCTCTTAAATGGGTGGGCCATCCTGTGGCTACAGGGTCCAGTCGTTTAGACAGGTATGTCACAGGGCATTTCCGTGGCCC
CAAAGTCTGGTTTAAAAACTCCCTTCTGTCTTGTTTCATCAACAAACACATGAGAAAGTTTCAAGACATCTGGAAAGACTAGGGCTTGAGTGGA
AGTCAGAGCTGTTTTTAAAGCCTCAAATGCCTTTTGTTTAATCTGAGTCCAGATCAGAGGACCAAAGCCTGCTCTGTGCTGGTGTAGAGGCCTT
GCTATTTCCACAAACACTCGTATCCAGAAGCAGCGATGTCCTGCTAGACTGTCTCTCAGACCTCAGACTCTCAAACCTGTCCGTTAGTCTTTGG
AATTGGGATCTGACAGATGGCTGCACTCCTACTCACACAGGCTCTTTTGACTCCCTTCAGCTGGCGGCCAAGACAAGTAGCCTCCAGTGTGTTT
TCATAGTTCGCGTGAATCTTCTTTGTATCCAGATCATAGTGAACGTCATGTTTGGGCCATGACAAGTGCTGCCATGACGAATTAGAAACATAAT
CAGTCATAAAGAACCTCAGCTCCCTCATCTCAGCTCCTAGTCTACGTAGACAGACCAGATGTGACACCCTGCCTTGTGACCCCAAACAATAGCA
GCAGGATAAACACAGTCACATCCTCCCAAGTGGTCCTACCTCTTAAAACCATTGTCTAGCTGATTCTTCTCATTCTTATCAACCGAGCTACTCA
CCCACTGCTGGCTGCTGACTGCTGTGAACCACCTCAGAAATCAGTGAATAAAACAAATGCAGAAGTGAGGTGCGAGGATGCCTGCTACTCTAGA
ACAGAAGGAAGGCAGAGGGTGCCGGACACTCTAGAACAGAAGGAAGGCAGAGGGTGCCGGACACTCTAGAACAGAAGGAAGGCAGAGGGTGCCG
GACACTCTAGAACAGAAGGAAGGCAGAGGGTGCCGGACACTCTAGAACAGAAGGAAGGCAGAGGGTGCCAGACACTCTAGAACAGAAGGAAGGC
AGAGGGTGCCGGACACTCTAGAACAGAAGGAAGGCAGAGGATGCCTGCTACTCTAGAACAGAAGGAAGGCAGAGGATGCCGGACACTCTAGAAC
AGAAGGAAGGCAGAGGGTGCCGGACACTCTAGAACAGAAGGAAGGCAGAGGGTGCCGGACACTCTAGAACAGAAGGAAGGCAGAGGGTGCCGGA
CACTCTAGAACAGAAGGAAGGCAGAGGGTGCCGGACACTCTAGAACAGAAGGAAGGCAGAGGGTGCCAGACACTCTAGAACAGAAGGAAGGCAG
AGGGTGCCAGACACTCTAGAACAGAAGGTGGAGGATGTCTGACACTCTAGAACAGAAGACTGAGTGCACTAAGTTTTGGTCAGCAGGTAGGGAG
TCATCAGGTCATTTTCCTTTAGAAACAGGGCTGTCCTCAGCCAGTGGCTACATTTTCACTGGCATATGTAACTGGGCTAGGTCATGGGATTAAC
CGTAAAATACCTGGTAGAGCTGGCAGAAAAGTGAAGCCCTCTTGGGACCGCTCAGGTAAGGAGCTAGAAAAGAACGTGGACTCTGAAGCCAGAC
TTCAAACTTTCAAATGCTATCTCTGCCGCCTCTTGGCTTTAGAACTTGACTACCCTACAGAGCCTTCTGGGAACTCAGTCTCACAGGTATACAG
TTACAGTAAATGATGATACACATGCTTAAAAAGTTACTGTGTAGGTAAATGAAATATGATACTAACAAGGAGTAGGGAGGCCACTTTGTCACCT
CCAGCAAGACTCCTGGCTTCTTTCTGAGTGCATAGGGCTATGGTAAGAATAGGATGGATATTTTTTTCCATCACATATCTTATCATTGTTAAAG
GGAATGCAGTTATCTTAAAATAATAGAAAAGAATATGTAGGTCACCCATGCTGCCATAATGAAGATTCTCAAACATGCAATGACTCAACACAAC
TGTGTATTTCTCTTTACATAATACTTAGAGAGAGAGAAAACGCTTCAGGCAGCTAAACAACTACCTTCCGTAAGTCATTCAATGACCCAGGTTA
TTTCCATATTGTTACTCTGTCATTCTCCGGGATATCGTCCCTGTTAAATTGTCAAATCAGGGTCATTGCCAAATCTATATCCCTGCCTTCAGAG
AGGAAAGAGTAGTAGAAGAGAACTTTGATGTTCTAAGTCAAGATCTGGAGAATTCACTCTTAGCCGGAATTTAGTTTATACTACAGGCTATGTT
TAAGTGCTGATGAGGTCTAGAAAAGCAGCCCCTCAGAGAGCTCTATGTCTAACTATTAACCTAGCACTGGGAAGGAAGGAGCAGGTAAGATTAC
CAGCAGGTTGCTACATGGTAGCTACTAGAGGGAGTAGGTAAGATTAATAACCAGCAGGTTGCTACAAGGTAACTAACATAGACTAGTCAATTCA
GCCTAGGAACTGAGACTTAAGTCTCTCCTGCAGTGGTGCTGGGCTTCATACCAGACTGAGTGGTGGCTTAGGAGCCAACTCTATCCTCCAAATG
CATTCTAAGAAAACTAAAGTCTTCAAGGGTAAGCAGAAATTCCATACATGGAAGGATCTATGAGCAGACCTGGACAGAACAAGGGTTCCCAAAG
ACGCTGAGGCAGGGGCCTGGCTCACAGCACTACCTTGCAACATGCATGTAGTAGAAAAAAAGCAGTGTGATCAGTTAGGGTTTGCAACACACCT
CTACCTGTTGCATGTAATTTTAAAAATATGCTATCTCCTTGGCAGATCTGCTGCGCCATCCCCACTCTCGGAAAAACCCATTCTTCCCAGCTTA
GCTCCGCCAATGGCCAGCTGGTGCCTTTCCCAGGAGCTGCGATTATGCTCCCAGCCCTTCCCAGCTAGCCACCAGCTACTGCCTCTCTCTATCT
CTGCCCACCTCTCTGAGACTTAGACAGCTTTATTAACTTAGTACTTGTCAGATAGCATAATTGCTGGGAGCAGAATCTCATTGTTCCCAAGCAT
GGCCACAAACTCCTTTCTCTTTCCTCCTCCTCCTCCTCCTCCTCCTCCTCCTCCTCCTTGGACCCGGAAGTTCTACCTTTACCCTTCACCCAGC
AATTAGCTTCCACCTTCTTTATTGACACAATCAAGAACCAATTCAGCTCCTCTCCCTACATCTACCAAACTGGCCCTTATCTTTTTGGTTCTAG
CGTTCACATGCTCTTGGATAAAGATGATAGCTCTGGGGCTTGAGTTTTGGATCAAGAAGATCTATATCTAAAACCCTGGCTGTTTTACTTCCTT
GGAAACAATTACCATAGACAGATTAGCATCCCTGAGGCTTCCTTGGCTCTAAAGTGACTCTGGATTATATATATATATCCTTCCTTAGCCAGAT
TTTCCCATGGTCCCATACTCTGTAGAGGGAAGGTCAAGAGCAGAGCAGCCAGCAGAAATAGGCATAGGGTAACTTGTGTGCCTATTCTTTGCCA
TGTTTTCAACATCTCTGTAGCCCTCTTTTCTCTCCATCTGGCCAGCCTGAAGGTACCAGTGCTCTGCAAAGCTGTTCCCCAGCCTTTAAATTAA
ATCGCCTCTTCACTAAATGGATGTGTCAGTGGCAGCACCTTGGTTAGTTCAGACAAGATAAGGGGTAACTTGTATCTGAATGGCTGGATTTAAC
ATGTCAGCCCATTGGGGATATTCTGCTCTTTCAAAAGGACCTTGCTGGTGTTTAAAATTGAATAACTAACAGTGCAGAGTCTAGGCTCTTGAGT
TCTTATCAGTGAGGGAGAAAATACATTTTTGAGCTGAGGGACTTTTTCTACTTGAATGAGAAAATCCAGATGGTGAAGAAAAGAGAGATGGAGG
TAGAGATAGCGACAGAGACAGGGTGGGAGGGAGGAAAAAGAAAGATGACGAATAGGTGGGTGGGTGGAACAACAAAGCGACAAAAGGACTGCAT
CATAGCTGAATCACCTGCTGTCTAACTTTGGGGACATTTCTCAGCCTTTCTTCTTTCATGGGTGAATGTGGAGATCAAAGAAGAGGCTGCCTCA
CTGGGTGGAGGTGAAAGGAAAAGAACCTCTACTTGAGAGAGATGTGGAATGAAGGCTTAACTTCATTTCCACGTGGTAGAAACCACACAGGAAG
CCACCTCAGTCTGTCTCGGGGCCACCTTGTGATGAGATTCCGGTTCTCTCATTCACCCAGGTCGGATGGAACCCAAAGTCCCCAGAACGTGGCC
ACTTCATGTTTTCATCCCGGCCTGCACCATCGCTTTGATCTTCCTGGCCATAGTGATAATCCAGAGAAAGAGGATCTAGGGGAAGCTGTATTAC
GGAAGAAGGTGAGTGACTTACAAGCCATGTAAGCCTGTGGCCTGCGGGTCTTCAGAGAAGCACTTCAAGCCTAGAAGGGTTTTGAGATTACCCT
GGGGACATTCTACTCCAAGAAGAACATGGTCCAGTCTTCAGTGAGCTGAACCCACGCTGAAGTTTTGTGTTGTTTTATTTTGGTTTTTTTGTTT
TTTAAGGGGAAGTTTGTTCCAGGGCTGAAGGAGTTTGGCTTGCCCTCCAAGCTTCATGGTAGCCAAGTTATAGAATCAGCCAGATTCCCATCAA
TGATAAACAATTTTTTAAAAAAATGTGATATGTGTGCATAACAGAGCACCTTGTAGCCTTAAAGAAAAAATTAAATACTGTCATTTTTCAGGGG
AAGAAATGAAGCTAGAGGTCATCGTTTTAAGCCCTACTCAGAAGGTGCAGACAGAGATTATATTTCTTTTGATATATAGATTCTAGACTTAAAA
AAAAAATAAATACAAGGACAAGGTACCCGCACAGCGGAAAGGAGATACAATGATGGAAGTGTATATGTGTGTGAAAAGCCATGATGATTAGGCT
GGAGAAATGGCTCCTGAGTTAAGAGAGTCATCTTACAGAGGACTTGAGTTCAATTCCCAGCATCCATATCAGGAAGTTCCTGATCACCTGTAAC
TCCAGCTCCAGGGGATCTAAGGTCCTCTTCTAGCCTCCCAGGGAGCTAAGTTAGTCCACCAGAGGGCCCTATCTGAGCCAATGCTGAGGGCCTA
GTGGGTAAGGGGTTGAATGTACACAACTGTGGCCGACAGGAAGTCCCACCTGCCTCTTCAGCCATATGTGTCTTGCACAGAGACCAATGGCAAG
TAGCATGTGAGGATGCCATAACTAATTCACTGCCACTGTGGTGTAGAGCTCTTGAGTCCTTGCTAGAGCAGAGTCCCATGCAGAGCCCATTAAA
CTTTCATGGCTGATGAGGAAAGAGGGGCTTCCTTCCTTCTCGTCAGCTGCACCATTTGCCCCTTCTACACTAGACCCTCATAAGGAACCTGAGT
CAAGAGCTTTAACTTCTCTCTCTTCTACAGTTCTTTGGGCCATTGTTGTCCTGAGACTACAGTAAAGATAGAGAAGTCTCCATCCCTTTCTTTC
TAGAAGCCTCCTCGGCTCCTGTACAGATTTATTCATCAGATAATCACAACTTATGATGCCTGCTGAGTACTGGGAGGGCAGCCATTGCCAGTCC
ATAAAAGTGTCTTATTAACTATTCCTTTCATAGAGCCATTCAATCCTTTGTTGAAGGATCCAGTGTGAAACAATATGTTCCAGAGAAGTTGGCA
AATTCTAGTTCTGTTTTATCAGCTAGGCTTAAGTATATGGGAACTATGTTTCTCTCATCAGTAGATATATGTTCAATATCTAAAATGCTATGTG
GTATATATATATAAGATAATGCCCAATAAAATTGTAAGTTTTTTCTTTTTTCATCCCTGACAATTTCATACATGCATATATATGAACCTATCAT
GAACTTTAGACATTCCATCCCCCTTTCTCTACTCTTACCTCCCCTTCCTTTCCTACTGACGTTTTCTTTCCAACAAGTCTCCTCTCTACTTTCT
CGTTTTCTTTGTGTGGGTGGAGCCCCCTGAGCTTAATTGAAATTGTTTGCGGGAGCATGAGGCATGAGCGTAGGTTACTTATTAAAGGTCAACT
GACCGGTAGTTACACCACTGAAGAAAATGGCACCCCTTTCCATAGCAACCATTACCTCCCAATAGTGCCTCAGAAGGGGGCATCGTGGATTCCT
TCTCATCCATAATGAAATGTAGGCAGGACCAATCTTGTGCATATTTGAGCAAATATACAAGCTATGCTGGGTTCCCGAGTGCACCAGCTATGTC
TGACGTTTTGTTGATCCTCCCCCCACCCTCTGTCTCTCATATTCCTTCCTCCCCCTCTTCCCATGATGTTCCCTGAGTGTTGGAGGGAGTGAAA
TAGATATCTCATCTAAGGCAGTGCTTACTCTTGGATTTTGGTCTGTTATGGGTTTCTGCAATGCTCATTAAACTTGTAGTCTACTGTCTGTAGG
TGAGCATGACAGACCTGCTTTGGCCATTTGAGTAGCCATATTTAAAGCATTCACATGCTGTAGTTACAGGCTACAATATTGGATACTGCAGACA
GCCAACATCACTGCACATCACTGCCCAGCACTGTGATAAATGTCCCCCTCAACTCCTCTCCAAATTACAAGCTTTGCCCAGGACAGAAAGCAGT
GCCTTTATTTTGTCCATGAAGGAACTGAGGTTCAGAAAATCCGTGTGACTTCCCAAAGTCACACAACATAGACAGTTGAACTCTAAAATGTGTG
ACTCTTTTTATGGCAAAAGAGCTTAATTGAAACCAGCAATCTGAATAGTATTATCCATACAAGGGAAGAGGAAGTCGTAACCCAAAGAATTAAA
TGAGTTCTCATTTACATGCTGGGCCAATCACGAAGCCAAGTCAGAGTCCCACAGAAGTTACCTGTACAGAGTCTAGATCTGTAGAGCTTATTAA
ACTACAGGTGGCTTAAATGAATGAGGATTAAAAAAAACTTGGCACATGCAATGAGCAATAATCAATTCAAACATACATATATAACAAATGCAAG
GTGCTTATGGCAGTTCTCATTCATGTTATCACATGACTTCATTAAAGCATGGTCTGGACATACAACACAGTCACTTTACACTGTGCATGCTCTT
CTATAACTGAATGTCAAGGATGCTGCAGAAACACCTTGGGTCCAAGTTGAGACTATTCTGTGTTATGGCTTGCAGTGTTTTTATTGTACATACA
AAGTCTTATTATCTCATTGAAATAGAATAACTTAATTCTAAAAGTATATTTCAAGACAAGGTCTTATGTCATACAAATAATATAAGACAAAGTC
TCATTACATAGCTCTAGCTGTCTTGGAACTCACTCTGTAAACCTGGCCAGTCTGGATCTCACAATGATCTGCCTACCCCTGCCTCAGAAGTGCC
AGGATTAAAGGTGTGAGCCACTACATGCCTATATAAAAACACTTTTAATATTTTTCTACCACTTTTCCTCAATATGTGTCAAATTCATATAACT
TTATATTTAGTTGCATTAGAAATTTTATTTCTAAAGCTGATTTAATTGCTAGCCTGAGTTTCATTAAAAAGTTATCAATGAGTTTTGGGTTGTT
ATTAGGACAGAATTTCCAAAAATTTCTTAAGTGCCCCTAAATATACTTCTGCTAATTTGTGTTGTATATTTATGTGAGAGGTGTTCTCTGCCAT
TCACCATAAAACTATTAATTTTAAATACAACTAACTCACTAGTATAAAATTTTTCTTTTGTCTTTAATAAATGGTAGAATTATATATGCACAAA
GAATTGCTTTTAAATAAGTTAATTATTTCATACCAGTAAATGCTTAGTTTCTGTAGCTATACACTGTCACACATACATCCTCCCTCACATACAG
TTTCACACACACACAGTCACACACTATCACATATACAAATACACTCACATACACCTTGTCACACACACCCTGTCTTTCTCTCACACACATACCC
TGTCACACACATACACACACACACACACACACACACTCATACACACCCTGTCACAACACATACCCTATTACACACATACATACCCTGTCTCACA
CACGCACACACATACACACTCACATATACCCTGTCACACACACATTCTCCCCCACATACAGTCACATACACACACAGACACAAATTCTTATATA
CACACCTACACCACACATACACACATCCCCCTCCACATACACATAGAATAAAATAAAATATGAATAAAACTTGGGACTACTATGTTCTTGTGTT
TTCCCATTTCATACAGACCTCTTAGTTACTATGTAGCCCCAGCTAATCTCAAACTAGAAATTCTCCTGCCTCAGCCTCCCGAGCCTGGTACAGG
TATGTATCCTTGGCTTCCTATTGCAGTTTTTAAGAACTCCTTTACAGTGTATATTATGAAAGTATCAACAACACTTTGAAAATAAAAAGCACAG
ATGTAATACACACACACACACATACACAAACAAATCTGGTTCTTGCCAAGATATGGGAAATATGGTTCTTAAGATGTGTGGAGACTTGTGTGTG
CACAAGTGTGTGTATGTGTGCATGTACATGTGTGTGTACATGTCTGTGTGCATGTACATCTATGTTCATATGTGACCAAGGTACATCAGTATGC
TGTGTGTAGAATACAGATATCAACCTTGGGTGCCATTCCTCAGGCACCATATGCCGTGTTTCCTCCACACAAAGTCTTCCATTATGACGTGAGG
CTGTTCACTGAGCCCCAGGGATACATTCACCTCCCAGGCATGTGCTACTGCACCCAGCTCTTTACACAGATGCTGGGGATCAACTCGGCTTCTC
ACCCGTGTGCAGCAGCCTCTTTATTGAGTTATTTTCCCAGTCTTCTCCCACATTTTTTATTAGATACAACAATACAGCACACTTCAATAACAGA
AAAAACGGCCATAATTCTTATAAAAGTCTCTAAGGTTTTAGATTCGAATATTAATCCTCGTTTGATCATGAGCTGGTAACTAGCAGTTTTTCAG
CCAACATCATCCAGCCTAGGGACCAGCACCAGGTTGGTGCTGCCTTAGAAGACCGAGTCGGGACTTTGCCAGACTCTAAACCAAAAGTCAAAGG
GTTATGTTCTGGCTGCCTATGGCTGGTGGAGAATATTAACCCTGACCTCTGGCAGTAAGGGGGCTGCCACTGAAAACCGAGACATTCCAGAGC
AGCTCCTCTGAGTTGAGAATTGGGTTCCTTTCAAATACCAATCTTCTTGAAGGAGACTACCCTGGCTATGGCACTGAATTGATCCTTGACTCTG
AAAGCCCAAGCTAATGTCAGGATGGTCATGATGGAACTGGTAAAAAGCCAGATAGGCAGGAGCAGCGCAGGACAGTGGAGAGCCAGGTCCTAGA
AGAGAATCTGATAGGAAATAGCAAGTGCTATTTAAGGAAGAAATAAAACTAGAAAAACCTGGTAACAGCAACTCATCGTGCAAACGTATGCTTT
TGTTCTCTTAGCAACTCTGACATCAGTCTTTTACACCTTAGTTAGTGTAAATTGAGCAATCAAGCCTTTTGTTCTATTCCTGGACAGCCAGGTA
TAAAGCGTCAGTCAAACAGGCCATTCAAAAAAGCCACGTGGGAACCTATTTTACAAGTATATATATTCTTCACCTAACCTATGTATTCATAGAA
TCTATATATTCATATTTTAAAAGTTTAATTTAGAGTTACTCTACATAGAGGATAATAATAATGTTTCTCCCAGAACCCATAACAAAAACAAAAA
GCCAAGTAAAGGTGTGGGTTGATGCCCCTCCTGTTGTTTGTCATGGGTATCCTAGAGACTCCCAAAACAATACAAACTATTGCCATTGCTCTTG
ATTGCCCCCCAGAACCTGATGGTGAGTCCTTATTCTGGTAGATACAGGACACAGAAATATTAAGTTGGCCCTACCCAGGAAGCTTCTTCCCTGC
TGGCCACCTTTCATAGTATCTCAGGAACAATGTGAGCTGCTAGGCGTGGGGTGAGCCATCAACACCTTACCCAGCGGAGAGTTCTGTGAGCCAC
AATAATGCCTGTCATGGCAATATATACCCATGGGTACAGTAGTAGCAATAATATCACAGTTATGAACAATCTGATATTCATCAATATTCTGATT
GAATCTAAGGCCCACTCTATAGGAGGAGCTGCCTGGTAGTTGGTATTGTATCTGTGACCAAGAATCCATGGTTGGCGAGCTCAGAGGTCCCGGT
GGTGAGGCTACTACAATGATGTTGCTAAAGGGACAACAGTACCAATCATTTCTCTAAATTTCTATCTCTATCTTCATAGATTTCTGCAGCTCCC
AGACCCCATCATGAAGTTTCTTTGTGCAGTGGACAGTGGTTAGCAAAGAAACTCATAATGGCTCAAAGTTCAGAGAGTAATTGTCTATGGTGAA
CTCAGCCAAAATAGGACATCAATATCGCACCCTTACCTCTTTAGGTTTACAGACTGTCTTAGTTAGGCTTTCACTTTCTGCAACAAAACACCAA
AGGGTTTATTTGGTTTATATTTCCACATTGCTATTCTTCACCAAAGGAAGTCAGGACAGGAACTCAGGCAGGGCAGGACCTTGGAGGCAGGAGC
TGATGCATGCTTGCTCTACATGCTTTCTTATGGAACACAGGACCACCACCCCAGGGGTGCCACTGCCTATAGTGGACTGGGTTCTTCCCCATGG
ATCACTAAATAAGAAAATATCCTACATCTGGATCATGATGTTGTAGAAAATATTTAATCTCAATCCAGGTTTTCTACTCCACCTTTGACCATTT
AGTTCCCAGATAAAAGATACTCATAACCTTTATATTTACAATAAGCCTTAATCAGCACAAGAGCTGGGCAGATATTTATTCTCCATGCTATTAT
GTCTATTTCCTAGCCAATAATCCCATTAAATAATCTGCCATGTTTTGTCTGGGCTGCTCTTAACTCCAGTTAGCCAACCCATGTGGCCATTATT
TCAAGATTCTTACCCCACCTCAGCTTCTCCTCTCTCCAACTTCTTCTCTTCCCACCTCGTGGTTCTCCTCTGACCCAAGCCTGGGAACCCTACA
TCCCGCCTATGCCTCTTCTGCTCAGCTATTGGCTGTCAGCATCTTTATTCACCAATTAGGGATAACTTGGGAGTCAAGGTCACATAGCATTATG
TGGGTCTATATGTGGTCTCTGGGAGTAACCAGTATTTCGCATAGCAAAAGACCAAACCTTAACATTATGGAGGCATTTCCTCATTTGAGGCTCC
TTCTTCTGCAATGTCTCTAGCTTGTGTCAAGTTGACACAAAGCCAGCCAGGACAGGGGTCATCAAGGAGTAAGAAACACATTATTAGAGGCGGA
AGTTGGGAAGGACAGATCTAGAATAGTGTTTTCCGGACATTAATGGGACCACTACACTCATAAGTTTACAGCAGCTATAGAGACCTGTATAAGA
CCTGAACTAGAGGATGCCTTCACAACATTTCAGCACAGATCAGGGAGGGGCTCGTGAACCCCATACTCAGCTGAGAAGTTATTGACAGGTGATG
TCATCTGAGGGAGGGAGGATCAGTGAAGGGTATGGGCTCTGGTATGTTGGCCATGCTCCAGTGGATGGCTGGATGTCAGAGGTGGGAGGATTTG
GAGATAGAGGGGTGGTCCTTGGGGGGGGGGTGTTATGGGAAGATGAATACAACCCAAACCCATTGTATGCATGTATGAAACGCTCAATTAATAA
TGAGATATATATATATTGGTTTTTCGAGACAGGGTTTCTCTGTATAGCCCTGGCTGTCCTGGAACTCACTTTGTAGACCAGGCTGGCCTCGAAC
TCAGAAATCCACCTGCCTCTGCCTCCCAAGTGCTGGGATTAAAGGCGTGCACCACCATGCCCGGCTAATAATGATACTTTTAAAGTGAAAAGAG
AGGAGTAATTCTGAAGCTAGACACTTGTGGGAGTTTTGTTTTATAGACAACAAATTCCCCCACCACAACAGAGAAGTGAGCAGAGCCAAGGGTG
AGCAGCTTGCATTTCTTTCTCACCTCCTTTGCTATCTTAGCTTGAGTTGATAAATTCAATTCTCTCCCAATCTCCACGGGAATTGGTTCTAGTC
TCACTTAGATTAGGGAGTCCTCACGTCCCTTAGGGAAAATGGCCCAATGTTTGCAGATAACCTATATACATCTTCACATGGGCTTGAAATAATT
CTGATTCCTGAAAAATACCTATGACAGCGCAGATGCGAGCTTTGTAAATAGTAGGGACATTGCATTGTTTATGGAACAATGGCAGAAAAGAAGA
GTGTATTGGTCACTTTTCTCAATGCAGTCCCCAAAGACCAGACAAGAAGCAGCTTAGGTCATGGTTGGAGGGGTGATAGCTCATACTGGAGAGG
GAAGCCATGGCAGTGTGAGCATGTATGACAATTGCTTGCTCACACCGCCCTAAGGCCAGAAGCTCCAGAGGAACAAGAAGTAGAGACGAGCTAT
TAACCTCAAGACCAAACCCCAGTGACCTACTTCCTTCAGCTAACTCCCACCTCATAAAAGTTTCACAACTTCCCGAACAGCAGCAACCGGGGAC
CAAGTGTTCAAACATGGAAGCCTGTAAGGGACATTTCTCTTTCAAATAACAACCAAAAGTTTATGCTGGTGTCTTTTAAATGTCTGCTCTGGGT
TATTTATTGTCAGATAAGCTTACCTCAAGCTGACTTTTCTACTCATGGAGAAGGTAATAGTAATGGAATCATAAGGAAGGGGAGTCTAGAGCAG
GGGTACCCAGGCCTCCTAATCCTGACACCCTGGAATACAGCTCCTCACCTTGTGGTGAGCCCCGACCATTAGTTATTTTCATTGCTACTTCATG
ACTGTAACCTTGCTCCTATTATGAATTGTAATGTAAATATCTGATATGCATGATAGGTAGGAAGTATATCAGGTTCTAGGGATTCCAGGAACCT
CCTCAGAACTCTTTAACCTTGTCTGTGTAGATACAATTGTTTCCACCCCTGACCGGTACTTCTGAGGTGAGCTTAAAACATCTAATTCTGAGCC
TTCAAACATGGAAGACTCTGGGTAAGAACCCAGTCTTACCTTCTCTGACAGATCTTATTTCCTATATTATAAATATATAGGGAATATATAAAAT
ATGAAGTATGTGTATATTTCATATTTTAAAGCCCCCCCCCAGTTTAAGTATATTTTGTCCTACATCTTTTTAGCAATGGTCTATCACCAAGATC
TGCTAACCCAGTGAAGGTTCCAGCTCCATACTCTGTATGGATGTTTTCCCTGAAGGGCATTTTGAGAAGAACACAACATGGCTGTGTCCGGATG
GGTTCACACATGCTCTCTCGTTGCACAGCTTAGCGACCCTGGCTCTGGCTCTGTTTGAGGCTCTGGATAGATTGTCATGTGGAACTAGCTGCCG
GGGGACTCTGGTACTTGTTGCCAGATGAGCATCCATGGGAAGGTACGAGATGGAACAATGTGGTGTTGATGGTTTGAAGTGTGACTTTAACAGT
GCACTGTTGAGTTTTTATCTTGTCACAGTTAGGATTACTATTGCTGCAATAAAACCCCATGACCAAAACGCAAGTTGGGGAGGAAGGGGTTTAT
ACAGTTATATACTTCCATATCACTGTTCATCATCAGAAGAAGCCAAGACAGGAACTGAAACAGGACAGAAACCTGGAGGCAGAAGCTGATAGAG
AGACCATGGGAGGGGTGCTGTTCACTGGCCCGCTGCTCCCATGGCCTGCTCAGCCTTCTTTCTTATAGAATCGAGGGCCACCAGCTTGGGGATA
ACAACACCCACAATGGGTTGGGCCCTTCCCTATCAATCGCTAATTAAGAAAATGACTTATTGGGCTGGAGAGATGGCTCAGCAGTTAAGAGCAC
TGACTGCTCTTCCGGAGGTCCTGAGTTCAATTCCCAACAAGCAAATGGTGGCTCACAACCAACTGTAATGGGATCTGATGCCCTCTTCTGGTCG
GTCTGAAGAAAGTGACAGTATACTCACATACTCAGCTGCAGTACAACTGGGTGGCAGTTTTGCAACCAACAGTGATTAAGAAATACATTGTGGG
GCTAGAGAGATGGCTAGGTGGTTAAGAGCACTGACTGCTCTTCCAGAGGTCTTGACTTCAATTCTCAGCAACCACATGGTGGCTCAGGATCACC
TGTAATGGAATCCAGTGCCCTTTTCTGTTGTGTGTGAAGACAGCAACAATGTACTCATATACGTAAAATAAATAAGTAAATAAAACAAACAAAT
CAACCAATGAAACAAACCCCACTGCGTCACCAGAGACCATGTGGGTGTACGAGTGCAGCACTGCTGTGCCCATGGTGGTATCCAGGCCTATGCT
GCTGCCTGGAGTCATGTCTAGGACTGTAGTCCTGTCCCAGACAGGGTCTGTGTTGATGTCTGAGGCCGATGTTACCAATAAAATGTTGTGGGAT
GCCAGCCTACGGCCTACGTGAACCGGGTATTTCTGGAAGGCAGGCTGGGGCTGAAAGAGACTAGACTGTGAGAAAAGAATGATGAAGCCAAGAC
AAATTTTTTCTCTGATCAAGGCTCGTGAGTTTACTAAGAGAGTGTGCTTATAAGGGGGAAGGCCCATGTCCTCCCCCGCGCCCCACCCCCCAGT
CCATCCTTGCATCCTTGGTGCTTTGTCGCCATGTCGCCATGCTGTCAGCACTTGGTTGCTTATTCAGGAATGTCTCAGGAAGATAGGTAGCAGA
ATCTCAGGGCAGTTGACACTTCAAAAGAAGACAGTCAAGTCAGAAAGCTGCACCGCAGGTGGCCCCACCTCAGTGGTGACAAGGTCTGTACCAG
CCTGCTTCAGGTTCAGGCTGGGGGAGACTACAATAAAAGGCCATGCAGACGACCCTGGTCTGGGCTGCCTGAGGCCACGTTGATGTTCAAGCAC
TATGCTGAGCTGGCAACCATACCATCCCTTACGTAGGCAGTGTGCGAGAGACGGCTCTGGTGCCGTGGGTTCAGAAGAGCTGACCATGGTGGCA
GGAGGGCAGGAGATGGTCCTGCCCCTCACCAGCTGCAGCCCCCAGGTAGGAGAGTAGGCCATGTGCCTCATCTGGGCAGCAGAATAGAGATTAT
CCTGGTGATAGGGGAGCAGGCATACTGGCCCCATCACTTGTCTGACATGGGGCAGCATGGGTAAAAGAGAAATGCCCCCTTGCCCCTCACCAAC
TACTTCAGGTGGAAGGGCTGGCCCCCAAATCATGAGAGCAGGAGAGCTGACCCTACTTCTCACTGGCTACAGTACCCAGAGAGGGTGTCCTGCA
CCTTGCCTGGGTAGCAGAGTAGAGCTGGGCCCCTGCAAAAGAGCTGGCCCTGCACCTTGCCTGCTGTAGATTGGGTGAGCTAGCCAGGAAGTGT
TAGAGAGCTTGCCCTGGTGGTGAGTGTACAGGAGAGCAGAGAGGCTGACCAACTCATCTACCACCCAGGCTCAGATGTAGGGTTTTGAATTGGC
TTGACCTCAACTCTGAACTGCTGGAACTGTAGGGATGCTCCTACAAATCTAAAGCTGCAGGGTCTCCATGACACAGGGCAACAACAGGCTGTCC
AAAGGAGTCCCTGTAAGGGTCCAGCAGGAGCCAGAAGCCATGAACCAGACCAAAGTCTCATGGCAATGTTCATTTGCAAGTAAATAGAGTGGAC
CAAAGGGAGTTCTGTGGGACTGTGGGACAGACACACTATGACACACTATAGCTTCCACAGCAAGGGTTTTATTTTGGTTTTTTTTTTTTTTTTT
TTTGGTATTTTACTTTATTCTAGTTTATTTTATTCTGGGGTAAAGGTTGCAAGAGCAGAGGTAGATACTAAGGGACAGGAAGATGAATGGGGTT
GGAGTACATGATTCACACACACACACACACACACACACACACACACACACACAAATCAATAATTTTTTTTTAAAAAAACTATTCATTACAAAGC
CAAAACAAAATTCTGATTAAAAAAAAATTCTTTCTAAATGCCCCATTTGATAAGTTGGAGGTTTTTAAATATTCCATACTAAAGTCATCAGAGG
GGTTTCATGCAACAACTGATGGGAACAGATGCAAAGACCCACAGCCAGACACTAGGGAGCATTCAGGGAATCCTCCAGAAAGCAGGGAGGGAGG
ACTGCAGGAGCCAAAGGGGTCAAGGACACCGCAAGAAAACCACAGAATCCACTAAGCACAGAAAGCAGGGAGGGAGGACTGCAGGAGCCAGAGG
GGTCAAGGACACCGCAAGAAAACTACAGAATCCACTAACCAGGGCTCATAGAAGGTCACAGAGCCTGAACTGACAACCAGGGGGCCTGCGTGGG
TCTGGCTTCGGCCCTCTGCATATATGTTATGGTTGTGTAGCTTGATCTTCTCGTGAGACTCCTAACAGTGAGAATGGGGGCTGTCTCTGACTCT
TTTGCCGGCTTTTGGGACCCTTTTCCTCCTACTGGGTTGCCAGTCCAGCCCTAATAGATGGGGAGGTTCCCGGTCTTATTGCAAATTGATATGT
CATGTTTGGTTAATAACCCTTAGAGGCCTACCCTTTTCTGAAGGGAAATGGAGGAAGGGTGGATTGGGGGATGGAGTGGGGAAAGGAAATGAGG
GAAAGAGGAAGGGTAAACGGTGGTTGGGTGTAATATATAAGAAAAGAACAAAATTTTTAAATTATGTAAAAAAAAAAAAAACCAGGAATACATT
ATGGGACTATGAGCTGTCAGCAATGAAGCAGGAGCAATGACGTCTTTTGATGGCGTTGGCAGGCTCACAGCTAGAACCAGCGCAAGAGACAGGA
TGTGCTGTGGTTGTCAGAAGGGAGGGTTCCACACTCCTGCAGGCTGGTTGAGTGGAAGGCGTAGGGCTTCAAATCAGGGCTTCTGGGACCCGCA
TAGGCTTGGGTGTAGAGCGAAGAAAGGCCTTGTGCACACAGATAAGCACTGAAGAAGCCAGGAAAGGTAAACACACGGCTTGCCTCTTCGATGG
AATGAGGTGTGCAGATAGATGCTCTTCCTGGAACGCCAGGAATGATGGAGTTTTATAACCTGGCGATCCCTTGCTGTCTGATGAGACAGGCTCT
GCTCGTATCTCCCAGACTTCATTTCTTCCAGACAATACCCTCCCCCTTCAAACCTTGAGTAATGACTTTTAGACCACAAAACCCATCCTCATGT
GGGCACTGTCTCTGAGTCAACAGTACCTATTCCTGTGAAAGAAGCCAGATGTGTTTTGTAAAGAATCTCAGTGTAAGCATGGCTTAAATTATTA
TATACTCGGGACTGAGTTCATTGTTCTCTGAATACTTTTTTTTTTTAAATTGTGCTGTGCTTAAATATGGCTAAAGTAAAATGATCTGGGACAG
ACTCTAGAAATCCTGGTTCATCACCCGCTACAATCAAATCAGCCTGAACCAGGTTTTCCTCTTGCCTCACCCTGATCATTTTTTCCTTGCCTGG
TATAAAGCTTGCAGGGGAATGGCCACACGTGAGTTATGTGGGACAAATGGAATGTTTACCTTTCTTGGTAAGATCCCCTTCTTTTATGGTTTAG
CACTCATGTGATGTGGCACTTGAGTTGACCAAAATGATATAGAGCAATCAGAAGACTTTGAAATACCTCTGTGTCAGAAGCTTGGCATGTTCAA
GCTGCTGTGTATAGCACCACAATTCCAAACCCACCAGTTATGAAAACTGTTAGTGTTCTGTCTAAGCTCCTCTCCACAGTTACCTGGCAACAGC
TAGGTAGGCCTGATCCACTATAAAAGGGGCTTCTTGCCCTCTCCTCTCTCTGTTGCTCCTCTCTTACTCTTACTCTCTTGATCTTGCTCTGTCC
TCTTGTCACTTCTCCCCCCTCCCCCTCTCCCTCCACATGCTCATGACCAGCCTCCTCTCTCTCTCTCTCTCTCTCTCTCTCTCTCTCTCTCTCT
CTCTCTGCCTTTCTCTGACTCTACTACCCTCTTTTTTTTTTGTTTTTTGTTTTGTTTTATTTTTGTTTTGTTTTTTGAGACAGGGTTTCTCTGT
ATACCCTTGGCTGTCCTGGAACTCACTTTGTAGACCAGGTTGGCCTCGAACTCAGAAATCTGCCTGCCTCTGCCTCCCATGTGCTGGGATTAAA
AGTGTGCGCCACCACGCCCAGCCTCTACTACCCTCTTAACTCCCCTGACCATGCCCCAAATAAACTCTATTTTATACTATACTGTCCTGTGGCT
GGTCCCTCAGGGGGAAGGGATGCCTCAGCATGGGCCCTCAGAGGCACTGCTTTCCCCCATACCTGACTACATCTCCATAGAACATATCCCCCCT
CTCCTTATCTTTTTATAAACACATCAAAAACAACCCCAGGAAGCCTTTGTCTCATCCGTAAGAGATAGCAATCCTCAGTACAAAATTCGATGGA
AAAGAATGAACATGGGCTCTCAGACTTTTTCCCACGCCTACCAGCAATTGATCTGAAAGCAATTACTGTAGGCCCACAGCCTGGGTACAGAGCC
ATGGGACAAACTCGGGAAGTTCCACACTGAGGTCTGATTACAACTGAGCTCTGAATCAGCAATTAGATTTTCTTGACCCAGCAAATTTTCTCAT
CCGCCTTGGCGAGCCTGATTAAAATACCCATTTCTTCAGAAACGAAAATCCCAATTCCTATAAAATGATATCCTTGACCCGGAGGCACCTGCTT
GTGTAGGTCCGATAGGCTCAGTGTCTTTCTTCAGTGGGACTCCTTCAAGTACTCAGGACAGGGAAGACGTGGGAGGGGCCTGAGAGCAGAGGTG
ACAAACTCTGGTCTCCAGCAAGCAAGCATGTGATGAAAGGAGGGGAGGAGGCCTGAGGTGACCAGACAGGGCTTGGGCAACCTGCCAGCTCAAC
TCTAACTCTCCACTGGCAAGAAGACAGACATGGAGGTGTTTATGGAATGTCTGCTAGTTATTAAATGCTAGATAAAAAATTACCAGATTAGCCA
ACTCACACACTGGGGACCTATTTATAGCTTCATGGCTAGAAGTAAACAAACCGCTAACAGCGAGGCCCGAGGCATGGATCATTTAGGGTAGAGA
CATATTTGAGAACACCAAGAACACATAATCTCCTGAATGTCTCATCCTCTCGCATGCCCACGTGCTCTGCGGTATTAGAACGTCTTTGTTGAGT
CAACATGATCATGAGTATTTTGGTTTTTTGTTTTTTAATTATTTTTCTTTTATTTACTTTTTTTTTTTTTTTTTTTTGATCATGAGTATTTTGT
AACAGACATTTAACCATATAATCAGAAATGTCTCAGGCCTCCTTATCTGACATTCTGACAGTGAAGTAACTGGCAGAGAAATGAGATAGCTGGG
AAACCTGGTACCTGTTGTCCTGGGGCCAGGGTCTCTCCAGAGAAACAGAGCCTGGAGTGGGGGAGGGGAAAGCAGCAGAGGGAAAGAGACAGGT
AGATTCATTTTAAAGGGTCAGTGCCCATGACTGTGGGGAGAGTGAGGCTGACCTTTGTGCAGTAGGCCAGCAGGTCGAAACAGAGGCAGAGTTT
CCATATCACATTCTCCAGGCAGGGTTTCTTCATCTCTAGGAATTTGTTCTCAAGGTCTCACCTGATTAGAAGGAAGCTATATTGGCATATAAGC
TATGTAGAATTTAGCTGATACAACACATTAATTATAACTGTAAAGCGCATGGCAACATCAAGACTAGCATTTGATAAGATTGTGGGCCCTGTAG
CCTATCCAAGTAGATATGACAAATGAACCAAGAGATGAGGGAGCAGCCATCTTGGCCATGCACTTGGCTGAGAGCATAGAGTAGGCCAGCTTGT
TGGAACTGCCAAAGCCTTTAAAAACAGAAGTAAAGGGCCTGGAGAGAGCTCTGTGGTTAAGAGGGTTTGCAGCTCTTGCAGAGGACCTAAGTTC
AGTTCCCATTGTCTCTCTCACTGCTGCCTGTAACTCCTGCTCCAGGGCATCTGACACCCTATGAAGTGTTTCATTATAGTATAGCTGTCTGGGG
TCCATAGGGTGGGTGTTGGGTAGGGGTCCTAAAGTAGTGCTGGAGAGAAACTTAGAGTGGGGAAATGGGAGGGTGTATCATGAAAGGCTTTGAA
CCCTGCTGGTCCCGATACATAGATTACTATTTTTTTTAAAGGTGGTTTCTCTCTCCAGTCTACTTTGAGAGGTTATATGTATGTTCTAGTCAAT
GATTTATTTATTTTTCCTGTTTGTGGTCTCTTCTTCCCAGATCTGGACCTGCGGTCTTGGGAGTTGGAAGGATCTGATGGGAAACCCTCAAGAG
ACTTCTGGACTCAAAGTGAGAATCTTGCAGGACCTGCCATTTGCACTTTTGAACCCTTTGGACGGTGACCCAGGGCTCCGAAGAGGAGCTTGTA
AGACTGACAATCTTCCCTCTGTCTCAAGACTCTCTGAACAGCAAGACCCCAATGGCACTTTAGACTTACCCCTGGGATCCTGGACCCCAGTGAG
GGCCTAAGGCTCCTAATGACTTTCAGGGTGAGAACAAAAGGAATTGCTCTCCGCCCCACCCCCACCTCCTGCTTTCCGCAGGGAGACATGGAAA
TTCCCAGTTACTAAAATAGATTGTCAATAGAGTTATTTATAGCCCTCATTTCCTCCGGGGACTTGGAAGCTTCAGACAGGGTTTTTCATAAACA
AAGTCATAACTGATGTGTTTTACAGCATCCTAGAATCCTGGCAGCCTCTGAAGTTCTAATTAACTGGAAGCATTTAAGCAACACGTTAAGTACC
CCCACTGTGGTATTTGTTTCTACTTTTCTGTTTTTAAAGTGTGAGTCACAAGGTAATTGTTGTAACCTGTGATATCACTGTTTCTTGTGTCTCT
TCTTTCAACTACATCTTTTAAAACAAAACGGTGTGGGGTTTGGTTGTTTTGGTGGTAGTGGTAGTGTTTCTCAGTGGTATCTCCTTAAGAAAAA
AAATCATCATGCCAGTGAATTGTTTCTTCAGCCATTTCAGATGGGAAGCTGGAATAGCCTGTCCCCCAAGCTAAGCCTTCTTCCCTAGCTTTCT
GCGTGATTTTACATTGAGCATTCCTGTTGCTTTGTTTCTATAACTGTAATGTGGTGATGTCATTGTTAGGGCACTTGAGGGTGGGCGTTCTGGA
AGTCCTTTCAGGTTAGTGTTTGGGGGCAGGGTTGCTCAGAATACATAAAGGTGCTAACTTAAACTGCAGCCATGGAGCTCAGTGAATTCACTAA
CCTTCGGGCTGTCCAAATGTGCACATTAGCTACTGTGACCCCTGTAGGTTAGGGAGCCTGAAGCCAGCTCTTTACCTGGTGTTTAGACTCAGCA
GAATTTGGAGTCAATGGGACCAAATGGTTGTGAAATTAAGATTTGAAGTGTGCATCTTATTTTATCACCATCTGCCCAACAAAACTTCAGAAAA
TGCCTTTGAAGCACAAAAATGTAATCGTTTATGTGAAATCTCTGAGTTGCATTTAGATGCCCATTGCAGCAAGGTGGCTCTCTCACAGATTCCA
CACCTTAGCCTAAGATACCAGACAGCAGGACAGAGAGAAAAGTCCTTCCTGGTGTGCAAACTTCCTTACACTGGACCTCGCCTCTCAGGTGTGT
GATTGGTAGGCCAAATCCCGATAGCCAATCGGTGTTGGGTGCTTTGTCTGCTCTACTGGGAGTCCAGTGGTACAATGGATTCTGGCAAAATGCT
GCCATCTTGGCCCTCGCTGGGCTGCTTTCTAGGATATTCATAGAGAAAGGGCCGTCCAGATCCAGTATCCTAAAATCCTGAGAGGAGAATATAA
GTTAGTGTGTCTCACTATAACTATCTCTATGATCGGTCACATTACTATCTAACAGTTACCAAATACTATATGCCTAATACTGGTAAGCATTTTA
TACACACCATTGGATTGAATCCTCTCAAAATCCTCAAAAAGGAAGTTATTAATACCTCCATAGGCAAGGAGCCCAGAACCCAGAGAGGTCAGGC
AGTCTAGTTATAGATGCCTGCTTTGTTTAGAAGTGAACAAGAGCATCAAATTATTAATGTGCCCTGGTTATTAATGCGCCCTGGTTACCTGCTG
GATGGAACATCAAGGTGGACTTTTGGCAGTTGCATACACCCAGAGGTATTTTGGCTATTCACGGATTAATTTCACACGAAGTGTTTCAGAGACA
TGTGTAGGGGAAGTCCGGGTTCAGGGGGCCTAAGATTCAAAATCAAAAGAATGAGCAGTAAAAGAATGGTGTTTACTGCCTGCCTTTATCAGGC
AGTGAACGTGCAGCGGGCAACGAATGCTTGATAAGTGTGTGTCAGTGTGAAGTCCCATGTACCAGCCGCTGTCCCCACTGCAAAAGCAGCAGAG
CGCTCAGACATCATCAGCTGATTTACCAGCAGCAGATTTCTTCTTCTAGTCCCATCCCTGAAGAAGCTTCCAGCCTAGGTACATTGCATGGGCT
TTGTGCTCCAGGAGTTCCTACACAGCCCTCAACTTCAACACAGGCAAAGTGCTTACTGATCCTCATGTATCTTACAGGGTCCCCTCTACCCACA
ATACCTCATTGCTGGAACTTCAAATCTTCCTGAATAAAAGCTTGCCCGTGGTTTAATTATTACCTATACTAGGAGATTTTCCTCCAGCTGTCTT
TCCTTTGGACACAGAAATGTGCTCACTTTCTCTGTCTCTCTGTCTCTGTCTGAATCTGTCTGTCTGTCTGTTTCTGTCTCTCTCCCTCCCTCCC
TCCATCCCTCCCTATCTCTCATTCCCATCCAATGATGCTTACATGTTTTCGTTTTTGAACTCTGAGGACTGGAGCACACGTTATCTCCCCGCTT
GGACTCCTTGAAAGCAGAGAGAAAAGAGGATCTTCCCATGCCCAGAGCACCTTGTGTGAGATAGGTGCTTTGTGTGTGACTGACCCTGAAACTT
TAAATATTGAAAATAACATGACGCTCATTAGTCTGACTTTTTATGACCGTAATGTGATTGGCTAAAAAAGTAAATAATGGGAACTTTAAAAAAT
GTAATTCAAATATAATATTATTTCTATTCCTCCCTTCCCTTCATCTAACCCCTTCCATGGCTCCTTCCTCCAGTCCTCCTCATGGCTCCCACTC
GCTCTCAAATCAATGGCTTCTTTTTCTTTGACTACTACTGTCACTGACATGCACATAAATATATGAGCATAACTTGCTGCCTCTGTTAAGTGTC
GCTTGTGTTAATATGATTTTAGAGTTGACCACTTTGCATTAGGAAGTCAATTAGGGGGCCATCCCCGGGAGAGGATAACATTCCTCTTTGTGGT
CTTCAGTTGCCTGCAGTCCTTCACCTCGGACTGGTGACCTTTGTGACTTTCCTCTTCCATATTAGCAAGCCTAATTGCTGTTGTCCTTGATGGG
TCTGGTTCAGGCAGTCATGTAGTTGAAGAATCATGAACGTAGCTTCCCAGACATTTCTACAAGACGTGATCCCACAGCAGACTTTCCGGTTCTC
TGGTTCTCCGCACTCTTCTACTTCCATGATGTCCCTTGAGCGTTAGGTGCAGGGGCTCTATTATATACATGTCCGCTGGGTCTGCACCCAGATG
GTTAGTTTATCTCTGCATATGTAAAAGGTAATTTTAAAAATTTCTTTAACGTTTAAATTTTTATAAGTATGGGCATGTATATATATATATATAT
ATATATATATATATATATATATATATATATATACATGTGTGTGTATGTGTATATGTATATGTAGATGTAGATGTAGATGTAGATGTAGATGTTC
AAAGAAAGCCAGAAAGAGGCTGATCTATTAGAGGTAGACTTACAGGCAGCTGTGGGAAACAGGTCTTGGGTCCTCTGGAAGGGCAGGAAGTGCT
CTTAACCACTGGGCCATCTCTCTAGCCACTATAATGGCATTTTTTTTTAAAAAGTGGATTAATTCTTTTTAGTGTTTTATATATAAGTGAAGTA
AAAATAGGATTTAGAAGCGTTTGAAGATTTGAGAAAGAGCAATATAGGCTGCTCTCATTCAATCACAAATGTATTGAGTTCCATTACTTTTATG
TTTGTCTATTTATGAAGGTATACTTTATTATATTTCATGTAATTTTCTATCCTTTGAAAAAATCTGTTATGAATCTTAAGCTAGCACATATGCT
GCTGGCTAGTTGTAAGATATTCTTCGGGCAACTGTGACAGTGTTTGCTTAAACCTTCCTCAGCTTCTGGAAGATGGTAGCCTGTGACACACACA
CACACACACACACACACACACACACACACACACACACACACACACACAGAACACAGAGCAAACTCCATATGAAAGTCACTGCCCCCGCTACATC
TCTTTCACTACTTTTAGAGTGAATGTTAACCACATATTTCCTTGGAAATTCTCAAGTGTTCCCTAAGGTCAATTCTTACATATACACTGGCCGG
TGTGTATGGATCTTAACAGTGTTCTCCTGTTCAGTTCATGGGGTCAGCCATGGGGCAGATCAGAGGGCTAGAGAAGGCTGATCTAGACGTTTTC
TCCAGGCTTTCCCTCCAAGGGCCATTGCTGACTGTCTTGCCCAGAGCCCAGAGCCCAGAGCCCATGACCTTCTCCATGCGGCTTACTGTGCTGC
TGGGTTCCAGTAACTGCTGCCTCCCTTCCCCTTTTCAAGATTAGGTGCCTAGGTCAGTCTGTCTGTCTTCTTGTCTGTCTGTCTCTTACTCTCT
CAGAGAGAAAGATATAGACAGGCAACCTGCCTGCCCTGTGGTACAATACTATATCCAGCCCACACATTTACAAAGACTCCCTTTAATTAAAAGT
TCACATTTTCCAGTTCCAGACCTTGGCTGGTAGGTCAAGCTATTTCAAAGAATGTAATTTCTTTTTCTTAGGTAAAGAGTGGGTACTCTAGTTA
TGCACTGCTAGGATTGTTCTGGAATCTGTGCCCATGCAGACTACCTTCACTAAGAACTCGGAAAATACCCCTGAATTTGTGCTGTTGTTATTGA
TATTGTTATCATGGGTGTGGCGAGCACAAGGGTCCACATAGCGCAATGAGATTTGAAGTTCTTTGAAATGTACTTCCTGTTTCAAGGTATTTTG
TGAGCATGAGCTGTTTTATGACCTTGTAAAAATAGAGAATCCAGGTGGTGAGGTGCAGACAGCCAGTTAATTGTATTTGTTTTGTGACTCAGGA
GATCACCTCACTGCCATCAGAAGGGCACCCATCCTCTTAACACATCTGAGAGTGTTTACAGAATGAGTCTGAGGCTTAGCAGGTACAAGTGCTT
GTCACACAAGCCCGGTGACCTGAGTTCAAACCTCAGAACCCACAGTGGAAGGAGAAGAGAAAACCCACCTCTGAGAGTTGCTCTCTGACCTCCA
CATCTGTGTCATGGTGTACACACAAACACATGCAAATTACATATAAAAGTTGCTATCTTGTGTCTTAGTTGTTGTGTCTTTATTACTTTTAGAG
TGAATGTGAACCAAGTATTTCCTTGGAAATTCTAGAGCATTCCCTAAGGTCAAGTCTTACACATTGATTTCTGATTGATTGGCTTGGTTAGTTT
TGTTTTGCCATGTAGTGCGGTTTTGTTATCTTGACTTCTTCGGGGTGGTTACAGGAACACAAATTTGTTGGGAAGTTGAGCGTCTCAGGCTTCT
TAGGTGGACCACTTACTTTCTGGCGTGCTTTGTATTCACATTGCTACTGGATGCTCTTCCAACCCTGGGGATGCTTAAGGCTTCACTTTGCCTA
TGTAACAGGTTAAAGCAGTGATACTTTTACGTTTTCCTGTTTTGATCACCCCTGGCTTTGATCCTAAGCATCACTCTTCCTGTGACCTTGGAAT
CTGAGTGTAGTCTATCCTCCCCTTCCCTGCTCAGCCTCTAGCTAAGTGTTTCATTTACTCAATAATTTAATCATTTTGATACATTATTTTGTTT
TAAGCTACAGTGTCTTCACCTATCTATGGACTTCCTAACAATAATACCCCTCTCACTGAACTGTCCTATGAAGATGATGCATACCTGATTTAAA
AAAAGAAAAACCTATCCAACAAATACAGAAGTGGATGCTCACAGCCATCCATTGGACTGAGCACAGAGTCCCCAATGAAGGAGCTAGAAAAAGG
ACACAAGGAGCTGAAGGGGTTTGCAGCCCCATAGAAGAAACAATATGAACTAACCAGAACCCCCAGAGCTCCCAGGGACTAAACCACCAACCAA
AGGGTAAACATGGAGGGACCCATGGCTCTAGGTGCATATGTAGCAGAGGATGGCCTTGTGGGACATCAATGGGAGGAGAGGCCCTTGGCCTTGT
GAAGGCTCGATGTCCCAGTGTAGGGGAATGCCATGACAGGGAAGTGGGAGTGGGTGGGTTAGTGAGCAGGAGGAGGGGTAATAAGGATAGGGGG
AGTTTCAGAGGGGAAACCAGGAAAGAGGATAAAATTTGCAATATAAATAAAGAAAACATCTAATAAAAAAACTGTTCAAGAAACATGCATTTCT
TTCCTTAATAAATATGAATTAAGAATGAATAAATTATGTAAATTTTGAACAAATGATTGTGTCAATTTTCTTTGAAGTTTATGTTAAGAGTTAA
CTCATAAATTAGTATTTTGATGATCTAGATAGTCCAGTGCTATCTTATTTGTGGCTTTAGATTCTTCACTGTCTTTGATTAAAATCTTTGAATA
ATTATACTTTGAAATTATTTTTATTAGTCAAGATATATCTCAGTAAAATAAACTCTGACTTACAGCAAAGTTGGAATAGAGGGAGAAGCTAGAT
TCTCACTTGTAGTGAGTCATATCTGGTTTCTAGTCTGTAATTCAAGGTAGGCTTTTCAAATGACACAGACATTAATTCATAGAGCCATTCCCCA
CCAGGATTCCCGTGGGTTGACACATTCTGAATGCTAGATGATAGCTCGTCATTTCTATTGAAATCAGCAAGTCCAACTCCGTGGTGATGTTTCC
CACCACACCATGCTTAGAGCCTGTTCACACTGCTCTGAATCAGCTCACTGACTGAATTCTCAGTACTTCAGAGAAGCCACTACTCCCTGTTCCT
CATGGTGACCCAAGGGGCAAGACAAGATACATAATTATACATGACCAGGTGGTGAGTTTGCATCACGGAATATAGCCTACTCTTATGCTCATAT
TCCAACCACTCAACCAAGAAAGTAAAACCTTTTCCAACTGCTAACATATTTGTCTCATAGCTTTTGCTGTCTTACAAGCTACCCCAAACGCAGA
GATATCTGCAAGGTGCATAATAAACAGACACAGACTACTGGCAGGCAATTTTTCAAGACTTAAAGTTGCTCTCTTCTCTGTCTCTCTGTCTGTC
TCTCTCTACCTGTCTCTCTCTGTCTCTCTGTCTCTTTGTCTCTCTCTCTCTGTCTCTCTGTCTCTATCTCTGTCTCTCTCTGTCTCTATCTCTC
TGTGTTTCTCTCTTTGTCTCTGTGTTTCTCTCTTTGTCTCTGTGTCTCTCTGTATCTGTCTGTCTCTGTCTGTCTGTCTGTCTCTGTCTGTCTC
TCTGTGTCTCTTTGTCTCTTTGTCTCTCTCTATCTCTGTCTCTCCCTATCTCTGTCTCTCTCTGTCTCTGTCTCCTTCTGTCTCTCTCTGTTTC
TCTGTCTCTGTGTCTCTCTGTATCTCTCTGTCTCTGTCTCTCTGTGTCTCTCTGTGTCTGTCTCTCTGTTTCTCTCTCTGTCTATCTCTCTGTC
TCTCTGTGTCTCTCTGTGTCTCTGTCTCTCTGTTTCTCTCTCTGTCTATCTCTCTGTCTCTGTCTCTGTCTCTCTCTCTGTCTCTTTCTCTCTC
TGTCTCTTTCTCTCTCTGTCTCTCTGTGTCTCTCTGTGTCTCTGTATCTCTCTGTTTCTCTCTCTGTCTCCATCTCCCACTCTCTGCTCTCTCT
CTCTCTCTCTCTCTGCTTGAGACTGCACACATTCTGCATTCTCCTGCGCACTCTGCTTTTCTCCTGTGCACTTTTCTTTTCTCGAACTCGCACA
CTCCTACACACCTCTGTGCGTTCCCCTTTCACTCACACACACACCTCACACACATCTCACACACACCACATGCACTCTCCTTTCACTCACACAC
ACATTCTTCTCTTCACACTTTCATATTTTCTCCATTCACTTCACACTCTGTCCACACACACCTCACATTCTCTCTTCACTCTCCTCACATTTGC
TCTCTCTCTCTCACACACACACACACACTTCTCGTGCACCTCACTCCCACTTCACTCTCTCTCCACTCTCTCTCCACTCGCTCTCTCGCCTTTT
TCTTGCCCCAGTCTTTTACTGATACTCCAAAAGCAGGGAGAGAAAAGACATTGTATTCTCATTGCCAAATCAAATTCAAAAGAATAACCACATC
CCACAAAGAATCAAGAATTACAGTTGTTCCCCAAAGTTTCAAGCTTACCCCTATTTCCAGGCTTGTGGCCAAAATAATAATTGCAAAGTCATCA
CTATTTAAACTTTAATTTTTTTTAACTTATACTTCAGAGCTCAGAGGTCCGAGGTCAGCTACGTGTGTTTTCTTGTGAAGCTCTAATGATAAAT
GACGTGGGCAAAGCTGGCAGGCAGTGGCCAGAAAGAATCAACCCTTAACCCTTAACTCAGCAGTTCTGCTAGCTTTCTGCTTCTGCACATTGCA
CACAACGACTCTCCTAAGAGTCTCAGTGTTTTGACTTAGTTTTACTAGTATAGAATTATATGCATTCTACAATTGCTTATCCAGGAACCACTCT
CAAATGTTGTGCAAAACTTATTTCATAACTTCAACAGCTTTGCCTTCCTTGGGTTTCCAATGTTGGCTCTAATTCATTTTCTAATAATTTCTTC
AAACTTTCTTTGCAAGTCAAGCATTAATCTGGAACTCCCATTGTGCAGTTGTTACATTGTTTCCAAGATGAGAACACACAGCAGGCACCTGGGT
CATTAGGACTGTGCTGTGCTGTGCCCCAAGCCTCAATTTTTAATTGTGTAAGAATCATTGCATCTAGGAACATCGAGTTTCACAGAATTCACCA
GAGCAGGAGAAAGGAGTAGGAAAGTCAGATATAATAGAATAATTATGCAGACCAAGGCCAACCGAAAAGCAGTCACGAGCAGATGAATTCTATA
CAACTGTTGTCCAAGGCTAAGATTAGGAGAAATGGGAACAACTGTTGTCTACAAAAGACAGCTGTGAGCTACTGACATGTCTCCACCCTGGCCT
ACTGCAGGCAGTCCCTTATTTCAGGTGGCAGGTCCCCTGGAGGGATGTAACTACTCCTTCTCAGGTTCCCAGGCACCATCCTTTTTCACAAGGA
GCTGCCACAGGCTTCTGAGATGTCTCCATCCCTTTCTGCCCCTTTCCCTCTGAAAGAGACGGGAATCCTGGAGTTTATGAGAAATGGAGACACC
CCTTTCGAGGCCACTGCTCATATGTGGTTGTATCAGAGTTTCAAGCAAAAGAAAGTTAGTGTCTGCAGATGGGAAGGCGGGACTTCCTTCATTA
TGAAGGGGAGATCAGGGACAGACTCAGGACAAGTGTGCTCAAACATTCACTCCTAGCCTGAGTCCAACGCCTTCCAAGTTTCTGTTCTCCGTGC
ATCTGCCAGCTGCGGAATGCTTGGTTTGCTTCTGAATCCTTAGTAATATTTAGTTATTCCTGCAACCAGAAAGAACACGATATATTTTGAACGC
CTGCTTAGAACCCCCTACTGGTGCTCTGCCTACGGTTTTAGGGGAAATCTCTGAGCTTGCTGCTTTCTTCCTGCACACACACTAATACAGAAGC
AGCCACATCACCTCGATTCCTTTATGACCTTATCTTTACTAGAACAATGCTCCAATATAACAGCTACTCGGTTTCCAAAAATTCTACTTCTCCA
TCAAGCCTCCCTCGGGTCTCAGGAAGCTACGAGGAAGAGGAGGCAGGAAAACCGTAAATCGGTGGGGATGGACGACACCAAGGAAATAAGACCT
TCTAGATAAGAGAGCAGAACTGATGCTCGTAGAAGCTCACAGACACCGTGGCAGCACGCACAGGACCTGCACAGTCCCAGTACTGAGAGGGGAA
CTGGACACGAGGCCCATCCCTCAACGCAGAAATGTTCTCCAATGAACAGCCATTCACAGAAGAAAAATCAGTTTTCTCCAGTGGAGTCTCACTG
GGTGTACACACAACACTTAAGGGAGGTTCCATGCCCAGCAGCAGACTTGCAAACACAAAGCAAACTGCATGGTAGTTTTTGGAGATGTTATCTA
GCAATACTTTGTTTGGTCTGTTTTTTTGTTTTTGTTTTGTCTTTCCTCTTTATACCTTGTCTTTTGCTTTTCTATTATGATCTCTGATTTTTTT
GTGTTTTTATGGGTTTTGTGCTCTGTGTGTGTGTGTGTGTGTGTGTGTGTGTGTATTCATGCAAATACATACAAGCTTTTTCTCTCTCTTTTTT
TTCTGTTTCTTTGCTTTTATTCTGGATTGTTTTATTGTTTATTGGCTTATTTTAAAAGGTCTGGGGTCATCTGGGTGGGCGTGAGAAGGGTCTG
AGAGGAGACGATGGAAGGGAAACTGTGATCAGAATACTGTAGGAAGAAGTATTTTCAATTAAAAACAGTTAGAATGGAGCTGGCATGGTGGCGT
ATGTACGTGATCCCCGCACTTAGGAAGCCAAGGCACAAGAACCTCGTGAGTTGGGAGCCACCCTGGGCTATAAATTGAAAGCCAGTCTCAAACC
AAAAGAGGAAAATCATATGCAAAGTGGCTTATGTACAAAAGAAGGACTACTCATTTGTGTAAATGAGCAGCCTAGAGGTGCAACTGGTCCCATC
AGAAAACC
MOUSE SEQUENCE - mRNA
ATGCTGCTCCTGCTGCCGATACTGAACCTGAGCTTACAACTTCATCCTGTAGCAGCTTTATTCACCGTGACAGCCCCTAAAGAAGTGTACACCG
TAGACGTCGGCAGCAGTGTGAGCCTGGAGTGCGATTTTGACCGCAGAGAATGCACTGAACTGGAAGGGATAAGAGCCAGTTTGCAGAAGGTAGA
AAATGATACGTCTCTGCAAAGTGAAAGAGCCACCCTGCTGGAGGAGCAGCTGCCCCTGGGAAAGGCTTTGTTCCACATCCCTAGTGTCCAAGTG
AGAGATTCCGGGCAGTACCGTTGCCTGGTCATCTGCGGGGCCGCCTGGGACTACAAGTACCTGACGGTGAAAGTCAAAGCTTCTTACATGAGGA
TAGACACTAGGATCCTGGAGGTTCCAGGTACAGGGGAGGTGCAGCTTACCTGCCAGGCTAGAGGTTATCCCCTAGCAGAAGTGTCCTGGCAAAA
TGTCAGTGTTCCTGCCAACACCAGCCACATCAGGACCCCCGAAGGCCTCTACCAGGTCACCAGTGTTCTGCGCCTCAAGCCTCAGCCTAGCAGA
AACTTCAGCTGCATGTTCTGGAATGCTCACATGAAGGAGCTGACTTCAGCCATCATTGACCCTCTGAGTCGGATGGAACCCAAAGTCCCCAGAA
CGTGGCCACTTCATGTTTTCATCCCGGCCTGCACCATCGCTTTGATCTTCCTGGCCATAGTGATAATCCAGAGAAAGAGGATCTAG
MOUSE SEQUENCE - CODING
ATGCTGCTCCTGCTGCCGATACTGAACCTGAGCTTACAACTTCATCCTGTAGCAGCTTTATTCACCGTGACAGCCCCTAAAGAAGTGTACACCG
TAGACGTCGGCAGCAGTGTGAGCCTGGAGTGCGATTTTGACCGCAGAGAATGCACTGAACTGGAAGGGATAAGAGCCAGTTTGCAGAAGGTAGA
AAATGATACGTCTCTGCAAAGTGAAAGAGCCACCCTGCTGGAGGAGCAGCTGCCCCTGGGAAAGGCTTTGTTCCACATCCCTAGTGTCCAAGTG
AGAGATTCCGGGCAGTACCGTTGCCTGGTCATCTGCGGGGCCGCCTGGGACTACAAGTACCTGACGGTGAAAGTCAAAGCTTCTTACATGAGGA
TAGACACTAGGATCCTGGAGGTTCCAGGTACAGGGGAGGTGCAGCTTACCTGCCAGGCTAGAGGTTATCCCCTAGCAGAAGTGTCCTGGCAAAA
TGTCAGTGTTCCTGCCAACACCAGCCACATCAGGACCCCCGAAGGCCTCTACCAGGTCACCAGTGTTCTGCGCCTCAAGCCTCAGCCTAGCAGA
AACTTCAGCTGCATGTTCTGGAATGCTCACATGAAGGAGCTGACTTCAGCCATCATTGACCCTCTGAGTCGGATGGAACCCAAAGTCCCCAGAA
CGTGGCCACTTCATGTTTTCATCCCGGCCTGCACCATCGCTTTGATCTTCCTGGCCATAGTGATAATCCAGAGAAAGAGGATCTAG
HUMAN SEQUENCE - GENOMIC
AATGGAGCCTGTGGGCTGCGTTGCGAACAATGCCGGCTTCATTAACATAAAAACAGCATTCTTCTTGGACATATACACATGTGCCCCAAGACAA
GAATGTTAATCCAAACTGCACCATTTTGTAAGCGCCCTGCTATTTTGCAGACCTTGGTAAAAGTGACACATTTCATGGGTTTTTGGGCCATGAG
AAACACCCTGCCTAACCACTTGACCACAAGGCCCACAAAGGCCCAACTAAAGAAATATCTCTATCATATCCTTCTGGGCAAAGGTCCAAGGAAC
ACCACGATGACATCCCGCCTGAACAAGGACCAGAACTGCCTCATGACGGGAACATCTTATCAATATCCTACCGGGCAGCAAGCCATACTGCCCA
GACCCCTCCCGCCCATACCTATAAATTACCCCAGCCTGTAAGCAGCGGTAGGCACTGGCGTTAGGCTGGTCCCCGACTTCTACAGTGTGCATAT
TTCTTTAACCCTCGCCTTCCCTTCAAAACCTAACAGGGATCAGCCTGGGCAATATGGCAAAATGCCATCTCTGCAAAAAAATACGAAAATTAGC
TGGGTGTGGTGGCGCATGGCTGTGGTTCCAGCTACTTGAGAGGCTGAAGTGGTAGGATCGCTTGAGCCCCGCAGGTTGAAGTTGCAGGCAGCCC
TGATTATGCCACTGCACTCCAGCCTGAGTGACAGAGTCAGACCCTGTCTCAAATAAATAAATAAATAAAAATAAATTAGAAACACATACAGCCC
TTTGTTGATTTCTTTAAATGTTTTCTTCTTGTTATTTTTCAGTAATCTAACTATACTCTCCATTATTGCTTTTAAAAAATTATTTCTGTCTTTA
CAAAAGTAATTTGTGCTCATGGCAGACTATTTAAAAAACAAAATGAAGCCAAAGAAAGAAAATAAAAATTAACTGCAGGAGAAGGAGGCAAGAG
AGCTAACTAGATGCAGCCAGGAGAAACACCTCTCACAGAGATCTTCCCAGATTTTCAGAGGGAAGGCATCAAGAGTGGACAGAGGGAAGACACA
GAGGCTGGGCTGAAGCAGGAGGAAGCTGAAAACCCTGCATGAGGCTACCACACACTGGGACTTGTTCCTGGCCCCCAACAACTCCTGCAGAAGG
GATGAGTTGAACAGGCAAGGAGAAACATGCTCTTGCAATGGGTCTCTGGAATCACGACAGGAAGAGACCCCTCAACCACTGTGGACACTTGAGT
TGGCAAGGAGAGCTGCTTAGAGAAGTGGTAGGGGCAGAGCTCCAGGTGATGTGGAGCCCAGTAGGTTTCGTGCGGGAGTGTTTGTGGTGGAGTA
CAGCCAGCGATGTCCATCTCCCTAGGCTCAACTTGCTTCCATAGGAGACTTTAGCACCAGGGGAGCTGTCGGTCCTAAATTCTGCAGGGCGGTG
TTGCCCATGAGATAGGGCCAGTCTGACTTCAGCACCCTTTGGTCTGCTAGCCTTTCCCAGGGCCCCAGCCTGTCCATGCCTGCTTGCAGTGCAG
CCACCAGGTACCTCCTGGGAGCCCACATCATAGCACCTGTGCTGGCAGACCATGCCAGACTGTCAGAGTGCTGCAGCAGAGTAGCCCCCATGGA
CACACACCAGCCCACCCAGCTCATCCCCTAACTACAGCCTCCCCCTTGTTGCTTTGCTTGCACACACTTGCCCACAGCCACCCTTCATATCACT
TTGCCAGTACCTGCGTGTACAGGCAGACCTCACCTTCCCTTCCCCACCAGCATACGTGTGAGCGTGTGCCCTGCCATGCCACTGCTGCTGGTGT
GAGTATATCCTGCCCCTCCTTTCCCTGCAGCACTGTCCACTGTGATGGCAATGGCATTGTCATCAGAGTGTTGTTGGGCATGGAGCTGCCAGCT
CCGCCCCAGCCAGTCCCCAGCCCTGCCCCTATGCGAACACTGCCTATGGCACAAAACTAGGCACAAAAACCAGCAGGCCCACCCCTGCCCTGAG
CGGCCAATGCCTCCCATGTGAATGTGCACAGGGGGCACACACAGCCCTGCATCTAGCAGCATCCTGCTCCCATGCTAATCCCAACACTGGCACA
AACATGTGTACAGTTGCTGGTGAGGGCCCCCCAACCTGCCTGAGCCATGCTGCCACTGCTGCTTCTATGAACACCTGCACGAAGGCTGGCACTC
CGGCATCCACTAGCACCTTGCTGCAGCCAACAAGTGTGCACCCCACTGCACCGCTGCTGCCACTGCGACTGGCACATGCGACTGAGGATGGATC
ATGTTTCCACAGCCCTACAAAGCACTTTGGCTGGCACCATGCCTCAGAGAGTTGTGATCAGAGGTCCAGGAGCACCTCAGGCCCCTCCAACATA
GCAGGTTCCTAACCTTAAGGAGCCAGAGAACAAGACCGGGGCCTGATACCAGTGCCCCAGAGTTATAACACACAGTTTGGGAGTCCTGAGCTGA
GACTTGGGCCCCTAAAATCTTCCAGAAATGAAGCCAGTCTACTGAACCCACCTTATACCACAATCAAACCCCGAAGGTCATTAAATATGATATA
AAAAGGGGGAAAAAAAAACAAAAACAGCAACATCAAAGATTGAAGGAACATCATTCTACAAAGATGAGAAAGAACCAAGATAAGAACTCTAACA
ACTAAAAAGCCAGATGGTCTTCTTTCCTCCAAACAATCACACTAGTTCTCCAATAATGGTTCTTAACCAGGCTGAGATAGCTGAAATGACAGAA
GTAGAATTCAGAATATTGATAAGAACAAAGATCATCGCAATTCAGGAGAACACTGAAACCCAATTTAAGGAAGCTAAGAATCACAATAAAACAA
TACAGGAGGTGACAGACAAAATAGCCAGTATAGAAAAGAATGTAACTAAACTGATAGAGCTGAAAAATATACTACAAGAATTTCATAATGCAAT
TGCAAGTATTAATAGCAGAATAGAACAAGCTGAGGAAAGAATCTCAGAGCTTGAAGACTGACTTTCTGAATTAAGACAGACAAGGAGACAGTCA
GACAAGAACAGAGAAAAAAGAATAAAAAGGAACAAACAAAACCTTTAAGAAAAATGAGATTATGTAAACAGACCAAATCTATGACTTATTGGTG
TCCTTGAAAGAGATGGGGAGAATGGGAGCAACCTGGAAAACATATTCCAGCATATCATTCATGAGAACTTCCCAACCTAGCTAGAGAGGCCAAC
ATTCAAATTCAGGAAATGCAGAGAACCCCCAGAAGATACTTCACAAGACTGTCCCCAAGACACATAATTATCAGATTCTCCAAGATGGAAATGA
AAGAAAAAAATGCTAAAGGCAGCTACATAGAAAGGAAAGGCCATCTACAAAGGGAAGCCCATCAGACTAATAGTAGACCTCTCAGCAGAAATCC
TACGAGCCAGAAGAGATTGGAGGCCTATATTCAACATTATTAAAGAAAAGAAATTCCAACCAAGAATTTCATATCCAGCCAAACTAAGATTCAT
AAGTGAAGAAATAAGATATTTTTCAGACAACTCAACGCTGAGGTAATACATTACCACCAGGCCTGCCTAACAAGAGTTCCTGAAAGAAGCACTA
AATATAATAAGGAAAGACAGTTACCAGCCTCTACAAAAATGCAGTTAAGTATATAGACCAGTGACAGTATAAAGAAACCACACAAACAATGCAC
AGAATGGCAAGCTGGATAAAGAAGCAAGACCCAATGGCATACTGTCTTCAAGGGACTCATATCACATGCAGTAACACACATAGGCTCAAAATAA
AGGGATGGGAAAAAATTTACCAAGAAAATAGAAAACAGAAAAAAGCAGGGGTTGCAATCCTAATTTCAGACAAAACAGACTTTAAACCAACAAA
GACCAAAAAAGAAGAAGAAGGGCATTACATAATGATAAAGAGTTCAACAAGAAGATATAACTATCCTAAATATATATGCACCCAACACAGGAGC
ACTCAGATTCATAAAGCAAGTTCTTAGAGACCTTCAAAGAGACTCAGACTCCCATACAACAATAGGGGGAGACTTCAACACCCCACTGACAATA
TTAGACAGATCATCAAGTCAGAAAATTAAAAAAGATATTCAGGACCTGAACTTAACACTGGACCAAATGGACCTCATAGACGTCTACAGAACTC
TCCACCCAAAAACAACAGAATATACATTCTTCTCACTGCCTGCCACATGGCACATACTCCCAAATCAACGACACCATCAGACATAAAACAATCA
TCAGCAAATGCAAAAGACCAAAATCATACCAACCACTCTGTAGAACCACAGTGAAATAAAAATAGAAATCAAGACTAAAAAAAAAATTGCTCAG
AACCATTCAATTACATGGAAATTAAACAATCTGCTCCTGCATGACATTTGGGTAAATAATGAAATAAAGGCAGAAATCATTAAGTTTTTTTAAA
CCAATGAAAACAAAGACACAACATACCAGAATCTCCAGGACACAGCTAAGGCAGCGTTACAAGGGAAATTTATAGCACTAGGTGCCCACATCAA
AAAGTTAGAAAGGTCTCAAATTAACAACCTAACACCACAACAAAAAGAACTAGAGAAGTAAGAGCAAACCAACCCCAAAGCTAGCAGAAGACGA
GAAATAACCAAAATCAGAGCTGAAATGCAGGAGAATGAGACATGAAAAACCATTCACAAGATCAATGAATCCAGGAGCTGGTTTTCTGAAAAAA
TAAATAAGATAGATAGACTGCTAGCTAGACTAATAAAGAAGAAAAGAGAGAAAATCCAAATAAACACAATCAGAAACAACAAAGGGAATATTAC
CACTCACCCCGTAGAAATACAAATAACCATCGGAGACTACTATGAACACCTCTATGCACACAAACTAGAAAATCTAGAAGAAATTTATAAATGC
TTGGACACATACACCCCCAAGACTAAACCAGGAAGAAACTGAATCCCTGCACAGATGAATAATGAGCTCCATTACTGAATCAGTAATAAATAGC
CTACCAACCCCCACCCCCAAAAAAATGCCCAGGACCTGATGGATTCACAGTCAAATTCTACCAGATGTACAAAGAAGAGCTGACACCATTCCTA
GATAAATTATTCCAAAAAAATTGAGGAGAAGGGGCTCCTCCCCAGGTCATTCTATGAGGCCAGCATAATCCTGATACTAAAACCTGGAAGAGAC
ACAATAACATCAACAAAAAACTTCAGGCCAATATCCTTGATGGACATTGATGCAAGAATCCTTAACAAAATACTAACACACTGAATGCAGCAGC
ACATCAAAAAGCAAATCCACCATGATCTAGTAGGCTTTATACCCAGGAGGCAAGGTTGGTTCAACATACTCAAATCAATAAATGTGATTAATTA
CATAGACAGGACTAAAGACAAAACCACATGATTATCTCAATAGATGTAGAAAGGTTTTTTATAAAATTCAACATTTCTTTATGTTAAAAAATGC
TCAACAAACTAGGTATTGAAAGAACATGCCTCAAAATAAGAGCCAACTATGACGAACTCACAGCCAACATCATACTGAATAGGCAAAGGCTGGA
AGCATTCCCCTTGAAAACCAGCACAAGACAAAAATGCCCTCTCTCACCACTCCTATTCAACATAGTATTGGAAGTCCTGGCCAGAGCACTCAGG
CAAGAGAAAGAAAGAAAAGGCATCCAAATAGGAAGAAGGGAAGTTAAACAATCCCTGTTTGCAGATGADCATATTCTATATCTAGAAAACCCCT
AGTCTCAGCCCAAATGCTCCTTAAGCTGATAAACAACTTCAGCAAAGTTTCAGGATACAAAATAAATGTACAAAAATCACTAGCATTCCTATAC
ACCAGCAACAGCGAAGCCGAGAGCCAAATCAGGAACACAATTGCACTTGCAATTTCCACAAAAAAAAAAAAAATACCTACGAATACAGCTAAAC
AGGGAGGTGAAAGAGCTCTACAATGAGAATTACAAAATACTGCTAGAAGAAATTAGAGATTACACAAACAAATGGGAAAACATTCCATGCTCAT
GGATAGGAAGAATCAATATCATTAAAACGACCAAAGCGGTTTATAGATTCAATGCTGTTTCCACCAAACTACCAGTGATATTCTTCACTGAACT
AGAAAAAAATATTTTAAAATTTATATGGAACCAACAAAGAGCCCAAATAGCCAAGGCAATCAAAGCAAAAAGAACAAAGCCAGAGGCATCATCC
TACCAGACTTCAAACTATACTACAAGGGTACAGTAACCAAAACACCATGGTTCTGGTACAAAAACAGACACATAGACCAATGGAACAGAATAGA
GAACCCAGAAATAAGGCTGCACACCTACAATTATCTGATCTTTGACAAAGCTGACAAAAACAAGCAATGGAGAAAGGACTTCCTACTCAATAAA
TGGGGCTGGGATAACTGGCTAGCCATATACAGAAGATGGAAACTAGACTCCTTCCTTACACCAGATACAGAAATCAACTTAAGATGGATTAACA
ACTTAAATGTAAAACCTAAAACTATAAAAAACCCTGGAAGACAATATAGGCAATACCATTCTGGACATAGAAGTGAGTGAAGATTTCATGACAA
AGACACCAAAAGCAATTGCAACAAAAGCAAAAATTGACAAATGGGATCTAATTAAACTAAAGAGCTTCTACACAGCAACAGAAACTATCAACAA
AGTAAACACACAACCTACAGAATGGGAGAAAAGTTTTGCAAACTATGCATCTGACAAAGGTCTAATATCCAGTATCTATAAGGAACTTAAACAA
ATTTACAAGAAGAAAACAAACAACCCAATTAAAAAGTGGGCAAAGGACATGAACAGGCGTTTTTCAAAAGAAGACATACACATGGCCAACAAGC
ATATAAAAAAGCTCAATATTACTTATCATTAAAGAAATGCAAATCCAAATCACAATGAGAGATCCTCTCACACCAGTCAGAATGGCTATTATTA
AAAAGTTAATAAGTTGGCCGGGTGCGGTGGCTCACACCTGTAATCCCAGCACTTTGGAAGGCTGAGGCAGGTAGATCACAAGGTCAGGAGATCC
AGACCATCCAGGCTAACATGGTGAAACCCCGTCTCTGCTAAAAATACAAAAAAATTAGCCGGGCGTGGTGGCGGGCGCCTGTAGTCCCAGCTAC
TCAGGAGGCTGAGGCAGGAGAATGGCATGAACCCAGGAGGTGGAGCTTGCAGTGAGCTGAGATCTCACCACTGTACTCCAGCCTGGAAGACAGA
GTGAGACTCCATCTCAAAAAAAAAAAGTTAATAAACAACACGCTGACAAGGTTATGGAGAAAAGGGAATGCTTATACACTATTGGTGGAGTGTA
AATTAGTTCAACCATTATGGAAAGCAGTGTGGCAATTCCTCAAAGAGCTAAAAACAGAACTACCATTCAATCCAGCAATCCCATTACTGGGTAT
ATACCCAAAGAAATGTAAATCATTCTATCACAAAGACACATGCACACACATGTTCATCATAGCACTATTCACAATAGCAAAGACATGGAATCAA
CCTAAATGCCCATCAACGACAGACTGGATAAAGAAAATGTGGTACATATACACCAAGGAATACTATAGAGCCGTAAAAAAGAGCATGTCCTTTG
CGGGAACATGGATGGAGCTGGAAGTCATTATGCTTAGCAAATTAATGCAGGAACAGAAAACCAAATACCACATGTGTTCACATATAAGTGGGAG
CTAAGTGATAAGAACACATGGACACAAAGAGGGGAACAACACAAACTGGAGCCTAGCTGAGGGTGGAGGCCCCAGGGAGGAGGGAGATGAGCAA
AAAAAAAAAAAAAAAAAAAAAAAAAAAAAAAAAATGACTGGGTTCTAGGCTTAGTATCTAGGGGACAAAATAATCTGTATGACAAACCTCCATG
ACACGAGTTTACCTATGTAAAATAAAAGTTTAACAAAAAACTAAAAAAGTTACCTATGTAAAACAAAAGTTTAACAAAAAACTAAAAAAAGTTA
CCTCCACCCAGAAATGCCTTTATTAACACTTTGGTTTGTAACCCTCCATTATTTTCTCTAGACATATAGATTTATGAGGCAGGCTAATGTGCTG
AGACACTAGCTTGAGTTTCATTTGGCCTTGCTGTGAATATACCTGGGCCTTCCTTTGCTTCAGGATGCCCTAAACTGTGGCAAAGCAGAGCTGA
ACTGCCCAAAGGAAAGTGTGAAGAGGAGAGGCAGGCCAAGCCTGGAGAAAGGCTACCCAAGAGAGAGGAAAGGAAACTGTGGTGAGTGCACCTC
ACTCCTAAAGAAGGACAGGAACTCTGGTAGGTGAGGTGTGTTTTCCATAGATACTAAAACTCAAAGCCACACAGGGGACATATGTCCATTCCTC
AGCCAGTCACTGTGGCCAGGAACAGAGGGTCAGGTAAGAGAATGGCAACTCCTAAGGAAACCACATGCCTGGAGCCGTGGGTGTAGAAGCAGAT
CCCCAGAGGAAGCACGGGTCCCACAGACACAGTCCCAACTCTTGCCGTCTAGGCAGCCACTCCCATCTGTAGGTACTACACAGCTTCGTAGTAC
TTCTTAAAGTACTTCTTGTTCAAGCGATGGGACGAAAAGAGGATGCAGGACAAGGAGGAGGAAAGGAGATCCTTGCTTTTGTAGAAGAAACAAG
GAGGCAGGGAAGGCTTAGAGGACAGGCAAGGAGAGATGTTTTCTTAAAAACATCCTCTGTCTGAGTGTTCAGCCCCACATCTCTGCTGGTCCTA
CCCCTATAAGCACAAGGAAAAGGGCGGGAGGCAGCAGGAGAAGGATTGATCATTCAGGCCTATATTCAAGTTTGCCTTTTCTTGCCATGTGGTT
TTAATCTCACCAATCACAAGCCTGAAGGTATAGTGGGGCCACTGAGGTCCGAGTTCAAATTCAGCCATGCAACTAAGGAAACATTGTTTGATCT
TGGTCCTGTTTTCTCATGTGTAAAATGGGTCTAACTCTAAACCACACATTGCACTGAGAATGAAATGAGGCACTATGAAATGAAGTGAGGTTAG
TTGAATAATGTTCCCCCCAAATTCATGTCTACTGAGGACCTCAGAATGTGACTATATTTGGAAGTAGGGTCTACGCAGATGTAGTCTAAATGAG
GTCATACTGCATTAGGGTGGGCACTAATCAAACGGATGGTATCCTCATAAGAGGAGAGGAATGTAGAGATACACACAGAAAAGAATGTCATTTG
AAGATGGAGGCAGAGATTGGAGTGATACAACTATAAGTCAGGGAATGCCAAGGACTTGAGGTCATGTGTTACAGAAGCCCTAGGAAATGAGTCA
ATTTCCTGGCACAGCACTAAGACATGCTGGTATTCTTCATTCCATCCCTTTCCCCTATTCCTAAATGTGCTCCAGGGACAATGGGGAAGCTGGA
GCACATATAAGAATAGGGGAAAGAGAGGGAATAAAAGGGTATTAAAAACTCTCAAGGTATGTTAAAAAAAAGACTCTCAACAAACCAGGTATTG
AAGGAACATACCTCAAAATAATAAGAGCCATCTATGATGAACCCACAGCCAACATCACACTGAATAGGCAAAAGCTGGAAGGATGCCCCTTGAA
AACCGGCACAAGACAAGGCTGCCTTCTCTCACCACTCCTATTCACCATACTATTGGAAGTCCTGGCCAGAGCAATCAGGCAAGAGAAAGAAAGA
AAAGGCATCCAAGAAGGAAGAGAGGAAGTCAAACTACCCATGTTTGCAGATGACATAATTTTATATCTAGAAAATCCCACAGTCTCAGCCCAAA
AGCTCCTTAAGCTGATAAACTTCAGAAAGTTTCAGGATCCCACATGAACATACAAAAATCACTAGTATTCCTAAACACCAACCATAGCTAAGCC
AAGAGCCAAATCAGGAATGCAATCCCATTCACAGACAAACTGCTAAAAGCAAAACCAAAACTTTCCAAATAAGCCAGGCTTTCGTCAGTTCCTC
AGAACTAGTTCTGGTTTGACTCACTCTCATGTTACGGCAAACCTTAAGCTGAATGAACAACTTTTCTTCTCTTGAATATATCTTAACGCCAAAT
TTTGAGTGCTTTTTTGTTACCCATCCTCATATGTCCCAGCTAGAAAGAATCCTGGGTTGGAGCTACTGCATGTTGATTGTTTTGTTTTTCCTTT
TGGCTGTTCATTTTGGTGGCTACTATAAGGAAATCTAACACAAACAGCAACTGTTTTTTGTTGTTTACTTTTGCATCTTTACTTGTGGAGCTGT
GGCAAGTCCTCATGTGAGTAACGAGTGGGTTGAGATACTCTCATACTATTATGTTGTGGACCATGATGAGGCTGCCAGGGTGGGGGAAGGGTGG
CCTACCTTCTCTCCACACCATTTGAGGCAAGAGACATTCTGCCTCAGAAATTGTGCTGAGGGGCACAAATAAGCTAATGTCCTCCTGCCCCAGA
TTTCTCTCACTTGTTCCTATGCATATTGTTCTTGTTGCTCAGTTTTATAAGGTGAGATATTTTCTGCACCAAATATGTGCACTGTAAAATAATA
TAACTATTCTTAAGATTGTCTCTCAGCTTAGAGTTTTCGGAGGCTTAATGGTGAAGAACTTAAAAATCTGTTTTCCTATGACTGACATCTGTTT
GTGATTATCCATTTAGCAGACGGTGATCCCTCCAGGCTGGCCTTTGTTTACAAGCTATGCCTAAGAGGCAGCTGTTGGTGGAGGGGGAACAAAA
ACAAAAAACCCAAAACTATTATCTTAATAACTTATAGAGGTAATCATTATTGAACAAAAGCATGATTTGGGGACTATGGAAAGGCTTATTCTTT
CCATATAAAATGATCAGCGAAAAGGTAGAAAAATTAAACAGAACTGAAATAATTGCTATTGATTGCTCACCGCCTGGATGCACTGAGTAGAAGA
GCAAAGGCAATGGGTTCATGTGGAGGGGATGGAGGTGGAGAGGCTAAGCCTGAGTGAGATTACCAACTAATCAGGACTTTTAACCCAAAAATAC
TATGGCTCTGAGGGGCAGGGAATGAGAGACCTAAATTGACAAAATGCAGAGTAAAAATATTCAGTGTGAGCAGTAATGAGACAGTTGTATCAGG
GTCAAATACGGAGTTGGCCTGGATGTCCTCCAAGCTCCTTTCCAAGCCTAAGATTCTGTGAGTGGATGAACAATCCTGGTCCTGCTGCTCTTGC
CACTGGAACCTGATCTATTTGCCTTCATTATTTGAGGTCTCCCCATCATCAGGCTCTTTGTTGTCTGCTGCTTCAGGGTGTCTTCTCCCCAGAT
TTCTCCTGTTGGTGAGCTTTTCCATTGAACTGGAGCTCCTCACAGGCAAGAAGCACACCTTTAAGGTTCTCTGAGTCCCAAGTTCCTAGTGCAG
TACCTGGCAATACACATAGGTGCTTAGTGGGTGCTTGCCGAATTGTTGAATGGATTTATTTGATGAAATGTCAGGAGAATCACCAGAAATGAAA
TGTTCGTGTAATGGACAGGGGGAAGTTTGAGGTTGATTTGAAAAAGGAGATGGTAGAGACAGTATTATGGGTCATGCTTCAGAGTTTATTTGCT
TTACAATACATTGGACAGCAACCAGTAGATTGTTGTGTGTCATTTTAATTCCACATGCTAATGTCCAAATATGTCATATGTCTTAATTGTTGCA
TACGTGGGTTACTCTGTAAAGGGGCAACTGTAGTATCACCTCCTCATGCCACACCCTGCTCCACTGTAGCTGGGCTCGATAACCTTCAAATCAA
CTCACAGAATTCCAGGACAGACAAGAGCATCTGAAAGGTTATCTAGCCCCAGTCACCTCTGATGCCTTCCAGCTGTCTCTAACACACCTGTGGG
CTGACTTCCAGTTCTTAGTCTTCACGGCTCCCTATGATGAACTGCCCCTGCTTTGAGGGATTTTCCTCCATTGCCTCAGAAGTACTGCCCTGGG
GGAGCTTCTGATGCTCTTCTGACCACCTTGTCTCTGCCTTTTTCACTAGTCCTCTGTTCCTGCTCTCCAAATTTTGGGACGTTCCTGAGTTCAG
ATCTTTTCTTTATAATCTTTCTTTTATATTCTCCCCATCAGATGGTTCATTCATTCCTCAATATTAACTAACATTTCCATATTAGAAATAACTT
CCTTATCTTTAGCCCTAAATTTTGTCCCAAACTCTGGACCTACAATTTTTTGGAATGCTTATGCCTTGCCCAGACTAAAACCAAACTTACTGTC
TTCCCTCTAAAATCAGCTCCCCATCTTCTATTTCTCCCTGCCACCCTTCCTCCCCCATGGCTCCAATATTGTCTTGCCAGTCTGGTCTTAAAAC
CACAGTCAATCTTGTTGCTTTTCTCTTTGTCTCTTGTGCATGGCTGATCAGCAGTCAATTGTTTTGTCAATTGTCTTATATGTTTCTTCTTCCT
CTTCCTTGCTTCCTTTTTCTGACCTGAAGTTAGGGCCTTGTTACCAAACATCTAGATGAATACAGTGACCCCCTAATTGAGAGGCTGTCTACCA
ATTCCACCCACCCTCAATTCTATACTCAAAAAAAAAATTAGATGTAATCTTTTTCAGATACTCCTTTGGTATTGTCACTTAGTTTTTGAGTGAC
TGCCACTATTTCCCGATTGGCAGCTCTGCCTGGCATTAAGCTCTTGACATTCTCAACCTAAGTGTCCAGCCAAGTTACTCTGTTCATTGTCCCC
TCAACACGCCTGTGCATTTGCACCTCTGAGACTGCTTCTGAGTCAGCTATGCACTGCCCTTTAGAATAGCTTCAACCCCAAATCCTACCAGTGC
TTCAAAGGCCCAGTTCAAATCCTACCCCTCTGGGAAGTCTCCCTTCCTTTAGTCAACTTCTAGGGCTTTTGCAATTTTATCACTTGTTTTACAT
CAGTTTTACTATTTTTCATGTGTGAGAGTTTTAACCTTCCAATTATGGTACAATGTCTTTAAGGGCAAGGGCCCTTTCTTAGAGGAAACCCTCT
ATGAATTCTCCAATGATAAATTATTCTATGAATTCTCACAACTTTCTAATATAAAGGCTTATAAAAAGTGGGAACTCACTGTTTATTGCTTAAA
AAATTGAGATTTAATTTGAACTTAAATGCTCTATTAAATTGAGTAGAATAGCATCTTAAGGCTACTGGTAGTCTATCTATGCCACAGATGGTTT
AGAGATCAAATAATTACTCATATACCTATGACAGCAGAGTACTGGCTGTAAGATCCCCCCAAAACTCAAATACCTATTGGGTTTGTCAGGCAAC
AAAAATAAGTGAATCCAGCTAGGTCTAATACAATGGAAAATGGTAGGACCTGGGGCAAACTCTGCATAACTTGCCCAAAGGCATTCAAATTCAG
ATTATGTTCAGACATTGCTAATTACAATTAGTGTCTTAGACACTTGGCTTCAAAACAGTGGTTAGTTCAGGTATTTCAGATCAATTGGGTATGC
ATTTATCTAGGTCTTCATGGATAGCCGGGCTCTACTAGACACTGGGAGAGGGAAGAGGCAAATGGGATAAGACTGGCAGTATTTAACAAAGAAT
GAAGTATTTTACAAACATGATCCTTTAGGCTGGTTATTGTTACACAAAGGAATGATTTGCTAGGATTCTTGAGATACTTCCATAGAATGTCTAT
GCCTCACTTATTGCACAGACCAGAGAATTCCATAAAAGCACTGGCTGTGTCTTATTTACCCTTGTATACTAGAGCTTGGCACATAGTAAATACC
AAGTAAATGTTTTGAATAAAAGAAAGAGATAATTAAAGCTTCAGCACCACAATTATTTGTATTTCATCTATCAGTTTGGAAATAGATCTCCACA
GACAGTAGAAATGGCAAGAAACTAGGCCTATAGCAACCAAGAAGTGAAACTGCTAGAACCTACCATCATTTCTGATCCTCTTGCAAATTTTGCT
TGAAAAAAATTTCCTCATTTTCTGTTTACGAACTTATCCTCAGAATTTACAGATGTTTCCTTGATTTCTCTGGAAAGATACAACTTCCTAATGC
AATTGAGTAAATTGATACTTCTTATGTAGAAATAAGCAGAATAAGCTGGGCAAGGTAGCCCACAACTATAGTCCCAGCTACTTGGGAGGCTGGG
GCAGGAGGATCACTTGAAGCCAGAGTTTGCTTGAGGCCAGGAATTCGAGGCTGCAGTGCTCTATGATCACACCTGTGAATAGCCACTACACTCC
AGGCTAGGCAATGAGTGAGACCTAGTCTCTAAAGAAAAAAAAAAAAAAAAAAAAAAGACAAAATAAGATCAAAATTGAGTATGAACTGATAAAT
ACAAGGTAGACAAGAAATATCCTGAGAGGCGCAATCGTTCTGCAGACATTTGCTTGGGAAGCTCAGGGGAGGATTTATGAAGGAGGTAGCCCTG
AGTGAGTCTTAAAAAATGGGTAGACTTTTACCAGGCAAGATCTTAGTGAAGTTAGTGAGAGGAGTGGTCATATAGGAATTCATGGGATTTTTAG
ACCAAAGATTACCACCCTCATAAGTGATAATGACGATGAATGTGAAGAATGTTTACCACTTGCAAAGTATTTTTCACATACCTTGTCCAAGTTC
CCACAAGCATTTCTGTTCACTACCTGTGGTTTGGTCAAGAGACCCAGATATTAGAGAACGTGTTGATTTCTAGTTTCTAACTCCGCCCAATAAC
ATTAAGATGAAAATGGATAGCCACATAGGAACAGACATCAATAAACATATTTAGAAAACACAAGTGAATTTTAAAAGGGGAGACTTAAGAGGTG
GCATGGTTGTTTGAACATGTTCGTTTTTGTCTCCTGGAGAAAAGTCAGCACAATGCTCATTCAGCTACATAAGTGTTTCTGTTACAGCTCTTTC
AGTCCACCATTCGGCAGGTCCTGAATTCTTGTCCCTTGTCCAGAAAAAATGAGGTATGTGGACAACTGGAGGGTGAGCAAGGCGGAGAGGAGCT
TCGTTGAGTGACAGAACAGCTTTCAGGAGACCCAAAGTGGGTAGCACTTTTCCATAGGCAGGAAATGAGTGTTCAACTCTCAGCAGAGAGGAGA
CCCACAATGGATAGCTCCTTACCACAGGCAGGTCATCCCAGAGAGGTGAGGAGACACAAGCCAGGCACAGTGGCTCACGCCTGTAATCCCAGCA
CTTTGGGAGGCCGAGGCGGGTGGATCACGAGGTCAGAGTTCGAGACCAGCTTGGCCAAGATGGTGAAACTCCATCTCTACTAAAAATATGAAAA
ATTAGCCAGGTGCAGTTGTGCACGCCTGTTGTCCCAGTTACTCAGGAGGCTGAGGCAGGAGAATCACTTGAACCCGGGAGGCGGAGGATGCAGT
GAGCCAAGATCACACCACTGCACTCCAGCCTGGGTGAGAGAGCAAGACTCCATCTCAAAAAAAAAAAAAAAAAAAAAAAAAAAGAAAAGAAAGA
AAGAAAGAGGAGATCTGAAGTGAGCAACTGCTTCCCCTAGCTGGTAGTCCCAATGTCTGTTCAAGTCTGGCTGAGTCCAGGGTTTTGTGTTTGT
TTGTTTTGTTTTTTTGAGACGGAGTCTCACTCTGTCACCAGGGCTGGAGTGCAGTGGCGCAATCTCGGCTCACTGCAACCTCTGCCTCCCGGGT
TCAAGCGATTCTCCTGCTTCAGCCTCCCGAGTAGCTGGTATTACAGACACCCACCACTACATCCAGCTAATTTTTTGTATTTTTAGTAGAGACG
GGGTTTCGCCATGTTGGCCAGGCTGGTCTCAAACTCCTGACCTCGTGATTTTCCTGCCTCGGCCTCCCAAGTGCTGGGATTACAGGCATGAGCC
ACTGAGCCCGGTCGAGTCCAGGGTTTTTACAGGCTCAGAAGGGAGGAAGAGTGTGCTGATTGGCCCATGGGCAGCCATGGAAGGGCCCAGAAAA
AGCACTGTAAGTTCTTACCCCAGGCTGTGGACTCCACCCACAATTGGCAACCTGGCTCCCAGGCTTTGGGCCATCCCTGGCTTGAAGGTTGGGT
TTCACCAGGGACCTAACCCCTTTCCGCCAAGGAACCTGTCTCCCTCTTGCCATCAACATGCTGTCCATGATGCCCAGGCTGTTCGTGCCGAGGG
GTGCCTGCAGGCCTGCCCTGAGCCACCCTCAGCCCCTGCTCAGCCTCCCTCTGTGCTTGTCAGCACCCAACATCTGGAAGAGGTCGAGGCAGCA
GGGGGCTAGTGTGTCAGTACCATCCTGAGCACCCACATGCCTGGCCGGGTTGTGACAGCACCTAGGCTTGGCCACAACTTTGCTTCACCCAGTA
GCAGGTGCCAGGAGTGGGAAGAGGCCAGGGAGTGGGAGCAGGCACTTTCAACCTTGTGGGAGCAGGGGACTTCCTGGGCCCCTGAGAGTACAGA
GATGCCTGGGTCCAGAGCCACTGCTGGGCAGCTACAGGGGTGCCTGGGAGCATGCGGTTCCCACCCCGCCAACTCAGTACGGGGCGGGACTCCC
ACCTGTTCTTGGCCTCCCTGGCCACACCTCCACTGCTGCAGCTGCTCTAAACGGGCAGCTGCTGCCATCACTGTGAGCTATGATTGTGCCACTG
ACTCCAGCCTGGGCAACAGAAAGAGACCTATATGGTATGTTAAATGGTAATGACTGCTTTGGGGGAGAAAAGATGACTAAGGATTGTTGGGAAT
GCTGGGGTAGGGTTGGAGTGCTCTTGCAATTTTACATAAGACAGTCTCAGGAGAAAAAACAATGAAAGTGTCCGGGTAGAGGGACATGTACACT
GGCAGCAAGGTCTTCCCTTAGGGATTGGATCTGCGGAAGAGGCCACACAGGCCTGGAAGCAGGCTTAAGGCCATTCAGGCTAGGAATTCCAGTG
TTTCAAAGACAGACTGCAATGGATGGCGGTTGGTAGGTCAAAAGCTCCAGATAACATCTCTCAGCCCTGCGAACACCTTACTCTCGGGGAGAAG
GATGGGCACAGTGGGTCCTTTAAAGCATGGGGTCCAGGACAGGGGCCTCTTGTCTTGGCTGTTGAAAAAACTGGGTTTTACACTGAGTGAAATG
CTGAGCCATCGGAAGGTTTTAAGCAAATATGGTTTGACTTTTATTTTTTAAAAACCACTCTGGCTGCAGAATCAACTGCAGGAAGGCAAAACTG
GAATCATAGCATCCAGTTGTCAGACTAATGCAGAAATCACCATGAGAAATGACAATGACTCTGACCGATATGGAAGCAGTGGAGGTGGTGAGAA
ATGGTCAGATTCTGGATATATTTGAAGGAAGGGCCAATAGGGTTGGTTAATGAATCAGATGTAGAGTGAGAAAAGAAAGGACTCAAGATGACTC
CAAGGTTCTTGTCCTTCACCTCAATAAGTGGAGGATGGAGCAGAGGAAGGCAAGACTAGAAGGTAAAAATAGCAGTGAGAAGGCTCTGCTATGG
TATAAGAAGAAATGATAAGAAGCTGAATTAAGGTGATGGCAGTGGGGTGGAAGAAAGGAGAGCCACCATGCAAAAAGTATCCAGGAGGGAGAAT
TAACAGGACTAGGGGATGGGCCATATTTGCAAGATGAGAAATGCAGAGGTCTAAGATTCTAGCTTAAGGTGGGTGTTGCTACTATTAGCATAAT
AGGAAAACAAGGCACAAAGACTTTTCAAAATTTGTCCAAGGTGTTAGAGGCTTACAATTTGTAAAACCAGGATTAAACCCAGATGTGTCTGATT
TTAGAGCCTGAGCTCTTACTCATTGCATAAACCATATTTTCCCCAGAGGAGGATTAGTAGGAAAGGAAGCTGCTGGTTGGAAAGTATCTTTATA
GCAGTGTCTGTTCCTCGGTTTGCTCAAGGGGACAGTGTGCCAGGAAAGTCCCCGTGGAAGGGCAAGGAAGAAGGGGAAGTTAAAGCCAGTGGCA
GGTGATCCAAGAATCTTTTCTGTTGCTAGAGCTATGTTACATGCTGTCCTTTCATGCTCTAAAAATAAGAGTGCTGGCAAGTGCCAGGCCTGTT
GGTGCAGCTTAAGATGATACCTTTCTTGGATATATATGCATCTGAATAAGGAAGGCTATCTTCTGGTCAAGCTAAGGTATGCCATGAGCATTTC
CCTGTGGAAAGCACTTAATTCTGTTCCCAGTTGTTACCTGCTGTAAGATCTCCCTTTCTAAAATAAAAACAAGAATACAGCTCACTGAGGACCT
TACATTTCCCTCTAGCTACTGACTCATTTCTCTTCTCCTTTTTATAGCACTCTTCTTGAGAGAGTTGCCTATATTTGTTGCCACATCTTTACCC
ATTCTCTTTTGAACCTATTCAAGCTTTCATCTGTACAAAACTCACTGATACTGTGCTTGTCAGGATCATCCATGACCTCCATACTGCTAAATGC
AACTCTCAAGAGTATTTGGCTCTACTGATCACTCCTTTGTAGCACTGTGTTTTAAAATATAGGTTTTATTATTATTTAGGTATGGTGAGGCCAA
TATATCAGGAAATGACTGTCGTTGAAAAAAGTATGTTGTACTCACAGATCCCAAGAGAAGGGGGGCACACCATGCCACAAAGGGCCACATGGGG
AAGCACCAGGGTCAGCCAGGAGGTGGGTGGGGGGTGCGCAAGATCTTTATTGTGGTTTCAACAGGAAGAAATGGGTGAAGCAGGGTGAGTGGAT
TTAGGATTAGCTGATATAAATAATTTCAGCAGGCTCTGGGGCATAGGGGCTGTCCCTAGTCTTCTGGTACTTGGCCCTGGGGTGATTAAGGCAG
TTGCATAGTGTTGGGAATGTGAAAGCCCCCAATAAATGAGGCAGTTGTGGGTATGGGCTCTGAAATGGGTTGGTTTGCATTTGAAAGGTGTGCT
CATGGGCAAGTGGTTTACTCTCTCTTAGAGGTTAGAATTGGCTAACCCTGGGAGCGGCAGTCCCTTCAGGGTCAGCAAGGCCCCAGGTGTCAAA
GCATCAGAATACAGAAAATAAAATGCATGGATAATACACACTGCCATTTGCCTTTGTACCCTTCCTTTCAATCTTCTCTGCTGGTGACCGCTCT
TCACAAAGATCTATAAATGTTGGAATACCCCATGTCTCAGTCCTTGGGCACTCTCTTTCCTATCTCTCTGTAGGTGATGTAATGCAGATATCCA
TGACTTTAAATCTTTAACACTTCTGCATTGATGACTCCTAAATTTACATCTCTACCCCAACTGCCTACTAAACACCTCCACTTGGCTATCTAAT
AGGCATTTCAAACCAAATCTACAACAAACGTAACTCTTTTTCCCCTTCCTTAATTTGCTTCTCCCCCAGCCTTCTCCATTTTAATAAACAGCAT
CTCCATTGCCTTAGTGACTCAAGCCCCAAACTTAGGAATTTTCCCAGATTTCCCTCTTTTTCTCAAACTATATATCTAGCCTGTCAGCAGTTCC
CTTCAGGTCTTTTTTCAAACTATAGAAGGCCTAAACAAAGGAAAGACATGCTGTATTCATGAATTGGAAGACTAAATATTATTCAGCTGGCTGT
ATTCCCCAAATTGATCTATGGATTCAATGCAATCCCTACCAAAATTCCAGCTTCCTTTGTCTGCAGAAATGAGCAAGTTGACCCTAAAATTCAT
TTGAAAATGTAAGGAAGCCCGAATAGCCCCCTAAAAAAAAATCTTGAAAAAGACTAACAAAGTTGGAGGACTCACACTTTCCAGTTTCAAAACT
TACTACAAAGCTACAGTAATCAACGCTGTGTGGTACTGACATAGGATAGACATATAGTTCAATAAAACAGAATCGAGAGTCCAGAAATACATCC
TTATATATATGATCAATTGATGTTTTGCAAGGGTGCCAAGACAGTTCAATAGGGAAAGAATAATTTCCTCAACAAATTGTACAGGCACAACTGA
ATGCCCACAATCAGTAGAACAAATTTGGAACTAAAGTTCTTATATTTAGGTTCTTGATCCATTTTGTTTAATTTTTGTATATGGTATGAGGTAA
GGCTCCAAATTTGTTCTTTTGCATGTGGACATCCAGTTGTCCCTGTACAATTGCTCCATTTATATTTATCTTAGCAGAAAATACAGATGTAAAT
CTTTGTGACCTTGACTAGGCAATGGTTTCTTAGGTATTACACCTAAAGCACAAACAATAAAAGAAAAAAGTAGATAAATTGGATTTTTTTACTT
AAAATCAATTTGTGTTTCAAAATTAAATAAAATCAAAATTAAATTAAAACATTTATCCTTCACAGGATTCTATAAAGAAACTGAAAAGACAATG
TGCAGAATGGGAAAAAATATTTCCAAATCATATATTTGATAAATAGTTTGGCAATTCCTCAAAAAGTTAAATATAGAGTTACCATTTAATTCAG
CAATTCTACTCCTACATATGTACCCAAGATAATTAAAAGTATACATAGACAAAGACTTGTATATGAGTTTTCATAGGAACTTTATTCATAATAG
CCAACAAGCAGAAACAATCCAAATGACCATCAACTGATGAATGGGTAAGCAAAATGTGGTATATCATACAATGGAATATTATTTAGTCACAAAA
AGGAACGCAGTGCTGATATATGCTACAATACAGATGAACCTTGACAACATTATGTTCGGTGAAAAAAGCCAGCCACAAAAGTCCACATATTGCT
TGATTCCATTTTTATGAAATATCCAGAATAAGCAATTGATAGAGACAGAAACTAGATTAGTGGTTGCCAAGGGCCATAGGGAAAGGGAAGGGGA
AATAGGAAGTTTCTGCTAATGGGTAAGGGGTTCCTTTTCAGGGTAATGAAAATGTTCTAAAATTGGATAATAGTGTTTGTTGCACAACTCCGTG
CAATTATACTTTAATAAAGCTGTTGAAATTTTTAAAATAAAATTTTAAAACAAAGAAAAAATATATAAACTCGCCAACAGACCACTTCTCACCC
CTACTAGCCCCTCTACTCTAAGCTATCAGCATTTCTGCAAACACATTCCTAACCGATCTCACTGCTTGTAATCTTGCCAGCAACCTCTCCCTCT
CAGCAATAGTCTATTGCCTACACCAAAGCTTAGTTGTCTCTTAATGATGTAAATGAGGTTCTATCATTCTCCTGACCCAAACCCTCCACTGCTT
TTCATCACACTCAGAGCAGCTCTGCTGTTGCCTGATTTAGATGTATGGCTCCAACAGATTTCCCCTGAAGAAATGATTCCATGGCTGATAAAAG
TTGGAAAGCCTCCTCAGTTTCAGACCATTATCAGATTAGCTGTGTGCTCTGTCCCTTTCCTCAACCATAAGAAGTCCATGGATAAAGAAAGCTT
CAGAGTAAAGGAGAAAGCATGGGAGGTACAGCAGGACCAAGGTGGGGCATTCGCAGCCCCCACCCTCATCAGAGCCAGTTCCCTACTCTCCCTG
TCTAAACCTCTTAGTAAGAGGTAGTTCAAGAGAGGGGCAAACTCAATTCCAGCACTCAAAAGCACTTGACTACTTTGCTCAGTCAACTAGCAAG
TATTTATTGAGAATGTAGCTCTGTTCTATGGAGTCTTATTTTCAAGTGTCAGACTCCCAGACATCCAGTCCAGGTAAAGAAGATGGTGTCCATT
ATTCATTTGACAAACAAAGTTGGGGTTCAAGGGCCAGCTATTGAAAAAAGCTATGGAAAGCTTCATGAGACGTGCAGGTAACTGCCAATATGTG
TGGTTCACAAGGACTGGTTCATATTCAGAAACGGCCATTAGAAAAGGAAGAAGAACTTCTCATTTGGATTTATAAAGAGTGTCTTGTTTACTCT
TAATTTATATCTTCTCTTCTCCAGGAAATCAACCTATAACTTCTCCTCCCAGCTCCACTCTACCATGGTCTGTCACCTTCCCCAAATGATTTGT
TATTCCCCTGTTTTCAAAAGTGAACAAAGAACCAAAGACCCAGCAAAGTTTCACAAGGCCCTGAGACTTTCAATTGTCTATTTCAGATCAAATA
CAGAACATGATCTTCCTCCTGCTAATGTTGAGCCTGGAATTGCAGCTTCACCAGATAGCAGGTAAGAAAGGACAAAGGGAGAGGCTTAAGAAAG
AAGAGCAGGTGGTGGTTCCTAGCCAAAGCCAAAAATGAGAATGTGGCCCTCAGGCTGAGGGCTTTCTTTGAGAGGACGTATGATTTCTGGGCTA
TTCCAAGCACCACAAAAAAAAAAAGAGTCCCCATGGTGGCTTATACATGCCAATGTCCCTATCTGACAGAAACGGTGACTGAGAATATTGCTCC
ATCTATTCCCACTATCCAGTGAGGGTAATGACAAGAAGACAGGATCACTCAGACCATGTAAATCTAAACTGATACAAGAGGGCAGGGGTTGAGT
TCCCTTAAAGGTGAGATGCCAAGCAGCTGTCCCCTTCCTTTCTGGCAGGGAGAGTAAGGAGACAATGGCCAGGGAACACCGTTACTCTAAAGAT
AATGTCTTGAAGACATTCTGCATATTATTAGTTGTTTCTGTGAGTTTCTTTTTTGAAAAGCAACAATAGCAGCCGTTGGTCATTCATACCTTAA
TGTGGTTTACTGAGTCTTCCTAAAACCCAAATGAACAATGAACCTTAAGGCTATCCCTTTGGACTTGAAGAAAGGACTTCTATTGGAGGATGAG
GGTGAGCAGAAAGAAAAGCAGTTTCACAGTTGGTTGTTCTCCTGGGGAAGGTAGTTCAGACCATTCGAGGGTGTAGTTAGAACCATGAGTGCAC
TATTTTGGATGAACACCAGGAGCTAAGAGAGTAACATAGAGGTGTGGACAGAGGATTAAGTCCTCAAGACAATAGCCCCAGCCCCATGGGAAAT
CATCTTTCTGCTCATGATTGAGAAATAATGGCTCCCTTGGCACTTGATAACCTTTCGAAGAGCTTTCTCCTCCCTACTAGCTGGTTCCAGATCA
CTCTTCACCCAGTCACATTCCTCTCACTCACTTGAGCTGCCCAGCCTGGTCTGGCACTAGAGACATGCACTTGGGGCCCTCCTCAAAGGAAGAC
CCTGAGATATTCTGCTTACTTCTACTCTGCTCCTGCCTGCAGGGCCAGCTAAAGGAACTTTTCATGTTTTCTTTGCAAGGAACCCTGCCTGGCT
GGCATTTTAGAGACAAGCAAAAGGGGCAATAACTTCCTTGCTACAAAACAGCTTCAAGTTTCCATAGAGTGATAAGGGAAATGAGGGCCAAAAG
ACACTGTTCCCCATCCTGTGGCAGGACTGGGGGCTTCAGGAGAAAACTTGGGGAATGTGTAACCTCTGTGGGTTTGTAGCTTAAAAACACTGAG
ATCCTGGGTTTTCTGTCTTTGTTTTTTGCCTTTTCTCTTAGGAAAGGAGTGAGCTAGGGTGACAAGGGGCAACATTTTTTATCCCTCATTGGCT
CTTTCTACAGAGGAAGGATCTTTTCTTCTAAGATAATCAGCACAAGACAATGAAGATAGGCACTAGCTCCCAGTTAGGTATACTAATGGGGCAA
AAGGAAGAGCATTTACATTTATTGAAGATTCACTAAATGCCAGATACTGTGCTAGGCAATTTACATATGGTATAGTTCATTTAATCTTCACAAT
GATCATTTTGCAGGTGAGGGAACTAGAACTCAGAAAAGGTACTTAATTTCCCCAAGATTACATAGTTATTAGGTGACACCGGCAAGATTTCAAC
AAAGCTAATGTCCTTTCTACTTTACTGTGCTACCATGATGATGGTAATCAAAAATGGCAGACAACCCATAAATCTTCCAACTTTGGAATAGGTT
TTTGCACTGAAGTCTGAATATGGATACGTATTGAATGTTTATTCTGGATATTCACAGAATCAAAAAATATGTGTAATGAATTATGTTGCTGAAT
TAACTGAAAGGAAAGTAAAAATGTAGCGCTTTCTCATTTTCTTCACGAATTTGGAATTCTTTTCTGCTTTCCACTATGCAGATAACATCAGTTC
AGACAAATATTAAATACCTACCTAAATTAGAATGCCTTCTCCTCATGGGATTTTTTTAAAATCTTGTCATTTCATGTCTCTTTAATTAAAGAGT
TTTGATTTCAGAGGAGGGTACCTGCAAAAGAAAACAACAAAAAAACTAAAGGATCTGAGAAATAATTAGTGTTTACTTCTGGGGAGGGGAGGAG
GTCTGGGATGGGGGTAAAAAGGATAGTCTTATCTATTATGTATATTCAGGTTTTTGTTTTTTACAAGAAGCATGTATTAGGTATTATTTGTATA
ATAAAATATAATTTTAAAAATACAAGAAATTTCTCATATAAAAATATGAAAGTAATCAGACTGCAACACTCAGTGCCTGAGACAGAGCTACAGC
TATCAGGGTGTCCAGACAGACAGAAGATTACATTTTCTTCCTTGCTCCTTGTACAGCCCCAGACCTGCATGCTTCATTGAAAAGAAAAGAAGAT
ACCTGAATTAAATCAATGTGATGCTTAGTACCCTATCAGTGCACATTTCTTTTCTATTTTTAAATTTTAAAAATAACACTTGGCCAGGCGCAGT
GGCTCACGCCTATAATCCCAGCCCTATGGGAGGCCGAGGCGGGTGGATCACCTCAGGTCAGGAGTTCGAGACCAGCCTGGCCAACATGGTGAAA
CCCCTCTGTACTAAAAATAGAAAAATATTAGCCGGGCATGGTGGTGGCCACCTATAATCCAAGCTACTCAGCTGAGGCAGAAAAATTGCTTGAA
CCCAGGAGGCAGAGGTTGCAGTGAGCTAAGATCATGTCACTGTATTCCAGCCTGGGTGACAGAGTGAGATTCCATTTAAAAAAAAAAAAAAAGA
AAAAAAAACACTTATGGCGGTATTCTCAGTCATTACAAATAAATAAAAACAATCCATATGCCCTGGAGAATTTGATTCCAGGAGTAGGTCTAGA
AGAACTTCAACTGGAGAATGGATAGAGAAATCATGGTATATTTGCAGAATATATATTATATATATAATAGATAGCATGTGAATAAATTAATTAC
AAAAACATATGACTACATCTATTATTATATAGCATGTAGATAAATTACAAAAACATGTAACTACATCTATGAATCTTAGAGCATAATATTGAGT
AAAAAATAAATAAATAAAAATTAAGGCCAGAAGATAACACATAGCACAATGTCCTTTTCATAAATAAATATATTGCTTAAGCATACCTTATATA
TAGAAGATAAAGCTTAAAAAGTAAAGAAGAGGCCGGGCGTGGTGGCTCACGCCTGTAATCCCAGCACTTTGGGAGGCCAAGGTGGGCAGATCAC
GAGGTCAGGAGACCGAGACCAGCCTGGCCCGCATGGTGAAACCTCGTCTCTACAAAAAATACAAAAATTAGCTGGGCATGGTGGCACAAGCCTG
TAATCCCAGCTACTCGGGAGTCTGAGGCAGGAGAATCGCTTGAACCAGGGAGTCGGAGGTGGCGGTGAGCCAAGATCACGCCACTGCACTCTAG
CCTGGGCGACAGAGTGAGACTCCGTCTCAAAAAAAAAAAAAAAAAAAGTAAGGAAGAGACTGATAAGCCCGATATTCAGGACGCCAGTTACCTC
TGGTGCAGGCAGACATACATCATTAGTAATGTGCTAGTTCTTAGGGTAGGTGGTAGGTTCACAGATGTTCATTTTACTTAATTAAATATATTTT
GAAAATTTATTTAAAGCTTTCTGTTGTAAAATATATCACAGAGAAAACCACATGAAACAACTATAAAGCTTAACATTACTATAAGGTGATTACT
TTTGTAACCACCACCCAGGTTAAGAAGAACTTTGTCAGCTCCCCAGAAATGCTTCACATACCCTAGCCCAATAAAACCTCCTTTGCAAATACTC
TTCATAGCACTATCTGATATGCCCTTTATTTTTACCTTTTTTAAATTAAAACAAAACTTTTTAGAGACAGGGTCTTCCTCTGTCATCCATGCTG
GAGTGCAGTGGCCCAACCACAGCTCACTGCAGCCTCCAACTCCCAGGCTCAAGTGAGTCTCCTGCCTCAATCTCCTGAGTAGCTAGGACTACAG
GCATGTACCACCCGGTCTGGCTAATTTTTTAAACATTTTTAGTGATGGGGTCTTGCTATGTTACACAGGCTGGCCTCAAACGCCTCGTCTCAAG
CAATCCTCCTGCCCCAGCCTCCCAAAGCACTGAGATCACAGGTGTGAGCCATCACTCTCAGCCTGCCCTTTATTTTTTCATGAAAGAAATTGCT
GAAGAGGACTAAAAGAAGTTTTAGTAAGCATTCAATAAATGTATGTTCTTTATAGTTTCCAAATCAGCAAATATAGACATCCTGCATTTTTAAG
GAGATTTATATATTTTATTGGACATGCTGTAATTTATTTAACCACTTCCCTGTTGGTAGACATTATTTCCATTTTCTTCTGCTAGATTAATGCT
TGAAAAAAATGTGTGCCTCCTAAAGACTGTGATGAAAGTTGCCTCTGAATAAAACTCAAACAAATCATTAATCATTAACTCTTTCCTTACTTGT
ATGCTCTTTGGATGCTCTACTGTGTTATCTATAAAATAAAGTTTGAAGTGAAAAATTAGGGTAAAACATTTTATATCATTTTTAAAGGATATAT
ACATGGATGTACTTACATATGCATGTTTAAATTTATATACCATAACATTTATTCTTTTTTTAAAAAAGTCTTATGAATTTTAACAAATGCATAG
TCCTATAACTACCACCACCACAGTGAACATCCAGGACAGTTCCATCCCTTGCCAAACAAACAAACAAACAAAAAGCTTCACCCAAGTCTTAAGT
CCAGGCACTTACTGATCTATTTTCTGTCCTTATAGTAATGACTTTTCCAGAATGTCATATAAATGGGATCATTCATTAGATATCCTTTTGTATC
TGGCTTCTTTCACTTAACATAAAGCATTTGAAATTCATTCATATTGTTGTGTGAATCAATAATTTATACCTGTTTGCTGTTGAGTATATTCTAG
TGTATGTATACTATATTTTGTTTATAAATTCCCCAATTGGGGCACATTTGGGTATTTCCCATTTTGGGTGATTACAAATAAAGTTATTATAAAT
ATTTGCATATAGGTTTTTTTGGGCAAACCTAGGTTTTCATTTCACTTGGATAAATACCTAGAAGTGGGATTGCTAGGTCACATGGTAAGTGTAT
GGTTTATTGTGAGAAACTGCCAAACCTTTCCATAGTGGCTGTACCATTTTTTCATTCCCACCAGCAAGTATATGAGAGTTCTAATTGTTCCTCA
TCCTTCCCAGCACTGGTATTCTTTTCTATTTTTTCTTTTTTTTTGAGACAGAGTTTCGCTCTGTCACCCAGGCTAAAGTGCAATGCCGTGATCT
TGGCTTACTGCAACCTCCGCCTCCCGGGTTCAGGCTATTCTCCTGCCTCAGCCTCCCAAGTAGCTGGGATTACAGGCACCCACCACCATGCCCT
TCTTTTGTTTTGTATTTTTAATAGAGACGGGGTTTTGCCATATTGACCAGGCTGGTCTTGAACTCCTGACCTCAGGTGATCCACCCACCTTGGC
CTCCCAAAATGCTGGGATTACAGGCATGAGCCACCACGCCCTGCTCTTTTCTTAAGTCATTCTAATAGGTGTGTAGAAGTGGTTCTAACTTGCA
TTTTCCTAATGACTAATGATATTAAGTATACATATATTTATATATTATTTTATAATCTTATATATAATATACATATTTTATATATTATATATAT
ATTTTATATATATATACACACACACACACATATTTTTTTCTTTTTTTGAGACAGGGTATCACTCTGTTGCCCAGATTGGAATACAGTGGTATAA
TCATAGCTCACTGCAGCCTCGATCTCTCAGGCTCAAGCGATTCTCCCACCTCAGACTCTGTAGTAACTGGGACTACAGGCCATGCCACCATGCC
TGGCTAATTTTTTTTTTTTAATTTTGTAGACATGGGGTCTCCTTGTGTTTCCCAGGCCAGTCTCAAACTCCTGGACTCAAGCCATCTTCTCGCC
TCAGCCTCCCAAAATGCTGGGATTACAGGCATAAGCCACTGCACCTGGCCTTAGTTAAGTTTTTATGTACTTATTGTATATCTTCATTGGTGAC
ATTCAAATCATTTCATCCTTTGTAATTGTATACTAAGGAATTAAACGATTGTTTAATTATTGTTTATTTTCTTATTGTAAGGTTTTAAAAATTC
TCTATTTTTTTGAGACGCAGTCTCCATCAGTCGCCCAGGCTGGAGTTCACTGTCGTGGCTCACTACAGCCTCTGCCTCCCAAGTTTAAGCGATT
CTCCAGCCTCAGCCTCTTGACTGGCTGGAATTACAGGCATGTACCACCATGCTTGGCTTATGTTTGTATTTTCAGTAGAGTCGGGGTTTCACTA
TGTTGGCCAGGCTGTCTCAAGCTTCTGACCTCAAGTGGCCCACCCACCTTGGCCTCCCAAAGTGCTGGGATTACAGGTGTGAGCCACTGCCACG
ACCTTAAGAGTTCTTTATTCTAGATACAAGTTCTTTATCAGATATTGATTTTCAAATATTCTTTTGAAGTCTGTAGCTTGTCTTTTCATTTTCT
TAACTGTGTCTTTTGCAGAGCAAAGATTTTAATTTTGATGAAGTTTGATTTATCAATGTTTTCTTTTATGGATCATGCTTTTGGTATCATATCC
AAAAACTCTTTACATAACCCAAGATGTAAAAGATTGTCTTCTATATTTTCTTCTACACATTTTGTAGTTGTATATTCTATATGTAAGGTTTATA
ATCCATTTTAATTTTTATATAAGGTGTGATGTATAGGTTAAGCTCATTTTAGTTTTACATGTAGATGTTCAGTTGTTCCAGCCCCATTTGTTAA
AAAACACCCATCCTTTCCCCATTCAATAGCCTTTGAATCTTCATCAAAATCAATTAGTCATATTTATGTGGGTCTATTTCTGGACTTTTTGTTC
CATTAACTTATGTGTATATCCTTTTGCAAATATCACACTTATATTTGTTTTTTTTCTTACATTTTTATTTCAAAATCTAAGGACATCTTATAAC
CCAGAAATATTTTTTATACCTTGTCATGTCTTAGAGGAAAGAGCCACCCCAGTCTTTTTTCATTGATGTTTTTCTTCTCTCTTCGTACTCCAGA
GGTAGATGAAAACCAGAGGGCCACAATGACCATGGTGATGCCTGAGGTCATTCTGGGGCACAGACCTCAGCCTAGGTTACTCCACTTCGCCTAT
CTTTAGATCCAAAACTACCCTGCTGACTGCTGAGATAAACAAAGGAGAATAATCAGGTTGGGGAAAGGATTTCTATGCGAAGACATGTCTCCAT
GCAGTCCTCCTACACTGAGCAGAGCATGAGTCAGGTGCTTAGAGCAGGATTTTGTCCTAAACCAGGAACTTCAGAGTTTTCTGAAGAATGTGGC
TATGTAAAGCACCCCCCCACCCCACCCTTACTTCTCAAGTACATTACGTGGCAAGTCTGAAAAAACTTACACTTCTGTTGTTAAATGTGGGGGA
TAAAATATAAACTTAGTTTCAAGAGGAAGCTATCTTGGGAGGTAATGCAAATAATTCGTTGTGTGTTTCCTGAATAAGTGACAGGTGCTGACTA
CCATTGATGCTTCATTGCAATAAAATGCAAAGCTCCCCCAAGAATTTTTGAAATGCATCAAGCTAGGTGTTCTAATCTAGCAAAAGGACCTGCA
TACATGAATTTTTCATGCTTTTGCCAAGTCTTTTGCCCTTTAGTTTAGTTAAGGGCCCCACATGAATGGAAAGCCTGTGTTGTCAGCTTAATTT
TGTAGTTGTGGAAACCTTCCAGTTTTCTCCTTTGTCTAATACCTTCAGGAGTTCAATCCTAGGTTGAAGCTTAATTTAATAACCATGTGGCATG
TAAAGTAGAAAACAAAACATCTTTTCCTTAGCATACAGCAAAAAAAAAAAAAAAACTCACTCATGGATGTAGTGTACACATGCCAGTGGATATA
TAGTCATAACTGCAGTCATTGGTAGCACAGAAATAAATGTGCATTGAAGACACAGAGATGAATTTGAATTCCGGTTCCATGAGCTACCATTCTT
TGACCTTGGGCAAGTTGCATCACTTCTTTGAGCCTCAGTTTCCCCATCTGTAAAATGTGGATAGCAACATCTACCTCGCAGAGTTGCTGGGAGG
ATCAGATGAAATGATGGGCTAGCCCAGTGTCTGACTTACTGTGTTTAAGAAATATCAACTATTACGCTACTTCCCAGTGACAGTCCAAATGCAG
ACCAGTGTTATAACTCTACTTCTCAAACATTAATGTGCATGCAAATCACCGTGACCTTCTTAAAATGCAGATCCTGATTCCCTAGGTCTGGGGA
AGGCTTGAAATTCTACATTTCTACCTAGCTTCCAGGTGAAGTTGAGGACAACACTTTGAAGGCAAGGTGGTAAAAGACGCTTGCACGTGCAAAT
GTTACTTTGTGTAACTAATGGTTCGCAATACTGTCTGCACTTTAGAAGCTCCCAGGAAAAGAAAATCTTTAATGATGCTGACCTCATCTCCAAA
ATAATGTAATTTTAACAGATCCAAAGTAGTGCCTGGGCATTAGTGTGTTTTAAAAGCCTCCCAGGTGATTCCAATGTTAGCCAAGATTGAGGCC
CTCTGCTTAGTGAATCTAATTTGCAGCTGGGCTTGAGAAAAAACCTCTTCATAGAATTGTTTGCATCAGTGTCTTGATTGCCTCTGTAACTTAC
AATAAGCAAGAATGTTTCAGGATTTCAAAAATCTATTGCATTGCCTAAACCTCTTATTTTGTATGGAGTAATCAAGCTCAAAGTTTGCATGTCT
TAGAAACTTTACTTGGGGCAAAATTAGACCAAGTAACAATTAATCTTCTAGGTATTCTGAGCTATTCAGACATATGATTCATGTTTGCTAATTG
CTCTTTTCTCTTGTAAATATTAGCTGAAAAATGTCACCTGTCTGACAAGTAGCATATTTTATGCCTATCACTCCTGGCACGCATTCTTACAAGG
CAGACAGGAAAAATAGGAAGAAAATGGACTTTTATCAAAGGCCCAGGCAGTAAAGAGGGGAGTTCTGCTGTAAGCTAAGGGGAGTTCCAGAGGA
AGTTATAGGCGTTCCCTTTCTTATGACAAGAAAGCATAGTGCAGTAAATAAATTTGCTAAATAGATTCAACAGTCTCTACCCAAAGTCATCTAT
TTAATTCTTGTTGTTATGCAGACTCAGCAACTAACCTTCCTTGTAAGCCCCATTTTCTTCCCTGTTTCCTGTTTATCAAATGTAATTAAACAAG
AGAAGTATTATAGAAGAGTAAAAGTAGTAGGTAATTCTTGAACTTGGCATATGATTACTACATATTTGATGAATAGTTGAATATTATTCTTCAA
GGACAGATTGGATTTGGTATCAGGTGGCTCTGCATTAAGTTATAAGGGACTTAATAACTCAAGTATTTAAGGACGGCTTCCATCATAAAGGGAT
CTGCCCTTAAGAGGGTCCCATTATGGAGATTCTGAGGTGAGAGCTATTCCAAGTGTGCAGTGGATTAAAATAAAAGAATCATACAGGAAATCTC
TTTTTACATGCCTTATTCCAGGGTCTTTGCAACCTGGCACAGCAAGTGCAGATATGATTAGCATTGTTTTACACATGTACACTCACCTTATAGC
CCTGCCCCTGTGCCCCTCCTGCACAAAAGAATGCTGGGCACACGTGAACTCCTCTCTGTAGAAAGGCACATTAATGTTCTAGCCATGGTTAAAA
CAGGGATAGAGGCAAGCCAAAAATGTCGGTCATTTGAAATAAATCTCAAGTTTGTGCATATCACTATCAAGTGTGCTGTGTGGCAATTAAGAAT
GCCAATTTGTGTGATCACAGGCAAGTTGCAGTTTGATGAAAGGAAAGCAGAGGTGAATATATAACCAGGGTCATCCTTTCTTTCTCCCTCTCTC
TCTTTCTGTCATTTATTTGCCAAGCTCTTAACTAGAACTTGCTATGTGCTAGGTACTGGATATATCAAAGCAAACTCAGCCTGGTCTTTGCCTT
CAAAGATTTGCAGGATAGTGGGAAGAAAAACTTGAATCAGAGGACATCTGCAGTGGGAATCATTCAAGCAGCAGAAAACCCAAAAGTTACTTAT
ACTGTGAAATCTGATCAGAGAATGGACTGTCCTGGTTAGTAAAATATCCTGGAGGATAAAGATTGGCCATGCATTCCACATATGAATTACCACT
TTCCCAAGAATTAAAACATGGTACGAAAGAAAGGAACGAACATTTGCTGAGTGCCTACTGCCTGCCAGATCTAGGCACTTCACATGTAACACCT
CGTCCAGCCTCCACCACAATACTGAGAGGTAGAGTTCAACCTCATTTTATAGATAAAGAGGCTGAAGCTCATAAAGATTAAAGGACGGACTCAC
AGTCAAAGGACTCCTAATCCTGAGTCAGCATTTGAACCCAGGCTCACCTGTCACTATATCCTATGCTCTTCCGTATCACATTTTATACTGAAAC
AACTTCTGGAATAGCTAATGCTTAGAAGCAGCTCCCAAATATTTGTTGAATGAATGATTTGATTGAATGAATGAATAAATGAATGAAGCAAGCT
CTACTGAACATAATTTGATCTAATCTTCTGTGATTATTCAGAAACTACTTCAAGATTTTCCTATACCTCCATCATAATGAATACCCATTCATTA
ATGATGGAAGCAGCCTAATTTTGTCATTTTTCACACTTTATTGATGTAACACTACCTTTACTAGTTTGGCCACTCCTTATGCTTTTTTTATAGA
ACTATTTAGATCAATTCAACTTTTAAAAAATAAAGCCACATACCCCTGTGGTAGATGAAAAACAAGTATCATTTGCACTGGTAAATAGAGAATA
GGAAGAAAAATAAATGCAGTGAAAATAAAGCAGTGTTATCAAATCCTACCCAGATACTGTTATCTACCCGGAAGCTTCCTGTTTGATTAAAAGG
AAAAATAGCCAGTGTTAGAGGTGTGGAAGTCTAGTTGAAATTATATGCAATTGAAGGATTAAAATAGAATTGAAAAGGGAATAAATTCCTCTCT
GAATAATTTAACTCCCTTTAGGCTTTGATTCTGCCTCATCTAAAATCATCTTACATACTTCTAGTGGCGTGTCCCTCACATTTTGGTAAACTCT
GAGTGGAAACTACGGATTTTGTAGTCAAACATCTGACTGGCTCTGGATTTTAACTTTACTAACATATGTCCTGAAGTGAACTGCTTGGAGCCAG
TTTCCTCAACTATATAACCATAATGACAAAACAGGTTTCACGGTCATGTTTTCAGTATTAAACAATATAGTTAAAAGTACCTAGCACAGTGCCT
GGCATAGAACATACTAGATATACATTAAGTATCAGTTCCATTTTTCCTTTCCCTTTATTGTCAGAAAATAGAAAGCATCTACAGTGGGCTTGTA
TGATGTGGTGGTTAGAAATACCTGATCTGATTCTGGCTGTGCAATCTTGGGCAAGTTACTTAACCTTTTTGTGCCTCATTTTTGTTTTCTGAAA
AAAAGGATAGAATATTATTACCTACCTTGCTGGCTTCTGGGAAGAAGCTCAGTGAGACGATGTGTTAGCAGAGTGTCTGACAATTGTAAGAATT
CAACAAGTAATAGTTATTATTACCATCACTGGTGAGAGGAAGTGATACCTGGCACAAAAATATATGGATTAATCAATATGGATTGAGGGAAACA
AACCTGGAGAATAGGATGTGAAGGTATTTAAGTAACATGAGCTCAGACCTTGATGGTAGGGAAGTCGAAAGGAAGCATTTTGTTCTTATATGAC
AGATGACCTGGAATGACTGCAGGGCTTGGGGGGTCAGGGACTGGAGGTGGGAGAGGCCTCTGAGAGCAAGCAGTGCTGTCCACCAGAAGCTCTT
GCTGGGGTGCCCAGAGAGGAGCAAAGGGCAGTCAGCTGCACAGGAGGGAATGTTTGGAGGAGAGAGCCACCTCAGATCAGCGGGTCAAGAATCC
CACTCTTGCCCAGATGGATGGGGCAAAGGAGAAAAAGGATTCGCCACGGGAATGTCCAGATAAGACAGGTGCCTTTTGGAAAATGGGGGTGAGA
TGGGTCTCAGGTTACACTTCGTAAGAACTGGAATGTAAAGTAAAGGCAGACAATGACAAAATATCTTGTTTTCTTTTCAGCTTTATTCACAGTG
ACAGTCCCTAAGGAACTGTACATAATAGAGCATGGCAGCAATGTGACCCTGGAATGCAACTTTGACACTGGAAGTCATGTGAACCTTGGAGCAA
TAACAGCCAGTTTGCAAAAGGTGGAAAATGATACATCCCCACACCGTGAAAGAGCCACTTTGCTGGAGGAGCAGCTGCCCCTAGGGAAGGCCTC
GTTCCACATACCTCAAGTCCAAGTGAGGGACGAAGGACAGTACCAATGCATAATCATCTATGGGGTCGCCTGGGACTACAAGTACCTGACTCTG
AAAGTCAAAGGTGAGTGGTGTCAAGGACTAGAATCCATGGAAGCATCTCTCCAACAGAGGATCTGCAAGTCACAGAAACCCATTAAAGGTAGCT
CAAGCAAAAACAAGCAGGCTGCTTTTAAGGAGACAGCTATTTCAGAGAAAATGAAAGCATCTGCTCGGAAATAATTTTTGACATCTGAGTACAA
AGCAGCCGAAGTACAAGTGAAAGGGGGTAGGACCTATAGGAATAAAATGGGACTGGAGGAAGCCAGGAAAATTAGTCCCTGAAATGTGGGAGGG
TATGAAAAATAAGCTTTGCCTAATTCACAATTCTCCCATGGAACATCCCTGACTTGATTATTAAGATACTCTTTTTCAATAGTTTATACCCTGA
ATCCAGAGTTTTTAAAACCATGGTTTGCCGCCCATTCATGGATTAAAATATCAATTTAGTGAGTAGCAACCAGATGCACGTTTCCCGCCCTTTA
AAAAATAATGTATAGAAGAGAATAGACAGAGTAGATCAGACGACATCACAGAGTAGGACTGAGTACTGTAAAACTAATTTCTGAGGGACGTGTG
TGTGTGTGTGCGTGTTGGGTCATGGTATAAATTTTTTTTTTCTTACTTTGGATCATAAAAAGTTACAAGTTTGGAAAACACTGCTCAAATGCAA
GCCCTATTTATTGCAGATGAAGTAGCTGAGATCCAGAGAAGGGAAGTGGGTAGCCCAAGAGAGTCCCATGAAGTCTATAGCTGTCCCTATTCCT
CTGGATTCAGGGATCTCTCCACTCCAGCACAATTGAAAATCTAAATATAAAGAGAATCTTCACACTCTTGTTTGTTCTAGAAAAGGTGATTTGA
GGAAAGACATATAACAACTATAAAAAATAGATTTTGCTTGTTCATTGGCTTATGGTCTCCAGGCTTGAATGCTCTGAGATAAATGATGCCAATA
TTTCTCTGGCCTCTTCCCCTCCCACGCATTGGACCTCAGATGGTCTGTACTGTCTTCTAGAGGGTTTGTGGGTTTTGGCCCCAAAAAACCATTA
ACCTTGGCAGAAAGTGTGTGACTTTATGATCTGGTACAAAGAAGGACAAACTAGAGGGACTGGACATGAGGATGAATATTGTGTTCGCCCTTAT
CCTTGGGCAGGTTATTTTGCCTTTCATAACCTCACATTTTGCTTATTTGTAGAAGACGAACAATAGTAGTACCAAACACACAGGATTGTTGTCA
ACATTGAGAGAGATAATGAACATAAAGAACTGAGCATGGTACCCGGCATATAATAAGTGTCCAGAGACTTGGTTAAAAGACCCTCAGTTGTTAC
AGGGGCAGTGACCTCCTCACACCTCAACCATCAATGAGTCACCAGGAAAGCCATTAGCCTAGATGTAACTGTTTTCTATCTTTATTGCATTTCC
TACATCCAGGCAGCAGCTGGGAGGAACTCTAGAACACTGAAGTTTGTCTGAGTTCCCTTAATGTAAGGCTGTACATTCTCAGGATGCCTTGATG
TACTCGAATATCTGCAACCCTAAATCACCACCTCTGTTTTTATTGATCTCTATCTGAATGCTGTATTAATGGGCCAGGCCTTCTGCCCATTCTC
TCAAACTGAGAACTGTCTCTCATTCCTGGGGAGGCACCCTGCCTACTCCTTACCTAGATCAGGGATTTCTCAGTTGTGGAGAGATTTGTTCCTT
ATAGTGTTGGTCATCAAACTGGGATATTTGGGGATTACAAAGACTTTTCAAGGGATGTATGGGCACAGGCAGTTTTAGGAAGTGAGTTCCTAGA
TCCTCATCTTCCCCAAATACTCGTTCCCAAAATTGACGAGCCTGACAATGTGCATGCCAGGCAAGGCTCTTGGGGTTCCCCTAAAACACTTCCT
CTTTTAAGCCTACCACTCACTCATCATGAATATAGTCCATTGTCCCAGGGTGTAAAACCCTCTATAGTGTTAAATAAAAGAATGATTGGGAACA
TTGACACCTGATGGAACTGTTATGACTAAAAACCCTTTTGCAAATAATGTGGTATCTAATTTTCTGCTTTCAACAAAATTGAAGGAGGCCCTTA
TAAAGTTAATAACTGATAATCAAAAATGAGTAATTTTTGCCATGTAAATCAGGTCAAAGAATGAAATGGCATTGCTGTAACGAAACTGCTTCCA
TTCCCATTGATTTACTCATACGAACAAGATTCCTTAGCCTTTATAAGCTACAAAAAAATGAAAAATAGAAATAGAATTGAGGCTGAATTCTATT
ATATAAAATCATTCCAACCATGTCATATGGTTCTTCGGATTCATGAATAATTTGGAAAAGAGAGCCATATCCATCTTATTAAGGGACACATTCC
CAATAAATTTTCATCTTTCATGTTTAATAATTATCAATATTCATAACATTTTACATTTTGATCAAATATGTGTTAATAATAATAGAAATAAATG
TCCAACAACGATAGACTGGATTAAGAAAATGTGGCACGTATACACCATAGAATACTATGCAGCCATAAAAAATGATGAGTTCATGTCCTTCGTA
GGGACATGGATGAAGCTGGAAACCATCATTCTCAGCAAACTATCCCAAGGACAAAAAACCAAACACCGCATGTTCTCACTCATAGGTGGGAATT
GAACAATGAGAACACATGGACACAGGGAGGGGAACATCACACACTGGGGACTGTTGTGTGGTCGGGGGAGGGGAGAGGGATAGCATTAGGAGAT
ATACCTAATGTTTAATGACAAGTTAATGGGTGCAGCACACCAATATGGCACATGTATACATATGTAACAAACCTGCACGTTGTGCACATGTACC
CTAAAACTTACTGTTAAAAAAAAAAATTAGATCTTAATGCAGAACACCCTGAACATTTAAAGCTTCATAGTCACAAGAGAAAAGTTTTCATTTC
AATAGCTATAAATATTTTGTTGTTGTAAAGACATATAACGATAATCAATACAAAATCTGTCAAACAAAAATATGTTACATTAAGATAAAATTCT
GTAGGGAAGGTGAAATTGGAAGTGAGTTTCAATGAATGAAAAGAAACAATTTAGACAGAGAAGAATTTTTTCATTTAATAATTTATTTATTTTT
ACTTAGAAGGAATTGAATAGATTAGGTTCCTTACCCAAAAAGCCTCTGTTATTTGTCTTATTTATTTATTCTCTTTTTTCCACATTCTCCAGTC
TCATTCCCCTTTTTTAACACAGGAAATTATTCCAGCATGTTTCATACATATTCTTTTGTTTGTAAGAGCTTATTTAAAATATGTAATATTGTTT
TAGATGCATATATTTTTTTTCTTGTGGAAACTATATTGTACTATATATATATATTTTAGAAATGGACACATTAGGCCGGGCGCGGTGGCTCACG
ACCGTAATCCCAGCACTTTGGGAGGCCAAGGCGGGTGGATCATGAGGTCAGGAGATCGAGACCATCCTGGCTAACATGGTGAAACCCTGTCTCT
ACTAAAAAATACAACAAAAATTAGCCGGGCGCGGTGGCGGGCGCCTGTAGTCCCAGCTACTGGGGAGGCTGAGGCAGGAGAATGGCGTTAAGCC
GGGAGGCGGAGCTTGTAGTGAGCCGAGATCGCTCCACTGCACTCCAGCCTGGGCGACAGAGCGAGACTCTGTCTCAAAAAAAGAAAAAAAAAAA
AAAGAAATTGACACATTAAGTTTATTGTGAAAACATGATTACAAGCAATAACAGATATAACAGCAGAAAAAATTGTGAAGTTATGTGCACCTTC
TTTTGAGATCATTTTTTTGGGTATATGTGAATGTATTTTATCTTTTTTTAATTGACAATAATTGTACTTATTTATGGGATACATAGTGATGTTT
CAATATATACAATGTATAGTGATCAGATCGGGGTAATTAGCATATTTATCATCTCAAATATTTATTATTTCTTTGTGTTGAGAACATTCAATGT
ACCCCTTCTGGCTATTTGAAGCTATATACTGTTGTTAAATATTTATATGTATTTTTCAAGGATAATGTTGTATATAAAAGATAACTTCTTTTAT
ATTTAGTCACCTTTGGATACATATGTAAGAGTAAAATAATTGTTTTACTAAAAAAATTCATAAGATGCTGAACATTATATGTTTTTCCAGTATA
GAACTTATGAAAAATTATAAGAATCAACTTAATAGGTGAGAAGAGCATACTGCATCATACTTATTTTAGGAGGAATACAAGCAAAAAAAAAAGT
TGGACCATCACCTAAGAGTAGGCTACCCTGATCCAGGGTCTCTAAAAGAAGCAAGATAGATCTGAGGCCCCAATGAGGGTTCCCATGAATGAAG
TAGGACTAGAAGAAGGACATGTTTTTATCTTAAAGTGCCTGATCCATGGCATAAAGCTTGAAAATGGGTTTGTTTATAGCACCTTCGTTGATGT
CTGGGCAGAAATTCTTATCAAATATAATGAAAAGAGTTAATTTCAAAAGGACAGAACAAATTATGAGGTGGAATGACCGGAGATTCTGGAAGAG
ACAGTGATAAAGGACTGTGGTTTCTGAAAAGCCAGGTGGCACCTACATTTCTGGGGGCAATCCATCTGAGGAAGGGGCGGGAGCATGGCCAAAG
AACAGAGAGGCCCCAGGAGCCCAGGGGTCTCTCAGAGGAGCTGGGGTTGAGAACTGGTCTGTGCAAAGGTTTCCAGACAGGGTTGCAGAGGAGG
CACTAAAAACAAGAACACCAAGAGCCAAACCATCCTGAAGGCCTGGGCAGGGCAGGGGGATGGGAAGAGCCAACACCAGGAATGGGCTGAAGCT
GACAGAAGTACTTCAAACCAGAAACAATGAATTTAAACTATGCCCTACCACAAATAGACTTAACAGATATTTACAGAACATTCTACCCAACAAT
CGCAGAATATACATTCTATTCATCAGCATATGGAACATTCTCCAAGATAGACTATATAGTAGGCCACAAAACAAGTCTCCATAAATTTAAGAAA
ATCAAAATTATATCAAGTACTCTCTTAGATCACAGTGGAGTAAAACTGGAAATAAACTCCAAAAGGAACCTTCAAAACCATGCAATTACATGGA
AGTTAAATAATGAGTGATCATTGGGTGAACAATGAAATCAAGATAGAAATTTAAAAACTCTTTGAACTGAACGATAATAGTGACACAACCTATC
AAAACTCTGGGATACAGCAAAAGCAGTGCTAAGAGAAACATTCATAGCATTAAATGCCTACATCAAAAAGTCTGAAAGAGCACAAATAGGCAAT
CTAAGGTCACACCTCCTGGAGCTGGAGAAACAAGAACAATCCAAACCCAAACCCAGCAGAAGAAAAGAAATTACAAAGATCAGGGCAGAACTAA
ATGAAATTGAAACAAAAAAAATACAAAAGAGAAATGAAACAAAAATCTGGTTATTTGAAAAGATAAATAAAATTGATAACCAAGAAAAGAAGAG
AGAAGATCCAAATAATCTCAATTAGAAATGAAATGGAGATATTACTACTCATATCACAGAAATACAAAAGATTATTCAAGGCCACTATGAACAC
CTTTACACGTACAAACTAGAAAAGCTAGAGGAGATGGATACATTCCTGGAAATATACAACCCTTCGATTAAACCAGAAAGATATAGGAACTATA
AACAGATCAATAACAAGCAGCAAGATTGAATGGTAACTTTTAAAATTGTCAACAAAAAAAAATCCAGGACCAGACAGATTCATAGCTGAATTCT
ATCAGACATTCAAAGAAGAATTGGTACCAATTCTATTGACACTATTCCATGGGATAGAGAAAGAGGGAATCCTCCCTAAATCATTCTATGAAGC
CAGTAGCACCCTAATACAAAAACCAGGGAAGGACATAACAAAAAAAGAAAACTACAGGCCAGTATCCCTGATGAACATAGATGTAAAAATCCTC
AACAAAATACTAGCTAACCAAATCCAACAGCATATCAAAAAGATAATCCACCATGATCAAGTGGGTTTTACACCAGGGATGCAGGGATGGTTTA
ACATCCACAAGTTAATAAATGTGATACACCACACAAACAGAATTAAAAACAAAAATCACATGATCATCTCAATAGATTCAGAAAAAGCATTTGA
CAAAATCTAACATCCTTTATGATTAAAACCCTCAGCAAAATTAGCACAGAAGGAACATATCTTAAGGTAATAAAAGCTAACTACGACAAACCCA
CAGCCAACATTATGCTGAATGGGGAAAAGTTGAAAGCATTTTCCCTGAGAATTGGAACAAGACAAGACACTTTCACCACTTTTATTCAGCATAG
TACTGGAAGTCCTGGCCAGAGCACTCAAAGAGAAAGAAATAAAGGGCATCCAAATTGGTAAAGAGGAAGTCAAACTGTTGCTGTTTGCTGATGA
TATGATCATATACCTAGAAAACCCTAAGGATTCATCTTGAAAGCTCCTAGAACTGGTAAACAAATTCAGCAAAGTTTCAGGATACAAAATTAAT
GTACACAAATCAGTAGCTCTGCTATACACCAACATCAACCAATAGCTGCAAAAATAAAATAAAATACTTAGGAATATACCTAACGAAGGACATG
AAAGACCTCTACAAGGAAAACTACAAATCACTACTGAAAGAAATCACAGATGACACAAACAAATGGAAACACATCCCATGTTCATGAATAGGTA
CAATCAATATTGTGAAAATGACCATACTTTGTAGATTGCAAAAGCAATCTACAAAGTCAATGCAATCCCCATCAAAATACTACCATCATTCTTC
ACAAAAGTAGAAAAAGAAATCCTAAAATTCATATGGAACCAAAAAAGAGCCCACATAGCCAAAGCAAGGAAGACTAAGCAAAAAGAACAAACCT
GGAGGCATCACATTGCCTAACTTCAAACCATACTATAACACCATAGTCACCAAAACAGCATAATACTAGTATAAAAATAGGCACATAGACCAAT
GGAACAAAACGGAGAACCTAGGAATAAAGCCAAATACTTATAGCCAACTGATCTTTGACAAAGCAAACAAAAACATAAAGTGGGGAAAGTACAT
CCTATTCAACAAACGGTGCTGGGATAATTGGCTAGCCACATGGAGAAGAATAAACTGGATCCTCATATCTTACCTTATAAAAAATCAACTCAAG
ATGGATCAAAGACTTAAATCTAAGACCTGAAACCATAACGATTCTAGAAGATAACATCGAAAAAGCCCTTCTAGACATTAGCTTAGGCAAAGAC
TTCGTGACCAAGAACCCAAAAGCAAATGCAACAAAGACAAAGGTAAATAGATAAATAGGGACTTAATTAAACTAAAAAGCTTCTGCACAGCAAA
AGAAATAATCAGCAGAGTTAACAGACAACCCACAGAGTGGGAGAAAATCTTCACAATCTATACATCTGACAAAGGACTAATATCCAGAATCTAC
AAGGTATGCAAATAAATCAGCAAGAAAAAAACAAACGATCCCATCAAAAAATGGGCTAAGGACATGAATAGACAATTCTCACAAGGAGATACAC
AAATGACCAACGAGCATATGGAAAAATGCTCAACATCACTAATTCTCAGGGAAATGCAAATCAAAACCACACTGCGATACCACCTTACTCCTGC
AAGAATGGCCATAACAAAAAAATAATAGATGTTGGCATGGATGTGGTGAAAAGAAACACTCTTACACTGTTGGTGGGAATGTAAACTAGTACAA
CCACTATGGAAAACAGTGTGGAGATTCCTTTAAAAACTAAAAGTAGATCTACCATTTGATCCAGCAATCCCACTACTAGGTATTTACCCAAAGG
AAAAGAAGTCATTATACAAAAAAAAGATACTTGCACACGCATTTTTATAGCAGCACAATTTGCAATTGCAAATATATGGAACCAGCCCAAATGC
CCATCAATCAGTGAGTGGATAAAGAAAATGTGGTATATATATTATACATACATATATATATGCATATTATATATATACACCATGGAATACTACT
TAGCTATGAAAAGGAATGAAATCATAGCATTTGCAGCATCCTGGATGGAATTAGAGACTATTATTCTAAGTGAAGTAACTCAGGAATGTAAAAC
CAAACATTGTATGCTCTCACTCATAAGTGCGAGCTAAGCTATGAGGATGGAAAGGCATAAGAATGATACAATGGACTTTCAGGACTTAGGGGAA
AGGATGGTTGGGGGTGAGAGATAAAAAACTCCACATTTGGGCCAGGCGTGGTTGCTCACACCTGTAATCCCAGCACTTTGGGAGGCCGAGGTGG
GCAGATCACGAGGTCAGGAGATCAAGATCATCCTGGCTAACACGGTGAAACCCCGTCTCTACTAAAAATACAAAAAATTAGCCGGGCGTGGTGG
CAGGCACCTGCAGTCGCAGCTACTCGGGAGGCTGAGGCAGGAGAATGGCAGGAACCTGGGAGGCGGATTTTGCAATGAGCAGAGGTCGCACCAC
TGCACTCCAGCCTGGGCGACAGAGCGAGACTCCGTCTCAAAAAAAAAAAAAAAAAAAAAATGGCCTTCACCCTAAGAAGGAAGAAACCAAGGGC
AGAGGAAGGAAACAATCAAAGAGAAGCAAGGCAGCAGTGAAAAGACAACTGTACGTTTAAGCCTCTAAGGCCCAAGGAAAGCATTTGCTAAAAG
AGCCAAGTCTCAGTGTCTTTTTTTCCCTTGGGGAAACAGAAGGGTGTAAGAAAATCGGAGTCAAGAAAATGTCCTGAGCTCCGCCTGCAGTAGC
GGGAAGCCTAGGAGTAAAATAACACGTTTTTTAAAAATAAAGAAAGAAATTATTTTAAAAAAGAAAACAATGTCCTGAATTTCAATGGGGTGGG
GGGGAGGGGAAGCTCCTGGGGATTACAAACTCGTTAACCTGCTATTCCTTTTCAGGCAATTTAACAGATGAATTGAAAGCAGTCCTAAAAGATT
TAACTGATTACTAGAAACTTGCATAGATCCTCTAAGACCAAGGCATGTAAAACTACATTTTTCCATTTATTTACTGTCATTATGACTATAAAAC
AGATACTACCATGTATACATTTCATCTTTATTTCAGAAATTACAGCATTGCTTGTAACATATCTGTCAGACAGCCAGAACAAACCAGGGAAACA
GAAACCATTTAAACCTGTGGAAATCTAAAGCTGAAACTAGTAATTCTTTTAATGAGGGAGACTGAGAAGCCAAACAGAGAACAGGGAAGCAATT
CAGAAATTAGCAATAGCAGAAAGCTGCTCCTACCACTAGGCTATAGTGAAAAGGGGGAAGGCAGGGTTAATGGAGCCCAGGGATTAAAGTCACT
TGACAGACACTGGAACCCAGTGGGCCTGTCCATTGGTATCTGGAGCCAGGGAAGAGATACATCTTCTGCTGGAGAGGCCACAAAAGTATAGAGC
ATAGGGGAGAAATACCTTCCCTTCTCCCTCCAGTCCACCCTCTAGTGTTCCTGTCTTGCTTCCTATTGGCTGTCACGTTCCTAGGCTATACAGG
CCACAGGAGTCTGCCTTCCTGCAACACAGAGCAGAGAAGAAAAAGTGAGAGATGGATCTGAAGGCAAACAGACCCAGGACACACAAAGCACACA
CACCTTGTGTATTGGGTGGTAAAAGGTGGGATAATAATACAGTTGACTAGATCTATAACAGACTGAAAAGCTAGTAGTTGTTGAGTCAGGGACA
ACGAAGGCTGGCTGATGGCCTCAGTCAACTTGGAGACAATCTCCAAGTGCTAAGGGGTCTGTTATTCAGGTTGTAAGGATATAGCCAACCTAAC
TGAGCAGCAGATCATATATTTAGCAAAACTGATCTTATGACCTTAGGTTACATTAAAACCCTGGATATTTGTGCTGCTTAGATAATATCTAGAA
TATAGATTGTGTTACATATCCTTTTTAAAGAACGTGGCAAAACTAAACTATAACCAAAGACTACTATCTGGAAATGGGTCTGAAGACTGTATCT
TGTGAGATATAGTTAAAGGAGGTAGGCATGTTTTACTTAGGGTTGAAAAGACTAGATGGAGTCTTCCCAGTTATATTCAAATATCAGAAGGGTT
ACCTGCAATAATTGCTAACACTAATTGAGTGTTTACTACGTTCTAGACATCTAACTAATTATTTAACACCCTTGTTAAGGATAAGTATTGCTGT
ATCCATTTCCTAAACAAGAAAACTGAGGGTTTAAGAGGTCATATAACTAGCCCTTGGCTAAAGCCTACTCAAACTGCTCAGATTCTGAACTCAT
GTTTTTAACTACGGTGCTTACCATATGTTGTGTTACTAAACTAGACAAATACCAATGGGTAGAAGCTACAAAGAGGCACAATTTTGCTTGGCAT
GAAGAAAATCTTTCCAATGGAAGTTTAACTTGCTTATTGTATAATCATGGCAAAGACTGCTTCTAGAGGAAGTAGGAGTTTCCATTCATAGATG
TTTTCAGGCAAAAGTTATTTAGCAGTTTACTAATATCAGAAGAAAAATATTACTAAAGATAACAAGGGACACTTCATAATGATAAAAGAGTGAG
TTCTAAAGGAACACATAACAATCCTAAATGTGTGTCCATTTAACAGTATAGCTTCAAAATATTAAACAACAATTGGCAGACTTAGGAGAACCAG
TCAAATCCACAATTACATTTGAGATTTCAACTCATCTCTATTACAACTGATAGAACAAACAATTAAGAAAATAGAAAATCTGAACAACACAATT
AGCCATCTTGACCTAATAGACATATGATATATAGAACATACACCCAGCAACTACAGAACACACATTGTTTTCAAGCGTATGTGGAACAATCATC
AAAGCAATTATCTAGGAGAATTGCTGTAGAAGGTGTTTTTCATTGAGCAATAAGTCAGATGACTTGAATTTTTGGAGTACTTCCGCCTCTTAAA
TTCTAAAATTTACTCATCTTTTCCTAAGAATATCACCCTCAGTCCAGCTTCCCCACTGTGGGAAGCGATTCCTTTTCCACCACCTTCCAAATAA
GAAAGAGGCTGTGGAGACATTTCTGATACTGCAAAAATGTAGGATGTGGGCTCCTGTCCCTTCCCCAAACCTTGCTGATTCTTGTAGTGGAAGA
GCTCTCTCTCTGTCTCCCTATCTGCTCCCTCATGTATCCCAAGTCAAAGCCTCAGCCAAGCTCCCAATTCCTGGCATCCCTACTTCTGAAATCA
CTTCTCAACCAGGACACCAAGATCAGTTTCTTGGTCCAAGCCACCTGCAATAGTGGGCTGGACCCTCACCCACAGTACTCTGCTCCTGTTCTTG
TTCTACTTCATTCCCCAAAAGACTTGAGAGCTTACAGAAATAATCTACAGTATGATCATATAAAATGATTACAAAAGACATTGAACCAAGGGAA
AATAAGTGTAGATAAATAATACAAAATGAGAGATAAAGTGAGTACTCGAAATACACACTTCGGGTACTGTTTGATTATGAAAGATAGGCCAAAG
TTTTGGCTTCAAGGAGATTGGTGAGTCTAACAGGTCAAGTCCTTCAGTAGGCTGCTTCAGTGTCTGAATTGCCTTATCTTCTACTACTGCTGGG
TGCTCCTAAGAACCTCAGAGCATATTCCTCTATCTGCTTATGTTGCACCATCCCTAGACATCTGCAATTACACAGAGACACATCCTCTTATGGT
GATTCCAATTAAGAATGCATTACAAAATAGTAATATGAATTTTACCCACCTCATAGGGTCTGCATATCATTTAAATGACATAATGTATTATATG
TATTATGTATATTTATACATTAGGGGGAAAGATGTCAGAGCTTTTTGGGACATACCAGGAGGCCCAGCTGGGTGTGCTGACCACATGGAGACGA
GAACTTCAAGGGAGAGCCAAAGTGCACGCAGGTTCTGGTGGATGTTTCATTGTGCAGTGACCCTGCTCTCTAGTGAACCCAGATCTAGTGGGTG
TTGTTTGCTATAATGAATCAAATTGTCAACTCTTAACATTGAGACCCACAACACAGGAGGGGAACAGAGGGGCAAAAATTGTGAAGAACGGCAT
GAACATTCCTTCTCTTCCATAGGATGTAAAAGGACGTGGCACAAAGCTCAACACATAGAAAACAATAAGTAAATCATACACATCAGATGGTTTT
CACATGGTTGTTACCATTATTTTAATGTGAAATTGTTTTTCACAAAGTTTGTACCTATTTATACTTCCAACAATTTAGGAGAAAAAAGTTGTAT
CACACCTCTGCCTTTACTTCAAGACTACACTTACTTCAAAAATTGTATTGAAATTTAATTTCTTAAAAGAATATTTCCTACCGAATGGAAGCCA
GTGCTCATCCCCTCTTCCTCTCACCAAATATCAACAGGTGACTTTCCACACTGCCTGGTAACTGCATATGGTATTGATATTTGTTTATTTTTAG
CTATTATCTAAGACCACTGGAAGGTTTGTTCTTGTGTCTTCTAGTCTCTTGGAATATCATGCCTATTTTACTTATTTTTAAGTTTTTTATAAGG
TTGTAATGTTTTATTTTAATTTTAAAATTTATTTTTAACTGGCAATAATGTATATTTATGGGGTACAATATGATGTTTTGATCTATGTATAGAT
TATAGAAAGAGTCAATCAAGCCGGGCACAGTGGCTCATGCCTGTAATCCCAGCACTTTGGGAGGCCAAGGCAGGTGGATCACCTGAGGTCAGGA
GTTCAAAACCAGGCTGACCAACATGGTGAAACCCCATCTCTACTGAAAATAGAAAAAATTAGCCAGGCATGGTGGCAGGTGCCTGCAATCCCAG
CTACTCAGGAAGCTGAGGCAGGAGAATTGCTTGAACCTGGGAGGTGGAGGTTGCAGTGAGCCGAGATCACGCTGTTGCACTCCAGCCTGGGTGA
CGAGCGAAACTCTGTCTCAAAAAAAAAAAAAAAAAAAAAGAATAAATCAAACTAGTTAACATATCACATCACCAAGTTGTTTTTTTGTGGTCTT
TATTTTTTTCTCTTCTTAATCTCAAAACAGAGAATGTAAAAAATTCATGCTTTTAGCAAATTTGAATACAGAATGCATTAACTGTGGTCATCAC
ACATGGCAATAGATCACTAAAACATTTCCTCTACTCTGAGACTGAGCAACATCTTCCACATCCCCAGCCTCTGATAACCACCTTTCTACTCTGT
TTCTATGGGATCTACTTTTTTAGATTTCACAAATAAATGAGATCATACATTATCTGTCTTTTTGTGCCTGGCTTACTTAACTTAGCATAATACC
CTTCAGTTCCATCCATGTTGTCATGAATGACAGATTTTACTTCTTTCTAAGGGCTGTAAGTATTCCATTGTGTATATATACCACATTTTCTTTA
TTCATTCTCTGTTGATGGACATTTAGTTTGGTTCCACATCTTGGTTATTGTGAATCATGCTGAAATGAATAAAGGAATACAGATATCTCTTCAA
CATACTAAATTCAATTCCTTTGGATATATACCCAGAAGTAGGACTGCTGGATCATGTGGTCATTCTATTTTTAGTTTTTTGAGGACCTCTATAC
TATTTTCCAAAATGACTATACTTAGATCTCAGCTTTCTTGAGGCAGGGAGCCATATCTGTTTAATTCACTCAGCATATACTGCAAAGAAGCAGG
TGTGTGTATGAACTGTGTGTGTTTGTGTGTATGTATCTGCATGTGTTAGGGAGAAGTGCAGAATAAATATACCCAACTCTTTACTATGTATAGA
CATTATCTAGGTCTTTATTTTTTTTCTCTTCTTAATCTCAAAGAAAACAGAGGAAAGGAGGAAGTAAAAAGTAAATTTTTGCCTGAAGATGTTT
GGAAAAAATACCAAATAAAGTGAGATAGTGGGTAATCTAGTGATTTTTATTTTTCCGTCCTCTTTCTGGCCTCCAATTGTGAAATAATTTATAG
CACTGTAAGAAAGAAGCCACAAATTGTGGTAGCTTGGACCACTGTTGAGGTCTGGAATCAGAATTCCAATGTTCAAATATTGGTCCTACCAGTT
ATAAGCTCTATGACTATGAACAAAACACTCAACTTCTCTATCTTCAGTAACCGTCCACAAGAATTGGGAATATCAACAATGCCATCTTTATTGC
ATTGTAAGAATTAAATAATTCAATATATGTGGAAAATAGCATGATATCAGGGATATACAATGTGTTTACAAATGATAACCATTATTACTTAATA
TTGATAGTGATAATTTTATTCATTTCACATGGCATGAAGTACCAAGCTCTATAGGAATCAGAAAATAAAGTCTTATTTCTTTTTCTTCTCTATT
GTCCAGCTTCCTACAGGAAAATAAACACTCACATCCTAAAGGTTCCAGAAACAGATGAGGTAGAGCTCACCTGCCAGGCTACAGGTTATCCTCT
GGCAGAAGTATCCTGGCCAAACGTCAGCGTTCCTGCCAACACCAGCCACTCCAGGACCCCTGAAGGCCTCTACCAGGTCACCAGTGTTCTGCGC
CTAAAGCCACCCCCTGGCAGAAACTTCAGCTGTGTGTTCTGGAATACTCACGTGAGGGAACTTACTTTGGCCAGCATTGACCTTCAAAGTAAGA
GCTGCCCCCACTTCCTAGGTCTATCAGTTAGGGTTCAGACAAGAAACAGATGGCATACTCGAGTGATTTGAGGAGAGTGTAATAAAGGGACTGT
TTACAAAGGTGTGATCACCATTTGGAGAAACTACAAAGGATAGTGCAGAACACTGGGGCTTCAATGTTGGGAGGGCAATTACCACTGTTGGAGA
AGTTACTGGAATCAGAAGGGAGCTGTAGGGAAAGCCCCACTTCCCAGGAGCTGTAGCCACAGAATAGGGAAGCTGCCACATGCAGCGACTCCAA
AGGGTGGAAACTGGATGAATGAATACCCCAACTCATTCTCCTCCCACCCTCCAATCTCCTGCTAGCACCTCCCATTGGCTGAACCCAGCTAGAA
GTCAGAGAATACAAGGGTCCACTGTTGTATTCCATAAAAGTCAACTTCTCAGGGCTCAGAGCAATATTGACATGTACAGAATAGATCTGGAGAG
GAAACAGAAAATATCTAGTACAATAGCTAATCACTGTGATTCATGCACAGTGTCATGAGCCAGCAGGATGAATATTCCTTTGCTGTACTTGCTG
CCAGTCAGCTGGTTATGGGTTTTTCCAAGAAATTTGGTCTCTAACAAAATTCTTCAGAGCCTTTACTGACTATGCTGGATATTTTTGGAAGGGA
TCCCATACTTTTGAACTTCATACAGCAGAATTTCAAACAATCTTGGGAAAATAACAACTTTTATCTGCCCAGTAAGGACAACTAACACCTAGTA
TCATAATCATTTCGTAAGAGACAGGTAATTTCATCACCGAGTGCATATGTTTTCTAATGCTCTTATATCAAATTACCACAAGCTTCACAGTTTA
AGACAACACAGATTTATTATCTGATACTTATGGAGGTCAGAAAGCCAGACTTGAGTTTCACTGGGCTCAAATCAAGGTGTCAGCAGGATTGCAG
AGGCCCTAAGGGAAGATTCCACTTTCCTGTCTTTTCCAGCTGCTAGAGGCCACCTGCATTAATTGCCTCATGGCCCCTCCCTCTATCTTCAGAG
GCAGCAACCCTATCATTCAGCCTTCTGCTTCTGTCACCACATCTCTCTCTCTCTCTTTTTTTTTTTTGAGATGAAGTCTTATTCTGCCCCCCAG
GCTGGAATGCAGTGGCACGATCCCGGCTCACTGCAACCCCTGCCTCCCGGGTTCAAGCTATTCTCCTGTCTCAGCCTCCTGAGTAGCTGGGATT
ACAGGCACATGCCAACATGCCTGGCTAATTTTTGTATTTTTAGTAGAGATGGGTTTCACCATGTTGGCCAGGCTGGTCTCAAACTCCTGACCTC
AGGTGATCCGCCCGCCTCAGCCTCCCAAATTGCTAGGATTACAGGCGTGAGCCATCATGATCAGCCACATCTCTTTCCCTAACTAGCTTGCTTC
TCTTTGCCACCTCTTAAGGACCCTTGTGATTGCATTAGGTACTCCATCCCCCTGGTTATTTGGGGTGATCTTCCCATCTCAAGGTCCATCTTCA
AAATTCCTTTTGCCATGTAAGGTGACATATTCACATGTTCCAGGGATTAGAATATGGACATCTTTGGGGAGAGCCATAATTTTATTTATCCTAC
TGAGAAGGGATATACTCTCAGACTAAAGGACAGTCCCTAGTACTGATTCAATCTGGCTTTATAGAAAATTCACTATATTGTCATTGTATTTCAC
AGTTTGCCCTTTGTCTTAGCTGGTAAGACAGAGCCTATGATAAGGACTTGTGTGGCATGCAGGTATTTAATTGGCAACCCCAGAGGGCAGAAGC
AAGAGATTTAGGAGTTTAAGAGAGGGTAATATAAGAGTATATTATCAAAGTTGTAGTGTGGACAACAGAAACTCAAATATTCAAGGACCAGCAT
GTAGACAGCCTCCTAAGATGTCTACTCAGACAAAGAATTTCAGGTGGAAGGACTTGTTCATCTGCTTCACGCCCATTGGTTGACAGGAATATGA
ACTCCATTCTGCTGCTGGGCTAGACATGCATGTGGGCTGAGTGAGCTTTCCCCAGTATCCGTAGCATCAGAAAAGTCGCAGGGCAGAAAGAAAA
GTATCCAATTTGAGGTGAATTACTGACCTTGAAGTGAGTGTAAGCCTAACTAGAATTCTACCCCAGCTGGCTGAAGTGAAAGGTGAGGCTGAGA
GGAAATAAGGCAGGACTGCACAGTCCCCAATTGTACTGTTCAAATCCACTCATGCCCTTCATTAAGTCAGCTCTGCCACTGAGCCTTCCAGCTG
GGAGGCAGCCACAATCTCTGCAGAAGATTTAATATACACCAGTTTGTGGAACAAGCTGTAGTTCCTGCTGCTGCTGTGGATCCCAAGCCACAGT
TGATATTTGTTGTCTCACTCATCCACCATACATTCCAGATTTCCCTCACCTAACACCCCAGATGGAATGGTTTCTTTGCCTGATAGGGTGACCC
AGACTTAACGACCCTAAGAGATCTGAGCTTTTGATCAGCATGCCCTTAACAGACTGGGGTTGTTGCAAATATCTTTTCATTATCACTGGATATG
GAAGTAAAAAGAACCAGTGAATCAGCGGACCCCTGAGTTCCAGACATACTCTTCCTTGTCTCCATTATGTAGCAGTGTTCAAGATTTTCATGAT
AATCAAGATTGATTTCTCCACCAGTATGGTAATTTTTTTCTTTGCTCACTGGTCTGCCAATATTAAGAGCTCAAGGTGGCCAGGCCAGTAGCAG
AGCTTTAGATGCAGTAGAACAAGCAGAGTATCCCCTGCTAGAATTCCCCGTAGGAAGCCAGACCTTTGATTCAGCAGAGCCTAAATGTGTGGGG
TTGAGTGAACTTCTATACGTGAGTCATTAGAAGTAATGGTGAGAGGGGCCAACCTGCAGCACTGGGATAGCCCTGGTACAGACGTAGAACATGC
TTGAGGAGGTTGTCAGGAGGAAATGAGTTAGACCTTGCACAGAACTACCACCATCAAGTACAGTTGGGGTGAGGCAGACATGGTATGAGTTGAG
GCATCAGAGATATCTGATGCTTTATGCCAAATTAAAATTAATTTTTTCATGGAGTGACACTGATCCACAGACCAGACTCCAAGAACTTTGCAGT
GACTAAATACCCATCTCATCATAACTTTCCTGGTATTTTCTTCTGGAAAAAATTCTTCCCTGATACAGTTTTCAGAGGCAGCTAGATGCACTGT
CATCTCTCCCCTTTTCCCACTTCCCTACCTATCCACAATTTACTACCCAATGCCAACACTAAAGTTAGCCCAACTTCCTTCTAACTAAATTATT
AGTTTAGAAGGAAAGAGAGGAGTCATGCTAAGGATCTTAACTGAAATCAAAACATTAGAGGCTGGGCATGGTGGTTCTTGTCTGTAATCCCAGC
ACTTTGGGAGGCCAAGGCAGGAGGATCACTTGAGCCCAGTAGATTGAGGTTACAGTGAGCTACAATTGTGCCATTGTACTCCACTCCAGCCTGG
GCAACCGAGTGAGACTCTGTCTCCAAAAAAGAAAAGGAAAGGGGAGAGGAGGGGAGAGGAGGTAGGGAGGGGAAAGAAAGAAACTAGAAATCCA
TCAATTTTAGGACCAACTTCAGGTAAAAAAATGAATTAGGCAAGTTGGTCTTTCAACATTCTCTACCTCTCTTTATATCATGGTTGAGACCACA
GACTTCTCACCTCATGAAAGATGAACTCTAACTAATTCATACTAAAGCTAAAGCCTCTAAAGAGGATTAAATATGAGCAATCCCACGAGAACTT
TTTTCCCCTGGAATTGTTTATTCAACTGTCGTTCGTTATATGGAATTTCCTGCCTGGTTAAGTGTAGGCCAGTACTTTGGATGAATTGTAGTTT
TCTAGAAAGACGCTTCTTATATAAGAACCTCTCCAGGGAAACAGGGGCCTGTATGAGATGAATTGAGAAATAACTTTACACCACTGATTATGTC
AGTGTTCTATTCTGCATGGTAGAGATGTGAAAGGGCAGACTGACCATTGCTCTGGAAGCCTTTACGCTGTGAGAAGTTAACAGTGGAGTAAAAT
GGCCACTCCACTCTCTTCATGGAAGCCAACATGGCTTACTAAATAGTCAACAACCATGGGAGAGACCTGTGGGGTCTTCATCAGAGCTCAGGAT
CTCCTAGGGTATCACTCATAAATACAGCCATCAGGGAGATGGAGAAATCTTTGTGCAGCCAGAAATTCTCAACCTGGTTTTACCCATCCTTCCC
AACTTTGTATTCGTCCTACTGTTTACTGACATGGATCCTCTGCTTCATTAACCATCCCTTCCTCACCACATGCTCTCTGAACTTGGCTGCACCT
TTTCTACCTCCATGCCTTCTTTGCTCAGGTTTTTCCACATAAATATCATTATTTCCCTCTCTACTAGCTCCAAGCCCACCCTCTCTCTGGGGCA
GCTCAGTCACTCCAGGGCACAAGGGGGTCTTTCCCTCATCCCACATTTTGAGACCTACTACCTGGACCATTTGTTTGCCTTGTAACTATGCTTG
CCTTTTTAATTGCTATTTTATTTTCCATGTATTTTCATTGTTCACACAAGTCTTCTTTATTCCACACTAAGGCAAAAGCAGAGTCCTGTGTTCA
TAATAAGTGCTCAACAAATGTTGGGTTGATTGGGTTGGAGATTCCATCTTAGATAATCGCAGTCCCATCATGCCAGCTACCAGACTGTGTGGAC
AGCCAGGTCAGAGCAGCCAAATGATATTCTAGCTTGTGGCACAAATACCAGCAACAAAATAACCAAAGTCACACATCTGCCTCTGAGTTCCTGG
CTTCTATTTCTCAAGGGCATTTTTAAGTTGTCTTATGACTGTTCCCTTTCTACTCATTCTCATAAATTGAGCTGTGGACTGCTGTGACCCACAA
GCTTCTCCGGAAGTCAATGTATAAAACAAACACGGAAACGAAGAGTATGGTGGGTGGAGGGTACTCCACTGACTCTAGAATGGATGACTGAACA
TTCCAAATTTCAAGCACAAGTTAGGGAGCAACAGATCATTTTCCTTTTGAAATAGGGTTTCTTCTGCTCAGCCAGTTGTTGTATTTTCATTAGG
AAATGGAATGGGACTACAGCACAAAAAATAAATATAAAAGGACCCTTGTAGGGCTGGCAGAAAAGAGAATCCTTCCTAGGAGACCTGGAGGTGA
TTCCAGGCAGTGGTTGAGAGCATGGGCTCTGATGTCAGACAGGCTTAAATTTCAAACTCTCTCTCTATTAGCTTTGAGAACCTAAATATCCTAC
TTAGCCACTCTAGCACTCAGTCTCTCATGTGTAGCATGAGGGTGAGTAGTGGTAGACAGTTTATAGGGTTATAGTGAGGATTAAATGAAATGTG
CTTATAAAGTGCTTAGTACTCAGAAAGTGTGCAAACAGTAAAAAAAAATGGTATATCTAGCAAGTTGCATGCCTTACTTGTGAGTTCATGAAGT
TGTGGCAAGGATAAGACAAATATTTTTTGCCATTGCATCATTATATCATTGCTAAGAGTATGCCATTATTGGCCAGGTGCGGTGGCTCATGCCT
GTAATCCTAGCACTTTGGGAGGCTGAGATGGGTGGATTGCTTGAGGCCAGGAGTTCAAAAATCAGCCTGGCTAACATGGTGAAACCCCGTCTCT
ACTAAAAATACAAAAAATTAGCCAGGCATGGTGGCAGGTGCCTGTAATCCCAGCTACTCAAGAGGCCGAGGCAGGAGAATCACTTGAACCCAGG
AGGCGGAGGTTGCAGTGAGCCAAGATCATGCCACTGCACTCCAGCCTGGGCAACAAGAGGGAAACTCCTTCTCAAATAAATAAATTAATTAATT
AAATTAAAGAAACGACAAAAGAGTATGCAAGAATTTTAAAACAACTTAGAGGAATATGTATGAGGATACAGGCTAAGCTACCATAATGAAGAGA
CCTCGAAATACAGTGAGAAGCGAGACAGAAGTATCTTTCGTTCCATGTAACACTCAGGTGGTTCAGAGCAGCTAAGCAGCTATGTTCCATAGAG
TCATTCAGTGATCCAGATTATTTTCATCTGTTGCTCTGCCATTCTCCAGGATGTTGTCCCTATAAAATTGTCAAAGCTCAGTCAGTGCCAAACC
CATGTTTCAACCTTCAGAAAGTAAACGAGTGGTGGAAAACACATTCAATGTTTTAAGGCCAAGACCTTGAAAACTCACTCTCTTAGCCTGAACT
TAGATTACATGGCTGGGCCCACTTAACTATAGGGGAGGCTTGGAAACATAGTCTCTGAGAAGCCATGTGTCCAGCTAATTCCCTAATACTAAAG
TTGAAAGAAAGAATGGATTAACCAGCAGTATACCACAAGGTAACAAATGACTAGGAGGATCAGGCTAGGTGGACTAGAAAAGAGACAGTCAATT
CAGTGCAACAATTCCATATTGACACTTTTCATGTAGCTGTTGCTTGGCTCTATCTAGAGAGGACTCAGAGGTAGTTTAGATAAGGCCTTTGCCC
TCCAAATACAGTCTAAGCAGACTGATTTCCTACTGGATGTTCAACTTTGGAGTCTTCAGGGATGAGTAGGGCTTCTGTACGTGGAAGAGACTAT
GAGGGAACCTGCACAGGACAAGGGTTTGCATAAAGACACTGAGGTAGGGACCTCTCCTGTTGTGGGGACAGTGAGAGGCCCAGGTCTCCTTGAC
TCACAAAGTGCTTACTAAGCACTTACTAGAAATTAAGAAGCAGATTATAATCAATATGGGTTATCCAATGTTTGGATGAGCAAGGCTCCTTATC
TTTTCTTCGTTAATGTTAATCACACTCTTTTGGATGGAGACAAATATCTGTGGGGGCTGAGCTTTGGGTCAGATAGATCTGGGTTTGCAATCCT
GGCATTGACTTTTACTTAGCAGTGATGACCTCACATAGATTATTTAGCAACTCTGAGACTTACTTCCTCAACTCTAAAATGAGGGGCTCAGACT
AAATAATGTCTAATCTCTTCTCCAGTAAAACAATCCGTGGTTCTCAGATAGCACTGTGCTGGAGGTAGTGGGGTTTGAGGGCTGGGAAGTTGGG
AGGACTGAGCCCTTCCCGCTGAGCAGTTTCGTCCAGTTTTTCCTGTACCAGCCTGTCATGTTTATTCCATGTGAATGACTCCAGAGGCAAAATT
CAAGCTTTTGAATAGGGCACAAATTAACTTGAGTACCCTTTCATTTCCCTGTAGGTGAACACTCCTCTAGCCCTGCCTTTTGTCAGTCTGGAGC
CCTTGTTCTAATCTGTACACACCAGAGGACTTTACAAGGCTTTCCCCAGCCTCCAGAATTATTCTTCTGATCCACCCTCTACTAAACTCACCCT
TTCCTCAGTGCTAGGACGTTGAAAAACCGAAACAAGGCAAAGGGCCAATTGTAATAATTCACACTAAGGCATGAGTGACTAGGTTTAGTATATT
AACACTACCTAGGATATTCTATTTCTTCCAAAAGGATCCTGTTAATCCTTGAAATTTAACAACTAATGGTATAGATTCTAAGCACTGTGAGTAC
TTGTCAGTGGGGGAAAGACATTTTTGGGCTGAGAGACTTTGCCACTGGGAAGAGAGAGAGGATGAGGAGGAGAAGGGGGATAGGGAGGAAAAAG
AGAAAAAAGAAAGAAAGATGATTAGATGATAGATGGATGGATTGATGGATGGATGGAAAAAAATAACATGAGAGAGTGGTAAAAAGCACAGAAT
CTGGAACCAGACTGTATCTTAGCTGGGTCACTTACTATGTAACTTAGGGAAAGCTTCCTAGTCTCTCTGTGCCTCCCTTTCTTCACCAGTGAAA
TGGGGATCATAGCAGAGCCAACTTCACAGAGTGGTTGCGAGGAGTGGATGAGCTGATAGGTGCAGAGCACTTAGAATAGGGCCTGGTGTGGAGT
AAATGCTCCACAGAGCTAGCCGTGTTGGCTGTCACCCACTCATGTGGCCAGCCTGTTGGTCTACCTCTTAGTTGCCATGTAACAGGATTCTGGT
GCTTTTCCTTTGCCCAGGTCAGATGGAACCCAGGACCCATCCAACTTGGCTGCTTCACATTTTCATCCCCTTCTGCATCATTGCTTTCATTTTC
ATAGCCACAGTGATAGCCCTAAGAAAACAACTCTGTCAAAAGCTGTATTCTTCAAAAGGTAAGTGAGTTTTATTCATGGTAACCCAATGCACTG
GGTGTCTGCAGCATGAGCCACTGCTTTGCACTGCAGGCCTATGGCTTGCTGCTTTCATGCTAAACCCACTCAGAGCTTATGAACCACTTTGAGC
TTGTCTTGATGATTATTTTTCCCCAGAAGAAAATGGCTCTCATCGTCAGTGAGCTGAACTTCTTACACTGAGTTTTTTAAAGGGAATGTTTTGT
TCTTATGTCTGAAAGAGTTTGTCTTATTCTTTGAGCCAAGAGCTTTCATCAGCCTCATGAGAGTGATGTTATTTTGGCAATGCAGAGAGCTACG
TGCTCCGATTTTGCTGGTGGGAGGTTGCCAGGATCCTTTCTGAGGATTCCTTCCATTTTCACCCCTCTTTTCCCCAGTCTGGATATGACCTGGG
TTAAACCCACCCCCTCTCCCAGGAATCTCAACCTCACGGTTGGGTAAGGAAAGGAGAAAGGTTTGTGAGGCCATTTGGGGATAAGGAAACAGCT
GGTTGGTGGTGCATTAACGTCTTTCAGCAGCTCCCTTCGAGTTTCTCCTTAGCCTGTTGTATTCTTACCAACACACTCCTGTTCTGTTGTACCA
GCTGGGACAGAGCATGCTGAAGCCTTTCAGCCCTGATTTCATTGCTTCATTGTTCATGTGTCTGTCTTTGGTTTCCTGGGTGGAGCCTGCCCAC
AAAACCCCCAGAATGTATGCAGGCCTAGCTGGTGCTTTCCTAAACGGCTCCCTTGTCTGCACTCAATGAACTTCTCCAAAGATCTATACATGGC
CTCATCTATAGAAAGAGAAATGACATGTGGAAATAATTCAGTAGGAGTTTGCAGCAGCACTATCTGAGGACTAGGGGAATTTTAAGTGGTTGTT
ATCTTACATTTATACTCATAACTTCTATATTTTCATCTGCCATAAAATATTGTCATGTTCTATTTGTCCATTGCCCTATGTGTGTATGTATTCA
CTTGGGTGCTGACCACAATATTTCTAACTGTAGAATGCAAGGAATTGTTGCCAAAAGAAAAAGACAGAGGGAGGGAAGAAGATGGCCAGGGGAG
TGAGGGAGAAAGAGAAAACAGGAGATGGAGAAGAAGAAAGATAGAAAGAAAAATAAATGTGGGAGGGAAGGGAGCTTCATGGTAAAGGAGGAGG
TATGTCCAACAGAACTTCGACTTTTAAATAGAACCACTTCAGAGAGTTGTGTCAGGTGCACCTCAGTTGTCTTATCTTCTGCCATTCTTCTTTT
ACCTCTCACACCCATACCTCAGGGTTCAAGGCCTGGGGCCTGAGGACTCCTTAATAACTTCAGAAATGAGCAGCTGAGTGTTCCGTTCCAGCTG
TCTTTGGGAGAATGGAATGGAGTCACACTCAAAGATAGAGTGGAAATAAATCCTCTCCTCATCCTTCACCCCAATCTTAAGAGTGAGTGAGGAT
ATCAGTAGCTCCGAGCTGGGAGGTAAAGCTCAAGTTCTAACTGTGATTAGGAGACCTTTCTTACAAATAAGAATTAAGTGAATAAATGTGCAAA
CAATTTCTTTTATATTTTTAATGAACCAGAGAGAAATCATGGTTGCCTATATAACCCTTGTCTCCAACTCACTTGCATTCAGATCTGCTTTCTT
ACATGTGTCTGCCATGCACACAAACTTGTGTGCCATGGAAAAGGGTTGAGAACTGCTGGTGATGCAGACAGAGCTTTAAACCAGGGTCAGAGGG
CTAGGCTGGGACCTTGTGCTGCCATTTGTTGGCTTTCTGACCTTGTACAAATTGCCTATCTTCTTTGAGATGGTCTTTCTATCTGTAAAAGGGG
AAGCAGCAAGAGAAGGAACATTTTACAGCTTATTGGCCGAACTTCACTGCCGCTAGTGTGGTTCAACTTGGACTACAGAGAAATCTTCCTAACT
GGTTTCCCTGTATTCACTCCTGCTACCTCCAACTTGGTCTGTTCTCACTTTTTGCTATAATAGGCTTTTAAAAATCATAAATCTACCATGTGTC
CTCTGTCCAGACCTTCTCCATGGCTTCTTATTGCTCACTGGATGAAGTTCCAACGAGCCCAGGATGGTTTGACTCATGTCTCCAGCTTTAACTG
CATCACCATCACCTTCATTGTCTAAAGCTCTAACCACACAGGATTTTCTAGTCCTCAGAGGCATGGCAGTCTTTCAATTCCGAGTTTTCTCATA
CAATATTGTCTCTTCTTAAAATATTTTTTCTTGTTGTCCACCTGAGTTGGAGTCATCTTTTAAATCTCAGCTAAGCTTATACTTCATCAAGTCT
TTCCTAATTCTACCTCCACGCACCACACCCATTACATTAAATCCCCTTATTATATGTTTCCATAGCACCTACTTCTTCTTTCAGTATACTCAGC
ACACAATCACATGTCTAGGATCTGTTTTAATAGCTTGGACTACCAATTAAATTGCATCCCTTTTAATTTTCCAATTGATTCCTCAAGTACCCAC
ATGCCCATCTTAGCAAGAAGTTCAGTGTCTCCCTCTTATAGCATGTACTTCTCCACCTCCCACAAACTGCCAGAAAGCTTACTTAGCCCACAGG
GCCAGTGCTAGGCAGCTAGGTTAGTCCTCCAGAGGGCCCTGGTTTTGAGCAGTTGCTGTCTACTCCGGCCATGCAGAATCTCTGGTCCTTCCAG
ATGTCTCCATCCACTGTGCAAAGGTAACCTTGCTGGTTCCGATCCCCACACAGACCACAGTGCTACAAGATTACAGTTCTTATGGTTCCCCAAC
ACATGCTCTGTCATTGGTCCCAAAGCAGGACCCCTATGGGTTGATGAGGTAGGAGGAGGTCCCTGCCTTAGCCACAGCTGCACACAGCCAGCCT
CTTCCCTTCTAGGCCCTCATGTTGAGCCTGGGACGCCAGTCCTAACTTCCTTCTCTTCAGTTCCTCTTAGGGCCATTGGTATCCTGAATTTCTT
AGTCCATTGCAAAGTTAAGTAAAGAAGCAGCAGGCTTGGTCCCTTTCCTTCCAGATGGCTTCTTAGCTCCTGAACAGATTTACCCACCTATACC
TCAGTGACTAGCTCTGTGTACTAAAGTGTATTGGGAGGGCAGCCATTATTGGTCCATAAAAGGTCCTGCTTACCATTTTCCCCTAAGAGGAACC
ATTCAACAGTTTGGGGCTCGAGGGTGACCTGCTGGGCTCTAGAGAAGAAGCTGGCAACTTCTGTTGCAAAATAATGTTAAATTCTGCTTCATCT
GCTTGTCTTTACGTTTCAAGAATACAGTAACTGTGTTTCTCTGATTCACCAATATATGCCCAGTGTCTAAAAAATTACCTGGCATATAATAAAT
GTTCAATGAAATTTTAAACATTGAACCTACTATATGCAGGTGAGTATGCTAGATCTGCTCTGTCCAATATAATAGCCATCTGTGGCTCTTTTTA
AGTTAAATAAAAATTCATCAGTCACATCTTAAGTATTCACACACTGTAAGTGCTCAATAGCCATGTACAGATAATTGCTCCTGTATTGGACAGT
ACAGATATACAACATTTCCATCATTGCAGAAAGTTCTACTGAAAACCACTGTGCTAGACCCTTTACAAACTTCTCATTTAATTTCACAAAAATT
GCTTGTGGCAAGGAGAAGTGTCTTTATGCTGGAGTTAAGAAAGTGAGGCTCAGAAAAGTTATATGACTTCCCAAAGTCATACAGCTGGGATTTG
AATACAGGTCTGTTTGACTCCAAAACTTGTGGCCTATTTGTTGCAAAAGTGCTTAATACAAATTGGTTCAGTCAATATTATTATCTTTGAACAA
TGGAAGGAGAAAGTAAGTTTCAATCCAAAATAATTGAGTGACTTATACATTGACTTGCTGAGCCAATGGCAAAGTCAAGTTAGAATCCAGCAGA
AGTCACCAGCTACAGAATCTAGATCTTTAGAACATGTCTTCAGATCTTCAGAACAGTGTTTCTTAAACTCTCTTGTGAAGGAACAGTTATCATC
ATAGGCTGGTAACAGTTCACCTACCAGCACCAGCCCATGAACCAGACTCTAAGTGGCACAGCCCTAGAAGATTGAGCCAGAATTTTACAGAGGT
TTAAAGACCAAATATGCTGGTTTATGGTTACCTGTGGCCCACAGAGAATGGCAGCACTAACCTCAGGCATAAATGAGGTACCCACTGAAGCCAA
CATTCAAGAGCAATTCCTATGGGTTAACCATTGGGCTCCTTTCAAATGCAAACCCTCATGAAAGAGACTACAGTGCTGAATAGAGACCTCCAAA
TTCCAGGCCAAGCTCAGGATAGTCATGAGGGAATTACTAAAAACCTGGTATATAGGGCAAAAGCAGAATTAGGAATGGACTGATTTCAGGAACC
CAGGCAATGGCAGGAGTTGGGCATTAAATCCTAAAAGAGAATCAGAGTGGGAGGGAATATGTGAAATCAGAGGTTAAGAAAAAAGTGAAAACCT
ATTCATGATTGCAAAGATATGAAACCAACCTAAGATCCCATTGACGAATAAGTGGATAAAGAAAATGTGGTATATATACACCATGGAATACTAC
TCAGCCATGAAAAAGAATGAGATAATGTGTTTTGCAACAACTTGGATGGAACTGGAGGTCATTATTCCAAGTGAAGTAACTCAGGAATGGAAAA
CCAAATACCATATATTCTCACTTGTAAGTGGGAGCTAAGCTATGGATATGCAAAGGCATACAGAGTGGTATAATGGGCATGGGAGACTCAGAAG
GAGAGAGTAGGGAGTGGCAGTGAGGAATAAAAAAGCTGCATATTGAGTTCAATGTACACTATTTGGGTGACGGGTGCACTAAAATTCAGACTTC
ACCAGTATACAATTCATCCATGTAACCAAAAACCACCTGTACCCCTAAACCTATTGATATAAAAAAAAATTAAAAGAAAGATGTGAAATCAAGG
AAACTTACTGGTGAGCAGCATCCCATTATGTGAACTTGTGCTTCTGAACCAGTAACTTGAGTTACTTTGAGCCAGTATCAGTCACTTATACCTT
AGTGCAAAATTAATTGATCAGACATTCTGACCTGGACCAGGGAAGGCAGGCAGAAGTAGCAGTCAAGACTAAAGCAGAAAAGGGAGAGCTAATT
CTGCAGCCAGACATTTCCTGGATTGAATACCCAAATTAGTCCCTCAGCCTTTAAGTGCCTGAGGGCCAGGAGTAGACAGAGGAATGGAAAGTGT
GAGACTTCTTTGTTCACACTCTTTGCCTAGGGGCCAGATTTTGCTTTATGCATTACCATCCGAAGTCCCAGGCCACAGTGAACATTTGGGCTTC
GCTATGTGGATTTATTTAGATTTACTTTTTGTCCTGCCATATTTTAATCTATAAGCCAAACAGTTTTCTCATTAATCTTATTCCATTTCTGGAA
TTTTTCCTTTTCAGACACAACAAAAAGACCTGTCACCACAACAAAGAGGGAAGTGAACAGTGCTGTGAGTAAGCATGATTTTTACTTTTCTTTC
TTACTTTCTTTTCTCTCTCAGCTTGAATTTTAAAGTAACCACTGTTCTATTAATTCATGGAAGGCAACTGAATAGTTCCAGCTTATAGAATCTT
CCTGTTTGGTAGCATTTCAGCGAAGCCTCGTTCTTAGCCCCAGAACAATCATGCCATCTTTTGCTCGGTCTATATTCCTAAGCACTCCTAGATG
ATACTGCACTGGACCTCTGGTCTCACATAGTTAGAAACAGAGTTAAAATCGAACAGCAAAGAGAAGATATTCAACTGCGATGCAATTGACAATG
GATGTTTTTGCAACAAACAATGATTAAGAAGTACATTGTTGTGGGCTCTGAGTCAAGAGTAATATGGGAAAAACACAAGTCTCTTCATGAGGTT
GACAGGTTTGGAGCTGGAATCTGTGGAGGAGGAAGGATATGATCTAGGGGTCAGAAGAAGTGGGTTACTAAAATCATTAAGCCTGGTTGGATGA
AAAGCTTAGACTCAGGGGAAGCAGCACATGATTGTGGGGGCTGGCAAGTTTGAAATGTGTAGGGCAGGCCAGCAGTCTGGAAACCCAGGCAGGA
TTTCTACCTTACAGTCTTGAGGCAGAATTCCATCTTTTCTGGGAAACCTCAGTTTTTTGCTCTTAAGGCCCTCAACTGATTGAATGAAGCCAAC
CTACATCATGGGAGATAATCAGCTTTACTTCAAGTTAACTGATAGCAAATGATAATTACATCTACAAAATACTTTCACAGCAACATATAGACTA
GCATTTGACTAAGCAACTGAGCACCATAGCCAAGATGACACATAAAATTAATCATCACAGGCACCAAGAGATGAGGGGGGCAGTCTTGGCCATA
TATTTGGCTGAAGTAAGTCAATTTGTCATTCCTGCATGAGCCTTTATAAACAGAAGTAAGTAACCAACTACTATTTGGTCATTGGAGTTGTCCA
AGAGGCCAGGGTTCTGTCTAATACCTGTTCATGCATGAACATGCCAACCTAGATTGCATGCAGACTACCAGTTTTGGGTTTTTGTTTAGTTCAG
CAGGATTTTTCTCAGCTCACTGCCTCTCAAACTTTCAGCAACAAAAGGACATCTGTGATATCAGAATCTACCACTCTAAGTATTTGGATGCAAT
AGCAATGAATATCTGAGTAAATCTAGGTGGGGAGTGGGGGCACCCTGTAGCCAAAATGATTTAACAAAATCAAACCAAAATTTTGGAAATGATG
CCTTGGTACAATGAAGAGACTACTTGAGGTAGGTTTGACTTATCTAATATCTTATTTTCTTTACCAATACCTAATGAGGAATTTAAATATTTCT
AGATAGCTTTGGAAAGGTCCCTTAAAGAGGCACCAGCATACCACTGCCAGATCTAATCCCCCCAAACACTGTTTTCATCATCATCATGTCATCT
CTTGTCTCTATAGATCATATCAAATCCTTCCCAGAGTTTTTCAGGCCTTTTGACAACTAGCCACATTTCACTAAGCCAACTCATCTACCACTCT
TCAACAAAACTTTTCCTCAAGTTGAGCTGCTCCACCAACACCACTGCCATGAGCTCATTCCCACTTCTGTGGCTTTGCTCATGTTGGTTATTTT
TTTGGAGTGTCCTCCCTATTCCTTCTTACTTGTCCCAATCCCAACTTTTGGCATGGTCTACTTTAAGATACAGTAATGAGTAACTTTATTATTA
TAACACCAGGCTTCTGTTCTAAGACATTAGAAGTGTGTTTAGACTAGCTCATTTAATCCTCACAGTAGCCCTCTGAAGTACTTACTCCCTGCTT
CCCATTTTATAGGTGAGGAAGAAAAACATGAAGAGGTTAACTAACTTGCCCAAGGTATATAGCTAATAATATAAGGGCCAATAAATTGATTCAG
CAATCCAGGTGTCCAAGTCCAGAATCCACACCCATACACTACACTCTGCTTTTTTAAAATTTAATTCAATTTTTTTTTAGAGACAGAGTCTTGC
TCTATCACCCAGGTTGGAGTGCAGTGGTATGACCGTGGCTCACTGCAGCCTCAACTTTTGGACTCAAGCTATCTTCCCACCTCAGCCTCCTGAG
TAGCTGGGACTACAGGTGCATGCCACCGTACCTGGATTTTTTATTTTTATTTTTTGTAGAACTGGGGTCTCACTATGTTGCCCAGGATGGTCTC
AAACTCCTCGGCTCAAGCAATCCTCCTGCCTTGGCCTCCCAAAGTGCTGGGATTCCAGGCGTAAGCCACTGTGCCCAGCCTACATACACAACAC
TCTCTTGCTTAATCTGTAAGACTCTCTCCCCCACTCATACCTTTTTATTTTTCCTCTGCATTGTACACACAATCTATACCACTCTTAAGCACAT
GATTACAGCGTTATTTTCTGGCTGCTTCTATGTGTCTATATTTTAGGTCCACCTGGTCAATATAATAAAGTGGGATATTAGTGTTAATGCAACT
ATATGGTATTTGATATTTGTCTTTCTGTCCGTTTATCAATGTTTCTTATAGTTTATTATATAATAAATTTAAAAGGTGTCTGATTTTGACCAAA
TTTGACTAAATACTAAAAAACAATATTAAAATAAATTTTGTTTAATTTTAGTTGAGTCTATCAACTCAAAAAAGAATAACCTACAACAATAACA
AGTTTCAGAACATTTTTTAAATTACTGATTTTATGAGTATAAAAATAAAAAATAAAAAAGTATAAAGAAAACAAAAATAATTTAGAATCCATTG
CCTAGAAATTGCCATGGTTAAGATTTTAATATTGCTCAGGCCCAGACAGCTCAGGGCTTTGACATTCCCACACCCATTCTCTGCCATCCCAGTT
CTATCTCATCCCAAAACCATCCATTATGAGGAGAGTGTACAGCTCTAGGCTGCCCGGGAGCCATCCCGCACTCTCATTTTGTGACTCGGCATCT
TGGGAGATGGAGTCTTGGGACTTAGCCTGGACATGTCCCTTGCATGTACTTCTTACAAGACTTTTATTCAGATGAATATTTTCCCTTCCAACTT
AAGAAGCACAGGGCTTGCTGGTTTTGCTTCACTAACCAGCAACTGAAGCAAGACCTGACTTGTGAAAATGCCTAATAGAGTTCAGTATTAGCGC
TGTTTCACCATTTCCTGGATCTCTTGCCTTTGTGCACATGATAGAATTGCACTTCTCTGTGATTAATTTGTAGTTAAGTGTGGTCATGTAACTC
GCTTTGGTCAATTAAATGTAAGCATAAGTGATGCGTGTTATTTCTGGGTAGAAGATGTAAGAGTTGGCATATGCTTTGCCATATTTTCTTTATC
CATCTGGCATGGTAACCAGTAACATTCTAGGTAGTAATTGCTCCATCAGTCTCAGTCTCTGAGTGACTAAAATTGACAGAGTCCCCTGCTGACC
CTCAATGTACATGGAACATGAACAAGAATAAGCTTTTGTTTTTTATATTGAGATTTTGGAGTTGTTTGTTCCTACAGCATTACCTAGTTTACTC
TAATACAACATGGAAAAAACTGGAACCTATAATAAATAGACCCTACGTTGCCATTTAAACTTCTAGTTCTGAGGAATAATAATGTGGGGAAATA
CTTTCTATATAATAAAAAAATAGAAAATTGCAAAATAAAAATATACTTATGTATCATTCATGTCCTATTAAAAATGTTATTTATAGACTCACCA
TATTCCCTTCCTCCAGAAAAATAGAAGTAAAAATATGAAAATGCCTGTAATCATGTTTTTGGATTATGGAATCAAGTATTGCTTTTTACTTTTA
TGTTTTCTGAATTTTTGTTGTACTTCACTACATTTTTGAGTGCCCTGATGTATTACTTTCAAAAAGAAGAAGAATACTTTCTGAAGCCATTTCA
ACCATCCCCACTCACCTCTCTAGATCCCAGTAACCAAATACATTATATAGGACTCTTCATCAGTCCTTATCAAGTTTAGGAAGGGCGATGCTAT
ACCTTCTTTAAAGGACACCTACCAATGTCTTAGTTGCCTTTCAAAGACTCCTAGCACAGCTAAATGTGATGGATATGCTCTAAGGATATAAGAG
CTGAAGTGACTTGCATAAGGTCATATCATAACTTACTGTTAGAAATGGAGCTAGAACTCAGACCCACTGAGTCCTTGTCTGTGACACACTGCCC
TTTCCATTTGTGGAAGTTGTTCTTGTATCTAACTTTATCTGTGCTACTATTTGGGCCTAGCCATTCTCCCTCTTATGCAGACAAGCAGATAAAC
AGTAAAACTTTAGGAGTGGATTATGATACCATAGATATATATCATCTATCCTTTACAAAATAGTTATTACAGTCATCAAGCCTTGGTTAGAGTT
TACAGACCATGTATCCTAGCTACCTCATTTCACAGATTATGACATTGGGGGCTAAGAGATATTAAGTGACTTCCTTAAGTGAAACAGTAGCAGA
CCAAAAGAAGTCATGATTCCCAGCATAGTGCTACTCACTCATTTATTCATTCATTCACCCACTCACAAACCTGTATTAAGTTTCTGTTATTTGC
AAGACCCTGTCAGTTAGGCCATGTGGGAACTAGAAGGATGAATTTATCAGTCATCCAAGATTCTTACAATTAAGTATTACCGATAAGGTACTCA
AGAAACAGTTCTCATTCACATAATTTGGGTTAAAACAAAAAGAAGCCAGCTTTCTATATACTTTTGGTCCAGTCTTTACGTTTTTTGTTTTGTT
TTGTTTGTTTTCATGAGTATCCCGACTTCCTTCTAAGAACTTCCACCTGAGAACTGACCACAGCGTCAGCATTCCACATGGGTGTGTTTCCTTT
CCCCTTTCCCATTTCAGTGGTTTCCAATTTCTTTTTCTTTTGGCACTATAAACCTTTCGCAAAGGAAATATTAGACAGAACTCCTACATGTCAA
GCAAATTAAAATAGTGGTGAAATTAGAGTGGAGGACATAATCACCCTATCATATAGGCTATTTGTCCATATCATATTTGTCCCTACAAAGGCCT
CTAAGGCAGGGGTCCCCAACCTCTGGGCCGCAGACCGGTACCAGTGGCCTGTTAGGAACTGAGCCACACAGCAGGAGGTGAGCGGGAGACAAGC
GGGCACTACTACCTGAGCTCTACCTCCTGTGAGATCAGCAGCAGCATTAGATTCTCATAGAAGCGTGAATCCTATTGTGAACTGCACATATGAG
GGATCTAGGTTGTGTGCTCCTTATGAAAATCTAATGTCTGACGATCTGAGGTGGAACAGGTTCATCCTGAAACTATAGCCTACCCCCACTCCCA
CACCCAATCCATGGAAAAATTGTCTTCCCTGAAACCAGTCCCTGGTGCCAAAAATGTTGGGGACCACTGCTCTAAGATACCTTGTGCTTCTTGG
AACATATTTGGAAAATCATGTAGCTCTCAAATTATCCCTATGTCCTGAAGCCCACCTTACCATCAATCCTCAGAAATACCCAACCCATGTCAGG
CAACTTCACACTTTCTTTCTTCAGGCAGCACAGTTGTCTCAGGGAGGGAGGAGAGTGCTATTAGCAAGAGGAGTCACTAACAGCTTCACTCACC
TGTGCCCATGAATTTTTAATGGTGTGAAAAGTCTGTGTATTTTTTATGGTTCTCTATGGCTCATATAAGAGGGACAAACAATACATGAAAGTTC
AGAGATGGGGACAAATATCACTTTAAGCTGGGGATAATCAGGGAAGAAGAAAGTTTTCATGGAGAAGGTGACTTTTGAATCAGGAAGAAATGGA
AACAGCAAACTCTTCTAGATAGAGGAGACACTAACAGGAAAGGCAGAGAGGCAGGAAGGTGTGGGAAAGTGCACGTGACCCCGTTCAGAGAGAA
AGCCAGGTGTGAGATAAGGGGGAAAGACTGTTAGGGCATGTATTGTAAACCACTAATTCCAGGCAAAAGTTAGATTTTACTTACTAAGCAAGAG
TGCTTCAGTTAGATCCTAGCAGGAAATGGAGGGTATGCTTAGAAGAGGTAACTGAGGCAAGTTTAATTTATAAAGGTGTGTGCAGCATTAAGGG
AAACCAGCAAGGGATACTGAGCATGCCAGGATGCAAGAGCAGGCAGGGAAGGTGACTATTCCTAGGTCTGAAGGAGAAAGGGGAGGGAGCAGTT
CCCAGAACCCTAGTAAAAATGGCAATGAGAAAGGTCCATCTGGCAGGACCTATGGTCTTTAACAGAGGGACAAAGTCAACCCACAACTTGTCTG
GGAGGTTGCTGAGGAATAGATACCCCAACCTCTCTCTCAACCCACTGCAACACTCTTTTTCCCCTAGACTGAGCCCAGTCAAAGACAGAGGGAG
GAGCCCAGTGATGCAGTCTGCAATGTCATCATCCTGGAGCATGAATAGAGTGCAGCAGGGTGAATAATGAGTCTGCAGGAATTAATAGAAATAT
CTGACACAATAGGGAACTATAAGAGGTTTTGAATAGGAGAGGCCCCTGAAATGTGCTCCAATATTACTGAACTATGTGTGGCCCAAAGAATGGA
AGAGGAACAGCTCTTGCAATAGGTCTGAGGAGAGAAGCTGAAGACTTGGACTAGGGCAATGGTAAAAACTGTGGAAAGAAGTTTTAAATGAAAA
GTTTTAAACCATGCGGCTTCCAGCTAGATGAACTTTTTTAAAAAAATTAGTTCCTCACTCAAATTTTGGGGAGGTTATATATTTTCTAATCATA
AAAAATGATTTTTCTTATTTGTGGGCTTTTCTCCCCAGATCTGAACCTGTGGTCTTGGGAGCCAGGGTGACCTGATATGACATCTAAAGAAGCT
TCTGGACTCTGAACAAGAATTCGGTGGCCTGCAGAGCTTGCCATTTGCACTTTTCAAATGCCTTTGGATGACCCAGCACTTTAATCTGAAACCT
GCAACAAGACTAGCCAACACCTGGCCATGAAACTTGCCCCTTCACTGATCTGGACTCACCTCTGGAGCCTATGGCTTTAAGCAAGCACTACTGC
ACTTTACAGAATTACCCCACTGGATCCTGGACCCACAGAATTCCTTCAGGATCCTTCTTGCTGCCAGACTGAAAGCAAAAGGAATTATTTCCCC
TCAAGTTTTCTAAGTGATTTCCAAAAGCAGAGGTGTGTGGAAATTTCCAGTAACAGAAACAGATGGGTTGCCAATAGAGTTATTTTTTATCTAT
AGCTTCCTCTGGGTACTAGAAGAGGCTATTGAGACTATGAGCTCACAGACAGGGCTTCGCACAAACTCAAATCATAATTGACATGTTTTATGGA
TTACTGGAATCTTGATAGCATAATGAAGTTGTTCTAATTAACAGAGAGCATTTAAATATACACTAAGTGCACAAATTGTGGAGTAAAGTCATCA
AGCTCTGTTTTTGAGGTCTAAGTCACAAAGCATTTGTTTTAACCTGTAATGGCACCATGTTTAATGGTGGTTTTTTTTTTGAACTACATCTTTC
CTTTAAAAATTATTGGTTTCTTTTTATTTGTTTTTACCTTAGAAATCAATTATATACAGTCAAAAATATTTGATATGCTCATACGTTGTATCTG
CAGCAATTTCAGATAAGTAGCTAAAATGGCCAAAGCCCCAAACTAAGCCTCCTTTTCTGGCCCTCAATATGACTTTAAATTTGACTTTTCAGTG
CCTCAGTTTGCACATCTGTAATACAGCAATGCTAAGTAGTCAAGGCCTTTGATAATTGGCACTATGGAAATCCTGCAAGATCCCACTACATATG
TGTGGAGCAGAAGGGTAACTCGGCTACAGTAACAGCTTAATTTTGTTAAATTTGTTCTTTATACTGGAGCCATGAAGCTCAGAGCATTAGCTGA
CCCTTGAACTATTCAAATGGGCACATTAGCTAGTATAACAGACTTACATAGGTGGGCCTAAAGCAAGCTCCTTAACTGAGCAAAATTTGGGGCT
TATGAGAATGAAAGGGTGTGAAATTGACTAACAGACAAATCATACATCTCAGTTTCTCAATTCTCATGTAAATCAGAGAATGCCTTTAAAGAAT
AAAACTCAATTGTTATTCTTCAACGTTCTTTATATATTCTACTTTTGGGTAAGAGGTTTGCTCAGGGTCTTGCAAGTCAGTAAGTTTAATATCC
TATTACGCCATTTGGCTCCCAAAGATTTGAAGGCCTTTGAGGCAAACTTGGATGTACTTTTCAGCAGGGTTGGGTCCTAGGGATGCTTGATCTG
GGGCTCCTGCTGGAGGAGGAGCACTTCCTAACACCACAGAGAGCAGGGCAGGAGACAAGGCCTTCTTCCCTGGAAGAAGCCCACCTATGCTGCC
CCTACCCCCACCCATGCCCCAGATATGTGATTGGCATGTTGAATGCTTGGGGAGTTTTGTTTAACTCTGTTAGGAACCCAGTGGCATAATGGCT
TCTGTCAAAATATTCCCATCTTTGTTCCTAGATTGGGGCTATCTTTTAGATTACCAAATAGGGAGAGAGCAATCTAGCTGTAGTATCCTGAAAT
CTGGGTTCTGTCAGGGGAGAAAATCATAAGGCAATGTGTCTTAGCTTCAGAAATTTTCTATACTACAGCATGCTATCTTAACACCTAACATTTA
TCAAGTACTTGCTATTGCCCAATATTGTTAAGCATTTTACACATACTATTGGAACAAATCATCCCCAAACTCTCTGAAAAAGACACTATTAATT
ACTCCATTTTACAGATAAGGAAAGGCACAGAGAGGTTAAGTAATTTGGTTAAGGCTGCTTGTTCAGTTTGAAAGGGATGGAATTAGAGCAACAA
ACCTATATTTTTTGTTACACACCATAATGTGCCCTGATTTCCTACCACATGGGGTTTAAAGTGGTATTTTTATACTGTGTGGTCTCTTTGCTGG
TCAATAATTAATTTCAGAAACAACAGATGCTGGCGAGGGTGCAGAGAAATAGGAACACTTTTACACTGTTGGTGGGAGTGTAAATTAGTTCAAC
CCTTGTGGAAGACAGTGTGGTGATTTCTCAAGGGTCTAGAACCAGAAATACCATTTGACCCAGCAATCACATTACTGAGTATATACCCAAAGGA
TTATAAATCATTCTACTATAAAGACACATGCACACGTATGTTTATTGCAGCACTATTCACAATAGCAAAAACTTGGAACCAACCCAAATGCCCA
TCAATGATAGACTGGATAAAGAAAAGGTGGCACATATACAGCATGGAATACTATGCAGCCATAAAAAAAGAATGAGTTCATGTCCTTTTCAGGG
ACATGGATGAAGCTGGAAACCATAATCCTCAGCAAACTAACACAGGAACAGAAAACCAAACACCACATGTTCTCACTCATATGTAGGAGTTGAA
CAATGAGAACACATGGACACAAGGAGGGGAACATCACCCACCAGGGCCTGTTGAGGGGGTTGGGGGAAAGCAGAGAAGAGCATTAGGACAAATA
CCTAGATGACATGTTGATAGATTCAGCAAACCACCATGGCACATGTATACCTATGTAACAAACCTGCAGGTTCAGCACATGTATCCCAGAATTT
AATAATAATAATTAATTTCACTCTTTTCCAGGGAAGTGCTATAGGAGTGTAGGTGGCGTGTGGGTTCTGGCATCAGAGATTCAAACCCCAGCTC
AACTAGGTATGAGTTATACAACTCTGAATATATTGGTTGACCTCTACGCATCAGTTTACTTCAATATAGTGAGACCTGGCCAGGCGTGGTGGCT
CATGCCTGTAATCCTAGAACTTTGGGAGGTCAAGGTGGGTGAATCACTTAAAGCCAGAAGTTCGAGACCACCCTGGCCAACATGGTGAAATCCC
ATCTTTACTAAAAATACAAAAATTAGCTGGGGATGGTGGCACGCACCTGTAATCACAGCTACTCAGGAGACTGAGGCAGGAGAATCGCTTGAAT
CTAGGAGGTGGTGGTTGCAGTGAGCCGAGATCACACCACTGCACTCCAGCCTGGGCGACACAGCCAGACTTTGTCTCAAAAAAAAAACAAAAAC
AAAAACAACAACAAAAAATGCATATATATATATATATATATATAAATAGATATAGTGATATAGTGATACCCCATCTCTGCAAAAAATTTAAAAA
ATAAAAATAAAAAAAATTTAAAGGGGCCGGGTGCAGTGGCTCACACCTGTAATCCTAACACTTTGGGAGGCCAAGGCGGGTGGATCATGAGGTC
AGGAGTTCAAGACCAGTCTGGCGAAGATGGTGAAACCCTGTCTCTACTAAAAATACAAAAATTAGCCAAGAGTGGCGGCGGGTGCCTGTAATTC
CAGCTACTCAGGAGGCTGAGGCAGGAGAATCATGTGGACCCGGGAGGCAGAGGTTGCAGTTGCAGTGAGCTGAGGTCATGCTACTGCACTCTAG
CCTGGGCAACAGGGCAAGACTCTGTCTCAAAAAAAAAAAAAAGAAAAAAAAGAAAAGAAAAGATAAAGAAAGCAGTTATCTTTCTCAGTTTATC
ATGAGGATTTTGAGCACAGGTTTTGTCATCCCCTTAGACTCCTAGAGAGCAGGGAAGGAGTGAGATTTTTGCCATGTACCTGGTACGTGATAGG
TACCTCATGCATGGTCACCTCTGAGACTCATTATGTATTGGGAGAATGGCTACTATTAAGACTTCTACTTTTTTTTTTTTTTTTTGAGATGGAC
TTTTGCTCTTGTTGCCCAGGCTGGAGTGCAATGGCACAATCTTGGCCCACAGCAACCTCCGCCCCCCACCAGGTTCAAGTGATTCTCCTGCCTC
AGACTCCTGAGTAGCTGGGATTACAGGCATGCACCACCACGCCCAGCTAATTTTGTATTTTTAGTAGAGGTGGGGTTTCTCCATGTTGGTCAGG
GTGGTCTCAAACTCCCGACCTCAGGTGATCCGCCCGCCTCAGCCTCCCAAAGTGCTAGGATTACAGGCGTGAACCACCGCACCCAGCCAACACC
TTTACTTTTTATGGCCTGGATATAATGGGCTAAAACATATATAATGACAATGCTTAAAAGACTTAGATGAATTCTAAGTCTGGAAAAAAAAGTA
CTTTTTACAAAAGGAGCAAAAATAGCATTCAGGAGAGTTTGAGGAATTGAGAAAGACCAAACAAACAAAAATCACCTATGATCTCATCATCCAA
GCACAAACACTTTCATGTTTGCTGTTTGTTTCTGCTCTTTGTCCATTTATGTATTTAAGTTTGGCTATGGTTGCAACATGTAATTTTCTATACT
TTAAAAAATTTGGGGCTGGGCACAGTCGCTCACGCCTGTAATCCCAGCACCTTGGGAGGCCGAGGCGGGCGGATCACCTGAGGTGGGGAGTTCA
AGACCAGCTTGACCAACATGGAGAAACCCCGTCTCTACTAAAAATACAAAATTAGCTGGGCATGGTGGTGCATGCCTATAATCCCAGCTACTCA
GGAGGCTGAAGCAGGATAATGGCTTGAACCCAGGAGGCGGAGGTTGCAGTGAGCCGAGACAGCACCATTGCACTCCAGCCTGGGTGACAAGAGC
GAAACTCCATCTCAAAAAAAAAAAAAAAAATTGTCATCTACCATAAGCTCACACTTATGTTGCTGACAAGTGTTCCTAATTATCACTTTTACTG
GCTGCATAATATTCTACTGAGTGGCTGTGACAAAATTTAATTAACTGTTCCCCTATTAATGTAAGATTACCGTTGTTTCTGGTTTTTTTGCCAC
TTTAGTTACAAGCTTTTGTGCTGGATATCCTCCATTTGACCCTCCAGATCCACTCTCCAGCCTTGTCCTCCCTGCTTTGGGTCCTATGAAGCTG
ACTTGTATGGACTGTATCAATGGGCTCCATTGCTCTCTGGCTTTTGATTGGGTTCAGCCAATGGGGACCCTGGCAGGAGATCAGAGGATGGGAG
AAGAGTATTGCTGGGGTATTTATTCCCCAGGCTCTCCGTCTCCAGGGTCACTGCTGGCTGGATAATTCTCTCAACTAAAGGTCACAGCTCTTGT
TGAATGACCCTTTCCACATGGCTCCATCTGCCTGTGGGTTCCAGTAACTGCTCTCTCCAGTCCCCTTTTCATGTCTGGGTGCCTACGTGTCCCC
TCACCCCCTGCAACACACACACACATGTGCAAACACACACACACACACACACACACCTGTTCCTGCTAGCCTTTGGGTACTGCACTATTCCTTA
GAGTTCCCTACACCTGGCCCACATCTCTACAAAGAGTCTCTTTAATTAGTCCTCAAATTATACAGTTCAGTGTCCTAACTGATATATCATGCTA
TTTCAGATAATGCATGTCTTTTTACATCTAATTTCATTAGGATGGATACCTGAAAAGGGAATTATGGAGTCAAAGAATGTGAACACTTCAGGCT
CTTGAGTCATACTGTCAATTTGCTTTTGAAAAGGATCTGTTGGCATTGGTACCAGTGCATCCTTCCAGCACTGGATATTTTAAAATATAATTTT
GCTAAGTCAATAGGTTAAAAAATGGTACTTTATGGTAATTTAAGTTTTCATATCTTGGATTACCCTCAAGTTTGCTTATAAAACCTGGTTATTG
CTGTGTACGTTGTTATTTTGGAGTTTGTTTAAATGTTTCTCCCTTCCCCACTCATTCTCTAGTTGTATAATCTTTCCTTAATAATTCAATTTTT
TAAAAGATCAACTTTTTAAAGCCTCAATGTCTTCATCTATTTACAGATATCCTAACAATAACACCTCTCTCACAGAATTATTGTGAGAATTAAA
TGAAAATAAGCATGTAGCATATAAGAAACATCCAAGAAACATGAATGCTTTCCTCCTTAATAATTATGCATGGAGTTTAATTAACAAATTATGT
CTATTTTTTAAAAAAATTGTGTCAGTTTTCTTTCAAGTTTATATTGAAACTTACTTCATAAAATAATATTATAGATGAGCAGAACAATTCAGTA
TTATCCAGATTTGTTTTTATATATTATGTGACTTTAGAAAATTTGTAACCCTTTTAAGCCAAAATCTTTAAAAACTATTTATTTTATTTTATTT
TATTTTATTATTTTATTTTATTTTGAGACAGGGTCTCACTCTGTCTCCCAGGCTGGAGTGCAGTGGTACAATCATGGCTCACTGTAGCCTTGAC
CTCCCAGGCTCAAGTGATTCTCCCACCTCAGCTTCCTGAGTAGCTGGGACTATAGGCACATATCACCATGCATGTGTTACCTCAGGTCTGGTTT
CTATTATACAGATCAAGTAATGCTTTGGCAGGCTACACATCTATTATCTATTTAATTCTTTTTTTGTGTGTGTGTGTGACGGAGTTTCACTCTT
GTTGCCCAGGCTGGAGTGCAATGGCACAATCTCAGCTCACTGCAACCTCCGCCTCCCGGGTTCAAGCGATTCTCCTGCCTCAGCCTCCTGAGTA
1GCTAGGATTACAGGCATGTGCCACCATACAAAAATAATTTTGTATTTTTAGTAGAGGCGGGGTTTCTCCATGTTGGTCAGGCTGGTCTTGAACT
C
CCAACCTTGGGTGATCCACCCACCTCAGCCTCCCAAAGTGCTGGGATTTCAAGCATGAGCCACCACGCCCGGCCCACATCTATTTAATTCTTA
GAGCCAACCACCACCAAAGATTATTCCTGCTGGTAAAAAATGCTGATCGCTACTCTGGGATCTCATCATCTACATTGAGATCAGGCAGATTTTC
CCACCACATCCTTGGCCTATAGAAAGCAGCTGCCTGAACATGTTCAAGGGTGCTGGGGCTTCTCCCATTAAAGCATTTTTCAATGCTTTAGAGA
AGAAACTATTCCTTAGGCTCTTTCAGGACCAAGTGTGGTGAGGGAAGAGGCACAGTTACATATAATAAATCACTCCTTTATTTCTATCTCCTTA
GAGATTCTAATGGCTTAGGCCATTAGAATCCTTCCTATACATTATTCCAGAGAAATTCTGGCAACTCAACACCATTAAATGTAACCTACCAGTG
AGTCCAAATTTCAGCCAATCAACTAAGCAAATAGTGCCATTCACAGCTGCTCTAGCTAATACATTTGTTTCAGCTAGCTTTTGTTGTATAACAA
ACTATCCAAAACTTAGTGACTTAAAACAACTGTTTATGTTTTTATGATTTTATAGGATAACAATTTGGGCTGGGCTCAGTGGAGGCAACTCATT
TGTGCTCTACGTGGTGTCAGTGGGATCATTCATGCATTTGCAGTCAGTTGGCAGTTTGGCTGCCAGATGGCTGGTCTCAGATGGCTTCATTCAT
ATCTTCCTATTGGTGCTGGCTGTTGGTAAAACCACGTATCTTCAGTAGGTTAGTCCAGGCATCTTCAACATAGAGTCTAATCCAAAAACAGCAA
AAAAGAAAGAAAACCCCAATGTGCAAGTGCTTTCCAAGTCTCTGTTTGTATCACATTTGCTAATAATGTTCCATTATCCAAAGCAAGCACATAA
CCATGCCAAAAATCAATGTGTAAAGGAATTACATGAAGGCGTGATACCACTACTGTAATCATCTACAACATTAAGTCCAAAACTATCTGGAAAG
TGTAGTATATCAGGACTACCTTAATTTGGCCCCAGTTCAATGTTATCTTCTGTGCTCCTGGGTCTAATACCTTTAGAATTCATCCCCATACATA
CTAGCCTGGTTTCCTCTAATGTTAAGTTAACAAAATCTTATGATTCTTTTGAAGTATAAGACTTTTCCTGCTGAGCTAGACTCTGCTCTTCCCA
CCTCCGTATATTCCGGGAACTGACTCTAAATAAAGGCCATGAGAGTTGGGAGCACTGGGGCTGAGAAAGAATAGGGATCTCCCTTGCAAGGCAA
GTGCCTCATATGGGCTTATTACAGGCTTTCAAACAAGGAAAGGCTAATATCATTAGACAAGAAATGAGAGGCTTCTTCCACTATCAAGGGAAAC
TCAGGGATGCTTGGGGGTTTCAACATTCTCAGTTTCATCCAAATCCACCAAAATGTCCCCATTCCTAGTCTCAGAGTCCTACTCCTTTCAAATT
ACTGAGAAAACTTTCACATATGAGACTTAGCCAGTCTGTGAATGATTGATTTGGGTTGAATTTTTCAGTCATGTCTTTCATGTCTAGTGATTCC
TGTAATTAGAAGGAATAAGAGGTACTTTCAGGGCAGTCTTAGAAGCCCTCTGGTGCTCTGAATATGATTTGAGGTGAAAGCCCTAAACTTGTTA
GTTTCTTCCTGTAAGCACCCTGGTGCAATCAGAAGCAGCCATCCCACATTACATTCCTTATAATCATCTTTACTACCATAATGGTTAAATGCAA
CAGCTACTTGGCCTCCAGAAACTTGCCTTTCATTGGTATTCAACCCTAGCAAGAACAGATGATAATTTAATCAACCATGATGTCAATGCATGCC
ATAGATTACTAGTGGCAAAGAGTTCAGCTCTCAATTCAAACCAATCCAACACTAACAACAAATCAAATCAATCCCAGAATGTCAGAATTTGAGA
ATCTGTTTCCTATGACCACACCTAGTATCAAGTGCTAGTATCAGCCAGAGTCCAGTCATAAAAATAGAGTGTATTTAATATTAGGAACTTGTAA
TAAAGATATAAGAAGACTGAAAGAGCAAAAAAGTGGATGCCAAATATTAGAACTACAGACAGCAGCTACCTTTCTACTACCTATTGCTACAGGC
ATGACACATTCAGGAACTAGAAACAGGAAATAATTCCCCCTCCCACCTTCCAGTCCCCAAGGCCTTCCCATTTACTGAACTTAACAGGAAGCTA
GCTGGCAAGGGAGGCTGGCAAATGAAGTTTGTCAAAATTCCAGCCCTGGCACATCAGAGCAGAGGAGAGGAGGATGGACTTAGAGTTGAGAACA
GGAGGTACATAACCAACTCACTCCTCAATCAAAATCTATCTGGATGAAGTGGAGACATATCCATTAAGAGATAAAAAATAAAGAGACTATAAAT
TCCAATTAGATTGTAGCACAAGGAATTGTCTTAAACTCTGTGGCATCTGACCCTGTAACAAGGGCCCAATGTATATTTGTTGATTGGTTTTATG
AGAAAAATAAAAATTGGGCATCTGCTTTGACCATGAGCATAGGACAAAATAAACTTATTTATTTATTTATTTATTTATTTATTTATTTATTTAT
TTATTTATTTTTGAGACGGAGTCTCACTCTGTCGCCCAGGCTGGAGTGCAGTGGCAAGATCTCGGCTCACTACTAGCTCCGCCTCCCAGGTTCA
TGCCATTCTCCCGCCTCAGCCTCCCAAGTAGCTAGGACTACAGGTGTGCGCCGCCATGCCCGGCTAATTTTTTGTATTTTTAGTAGAGACGGGG
TTTCACCGTGTTAGCCAGGATGGTCTCAATCTCCTGACCTCGTGATCTGCCCACCTCAGCCTCCCAAAGTGCTGGGATTACAGGCGTGAGCCAC
CGCACCCGGGCCTTTAAGTACATTTTGTAGAAGTTGTATCAAAATTCATTGGCTGCAAATGAGAGAAAACCTGATGCAAACTGGCTTAAATTTT
TAAAAGATAATGTGTTGGCTTATGTAACTGAGCAGCCAAATGTGCAACTGGGTATAGGGGGTCTAACAAATGTCTTCAGGATTCAAATTGTCTC
TATCATTTCTTCACTCTGTTTCCTTTTTGTTGATGCCATTCTCAGACAAGATATCTCCTCATAATTACAAGATGGATGCCAATGATTATTAAAC
CTTTCAGATTCAAATATTTTACAAGAAAAAAAAGCCAGAGTATTTTTCTCAGCATTCTCAGCAAAATCCTGAGATTTACAGTGGAAAACTTATG
TGCTGTGCCCTCTCCAGAACCAGTCACTGAAAGAAAGAGTATGTAAGTGATATCTTACATCTGGAGCTCCACCCAGACCACATGAACTGAAAGT
GGAGAGTGAATCAAAGTGACATCTTTTGTGCTAAGATGTTGCAATTGCCCAAATGCATCTGTGGACTTCTACTAGAAGGTATAGCAGAAGGTGT
TAGGTGTCTATTATAAGAAAAATATCACTATCATATAACACCCCTGGGGGACATATTTCTAAATTT
HUMAN SEQUENCE - mRNA
GCAAACCTTAAGCTGAATGAACAACTTTTCTTCTCTTGAATATATCTTAACGCCAAATTTTGAGTGCTTTTTTGTTACCCATCCTCATATGTCC
CAGCTGGAAAGAATCCTGGGTTGGAGCTACTGCATGTTGATTGTTTTGTTTTTCCTTTTGGCTGTTCATTTTGGTGGCTACTATAAGGAAATCT
AACACAAACAGCAACTGTTTTTTGTTGTTTACTTTTGCATCTTTACTTGTGGAGCTGTGGCAAGTCCTCATATCAAATACAGAACATGATCTTC
CTCCTGCTAATGTTGAGCCTGGAATTGCAGCTTCACCAGATAGCAGCTTTATTCACAGTGACAGTCCCTAAGGAACTGTACATAATAGAGCATG
GCAGCAATGTGACCCTGGAATGCAACTTTGACACTGGAAGTCATGTGAACCTTGGAGCAATAACAGCCAGTTTGCAAAAGGTGGAAAATGATAC
ATCCCCACACCGTGAAAGAGCCACTTTGCTGGAGGAGCAGCTGCCCCTAGGGAAGGCCTCGTTCCACATACCTCAAGTCCAAGTGAGGGACGAA
GGACAGTACCAATGCATAATCATCTATGGGGTCGCCTGGGACTACAAGTACCTGACTCTGAAAGTCAAAGCTTCCTACAGGAAAATAAACACTC
ACATCCTAAAGGTTCCAGAAACAGATGAGGTAGAGCTCACCTGCCAGGCTACAGGTTATCCTCTGGCAGAAGTATCCTGGCCAAACGTCAGCGT
TCCTGCCAACACCAGCCACTCCAGGACCCCTGAAGGCCTCTACCAGGTCACCAGTGTTCTGCGCCTAAAGCCACCCCCTGGCAGAAACTTCAGC
TGTGTGTTCTGGAATACTCACGTGAGGGAACTTACTTTGGCCAGCATTGACCTTCAAAGTCAGATGGAACCCAGGACCCATCCAACTTGGCTGC
TTCACATTTTCATCCCCTCCTGCATCATTGCTTTCATTTTCATAGCCACAGTGATAGCCCTAAGAAAACAACTCTGTCAAAAGCTGTATTCTTC
AAAAGACACAACAAAAAGACCTGTCACCACAACAAAGAGGGAAGTGAACAGTGCTATCTGAACCTGTGGTCTTGGGAGCCAGGGTGACCTGATA
TGACATCTAAAGAAGCTTCTGGACTCTGAACAAGAATTCGGTGGCCTGCAGAGCTTGCCATTTGCACTTTTCAAATGCCTTTGGATGACCCAGC
A
HUMAN SEQUENCE - CODING
ATGATCTTCCTCCTGCTAATGTTGAGCCTGGAATTGCAGCTTCACCAGATAGCAGCTTTATTCACAGTGACAGTCCCTAAGGAACTGTACATAA
TAGAGCATGGCAGCAATGTGACCCTGGAATGCAACTTTGACACTGGAAGTCATGTGAACCTTGGAGCAATAACAGCCAGTTTGCAAAAGGTGGA
AAATGATACATCCCCACACCGTGAAAGAGCCACTTTGCTGGAGGAGCAGCTGCCCCTAGGGAAGGCCTCGTTCCACATACCTCAAGTCCAAGTG
AGGGACGAAGGACAGTACCAATGCATAATCATCTATGGGGTCGCCTGGGACTACAAGTACCTGACTCTGAAAGTCAAAGCTTCCTACAGGAAAA
TAAACACTCACATCCTAAAGGTTCCAGAAACAGATGAGGTAGAGCTCACCTGCCAGGCTACAGGTTATCCTCTGGCAGAAGTATCCTGGCCAAA
CGTCAGCGTTCCTGCCAACACCAGCCACTCCAGGACCCCTGAAGGCCTCTACCAGGTCACCAGTGTTCTGCGCCTAAAGCCACCCCCTGGCAGA
AACTTCAGCTGTGTGTTCTGGAATACTCACGTGAGGGAACTTACTTTGGCCAGCATTGACCTTCAAAGTCAGATGGAACCCAGGACCCATCCAA
CTTGGCTGCTTCACATTTTCATCCCCTCCTGCATCATTGCTTTCATTTTCATAGCCACAGTGATAGCCCTAAGAAAACAACTCTGTCAAAAGCT
GTATTCTTCAAAAGACACAACAAAAAGACCTGTCACCACAACAAAGAGGGAAGTGAACAGTGCTATCTGA

TABLE 8

MOUSE NOMENCLATURE
ICSGNM N/A
Celera mCG2257
HUMAN NOMENCLATURE
HGNC PRDM11
Celera hCG25389
MOUSE SEQUENCE - GENOMIC
CATCTCACACAGTGACTTATAATATGTTCAGATTATTATGGGATTACTCGTACATAACCCTGCCAGAAGTTCAGATGCCCCTGCACAGGGCACT
ACACTCCAATGAAGGAATTGGGGCTCCTCAGTCTATCACTGGGGGAGGAAATATAAAAGATAAAACCAGAGGGCTTGTAGTTAAAAGAAAAATA
GAAATCTCACGAGGTGAAGAATTAGTCAAAGGAGACTAGGAACCAACTGATAGAAATATAAAAGTTAAAGCTGGAACAGTTCAGTAATAAAATC
AACAGGGCGCTATTGGATTAAAGTGAAAGCATAAAACAGAGATTCCCCAATCCACACTGATCAATTAGTGTGCACACTGATCAACTAGTTTGTG
ATGAGTTAGGAACTGGAGAGAGGGAGGGGGAGGGTCTCACACAGAACAATCCCGGCTAACTGATTAGTTGGAGGACTATGCCCCGCTTGTTGGT
GTGGAGTGTGCACAGCGTGGAGGAGGATGGTGTTACCATGAGGAACCTGGTAGATCCTTCCCATCAGGTGTCTAGGTCTGCCCTAAGCTATATT
GACAAGAGAGATTCCTGGCACAATGTGATGGAAATGGAACTTGGTCTCTGCCTCCTTCCACTCCACAGTCCATGACCTCGATCAGATCGGTGAA
TGTGGAATATTGAATTTATTGCTGTGTAGCCAAGGCTATCCTCAAATGGGTGATATTTCTGCCTAAGCCTCACAAGTGCTGGGGCTAGGAGGTA
TCTGCTACCACACCTGGAGACTTACACTATTCTCAATTTATGATAGGACAGGATTAACCAGTGGCTTCTGTTCATTGTCATACTGAATAACTCA
AAAGGGCTATCAGAAGGGCTTGTTGTTCCATGACACAGATATAAAACAGGCTAGATTGGCAGCTAGCATAGACCATATTAGCACACTAGCTAAA
GAACCTTTCAAAGGACTGGAATTCTGTGGACTATGCTCCCTCACAGCAGTGGAATGAAATGGGGAAGTTTATGGAAAAAGCACAGCAAGATCTC
TAATGCTTGCAAATTAACCAGCATATTGCTATATAGCGCATGGGTTCAAGAAGAAACCATGCCAAAGACACTGGGAAATACTTGGAATGAGAAG
ATAGGGAGAATCATGAGATGTGTATAAGGGTAAAATAAAATTGGAAAGTTGGAGTCATTAGAAGAACAAACCGTCAGCTATGAGGATTGTCTAA
TATTGTGAGACCCCTGCCACTGACATCAGCCAGTCTGCTAGGGCCTTTCTGCATGCCGTCTAGTTTAATCTGCAACAGCTTAAGTTAAATGAAC
ACATCCATTAAAAATTTAACTTGCCATCCGGGTAGCGGTGGTGCATTCCTTTAATCCCAGGACTTGGGAGGCAGAGGCAGGCAGATTTCTAAGT
TTGAGGACAGCCTGGTCTACAGAGTGAGTTCCAGGACAGCCAGGGATACACAGAGAAACCCTGTCTCAAAAAAAAGAAAGAAAGAAAGAAAGAA
AGAAAGAAAGAAAGAAAGAAGAAAAATTGACATTGCCACATGAAAGGAAGTTAAAGTGAGACAGTATGGAGCGATGGCTCAGTGGTTAAGAGCA
CTGGCTGCCCTTCCAGAGGTCCTGAGTTCTATTCCCAGCAACCACATGGTGGCTCACAACCATCTATAATAGGATCTAATGCCCTCTTCTGGCA
TGTGAATGTATATGCAAAATAAATAAATAAATAAATAAATAAATAAATAAATAAATAAATAAAGTGACAGTGTGGTAGAGGTGCAAAATGCAAT
CTGTATTTTAAATTAAAACAGTCCTGCACAGAGATGACAGGGTTAACCAGTTTTATCCAGTTGGCATACATTCTGAAGAATTACAGAAGGGCTG
TCTGCTTGTCTTTCCATAGTACATATATAAAACAGACTAGATTGTTTCAGGGTCAGATAGCTGTGCTGGGAATTTTCAGTGCTAATCTTGCATA
AATCCTTTCAGAAAATAGAGAAAGAGGAAATAACTCCAAAGTCGTTCTGCAAGGCCAGCTACTCCTTGTGACAAAATATGACATTTCCTTATAT
TTTTATAGGAAAGGAAAATTTTAGACACATCTGTCTCATAAATATAAATAGAAAAGTTGTATGTAAAATATTTACAAATCAAACTAAATAAAAG
GTTAACATGTGCAAGTGTGTGTATGTGTGTGTGTGTTTGTGTGTGTGTGTGTGTGTGTGTGTGTGAGAGAGAGAGAGAGAGAGAGAGAGAGAGA
GAGCTTATCTGCATGTCCACTATGTACTTGCAGGTGTGTCCAAGGCCACGAGAGGGCATCAGTTACAAGTGACAGAGCTGCCCCATGTGTGTGC
TGGGAACCTGACCCAGGTCCTTGGCAAGCTCCACTAACCTCTGAGCATCTGTTCAGCCCCATCAATTTTTTCTAAGGATTGTTTTAAACAATCT
TTCAATGTAACTTACCACATTAACCTATCAGGGGAGAAAAAACAAAAACAAAAAATAAAACAAAACAAAAAAACAAAATCAACTCGAGACACAG
AAACATCGTGAGACAAAAATTCAGCAGTTTTTCATAGCATACAGGAACAGAAAGGAGCGTCCCCCATCTAGTCTCAAGTATCTAGACAAACCCT
TAGAGAACTTTGTATTTTAATGTTCCAATAGCAAATGTGCTCTCCCTCCCCTGTCTCTGGAGTTAAGGAATGCAAAGCTACTGTCAGTGTTTCT
GTTGCAGCACTGTGCTGAGGACTGTAGCCAGTGCTCTAGAGAAGAAAAAGCATATGATTTGAGAGAAAAGCATAAACGCACTATCATTTCGCAC
ATGGTGAATGATGATGAATGCCGGAAATCCTAAAGGATTCAACGACAAACTGTTAGAATTTTAAAAAAGGGCAAACCTTCAGGTGTTGTTTTAA
AAAGGTGATGATGTGTTGTTTAAACATGGTGACTTCCAAATCAATTGTATTTCTATAAACAAGTAAAATAAATTGGAAGGCCTTTCATTTGCAG
TTGAACTGTTAAAATCGAATGCTGAATGCTTACGAATAAGTGGTATACACCCTGGATACTATGAGTACCAGCTGGGTAGCAGTAAAAGTAAAGA
TTACATGTGTATCAACTGGAGGACTTGATACTTTGTCACCAAACCAAAGATCTAATTCTACATGCACAGAAATTCAGAGAAGCCAGACTGCCAA
GCCTCACCAGACAGTAACCCTAAATTTAAAGCTACAGTGAGGAGGTAGCTTTCCTGCAGCTTTGGCTAGGAGAATGGGGTCTTAGTGTTACTTT
TGCTGGGATAAAACATGAGGACCAAAAGCAACTTGGGGAGGAAGGAGTTTATTTCATCTTACATGTTCCAGGTCATCAACCCAGGGAAGTCAGG
GCAGGAACCTGGAGGCAGGAACTGATACAGAGACTGTGAAGGAACCACGTGAAGGAATTTACTAACTTACTCTACTATAGGTTTGCTCCGCCCA
TTTCCTTTTTATCATTCAGAATCACCAGGTCGGGGGTAGCACCACCCACAGAGAGCTGATTCTTCTATATCAGTTATCAATCAAAAAATGTACT
ACAGGCTTGCCTACAGGCCAATCACATAGAGGCATTTCCTCAATGGAAAGTCCCTCTTCCCAAATAACTCTAGCTTGTGTCAGGTTGACATAAA
ACTAGCCAGCACAAACAGAGTAAGATAGACTCCAAATAGACTCACACATGTAGAGTTGGTTGATATGTATGTGCGGGGCACATATATGAAGTGG
GCTGATAATCTGGGAGGGGAATGGCAGGCATGTGTATGGAGGTCAGAGGACAATGTTCAGGAGTTGGTTCTCCATTCCTACCTTCTTTTAAGAT
GAGGGTATTACGGCGTGGCCCTGGCTGGGTTGGACACAGCGATCCTCTGTTTGGACTGAAGGTGTGTACTACCAACACATCTAGCTTCAGCCTT
CCTCCTGATGATGCAGGACCTCTGCTTTCTCTGTTGCTGTGTTGCTGCAGGCTGGCTGGCCTGCGAGCTTCTGGCTGACTCTCCTGCCTCTGAT
TTCCCTCTAGCCATAGGAGAGCTGGGGTTACATGGGCTCCACGAATCAATAGGCATTTTCACACGCTGAGCTGTCTCCTTGATTGATTTTTTGG
CAAAATAAAATTAGTGAGGAGAGGATAGCCTTTAAATGCATGTTCCCAAACTCATTTTTTATGAGATCCACAAATACCAAATATTCTAGAAAGT
TGTTTACAACTTTCAAAAATGAAATTAACCGTACATCCACCCATCACTGAGTAACCTGAATCCTGAGCTACAATAGGAGAAAGCTAATATACAC
ACATCCATCAAAACCCATGTGCCTAGCGCTTATGGTGTCTGTTTAAAGGAGCCAGGAGTTAGGTACAACCCAAGTGTCTACAAGCAGTAAAAGG
AATCACCATGTGTTCATACAATGGTATGTTCATACAAATATTATACAGCAACAAGAGGAACATGTCTGCTATTCTCAGTATGGTGGCTGAATCC
CACACATATACAATGAGCAGAAGAAGCAAACCTGGAAGTTTATGGACTATATGATTTCCCTATATGAAGTTCAAGATTGGCTGTAGCAAAGGCT
GCCTCTGGGGTATCAGCTGTATAAAATGCAAGTCTTGAGCCTGCAATTGCTTTGTTGCACAATTCAGTGTTGACTGCATAGTGATGTTCAGGAT
TTGAGAGTGAGACTTTACAGTTGCAGTTATTATAGCCCAAATTCCTGGTAAGCATGAACAGAAGGAGGATACCTGTTGGTTTGCAGTTTGAGGG
GATCCAGTTACTGTGGATGGGAGGGTTGGAAGCAGAGGGCTCAGGTCCTGGTTGTGGCTGCGGGAATGTGAAACCGCTCATTCACATCTCAGTA
GATCAGGAAGCGAAAGGAGCATCCTGCCTAGTTGCCACCTCCCTTGTCCTATTTTTATTCAATCCAGGATCCCACTGGGTTTGTCCCGGCCTCA
TTCATTAATCCTCTCTGGAAACCCAAAGGTGTGCTCCCTGGATGACCCTAAGTATTTCTTGACCTGATCAAGGTCACAAAGTTTACCATTCCAT
CGGTACAAATGGTGTGCTCACTAAGCAAGTTGGTAGCAGTAACAGGAGTATGTGTGTGCTCAGACTCTGCTGACAGCATTACAGATCAATGGAA
AGTTCTGGAGAGAAACTTAAGGAAAATCTTAAAAAGGTGGTATGGTTTGAATATGCTTGGCCTAGGAAGTGGCACTATTTGGAGGTATGGCCCT
GTTGGAGTAGGTGTGCCACTGTGGGCATGAGCTTTAAGACCTTCATCCTAGCTTCCTAGAAATCAGTCTTCCACTAGCATCCTTCAGATGAAGA
TGTAGAACTCTCAGTTCTGCCTGCACCATGTCTGCCTGGACACTGGCATGCTCCCACCTTGATGATAATGGACTGAACCTCTGAACCGATAAGC
CATCCCCAATTAAATGTTGTCCTTTATGAGACTTGCCTTGGTCATGGTGTCTGTTCACAGCAGTAAAACCCTAACTAAGACACAAGGCATTGAT
GATTTCCCTAGTAATTTCATTCCTAGAAAATTTTCCCTCAGAGCAAATGAGGATATTACCATGCCATTATTTATAATGGCAGAAATTGGCAACA
CCCAAACACGTCACAATAGGCTGTGCACAGTGCAGCCATTTAAAATCATCTTAGAGCTAAAAAAGTGCTTGTAAAAAAAAAAGAGTTGTAAAAA
GGGCTTGAAAAGGTGTTCATGGTGTGTTGTGAGACAAAAGGGTGCGTTGAAAATGATCCATACATACTATACTGTTTGTCTGCAACCTTTAGAA
TGTTTTTACAGCAAGTCCCAGGAGGCAGAGGGAAGTGACTCTCCAATCACGGGGTCGGAAGAGGGGTCAGATGCTGCCCAGTTATCCAACCTGC
TTCAGTAGCTTTACCGGGGGCATAGCGCAGTTCTTTTGAGCTATCACCAGACCAGGCTGAGGGGCAGAACGAGAACTGTGAGCAGAACAAATCC
TTTCTCTATTAAGCTGCTTTTGCCAGGGTAATTCAGCACAGCAACAGGAAAAGATACCAGGACAGAAATTGGTCCCGGGAACTGGGGCCGTTGC
TGGATAAGCCTGCTCGTGCGTTTCTTAGGCCACTGGAACTGTTCTGTGGCTGAAACTTACAGAAGAATCGGAACTTTGGAACCTTGGGCTAGAA
AAGCTCTTGAATACTGGACACAGAGATTACTGGGCAATTCTGTTGGGAGCTGAGAAGACAAGAAATGTGGAGTGTGGGGCCAGCTCAAGGGGTT
TCTGACAAGACCTCTGTCAGGAACCGGTAAGAGGACATTTGTGTGATGTTTTGTCCACATAGTCTGGCTTCATTCTGACCCAGCCCTCCACCTC
CTAGAACCTGAATGAAGTTGAATGTAAAGGTACCAGACTAACTTGTTTAGCAGAAGAACTTCAAGCCAGGAGAGCTTTCAGACCCGGGCTGGGG
AAGCGGCTCTAACTGTAGCAGATATTCGCACTATTGTGGAGAACCTCTTGATCCTACTGGGAGAAGGATCCTGAGGACAAAACCATCAGAAGCT
CTCTTTTGTGAAGATGCACTGACTGGATTCCCTTCTGGAAAAAATGTTTCCCTGGGATCAGCACACAGGAGTCTATAGAGCTGTTGGCACAAGG
CAGGCAGGCAGGCAGGCAGGCTTCATCTCAAGGGGGCAGCAGAACTCAGCAGCAGCATCCATATAATGCTGGCTTTGCGAGTATGGAAAAATAC
AGGGCTGAGGGAGGAGCTCATGGAATCTTGCTGAATATTTTCAGAGAGCTGCTAAGGCCAGGTAATGTGTATGGGGTTTGAGTCCCTGCAAGGA
GGCCAATGTGAAGCAGGGACCACAGAATACTGGAGATGCCAGAACCAAGGGACACACACTGTGAGAGCTGTGAACATGGACTGGAGCCAGCCCA
GGAGAAAACCTGTGTGTGTGAAGGCAGTATAGCTGAATGGCTGATGCTATCCTAGTCTTTTGGAGCCCAGCTGATTTTGTCACAAGCTCCGGAT
GCCAGACATGGAGCTCAGGATTTGATGTTTTCCCCTGTTTGATTTTGGTTTTGTTTTTGGGAGAAGAGGGTATCAGATCCTCTGGAAGTAGAAT
TATAGGTGGTTACGAGTCATTGGACAGGGTGCTGGGAACCAAGGCCAGATTCTCTACAAAAGCAGAAAGTGCTCATAACTGCTCCCACCCTCTC
ACCAGCCCATGGACTCCCAGCTTTTTTTTTTTTAAGTTCTACTACTTTAACAGTCAGCACTTTAGTGTTCACCCTTATGTACTGTCTGAATGTG
GTAACAGGCACATAAGTTACCATTGTGTAGAGTTTGTTTGGTGAGTCCTCCTCCTCGCTACATCTATTTATTTTTTTGTACAAATGTACACAGA
TAAGATAAGAAACATTCCTTATCCCCTTGGTCCAGATGTTTTTATTGAACCTCATTTCAATGTGCACATCTGGAATCCCCATCCTCATGGCAAA
TTTCTGGATTTGGATACCCAGGGAGCACACCTCTTGAAGCCCACATGGGTGCACTTGTAGATTTTGATCTTTGGGTCACTACCTCATTGATGGC
AGAATGGCTCTTGTCTTAGTCAGGGGTTCTATTCCTGCACAAAACATCATGACCAAGAAGCAAGTTGGGGAGGAAAGGGTTTATTCAGCTTATA
CTTCCACATTGCTGTTCATCACCAAAGGAAGTCAGGACTGGAACTCACACAAGGCAGGAGTTGATGCAGAGGCCATGGAGGGGTGCTGCTTACT
GGCTTGCTTCCCCTAGTTTTCTCAGCTTGCTTTCTTATAGAACCCATGACCACCAGCCCAGGGACGGCACCACCCACAATGGGCTGGGCCCTCC
TTCCTTGATCACTAATTGAGAAAATGCCTTACAGCTGGATCTCATGGAGGCACTTCCTCAAGGGAGGCTCCTTTCTCTGTGATGACTCCAGCTG
TGTCAAGTTGACACAGAACCAGCTGGTACAGCCCTTCTTTTTGCCACCATTCTTTGTGGGAGCCATCCTGCCAGGTTCAAGTTGGAAAAAAGGA
TGAGACTCTAGACTTTTAAAGTGTTCAAATTGTTAAAGACTATGGAGACTTTTAAAATTGGACTGGGGAGGGGGTGCTAGAGAGATGGCCTAGT
GCTTAAGGGCACTGATTGCTCTTCTGGAGGACCTGGGTTCAAATCCCAGCACCCACACCGCAGCTCAGAACTGTCTACAACTCCAAGATCTGAC
ACTCTCACATAGACACCCATGCAGACATAACACCAATGCCCATACAATAAAGGTCAATAGATTTTTTTAAAAGAACATTTGAAGTTGGACTGGG
TGTATTTTAAAGTATAACAATGTCATGAGACTTTGAGGAAGGGTGTAGAAGTTTATGGCGTGAATCCATGTTTGGGCATCAGGTTGACAAGAAA
AGGAACTAAATATGCCTGTGGGGTATTATCTTGCTTATATGAATTGATGTAGGAAACCCATCTTGATCACTGCTGAGGCCGTTCCTGGACTGTG
TAAGACAGGGAAAGCAGGCTGAGTACTAGCATGTATCTATCCCTCCCCCTCTCTGGATTCGGTGAGACCCAACTCCTTCAAGCTCCCTCCGTTC
TCCCTTCTAGCCACTGTGGATTGTGGCTAAAATCTCAAGCTTCAATAAGCCCTTTCTCCCTCATGTCACTTCTTTACAGACTATTTCCTCATGG
CAACAGAAAAGAAGCTATGATCCCATGGCAGTGAGGCGAGCTCTTGCCAACACAAATCAGAGCCTTTGTCTCTCAGCTCTGGGGCTCATCGGCT
CCTGCTCTTCACACAGATCTCACTGGGGAGGCTGGGCTTGGACCCCACTGCCACCTTCCTCATCTTTGCCACTTCCTCCCATACTGTGTCCTCC
TTCCTGCCACAGTGTCTTCACAGGGGCTGTGTGCCCTGCAGAGACTCTGCCGTTCTTCTCTTGGCAAGACTGATGTGTGCACCTAGCACATCCC
CCATGCCCCAAAGTGTCACCTCCTCAGAGAGCTTGTCCCCGGTCTAAAAATAGTCATCAGTGCTTGGGCCCCCCACCCCCACCTCCCACCTGAA
CCCTGTTGCCTCTCCTTGCTTGTCACACAGTAGAAATCCATTGACCATGGTTCCTCCAGAGGGAAGAGGCTGTCTTTTGCATTGCTCAATGGAA
TGGATCCTTAGGGGACACTTGGGGTGTTGAGTTGGGGATGTGGTTTGTGCTCCTGGGGACCTAGCAGGAGTCAGGCGAGTTGCCTGTCAGTAGC
TTACAGAAGAGCAAGAGGACCTGACCCTGGCCCTTTGATCTTAGCCTTTGCCGGCAGCTGTGTGAGCAATCCCTTGGTCTTAAGCCAGTCCCTC
TTTCCTTCCAAGAGAGAGATCCCTCAGCTGTTGGGCAAGTGGGCAAGAGTGGAACCATGCCTGGCCAGGAAGATGGCCCATGGGATCCGGGTCA
CTTCAGCAATTCTTCCAAAAGCAGAGTCATTGGAAAGATTTATGTCTTGCCATTAACAAAGGGAGACATTTTTTTTTTTTTTTTTTTTTTTTTT
TTAATGAAAGGCAGAGAAGCAAGGCTTGGCCAGTAGGATTAGACATGGGGAAACAGAAATGAGAAGGAAACAACTGCCAATGCTGTTGGGAATG
AATTTGGAATTTGGCTCTGAGCTTCCTGGCAGCCCAAGTAAAAAGGGAAGCCTGATCGGTTACAGAGTTCCTCTCATTAGCAAGGAAGACGCCT
GCTGGTTCCTCGGGGGAGATGTAATTTGTTCCAGTTTTAGATTTTCCAGGCAATAAATCCTGAGTCAAATTTCTCCCTTGAGGATGTGAGAGAT
ATCATCAATTGATGGGATAAATAATATGCTTGTCTTATAGTAAGTATCAAAAAGACTCTCCAGGGACCTGGGGGCACCCACCATGCAGCTCACT
GGGTGACAGTTTTGTTTGGGTATTCTTATTTTTAAATTTTACTAGTATTTATTTATTTATTTATTTATTTATTTATTTATTTATTTATTTATAA
GACAAGGTCTCTCTCTGTAGCACTGGCTTTCCTGGATGGATATCTAACTATTCATGGATCTCACATGGAGTTTTCCTCCTGAAGAGCTAAGACT
ATTCATAAGTCACATCTCGATGACTTCATGGGGACAATTTTGCAAGGAGAGGCAAGAGAGGAACCTGGCTTCCTCTCACAACACACTGGTAAAC
TCTGTGTGTGCAGTATTTGTGGAAGAAAGCAGCAGAAATCCATAGTTGCTTTGTGGGAAAGGGCAGCTAACTGGAAGTGGCAGAGAAGACATTG
TCTGATGGAGGGAGGCAGGCTGGCTTGGGTCTCCCCGTGCCCCCAACTAGCTGTGTGATTTGTGGCATGTTGAACATCTCTGAGCATGTTGCCT
CTTGGTAAAAAAGCCATACTTGCTTTGGAAGACTGGAAGGGAAACGGGTTAGTGGCAAAGCATGGGATGTGTGTGCGTGGATGCCATGCACCTA
CTGACTTTAGCTCCCATCCTGAGGTCCTCAGGGAATTCCAGGGAGCACCCTCTCACCCACATTTCTGGGATTCCGGCCCAACTTAGTGAGAGGC
AAGCTTGAGACACACACTGACCTTGCCTTGGCCACATGTCTCTAGGGGGGTCCCACGAGCCCTGCCAAATGGTTCAGACCGGAGGAAACTAGTC
TCCAAGGTCTGCTCATCCTCCGGGCCATTGGCCATACCCTTCTTTTCCTGTGCCTCTTTGTGGTCTGTCTCAGTTTCTTCGTCCCACCCACTCT
CTGCAGCAGGCACTAGTCACTGCAGGCCCTGTGCCAACCCCTGGGCACTTGGGTCTTCACTGTTTGCCTTCCTGGCATGTGGTCCTGTGGATTC
CTGGCCCACCCCTGATGTTGTTTCATGTATGGTCTCTTCCCCTCATAAGGGACAAGTGGCTAATTCCTCAGCCTCTCTTCCTGAGCCTGCCCTC
TCCCAGGTGTTAGTTAGAACTTGAGGTAGTCCTACAGACTTCTGACTGGGCTGGAGCCACAATGGGATACACAGATCCAGGTCTTCATCCATAT
CCCACACACGACCCGGTGCTTCAGAAAGCTACACTGTGGACACACAGCAAGCACCTAGCCATTTCCATAGCATTACAGCTGCTTCTGGGCTCTT
TACATACACCCTCAGGGCTCACCCTAGAGCTGGCAGCTGGACCTTTACAGCTTTGCAAGAAGAGGAAACAGGGTCATAGCTACAGACACTCAAG
GTCATACTGCAAGTGACAGACAAGGGAGCCGAAGAAGCGTTTATGTTACCAAGGTCAAGAGAAATAAGAGAAGTTGCCTCCTGTCCCAGCCCCC
ATCAGGTCTCCTAAGCAAAGCATCCTCAGGAGTACTTGACCTTGGAAAGCCCTCTGGGATCGCCCTGGTGGAAGCTCCTTTCCCTGGGTAATCG
GCTCCCCTGAGGTGACACCTTCTTGGGACTATCTGGACCTTCCTGGACTACCTGGCACTGGGCAGGCCTTCCAAGATCTCTTACTCAGGTAGAA
TCCTTTTTGGCAACTTCCAGCCTAAGGTGAGGCCCTCCTTCGGCCAGGGAACCCACCCTGCAGCAGCCTCCCCTTGGGGTCATTACAAAGAAGG
GCTTGGGGTAGGGTTGTTCTGGTTCCCTCTGACCACTCACACTTCCTTTAAACACCCCAGCCTTACTACATCCCTTCACTATCTTTCTTGCTGG
GTGCCATTTTGCAGATGGATACAATGAGGCACTAGGCAAGTTAAGTTAGGACACAGTCCCGTTCCAGCTTTGTATTCAGATTTGGTACTGTACT
TTGATCTGAACAGCAAGTGACACTCACCCTTTAGTCCCCACGTAAATAGTTAATTTTTCTCACAGGCCCCAGGGCCCTTTTCCCTTGGTGTTCT
ATGGGACGCATGTTACCCAACTTCAGTTGATACTTCCAAACGGCTGAGCAACCAAAGGGTTAAAGGGGCCCGGAAGAGGACCCTGTAAGGGTTA
AAATTATAAACGCATTTATCGCCCTCTGACTATTTATTGGGTTGTAGACCGACAACGTCGAAGCCGGGCAGAGCCAGGAGGCACCCTCGGATGG
GCTTGCGCAAACGCCACAACTTTCTAGCACGACCCCCAGAAAGTATCCCAGGCACAAAGTTGGCGCAGCCCGCTCCTCCTCCGCAGCGTCGCCC
TCCCCGTGGGGCGCGCGGCGGGGTACGCGCGAGCTCCCGCGCGGGGCCGGCTCGGGGGACGCGGGGCTCAGCGAGGCTCGGCTCCGGCCCCCTG
AATTGGGGGGTCCGCGTCTGCCCCAAACGCCGCTTTCTTCCGTTTTCCATGTCATTTCCTGTACTAATAAGATGGTGGTCAAAGCAGGGGAGGA
GAGCAGCAGCGGAGTCGCCGCCGCGCTGCCGCTGGCGCTGAAAGTGGCCACCGTGGCCATGAACGTGAACATGGCTTCGGAAGACGATGGGCGG
CTCGCCAACCGGCGCGGGGGCCGCGGCGAAGAGCGCGCGGGGACACTGGGGAGCGGGGGCGCAGGCAGGCGACGGGGCACGGACCGGAGCACCG
GAAGCGGGGCGGGTCTCAAGGTGGCCCGGGCCCGCAGGTCGTCGCCACCGCCCGACGCGGGCCTCGGGCGGCCCAGCCTCCGCCTCCTTCCTCC
GCGCACTCGCCCGCCTTGTTCCACTGTAGCCTTTTCCTCGGCTCCGCCGCGCGCTGACGGACGCGGCCGCCGGCCAATGGGAGCGGCTGTTCGG
GCCCGGCGTTCGCAAAGAGAGCGCGTAGACAGGGGGCAGGGCGGGGCGAGGGCGGGATGCAGCCGCTGGCCTAGTTGCAGGGACCCGGTCGCCG
GTGCTTCGGGGCCGGGCTGGGGGGCTTAGGGACCGCATGCCTCTCTGTGACCTGCCGCGGGCCTGGGGCGGCAGAGGGAGGGCGTGCCTTTTAT
CTATTTTAAGTCTTGGTTGCAGCACGGACTCTTCGGGAATTCGCCCCTGAGTGTCGTCTCCACCCCCCACCTCCTCATTCCCGGTCCCCAAGGT
ACTATGTGATGCAGAAACTGCAGAAACTTGGTGGCCAATGACCTTCCTCTGTCAAGGCCAGCGTGCCCTAGGCAAGTCGCTGCAGGGACTACTG
AGCAGCCTTGGGCTAATAAGGCAAAAAGTTGCTAGTGTCTTGAGCCCATCTGAGTTGGAGCTCAGACCCTTAGAATCAGTTGATGACCTAGAGA
AGGAAAGTCTGTCTTTTGTTTTTTTTGACCATGGCTTCGTGTGGAGGACAGCCGTGGTTTGTGATTAAGAGTGATCAGAAAGAAGCACGCCCAG
TCCTTTGTGCGTGGGGACTGCGAGCAGTGAAGGGAGCCTTCTTTTCATCTTGAGAACCCTATAAACTTTGGGAGTTTTGTCTATCTGAAGCAGA
ATAGTTTTCCATGAAGTGTGATTCCCGTGGCACCCCTGTCCCCCGAAGAGGAAGGCAGGGACTTTCCTAGGTCTTGGTTGATGGTGTAGGGTGT
ATGGGTCAGAAGTCCCATATCCCCCTCATCCCTGGGGAGCGGAGTATGGTCTCTGCTGGGAACCTTCTGGGCATATGGCCCAGACCCCCTGGTT
TATTTAGTTTCTTGGAAAGGACATCTTCAGGATCCTTCCCACGGGGCTTTGGATTTTGCAGGGCTGTAGTTGATCTCTTCAAGGGGATTCTCCT
TCCCTGCAGTGGGTCCAGTACAGCCTGTGTGGGAGTCAGCTGGGAGAGCATTGAGATAGGATCAATTAAGTACCTGGCCCAGGGTCAGACTCCT
GGTTTCAGAATCTAGAGCTGTGATCCTGGGCGATGCTCTGTGAACCATTTTGGGAATCACGATTGCCAGACCCCAGCCTGCTCCACCCAGCCCC
TAACTGACAGTGCCCCTAAGGAGCAGATGCTCTCAGGGTGTGGTACCTTCTGAGTATCCGTCTCAAAAATGAGGAACACTTGTCCCAGGGGCCC
TTGATCTGTCATCACGGCCATCACCCCCAGCCTGGAGCTTTCTGGTCCAAATGTTTCTTAGGTTGGTTCTCCGAGGTGTCTGATGAGAGTCCTT
CATCTGGCTTACTCTTCACTTAGATTCAATGTGGGATTATCAGAAAGCCTGAAAGAGGCAAAGACTATTGGAAGAGGAATTGGGGGGTTGGAGT
TATGCTGCCTTTGTGGTCTCTCACTTGGCTTTCCTCCATAAGCTTCCTCCAGGGTTGATCTCAGTGTGTCCCTGCCTGCCTCATCCAGTCAGTC
CACCCATCTGCAAGCCCAGGGAGCTCTCTTTAGTGAATCCAACCCATCCTGGCTTCCTAATTTCTCCCCACTAGCACACTGGGGAACAGTACTT
CCTGTTCCCAGGACAACTGCTAGGGTCTGAGATAAGGCACCTGCTCTTGCCCTGTTCCCCGTAGTACAATTGGGGTCACCTCAGTCTCCCTAGT
TCCTGCTCCCGCTCCCCCTCCAGTCTGCCTGGTGCACTTCTGATAAAGGATCAAATTGTTGCTAACTGTAAAGCCTATAACATGACTCTCCCTT
ATCTTCAGCCTCATCTCCTGCCCTTCTCACCTCTCTGTTGCCCAGCCCAGCCCAGCCTGCCCTGGCCAGCCTCTCTTGGATCCTGACCTAGGAT
TTATGAATGAATGAAAGACAGTGAGGGGCAGAGTGGTGTGGAAATGTCTGCTGGCCTTTACTTTTTGTTTTTGTTTTTAGATTTATTATGTGAG
CACACAATCACTCAACACCGGAAGAGGGCATGGGATCCCATTACAGATGGTTGTGAGCCACCATGTGGTTGCTAGAAATTGAACTCAGGACCTC
TGGAAGAGCAGTCAGTACTCTTAACCTCTGAGCCATCTCTCCAGCCCCTGTATCTGGTGGTCTTTATTGCCCTCCTAAAAATATTTCCAGCAGC
TCCAGGTATACCACAGGGTTGCCTTCAGTCCTCTTATTCTAGGTCAAGGCCATGTGACTAGCTGGTGAGTCTGGGACATGAGTTGATTGTGGCT
CAAAGCAGTTAGCTGTCAATGCAAGACTCTCCTAAGATCTCCTTCTTGGTGGTGTGGCATCTGATAGCAGAATTGGAACTGCTTCTGTGTCATT
CCATGAGCAGTGTACCTTGGTGCTCCACCATAAATGTGCAGTACAACTGAGGGATAGACCTTTGTTGTTTGAAACCACTGAGACCCAGGTTGGC
TTATTCTTACAGGATAGCTTATGTCACCTCCTCCAGGAAGCCTTTCTGGGTTAGGCTAAACTTGGGTTACAGGTGTTTCTGGTCTCATCCTCGT
CTCCTTGGATTCCTGTTGTTACTTAGTGATGATCTTTCATCGTCACCAGACCATGGGTCCTGTTTTAGGTAAGAGGCTTGTGTCTGAGAGCTGC
TGGGTGAATGCCCTCTGTGAGTACTTGGCCCTGACTGAGGACAGGCAGTGTGCTCCGAGCCTCCCTGAGCTGCAGTTCCAGCAGGAAGGTTTCA
GATGCAGCAGTAAGTCACTGGTCGAGGGCAGATGCAAAGCTCTTGGTCTTTGCAGGTGTCTGGACCCAGTGGCACTGAGCTCCAGAGAACTATG
ATAGGCAATTCCAGGAGAGATAGAGGATGAGATGGGAGGCCCCGAGATCTCTGCTGACATAAGGGGTGATTCAGTGCAAGGGTGGGAAAGGAAT
GGTGGTGATATCAGCTAGTGTCCCCGAGGGCTTATACCTGTCAGGCACTAAGCAGCTCTGTCCTGTATAAGTCAGTTTCTCAAGACTGTGTATC
ACCTGAGGCCGTTTACTGTCTGAGGCAAGGGCATTCATTTAGCTAACAGTTTTGGAATCTGAAGGTCCAAACAGCATGGCCCCAGCTCCAGGGA
GATGTCTTTGGAGTGTTTCATTGTGCTGGATAGCACAGTGGAGGGGCATGTGACAGGGGAGATCACATGGTAAGACAACAAAAGAGAGAGAGAG
AGAGAGAGAGAGAGAGAGAGAGAAAGAGAGAGAGAGAGAGAGAGAGAGAGAGAGAGAGAGAAAGAGAAACCCAGGGACCTCCTAATAGGCCCCA
TGTCTTCAAATCCTGCCTCTTTAAACATGTGAACCATTATCACTAGGGTACACTCAAGTGCTAGCCACTCCATACTACATCCCTGTTCTGGAGA
TACCCACAAGTGTTTTACAGATCGGGAAACTGAGGCTCTGAGAGGTGGGGTGAGAGGTATAGAGCTTATGGCACAGTCAGGATCAGAAAGTGGG
ATGTCACCTGAAAGCAGCCACTTGGAGCAAAGGCAGAGGACCCTGGGAGCTCCCTGGATAGGGTCCATACACACACAGACACACACACACACAG
ACACACACACACACACACACACACACACACACACACACACACACGGAGACAGGGGAGGGGGGAGGCATCAATGTGGAGAAGGGACCTGTGGCCA
GCCTTGGAGAGGCAGTGGCTGGTGAGTGTGTGTGGGTGTGAGAGGGTGGAAAGGGTATATCATAGAGCAATCTCTATGAAAGTGTCGTGTGGGG
TGAGTGACCGTTCTGGACACCCCTCTAATATAGACTTTTGAGCAGAGGAGTGCCTGGCCCAGGAGTACCTGGGAGTGGGGCTTGAGGGAATGAA
GCTGGCTGGTAATCTCCACCTCTGGCTGGAGCCTGCAAGTCCTTGGAGGCCCCAGAGCCTCATGTTCTCAGAGTGGGGCCAGGGAGGGAGGGGC
TTCTCGGCAGCTGGCCCAGGGGCCATGGCGGAAAAACAAGCCGTGCAGAAAGAAAAAAAAAGCCAGAAGAAAAAAAATGTGTGTTGTTATTTTT
AAAAAGCCTATTTCCTATTGCAGATTTGTATCTGCTGATACCAAGGGGGCTGATGTTCACACGCACAGTGTACCTACACTAGTGATATCCCTCA
GCCCAGCACCCCAAAAAAGGATTGCAGGAGGGGAAGCCTGGGCCTGGGTGTCATCCTGCCCTTTGTTCCTACCTGAAGCTTATATATTCTTGCC
GTGCTAGGCTTGGGGCCACCCAGCCCTTGTCTTACACAACTCATAGGCCCGCGCTCAGCTAGGCACCAGGAAAGGTGGTCATAGGGTTACAGTT
GATGGGGAAGGGGAGAGCTGTCAATCCCACTCAAGGAGCAGTCTCCTCAGTGGAGGACACCTTCAGGATGGGCTTTGAAGGATGGGCTGGACGC
AGAGGAAGAGAGAGAAAGAAGAGAGGCGTCTGAGTGGGTCAGGGCTGGCGGGCAGCCTTGGAAAGATGGTCTGTGGTTGGTGAGTGTTGGTGCA
GGAGGGTGGGATGGATATCAGAGCACAGTGTCCTGGTCCTGCCCTTGGGCATGGGCTCCCTAGAGTACTGCTCCCAGTTGGCTCTGGACTGGCT
CTGTCTTCAGCAGCAGGGCTGCCAAAACTACTGCCTTCTGGGACACTTTCCTGTTTAGAATTGCAGCCCGCACGATTCCCTGTCTCAGCTTCCT
GCTTTCTCTCTTTATGACAGATCTTACGCCTTCCTTACATGTTTTGTCTTTCTCTCCCTCGCTGTTCTTAGCCCTGTATCTGGCACTTGGTTTA
CCCCTGTGAACAGTGTAGGGTTTTAGTGATTGTGGCCTGGAAGCTGTGTGAAGTTGTGAACGAATGGGGAGCGTTCACCCTTCACAACCTGCGT
ATAAGGGTAAAATAAAATTGGAAAGTTGGAGTCATTAGAAGAACAAACCGTCAGCTATGCGGATTGTCTAATATTGTGAGACCCCTGCCACTGA
CATCAGCCAGTCATACTCCAGGGCTCTGCTAGGGCCTTTCTGCATGCCGTCTAGTTTAATCTGCAACAGCTTAAGTTAAATGAACACATCCATT
GAAAATTTAACTTGCCAGCCAGGCAGTGGTTGTGCATTCTTTTAATCCCAGCACTTGGGAGGCAGAGGCAAGCAGATTTCGAAGTTCAAGGCCA
GCCTGGTCTACAGAGTGAGTTCCAGGACAGCCAGGGCTACACAGAGAAACCCTGTCTCAAAAAAAAAAAAAAAAGAAAGAAAAAAAAGTGTTTC
CCCAGCCTAGGGTTTGAGACACCGAGGCTCAGCAAGGGGAAGAGACTTCACTGTTGGTAAACTGTGCAGCTGGGACTTGAAGCTGGGTCACCTG
GCTCCTGCCTTACCTAACCCCCAGTTGCCAGCTAGGCTGGAGTAGCTGTGCTGGCCAAGAAGGAGTTTGTGCACTGGTGGGCTGCAGGGAGCCC
CTGAGGGATTTTATGAGACACCGTGACCAGATCCTTGGTCAAGAGTGCACAGGCTGCATAGAAGTGGTTGCAGAAGTGATAGAGAATGAGGAGG
AAGCCAGCAGAAGAGATGATTACAGGTTGGAGTGGACAGGTAAAGGCTCAGCTCAGGTGGGCAGAGAATCCTAGTGTTCGTTGTTTGGTTCGGG
AGGACTTGGATCTGGGGCCCGGCAGGGAGTGGGAAGTTGGGAAGGGACCAGGTTGGTGGGGTTGGCATTGTGTCCTGCAGAGGACCTGTGCCAC
CTTCCTCTGTGAACCAGGGAAGCGTGTCCTGGGCATGGACACAATCTTGTATTCTTTACCAAGGACAACATACCTGAAAAGAGATGGACAGAGG
CCACACCCTCGGGCACTCATTCCACACACTGAATGCTGCTTGAGGCCTGTCTGGCTCCAGTGTCCTCTGGCCACAGAGATAAGGGTGTCAGTCC
CGCCTCATATTCTGTGGAAAAAAATCCCTTTATATTGTTGGTACCAAATTGAAGTCACTGCTCCCCAGGAGGGGTCGGCTGCTGGAAGGAACAT
GTTTTAAGTACCATGGTCCTTGACAACAGTGCTTTTCACATCCATTTATGGAACCTGGGCTCAGAGAGGTTGATTGAGCAGCTGAAGGGCACAC
AGCTTAGACTCTTTCCAGTCACCCCGTGAAAGCTACATCACAAAGGAGGGCTTTATGTCTTATCTAATAATTCCCAGCAGGCGACTTTGGCGCT
TTAAAAACGATTTGATAGGTAGGGTCTTGGCACAGATGTTGTCAGGACCTTTTTCCACCCTGGGAACCTCAGAGTCAGAACATGGAGTCACAGT
TGGCTAGACCCAGAGTCTCAGATGAAGTGATGATGGGGGAGGGGAGCAGACATGGGCAGCTGGCATTCTGCCTCCTGGCATCAGCTTCAGAGTA
GAGTAGCAGTGGAGGGCTGGGTTCTATCTTTCAATGAGACACCTGAACCTCTGGAGCTCTCTGTGGCCTGTGGAATTTTCCCTCCCCTCTGACA
CAGAAGGAAACCCAGCATTCTCTTCCAGGTTCTGCCTTTTCTCAAAACATTTATCCAAAGTCCCAAGGCAGGCTGGCTACCCCACAGGACACCC
CATTGTGCCATTGAAGAAACTAGTGTCTTAGTCAGGGGTTCTATTCCTGCACAAAACATCATGATCAAGAAGCAAGTTGGGGAGGAAAGGGTTT
ATTCAGCTGACACTTCCACATTGCTGTTCATCACCAGAGGAAGCCAGGACTGGAACTCAAGCAGGTCAGGAAGCAGGAGCTGATGCAGAGGCCG
TGGAGGGATGTTACTTACTGGCTTGCTTCCCCTGGCTTGCTCAGCTTGTTTTCTTATAGAACCCAAGACTACCAGCCCAGGAATGGCACCACCC
ACAAGGGATCCTCCCCCCCTTGATCACTAATTGAGAAAATGCCCCACAGCTGGATCTCATGGAAGCATTTCCTCAACTGAAGCTCCTTTCTCTG
TGATAACTCCAGCCTGTGTGTCAAGTTGACACACAAAACCACCCAGTACACTGAGGCTCAGAGAGCAGAACAGACCTTATAGGAATCATTTGCT
TTCGAGAGGCCAGACTCCAAATCCAGCCTTCCTCTGTGCCCAAGGTTCTCCCTCCTGGATGTATTTGTAGTAACCAGTGTCCATACTCAGGAAA
GGGTTGTTAGTGAACACAGGAGAGCAGGCTACGGGTGGTATATGGTGTGACTTAACCTTGAGCTGAGCTGGTGTGGTCATATCTGAGAAGGTAG
ATGTGGAGACACCCCCCACCCCCACCCCACATACAATCCCAGAGCTCCCAGTCTGTTATCTCCCCCTTTCTAGAACTTTCCTAGAGGGAAAGGA
TGAGGGCTAAGAAAGGGTCCCGCTGCCAGCTTCCCTCGGTGATGGAGTGGCTGGCTTGGGGTACAAAAGAGGCTGAGTAGACCCTCAGTTCCTC
AGCGAGGGGGCCTGTTATGAGAGAGCTTGTCATATGGCAGCACATGGGACCGAGCTACAGTAGACCCAGCATTGTCATTGTCTGCGATAGGGTG
TGACCTGGAGGGTGGGGAGCTATTGATTTGACCTGGCTCAGACCTCACAACCCCAGCAGAGTCAGGGTGACTGGTCTTGTATGTGTCCCTGTAT
ATCTGTGTGCCTGTAAAGCCCCTGTGCAGACTAGCAGGGTTCCAGGAAGCAAGTCAGAGAAGCCAGGGAGGTTGATGTTTGCCAAGGGTTCAGT
GTCCCAGCCAGAAGGTGTGATGAAGAGCGAGAAGCCCCTTACCTCAGGAAAGGGAAGAGACTTGTGACTTAGTGCATGAGAGAACAGGACTAAT
CCCTCCCAAGAAATACTGAGCCCCAGTACCAGCCATGCCTGCAGGAGGCACATGCGTGTGCACAGCCCTGCCAGCCTCAGCTGCCCCACTGTGC
CCATGTTATTGGATCTGGCTTGTAAAGGTGAAATGGAAGCCCAAAGAAGAAGTGACCCAGAGTCACACAGATAGGAAAGGCTGAGCCGAGATCA
CCCACTCTGGGTCACTGCTGGTGGTGGCTGGGTTGAGGATCTTTGCACGGGGTGACTGGTGGCTTTCCCGTTTCACCCCTGAGCTGCAGGGTAG
TGAAGCGAAAACATAGACCATGAAGAACCGTAGCCCAGGCCAGTCTGGGGCATGGAGGACTCTTGGCACAGTGCAGCTGCATTGTGGGGTGGTT
GGGCCTATCTACCTAACACTTTGCCATCATATAGTTGACCAGTATTCCCTTGGGGTCTGGGAATTCAAGCACACTGCTGGATGGCTCTCCTTGG
AAGGCAAGGGTGACAGCAGACTGGCCCATGCCACGCTGGGAATGCTGGGTGGACCTCTGCCCTAGGTGAGCAGCTGCGAGTTACTGTTCTGCTA
CAGATCCATCAAGGGAGTGAGTGTCACAGGACTTGGAGAACAAACTCAAGAAGCTCTTACTGCACCGTGAGGTCCCCCTGCCATTTTCACTGTA
GTTTGCAAACGCATCCAAGGGCCACTGTTTGGAAGCTGAAACAAGGTGGTTGAGGTTTGTTGTTGTTTTGGGGTTTTTTGTTTGTTGTTGTTTT
TAATCACAGGTAGTTTGAGCCATTCTGGCTCCCATCCTGACTATAAGTGAAGGCAGGGAGTGTGAGGATTGGCGTTGGCAATGGGGAAGAGAGT
GCAGGTTCACTTCCCAGAGGTAGGGGAGCAGCTGAGCACCCCTCAGGAAAAGCAGAAGTGTGTCTGGAGAGGGGAGGGAAGGGCATTTGGGCAC
TGAGAACAGGTATGTGTAGGTAAGCATTGGGAAGGCCAGAAAGCCATTGAATGACAAGAGGTCTTGGCATTTGAGAGAGGGGAGGGGATGACCC
TAGAGACTGGAAGAACTTGCGTGGAGCTACTGAGGAGGTCTTGATCATTCCTTAGTAGAGGTGAGATGCAGCGAGCTTTTATTTTAGGTTGAGA
ACTTCTGTCCCTGCAGCAGGAGGCTTGGAACGGTTAGAGGAGGGACAAGACCACCCTGGGGTGCGGTCCTTGTTTTTCTCTTGTCTGGGAGGCT
GTCCTTGCACTCAGTGGAGTGAGTTGCCAGGGAGGAGCTATCTGCCCATTCTATGGGGCAGTCCTGGGGTGGTGAGCCAGCAAGGCCCCTAGGG
TGAGGCCACAGCCCTCCTAGGCTGATCTGGTCCTGAGCTTGTGGTTGCTGGCTGTGGGTGAAGCTTCATTCCCCCACCTCTAACACTAGAGCTG
CAGGGTCTGATCTGGAGAGCAGCCAGTGGAGTGGCCAGGTTCTTTGCGGCCTGGTGGGGCTCGTGCACGGAGGAGGGAAGCTGGGGCTTGTTGT
AGAGACTGGGGTTACCCTGGTTTGTGGCCTACCATCTGGAGAGCTGGGGTCAGAAGGGACAGTGGCCTGCAGTTGTTTCCCATGTTGATTTCCT
GATGGCCAGGTCTGAGGCCAGCATTTCTGCCACACCTGGAAGCTAGGCCCAGCCTGTGCCACTTCAAGGGAGCACAGCGTACCGTGACGAGGTT
ACTCCTTCAGCTTTCTCCTAGGCTCTTTTCTGAGCTTGAGGGACCCACCTTGCCCTTGGTTTCTGGGAAGAACCCAAGGTGTGCCTGGGCCTGC
ATGTGGCCACAAGCCCAAGGAGGGGGGCTGCCCAGGAGGGAACTGTGAGAGCACCCCTCCCACTAGCACCAGCCTCTCCCTTCTCCTTGGGGCC
CCGTTAAGGGTGCCCCTGGCTCACTCTCTCTGTTAGGCTCCCAGACCAGCTGGGTCCTGCAGCAGCCGCAGGCTGGGCAGCTGCTGTTCAGGGG
AGTCTGCGCTCCTGCAGAATGGGGTGGGGCAGGGCAGGCCTTCTTCACCTGACCCAGCTTGGGCTTCAGGCCAACCAGCCTGGAGTCACTGAGG
TGCCTTGGTTTCGACGTGGCTCTGGTCACACTGGAAGAGTGGCGCTCTGTGTGTGTGTGTGAGGGGGTGAGGAGCAGATCCTAGGTCGGTGGTC
TCAGGTTTCCCCCTGCTGCCTGCCCACCCTGAAGCTGGCAGCTTCCCTGGTTAATGTCATGCAGGACTGCCTAAGTCCTGTTTCTCAAAGGACA
TCCAAAGCCTGCTCGGCAGGGTCCCCCAATGTAATCCTCTTAGGTTGCACCCAGGAGTCAGGGCTCAGTCAAGGGCTTGGTTTGCCTCAGGCAC
CATTTAGTATGCCCCAGTAGCAACTCCCCGCTCCTCTCTTGTCTTTGCTTTGATTGTTTTGACACAGGGTCCAACCATGTAGTCCTGGTTGCTC
TGAAACTCTCTAGGGTGGCCATCAGGGTGGTCTCAAGTTTACAAGGATCTGCCTACCTCTGCCTAGAAAGAAAGGCACATGCCACCACACCCAG
CAAGTCTAACTGTCTTGGAGCCCAGTCATTGCCCATTTATTTTCTTGGTGCCTTGGGTCTCCCTAGAGTGTGGCCGTGACAGCGCGTGTTACTG
GAACCACTGTTTTTCACAGAGGGGTGAGCTGATCTTAACTTATTCATAGCCAGCCTGGGAGTTGTGCCATTGACCCAGTTGGCAGATGGACTAA
CTGAGGCTCCCAAGAGTTCAGTGAAGATGGTAGGGTCTTGTGTGGGTACTTGAGGGCAGAGCTGAGGCTCAGGTCTACTCCTCCTGGCTCTTCT
GTCTACACTGGAGTTGGCTTGAAGTAGTGATGTCATCAGTATTTGACTATTACACGGTCCTGACCTGGGGTTTAGCTGGGTTCGGTCTGTTTGG
GGTTAGGATTCACTGGACAGTTAGGACCCTTGCCTGGGTACAGTGTCACCGTCAAGTACTTAGCCTATGAACACTTCAGATGTTTGTCTGCTTT
TGCCTCTCTCCTTTTGGAACTTCACCTTTGAATCATTCCTTTCAGATGCACCCTCTATCCTGGAACACCCCAGAAGCTGGGCAGGAGCTAGGCA
GCCTCTGGTAAGGAGGCTGAGGTCACTGTGGAAGCGGGGAGGGGGCCCTGGGTCCCTGCTTCCCTAACCGGCTCGGCAGGGACACCCAATCCAG
CTGTCCATGGGGCATTAGACTTTTCTCTGATGCTCCCAGCCTTGTTGTAGGTGATTGTATTGCTGTGGGGTGAGGTGAACCACCATTTCCCAAG
GCAGATCTTTGTGTCTTTTAAAATTTGTGGTGCTGAGTTTTAACCCTATAAAGGTCATACAACTATATATTTGCTGTAAAAGTGGAATGTTGAC
AAATAAGGCGTTCCTTAGCAGACGGGGCATTCCTGACTCTTCCACAAGGCTAGCATCACAGTAGCCGTGGTCCTGCACAGTTCTTTCTGGATTC
AGTCTTGTAGTGTGTAGGACACAGACAGCGAGAGTTGCCTGTAGTATTGACTATTGGGTACATTAACTTTCAACATTCTACCTAGAAAAATCTG
GCTTTTCTGTAGGGTGTGTGTGTGTGTGTGTGTGTGTGCGTGCGTGTGTGTGTGTGTGTGTGTGTGTGCTTGCACTTGTTGGGTGTGTGTATAC
ATGTGCTGGTGCATTTATAGACCAGAGGTCAGCACTGGATATTTTCCTCGGTCACTTTCCACGTTGAGATGCGTGGCTGCCAGGGTCTCTCTCT
CTCTCTCTCTCTCTCTCCATGGGGCATTATACTTTTCTCTGATCTCCCAGCCTTGTTGTAGGTGATTGTATTGCTGTGGGGTGAGGTGAACCAC
CATTTCCCAAGGCAGATCTTTGTGTCTTTTAAAATTTGTGGTGCTGAGTTTTAACCCTATAAAGGTCATACAACTATATATTTGCTGTAAAAGT
GGAATGTTGACAAATAAGGCGTTCCTTAGCAGACGGGGCATTCCTGACTCTTCCACAAGGCTAGCATCACAGTAGCCGTGGTCCTGCACAGTTC
TTTCTGGATTCAGTCTTGTAGTGTGTAGGACACAGACAGCGAGAGTTGCCTGTAGTATTGACTATTGGGTACATTAACTTTCAACATTCTACCT
AGAAAAATCTGGCTTTTCTGTAGGGTGTGTGTGTGTGTGTGTGTGTGCGCGCGCGCGCGTGCGTGCGTGCGTGCATGCGTGTGTGCGTGCGTGC
GTGCGTGTGTGTGTGTGTGTGTGTGTGTGTGTGTGTGTGTGTGTGCTTGCACTTGCTGGGTGTGTGTATACATGTGCTGGTGCATTTATAGACC
AGAGGTCAGTACTGGATATTTTCCTCGGTCACTTTCCACGTTGTGATGCGTGGCTGCCAGGGTCTCTCTCTCTCTCTCTCTCTCTCTCTCTCTC
TCTCTCTCTCTCTCTCTCTCTCTCTCTCTCTCCTAACCTGGAGCTCTCTGATTTGGCTGGGCTGACTGGCCAGCAAGCCCAGGGACACTCTCTG
CCTCCCCGGATTTCAGGAACTCACTGGTGCACCTGACTTATTTTTACATCCCTGGGGTTTGAACTCTGGTCCTTATGCACAGCTAGCACTTTCC
CAACTGGGCCGTCTTCCTGTTCTCCCCCGCCAGCCCCCCTCTCTACTATGTAGCCCAGGCTGGCTTCCAGCTCGTTGGTCTTCCTGCCTCAGTG
TTCTGAATACTGAGATTATGGGCTTGAGCCATCAGAGCTAGTCTGGCTCTGACTTCACATGGAGACCTACCATCTTTCTAAAGACACTAATGCA
TAGTGCTTGGAAGTCATTGTTATTTAGCCATTCCCCTACCGATAGACATGGAGGTTAGCTATGATTTTATTCCAGAACTTCCTTGGGCCTCTCT
GGAGTTGCTCTTGCTGGGTTATGGGAACCTGCTGTGCTTTAAAAATGTATTATCCAATATTTAAAATGTGTCCGTTAAGAGTTGGTGAAGGACC
AAACACCTCCCATGGACGCCAGTTATCCCAGGCTCACTTCTTTTGAGCTGGATGGAAAATCTGCATCCTTGACCCCAGTGTCTCCTCTCCTTCA
GTCTCCTCTGCTGTCCTTGTTCTAGTGGGAGCCGCAGGGAGGCGCGGTGGTGGTGGGTGTCCAGAAGATCAGCAGCCTCTGGAATCGGAATGCC
CAGGCTGAAATCCTGACCCAGCTCCTCCAAGAGCCACCATTTCTAGAACTGTGGCACCTCTGTGGCTTGGCTGTATCCACTGTGAACCAGAAAT
AACAGCACTCGCGTCATTGAGCAGCCTTGAGTGCAAAGGGCTGGCCAGGCAAAGGGTAGGCAGCCAGCAGTGTCCCTGTAGTGGGGTCCCGCCT
CTGCCTTCGTTCACCATCGCCTCACTGGGCGCAGAGCCCACGTCCCTGGTGAGCAGCCTGGGTTGGCTCTCGCCTTTGTGACGCCTCCATTCCC
CGGTCAGTGGGTGTCCCAGAGGACATTGCTGCCTGGGAATGCAACGGGTGATCAGATTTCTACCCTGCTTGGCCCTGAAGGTTCTGGACTGGGG
CCTGCAGAGAGGGAAAGTGGCTGTCCTGGTCATTTGCTCTGAGGAGGTGGCCAGGAGCATAGCACCTAATGTAACCCCACACCGTGGACACATT
TACTGGTCCTCAATGGGGCCGTTGTGGCTGTGGATTTGATCTCTGTTGGCCTGCATGGTGGGCAAGAGAAGAAAGCAGCGTGGGGCCAGACTTG
CCCCCTGGGAGCTGGGTGTCTTAGCACCATGTTGGCCCAGGCAAGGGTTTGCCTCAGCCTTTGGAGTACAGCATTCTCCTGGTTCCGTGTTGAG
AGAATGGCTTCTGACTCTTCCTTACAGCCCTGGCAAGCAGCAGAGTCTGCCCTGCCTGACCAGCTTGCCTTGTGAAACAGGTGCCCCGGTTGTG
GCCGTTGCCCAGGAGGAGAGAAACCAAGGTTACAGATTCCTAAATGACAGTCTCAAAGCCACCCACATGGTTGACCACTGGCAGGGCTGGGAGT
GGTAGCCAGGCCATCTGGCTGTCTATTCACTCCTTTCTTACAAGGCCACCCAATGTGATCATTTTGGCAGAGGGTGGCCAGCCGGGCGGCCTGC
TTAGCCTTGCCTCTCCGGGCAGTGGCGTGTGGTCAGCGAGTGCAGACAGCTGGGATTACATTTTAAAAGCAGAGGAGTGTGTGGCTGGCCCCAG
CCCAGCCCTCCCCAGCACCAAGCTATACACCCTCCTGGTGGTTGAAATGGCCAGCTGAAGAGTCGCTGCTCCAAGGTCCTCTGCCCTGTTAGAG
GGAGGACACTGGGGACTCCCAGGGAAGCACAGAAAAGCCATCACTGGGCCAGTTTTCAACGGGGAAAAGTAAGTCTCCGAAAGACTAGTGGGCT
AGAGGGTGCTGGTGGGTAGTAGGGCATCTGCCTGCCCCCTCTGTCCAGCTGCTGGCTCGTGCTGGTCTGCTGTCACCTCATTCTGAAGGTACTG
ACCTGGCCCTGCTCAGAGTGAAGGTTGTAAGCATAGTGTCAGAGGGATTTGTACACGGGTTTCACACATCGCTTGTGTACTGAGCGCTTACCTA
TGGCTAGATGATGTTCAGTGCCTTGTGATGAGCCAACTACTCCTCAGAATAACCCATGATGGCCGCTGTTGTTAGTTCCCATGTGTAGCTGGGA
AAGCCTCACCACGTAGCCCAGACTGGCCGCAGATTCACAGGGCTTCTCTTGCCTCCGCCTCCCAAGTCCTGGAGTTACAGGTGAACTTGGCTAG
CCCCATCCCAGTCTTCTAGCACAGGCTGTATGAGCAAGGGAGCCATCAGACTGTGGAATAAGGGCTTTTCTGGGAGGCAGTGGTGACTGGGGGG
GTCTTGCGGGAGTTACGTGGACTCTTTGCACGCCAGTTTTGATGGGAAGAGGCCCAGCAGTGCAGTTTGAAAGCTCTGTACCCAGGGCCCCGGC
CACAGCAGCTGTGCTCACTCTGGGAGACCTGCAGCAGAGCTGATTGGAGCTACATCCTAGCGGGGAGGTGCTGCTTCTCCCAGCTGGCCATACA
CACGCTCAGCTGCCAGCCCCTGGAGTGGAAGTACATTCTCTCTGCAAGGGCGGCTGTGGGTGTTACTGGTGCTCTGGGACCAGAGGGACAGAGG
GCAGATAGTGTGCCCACCTTCATACAGGACACTTGTGTTGCCATTGAACTCTTTCAGCCTACAGTGCTCGCTCCCCCCCCCACCCCCCGCAATC
CCCTCCCCGCCCCAAGCCTCCACTGAGCCTATCTCTAACACAGCTCAGGAAGGTCTGGTAACTTACCTGACTCACTCGCCTTCAGATGAACCCC
TCCTTCAGTGGCTCCTGTGTTCCTCATGGGTCCCACAAATAGAAACAGCTGCCTAGATGAGAGCTATACCCAGGGTCTCTACTGGGTCTGGTTA
GTTCTATGAGTGACACAGCCACATAGGCCCCTGTATGCAGGGAATCCTTCATTCCCTCAGCCACGTTTACTCTGCACAAATAGCCCAATAGCAG
GATCTCTACAATGGTATTCACACTTGCTCTGTGGCCTTTTGTAAGCCCCACTCCCTGGGTCAGTAAGGGATCTCCCCCCACCTTGCCAGTGGAT
AGAACATGGAGGGCTTGCTGTTGGGTCTGAGACTGGAAAACACATAGTAGGTATGAGGGCTGCCAGTGTCCCCTTGTCACACTCACCTGAGGGC
TGGAGATCAAGGGGTCTGGGTTCAGATCCTGCTTCGTCGCATCCCTGTAGTTGCCCAAGTATCCTCTCGGAGCTGACTCTCCCTCACCTGTGAA
CTCGGAGGGCAGCAGAATGGGCAGCTCACTGGAACCCAGGCATGTCTGCTTGCTGGAGGTCTGCTGCCAGGGAGGGAACAAGGAGGGACTTAGG
AAAGCTCTAGAGCCCGTGCCAACAGCCCAGGAAGCAGAGGCCAGAAATAGAAGAGCCCGGAGACAGGGGCTCCAGATGGCCAGGCTGCCCTCTG
CCAACCCCAGAGGCCAACCCAAGCCCATGTACAGTCCTCTCTCCAGGGGCTGGTCACAGCATAAATTACTCCTTGGCAGGGCAGACTAATTTTA
CCTTCGTGGTTTTCATGATTCATTTCCCATTCGGGGACCTGTGAGAGAAATGGAAAGGACTCTGCTAGTTGGGTGCTAGCATGCTGTGTCCGTG
TGGGGTTCCCCTGGGCTGTCATGATTTGGTATAAATGGTGCAGTGGATCCCAGGGCTGGAATCCATCAGGGTTTTGTTTGTTTGTTTGTTTGTT
TGTTTGTTTACCATAGCTTATGTTGGCTTCTGTGGTAGAGTAAGGAGGTTGATGGGAAGGTGTTTGTCACTTGCTGGTCCAGTGCTGTGAGTTT
TTGGATGCCGCCCCCCCCACCCCATCCCACCCCTACATTGTAGTCACAGAAAGCCAGAGCAAGCAGGTAATGAAGGAAATGAAGCCCAGTCGCC
TCATTGTTCAGACAGGGAGAGTGGGGCTCAGCGCCTAAGTGGAAGAGGTCCCAGGCTCACAGCAGCACAGCCCGACTTGTCTCATTTTATAAAG
GGACAATGGGAGGCTTCTCTAGTTCTGATCACAGCAGGGCGTGTCCCTCTGCCACTTGCCTTCCCCGATTCTACTTGAGCTGCTCCTTGCAGGG
ACCTGGCTGCCTGTGCTCTCATTTCCGGCCTCCTGTACCTCTGGGAGCACAAGATGTCATCCACTTGAGCTCCGAGTCTGCGGGGCTCAGGGTA
GGGAGCAGTCCAGAGGAAGTCGAGCTGGCAGCTGGATGCATCCCAGGTCCTCAGTCTGCCATACAGGTCACCAGCTCGGAGGCCACCACAGGAA
ACTCAGAGGCTGGCTGCCTGGCTGCCTGCCTTCAGAGCTGGGGAGGCTGGCTCATCTGAGCAGGGCTCCCACTTGCGTCCAGCACACCGCCACT
GTGTGGAGCCACCTGCGCAATGTGACCAGCCTGTCTGACTGTAGAGAGCAGACACCCCAGGCTTTCCTGAGGGTTCTTCTGTTTCTTTAAAACC
GGCAGAAAGAAAAGTTGAGATAGCAACAGCTGCCAGTGTGCCACCTACATATACTTCCTTCCTTCCTGTTGCCTCAGGGTGGAGCAGGTTCGAC
AGTGATTTTCTTGTGGGATCCTCTCAGGAAGACCTGTCCTGGACAGAATCCCTTCCTCTCTTAGTTGCAGTTCATCACCTGTCCTACAGGGGTG
CCACCTGAGAATAGTAGCAGGAGGGTAGGCTTTGTGCTGGTAAGATTGCATATGGCTGCTGTGCAGCTTTGGTCAAGCCACTTAACCTCTCTGA
ACTTCCATCTGAAAAGCGAAGGAAAACTGTTTGCAAGGTCGTGGACTTTATGGAATCACCACCTGCCAGAAACTCAGCCGTCACGCAAAAATGG
AATGCATGTGGCTGAGCTCGAGGTCCAAGAGGATCCTGAGAGCTTCAGGCAGCATTGGCTCCAGGGGCTCGTACAGTGTCTTCAGGACACCGTC
CAGCCTTCTCTGCTTTCTTCTGTACTGGCTCTCCTCACAGGCAGCCACTGGGGTGGGGTTGTGGGTGGGGGTGAGGCACAAGCCTGGCAGCAGG
CACAGCTGTTCTGCATCCTTTTCTCTTCGCAGTGGTCTCCTGGGACACTCCACCCCTTCATCCATTGGCTGGGTCTCCTACCTGTCCACCCTGA
GGCAATAGGAAGGAAGGTGCACTAATCACTGAGGACTGCTGATCCAGGCTCTGACTGGTTGGTCTTCACAATCACACACCTAAGTGTCCCCAAG
ACACCAGACAGTCGGTGCCAGAAAAAATAAGGGTCAGTAAGGCATTCCCATAGGCATAGCTGCTGACCCATGAGGACAGGAACCCTGTTCTCTG
GATTCCCAGCAGGTAGGACAGTGCTGGCATGCAGAACTGCAGGAGGACAGGCCACAAGGGAGGGAATGGGTGCCCTCCACTGGGTTTAGAAGAC
ACCCCACTCTGCCAGGGCATTGTCACATTAGAGCGTCCCCCCTCCCACCTCCTTCCTGGAGGCCCGTGAGGAAGGTCACCTTCCTGGCAGTGTC
ACTGCTCTGCAGAGCCTCTTCCTTCTGAGCTTGTCAGCTCCACCAGTGCAGTCCCACAACCGCAGTCTGTCCCACAGGTGTGACAAGCAGACAT
TACGAGTGGTAGTGCCCGTGTCACCATACATACGCAGGTTTATTAGCCGACATCATCCCCCCTTGGTCTCCTTCATCTGTAAGCTTCAGTAGCT
CCCCACGCTGCTTGTCCTTTTGAAGCAGAGCTCCTGCCCTGTCACCCATAGGAAGGGAACAGTGAGGAGTGAGATCATGTGTAGAGTAGGTGCT
TGCGAAAGGGCAGGGCCAAGCCAGGATCTCTGCCAACTCATGGATCTGTACGTGAGCCCTTCCACCTTCGTGGAAAAACTGCTTACTCTGGTGT
TTATACATGACTACCACACACACACACACACACACACACACACACACACAAGAGTCATAGAAGAATCCCAGGACCATAGTGTCTGTACTAGAGT
ACAAAGGATCAGAACAACTTTATTGCCAGCAGCTCCGCGCACAGTAGGCTCCCCCTGCATGGCAGATAACTGCATTGGCAGGACAGGTAGATCA
CCCTGCCCTCAAATCACAGGGAAGAATGTGATGAGCATACAGTGGGTCGCTGCGTAGGGGAAGAGCCCAGGGTTGAGGGCTCAGCACCTTCCAG
GGCAAACAGAGAAGCCAATGTCTGTCTTCTGGAAGCCATCAGGTACATGCTCAAAGTCCCCTTGACCTACTTTATTACTATGTGACCCGCGGCA
GAAATGGTTTGGGGGCCAGAGAACAGGAAGCCCTTACAGCCAGAGATGCTCTGTGGAGATGTTGCAAGGATGCCCAGAACTCAGGCGGGCAGCC
CTACCCCATCTTGAGCATGCACTTGGACAGTCCCCTAAACCAGGCATGTTAGACAGACTGTGGGTCAGGAAGTCGGGGGTGGAGCCTAGGGTTC
TGTTTAACCAGCGAGTTCACCTCTCACCTCACTTCACCAGCTGTCCAGAGCCGCTCGGTACATGCGAGTACATCTGAGTATCAGAGCCATTCTA
TATGTGCTGCCAATGGCATCAAGTATTTGAAAATGGATTTCTTTGTCTATGTGTGTGTACGTGTGTGTGTGTGTGTGTGTGTGGGTGCACACGT
GCCATGGTGTATGTGTGGAGGTCAGAAGATAACTTGAGTCAGTCCACTCCTTCCACTATGTTATGCCTGGGGATTGAGCTCAAGTCATCAGGCT
TGGCAAGAAGGACCTGAAGGGCTTTTAACTCATTGAGCTCTGAGTACATTTCACACAGCAACCTTACCAAGTAGATGCTGCCCATGTCCACAGG
CCCAGAGAGATTCAGGCGCCTCCCCAGTCGCACTACAATAACTTGAAGCATCATGGAATGCTCTTCTCATCAGCCATTCTGTTTCAGGTCTTGC
TGTCACCATGGAGGAGGCCGTGCATCCCAGAGCCTGTTGAGTGTCCACTTTGCACCAAGCCCTGTTCTGGCTGCAGGGGACACAGCAAGGGACA
GAATAGATAAGCATCTTCACTTCCTGGAGTCTACATTCTAGTTGGAGAGAGACCGGGCCGATCTTATAAATAAATACCATGCACATTGGTTGGA
TAACGGTGTGGTCGGCAGAAAGTACAGCAGGCAGGAGAGAAAGAGCAAGGGGTACTAGTTAATGTAGAATGCCCAGAGATTTGACATCAGTCGA
AGCCTGGAGGGAGGGGCAAGCCATGCGCATCCTCTGGAAAGAGCACTCCTTACAGAGGAAATAGCCACCACAAAGTCATGTGGGAGGGAGGGCA
GGCTCCTGGGGCTATGCTAGGGGTAGTGGGAGAAAGAACCATGAGTGACGAGGTCGCAGGAAATGGGTATGGTGTCCTGTATAATCTGCTCTGC
ATGAGATGGTCACTGTGCAGGGTTTGAAGCAAGGGGCCCCATGGTGATAGATTCGCCTTGACTGTGGGTGCCCAGTAGGGCGCCACTCTGGGGA
GGCTGCTATGTGTGAGTAGCTGAGTTCAGGAAGAGTCACTGGGAAGGAGTAGCTTCCTTCTTTAGTAAAGCTGCCTGATGTGTTGATGTGACGG
AGCTCCCAAACCCTAGGGAAGCTCGGAGAAAAGGAGAGAAATGGTGGCATGGAAGCCCACGTGTAACTTTTACCTGGTTTCGTGGTGTGAGATC
AGATCACACTATGTAGCAGGGGCTGCCCTTGAGTTCCAGATCTGGGGTGAAGGTGTGAGGTATCACATCCAGGTTCTGTGCATCCTGAAGAGGC
CCCCGTACCAGGTCCCTCTCCGTAGAGTGCCCAGTGCAAACTCCCAACCCAGAGAGCATGCATCTGAGTTGGTTTCCAAGGCCCAGACGGCGAG
CTGAAGCCACCAGGAACAAGCCTAGTCAGAAGGGGCCCCGTACCTGCAGTGGGGCAGGGCAAAGCAAGGGTGGGTGACAGCTGATCAGAGCGGT
GACCCTGTCTTCAGAGGCTTCAGAGAGAGAGAGAGAGAGAGAGAGAGAGAGAGAGAAGCTTCCAGGCAGTGAAGAGGTGGGCTGGCTTTCCCAT
CTGCTGACCGGCACTCCAGCTTTGCCCATCGAATTCCCCCCTTCCAGTGTGTCCAGTACCAAACAGTTCTCAGATCCTCCAGCCTCACCTGTCC
ACACTGGCTTTCCAGGCTCAGCACAGACTCCAGAGGACTGGGGCTCAGTCTCACGGGACGTCCACTTCAGACTATTTTTTTCAGATCCTAAGTG
TCTATGCTTCAGACCACTGTAGATCAAGGCTCCGAGCCTTCCTCAGTCTCTCTCCATCATCCCCTGGGTTGATTTACAGAATGAAGGAAATGGT
GTTTCTCACCATTCCTGGCTTATAGAAAAGGCCCAAGCCAGACACTGCCAGATAGAAGAGCTGCATAGGGAATGGTGTGTGCATGCATGTGCGT
GCGTGCGTGTGTGTGTGTGTGTCTGTGTGCGTGTGTGTGATACGTAGCACATACATCTGTGCTCTGGGATGCACATGTGAAGACTGGGGGTTGA
CGTCCACCCCCATTTCTGTCCACCTTCCTTTTGAGACAGGGTTTCTTGTTGAACCTGCAGCTCACTGTTTCAGCAGACTGGATAGCCTGCAAGT
CCCTAAGCTCTGTCTGTCCCCTCCCCCACAGGACCCCATACTCGGATTGGGTGTGGGTGCTGGGAACTGAACTCAGTCTCTGTGCTAGCCCAGC
GCTTTACTCACTGAGCACTTCCCCAAGCCCATATTTTAAAAAATTGTTTAGTTTAGTTTTCTGGCTCTGGTCATTGGATCCAGTGCCCTGTGCA
TACCAGGCAAGCACTCTGCCTCAGAATCATACCCCAGCCATGTGAAGTAGGGCTTAATCTCCAGCCCCTGTTCCCCAGTTTCAGAGCTCAGTGG
GTGAGGCTGAAAGCTCTAGCACTGGCCCCACCCTGAAGCTCTCTGGAGTCCACAGAAGTAAACTCAGGTGCTGGCCAAAGGGCTTCTGAGGAAT
AACAAGAGATATTCCCTTTAGAGAGTGTGCCAGAAACCAAGGACAAGGTCAAATATGTTTCTTATTGTACCCATGTCCTTCCGCTCTGTTGGCT
GGCTTCATGGATGTGCCCTGCAAAGCCTTCTGCAGAGCTTGGCTGCCTTGGCTGCCTTGGCTGCGGGCTTTCTCACTGAAGTCACAAAATAAAC
ACCTTTCACTCACTTTGACTACCCCAAAGTGGGTCAGCTGTCAGCTGCAGAGGAGAGAAATCGTGTGTAATGATATTCCTTAAACACCAGGTTC
TTGCTGGTAGGGATGGCGCACGCCTTAAATCCCAGCACTTTGGAAGCAGAGGCTGCCAGATCTCTGTGAGTTCGAGGCCAGCCTGGTGTACAGA
ATGAGTTTTAGGACAGCCAGGACTACACAGAAAAACTCTTTCTTGAAAACTAAAGCCAAACCAGAGAGCTGGCTGGAGAGATGGCTCAGGGATT
AAGAGCACTGGCCGCTCTTCCAGAGGACCCAGGTCCAATTCCCAGCACCCACATGACAGCTCACAACTCCAGCTCCAGGGGACCCAGTACCTTC
ACTCACGAATGCAGGCAAAACACCAATTCACATAAAATAAAAAGTAAAACCAATCATAAAAAAGAAAAGAAAAGAAAGAAAAAAAGGCCAGGCT
TCATGTCCAGTCGCCGGTAAGACCATGGAGGTGTACCATTGATTGACACGGTGGCTCCTGCTTCTGGGGCTGAGAGTGGCCCAGCTGGTGCTTG
AAGGGAGCTGTGACACCTGAGTCTGTCAAACACAGTGGGATGCAAGCCCACAAAAATAGTGCCACTCTGAAACAGCTTCTTCAGGGCTTGTTCT
GACCTCTGCCTTTCTGAGTCCTACCAACAGAAGAGAACATGTGGTACATTTCAGAAGTCCTCCCAGAGGGAGCACCTTAGGAGGAGCACAGTTC
GACAGGCCCAAACAGTTGGGTTCCACAGCAGGAAGCCCTGAGCTGACTAGAGCTCAGTTCCACAGTTGCCTATGGTGTTTCCTGCCTTTGGGTC
CCCTGCCTTAGGTCTTGCCTTTGCATGCTAGTTCATACAAGTACACACACACACACACACACACACACCTTACAGAAGCATGACAAGAGATACT
TCCACAAATATACCCATGGAACCAGCACCCAGGCTGAGAAATCAAACCTGATTTTAGCACCCTAGGACTCTTGTTTTCTACCTATTCCAGTTGG
CTCCAAGAATAACTCTGAACTTGACCCCTACTTTACTTCCTCTCGTAGATGTGACTACATACCTGATAGAAACAATTTGAGGGAGGAAGGCTGT
ATTTTGGCTCATGGTCCGGGGGATATAGACCATTGTGGTGAGAAGGGGTGGTGTCTGGAACTCTACCTGTGATAGATGGGGTGAGGCTTATTCA
CCTCTCAGTGACAGGCCATGCTGCAGAGAGACCTTAGTGGGAACCGGTAAGCCCCGACTTTGGGTTCTCCTCTACCTGCTGGAACCTAGTGCAA
AAGGTTCCCACACCTTCCCCAAACAGTGTCAGCAGCGGGGACTAACTTCTCAAACCCTGAGCCTGGGAGGGAACTGGGCTAGGAAACAGTTTCA
CACTACAGCTGTCCTGGGTTTGTTCCCTTTCTTTATATAAATCAAATCCCACAGGGGTTACGGTTGAGCCATAGTGTTCATATAGTTGTGATTA
GAATACTCCCCTTCCCGCATTGCATTCTATCCATTGATTGGTTTTATTTTAAAACATGAAGCTGAATACATTGGTATATGCTTGTCATCTCAGC
TCAGGAGGCTGAGGCAGGAGGATCAAGAATTCAAAGCTAGACTCGGCTTTAGCAGACTGTCTCCAAAACAACAAAACAATCAAAACCAGGAAGT
TTGCTGAGCCATGCATGCTTCAGGAGGAGGCAGGGCAGAGTGAGTGTGCATGGTCCTTTAGGGCCTTTGCTTAGGATGCTGACCGCTCGCCAGC
CTGTTGATCTGATTGGCACATAGCCTGAGTGTGGTGTGAAGGGAGGGAATGCATGCAGCGGGCAGGCAAATCTTTCAGAGCAGGACCATAAGAC
AAGCCCAGGGATGCTTCATGGTGAACATACCAGGCTGCCTTGCTCCATTGGTGGGCCCCTCACCCATCACGTATGTGCCCAGTGCCCCCTTTGT
ACTCAGTTTCTAACCTGGATCCCTCCCTTTGCCACCCCGGGGTTCAATCTAAGAGTCTGTTGACAGATGGGAAGAATTCCTTTAAAGCAGCGGT
TCTCAATCTGTGGCTGTGGATTGCAATCCCTTCGTGGATCAAAGGACCCTTTCACAGGGGTTGCTTAAGATCATCCTACATGTCAGACATTTAT
GTTAGGACTGCAGTTATGAAGCAACAGTGGAAACAGTTTTATGGTTGGGGGTCACCACAACGTGAGTAACTGTATTAAAGTGTGCAGTGTTAGA
AAGGCTGAGAAGCACTGCTTTAAGTGCTTAATTCTCTGAGCCCCTCCCTCTTTTCCTCCCTTTCCCCCTCCTCCCCTCCCCATCTTCCCCTCCC
TCCTCTCCCCTCTCATGCTGAGGTCAAACCTCATGTGGTTTACCCAGATCCTCCAATGGGATTACTTTGTGTCTCTTTGTGCCACTCCTGGGCT
CACTCCACACTGTATAGCAGTTTTAGACTCACCGCAAAACAGTTCCATGTCCCTGCTCCTCGCTCACCCACCACAAGAGCATGTGTGTTATTTC
ACTTGGTGACCTTACAGCGAGAACATTGTCAAAGTCCACTGGCATGGGAGTCCCTCTTGGTATTGGCGTTCTCTGATTCCAAACAAATGGGTGA
CTGGGTGCTGATATGTGTCTGCTATCAGAGTGTCTGTCACTTCCTGAGCAGTCTAGTCCTCTGCTGCTTCCCCCCCCCCATTACTGTCCCCACA
GGTGTCCTTTCTCAGAATGTCACACAGTGGTAGCCGTGCAATACACAAATTTCCTCAGTGACCGTCTCCACTCTCCTTTCATGTGTGGAGCACG
TGTTGTGTGTTGAGAGTCGCCCAGCAGATTGTGAACTGCCATCCAGCCTTCTAGCCCGTGCTCTGTGCCAGGTTCTCTCTGGTTTGGAAGTTTA
AAGCACTTCTGTGTCTTGGTGAGCATAAATCCCAATGTAGGGGACAGCTCTGCAAATCCGGGTTTGCTTGAGCCATCGCCTGTACGATTGCTTC
CTATTGCTTCCTTACAGCGGATTCCTAGGGGGAGATCTCTCATGCAGAGAGAGCTGGCCCTTGGATTGAGGACTCACCCTGTAGAAACACTCTC
CCACGAATGTCACACTCCAGCCCCTAACATTTGAGTAAAAACTTCTCGCTCTGGGCAGTCTTCAAAGGCTTGAGGAGTTTAAGGTTTTGTGTGT
TTACTAATGGAGATGTACAATGGAAAGGGCTGCCTAGGACTAAGAAAGATCCCGATCCGAGGTCGAGTTCAAGCCGCGAAAGGATGAGAACTCT
GATGATGAGTGATGTCTGCCATGCGTGTCACATTGGAGACTATAGGAGACTATAGGTGCTCATCATCTCTGACACCCAGAGCCCAAGTGTTAAT
AAGATTTTACACTTTTCCACTGAGCATTTTAGTGTTTATCACTTCTTTTATAAAATGTTCAAAATTTTTCTATTGAGTTGAGCATCGTGATAGC
TGGAGGAGAAGTATTTTTCTATTGAGTGTAGTAACTGCTGTGAAGCTCTTCATCCAAGTGCTAAAGGTACGATTTAGGGCTTCACACATGATTG
GCAAAAGGCCTACTTCTGACCTGTATTCTCAGGCCAACTCCAACCCCCCCTTTTTGTTTTGTTTTGTTTTGTTTTGTTTTGTTTTGTTTTGTTT
TTTTCGAGACAGGGTTTCTCTGTATAGCCCTGACTGTCCTGGAACTCACTCTGTAGACCAGGCTGGCCTTGAACTCAGAAATTTGCCTGCCTCT
GCCTCTGCCTCCCAAGTGCTGGGATTAAGGGCGTGCGCCACCACTGCCTGGCTCCCTTTAATGCGTATACTTTTCTTTCTTTCTTTCTTTCTTT
TGGAGACATGATCTTACTAAGTTTCCATGGCTGGCCTTGCACTTGCGACTCTCCTGTCAACCTCCCAAGCAGCTGGGCTTGGTTTATGATACTT
TTAAATGACACTAACATTTAACTAACATTAGAACTACTGTTAATAAAGAAACCCTGTCTCAAAAAAATGTAAGAACTACTGTTAATAAAGTATT
TACTAGGGACTAAACATTGTGTGCGCAAACCTGACCTGCGAAGCAGAAATGTGCAGATACTCAGTCCCTCTAAACAAATGGGAAGCCAAGATGA
CAAGTGGTCATTTGTCAAAGCCTGGGTGTGACCATGCTGGTATTGGTGACAGAATGTATGCTGGTGTAAAACTCCCATAAAATTAACTGGTTTG
TAGTGGCTCACACCTTTAATCCCAGTACTTGGGAGGCAGAGGCAGATGTGAGTTCAAGGCCAGCCTGATTTACATAGTGAATTTCAGGACAGTC
AGGACTATGTAGAAGGATCTTGTCTCTAAAAGCAGGGTGGAGTCCCATGAATTCTGTAACATGTTTTAATAGATTTATCTCACGATGATGCACT
CTAGTAATCCTGGCTTGTGAGGAAGACAGGAGGATTATGAATTAGCCAGACTCTTATCTTAGTGAGTCTTGGAACTCACTATGTAGACTAGGCT
GGTCTAGAACTCAGAGAGTTCTACTTCTGCCTCCCCAGTGCTTGGATTAAAGGTGGGCTCCACTGCACCTGGCTCAGACTCTTAAAAAAGTAGG
TAATAGGTTTTTTTTTTTTTTTTTAATGCCTTTTGCATGCCCTAGGAGCAGCTACCTGCATTTGCAGGCGTATCTTACAGGTAAATTCAAGGAA
TATTGATCATTCTCCCCATAGATAGTGCTTGCTGGGCACATAAGCTCAACCATTCAGGAGCTGGAGAGATGGCTCAGGGGTTAAGAGCATTTGC
TACTCTTACAGAGTATCCAAGTGTGGTTCTTAGGACCCACACTGGGTGACCCACAGCTGCCTGTAACTCCAGTTCCAGGGGATTTGATGCTCTC
TTCTGGCCTCCATGAACACACACATTTACACATACCTTGGTATATGTGTGTACACATCTATACATACATGTGCACACATATACAAATAAAATAT
AATTTTAAATTCAGCTATTCCTTCTAATACTTCCCAAATGTCCCCGCTCCCGCTCCCGTGGTCTATTGATTAACACCATTAATTGATGGGGTAC
AGATTAGGAGGTTGAATCGGGAGGATTGCCACAAGTTTGAGACTACATAGTGAAACCCTGTCTTAAAAGAAAATGACTTTTCAAGAAGGGGAAA
CTGAGGCTTAGAACATAGGTAGAAAATGTCTCAATTTATGGCACAAACAAGACCAAGCGCCCAGGTATACATCTGACCTTGAGTCCTTTAGATA
TTCAAGGGGACAGGCAGGGTGGATACCAAACAGCACTGCAAACTCTCCGGGGGCCAGACTTAAGGAAGCCAAGTGGGCACAGCCTCCTGATGCC
CCAGTCTCCTGGCTGCCAGGCAATCTGGGGTCCTGGCTCTCTGCCAAGGGTGAAAATGAGCTGCAGGCCTCTGGCCACTCTCTGCCCCTCCTCT
TGGGTTTGCATCTCATTGCTGGAGGTTCCACCAGGCTTAATCTCTGCGAGTGCAGACTCGGAGGAGCCAGACTGGAGGGATCAGTGCCAGCAGC
TTGGAGAGCTTCATGAGGCTGCCCTCTGCTTGGGCAAGGAGAACAGAAGAAGTGGCTTTTAGAGCCTCACCTGGCCTGTGCCTTCCCCTGATAT
GGAGTCCGGTGAGGGTCACCAGCCCCAAGCGGTAGTTCACTTAGTCACATCATATTCTGACGCATGTTCCTGTAAGCCTTCCATTGAGATAGGG
TCTATTTCAGAGTAAAGGTTCCTGTGACCCACTTCAGGCTTCAAACGCACAGCACTCAGACAAGTGAGGCTTGGCCATGGCCTCCAGGCAGCTG
GGCTTTTCTGAGAATGGAGAGGCCTGAGTAGATGTCAGGGACAGTGAGAGCAGGGGCACCCTTTTTGTTTTGTTTTCCGGGAAGCTGTCCCATC
ACTAACCATCTAACAACAGGTTATTATAATTCATTGGGTGCCACATGGAAGAGCATAGAAGACAGGGCAGGCTCGTCATATCTGACACTCTGTT
TCCTAAGCATCCTACTTTGTACAAGTTGATTAACTATTCCTTGTCTGAAAACCCAAATCCAATGTATGTCAAAATCTGACATGTTCAACAGATA
ATCAAGTTTTGGAGCTTTGGGATTTGGTAAAGAGTATGCCAGCATCCTAGTGCCAGGACACTGCCAGGTCTGAAACACTTTTGGTGGAACATCT
CAGATAAAAGTCATGTTACCTGAGTGTGATTCTGTAAGAAAAACGTGAAGAACCACATGCAAAATGCACATGTGAAAGCCACAGGCCTCTCTAC
TCGGAGGTGGAGGTGCTGTCTGGTGGCTGTTTGTTTTGGATTGTCTATGTGTCTCTTCTAGTTCCTAGCCATGTGTCTCTTTCCCTCCCATTGG
GTGCCCTTGTCTCTGGGCAGCCAATGAAATACAGCCCTTGTAAAGCCCTGGTACTCAGCTCTCCAAGTCCTCCCCTCAGTCTGTATCCCAATTC
TCCCATAAGATGCTCTGGCAGCTATGATCATTTTTGACTTACCCATGAAGACTCCTTAAGGCTTAGCACACGGCCTTCTGATGTGGCTGCTGAT
GCTGTCATCTACAATGTTCATACCCCAGACCTTGAACAAAGCAGAGTCTGGAGAGATGGCACAGAGGCTCACGCTGCCCTGTCATGGGACTCGC
ATTCACTCCCCACCATCCACACCAGGTGACTCACAAACACCTACAATTCCAGCTGCAAGGGATCTCACAACCTCTCTGATCTCCATGGGCGCCT
CCACTGACATGTACAGACAGACAGACCTAAATCAATAAAAAGCTTTAAACACTATTGTAAGTTTATTTATTCTTATCTGATGTGTATGAGTGTT
TTGCATGCATGTATGTCTGCACACCACATGCATGCCTGGTGCCCACAGAGGCCAGAAGAGGGTGTTGGATTCTCTGGAACTGGAGTTACAGATG
GTTGTGAGCTGCCTGCCAAGTGCCAGGGATCAAAACTGGGTTGTTTGGAQAGAGCAACAAAACTGCTCAGCTATCTCTTCAGCCCCTATTAAAA
ACATGTAAAAAGAAAAGAAAATCTCATGATGTCTTAAGTAAGGTTACGGTTTTGTGTTAGCCATCCTTGCCTGCACGTGAGCGTGTGAACCAAG
GACGTACTGTAAGAATTGGGCTCCCAGCTCTATAATCTGAGTCAGCTGTGCCTGTCACTGCGCTAGCCTCTCAATGTCAGCCTTGTCATCTTTC
TTGACACGCACCTTGCCCTGCCATATGGGGAAGGAGAGTCAAACATAGCCCACAAGGCTTCTCCTTCTGAAGTCTCTTGTACAGCTGAGTGTCT
GTGGAGCCGGTGGGACACAGGCGACACAGTGCATTTTGACCTGGGCCATTTGATCATCAGTTCATTGGGCAGATGCAAGTTCTGCCGGGGTGGG
ATGGGCCCGAGGCCGGGGTGGGATGGGCCCGAGACCAGGGTGAGACACAGCAGATGCTCAGAGTGCAGAACTGAAGGATGTCCACTCCTATGCC
TCACAGACTCCCTGGGAATGGAGGAGGTCCCTTCCATTTCATGCTCCAGTCCTTGTGTGCTTTCCCAGCCTCCAAGCATTTCCTGGCGCCCGTT
ACAGGTGCTGGGACACCAGTGGTAAGCCTGAGAGCCTGGGAAAGCCATTTGTCCCGCCTTCTGGGAGCTTACAGTTGCAAGCTAGCTAAAGGCC
GTTTTATATATATATATATGTATATATAGTGTGGGGGGGGGGGTCCCAGGCATTGTGGCTGGTGGAGTAGACAGGGTCCAGAGAAGGGGAACAC
TACAAAATGCCCCCAAGCTGTGTGGTCCCAGAAAGCCAGCGTTTGTCAGTGATTGACTAAAAAAGAGAAACTCAACAACCTAGTGGAGAGCATT
CTAAGCTGCACTTGTTGGTGCTGCCTGGCTGTGTTTCTGATTAGCTGTCTTTTTTCTTCGTTTCTTATTTTTATTCATTTTACCTTATCTATCT
ATCTATGTATTTTTGAGTCAGGGTCTCACTATGTAATTCTGGCTGTCCTGGAACTTGCCATGTAGACCAGGCTGGCTTCAAACTCACAGAGATC
TGTCTGCCTCTGCCTCCTGAACTCTGGGGTTAAAGGTGTGCACCACTATAAGTGGCTCCTGATCAGCTTTCTATCCTGGGGCTGTAGCCTCTTC
CACATTTATCTCTGTCCCTGCCACCCACCACATCTGATGGAATCTGTCATTGGACACATTTCATTGGAAGGAGAGTGGACTCCTACAAACTGTG
CTCTGACCTCCACACATTCATGACCCCCCCCCCCCCATACACACACACATTAGATAGACTTACTGAGAGAAATGAAAAGGTAGGGAAGTCAGAC
AACCTTTGTAAGACTCTGGGGAGCCTTAGCTTACCAGGGACCCCCTGAGTGAAACTCGTGGAGTCGGGATCGATGCAAAAACCAAGAGAAATTT
ATTGTTCCAGTGCACTGGGGTCATCCCAGACCCAAAGGAGGGGCGGCGACTCCCAGCACCAGTTCTGCTGCGTTTTATATGGTTTCCAGGGGCA
GAATAGAGCATCAGCAACTAGGCACAATATGATTGGTGGAACAGTGCATCCTTTAAACTGATTGGTTTTTAGGGAATAGGTGACAAGGACTTCC
CTTGTCTGAAGGTGAGCAATGGTCAGTCCTGCCGAATGTGTCCCCCACCCGCAGTTCCTGCCCTGTGTGTGGTCTGAGAAATGCTACTAGTAGT
AGCCTCTACCTTCTGGAAGGGCGGAGTGTTTCCCCGACCTTCCCAAAATTCCTGAGTTGACCTTTTCACCTTCATTATCAAGCTTGCCAACCAT
GGCCAAGTACCACAGACTGGCCACATGGCTTTACTTCCCAAGTTCTGAAGGTTAAGAGGTCAAAGGCCAAGGTGCACTCAGCCAGATGAGGGCT
GTCTACATTGCAGATGGGACTTTTCTACTGTATCCTCACAGGCAGAGAGCCCTGTGTCACCTTTCTTTATGAGGTTACCTGACCCTGAACACCT
ATCCAGTCTTTGCCTCCTCTCCAAATAGTGTCATTAGGATTTAGGACTTGAATGTAGGGGAGCGACAAAGGGTTCGGGCTCCTAACACCTGCTC
CATGCCTGCGCAGCGTCATTGAGACTTGAACTCTCAGAGTGAAGGCTGTTCCTAGACGCAGTTGTGGGACTCCTGCCCTTGCAACTGCATCCCA
GTCAGCTTGCGTGACCTTGGGCCAGTCCCTTCTCAGCCTTCAGCCCTTCGCACCCCCTTTCTCCGCGGGTGGCAAGGGAGCCATCGCCAAGGCC
GGGAGGTGGAGCAAAACGCGGCGCTCGCACCCGGGGCGGTGCAGGTGGTGCAGGGCGCGGGCTGCGGTGGCCGAGGTGGGTGTCGGCGGCGGCG
CTGCGGAGGCGGCTGCGGGCCGCGCGCGACCATCCGCGCGTGAACCCGGAAGAAGAGTGTCCCGGGGTCTGGCATGAGGACCGTGGGGAGGGGC
TGCAGCCAGGTTCTGTGGTCAGGGCGGTAAGCATGCTCTGGGGACACTCCCCCCTGGAGAGAGAGGTGGGCTTGGAGTCAGGGCAGGGCTAGCC
CTGGCTGGCACACAACCAGCTCCCCAGCCCCGCCTGTACCTGCTGTGAGCTGCCCAAGGGGCCCGGCTTCTTCCTCCTGGCAGCCCCTCGCTTG
GGGCCTCACAGCCAGCCATGCAGAGCTCATTTCAGCTTTCCACTCCCTCGGGCCCAGTTGGATTTGTGGAGTCCCCTTCCTGCCCACTTTGGCA
GTTTGTCAGGTTCTTGGGGGTTCCTTGTGTCCTTTGGACGTAGTGTCCGGGCTGTGCTTCTCAGGCCTGCCTCCTGGCCTGAGCTGCCCCTGGG
CCTTGACCCCATGAGGCTGGTGGCTCCAGTCGTCCCCAGGACCAGCCCGGGATAGAAATGGCATTTCCTCTTGGCCAGGCTACAGCTGTTTTTG
TTGTCTGAGTTCTCAAGGCTTCCTCTTGGGATGGCAGGGAGGGTAACCAGACTCCTAGGGACCCCTGCTCTCTCTCCCAGCCACTCACCCCTCT
TCCCCTGTCTCTTTCATCTTCTTCCTGTGCTGGTCCCGCCTCCCTTGCCTGTCCCCAGGACAGAATGACTGAGAACATGAAGGAGTGCTTGGCC
CACACCAAGGCGGCTGTAGGGGACATGGTGACAGTAGTGAAGACAGAGGTCTGCTCACCACTCCGGGACCAGGAGTATGGCCAGCCCTGGTGAG
GCCCCTGGGTGGTGACTTGAGGAGGGGAGGTGGGTGGCCATGCTGGGCTGGTTGGGGACCCAGGGGATGTTCTTACTGGATGCCAGCCTTCCTT
GTTGGACAGTTCTGGGGTCAGAGGTGAGCAGGCACTAGACGGATGGGGGCAGAGTTTCCCAGAGCCCAACTCTCAGGAATTCCCGCGCTCCACT
GCTCCTACAGTGAACAGCCAGCCTGTTGGAGTTTTTTTGCCCATCTGCAACTCTCTGCCTCCTGGACAGGTGTCCAGTCTGTCTTCCCCTTGGG
TTGTTTCTGATGGCTTCATTGCTTTCCTTTTTTTTTTTTTGTGGGCCCTTGCCCGGAGCAGCTCTCGGAGACTGGAGCCATCTTCCATGGAAGT
TGAACCCAAGAAACTGAAGGGAAAGCGTGACCTCATTGTGACCAAAAGCTTCCAACAAGTGGACTTCTGGTGTAAGTAGAGCTGAGGCTCTCTG
GGTGGCTCCTCCCTTCACCCCATATCCCATCTGCCCCTGGGTCATTGTGCCAGAGTCGCAGTGAGAGGCATCCACCTGGGGCACACCAGGGCTC
AGCAGCCTGTCTTGCGTCTTAGAGTCCACAGATGACCCCAAACCCAATTTTTTTCAAATTTGAGACAGAAGCCTTGACATTATAGCATGTCCTT
CAGTCCTCCAGGGAGTAAGTATCATGGTTTATTGCTTCTGATGGACCTGGGACTGCAGCTAGCTGGGCAGATCTAGGGTTGGTAGGCTTGAGCT
ACATGCACAGTATTTTCTACTGATCCTCAGTGGCCCCCAGTTGTTTCCCCAGCCAGTAAGGCGGCGGTTCTTATATCCCACCTCTACTGAACTT
TCCCTCTTCTTCACATTCCTCCAGTCTGTGAGTCCTGCCAAGAGTACTTCGTGGATGAATGCCCGAACCACGGTCCCCCCGTGTTTGTGTCTGA
CACGCCAGTGCCTGTGGGCATCCCAGATCGGGCAGCCCTCACCATTCCTCAGGGCATGGAGGTGGTAAAGGATGCTGGCGGGGAGAGCGACGTG
CGGTGTATAAACGAGGTCATCCCCAAGGGCCACATCTTTGGCCCCTATGAGGGGCAGATCTCTACCCAAGACAAGTCAGCTGGCTTCTTCTCAT
GGCTGGTGAGTGTCCCCTGTACCATTCATTCCCTGGGAGAGGTCGACAAGGGACTCGGAGGGAAACAGTCAGGTGATAATTTATGCGTGCACAA
GTGCTTTTCTGTGCACATGTGGATGACGGGTCTTGCTACCGTCTTGTCCAGCCATGTGGCTTTTGTCCTTTGGTGTCCAAAGGCCATGCTGAAG
GGACCCCACCTCAGCAGCAAGCAGGTGAGTCATCACCAGGTAATAGGTAAGGCTGATTTTCCCAAGTGTCCTCCTGTGTGGGTGTGTATGCATG
TGGCTGTCCATTCAGTGGTATTTACAAAGACCTCAGCCAACCCGTCTTCTAAAATGGAAGACCTATAGCTCGGGGCTGCAAGGTCATAGAACAT
GACGTTCTGCTCAGAAAATGGGCAGGCAAGGGACTGGACGGAAGGCCTTCCAAGACAGGCACGGTTCCCATTGTTGAGAGTGAGCAGCGAACAA
GCATGTAGATGTGATTGTTGCCACCAGAGGGGGAGCACAGCATACTTTGGGACATTATGTTTGTGGTACCTGGAGCCTGCATTGGGAATGTCAG
CCAGCTTTACTGTCTACAGTCTAATGGCCTTGAGTGTATCACTTTTTTCTCTGGGCCTGTTTTGCGACACAGGCTAATAAGAGCTGCGGTGAAG
ATAACTCCAGGTGGCCTGGCACCGTTCTTGGGAATGTGATTTCTCAGTCACTAAAGCATTTATCATTGCTTGATGTTACTGCTGTTGGCCTGGT
TGAAGGCTGAGTTCCTTCAGAATAAGAATTGGAGAATGAGGTGCAGCTGGTCTTGGGAGGGGATAGACCCTCATTCCATGTAGACAGGAGAGAT
GGAGAAGAAGTCGGCTGTGGTAGCAATGGAGATCAGCAGAGAGGATGGCGGGGACTGTGCGTGATTAATTTCCCAAGACTGAGGACTCAGGGAA
GCTGGTGATGTGGCCAAGCAGAAATGTGGCTGAGTCAGGCTGTGGCACAGAAAGTCTTGATCTCGGGGAGGAGGTTGGGGAGAAGGTTCATGTC
AGATGAGCATGGTGATGGCTTGAATTGAAGTCTGGAGGTGGGTGACTCTGAGAGATTCAGGAGGGAGAGCCCTGAGGCTGGTGGGACAGCTAGG
GTGCCAAAGTGAGGAGTAGGGGCTTTCGAAAACATAAGGAGGGAGTCCTAAGACTGCTCACGGGGGAGAGATGGACAGTTGGTGGGGCAGTGGA
GTGGTAAAGGAAAGCAGCGGGACCATTTATACCACATGTATGCCTCTCCCTGAGCATCTCCTTGGTGCCAGGCCCGGGGAGCAGGCCAGATCAG
GTGTAAATCCTACCTTCATGGGCAGGAGATGGAGCTAACCAAAGAATAATGAAAATGCACATAAAACCACGGGCAGGGGTTGATGAGATAGGAA
GCTAGGAGGAAGTGAAGAACCAGGGGAATCCATGTGAACAGAGGACAGAAGAGGCCGACAGATGCTGAGCTGGGGTTGAAGGAAGAACATGGTC
CTCTGTATTAGTTAGGGTTCTCCACAATAACTGAATCCATCCATCCATCCATCCATCCATCCATCCATCCGTCTATCTATCTATCTATCTATCT
ATCTATCTATCTCTCTATCTATCTATCTATCTATCTTTCTATGATTTATTAGACTGACTGACTATCTATCTATCTATCTATCTATCTATCTATC
TATCTATCTACCTACCTACCTACATACCTACCTATCTACGATTTATTAGAACGACCTACAGAATGTGGTCCAGCTAATCCAACAGTGGCTGTGT
GTGAATGGAAGGAAGGTCCCATGAATCAAGCAGTTGTTCATTCCATGAGGCTGGATGTCTCAGCTGGTCTTCAGGATACACGAGAATCTTGAAG
TAGCAGGCTCCAATGCCAGTGAAGGAATGAACTTGATAGCAAGGCAAGAGCAAGCAGGCAAAGAACAAAAATCTCCCTACTTCCATATCCTTCT
ATAGGCTTCTGGCCCCAGATTAGAGGTGTGACTTCCCACCTCAAAGATCCAGATTAGAAGTGGATCCCCAATCCATCCCATAATTAGCCTCCAA
GCAATGGCACCATTGCATACACTAGCAAGCCTTTGTTGCAAGGACGGTGATATAGCTGTCCCTTGTGAGACTAGGCCAGGGCCTAGCAAACACA
GAAGTGGATACTCACAGTCAACTATTGGATGGATCACAGGGCCCCCAATGGAGGAGCTAGAGAAAGTATCCAAGGAGCTAAAGAGATCTGCAAC
CCTATCGGAGGAACAACATTATGAACTAACCAGTACCCCAGAGCTCTTGACTCTAGCTGCATATGTATCAAAAGATGACCTAGTCGGCCATCAC
TGGAAAGAGAGGCCCATTGGTCAGGCAAACGTTATATGCCCCAGTACAGGGGAATGCCAGGGCCAAAAAATGAGAATGGGTTGGTAGGGGAGTG
GGGGGGACGACTTTTGGGATAGCATTGGAAATGTAATTGAAGAAAATACGTAATAAAAAAAAAAAAGAAAAAAAAAGCAGAGAGAATATCCAAA
GTAACAAAGTTAAAATTGAATGGTGGTGGTGGGGGGGAGGGGCACAACGACAGATATCAAGGAATTGTGATGACACACTTTATTCAATAAAATT
TGAAAAAAAAAAAAAGAAGTGGATCCCCACGTCAAGTTAAGCAAAAAAAAAAAAAAAAAAAAACAAAACAAAACAAAACAAAAAAAAAAAAACA
AAAAACAAAAAACAAAAAAAAACAAAATCCTTTGCAGGAGTCCCCCTCAGTTCTTGGGTTTTACTTAATTCCAGATGCAGTCAAGTTGACAATT
AAGAATAGCCATCACATCACCAAAGCACACATTGGTGAGGGGGTATCAGATCAGTAGATCTGCTTGAGCAGAGGCCCTGTGGTGGGAAGGGGCA
CACAGCATATGTGAACAGCAAACGTGGGGCTGGAGAAGGCCTGGAGGTGGGGACTGGGAGGGGTCTTCTGCAGACTTTACTCTGTGAAGGATGC
TGGGAAGTTTCCAGGTAATTGTTTCAAGTAATGATGAATGACCAGATAACTTTTGTTTCTTTATTTATTTGCTTGGCTGTGTGTATATGTGTTC
AGGGTGGTGATGTGTGTGTGTGTGTGTTTATGTGTGTGTCTAATTATTCTCTCCCTTATGTTTGAGACAGGGTCTCTCTCTAAACCTGGAGCTC
ACCAATTCAGCTAGACTGGTTGACCAATAAACCTCCAGGGATCCCCGTACCTCCCCAGCACTGGGATAGTATGTGCACGCCACGGCATTGGGCT
TTTACCTGCGATGTGGGGATCAGCTCAGTTCTTCACGATTACACAGCAAACACTGTCCACTGAGTCATGTCCCCAGCCCCACATTAATGTCTTG
AAAGGACAACATTGGCTGCAGCCTTTAGAGCTGACTGGGGCAGAGATGTGGCACTGGTTAGGAAGCCGGTAGTCAATGTAATTCCAAACCTTCA
ATGGAGGTGGCGTGGGTGGAGACAGGGACAGAGAGACAGAGACTGAAGGATCGACTTGAGGACGAGATGGGGGATGAAGGCCAGTCAGGATGGC
ATGGCTTTAGTAAGGGACCCAAGTATCAAGGCCAGCTTAGGTCCTCCCTGCCTCCCACCGAGCACTTCTGAAATGAGGATTGCTGGATGGAGAG
AAGGTGGAGAGTGACTGGGATTCTGGTGTTGGCCTTGCTGAATGGCGATGTCAGCAGACAGGTGGATGTACCGTCCTGGCCCATGCAGCAGGAG
TGTAGAGATGTGACTTATGTTCGTAGGTGGCTAGTGGAAGTGGATGGGGCCTAGCCTCAATGTCAAGGGACTTCAAAAGTTAACAGTCCAGAAG
GAAGCATGGGAGAGAGCGGCACTGGGCGAAGGCAGGCCAGGAGAGCGATCTGCCCACATAGCAAGAAGAGAGGTGTACAAAGGCTCTAGCCTCT
CATGCTAATGGGGAGCCCAGGGTGACAGAGCTCAGAAGTGTCTATTGATATTGTAGCCACAAAAGCATTGTGACATCAGAGGAACGGTAGGTCC
AACCTGGGATCTTGAGCTCCTATGGTCTTTGGCCATGCTGACAGCTCAGGGTGGGGGCATGCTACTGGTACCTAAAGACAGAGGCTGGGGGCCA
GGTTTAAGTGGTTTGTACTGCCTAGAACTGCAGCCTACAACAAAGGCTTATCAGGAGCCCCTTGCAGGAGAGTACAGGAGCTGGGCACCTGATT
AGCATAGAGGTGGCCTGTAGAACAACTTCTCAGAATTTGGCTAGAGCTCAGAAGTGAAAAAGAGAGATAAGAGAGGTGGATGGTGATTAGAGGG
TAAGGGGAGCTGGTACTAGCAAGAAAGCATTCTTAGGGCCAGCAGGAAGAGGCTTTGGAAAGGAGCAGGAGGATGAGGTGAGAACACCATCCAT
ACATGCATTTGTAGCACCTCTGTCTGGAAGGTTCTGCAGAAGCCGCTGAGTATGGAAAGGGGCGGCTCAGCACATTTTCCATCACCTAAGGGAC
AACCCAGGGTAGGTGGGTGTGGTAGACTAATTAATTGCTCTTTGAAGATTAATAGTGTTCATTGCCAAGTTGACAAGATCTGGAGTCACCTAGG
AAACAAGTTTCCAGGAGATTGTTGAGCCTGTGGGGAGAATTGTCTTGATAGGTTGATGGATGTGGGAAGACCCATGGGTGGTGGCATTCCCTAG
ACAGGGATGCTTGACTGTATACAGTGGAGTGAGGTGAGCATAGGCACTCATCCCTCTTTCTTCTGGATTGCAGGTGTAATGCAACCAGCTACCT
AGTGCTCCTGCCCATGTGGCTTCCCTGTTATGATAGACACTGGGGCTATGAGCCAAATCACCCTTTCTTCTTCTTTTTTTTTTTTTTAATTTTT
TAAAAGACCATTTTATTTTATTTATATGTATACACTGTCACTGTCTTCAGACACCCTAGAAGAGGGCATCAGATCTCATTACAGATGGTTATGA
GCAACCATGTGGTTGCTGAGATTTGAACTCAGGACCTCTAGAAGAGCAGTCAGTGCTCTTAACCGCTGAGCCATCTCTCCAGCCCCCACCCTTT
CTTCTTAAGTTGGGTTTGTCAGTATTTTATGGCAGGAATGAGATTAAGATAGGTCCCACAGTGTCCCCATTCTTGTCCTCAGAGCTGGTGACTG
TCTTTCGGTGGCAGAAGGTGCTAGCATCTGTGGTTACCATGAACATCTTGAGTCAGGGAGATGATCTTAGGTTTTCTGGCTGGGTTCAAGATAA
TTACAAAATTCTTCCATGGAGGAAGCATCAGAGTCAAATCAGATGTGGGGAAAATGGTTTCTGTTGACCTCCCAGAAATGGAAGGGACTGGGAA
GTGTTAGGTCTCAAAGCCTCTCTCTTCCTCTCTGGCTCTCTTCCCCATCCTTTCTCATGGTTGGCTTCAGTCTGCACCCTTCCGGATGCCCCTG
CCTGTGCTCTCCCTTGTATGTATAATAAAGACCTCAGCCCTTTAGGAGCACCCTGTCCTCTCTGGTCTTCTCATCCAGGAGGCAGGCATGCTGG
CTCCCTATTGACGTGACAAGACAGGTTCTTGATCAGAAGAGGTCTGACCTGCAGAATTGTAAAAGAATACATTGGCTTTGTTCAGAGAGAGAGA
GAGGGAGAGAGGGAGAAGGAGAGTTAGTTCAGCAATAGGAGACTTAGACTAACAGAGGAGGCTTGTGGGTCAGGGCTATCTGCTACAATCAGTG
TCTTCTTCATTTGCTCCCACTACATTGATCAAGGCAGTACCTTTTGCTGACTCTGTAGCCAGCTTTTGCCTGGATTTCCCTGGCTCGCCTTCCT
GAGTACTGCCACACCTGCCCAGCTTTCACAGAGTCTGGGGACATGAACTCCACCTGTGCAGCAAGCACTTTATCCACCAAGCCCCAGTAATCTC
TTTATTTTCCCCGTGTTTGTCCCTGACTTTGATTACTGTTACCACCACAGGGAGCTTTGCCAGAAGGAAGATTTTATTTACTTAGACAATGAGT
ATCTGTCACTGTCTAGTGACAAGTAGTGATAACTGATATTGCATAGTCAACTCTTGTGTGACACTACCTACCTCAATGTCAGGAAACACAGCCA
ATCGCTATTTTCTGTAATGGGCAAGATGAGAAACTATCCCGTTTTGTGGATCGCAGTGTCTCTTGCAAGGACAGATGTCCTTGAGATGGTGCTA
AACCCAGAATGTACGCAGGTCACCTCCTCTCCCCAGCATCCCAGCTTTGTGTGTGACACAGTGCTCTGTGCAGGATCAGTTGTCCCATGTGACT
GCAGGGGTGCCAGGATGTGACTCGGTGCTGTTGGAAACCCTTCTAAAGCACGGTATGGTACTTCGCATCCTCATCCATTGTTTGATTTTTTCCC
CTGGCCATTTCAGCTTGCACTGGTGTCACCTGCAGGGCAGGCAGGTCCTTCTAGGTTTGCTCAAGGTTCTTCCTGCTCCAAGCCATCCTTGTCA
GTCCCACCTCCTACACTTCCAAAGGTCTACTGTAGGACCTGATGATGCCCTTTGCTTCCCTGGGAGAGCCTACTCCTCACCCTGAGGAAGGGGT
AGGTGGTTAGTACTGATACTGGGTCCGTGGGATCAGGTGCCCCTCCCAGGCAGAACCATAGGACCCTTAAGGGGGAGCACTGTCTGACATGGCC
ACAGGGCTCTGGACAGTTCAGATCCTGGTTCTTCAACTGGCTGGAAGTACGGCTTTGAGTTTGTCATCTGTTCTCTCAGTGGTCTAGCTTCAAC
GTGTGACACAAGCACACAGCCTCTGGAAGACAGAAACCCAGGGAGCTAGAAGTATTGATGTTCAGTGAGCCTGGCGCCTGTGGTGATGGGCAGG
TGACAGGCTGCTTTGATTGTCCTTGGCTAACTTCATGTGGCTTCAGGAAGAGAGAGAGAAGATGAAGTATGTAAGATTGGATATAAGGTCTGGG
TTTGTGTGAACAGGCCTCATTTAACACATCTCCAGGGCCACACACACACAGACACACACACACACACACACACATGCAATATTTTATATGTGTG
TGTGTGTGTGCGTTTATGGCAATGAATACACAGAAGGAAAGAAAATCCACCCCACAGCAGTCCAACAGTTCTAGGAAGCATCAGAAATCACCCA
ATGCTAAGAGGCGGAGGCAGAGAGTCTGGCAGTTGCCGAGGGCTGAGTTACAGGGAAGCAAAGCACCAAGTGTCTCTCATGTAAGACGACAGTC
CTCGAGGTCACTGTAGAGCTCAGGGCCTGGAACAGTGTTGTGTCAGGGCTGCCAAGGGCAGTGTGGATAATTCATGGAGTCACAGGAACAATGG
TCTAAGGCAGAGGTAGGTCGGGCATGGGTTGGGCTGATGTATCAAGGTGTGCACTTCTTCCCAGACTCCTCTAGGGCAGACTGTTACAAGTGAA
CTCCCAGTGGGTGTGACAGACCCCTGGAAGTTGACTTTACAAACACAAATTTTATCAACAGCAGTCTTAGAACAGTAAAAGGAAGTCCTCATGG
CTTCATTGGAACGATGGTAATAGACTAAACCTCTGAAACTGTAAGCAAGCCCCAATTAAATGGTTTTTTCATAAGATTTGCCTTGGTCGTGGTA
TCTCTTTACAGCAATAGAACAGAGACTAAGACGGTAGTTGGTACCGAGGCCTAGGGTGTCGGTGTGACAGGCACGACCATATTGTTTGCTGGAG
GATATAGAAGACTTTGGGCCTTTGGGCTAGGAGAGCGTTTGGATGCTTTACGTGGGGCTTACTGGGCCACTGTGAGGGCCTAGATCAAGAAGTT
TTATAGGGAAAGCATATTACCAAGTGTCCTAGAAACCTTTCCTGTAAGATGTTGGCAAAGATTGTAGCTGCTGTTATCATTGTCCTAAAAATCC
GCCTAAGGTTAAGTTGAAGAGTTTTGGATTAATGGCACTGGCAGAGGAGATTGTGAATCTTCACTGTATGTATGAGTTATTAGTAATCACTCTG
GCAGACCCATAAGGAGAAGGAGCAAACTGTACAACCAGAAATCCAAAATATGCAGTTTGAGGAGAGAGGGAGCAGCAGGAAACGTGATACTGGA
GCCAGGATCTGTGCTCAGGGGGAACAATGTGTAAAGAAAAACCCGATGCTAAAAGTAATAAAGGGCTTGGTGACCTGAGGATAGGACCCCATCC
AGCCAAGGTCCTAACGGATGAGAAGGAACCAGTGGCAAGCGTCAACAGTGAAGGGAAGCATCCACAACAGAGCTGATACACTGCCATTCTGAGA
AGGGCCAAGTGCTTGCCCCAGCAAGCAGCAGAACTTGGCAGCTTTGGCTACATAGTTCTGGCTTTAGAGTTCAGGATACAAGAAAGGGGTTGTA
GAATCTCCTTTCATGGCTACGGAAAGTTGCCAAGGCCAGGCATATGTGAGGGGTATCCCTACATGGAGGCCTAGAGAGACCATTGTGTGTAGCT
GTGAAGGTGAAGCCTGGGTTTGCCTTGGAGATCCCCAAGGTGTTGGTTATGTCAGAGTCCTGGGAGACTTGCAGAGGAGAGCTGCTAACCTCTT
AGGTTGGGACCCAGCCTAAGAGAAGTGTGTTGAGGTGGACAAAGCTGAAGGGATTTAGAGATCTGAAGACCATTTTGACCTCAGCCAAGGAGAT
GCAGAATTTGGAGTTCATTCTGCTGAGCTGTGGTCCTGTTTTGGCCCAGTATTTCCCCCCCACCCCAACTGCTTCCTTGGCTCCCTTTTGCAAT
GATAATGTATGTTATATGCCATTGTATGCTGGAAGTAGGTGATCTGCTTTTGATTTTGGTTTTACAGGGGGTTACAGTTAAGAGATGGCATGAA
TCTCAGAAGAGACTTTGTACTTTTAAAAGAGTGTTAAGACTGTGATAGACTATGGGAATTTTTGAAGTTGGACTGAGTGCATTTTTGTATTAAG
ATATGGCTATAAACCTATGGGGGCCCAGGGAGTGGAATGTGGTGGTTTGAATAAGAATGGCCCCAGGATCATATATTTGAATGCATAGTCACCA
GGAAGTGGCACCATTTGAAAGGACAGGAGAGATTAGGAAGTGTGGCTTGTTGGAGAAAGGGGGTGGGCTTTGAGGTTTCAAAAGCCCTGCCAAG
CCCAGCATGGCTCTCTCTGTCTGCCTATGGATTAGGATATAGCTCTCAGCTACAGCATCCCCTGTCAGCATGCCACCATGCCTCCCACCTTGAT
GATGATGTGTTAAACCTCTGAAGCCATAAGCCCTCAGTTAAATGCTTTCTTTCACAAGAGTTGCCTAGATTGTGGTGTCTCTTCAAAGCAGTAG
AACAGGGACTAAGATTTCTAAGTCTTCACCATTTTGTCCCTAAAGCTTGTCACTCTAGCTGCTCCTCTGATGGCATGTACTGAACAAGCTTGAA
TAAAGAAAATCCTGGGGCTGGAGAGACGGCTCAGGGGTTAAGAGCACTGACTGCTCTTCCAGAGGACCTGAGTTCAATTCCCAGCAACCACATG
GTGGCTCACAGCCATCTGTAATGGGATCCGATGCCCTCTTCTGGTGTGTATGAAGACAGCAACAGTGTAGTCATATAAATAAAATAAATCTTTA
AAAAGAAAGAAAGAAAATCCTAAATGGCAGTAAAGCTAAGCCAGGAACGTCCCCCAGGAAGCCTTTTGGCCTGCGCCAGCCACCATAGAGGCCA
CAGAGGCTTTGGGTTGCTTCCGTGTCCTTCCCAGCACAGTTAGATCCCTCCGCTCCACTGAAGCTCTAAGGAACCAAAAGGTAATTAGTACCAC
TCTGTGTTGACTCGCATACACCACGCCTCAACCTAGAATTAACAACAGGGAATTTTCCTCAATGGCAGTCATAGTCATGATGCTAATACCCACC
ATTAGCCTTTTGCTATGCTCAAGGCTGCAAGGCTGCAAGGCTGCAAGGTGGTGTTACCGAGACAACTTGCCGGAGGCCACACATGCATGTCCTA
AACTGTGGAGGTGTGACATAGGTGTGCAGTGTTGGTGTGAGGACGGAACTCCCGGGTATACTAGGGCATCTGTCACCTGGGAACTGGTGGAGGA
GGGGGCCTGAGCTGCAACACATATGACCTGAATATCTTAAACATGTGTACTTGTGTGCACATTTGAATTATTCATTTACTTTATGTATATGAGT
ATACCGTAGCTGTCTTCATACAGAAGAGGGCAGTGGATCCCTTACAGATTTTGTGATTGCTGGGAATTGAACTCAAGACCTTAGGAAGAATAAT
CAGAGCTCTTAACCACTGAGCCATCTCTCCAGCCCTCTTATCTCTGTTTTGAAACAAGGTTTCTCACTGACCTGGGTGTGCAGATTGGCTAGCC
TGGCTGGCCAGCAAGCCCCAGAGATCTTCTGTCCATTTCCACAGCATTAGGATTACAAGGTTATATCACGATGTCCAGCTCTTTATTTTGGGTT
CTGAATGGGAGAGAAAGAAAAAAAAAAAGAAGGAAGGAAGGAAGGAAGGAAGGAAGGAAGGAAGGAAGGAAGGAAGGAAGGAAAGAGGAAAGGG
TGTGTGCGCCTGGGAATGGTAGAGGAGAAAATGTAGATAAAAGGGAGAGCAGAGGCAGGGACATCCAGGAGAGTCCAGAGTGGGTATTGCCAGA
CTGAGCTGGATCATGAGGGGAGAGGGAAGAGGAGAGCCAGGTACAACAGCTAGGAGATCCAAAAGAGGAGAGGTAAATTAAAATGGCTGGATTA
TACAAGGAAGGACAACCCAGCCCACGAGCTGGAGAACTCATGGGAGAGGATGGGGTATGCCAGCCATACCCTGTACCAGGTAGGGACTGAGGGA
TGCTGGGAGAGCCCAACTGCCAGGTTAGCATGGATATGTTAAGCAGGCACCTCAGCAGCTTGTCCAAGGGTTGGAGACTAGCAGAACTCAGGTC
TTCACGCTTGAATGGCAAACATGATACCAATGAGGTCATCTTCCCAGCACCCTGTGACTGAGAAGTATAAAGCACAAATGCAGATCAGGCTCAT
ACAGATAAGTGAGGAGCACCCCACTTCTCTCTCCTCCTCTAGCAATAAAGCGGAGTTGAATGTTGGCAGAGTGCAGCCTGGGTCCCCATAGCTC
TGTGCTGGTGACCACCTGCTGGTAATAGTGGAGCATACATGCTGGATTGTGGAGTGTCAATGCTCTTGATACACCTGGCTCACAGCTCCTTTGC
CCGTAGCTGTGACAGAATACGTGAGCCAGTCAAGGTAAGACCTGGGCTCACAGTTCCAGTGGTTTCAGTCCCTGACTCTGACTGCTTTGCTTTG
TGGTTGCTGTGAGGCAGTACATTATGCCAGGGAGCACCAGGAAGCAAGTTGCTACACCATGTGGCCCCTAGAAAGAGACTGGCCTTCCATAAGC
ACCTTGAAGGTCATGTCTTTGTGACCCAAGACCTCCCAGTAGGCCCTACCTCATAAAGGTTCCATCACTTTCCAATTACACTGAGGGCCAAGTT
ACTAACTCAGGGTCTTTGGGGGACAATCCAGGTCTAATATACACAGATGTAATGCATGCAAGCTGCACAGCCATTACCAGCAGGGGGTCACACA
CCAGCACCCACCACACACCCGTGCTAATAGGACCGGAGGATCTGTGTGGCACTCCGCAGTGTGGTAAGGCAGTGAAGAGTCCAAAGTTCTGAGG
GGTTTGGTTTTGGTTTTGGTTTTATGTTTGCTTGTTTGGTTGGTTTTTGGTTTTGTCTTCTTCTTCTTCTTTTTTTTTTTTCAAGACAGGGTTT
CTCTGTATAACAGCCATGGCTGTCCTAGAACTCCCTCTGTAGACCAGGCTAGCTTTGATCTCACAGTCTGCTTCTGCCTCCAGAGCGCCATCAT
GCCTGGCTCTGAGTGGTACCTCTTAAAAATGTGAACTGTGTTGCCTTGTAAGGTATAAGAGGGTTGTGTCCCACAGCTGGTTGAGGGAAGCCTA
GCAAATTAATTGTTGGCCCCAGTTGGTACAGGCTGTCCTGGTGTACCCACTCATCCCTTCTTTTCTAACATGTCAACACTGTCACGTTGTGGGT
GGGTTTTTGCCCTTGCTTTTCACTTAAAAGGCTCTTTGTTCCATCACCCTCCAATCAACCAGGATTCTACCATGTATATACACTAAGAAAGGCA
GTGGGGGTGCTACCGGGGATGGCAGAGCCCTGTGTCTTGTCTCCACTCCTTAGACCTGGTGAGGGAGCCTCTCGAGACTCGGAGCTAACCAACA
CACCATCAACAGAAATGTTCTTTATCACTAGGGTCTGGAGAGAAGTAATGGGTGTCATGGGAGTAATCCTGAGAGTCCAGGGCTGTGGCTCAAA
CCAGGAGCTGCAGAGGTGGCCTAGTCTCAGTCCCACAGATTAGAAGGGAACAGGTCCCCACAGCTCGTCAAGGAAATGCTTACAGCAGGATGTG
TGGTCGCTTGGAGGCCACTTCAGTGAGCATTAGTTACTACTGCATGTCTGTAACTAAATGCCCAGTAGAAAAGAGGAAAGGAAGAAATGCTGAT
TTTAGCTCAGGGTTTCAGAGGGATTTCAGAGCATCGTAGCAAGGAAGTGCTGCAGAGTGTATGGTCTTGAAAATGTCTCACGTCTAAAGCACAG
GGAGGGGCAGGAACCAGAGTCTGTCACCTGGGCCCCTAAAGTCTTCAAATATAGTGGCACCAGCAGGGGACCAACCACACTGTCAAAGCACAAG
CCTGCAGGGAACAGTCAAGACTAAACTGTAATATTTTCTAGAGTTTTGCAAAAGATCTAGTATTGCATATTGTTCTTAGGAGAGAAAAATGTAC
GTCTCAAAAATGGGGTGGGGGAATCTTCCGGCTCCAGGATGTGCTGAGTATGTCCTGTCAGGGAAAAAGTGGAAAGGAGTTGGAGGCTGAGAGG
GTCTTTATTTGGAATTTGTAATGTTTATTTATTTGGAGTGTGTGTGTGTGTGTGTGTGTGTGTGTGTGTGTGTGTGTGTGTGTTGGTTAGAGGA
CAGCTTTCAGGAACTGGTTCTTTCCTATCTGCATCTGTGATCTGGGGATTAAACTCAGTTTGTCAGGCCCAAGTCTTAGCGCCTATACCCACTG
AGTCATCTTCCTGTCTCAACTAGCAATTTTGGGGGAGCAAAGGGAAAGATCTGAGCTGGAATTGACTGGTGTGCATTTTGTCGGCGCTTGCGGT
CAGCTACGTTATCCTCTTATAATTTCTGCGTATTTGAAATGCTTCATGAGTGTGATGCACTTGAGCTATGGAAAGGAGGTTCTAGGCGTCGTCC
CAAGCTGGGCTGATCGCCCCTCAGAGCAGGCGACAGTGACTGAGGGACACAGAGGCTGCATTATACTGACTTTTTTCTTTACTATGACAAATGC
CTGACATAAATGCTAAACAAAGGAAAGGATATTTTGGCTCATGGTCTAGAAGGTTCCGGTCCATAGTCTTTGGTTCCATTAATGCTGGGCCTGT
GGTGAGGCAGAACACGGCAGTAGGAGCCTGTGGAGGAGGGTGTTCACCTCACAGTGGACAATCAGGCAGAGAGCAAGGAAAGGACTCTTCACAG
CATTCTCTAAGTAGAATTCAAAACGGAATCTCCTGTTGTTCTTGGGAGAGGAAAACGCATGTTTCAAAAATGGGGTGTTCTAAAATAAGCATCG
TCATCCGGAAAAGCATGCGCTCAACACAGACACAGGAGCCTGATGTCCACAGTTCATGTTCAGACCATAACAGGCCAGAGTCAGTCTGACAGGT
GGGCAGGGCTCAGGCGTGCAAACCTGACCAGAAATGAATCTCATCAGAAGCTCAATGGATAAAACCTTGAGATCACATGAGTCACATGGTCTGA
TTACAATTGACAACAGTTTGTTCTGCCATTGTTGAGGAGAATGGCGGAATGCTAGCGCCAGTGTCAAGGGGCTTGTGATTGCTCTGCTCCACCA
AGTCCTCAGGGAACCAGCCTCGATCTAAAAAGTTTCCACAATCAAGTGTGTGTTGTATGCAATTTATACCTCAACTAAGCTGTGGAAACAAAGA
GGAAGGATCATGTCACTGTGAAACCCAAGGAGACAAATGAAAAAGAGACAGATCTTCAAAAAGAGTCAGTAACACTGAAAGTGAAAAATATCAA
TTGAATGAAAGATACTCATTACAAAGGAAGCAAAGCCTTGGGCACAGTGGTGAGTGCCTGCAGTTCTGCACCCAGGAGGATGTGTGGGAGGATT
GAAAGTTCAAGGCCAGCCTAGGTACAGAGTGAAGGGCTGCCCCCAAAATTTTACAAACCGAAACGAAAACAAAACATCAGAAAAGCAAGAGAAG
TAAGAAAGGAAAAAACCCCGATGGATGGAAGAGACTGGAGTAGGCAGAGAAAGCACTGGTGGCCTGAAAGAGAGCAGAGAAATGTTCCCTGACA
CGGCTCCGAACGACAGAGGAAACTATGGAGAAGCAGCTAAAAGGCATGGGGGCTGAATGAGCCATTCTGTTTATTTAATAGAAATTCTACCGCA
GGAGAGTGGTGTGAAGGAGAGGTTGTACCGAAAAACACAAAAGCCGGGGATTTCCAGGAAGTAAAATGTGGACGTGCTCATTAAGGAGTGAAGA
GAACTTTATAATCTTTGCTTCACAAATAATGCAAAATGATGCCAAGTACACAAATCGAAAGGCTATACACTGATCACCTACAAAAGATCCATAG
TAGACTAACAGTAGGTTGCTTATAACTTGGGTCAGGAAAGAACTAGAGACACCATTAGATAAGCTGACACCCGTGCTCTCTGATGAATATTCCT
TTCTGAGTGTATCTCTCTTCCTGCTTTAGACTTCAAGCTTAAAATCTATATCAGAAATGCCTTTTAAGGGCTCCTGCCCTCCACAAAGAAAGTA
AGGGACAACCTAGAATTCCATGGATCTCGATACTCAAAATGATAGTAGAGTATAGACATTTGGACACAACCCCACCCCAAACCCCAAGGGTCTT
GAGCATTTGAGATTTCAGCAGAGGATGAACCCTTTCAGGTCACTCGTGCCTGGGCCCCAGTGACGATCTTCACCTCCTACACACGCAGCAGAGA
AAGAGGCTGCCTTCAGCTTTTGTCACAACAGCCTGGCACCTGTATGGAGTGGCAACCCTGGGCCAGGTTACCCAGGATCCTGATTCAGTCAGCA
CGGATGAGAGTGGCGTCCCTTCTCAGGTCGTCTCCAGGGTCTCTAAGACAATGGGAATGGGTGGAGAGGAGACAGCGAAACTTAGACAGCGGCT
GAGGTTGTGGTGGGGGCCATGTAGGAGGGCAGAGGGAAGAGGTGAGATGGGAGGATGGAGCTGGCTCCTGGGCCTCAGGTAGGGAGCCCAGGCA
CGTGCGAGCCCCTTCTCTAATGGCCCACTTCAGACAGTCTCTCTGTTTTCCAGATTGTGGACAAGAACAACCGCTACAAGTCCATAGATGGGTC
GGATGAGACCAAGGCCAACTGGATGAGGTGAGTCCCATCCCTGCCAGCTCCAGTGAGCCTCACCAGCCTCTGGGCTGGGCCCAGGAAGTATCTT
GGGCCTCCAGGATCTCAGAGCTCTCCAGGCTGCTCCAGAACTGCAGCTGACAGGGTTTGAAAGAAGTGTTCTGTTCCCCAGGCTTCCATCAGAG
GCTCTAAAGAGCCTGTGGCCAGACCAGCAGGCTGCAGGGATCGGGCTGCATGCCATGACTCCCAGTAGAATCCTGCAAAGAGGAAAATGTCCCC
AACCTAGGTTGGACCACTAACATTTATCCTAGGGCAGATTCTTGTTGTCTGGATGACATCATTCCAGGGAAGACCCAGAAGTCATCCTATGGTT
TTATGGGAATGAAGGTTTCTAACATCTCCAGCTCCCTTCCCTATGGGGCTCACATTAAACTTGACTGCTGACCAGCATGGGTCTACCCTGGCCA
AGGCTGTGGCCTAGGACTGTGTGTCAGAAGCATCCTTAAAGTCTAGCCAGAGAGAGCAGAGAATTGAACAAGGGTCACTTCCCAGGTGGCCACA
ATGAGTTGGCATCCATGCTCAGAGCTGGCCTGTGTGGTATGTGTGGGTCCTACCAGTGGGTGTGTATTAGGAAAAAAGTCAGTTTAACTCCAGT
ACCACTTGGTGGCCTGGATCCATGGGCAACGATGGAGCATTTAGATTTGTTTAGAAGAAAGGAAGACCTTTGAGGACCAGAGACCCAGCTACAT
CATGAATCGAGGCCCTATCATGTATAGTTCTGTTCATCTCAGATAGCAGATGGGTGACCTTTGGGAACCAATGGGTAGGTGAGAGATGGGGGCA
GCAAGAGATGCCTCAGGATGTCTGAGCACATGAAGAGGACCTGGTTGGAAGTGGAAAGTCATGAGGAGGTCCCCTGTGTCCTTCTCCGTGGCTT
CACTGACACCTGTATGACCTCAGGGCTGCGGCTGGTTTGCAGGACAGTTGGGCTCATCCTCTTATTGAGAGTGCAGCTGTTTCCACTGCGGCAG
GCAGGTGCAGAATCAGCGCCCACATGTGGCCTCCTCCTCTACAAGATGGTCTGCTATCCTAAAAGTGGCACCAGAAGTTGTACTTGACGCTCCT
AGCTGCCGTCTTCTCTCTCTGCCCTGAGGTCCAGGTCAGCTTGGCAGTGTCTCATCCCTTATGCTGGGCATCTCGTCAACTCAGTCACATTGGA
ACATATGAGAGTAGGCCAGGTCAGCCCTGCCCTCCTCTGCTCTCTGCACCTCTACAGGCATGCCTGCTATTGCATAAATGCCAGAGAGGCTCAG
ACATGTGCTGTGTGGGGGTCCAGATGGAATAGGGAGGAGCCTTTGATATGTGCCTTCTGAGGGTCCAGGACTTTGTTGAGGACAGCTGGAGCAA
GTGGGCATGAACCTTGCAGAAAGACTGCTGACGGACAGCACAGCCCTGGGCTAGGCCTTCGCATGCGTCACCACAGTCAGGTCCAGGCTTGCTG
AAAGAGTGAACCTGATGTATCCTGTGACACTGTTACTTGCAGAGCTCCAGAAGCTATGGCTTAGAGAGCTGGCCTGTCCCATGTGTCCTCCCAT
TTCTGAGCTTTTGTTTGGTTGGGCAAAGGGTTTTCCCGCTGCCCCCTAGAAAATAGTGCCTCATCTCAACCATCTTGGCCACTGTTTAGCACAG
CAGGGCTGCTGGTGTAACTTGGCTGGGGATGGGTCCGGAGCATCACGATTCTGCAAGTTCTTGATGGTGATTCCAATGTGGCCGCTCAGGCTGA
CAGCCGTTGTGCTGGTGGTCATGCCCCAGTGACAAACTGAGACCCAGAGCAGGGCAAAGTCTTGTTCACATTCATGTCACCTGTGGCAAAATGA
GGCCTGGGGTCCTGTGTCTCTAGCTAGACCTGCAAGCCACAGTTCTTTCTGATAATATACAAGGGACCAAGCAGAGCATGGCTCCTTATGACTG
TGGGTGGGTTCCGTGGGTGGGAGACTGCTGCTTCCTCCACCTTTGTTAATATGACTCTGTCCTGCTACCCCTAGCCTGTCCTGACCCCAGATGA
CTCTGGGTGGTCTTGGCTGACGTGTCCTGAGTTAAGAAGGTTCCAGGAGCTGGTGATTGTGTTTGTGTGTGGCTTTGCTTTTGGGAGCTCTTGC
TAATTTACAGACTCTTAGTTGTGTTGTCACCCCAGGGCTTTGGGGGTGATGTTGTAGATCGACATCCTTATGAAATTACTGGTGGACCCAGTAA
AGAGTAGGTACAAAGGTACTCATGTCTTGAGTGTTAACCTGTGCAAGCTCTGTGTTAGGTGCTCTGCATGCAGTAAGTCTCAGCCTGGGGCATT
TGGGACGCATGGGCAGTGCTTTCAGACGTTCTAGGTAAGCACAGCTTCATGCGTGGGTACTCCTGGTTTTGCAGGAGTAAAAGGTAAGTGTATT
ATTGAACATTCTACACTGTACAGTAATCTCCTCTCCTGACCCAGACTCTGATTTCCCATGGCGGCGTAATTAGGGCCCTATAGGCTTCACTCTA
GTCTTATAGAGGCCCTGTGGTCCCCAGGGATAGTCTCAAGTCTGTCTGAGCTCAAGGCCTTTGTCCTGAGCTGGGTATCGAAGCCGCGAGCTTT
CCTTTGACTGATTACACGCGCTTGGCTGAAAGGACTCAGAAGTAAGGGTGGAGGAACCGAGAGCTGCTGGGGCTTCTGTGATTCTGGAAGCATC
CAGAGGTGTGGGTTGGGATCCGAGTTAGCCTCAACACCCTGCTTCCTCCTGGGTGGAACTCTTGTTTCAAAACTATAGCAAAAGGCAGGTAGAA
CCGGAATGCTCTTTGAATTCTAAAGCCAGAGCAAGGTCAGGTTGAAGAACCTCAGCAGAATCAGTTATCCTGGTGCCTTTGCTAGGGTAGCATG
TCTCTTGGTGGATGCTCACGGGTGCCTCGCATCTTAACTAGAATACCACAGCAGCCACGCCATGAGGAAGGAGCCTGTGGTGGTGCCCCTGAGG
GAGAAGAGATTGTTCTTCTCTGAACATACATGTTCAGAAGTAGGGACATGAGAGACACTGGAAGATGGGAGACAAGTCACAGGTGACAGAATAA
GCTTTCTGTGGTTGCAACATGATGGACACCAGCAACCAGTGCCACAGCCACTGCAGGCCACTGTAGGCTGTGTTTTAAACTCACTTGATGTGTG
TGTGTGTGTGTGTGTGTGTGTGTGTGTGTGTGTGTGAGAAGATGCCCAAAGAGGCTAGAGAGGGGCAACAGATCCATTGGAGTTTGAATGAAGG
GCATTTGTGAATTACCTGATGTGATTTCTGAGATCCAAATTCTAATACTCAGCAGAGAGTTGTAGTAAATGTCCTTAACCCCTGAGCTGCTTCT
CCAGTCCCTTAAGTGGTTAAAGCTCGTGTACGTAGATGGAACCCTCAGAAACTGGTCTGGACGGGGAGAACGAGGCAGCAAAAGCTGAGTGAAC
TTGAACTTCCTGGTGCTCAAGGCTGATGAGAGGTAGGGCAGCCTACCCTGCAGTCACTGGGCTCCTACTACCTCCTTCCTGCCTTCCCTCTGAG
GAGGCATCGCTGTAGGAGGAGGGTGTGGTGGGGCCTTGGTGGCCCCAGATGTCCCCTGGCTTCCCAGATGGGGCCAGTGATTAGGAAGGAGGAC
CATCCTCATTAACGGTCATTTACCCAATTATGCTTTGTGGGATATTCAACCCAAGGAACGTGCTGCCCCTGGCCGAGCGTAAGAGGAAGCCTAA
ATTCTCCAAGGAGGAGCTGGACATTCTGGTCACAGAGGTGACCCGTCACGAAGCCCTTCTCTTTGGGAGAGAGACCATGCGGCTGTCCCATGCC
GACAGGGACAAGATTTGGGAAGGCATAGCTCGGAAAATCACCTCCGTCAGCCAGGTTCCCCGCTCCATCAAGGACATTAAGCACAGGTGGGATG
ACATGAAGCGCAGGACCAAGGACAAGCTGGCTTTCATGCGGCAGTCACTGTCGGGCCCAGGGATCGGGGGCCACACCTACCCCATCATGCTCGC
TGCCCACGAGAGAGCCATCGAGGCAGCACTGCTCACCACCAGGGCCGGGCATGGCTTCCCCAGGGCAGAGCTGGACAGAACCGACAGCCCGTCT
ACCAGCTGTGAGTATCGATGCCGAGTGTGGGGCTTGCTTCCTCGTGTGCACTTATTCCCATGCTAGCTCTCAGCCCTGCCTGTCTCCCTGACTC
TCTCCATGGCCCCATCTCGCTTCTGTCTTGCCTCCACTTCTTGCTCATCCACTGTGTGCCAGAAGCCCGCTTGGGAGCAGGAAGGACAGGGCTG
CATCTAAAACCTACACAGACACTTGTCGGACTAAATAAGCCCCAGGTGGATCCATCCTGTGGCCTAAGGGTTACGTGCACATAGCTATGAAGGT
GGCCGAACCCAAACCATAACCTTGTTTAAAACAGTACAAGTGTTTTGTTGGTTTGTTTGGTACCTGGAGCAGAAACTGTGGTTGACCTTGTGTT
ACAGTGTCAAAAGGTTAGACGTGCCTTCTTGGATTAGATTATTGTTCACCTTAGAGGAAGGATAAGGGCTCACCCAGTCCAGGACATCCCAGAT
TTCCAGCTTTGAGCTTGTGTGATCTCCAGGCCTCCCACCCTGGGCCTGCTGCTCTGCAGTCTGTGCTGGGTCTGGGACACACATGAATGGAGGG
CTGCAGCTGTAGAGAAGGCCTGAGGCTGGCTGAGGAAGATGTTTGAAGGGCGAGGAAGAGTTAACCAGGCCTGGTGCGAGGCGCAGAGGAGGGG
AGGGGAAGGCGAGCAGGCTGGGAACAGCATGTACAAGGCCCACATCAGGAAGGCGCTTGGCACGTTCCAGGAACTGCAGAAGCCAGGGAAGCCC
CATCCTAGCCAGCCGGCAGGGGGAGGGGCACCACCACAGCAGCCGAGTGGCAGGGCTGGGCTGGGCCGGGGAGAGACCTTTCTCCGTGACTCTG
TCTGCTTTGTCCACTTCTCTCCCGTCTTCTGAGCTTGATCCACTTCTCTTTGGCTCCTGCTTCTTGGTGCTCACCTGGGACTGGCTGGCCCTGC
TTTCTTGCTTCCTGGGACTTTTAGCGGTTCTAGCTGTTCTAGCTGTGTTCACCAGGAGGGGGAGCCATCGGAGTCCATCCCTGAAGAACTATCC
CCTCCCTCCCACTTCTTCACACACCCATACCCTTCCTCATCTGCTGTCCTCAGTGGAGTCCTGTGAGCAGGGCAGGAGGATCCAAGGAGGCTGT
GGGGCCGAGCATTCAGCCAGCGGGAGAGTGGTTCACCAAGTCCGACTTCCTCCCTGGCCCTTTGCCGCGGGGATCTGCTTATTTCCTGTTTGGC
TGAGGTCTCTGACAGGTGGCCACTCACAAAGTCAGCCTCGGGAGTAGAAAAGAGTGGTCATGATGAGGGTGACTTCCTTGTTAACCAGATGAGG
CAAACAGTCTTCCAGAAAGTGGAGGTGGCCCTGAGACATCAGAACCAGACTTGGGCCTGGACCTGCCAGGGTTTGCTAAAACCTCGGGGCGGGC
AGCATCTGGAAAAGCCTATTTCCTGAATCCTGCCCTGTTCACCCCTCCGTCTCCTAGAGATCGAAGATGATGAGATTAGCAGAGGTAACATGGA
CTGCAGGGGCTCCCAAATCTACTAACTAATCACAAGCTTAGCCTGGCTGGGACCGTAGTGGCCTCAGGGCTTGGGGAAAGGGTAATGGCCGTTC
TGAGTGCTCCTTTCTACCCTTTCTGGCATCCCAAGAGGTTGGCTGCCACTTATGGAGTATGCTCAGGAAGCAGTAGGCTCCACTGCGCCAGCAT
CAGGAGACCCACAGATCAGTGTGGTCAGATCAAGCACTCACGGGTGGGCCATGATGTGCCTGACCTCACAGCCCACATCAGGGGGACAACAAGG
CTGGTGGCTCGCCCCACATAATCCTGAGTCCTGCATCAGCCCTTCCCTCTCATCTCCAGCCATTTGGCATCTCCCTGTGGTGGTTTGTTAGGAT
TCCAGCAAGGCCACAAGGTGGCCCCAAGCAACTTTGAAAAGTCCCTCCCTTGTGGAGACCAGTGCTCACAAGAGAGCAGCTTAGAACCTCTCAC
AGGGATGGGTAGCTCTCTCAGCTCTGCAATACACCGTAGGGTCTGGGAGCCCCACTAAGAGCCCAGCCGGGGCCCCGGGGGTTCAGCAGAGCAG
TCATTATTGTTTCTCTGAAGGTAGTGAAATGTAATGGCACAATCGTGGTCATGAGCCCTTAGGCTGGCATGGCTGCCACTGGAGCTGGGGTCTC
CTTTGCTGGACAATCCTTTCCTGGAATGTAGCTGCCTCTGTGGGAAGAAGGGAACCAGCTCCCGCTCACCCTTCTACCGATGCTGGACCTACAC
GTCCCAGGGACTTAGAAGCTGGCATTTCTCTGTGTGTGGTGCCAGTGTAGGCTGACGGGTGGCCAGCAGGGACTTTTCAAAGCTGTGACCTGTG
ACCTGGGCTGCCCCCTCCACCCTTTTGTAATCACCTGTCCTTCTCTCCTTGTCTGATCCAAGATGATGAAGACGAGGAAGCTCCCGGGCCCTCA
CAGCAGCCTCTCCGGATGCCCCTGCTTCGTCCTCCAGAGGAAGAGGCCCCCCTGGCCAGGCCCACGCTGCTCTATTCATCTTCCTCAGACCCAT
CCAAGATGTTGGACCCTAAGCCAGAGGTCCTGCCCCATCCCTCACTCCAGGCCCGCAGGACCCCAGAACCACATCCCAGCTCGCCTACCCTGGG
CCTGGACTGGCATCTCCTGCATGCCCATGCCCATCAGACTGAAATGTTCCAGAAGTTCTGCCAGGAGCTGATGACTGTGCACCGAGACATGGCC
GGCAGCATGCATGTCATCAGCCAGGCCATGGCTGAGCTGAACAGCCGTGTTGGTCAGATGTGTGAGACGCTTATGGAGATCCGAGATGCAGTTC
AGGCATCTCAGCGTGGGCCAGAAGGGGCAGCCCCCATGGACCACACTTTCCAGTCTCAGGCACCCCTGCTGGGCCCTACACCAGCACCCCCAGA
ACCAGCCCCAATACGGACTACCAGGTCTCGGAAGAGAAAGCACAATTTCTAACCTGGCCAGTTTGCCTAAGGAGAAGCAATGCCGGTGGTGTCT
GGCCTTGAGTTGGGGGAGTCTCTTGTGTCCAGAAACAAGACTGATGACCGTGGGGCACAGTCGTCTCTTCACCTGGTGCAGTCCAGTCTCCTGT
GCTTTCACCGAGCGGGTGTACAGGCGCACCACCCCCTTATCTCAGGAAGATACTCAAGACACAAGAAAAGCTGCCTGCTTGAATGCTTGCTTGG
CCTAGAAGGCTGCTCTCCTCGTGGCCTGTATCCTGTATCCTATGGAGCAGGCCTTGCTGGGGCAGGGGGCAGCATCCAACAGAATCTTGCTTTC
AACTCAGGGCTGCTGCAGAGCTGACCTCCCAGGTCCTGATTCTGCCCTTTCTCAGGTGGTGCGGGGCTGTGGGTGGGCGGGGCTGGCTCAGCTG
GGTGGGTGGGGCCCGGCCATGGCCCTGTGCTTGCCCTGCGTTGCTCGGCTGCTGTGCTTGCCTTTGGGAGGGGTTGGTGGGGGGAGAAGAATGG
GATGTGCTGGTGAGGGGAAAGAGATGATTTTGCATTCTTAGCATACTATAAACTTTTCCCCCCCCCCCCCCGAGCCTGCACATGGCAGGCCATC
TCAGGAAGCCAAGTCAGTCTTTACAATCACTGATTACTGGTCATTCCAGTCATTGTCATGCATCTCAGGGTTCAGAAATGATACAGCCATGATA
GTGGGGACCTGGCCTGCTTTCTTCAAGAGGCCAGATCTGGAGTACTAATAATAGATGAGGATCCTGCTCCTCACCTAAGCTCTGCCTCAGCTTC
TACAGTTAGAAGAGGGAACAAAACCAGCGTGATGAGTATGCCAGCCTGTGATGGCCTTGAGTCTCACAGCCAATGAAGGGGAGGTTCTGGGCCC
TGGCCTCAGGAGCCAGCAGGCAGACTGTCCGCTCACTGGCTCCTAATAAGGCCATAAACCAATGAGACTGTGTCCAGTCCAGCTCCCAGCATGC
CTTGCCTCTGCTTGCTTGGGCTGTATACTCACCTAGCCATACAGACTTGTAGCTTTGAGTTTTGTTTAGGGGGCCCAGTCAGAGAGCTGGGATC
TTTGAGTGAGGGAGGAGAAAAGAAGCCAAAGGCTTCACTCTGACCTAGGTGCAGCTTTGACCTCGATCCCTGGCCTGAGGCTGACCCCAGCAGA
CCTTTCACACCCTACCCTTTCTCCCTAAGAAACAGAGTGGACCTGGAAGCCTACTGGACCCAGTGCTCACTGAGGCTGTTGCTAGCGCTGTTTA
TAAACACACACCATGCTTCGTCCTACATGAGATGATGGACGGATGGCACCTGCTGTGCCCCTGAGCCCTGGGCTCCATTGCCTCAATCTGTGGT
TCTTTGGTGGTTCCTGCCCCATCCATCAGACTGCATATTGTCTTGTCTAGTAATAGCCTGGTGCCTGGAATTGCCCCGCCTGTCCATGCTGGAA
CCCAAGTTGGCCTTCTCAAATATTTTTTGTTAAGATGGCAATAAAATGAATCAGATGTCACACTTTGAACACAGCCAGGCTCCAGGATCTTTTT
TTGGTGTGTATTCTCTTTCTTCCTATCCCAGGGAACCCCCATGAGCCTCCAGATCAGGGATTAGATGCCTAATGGAAAGTGTATGGGGAAGAGG
TCCCTGTGGACCTTGCATATTCTGGATTCCATAAACTCCCTAGGGGCGTGCTCAACCCCATGCTTGGCCCTCCAAACGAAAGGCAGTGACTTGT
AGGCCAAAGGCAGCTGAACATCCATGGATGTCAACTTTTAATTTTTCCTGCACATCAAAAGTAAATTTAACCTTATGCTCCCCCAAAGTATTAC
AGTTTCCAGGGAAGGAAATAGTTTCTGGCTGGAGACTTGAGAGGCTCCTGGCTGTCCATTAGGGGTTATCCTGGGATACTCCTCAGTAGTGGCT
ACATCTCCACTGACCTCAGTAGCTGCCTGTGAAAGGCAAGGCCCTGCCTACCAAGACTCCTCAGAAGAACCAGAGTTCTTCCTCTTGGATTCAC
TGGAGTGTGCCTCACCACAGAGCTGTGTTCTGACCCGTCAGCCTGGCTAACCTCCTGTCATGGAGAGAGCTTCCGTGAGAGGAATCCTCAAATG
GTAGCCTATGATGTACTTTCTGAATAGTTCTTAGCTACCTTTGGCTTTGGAATCCAGATCATTCTCTTAAGTTCCTGACCAAAATGCATAACCA
TGGAAATTAAGATTGCAGTTGGCAGGAGATACAGAGATGCAGCTGGCCTGGGTCTCCTCTAATACCCAAGACCTGTGAGACTGGTTGGCCTCTA
TTTCTGGTGTTGGGGAAGAAGAGGCCACATTAAAGGCTGTGTACAAGAACCAAGGTCAGAGTCCAGGGACAGTGGTCAGTTTGAGATGGAAATG
GTTTCCATTTTGGTGTCTTGGTGGCAGTGGGGACCAGGAAGCCAGAATATATTCAATATATTCAATAGGAAGAGGAAATGAATGGGGAGCCTGT
CCCAGCTCTTCCCAAAAGGAAGCCTTGAAAGGAAGCCCATAGCATATGTGGGCAACTGCCTGGATATCAGAGACCTAACCCGGTGACATCACAG
AGCCCAGCTTCTTGTCCAGTCTTCCCATTTCTGTCTTCCTGGGCAACTTCCTTTCTGGTAGAAAGCTGCGTTGGCCATGACAGGCTTGTGTCAG
TCCCATTAAACAAAGTGGACGACTCCCTGAGCCATTTTCTTTCTTGAGTCGGAACCCATAGTCATTTCCTGACCCAGCTAGAAAATATGGTGCA
CTTGATAAGACCCAGTGGGTGAGGCCTGAGAATTGTCTCCCGGTGCTTATCTGTCCAGTACCCAAGGCTAGTGAGGAGACAGGACTCAAAACAA
TGTGTTATCTTCTGGGAAGATTCAGTCCAGAACCCCTGCCCTTCCCACCACTGTCAGTCACCTACTTATTTCTAGGATGCCTGACTCTGAACTG
CCAGACTTGAACTCAGTCTCTACAGGACAGCCTGGACCCGGGTGTTTCCCACCCTGCTCTGTTTTCCCAACAAGGGAGTGTGAGCTAGGAATTC
AGACCCCCTGGTAGACAACTGGTCTTGACCACAAGGAGGAGACAGTTCCTGGAAACCCTGGGGTCCTTGGCCACGTGACACAGGACAGTATTTC
GTAAGAATGAGCACAGCAGAATTCCGAGAATGTGCCAAGCCACTGAGTGTCGCCACTGTAAAGGGAGGGGACCTCATTAGAGGTGATGGGTCCT
GAGTCTTAGCTGGATGCAGTAAACTTTCTCTTGAAAGTTCTGTCACATAAACATGTTAAGTCCTGGGACTCGGTGACTGTGATTCACCCACAGT
TGCCATCTGTATCTCATTTCAAAAGCCAAGTTTTAAAACAACCAAATGCAAGATGCTCAACACATTTTGATCTGATGCTGGAAATACTTTGTGA
TCTGTATCAAGACTGAGTGAGAGTGCTCTCTCTAAATGCTGGCTCTTGTTAGGTAGAACCACATTGCAGGGGGAGGGGGGACGAAGGGAAATGC
CTGCAAACCATTTGAAATGGTTAGACCCACTCCAGACATTGCTGTTTTGAATCAGCAAATCGTGATAGTGACTTGTTCCCCATGTACCCTTTTC
TCTGGCATTTAATATTGAATGGCTGGCCAGAAACTGAAAGGGAGAAGGGAGAAGGTGCTCTTGGGTGTTCTGTGAACCAGTGGTTCCCCAGAGA
ACACAAGAATGGGATTTCTACCTTCCAGATGTGTCTGGAACAGAAAAACTTGGAGCTACATTCATTCTGGGTCCTCCTGACCATTACACTGGAT
GGTTTGGGACCAATCCTAGACTCTTGTTAAATTGGAAACTTGGAGCTGAGGCTGGTCCTGTGAGTGGAGCAATAAGGCTGAGCATGGTTGCTGC
AGTCTTGGGGTCTGCCTTTTGTCCTTGCTGACTATGTTGTGGCTGGGTGCTTGGAGAGCCTCATGAGATTTTCACAGCTCATGGGGTTAGGGGG
CTCTCTGATGCTCATGTAAGATACAGGTGGGGATGCAAGCGTGCTGAGGCTAAGGCCCTGGGCAAGACGGCTGACCCAGGGCTGTGAGCACCAT
TGATCTTGTCCAGATTGGAGGAGCTTGGTGTTTGCTCTCCCACTGCTTGGGTCTTCATCCCTCAGCTTACAATGTCCTCTGTCGCCAGAATGGA
TTGTTAACTGGGCTCCAGGGGATGTGTTTCTAAGGTGCCAGCCTCCTCTGGGGTAGTCAAGTTGATTCTTCAGGACCTGTCCAGATAAGAGCAG
ATGGGATAGGAGGTCTGCAGGCACAGGTCTAGCGGTCTCCAGTGTAAGTTCTTGGAGCTGTCAGCCCCCCATTGTCACCTTGCAAAGAATCTGA
CATCTGTGTGAATGTTTTCTCTTTGCCTCTTGAGTGAGAGAAGCAGCACAAACATTATTGAGGAAACCTGGAAGGTACAGACAATTCCAGAGAC
GATCCACCCAAAGTGATGGCCGTCCTGGTTAAAACACGGGTGTGTTGACTTCAAGCAGTTCTGCTGGGAGACACATTGTTAGAGCACAGTATAC
TTGGCTTTGGCTCCAACCTGTCTCACTCTGCATCACAGCAACACTGTTTTGGAGTTGGGTAAGGTGATTCCAGGATGTTGGAAGTGTCATTTGA
AGGAAACTGGGTGAGGGGTGTGGATTCATTGGTGTCTTGGGCATTGCTGAGTCCATGCGTTCCGGGCCCACATGGAAACTGTGAGTTGTAGCTC
AGGGAATGATAGGCCACAAATTTGCATCATTAGCTGTGCCTTTGGACAAGTTTCTCAGCCTCTCCAAGCCTTTCTGTTGTTGCCGATCTTCTGC
TGTCTCTCCTTTATCTATTCCCCCTTTGTCCTTATTGGGCCAACTATAAGGATGCTGCTCTAAAACTGAGCTATCATCAGTCACTCTTAGAGGA
TCGGTTAATTCTGCTAAGCCTAGCAAAATATGGAAACAGCCCAAAGCTTCCCCCTGCCCTAACTGAGGTTAAAGGTCAGTGAAAGTGGAGCTGG
AGCTTAAGAGCCTGCTCTTTCATAGGACCCAGGTTTGAGTCCCAGCACTCACATGGTAGTTCACAACCATCTGTAACTCCACTTTCAGGAGATC
TAATGGCCTCTTCTGGCCTCTGGGAGGGCTCTATCATATGGTACACAGACATAAATGCGGGAAAAACACCCCTACACACAAAATATTAAAATAA
AATAAGTTTTTTTTAAGGTCAATAAAGAGGAGAAGGGATAGCCACCTGCTATTGAGTCTGGGGTTGACTGTGACTCTCAAGGTTGGCAGGGCAG
ATATAATTTCCAGTCTCAGATTAAACAGGTTCCTAGGGTCCAGAGAATGTGTTAACATGATACTGAATAGAGATGTAAGTTAGGAGGACCAGCT
CTATAGTTGGCTGTCTCTGGATGAGACAGCAGCTCTTCTTATGTGCCCACATACTTTAAGCTCTTGTGCCCTGCACCTAAGGCACTCAACCAGG
TGTGGCCCATGGTTCTCACCTGTCCCCTCCCCACCAGGTACGTGGTCATTTCCCGGGAGGAGAGGGAGCAGAACCTCCTGGCGTTCCAGCACAG
CGAGCGCATCTACTTCCGGGCATGCCGAGACATCCGGCCAGGAGAGCGGCTGCGGGTCTGGTACAGCGAGGACTACATGAAGCGCCTGCATAGC
ATGTCCCAGGAAACCATCCACCGCAACTTAGCCCGGGGTGAGTATGGCCATGGGAAGGAGCGAGTGTGCCCTCAGGGCCAGGGACATGCCCATA
ATGCTAAGTCCACAAGTCTAGGTCAGTACTGATTGTGAAGTGACTCGCCCACAAAGCTAGGGTCACGCCTACAGGACCAAGACCACGCCCACGG
GGCTAAGGCCATGTCAACCAGCAAAGGTTACACCCACTCTGAACCACAAAAGCCCAGAAAGCACCTCTGGGAACCTTCCAAGCAAACCAATGAG
GAATGCAGTATCGAGCAGTGCAAAGAGCCAGGACAGGCAAGACCTCAGCAAAGTAAGCCAGTGGCCACCCCGAGGGAATTCTCTAGTGTGCAGT
CCTGAGAGATCAGCAAATCCCAACCAAATAAATCAGTTGAATAACACCAATCTTGTTACCCCAACACTAACTGATGAAAAGACACTTCTCACTC
AGTACCTCAATAGATAACTTCCTGTGTGCTAGCTGGGTTGCCTTCACTTTGCCATGGGATCAAGGTAATGAGAACTAACCTGCCAAGATGTTCT
GAGAGTCCGTGGAGCAATGTACTTACCACCGTTGATGCAATCTCTACCTCGACTGTCACTAAGTCCTTGGCTGTAAAATTCTAGACCAGTGATT
TTCGTGACATCAGCTCCACCATTGTACAATTGTCCAAACTGAGCTTTACAGAAATGCAGTGACTTACCCAGGGTCACGTGACTAATGAGGACAG
AGCCAGCTTTCCAACTTGAGGCATCTGAAGTGAGGATCTAAGTTTTATCCAGACAAAGCCAGCCTTGGTGTTGTTGATCTTGTTCCTGGGAGCC
AAAATGATACACAGCATGAAGGAATGGTTTCTACCGGAGGAAGGCTAGTACAGGAGAAGATGGGCTCAGGCTGGGAGAACATAGTGGGTTGGCT
GTCCATCCATGTGCCACTGGAATGGCTCATTTAAAAGGAACTTGGCTTACTCCCTGCCTCTGGCTCTCCAGCTGTAGATGCCATCCTCAGCCTG
GCCAGGGTACACAGGTGCACCCAGGTCCTTTTTTCCAAGAGAGCCATCTTTGCACAGAAGCTGTACATTCACACACCCAGAGTCAATTTGGTCC
TCGGAGGCAGAGCCATGCCCAACGTCTTCTCCGCCGTCATCTTAACTAACCTCTTCACTGGATAGCCTTGTTTAGAAGATGAGCGGAGGAGATG
GGAGCAGATGACTCACTGAGGTGAAGATGATTAGGAAGACAAAGCAGAACAGGTCTTGGGGTGCCCAAAAGCTGTCTGACTTAGTCAAAAGCAT
CTGGCAGTTGCCAGCGAGCTGAGAGACCTGTGGGTAAGCCTATCTCCTGACTCCTTTGGAGCTGAGCTTGGGCCATGCTGGTTTCATTGGGATG
GCATGCTTCTGGGATAAAGGGTGGCTACTGTTTGGCCTCTTCCTGAAATTCCAGAAGGGTTCCTGTGGGCCTCGGAACCTCCAGAAATGTTTGG
TGAACCTCAGTTGCATCGATAAAAGCAGAAGTGAACGAAAACAGCCAGGGGGTTTCCAGAAGAATCACATTACCCAAAGAAAAGAAAACCAAGA
TGGGTCACAGCTGCTGTGGCACAGATTCATCTCTTTCCCACCATCTTGGATTCTTCTGGAATATGTCTTCCTGTCTTCTCCAAAGCACCTCAAC
CTGAATCATCCTCAAGAAGACCTGAGACCCAGGGACATCTCAAAGAGAGGAGAGAGCTTGGGGAAATGAAGGCTCACAGGAAGATTGAGAAGAG
AGAGAGAGCGAGCCTTGGAAGATACAGGATTAAGGTATCTATCTGCCCGTGACCAGGCTGGATTGTAGGCTTCAGGAAGAAACCAGCAACCACA
GTGGACCCAGCAGGAGCTGTGATGGAAGTGGTCCTTGAGAGTGACCGTGGGACTTGGCAGCTGATGGATGCATGCTAGATGTTAGGTACAGAGT
TCTAGACCTTTAAATGTGTCTTGGATGACCTATCAGCCCTGCTTAGAAAGGTCCCCATTGCCCAGTCACTTAAGGTATTACAGCTATATTAGCT
TTGTGTGTGTATGTGTGTGCGTGTGTGTACATGTGTGTGTATATGTATATGTGCTATACATGTATATGTGTGTGCGTGTGTGTACATGTGTGTG
TACATGTGTTTGTGTATGTGCATATGTGTGTTCATATGCAGGTGCATGCATGTATGAATCAGCCTCATGTGTCATTCCTCAGTAATCGCTTTGC
TTTTGAGACAACTCTCACTGACCTGGAACTTGAAAAATGTGCTAGGCTGGCTGGTCAGTGCACCTCAGGGGTCTACCTGAACCCACCGTCCCAG
CACTGGGATTGCAAGTGCCTGCCACAACACAGAGCTTTTTTTTTTTTTTTTTTTTTTTTTTTTTGGTTGTTTTTAAATGTGAATTCTGGAGATT
GAAACTTAAGTCCTCATACTTGCAAAGCAAACGTCTTATCAGCTGAGCTATCTGCCAGTCCCCCCGCGCCCCCTTTGTGAGACACAGTTTCATC
TATCCCGGGTTGGCCTTGAATTCCATATGAAGCTGAGGCTGGCTTTGAACTCCAGAGCCTCCGTCTCTACCTCCCAAGCACTGGGGCCATTGAT
GGGCACCACTGCATCTTGCTTTTAGAGAGAGGAGCATCTTCATGGTCAGAGGCTGCTTAGCCAGATCGTACTTATTGTCGTTTTTTTTTTTTTT
TTTTTTTTTAATGAAAGAGTGTTGTGTGGAGCCCAGAGCTCCTGGAGAGGATATTCTTTATTCCTTTGTGGTTATCTTTGAAGGATACGAGTCG
ACACACTTAGGTGATCTGTGTGATTTGTGTGGGGATTATAAATTTTTTTCTTTGATTTTTATCCATGAATGTCGTGGAGGATTTGGAATAGAAG
TCATGGTGACCTTTATTAAAGGTTGTCCGTGGACCAGGGATTTTACATGGAAGCCACAAGTTCAGAAGGCTTTCCTGCTCAGCCTAGAAAACAT
GGGTGACTTTATCTTCCATTACAGAGACAATGAGAGTCTTCTGTGATGTAACACCCAGCATCTTCAACGGTCTGATGTCTGGGGAGCCCAAGAC
GAGGCAATAGGGCACCCTTGACTTTTCAGAGATTCAGCTTGGCGAGCACCCCACCTGTCCTGTGACACAAATACCTTGCATTACATTTTTAAAA
TTGAATTTGATTTATGTAGTGTATGTAATGGGTGTTTTGTCTTCATATATGCCTGTGTACCGCATGTATGCCAGCTGCCCTTTGAGGTATCTTC
TGGGTACCCTGGGACTAGAGTCTGTCACAGAGGGTTGTGAGCTGTCATGTGGGTGCTGGGAACTGAACTCAGGTCTTTTGGAAGAGCATCCAGA
GCTTCCAATCTCTGAGATGTCTCTCCAGCTCACAAAAGGCATTTTTTAAAGGGCTCCATCTCGCCCCCGAGCTTTCTGCAGTTCTGCTGGCACA
GATACAGGGGCTTCTGGGAGCCGCCCGAGTGCCAGGAGTCCGGGATGAGGTTGTTGCTTAAGCAGCGCCTGTGAGGACAGAATAGGGACTTGAG
TGGCTTAAACCCTGCTGTGCTGTTTTCTGTGGGAGCCTAGAGGCGTGAGGGAAGCAGCTGGGGGAGGCCAAGCATGGAGCCTCCTGACCTTCAC
CCAGGGCCTATCAAATCCTTTCACAGAAGCCTGGACACTGAACAGAGGCTCAAGGTTGCAGCACCAGGACCCTGCCCAATTCAGGTGGCCTTTG
AGATTGCGCAACCCCACAGGTTCTGCTGGCAGAACCTTCTGAAGGCTGGGAAACAACTCTCCTCAGCATGTCACCCCCTGGGCATCCCAGCACT
TCTGTAGCAGTCTGTGCTGGCCTCGCTAAAGGATCTAAAATAGCTTACTATTGGTGATCTCACCAACATTTCAAGGTAATTAAAAATGAAGGAA
AGAGAGGCATCTTTTGACAGGTGGGGAAATGGCTGTATCTGAGAATACTTTGGGTTTCGCAACTACCTTGCCCTAATCTTGAGGGAGGCTGATA
CTCTGGTATGTTTAGGAATCTTTCCCAAGGCACCCCCACTTCTTTTTTCAACCTTAGCCATGACTGAGAACCACAAGACTTTGACCCATCTTTC
TTTGTGTATTTCTTATCTTTAGACTGCTGGGAGAATAGAGAGGCAGCAGCTGTAGGGTGGCTGTTTCCCTTGGCTCTCTGGGCCCATGAGCATG
GGAGGAAGGCAGGGCCTGTGCTTCCCCCGTGCTTTCTACTGAGGCCCTGCAACCACCGTGTAGTCTCTAGGTCTGAAGGATAGCTTGTCCCATG
GCGGGCAGACAGACACTCAGCTCTGAAAGCTGTTGCATTAGTCAGATGGAGTGAGATGCTGTCTCCTGGGGTCATAGTATGGCAAGCCCCATCC
CCATAGGATGATGACTCCTTTTCAGTCTCCGCTTGAGACATATGCCAACTCACAAGTGTAGCATTTATCCCATGTGAGGCCTTTCTGTAAAGGT
CCTGTGACTCTAGAGCCCACGATCCGTTCTTCTCTCCATTTGGGTCTCACCTGACCCAGATGGTCAGATCCAGGTGGATGTGTCAGGCAATCCT
CTCGCAGAAGGCCTGCTCTGTTCCCTGTCAATTTCTCTGTTTGGGGCTTTCCCCCACATCTTCACACCCTGGTTTTCCTTCCTAGGAGAGAAGA
GGTTGCAGAGAGAGAAGGCTGAGCAAGCCCTGGAAAACCCAGAAGACCTAAGGGGTCCCACTCAGTTCCCTGTGTTGAAGCAGGGCAGAAGTCC
TTACAAGCGCAGCTTTGATGAGGGAGACATACACCCTCAGGCAAAGAAGAAGAAAATAGATCTCATCTTCAAGGATGTGCTAGAGGCCTCACTG
GAGTCTGGTAACGTGGAAGCCCGCCAGCTGGCCCTGAGCACCTCCCTGGTCATCCGGAAAGTCCCCAAGTACCAGGACGATGACTATGGTAGAG
CTGCCCTGACGCAAGGCATCTGCAGGACCCCGGGGGAAGGGGACTGGAAGGTCCCTCAAAGGGTGGCCAAAGAGCTGGGCCCACTGGAAGATGA
AGAGGAGGAGCCTACATCATTCAAGGCTGACAGCCCTGCAGAGGCCTCTCTGGCATCTGACCCCCATGAGCTGCCTACCACCTCCTTTTGTCCT
AACTGCATTCGCCTGAAAAAGAAAGTGCGGGAATTGCAGGCGGAGCTAGACATGCTTAAGTCTGGAAAGCTGCCTGAGCCCTCCCTTCTGCCAC
CACAGGTGCTTGAGCTCCCAGAGTTCTCAGATCCTGCAGGTAAGTTCCTCAGGATGAGGTTGCTGTTAAAGGGAAGAGTGTGCAGTGCCACTCG
TGCGCACTGTGTGGAGGGTGGCCCAGAGCGCTCTGCTCTGTCACAACCACCTGCCAGGAGACCAGAGGAATGCAACTCAGCAGAGGCAGCCCCC
TCTTGGGGTGGGAGCCCAGTCCCTCAGGTGACAGAGGCAGGCTGGCTTGAAGATCGGGGGAGGGAGGGCATTGCATGTGCCAACCTGTATATCC
TCCTTGTCAATAAAATATGTCCCATTTGCAGGAGGGCTGTGTGCTGTGTTCTTGACCTGGTGTTTTCTCTTGGGGGCGGGGCCCACTGTTGTCT
CCCCTGACCCAACTTCTCTCTTCCCCCAGTCTCTGCTGGGAGCTTTTACAGCTCCCCAAACTCAGGCCAGGAGCACAGCCTCGTCCACTTCTTC
CAGACTCTGTCCCACCCACATGGTGCTCTGTTCCCAGCCTGGAGTTCTAGAAGGATGAGGAACCAGAGAACCTGGGGCCGACAGCTGTATGACT
GTCCTTTTTTGCTGGCTGCCTGGATCTCTTCAAGTTGTTGGACCAAGGAAGAACTGTGGTTCGATTCTGTGCAGATTTGCAGCTAAGCCGGGCT
GTTTCCCTCAGGTTTCCCTTCTCAGCCACCCTCAGAATGGGTTGGGGTGGACGTAAAAACCACATTTTGCCTCAATATCGAGGCTGCCCGCAGT
TAAGAGCCACATGCTCACCCTGACCATAGCGGTGGGCATTCCAGGGCCAGGTCCTCCAGTGTAACCTGAATGTGTGACATCAAACATTCTGCCT
CTTCTAAAACATTCCACCCCAGAAGCACCCACAAATCAAAACTGAGCTCAGGTGCAGGCAGCGTGGAGCATAGTGAGTAACGTCCAAGAACACC
ATGGGCAACTCATGGGGAGGGAACCTCACTGTGGAGAGTAGAGTTACCCTTGACTCAACTCCAGGCTGGGCTTCCATCTCTGTGGGGCCCGCGC
TCCCTCAGAGGCCCTTGCTGAGTCTGTGCTGTCAACAGCCCACGCGAACTTCTCCTTCCCCTCTTATTTTCCAGCCTCAGAGAGTATGGTCTCC
GGCCCCGCCATCATGGAGGACGATGACCAGGAAGTGGATTCTGCAGATGAATCTGTCTCCAACGACGTGATGACGGCAACCGATGAACCCTCCA
AGATGTCCTCTGCCACAGGACGCCGCATCCGGCGCTTCAAGCAGGAGTGGCTGAAAAAGTTCTGGTTCCTACGGTACTCCCCGACCCTGAATGA
GATGTGGTGTCACGTGTGCCGCCAGTACACGGTTCAGTCCTCACGCACTTCCGCTTTCATCATCGGCTCCAAGCAGTTTAAGATCCACACCATC
AAGCTGCACAGCCAGAGCAACCTGCACAAGAAATGCCTGCAGCTGTACAAACTGCGCATGCACCCGGAGAAGACTGAGGAGATGTGCCGCAACA
TGACCCTGCTCTTCAACACCGCCTACCACCTGGCGCTGGAGGGGAGGCCCTACCTCGACTTCCGGCCCCTGGCAGAGCTGCTGCGGAAGTGCGA
GCTCAAGGTGGTGGACCAGTATATGAATGAGGGGGACTGCCAGATCCTTATCCATCACATCGCCCGGGCACTGAGAGAAGACCTGGTGGAGCGC
ATCCGTCAGTCGCCCTGCCTCAGCATCATCCTGGATGGGCAGAGTGACGACCTTCTGGCTGACACAGTGGCCGTCTATGTGCAGTATACCAGCA
GTGATGGACCCCCAGCCACAGAATTCCTATCCCTGCAGGAGCTAGGCTTCTCCAGCACAGAAAGCTATCTCCAAGCACTCGACCGGGCCTTTGC
TGCTCTGGGCATCCGCTTACAGGACGAGAAGCCAACTGTGGGCCTGGGAGTGGATGGGGCCAACATCACCGCCAGCTTGCGCGCCAGCATGTTC
ATGACCATCCGCAAGACGCTTCCGTGGCTGCTGTGCTTGCCCTTCATGGTGCACCGGCCCCACCTGGAGATCCTGGATGCCATCAGTGGGAAAG
AGCTGCCCTGCCTAGAGGAGCTGGAGAACAACTTGAAACAGCTCCTCAGTTTCTACCGCTACTCACCACGCCTCATGTGTGAGCTGCGCTCCAC
GGCCTCCACCCTGTGCGAGGAGACCGAATTCCTGGGGGACATCCGTGCCGTGAGGTGGATCATTGGTGAGCAGAATGTCTTGAACGCCCTTATC
AAGGACTACCTGGAAGTGGTGGCTCACCTGAAGGATGTCAGCAGCCAGACACAGCGGGCTGACGCCTCAGCCATAGCCCTGGCCCTGCTGCAGT
TCCTCATGGACTACCAATCCATGAAGCTCATCTACTTCCTCCTGGACGTGATCGCTGTGCTGTCACGCCTGGCCTACATCTTCCAGGGCGAGTA
CCTCCTGGTGTCACAGGTGGATGACAAGATCGAGGAGGCAATCCAGGAGATCAGCCGGCTTGCGGACTCCCCGGGAGAGTACCTGCAGGAATTC
GAGGAGAATTTCCGAGAGAGTTTCAATGGGATCGCCGTGAAGAACCTCAGGGTGGCAGAAGCCAAGTTCCAGTCCATCAGAGAGAAGATCTGCC
AGAAGACACAGGTCATTCTGGCTCAGAGGTTTGACTCCCGAAGCCGGGTCTTTGTGAAGGCCTGCCAGGTGTTCGACTTAGCGGCCTGGCCCAG
GAACAGCGAGGAGCTCCTGAGCTTTGGCAAGGAGGACATGGTTCAGATCTTCGACCACCTGGAAGCCATTCCTGCCTTCTCCCGGGATGTGTGC
CGCGAAGGGACGGACCCCCGGGGGAGCCTGCTGATGGAGTGGAGAGACCTCAAGGCTGATTACTACACCAAAAATGGCTTCAAAGACCTCCTCA
GCCACATCTGCAAGTACAAGCAGAGGTTTCCGCTCTTGAACAAGATCATTCAGGTCCTTAAAGTTCTCCCCACCTCCACAGCCTGCTGCGAGAA
AGGCCGGAGCGCCCTCCAGCGGGTCCGCAAAAACCACCGCTCCCGCCTGACCCTGGAGCAGCTCAGTGACCTGTTGACAATTGCTGTGAACGGA
CCGCCCATCGCCAACTTTGATGCCAAGCGAGCCCTGGACAGCTGGTTTGAGGAGAAGTCTGGCAATAGCTACACACTGTCAGCCGAGGTCCTCA
GTAGGATGTCTGCCTTGGAGCAGAAGCCCATGCTGCATGTGGTGGACCATGGCTCTGAGTTCTACCCAGACATGTAGGGGGCCTGAGCTCCAGA
GGCCACAAAGCCGGTGAATATTTTTTTAATCTATACTCATAAGCTTTGATATATTATATAAATATATATTATATTATATATACATATATATATA
TATATATATAAACCTACACTGAAATTTTTTTAAAGACTAAGTTAATTATGTCCACCAGAAGCCACTGGCAGCTTTCAGAAAGGAGCAATGTTGG
CAACTGACTCTTGTCTCCGCACTTGGCAGCTGCAGCCTCCACGGCAACTCATCCTCACTTACACGAGCGTGTTCTGGGGCCACACGTGTTCCCA
CCAAACAGTCAAAGCGGCATAAGCTAAATGATGATTCTCTTGTCACTTTTAGGTGGCTTCTTCTGTTTCTGTTCTCATGTGTTGGTAGAGGGCT
GGGGGTTGGGTGCCTGCCTTCTCCTTTTTTGGTATGGTTTTGTGGCGGGGGCGGGGGGGGCGGTGAAACTTTGTGGTCTCCTCATTGGCATCCT
GGTCCAGTTACATTACATCTGGGTTTCCATGAGGGTGAATTGGATTTCTGGACTAGACTATCTGGTCTTTCTCCCCTCACCTGACTTCAGCTTG
GGAGATCTTTCTTTGTTCATTTTCTGATTGATTCCTTCCCTTGTCCCACCGTCAAATGGTCAAATGCATCAACCTCCCTCCGCTGTGAGCCAGC
TGCGTTCCCCTCCCACCCCCCCCCATGTCGCCGCTCCCCTGTGTCACCTCTGCCAACCCTAGGTTGTCAGTTCCCTTCTTCAGGCCTCTGGAGT
TTAAAGATATGAGCTGTTACACTGAACGTAATTTTTTTTTCACATACGAGCTTGAAACTTGGGAGTGGAGGGAGGGTAAGCTTTATGCCTCCCC
CTTTCCAGCCGCTGGAGGATAAAGTATATACTTCCATTCATATCTTGGGCTGTTGGGGAGACAATGAGCCACGAGATCTAAACTCCCAACAGGA
CCATGATTCTTTTGGCTAAATACAGGTAAAATGAAAAGTGTATATTCAACTTTCACATTGTTTTCATCTTTATAATTATGGGATGTGTTGTGGG
ATTTGTGTTTCTTTTCATAACCAAGGAGCAGCTGTTTTGTAGTTCAAGGGCAGAGCAATCAACGAAAGTCATGCCCTGGGCTACCCAGAACTCA
GAGAGTGCCTAGAGAGGTTTTTCCTTTTGCAAAAGGCTGATAATGTGTTCCTCAGCCTTGCAGGAAGGTCACAGGACACCAGGCAGTGCTTCTC
TGCCTGTGAACTCTTCTTATTTTGAGTAGAGTTGAGATGTCCTATAGAATAGGAGAAAACAGGAAATGAGGTGGGATTCTCAAGGTACAAGCAT
GTATACATACCTACACACACACACACACACACACACACACACACACACCACATGCACATACATGCACACACACCACATGCACACATACACACAC
AACACATGCACATACATGCACACACATGCACACACACCACTCACACACATACCACATGCACATACATGCACACACACCACATGCACACACACAC
ACACACCACATGCACATACATGCACACACACCACACACACTTTGCAACTACATATAAGCTTTAGAAATGCCTCTCGCCTCCCCCCATAGCGGGG
AAGAGAGTGGTTTAAACTAGAGGAGTCTGACCAATGTTCATTTATCTACCAGTATACCTCGCAAAGAGTTTTCAGAAATGTGCATGAGCTGTTA
AAAACTTCTCTCTAATTTCCACATCTGATCTGTGTAGTCTGTGCAGATCTCAGAATGTGGATACAGGGTTGTCTTGGGGAAGGGAGGAAGGTCT
GAAGGACCTGCCCATGGTCAGTTCTGCAGAGAACTGTCCCTCTCCTTCAGTGGAGTATTGGGGGTTCAACCACAAGGAAAGGCCCCCGTACCCT
CAACCTCCCTGGTGGGGCCTGCAGCTATGGTAGCTTCCTTCCTTCTGGGTCTGTCTCACCTCTGAGGCCTAACAGAAATGTCATTAACACTGAA
CACGTTGCTACCCTTTCCTGGTTCTGCAGTGTGTCCGTCCTCCCTCAGAGCCTCCCTTTGCCTCCATGGTCTTGGTGCTAAGATAAATTTAGAA
TCATTGCTGCTAGCCAATAGCTTTTCATGATATAAATATTATATATATATATTATATATTATTTTTGAAATGTTTTTGTTTTCAACAGTGATGT
AGCATGTTAAAACAAGAAACAAAACTGAACAAACGAAAATCTGGGTCTTTCCAACTGTCCCACTGCCAGGTCTCTTAATGCTGCCTTCTTCAAC
AAGTTGATACACCCCACCTGGTGACTTTCACCCCTCATCTCACTCTGGTCCCCATCTTAACCCCTCACCCTCTGAGCAGGGAGAGTTAGCAGGA
ACTAGGTAGGAAAACTCAAAGCCCCTTGCAGAGGCCTGGGAATCCCCCAGCCTCAGCACCCTTGCCCACGCTGAGTTTGTGTCTCTCTTCAGCT
GCCCCTCCTACCCCGGCCCAAGCCTAGCTGGAGCCACACGGGCAGCTGTTGCGAAGCACAAGGTCTCCACTTGCCTCGCTCCTGCCTCAGGACC
CCATCTCCTCACAGACACAGACTTTGGTCTGGGACAATTTTATTGAAAATTGAATAGGCCGAGGTGGGTTCAGAGTAGGGATGGGTGGCAAGGC
TGGTCTCTCATGGGCAGCCTGTAGGCATAAACCGTGGCCTGGGCCCTTCCTTCTTGCCAACAGTTGCCTTACATCTGTCCTTCCCAGTGCATGG
GGCTCCCTCACCCAGGAAGGGACCTCTCCCAGATGGTCCTAAGCCAGACATGTAAAACCAGGACTGACCGTCCCCTCCCAAGGAGCTATAGCTG
AGTGAACTCAGGTCCTCATCCATAGAGACAGGTTCTGCCAGGCCAAGCCATTGTTACCTGAGTCTCCTCAAGATACCCAGGATCAGTGGACCTG
TCTTGAGGCCTAGAAGAATGAGCTGTTCTGGCAGTATCATGTGGCTTCAGAGTCACCCATTTCCTTGGTCCTCTGTCTCCTCAACCCCACCTCT
GTCCTCTGCTGCAATGACCTCTGCCTCAGAGCCACTGAGAAGCTGAGCTCTACACGTGTCTTCTGCTCAGTCTCCGAAACTCTGGGTGCTGGTG
ACTGTGCAGCCAAGCAGGGCTGTGACAGATGCACCATGGGCCACTTTGCCTCGGGATATTGAATGACTAAGGGACACTGAGGTTGAACCCCTTG
TAGTCAGCTTGGATGTTCCCTGGGAGGGTCAGGGCTTCTGCCCAGTGAGTTCCATTTGTGATGCCCTGTGATTTCAGACCTGTTCTTATCCTCC
TCTTGGAGAAGGGCTTGGGTCTGCCCACCCTTCATGTCTGGCCCGAGAACCATATGCCTGGCTCACCCCATATCTTGGCTGGGTTAACAGACTG
TGGGAAGGTAGGGCTTAGAGTGTATATGCAGTCATTTCTGATGGGACAGGGACTGTAATTCAGTCTCTGGAGTAGAACTGGTAGTCACGGGCAT
AGAATGAAATGTATCTGAGGGGCTGAGACTTATTCTCTCTGGGCTGGTAGTGGCTTTCATTGGTTCACGTTGGTGAGAGTAAGAAGTGAGGCAG
TGTTGAAGACTGAGGCTTGTGGCCATTGTGCCAAATGGGTACCTTGCTGAAACACAGGGTTACAGTGTGCCCCCCCCCCCAGCAAAGGAAAACA
ATGACCACTGAGACAGGTGGACCACTCAGCTGCGTGGGCAGCTCCTGCCCAGTTAGAATCCCTCCAGAGACATCAGAGTGCGGGCATCAGAGGT
GCCAGAGCTATTGTTTTATAATGTCTGCTTGTGGCATCTCCAGCTGCCTTACCCCCTCCTCACTGGGGAAGGAGAGTGCACTCAGCTCCGGGAC
TCTGTGTAGTTAAGTTCTGCCTGGAGCCCTGAATCTGAGGTCCCAGAGCTTCTGCCCTCTGCTCTGGCTGTGTGTTCCCAGCCTGCGTTGTTTT
GCTGAGGTTTCTGTTTGACTTGTTCAAGTCAGATCTTTGATTTTGAGCAAGGACCTTGGAGTGACTTGAAGCACCTTTTAAATGCCTTTCATTT
GTGTGAAGGGGGAGGGGATGGGGGAAGGTGTGGAGAGGGTGGGTGTGGCCGAAGAGAGGAAGAAATGGACTTTAAGATGTTACCCAGCCTGAGC
TGGCGGTCCTTTGCTGCCCCCCTGGAGCCAGGTGATGGGGGCCAGGAGATGCCGGATGGGTTGAAGTCTTCTAGAACACTCCTTTGTTTTTTCA
TTTCTGCCCCCTTGGAAGGCATCCCAACCTGTTTTCAGTGGATAGTCTGGGTTAAATTCTAACCCGGATTTGTGCTTTTTTTTTTCTTTTTGTG
ATCCATGCACCTCATTTGGCTGCAGGCCCCAGGTTCACTGGAGCTCATGTGTGCCCTGGATCTCCTCTAAGAAGCAGCCCAGCCAGACTGGGCA
TGCAACGGAAGCAGGGAGAACAGGGCCTCTGGAGTGAGGAAAGGAGGGCAGAGCCGACAGTTAGGGTGGGATCACCCCTTTACACACTGCCCTG
GATTCCAGCCTGACGGTTCCTCCCTTCTTTGAGGGGGTGGGGAATGTGTGCCGGGTGTGCATGGTCCTCCTACTCGGAGGAATGCTCTTTTCTA
CTCCTCTGTTAGTCAGGGTCACTTTAAGAGTCTTTGAGGTAATCTTTGCATGTTAGTGAGAGGGAGAAAAGTGCTCTCTGGAGAGAAAAATAGT
CTGGCCCTCCTCTCCCATCTAGCACTCTCTCCCCTCCCCCCATCCCTTCAAATGGTATCACCCTAGAAATACCTATGCAATGCAGTCTCCCCAG
GAGGCTGTGCATGGAGGCAGGGGTATGTGCAGTGTATCATACTGATGCTAAGGCATATCAATTGTATATACGGACATATACAGTACGTATACAT
GCAGAGTAAGAGAGTAAATCACGTCTATATATCTATAAATAATATCCTATATATTTATACATTTTTATGTTTTAAATAGATATAAAAATAGTCT
ATAGAGCTGGAAGGGTGGCGGGGAAGCCTGGTGCTGGGTTGGGCTTGGGGGGAGGAGACAGGGCTCTGGAGAGGAGGTGGGGATAATGCGCATC
AGAATGGGTTGCTAGCCACTGAGATGGGGGCATCTTTCTTGGGGTGTTGGAGAGGAGGGTGGCTGAGCCTGGGCTCTCTCTCCATGCACTTTCT
CTCCTTTCCTGCCAGCAAAGGCTGAGATTGGAAGGAGATAGCCCTGAGCTGCATGCAACTGCAGTACCCTACCTCCCAGCACCACCAGCCTGCA
CTTAGCTGCTCACTGCTGCTTAGCAGTAGGGGGTTGGACTTGGTCCCTGACCCCCAGTGAAGGGACCTCTTGCACAAACTCCCCAATACCCGCC
CCTATCAGGAGTGAGCAGCATTGATGGCAACGGGAAAGGGGCACCCGGGACAAACCGTGGTCCAGAACGATGTCAGTCCCATTTCCCAATGCCT
CTGCCTACCCCCTGGAGGCCAAGCCTCCCCTGCCTACCCCCTGGAGGCCAAGCCTCTGACACACAGTCCTGGCCTCCTTCACCCATTTAACCAC
TGTCTCTAGAGCACTGGGAGCTGGGGAGGGCGGCGTGCACTTCAGGTGCACTTGATGCCTTTCCCCATCCAAAGGCCCCACCTGGTGGAAGAGA
TATATCTGCACACTGACATTTTCAATGGCAACTCAAATTCAGGAGCAGGGCCTTGGCCCACCCGACTCTGCACAGCTGCAGCAGCTGCTGGCTG
TACTCAGGACAATGTTGTCCCCTCTTCCCAAACCAACCAAGCAGCCCAACCACCTCCGCTGTGTCAGCAGGTTTAGGTTTAGTTCACAAACACT
GTTCTCATTCTGTGACCAGATACCCAAACCCACTCTTTCTAGGGAGGGTATCTTCTTCAGCTTGTCCCCAGGCAACACCTCCCCCCTCAAGCTG
GGTAGGGATGTCTGTGGTCCCTGGAAGTCCAGTCAGCTTGCTCCCAGCATCCTCTATTCCCTTCTCTCTCCTGGAAGTTGACAGGGTCTGATGA
CAGATGCCCTCCCTGCTCTCGAGGAGCAGCCTGCCTTTGGGCTCCTCAGACACACTCATTTCCCATACCGAGCCTTCGTAGGTCTCTCCCTGCT
TCTCCTCACCATCCTACCAACGGACCTCAGTCTCCAGCAGACCAGTCCCATACCACAGTTGAGACCAGGTCTGCTCTGCCAATGCCCGTCTCCT
TGAGCCACAGCTAGCTGCCAACTGGGCTGAACACCTCCTAGTGCCTGGCTCAGTAGAGTTCAGGACCAATGGCCCAAGCCTTCCTCCCACTACA
ATGGGTCAGAGCTTGGCCAGGGGATAGGCATTCCCAGTAGCAATCCCACAATGCACTGTACCTCAGAGAGAGAGAGCATGCCACAGGGCATCTA
GTGGACCAGTCTCCATGCAGAGGAGGCAACGGCTCACAATACTGCTCACGAGAAGACAAAGGCCAGGCTGTCCTAGGGCATCCCTCAGTCTACC
CTTTGGCCTCTCTGGAAGAACCAGTAATCTTTTTCAGCCCTGATCTCTCTCCTCCTCTCCACCCCCAAGCTGCTGAACCATTTTTCATGTCAAT
CACAAAGAAAAAAAAAAGTGGGGGTGGGGGGGGACTCCTAGGAGCCTATTACCACATACATATTAATATGTTAATACTTTCTTTATTAAAAACA
ATTCTCAATGTTATTATTTGGCAGACTGTGCTTTCTAGTACCTGTGTGTTGGGGCTAGAACACTGGATACAAATAGAGCTATGCTGGTTTATAA
TCTGTATGCAGCGTCTTTCCTTTTGTCACATTATCCTTGTAATGTAAGAAGATTATTAATAAAAGCATTTAAATTGCCACCCCAAGCTTGGCTC
CTTGCTTCTGTGACAGGCCTCCATAAGTCGTGCATACCCTGTCAAAGGGCTCAGGTGTGGGTTTGGAAGGATCACAGGTCCTCTTTAGCTGGTC
TCTGCCTTCTCCCACTATGGCCTCATTTCCCACTGCAGTCCAATTCTGGCAGGAAGCCCCTGAGCATGTCAGGTGGGCCAAAGAAGATATGACT
GAAACCGTGTTAAACACCTGCCCCCATGACACTGGCCCTGCAAGAATCGCTGTGGGGAAGCATCCAGGGCCTCTGCAAAGCCAATGGCTCCAGC
TCTCTTGCTTACCTGCATTTGGTTGCTCTCCATGAGCGGGCCTGATCCCCACTTAGAAAAGTCTTAGAACTTTGTTGATAGCACTAGGGACAGA
GGGTGACAAGCAAGATCTTTTCCACATTTTTTAAGGAAGGTATTTGACAGGAATATAGGCAGTATTGTGAATGTAGTCAAATACCTCTAAGTGC
AGGTTTATCCTCTTGAGATGCTGAGGCCTCTGCTCTCAGTCCCTATGCCCCTAACTTCCTCATAGTGGTCTTCCCTCTTCCTGTCTCCTTACTT
CTGCCTTCTTATCATTAGACATCTGTCGTTCAGCCAAGAAGCACCATCACCTTGACAGTGGCCACCAAGCAGCTGTTGTGTGATACTATTTATG
GAGAAGTGTGAACAAAAGTCTTCTCAATCCAGATATAGAACCAATAGCAGACCAAAGTAAGGGTACTATCAAAGTCCAACTTGGTGAGCCAATG
AGCTTATTGGGGCTACTTGGAGAAATATGAGTGAGAGCTTTCTTACAACAATATGGGTAACTCAAAGGCTGCTACATTACCAAAAGCCCATTCC
AAGGTGAGTAGTCTCATGAAAACCAGAACCCTGGCCATCTCTTGGTAGCTTGCATCAACTCAACAAGTAGGACAGCCTACTCTTATAATATAAC
ATTGGAGAGGGAGGAGCTTTAGAACTCTGATTAAGTTTCAGGGACTTCCTGAGACTTATGAGTTGTTTATTTCCAGAGTCTTAAGAAGCCTCTC
TTTAGAACCTCTCAAGTCCTAATGAGACTCTATTCAGGACGCATTGTTTCAGTTGCTACACAGTACTCTACCCCTTCCTGCCAGCTCTTTCTGC
TCTCCTTCCATTGT
MOUSE SEQUENCE - mRNA
TCCCCAGGACAGAATGACTGAGAACATGAAGGAGTGCTTGGCCCACACCAAGGCGGCTGTAGGGGACATGGTGACAGTAGTGAAGACAGAGGTC
TGCTCACCACTCCGGGACCAGGAGTATGGCCAGCCCTGCTCTCGGAGACTGGAGCCATCTTCCATGGAAGTTGAACCCAAGAAACTGAAGGGAA
AGCGTGACCTCATTGTGACCAAAAGCTTCCAACAAGTGGACTTCTGGTTCTGTGAGTCCTGCCAAGAGTACTTCGTGGATGAATGCCCGAACCA
CGGTCCCCCCGTGTTTGTGTCTGACACGCCAGTGCCTGTGGGCATCCCAGATCGGGCAGCCCTCACCATTCCTCAGGGCATGGAGGTGGTAAAG
GATGCTGGCGGGGAGAGCGACGTGCGGTGTATAAACGAGGTCATCCCCAAGGGCCACATCTTTGGCCCCTATGAGGGGCAGATCTCTACCCAAG
ACAAGTCAGCTGGCTTCTTCTCATGGCTGATTGTGGACAAGAACAACCGCTACAAGTCCATAGATGGGTCGGATGAGACCAAGGCCAACTGGAT
GAGGTACGTGGTCATTTCCCGGGAGGAGAGGGAGCAGAACCTCCTGGCGTTCCAGCACAGCGAGCGCATCTACTTCCGGGCATGCCGAGACATC
CGGCCAGGAGAGCGGCTGCGGGTCTGGTACAGCGAGGACTACATGAAGCGCCTGCATAGCATGTCCCAGGAAACCATCCACCGCAACTTAGCCC
GGGGAGAGAAGAGGTTGCAGAGAGAGAAGGCTGAGCAAGCCCTGGAAAACCCAGAAGACCTAAGGGGTCCCACTCAGTTCCCTGTGTTGAAGCA
GGGCAGAAGTCCTTACAAGCGCAGCTTTGATGAGGGAGACATACACCCTCAGGCAAAGAAGAAGAAAATAGATCTCATCTTCAAGGATGTGCTA
GAGGCCTCACTGGAGTCTGGTAACGTGGAAGCCCGCCAGCTGGCCCTGAGCACCTCCCTGGTCATCCGGAAAGTCCCCAAGTACCAGGACGATG
ACTATGGTAGAGCTGCCCTGACGCAAGGCATCTGCAGGACCCCGGGGGAAGGGGACTGGAAGGTCCCTCAAAGGGTGGCCAAAGAGCTGGGCCC
ACTGGAAGATGAAGAGGAGGAGCCTACATCATTCAAGGCTGACAGCCCTGCAGAGGCCTCTCTGGCATCTGACCCCCATGAGCTGCCTACCACC
TCCTTTTGTCCTAACTGCATTCGCCTGAAAAAGAAAGTGCGGGAATTGCAGGCGGAGCTAGACATGCTTAAGTCTGGAAAGCTGCCTGAGCCCT
CCCTTCTGCCACCACAGGTGCTTGAGCTCCCAGAGTTCTCAGATCCTGCAG
MOUSE SEQUENCE - CODING
ATGACTGAGAACATGAAGGAGTGCTTGGCCCACACCAAGGCGGCTGTAGGGGACATGGTGACAGTAGTGAAGACAGAGGTCTGCTCACCACTCC
GGGACCAGGAGTATGGCCAGCCCTGCTCTCGGAGACTGGAGCCATCTTCCATGGAAGTTGAACCCAAGAAACTGAAGGGAAAGCGTGACCTCAT
TGTGACCAAAAGCTTCCAACAAGTGGACTTCTGGTTCTGTGAGTCCTGCCAAGAGTACTTCGTGGATGAATGCCCGAACCACGGTCCCCCCGTG
TTTGTGTCTGACACGCCAGTGCCTGTGGGCATCCCAGATCGGGCAGCCCTCACCATTCCTCAGGGCATGGAGGTGGTAAAGGATGCTGGCGGGG
AGAGCGACGTGCGGTGTATAAACGAGGTCATCCCCAAGGGCCACATCTTTGGCCCCTATGAGGGGCAGATCTCTACCCAAGACAAGTCAGCTGG
CTTCTTCTCATGGCTGATTGTGGACAAGAACAACCGCTACAAGTCCATAGATGGGTCGGATGAGACCAAGGCCAACTGGATGAGGTACGTGGTC
ATTTCCCGGGAGGAGAGGGAGCAGAACCTCCTGGCGTTCCAGCACAGCGAGCGCATCTACTTCCGGGCATGCCGAGACATCCGGCCAGGAGAGC
GGCTGCGGGTCTGGTACAGCGAGGACTACATGAAGCGCCTGCATAGCATGTCCCAGGAAACCATCCACCGCAACTTAGCCCGGGGAGAGAAGAG
GTTGCAGAGAGAGAAGGCTGAGCAAGCCCTGGAAAACCCAGAAGACCTAAGGGGTCCCACTCAGTTCCCTGTGTTGAAGCAGGGCAGAAGTCCT
TACAAGCGCAGCTTTGATGAGGGAGACATACACCCTCAGGCAAAGAAGAAGAAAATAGATCTCATCTTCAAGGATGTGCTAGAGGCCTCACTGG
AGTCTGGTAACGTGGAAGCCCGCCAGCTGGCCCTGAGCACCTCCCTGGTCATCCGGAAAGTCCCCAAGTACCAGGACGATGACTATGGTAGAGC
TGCCCTGACGCAAGGCATCTGCAGGACCCCGGGGGAAGGGGACTGGAAGGTCCCTCAAAGGGTGGCCAAAGAGCTGGGCCCACTGGAAGATGAA
GAGGAGGAGCCTACATCATTCAAGGCTGACAGCCCTGCAGAGGCCTCTCTGGCATCTGACCCCCATGAGCTGCCTACCACCTCCTTTTGTCCTA
ACTGCATTCGCCTGAAAAAGAAAGTGCGGGAATTGCAGGCGGAGCTAGACATGCTTAAGTCTGGAAAGCTGCCTGAGCCCTCCCTTCTGCCACC
ACAGGTGCTTGAGCTCCCAGAGTTCTCAGATCCTGCAGGTAAGTTCCTCAGGATGAGGTTGCTGTTAAAGGGAAGAGTGGATTCTGCAGATGAA
TCTGTCTCCAACGACGTGATGACGGCAACCGATGAACCCTCCAAGATGTCCTCTGCCACAGGACGCCGCATCCGGCGCTTCAAGCAGGAGTGGC
TGAAAAAGTTCTGGTTCCTACGGTACTCCCCGACCCTGAATGAGATGTGGTGTCACGTGTGCCGCCAGTACACGGTTCAGTCCTCACGCACTTC
CGCTTTCATCATCGGCTCCAAGCAGTTTAAGATCCACACCATCAAGCTGCACAGCCAGAGCAACCTGCACAAGAAATGCCTGCAGCTGTACAAA
CTGCGCATGCACCCGGAGAAGACTGAGGAGATGTGCCGCAACATGACCCTGCTCTTCAACACCGCCTACCACCTGGCGCTGGAGGGGAGGCCCT
ACCTCGACTTCCGGCCCCTGGCAGAGCTGCTGCGGAAGTGCGAGCTCAAGGTGGTGGACCAGTATATGAATGAGGGGGACTGCCAGATCCTTAT
CCATCACATCGCCCGGGCACTGAGAGAAGACCTGGTGGAGCGCATCCGTCAGTCGCCCTGCCTCAGCATCATCCTGGATGGGCAGAGTGACGAC
CTTCTGGCTGACACAGTGGCCGTCTATGTGCAGTATACCAGCAGTGATGGACCCCCAGCCACAGAATTCCTATCCCTGCAGGAGCTAGGCTTCT
CCAGCACAGAAAGCTATCTCCAAGCACTCGACCGGGCCTTTGCTGCTCTGGGCATCCGCTTACAGGACGAGAAGCCAACTGTGGGCCTGGGAGT
GGATGGGGCCAACATCACCGCCAGCTTGCGCGCCAGCATGTTCATGACCATCCGCAAGACGCTTCCGTGGCTGCTGTGCTTGCCCTTCATGGTG
CACCGGCCCCACCTGGAGATCCTGGATGCCATCAGTGGGAAAGAGCTGCCCTGCCTAGAGGAGCTGGAGAACAACTTGAAACAGCTCCTCAGTT
TCTACCGCTACTCACCACGCCTCATGTGTGAGCTGCGCTCCACGGCCTCCACCCTGTGCGAGGAGACCGAATTCCTGGGGGACATCCGTGCCGT
GAGGTGGATCATTGGTGAGCAGAATGTCTTGAACGCCCTTATCAAGGACTACCTGGAAGTGGTGGCTCACCTGAAGGATGTCAGCAGCCAGACA
CAGCGGGCTGACGCCTCAGCCATAGCCCTGGCCCTGCTGCAGTTCCTCATGGACTACCAATCCATGAAGCTCATCTACTTCCTCCTGGACGTGA
TCGCTGTGCTGTCACGCCTGGCCTACATCTTCCAGGGCGAGTACCTCCTGGTGTCACAGGTGGATGACAAGATCGAGGAGGCAATCCAGGAGAT
CAGCCGGCTTGCGGACTCCCCGGGAGAGTACCTGCAGGAATTCGAGGAGAATTTCCGAGAGAGTTTCAATGGGATCGCCGTGAAGAACCTCAGG
GTGGCAGAAGCCAAGTTCCAGTCCATCAGAGAGAAGATCTGCCAGAAGACACAGGTCATTCTGGCTCAGAGGTTTGACTCCCGAAGCCGGGTCT
TTGTGAAGGCCTGCCAGGTGTTCGACTTAGCGGCCTGGCCCAGGAACAGCGAGGAGCTCCTGAGCTTTGGCAAGGAGGACATGGTTCAGATCTT
CGACCACCTGGAAGCCATTCCTGCCTTCTCCCGGGATGTGTGCCGCGAAGGGACGGACCCCCGGGGGAGCCTGCTGATGGAGTGGAGAGACCTC
AAGGCTGATTACTACACCAAAAATGGCTTCAAAGACCTCCTCAGCCACATCTGCAAGTACAAGCAGAGGTTTCCGCTCTTGAACAAGATCATTC
AGGTCCTTAAAGTTCTCCCCACCTCCACAGCCTGCTGCGAGAAAGGCCGGAGCGCCCTCCAGCGGGTCCGCAAAAACCACCGCTCCCGCCTGAC
CCTGGAGCAGCTCAGTGACCTGTTGACAATTGCTGTGAACGGACCGCCCATCGCCAACTTTGATGCCAAGCGAGCCCTGGACAGCTGGTTTGAG
GAGAAGTCTGGCAATAGCTACACACTGTCAGCCGAGGTCCTCAGTAGGATGTCTGCCTTGGAGCAGAAGCCCATGCTGCATGTGGTGGACCATG
GCTCTGAGTTCTACCCAGACATGTAG
HUMAN SEQUENCE - GENOMIC
AACCCTGTTGCAGACAGGCCCAGGCAATAAAGCAGTGTAAGAGGAAGTGCAGAGGTAGCGTGGATTTCAGGACCTGTTGGTTCAGCACCTCAAC
CGTGTCAGTTACAGTTTCTGTCTCTGAGATGGTTCCCAACACCAGCCCCTTCTTGGATTCTCCAACCCAACTGGGTGTCCAACAATTCAATTCA
ATTCAATTCTGTAACTATCTAGAGCTGGTGCAGACCCCACAAGATAAGAGCTCAGTTACACAAGACTGCCCTCAATTCAGACACTGGTCATAAG
TCCCAGGGGACTTGTACCTCTGACCAACATAAATCCAGGGCTCCTACAAACCCCTTTCTCAGGTGTGATAATTCACTTGAATAACTCACAAAGC
TCAGGAAAGTGATTTACTTACTATTACTGGTTTACATAAAGGCTACAACTCAGGAACGGCCAGATGGAAGAGCTGTGCAGGGCAAGGTGTAGAG
GAAGGGACCTGGAGCTTCCATGTTCTCGCTGGACCCTCCACCCTTCCAGCACCTTGCTGTGTTCACTAATCCAGAAGTTTTCTAAATCTCCTTC
AAGAGTCTTCACAGAGCTTCATCTCCAGCCCCTCTTCTTGTTGCCAGAGGCCAGTGGTTGGGATTGAAAGTTCCAACCTTCCAATCACGTGTTC
TTTCTGGTGACTCAGCCTCATCCTGAAGCTATCTGGGGGGTCCCACCCTCAGTCATCTCATTAGCATAAACTCAGGTATGATCCGGTGGGGCTC
CTTATGAAGCACCAAAGACACTCCTATAACCCAGGAAATTCCAAGGTTTTAGGAGCTCTGTTTTATTAGCCAGGAACCAGGGACAAAGACCAAA
TACAGTCAACCCCTCCTATCCATGGGCATCCATGGATTCAACCAGCCATGGCTCAACAGACTTTCTCCTTGTGATTATTCCCTAAACGATACAG
TATAGCAGTGATTTACATAGCATTAACATTGTATAAGGCATTATAAGTAATCTAGAGATGATTTAAAGTATATGGGAGGATATCCAAAGGTTAT
GTGCATATACTATGCTATTTTATATCCAGGACTTGAGCATCCTTGGACTTTGGTATCAGAGAGGGTCCTGGAACCAATTCCCCTCAGATACCAG
GGTGCAAATGAATGTGTGTTTCTTGTCCTACCACCCTGTCCCCCTGCTCTGAGGAACTCGCTTCATGCTGAAGATGCTTCCTGCTGGGGTCCTC
TGCAGGACATGGCTCCCATTTGCAACAATGGCTGTGTGCTTTCTCCTTCTAGTCATGAAATAAAAGCCCTTCCCTTCCCTTTGATTTGGTCAAA
ATAAGTCAAATGTCTGATCTTGGAGGGAAATGTCATGTGCTGATTAAAGGCCAGGTTCCTGATCCAGTCATTGGGGAGGGAGGTGGCATTGCTA
GAATTGGCTTAGATGAACCTGAGCCCAGCCCTGGTGCTGGGCAAAGGCTCGGTGTCTCTGAATCTCACTTAGTTGGTCCTATAAGGGAGGTGTG
GCACCTGAACAAAGTCCAACTTCCAGTGGGGAGTAATGAGGTAACGGATACCATAGAAGCCACCAGCAGGATCCACTGTGAGAAGCAAACACAT
AAAAAAGTTCTGGCTGGGCGCGGTAGCTCACGCCTGTAATCCCAGCACTTTAGGAGGCTGAGGCAGGTGAATCACCTGAGGTCAGGAGTTCAAG
ACCAACCTGGTCAACATGATGAAACCCTGTCTCTACTAAAAATCCAAAAATTAGCTGGGCGTGGTGGCGCACGGCTGTAATCCCAGCTACTCGG
GAGGCTGAGGCAGGAGAATCGCTTGAACCCGGGAGGCGGAGGTTGCAGTGAGCCGAGATCACGCCACTGCATTCCAGCCTGGACAACAAGAGAG
AAACACCGCGTCAAAAAAAAAAAAAAAAAAAAGTTCTGCGGGAGGGACCGCCTTGGGAGAATGTGTTCCACCAGCCCTGGCCAGCTATACCCAT
GATGCTTAGTGGCGACTGCCCTGTGGGTAAAAGCTCAAAACCTCATTTTCCATGACCCCAGCAGGAGCCTCCACTGGCTGGACCCCAGTTCCTG
CGGCTGCAAAATCAGGGACTGGACAGGGTTAGAGGTCCCCATATGGGAGTTCCTTGCCCTCAGGAGGCTCCAGCAGATGGTTTTTCTTTATGTT
TATTAACATATATATATTCAGAAAAGTGCACATATTGTAAAAGCTGGCTGAGTATATTTCCACAAACTTATTATACCCATGGGATGAGCACCCA
GATGAAGAAATAGAACATGGCCAGAATCTTAGAACTCCTTTTCCTGCCTTTTTCCAGTTATTCCCAAGAATAACCACAAACTTCACCTCTAACA
CCATAGTTGTGGCTGGTTGGTTTTTTTTGGTAACTCTATTTAAATGGAATCATACAGTATGAGTCTGTACTCTTTGGGGGTCTCCTTTCTTTTC
TTGGCATTATCACTCATCCAGATTATAGCATGTAGTTGTAGTTTGTCTATTCTCATTTCTGCATAGTATTCTATCCACTGAATTTGGTGAATAT
TGTACAGTATCCTATGATCAGGTAGGACAAGTGAACAAACATGTCAAATTTCTTTGCTTTTGCTTAGGATGACGGCCGCTAACCAGTGTATTAA
CTCTGCTTATCATGCACCCTGTGTGTATACAAAGAACTAGGAAGATGAATTAATTATTACCTAATGCATGCAGTCTTTTTAGTAGACATGATCT
TCCAAAATGGGAATCCTAAACAAAAATAAAAACAGGTATGCTTCGGGTAGAGATATGGGGGTCCTTACCGATTGATTGATTCATTGAGTCATTG
ATTCATTCGTTCATCAAATATGCATCAAGCGCCACTTGTGTGCCTGATACTCAGTCTCTAATCTGGATTCCTCCTTTCTCCTAACCAATTGGCA
GGATCTCAGCATCTAATGACAAGTGGGTAAAGGTCATTGGAATTATCTTCTTTAGCAATTTCTAAACTAAATGATTATGAAGAGGTCCTCTAAA
GAATATTTAAGTGACTATTTTTCAGAAGCAAAAAGAAGAAAACAGAAATACACAACCCTCCTCCTGAACTAACCACTAGTGATACCTTAAAATA
TATCCTCCCAGACCCATGTGTACAGATCTGCATCATTATGTATAGACTTTAAGTGGAATTATTTTGTACACCTGTGTAGTGTGGGAAATTAAAA
GTGTTTTAATGGAATTATTTTGTATCTATCTTTTCATAGCTTTCATTAACAAAATCCTTGTTTTTTAGAGCAGTTTTAGGTTCACAGCAAAACT
GAGCAGAAACTAGAGTTCCCCATACACCCTGCCCCCACACACATCCAGCTTCCCGGTTATTAACATCCCACACCAAAGTGGAACATTTGTTACA
ATTGATCAACCTCCATTGAAACATCGTTATCAACCAAAGTCCATAGTTTACATTAGGGTTCACTCATGGTGTTGTACCTTCTGTGGGTTTTGAC
CAATGTACAATGACATGTGCCTTCCATTGTAGTATCATACAGAGTAATTTTACTGCCTAAAAATCCTCTATATTCCACCTATTTGTTCCTCTCT
CCCCGCAACCCCTGCCAACCTCTGATCTTTTTACAGCCTTCGTGATTTTGCCTTTTCCAGAATGTCATATAGTTGGAATCATACAGTATGTACC
CTTTTCAGGTTGGCATCTTTCACATAATAACTTGCATTATCATTTAGCAATATGTTGAACGTGTGTTTTCATGTCAATAAATATTCCTCTACAG
CATTGTTAATTGCTGTCTATTATTTCATTGTATGCTAATATTTCATTGTGATATAGCATAGCAATTTCCCCCATTTTGGAGGTTTAAATTCTTT
CCTATTTTTTGCTTTTAAAAATAATATTGCAGAGGACAGCTTTGTAGGTAAATCTTTGCACGCAATTTAAATACATCCTTAGGATCCATTCCTG
GATGTGGGCTTGCTCCTACAAAGGGTTTACTTACTTTGAGGATTTTGATACATGTGCCAAATTACCCTCCAGAAAGGTTACACCAATTTACACT
CCAACCAGTGGGATATGAAGTACTAATTTCCCCTACACTCTTGCCAACTCTGAATAACATCAAGATTTTTCATCTTTGCCAATTTATTAGAGGA
GAAATGATATTTATTTGTTTTAATTTGTATTTATTTGAGTATTAATGTCTTCATGGTTTTCCTTAAAGCATATTAGCCATCTATTGGTCTTTGC
AGATTGCTTGTTCATGTTTTCTTTTTATCCATTTTTTTTTCTATTGAGGTAAGCATCTTCAGAGCATATATTTATAAAAAGTATTTATTAGGGG
TATTCACTGCTTGTAAAGGCCCTCTTCTAATGTCAAACAATTTTTGGCCGGGTGTGGTAGCTCATGCCTGTAATCCCAGCACGGATCCCAGGGC
AGATCACCTGAGGTCAGGAGTTCGAGACCAGTGTGGCCAACATGGTGAAACCCTGTCTCTACTAAAAAAATACAAAAAATTAGGCAGGTGTGGT
GGCAGGTACCTGTAATCCCAGTTATTGGGGAGGCTGAGACAGGAGAATCGCTTGAACCCAGGAGGCAGAAGTTGCAATGAGCCGAGATCGTGCC
ATTGTACTCCAGCCTGATCGACAGAGCGAGACTCAATCTAAAAAAACAGAAATTTCACCACCATGTGCACATCTGATGTCTGTTATACTTTTAA
ATATTAATAAAATGAATGATAATATTTATAGGGTACTTACGATAGGTCAGGCATTATGCTGTGTTAAACAGCCCTATGAGATAGGTTCTGATAT
CAGTGCCACAGGACGGATGGGGAAACCAGGTAGGCGTGGTTAATGCAGTTTCTCAAGGTTACACAGTTTGTGAGTGGCTGTGCTGGTGTTAATT
GATTAACAAAATACATGGTGACATAAGGTTTCTATGAATTCAATAACACTTTTAAATACATTTATCTTTTGTGTTCATCACAAAAGTATTCATT
TGCAAATCTGTCTTATACATGTAAGTTTAAGGCATATATTTATGTTCTCTATAGGCATGCTTGCTGAGAATATAAATTCAATAACTCCCTATAA
TAATTCCAAGCCCTTGCCCACCTCTCACATGGTATACTGTTAAGGACTATGAGTTGGACCTTTTATTTTACAGGAAAGAGAAACTGAGGGCAAG
GCAGAAACTTTATCAGGGTCACCTAAAAAACTGACAGCACAGCCAGAACTGAGACCCAGGGCTTGGGATTCCCAGCCCAAGTCAAGGTCAGCAT
GTGGCTTGGGCCAGCACCCTAACCTCATGTCCTTTTGGTATCAATGGGAACAACAGAGATGCTGTGGTAGGTACCAGAGTAAACCTTCTAGATT
CTCATGGTGCCAGCCTCTAGGGAAACCAAGTGGGCACAGATCCCCAGTCCCCTGGCTACACTGGCTCCTGGGCATTCCAGGATCCTGGCACCCT
GCCAAGGGTGAAAACAGAGCTGCAGACTCCTGGGCTGCATTCTGCCCCCTTCTTTTGGGTTTGCAGCTCACTGCTGGGTTTGCAACAAGCCTAA
TCTCTGCATGTGCAGACTTAGAGGAGCCAGCTGAGAGGGAGCATTGCCAGCAGCTTGGAATCCTCCATGAAGCCTGCCGACCCCCCTTCCCCCA
AGACTTTTGCTGGGCAGGTAGGACTAGAGCATTCTTCAGAAAAGGCAACAGGGGCTTTACCTGGGCCAGGCCTTAGAGTGTCTGCTGAGATGGA
CTCAGTGAAGGACACCAGCACATGTTCTCCCTCTCTCTCTCCCCAGTGGAAGTTCATTTGGTCCTGCCACATCCCATCTTCTCCCTCCCTGTCC
CTATGGCCTTTATCACAAGCACAAAAGTCTGTCTATTTCAAAGTAGAGATGTCTATGGGCCACTTGAGAGTTGGAATGCACATTAGTCCTTTGA
GGCTTGATCCTGGTTCTCCGGGCAGCCAGCTCCAAGAGTAGAAAGAAATTATATGTGCATATAGTCAATAAATAGGTGTCAGATAATAAGGACT
CGGTAACTCATTATGTTTCCCTTTATGCTTTGGCTGCCAGGAGGCTCAGAATTGAATGAAGAACTCAATCACTAACCCTTTTACAACAGATTCC
CGGCATAACCAACACCCCTCTTTCTCCATGAGGCCTCTAACACTATCTCTGGTTGTTTCTTAACTGTCCTGTTTAGTATGTGTTTGTTTTATAA
AACATTGCAAATCTTTTGTGTGGTGAAGGTGGGATGTGAGAGATAGGACATAAATGCAAATGGCCCACAGATGTCTCTACTTTGAGATACAAAG
TAATACTTTGCCCACAGGGGGTGGTTTGCTGGGAATGATTTAAGACAGGAAAACGTTTATATGCCTCGGTCCTTCTCTTTCAATCTCCCTTCCC
CTCCTCATCTTCTCTTTTCTCCCTTCTCTCCTCCTTCTCTCCCTGTCTTACCCTCCGTCCTTTATTCTCTTACTATTGCCTATCCTCCCTCCAA
ACCCCTTCCGCATCCCTCTGTGCCCTCCTCGTTTATTCCTAATGGGGACCACCTCCTCCTGGCAAGTATCAGTATTTCCATTTTGCAAAAGAAG
ACACTAAGGCCCTAGAAAGTGTAAGTGACGTGCCATGGCCACACTGATTATCCTCCCCAACTTATCGTCAGAATCCGGCTCTGAGTCTCATGAG
TCTAATTCCATCCTACCCCCTAGTGCTCCATCCTACCCCCCAGTGCACCTCTGCACCCTTCAGTGCCCACCCTGCCAGCATCTCTCACTACCTG
CTCTGCCTGGCGGAGATCACTGGAAGAGAGCTCCATAGGGAATCAAACCCCCACCCTGTTTTTCCTGCTGACGTCTCTTTCAGAGTTGAGATTC
CCAATGGGACCAGTGGGACACATCAGAGGGACCAGTGAGATATGAAGGGGAACATTTGGTGAATATTTACTGAGAATATGCTGTGTGCTGGGCA
AAGTGGAGAGTCAGCCAAGCAAGGTTCTCAGGGTGCAAAATTGAAAGAGGTTCTCACTCTCAGGTGCCAGTCCTGCAAGCGTATGACCCTCAGA
GTGAAAGCCTCCTAGTACCTGGCTTGTCTTTCCCTAGTCTTGGCCCTGGTGCCAGGCACTGTTACAGGTGCTGGCAGTTCAATGGTGGCCTGCA
CCCTGTCCTCGGGGAGCTTACAGTCTATTAAGGGAAACAGATATTAATCAAGTAATCCTGCTAACAAAAGACGGTTATACATGTCACACTGCCA
TGAAGTGTGACATGGGGCTAGGAGAGGGTGTTTTTGGTGGCAGTTGGAGGGGGGCAGTGAGGTCTGAAGTCTGGATAGGAGTTCCCCAGGTGTT
CTGCAGGCTAGGACTCAGCAAGTTCACAGGCCCCATGGCAGGATGGAGAATGGCATACAGGTGGGGCTTGTGGAGGGCAGAGGGGCCAGAGCAA
GGGAGCCCCATGCAAAGCAGTCCTCAGCAGTCCGTTCCAATAAAGCGTCAGAGTTTCCAAATGACTGACTTAAAAAAAATAAATCCCAGCCAAT
GGAGAGAATTTGAAGCCACCCCTTGATGATGTCACTAGCACTCATCTTCCTGATCAGCATTCTTTAGTGGGATGTGCAGAGACTGGCCCCTCCA
CACTCGTCTGTGTCCACGGAATACAGCTATGTCTGTGATAGAATCTATTATTAGCCACATTTTCTAGATGAAGAAATGAGAACACAGAGAAGTT
AGATAACCTGCTGTATTAGTCATCTCAGGCTGCCATGACAAAATACCGTAGATGGGGGTGCTTAAACAACAGCCATTTATTTCTCACGGTTTTA
GAGCCTAGGAAGTCCAAGATCAGAATGCCAGCCTATTGGTTCCTGGTGGTGGCACTCCGCCTTGTGGATGGCTGCCTTCTGGATGTGTCCTCAT
GGTGGAGAGGGCCCTGGTGTGTCTTCCTCTCCTTATAAGGGCACCTAACCCTTATGACCTCAATTAATCTTGATCACCTCCTCACAGCCCCATC
TGCAAATACAGATACTCTGGGGCTGAGGGCTTCAACATATGAAGGGGAGTGGGGTCCATAACACCTGCCCAGGATCACACCGCCCATCAGTGGG
ATTTGAACTCAGGCACTGCCTGTCCAGTGCCCAGGCACCTAACCACTGAGGCTCCGCCCCAGGATGTATGGTGGGGGGTACTGCCCATCCCAGT
GAGAGTTCCTCAGGGCAGCAGGTGTTCAGGGATCCTTCACAAATCCACAGGGCCTTGCTCCCACCTGGCAGCTGGACTTCTGAGCTGGGACCTG
AACCCACTTAAGGGTGATCCCAGCGATTGTTCGGTAGAGGACTGGACTGAGGATACTGCATGGTGAAGCCAACTTGAAACACTTGAATCTGGGG
GTTCAGCCAGTTTCCAATTTACTAAGCAGCCATCGGGTGTGGTCTGCATGACCTTCGGCAAGTCGCTTGACATCTTCCAGGCCCCAATTCCTGC
GCCCTCACAGGAGGCAGGGGGCCATCCCACAGGCTTTGAGGTCGGTCGAGCACCCAGCGGGGAGTCCCTGGCATAACGCAGCCAGGAGGCAGTA
GAGTGGCAGGCGCCCCAGGCGGTGCAGGTGCGGCCGCGGCTGAGCGAGCGGGTGTCCGGTGTCCGCACGGGTGGCAGCGCAGGCCCTGTCGTCG
CCGCCACACCAAGGCAGCTCGGAGGCTGGGGACACCCGCCCGGCCTCCGCGGCGCTGTCGCCCAGCCCTGGCACCCCCCGGGCCGAGTGCGCAT
CGGGCCCCGTGGGAGGCCCTGGAATGCGTCCACCGCCTGACCCGGAGCGACCCCCCGCGGCGCCCTCTCCCACCGCCCCGCCCGGGCGCCCGCT
TCCTCCCGCTCCCTGCGCGCCCCCCTCCCGGCCGCCAGCCAACTGCGTGCCCCCAGGGCCGGCAAGAAGCCACATGCCCCCGCAAGGAATGCCG
GGCCTGGCGGGCGGGGGGCGGGCGCTGGGCAGGGCCGGGCGGGCCGGGCCGGGCAGGGAGCTCCCCGCCGGGAAGGCTGGCGAGAGGGGGAGAG
CCCGCCCGCGCCCGCCCCGGCCGCAGCCTACACCGGCCCGAGACGGGGCGGCCACGGGGCAGGGGGCGGCGCGCCCGGCCTGGGCAGCCCTCCC
CTTCCCCACCGCGCGCCCCCGGCCCTGCTGGCTCGGAGGAGGGGGCAGGCCGTCACGTTTCCCCGCACCCCTCCCAGTCGCCGCCGCCGGCTTT
GGGGCCCCGGTGGGGAGCAGGGGGCTGATTAGCCGAGGAGCAGGGGTGTGGCCAGCGGGGCCCCGGGTAGTGAGTCACACACGCCGCTCGCCCA
GTCCCAGGGCCCAGCGCCGTCCCAGCCCCAGGCTGCGTGCCACCCTCGCCCCGAAGAGTGTCCCCGGGCTTGGCATGAGGACCCCGGGGTGCCG
AGGGAGGGGCTGAAGCCCGGCGCGGTGGTGGAGGGCGGTAAGCGGCGGGCTGGGGAGTGTCCCCTTGAGAGAGTCGGGTGGGGCTGGAGCCCTG
GCTGGCCCTCGCCAGCGCCCCCAGGCCCTGCCTGTACCTGCTCTGAGCTGGCAAAGGGGCCTGGCTGCGCTCCCGGCAGTCCCAGGGTGAGGGG
CTGCCCTGCCAGGCGCTGAGGGCCCGTTCGGTCTGCCCACTACCCCTTGGCCCAAGTTGGGCTTGAGTTGTGAACCCTCCCTTCTCGCCCTTTG
CAGTTTTGCGCCCAGGCTCATGTGGGATCCTCCTGCCCTTTGCCCTTTGGTCTAGGGGTCCAGGCTATGCTTCCCAGCGCGGCCTCCTGGCCTG
AGCTGCTCTAGGCTCCTGACCCCATGGGGCTGGTGGCTCCAGTCGTCCCCAGGACCAGCCCGGGATAGAAACGGCATTTCCTCTTGGCCGGGCT
ACAGCTGTTTTTGTTGTCTGTGTTCTCAAGTGCTTCCTCTGGGGATGGCAGGGAGGGTAACCAGACTCCGCGAGACCCCCACTTGCCCTCTCCG
CCGCTCACTCCTCTTCCCCTGTTTCTTTGATCCTCTTCCTGTGCTGGTCCCACCTCCTGCGTCCCAGGACAGAATGACCGAGAACATGAAGGAG
TGCTTGGCCCAGACCAATGCAGCCGTGGGGGATATGGTGACGGTGGTGAAGACGGAGGTCTGCTCACCACTCCGAGACCAGGAGTATGGCCAGC
CCTGGTGAGGCCCCTGTGTGGGAACTGGAGAGGGAGAAGTGGGAGTGGGAGGTGCCTGGGCTCGGTTGGTTGGGAACCCTGGGAAACGTTCTCA
CTCCTTGCCCACCTTCCCTAACCCTGCTCCCGGCAGCTCCTGGGCGCAGGCTCTAGAAAAATCCAAGCAGTGGCCTAGGGAGGTCTGTGCTCGC
ATCTCAATCCTCCAGTTTTTCCACCACCTCCTGCACTGAACAGCAGGCTGGCTGGGACTCCTCTGCCCACCCCACATCTTCTCGGTCTCCTGGC
CAGGTCCCCAGCTTTTGTCCCCACTGGGCTCTCACTCTCTGATGACTTCTTTGCTTTCTTTGCGGGCCTCTGCCCTGGCCAGCTCTAGGAGACC
GGACTCCTCGGCCATGGAAGTTGAGCCCAAGAAACTGAAGGGGAAGCGCGACCTCATCGTGCCCAAAAGCTTCCAGCAAGTGGACTTCTGGTGT
AAGTGGAGCTTGGGGCTCTGGGCTGCTCCTCCCTTCACCCCCATCGCCCCATTCCTGGCTAGGGAATTCACAACAATGCTACTAAGATAGGTCC
TCACCTGCAATATACCAGGCCCAGGTCCAGGCCTTGGTTCGTCATCATCATTAGCCATCCAAATGGCTATTGTTGGCCCCATTTTCCAGATAGC
ACACTGAGTCTTGGTGAGGGTTAAGTATCCCACGCCTCGAGTCCTCTGGCCCAAAGGGGTACAGTTGCACTTGGACTCCAAAGCCATGCTCTTT
CTTCCAGTTTTTTAAAACTTGAGACAAAGGTAGCTGCTGGCCTCCTTGACGTAGGCAGGCCTGTTCTTGGAGCCCCCAGGGAGAGTATGGGTTA
TTGCTACCGATGACCCTGGGGCCTGCAGCTGGCTGTCCATGAGTGGGCCTCCTGTCAGCCCCTCTACCTCCCCCATGGGGGTCTTACCTCCCTG
CAACAACTGAGGCCCTCCCTTCTCTTTCCATCCCTCCAGTCTGTGAGTCCTGCCAGGAGTACTTCGTGGATGAATGCCCAAACCATGGCCCCCC
GGTGTTTGTGTCTGACACACCGGTGCCCGTGGGCATCCCAGACCGGGCGGCGCTCACCATCCCACAGGGCATGGAGGTGGTCAAGGACACTAGT
GGAGAGAGTGACGTGCGATGTGTAAACGAGGTCATCCCCAAGGGCCACATCTTCGGCCCCTATGAGGGGCAGATCTCCACCCAGGACAAATCAG
CTGGCTTCTTCTCCTGGCTGGTGAGTGTGCCCTGGGCTATTCATGGGAGAGGTTGCCAAGAAACATGGAAGGAAACCACCAGGGGGAGCCATAC
TGGCCCAGCTTGGCCCCAGAACTTTTCTACCATCGCTTCTGGACCTGTCGATGATGGATCTTGCTGTCCCCTGGCCCCGGCTGAGTGGCTGCAA
CGCTTGGTGTCCAAGGGCAATGCTGAGAGCACCCACCTGGCAGTAGTCAGCAGATCAGGAAATCACCAAGCAGAACTCAGGGCAATTTCCCCAT
GAATCCTCATGTGTGTGTCTGGTGGACCATTGGGCAGTATTGAACCAACCGATCTTCTAGAGTGGGTGACTCGGAGCTGGCCTTTGGGGATGCA
GAGCATGACTTCCCACTTCCAAACCAGTAATATCAGGCTCTGGGCTAGGTGCTGGGGGTAAACCCTTCAACAAGACAGACATGGTTCCTGCCCT
CAGGGAGCAGACGATCTGGTAGGAAGACAGGCATTGAACAAGCAGGTACACATGTGATATCAGTTGCAACACAGGGTACTATCAATAGATGGGT
TGCAAAGATGAGGAAGAGGGGTTTTCAAGGGTATGAGGAGGAAGGGTTACCAAAGCAACTTTAAGGAATCATGCTTGAGCTACTAGACAGAGAT
AGGGGAGGGTGGGGTAATAGAGAAAAGCATTCTAGAGGGTAAGAAGATCCTTATTCACTACAAAAACACTTCTTGAGTATCTCCTTTGTGCCAG
GCACTGGGCTGGCCCTGGAAATACAGTGGTGGGCCAGATAAGGCAATCAGATAAATCCTGCCTTCATGGAGTTCTCTGTGGGAAAGATAGAATT
AATCAAAGACTAAGACAAATCCATGTCAAATAATAACTCTGAGGGTGATGGAGAAACAGTGAGACTGGGAAAAAGTGAACAACCAGGGGAGGTA
ATATGGGTAGAGGTCAGAGAAGCCTTCCTGCTAGTTGAGCTGTGATTGAAGGAGGTGAATGCCTACCAGGTGAAGATTGGAGAGGTGCATCCAG
ATGCCTTGGACAGCAAGAGCAAAGGCCCTGTGGCAGGAGAGCATGGTACATCTGAGCTACTGGATGAAGGTCAGTGTGCCTGGAGTGCAGCACA
CACAGGGGCCTGGAGATGATGGAGGCTTGATTTTGTAGGGCCCAGTGGACCAAATTAGGACTTCTGTCCTTTTAATAATGTAGGAGTTGGGGGA
GTGACTTGGTCACATTTATGTTTTGAAAAGATGACTTTGGCTACAGCCTGGAGAACTGATTGAGCTCAGGGGTGCTGGTCAAGAATGGGTACCC
ACACCCCTTTCCCCAGGTAGAGCGTGTGGGAGTTCTGTAGTCGGCAATGGTGGAAGACTGGGCTAGGGTTGGGGCAAAGACTTGGAGCCAAACG
AACTGCTTTGTGAACTGTATCAGAGAGGAGAGCTCTGACTTAGACATAGACTAACTGGCCTAAGGAAGGGAGCAGGAGGTATCAAGGACATCTG
CAGTCTCCCAACAGGTTGTTAATGGTGTCTTTCTCTGAGAAATGGAGGAGGCTGGAAGGGGACTGGGTTGAGGGAAAAATCATGATTTGGGTTT
GGGACTTGTTGAGTTTGGAGTAACAACAAGATATCCAAGTGGTGATGTCAAGTAGGCAGTTGGATATATGGGTCCGAAGCTTGGAGAGGAGGCT
TGGAGATGTTGCTTAGTGAGTCACCTGTGCATGGGTGGTCAGTGGGGGTGTGGGTGTGGAGCGCAGAACCTAGGGAGAATGTGCTGAGTGAAAG
GTGGAAAGGCCTGTGAGCAAGCTTTGAGGGACTTTAGCAGTTCATGGTCTAGAAGGGGAGCTTGATCCTGCAAAGGAGACAGAGAAGGTTTGCA
GCCAGAGAGGTAGGAGGGAAACCAGGAGAGTGGGTGACCTGGAAGTCAGGGGAGAAGAGAGGTTCTCCAGGCCACAATGGCTGTCAGTGCCAAG
TGCTGCTGAGAAACCAGCCAGAAAGACTGAAAAGTGTCCCGTGGATTTACTACCAGGGAAGCCATGGGTGACTTTAGCAAGAGCTATTGTAGAA
AGCAGTGGGTCTGAACCTGAGGCAATTTTGCAGCCCAGCCTCCTGTCCCCGGACATTGGGCAATGTCTGGAGACCTTTTGGTTGTCACAGCTCA
GCATAGGGGTAAGCTGCTGGCATCTAGCAGTTGGAGACCAGGATTACTGTAAACATTTTACAATGGACAGAATAGTCCCTCATAACAAAGAGTT
ATCTGCAGAAACCCTGCTGGAGAGTGATGGGGGTGGAACCTGATTAGCATGGAGGTGACCAGGCATAGTCATTTCCTAGAAGTTTGGTTACAGA
TAAGATCAGAAGTGGAGGAGAAAGATAAGAGTGAGGTGGGAGGGTGTGTGGTCAAGAGAGTTTGTTCATTAGAAAGACTTTAGCATCCTCAAAA
AGCCAAGGGAGGGGTTCTGCTGAAGTGCTTGAGTGTAGAGTAGAGATAAGGCATGATTCCTAACCTTTTACATCACCTCAGAGGGTTCACATGG
AGATACTGGACAAAAAAGGGGGTGGATCAGCATCATTTCTGTCCCTACAGGGTTAGCCCAGAGGATTAGAGCAGATAGAGATAGTTAGGTATAG
TAGGCCACATAATGTCCCCCCAAAAATGTCCCTGTCCTAATCCCTAGAACTTGTAAATATACTATCTTCATGGCAGAAGGGATTTTTTGCACAT
GTGATTAAGTTAAGGATCTTGAGATGGGAGCGATGATCTTGGATTACCTGGGTGGGCCCAGTGTAATCACAGGCATCTTAATAAGAGGGAGGTG
GGAGGCTCAGAGTCAGAGAGAAAGGGATTGGAGGATGCTGCTCTGCTGGCCTTCGAGATAGAGTAAGGAGCCGGGAGTCAAGGGATGCAGGCAG
CTTCTGGAAGCCAGAAAAGTCAAAGAAACACATTAATTCCCAGAGCTTCCTGAAAGAATGTGGCCCTGCCAACACCTTGATTGTAGGACTTCTG
ACCTCTAGAACTATAAGGTAATACATGTGTGTTGTTTTAAGCCACTGTGGTTTTGGCAGTTTATTGGCAATTTATTACAGGAGCAACAGGGAAC
TCATATAGGAGGCTTTTATGTTTGTGGGGGGACATCTACTAAAATATGCAGGGAGGTGGATTGAGGTGGCCTGAAATCGAGGAGATCAGAGAAG
GTGAGTTCTGCTTGGGATGAGTAGAGTTCCTGGTGCCCGTGAGATGCTCAGGGGAGCTGTCCAGAGCACTCTGGTGTGTGTGGCTCTGCAGTAG
AGAGATGCTGGGATGACAGGGAAAACTAAGTATATAAATTAATGCAGCTGAAGCCCGGAGAAGGGAGGGCATTTCCTACAGAAAAGGTGCTTGG
GGAGGAGAGAAGAGGCATTTCCTTTGGAATATGAGTTTGAGGAATCCTTGGAGGGAGAGGAGTTGAGGAGGCATGGAGCAGTAGGAGGAGACAG
TAGGAAGTCTCCTTGGCCCCCTTGGCAGGAGCAGTCTCATGGGCGGAAGCCAGATTGCCATGGATTAAGGTGTCAGTGGGAGGTGAGGAAAGGG
AACTAGCAAGGGTGCATGGCTTGTTTTAAAAGTCTGGTTGCCCACTGAGCAATTATTATATGCCAGGTGCATACTGGACACCTCTGGCATCTCA
TACTCTTCCTCGAAACAGTCCATAGGGTGGGGGTTGCTGCCCCTCTTGTTCTGACGAGGAAACACAGGACTCCCACAGCTGGTCCACAGTAGAC
CTTGGGTTCCTTCAAAGATAGTGTCCCTCCACCATGTGAGGACTTCCCTTATTGGGAGACATTGCTTATGGGGTGGAGCAGTTAATAGCTACCC
TGAGCCAGTTTTGGTTATGTGTCCAGGTTCAACCCAACCTCACATACACAGAGCAACTCCAGAGCACTAGGTCCTGAGCAGGGAGCTCTGGGGA
TAACAGGGCTGCGTGAGGTTTGCTGTCTGTTCTTATGGAGCCTGTGGTTCAGTTATGAGGAGCAGGGGCCATGGTGGATAGGAGGGGAGGAAGC
TTATTCCCAACCAAGTAATACAGAGGCAGAGTATTGCTTGGTGATCGCAGTGCTGCAGAGGGCTGTAGCAGTAGGGAGAGGGGGATTGGTTTCA
ACAGGGCAGATTGGAGATGGATCTTGTCTTGAAGGATGGATAGACGTTCAACAGAGGAAGCCTATGGCTTCACAGCCTATTGGAAAATTGCAGG
AGAAAAAATCTAGGACTGTGCCCAAGGAATGGCTATTAGGATCCCTTTCTGCAGCTGCTAGAGTCTGTACCTTGGGACAGCCCAGCCATGGAAA
GTTGAGAATCTTTAAAGCAGATGAACTTTCCCAGGTTGGCTGCCTGGCACAGGTCTACGGTGAGAAGAAAGGAGGAATAGAAGGCCCTGGTGTT
TGCAACCTGGTTCTAGACTAGCTCCACCAGTCACGAGTAGGCGTGGCCCCAGTTCAGGAGGTGGGACTGGTGGCCTGGAGGTTATTCCCTCTGA
GGTTTCTTCCTAGGGCCTTAGCTGCAGCCAGCAGCCAGCAGCCAGCAGCCAGCAGATGGGCACAGGGAGGCAGTGGTTAGAGGCTGCAAGGCCA
TATGGGGGCTTTGACCTTTGGGAACCCAAGAGGTGAGAGACATTGAAGGCCTAGAGCCACCAGATGCTTAAAGTGGCTCAAGAAAAGTTCGTGT
GTGTGTGTGTGTGTGTGTGTGTGTGTGTATGTGTGTGTTGGGCATGGGTTTTTGGCCTCCACTGGGGAAAAGCAGGCGTGAAGAACGGCTCTGC
CTGCTCTGCTGATGGGTAGGCCAAATGGTGGGAGAGTACGGGGAAGGTGGGGTGAACAGTGGTGCTGATTTTTGGACCAAGATCTAGAAGCCCC
ATATCCTAAATAAACTGCCCTCATTTGTCAAGTCCTGTCCCAAAGCCATACTGGATATCATCTTCTTTGGACCTTCTGCCTGCCTGCAAAAGCT
CTGGCCCCCATTCAGCACAACACATTTCCCTCCACACTCCCTCCCTCAGTATATGCACTCTACTCTGTAGAAATGATGGTGGTGGTCATGACAA
GCATAGCTGTTAATGTTTATTGAGCACTTACTATGTGTCTGGGTCTGTGCTCAGTGCTTTATAGCATAATCTCATCTGATCCTAACAACGCTCC
TGTGACATAGGTATTATTCCATCCATTGCACAGCTGAGAAAACTGAGGCCTGGAGAGGATGAGTCATTCTGCACAAAGCTAAACAGCTGGTGAG
TCACTGAGCTGGGGTTCAAACCTGTGCAGTCTGACTCCAGAACCTACCCTCTCATTCCGCCTCTTGGCCCTCTAGCCTCCCTTACTTAGCACGC
CTCTTCTTACCTCCTTGCCTGGGTAGACTCTATTCTACTCATTCTTCCTGCCCAGCACAAGTTCCCTCTTTGTCTTCGCTTAACCTGGAGGAAA
GCTGGCCTGACTTTGGGCAGAGTGGTACAAGGCATGGGCCTGGGTCATAATGGACCGAATGGCCATTTCCACTCCACAACTCTCCTTCGTGGCC
TGGCTGACTTGGCAGGAGTTGGACACTCACCTCTGCCTGTCTAGGAAAGACATGCCCCTTGCTCTGCTTTTTCATCCTATTGGAAATTATAAAT
GCTGTGATTCATTGCCAAGATCACCCAGGCTACCCTCAGACACTGCAGGCCAGGAACCATGCTGCTGGCACGAGGCATTTGGGGGTGCTGCGCA
GCCATGTGGTTTGGTAAAGAGGCAGAAGACAGAGGAAGCCAACATGGGTTTTTTTGTTTGTTTGTTTTTTTGTTTGTTTGAGATGGAGTTTTGC
TCTCGTTACCCAGGCTGGAGTGCAGTGGTACGATCTCAGCTCACCGCAACCTCTGCCTCCTGGGTTCAAGCAATTCTCCTGCCTCAGCCTCCCT
AGTAGCTGGGATTACAGGCATGCGCCACCACATCCAGCTAATTTTGTATTTTTAGTAGAGACGGGGTTTCTCCATGTTGGCCAGGCTGGTCTTG
AACTCCCGACCTCAAGTGATCCGCCCACCTCAGCCTCCCAAAGTGCTGGGATTACAGGCTTGAGCCACCACACCCGGCCGCGAGCACAGGGTTT
CTATGTAAGTTTTTCGTTCAGACCTTGGCTTGTTCCAGAAGGGATTTAGGGTAGCCTAGCAGTATACATAAAATATACCACAATGGCATATTAA
AACTGGGAGAGAGAAAGAAGAAAGGAAAACAAGGGTGGAGACCCTAAGTGAAGCCAGGAATGAAGCTAAAAATGCATACTATAATGACCTCTAT
CTGCACTTTTAGCTATACAGGCTCCAAATCTGGCAGCCACTGGGAGAGTAGCTGGTACATTCTGTCCATAATGTGAAAAGGACCAGGGAGCTCC
TGAGGAGGTCCGCCCTTCTGGGCGCTGATACTGGATAGGTTTCCCAGGGGCCCTTATAAAGAGGACACTGTGTGGTGGAATTGTCCACTCCATG
CGAGGCAGCCTCCCCACCTGCCAGACTGCTACCGTTCTGGGGAGAAGCAGATATGAAATGGTGTTTTAGTGTTTCCCATTTCAGCTTTTATAGT
AAATTGACAATTTCTTTTAAAACTTGAAAGTGGACGTGCCAGAGCTCATGGCACATCCTCCAGAAAAGCCTCTGAGAGACCCTGAGTGCTTAGC
TAGATGAAATAAGCAGACAGTTGCAGTGCCATGACATGTGGTGGGGACAGGCAGGTGTTACCAAGTTCGGGGATGCTCAGAGAAGTGAGGAGTA
ACAGGTTTGTTCGAGAAGAAAGAAAAGGGGCGTTTGAGACAGGGTAGTCGTGCAGTGTGTGAGCATGCACGAGCAGGTGCTTTTCATGCTTGCT
TGTTAATTTCTACTCTTTATTTTGAAAGTTGCAAACTCGGTTGAAAAGTTGCAAGAATAGTGTAATGAATACCCTCACACCTGCCTGTAGATTC
ATCTATTGCTAACACTTTATACCCATTGCTTTATCTCACTTTCTACACTTACACCACCTACACACACACGTGCACACACACACACACTTTTTTT
TTTTTTCTTGAGATGGAGTCTCACTCTATCACCCAGGCTGGAGTGCAGTGGCATGATCTCAGCTCACTGCAACCTCCACCTCACAGGTTCAAGC
AATTTTACCACCCCAGCCTCCCAAGTAGCTGGGACTACAGGCGCACACCACCATGTCTGGTTAATTTTTTTGTATTTTAGTAGAGATGGGGTTT
TACTGTGTTACCCAGGCTGGTCGCAAACTCCTGAGCTCAGGCAATCTGCCCGCCTCGGCTTCCCAAAGTGCTGGGATTACAGGCATGAGCCACC
GCGCCCGGCCATACATTCTTTTTTTTTTTTTTTCTGAACTGTTTGAGATATAGTTACAGAGACATCATGACACTTTACCTCAAAACACTTCAGC
CAAATTCCTAAGAGCAAGGTGTTCTTCTATACCAGGGTGAGCAAACTTTTTTTTTTTTAAAGGCTACCTGGTAAATACTTTCGGCTTTATGGAC
TGTGCAGCCTCTGTCATAACTACTCCACTCTGCCTTTGCACTGCAAAAGCAGTCATAGATGATACATGAATGAATGAGTGTTGCTATGTTTCAA
TAAAACTTTATATGAACACTGAAATTTGAATTGCATATTATTTCAAATGAAATTTGAAAGTCATAGAAATAAAATTACATAAAAATAATTTTTT
AGTCATTAAAAAAATGCAACATGATTCTTAGCTTGCAGGCTGTATAAAAACAGACAATGGGGGCTGGATTTGACCTACAGCCATAGTTGGCTGA
CCCCTGAGCTATTTAACCACCATATAATGATTACACCCAGGAAATTTGACATGGATACAATACTGTCTCAGCTATAGTTCGTATTCAGATTTCC
CTAATTGCCATTAGTAACATCCTTTGTGGTTTTTTCCCCATCCAGGACCCGTGCAGGTATCACACATTGCATTTAGTTGTCACATCTCATTAGT
CTCCTTTATTCTAGAAAAGTCTCCCCTGTATTGTTTTTTTTCTTCATGATATTGATACTTTTGAAAAGTTCAGGCTAGTTGTTTGCATAATGTT
CCACAGTTTAGATTTGTCTGATTGTTTCTTCATGGTGGATTCCGATTACAGCTTTTTGGCACAAGCAATGTTAGGTCTTTCTCACGGTGTGCCA
TCAGGAGGCACCTGGTGGCAGTTTGTCTTGTCTTTTACTCAATGGTTTTAACAACTATTGATGATTCTTGTCTGTATCAATTATTATTCATGGT
TAAAATGATTTTTCTATTTTTTCTCTTCTACATTTATTATTTGACATTCTTCTGTTTAAAAAAGATAGTGCTCAACCTTCACCCCCACTCAATC
CTCATTTAAAGCTATTTTTAGTATTCATATGAACATGAATTCTTTTTTTATTCAATGTGTTGGAATCCATGTCTGTCATTATTCATTTTGATGT
TTAAATTGCCCTAGATTTGGCCAGTGGGAACGCTTTCAAACTGGCTCCTTGTTCTTTTGACACATCCCCATCATTATTTTAGCACTTTTTCCTT
ACTTTCTGGCACAAAAAGATATTCCCACTGTCTCTCGTAGGAGCTGTGATTCCTTTCAGTGGGAAATGGTGTTTAGGAGCCAAAATCTCTGAAT
GGTAGGTGTGCAATTGCTACTGGGGTGTCCTTGCTTCTAAGCCAAACTAGGTTTTATTATTATTATTATTATTATTACTATTATTATTATTTTA
TTTTACTTTGAGTTCTGGGATACATGTGCAGAACCTGCAGGTTTATTACATTGGTATACATGTGCCATGGTGGTTTGCTGTACCTAACAACCCG
TCATCTAGGTTTTAAGCCTCCCATACATTAGGTATTTGTCTTAATGCTCTCCCTCCCCTCACCTCCCACCCCCGCAACAGGCCGCCGTGTGTGA
CTTTCCCCTCCCTGTGTCCATGTGTTCTCATTAGCCAAACTAGGTTTTGAGGGAGGGATGGATGAGTTGATGCAAATGTCCTACCATGTCAGAA
ACTGAGGGAACGCAGCATAGCAGTGGTCATGGAATTTACAAAGGCCCTGGGGCACACACGTAACAGTCAGGTGGGGATGGTATTAATATTAGGG
TATAACAGAGGTACCAAGTGTGTTTAGAAGGAAAAGTGGAAGATGAGACTAGAGTGAAAGTCACTTTTCTTGTGTTCTCAGATGCAATGGATCA
TAAAACACTGTTGATTCAATATCAGCTTTTCCAGGAGTAAAAATGAATACCTCAATCCAAAGATTCTGAATATCAGAAGCATTAAATGGGCTCA
GCTTGTGTAGCTGTTTTATAAGTGCTGGTAGTTTAGTTATTGAATCACAAACCAAACCAAAGATTTCCCCAGGAAAATGTAATATGCTCATGAA
CCAGTTCTGGCTGGAGCCAAGTCTGTACATCTATTGTATCAGAGCAAAAGTAGAACATGAAAAATTATTCTGAACCTGTATAATATTTCCAGTA
CAGACCAGCTCCTAACCTGACCTCCTCTATTTCTTGGCTTTCCAGTCCATTCTACTGTAGCTGTGTTTCTCACACCTGAACAGTGTTAAGTTCA
GGTGATTTTTAATGTTCCTGGAGTCTTCATGTTACATTAATTTTATTTTCATGTTACTTAAAAAGGTATAAACATATAGCTTGGTAAACACAAA
GTGTCTTGTACTTATAACTTTTAAACAACTGTAAAGCCAAAACAAGTTTACACATATAAAATTAACTACAAAAACCTCAACATAATACACATTA
ACAAAGTCATTCTAATGGGATAGTTGATGATGTGTCACTGAAACAAAATTGCTATTTATAAGGCGAATATCGTAGTGACTACTGAGACACAGCA
AGAGTCTTTGGAGTTTGGAATGCCTGTACTTTTGTGTTCAGTGTGATATCTTTACTTTCCTACTATTTGTCTCTATTTGAAACACTGGGAAATC
TTTTATTTATGTATGTATTTATTTATTTAGAAATGGGAACTTACTCTATTGCCCAGGCTGAGTGCAATGGCAAGATCATAGCTCACTATAACCT
CAAACTCCTGGACTTAAGCCATCCTCCTGCCTCAGCCTCCAGAGTAGCTGCGATCACAGGAGTGTGCCATCACACCCATCTATCTTTGTTATTG
CAACTGCCATGATGTGTCTTGGCCTTTGCTTTGCCGGAGTGCAACATTTACATATTAAGTTTATATCTGTTCTTTTTTTGTTGGCTCATAGCAA
GTAGTCATAATTGGGGTTGTATGGTCAACTGTTGGGGTAAAACAAGCTACCTAAAAACCCAGTGGCATAGAACAAGGGTCAGCAAGTGTTCTCT
GTAAAGGGCTAGATAGTGAATATTTTAGGTTTTGCACACTATACAGTGTCTGTCTCAATGACTCAATTCTGCCATCATGGTGCAAAAGCAGCCA
TCAGTGTGCCATGTTCCAATAAAAGTTTATTTATATATAGTGAAGTTTGAATTCCATATAATTTTCACATCATGAAATATTGTTTTTCTCTTGA
TTTATTTTCAACAATTTAAAAATCATTCTTAGCCTGGTGGGCCATGCAGAAACAGGTGGTGGGCTGGAATTAGCCCACAAGCCGTAGTTTGCTG
AGCCCTGACATAGAACGACAATCATTTACTCTTGCTCACGTATCTGCTGGTCAGCTGCCGTTCCTCATTTCTACTTGTGTATCTGCTGGTTGGC
TGCAGTTTGGCTGACATAGCTGGGCTTGCTGGGCTGCCCGGACTCCAGCTGCTGGGGGTTCTTGGCTTCTCTCTCTGGAGCCAGGTCTGCTCCA
GACACTTGGAGCATGTCTGTTCTGGGCCCCAGCTGAAGGAGCAAGGGCTCCCTGGGGGAGCTTTTCTACATTTTGAGGCTTTGCTCATATCATG
TTTGCTAATATCTCATTGGCTACATCAATTTCACAGTTGAACGTAAGGTCAAGGAGCAAAGAAGTGCAGGCTGCCTGTTAAACAGTCTAGTTCA
GAATCCTGTGGCAAAAGGTGGTACGGATGGCTGAAGAGCTGGGGCCAGGGATTCAATCTGACACGGATGCCACCTCAGCCATAAATTCAGTACT
CTTGCGGGTGCAGAAATCTCATGAGGATTCCCGCATATTGATTTTTTTAAATGAAAACATGGAATTAAAAATTCTTAGAGAAAACATTCAGATC
CTGAGAATATATCCAAAGACCCTAGTTTGAGAGACACTAATTTTTAGTAAACTTCCTGGCTTTGCCGCAGAAGATTTGGGGTTTCTTGGTGTTT
GAAAATTCCCCAAGGAGAGCTCTTGTTGAACTAGGCTCTCTGAACCTAATTCAGCACTCCAAACCCCAGTGGGTCCTCCAAAGATGCTAGTTAG
GGTTGATGAACAACAATAACAACAATAATAGTAGTAACAATAATAGCTGCTGTTCACTGAGTACTAAATGATTTGTATATCTCACAACAGTACT
GTGGGATAGTTATTATCTTCTTTTTTTTTTTTTTTTTTTTTTTTTGAGACGGAGTCTCGCTGTGTCGCCCAGGCTGGAGTGCAGTGGCGGGATC
TCGGCTCACTGCAAGCTCCGCCTCCCGGGTTCACGCCATTCTCCTGCCTCAGCCTCCCAAGTAGCTGGGACTACAGGCGCCCGCCACTACGCCC
GGCTAATTTTTTGTATTTTTAGTAGAGACGGGGTTTCACCGTTTTAGCCGGGATGGTCTCGATCTCCTGACCTCGTGATCCGCCCGCCTCGGCC
TCCCAAAGTGCTGGGATTACAGGCGTGAGCCACCGCGCCCGGCCAGTTATTATCTTCTTTTTAAAGATTGGGAAACTGGGGCTTAAAAGGCCAT
GTTACTTATCTAAGGTTATACAGGTCATTTATGGGGCCAGGACTTTTAACTAGATCTCCAGTTCATCATCTCCAGGCCTCAGATGTGAATATCT
AGAATGCCTTCCTGGCATCCCATTGCTGGTCTTCCCACTTGCATGGAAACCTGTTTCTTTAGGGGGTCACTGTTCAAGCATAGGATATGTAGGA
CGTAAGATCGTTCCCTTCTCCTTGTTTGATTGGTAACAATGAACCTTTGGCAGATTTTATTTTTTAACAGCTTTATTGAGATGTAATTCACATA
CTATATAATTCACCCATTTAAAATGTATGATTCAATGATTTTGACTATATTCACAGGTATGTGCAACCATCATCACAGTCGGTTTCATCAAATC
CTGTATCCTCTGGCTGTCGTTCCCCTCTTCCAATCCCCTTAACCCACATCTTCAACCCCTCACCCCCACCCATCCCTAGGCAACCACTAATCTA
CTTTCTGACCCTATGGATTTTTCTATTCTGGCCTTTCATATAAATGAGATTATGTAGTATGTGGTCTTTGTGACAAGCTTCTTTCACTTCGCTT
CATGTTTTCAAGGTTCATCTGTGTGGCAGCATGTATCATTCCCTTTTACTTTCTTTCTTTTTTTTTTTCTTTTTTTATCTGAGACATCATCTCT
CTCTGTCACCCAGGCTGGAATGCAGTAGCTCGATCTTGGCTCACTGCAGCCTCTGTCTCCCGGGTTCAAGTGATCCTCCTGCCTCAGCCTCCCA
AGTAGCTGGGATTACAGGTGTACACCACTATGCCCAGCTCTTTTTTTTTTGTACTTTAGTAGAGATGAGGTTTTGCATGTTGGCCAGGCTCGTC
TTGAACTCCTAGCCTCAAGTGATCCACCCACCTTGGCCTCCCAAAGTGCTGGGGTTACAGGCATGAGACACCACACCTGGCCGTATCATTCCCT
TTTATGGCCTAAAAATGTTCCATATATTGTTGATCTCTTCATCCATTGATGGACAGTCTCTGCCTTTTGGCTATTATGAATAATGCTGCTGTAA
ACATTTGTGTACAAGTTTCCATGCGGACACGTGTTTTTATTTCTTTCGGGCATAATACCTAGGAGTGGAATGGTAACTCAAGGTTTCATCATTT
GAAGATCTGCCAGGCTGTTTTCCAAAGCAGCTGATCAATTTTGCTTTCCCACCAGTTGTATATGAGGGCTCTGATTTCTCCACATTCTTGTCAA
CCCTTGTTTTTCTCTGACTTTTTTATTCTAGCCATCCTAGTGGATGTGAAGTGATACCTCATTGTGAAGCATGAGGGTTTTGCTCTGCCTTTTT
TCTATCTCTTGTTCATTTCAGTGGTTATTTGGGGAGAGCTACCCAGGATGGATGCGCCGGTTGTGTACTTAACAGCTCCATGGGGTGCTTTTCA
TGTCAGCCCTGTTAACTACGTTCTTCTGCCAGGTTAACTTGGAAGACTCTTCCAATCTGCAGCATGATAATTAAAGTGTATTTCTATCTGATTT
CTCTCTGGCTCATTCCAGTTTACATTTTTGCTGCCCCCTAAATAGGCAGCAAACTCCTGATCCAGATACTCCTGGATCTCTCTCTCACCCTTGC
CCAAGACCATTCATAAAAAGGCATAACTAAGAGATGGGATTGAGGCCAGTCTGCTTGACAGCTGCTTCTCTAGTTCTTCCCCAAGGGTCCAAAC
TAGTACTCAGAATGCTATTGCCTTTCACCAGGAGCCTGCCTCCTTACCAAGGAGAGCAAGGCTGTGTGTGGTCAGGGTAGCGGGTGGTTGGTCC
TGAGGCTGGTCCATAGTCCTGGCACCTTTTACAGGCAGAAAAGAAGGACTTGTGAAGGGAAGAGCTGTCTGAGTTGGTTATAGGCTCTGTTCCT
GGATTCCGATCCTGGCTCTACCACCTGCGAGAAATGTGTCTTTGGGCTGCTCACCTATTCTCTCGGAGCTCCAGTGGTTTTATCCATAAAATGA
GGATGCACAGCCTAATGGAACACACCCCTATGAGTTGTTGTAAATACTAATGCTTGTTGCATGCCTAGTACTTAGCATGTGATGAGCAGATCAC
AGCCAGCTTTAGTATCCACAGTTATCATCAGATGGGTTTCAGGAATGGTTGAGGGTGGGAGGTGAAATCAAAGTGTATAAAACCTGGATGTGGA
ATTTAGGATTGTTTGTCAACATGCCTCATTAACTAGGTCTCCAGAGTCTTTTTAAGCAGTAAAAAGAAGGAAATTCTGCCATTTGCAACAACAT
GGGTAAACCTGGAGTACATTATGCTAAGTGAAGTAAGCCAGACACGGGACAAATACTACCTGATACCAGTTATAGGAGGAATCTGAAATAGTCA
AATTCATAGAGACAGAGAGTAGAATGGTGGTTTCCAGTGGCTGGGAAAGAGGGAAATAGGGAAATATTAGTCCAAGGGTATAAAGTTTCAGTTA
TGCAAGATGAGTAAGTCCCAGAGATCTACTGTACAGCATAGAGCCTATAGTTTACTGTATTGCATGCTTAAAAATTTGCTAAAAGGGTAGATCT
TATGTTAAGTATTATCACAATAATAATAATAATAAGAGCAGGAGGAATCTTTTGGAGGTAATGGATATGTTTATATCATAGTTTGTGGCGACGG
TTTGACAGGTGTACACTTATTTCCAAACTCATCAAGTTAGATATGTTAAATACATACAGCTTTTTGTATGCCAGTCATACCTCAAAAAGCGGTC
TAAACCGAAAAGAAATTTGAAAAAAGGACGAGTTCATGTCCTTTGTAGGGACATGGATGAAGTTGGAAACCATCATTCTGAGCAAACTATCGCA
AGGACAGAAAACCAAACACTGCATGTTCTCACTCATAGGTGGGAATTGAACAATGAGAACACTTGGACACGGGGTGGGGAACATCACACACCAG
GGCCTGTCGTGGGGTGCGGGGAAGGGGGAGGGATAGCATTAGGAGATATACCTAATGTAAATGACGAGTTAACGGGTGCAGCACACCAACATGG
CACATGTATACATATGTAACAAACCTGCATGTTGTGCACATGTACCCTAGAACTTAAAGTATAATAATAATAATAAAAGGAAGTTTTGGATTAA
TAAAAGGAAGTTCTCATTTCCTTATCAGTTAAATTAGGGCAGATGGGTTAAATCATGGTTCTTGAGCTTTTTTGAATCACCTAGGAAGTTATAC
TCACGTGCCAATGTTCACAAAATGTTGCACACATTTTAAGGGATGCCCCCTTTCCCAGACACCAGGTTAGAAACCTAGAGCATGGATAATCTCA
GATGCCCTCATCTAGCTTAAGTATTCTTCACTCCATGTCTGTGGCCATAGCAGAGGCCAGAGTTCAAGTTCAAGTTCAGGTTATCTCTATCTCT
CAGGTCTTCAGTTGGCTGGGCTGGGGAGTAGCTAGAGAAAAGAAGTGATGCCATCTGCCTCCTGGGGCTTGGGAGAGGAGCATTTGAGGTATGC
CAAAGTGCTTCATCAACTGTGAATGGCTTCACCAGTCATTCTTGTAACTACTTTGCTCTATGAAGAAATGCAGGATTGAATTATGAATGCTTTA
TGGATTCAATGAATTCCTGGAAAGTTGCAAACAAATCAGGTTTTTGAAAATCCATCTCTGCAGCATCTGGTCATGACACCATTTAGAGGAGCCC
AACAGTGAATCTTCGACTGGGGCAGACAGACATTCTGTTGCAAAGCAGAGCATTCTTCCCTATTAAAATGCCAACCTGCAGATAGCAGATTTGC
TCAGAGGCAAAGCTGGAAGTTCAGAGAGGGGCCCAGAAGAGTCCTGGTTGATTCTGGGTTTCAGGAATGGTTGAGGGTGGTTAATGGAGTCATT
AGGAAAATCTTGGGGCTAACATCCATTGCTCTACAAAGTGGTTAAAGCATGGACTGTACTGTCAGAGACCCAAGTTTAAACTCCAGCTTCCCGT
GTGGAAAATTCCTTTGTGGCCTCAGGCAGGTTACCAACAGCCCTCAGCCTCAGTTTTCCAATCTGTAAAAGGAGGATAATCATAGTAGCTGTGA
GAATTAATAGTGTGTCAAAGCACTTGACATAGTGAAGGTACTCCATAAGTGGTAGCTGTTAATGAGGCTTTATTTTTATTGTCTTTATTAATTA
TTACCATGGTCTAAAATAGCATGTGGATCCACCTGGACCATGCTTCGCTCAGGGGGCCAAACTCCATGCTTTTATGTTATTTTTATGAGCATCT
CTCAGTCTTCATTTTAGACAGAACTTTGAGGTCATTTAGGACAGATTTCCACTGAGTGAGGAAGTCCTTTTTTCAGCCTCCCTGCTAGCAAATC
ATCCCCTGGTTGAACACTCCCATTAACAGGAAGCTCACCACCTCACAGGGTTCTTGTCCTATCTTAGGACTCTTCTGCATGTTAGACCATCACT
TTTTGTGCTGAGTTCAGGCCTGTATCCCTCTAGATGCATGCGCGTTCTAACCATCCTGTCCCCACGACATGGTCGTCCTTCCCACATCTGATGA
TGATGGCTCTGCTTTCTCAGGCCTCTTCTTCTCCAGGCTAAGGGAGCCTCTCACCATGCAGGTTCCCCAGGTTATCAGCCTGGTTCCGGGTTAT
CAAAGGCCTTGTACCACAATGCTCAGAGCTTCTGGGCTGGTCCAAAGTGCTGAGTTCAGAGGGACCAAGGAGTTGACCTTGAGGGATAAGGCAA
GGGAAGTCACTGGGAGACCAGTCTTCTGGGGGGCTTGTCCTGAGCATCTGGCCCTGCTGTCATTCCTGGACCCATGTCAGTCCCAGAATAGGAA
GCCCACAGCCTGCTCTCTACTCAGGATGGCCTGGGCTCATCAGTGGCATGTAACGTGGTCCACAGGCCCTGCGAGATCCTGCCTGCCCTGCACC
CTCTGCCTAGGGCACCATCTCTCTCTTCTCTAAGATCTCAGCTTAGACTCCATGTCCCCAGAAAAGGTCCTCAGTAGATTCCCAGATCCAGGGC
TGCAGCTCAGAGATCCCATAATCCCACATAATTCTCCACCATGGGACTGGCCATGCCATATCACAGTGGCCTCTCTTCTGCGTCCCGCTAGGCT
GTGAGCTTTCTAAGAGCAAAGACACTCTCTGCCTGGGTCAACATTTTATCCCCAGAGCCTGTCACTGTGGCCTCTCCCTCCATGACATTTGTTG
GTTGAATGAATGAATGAGTGAATAAATGCTTGATATGAGACAATCCCAATTGGTGGGAAAGATAAGCCAAAAACACCTGTCAGAAAAGCTCTAG
ACCTGAACCCAGCAGACAGTGGTCTCAGGCGGTTCCTATGTCCTTCACAGCCCTCACCCAGTCCTCTCTGTCCAGATGAAACTGCCAAGACTGA
TTACAACTCAGTGTTGAACCACAGGCCTCCCACCACCCCAAATTAGTAATTAATATTTGAACTCTTAATAATCCCGATTATGATCACAGTGCTA
ACAGTGACTGTTCACTGAGTGTCTTCCACTTCCATTATGACAGCAGTGCTACAAGGTTGGTTTTTATCCAATTTTATAGATAAGGAAACTGAGG
CATGGTCAGCAAAGATCTGCCCAAGGCCACTCAGCTTGTGTGCAGTGAAGCTGAAATTCTAGCCAATTCCTCTGGCTCCATGGCTCTTACTACC
CTCCATGTGCTCCTACTACTGGCTGTAGGAACTAGAGATGCTTCTCTTTTCCCCCAGGAGACAGGGGAGCAATCAGAAATGATAATGTAGGGGG
AGTAGTGTTATGATAAAATCCAGGGTTCTCTGAGATTCCAGGGGAGGGTCCCTGCAGACTGGAGCATCACTTGGGGAAACTGCCATGGAAGGGG
AAGCTTGAGTTGCATGTTTTCTCTTTTTAGTTTTCTGAGATTTTTAAAACACAAAGGCAGTACAGTCTTATAGACAAATTAAGCAAAAGCATCC
CTGCTTCCCTCTTTCCTCCCCTCCAGAGTAATGAGAGTTGAGATTTTGATGTGTATCTCCTAGACTATCTATGCTCATGTAAACATGCATAGTG
GTGTGCTGGTAGATGTTTACAACTGATGGTGATGGTGATGGTGATGGTGAGGCAATGTGCCAATTTCCATGGTGTAAATGCTCCCCCCATGGCT
GATTTCAAGCTACTAGCATGAAATCACTGAATGTGCAATTGGGAAGAAATATGCAATCGCAACTATTGTATGGTATTTCTACTCTGCAGCTACA
GTAGAGGCCAGTACCCTAGAGAGCATAGAAAATCATCAGTCAAATGTAGTAAAATAATTGGGAATGGTTAAGTTTTGAGTATTTATTGCCTTTT
AAAAATATATCATTTATTGAATTGTAAGTTTATATCATTTAGTTTTTCAATAATGGCTGTGTTTGACAACTGGTTCACAAAATTCCTGAAAATT
TAACAGTTGGCTCTCAGTCAACATAAGCTGGCTCCAGCATAGCACTCATTTCATTTTTACTAAAATCTTAACATTTTATAATTTTTTCTTCTTT
CTTCCTTCCTGCCTTCCTTCCTTTCTTGTTTCTTTCCTTCCTTCTTTCCTTCTTTCTTTCTTCTTTTCTTTTCTTTTAGAAACAGGATCTTGCT
TTGTCACCCGGGCTTGAGTACAGTGGCACAATCATGGGTTACTGCAGCCTCAAACTCTTGGGCTTAAGCAATCCTCCTGCCTCAGCCTCCTGAG
TATCTGGGACCACAGGCGTGCACTACCACACTTGGCTAATTTTTTAAAAAATTCTTTGTAGAGAGGCTGGGCGCAGTGGCTCATGCCTGTAATC
CCAGCACTTTGGGAGGCCGAGGTGGGCGGATCACGAGGTCAGGAGATCAAGACCATCCTGGCTAACACGGTGAAACGCTGTCTCTACTAAAAAA
AAACACAAAAAAAATTAGTCAGGCATGGTGGCAGGCACCCATAGTCCCAGCTACTTGGGAGGCTGAGTCAGGAGAATGGCGTGAACCTGGGAGG
CGGAGCTTGCAGTGAGCCGAGGTGCACCACTGCACTCCAGCCTGGGTGATGGAGCGAGACTCTGTCTCAAAAAAAAAAAATTCTTTGTAGAGAC
AGGGTCTCGCTATGTTGCCCAGGCTTGTCTCAAACTCCTGGCCTCAAGCAATTCTCTGGCCTCAGCCTCCCAAAGTGATGGGAGTACAGGTGTG
AGCATCACACTTAACCAATTTTTTTCTCTTTAACTTGCTTTTTACTTAAAAGGCTCTTTTTCCCATCATTCTTTCATTCAACCAGTATTCCACC
ATGTGCCATGCCCTCCCTGCGTTAGGAACCAGGGCCACCATTGTGATCAGGGCAGCCACGTGCTTTGCACTCATTAGCTTACGGTCTAGCCAGG
GAGGCAGTCAGTTACCAAATCGACAAATAGATATCAACAAAATGCTCACTGCAATTAGGGCTCTTAAGGGAAGTTACAGGATGATGTAACAGAG
ATAATGCCATGATAGACCAGGGATGAGGTATGGCCAGGAAGAACTTCCTTTAGGGTCAAGGCTAGGAGAGGCTTCTTCTGTCAAACTCACATAA
TCTGAGACCCAAAAGCTAAGAAGGGACCACATATTCAAAGCTTAGTGGGAAAAAATTGAAAAAGGATATGTTCACTTGGGTGCCATTTCTGTAA
ACTTTACACACATGCAAACAGTCCTGTATGTTGCTCTTTGAAAAGTCCTTGTGTCATAAAAATGGGAAGGCTTCCCACCATCTCTAGGATGGTG
GTTACTCTTGGAGGGAGGAAAGGAAAGAGAAGAAGTTGGAGGCAGAGAGGGTCTTCCACTGCATCTCAGCATTTTCTTGAAGCAAAAGGAGAGA
GAGGCCTGAGACAAATATAACTCGTGTTAATTTTTCTTGGCTTTAGCTGATGGGCACAGGGTTGTCAGCTGCATTCTTCTTTATAATTTCTGAA
TGCTTGAAATATTTCATGATTATTGTTATTTTTAAGGGTAGATAAAAGAGGCTCTAGGCTCACCCTCAGGGCATCAAGGCTGCAGAAAAGTGAA
AAGGAAGTGAGAGGCCCAGGGTAAGTTCGGGGGCAGGCCTGTAGGGTATGAAGGCTGTGGCCATAAATATGAGTTTTATTGTAAGTGCAGTGGA
TGCCATTTTGAGCTGGTGCGGGACATGATCTGATTTACATTGATAAAGGGCTTCTCTGCTGCTTTGAAGAGAACGATGGAGGCTTATGAATGGC
ACTAGGTTAGGTAGGAGTCTAGGCATGGTGACTATCCATCTGTTTCCCATCTCCCAGGAGTTCTCCCAGGCTCTGCTTCACCAAGTCTTCAGGG
GACCAACCCTGATCTAGCTCATGTGTCAAAACTTATCAAGTTGTACCTTATATGTCAATGATGCCTCAATAAAGCTGTTAGAAAGGAAAAAGAA
GGAACATGTCACTATGAAACAGAAAGAGGCAGGATGTATAAAAGGGTGGACCTGAAAAAAAAACTAATTAAAAATTCTGGGAATGAAAAATATT
CATCAAAATAAAGGTACTCATTAAAAAAAAAAACAAAGAAAGTTGGAAAAACCTCAGTAGATGGGAGAAACCCCGGATTAGACACAGCCAAAGA
GATAATTAGTGGACTGGAAGAGAGCTCTGAGATATTTTTCCAGAGAGTGCCCAGAATAATAAAGAGAAAAATATGGAAGAGTAGTTAAAAGACA
TGAAGGATATATTGAGCTACTCCCATAATTTTTTTTTTTTTTTTTGAGATGGAGTCTTGCTGTGTCACCCAGGCTGGAGTGCAGTGGTGCCATC
TTGACTCACTGCAAGCTCCGCCTCCCGGGTTCACACCATTCTCCTGCCTCAGCCTCCCAAGTAGCTGGGACTACAGGTGCCCGCCACCACACCC
AGCTAATTTTTTTTGCCATACGTGTTTAACAGAAATTGTACTGAAAGAGAATGGAGAAAATGGCCGAGAAGCTGTATTCAAAAGCCTAATGACT
GAGAATTTTCCAGAATTGAAAAGGATATGAGTCTTGGAGTTGAAAGGTGTTCATCAAGTAGTAAGGAGAATTTCAGGAAAGTAAATCTTCACTT
CAAAACCACATAGCAAAACTGCAGAAAATCAAGAGTAAAAGGAAAGTCTTAAAATTTACCACAGACAGATTATCCACAAAAGAACAACATTAGA
CTGACAGCTGTTGCTTTTGCGCCGATGGCAGTCTTTTTAGCAGTCTTCAGAGTGCTGAAAGAAAATAAAACTTAGCCTAGAATTTCATACCCAG
CTGAACTCTGATTAAAGAGTGATGATGAAATGAAAGAGAAACCAGCCAGCTTCAAACCCTGAGGCTCTTGAACATTTAGAGGTTGGGGAGAGGA
GGAACCCTCTCAAATCACATATATTCAGTCATCCAGCAGTGGCCTCTGTCTCCCACAGAAACTGTAAGGAGAGAAGGATTGGTTTAGCCTTTGT
AGTTGCAGTGCGAGGCCAGTGGAAAAGCCCCTTGTCAGCCATGTTGCTGAGTATTCCATCAAGACAGAGGAGGGAGATGGTCCCATCTGCTTCC
CATCTTCCAGGAATTCAAAACACCTCTGAAGGGCTTTGTGTGTCTAGGAGAATAGGATCTCCAGTGTCTCTAGAAGAATGGGAATGGGTGGAGG
GCAGGTAGCAGAGATTCTGAACCCAGCCTGCATTTCAGTTCTGTAGCAGTGGTGACATTTCAGGGGGAGGGGGAGGGAGCAGGTGGGACCAGGC
CCTGGGCCCTGGTTGGGAAGGAGCCCAGGGACATGAGAACCCCTCTAGAATGGCCCATCCTAGACTCTTTCTCTTTCTTCCAGATTGTGGACAA
GAACAACCGCTATAAGTCCATAGATGGCTCAGACGAGACCAAAGCCAACTGGATGAGGTGAGCCCTGCTCTGCTGATGTCCCGGGGGTCTTGCC
TGGCCTCTGGAAAGGAGCCAAGGGAGTGTGTTGGACCTGTGGGAGCTCCAGTGGTGGAGACTTCTGGAGGCTGCCGTTTGCAGGGTTTGGATGC
ATCTAGCTCTGAAGGACCCACCTTTTCCCCTAGGCCTCCATCAGGGGGCTCTAGAGGACGGCGTGTGGTCAGGCCGTTGGGATGCAGGGACCGC
TCGGGCTGCTCACTTGCCAGTGTGTCTGGAGGTCCTGGGCCCCAGGCTCCTGGCAGCATTCCTAGGGAGGAGGAGGAGACTGTCCCCCACCTAG
GACTGTGACACCTCAGAGCAGGCTCCTGCAGGGGTGGTGTGAGAATCGTCCCAGTGCAGCCCCAGCAGTTGTTTAAACAGCCTCCATGGAAGAA
AGGGCTCTGACAAAGTCTTCAGCTCAGTTTTTGACAGTGTTGCTGCCTATGTTTGAGTACTTTGAAGTGTCAACTTTACCCTGTTCCGGTGTTT
GGGACAGAATTTTGCACCAGAAGTTTTCTTAAATGCCAACCAGAGGAGGCAGGAGACTGGCCCCAGGCAGGGTCACCCAGGGATGGCTGAGCTG
AGCCCAGAACCATGGGTCCAGCGTGATGCTGATGGTCTGTGCCATGTGCGAGGGACTCTGCCTGGTGGGGTTTGTGGACAGGGGTGGGAGATCT
GACATGCCTTGTTGCCTGCTTGGTGGCCTCTCTCTGGGACCTAGGAGCACTGGCTGGAGATTTGGATCTGGGTGGGAGGGGAAGGAAGCCTCAG
ATGTCTGGAGTCAGGGGCACAGCCACACCATGGGCCTGGGGCCCTCTGCCGGCATTTGCACAGTTTTCTGACCCATGGGGCCTTGAGGCTGGTG
GTGGGATTGTACTTGGAGAGAGGGCAACTTCCTAAAAAGGAGTGTGCTGTCTTAAACTCGGAGACTTTCTAAGCAAATGAGGAGAAGCAGGTTT
GAGGCGTAGAGTATGTGATGAGGGCTCCTGTGTCCTTCCCAGTGGCTCCACTGCCCACCTATACTCCTATCAGAGAGTGGCTGAGCAGCAGTTC
ACACCTCCCAGATTACTTTAGCGTTCGCCCTCTTATGGGTTAGGGAAGGTGTTCTCATTGCCGTTTGATGGGTGCAGAACAGAGGCTCCAGAGA
CCAGCGGGCCCCCTGCCTTGCAGTGTGGAGAATAGCACCGACTGCCCCCAACCTCCTTGGACACAGCTGATCCGACACTGCCCTGAAAGTGGCC
CCAGGGAGGTGGCTTGACACCCTGACTGCCTTCTCTTCTCACTGCCACGGGGTCCAGGTCAGCTGAGCACTTACAGTTCATCACCTGTGTACTG
AGCACCTAGTTTGAGTCAGACTGTGAAATGGGTACCAGGGTGGATCACCCAGCCCTGCCCCACCACACCCCATGCCCCACCCCCGCTGGCCCTT
CCCGGGCTCACACCTCCTACCACAGAGGTGACTTTCCAGTGTAGTTTTTGCACCGAGGCACAATGTGCTGCAGAGGCCCAGATGAAAGAGGAGG
GGTTCGTTGACATGGGCCTTCAAAGGGGCCAGGATTGTGTCTGGGGACACTGGGTTGTGGTGTCTAGTCAGGCTGTTGGGCGGCTGCTCAGTTT
GGACCCCAAGCCTGTGCTGCTGTGCTGCCTGCCCTGTGGCTGCAGCCTGGCTCCAGGCAAGCAGGGGAGACCGTGCGTGTGTCCACTTGGGAAT
CAGGCTTTCTGTGCTATTGCTTTTAAGGCGCCGGGAGCTTTGCCCATAGAACCGAATCTGTTCCACTCTTTCACCCATCTCTGATCTTTCCCCC
TGGGGACAAGGCCTTCTCAAAACCCCAAAAAAGGGGAGCTATCATCCAAACTAACTTATCTATCCTCTGGCACCCCGCAGATTGTGACTTAATT
GGGCTGGGGATTGGCCTTGGGCCTTGGTATTTTTAAAGCTAGTGATTCTGATGTGCCACTAGCATTGAGAACCACTGCTCTGTACAGATCTTCA
TGCCACAGTGGTCAAGCTGAGAGCAGGGCAAGGTCTTGCTTAAGAGCATACAGTCAGGGTAGCAGAATCAGGATTTGAACTCCTGGCTCTGTAA
TCCTAGGTCGGTGTTCTTCCTGCTTGTGCACAAGGGACCAGGTGAAGCGTGGCTTCCTGGGCCTGGATGGGCACTGGTGAGCAGGGGTTTGGGG
GTTTCTGCTACTGCCACCTTGCTAGCATGGTTTCTGTCCTGTTCTGCTCCCAGCCTGTCCTGATTCAAGATGGCATCTGGGTAGTCACAGCTGG
TGGGCTCTCATCTTATGACCACTCAAAGAGCAGGTTTGGGTTAGGGATGCAGTTTGTTGCCACGGAGACGCCCCTCCTGTTGCTGTTGGAGTGA
CGGGAAAGCTGTTAATCCTGGGAATCCTCCTTTCCCTTCATGTCCAGCTTCCGTGGTGTGGCTCTTTCTGACTTGCAGATGCTCTCCTTTGCAC
TGTCATGGCACCGCTTGGGGATGATTGAATGGACTCTGTTAGTTTCTTAACAAAATTAATGGTGGGCCAGGAGGCGAATGGGCACTGTCGTGTT
GAGCTTATCTTTATGACAGCCCCATCTAGGTGATCTGTCTGTGGTGGTTCTCAACCGAGCACTATTTTGTGCCCCTGGGGGCATTTGGCAAGGC
CTGGAGACATGTTTGATGGTTGGATGCTATCAGATAGAAGCCAAGGATGTTGATAAAATCCTGCATTACACAGGACACCCCTTCCCCCCAACAA
AAAATTTTCTAGCCCCAACTGCCAATAGTGGCAAGATTGAGACACCCACATTAAGTCCTCCTGATAGCCCTGGACAGTGACACTAGAATTGTCA
CCCTAGTTTTATGGAGAAGGAGACTGGGTTCACACGAGTCCCATGGCTTGGCCCAAGGAGACTCAGTCTCTAGTTCCAGGTCTAGTCCCATATT
CCTGCCACCATCTTGGACTGGGTAATGAGGCCCCCAGCCTTCTTCCCATCTTCTGGGCCCTGGTGTTTTGGGTGCTTGAGTAGGAAGGTGAGGG
CTGAAGGATGTTGGTGCCGCCGGCCTTCCCCTCTTGGTTTCAGAAGGAAACCAAGGAGTGAATGCGCTCTGAGCTTTCCTCCATACCCTCATGT
CCTCTCTTGGGTGGATTTTTTAACCTTTTATCACTAGTTAAAAGGGTGACAGAGCCACGATGTGGAGAAATCAAGTTCCAAAGCCAGAGCAGGA
GCCAAGTGGGATGGAAGGGCCATGCTGTGCCCCTTGCCCAGGGCCTCTGGCTGCTCAGTTGAGAGGGAATCCCAGGTCCCCCTGGTGTCCACCA
AAGTGGAACTTGACTCCCTCAGGACAGTCGTGGGCTTCTCAAATTCTACCAAAAAGTCACAGCAGCCCACCCCCGAAGAAAGCACTCCCATGGG
AAGGAAACCTGTTTGCCTGCACATATGCAAAGGTGGGGGCTTGGAGAACTGACCGGGACGGGGAAGGGGAAGAGAGCCACTAGGCAGAATAAGC
TTTCTGTGGCCACACCCTAATCATGGCATTGTCACTGCTACACCCAACGGTAGCCAGTGTAAATAGTTTCCCTGGTTACGTATTCTTCCGAGCC
ATTCATTCAGCTGTAGGGCTGTAGGTAAATATCGCTTTATAGAGGCAATGGGCTAATCCTAATTTCAATAAGATCCTTTACTGCTATCTTTAAG
AGATTTTAAGCCCCTTACATCATGATGTTTTTCCCTCCTTAGTAATAAACCCGAAGAGTTGTGGTTGTTATTCTGGACAGGGAGAATAAGGTGT
GAAAGGTTGAACATGAACTGCCCTAGCTCTCACCGCTAGTAAGAGCTAAAGCAGGCCCTGAAACCAAGGCCTTCGACTCTGAGTTCAGTGCTCC
CTCCCATGTAACGCCGTCTCTTCTTTCCACCCTCCCCTCCGAGGAGTAGTGGAAGGGACCTCTCAGTAGGAGGAGGGTGTGGTGGGGGCCAGGT
TGCCCCAGGTGCCTCCTGGCTCCCCAGCTGGGGGCCAGCAATTAGGAAGGAGAACCACCTGCATTGACGGTCATTTACCCAATTGTGCTTTGTG
GGATATTCAACCCAAGGAATGTGGCACACCTGGCTGAGCGTAAGAGGAAGCCCAAGTTCTCCAAGGAGGAGCTGGACATTCTTGTCACAGAGGT
GACCCACCATGAAGCAGTGCTCTTTGGGAGGGAGACCATGCGGCTGTCCCATGCTGACAGGGACAAGATTTGGGAAGGCATAGCCCGGAAAATC
ACCTCCGTCAGCCAGGTGCCCCGCTCCGTCAAGGACATTAAGCACAGATGGGATGACATGAAACGGAGGACCAAGGACAAGCTGGCCTTCATGC
AGCAGTCCCTGTCGGGCCCTGGGGCCGGGGGCCGGGCCCCCACCATCGTGCTCACGGCCCACGAGAGGGCCATCAAGTCGGCGCTGCTCACGGC
CCGTGCAGGGCGCGGCTTCCCCAGGGCGGAACTGGATGGCACCGACAGCCCTTCGACCAGCTGTGAGTATCACCAGTCCCCCCCGCCACCCAGC
CGGCTGCCTGCCCCCCAGCTTACAGTCCACAGTCTCCCACCGACTTTCCCTCTGGTTCTCTCCTGTCCCTCTCTGTCCAGCCCACTCTTTCCAT
CCTCCCCTCCTTCTCCCACTCTTCTCCTCTCCATCCCTTTTTGCATTCATTCTCGAATATTTACTGAGCATCTCCGTGCCAGAGCCGTGCTAGG
AGCTGGGAAGACAGCTGTGTGTAAACCCCACGCAGGCCCTGCTCTTGGGGAGACCCTGGACTCCTAGGAATAAACCAACGTTAAATAATTGCAC
GAGTAAACTGGAAACGAAGGGATGGAGGACACAGGAGTCCAGGAGGTCCCGAGAGCAGGGCTGACTTCATGAGCATGCAACCTGAACAGTCACA
GGGGCCGCGGGGTTTCAGGCTCTGCTGTCGCCATCTGGACATTCTTCATAGTTTTTGAGCCAGGGGCCCCACGTTTTCATTTTGCATTTGACCC
TGCAGGTTATGTAGCAGCTCTGTCTGAGAGTTAAGGAGTGGGTAAAACTGGCTCCACTGGGGACTGGGGAGGGCTCCCTGAAGAAGTCCAGCGG
ACTAGGATGTGAAGAGTAAGAGTTAACCAGGCCAGGCAGCAGCGTGGGGCGCGGAGAAGGGGAGGGGAGGCGCGCAGGCAGAGAACAGCATGTA
CGAGGCCCCGCATCAGGAAGGCGCTTGGCACGTTCCAGGAACAGTGAGAAGCCAGGAAAGCCCAAGTGTAGCAGCGCGGGGGAGGGGGCTGGAG
ACGAGGCGGCCAAGCTGTGAAGGCCCGGGCCGGGCCATGGAAAGATCAGTTCCTTTCTGTGTCTGGTCCTGACTGCTTTGTTCCTCTATTATTA
CTCCCCTGCCTGAGCCACGCTGCCTGCCCCCACTTTTCTGTGTGCTGCCTCTCAGCTCCCTCTCCATCCTCCTGGCTGCTCCGCCACCCGCTCC
CCACCCCTCTTCTCCCTCTGTACTTGTCCCTGCCTTTCCTCTCTACTTCCTGTGTCTCACATGCACTTTGAAGCCAGGGCCCCAGCGGCCGAGG
CGGGTGTCCCTTGGGGCTGGAGTTCTAGTTGGGGCTCAGCAGAAGGGGGAGCACTAAAGGCCTCCGCAGGGACTGCAACCCTTCCTTTCCACTT
CTGCACATCTCCCCCGTGCCCCGCCAGCTGCAGTCCCCACTAGAGGCCGTCTGCCTGACCGGCTCGTGAGTGGTGGGAGCCAGGCAGGAGGGGC
CCCGGGGAAGCAGCCGTGGCTCAGCCCATGGGAAGGTGCTTTGCAAAGACTCACTTGCACCTTCCCTTCCCGGGCCCCCTGCCATTGTGATCCA
CTTGCTTCCTGTTTGGCTGGGGCTCCTGACAGGTGGCAGTTCACAGAGTCAGATGTTTAGGACTGGAAAAGAACATGGAGACCATTTAGTTCAA
CTTCCTTGCAATGCAGAAAGTAAGCTCTGAAAACCAAGGTGGCCCTGAGCTCAATTCAGAAGCAGGACTTGAACCCGGGTCTGCTGGCTTCCTG
TGAATCAGTGTTTTCCTAAGTCTTAAGAGGATTCCACCCCGTGATAAGAAAAAATGGGGGGGTCAGCATCCAGTAAAGCCTGTTCCCTGCATCC
TGCTCCGCTCACCCCTCCATCTCCCTGAGACTGAATGTAATGAGGCCACCAAAGGTAACCTGGGCTGGCAGGGAGCCCGAGTGTGCCATCAGGC
AGAGCCCGCTCACCCCCATGGCAGGCTCACAGCCCTCCTTTTCCTGGCAGTGGCCAGCTGCGGCAGCACTCGGCCAGGGGCTTGGCAGAACGGG
GACCTGGGAGGGAAGGAGCAGCATCACCTCGCCGGGTCTCACTCCTTCCAACACTGGCCATGCTAGACAGGTCTCTGGGATCTCAGGGGATGGC
TGCTGTCCCACCAAACACTGCCAAGAGACACAGGACTCTTACCAAGCTGGCCCAAGTGAGCCCCCAGGACCCCTGGGAGGCATGAGGTAACTGT
AAGATAAACCTGTCTGTCCTCCCCATCACCTTCCTGGGGGGTGGTTCTGAGTTGAGTCCGCAGATCCAGGCAGGGAGGGCTTGCCTGTCAGTAG
GGTCAGCCCATACATCATTTAATCCTGCCACAGGGATCAGCCCATACCCTTGGTCACCAGAAAGCAGGACCCACATGTGCAGAGGCTGGGCCCC
CTCCCTGTGTCCGGCTGCAAGGAGCAGTCTGTCCGATGGCCCCTGTGCGGTCAGATGCTCCTGGATGGGAGCACGATGTGACTGAACTGGAAGC
CTGCCTTGGGGGACAGGGGGACAGCAGGGCTGGTTGCAGAGGTGGCTCTCCTGCTCTGCACTGTCCTGCCAGTTTCTTCAGCCTGTCAGCATGT
GCTGCAGTGATTTAAAGATGCAGATTCCATATTAGTAGTCTTTTATCCCTCACCCCCTTCCCACTCTTCCCCCCAAGTCCGCAAAGTCCGTTGT
ATCAATCTTATGCCTTTGTGTCATATGGCACAGTGTATACTGCTTGGGTCATGGGTGCACCAAAATCTCACAAGTCACCACTGAAGAACTTCCT
CATGTAACCAAACACCACCTGTACCCCAATAACTTATGGAAAAAAAAGTGTGCAGATTCCAGCAAGGCCACAAGATGGCCCCAAGCAATGCAGA
CGTGGCCTCAGAAAGTCTGGTGCTCAGAGGGCCAAGAGCGTCTTAGTACCTTTAGGCAGGTGCCGCTCCTTCAGCGTTACAGAGCACCTTCATG
TTTGTCATCTTGTTTATGACTCTTCTTAACAACCCAATTAGGTAGCCAAGCGGTTCGATGTGTAGACAGGGCAGTCGTTATCTTTGCAGAAAGT
GAGGTGCAGAGAGATGAACTGACTGCTGAGGTCAGGAGCTGCTGGGTTGGCACGCAGCCGGTGGGCCACGGTCTCCAAGTCCCCTGGCCTGGGC
TCTTCCCCACTAGAATAGAGCTGTTCCGTTGAGAGGAAGAGGAACTTCCCCCCAGGGATGCTGGGCCCACGCACCTCAGGGATTTGGGGAACGT
GGATTTCTCTATGGGCGGGGGCCAAACTAGACCGCAGGGTGGCAGGGCAGGGGGATACTCAGGTGTTTTTCAGGGGCAGGGCCAACCCCTGGGC
CTCCCTCCTTCTCCCTTTTGTAACCACTTTTCCATCTTTGTTTCTGTCCCCCAAGATGATGAAGATGAGGAGGCGCCTGGGCCCTCAAGGCAGC
CTCTTCGGGTGCCTCTGCAGCGGTCTCCGGAGGAAGAGGCCCACCTGGCCAGGCCCGCCCTGCTCCGTTCATCCTCCTCCTCAGACCAGTCTGA
GACGGTGGGCCCCAAGCCAGAGGCCCTGCCCCATCCCTCGCCCCAGGCCCAGGCTGCCTGCAGGACCCCTCGGCCGCACCCCAGCCCACCCACC
ACGGGCCTTGACTGGCAGCTCCTCCACGTCCATGCCCAGCAGACCGAGGTGTTCCGGCAGTTCTGCCAGGAGCTGGTGACCGTGCACCGGGACA
TGGCCAACAGCATGCACGTCATCGGCCAGGCCATGGCCGAGCTGACCAGCCGTGTCGGTCAGATGTGCCAGACGCTGACAGAGATCCGGGATGG
GGTTCAGGCATCTCAGCGGGGGCCAGAAGGGGCAGACCCTACGGGCTCCACTCCCCAGGCCACCCAGGCCCAAGCCCCCCTGCCAGAGCCCCCA
CCAGCTTCCCCAGCATCAGCCCCCACACGGACTACCAGGTCTCGGAAGAGAAAGCACAATTTCTAACCCAGCTAGTCTGCACTAGGAGGAAGAG
TCATGGAGGAGGGATGTCTGGCCTCACAACAGGGGCCAGTCTTTCGTGTCCAGGAACAGGATCGATGACCCTTGTAGGACACTGGGGGCGCTGT
TACCTCCTCACCGAGCGCAGGTCGCTGCCCTGTGGTCTAACAGAGCATGTATTCAGACCCGCACCCTCATCTTTGGCGGATGCTGAAGATGCAA
GAAAAGCCACCTGCTTGATGCTTGCTTGGCCTGGAGATCGTTCTCCTCTTGGTCTGTGTCCCGTGGCGGCAGGCCTTGCAGGGAGGGGGGCGCA
ACAGTGGGGAGCATCCGAATGGAGTCCCACTTTCCAACCTTGGGGCTGCTGCATCGGCCTCCCACATGTGACCTCCCAGGCCCTGATGCTGCTT
CTTTTGAAGGGGGTGAGGCCTGATGGGCAGGTCAAGTCGGTCCTTCAGGGAGGGGTCTACCCTCCCTTGCCCGGGGGTGGTGCCCGCCTTGGCC
CTCCGCTTGCCCTTGTGCGCGTGCTCAGCTGCTGTGCTTGCCTTTTTTGGAGTGGGGAGAAGAATGGGATGACGTCGGAGGAAGGAAAAGACTG
TCTTGCATTCATAGAGAGCACTTTATGCTTTTCCTCGCCCTTGCAGAGTCTGGGTGGTCTCAGTGGGGGCTCAGGACAGTCAGCTCAAGACAGC
CAAGTCAGGTATTATTATGATCATTTTTATCATCATCATCTGTATAAGACAAATGACACAGCCAGTTAGTGAGAGGGCCAGGACTGGAAGCCAG
GGTATCGGTTTTCCCAGCTCCCCTGTCAGAGGCCAGATCTGGAGTAGTGGGAAGAGGAGGCAGGGGGCAGGTCTTGCCCCTTGCCAGGCCTCTG
CCCCTTGCCAGGCCTCTGCCCCCTGTATCTCTGCTTCTGCCTCAGCTGTCCTGGGTGAGGTGGGAGGCACCTAGCATTTAGCAGGTGATTCTCC
CACCCTGCCCTGGCTCTATGGCTCATAGCCAGTGGCTTGGAGAGGCAGGGCCAGATCTCAGGAGTGGAGGGCAACCTCTTCCCTGGCTTCTAAA
AGAATCCAGAGCCCAGTGGGGCTCATGTCCCATTCCAGGTTCCAGCATGCCTTGCCTCTGCTTGCTCGGGCTGCCTACGCACTAGCTACATACA
CCTCTAGCTTCATGTTTCATGTTGGAGTGTGGGGAGAAAACTGGGTTCTTAGCATTAGAGAGGGAAGTGGCAGGTGGCAGATACAAGCTTAGCT
CTGACCTGGGTGCACCTCAGCTCTGTGTCCTTTTTTTTCCCTGACCTGAAGCTGACCCCAGCAGAAGTCCCCCAGCCAACCCCTCCACCCAACA
ACCCCCCACCCTCCCATCCTGCCCCCAGCCATAGTGGGAGCTGGGAGGATTGCAGCATCTAGTGCCTTCCAAGGCTGTTCCTGGAGCGCTGCTT
GTAAACCCAAGCCAAGCTTTGTCCCACGTAGGGAGAATGGAAGGTGACTGCAATGTCCCTGTGCCTTTGGCTCCATCTCCCCATTCTGTGGTTC
TTTGGGGGTTCCTGCTTCCCTCAAACTGAACACTCTCCTGTCCAACAGCCTGATGGCTTAATTATTGCCTGGAATTGCCCGGCCTCCGATGCTG
GAATCAAAGATTGCCTTCCGAAGTATTTTTGTTAAGATGGCAATAAAAAGAAATCAATCTCACTCTTTGAACACACCCAGTGTCCAGGATCTTT
CTTTGGTGTGTATTCTCTTTCTTCGTTTCCTGTGAAAACCTCGAGGACCCCAAAGCAGGGGTTAGATGTTTAAGGAAAAGCAGGTTGGTGAGAA
GGGCTCAAGTGCCCCTTGATTTCCCTGATTCTATAAACTGCCAGATTCCCTGTATCCAGCACCATGCCAGGCCCTCGGAGGAAAGGAAGGGCAT
GCATGCACGAAACCACTCAGTTATATTTCATTTCTCTGCTCAAAAGTGAGGTGTTGCTAAGTCAATTTTACCTTTTGTTCCCTAAAAGTAAGCG
AGAAGTTCTGGAAAAACAATCACCGTTTCGGACAGGAGGCTCTAGAAGGGTCAGGGGCTGTCCATGTGCCAGTGCACTTTAGGGGCCACCTGGA
GGCATGACCCTCCCCACCAATCCAGGTGTCTGTGTGTCTGGCACTGGCAAGGCTCTGCCCACCTAGATCTGAGGCTTCTCAAAGTAACCAAAGC
TGCCTCCTCTGCCAAGAGGAAGCCTCCTCACCTGTGCATTGCCCTGGCTTAATACCAGTCTCAGAGAGCTTCTCTCAGGCAGGAAATCCCCTAG
TCTTGCCCACAGATCCCCTTTCTAGAAGGTTCTAACTTGCCATCCTCCACAGTTCTTGGCCAAAAAGCATGGCCATAGAAATGGAGGACTACCC
AATTTCCGAGAGATGCAAAGATGTCGTGAAACCATTCCCAGGGCCTTCTCTCCCAAGGTCAATGTATGAAGCAGTGACTGGCTGGCTGCTCTCT
CTCTTCCTGGGGAGGGAAGGAGCCAAATTGGAAGCTGTAAAACTGAAATGGAAGTCAGAGCAAAAGGGACAGTGAGGACTTGTCAACTTGGGAT
GGAAAAGATTTTCAGTTTCTGTTTCTTGGCTTCAGTGGGGATTGTTGGGCTAGCACATTAAAACAGGAAGAGGAAACTTGGGTGAGAGACAGAA
ATGAATTGGAAGAGGGAGGCCTCTAGCCATGACCAGGTGACCATCTGGTGGGTGGATGTGTGACCCAAAGCCCCTCCTAGCCCGGAGACCTATG
CATGTGAGAACACAGAGCCCAGCCTCTTCTCAGTCGGTCTTTTCCTAGTTCTGCCTCCTTAACTGGGGCAGCTTCCTTTGTATTGAAGATACCC
ACTATGCAATAGTGTCTGAGCCTGGCCTAGTCAATTCCCTCCAGCAAAATGGATGCCCTGTGTTATGTTTTCTTTGCGTTAGAAACAATAGTCA
TGTGCTGACCCAGCTAGAAAATATGATGCACTTGATAAGACACAGTGGGTGAGGTCTGAGAATTGTTGTCTTAGTACTTACCTATCCAGTCCCC
AAGGTCAGCTGGGAGGTTGGAATAAAAACAATCCAGGATCATCATGACAGGTTTAGTCCAGAGCTCTCCACCGCCATCAGCAGCACCACCACCT
TCCCCTTGATTGCTCCAAAGGTCCCAAATTCTGTGGGTTGGAGTTGAAATGCCTCAATAAGCACTAGCATTTGCAGAACAGTCTCAGCCAGGCA
TTCTCTGTGCCTTCTCTCAAGAGGTAGTGACCTGGTAACTCTTCAAACCTCCTTGCAAAGAGCTGCTCAGCCACAGTGGATAGATTTTGCCCCT
CCTGGTAGCCTTCCACTCCTTGGGGAGGGCTTCTGATTTCTGCAGCCATGTAATGAATGGAACAGGCACAGTTCTTCATAAAGGGGTTGTTGTG
AATGGGGACAGAAGGGTTCTGGGGGGTGGTCGAGCCAGCAGGTGTTAAGGCAAAACACTTAAAAGCAGGAGATCTAATCAGAGGTAACAGGTCT
GGGGACCTAGCTGGCTTCATTTAGCTTTACCTCTTAAATGGTATAACATAAACATCTTGTCAGGGGAATGGGTTGTGATTCACCAAGAGTTGCC
ATATACATTGTATTTCAAATGCTAAGTTTCAGAACAACAAATACAACAAAATCTGAATAAGCATTGATCTGCCTTTGAAAATACTTTGTGATTT
ATACAACGCCTTGAGGTTAGCTTTCCAAATATTGATTCTTGATGGATAGAAAAAGACCAGAAGATGGAAAGTTAAATTGACTTGAGTCTTTCTA
AACCTTTTGACACACCCTAGAAGTTGCTCTTTTGAATAAATAAATCCCAGATAGGAATGTATTTCTCATCTAGCTTTTTCTCTTGATGTCAAGT
GTCAGAGAAATAATTCTAGCTTTTAGAGGTTGGTGGTTGTTTAGAAACTGGAAGAAAAAAAGGAGAAGACAGTATTCTCCAATCATAGTGCTAT
GATTCAAGTGAGTCTTGTGAGAAAAAAGACGAATGAATGGTTTCCCTTTTGTATCTGGGAGCAGGAAAGTTTGGAACAACTTCTATTCTGTGCT
AACCGCAAGGTTGATTATTTTTGGATGTCAAATCCTAGACTCTCACTAAATTGGGGACTGGAAATATTTTGCATATTCAGAAGAAATCTCAGCT
ATGGAGAAAAGACTGACTCACAGTTGTAGAATTGAGATTGGGAATTATTAATTTGGAGTGACTTCTCTCACTCCAAATTAAAGGAAAAGGGGCT
CTTTTCCTTTCTCTTAAGTCCTTTGTTAATAACATCCTGGCTGGGTGCGTGGAAATCACCCTGTGGTCTTCACTACTCAGGGATAGCGATGCTT
ATATCCGATGTGATAAGAACACCGATGTGTTGGGGCTAACAGAGAACACAGACCTAGGACAAGATGCAAATTCTGGCGGATCTAAGCACCGTAA
ATCTTGCAAGTTCTACACCTCCATGGTGGAGCCTGTGGCTTGCTTCTCTCAAAAGGTTGAAGCAGCCAAAGCCAGCATTTGCTCTCCCATCTGC
CTTGGTCTTACTCCTTCTGAGGTCTTGCAGCATCCTGTTGCCAGAATGGATTGTTCCCTGGACTCCAGTGATCCTCCTTGGAAGGCAACAGCCT
CCTCCCAGGCTCTCCAGATGAGCAAGGCAGGATATAGATGAGGGGCAGCTGGTGCCATCCCTGGGACCTCTAGAATGTAGATTCTCCATGCTGC
TAGCTGCCACCACTGTCACCTCATACTGGAATTGGCATTCGTATATTTCTTTTCTTTATATGTGTGTCAGGAGTGAGGTGCGTGACAGTAATAG
TTGCTTATTGAGGGAAACTTTGAAAGTATGCAAAATTACAAAGATGATCCACCTGTAAGGGGTTGTCACTGTTAAAACATTGGTATATTTCCTT
CAAGTCGTTTACTGGGGCTTGTTTTTAAAGCATTATTGGAACCATAGTCTACTTCCAGTCCTATTGTTCTCACACTATCTTATTTCAACACCAT
TTTTCCATGTCATAAAAGATTCTTTGAAAACATTTTTCTATAGATGAATAGCATTCCATTATATAAATGTAACATCATTTACGAAATCTTATCT
AGTATTAGACATTTAAATGTTTTGCCTTTGTACTGCTATACACAATGATACAAGTCAGCATATTGGTATGCCGGGTTTGCATACTTCATCCATA
ACTTATGTTTCTTGAGCCATATTGCCAAATTGTTTCCTAAAGGGTTGCACCTGTTAATTCTGCATCATCAGTACAGGAGACACCTTTCTTACCA
CCTTTACCAATAGAGATATTATCATCTTTTAGAAATCTGTGCCAATGAGACAATGTACTACGCTGTGTTTCTTTTATTAGTGATGCTGAATACT
TTTCATTTGTTTATTATCCATCTGAATTCTTCTGTGAATTTTTGTTTCCCCTGGACTTTTCCAGATATAATTCATTGGGCTACACCAATCAGAC
AGACTTTGATTTTTGTCCCTTGTTTTTGAGACAAACCATAACATAAAGAAGTTTATTTTTTACTACAAATTTCCTAGGTGCTTGTGTAGCTCAG
AACCCTAAAACGTCAACTAATTTCCTCTTGCCCTCTGATCTGTACACCTTCACTCCTCGGCAGGTGTTCCCTCATCCCACCTTTTGGAGGTGGG
TAGGGTGGTGGGCTGGATGTCGCACATGTTGTTTCAGTGAGAATGGGCGAGGGCCATAGATTCTGAGGGTTTGACAGAACTTTAAAGGTTTTCT
AACCCTAAAACGTCAACTAATTTCCTCTTGCCCTCTGATCTGTACACCTTCACTCCTCGGCAGGTGTTCCCTCATCCCACCTTTTGGAGGTGGG
TAGGGTGGTGGGCTGGATGTCGCACATGTTGTTTCAGTGAGAATGGGCGAGGGCCATAGATTCTGAGGGTTTGACAGAACTTTAAAGGTTTTCT
GGGCATTGCCGAGTTGGTGCTTTACATGCCCACATAGGAGCCATGAGTTACAGTGGGAAACGTGCATAGGACTCAAAGCTCCATCTCCCATTTA
TTAGCTGTGTGACTTTGGGCAAGTTGCTTGGCCTCTCTGAGCCTCAGTCTCCTTACCTTAAAATCATTTAGTAAGATTAACCAGTGGTTGTCAA
CCTTTTGTAGTCTGTTATTTACCTTCCTCCCCTGTCCCCACTGGAGCCCCCATTTTTTAAAAGATTTTGATCTTTAGCATTTATTAATTGATCT
CTTGGACACCTTGTCACAGGGGCTATTTCAGCAAGGCTTTGTGAAATACTGGAAACAGTTGAAGGACCCCAGCCTTACCACCTTCAGAGGCCTG
TGGGTATCTGGGAACCCCAGTGGTTAAAAGTTAGTGAAGAGTAGAAGAGATAACCTTTCAGTTTTTCAATCACTTATAATATTTAGTTGATATA
TTTTCCATATGGTGTGAGGCAGGCAGGGCAGGTGTCATATCCAGTCTCAGAAAACAGAGATTCTTAGAGGTTTGGAGATCTGCTTGAGGTCCCA
CAACACGGTGACGTTGGGACAGGGATGGGTTCTGGCTGGTGCTGCTTCTCCGGACAGGGCAGCTGCTCTGCTGATGTTCACATGGGCCCACGTG
CCTGTGGACTTCTAACCATCCACACTTGCCCAGCACTGTGCCGGCACCAGCGGGCACTCAACAAAGGGTGGCCCGTGCGTTCTCACCTGTCTCC
CCTCCCCACCAGGTACGTGGTCATCTCCCGGGAGGAGAGGGAGCAGAACCTGCTGGCGTTCCAGCACAGTGAGCGCATCTACTTCCGGGCGTGC
AGGGACATCCGGCCTGGGGAGTGGCTGCGGGTCTGGTACAGCGAGGACTACATGAAGCGCCTGCACAGCATGTCCCAGGAAACCATTCACCGCA
ACCTGGCCAGAGGTGAGTGCCATGCTCCACATGAGCTGCGCCCACCTCTGAGCCCCAGGGGAGGCCTGGATAGCTTGTTTTGGAGCTTCCTGAG
AAATACGGTTCCCAGCAATGGGAAGACCCAGAGTGATTCGGGGGAACAGATGGTTGAGGTCTGAGCAGCAGCTCTGGGCCAAGCAGGGGCCACC
CCAGCATGTTATCGACCCCTAGAAATGGTCTCAGAAATCCTTACCACACACATTTCAGAGTATTCATGAGAGAGACGCCCCCAAAACCGAGCAA
ATATGTTTAATCAAAATGTACCCTATTTCCCCAACTCCAGTTGATGAAAAGATGTTACAAACTGCCACTGCCTCAATAATGATCGTAATAATAG
GTAACATTTACTAAATACTCAATATGTGAAATACTGACAACAGTCCAAGGACCCCAGTCTACCACCGTCAGAGGCCTGTTGGGTATCTGGGAAC
CCCTGTGGTTAAAAGTTTGTGAAGAGTAGAAGAAATGACCTTTCATTTTTTCAGTTACCTGCAATATCTGGTGTTGATATATTTTCCATAGCCT
GTGAGCAGATTTTCTGCATGTTCCCATGTAATTATCATAGCAGCCATCCCTACCCTGTCAGTGCACCCTGAGAAGTAGATGGAGTCATGGACAT
GTGGCATTTAGGACAATGCCCACCTCCATCATCAGAAGTCCTCAGCTATAAACTTTTAATACTCATGATCCCACATGCATTCTTTTACCCATCT
TACAGATGTGCAAACCGAATATCAAATGAATACAGTGATTCATCCAGGGCCACCTGGCTAGGAAGGACAGAACTAGGTTTCTAATCCATATTGG
TCTGACTTCAAGGTCTAAGTTCTGACCTGCAATGTTACCTGATGTCCCAGACAAAACCAGTCCCCTCCTGGGTTCCTCCTGGTGTCTGTACTGC
AGTCCTAATGAGATGAGGTGAAAAAACAATCTCTATCAAAAGAAGAAGGTTGGCAGGAGGTGGGATGGTGACTTAGGTGTGGGAAGCCGGGTGG
GTTCGGTGTCCATCCAGAAGCAGTTTTAACTACTCTCAGCCCCTAGTCCTTTACCCTTTTATTCCATCCTCATCCTGACCAGTGTAGACTGGTG
AGTCCAGGCCCTTCTTTCAAGGGTGTTTTTTTTGTTTGTTTGTTTGTTTTTTTCCCCACTAAAACCACGCATCCAAACACCTAGAGTCTATCTG
GTGCCAGGAACCAAGGCTGTTCCCAAGCCTCACCATATCGTGTTCCTGACCAACTTACTAATTGGAAATAAATCTTCACTTGGAATATGAGGGG
AGAAGATGAAAAGGGGGTGGGAGTGACTTATTCAAATTAAGCTATCTTTAAAAGCAAAACAGAGCAAAGCAAATTGTGGACATTGAGTGCACAG
CTGCTGCCTGACTTGGTAAAAGGTATCTGGAGATGATGGGCAGTAAAAGGAGACCCCATAAACATACTCATTTCTTGTCTTCTTTGGATCTCAG
GGTTTGGCACATGTTGGTTTCACTTGGCAAGGTGTGCTTCCATGATGAGGCTGCAGTGGTTTCCATGTGACCTCTGGAGATGCCTCATGTAAAA
GTGCCTGATACATAGTAGGGACTCAATAAGTCTTTGTTGAATGGATAAAAGAAGAAATGAATGAAGACAGCCAAGAAGCTTTCCAAAGAATCAT
ACCACTAGAAGAAAAGAAAACCCAGAGAAATCATATCTGAGAACACAGATTTCATCCCTTGGTCATAACCTTCTTGCATTCTTCTGGATTTGTG
ATGTCCTTTTCTTCCCCTCAGTGGTCTTCTCCAGAGCACCTGAAGCTGCATCATCCTCCATGAGCCCCAAGACCACAGGGGATTGCTCAGAGAA
GGGTGTGAGCTTTCTGGAGTGGTTCAAAGGAGGCGTGTCAGACAGACTTGAAAGGAGATACACCGAGACATATGAAGATACAGGAGGGAAGAGG
GTCTATTCCTGGTCAGGCTGAGCAGTAAGAAATAGGAAGGAAATTGAAAGTCACAGTGGGGCAGTTTCACAGCTGTGGGTGATCCTGAAGCCCT
TGGGAATGGTCATGGAGCTCAGTGGCTTTTAAATGTGTGCCTCTATAGCCTGTAGCTTCAGCGGACTCTGGTTAGTGGTGTCCCAGATGGGTGT
GCCATTGCTGCTCTGAAAGGGAGATCCCCTATGCCCAATCACTCTTGGTCTTATATAGAAGGAGGTTTCTTTGTAATTGGAGGTTGGCACCCTG
ATGGAGTCCAGAGCATGAATTATTAGTTCTTATTAAGTAGGGATATTGGTTGTTGCCAGAAGTCTTCCTGAAGAGAGTGTAAACTATTCCTTTG
TGGTTACCTTTTAAGAATAGATTGCATCTAAAGCTCGCATTCAAACAATTTATGTTATTTGACCTACTTTGATTCACTAATTTACTTTGAGGTT
TTCTTTGTTTTTGTTTTCCATGTTGTATGGAGGCTTTGGAACCAATTTCATGATGCTTAAAGGTCTCTTTTGGCCTGTAGATTTTTCTGAAAGC
CTTAAGTCCACAAAGATCATACTAAGAAATTTGAACAACGTTGTTTTCAAACAATGACGAGGAATCTCTGGTGATTTCTAGGCTTGATTTCCCG
ATGTCCTCAGTTGTTTGCTGCCTGATTGTCCCATGAGGAAAGGAAAATGTGGCTCTTGATTTTTAGAGATTTTCAATGGGCAAGTGCTGCATCT
GATCTGTAAGACATACAGGTGGCATTTTCAAGTTATCTCCATCTCTCCCCTTTCTGTAGCTCAGCTGATATAGAAGTGCAGGCTGTGGGGAGCT
GCCTGGGTTCCAGAAGCCTTGGAGATAAAGTTGTTCCTTAGGTATTCATGTGAGGACAGAAAAGGTGCATCCTGAATAGATAACTCCCTTTTGT
GCTGTAACCTAAAGGAAACCTGAAGTCAAACATGGGGCAGGGCACAGCACCCATACCAGCTATGGGAAACCAGACGTGGAACATCTGGACTGCT
TATTGGCAAACCCTTGGCCTTCAATCAGAAGTCTTTTGCAAATGGAGCCATCAGAAGCCTAAGTACGTTTTAGTTCAAGTCATTGTTCAGCGGC
ACATCACCTAGGGCCCCTGCACCTGGTCTAGGAAACCTTTGAGATTTCTGAGTTCCATAGGCTACTTTCAGGACCCTCTAAGGGCTGAAGAGAT
TCCTCTGCCTTTTTAGCATCTCTCACCAGCAAGCATCAGCACTTCTGTGGCAGTTTATGAAACTATGTTGGTAATTTTTAAAGAATCAGGCTAG
CTGGGTGCCATGGCTCATGCCTGTAATCTCAGCACTTTGGGAGGCCAAGGTGGGCAGATGGCTTGAGCCTAGGAGTTTGAGACCAGCCTGGGCA
ACATGGCGAAACCCTATCTCCACAAAAAGTACAAAAATTAGGGTGTGATGGCTTGTGTCTGTAGTCCCAGCTACTTGGGAGGCTGAAGTGGGGG
GATCACTTGAGCCTGGAAGGTGGAGGCTGCGTGAGCACCACTGCACTTCAGCCTGGACAACAGAGTGGGTCTCTGTCTCAAAAAATAAAAATTA
AAAACAAGGAATCAGTCTAAAATAATTTATGGTTGAGGAGCTCACCAAAGTCTTTGAAACAATTGAAAGTAATTCAAAGTGAATTGAGGTAATT
CACATGATAGAAAGAAATAGGCAAGGAAACGTCCTTTGAATTGCCAAGTGAGGAGACATGGCTATATTTCCTGACTGCTTTGGGTCATTATGGC
TACCTTGTCCTTTATCTTGTCGGAGGCTGCCATGTTGGAGCCCTCAGCGCCATAGGTCTCCTTGTCTTCCCCTTTCTTCTGCCCTTAGTCGTCA
GTGAGAACAACAGAAGTTCAGATCATGCTTTCTCACATGTTCCTAGTCTGCTGATTGCTGGGAGAATTAGAAAGGACAGTTGCCATAGGATGGA
CTTTGCCTTAAGTAGGCTGTCTCCAGAGCAACGAAGGAGGAGGAAGGAGTGAGGCCTATGGTGTTTCATGTGTACCTTTTACCAAGTCGAAGCA
GCCATCTTGTCATTGCTAGGGCTGAGGGGAAGCTGCAAAGGTTGGGGGAGTTGATACTCACTTGGCTCTGAGGTATGTTCCCACCACCCAGTGT
GATTAAGTGGGATGTTGTTTTCTAGGGTCATAGGAAAGAATGTTCAATTACTCTCCCTTATAGATTTCTCTTAACTTATAACCGTAGAGCTTGT
AGACATGAGGCTTCTTGGAAACTGTCTCTTTCTAAAAGGTCTTTCCACCGTTCAGGTCCTATGAGTCTAGCTCTGATGGACCACTGAGTGATTC
GTATCTCCCCTTTGCAACACTTGCCCCAAAAGCCCAGATCTAGAGGGATGTGTCAGGTGACCTAACAGGAGGCCTGCTTTGTTTTCTGTTGGTT
TCTCCAATTTGGGGGTTTTCCCCATTTCCTTACACCCTGGTTTTCCTTCCTAGGAGAGAAGAGGTTGCAGAGGGAGAAGTCTGAGCAGGTTCTG
GATAACCCAGAAGACCTGAGGGGTCCCATTCATCTCTCTGTGCTGAGACAGGGCAAAAGTCCCTACAAGCGTGGCTTTGATGAGGGGGATGTAC
ACCCCCAAGCTAAGAAGAAGAAAATTGACCTGATTTTCAAGGATGTTCTGGAGGCCTCACTGGAATCTGCGAAGGTGGAAGCCCACCAGTTGGC
CCTGAGCACCTCACTGGTCATCAGGAAAGTCCCCAAATACCAGGATGACGCCTACAGTCAGTGTGCAACAACAATGACCCATGGTGTGCAGAAT
ATAGGCCAGACCCAGGGGGAGGGGGACTGGAAGGTCCCCCAGGGGGTCTCCAAGGAGCCAGGCCAATTGGAGGATGAAGAAGAGGAGCCTTCAT
CATTCAAGGCCGACAGTCCTGCCGAGGCCTCCCTTGCATCTGACCCTCATGAACTTCCCACCACCTCTTTTTGCCCTAACTGTATTCGCCTAAA
GAAGAAGGTTCGGGAGCTCCAGGCAGAATTAGACATGCTTAAGTCTGGGAAACTTCCTGAGCCCCCCGTATTGCCACCACAGGTACTGGAGCTC
CCAGAGTTCTCGGACCCTGCAGGTAAGTTGGTTTGGATGAGATTATTGTCGGAGGGCAGAGTACGCAGTGGGCTGTGTGGAGGGTAGCCTAAAG
CTCTCTGTGGAAACCACCTTCCGGGAGACCTGAGGAGTGTAACGTGGAGGCGGCTACCTCCGTGGGTGGGAGCCCAGGTCCTCAGTGTCTCTGG
CAGACCCATCGGCAGCTCTGCCAGGTGCTCCATGTGTTGCCCTTGTATCCTCCTTGTCAATAAAGGAAGTTCCGCTGCAGAAGGGGTGTGTGCT
GTGTTCTTGACCCGTTGCCTTTCTCTGGTACTGGTGTCTTACCCCAAAGCCCAATTTCTAAACCCAGTCTTTCTCTGTCCCCAGTCTCAAGCAG
GGTGTCCCACTGGAGAGATCTCTTGGCTTCCCTAACTTAGTCCAGGAACACAGCCTTGTTCTTCTCTTCCTGAATCTCTGTCCTGCCACACATG
GTCCCAGTTCCCTAGCCTGGAGTTCTGGAAGGATGGAGAGTGAGGGGATCCAGGCCATTCACCTGCATGGCTTTGCCCTATTCTGTTGGCTACC
TGGATTTCTAGAGTTGGTCGACAACTAGGCAGGTGTTCTAGTTCATATCTGCAGCTGAGGGAGACTGTTTACATAGCACTTACTCTTTTAACCA
CATCCCTTAGCTCAGAATGAGGTGTTTCTTGTATTCAAAGCATGCGTCTGAACTAAACTGCATTTTGATCCTGAAATCACTTGGGGCCATATCC
AAGATGCCTTTGTTCACATTAATGAAGGGCAAATGAATCCCAAGTCCTTGCCATATAACTTTGGAATGTGTGATGTGCTTTTCCTCCTCCATTA
GATCTACTCTCCTAGCTTGTGCTGTCCAGTTGATAGGGGGCATTTAAATCCCTCAAACACCCACACATGGAGGGCAAGGGCAGGCAGCCTGAAA
TCTGGTGGGTGTGACAAGGCAACCGTGGACAATCACAGGGAGAGTAAAAGTCACATGGGCAAGCAGGAGTGCTCATAAACTAATTCTGGGCTGG
GATTCAATCCATCATAGGCTTCCACGGACTTCTGTTTCCCATGGTGTGGCACCTACCTTCCAGGAGTGTTTCCTGTGGATTTTGGAAAAGCCTG
TTTTCTCCCCCAGGTATAAATGTTTCTTTCCCATTTGTTTTTCAGCCTCAGAAAGCATGGTCTCCGGCCCCGCCATCATGGAGGATGATGACCA
GGAAGTCGATTCAGCAGATGAATCTGTCTCCAATGATATGATGACAGCGACGGATGAGCCCTCCAAGATGTCATCGGCCACCGGGCGCCGAATC
CGGCGCTTTAAGCAGGAATGGCTGAAGAAGTTCTGGTTCCTGCGGTACTCCCCAACCCTCAATGAGATGTGGTGCCACGTCTGCCGCCAGTACA
CCGTGCAGTCCTCACGCACCTCGGCCTTCATCATTGGCTCCAAGCAGTTTAAGATTCACACCATCAAACTTCACAGCCAGAGCAACCTGCACAA
GAAGTGCCTGCAACTGTACAAGCTCCGCATGCACCCGGAGAAGACAGAGGAGATGTGTCGCAACATGACCCTGCTCTTCAACACCGCCTACCAC
CTGGCCTTGGAGGGCAGGCCCTACCTGGACTTCCGGCCCCTGGCGGAGCTGCTGAGGAAGTGTGAGCTCAAGGTGGTGGACCAGTACATGAATG
AGGGAGACTGCCAGATCCTCATCCATCACATCGCCCGGGCCCTGCGGGAAGACCTGGTGGAGCGCATCCGCCAGTCACCTTGCCTCAGCGTCAT
CCTGGATGGGCAGAGCGACGACCTGCTGGCCGACACGGTGGCTGTCTATGTTCAGTACACCAGCAGTGATGGGCCCCCGGCCACAGAGTTCCTG
TCCCTGCAGGAGCTGGGATTCTCTAGCACAGAAAGCTATCTCCAGGCACTTGACCGGGCCTTCTCGGCCTTGGGCATCCGGTTGCAGGATGAAA
AGCCAACTGTTGGCTTGGGTGTAGACGGAGCCAACATCACAGCCAGCCTCCGTGCCAGCATGTTCATGACCATCCGCAAGACGCTGCCCTGGCT
GCTGTGCCTGCCCTTCATGGTGCACCGGCCCCACCTGGAGATCCTGGATGCCATCAGCGGGAAGGAGCTCCCATGCCTGGAGGAGCTGGAGAAC
AACCTGAAGCAGCTGCTGAGCTTCTACCGCTACTCACCGCGCCTCATGTGCGAGCTGCGGTCCACGGCGGCCACCCTTTGTGAGGAGACAGAGT
TCCTGGGCGATATCCGGGCAGTGCGGTGGATCATCGGCGAGCAGAACGTCCTCAACGCTCTCATCAAGGACTACCTGGAGGTGGTGGCCCATCT
CAAGGAGGTCAGCAGCCAGACCCAGCGGGCAGACGCCTCGGCCATCGCACTGGCCCTGCTGCAGTTCCTCATGGACTACCAGTCCATCAAGCTC
ATCTACTTCCTGCTGGACGTGATTGCTGTGCTCTCGCGTCTGGCCTACATCTTCCAGGGCGAGTACCTGCTGGTGTCCCAGGTGGATGACAAGA
TCGAGGAGGCCATCCAGGAGATCAGCCGGCTGGCTGACTCCCCGGGAGAATACCTGCAGGAGTTCGAGGAGAATTTCCGAGAGAGCTTCAACGG
GATCGCCATGAAGAACCTCAGGGTGGCTGAAGCCAAGTTCCAGTCCATCAGGGAGAAGATCTGCCAGAAGACCCAGGTCATCCTGGCTCAGAGG
TTCGACTCCCGCAGCCGGATCTTTGTGAAGGCCTGCCAGGTGTTTGACCTGGCTGCCTGGCCCAGGAGCAGTGAGGAGCTGATGAGCTATGGCA
AGGAGGATATGGTGCAAATATTTGATCACCTGGAGGCCATCCCGACCTTTTCCCGGGATGTCTGTAGGGAAGGGCTGGACCCCCGGGGTAGTCT
GTTGATGGAGTGGCGAGAACTCAAGGCCGATTACTACACCAAAAATGGCTTCAAAGACCTGATCAGCCACATTTGCAAGTACAAACAGAGGTTT
CCACTCTTGAACAAGATCATCCAGGTTCTTAAAGTCCTCCCCACTTCCACCGCTTGCTGCGAGAAAGGCCGCAATGCCCTCCAGCGAGTTCGCA
AAAACCACCGCTCCCGCCTGACCCTGGAGCAGCTTAGCGACCTGTTGACAATCGCTGTAAACGGACCGCCAATCACCAACTTTGATGCCAAGCG
AGCCCTGGACAGCTGGTTTGAGGAGAAGTCTGGGAACAGTTACGCGCTGTCTGCAGAAGTCCTCAGTAGGATGTCTGCGCTGGAGCAGAAGCCA
GCACTACAGACCATGGACCACGGGACGGAGTTTTACCCCGACATTTAGGGAGCTGGCGCTGCAGAGTTCACTAAGCTGTTGAATATTTTTTTAA
TCTATACTCATAAGCTTTGATATATTATATAAATATATATTATATTATATTATATTATATATATATATATATATAAACTCACACTGAAAATTTT
TAAAAACCAAGGTGACGCGTCCACCAGAAGCCACTGGGAGATTTCAGAAAGGAAAAATGTTGGAAACTGACTCTTGTCTACAAAATTTGGCAGC
TGCAACATACATGGCAACTCATTTTCACTCACAGAAGCACGTGCTGGGGCCTCCTGTGTTCCCACCTTACTGTCCACCAACAGCATAAGCTAAA
ATGACAGGTCTCTGTCATCACCTTTAGGTAGCTCATTTTGTTTATGTTTTCATTTGCGGGTGGCGGGGCTCTGGGTTTGGGTTTATGTTCTTGC
CTTTTCTTTTTTCATTTGGTTTTATGATGGGAGGGAGCTCCTCAGCCTCCTCATTGACATTCTGGTCCGGCTGAATCAGATCTCTGACTTAAGT
CAGGGTGGGTTGTCTGTCTGCATTTGGGAGGCAGGGGGGTTGACCTTTCTCCCTCCCCACCTGACTTCAGCTTGAGATCTTTTTTTATTCATTT
CCTGATGAGGGTTCCTTCACTGTCCTACAAACAAAAGTGTCGGTCAAACTGTGACACTGCCACACCTCACCTCTGTTGCCTCGTCCATCCCTGG
GTTGTGGATCCCTTCCTTCCAGCCCCCCCTGGAAACTCACAATATTACCCATTATACTGAAGGCAACATTGCCTCACCGCTGAGCTTGAAATCC
TGGGGAAGGGAGAAGGGGTAAGCTTTTAGCATTCCTGTTTTTACGAGGTGGAGGATAAAACAATATAATTCCATTCCAATCCAGGGCTTTTGGG
GAGATGAAGAGCCAAGAAGTCCAGACCCCAACAGGGGAGTGATTTTTGGCTAAAAAACAAGGAAAATGAAAAGTACATATTCGAGTTACATGGA
TTATTTATACTTTTTCTTTATAATCATATCATGTGTTGAGGGTATTTTTTTTCCTTTAATAATCAAGAAATGCCTGCTATAGTTCAGTGGCAGG
TAGTGTCAATGCAAATTGTGCCCTAAGTTATGCATAACTCAAATGAGAGTCTGTAGAGATGTGGTCCTCCTTTTGCAACAAGGCTGATAACATG
CTACATGGTCATAGGAAACTGGGGAATGTGTCTCTGCCTGTAAACTCTTCCTTTTTTGAACAGGGTAGAGATGTCCTAAAGAAATGGAGAAAAG
AAGAGAGGACTCTCAAGGCATCAGCCTACACAGACACACACACACACACAGACACACACACACACACACAGACACACACACACACACACAATCA
CAATATACAATATAAGCTTTAGAAATAGCCACTTGCCTATTCCCTGGGGCAAGTAGTGGTTTAAACTAGAGGAGTCTGATCAATGCTCTTTCAT
TCATTTAACTACCGGTATACCTCGCAAGGGAGTTTTAAAAAATGTGCGTGAGCTGTTAAAAACTTCTGTTCATGTTCCTACATCTGATTTATGC
ATATTTTATATGCAGAGATCCTATCACGTGGATGCAGGTCATTTTGGGGGAGGGAGGAAGATCTGAATTATATACATGTGGTCAGTTCTGCTGA
GAGCTTCATCTCTTGGTTGGCAGAGGGTGGTGTTATCTGGCCACAGGCAAGGCCCCAATGTCTTCAGCCTGCTTGGTTCAGACATTATAAAACT
GTGGTGGCTTCCTTCCTTCTTGGTTTATGTTGTCTCTGAGGCCTCATGCCACCGTTGAGAACCATAGTGGAAATGTCATCAACACTGAACATGT
TATAGCCCTTTCTTGGTTCCACCAGTCCCTTCATCCCCTACCAATCCCTTCCCCTTCACCTCCATGGTCTTGGTGCTAAGATAACTTTAGAATC
ATTGCTGCTAGTCAATAGCTTTTCATTATATAAATATATTATATATATTATATATTATTTTTGAAATATTTTTGTTTGTTTTCAACAGTGATGT
AGCATGTTAAAAAACAAAAAACAAAAAAAAATGGGTTCCTCCAACTGTCCCACTGCCAGGCCTCATATGCTGCCTTCTTCAACAAATCAATGCA
CCCCTGCCTGGTGACATCCACCCCACCTCCCAACCCAGTTGCACACATGCTTCCCTACCCTCCTCTGAGCAAGAAGACAGTTAGCAGGAACTAG
CAAGGAAAGGCTGAAAGCCTCCTTCTGAGGCTTTGAGATTCCCAGCCCCATTCCACTTCCCCACTTTAAACTGATGTCTCCCTCCATCTGCTCC
TCCCATCAGGGTCAAAACCTAACTGTGGTCAAATGGGATGCTGTTGCAAAGCACCAGGTCCCACCTGGCCCAGCCCCAGCCTTGGGACTTCCTC
TCCCCTCACACACGCAAACTGCTGTGTCTGGGGAGTTTTCACTGAACATTGCAGAGACTAAGAAGGGTTGAGGGCAGAGATGGCTAGCAAGACA
GGGCTTGGTGAGGGCAGAAACATGAGGTTGAGATCCTTTCTTCTAGCCAACAGTTGCCTTACACCTTACATTGGGTAATGGGTAGGGAGGAGCA
GGCTAAGGCTCCCGCTCATTTGAAAACCAGGAAGAGAGAACCAGTGTCTTCCTGAGCACCTGGTCAGTTGGAGCTACTCTTTTTCCTCTCAAGA
GATCATGGCCAAAATGAGCTAAAATCTTCAGCTAGAAGGGGAAAAGCTTATGGGCCAGTGCCAGTGCCTACCCTGTAGTTCCTGAGAAAGCTGA
GAGCAGGTGACCCACTTCTGGCCTAGCAGAATGAGCTGCTATGCACAGCATGCAGCTGCAGGGGTCACTTCCTGAGCTGGCCACCAGACCTCGG
AAAAGCTAACCTCTCAGGTGGTCTTGAAGTTAGGTTAGGGCATCCCTAAAACTCTGGGTGCTTGTGGCTTCTGCTGAATTTAGCCATGCCAGGG
CTGTGGCAGACACTCTGTAGGCCACACTGCCATGGGACAGGGAATAATTTGGGTGATACACCACTGCAATTTACACGGGGTCTCTTCTCAGCTT
GGATGCTCCCTGGGAGGCCCAGTGCTTCTGTCCTGTGAATTCTGCATGTGATTACCCATGATTTCTGTCATAGGCATTTCCCACTCTTCTGCTT
GCTTGAGAAGGACTTGGACTGATGGGACACTCAGGGTCTAGCCCAGGGAGCATATGCTTGGCTAACCTAATCTCCCCTTGATGTGTATACAGAA
CTGTGGAAAAGCAGTTGGTGGATCCCAAATGTTGTTACTTCAGACAAGACAGAGCCCTTTAACTCAGCCTCTGGCTTAGGAGTGATGACTACAG
GCTTAGAATGAAGTGTGTCTCTGGGGCTGAGACTTGGCATAACTGGGCTGGCTGATTAGTGACTTTCTTTGGCTCGTAGGGTTGGTGGGAAGTG
AGATCAACCTTGAGGCCCAGGTTTGTGGCCAACTGTGCCAAGGTGATACCTGGCAGAGCCTGGGAGCCAGAGCCCTTATGATTAATATGATCTG
CTTTTCCTTCATGGAGGACAAAGAAAAAATCCACTGCCATCTAGTATCTGTGAAACATGAGGACAGCGCAGTCAAGTCTGTAACTCTTGCCATG
TCAGAATCCCCAAGTTTTGCCTGCCTGGTTGAATATGAGAGTCCAGGCATCAGAAACAGCAGCCTTATTTAATTTAATTTTTCTAATGACTGGC
CTATGACACCTTGTGATGCTAGGCACATCCTCATTTCCCCATCCTCACTTGGGACTGAGAGCAGGCTCAAGTTCCAGGGTCCCTGGATGGCAAG
GTTCAGTGCTGGGCCCTGGAATCTATGGCACTTGGGGGTCTCTGACCTCAGCCTCTGCCACATGTTTCCAAGTTGAGTTGTTTTGCTGAGGTGG
TCCTCCCCTTGAGTTGCTTATGCCAACCCTTTAATTTAGGAAAAGTACCTTGTAATTACTTAAGGTAATGTTTAAATGTTTTCCATTCATTTGG
AGGTGGTGCCAACAGGGGGGAAAGCATGCAGAAGGCTGGAAACAATAGCTGAGACCCTACTGTGGGCCCACAGCCCTGGCCCAGCCGGCACTGA
GGGGCTGGTGCCATGTTACTTGATCACCTGGAGCCTGATGGGACCCAGGAGGTGGCCTGAGGGGATGAAGTATAACTTCCCTCTTCTGGATCCC
CCCTTTCATTTTCCTTCTACCCCCTCTAGGAATGGGAGTTCTAGACCTAGGTCTGCTTTTGGCTTTCAGTCTGGGTTGATTTCCAAGTCGAATT
CATGCTTTTTCTTGGCCCATAGGTCCAATTTTGGCAGAAGGCATTGGACTTGTGCCCCCTCCCTGCTTCAAGAGGCAGATCTAGCCAGGCCAGG
CTGAAACAGCTGAAACAGGCAGGCCAGTCCTCCTCAGACAAGAAAGGGGTTTTCAGAGGGCAGTGGTGTGCCCATTCCAGACACCAGTCTTCTG
GGGAGGTGGTGCCGTGTCATGGGTTCTAGTCTGGCCTCTTCCTCAGTCCTCAGGAGGACCCAAGAGACTGGCACGGCCCTTCTCCTGCTTGGAG
GGAAACCCATCTCCCACTTGGTGGGGGCCCTTCTCTTGCCATCTGTTGGTTAGGGTGCTTGAGCTGAGTGGATGGTGCTGTGATATTTTTAAGG
AGCCTTTCAGGCAATGTTTGCATGTTAGCCAGAGGGAGAAAAAGTGCCCTTTTGGGAGGAAAATGGTACGCCCCTCCACTTCCATCTGGCACTC
CCTTCCCCCCCACCCCCTCAAGTGGTATCACCCTGGAAATACCTATGCAATCCAGTCTCCCTAGGAGAGAGTGCATGGAAGCAGGGGTATGTGC
AGTGTAGAATACAGATGCTACAGCATATATGTTGTATATATGGACATATACAGTACGTATACACACAGAGTAAGAGAGTAAATCACGTCTATAT
ATCTATAAATAATATCCTATATATTTATACATTTCTATGTTTTAAATAGATATAAAAATAGAGTCTATAGAGCTGGGAGAGCAGTGGGAAGCCT
GGCGCTGTGCTGTGCAAAGGGGAAGGGAGCACGGCCTTCGGAGAGGGAGCCGGGGAAGGCCAGCAGGCAGGGTTGGCTGGCAAGGGGGCCTCCT
ACCCGGAGTGTTGGAGAGGAGAGTGGCTGGGTCCCGGCTCGCTCCATGCACTTTCTCTCCTTTTCCACAGGCTTGGTCTGAGGTTGGAAGGAGA
TACCCCCTGAGCTCCAGCTGAGGTGCCCCCTACCTCTCCCCACCCCCACAGCCCACGCTTAGGCGGTCACTGCTGCTTGGCAGTAGGACGTGGT
CTCTGACTCCTGGTGGAGGGACCACTGCACAAACTCCTTCAAAACCCTCCCCCACCAGGACTGAGCAGCGTCAGTGGCAATAGGAAAGGTCCAA
ACTGGATCAAGAGCTGGTCCAGGAAAGATACCGCCCCTGCCCTGTTAGATGCTTCTGCTGCCCCTAGAGGCCAAGCCCCTGAAGTGCAGCCGTC
CTGGCCTCCCTCACTTGCTCAACCACTGTCAGGAGGAGAGGATGTGGGCAGCATGAGCATCGCCAGGCAGCGCTGCCCACCATCCACAGGCTTT
CTGGCCAGGGCAGGGGGCATCAGCTAGCAGGAAACGGTGGGAGAGACATATCTGCACACTCATAAATTCAATGGCTACTCCAGTCCAGAAGCAG
GGCTTTGGCCCAGCCCGCTCCGCGCAGCAGCTGCTGCTGGCTGTACTCAGGACAACGCTGTTCCCCCTCCCTCACAGAGGCCCCGTGGCCCCTA
CCCCACTCCCGCCGTAACAGGCAGGTTTAGTTCACATACACTGTTCGCATTCTGTGACTTGAGGCAGAGGCTGAGCTGGGATACCCCAAGCTCA
TCCACTTTCGTTGGGGAGGGCCCCTCCCTGGGTTTATCACCAGCTCCTAGCCCGGGCCGGGCGGGGATGTCTGGGGGCCACGCGGGGGCACTGG
TGCAAGCTGGCAGCATGACAGAGGGCTTGTGCAGCCCTGACGGGACCCAGAAGCCCATTTGCTCGCAGTTTCCTCTCTCTGTTTTTTTCCTCCT
GGAGGTAGGAGAGAGGGCCTGACCAGGCACCAGATGATGGAGCAAGGGCAGCTGCATGCTCCCTCTCTCCAGACCAGCCTTCTGCTTTTGGGGT
CCGAAGGGGCATTTGCTCCCATCCTGAGCCTCCTCTGCCCCTGTCTTGCTCTTCCCTACCATCCTACAAGTACCTCAGTCTCCAGCAGGCCCAC
CCCTCCACCTGCAGCCCAGGGCGGGTCTGTTCTGCCAATGCCCACCTCCTTGAGCCACAGTTAGCTGCCAACTGGGTCTTGGGACACCCTCCAG
TACCTGGCTCAAGAGAGACCAGGCCGGGCCGAGCCTTCTTCCCACTGCAGTGGACTAGACCCACGGCCAGGGGATGGGCATCCCCAGGTAGCAA
TCCCACAATGCACTGTACCTCAGAGAGAGAGCACGCCAGGGGCACCAAGGGACCGAGCCCTCTGTCCAGAGGGGGACAGCGGTCACAATACTGC
TCACCAAAAGACAAAGGCCAGGCTGCCCTCGGGCACCTCTCAGTCTTCACTTTTGTCTCTCCGGAAGAACCAGCAGTCTGATTCCGTCTATTTC
AGCCCCCGTCTCTCTCTTCTCCACCCCCACGCTGCTGAACCATTTTCATGTCAATCACAAAGGAAAAATAAGTGGGGATGGGGGGAAATACCTA
GGAGTCTATTATCACATACATATTAATATGTTAATACTTTCTTTAAAAAAAACCTCTTGATGTTATTATTTTGCAGACTACGCTTTATAGTACC
TGTGTGACGGGACCTAGAACACTGGATACAAATAGAGCTATGTTGGTTTATCATAATATGTACGCAGAAACTTTCTTTTTGTCATATTATCCTT
GTAATGTAAGAAGATTGTTAATAAAAGCATTTAAATTTACTCACCACTGTTGAATTGCTGCCTCCTTGCCTCTATCCCCGGCCTGTGCTCTGGT
TGACTCAGAACGGGCCCCACCACAAGGGCTCAGGTGTCAGAATAGGAGCTTGCCTGCTGCCAGGTTGGAAGGAACTAGAAGAGGCCACTAGCCC
CTTCTTGCTGCTCTCATTGTTCCCCCATTAGAGTCTCTTTCCAGCAAGTAACCTACGTGCCTCGCCCACCAGGCAGGCCAAAAGAGGTGTGACC
GAAACCGTTTAAAATAAATCTCTCTGCCCCCATATGCATTCATCCCACAAGAATCTCCTGGAGGCAGCACACAAGGGCTGTT
HUMAN SEQUENCE - mRNA
GTCTTGAGGACCATCTCTCCCGGCAGCATACCGTGTGGCTTCACACTGCTCTGCCTCTCTGAACCTCGGTTTCTTCATCTATAAAATGGGAATA
AGAGTAAGCCACCTCAATGGACTGTGGGAGGCTTAAGTAAATTGAAGTGCCATGCAAGTAGCTAGCATGCAGTTGCAGCTCAATGAATATTATG
ATGGCCGCAGATACGATGGCTACAGCTGGGGCACCCATTTCGGGTCACAAGGTAGGGTTCAATGTTGAAGATGGCAGAGCCAATTGCATCCCTG
ATGATCGTGGAGTGCCGGGCCTGCCTGAGATGCTCACCTCTCTTCCTTTACCAGAGAGAGAAAGACAGAATGACCGAGAACATGAAGGAGTGCT
TGGCCCAGACCAATGCAGCCGTGGGGGATATGGTGACGGTGGTGAATCCGAGCCAGGAGTATGGCCAGCCCTGCTCTAGGAGACCGGACTCCTC
GGCCATGGAAGTTGAGCCCAAGAAACTGAAAGGGAAGCGCGACCTCATCGTGCCCAAAAGCTTCCAGCAAGTGGACTTCTGGTTTTGTGAGTCC
TGCCAGGAGTACTTCGTGGATGAATGCCCAAACCATGGCCCCCCGGTGTTTGTGTCTGACACACCGGTGCCCGTGGGCATCCCAGACCGGGCGG
CGCTCACCATCCCACAGGGCATGGAGGTGGTCAAGGACACTAGTGGAGAGAGTGACGTGCGATGTGTAAACGAGGTCATCCCCAAGGGCCACAT
CTTCGGCCCCTATGAGGGGCAGATCTCCACCCAGGACAAATCAGCTGGCTTCTTCTCCTGGCTGATTGTGGACAAGAACAACCGCTATAAGTCC
ATAGATGGCTCAGACGAGACCGAAGCCAACTGGATGAGGTACGTGGTCATCTCCCGGGAGGAGAGGGAGCAGAACCTGCTGGCGTTCCAGCACA
GTGAGCGCATCTACTTCCGGGCGTGCAGGGACATCCGGCCTGGGGAGTGGCTGCGGGTCTGGTACAGCGAGGACTACATGAAGCGCCTGCACAG
CATGTCCCAGGAAACCATTCACCGCAACCTGGCCAGAGGAGAGAAGAGGTTGCAGAGGGAGAAGTCTGAGCAGGTTCTGGATAACCCAGAAGAC
CTGAGGGGTCCCATTCATCTCTCTGTGCTGAGACAGGGCAAAAGTCCCTACAAGCGTGGCTTCGATGAGGGGGATGTACACCCCCAAGCTAAGA
AGAAGAAAATTGACCTGATTTTCAAGGATGTTCTGGAGGCCTCACTGGAATCTGCGAAGGTGGAAGCCCACCAGTTGGCCCTGAGCACCTCACT
GGTCATCAGGAAAGTCCCCAAATACCAGGATGACGCCTACAGTCAGTGTGCAACAACAATGACCCATGGTGTGCAGAATATAGGCCAGACCCAG
GGGGAGGGGGACTGGAAGGTCCCCCAGGGGGTCTCCAAGGAGCCAGGCCAATTGGAGGATGAAGAAGAGGAGCCTTCATCATTCAAGGCCGACA
GTCCTGCCGAGGCCTCCCTTGCATCTGACCCTCATGAACTTCCCACCACCTCTTTTTGCCCTAACTGTATTCGCCTAAAGAAGAAGGTTCGGGA
GCTCCAGGCAGAATTAGACATGCTTAAGTCTGGGAAACTTCCTGAGCCCCCCGTATTGCCACCACAGGTACTGGAGCTCCCAGAGTTCTCGGAC
CCTGCAGGTAAGTTGGTTTGGATGAGATTATTGTCGGAGGGCAGAGTACGCAGTGGGCTGTGTGGAGGGTAGCCTAAAGCTCTCTGTGGAAACC
ACCTTCCGGGAGACCTGAGGAGTGTAACGTGGAGGCGGCTACCTCCGTGGGTGGGAGCCCAGGTCCTCAGTGTCTCTGGCAGACCCATCGGCAG
CTCTGCCAGGTGCTCCATGTGTTGCCCTTGTATCCTCCTTGTCAATAAAGGAAGTTCCGCTGCAGAAGGGGTGTGTGCTGTGTTCTTGACCCGT
TGCCTTTCTCTGGTACTGGTGTCTTACCCCAAAGCCCAATTTCTAAACCCAGTCTTTCTCTGTCCCCAGTCTCAAGCAGGGTGTCCCACTGGAG
AGATCTCTTGGCTTCCCTAACTTAGTCCAGGAACACAGCCTTGTTCTTCTCTTCCTGAATCTCTGTCCTGCCACACATGGTCCCAGTTCCCTAG
CCTGGAGTTCTAGAAGGATGGAGAGTGAGGGGATCCAGGCCATTCA
HUMAN SEQUENCE - CODING
ATGTTGAAGATGGCAGAGCCAATTGCATCCCTGATGATCGTGGAGTGCCGGGCCTGCCTGAGATGCTCACCTCTCTTCCTTTACCAGAGAGAGA
AAGACAGAATGACCGAGAACATGAAGGAGTGCTTGGCCCAGACCAATGCAGCCGTGGGGGATATGGTGACGGTGGTGAATCCGAGCCAGGAGTA
TGGCCAGCCCTGCTCTAGGAGACCGGACTCCTCGGCCATGGAAGTTGAGCCCAAGAAACTGAAAGGGAAGCGCGACCTCATCGTGCCCAAAAGC
TTCCAGCAAGTGGACTTCTGGTTTTGTGAGTCCTGCCAGGAGTACTTCGTGGATGAATGCCCAAACCATGGCCCCCCGGTGTTTGTGTCTGACA
CACCGGTGCCCGTGGGCATCCCAGACCGGGCGGCGCTCACCATCCCACAGGGCATGGAGGTGGTCAAGGACACTAGTGGAGAGAGTGACGTGCG
ATGTGTAAACGAGGTCATCCCCAAGGGCCACATCTTCGGCCCCTATGAGGGGCAGATCTCCACCCAGGACAAATCAGCTGGCTTCTTCTCCTGG
CTGATTGTGGACAAGAACAACCGCTATAAGTCCATAGATGGCTCAGACGAGACCGAAGCCAACTGGATGAGGTACGTGGTCATCTCCCGGGAGG
AGAGGGAGCAGAACCTGCTGGCGTTCCAGCACAGTGAGCGCATCTACTTCCGGGCGTGCAGGGACATCCGGCCTGGGGAGTGGCTGCGGGTCTG
GTACAGCGAGGACTACATGAAGCGCCTGCACAGCATGTCCCAGGAAACCATTCACCGCAACCTGGCCAGAGGAGAGAAGAGGTTGCAGAGGGAG
AAGTCTGAGCAGGTTCTGGATAACCCAGAAGACCTGAGGGGTCCCATTCATCTCTCTGTGCTGAGACAGGGCAAAAGTCCCTACAAGCGTGGCT
TCGATGAGGGGGATGTACACCCCCAAGCTAAGAAGAAGAAAATTGACCTGATTTTCAAGGATGTTCTGGAGGCCTCACTGGAATCTGCGAAGGT
GGAAGCCCACCAGTTGGCCCTGAGCACCTCACTGGTCATCAGGAAAGTCCCCAAATACCAGGATGACGCCTACAGTCAGTGTGCAACAACAATG
ACCCATGGTGTGCAGAATATAGGCCAGACCCAGGGGGAGGGGGACTGGAAGGTCCCCCAGGGGGTCTCCAAGGAGCCAGGCCAATTGGAGGATG
AAGAAGAGGAGCCTTCATCATTCAAGGCCGACAGTCCTGCCGAGGCCTCCCTTGCATCTGACCCTCATGAACTTCCCACCACCTCTTTTTGCCC
TAACTGTATTCGCCTAAAGAAGAAGGTTCGGGAGCTCCAGGCAGAATTAGACATGCTTAAGTCTGGGAAACTTCCTGAGCCCCCCGTATTGCCA
CCACAGGTACTGGAGCTCCCAGAGTTCTCGGACCCTGCAGGTAAGTTGGTTTGGATGAGATTATTGTCGGAGGGCAGAGTACGCAGTGGGCTGT
GTGGAGGGTAG

TABLE 9

MOUSE NOMENCLATURE
ICSGNM N/A
Celera mCG17918
HUMAN NOMENCLATURE
HGNC N/A
Celera hCG23764
MOUSE SEQUENCE - GENOMIC
TTGAACTCCTGTCTCTATGTACCCAGCTGCTTGTGAATAAACTCTATTTTTCCGAAGCTTGTTATCTTAGGGTTTCAATTGATGTAACAAAATA
CCGTGACCAAAAGGCAAGCTAGAGCTAGAGAGGAAAAGGTTTATTTGGCTTAGACTTCCATACTGTAGTCTGTTATTGAAAGTCATTGAAGGAA
GCCAGGACAGGCACTCAAGCAGGGTAGGAACCCATGGAGGGGGCTGCTTACTGGCTTGCTCACCACCCCCCTCCTCTTTCTTTTCTTTTTTTCT
TTTTTTGGTGTTTTGAGACAGGGTTTCTCTGTGTCATCCTGGCTGTCCTGCAGTTTGCTCTGTAGACTAGACTGGCCTCAAACTCAGAGAGATC
TGCCTGTCTCTGCCTCCTGAGTGCTGGGATTAAAGGTGCGTGCCACCACAGCCCGACTTCTCAGCCTTTTTTCTTACAGAACCTAGGACCACCA
GCTGGGTGTGAGCACTCACAATGGGCTGGCCCCTCCTTGGGGCCTCACCCATCAATCACTAATTAAGAAAATGTTCTACAGGCTAGCCCACAGC
CTGATCTTAGGGAGGCATTTTTCTCAACCGAGGTTTCCTCCTCTCAGATGACTGCAGCTTGTGTCAAGTTGACAGAAATCTGCACAGCGCACTT
GTCGTCCCAGGGATGGATGCTTGGCGCAATGGTTTTGTGAAACAGGCCTGGTGGGGTTGCAGAAGACAGCTCTCCTAGCCTAGAGCTCCGGTCT
CTCACCTACTTTCCTTTGACCGCAGACCCTGTGACAGTGCTGGAGAGAGTGAATGTCCGTTCAGGCTTGGAGACGCTGGGTTAAGGAAAATAAG
CAGACCCTGAGCCCATGGACAGCACCCAGGTGCTTCAGAGGCTTCTGGAAGCTTCCACAGCTGCTTCCGGTTCCTTCTCCTTCCACCACCCCCC
TCCTGCCCTGGCTTCTTCTGGGACATTCCATCTTTCTTCAATAGCACATCAAAACCTGGTGCCCTCCTGGATACTCATTTTGCCAATGCGCTCA
GGCATCCTACTGGGACTGCACCTCGGGAAGTGAGGGGAGGACGGAAGGAAGTACTCCCCCTCTTCTTTGAACTATGCTTTTTGATATTGTTCAC
TGCTATGTCCTACATGGCAAAGATGGTTTCCCTCCAAGGGAAGGGCTTCCTGGGCTGTAGTCAAGATGCAAACAGTAACTACTGCCGGGTGTGC
TGCCATGACAGGTGTGCTGCCATGTGGGTGTGCTGCCATGCAGGTGTGCTGCCATGTGGGTGTGCTGCCATGTGGGTGTGCTGCCATGTGGGTG
TGCTGCCAGGGATGGAGACGGACTGGCCTCAGCTGGTGATCCTCCTTTGAGGTTTCACGTGAGCTCAGCCAGGTGGAGGTGCGTCCATTGCACG
TGAATGCGCACACATGACTAGTGAAAAGAGTACAGGCTCCATCGGCATTACTCTCTTTCTTTGGGGTCTTAGCACAAGACAGCTTCAGACTCCC
AGATGCATGAGGGTGGAAAGTGAAACATTCCATTTCTCCACTCCACACGAGTGTGGCCCCCTTTTCTGACAATGAGTTTGATTGGCTGGGTTTA
GAGAGTTTCCATTTCTCTCTGCTCTGAATCAAATTGTGAGGACTCTTTTGCTTGAATCCAAAAATGAAATGAAGGGCATCCCGCACAAATAACT
TTCCTTAATTGTTGTTTTTTTTTCCTAGATGACAGCTGATGGAGAAACATCGCAGTATGAAAATACATATGGTACTTGTACACACACACACACA
CACACACACACACAGAGACACACATACACAGACACACACACAGACAGACAGACAGACACAGACACACACACACACACAGACACACCACACCTCT
GTTGTATATAGCCACTCACATTCTTTCCCCCTGCTGAAAATCACTTGACTTCCTGCAGAAGAACTTTCTCTCACATCTGCTTCTAGTGGCCACT
GTATCCAGGGTGGTTGAGCCCTACTTTGTAAGCCAAGAGCCTGAAACATTTCTTTTCTGAAGTCAGTCCTTCAGATTCCATAGGGCAAAGGACA
AACTAAAAGCATGATGTAGGCATTTCTTTTTTCTTTGTTTGTTTGCTTTTCGAGACAGGGTTTCTCTGTGTAGCCCTGCCTGTCCTGGAACTCA
CTTTGTAGACCAGGCTGGCCTCGAACTCAGAAATCCGCCTGCCTCTGCCTCCCAAGTGCTGGGATTAAAGGCGTGTGCCACCACACCCCGTGAT
GTAGGCATTTCTATAATTATTTGTAACATAACCACTTAAAAGCAAAACCATTTTGTTGTTGTCATTCTCTCTCTCTCTCTCTCTCTCTCTCTCT
CTCTCTGTGTGTGTGTCTGTCTGTGTGTGTGTGTGTGCACATGTTTGTGTGTATGTGTGCCATGCATGCACATGCCACAGCCTGCTTGTGGCAG
TCAGAGGAATATTTTCAGGAATTGATTTTCTCCCTCCCTCTTGGGCTCCATCCCGGGCTCGCACGGCAAACACTTTCACCACTGGCCCATCTCC
TCGGCCCCATAAGAACGATTTCTAATGTGTGATCACAGTCTGGCCAGACTTGGAGCCTATACTTGTCTTTTGTTGTTTCTTGCCATCAACCAAG
ACAGTCCCTGAGGCATCTTCTGGTAGATCCTGCTTCTTACCATAAGTGACAGAGGTTTATGGGGTGGGGAACAACTTTGTCTACATTTTTAAAA
TGACCACGCGTGCAATGGGGTAGAAGGGTGGAGTTAAAGTTCGTCTCTCTTATACCACGCCCAAGCTTGTGTGGGTCATGCTTACTTTCCAAGC
TTGTTTCTAATCCTCCTTCACAGCCTCTCTATTCATTCAGCACAGGTCTTTTCCCCTGAAGGTAATTAGAGCTGGTTTCTTCCGCTGCCTCCCA
GAGTGACTTGACTGATGACTGATGATGTACATCATAGAAGATCCACGTGTCCATCCCAGTGGCTCATTTGAGTGTGCTTTCAGGGACACACAAC
CCCAGCCCATCTCAGAATCCTGGATTTCTACACCTCCTTTCCATGGAACAGTTGTGGTGGTCCCAGGTCCTGAGACCCTCTCCAGTTTGGCCTG
GGAATGCTCAAGTGTTTGAGTGGGCCCTCCTTACTGGCACCCTTCTTCTTGGGGTAGAATGACATCAGTGCAGTACTCACCATGGGCTCAGCCT
GACTGAGTAATAAGAGGATCTGTTTGTGCAATTGTCTCTGCAATGTGCAACACAGAATATGGCAGAGACTCAAGTCAACTTTGTTTGGAGGAAA
GGGACAGCATTGGCTCCTGTCACTGAAGAAGGCACGAGTGGGAGTGAGCAGGGACACGGCTCCAGCAGGGACAGCTGCTCCAGCTTCTGTCTCA
GTTTAGCTCAGGTGACATGCTGCTACCCAATGCCTGGAGCTGTCAACCTCTACCTGCTCCCAGTGCAGGATCTTCATTGTCATTGGGTTATTGG
TCCATCTCTGGGATGAGAGCCTGCCTCTGAGGTCCGCTCTCAGGGACACCCGTCTCAAATACGGTTCCCGAGGAGCAGAACTGAAGCGTGTTTA
TTCAGGATCTTGCAGTTGGAACATTCTTTGTTGACTAAAAAGGACATAGACGGAAATAAAAATGGCATTAATTCTATGCCTTCTTCATTGGGTC
TAGGTGTCATTCATACAGCTCTCCATCAGGAAGCTCCTAGCTGCTGGGTTAAAGGAAATATGTGTGAGCTCTAGGAGGTCAAGCAGTGACTTTA
AGATGTCTGTCTGCATAGTCCCTGTGAGTTGGGCCAGCAGGGTTCATCTAACTGTCCTCCTGGGGAAGTGTTCTGAGATCCTAGGAACAGGTGA
AGTACCAGAAATGGTTTCAAGTGGGACTGGGCTAGCTCACTTTGGGGATGAGCACTGAACGTACATTCTAAACAGACGCCCAGGTAACACAGTA
AGAGCTCACTGTGTAGGCTTGCAGGTATCTGCAGAGATCCTGAGATGTGGGTCTGTAAACCTGACTGAGGCGGTGAGGGCGGTGAGGGAATGGA
CAGAGAAATGAGAGACACGTGGATATAGAAAAGCTAGGGTCAGGTGGGTCGTGTGCACCCTGGTGGAGCGGCACCAGCTTCTGTAACAATGGGG
AACCTCAGTCATTTATTATGTATGGCAGGGTTAGCTAGTATTAGGTGGAGCTCAGTCTCAGCCTGTGAACAGGAGGAGGAAGTCATAGTTGCTG
TGTTCATGCACACTGGCCAATGGCTAGTGGATACGAGTGAAAGGACAGAAGGAGCCTTTGCCTTTCCAAACCTGACTCAGGGAAAAGGGTTGGC
TACTACTTCCCCCATGACATTGCTGTCTCATGTAAAATCATGCATGTTCATTCAGAGCTCTCCTCCCTCCCTACAGGTGTCCGTGGGAAGAAAC
AGAGAGACCTAGGGGGAAGACAGACTAGCCAGAGAAGAGAGGAGCCGGCACAGGACGCTGCTGGAGTTCTCTCAGGAGGGCCTGTTGGAGGACT
GATCTCACCCCGCTGACCGGTTGCTCTGTGGATCTTTCTGGAAGTCAGTTGCTAAATGTGGGCTGCTAGGTGGAGGTGGGAGAACGAAGAGAGC
GACCTTCCAGATAGTCCCTGTCTGTTGAGCGTGACTTTACTGTACCTGGTGGGATCCCAACACGAGGATGGGTGGGTCACATTAAAGAAGGTCT
TACCGTGGGCTCAGTGAAGAGATGGCTCAGTGGTTGGGAGTACTTGCCGCTCTTGCAGAGGACGTGGGTTCAATTCCTGGCACTCACAAGGTGG
CTTACAACTGTCTGTAACTCCAGTTCCAGGGGATCCAAGGCACTTTTCTGGCTTTCAGGAACGACAGGTCCACACACATGTATGCAGGCAAACA
CTTAGATACATAAAATCATTTTTAAAACGTCTCACCAGGCCAAGTGTGGAGGCCAAAGTCCTTAATCCCCATACTCAAGAGGCAGAAGCAGGAG
GATCTCTGTGAACTTGAGGCCAGCCTGATCTACCTAGAGCGTTCAGGCCAGCCAGGGCTACATTATGTCTATCCAAAACAAAACAAAACCACTC
TAGAACTAGGTGTGTGGAGATGGCCACGGGAGGAATGGAAGCCGAGCCCCAGTTGCTTTGAGACTGTACTTTTCGCCTTCTCCCATTAGACTTA
TTCTTCTTGTCTGTGTTGGGGGTGGCGTTCAGCCTTGCTTAGCTTTAGGTGGCTTTGCCCATCTCACAGTTAGGCCCTCTATTCCAGAAGAAAG
CTGCCTGTCTCAGCAGGCATGAGCTGGGCCAAAGCTGCCACGTGGGCTCGGGTAAGCCACAGGCCTGTGTATCTCTGACAACAGGGAGTGAAGC
TAAAAATGGCGAGTCTTGTCTTTCTAGAGAAGTCTGACTCCCACTCTGTTCACTTCTTGCCTTGAAGGCAACGGTGCGCAGAAGTTTTGCTTTT
AACCACAATCCACAAGAACTGACCCAGCAAGCACACATATACCCCCATTCTCACCTTGATAGCTGTCTGACATCCTTTCCCTGGAGAGGACCTT
CCTATTATTTTCTGTCTTGTTTATTTGTTTGGTTTAAAGGGTCTGGTTATGCCTCTACACATGAATTTCTCTGCCCATTGTAAAGTCACTGCTG
TAAGGCAACATGGAGTGGGGGTTGCCCTTCTTATGCCCAGGAAGCTCTCCTAGCATCTCAGGCTGCTTGAGGACAAGGCTGCAAGATGTTGGCA
GAAGACAGTGGCTGCAGCTTAGCAGCCACCAGGCTCTCGTTTTTAAAATGTGAATCTTTCTCTTCTCACCGCCATCTCCCATTCAAACAAGAAA
CTGTGGCCTCCTTATCAGTTCCTGCCTTCCTTGGAGGTAGTACTTCTCAGTGCCCAGGCCCCATAGGCAGCCCTTCAGCCTAGCTGGCTCTGGT
GCACACCCACCCAATCCACTCCAGGTGTCTCTAATCTTCCCTTTCCACACAGTCAGTATTGGGGAGCCTGCTAAGCTGACGTTTGTAGTCCTTG
CTGAGGGACTGCTAAGGGGGCCATGTTTGCTTTTCTTGCTCAATGGTCAGGCTGTGTGAAGGAAGAGCACTGTTGGCTACTGGTCCTGACAATC
TGATCTACTTCCCAAAAACTTAAACTGGACACACAAAAATAGTCTGTGCTGGGTGTTAGAATTGAGGCTTGAGGATCGAACCTATATAAAATGC
AGTCACATGGTAAACTCAAGCTTGGCTGGGAACAGAGCAGGGAAGCAGCAGGGCAGAAGCCAGGTGGCCATAGACAGCGCTGCATAGCCTGCAA
GGAGAGCAGTTGTTAAGGCATTTATGGACTATGCCTGTGTCTAGATGCTGTGGACAGAACCTGATTTGACACGGACAGGCTCCTGTTCCTAGTC
TGTCAGAGGCTCAGCAAAGGCTATTAGCCTGAGTCACAGATGGCCTAAGGACTCAGCTCCCCTTCAGGACTTGGTCCTGTTGAGGGTCCTATAT
TTAGCCAGTCATCACAAAACTTTGTCCCACAAGCCATCAACAAGAGATATCGCTCCTCACCAAGAGGGACAGATGCCCAGAGCTATTCACAGTC
ACATGGGTTTCAGGATCTCATCTGGGGACACCAGGGCAGAATTCTTCCACATCAACACCCAGACCACTCCATTGTTTCTGTGTGTGCACAGACC
AGCAGGATAGGGTGGTGGTTTAAAAACCAAAGCCTGGAGAGAGCGCTCAGAAGTTAAGAGCACTGTCTCCTCTTCCAGAGGTCCTCAGTTCAAG
TCCCAGCAACCACATGGTGGCTCACAAACATCTATAATGAGATCTGGTGCCCTCTTCTGGCCTTAAGGCATATGTGCAGGCAGAATGCTGTAAA
CTTAATAAATCTTAAAACAAAACAAAACACCAAAGGCCATTCCCTCAGACCAGTCCTTAACACCCATAAAACAGCTAACTCACCAGAACCTTCA
TTTTTTGTCCATTTATTTAGAAAAAAATTAACATGAGCAAATGAGATACCTCAGTGTTACAACAGAGTATAGAAATGTCTAGCAATAATCAAAT
AATTTGATCTTTAAATACAAAATAACCACATGAACACCTAATATACAGGTTTCATCTGAATACATATTTATTAGATAAATATTAGAGGTCACAT
CATCTAACTGCATACAGCTTTGCAAGACTAGAAATCACAATTAGTTTTTTTTTTCTGACCAGTCAAAAGTATGAAATGATTGCAGTGTACATAC
GATGTACAAAGACAAGGGCGGGTTCTGTGGACGTCACTTCAGGCTGCACGTGTGGGTGTGGATGTGTGTACGTGTGAATCACCTGTGATCATGA
TATCAAAAACTTATACAAAGTATATGAATTTGGTTACAATTTTCTTCTGAAATCCCCGTTTCTCTTCATTGTTTCCATAGCACCCTAAAAATAC
ACAGGTGGCAGGGCCAGGACACAGAAGGTAAATAGTACATGTAGGTAAAAATAAAAACAAAAGGGAACAAAAACGCCTCTGCACACAGGGTCAG
TATATTACAGGAGACAAGGACGGAGTCACGAAGGCTAACAAACGGGATCTAGTATTCCACGTAGAATGAAGGAGTTCCAAGCCTTTTGTTGTTT
CTCTGTTTTGTAAAATAAAAACAATACACATTCCGGGAGAAATGAATGTATCTTGTTGACATGTCTATTTCTCATTTACATATGTACACACGGC
CCTTGAGTCGCTGCTGCTCTCTGCCTCGTCTGGATTGGTCAGGCCCGAGGGCCCATGGGGACCAGACCTGTAGCTCTCTGGGATTTAGGGCTTC
CGTTAGGGAGAAAGTGTTAGGAATCTTTTAAAAAATAAAATGGCTACAGGATACGTGAGACATGAATAAAGCTTCAAACCAAGAAGATGAGGGT
GATCGCCTGTGCGGGGGGCGGGGCCTTTCCCATCTGCGCATGCTCTCTCCCAGCCCAGCCGCTTAGCTGAGTGGCGGCTGGTACGTGCCTATGG
GTGGGTTGCTCGTTGTGGTAAAGTGCCTTGCTGAGACCTCATTTCGGAGGTTACTATGGCTCCAAGTTGTTGTAAGAAAGGACTGAGGATCTTT
CCAGAGCCTAGGCCTGCCTCTGTTTATGGATGTCACCTTTACCTGCGTCTGTCACTACCAAGGCATGGCTCCAGCCCCCGATGTCTTTGTAGCT
CTCTCAAACCCTGGATCGGCTCCAACATTTCTCTGGAAGGAGACATTTCCGGAGTGTGGGCTTCAGGCTCTGTGGTGAACTTGCTGGTGGGCAC
TTGCCTGGGAGGGAGCCTTAGGAAATCAGCCTCAGGTGTAGCCTCGGGTGTGAACGGAGAGATGACCAGAGAGGCTGGGCTACTGGGAAGTGTC
TAGGCCTTTTGCACTGGAAAGCTGTGGAGAACCTGGGCATGCCTTGACAGGAACTAGCAGGGACCCCAGGCTGTTCTGGGGCACAGTATCACGG
CAAGAAGCAGGAACTTGTGAGAAAGGCATTTGTCAGTCTGAAGCTGAACAATCCGTACAGTCCGACAGAGCACGGCCCTGTGCCGGGCCACCGA
CACTGGGTAGGGATAGCCACGGGGTACCTGGTGTAATGTCCCTACCAGCTGACTGAGAGTGGTTCTCAACAGTGGGTTGCCTGGTCTCTCAACT
CGAGCACAGCCGCCTCTTCTGCTCCTCTCTCATCTGTACAAATAGCCCTGGTTGGTTTTTGTTTGTTTTTGTTGTTTGTTTTTGTTTTTTTAAA
AAAGGACTCGGGTTTTGTCACTGAGTCAAGATATTTTAAAAACACCAGCAAAGGCTATAACCAGGTCATGAAAATGTTGAATATTATCAAAGAC
AATACTAAAATATTCACAAAGATATTTACAAAAGCGATTCTTAAAATAATTGATTTGCATACTGAACACACTGCTTGAAAAAACCTTAACAACA
GGACAAAGGAAGAGGAGGGGTGGGGAGGTGGCAGCTTTCCTTGTGCCTTTTTTTTTTTAGGGTGACAATAAGTCAACAGCTAGGGGTCACCTTT
CTGGTTCAATCTATATTAAGAGTGCCAGGGAGCACAGAGGGAGGCTAATGACCGATCACAAATGAGGACGCTGGCAGTGGACAGGAGGCAGGAG
AAGGCCTTGGGTGGCAGCATCGGAGAAGGGAGCAAAGAATTTACAGATGGGACTCATGTGAGCGGACAACCTGGCATCACCACAGGCATCACCG
TAGGCCATGAGGCTCAGGGGGACACAGACCAGCCTGGCACAAAGCAGGCGAGTGACAAGCCAGCAGGGGACTCTAAGCTGGATGGAGTGAAAAA
GCAGAGGAGGGCCAGAGGCTGCACTTGCTGGGTGAGCAGGAGTGTGGAGCGAGCTCTGGATGGAGGGAGAAATACAGGGAGTGACAGGCCATAA
ATGCCACAAAATGCCTGCCTGCTGAGATGCTCTGGTGCACCCAGCAAAGGTCTGGGCTGGAGACAGGCAGCTTTGAGCAGAGAACCTGCTGGAC
CACACTGAGAGACTTGGAGATCCATGAAGGCCCTGCTGTGGAGTCGTTTCCCTGGGGGCCTATGCTGCTTCCCTACCATGTGCCCCTACAGCTG
TCTGGGGAAAGGAGAACCCAGAGGGAAGTGGGTCCCATGGAATAGTCTCAGAGAAGGAAGTATGGGTAGGAGGAAGGAAAGAGAACATGGGGGG
AAATGGCTTGCTTGCTGTCCAGATACAATGTTCCTTTCCTTCAGACCTCCTTCTTCAGAGCTCCCCGCTGCGGGTCATTTAAAAGCAGGGATTG
GCTTGGGGCAAAGCTGCAAGGCCCTGGCTGCTTCTGTGGGGCTCTGGCTGGCATCGTCCTCTGGCCTCTAACTAACGGAGGGCTCCCTTTGGTG
TGGTGCACACACTCTCTGCTCCCCGCAAGGCATGGCCCTGGTCTGAGTTAAGGAGCCCTCCTCTGCATTGTCTTGGCTCTGGGGAAGGCCCTGA
CCTGTTTCCTGCCAGTCCCTACAAGGTGTGGCACTCTCTCTCAGACACGGCACTTTGTCCTTGTTCTGCTGAGAGAGTGTTTACAGGGCATCTT
GGCTTAGAAGTCAGAGGCCAAAGCCACGACCCCCGGCCTTGAGGGATGGTCTCCTCCCCAGAGGGTTGTGCTTTTCTGTGGTGTGTGTGTGTGT
GTGTGCGTGTCCTTCCTCACTGCTGCAAACTGTCACCACTCCTCCAAGGCTTGTCCACTACCCTGGCCTGGGGGCCGGAGCACAGGCTTCTTTC
GGGTTCCAGTCATCTGAAGGAGGCTCTGGGCCAAGAGGCGGGGATAAGGTTTAAAAACATGGAAGAACTTTGGGACACTGGAAGGTCGGTTGTC
TGGAGTCAGCACACATGGAGGGACCTACAGTCGCCATTGGCATGTCTCTTGCTGGGCCTGGATTCCCTCAGCTTGGGTTTGTCTTCCAGGGGCC
GTCTTCAGGCTTGCTCTAGGCATAGATCCCAGAGCATTCAGGCAAACAGGACTGGAGACCCACATGGCTTTCCCTGCCTAGTGTGCTCTGCTCA
GCTCTCAGCCTGAGGTCCTGGTGTGGGAGGCCTGAGCATGGGCTCCTGGGAGGAATAGGATGTGGGACAGATGGGGTAGGAGGCAGAGTGGGGA
GGGATGGCCCCAGTGGAGAGTGGCCTTCAGTTGTTCTCAAAGAGAGACAGGAGGTCATCGTTGCTATTGCTTGGAAGGTCGGGGGGGTCCAGGT
AGGAAGAAGCTCATCCGGGTTTGTGAGTTCCGGCAGTAGCTAGGAAAAGAAGAAGCCTCGTTGAGGTGTCTGAGGCTGCTCCAGGAGGAGTGAG
GGCTCTCTCTGGCCTTCTCACTCCCTTCCGCTTGCCTCCTGGAGTTCTACCTCAAAGACAGAAAACTTAACAGTGTCAAGTATGGGCCTGCCGG
CTGTCCCCAAGACCACTGCATCTCAGCTGGGGGCAGGCATACCCACACTCAACAAATGCCAAGGCCGGGGAGACCCACTGGGCTGACCCCTCCT
CTCAATGTCAGCCCCATGGCAGATCAAGGGCTGGATTCCTTTTGATTGCTCCTCTGTCCTCTCAGACCCTGCCAAGAGCATCTTTGGGTCACAC
AGCCTGCTGGTATAAGCCCTCGGTTTTCATCCATGACCACAAGGCAGGCCGTGGGAAGACCAGATGGTCCCCACAGTGCCCGAGACTACTCTCC
TGTAATCTTAAGTGAACTAGTGGCTGGCCATACTGTCCTGTCCCTGTGCTCCCCTGTCCCCACTCCCCGTGTGACACCCTTGAAGTATGTATGT
CACACAGGGGCAGAGGAGCTCCTGAATCTAGAAGCATCCCTCAGTCTGGCTAAGGAAGGAGAGTTGGGTGAGAAACCAAGACATCTGCAAACTT
TAGACAGCACTGCAGTTCTCCCTGCCAAGAGCTCTTTGATGCCACCGTGGTGGGTGTTGATAGCAGGGCCAGGAGTTCAGTACCTAGGGATGGG
GGCTGGCTACAGCTTCCAGCCCCTGGCGGGGGTAGGAGAGGAAAGTGTCCAGTCCCAATCTTTCTGTGATGCCCACCACCCCGCCTTCTATACT
CCTGAGGTCTTGGAATGGGAGATGCTTGAGTCTCAGCTGCCTAGTTTGACCCTGTCTCCCTCGAGTCATGTCCTCCCCACATCATCTTCTGTCA
TCCGACATGGAGACTTCTTGGAGTGAGCAATGGAGCAAGCGGAAGCAGGGAATGGAGACACACGGACAGGGAAACCGCAAGCAGACAAGACCAG
CAAGACGATGGTGGGTGGGGTGTCATTCTTAGCTCTTCCCCTGCCCAGGCCAGAAGGTAGATACAGAAATAGCAAGCTTGACATGGGGGTGTGA
GGGAGGGGATGGGAAGAAGGGCAAGGACCCTCTTGCACTATCTCCAAGGCTCTGACTGGAGGGATTGGTCAAGGGCTTACGTAGAAGGTGGGTA
GGAATGAGGAACAGGGCTATCGGGCCAAAGGGCTGATGGGTAGGGGTGGGGCTTGCATGACACACCCCCATATGGGGCTTCCTAGTGCTAGAGT
CAGGGGAGACTGGCTGGGGCCCATCCACCACCCACCCACCTTTCCACCCGGGGCCTTGAGCCCTTAAGTACCCCCCTTCTTTTGGGAAGGTCAC
GGTGCCGAGCCTGGCCCCAGACCAAGGTGTGTGGTGACCCCAACTTACATCCAGTGAAGGTTCCGGCATGTCGGATGCTCCCTGTGCTCCGGCC
TGACCCTCTAAGGCTGAGGAGGGGTTAAAGGTCAGGTCGCTGTGTGGATGCTTGCCGGGAGCGGCCTGTGCTGGCTGCCGGCGAGGCTGGGAAG
GAGGAGGAGCCCCACTGTGATGTAATGGAGGCCCTGCCTGGCTGCTGGGGTGTGGTACGTGCAAACCTTGTTGTATGGAGGGATGGGGCTGGTC
AGAACTGCCAGCGTGGGGCATCTGTGGAGGAGGAGAGAGCAGGAGAAAAGAGAGGTGAGGTGACGGAGGCACAACGACGGGATGCCTGGAGAAA
ATGAAAACCGAACAAACCAGGAAGTAACGGACCCCAAAACGAGGGCACCCCTCAACAGAGAACAATGATCCGCCGTTGAGGGACGGGGTAGAAA
AAAAACAAACAAATCGTCCTCTCCAGAAACCAACAACCAGGAAGGCTGTGAAGGGACAAGTGGAGAAGGCCTTCACAAAGCCTAGGAAGGGCCT
CGAGCCAGCACAGGCCAGAGAGCCTCCCACCCCCACACTGTCACAACAAACAACAGCGAGGCCAGAATGGACAAGCAAATGGACGTGTTTCTGG
GAAGCGGTAGCTGCTGGCAGCGTGGTGCAGGGCCCTCCCCCGAGGACCAGGGCAGTGCAGAGCAGGCGTGGACACAGCACGGGCAGGCGTGCAG
CTCCCCCTGGCTGGCAGTAAAGAGGAGACACGTGCTCAGCCCGCTTCCCTCTGGGATGAAACTCATCTGCAGTCAAAATGGCGGACCAGGGAGA
AAACCTGGCTGGCCAGGGGCATTTCAGGGTACTGCCTTCTCCTTGATTAGAAATGGGCTTCATGGTGGGGGTGGCAAGGAACATGGAGGGACAA
CTCTAAAGGTTAGCTAGGGAATTCCATCCTTTTAACTTACTCCATGCTGTTGTGGGTAGGTGGGTGTAAGGGGATCTTATGTAAGTATGTGCCT
GGTGAATGCTGTTTGCTGGTCTCAGCAGACCCTTCAAAGCAGAAAGCCTGAAGCGGCTCTGTACATGTCCAGCAAAGGCCTCAGCCTGCTCTAT
ACATTTGCACAGGGCCACCAGCACCCTCTTACATACTGCCAAGAAACTATCATTTTGGTGACAGAATGCTTCCTTGGTCTCGGATGATTTCCAT
CTCTGCCCCATCTCCATTTCTGGGTTCTCCCATGCCGGGTTGGCGAGTAGCGATCACGCCAGCCTTGATCTCCCAGACCCTAGTTCCTCTTTCC
ATTTCTGCTAAAAGCTGTTCCCCAAGTCTGCTTTGATCTGGTCTCTACCAAAAGTCCTGGTGTGCCCTTTCTTTGCAGGGTGCAGGGCTCCAGT
GTGACACCAGTATTACAGTATTCTTCCAGTGACCCTCATGTGGCATGGTATAAAGTGGCTGGAAGACAAGGCCACATAGTGGTAAGAGGCCAGC
TGAGGGTACCCACTTAGCTCACAACAGCAGATGTCCCATGACACCCACCCAGAGGAAACAGGGAGGGACTCCACACTCTCATGACCAGCTTAGC
TCAGGACAGACATCTCGGATCCTATGATATAGCTTCTCCCCTAGACCTCAACAAGAGTGTCTCAGAGCCAGAAGTACTGCTGTTGTGTCTGCCA
ATCTGCTGCCTAGTGCGGGGAGAACTCTTGGGCAAACCAGCGCTTGGTTAGAACCTTCTAGAGAATCCTGGACTACACAAAAAACCCTCCAGCC
ATCTGCCCAGCCAGGGCCAGCTGCTTCTAGATACAACAAGGTAGATACACCAAGAATAGACCTGGGGTTGTCTCCCCATGTGATACAATGCGTA
GCTAGGCCTGGTTGCTTTCAGTGAGATGAAAGCTGAGAAAGTAGGCACGCCTTTGTGGAATTCTGGGGGGGAGATATGAGCTTGATTCAGGTAG
AAGATGAACCAGGAAGCAAATAGCTTGGCTGGTAAGAGAGCAAATGGCTTTCTCTATGTGGCATCTTTCAGGATTTGTGAGTATGAGGACTTGG
GGTATACAGTGTGTGAGGGGTGATGTCGGTGACATCAGCTGTGTTAGGCCGAGGTCCTGGTGAGCTTAGAGGAGGAGAGCAGTGGCTCCAAGTT
GAGAACTTCAAAGTCAGAGTCTATATGTACTTTTCTTTCTCTTCCTGTCATGTTTTCTTTGAGGTAGGTTCTCAAGCATCGCAAGGCTGACCTT
AAACTTGCTATGTAGCCAGGGAATGACTCTGACTTTGGACACPCCTTCCTTCACCTCTGAGTTTTGAGATCACACGTGTGTACCCTGTCCTGTT
TGCTTAGGCAGCTCTGCAGAAGCCCCCTACCCAAGGGAGCTGTGTCGCCAGGCATTGTACTCATTCTTGGTGGGAAATCGGGGATGTAAGAGAA
GATGAGAAGCCTCCAACCCCATTACTAGGCACTGAGGAAACTCTGGACCTTTTCTGGCTGGTCTTCTTATCTGTGCCCCTTCCCTCCAAGGTGA
GAGACCGACATGAATCTCAGTGTCCCAACCCCCACTGCTCTTCTTCCATCTCGGCTCGG1GTGCTTTGTGAGCACAAGGCCCGATGTGCCCATT
CATCCCGACCCAAGGGGCATTTGCTATGCTACACAGAGCATAACAGTTGAGCATTCAAACACAGAAGGGCTCAAGAGGATGGCCAGGTCTCTTG
CCACTGTTTGCTGAGCCACAGGAAGGGACACATAGTGGTTAATTCTGGACATCTTACTCCACAGAGGTGTCCCCAGTCTTGTGGGACATGCCAA
TAAGTGGGAGACAAGGAGGACAAACAAGCCCTCAGGTAGCCCAGATATCCTGGGACCGCCTTTTTACAGGTGGTGGTGGTGAGACCCTGGTGAA
GATTCAGATACCCAGAGACTAAGTGCCTGGGAGTGGGAGCTAATTTCAGGAGGCCAGAAACACAGGCATCAGTGGCAGAGCATAGTTGGAGCTC
AAGGTGGGCACATGTTGATGCAGGGGGCAGCCTGGAGCTTTCTTAACTTAAGAGACTCCAATGCCCCCAATAAAACAGCCAAGTTGACAGGTAT
AGAGCAACCCTGTCAGCCTCTCCCCATCTGAGCCCCTGAGTCTAATTCTTAGTTTTTCTTTTCACTGAGGAGGGAACCAGAGGACTCACACCAC
AAAGAACCCGAGGGAAAGCCCTCTGCCCCAGTCAGCCAGATGAGGCTGGTAGGCAGCATGGGCAAATGGGAAGGTTGAGGTACAAGCTGAAGGT
ACTCACAGTTTCCTGCATGGGGTGACTGACGGGTTTCTCGAGGGCGGCCATGTTGTTGGGCATGTCCGGTGGGTGCGAGAGCTGGGGGGGACCG
TGCATGAAGTCGTTCATGGATGTCCCTCCGGGATTCCCATGGGGGAAGTCAAAGTTGCCATGACCCTGGTAGTTGTTTCCTGGGATGGGAATAA
GATGGGGATGGGAAGAGGTCAACCAGTGGGAAGGTGGCCACCCTCAGCTGATAGTATAAATGCCCAAAGGCTTTCCGTCCCTTGGGACAGGCAA
GGCTCCTGAAGCATTTGCTTTGGGCTACAGGATGGGGCAGTCATGCCCTTATCTCATGGACTGGGGCACTCCAGGTGTGTGCTAAAGTCAAGTC
CCCAACCCCACTCCAGATGTGTGTGTGCTCTACTGTTGCAGAAGGGCAGAAAGGGGTCTAGCCACCAGGCTCTAGATGATAACCCAGCCTCTCC
ATCTCACACCTGATGGTGATGATGAGACAGGGAGACTCAGAGTGCTGGGATGCCGGGGCCCTTGGCAAAGGCTGTGGGATTCCAGCCAGTAAAC
TAATGACTCTGGTCCACTAATCAGTCACCTGGGCACCTTGGAGACCTAAGGCCATGGATCTGAGTGACAAGGCCACCACCCTCAGATAGAAGGT
ACACGTGCATTTTCTAGGAACAGAGTGGAAAGCAGCAGGGATGATGCCTGAAGTCTGGGATAGCGGAGAGTGAGACCAAGGGATAACAGACTGG
GTTAGCCAAGGCTGGGCCGCCAGTGCCTAGGTGTTCTCTGTTAGAGGGTGGGAGAGCCTAAAGGGTGGAACCGATAATCTGGCAGAGCAGGCTG
TGCAACCTCTGAGCTTCCATCATCATTCTCCCAACTCCAGTGACCGCAGGGATGTTTTTGGGGGTGTGTGTTCACTCTTTCTCTTTTATTTTTA
AGATTGACTTTTTCTGTGTATGAGGGCAATCCGTTGTCCACGTGTATATATGTGGTTCCACGTGTGTGCCPGCTTCAGGGGGAGGTTAGAAGAG
AGCACTGGATCACCTTGAACTGGAGCTATGGTTGGTTGTGAGTCATAATGGAGGTTCTGGGAATTGAACCTACGCCCTTCTGCAAGAGCAACAA
GTGCTCTTAATGAGCGAACCATTTCTCCTGCC1CACTCAGCCTTTTGTTTCCTCATCTGTACCCGTTATCATCAGCACAGAGATCCCAAGTCCC
CGATGGACCCGGGATTGTACCCTTCTTCCTCCCTCCCTCCTTCCCTGCCCAGCATCACGTTACACACCTTGGCTGCTGTAGTCGTTGGAGCTGG
TGCCCCCAGGAGGAGGTGGGAGGGGGTAGGGAGACGGGCCAGGGCCCAGAGCGGCGATCATCTCCATGACATTGGGCATGATCATCTGGCTGGG
GCTCATGGTTTTGAACCGCTTTGAGGGGATGCCATCGGGGTCATCCTTGATGTGGAGGTCTGACTTGATGGGTACTGGCCGCCAGCTGCACGTG
GGGTCAATGGTGACCTCTTCAAACTCGGAGCTGATGGGGGAGAGCGAGGGGGAGGAGCTGAGGTTTAGAATGTCTAGCCAATGTCTTCACTCCT
CCCCTGTGCCAGAACTGTGCATGGGACCATCTTGTTTGATGAGGTGAGAATATTGAGGCTTAGAAAGACAAGTGGTTGAGCCTGGGGCTGCTGG
CCCCAAGACACCTTCCCCGCCACCCTAGAGGAGTTCCAGCACGGCTGCGTTCTGTCTCATCTAAGACATATTGGCCCCCAAGTCCGGGTATTCC
CAAGGTCAAGCTCCAGATGCGTGGCTGCCCAGCAAAGTACTCAGGACTGTCTATGTCTCCCTCCAGCCACGCTGCACCTCCCTAGTCGAGGCCA
CACTTACTGTTGGATGGCGTTCAGGATCCCCCACATGTACTGATCCACTTCCAGACCCTCGAGCAGAGCAGTTTTACTGGAAGAGAAGAACAGA
GGCTTGGTCTTTGCGCCTGGAGGCAGGACACAGCCCGCCCACCTCCACTGTCCTGCCACTGTGGATACAATGGAGGCACAGAGTGCAATATAGC
AGTGAAATCCAAGGCCCATGCCTCTGTGGGGCTGGTTTTGCCTGAAGGGTAGGTAAAGCAGCTAAGAGGCCCAAGAAGAGGCCAGAGGGCACCT
CTGCTCTGCAAGGTGGGTATTGCTTACTTGCACACGGGACACCTCCAGGTCCCTCGCTCACAATTCAGTTGCAGGTATGACTCCAGGTCAAAGC
ACTGCAGAAGTGAGAGACAGAGAGATAGGTAAGATCAGGACTCTCAGAAAATGCCTTGCTGTGGCTCTGGACTCTTGGGAAGACTTCAGTGCTG
GGGAAGAACTCTTCTCTTACTCCATAGAACCACAGAACAGCCATGCTGTGTCTGCCACGGCCCTCACAGATCTCTTCCCTGTCCTCGAGTAAGC
CCTTCTGGACCCAACACCCCAGCCAAGTCTCACTGCTTTTGCCTCTTACTGAGGCCGGGACCAGTGTGGACTCTGGCAGTCCCCTTAATCCATC
CCAGGGACCCAGGGTAACTCTGTTGATCAGCCTGTATCAGACAAAGCAATCACATCATGTCCGTACCTACAAACTCAGAGGGAGGCAGTCCTGT
CTGCCACTTAGCTTTCAGGATGAACTAACTGTGCTTCACAGTGTGGCGATGATGGTGTAATTGGCTCTGCCATCCTTGCACCCCTGTCGTGGGA
CCCTGTCTTGGCCAACCCCTACACAACCGGTCTTTGCTAGTCTAGGTTGCCTCTTTCTTGAACCTGTGGTGTCTAGCCAGGACTTTCTGCTGGC
CTACGATACCTGATTGTCTATTTCTGCATTACTACCCCTCCCCAGGGCCTCAGAGATGGACCTGAAGGAGAAAACAAGACTGAGAGAGCACGAG
ATACTGGCTGCCGATGGCCGCAATGTCTCTCAGGCAGCAACAGGCTCTGTTGGCCCTTGGCCTGCCCCTTGTCACCCTGCTAACTCTGTACAGC
CTGGGCCTGCTCACCTGCACATGCTTGCAATCGTGGCCTCGAGCAGGCAGCTGTATGCGCCGGAATGTGATGGGGCACTTCAGAGACACCTTGA
TGGCGGTCTGCTCCACGCCATCCTCCCCATTGAGAGTTGTGTTGCCCGAGGAGGCAGCCACACTGCTGAAATTCCGCTTGACTGTGAGTGAGGA
GGCAGAGGGTGAGGGCTGGGTGCTCCACTGAGGAAATTGGATCTCAGCAGCTCCTAAACACGGCTTGGATTCCCCACCCCTGCATCCCACATGC
CACACATGCCATGTGCAGGTGAGAGAGAAAGGACTAAGGAGGATCCCTTTTCTGAAGAACAAACCCTACCCCTGGGAGCAAGAACTTTCTGCCA
AGCTTGCAGAAACTTAGTCTGGTGCTGCTCCTGATTTAACTGGGTCAGCAAAAAGCCCCCTTAGGCGCTTTTGCACCTAACTGCAAGAGTAGGA
ATAGAAACCAGACTGTGTGGAGGTGTTCAGGGGCTGGGGGGGGGGGGGGGGAGGGGAAATGTCCTAACCCCAGCAAAGATTCAAAAAGGAGTGG
CCTTGTCATCACTAGTAGCAAGAATAGCCACCAATCTCAGAAGTCCCAGGTCCGCTGCTGGGCCTCACACCCACAAGCTAGGTGTCTGGAATTC
ATGCGTATAATCCTGTAGCCTTCCTCCTGGTACTGGGTACATCCCAGAGTCAGCTAATGAGTTCTCTCTGACTGCTCCCTGCTTCTCCTCCACA
GTTACCCTGGGGGTCCTAGTGCCCCAGCAGGCATGGGCATCCCTCCACATACTCGGCCTCCTGCTGACTTCACCCATCCAGCGGCCGCTGCTGC
AGCAGCTGCAGAGCAGCTGGCCTCCCTGAATTGTGATTCTAGTCCATATTCCTTGACCCTGTTTGGGGCCCCTTTTCTCCTCACTGTCTTTGGT
TTTCTGTAGTGCCCATGCTTGTCCTTGGTCACACATTGAAGCAGTGGACTCTAGGTCCCGAGCGTATCTACGCCTCAGCAGATGGCCACTCCCC
CACCCGTGCACCCCTAAAACCTTGCAGAGCTGCTGGQGTGGGAAGGTAAGCGATGTGTAAGCCTGCTGATGCCTGCTGACGGTCAGCTGGACTT
TCTAAGCCCTCACCCTGTTGACGCTCCCTTGGCTGTAGCTGCTGTAGCTGCTTCCGGAGGAGAAGGTGTTGTTCTGTCCATTAAACTGTTCTGG
CTGTTGAAATGAAATGAGACAGAGGAGTAAGCAGAGCAGCCCCGCGGCCTGACTCCGCTGGAGACACGAGGGTTCTGGGGAAGAAGGCACCAAG
ACGGGGGTCTCTAGGACTAGTGTGGAAAGTGGAGGGGCCCGAGCTTTGCTCCTGTTAACAATTTAGTAGACATTTTCCACAGCAGCCATGGGGA
TTGCTGAAATGAACACCCTTGAGCAAAGATGGCAAACACGCTGAAGGGTAAACTCAAGACGGGTTTTCCCTTCTGAGAATGCATTTTGCAGCAA
TGGAAAAGGTTAAAGGCTCAGAGATCTTCATCAAAGCACCATTGTAAAATCGAAAAATGAGGAGTGAATATGGGAAGGATGAGCTAGGGTGAGA
TCATCACCTCCATGGGCTGCTGGCCAGCCATTGAGACAGGGACAGAGACATTCTATAAGCAACAAGCATACAACATGTGCAGGGGGAACAGGCT
CAAGATTACAGTGGTGGTCAGCTTTGTCTTTCAGAATTGTGCCTCCTCTGCCATCATTGTCTCATGTACTGTGAATGAAGGAGAGGAAGGCTAT
TCTGAGCCCAGACACAGTTGAGCACTGACAGAAAGTCAGGACTGCACCCCCCACTCCTAAAGCTGTTACAGGTGTGTTAGTTATGTGGGTACCC
TGAAGATGGAACTCACCTTGTAATACTGCCCCATGTTGACTGTGGGGGGTGGGTACTGTCCGGTGCTGGCTTGGCTCGGCATCCTTTGTCCAGG
GTAGTTGGGAGATGTGAGTGGCCTTGGGGGGTTAGGGGTAGGGTACTGGCCTGCCTGGGTGGGGAACTGGCTGTTTGGTCCATATTGCTGGTTT
CCATAGTTGGGCTGTAGGAATGGAGAAAGGGCACGGGTTGTCATGGTGATGTGATGTACTCAGATTCCAGGTCTGGGCCCCAGCTTTTTTTTTT
TTTTTTTTTAAAGACTGATTTTATCGCCCATATATATTATCTGTATGTGCACTGTGTGCATGCCTAGTGCCTGTGAAGGTCAGAGGAATGTACC
AGAATCCTGGAATCAAGAGTTATGGATGATTATGAGCCATCATATGGATCCTGGGAACTGATCCTGGCTCCTCTGCAAGAGCAATAACCACCGA
GCCACCTCTCCAGCTCCCCAACTTCTCTTCTTAGCTCTATGGTCTCATGCACACAAGACGCCCTCTCTACCTGTCATAGGGAGAGAATGAGCCA
GAGAATATTAAAAGCCTCCACTTGCTTTGACATCCAGGGACACTACATGCTCCAGGTCCACGCCATATGCCATCACTCCATAAAACTGTTTTTT
TGGAACCCCACGGATGCTCAGAGCTTCTCAGCTGTGTCTTTGAGGGGTTCAATCTTAAAGTACTCCTTTGTTTCTCCCACTCTTTGCGTGTGCA
CCCCGATATGTCAGGGAGATGCAGGTCACAGAATTTGTTACCAAGAGCTGACAAGCCATGCAGTGAGGCTGATCTGGATAGCACTGTCCCTACA
TCCTATACAGAGCAAACATCTATCTAGTGCTCCATTCTTAGCAGCGTCAACTATACAGCTGTGGGAGAACTAGCCAATGAGGAGTGGGGAGCAC
GAGAGCCCCTCCTAGAGAAGTCACTGAGGAAGGAGACAGTGCCACCTCCTTCCATCTTCCCTAGCTGGAACAGTGGATGATCTAGGGCTTCCTG
TCACCTAGGTACCCAGAACATGGTGTCCCAGAGTCAATGGTTCTTTGATGTCTATTCAAGAATATTCCCTGTCAGAGAGTCTCATCCTCTAGGC
TTAAATCTAGTTGGTGAGAGTGCGCCAACTGCCTGAGAGTGCTCATCAGAGTGGACTGTGCTCAGGAACTTTCAATGACTGTACGGTGGGGGGA
CATCTGCTACTCTGGGCCACAGCGAGTTCTCACGGTGGTGTGAAGTGGCTGGTACCTGCATGTTCACCAGGCTACAGGAGTGTGGTAGGGGACC
AGACAACATGATTGAAGCCTGAATTAAGGATGTACCTTTTTTGTTGTTTTGTTTTGTTGACACAAGATTTCTCTGTGTAGCTCTGGTTATCTCA
GAACTTGCTCGGTAGACCAGGCTAGCCTAGAACTCATAGATCTGTTTGCCTCTGCCTCCCAAGTACTGAGATTAAAGGCATATACCACCAATGC
CTGGCTACGTTGTACCACTTCTAACATAGGATGAACATACTGCCATGAGGTAGAAGCCAGAGCTCCCAGCATCTTATAACCTGGACCAGCTGGG
AAGCAGCAATGCTCTGGGGAGAACTCCTTTAGTAAAAGCAGAGGGCTTGGCTTCAATGTAGGGCTTTCAATATGCAATTCTTCTGCGGCCTCAT
CTCAGGTGAGCAGCCTCAAGCTCAGACATGCATGCCCAGTGCAGCCAACTATCTCTCAGGAGCTTGAGGGTAGAGGGTGTTTAGTGACTGTGTT
CTGGGAGTGAAGCCATCAGCTTAAGGCCCCAAACCAAGGACAGGCAGGGCTGGGAGGAGCCTAAAGGCCTTTGAGTTTCTCTCCCTAGTGCCCA
CTAGGTCGAGGCTCAGGTCTTGAGTTGGCCTCCTGGGTGTACAGGAAGCCTAATCACTATAGCCTATTCCTGAGTCTCAGGCTTAGGGCACCTA
TACCAATTATGACATCATCCAACCTCTGTGAGAGTGGCGGCTGAAGGGACCTGTGGGGACAGAGAGCTTATGCTTTCGTGGCATAGAAATCTGC
TTCAGCTCAAGTTGAAGGCCACAGTCTTGACCAGCCAGAAGGACATGATCTGAGAAGATGAAGAGCTGAGGTGCCATGCCCAGGAAGGGGCATC
ATGGTGTGCCAAAGACAAACTTCACTGGAGCGAGGGGGGCGCATCAGCTGGGCACGGATCTTGGTCTCAGTTGTATTCTGAGTGTCACTGAGAC
CTGGTGACTTCCCTPTTCCCCTGACCTTCCACGGTGCTTGGGACCGTCTGCTGGGATGGTTTCCTCTGGCACCTCTGCTAGGAGACCCCAGAGA
AGGCACCATCCCCAGGAGAGCAATATGATGGAAGTAAGCCCTAGGGATACTGTGTGCCATCCGATGGCCTCCCTGACACACACAGCCTGTACTA
GTGTACTAGTGGCAGCCCTCACTCTCTAGGTGGCTCCCTGCACTGAGGTCCTCATCCAGGGTCATCCATTAGTATCTGAGTATGGACAGGGTTA
AGTGGTCTCCAPGGTTTCTGCTTCTACACTTGCCTGCTCTAGGCAGGGTATTTTACAATGTCACGGCCCCCTGTCCTCTGGGCATTCTAAGGGC
CAGTGTGGATTGTGAATACCATTGATTTAAAGTCATACACACTCAGGTGTAGTGACTTGTTTGGTGTTTTCCACAACAGAGATGATATTAGTCA
GGGGCTAAGCCTCCAAAGCCCTTTAGTTCAAGTTCTCTAAGTGGCCTAACTGTGCCTTTTCCAAGGTCCCTGGGTCCATGGTCCCTCTGTCAAT
GTTCCTTCTCCTCCCATCCCCCATCATAGTCACACTTACCTCTCCCGGGTATGGCCTCTTTATGCTCTGAATAGGAAGGGACTGGGGCCGGGGG
CCTGGGTAGGTCTGCTGCGGCATCCTTTGCCCGTGTGTGCCAAAGGGGCTGAPGCCGGGTGGTCGGGCCTGGTTCATGCCCATGCGAGGGCCGC
TCATGCCCCAGGGTGTCATGCCAGCTGCCATGCCGGCAGGGTTCAGGCTGCCCCCCATGGAGGCAGGCCCCCGAGGCCCGGGTTGGTTCATGAA
TTGGCTGTTATACGCCTGGGTGGGACCCATCTGGAAAGAGGAACACACCTTCAACGGGAGAAGAAGAAAAAAAAAAGAATAAAATGCCACATGC
ATTGAAAGTGCAGGGAAGGAAGGTAGGGGGCTAAAGGGAGAGCAGCACTTTCCCACAGGGCTGCAGTTAAGAGCCAGGAGCTCTTAAAGGACCC
AGTCCTCCAGGGCTCACTACTGCCCTCTCCAGGTCTCATGGGAAAATGCAGCTGGTGGGTTTGGGTAGGGCAGGGAGCAGTCACTCCAAGGCAC
AGGCAGCATTCCCAGGGAGGCAGGCAGGGGACATGGAAGTGGGTAGGGATGGGAGGGACACAGGGAGGACACAGGAAGAGGAGGCGGATGAGGA
TGGAGGCTGAAGCCCTTTCCTGAAAGCCCTGGCCTTGCCCTTGGTCAGTAGCCAATGGAGACACACCCTCCATACACTAAGTGTGATTTACCCA
GCCCACTTCCTCCTGCTCACCCTGCACCCTTCCACCCACACACAACACTCTTTTAAGGGGCTTCCAAGGATACCCGTGGCATTAACGCTCAAGA
TTTCCCCAGCCTGGGTGAGGCCAGGCCAGCGGGCAGGAACCCTTACCGGTCCATACTGGTTTATATCCTTGTTCTGTGTCTCTTGCAAGGCTGC
CACTGTAGCTGTGGCTGTGGCTGTGGCTGTGGCAGCGGCGGCTGCTACTGCAGCTGCTGCAGCAGCGGCCGCTGGCTGGGTGAAGTCAGCAGGA
GGCCGAGTATGTGGAGGGATGCCCATGCCTGCTGGGGCACTAGGACCCCCAGGGTAACTGTGGAGGAGAAGCAGGGAGCAGTCAGAGAGAACTC
ATTAGCTGACTCTGGGATGTACCCAGTACCAGGAGGAAGGCTACAGGATTATACGCATGAATTCCAGACACCTAGCTTGTGGGTGTGAGGCCCA
GTAGCGGACCTGGGACTTCTGAGGTTGGTGGCTATTCTTGCTACTAGTGATGACAAGGCCACTCCTTTTTGAATCTTTGCTGGGGCTAGGACAT
TTCCATAGCTTCTCTGCTTTTCCTTGGTCACCACAGACTACCTTCTAAGCAAACATTGACCTGAGAATTGTATGGTGCTGTGTGCTGTAGATGT
GGATTGAGGTGTGTGACACTCCTGTGTTGAACTGCGAGGCTAGAAACCCAGCAACTTTGCCCCGAGAGCTAGGTGGAGTGTGCTCAAGGTGGCT
TGGGCTGCTTCTGTCCCTTCTTCACTGTGATCTGAGGTTGGGGACTCTAGCCTCCATTTCTTCTTCATTCAGGTCAGTGAGCTTTCTAAGGAGT
TTTGGACAGATGGAGCTCACTcCCACTGCTCTCCAATCCTGCCTGCTCACCCCACTGTCTTAGCATAAAGGTACACGCCTGAGAAGGAACTGAG
GAAGTATCCCCTCTACCCCTGGGAGGTTGATGCCAATCTGCTAAGACACATGGCAGAACTGTCCTCATATCCTAGACCAGGCCATGTGGAACTG
GCATTCAAGTAAGAGTCCTTCCTGGATCTAGGTAGGAAGGCAGGGCTAACACAATGCTGTACAAGGGCACCATCTAGGTTGGTCACTCACCTGG
CCCCAAAGCCCCCGCTACCAGGGTAGCCAGGTCGGCCGTACATGTTGGGCTGGATATAGGGCTGTGCAGGGCCAGCTTTGGTGGAGAACTGTTG
CTGTTGCCCTGCGAACTGTGGGGAGTTGATGCCGGGCTTGCTGGTCCTCATGCCTGATGCCATGGGGTTGCCACCTGGATTCATAGGGTTGTTG
GCATTGGCCATAGGGTTCCCGAGGACCTGCAAACAGAGAGAAAGAGAGAGAGAGAGAGGGGGAGAGAGAGAGAGAGAGAGAGAGACTGTCATTA
AGGTCGGTACCTGAGGCAGCGTGGAGAAAGAGAAGACAAAGACTGAAACATGGCAGGGAAGACACAGCAGAGGAGCATGGGAGTGGCTTGAAGA
GTCCCCAGAACACGTAAGGGATCAAAGGGAGTGGGCTGAGAATGTCAGCCAGGAAGGAGTGAGGCCAAGGCTCACAAAGCCAGCCCAAGTGGTC
CCCATGGCTGCAGAGATAGATCTGAGGCAAGCGAAGTCTTTGGCAAACATTAGCCTACTCGGTCCCAACTCTACAATAGGGCTTGGTGGACGCC
CTGGGCTGCTGCTCTAAAAAAGACACCTCTGTGTCTTTGGCCCAGATGTTGTCTGTGGATGAAGACCCAGGGAAGCATGACAGCTCCCCCCCAG
GATGGCCTGGCCTAGGTCCGGCAGAGGGGGGCCCTCTGTTAGGAAGGAGGTGAGGAGAGAGAAAAGAAGAGGAAACAAGAATAGCATGTCTGGG
AAGACAGCTGGGCTTGTCAGGCTAAGGCACCCTTTACTGAGAGGTGACATCTTAGGAAAACCTAATCCAGACCATTCAGGGCCAAAGGTTTCCA
GCTTAGAACCCTGAGGTCAGTAACGCCCAGTAACGCTCTTGTTTGCACAACTGGAGGGAACAAGGTCTCAGAACAGTACTCATGGCCTTGTCAC
ATAAACACCCTTCCTCTTCTATCCCAGAAGGCCCTACAATGGGGCATGATCCTCAGGGACCCAGCTGTGTCCCTACTGAGACAGCTGTGTACCC
TTTCCGGAGTGATGCTGTCTGCAGCTGGATATGTCCTGAGACTGTCTTCAGTGAGGAGAGTGTCACGGCACGGTTCTCTGGAGAACCCCCCTCC
CCACTCTTGGGAGGAATAAGAGACACTTGTGCCTAGGAGCGACCATGGTAGAGGATGGGACAAGTTCTTACCTGACTCTGGGATGTGTTGGTCA
CTCCCCACACCGTGGTGACCACGGAGAGGGAGCCGGGAGGCTGATTGGTGTTCTGCTGCCAAGGGACAGAGTCATAAGGAAATGACCCATCACT
GCAAAGAGAGAAGAGGTCCTTAGAGCTGGGGCAACGCCTAGGTCGCAGTGTCTTCACTTCACTATCCCAGCCGACCTTTGCCCCAGTGAGCTCT
GGGGCTCCAGGCTCATGACAGCACTTGTATAATGGGAATGGGCCAGCCAGATGTGACCCTCTCCCAACAATGCACGGGCTACCCCTGTGTTAGC
TGTGTAAGCTGGGTGGGTCTACAGTCCAGCAGCTATGCAGTGAAGAAGCCAGCTAGTGCGATCGAAAGCTCACTCTAAGTCCCTTCTTCAAGTA
TACCGATTTCCCATGACCCCCTATAGAAACAGTCAGGACCTCGGCATTGTCTTCACTGGGGACAGCCTGGTTCCAACTTTAATGAGGCTGGAAG
GGCCTTGGTGGAATCTTGCTTCTGTCCTTGTTCTCAGCACCTCATGACAAACTAGCTGCTGAACTGAGTCCCACTGACCTGGACTTCGGAATTC
AGGCTTGGGTCACCCTTCCTCTGGGCCCACGACCAGGCCTGCCCAGGTGCCCCGCCCCACCTCCCAGTCCTGTGCACATCCCTGCCTGGCTGAA
ACCTTCCCTTCCCTACAAGTCAGCATCATCAGGCTCCAGGGTTTACTGGGTAAAGTCCAGATGCACTCCAGACAGAACGATCTATCCCACTGGC
TCGTCCCTGAGCCTTCACCAAGCCTCCTCACTCCAGCCCTGCAGTGGCCTGCTGATACAGGGAGCTCTTCCCAAGGAGCCCTCAGGGATCTATC
TACCTCCTCTACTCAGTAAGTCTCAGGCTTACAGAGAACACTCAGAAAGGCCTTCTAGCCAGCCAGGGTGATGTGGGAGGAGCACAAGGGGTCC
AGAGTGATGGACAGCATTGAGACTTGCCAGGGTCCCTAGGCCCACWCTAGATCACCCTTACTTCCTCAGTGAGAGCCGTAAAACACTGTACCTG
TGCCCCAGAGCAGCAGAAAATCTCGTGTGGCACCCTCTTCCCTGGGTCTGCCTCCACTGGTCTGTGCTGACTTGCTTCCTGTTTCTCTGGAAAG
TCGCCTCTTTGGAAAGCCTTCCCACCTCTCGGAATATAAAGGAGCAACCCACCCTGTTCTTTACGTATTTTTCATGACACTTATCACACATTTG
ATTATTATCTTCCTAGCCTCTACTCCAGAACCTGGAGTGAGAGGAATCCAGCAGTTTTATGGGACAGAGTGGATTAAAGGCCAGGGGACAAACA
AGCTAGCCAGGAGCCAGGCCCTGAACTGGCCCCGAGAGTGGAGAAGCACTCCTGAACAACCCCTGTATTTTCTAAGCTGGGGTGACTGAACACT
GTGGCCTGCGCTCTCATTGTTCATGGTGGTCACAGAGTAACCTACTATCTTAAGCTGTTCTGGCCCCTGCTGCCTGGTCACGTATCCGCCGACC
TGGAAATTAGTCCTGAACCCATCCCTCACTAGCTGACCCTGCAAGTCTTCCTTGGGGCACTCTCATTTTTGAGCACATGCCCTCATGCCTTGTC
CAAATACAGGAGTCGGTCCTGGTGCTCAGTAGCATGAGGGAGCAGAACACAGTGACAAACGGGGTCGTAGCTTGGTTGGGAGAGTGCCTGCCTA
GCATGCACGAAGCCTATGCTTWGCTCTCCATCACTGCACAGACACGCCCGCAGAGAAATATTTACACTTACCCTATTATCTCAGAGGAGAAGAC
GGGAGAATCGGAGGTTCAAGGTTATCCTTGGCTACATGGAAAGTTTGGGTCTAGCCTGGGCTGTATGAAGCCCCGTCTCAAAAACAAAACAAAA
CAATAACCCTAAACTCTAAACCCAAACTAAACAACACAAAACACAGGCGGGAGATGGCTCAGTAGTTAAGAGCACTGACTGCTCTTCCAAAGGT
CTTGAGTTCAAATCCCAGTAACCACATCGTGGCTCACAACCATCTCTAATGAGATCTGATGCCCTCTTCTCGGGAGTCTGAAGACAGCTACAGT
GTATTTACATATAATAAATAAATCTTAAAAAAAAAAACAACCACAGTGCAGATGAATAAATACTGTTCTCTCAACACACCGGCCACTGTGGACT
AAGAATCAGTACCAAGAGGCTCAGTCAAGAACACAATGTGAGCACAGGCCTTGGGGTCTGAGCAGCACATTCTCCAAGAGGATAAGAGTGAAGC
AGGCTCAGTATTCTTGGTGTCCAAGGAGGGGGATATCAGGGCTAGACTGGAGAGAGAAACGGCATGGCTCAGGAGCAGGGCCCAGGAGCACAGG
TGGCTGACTGAGGACTAAATGCAGCTTGTGAAAGTGAGGCCTGTGGCCAGGCAAGAAGGGAGGGGCTGAGCCACAGATGGCATAAAGTACCTGG
CTGGGACACCCTGCAGAGCGTGTTTTCACAGAAAAGGGAGACCTCTGGCCATCCATTATCCTTCTGGATTCCCAGCTCTGGTATGCTGGACAGA
GGTTCTCGAGTCACCGATCTCGAGTGACCGAATAAAGCAAAAAAGTCTGTCTTTCCCCCACCACACCCCACCCTTCAGAACTGCTTTACAAACT
CCCTACCACCTTCCAGGCTCGTGAAGATTTGATTCAGGTTTTAACAAGCAGCTGGACAGGTCCAGGGGGGAGGGGGGCCCCCTGTCTCTCATCA
TCTTGGCTCCCAGGCTACTGTATCTAAGGCCCCTACTACCCTCAGCACCATGGCTGGGCCTTGTGGGAGGAAACCACCTTTTCCTGTTCTGCCT
ACCACTGACAGAGTACAAAGCTAGCCAACATTTCCCAAGTGGGCTCTCCACTGCCTGGCCAGTAGGTTCCCTGTCTCAGAACAAAGGTGGCCAT
CTACTTAGTGTGCACAGGGCCCGGGGGCTGCTGGACGCATGCTGGAGAAGCAAGTGACAGGCACAAGGGGGGCGGACCCAGGCCTCACGACAGA
AGTGGCATCCACCAGCACACTGTGACTCATCGCCTTCTAAGGGCTCTGACTCGACAGGCAACCACAGCATCAGGCCTGTGATGTGTCACCTCTG
CCTGGACTCTCACCCCTGTCTCTGCAAACCAAGGACAAACCAAGCTCCTGTGCCAGCAGGGAAAAGGGACTGGAGTGGGAGGGGACTGAGTGGC
AAAAGGTGTCAGGGAGAAGGGCTCTGGGAGATGCATGCCAGGCCTTTGGGAGAGGCGTAAGCACTGGCCAGTCTCAGTGATCAGCACTGTTAGT
GACCTGAGGCTCCTCCTCTGAGCTTTTCTTATTAATTAATTAACTAATTAATTAAATGGTGTGATGGTGCTTAAGTTTAATCCCAGCACTCAGG
AGGCAGAGGCAGGTGAATTTCCAGAGGCCAGGTTGGTTTACAPAATGAGTTCCAGGTCAGCCAGAGCTACACAGTAAGACCCTGTCTTGGAAAA
GGGAATTGTGTGTGCGTGTGTGTAGAAACACTAGGCCACAGCACACATGCGTAGGTCAGAGGACAACCTGTGGGAGTTGGTTCTCTCCCTCCAT
GATGTTGGTTCCAGGAGATCAAACAAAGTAGTTGTCAGGTGCCTTTGTCCACTGGGCTACCTCAGTGGCCCTTTTTGCTCTCTTGTTAAAAATA
ATTTTCTGGGGTGGGGGCAAGAAGAGCCTACTACCAACAGAGCCTGGTACAGCTGTGCAAAAAATAACAACTCATACAACTTGTGTTTTTCCTT
TTGTTTTGAGACACAGCCTCACTATGTAGCCCAGGTTGGTCTTGGATTTTTGACCCTCCTGCACACACCTCTCCTGAGTGGGATACAGCCTGTA
TCTTTGCTACCCTTTGGTTTCGCTTTCTGTAGTGTTGGCAATCGAACCCAGGGCCTTGGGTTTGCAAAACAATACTCTACCACTGCCCCACCCC
CAGCCCCTGCTTGCTCTGAAGGGGCTGGGAAGGGACCAAGAGCCCGTCAAGAGGAAATCACGCTAGTGTTGGGACGCCATACAAGTCCCTGGGG
ATCCCCGTGCACCAGGCTTGGTTCTACWCTTCTTCCCACAGATGAGGAAACPGTGGCTCAGAAACTGGTGTCCAGTCTCACGGCCCCATGCCAT
GCCATGCCTCCACCTAGCATTCCGGGGCCTCGCTTTAGATTGTAGCAAATCCCTGGCCCTGGACGAGGCAGGACCTCACCCTTGATCTCAGAGG
CTCCAGCGGCTTTGATTGTAATCAAATTAATAGATAAACAAGATTTATTAGACTCTTAATTAGCTTTGCGACACCGATTTGCATAGAGACAATG
TGCTAATCACAAATGGCTTTTTAAACCACTCATTAAAAACCTGGGATGAGGAAACAGACGTTGTTGGGATAAAGTCCAGATCACCTCAGGGACA
CTGAAGGTGTCCTTCTGCCCAGGCAATGGCTAGATCCTCCAGGTAACACACAGCTTCTGTCCTGGAGTTACGGTGGGGGTGGGGGGATATAGGA
TGTTTCTGTGGCTTCTGCTAAGTGGAAGATGACCTTGGTATGGTCAACGGTGAAGCTAAGACAGAAGCTGAGGACTTTTACAAGCTACAGTTAG
CTCATACTCGTCACAGGGTCACACCTACTCTTATCAAGTGCTTTGGGAGCAGCCTGGATCTTCTCTGCATTTCTCTCATGGTCGTGGGCAGCAC
CATGTCTTTTTGGAACCTCAGGAGCTCAGAGAGGTCAAGGGCTCTGGTTAGAGGCACAGCACAGCCTAAGGTGAAAGAACTTGGGCCCCAACTT
GTGGCCTCACCCAACTGTGGCACCACCACATCTACATGGCTTGCAGAGGGTATGATAGTGCTCTGAACCACAGAAAGACCATGGTCCCCAGGGG
CCCTCAGACCTCTGGGTTCAGGTAGGGTTTCCAGCTCCAGTTGAGGACCCAGAGTCTCTAAATGCCTGGCTCCTCTAGGGACCAAACTCCAGAG
TTCCCAGTGTACAGAATACACCCCATCCTTAGTAAAAAGACAACAGCCCCTTTTGAAGATATCAGAGTTAGTTTCTCAGTTGCAAATACTTGCT
TTTTGCTTCCACTATCTATGTTTAGGAATTTACATTAGGGGTTTAACAGGTGCTGGGGACCTCCAGAGTTCCCCTGGAAATGAGAGATTTCTCC
CACAGGGACATGCACGAAGGTTACAAGGAGCCGCCAATCAAGTCAATTCCTGGTCACTGGTTACTGAGTATCCTCTCCACCATCCTGGGTTTGG
AGAAGGGTCAACAATTGCTCCATCATAATGTGGCAGGATTATGATAATTACCTAGGGTAAGCCATGGATCAGAGCAGGGCATAGACTGTTTCAG
AACCCTCACAATCAGCAAACAGCTATACCCTCACAACCCTTGCACACACTCTTCTGTGTCCTACTTCAACCCTATAAGCTGTGTGTTCTTGGAC
AACTTACTGTCCCACTCTGGGCCAGGTTAGTTTCCTCTTAAGTTCTGTGACCTCGCTATGCTCACAAGAGGGACCATTGCTCAATTCAACTGAT
CAGCCTCAACCCCAATCAATACCCAGCGAGTGCCCAGCTCCTCAGCCTGTCTCGGAAAGGCCATCAGAGACCACAGAAGACATGGTCCCTAGGT
CTGTAAGCTTGCTCAACACCTGCTCTGCATTGTCTGTTATCCTGCTGGATCTTCAAGGCCCTGAACCTGCCATGGTAGCAGAAGTAGCAGGACC
TTGGGCGGCTTTTACACACAGTATTTTGAAGCAAAACTAGATTGAGGTTATAGGATGCAGCTGGCTTCTCTTCAGCCTTGGATCCTCCATGGTC
AAGACACTTTCAGAGCCCTCCACAATCATCTCTGTCTGCTCCTAGACACACGACCCCCGCTTCCCTCTTTACCCTGCCTTTATCCACCAGCTCC
TCATTTTGCCCCAGGAGCCTTCCTCCTCTGTTGGCAGAGTCAGCTTCTAAGTAAAGAGACTGCTTTTCTTTGTTTAATGACAGATAATCCGTAT
CAGCTCAACTTGATGAAGACCTAGCAAAGGACTGGCTGTGGTCAAAACCTGCCAGCCTCCAGGGCTAGAATAGCCACAGCCGAGCAAGTCTGAG
ACAAGTCTCTAGTGGGAAGTCACACTATTCTGGTCTATCACCATTACCAACAGTGAAATGACTCAGGCAAGAAAGTTGGGAGGGGCAATGTTAA
TGGGGTGATGAGAGTCCGTCCTGATGGTTCTGAAGCTCTGAGGGGCTGGGCAGAATTAGCAAGACACAGCTGAAAATGATGGCAGGAATATGAT
GGCAGGCTCGCACGCACACACGCGCACACATGAGTACATGCTCTCACACATGTACACACATACTCACACACCTGTAATCCTAGCACTAGAAGGC
TGAGGCAGGAGGATCAGAAGTTGGAGATCAGCCTGTGCCACTGAGAGAGACTCGGTCTGCCTCCAAACACCAAGGAAATGGTCAACCACCATCA
GACCAATTGCCAAAGTCCTATTCACTGACAGCTTGGCCAGGGATAAGGAACAGAAGGCAGAAGGAAGCAGGTTTACCTCTCTAGAGCAATGAAT
GGTTTTCAAGTAAGGCGGCATCCCCACTTTCCCAAATCTGCCGGGTGTTCTTACTGCTGCAGTGCTTAGGGAGTGGGGAGGCAGTCTTCAGAAA
GGTAAGGTAGGCAGGGCCCATGATGCTCACTCCATTCAGCTACCCAGGTCAAGTGCCACCCCCTCAGGAAACGCTGGCACAGGAGATAGAGGCT
GGGTTAGCAGAGCCTAGTGAGGCAGTGTCAGCGTGCTGTATAAGGCCTTGCTATGCTCTGAAATGCTTTTGACCATGCCAGGTAAACGTAGTTG
GGCCACCCTGGCCAGGCAGAGCACTTGGCTTTCATGTGTGCTTGAGCTCCTCTCTGGTTTCGATTTAGAGGACAAGGTTGACACCAAGTTCTGG
CAGTGCCCGGCGGTCCAGAACTGACTACAGGATGTTCTGGGCTTCAGGTCTCTCGGCCAGGCAGAATAATGAGAGCTGAGCCGACTGCAGAGGC
CCCAGTCATTAGCGAGGACCCACAAGGGGAGTGCAGACAGCATGACGGGCCCTGTTGTTGCAGGGTCAGCCCACAGAATACTCAGAGCATCAGC
TCAGCAAGGCGCCTCCGAGATAATTAACAGGGAATTGCCAGTCTCACCACGTGCTTTTCTGAGGCTGGTGCAGGGGAGCCTGGCCAGTTCCTAG
GTGCTTGCCAGTCTTGCAAGCCTGTTCCTGCCTAGGATCTAGGGTTTTGGCAGACCCAGCTCAGTTTCACTTGTTCCAGTATAATGGGTGTGGC
CTGGTTCTGCTCTACAGTGAGGTGAGGCCATAGCCCTGGAGTGTCCTGAGACCCAACTGGCTTTCAGTTTCCCTGAGAGTGCTTAAATTCCTGC
TTTGGTTAGAGGGAAGGCTCCAAACTCATGATTCTTAATGTTCTATAGAGGCATCTGAAGGACAAGGTGGGGAAAAACGTCTTCTCATCCCTGA
CTGACGGGAAGACGCAAGCCATTTAGGAGGATTTAAAGGCCAACTGCTTCTCAGAGGCCCCGCCCTAGGTATTACACCACAGGAGGTCCCCACC
CCCAGACCCCAGGCCAGAAGGCACCTGGATGCTCACCTGTGCGACAGAGTGGGTTTCATGGAGCTCATGGAGCTGAGGGGGGGCTGCATGGGCA
GTTTCCCAGGGGGGTCGTTCTGGCGGCTCTTCTGATGTCGCAGCAGCAGCAGGCGGCCAAGTTCTTCACACCAGGAGGACAGCAGGGCTGCAAG
CAAAGACAGATGGTCAGTGGACACACTCGAGCCCCCTCTGCATCCTTCCCAGACGTGGCGTTTATGAGGCCGGTTTTGCTCCTGGGAAATTTCT
AAGGCTTCCTGGTAGGCCTCAGTGTTTTAAACTATAAAATGGACCAAAGGCAAAATCAGAATCATTTGTGACACGAGTCCACTACAAGGGCCAG
CTGGAATGGCCACCAGGTTGAGAGGCCCCAAAGAACAGTCCCGACAATGAAGAGAAGACGGAGCCAAGGGTGAGATGAGGGGAAAAGGCCAACA
GAGGCCTTGGATCAGGGTATGAGCAGGCACTTCTAGGGAACCCCATTACCACATACTCAGGACATTTAGTGCTCCCATGGGTGTGTTAGAAACT
GTAAAGTGTAGACATAACTTAGAGCTTTCTTGGAGTTGCTGAAGACCCAGAAGCATTCGCTAGACTCTGAAACCCCCCAGCCAGAACTCTCCTG
TTGAGTACACATCAACCTTAGACAGACCGACCGACAGACAGACAGACAGACATGCACGCACTCACTCTCATTGGACTCAAATGCTTGGAAGGAT
TGAAGGTTTCCATAGCAGAGGGTGGTAGTGTGTGAGTGTGAGGTGTGAGTGTGTGTGTGTTTGCACTAGAGACAGCCAAGGCAGCATGTGCAAG
AGTCTCTGCCACCAGCCCAAGACTACAGTGACCAATGCAGGGGGAAAGAATTGTCCTCTCTTCATCCCAGTGTCACCACTAGGGACTAAAAGTT
TCCTTAAGGTCCTTTCAGGATACAGCCAAGTTTAGGAAAGGGACAAGGTTAAGGCTGGGCTTGTCACCACATCCCCTGTAAGTCTCAGAGTACT
TGGAGATAAATCGGGGTGTGGCTCCAGCCGCAGATGATGGCCTTGTAGGACAATGAGAGGAGAGGCCCTAGGTCCTGTGAAGGCTCTATGCCCC
AGTGTAGGGGAATGCCAGGACAGGGAAGCAGGAGTGGGGGTGAGGGGTGGGATGGGGGGTAAAATGTCTATTAAAAAATATTTTTTTTCACCAA
AAAAAAAAAAAAGAAAGAAAGAGAGAAAAAAAAAGAGCGGTATGGATTTTTGTCACTCAGTGCTCAAAGCCAGAGTGACTTTCCAGAGGTCGAG
TCAGCGTCCCTGCTCTGGAAAATCTGCCTTCAGCAGATAAGCCGCAGTTGACCTTCCTCTCTTGACTCCTCCCACTTAAATAAGGAAGCTGAAG
TCAGCACTGCTAACACTGGGGGACATGCCCGATGCAATCTGACCAGGACTGCTGGGTGCTTCAAGCTGCCAGGCCCTGCACGGGTCCTCTCTCA
GCCCACAGCTCCGACGGCTATCACTGGGGCCGTGCCACCAGGTGCTTGCTCATCTTGCCAGTGTCCAAAAGCTCCTGACCAAGGATGGAAGTGG
GACTGTAGCCCTCAGGAGAGCACCCGTGTCTCCCCTAGAGGCTGGTGGATATGCTGATGCAGCTTGTCCTTCTCTGTGGGTGTTCTCAGTGTCA
CCAACACAGGGAGGACCCTGCCCCTGGCACGCATCTGCTGGGAAGCCGGTGAGGCTCCTAGTGGGTGCCCAGTGTTCAAGCGTGCCAGGAGGGA
AAGCCGCCTATCCAGAAAGGCTGGATCAGGAACAGGTATGTTTTCAGATTGCTCCATTCTTTCTCTGGGCCTCGGTTCGGCTTCTTGCACAGAG
GAGCAGGCACCTAGAAGCTCATTTGCACGCCAAGCACGAGGCCCACAGGGTTGCAGCTCTAGGAGGCTTGCTGCCAGCACAAAGGATGGATGCC
GACCGGCTGCTTGTGACACGCCTCACTATCTTAGCCCTGCCCTCTGGGTACCAGCCAGGCTGCTGTGGGATTTAGGGTTTTCCTTGGTTACCGG
AGGCTGCCCTACAGGTAAAGGGCAAGGTTGTTCACTATCCCAAGTGGACTGGTTTCTGGAAAGATAATGAAGGCATAGGACTGGCAATGGAAAT
GGGAAGGAGGTGTTTGTTAACTATGTCTTCTCATGCCTGGAAGGCCACAATCCAGTGACCAGGAGGGAAAGAGGGGTGACACAGAGAACCCTCC
TCCCCCTACCTTGGCTAAAAGCCTGGGGAGCACAGAGCTTGAGAGCAGCCCCCTCACAATGGGCAGGAGCAATGTGCATGCCTACGAAGGCTTG
AACGGACAGGGGCTATGTGGAGACAAACACCACATGGAAATTTGCAGCCAAAAAGGGAGTATGGGTCTGTTTACAGACAGGCCAGGCAGGAGCC
CATGGGGCTGCAGTGCTCCAGCCTGGTGGGACCAAGCACACTTTGATCTGGGAAGGCCTAGCTGCTTCCTGAAGGTCAGTTTTGCCTGCTTGTC
CTGTGTACCAGTGGCAGGTAAGGCTGCCCCAGGATGGGCAACAGAGACTGTTCTGGTCCTAATTTACACCCTTCTCTGTGGTAGATAGGTCTGA
AGTGCTCTATCAACTGTCTAGACAGCCAGCACTGTTGCCACTCGTCTTATCCTAAGAGTCTCACTCCCAAGTTGACTACAGCCAGGAGCAGTGT
CCACAGAAGCCCGTGATCTAAGACCTGGCACTCAGAGGCATGACCACAATGGCACCCACTGCTTCCTCTTCTCCCGATGGACCCATGGCAGGGT
TCGACTGTCCCGAGAGCAGAACACATCAAGCCACAAACTGTCTTTTGTCTACAG1GCCTGGAGAACAGTCCTTAGAGGCCCCTTCCTTACGCCC
ACCTGGAGCAGCAGGCCTTACACCACCCATGTCCAAGCACTGAAACGGGTAGCCCTGGGAGTCTGGCCTCTCAGCCTGGAGGGAGCCAGTTTCC
TCACTAGCTCATCAGAACATTCCACCAAGTGTCCCAGCTAATATCACCACGAAACAGTGGGACCCACATTGGAGCCCAACTGTCTACTCCAAGT
CTCAACCCAGATTTCAAACTGTTTCCAAACTTCTCAACATGGTATACAAAGATGCATAACCCAATACATCTAAAATGATCATCAAATGAGGGCT
GGCAAGATGGCTCAAGGGTTGACGGCTTGAGTTCCACCCTTGAGACCCACATGTTGGAAGGAGAACAGACTCCTTCGTATTGTCTATTGTCCTC
CTCATGTCACACACACACACACACACACACACACACACACATGGATATATATACAAATAGATGTTAATAATTTAAAAGGAGATCCATTGACCAT
CTGGGGACAGGTGTCACTGTGTAGCTTGGATGACCCAGCACTCACTATGTTATGTACATCATGCTGGCCTTGAACTTCAAGTTCTGCCTGCCTC
TATTTATTGTTGAGATTAAAGGCATATGCCATCAAGTGTGTGGCTCTCCACAAAAATGAACTAGCATTTTGTTACTAGGTTCCCTTCCTATTCA
TTCTCACCTTAGGTGCCAAGCGTCTGATCGCCCACCTATATTAATTTGACAAAGAGCTGCTGAACACTTACTGAGTGTAGAGCTTCCTGCACTA
GATAGGATGCCCTGTATAAGCCAGCAAGCAAGGAGGCAACTCTGTGCACTGTGCACACTCCTCTGGCAGTATTCAGGGGTCAGCTGTATTCAGG
GTAAGGGACAATCACAGATCCAAACAGTTCCACCTGACTTCCACGCAACTGTTAGCTAAGCACTCACGTGCTCTCTCTGAACCTTTATCTGTTG
ATGAGTATGACCCTGATGCCTGCTCTTCAGGATATAAGGTGCTTAAAACAGTAGCTTGTAGGTAGAGGGGTCTATGTAGCATTGTGGCCCCAGA
AACCTATCACTCACCATGTGAGCAGTGGGAGACAACACCCTGAGAAACTAGACCACCACTAGGTGACACCTAAACAAGGTCCCAGGGCAGCTGT
GAGGGTGGTCTCAGTCAGCACTCTGAAGTACAGGAAGACTGCGGACCCATGGTACGCTCATCAGATGATGAGCATCAGCCCCCAGAGTTGACAG
AGCTACAATAAGGAAGGCTCTCAGGCCTTGCTGTGTCTGTCTACTTCTGATCTCTGAGCAGAGTGCAGAGCCAATGTCCTGGGTGGGTATGGGC
TCCAGACACACATGGGAGACCTGTGGGTAGACGGAGGCCTCGGGGCAGATGATGACGCCACCTGCTCACTCAGTACAGGCTCTGCATTCCGCCC
TGTCCCTCCCTTCTTGCCCGAGCTGCCAAGGGGCAGAGGAAGGACAGCCAGTGTCCTACTGTGAGACAGGAAGAGAAGGGCCATTTCCTATCCA
GGTGAACCTGTGCCTCTTCAGCGGTTGCCCCAGAAAGACAGTTTGCTCTCCTGTGTCTCTGAGGACATAGCTAGGATCCTCTGTACCAGTGGTA
CTGGGGAGAGCAGTAAGCACGGCATGCCCATGACTACTGAAAACTAAAGCATACAGGAAAGACACACGGACTGGTGACTGACTGGGTCCCAGGA
AGTGGGTCTGAGGGGATATAGAAGAGCCAGGTTCCCCACATCTACCTCTTCAAGATCTTTCTAGCAACTACTCTCACAAACAGGTACTATCACA
ATGTCCTTTAGGGCAAGGAGGGGAACAACGGGAGAGGCCCCGAAGACTGGAATGCAGAGCCCTAGGCACCGAGCTCGTGACCGACCCTAGGTGT
GCCTCCCCTAGGGGCAGGGACAGGCCCTGAATCTGCTTCTTTTTCTGCACCTCCCAGAGGAGATCAGAGGAGGCAGAAGCAGCAAGGCTCTAAT
TTGGGGAACCACCTGGACACCTGGCGTTGTTCGGAACTTCAGAGATCAGCTTTTGGGGGCCAATGAGGATAGGATCCAGTTGGGGTACAGGGGA
ACAGAGGCTGGATCTCAGCATCTAAGACAGTGAGTACACAGGGTCATAGGAAAGGTTGAACCTCACTAGTCTGGGTTGGGCTTGAGCTTGAAAA
GGAGCAAATAAACCAGAGGGAGCCAGATCTCTGGGCAGCTTGGCAAGGTTTAGATACAGGCCCAGGCTGCTCAGTGCTTTAAGATCCAGGAACT
GGGAAGGACCAGATGCAGGGAGGAGCCTGAGGAGCAGTCTGGAGAGCAGGGAGGTAAGAGACAAACAGAGGCTTTGTCCACCCTGCTGCCAGCA
GGGAATGCACAGTGAGACAAGGAGACTCTGAGGAGCACAAAACATTTCCATGGCTAGAAGGCACCAGGGGAGAAGGTTGTGTGTGTGGAGGGGG
CAGTAGAGATAGGAGGGGTGGCTTCTGAGTCCCTCAGGCCACCAAGGATGGACTCTGAAGTAGCCAGGAAGAGACCGTCAAGAGACAAGGGATT
CCTCCTTCACACATCCTAGGAAGGAAAGAATGCAAGGAAGGGCCATTCCTCCAATAAATAGCTCAGTCACGCATTTCAGGTGCACAGGGGACGA
TGACACAGCAGCAGGATGAGTTAGGCACTTCCTGGTGAGTCAGAAAGCTACCAACATGGCCACTCTAGTCCCCCGAGCACACCTACCTATCTCA
AGAATATGTACCAACATGGTGTGTTGATCGTCTCAGCAGCCTGCTCTCCTTACACCTTCTCTACTACAAGCTCTGCAATGCATCAGAGCAGGCC
CACCCATCATGTGCCTGTCTCCCAACGTGTCTGTCACCAACCCACCCTGCTTTGGATAGACAGGATAAGCTGAATGGAAGCAAATGGGGCCAGG
AGACAGGCTACCCAGGGGTTAGACATGAGACTGGAGCGGCCCCTTGCATAGTCCTAGCACTTCGCATAGCATTTAGTAAGAACTACGCAGCCAA
CACATGACTAACAGGTTGCAGTGCGGCACAGACAGAAATAGCTCGGTACATGAGAGTATGGGAGACGGTCAGTCATCAAGCACTCCATCTGAAA
GCCAGGCTGTAGAGCTGGTGCAAGGCCCCTCCCGGACTGCAAGCCAGTCTCCATGGCACTGTCACCAGATAAAACCTATCACAGAGTCCACTGA
CACATTTCATGCCATGCATAGCCACGGACGGCTAATGAGACTTTATAAATATAAGCCTCTTACAGAAACAAGAGCGTAGGTGACTTAAGGACAG
CTGCCACCTTCATGTCATCTGTCCTGGCACCAGTGCTGATGGTGTCTGTACATCCACTATGTGGTACCCACAGAGGAAAGGCCACCTCTTATCC
CTCTAGGTGGGGCCAGATCACACTCTCACCTTCTCCTCTTTCATCTCCCAAGCACAAAGTCTTGGTCCCAAAACCAGGTGGCTGGGAGTCTCTA
GCCTGGGCTGGCCACTCTAGAACGTATCAGGGATTCACAGGCCTCGGCTCACCCCTTCATTCTGCGTGTGAAATGGTGAGACAGCAAAACTCCT
CTTGTTTACAGACAAGGATCTGAGGATTCTCGAGGCAATAGACCAGGCTTAACTGTGGAAGCTCACAGGAGAAGCATCAGGCTTCCTCCCTCCT
GAGGTCAACATTAAACAAATTCAGATTCTGGCAAGTGGAGTATCCTGCAGGATAAATTCACTGGTTTTCCCTGTTGCCTTCCTCTGAAGGCCTT
CCAAGGCTGAGACATCTGACAAACAGAAAACGAACCTGGTCCCTTGTGGGTCTGTCTTTCCCACACTCTAGGAGAATCACATAAACATACACAG
ATGCTCAAAGCTGTCCACTTGGCACCTTTATGTTTGATACTCCCTGGGGTGGGCAGTGCCATCTCTGGCACCCAACAGTGGCCACATCCAGTGC
TTCTCTACAAGGCTGACCTCCGACCCACGTTTCACCTTGATCTCTACCTACTCCCGCCTCATCTCTTGTGCCAGCCTATTGAGGCTCACCCTCT
GTAGGGGCACTGAGCACAGGGCCTGTCCACACAGACCATCTCTGAAAGGCTCTTGGCATTTACCCTATTGTGCCCTGAAGGGCGTGGGAGGGCA
CCAGGAGCCAGGCCTCCTGCTCTCATGGCATGGCTCCCTTGTAAGAGTCAGTAGGTTGGACTTGTGTCCTGAGGAACTGAAATGTCTGCAGAGC
TGTCTCTTGGCTTCCTTCCTGCTGGCCTCTGTAGGGGGGCCCCAGCAATGGAGGAATGGTCCCACTCTCTGCCTTGGGAGGAACCCACAAAGGA
ACCCCGTGACCATGAAGGAGATTCTAGTCTTGGGCTGGGGCAGGTAGCTCTGAGTCACTGATCCGGTAAAGCAGCCTTAGGCTCTGCAGGCACA
TTTGTCTACAGGAAGGGTGGCCATTCTGACCCTACTCACCCTGCTGCAGTGGATGGGGAAGATCTGCCAGCCACCACGCTTCCTTCCCCCACCC
CCTCCCCAGCTACTTCACACTCTCAGTTTGACTCCCATGCCTTGAGGTCTGCCCAAGAGCTATACTGTATTGGAGGAAGGGGGCAAGGGGTGTG
GGTGGATTCAGAAGATCAGGACAATGACCCTGCCACTGGTACCTAGGACTTCCCTTTTATGTCCCTCATCTGGGACATCAAACCCATCACTAGG
AGACACTGAAGTACCATCCTAGTTTCTCATCTGAGCTGAGCTCAATACCTGTGTGGCCAAGAGTGGCTTTTGTCCCTTTCTAAGTTCAGGAGGG
TGTGTGGGTAGGTGGCAAGAACTTGGCACCCTTAGCAACAGGGGGTCAGGACTTCTTGCTGGTCACAGGGAGCTGGACTTTGCTGGTGCTGACA
AAAGCCCCTGGTTTTCTGTGGAGGAAGTACTCCTGAGGAATTGCCACACTCTTGCCAGGCCTTTCTCAGTAAGGTGTTGGCCTAAGCTTCTGGT
TTTCCCAGATCACTCTCACCTTCTGTTCCCTGCTTTGCCAGCCCCGGCACTGTGTGGTGTGAGAGGCAAGATGGCAGAAGCTGCCTGCTGACAC
ACTGGCTGGGAACCCTTCCATTTATCCCTCATTCTTATAGAAGCTTAGGCCCCACAGGCCAGGACTGGTCAGGTCTGGTCCCCTAACTGCTCCT
CAGGCTGGGACAGTGTGTGCTTAAAGAATCCTATGGAGTGACCCAGCCTGGAGTAGCTTCAGGCTACTCCTCCCACCTCCACCCCTGCCTCTAG
AACACTCCCTCTTCCCAAGGGCTCTATGGGTGACAGAAGCTGGAGTCTCTAGGTGTGTGAACACACCCTGGGAACTGAACTCTGAGGAACCTTA
GAGCCTGCAAGGCTGATTCCCAAACCGGTTTGGCTGTTCCTTGCCTTGTGGAGGCTGTCCGGGGCTGAGCAAGAGGAGACTCAAGAAGATTCCT
GAAGAGTTGAGCCTCTGGGGGCCTCAGAAGAACCCAGTCTGAAAAGTTCCCTGCACATTTCCCATTACCATCAGGTCAGCCTATATAGTTCAGG
CTCAGATGCTAAGACTGCAGTGCCAAGTCAGGGGAGAATGTTCATCAGGAAGAGAATGTCCTACCCTTAGGATGGTTTCATCTCTACAAAGTGT
CCTCTCAAAGTACTGTGGACCCGAGGACGGGCTTGGGAAGTGAGCTGGCAATCTGGCTCAGTTCATCTGACAGAGAGCCACTTCTGGAGATGTC
CCTCTTGAAGTAGTGGCTCCCTGGTCCTCCCACAGATCCTCCTGCGGGTGGACAGCTACAGCTGCTATCCAGGAGAAGTCAGGAGCCTGACTCA
GGCCACTGCTCAGTGTCTGGCAGGTCGTGACCTTTGTCTTCTTAGGCTTTAGGGATGGCCATTTGCTGGCTAGGGATTGCAGACCATTACAGTA
GGAGACGAAAGAGCTTTATGAACAAAGCATGGGGGACCAGTGAGAAGGCTCAGTGGGTGATCGTGCTTGGTGCCAAGCTAAAGTCTACTCAACC
TTGAGTTCAAATCCCAGGACCCAAATGGTGGGAGGAGAGAAATGGTTCCCACAAGCTGTCCTTTGATCTCCACACACAAACATAGAAACGTAAA
AAATAAAACACAAATGACAACAGGCCTTTAGTCCCAACTCTCAGGAGGCAGTGGCAGAAGCAGGCCAGTTTCTCTTGAGTTTGAGGCCAGCTTG
GTCCACACAGACCTTATCTCAAAAACAAAACAATAACAGTACAATACCAATAATCCAAGTGCTGGGGATGGTAGCTCAGTGGCAGAACTTTTGC
CTAGCATGCATAGGGCTCTGGGTTTAATCCCCACTACTACAACGTACAGCCACAGAAACAAAGTATGCAGCACTGAGGGGAAGGGGAGGGGAGG
AGGGAGGAGGGAGGTAACTATAGTCTTCCATTTAGAGCCTTGGACCTACGCCCATAACAAACTTCTCTCCACAGCCTGGGAAGACATGGATTCC
ACCAGGGCAGAACTCCTTGGTGCTCAAGGACACACACTGGTGTTCCTAAGGGGCTATTTGAGGACTCCCCACCTGCCCCTGCAACTTCTTCTTG
CCCTAAGGAACACCTGAAAAGGCCAAGGATAGCCCAGAGGTGGCAACGGTAGTAAAAGGGATGTAGTTAGATGGCCTAGGAGACCAGAATGCTC
TCGCATTGGCCTGAGTCCCTAAGGTGAGAAGACACCCCCCTCACAGACACACACACACACACACACACACACACACACACTTTATTGATCGTGG
AGCTGCAGCAGGGACAGCCTCAAGATCCATTTTCCTTCCTGAACACGCTCTCTCCTACAGGCTAAAACACCACTACCCTCTGTTCAAACTTAGG
GAATTGGGGGGCCAAGGAAGTCCTATGGAAATCCTTTGTCCCTGGGGAACCTCTGAGACATGTCATCGAAACTCACGGTCTGATTCCTCCAGGT
AAGAGGCTGGAGTGAGCCCCACTTCTTCCTTAGATAGCACAGAATCCCCAATTCTCACCACATCGGCAGCCAGATGACAGCAGATCACACCTTT
ATGTGCTGAGCAACCCAGCTGTACACTGTGTCCAATCCTAGGGGAACCAGCAAGCAGCTGGTGACCACTCTCAGGCCCCTCAAATGGCAGAGCT
CATGACACCAACATAGAGGCTCTGTGGGGTGACTAAGTCTTGGTGAGATGATTGCCCCACAGCAAACTTGTATCAACAAAGGAACAATCCAAGC
AGTTCTCAAAACTCAAGGGCGTGGGGCATGGTGACAGGTGTGACAAGGATGGAGAGGCAGCATTCAAAGAGTGACCCACACCCTTCACTACGGG
GTTCGAGCACACTCAGGGAGCACCTACTGGCCAGACTCTGTCCTATCTGCCCTCTGTCTTTAGGCCTCTGACCCCTCATCTCACCCCCCCCCCC
CCCCCCCCGAGTCACCAGATGCCTAACTGGATACATTTTCAGACTTTCAGGATCCTGGTGCAAAATGCAGACAGGGCCTCCTCCGTCCAACTCC
GGGCTATGGAGTTAGCTGCAGTCACTCTGGGACACACAGCTCTTCTTCTTTAGAAGACCTCTCAGTCCAGGGGCTCCAAACATGGCAAACAGGG
GTGGTTAGAGGTTCCTTCCTGCCCAGTCTTCAAGTGGGTTCTCCACGTGGTTGTGGCTGTCTTCCTACTCAACAAATGAGAGCACAGTTTCATT
TCTACACCTTCCCTGACTGGCTATTCTGTGCCTCTTCAGAAAAAGCCTAATGGAGATAGTGGGTGAGGGACCATCTGCGTCTGGCTGTGACCAC
CTCCTCTCCAGGTGCCCATCGGGGCTGGTAGGTTAACAGTCTGCTGCAGGAAAACAGCTGACTGATCTCTTTATGCCACTGGATCGTTTTTTTT
CCTAAACCACAGGGGGTCCCTTTCATACCCTGGAGAAACATTCCAGAACATGTTCTAGATTGTCATAGACACTCTTGGTTTTAGACTGATACCA
CCTGAAGCAACTCTCAGAGACTCTGGATGGCACACCTAAAGGGTGGTGAAGGCACAGAGATTTCTGTTGTTGTTATTGTTTTACTTTTTTAACT
TGATATAATTCCCTATGGACTTCTTTAAAATATCCTGTCATCTCATTGCAGTGAAAGTCATTCTTTTGACACCAGGGCTGTGGCTTTCTGGATA
CCCCTGGCCAGTATGTAACACTCAGGATGTCCCGGACAGGAAATCCCAGAGCAAGACTGAACCATCTGGATTTCCTGGCCAGACATTCTCCTAG
TGCAATCCTTAGAATCGTCTGGAAGGCTGGTTACAAGTCCTAGACTCCCGTAACTGTGGGATCTCTGATGCTGCAGGACAGAGCTGACGAATCT
GCATTTCCATCAGGTTATGGTGATGGTGACGCTGTCCAGTCTCACTCTGAGAAGCACTGTCGGGCCCGGCAGCACGAAATGCTCTCAAGGACTT
CAGGAATGGATGTGAGAGGCTCTCTCTCCTTTCTCCTATTCACTGACTTAATATTCAGGGGCTCTCAGGACCAGCTCTGTAACCCCTCTTTGGG
TTACCTTTCCACCTTGGGCTGCAGAGCTTCTTCAACCTCACACTCTCTCTCAGAAGAAGAATCCTGGGCTGGAGACAGGAGTGGGTGGAGCCCT
GGTTTTATGTCAGTTTTTGGAGCAAGGCCCAGGTGGGCTATGCACCTGCCTCACCTGCAGGTGACCTTTTCTGTCTACACAGGTGTTCTAGTGG
CATGACGGAGGAGCTACAGGTGGAGAACTTGATTCGGGTCAGACCCTATTCCAGTGACTCTGCCTCTGGAGTCCTCTGATTGAAGGTCTTGTAT
TTGGGAACCTGGGTCTGTCCCCAGCAGCTCTCGGGGCCAGCAGCTCTCGGGGCCACCATCAACAGCTGGTGGGCTGGCTGAGTGGAGCTGAGGA
CTCTGCATCTTGTGGCCATAGGGCTGAGCCAGCTGTGGGATCTCAGGAGCCGGGCCCCAGGTAAGGCAGGATGTGATGGGGGAGGGAGGGCAAG
CACAAGGCAGACAGCACCCTGGCCATCTGCTGTGCAGTCAGTCCCAGCTCCTGGTTTCTCTTCAGAGCCTCTGGAGGAGGCGACCGTCATTTGT
TTAGGCCTCTGCAGTGTTCAATCACCACACAGCACTCCACACAACCCTCCCTCCATCCCTCCAGCTCCTGTTCTCTCTCTCTGCAGGAAACAGC
TTTTGCCTTCTGCCTCTGATGGAGGCCATGAAAATTAACGGAGCTTGAGGCGAAAGCACGGTGGCAGGAGGGGCTGGGGTTGGACGGCCACCTG
GCCAGGCTGAGCGGGCACAGACAGGGTGAGCTTGAGGCCTGAGGGGCTGGCCTGGCTAGACAATGGGCAACCCAGCAAGGCCACAGGACCACGG
ATGGTGGTCAGCAGGCTGGAGCCAGACTTCACAGTTTGGACTCTGCTGGGATCCTGGGCTGAGGGAGAATCTGAGGATGAAACGGCTTCTTTCT
CTGACCTTAAAAGAGCCAGCTCGTACAGGCATTGTGTCCTCAGAAGGTCAGCTGCATATGCTAGACAAATCAGGACACCCCTTTTTCTTTGCCG
TCCTGACAGTGCTGGGAATCGGTGATCACTATGTCTAGTCTCCTGGCCCCCAGAGACACTGAGAGACTTGCCAAAGGACAACCAGCAAGAGCAA
ATGGCAGGGGGTTCTATACTCTTTGAAGGAGAAAAATTACTTATGTACAAAGTACATAGATATATCTCTTTGTAGAGCAGACATTCACACGGGC
AGGAAGAGGGAGCTGGCTCCAGATCCACAGTTCTCCCAATACCTGATCCACCTCCCTCTTGTCCTTCTGAAGAGGCCATGCATGCATACAATTA
GAGTAGTGGAAACAAGAGTCTCTCTCTCTCTCTCTCTCTCTCTCTCTCTTTCTCTCTCACACACACATACACACACACTCCACATTGTCCAGAT
GATGAGATAGGAAGACTCCATCATCTAAGGGCTACAGAAGACTCTCAGGGAAGGTTCCTGCACTAATGGGAAGCTGACCAGCTGTCATACAACA
TGGAGAAAAACTAGCACTGAAATGTCCCATTAGGAAAGTAAATAGGAGCTCAGTGTGCTGGCACATACCTTTAGCCTCACTAAGCACTGGGGAG
GCAGAGGCAGGGGGATCTCTGGGAGTTCTAAGACAACCTGGTCTATGTGGCAAGTTCCAGGACAGCTAGCAGTATAAAAAAAAGAGAGGAAAGA
GGGAGGGAGGAAGGGAGGGAGGAAATGTAAAGGCCAGCTGGCTCAAATAAAGGACAGAGCAGAAGAGAGAAGCCCAAGATGAACAGCTTGACTC
CCCAAGACGGTCCAATGACTCAGACTTCTCATGAGGACACTCCATGTCAGCTCCTTCTAGCCTCCTCTTCAGCCTCCATGAGAACCCAGGACAG
AAAGAGCCCAGGCCTCACTTCCCTGCTCCACACAGATATAACCAAGCAAGAAGAAACAAACTGAAGAGCTCAGGAGTCTGGTGGGGCTTCGGGA
AGCTCGACTCGACAGGGCAACGGCATTTAACGAATCTCATGTCATCCATCATCTGGGTCCAGCCACTTAGATACCGGGCATTTGATTAAACTAA
TATGGGCGGCAGGGCGCTTCCTACCCTCGCCTACAAATTAGACCGTCACACACGATCCGATTACCTTGACTGCCACATTTATCTTGAGAGAGGG
GCCATCCATCACTCCTCAGTGGCCGACTCTACCCACCCCATGCTAATTCCATCAGGAGGAGAGTCAAGTTTCTCCCAGCCTTGCGTCAGGTGGC
GTCTACTCATTCATTCATCCGACATTGGTTCAACACCTACCCACCTCACAGTACCCAGGAAGAAAATGCAGGCTTTCCCTTCAGTGAGCCCACG
GAAGTGGCATGGCACGAGAAAGGTCTACACTGGTGAAGGCAGAQTGCATCTGGCGTACCTGCCTGTGTCCCACCCTGCCTCCATCTGAGACCAC
CCTGTCCTCAAGTGTCACACTTGGGAAATGGTTCAAGAGCATAGCCCAGCACTGCCATGGAATTTCCCCAACTGGAGAAATGTTCTAGATAGAT
CTATGCTGCCCACAACCAAGAGGCAAGGCCACACGCGGCTGTGGAGTACTTCACCCATGGCCCTGCAAGGGGAGCTGAGCTTAAGTCTCATTTA
AGCTTAATTCACTGAATTCGAGTGTCAATGGCCACCTGCGGTCCCTGCCTCCTGTTCCCTAGCATAGATCCAGCAGCTGCTGTGGAGAGCAAGC
TCGGTCCTCATCGTGCCCAGCGCAGAACATGACCCTAGACAAATGCTCACAAAGCCTGGATCCCTGCACTWTAGGATCTCAGGCCAGAGATGGC
TATTCATCATTTGAAGAGCTTGGAACAGGCACTCCTACTAATGGGTCATTATAATCCAACAAGGTGGGATACTCCTCTACTTAAATCCAAGCTG
TGGTTCAGAGGTAAAGACGGTACAAGCCACACCATGGTAAAGGTGGAACCCTGGTTTGGGTCTCTGTGGCTTTGAGCCCTGTCTTCCCACCAAT
TGGACTCCTGGCACTTTAGGAGAAAGTCAGCAGCACTAGAGGCTACCTGTTCTCTGCAGAGAGCCCACACAGCCAGCCTTCCCATGCCACAGCA
AAGCTGGTGGTGGCTACCTCCCTCTACCCTAGGCCTGCTGTAGCCCTGGCAGTTTAGGACAGAGCTGGAGAAGCCACCATAATGTTTAGTTGTA
CCAGGTACAGATGGGACTCCATCCTGAGGCTTAGCAAGAATTCATGGAAGATATGCTGCCTCTGGCTTCCCAGATACCAGTCAGCCCAGCTCTG
CTATCTGGTGGGTAGGTGGGGCTCAACGTGGCTTTGGTTAACGGGGAAGTCTGGGCCTGACTCTGACCCTCAGGCTGCCCCTGGAGAAAGCCAC
ACTATTCCTTCACAATACTGGAGTCAAACGGATATAAAAGAGCAACTGTTTATTCACCATTGAGAACTGTCTGAGGTCATTCACAGGTCAAGTA
CCAGGCAAAGATCACTGTCTGCCCTCAGGCCTGCAGAGATGGCAACTAGGCACAAGCTATAGAATGTCTGTCTGATGACCTCAATCCCCACAGA
TGGACGGTATGGCCTTGGCCAACCCACCTCATCTGGCAGCTTTTAGAATCATACACCGGGGCACAAGTAACAAGTATTCTTGAACCCTCCTGTG
AGCATCGGACAAGATGTGCCTGCCAAGCAGTCACTGCCAAGGTGGATTTTGCTCCACCTGGCACAACCTGAGTCTACCAATCACAGGCAGACTG
TTCTTGCTAGGAACTTAAATGCAGTCTGATACCAGGGAGGGAGAGAAAGAGAGAGAGACAGAGACAGAGAGAGACAGAGACACACGGGCACACA
TACAGAACTGGAGATTAATAGTGCACACACACACACACACACACACACACACACATACACACACAGACTTAGACCTAAAGGCATCCCTGCTGTC
AGAGACTTCTCACAAACCACACTAGAAAGGAGCAGGTTTGGACTACTGAGGCTGCCTGCAAGCCAGAATGGCTGCCCTAGATGGGGTATGTATG
GGAGAGGCCTGAGAGTCCACTACTGGGCTAGGGAAAGAGAAGGAAGGATGTGGTGAAGCGGCAGGTCAGAAGAGCCTACACAGAGAAGCTGACC
TGGTGAGTTACAGTTGAAAAGGATGCTGGGAGGACGGGTTACAGGTGAGTAGQTTCCCAGATTGCTCCCTCGCACAAGGTCCACCTCTTTACAC
TGTCCCAGTTTCAAGGACTACTTAGGCACTCACACATTGGTATAAACAAGTTTACAACAAGTTGTGCCGACACAGTCAAGGAGCAGCAGTTGAA
GAGGAGAAAACAGTGTAGAAAAAATGACGGGTAGCTCTTGCAACGCACCCTTAATTTCCAAGAAGGCACCGGGGAGCACCCCATCAGAGACCCT
CTGCTACCAAGACCACCAGGATTCTCACACCCCTCCCCAAAATGCCAACACTCCTTTGTTGCCACTTAAACCAATTTGGGGGTAATTCTGCTGG
CAATTGTCTTCCGATTTATAACACGGCTCTGTGATTGCATCCAGGCTGATGGTTCAGGCCATAATTGTATGGTAATCACTTCTGTTTATGATCG
ATGCGGAAATCACTATTGTTTTCCAGCAGGCTTGTTAAATCCAAGAAGTGCAGGGGTGGGGGAGGGAGGGTTTATGTCTGAGCCCGAGGAAGAT
GAAATTAACTGGTGCAGCCCAATGTGGGGGAAGGTGGTTCAAAGACACCCTTGTCTCAGAAGCACAGGCACAGTGGGTAGCAGGAAGGCAGCAG
ATGAACAAGCTGAGAGAGACACAAGATGCCCCTGCTGAACAGTCTAAGAACTGTCTGGCACTCAGATCCATAGAGATGTCCAGAGGGACGCCAC
AGTCATACAGTAAGAGGGGAGGGTGCTCTTTAGTACATCCATCCTCATGCCTGCATTTATGAGCTACAGAGAGGCAGAGGAAATGCAGATGCCA
GGAAGCAGGTGGGCTACTGGCTGCCCTATGCTTGGAGCCTCAGGCCTTTCAATCCTAGGTCTACATCGTTTCTTCTTGCCCTTGCCCTGAGATC
GCCTACCTGCTGTCTGAAACAAGGTGACAGATCGCACCTGGCGGTCCAGGTGGCAGGAACAGGGCAGTGTTGTGAGATGAGCTGCTGTTTTGGG
GGAATGAGGGGAACTGCCCACCTGGTGGGTAGAAACCTGAACCTCCCAGAGGCCACAGGGAATGGCCCTGGGCTGGATGGGGGAGGAACAATAC
CTTCCAAGGCTGTGGCAGACACCTTAAGGCACACAAGGATAGAACCAGCCAGGCTGGAGCCCAACAGCCATTCGGAGCACGAGCAGGTACCAGC
TGTCATGTACAGCTCAGGCAAACCTGGATGTTGCAGGCTGAGCTTGGCTGCAAGGTAGATGGTTGTATAAATGTACATTCTTCCTCCAAAGCAA
GAGTGGAAGAGACCATGGAACTCTGGACTGAAGGCTGGGATAGGGAGACCCACTAGGTTCCAAAGCCCCCAGGGAAACACTCACTCCTGGACTC
TTCTCTGAGGAGGCTTTTCTCATCCACTGAACCCCATGGCACCAACTGACACCATTCTGACTGCCCCCAGACTTGGTTCTATATATGGCCCTAG
CTCAGGCTAGCTTTGGGCAACTGTCACAGGCAGCTTCCCTTTGGCTCAGCAAGTTTTTACTTGGAAACAAGTGTGCTTACGGAGCTGGAGAGAT
GGCTCAGCAGTTAAGAGCACTGACTGTTCTCCTAAAGGTCCTGAGTTCAAATCCCAGCAACCACATGATGCCTCACAACCATCCGTAGTTGAGA
TCTGACACCCTCTTCTGGTGTGTCTGAGGACAACTACTGTGTACTTATATAAAACAAACAAACAAATACATCTTTTAAAAACAACACACACAGA
CGTATGCTTACACTAGCTGCCTTACCAATGTCACCCTGGGCTCAAGGCCACCCACCTCATTCTATGCTTTTCTTCACAGACTGAGTGCTACGTT
TCTGTGTCCCATGCTGTCACAACAAAAGTAGCCAGGGTCCTGAGATCATAGAATAATTTGAGTTTGTGTATGCGCAGTGTTGGGGCTCAAACTT
GAGCATGCTAGGTATGAATGAGCACTCTACCAGTGAGCCACATCCCAGCTCCTATTTACTTCTCTTAAATTTATTGCAAGCAACTATTTCAGCC
CATTCTTGAATGGAGAGGACCACCCCAGAGCCCACCAGACTCTTGGGGTTCAGACCCTAAATTTGCCCTTGCTTAGTTCTCCACAGCCACCCTG
TCTATCCTGTACCAGAAGGGGTTTGGGGTGTGTGTGTGTTTCAGAGCACATCAGGGCAGGGCAGAGCATGCCAGAAGCCACCAGAAGTAGAATG
AACATTCTTCATTGAACTAAGATTCCATGGAACTTCTAAGATTCATCTCAAGAAAACAGGCCAGTTCTCCCAGGGATTTGGTTTTCTGAAGTAT
GTCAAGCAAGCCATGGTTACTTAAGGTTCAACACAAAAATCAGTTTAATAACAAGATTTTTTAGCTACAATGAAGTTTTCTAAATGCAGAGACA
AAGATCAAGGTGGGAAACATTGTATCTTCTGCTTTTGTTTTCTTTTTCTTTGGAGGTTGGGGGGCATTTGTAAAAGTAGTGTGTGCTTTGCTCT
CTCTCTCTCTCTCTCTCTCTCTCTCTCTCTCTCTCTCTCTGTGTGTGTGTCTGTCTGTTGTCTGTCCGTGTGTGAACAGGCCAGGGCATCCACA
TGGGCCATATTGGATAGAGTGTCATGAGTATCATCTTTTGGGGTCATGGTGTCATTCTCTCCTTAATACATCGGCTCCTAACCAAGTCCCGGAC
TCCAATAGAAATTCTGTTGTCTGGATTTCCAATTCTTTGGGACAGTTTCAGGGAATAGGAGATATGGCTTGCAAGGGTCTCCTGATGGTGTGAG
AGACTCAGTATGAGAAGTGAGAAAGCTAGAGAGCTTTCACTCTTCCTGACAGCCCAAACTCCCTGGCTCCACACGCTCCATTAGTCCATGACAA
TACCTCCTTTTCTGGGGAGAGAGTGCGGCATGGGCAGGAAAGTCCCTTCTGGACTCTGAAGAGCTCTAACAACATCTACTGTGTCAGCTTATCA
TGGAAGCTTTGGAGGTAGCCGGTAACCTCTGACCTCAACCTGCAATCAGGAGCTTGAGTCAGTTTTAAGATAAGGTCACACGGGTGTTGTTCCT
CCTTGTGCCTGGTCGTAAGAGGACTCAAGGTGAGAAGCTGAATTACCATAGAGAGATCCTTCTACCCTAGACGTAACCCAGGTGGCTGTGACTG
GAGGGACTGCACTTTGAGGGCTCAGAGACCAGTCTTTCCAGGGTCCTTTAGTTCTGGCGCCAAGCAGGGAGAGGACCTGACAAGCCCTCTCTGT
TGGATGCCCAGATGCTCTGCTAACAAACTGTGAAAAGGACCTCTGCACAGATAACTGTAATGACGTGCACAGTATCITCCTATTCCTGGAGATG
GCAGAGGTAACTGGGTGCCAAGGAGAAGCCACAGGATTCCCTTCCCTCAGGCCTGAGAGAATCATATACAATCAGATTCCCAAGGATGGAGCAG
GCTAGGAAGTCACCGTGGCTTTCTGCTCCTGTCCTGAGAGCTACCTCTGGGAACAGTACCCTTAGAAATACTCTACTGGAATGGCAGGCCTGCC
TTCCTGTGGGCATGGCTCAGGGTTTGCTAGCACAGGGTAGCTCATCCTAGCCCTTACCCCACGGTATCCACTGACACACCTACATGCTGTATCC
TTTGTGCTAGGCACACTGGTCACACACGAGTGTGCACACACCCACAAGAACACCACCCCAGAGCCTCATCTATGACTCAATTTGGAAATGTCCT
TTACTGGTCTATAAGGACACACACATGGCCCTATACACACACACAAACACAAGTAATGCTTGTGTTATGCATGCAGTCTCGGCAACAGGTCTAA
CCACTTCGCCTCCAGATACGAGTGTTACCCTGCACATCATGTGCTTTGCAGTCCCACTGGCGGGACTTTAAACTCCCAAGAAGGCAGAGGCCCC
ACCCAGAACCTGCTCTTTGTTCATTCACGCATTCAAAAACATACTTGCTTGCATTTCACCTACCCGGATGCTGAACACACAAACCTGGATGAGC
CACAGCTGCCGAGAGTACTGAGCAGTAGCAGCAGGCAGGGTGGGGGGAACTAGGTATGTGTCAGAGTAAGCACCACAGAAAGGGCATGCCGCCT
GGGCCCCCAGCAGAATGTCCGAGACACACCCGGGGCAGGCACAGCTGAGACCGTGGCACACTACTGATGTTTACAAGCTTAAGGTAGTCTGAGT
GGGGGACGGCACAGTTTCAGTTAACAGGACCCAGGACTAGCGAAGCTGGTCTGAACGGGCCTTTCTAGGGGCTGGGTTTCCTTTAGGGAAGAGT
CCATGGGGCACGCCTAAAGGTGGGAGATCCCACAACCTGGGAAGTAACTCCAGAGAAAACGGTCCCAGAGAAAACGGTCCCGGGGCGCCCACTG
GACAAGAGGGATTTGCCTAAGTCACTGAGGTCCTTTCTGGAGAGAGCCCAGTACTAAAAGCTGAGAGGATTACCAGGCCCTGGTCAGCCTGGCC
CTGGCGAGACTTGATTCTAGGTCAGATGGGTGGCTGGAGGGAAACTGGTAGCGGCATTTCTTTTGTATCCCAAACAGTCTGCTTACTGCTCGCT
GGGCCTTTGGGTGGCAGCAGGACCAGGAAAGAGACAGACCCTGTGCTGTGCCCACAAAGGAGGAAGCGGGAGGGTCCTTCTCACGTCCTGAAGA
TGTCTGGGCGCTTCTTCTGCTTCCAGTTACCTCCCTAGTCTCCACTGTGACCTAGTGGAGACCCACTGGTCACAGCCATGTGTGGCCCAGTGGG
TAGGACAGGGATTTTATATGTTGAGTGCTGACTTCTGCCAGGGCATCCCTGGGACTGTCTGGGAACACTGGGGGATGGTGGTTGGAAAAAAAAC
AAAAGCATGCACACCCAAACTATCCTTAAGTCCCTGTAGAGAAACCACAAACCTTTCTCCTGAGGTTTCTCATGGCATGAGGCCTGGTGGACCC
CAGGACAAGCAAGGCACCCAGGTTTCTCAGGTTCAATGGAGGCTGGGAATTAGGGTAGCGCTAAGCAATGGAGCAGTCGCAGAGGAGGACACCA
GAGAGAGGGTTTGTGGCTAGGCTGTGAGTATAGTGCCTGGGTAAGAGGCCTCTCCCTTTGAGGAACAAGAGGGTGGTACCCCTCTAGAGGGCAG
GTATATGCAAGTACAGAGACTTGTTACAGAGCTAGAAGAGTTAATGGTTCATTAGAACTGACCAGGTTCCCACTGAAAAAACCACCCTCTTTAG
GTGTACTGAATAGCAAGAATCACCTCAGTGTAGCTCAGCTCAAAGACGCTCTGCATAAGAAGCCAGGGGCTTCCAGATGTTGATGGGCCACACA
TAGATCCCAGAAGGGAATGCTTGTGTGAAAAATGTGAGGGCCTTAGGAACCCCATGAAGACTCCCACCCAGCTCAAGCCTCAGGAAGGAAATGA
AAGCGGCTGCCAGCACTTGGGTACTTCCTCTCCAGCTGGGACATGAGCTGGGGCTTCCCAGCCCTGGTCAGTCACCAGTGTCAGTCCTGTGGCT
TTACACCAGGACAGCAGACAACAAGAACACCCGCCGCTCTCCCCACACGGTTTCAGACCACCCCCTTACCTCCCCCGGGTGTGGTGGCCATTCA
CAAGGAAGGGATGGGCATTTCTGTAGGGGCACAGCCAAGATTCTGCTAGTAGTACACAACAGGAAACTGAGGCCCAGAGAAGGCTATGGGCAAG
TCGGGTCAAGAGAGAACCCCCACCTCCCGGGCTTGGAACTCCAGGAGAGTCTGAGTTAAGCTGTCATTTACAGGGTAACTACTATATCCCAGAC
ACAGAGCTAAATGCTCCGCAGCCCTGTGATCTACTGCAGCCTGCCAGGCAAGCAGCACAGATGTTTTATCGATCAAGAAACAAGCATGAATTAA
GTGACTTACCCAAAGTCACCCAGGAAACTACAGATGTGGGTTTCATAATTACGTTCCAACATGGCTTGGGAATACCCTGGGTGACCCTGTTTTA
AGAGGCCTACATGCGTCGGGGTGATGGTGGTGATGGCAACGGGGGCCAGGCAGGCAGGTGAGTCTGAGGCCCTGCTGCAGATCCTACCCCCACT
ACAGACCTCATAAGACATCTTCAAGAGACCTCAGTGACTTAGACACCACACTGCCCAACAGAACCTCCTGTCTTTCTGGGCGTTGTTGCTACAC
CATTCAATATGATATCCACCAAGAACTTCAAATGGTGGGATTAAGGAACTGAAAACCCCAAATCATCTCATTTTAACTAATACATCTAAACAGC
TACACACGCTGGGCTGCTCACACTTGAAGATAGCCTGAGCTACAGAGTAAGACTGTCTCAAAACATAATCCCCAAATTAAAAAACAAAAACACA
ATCAAATAGGCACAGGTAGCTAGCTACTGATAACTAGGTGGAGTTGTGTAATAGACCTGTGAAAGGTGGGACCCCAAGAAAGAGAAGGACTCCT
TAAGCTGTATACCACAAAGGCATCTTGACACCCCGAGCTTAGGACAAGAACCTAGGTAAATATGCTCTGTACAGAGTGATTCTAACTGTTTGTT
TGTTTTGGACATGGTCTCTGTAACTGAGGTTAGCATTAAATTCATGGTAACCCCTCTGCTTCTGCAGGATTTCAAGCACTTCCTAGATGACCAC
TCCTGTCATCACCAGCCTGTGTCCTGGGCTGCTCATGTAACACAAAAATGAGGCACCCAGTATTGACATCCCTCATACATAACCCGTCTGCCCT
TACATTCAAGCAAGGTGACACCTGTGGGTATGTGATCACAGGCTAGCATGTCTAACCTTTTGACATCGTGACATAACACTGTCATTACAAAGCT
TACACTTGAAAACTATATAGCTGAATCACACACACACACACACACACACACACACACACACACACACACTGCAGAAATAGCTCTTAATGTTTTA
AGTGGCTATAATTTTGTGTTGGGCTGTATTTATTCTGCTATCTTCAGCAGCCTGTGCTACATGCTGCTGTCTGCTGGGGACCAGAGGTAGCTTC
CTGGGTGGTGGCATTGGGCTGGGTCTAGATCCAGCAGAACTGAGAGATGAGAGAGGGTTCCAACTCCGGTCTTATCTTTTTTTTTTTAGTATAG
AAGGTGTGAAGAGTTACTCTGTATCTGGTGGACCCAGTCCTTTATTTACTGTGTCAATTACCAATAAACAGAAGGCCAAGTGCCTTCTGCTTAG
CTTGAGAGCAGAGGCCATAGACTCAACCTCCAGCATTGCAAAGAATAGAAAAAGAAAGGAAGGGAAGGAGGTAGAATGGAGATGGGGAGGGAGA
AAGAGAAGGAATAAGGAAGGGATTAAAGGAGGAAGGGAAGGAGGGAGGAGAAAAGAAGAAAAGGGGGAGGGAAGGGGGAAGAACAGGAGGAAGG
GAAGGAAGGAATAGCAGGCAGGCCCTGGGGGCTGCTGCTTCTTTTCCCTTTGAATGATGCTCCCACCCACCACCACCATTTTTCAAAGTCCTCC
TAAGGATAAGTAGAGATGGAACCAGATGTTGTAGAATGTTCTCTGTCTATGGCTATTCCACTAATCAGAGACAAACTCTTGGGAGACAGACAAG
GTCCTGTAAATCATGAGATGCCTCAGGCTCTGACCCTGGGGACAAGGATTAAATCCTGGGGCAACAAATAGGATGTGAGAAGCCCTGGGGGGGG
GGGTGTTCTCTTTAACCTGTCCCCATAGCCCCAGTCTTAGGAAAATGTCCCCATTGCAGTAAAACTTCCCAGACCTGCTTGGGACCCAAGGAGC
TACAGGGCCACAATGTCTATTTTCCCTCTCCTCCCTCTCTCCAGGAGAGCTGCCTGCGGAGATCTGGACAAGTTCCAGCAAGTTCCCAGAGCTC
TGGGGGATGTGGAGCACTGCCTTTGACCCCTACCATGACAACTGCAAAACAGGAGCTCTGTGACCAGAGAGGGCTTTCGTTTTGTCAGCAGAAG
CAAGGGTATCCCCGCTGCAGGATGCTAACCTCCCCTCTTCCACGGCAGAATTACAACACCTTTGCTTCTGGTAACTGTAGTTCTAGGAGCCTCA
GTGGCTCCAAAGTCTTCAACAGCTCTCCCACTGCCCTAGGGAATCAAACCTGGGAGGGAGGGAGGGTTTCAGCCATGGGGCACTTAGCCGCCAC
TGGAGGTTCTGGTCCGCTGACCTCCCATCCAGTACCACACCTCAAGCCTCACTCACTCACATGACATTTATTATCCCGCTCACTCATTTCTGGG
AAACTTCAGGCTGATCCAGGCCCAAAGTCACCATTTCCTGCTCCCTTCCCCAGCTCCACCGTACAGACGAGATAGAGCAGCCCTATCAGAGCCC
TCAGCCCCACCCTCACCCCCACCCCAAGGTGACTTGTCAAAGGTTACATGTACATGTTGCATGCAGTACAGATACTATTACTGGAGGGCTGGGC
CAGAGGAATTGTGTTTATTCTGAACAGTGTCAGAGTTTGTGGGGAGCGCATCCCAGCTGAAATCAATCAGAGGGGCCTCGCTGCAGTAAAATGT
CATCAGAGGCTGGACCAGCCCCTCAGGCAGGCCGTTGCCCTAGGCTAGGCCTCACCTGGAGGCAGGGCTGCAGAAGAAGCCACACGATTATGCA
GGTCTAAGAGATGAGAAGCCCAGAGTGGGCTTCTAGTGGAAGTCAGGGACACAGCTAGGCTGGCTACGGTGAGAGGAAAATAACCTGGACCCCC
TGCCCCAGGATTCTCATCGCGTGGACAGGCAAGGCTTGGCTCCCGGCATCCTCTGGGAGGGGTCCATTGGCAGAGAGACTCTGAAGGAAAGGCA
GCACGGTACCCTGTAAGGGTCTTGTTAACAGGGCTGTAAACTGAGATTGCAAACAAAGCCCCTCTCGGCCTTCAATATCCCCCCTCCGCCCCCC
CTAGATGGGTCTGGAGGACTCCCTTTCGGGTATGCAGAAAGCCACCCCTTTAGATAGCCCAGGGTCTAGCCAGGAAGAGACTACACAGGGACAG
AAATGCAGGTCACTAGTGCTGCCTGGCTTGAGTAGAGGATCAGGGAAGAAGGCGGCAGGGCTCAGTTCAATTGTTAGCTTCCAGCTCTCAGTGA
GGGGCCTGTAGAAGACCAGGTGAGGGAAGAGACTTGCTCCATCCCACACCTGCCCTGCTGTGACCTTGGTCTCTCCTCCCCAGGTAATGGCTGT
TGTCTGTCTGTCCTTCCAGGCCTAGACGTACTGCTCAGGAGCTCCCTAGGAAGCTGGATGTGCTCTGAGGGTGAGGGCCTTGTTTTTCCCCGAG
CGAGCTCCCTTGGGAGGTGGCTGCTGTTTGAAGGCCCGGGAGAGGTTCCCAGGCAGTTGGGGCTTCTGGGGAGCGAGTGCCAGAGCAAGCATAG
CCCAGTGGGTATGCTTCGATACCAGGGATAGTGGGCCCTGGAGGAGGACAGCAAGGGGGTGTGGGGCATGGGGCTGTACCATTTGCCAGGTATC
CAGACCTCCCTTTACCCCTGGGGCTCTCTCTGCTGGTCCTTTCCTTCTTTCTCTGGAGTGAAGCAAGCAACCAGGCTACCTGCCCAGGGATGAG
TCTCTTTCCTTGTGGAGAAGTTGGCTTCTCCTGAGGGACCCAAGCCACATCCAGCAAGGTAAACACCTTCCAGCTCCAGAGTACCTTTCCCTCC
CCTTCAGCCCTGCCAGTTCAGAACTGCCTGTTCTGAGGACCGTCTGATCGATACATGACTTCACAGAGCCTGACGCCTTGCCGGCACCATGGCG
GTCCTCAGGGAGGTCGCCCCTGGACTGCTGCCCATACATGCCCCCTGGCTGACCTCTGTGGCACAGCAGAGTGCTGGCAACTAGCCCTCCGGCA
AAATTTAACCCCAGTATTAACTGCAAACACTGGCTAGTCTATGCCTGCCCCACACAGAGGCTGCCATGACTCAAAACTGTTAAGCATACGGAAA
ACCTAATCTAACAGGCTTATGCTGACATGAGGTGGGCTCACCTCGAGCTTTCACCCAGCACTGTCTACTGGAACTTTCTGCCACTAGAAGACAG
CTGTTTTATAGTCCAAGCTGTCCAGTAGGGTAGCCGCTAGCCTGTGCAGCTATTGAGCATAAGAAACCAATTCCAGCTAGGGATGTAGCTTGGT
TGATACACTGCTTATCTTATGTGCAAACAGCACTGGCTTAAGTACCGGTCACAAATCAAACTGGGCTCGGTGACACATGCCTACCATCCCAACA
CCTGGGAGGAAAACTAAAAGAACTAAGCGAGTATTACTTTTATTGTTAAATCAGTTTAAACTTAGACGCAGCCCTATGTGGTTAGAAGCCACCA
TATTGGATGGTAGTTTTCCACTGAGATCACCAGCCATCCCTCAATGGGGGTGACCTAGCAAGGGCCACTCAGAAAGACCAACGCAGGACGCTGG
TTAACTACACCAGGAACTCAGTTTAGACCTCCTAGGATCTGCATATGGTTGGGTGGTGAGTAGGAGCTCAATCCCTGTGGACAGAGATCTAGAA
GTGCTTTTTAATCCTGCCGTAAGGTTCTTCTACACCTTCACACACTTGCTGAACAGTCACTGAGCATCCACTCATTGACAAGCTTTGTGGTGGG
CAGTGGGGTGCATGGTAGTAAGGAACACATTGCTTTCCTCCGTTCCCTACTGGGCTGAGCTGGACTAATCCCCCCCCCCCCAAAAAAAAAAACA
CACCGGGAGCCTTCTTTCTGGAGTGTGTGGGGCTGTGAGGGGAAGTGTTCACGGTGGAGGGAAGCTGTGCAGGGGAGAGTAAGAGCAAGCAGCC
TGAGGAAGCAAGTCTCAAACTCTGTGCAGGCGGTTGTTTCCGGTACAGACTCAGATTCTCACCAAAGCTTCGCCGTTTGGTGGTTTCTCACTAA
GGAAGTTTGGGGTAAAAATAGCCCTGAAGGGACTTGATTCCAGCTACTTACAGGGTAGTCACTGCCTGGTTAAGATATATATATATATATATAT
ATATATATATATATATATATATCCTTGGTCTGATTTCTAGCCCACACGTAAGGATGGACTGTTGAGAAACACTGGTACCAATACTTTGGCAGCT
GCTTCACAAAACCTCCCTCTTAGAAAATCACCTGCTATGCCCAGGCTCACTGGTAGTTAAGCCTAATGGTCAAGTGCACGCTATGCCTCGGCCA
CACTGTAAGACCTGATGCTCTCCCAAAGCCCGTCTAAGGAACCCTATCTGCCCTCCTAGTCTTACTGCAGCTCGCTTCCAGGTGAACCAAGAAC
TGGGACTTATCTATGCACTGGTTACCAACACTAAAGCCAAATGCTGGGGACCTCTGGCAGCTGCTTCTTGCTTAGCCCTTGCAGTGTTCTGAGA
ACCTCTCTACAAGCTCTTCCTCACAGCCGGATACAGCCTGATGCCAGTCACCCCCCACCATCACCAGCAGCAGCAGGAGCAACAGCAGCAGGAG
CGGCAGCAGCCGCCTTTCCACTAAACACTTGTCAGAACTCTGCTGTGCACCCATCACCTCTGGGAAGGGAGCCCTAGAGCCATACAAGCCTCCA
GGTCTCTCCAGCATCAAGGATTCATTCTCACAAAGGAGTAGCTTTAAGTTAAACCCCTTAGCCACTGCCTCTAACCAAAAGGGTCTGGGCTAGC
TCTGATTTTTTGCAAAGGGAAGCCTACCAGGGATGAAGCCTTGTAAAATGTCTGCTGGGGAACCAGCACTTCACCTTTAGGAAAGAGGCGTGGG
GATCAATCTGTCTGGTCCCCCACCCACCCTCGCATCCAGAGGAGGAACCAAGGTCTCCTCTGTGGGTGAACAAAATGGGCCTGGTGTACACGCT
GAAGCCACTCTTACGGCCAAGCCTCAGGCTGACACCATGCAGCACACGGTGCTACCCACAATGCACTGGCTCTCCTCTGTCACACACATGCACA
CACACTCACCCCCCTCCCCGCCCCACGAGCCTGGCGCCCGCACCCCCGCCCCGCAGCCCGCACTCACTCGCGTGCGCAGCCCGCCGGCCCAGCC
ACTTCACTGCGAGGGACTGGTGACTAGGGATCTGAGGCACCTCACATTTCAAACTTGCTCGCAGGGGTGCTGCTGGTGCACGCCGCCGCCAGAG
CGAACGCACGACCCGACGAGTGGAGCTCCAGCCTCCGGGCGGGGGCGGCGACCAGGCCCCTCTCCCTCCCCCCGCCTCGGCAATTAATTGGAAG
CAACACTTGTGAGAGTGGGCTCCTGGAGCGCTGTCGCCCGAGGGGGCCTCAGGGAAGGCGCTGGCGGCGGTTGACTCCGGGTGCCTCCCGCCCC
CGCCAGCGAATCTTCCCAAGAGGTCATGCTAATGAGGGTCCGCAGCGGGGCCGGGGTGGCCTCCGCAGGCTAAGGGGCGGGGCAGGCTGGGGAC
ATCCAGTGGCAACTTGGGTTTAAAGGGCTTTCTGAAAGGGTTAGTGGGGACGGAGATCAGCAGCTTGGCATCTTACTCCCCCAGCTTAGGGGGT
CCCTGTAGCCCCTGGGATTCTCGGAACGCCCGGGCCAGGGTAGGTGAACTGGCGCACGTACACTCTGCCTCCCTGACTTCGGGGAGTTCCTGTG
CCCCGCAGCCGGCGGCGGGAGGGAAGCCGGAGGCGGCTGCTGTGCACAGCCCCGCCCCTGCTCCCGGTGCTGCCGTCTTCCTGACACTGCATCC
TGTGGTGGCCGCGGCCTCTTCCTTCCAGCCAGTAGTTGCAGGGGCCCGACCCGTGCTTGCCCCTCCCACTCCTCTGCCCCATAGGCACCCCTGG
AAACCCATTAGCTGTCCAGCCCAAAGAGGTCGGATCCCCTCCCTCCTTTGAGGGTTCTATCCACCTAGATCACTCACTAGGGAGAAACTATTTT
ATAAACTCCCCCCAAAACCCGCCCACTCACCAGTGAAAGTTGTTTGAATGAGCTCCTAACTGCCTCAATTAGTCGAGGAGGAAACGCTGGTCAC
TCACCCTCAAGAAAGACAGCCAAGAGCGGCTTTAAATTCTCTGCATCTGCACACTGATCTCTCCAGCTTGGGGACTTGACCCCCTTTGCCTTGG
AGGCTCCTTCTTGTGAGCCTCCTTCACACGCCCCTCCAGTATGAGCTCCTTCCTGACTGGCCCCTCAGCGTTTCTGCTTGCCTCGCTGCGGGCC
ACAGAAGTGGCAAGACAAATCACTTATCTAGACACTGGGCTCTTACTCCTTGTTTGACAGAAGTCGTTAGCAGGCTCTAATCCCACCCACTGCC
ACTTGAAATATCACTTTTGGGATGGCATTTGGAAATGCAGTTGTCATGGGCTTTCCCCTCCCAGGAGCTGGCTAAATGGATTAATTTCTACTTC
ATAACTGGATTTTTCTTTTTGCCCAAGAACCAGCTTCTGCAAAGGAAATTAATTGGCCTCTCCTTCCTGGAGCAACCTCCAGGCAGTGCTGAGG
GACAGACTGACTGCATTCTGCTGTGCACACGTTTCAGGTGGGGTTCACTCTCATCTCCTGTAGCATAATGGCTTCTTTGAGGACATTCATTTAC
CCACTGGAATATTTACTGACACCCTGCCCCTCCAAACATCCACCACAGAGTTTTGAGACTCCAAGAAACTAGATGAAGGCTCTCCCATCCTTCC
CACTGCTCAGCATAGCCAGCTGACAGAGCAGGACACTTTGGCCCACATTTCAGAAGCACACACGTCCCTAAGACCTACTTTCTGGTGTCAGGCT
AACTTAACAATATCAGGGCTCAGTTTCGATTAATCCAAGCACGAAAGCCTCTAAATAACTAGAACCCACACCCCATCCTCCCAAAGTTCTGTAC
AATGACAGTTTACAATTCTCATCTGATTCATAGGGCCTGTTCAGTGGCCAAGGGCTTACACACACAGTAGCAGAAAGAACTAACTTTACAAAGA
TAGTCCTGGGGCAGGAGAGATGGCTCAGTGGTTAAAAGAGGTTTCTTGCTCTTGCAGAGGCCCAGAATTCTCTCCTTGGCACCCATGTGGCAGC
TCACAATCATCTGAAACTCCAGGTTTAGGGAAATTCAACACCCTCTTCTAGTCCGAGGGCACCAGACACACAAGTGGAACACGGACATATAAGC
AAGCAAACATATTCATACATGATATATACATATATAAATAAAATATACATATATTTTGAAATACATATATTAAGAAAGATCATTCCTGCAGGTT
GAGTGAACATTCAGGCTGGAACTGAGCAAAGTTTACACCTTAGGGGCAGGGACTTGGTGGTGGTAATCCTTTAGAAATTATAAAGACAGCTTCC
CGTTGTTTTGCCTCCTCGTCCTCCTACCTCTTAGTTCTCCTCGGGACCCACTTCAGAAGCTCCTGAGTGGGACCCTGAGGATGGAAATAATATT
CAAGATACAGGGTTTTCCAAGGTAACCCAAAAAGTAAATACTCTTCACTAGCATATTTACATACTTAAGAAAACAAATTGTTTTCAAGCACCCA
TTCATCAGTCCAAACTGGCCCCTTTCACTTGCTAATTTCTAGGCTCTCTTCTCTGGAATCAAATCCCTCAAGGCCCTCTTGGTTAGCAGCCTCT
TTTTAGTATTGGAGTCTACAGAAATGCCGGCACTCCACACACTATCTTCGTTAGAAGGGCTTTCCCTGACTCGCTCCAAGTTCCACATCTCTAA
CTTCCAAAGACTTGCTCAAAAGCCCGGGGCCCACAGCCCAAACCCCACCCCACCCCACCCCACCCCGCAGTGCTTGCCTTGATAAGGATCTACA
AAAAGAGGCAGCAGACAAAGGCATCCTGCGACTCCACTTCCAGACGCTCTGCAGCTCTTGGAGTGACCTCGGCAAGCCCCCTCACCCGCCTCCC
TCGGACCCCCCCCCGGGGGGGGGGAATGACTTGGGGGGTAGAAGAAAGGAAGAAGAAAGAAAGGGCGAGCTGTAGCCCCATGCTGTGGCTCCTT
GATGAATGTCTGCCAGGCTCCGGAGACCTGACTAAGGATCAATAGCGGGTGGCTTTTCTCGCGGGGGTCTGAACTCCTGTCGTGGGCCTGAGTG
AAGAGGAGCCGCCAATAAATACAGTGCTGTGGCATGGCTCGCTGGCTCCGGAGTCGGGGATGCTTTATCCTCTGGCAGTGGGGAGCCCGCCCCC
TGAAGGACCCTCTAGAGGAGGGGTGAGAAGGGAGATAATGTAGCCAGGACACCACAGTCCCAAGCTTGGGTCAAAGGACTGACTTCCTTCAGCT
CATGGATGAGCTAGAATCAGTTCCAATAAGACAAGGCACCAAGGCATCTACCTGGGATGCATGTACCAGGGGTCAAAAGTGGGTCACTTCCCAC
TTACACCTTTTTCTTTTTCTTTTTTCTTTTGGTAQTGATATGGTAAGTCTAGGGGCTTCGAAGGCCATCAGGAAGTTAGGAAGAAAGGAAGGAA
GGCTAACTAAGTGGGCAGGCAGGCCTGTCATGCACAAGCCAAACTCTCAAGATGAGGAGAGGGAGAACAACCAGCCTTGATGTGGCTCAGATTC
TGGGCATTGGGTTCTGAGTCCCAGAGAAGTCCCAGACTTCTAACCCAGGCAGTTTGCTGACTCAGAGGGATTGACAAGAATTCTGATGACCTGG
AGAGATGTCACTTCTGTGTACTCAGGCACTAACTGGGAAGGTAGGGTTAACAGGGAGATGGCAAGTGGGTGTGTACCTGGTCGTTGGGAAGCGT
GGAGAAGAACAAGGGGGGACAGAAAGGAATGCTGCTCCGGAGCACCATAAAGAGACTGGGTCACAGGGCACCAGACATACAGACAGCTAAGGAT
CAAATGACAACACTGAGTGTGACTCACCTCCAGGAGTGGGCTCTGCCTCTGGGAACCACCAATCCTGGGGCTTCTGTCTCCTTCTGCATGGTCA
GGACACAAAGGACATCCTTGTAGCACTGTTTCCCCTGCCATTCTCCTTTCTCAGCCCTGATGAGTCTCTCTTACTCTTGAAGAGGGAAGGCACA
GGCAATGTTTGGCTCGTGACCAGTACCGAGCCTAAAAGGTCGCCTATACCAGGGACACAGTGCAGGGCCACCAGGCTGGGCTCCCCTTCTATTC
CTACTGACACCTGGCCATTGAATGCTTGCTGGCTGCTGTATGGGTTTCATCATGTCCTGAGCACTAACTTATAGTATCCTTACCACCCTCTTGT
GTCAGGAGTTATTATGTGCCACATGGAAGGTAAAATGGGGATAGACTGACCAGGTGAAGTTTGCTAAGTATCTAAGCTACTTCTCAGCCTTGAT
ACATCCTCAGCATGCTGCACATTATACTGTCTGGCACGGGATGCCTAGACTCAGCACTGATGGTGCCCACCTGGGCCACACAGCCAGCACAGTG
TGCAGAACTCTTATATAGCTCCTAGAGTATCTCTTTTTAAACTCCAACITGCTATTTGGCCTATTTGCTGTTGACAGAGGCACCTCGACCTGAG
ATGACACAGGAAGGAGACCTCATTACCTCCCTCCTCCCATTATAGTATCCCCTGCCCCTTCCGGGAATCACCCCCCCCCGCCCCACCCCCAGCA
CGGCTCCTCATCCCAGACCCAGGCAGCCTTCTCAGAACCCTGCATTTGGCTCCTGCCCAAATCCTATAATCATTTCATTTTGAAAACAACACCA
GCCCCCTGTCCCTCACCTCCACTCAGCATGTCCGAGAAACCCTACAATTTCCAGGTTATTGGAGATCCAGGAAAGCTTTCTGCAGGGCAGGCAG
TGGGGGAAGGGGGAGTGAGGTGGGCATTTAGGAGCTGGCTCAGAATGGCCCTGTTAGTCTTAGGGAATTGCTTGCCCCCTACCTGCTCAGCTGG
CAAGGGGTCCAGGCTAAGGCTCTATACTTGTGAGGACCCCCTCAGTAGCACTGATGCTGATGGAGAGCCTGGCCTGCAAGGGCAGGAGCTGCCC
CCAGGGCTGGAGGAGAAGCAAGAATCGAGTTGCTGTGTTCCTGGACTCCCTGAGGCACTCAGGAGGCAGAGGTTTAAGAGCCAAACACTAGACA
TGCTAGCTGGGTTTTCAGAATCTACTGGACTTCAAATGAGTGAGAACACCCAACAGCAGGTCCTATAGTGAAAAACCCAACTAAGACAGTTGAG
ATAGGTTGAGAAGTCATGGTATTGGGGTAAATTGTATGCTTCCCCAATTGTTGAAGTCCCTAGGACCTGTGAAGGAAACCCTATTTAGAAACAG
CATTGCTGAAGATCAAGTTAAGATGAGGTCATGAGTGTGGGCCCTGGTCCAGCATGGCTGGTGTCCTTGAGAGCATTTGGGACATTTGGATACA
ACAAGCAGACGGTACCCTCTTCAAGGAGGAGTGAGAAGGACAGTGGAGACTCCAGAGCCTGCAGTGCTTTTGTAGCACCAGCTAAGACACTTCT
AGCAGCTTGGAGGAGCCCTCCTTCCCTGGTACCTACATAGGAGCTTGACACGACCCACACTTTCGTTTTGGGTCTTTTGGGCCCCCAAACTATG
AGATAGTTAATGCACACCATTATGGTACATGTGTTGGGCCCTTTATTAGTGGTTGCCCAGAAAACTACCATGAGGTGATCCCTTTGAAATCTAA
GTCACAGCCCATCATGATTCCAGAGGTTCTTGCTGACTCGGTCCTGATTATCGCTTATGATGTGGGTCTCAGCTTCTGTGGCCAGTCACTGCTC
TAAATCCATTTCCTTAGTGCCTCTGTTGACCCTGCTGCAGCCTGACTCCAGAGTTAGCCATCCTGACTTCGCTCCCTCTTGCAGGGTCTCCCTT
CGCTGAAAGTCCTTCCTCAGCCACACTGTTCCAAGCTATAATCCTTCCCACCTCACCCCGTGCTTCTTCTCTATCTTAGCTTTGCCACACTGAT
GTTTCAAGTATTTATTTTGTTTACTTCTGCCTCTCTGGCTAAAAGGCGGGATCTTTGTCAACTGGAATCCTTGTCTGATACACGCTGTTTGAAC
AGCATCTATGTGTCCTTACAAGATAATAAATACTCTTTGTATAAAGGAGACAAATAGCTACCTGAATTAATGAAGGAGCGAAGAAATGAGTGAG
TGTACACTGGCTAATCACAGGCTCCAGAGATCTGCATGTCCTGGCTACCTGTGCCTGCCTATCCAGGGACTAGGTTGCCAGCCTGCTGTCTGGC
AGAACACAAGCTATTTCCCAGGAAGTCTATTTGAACAGGAAGCTCAGACTGGCCAACAAAGGGGCCGGGAAGTAGTGGGTACAAGCCACATGAG
TCAGGCCTCTAATGAATCTTTCCCCATTGCTAGAGTTGCTTTTGAAGGGAGGGGTACAGAGGCAGCTGAGACATTCAAAGCACAGCAGTCCTGG
GAGAGGGGCTGGGGGGAGGGTATTGCTTCTCCCAGGACACAGGCCTGATGAAGGTTTCCAGATGGCTTCAGGCCTAGACCCACATAAGTTCCTT
GAATTTGGAAATGCAATTTCCTTTCTTGCCAGAGCAGTCTGTCTGTCTGTCTGTTGTCTGTCTGCTTGTGTTACTGTACACTTCCCAGTCTGTT
CGAGGCAGCCTGGGGAATAGCACCTACTTTGCACAGGACAAAAGTGAGGTTCAGATAGGGAAAGGACTGGATGTGACAATGCACACCAGCCAGG
TGGGGGACCAGGCAGGGCTGCAACCCAGGCCCCCTAGCAGCTCATCTTTTTTTTTTTCTTTCTTTCTTTTTTTTTTGGTTTTTCAAGACAGGGT
TTCTCTGTGTAGAGAGGCTGTCCTGAATACTCACTTTGTAGACCAGGCTGGCCTTGAACTCAGAAATCCGCCTGCCTCTGTCTCCTGAGAGCTG
GGATTAAAGGCGTGCGCCACCACACAGACAACTCGGCAGGCCCGTGAGCTTTGCCAGGAAACTGCTACCTGGTGCCATCTTCCCCTCTTCTCCC
TGTCTGGGGTGAGAAGGAGCTCTCCGGGCGGGCTGCCTGCTTCTTATCGGTGCCATTTCCCACAGCGGCTTCTTCCTCTCCCCAGTGTGCTCCC
AGATGTCCCCCACCTCCAAGGGTTCTGGACGTGAGCAGACAGGCCATGCAGGAATGTCTGTCTGGGCAAAATCTGAAGGTGGCCGCCAGGGCGG
GGCGCCGGGTAGGAGGCTCGATGTTGAAATCAGGCTCCTTGGAGCTGAGCTCAGCTGAGCAGCAGATGTCTGACAGGGAGAAAGCTCTGTGGGC
CTGCTGGGGGACCAAGGGCAGGTCGCCGTCCTTTTCTGGGCCCTGGTTTCCTTCCATCACCAGGGAAGTCAGCTAGTGTGGATTCTGTACCTTT
CTTTGTGATGAAGGGCACTGGCTTCCTTAAGTCTAGCTGGGTAAGGTCACCATTGGGGTGACCTTGCTCATGCCCTGGGAAGGTGGCATGGGGC
CAGGCAAGAGACTCCCAATACACATCGTGCTGTCAAGACCAAGAAGCTGCCTCTAAAGGAGCCAACATGCCACACTTCTTTGCCTTTCCCTTTC
CCGGAGCTTATTAATGAGGTTCTAATTCTCTTAGACCAGGTACTCCATAAAGGCAGCAAAGCAGCTCTATCTATTTTGTTCACTTCTGCATCCT
TGATGATTTTGTGCCTGGACCAAGACAGGTACCGGACAAATGATTATCGAAAGACTAATGGAGAGCTACCAACTTATTAGTTTACTGGTAGGAT
GTCAGCTCTACCCTTTACTGTCTATATTCTACGACTAATGTCCAATAAACGACACGTATTATTTTTACGATTAAAAATGCATAACCAACTTTCA
TTTTAAACTTAGTAGAATCTAAAGTAAAGACTCCCCTCCCCCCACCTTTTCTGAAAAGTGCTATATACCGTATTCATAACCAGGGTATCAAGCA
TGAAACAATTTCTTAGCACCAACCGACTTTCAGAAGCCTGGCGTATTGGCGGCTCAGGGTGAGGTACGGCCAGTTGAAATGCTGAAGAGCTACC
CTCTCTCTCTCCCAGCATGTTTGGCTTCCACCCCAGTACAGTCTTGTTCCCGTCTCTAAACTGTATTCTCTGCCTCTGGCCCTAGCTTGCTCTC
CCTGGCAAAGGCTTCTAAAAGGCAAAGCTGACCATGTCACTTGTGTGAACATCAACCTTGCTTTCTACAGTAGCCAGGACCAAGTTCCAAGGTC
TAGCATCTTAGACTGCCATTCATGGCCTCAAGTGATGCTGTCCTTGGCTTGGATGGCTTTTGTCTGGTGCATGCTGTGGTGCCTCTGTGTACGG
ACCTGAGTTTGCCTTCTTTATTAATGTTACTGCTCTCCCTACGTGTCTGTTAACATGCTGGGCTGCAGCCACCTGCCAACATGGAGTTGGGAAG
AAACCTGTTGGCCATGCACGAGTGGCAGGGCCGTGGAGGCTCTCAGTCTTCTGTCCACCAACCTCTGGGATCTTCCAGGAGTCGAGGAAGAATA
AGTCTCTCCCAGTATCTCAGGCTGTCAAAGAGGCCTCATTTCTGGTCCACCATGGCATGAGGATGCTATTGTTTGGGTGCTGTGTCCCTCTAGA
AATCTGCTACCTATTAAGAGACTATGAAGAGGTGGGGCCTGTGGGAGGTGAGAACGTGGTGAGCCTATACCCTCGGGAAGGAGATGATTTCCTT
GATGAAGTGGCTTGAAGAAGCCCTGTCACTCCTCCTCTCAGGAAAAGGATGCAGGAAGAAGGTGCCAGCAGATATCAAGGACCCCCCCACCAGG
CCCATATCTGCTAGCATTTTGATCTTGTCCTTGCCAGCCTCTGTGCTGTGAGCTGTAGATTTCTGTTGTCTATAATGCGCTCAGATCGATGTGT
TTTGTGGTAGCATGTTGCGCATCACCTGCTCTCTCCTTCGGTTTACTCAGTGCACGCGCAGTGTGGCAGCCCTGGCTTCCAAGCCACTGCTGCT
CAATGCTCTGGAGCACAGAGAGCCCACACCCACCTTGGGTTCCAGACAGACCTGGGAGCATGGTATGTAATTGAATTTGGCCTAATTAACTCAT
TGAGCTGAGGAACAACTATTACTGACAGAGCCCTGGGTGTCAGCGGAGGCTGGAAATCAGGAGCCAAGAGAGCTCCCACATCCTCAGAACCCGT
AACTCCTCAGATGCTAGGAGCCCCAGAGGTAGGTAGCACTGATCCTGTCACACAAAAGAAGCAGCTCGAGGCTCTGAGTGACTTAGTAGAGATT
GAAGCCCTCATTCTCTGTGGCCAGGGCCTCTCGACCATGCACTCTCCTCTGCCTGGGATGTCATTCTCACACACTTCTCAGAGCAGCTATGCTT
ATGGGGGCATCCAAACAAGGGTTACTTTCTCAGCCGAGCCTTCCTAGGTTCTCAGCCATGAGCTAGCTGCCTTCACTGTCCTTTGCAGAGGCCT
CATGCTGTAGGGGGAGGAGTGCTCAGCTCACAGTGTTCACTGTCCAGCACAGGCTCAGGCACACAGGCCTGGAAGGTTGGGGGAGGCCCTGCTT
CAAGGCCTAGAGCTGGGGGCTTCAGATGAGTCCTCAACTTGAGGTGAGGAAACAAGGCTCAGGGTCCTACATTTGCAAAAAAAAAAAAAAAAAA
AAAAGCTAGAACTTTATGGGTCTGGGCCTGCCTCAGCCTCCGTGCCCTCTCCCTAGGTCAAGGGAAAGATCAGGCTGACTTAAGCAAACACAAG
CCTCTGCGTCCCAAGGTATATGGTATATGGTATATGGTATATGGTATATGGTATATACTATATGGGAGTTCAGTTCAGTCAATCTGAGTTGGTG
GCCCACTTCCATAGGTCAGGAAGCAGCGCTCCTGCCCACTTCAACCTCCTGGCTGTCCCCAGTCTTCTAGGCATCCCTCTTCCTCTTTCAGGGA
CACAGCCTGAGCTGGGACTCAAGTGTGCCTAGGTGTGAGTTCTAGTCCCACCATTTGCTACTTGAGGCATGTCCAGCATGTCACTCACCTCCCT
GAGTCCCAGCTGAAGATTTCTGCAAGGAGTGAGAGGGAGAACGAGTGTGTCATGCTTGCCCAGAGTCCACAGCATGCTGAAGACTCAAAGCATC
TGCTCCCCAGCGCTTAGTCAGTGCACGGGCCGAGCAGTGCCTGTCTAAAGTGCTGGGTACCAGAAGTATCTCAAATTTCTAGGTTCAGGAGATA
GTAAAATATGTGCATATACATATCCTGGGAAGGAAACCCGAGTCTAACAGTAAATTAATTTCCCAATCACAGGCACCTTATGTGCAAGGAGTCT
GAAGGAAAAATTTAACAACACAGTGTACTTACTCTTAAAACTGTATGCAACAGGGGCTTATAGAGATCAGGCTAACCTCAGGGTGGCTGAGGGT
GACACTGTTTTCCCACCTCTACTGTCTGAGTGCTAGCATGGCAGGTGTGTGCCGCCAAGTTTGGTTTTATGAAATGCTGTGGTTCAAAGCCAGG
CAAGCGTTCTACCAACCAAACCACACCCTGGTCCTGCATTGGGTCTTGACTGTGACCTGTCTTACGGAGCTGGGAGTGGAATTTTTCATCCTTG
AGTACTCAACTGCCAGGCACGGTGGGGAAGACCTGTAATCCCAGCCCCTGGGAAGGGGGTATTAGGAGGACCAGGAGGGGTTTGAGGGCAGCCT
GGATTGTATGAAATCTTGGCTTAAGAAACTTTGAAAGCAGACAAAACAATTCCCCTAAAAGACTTGTGATTGTTACCAGGGGCAGGAAGAGCAA
TGGTGACACACATTGTATGGTGTCAGTCTGGACCTTCAAGGGTCAAGGATGCTGAGTAGACCATGTTCCAGTCTGTGCTGGTCTCTTCTCAGAT
CTGACAGGAGATGGGCCAGTGACCCTCACAAACTCTGAACTCTAACACTCACATCAGAGAGCAGGAACAAAAGCAGGAAGCCCAAGGCCATCAC
ATACACTCTGTGTCATCTCCAGCAAGACACTGCTCTTCTGCTCAACCTACCTCTCACCCACGGCCTCCTCTTTCATAAGGGGTTCTTTCTAGTG
GGTGGAGGACAGTATCCTGTACCTACTGTGTCTTGGCAAAGGAAGCACAGAGTACTCATATAACCCCCGGGAGAAGGGGGTCATAGCTATATTA
TATTACATGTTAAATTATATTATACATTATATTAGAGGAAATGCAGAGTGGTAACGGTAGTCTTCCTAAGATTCACAGCAGCAAAGGCTGGGGC
TGTGTGCCCTGGTACAATGTGTGTTTAGTATGTTTGAGGCTCTAGGTTTGAACTTCAGCACCAATAAGTAGATAAATAAAGCCTCAGCTCAAAA
GCCTAGAACAGCAGCCACTGCTATGAGGTCTCCCCCCAAATGGATCATCCTTGTCCCATCCTTGTTACTCAAATTTAAGTCCAATGGCTGCACA
CTGCTCACACAGAATCAACCTCCAACTACACTGGAAGGGTCAGGACACGGCCTCAGATAAGATGGCTGGCAACAAGAGAAATGTACACTTTCTT
GTAGAATCTGAAGTCCTGGTCCCCAATCCCCAGCACACTCTGGAACCTTGATTTGACACCCCATCTTTGGGTAACCTGCTTAGCTTATGAGACC
CCATCTTTAGGTAATCTGCTTAGCTTAGTAACAGGAGTCCCACATAGAGCTAAGGAGTGCTTCCCTTTCAAQGCACGAGTCTGGCACCAAAAGC
TTAAAACTAACTGTGGTCCATTTTGGGCTCCAAATATCCTGGGGCTCACCTCCTTCTTCCATATTCAAAGGGACAGACTCTCATCTCTTCTTTT
TCTCCATTTAGCATAGGAGAGGCAGACACATGGTCTAGCCTCAGAAGGGGAATACTGGAATCTCTCTCCAGGAATCACAAAGTTGCCTCTGTTC
ACATAAGGGGACTCAGGGATGCCTGGGCATCCTCACTCCCTGGCCTCTGGGAGGGAGTGATTCCAAACATCTGGGGCACGTCTGAGAGGCGATG
AGGACCATAAAAAAACCTTGAGGAAACAGGACTGTATGGTAGCTATGCTATGTCTATGGTTCAGGGCATTCAGGAATGGGCAGAGCTAAACAGC
AGTGGGCTTCCCAGGGATAGGTTTAGTCAGAGAACAAGATGTGATGGGGACTTCAAGTTGGGGTACAGCTGAGCCAGCAGGGCCAGGGTGGCAG
AGAGCTGAGATCCCTGGGGGTACAGAGAAAGACAAAGAAGATGGGCAGATTAGGATGGGGGAGGGGCAGAGGTGGAAAAGGAAATAGAGACTAA
AAGAGGGCGGGGAATGGAGGGAGGGAGAGAGAGGAGGGCTCTCAGGAAGGAAGCCTCAGGCCCCAGGAAATGGAGCCTCCCCTGGCAACTCCAA
CCGGAGAGTAGCATACCCTGGAGGATGTGGTCACCTAGTTTTGTCTTGACCTAGCCTGTCTCCAGCCATGTGACCCTGAGCCTTGGACTTTACT
CCTGTGAGCTTCAGTTTCCTCAACCAGAGAAGGAAAGCACTCCATTGCGAGCTGCTAGATTGAAAAGGCAGAGAGGGCTGTGCTTTCCCCACCT
CCACAAGCGTCTCATGCCCTGCCCTTTGGTTCCAACCTAGTCTAGGTTAGCTGCTTCTCGCTGGATGGCTGGCAGGAGCTTCGCCATGCCCCCT
AACTCTATCCTCCCAGAATGTGCCTCTCCTGACACTGTATTCCCCCTAACCACGTCCCTCCCTCACCCCAGTGTGTGTGCTCTTTGAAGGTACA
GACAATCTGGGGGCACACAGGTGCTTACGGTGCAGGGGTGAATTCAGCTTCTTTTCCGCAGCAAGTTTCTGCTGACTATAATTATGAGGCAGTT
TGAAAGGCAGGCTCCTAGTAATTCCTGTTTCCTGCCAGAGACAATAGTGTCTCTTCATTCCAAACAGGTGAGACTGGGTCCTCTGGAATTCCCT
GCAAGGAGTCCAGCCCCTGGGCAGTGTTCCCCAGCTGGCCAGCCTCAACAAGCCACCATGTCCCTGGGAAGGTAATGAGGCATCTGTGAATGAC
AAACCATCGGCCAGAGAGCTCTGTTGTTGCAGCAAAGGCAACTGCCATAGCCAAGATGGCCCCGCTCCCTTCCAGCCACCTGCCTGGCCGCCAC
CCGGTCCCCAGAGAGGAAATTATCACTATGCAGAGCTTCCACAGCAAAACACAAACACCTGGTTGCCTGGGCAGCACCTTGCCAGGGTGACAAG
ATCCAGATGGTCCCTTGAGAGATAGTAGGGTTGCCACGGAAACAGAATCCCTGCCGCTCTAGCCGAGAGATCTCGATTCTAGATGTACTGGACC
CCAAGCTGAGAGAATTGAGGTGATTTTCATAAAGCTACATCAGTAGGTTTCCAGCTTGAACCACAAAGCAAAGTCCTAGCCAGTGTTGTAAGTT
ATAACTTATAAGTTTATAAGTGATCGGTACTCTGGTGTCCAGTGTTTTGGGAGATGCCCCCTTCCGTGGCATAATTCGTACCTAAAGTCTGGGG
CTTCAGGATAAGGCCGGGGTCACGAAGATGGTGGGGCTAGGCTGCTGCATCAGCTTGACCCTGTAGCCCGAGCTGGACCCCTGCCCTTGGTTTT
CTTCACTTTGTTTTCTCATTCTGAGCTTTAAGTGGCCTATGAACAAGCCTAGCTCAGAAGCCACTGGGACCTGATGAAGATCACCGGAAAGTGG
CAAAAGAGGGCCTATGGTCCTGGGCCAGCATCTAGTGTCTAGGACTTAGTTTCTGAGCATCACTGTGCACATACACAGCTGGGCAGCTCAGAGA
AGGCAGAGGTGCCACGGGGGCACTTCCTCCTAGACACAGTGACTGATCCCAGGGAGGGAGAAACAACAGGTGGAGCCAaGGGAGAGGAGAGAGA
GTGCTGTGCCTGGAGTCAGCTGGCTAAAGCTATACCCTCTTCTGCACTGGCTTCTTCCTCCTGTTCTTCAGTTGTAAGGGACTACCTGTGCTGA
GTGCCACCCCCATCCCCTCCATCATCCCTTGCCTTGTTATCCGTTCGTCCACTTGCCGAGTCCCTGCAATGGGTTGGGTGGGATTGTGATGATC
AGCCCCAAGATTAATAAAAGCAACCTGCACAGCACCAGACAAGAGCTCCATGAATGTCACTGCCTCCTTGCTTCCAATCCTAAGTTCTGAGGAT
GTTGTGGGACACCTGTGGACACAGCTCTGCCTCCCACACCAGGCTCAGCCTCCTGGTCATGAAGAGAGGAGATACACACTGAACTTCATGGAAA
TCCGTAGCCCAGGCCTCTCTGGACTTGGTGTCATAGGGAGTGCCCACTCTTATGCCTGGTATTTAGTCCCCCTGACCTCCAGATGGGTGAAGCT
AGCATTAACTTGCAACCGATGCAAACAGCTCCTTTGCTCTTCAGCCCCCAGCTTTGTGTTCCCCCTAAGGATGCTAGGTACCCCAGGAGACTGG
ATTTCAGCATGCTGGGTAAATTTGCTAACACATAGCCATCACCAAACAATAAGGCTTTGTTCTAAGCATCCTGTGCAGACTGATCAGCTCAAAA
CCTATGACCTCAAATACACACAAAGGTTGGTGACTTTTCCAAGGTTACACAGTTAGTAAGTAGGGGGCTGCGTGCCATTCAGAGCCAGGCGGAC
TGCTTCTGAGCCTGCACGTTTAACTGTTTCTCTTCTCTCTGCACTAGTTTAGTAATTGTTAGAGTTCTTGACGTCAATTAGGGCCAGCCCACAC
TCTCTGTCCCTCACCCTTTGAGTATCAAAGGACCCCAAAGATGGGAAGGTGTCTAACTTACAGCAGAGACAAAGTGCAGGCCACATGCATAGCT
TGAGTCATTAGCATTTTTTATTGTWTATTATTTGTTTGTGTAGCCCAGGTTGGCCTTGAACTTATTATCCTCCAGCTGGCCCATATTCCTTGTT
CTTTACATTTATGGTATTTCTTTGTGTCTATGTGAGGGTGTGTACACACATACATGTGGGGGTCAGAGGACGACATGCAGCAGTTGGTTTTCTC
CATGTACCATGTCGGCTCCAGGGATGGAACTCAGGTTGCTAGCCTTAGTGACAGGTACCTTTATCCACTCAGTCATCATGCCTTATAACCACTG
TCCGCTCCCTCGCCTCCAGACCTGAGTGTGTGTGTGTCTGTCTGTCTGTCTGTCTGTCTGTCTCTAGGATCTCACTATGTAGCCCTGCTGACCT
GAAACTCTCTTCACAGATCAAGCPGGTCTCAAACTCAGAGATTTGCTTCTGCCTCCTGAGAGCAGGGTTATAGATTTATTTTGTTTTTGGTTCT
TCTCAACACGGTGTCACTATATAGATCTGGCTGTCCTGGAACTCACTGTGTACACCAGGCTGGTCTCATGGATCTGCCTGCCTCTGCCTCCTGA
GTGCTGGGATTAACCATGTCTGCCACCATTTTTTTTTTAAATCTCTCTGTCTCTGTCTCTCTTTCTTTCTGTATAATATGTGTGGTTATATGTG
TGTAACCATAACATGCATGTTATAGCACACACATGGAGGTCAGAGAACATTCTTAAATGGTGCTCTTTGCCTTTCACCTCATTTGAGAAACTGC
AATATCACAAGCTTCCAGGTTACTCTTGTCTTGATCTCCCATCTTGTACCAGGAACACTTGGATTACAGACAAGTGTTGCTCTGCTCATCTGTA
TGTGGCTGCTGGGAATTAGAATTCAGGTTTTCATTTTCTCTTAGCAAGTCATACTTCAGGAGCCATCTCCTAAACCTCCCTCCCTTTTTTTTTT
TTTTAAATAAAGATTTATTTATTATTGTACACTGTAGCTGTCCTCAGACACTCCAGAAGAAGGCGCCACCTTCTCCTGATCTTGTTACGGATGG
TTGTGAGCCACCATATGGTTGCTGGGAATTGAACTCATGACCTTCAGAAGAGCAGTCAGTGTTCTTAACCGCTGAGCCATCTCTCCAGCCCCAA
TCTCCCTCCTTTTTAAGACAAGGTTTCATACAACCCCGGCCAACCCCGGCTGGCCTCAATCAAGTATTTACTTTGCTGACACTGGCCTTGAACT
CCTGACCTTCCTGCATTAGCATCCCAAAGGCTGGGATTACAAGTATTCACTACCACACCCAACAGATATTGCTTGACTCACAGATCTGTCTCAC
ATTAGTTAACCTTTCTCAACTGCAACCGGCTCACCTACACCAGGCAATGGGTGCGAACCGCTGGGGTCAGGACAAGGGATGAATGGCGACTGCC
CACTGATCTAAGGTTTGCTGTGTTGTGGCTGAAAGGGGATATAAACTAGATTTTCTCCTAATCCAGATGCCCTTTCCTGACCTACCCTGGCTGT
AAATCTAAACAGGCAAGATAGATAATAACTGCTGATTCTTGTCTGTCTCTTCCAGAAGGTATTTACGAGACTGTCGGCTTGCAACACTCGGAGA
AAAAGTTCCCATACTAACTTGCTCACTGCCTGGCAAGCCCTGGATCCATTTCTCCCTCCTGTCCTGGAGTCCCTGAGAATCCCTCCTCTCGGTG
AGAATGCAAAGCCTGCCTTAAGATCAAAGCCCTGGCTTACTTGATCCTCTCACCTCCTCTTCCTTTTTAGCACTATTGGCTGTGGTTGGCTTTA
TTAAGCTCTACCTCCTTGAGGTACCAGGGAACATCAGAGGGGGGCTGTTTGGCAGGACCTCTGTCCTGAGAACACCTTTGTGTTGGGAAGCTGG
GGGCTGGTATAGAAAGGAGTCACCCATAGGGTGACAGTGCTTTTGGTTCTGCTTTCTCAGAGCAATGTTCTAGCTTCTTGGGCCCCTTACATCC
TGCAGAGAAGCAAAGATGGCCTGGCTGGAGTTCTGGTCTTACTTCTGTCCCTGATACCCAGAGAACTTGTTTTTGCCTTCAAGCCTGCCTTGGG
GATTCTTGGAGTGGGACCTGCAGTCTGGTAAACAGCTGTGGTGGGTGTGGTGCTCCTTCTGTCACCCACAGAGTAGAAGCATTCATTTATTTCA
AACCTCAATAGCTTGGGTGCTCTCTCTCTCTCTGTGTCTCTGTCTCTCTCTCTGCATCTTCAGCCTGTGCTTTCAGTGGGACCGGCACTGGAAT
CCCATCAGTCTTTCCTGTTTGGTATTTTTCATGCTGAGCAGCACCGAGCATCCCCTGACTCTTCCTCCCTCTTGCACCCGTGAAATGGTGCAGA
TGCCTTAGATGGTGGGGGAACTTGAGGCTGGATGGAATAAGTGTCCATCATCCACAGGTCATGACCCACAGGATGGCTCACAGGGCTCCAGGCC
CCCACAAGAAAACTATTGTTCAACGGAGACTCAGGAAGTGGCTTTCATCGTCTTCACAGTTTCTGAATGTGGCAAGGGGTGGCTACTCTACTGG
GATAAGTAGTTCTCTAGGCAGTGGCATCTAGTTCTTTTTTTACAACCCCCTGTGTAGAAGCCAAATGAATAAAGCAAAACTCTCAGTATGCAGT
CATAATTTGTCTAGCCTGGAGCCTGTCCAGGAGATGAGAAGGCTGCAGGGGACTGTGGCAGGGACAGTCCCACGCTAGGCACCGTGAGCGCCTT
ATCTGCTCTCTGCCACGCAGCACACACTGACCTAGCACGTGGCTTTTGCCCCTAGGCAGAGCTACCTGTGTGTTGGAAGGCTAGCCCATCCTCT
CCATCCCTCACCCCAGACAGCCCTCAAGTAAGGATGGAGAAGGGACTAAGCGATGGTGACTAAGTACTTCTTCTGTGGCTCCTCACTGGTGAGA
GTGAGGGGGTGAGTAACAGACCCGCAGGCAGGCTGAGCCCCAGGAGCCCATGGCACATACCATGCCTAACAGCCACACTTCGCTGCGGTCTCTC
CCTCTCACCCATGCTCCTACTTCTCTACAGTTTAGCTGGAGCCTTGACCTCCCCCACCACACACATCCTGGATCCCTTCCCCTCTACTCGCAGG
CGCCAGTGCTTGGACTTCTATGCAGAAAGCTCAAGCCCTGCATCATCTGGTAGGGTCACAGCTGCAGATGTAGACACCTCCTACCGTGGGTGGA
TGCCCTCATCCCCTCCCTATCCCCTTCACCCTCTTGGTTTGGATGGGCTTAGAAAGTAGACACCTGGCTGAGCCCCAGGCAGCCCTGGCCTTGC
ACAAGTCTCTCTCGTAGGGCGGGGAGAGATGAGCTAATTCTGTGAGAGGCAGGAAGAATGGCCTTCTGACAAGAAGAGGCCTGTCCCCTCTGGC
TCTTGGGGCACTATTGCAAGCTGCCTGTATAGAGAAACACCTCAGCCGCCTAGCAGTTGCTTCTGTTCTGAACACCCCTAACAACTCCAATGAG
CTCCGAAGGAATAGAGGGAAGGGTGGGAACACACCCACCTCCTTTGTAGTTGATAGATCTTGTAAGTCAATATTTTGGGACAGTGCTTATTTTC
TGTGCCTAGTGTACTCAAGGGCAGCTTCTGTACCTGACAACTCTAAAACTAACTAACTAACTAACTAACTAACTAACTAACTAACTCTCTCTCT
CTTTCACACACACGCATACACACACACTTACATGCTTTCTCCCTCTCTCTCCTACCTCTCTCTCTGTCTCTCTCTCTCACTCTCTCACACACAC
ATTCCTAAATAAATAAAAACAATCTGCTCAGTCTATATAGTGCTACTTATACAGATGTTTTGAGGGCTGACCACTTAGTGTTGGACGGCCATTC
GGTATGCTCTTCCTTGGTAAGCTGGTTGGTGTTTTTAAAAGCTTAACCTGACGACATCCCAGGCATTCCCACATGGCCCTTGTTTTTAGTGGCC
ACTGTGGCCATGGAGAAACTCAGCCCTTTCCCTGAGTGCAATGCTCTTCCCACATCTGCCTGCTTTCTAACACAGCTCTGCCTCTTCTCCAAAG
TGAGGTTTTAATACCCTGGGGATCATCCAACTGCCCTCCCCCCCAGTTCCTTAGCCTGGGAATGCTGGGCCCACAAGTTTCCTCCCACTGGTCC
TCTTTGTTGAAGAGCAGAGCAAGGTTACAGGAGCCCATGGCACAGCCCTCAGCGAGATGGGAGTTGGGGGAACCAGGGTCATTGCCCCAGTCTT
CCTCGGTCACTCAGGAGGAGGGATAGCTTCCTGGGGCCCAAACTCGACTCGCTTTCAAGCAAATTCTGTCACTAGCACTGAGAACTATAGGGCT
GGAGTCTGTCTGTCCCTCTCAGAGCCTCGATTTCCTCCTCTGTAAATGGGTGTGCTAACAACAGAGCCCCAGTGACCTCTCCCATGGCACAGAG
AACACAGGTTTTGGCCACAACACATATCTGATGGTTAGTCAGTTCCCACAGGCTGGCAGTAAAATCCAGAGCCATTTCCATGGGATCTGGCCTG
GGAGGGGCAGAAGGCCCTGCAGAAGGAAGGAAGCCAAGCCCTCCCCCACAGCCCAGCTCTGCAGAGTTCACATGGGGGTGGGGGTGGGGATGGT
GTCCAGGAGGAAGTCTGTCCCCTGGCCTTGAGAAGGTGCCCCAGCCTGCCAGGCAGGCTGGTGTTTCAAGGACAAGGCCTTGCCTTCCTGCTAT
CTCTGGGGGAACTGGCAGCCACTAACAAATCCCCTCCAATCTGCACCCACCAATCCTGCCTGCTCCCTGGGCAGCCGAGACCCGTTTTTCACGG
GGAGCTGCACACAGCTTTGTCTTAGAGAGACAGCTCTGGCGGCTGCCCCTGAATGGGGCTATTTGTCCTCCATGCTGAAGGGAAGGCAGCTGAA
TGCTCTGTTGGGGTGCGCAGAGGCAGAGAGGGTCGAGAAAGGCTATGGGGGAGGGTGAAGGTAGAGATAGCAGCCACGACACTATGAGAGACCA
GAAGTGCTGTCCACCACGGAGGTGTCTCAGAGTCACCATGCAGCTCGGTGAGTGCAAAAAAAAACCTTGTTTCCACAGCAATGGGACACTGGTA
TATGACCGGCCTTCCCTAGTGTAAAGAGCCCTGGCTGGTAGAAGTTGAAATGACCTGACTGGTTTTGGGTGCCTTTGTCCCATCCCAGCCCCAC
GACAAATTAACTACGGGGCTGCCACACTAGTGCCGTAGAAGGCCCCATTCCAGCCCCTATCCCTGAACTGAGAGCTCTAGAGTGAGACCCAGTA
TCTCTTAAGAGCCTCTTCCACAGATCACAGGCAGAGAGAGGCAAGGCATGTCTCAGTGTCTCCACTAAGACCAGCTGACATCTGACAGGCACAT
GACTCATTAGGGAAACAGAGCAGGCATGCATCTGGGGACTTAAAGCCAGCAAGCGACATTATTTCCACTCCCGTTCTGGGACCTGGGCTGCTGT
ACTGGAAGGTGTGCTTAGAACACAGGTCAGAGGTCAGAGAACCCAGTAGACTTTACCCATCTTGGGGATGCAGACCCAAGGTTTCCATTCAAAG
GATGCAGAACATCTGCGTGATGGTGCTACTCCACGAAGAGCAAGTAAGTGCACATAACTAACAGGCATGTCCTAAAACCACACAGCAGTCCCAC
GTCCACAGCCAGCTCTGGGCAGCCTGGCTGTGTTAAGATTGGCAAACACCCTCTGCCTGTCCCCACTTGAATCATTCCTCTGCCTCCTGTGGTC
CCTTCTCCAAAAGCTTGGCTGGTGCCTGTCAGGATGGCCTGGGTATTCTTGATGCCTCCCACAGTAGCTGGGTGCCTATTCTACCAATAGTCCC
TTTAAACCACCCAAGCATCCAAGCATTGGCTATGCCAGGGAGAAGAAAGAAGACAAAAAAAAAAAAAAAAAAAAAAAGCAGCTGGCACCCCCAC
CTGTGCCCCAGCCCCATTATGGATAGCTTGGGGCGCATCTTCCTTTCCTTTGGTCCCTGGGCTGCTTACCTTCTGGCCTCTGACATTCTCTATT
CTGCTATTTCAACATGTGCTGGCTGCTCAGAGGGTCAGCTGAGGCTACATAGATCTATTCAAGCATAGACACCAGCGGAGGCATGCAGGCAACT
CTTGGGGACCCTCGGACATGGCGACTGCCTGGTTTCTCGCTCCCATGTAGGCTGAACAGAAGGCTGAACCACTGAGTGAGCGCCCAAGAGGCTG
ACATTACAGTACAGCCTGCCAAGTTACTCTGGGCTGGCATGCGGTGGTGGAGGGCGCTGATGGGCATTGCTACAGCCTTGGAGGAGAGCCTGGG
ATGTGTCAGGTGCTGGGGGCTGGGCAGGTGACCTTCTATCTAGGAAGGGGTGGGGGGTGTGGGAGGGTGCAGCCGGCCCCTCAGGAGCCTCAAG
ATGAGGATGCAAGTGCATGTGCGCATGCGTGCTCCTGCTCCCGTGCGCGCAGAGGCTAAAGGCTGCAGCAGAACAAAGAGTTGGGGGCTGGGCA
TCAGAGGGCCCCACGTGGAGGCGGCTGCCTGGGCGGCTTGCTGTAGTGTCTGGGCTGTGCTCAGCTCTGCATTCCGTCTCTGTCTCTCTCATTT
TCGTGCCATACAGTGCTGCCCCCAGGCCTGAGCCTGCTCACCGTCACCACCCTGCTCACTGCCACAACCACGCACACAGCCCAACTCTTTGGTA
GCATTCTTGACAAAGCATCTCCCCTCACAGCATGATCTCATTGCAGGGCCTTCTCAGGCCCCAGCCTGGCCTCCTGACAGGCGAGACAGAAGGC
AGAAGGGTGGGAGAGGCGCCCATAGATGCAGCTAGTTGCGCGTGCACACACTGCTCAGCCATTCCGTAACAGTCATACACCACCCCCAGAACCC
ACACACATCTGTGAACACCTATAGGTCTCTGTGCACTGAAACCCCTCCACCATCACAGGACTCCTCAGGTACATGTGTTCATACTCAACAGTCA
GACATACCAGGCATGAGCATACACGAGTATGCAAACAGACAGACAGACATGTGCATGTGCATGACATACAGCCACACACACAGAGATGCAGACC
TTGTACACACATACCCATACAGTCAGACATACCAGGCATGAACATATATGATCATGCAAACACACATATATACATACATATACACATTACATAC
ATATGTACATAAATACACACATAATACAATGTACACATGCATGGCCACACACAGACCATGTACATACCCACACAGCCAGAAATACCAGGCATGA
ATACACAGGGAGATGGCCTTGAACACACTTATAGCACTCCACACTCACAATGCACACATGCACGATACTTATAGCACTCGACACTCACAATGCA
CACAGGCACGATCCACAGCCACGAGACGCACATACTTCACATAAAGACACACATGCATGCACAGCCATACACATGTAGAGCCACATGTACCCAG
TGTGGGGTTAAAGTCTGAGAGCAAGGAAACTCGGAAAGTCACAGAAAGTCAAGGAAGATAGATATGGCCAAGAATAATGAGGAAGGGACAGTCA
GCAGAGGCCCTCAGGGAGGAAGGACAGCAACACTGCACAGGCAACTAGACAAAGTGGAGTCTAGGCAAATGGGGCAGACGGGGGGGGGGGGAGC
AGCTGGGGTGCCATAGTCTCTGAGCACAGAGTGCCAGGACCAAGGCCTCTTGGGAGACTTAGGAAAGCCATGTGCACCAGAAGGGCAGGTTACA
TCTGGTCATGGACCCACTTATCCACAGAGCTAAGGGGGAGTGATGTGCATTTTGGGCGGAAAGGAGGGGTCAGGAGATGTGTTGCAGACAGACA
CACGGAGAACGACGGCTGGAAAGTGGCATGTGCTGGTGTCCACCATGTCAAGTACCACCTGGGCATCTTTCCTGGCCCACATTGAGAGAGCCAG
CCCTCTGAGTTCCCAGAGGCATCTGTGCACCAGGGCTGCAAGCCTCCAACCACAGGTCTGAGCCAGGCAAGGTGGCTGAGAGATCAACCTTGGG
CCCAGCAAGCCCCTCATCCCTTGAAAGAAACCAGCTGAGAGCCTGCAGCCTATCAAGCAGGGATGCCCTTACACACCTAGGGAAGCTGGCGAGT
CAGGACTGTTGGGTCTGAGGGGTCTGACAGGTCTGCCTCCTAACAAGGGGACATTCCACTTCTGCCAAGGCACAGGCATGCTGGCTTACCAGCT
GACAAAGGAGTTACATGGACAAAGGAACAAACGGACGACACTTTGCCTTAGGCCCAGCACGGGAAGGGGTGCTGTGAACTGTGACCTTGCCCAG
CTGTGACGCAGACAGGGAGAACAAACCTGAGTGTCAGAATCAGAATGCTGAAAATGGCTCTTGGAAAGTGCAGCATCCTGCTGCCGTGCCAAGC
GAGAGGATGCTTGGATCCTAGACAGGAGTCAGCTGAGATGGAGCAGGCAAAGCCCTCAGAGGAGGCTGCAGTCCCGGTTCCAGCTGGATCGTTT
GTTAGCTGTGTGACCGCAGACAACCCCCAAGACTTTTCTGAACTTCAGTGGCCTCATCCGTAAATGTAGATAATATTCTGTGCCTCACAAAGCT
GAGGTTTACTGTGCTTTAGAGACTATTGTATGAGCCATTGGGAGAGGGGTTTAAAAGACCCAGAGGTCAGGTTTCACAGGGCATGAGGCTTCCC
CCTGTACCAAGGACTACTTCTGGCTTCCAGAATGCCCATTCTCCCATTGGCCTTGTCTCTCTTTGCCTACATGATCTTTCTTGACTTTGCCTAT
TATCTACCTATCTATCTACCCATCTATCCATCATCCATCCATCCATCCATCCACCTATTCATCCATCCATCCACCCACCTACCTATCTACCCAT
CTATCTATATCCGTATGGAACCCACGGTCTCATAGATGCTAGGTGTCCTGGTTAGTTTTCTATCAGCTTGACATAGGTGGGGTCATCTGAGAAG
ACAGAACTGCACTTGAGACTATGCCTCCATCAGATTGGCCTCTGGCAAGTCTGCGGGACATTTTCCTGACTAATGATTGATGTGGGAGGGCCCA
GGTCACTGTGGGTGGTGCCAACCCCAGGCAGGTGGTCCTGGATTGCATAAATAAACCAACTGAGCAAGCCACGAGAAGCAAGCCAATAAGCAGC
ATTACTCCATGGTCTTTGCTTCAGTTCCTGCCTCCAGGTTTCTGCCTTGAGTCAGCCCTGACTTTCCTTCCTGATGGAATAGGGTTATCTGTAG
ATGGAAATAAATCCTTTACTCTCCACGTTGCTTTGGATCATGGAAGTTTATCACAGCAATAAAAAACAGCAAAGACACTAGACAAGCATCTTAC
CACTATGCCATGCCCCAGTGGCCTCCTTAGAGACATGACACTGTCCCCTATGTCCACACTGATCTCTGGCCTATAAAGTTATTCTTTGTGCTAG
CAGAGATGGGATGTCATAGCCTGGGATTTTAGCCATGTTCCCATTACCTTTAGGACAGAGAACACAACCTTTAAGACAGGGTCTACATGATTCA
GCTGCCGTTGGCTTCAGCAGCCTCCCTCTGGCCCTGTGCTCTTGCCTCTAGGTTTCAGAACTACCGTCCTTGTTTTAATTCTGACACAAAGCCC
TGTCCTCTTCCCACTCAAGGACCTTTGCACATCGTGCTCCTTTCACCTGTGAAGGAATTGTCACACATTCAACTCTTGCTCAACTTTGGGAAGC
TTCCCTTGACCCTGCTCCGAGCCTTCCTTCACAGCACTGAGTACGGCCTGAACCTGCAGACACTTCAACAAGCGTGTACTCTCCATGTGCATGC
ATGTGCACACCCCCCCCACACACACACATTACAGTCATCTGTCCACTTAGTCCGAAGCAATCAAGAGTGTTAGTCGTCCTGACCCACTCTCCAT
GCTTTACGTGTTTCTAGAACTTACCACGACCCCTGCAAGGCAAGTCTCATTATTCCTACTTTCCCAAGGAAGTCAAACTGAGGCCTAGAGATCA
CAGCTAGCAAGGGCACTCCCAACAGTCAGTTGTCTGTGTTTTTTAAAATCCAAATGGATCCACTGTCTACACATATTTATGAGGCAGGTGTAAA
AGTGGATCATTAAGAAAGCAAACAAACAGAAAAATGGAGATGGGAGTCCATCTTTTTTTGCCCAGGTTGCCCCCAAATCCCAGTAACTCTAATG
TGTTCCCTTCAAAACAGAATGCAGGCTAGGACCTTCCCAGACCTCTGTCAACACGGACTCAGGACCCCTTGCTGTGGCCACGTCTCAATAGACA
ATGTTTTGCAGAAGACATTTTTTTTTTTTTAGAAATGCTAGATTGACAGGTTCTGTTTTGTTTTTGAGTGGGTTTTGAATGAATGAGTAAATGA
ATGAATGAATGAATGAATGAATGAGTGAATGGTGGGCAAGGATCACTGTGCATACTAACTGCAATACTGACCTGACTATGTATAACTCCATGAC
CAGGAAAACCTCTTCTGCTTCCCTAAACCTCACTGTCCTCACTGGTGCAATTGGATAGATAACACAAGGCATTCGCCAAGTGCCCACAGCAGTG
ATGGCACGTGAAGATGCTTGACCTGCCTTTAGGTCAGTGCCTGTGCTGGGGTGTAACAAATCTGAGTGTTGTCCCTCTGCTGGGAGACACCCAG
GGCTGAGCCCTATCTGTAAGCCATAGAGCTGATCACCCTTGGAGGCTTGCACCGCCCCTAGACCAAACAAGGCTCCTCCCACCTTCTGCATCCA
CCTGTCAAGCCCCAGACAGGTCAGTACTTACCAGCAGACTTGGGGGTGAACTTGTCTCGATTTGCGGCGCACACAGCCAAGAGTCTGTAGCCCA
GGTCCAGGTCAAACCCTTGTTGGGCAGCCACACGGCTGACAACCTGACAGCAGGAAGAGGAAATCCAGGGTGAGTTACCATTCCCTCAGAGCTG
AGCCTGAGTAGGTACACATACCCACAACAATAATGAAGCAGGCAAAGGCGCCAGGCTGTGGCAGGGGTCAAAGGCCAGATGGAGTTAGGATTCT
AGCATGTCTCTTTGAATGGCTTGAATGATGACCTAGCTAGGCATTAGCTTGACATGTAGTACCGCCTTCGCTAGAGTGGCTCCAGCTAGGTGCT
TGCATCACCCCATAGCTGCTCACCTTACCTGAGTCCTAGCCCCAGGATGCTTCTCTTCCCTCGGCCTCCTGCCACTATGTCTGGGCTTCTGTTC
CTACCTCAGCAGGTACTCACTACCTCTGCTGCTCCCAGGCACCAGCATATTTTGGGGTATCTCCTGGAGATGTCCTGCATCCCCTACTCTCCTG
CATCAACAGCCATACCCTGAGAAGCAGAGACTTGGGATTTGCTGCTCTTCCAGTAGTTTACAGGCAGAGTTGCCTATAGAATGGCAAAGGCCCC
TGGACCAGGTGGACAGGTGAACCCTGTACTCTGGAGCTCAGCCGGGGGTCTGACTTGGCACTAAGTCCTTAGAGTAAGCCCCTAAATCTTACTC
ATCTGGAGAGATAGGTGCCTTGTTGGGGTACTGTGTGGAACTGGGGTGCCCCAAGGACCAAAGAGAGGCCCGGGAGGCTCCTGCATCTGATTCT
GTCCCAAGAAGACCTATCCCTGGGTGACCAGGTCTGTACTCCCATCTCATTAACAATGGCCAGACAATGCTTCCTTGTTAAGGAGGGAGGAGCT
TAGAAAATGCACTCACCCAGGAGAGCCACTGGGGAAGCCAAGGCTGAGTGGGAGGCTGCTCCCTTCCAGCTGCTGAGGAAGGGAGGCTGAACCC
TCAGGCCTCTGGGCTCTGCCCTACTTGGATGCTGTTGAGGGGCCTCAGGCTGCAGAGGGTTTAGGGCCATCCAGGGCCATCACAATAGCATCCC
ACTTAGGGTGGGGGAGGGCTCACCACAAGGGTTCTATGACTGAAAGGGAAAATCCTTTCAACCAAGCCTGTGAGGCCAGCTCTCCTTTTCCTCT
CCTTGCATCCCATTCTCTCTCTCTCTCTTTGGAAGGGTACCACTGTGCAGCCCAGTCTGGCTTCCCAACTTGTGATCCTTTCGAGTGCTGAGAC
TGCAGATGTACCACCATGCCTCGCTCCATCTCCTGTTTTTAACTGAAGGGACACAGAAGGTCAGGAGTGTAACAGAATGAGGGAGGAGGGAGCA
GAGTGTCCTTTCCCAGTACCTGCCAGACCAGAGCTCTAGACCCCTGTCCTGGAAAACCCCTGCAGGCCCAAAGCCAGCTGGGATGGGAGGAAGC
AGCTAATCTCACTTGGGGATGTTTTCTCGGTTTCAGATGCAAACAGAATCCTTCCATGCTCCCCAGACTCTTTCCCATGGGCTCCCTGGCTCTG
TTTGCTCTCAAGTCTTTAGCCCAGTAGAATGGAGCTTCCTTACCGGCTGCCTCAGCCTTGATCCCTAATAGGATCTGGCCTGTGGAGGTTTCCA
AGTTTCTAGTCAGGGACTCAGGGGAGTGGGGGAAGGTTCTGGACTTTCTGAGCTGCAGCTTAGATGCTGCCTTCGGTGTTTTCCGTTTCGAAGC
ACCCGGGAGCTCAGCCATTGGGGTCACTGTGTCTGACTGGTGCAATGCTAAGGGTTATGCCGGCCCAAGAAACAGTAGGTCTGCTGTGGCCAAG
TCTGGCATGCTTTGGGCACAAGTTGTCTTTTGTTGCTTAAAGAAAGGGGCTTTGCTCAAGATCATATAGCAAAGATATGACAGAGCAAGCAAGC
CAGAGCATGTGACACTACACAGCCTCACGTCCTGCCCAGCCCTTGGCTGAGTCTGAGCCCCCATGAAGGTCTCATAAGGTATGGTCCTTTTGGC
CATTGGTAAGGTATATTCGTAAAATGGGCAGATTATATCTCAGACAGGCCTGTTGTATCCCAGGGTCCTGTAGTTGGCCTAGACTGGATACATG
CTAGAAACCAGCTCATCTAAGGCCCTCCTTCTAGGCATGGCTGGCAGGAACCTAGACGTTAAGAGGGCCGGCTGGCAGGGTCTTTAGTTTCAAT
ACTTCAGAAGGTGAAACTATTCTCCCAGAGACAAGACTTGCTTACAGTATCACAGGGTGCTGCAGCAGCTGCTGCGTCTCTGAAGTTAGGGATG
AATCTGGGGTCTGCATGTGCCCCTCACAGTCATCTCAAGTGACTATCTACTCCCTGGGCCTCAGTTCCTTCACCTGTGAAACGGGGACAAATGT
TCTTATGACTAGTACATTGGACTGAGTCCAGACAGGCCTCCAAGGGTTGATTGAGGTGGCCAGTGGCCTAGAGACTCATATTTGAGTTGACTCT
GGGCAGCTTAGAAGTTGGTGGCAAAACAAACAGACAAACACAAACCAAACCAAACAAAAAAAACCCTAAAAATCAAAACCAAACCAACAACAAC
AAAACCCTCCACACCCATTTGTTCTTTGCAGCTGAGTGGTTTTTAATTTATATCCTCAAAATAAGCCGATCTGGAGGAATTTAGAACTAGCTAC
CAGAGGTCACCGAGGGAACTGTGTCAACCTGCAGGCTTAGCTACCCAAGGCTAGCGAGGGACCCAAGCCTGCAGAGTGTGTGTGTGTGTGTGTG
TGTGTGTGTGTGTGTGTGTGTGTGCGTGTGTGTGCTGGGGAGGGCTTAGCCTCTCCTGCCCTGCAGTCGTCTCTTCCTGCAGGAAACACTGAGG
TAAAGATAGTAACTTCCTACTCTGCCTATGCTGTTCACTTGCAGGACAGAGTCCCACAACTACTTTGCACTTGGACACAACTCTCAGTCTCCCG
CTTCTTTGCTACCAGAGCCTGTCCCTTCCAGCTGACACCTTGGCTCTGTTCTCCTAACAAGGCCTTCTGAGTTCCTATTGTGTTTCATGCCTAC
TGTGGTGGCCCACAGCAGGGAGTAGGTAAATGTCTGCTGAACGACTAAGTGAACGACAAGCAAGACAGGACTAGCCTCTGGTCCATCCACGGTG
GCCAGGGCCAGTGTCCCCCTTGAGTGCATGATGTACGCAGGGCCAAGCCTGGGGCAGTATGCTGGAGTAGCATATTAGTTTGTTTAGCAACATC
CTGTGCATGGCCCCAGCCCCTCTTCCCACTACAGTTACAGGAAACTGGGGTGGGAACTGTGGCCTAGGCAACATCTGGCCCTCTCCATGGGGCT
TAGGCTGGGCCCAGATCCTGCAAAGAGGATTAGACAGCTGGGCGGAGAGGGGAGCTTGGGGACTGAAGGATAAGGAAGACAGGGCCACGCAGGT
GGCTTGAGTGAGACAGATGGGGCCAGCTTCTAATAGACTAACCCTGGGAGGCTCTGTGCCATGTTTTAGATGGACTGGAACACCCCCTTTAGGT
TCACATGGGCCTCAGCTAGAGACCAAGATGCTTTTAAGACTGGGTGGCATGGCAGTCAGTTCTGAGTCTTTGTGTCTTCCTCTGTAAAATAGAA
AGCTTGAAAGACAGGTGGATTCTGGGAGGAGGCACATGCCACAAGCATTTCAGCCTTTCTCAGAACAAGATAGCCTAATGTGGTCATCATGTCT
CCTCTTCTAAACCAGAGCTAAGCTCGGTTCTTTGTCCCTCTCCTGTCTGGAGCACCTTAGGAGACAACAPTCCTGCCTGGCCCTGCATCTCTAG
CCTACATTCCACACTCAGGACACGCAGGCTCCACACAGTGTGATTCACACTCCCTTCTAAGCCCACTACAGGTCTTCTGAGGAACTGTCCTAAT
GCAGGCCACATGGCTGGGGGACATTTAAAAGCTACAGAAGACTCCCAGGTTCTTTCTAGCCTCAAGTTCGGGCAGGTTCAATATCCTGTCTAGT
CTGGACCCAGTTCTCTGGACACCAGGGGCCTTTGCACACGCTGCTTTGTGGCCCAGGGACACTCTGGCTGTGCTCAGCAACCTGGTAAGTTGTG
TCATATGTAGGCGTGGTTTTACATGACTAGCCCCACCTCCCAGCTGGCAAAGAGTCACACCCCCCTCCTGTCACCTGTACTGTTATTCTCAGAC
ATTCCTCCACATTGCTGCTCCTTACTAAGAAATCAAGCTTAAGAAAGTTTTTTTTTTTTTTTTTTTTTTTTTTTTTTATCTTGGGGTTATCCAG
GACTTGGCAGAGGGGACCACAAGTGATAAATGAGGGATCAATCAAGGTCTATTTGTCCAGACGGATGAATGTCACAGGGGCTCTATACCTGCTC
TTGAGTGGACAGACAGACAACCAGAGCGAAGCTCAATAAATGCTCAGCCGATAAAGGAATAAATGGCCTTGGGAAAAGAAGGCTGAGTCCTCCG
AGCTCCCCTGCCTCTGAGGGAGCACTGGTCCCCCACTGTGTGCTTCTCATCAAGTGTGAGGAAGCCACACATCCCATAAAGACGGTCTCCATAT
CAGACCTGGCAGTGGGGTTGTGTGCTCCTGCAGGGCCAGCTTGCAGCATGGGCAGCTGTTCCCCCAGCATTTGGGGCCCACAGAAAGGTATGTG
GGCAGGACTTCTGACCCCAGGGCCATGCTTCCCAGTGTGCTATGAAGGGGAAGTTGAAGACTCACCGTCAGGCAGCCCATGAGGCTCTGCTCAA
AGGGCCTCTGGAAGGCTCGAGGGTCTCCACACCAATCCAGCAGCTCTGTAGCAGCATTGTGGAAGTTGGCGGGGTTCTGTAAGTGCTGTGGAGA
AAAAAAGAGAAAGACTTGAGGCTGCAAGAAGAGGTCAGGGAACAGAGGAGCACAGGACTAGCCAGGAAGGAGACACACAGAAAGAAGGGGCAGA
AGGCTGGTGGGATTCTCCAGCTTTCTCAAACACCCTCTATGCAAAGCCTGGCCCATGGCAGCAGATGAGACCAGCGGACCTATTTCCGGGATGA
GAATCAGGGCTAGTAAGGCCAGAGCCATCAGACATCCAGGTGCTCTGTGGATGCAGGTGGCTGGCGAGGGGTCCCAGGGGGGATTTTAGAAGGT
GGTACCCGTCTCTATACTTGCGGCCAGCAGGCTGTGGCGGTGTCTTCCTAACAACCCACCCCCTCACCCTCTACTCCAGCTCTGGTCCTCCCTG
TGTGTGTCTGAGGAGGAGGACACCCTGGGAAGCGAGACGTGGTCTACATGCTGTCTATAGGATGGTGTGTGTGGCTTAGTGTTCTTAGCTTCAA
AGGCCAGCTCCTCAGGTCTTGATCTGAGCCCTTTTGGAGCCTCTTCCTCATGCCTCACACATAGGAAAGTCAGAATCCTGGGGCTTTCAGAGAT
ACTTGAGGCCCCAGGCTGCCAGCATGGAGGAGTTGACGAGATCTCACTTCATCCTTTATATGGACCCTCAGCTTCCTGGGTCCTTGTCATTTGG
CACTGGGACCAGGAGAGAGAAGACCAGGGCTGTGGGGCAAGTTAGAGCCACAGCTGCGATTTGACCTAGTGGTAGGTATGAGCCCAGAGGTTAT
CATGTAGAAACCAGATTCATGCATACAGGATGCTACCAGCTCCAGGCCCCTTGGTATTGGTAGGATAGCAGGATCTCACTGGCCTAGATGGGCC
CTGTAACTCCTCTGTGGCATGAGTCAATTCACAGCCAGGGCAGAGTGGGTCTTGGGGTTGGCGCCCCAGAATGAGGCTGTGGGAGCCCTTCTGG
GGTTAGGGTTCTGGGGTCACTCACTCTGCCTGTGCCCCTTATTAATCTAGGATGGTCCTTTCACCTCTCCCATCCTCAGTCACCCATCTGTAAG
ATGGGGTTGGGAGACTCCCATGTGGTCATGGGAATTTTGGTTCCTTGTGCTTATCAGAGTATGGTTCGTGCATGGAAACGCTTGCAGCGAGAAG
CAAGTGAGCTCAAAAAAAAACCCCCTCTCCACGAAGAAGAGACAGAGGGAGAAAAAAGGGCAAGCGAGCAGTAACAGCCTGTGTCATGGGGGTC
AGATGACAATTAAACAAGACCAGCTCGGCTTGTGTCGTCCAGGCACAGCACCTGGCAGGTAGCATCGTTCCACCGGCAGCTGCCCTGGGCTCAG
GGTTAGGGTTAGGTCTCAGCCTTTCAGAGGCTCCCCACCTCCTCTGCAGGAGCCCAGCAGCTCTCCCGCTTGGCTCTCTAAAGCTGCAGCTCCT
TGATCACCAAGCCAGGGAGCGGCCGCTGCATACATAGCCACGCCCTTGCCGAGCATGCCTGCTTGGCTTCTCCTTCTCGTCTTCACTTCTAAAT
CTGCCTTTCAGAGCCCCCAGGGAGCACTGGGGCTCTGATGGAGCGTTGCAGCTCAACTCAGCAGCTCCTTCTGGCTTGCGCTCCCCTGACAACC
AGGTGTTTCATTTATTTATGTGATGCCACGACCAATCAGCATGGCCAGCAAGCCGCACATGCACCTACTCTCAGGTGACCATAGCAATGCTCTT
TCGAGACCTGAGCTCCTGAGTGGCCTCTGAGGCTGATCCCCCAAGCC
MOUSE SEQUENCE - mRNA
CGAGCGGCGGCGGCGGCGGGCGCCCGGGAGAGCAAGCTGCCGGCCGGGCGAGCAGCGATCCGGCTGCAGAGCAAGCGAGCAGCGCCCGCGCAGC
GCGCTGACCCTAGCCCCGGCCGGTGAGGAGCCCGAGCCGGAGTCGGAGCCGAGTGGATTTCCGCGCTCTCGCTGCGCTCCTCCCGCCTCCAGCC
TGCCCTACACGGCGGTCGCGCCGGCGCGTCCTCCAGCGGTGGCAGTGGTGCTCGCAGCGCCCGGACTCAGACAGCATAGAATCTACTCAGCATC
TCCTTCGCCACAGCCTCCCTTCCCCAACATCACATTAGCCTTGAGTTGTGATCGACTGGCAGGAGCCCTAGCAGGAGGATGGACCTTGAGTGAA
GTGGAAGAGCTGCTGGACCACGTTCATGGCTCTCGGGTAGGACCCAGCGAAACGGCCAGAATGAATTCTATGGACAGGCACATCCAGCAGACCA
ATGACCGTCTGCAGTGCATCAAGCAGCACTTACAGAACCCCGCCAACTTCCACAATGCTGCTACAGAGCTGCTGGATTGGTGTGGAGACCCTCG
AGCCTTCCACAGGCCCTTTCAGCACAGCCTCATCGGCTGCCTCACGGTTGTCAGCCCTGTGGCTGCCCAACAAGGGTTTGACCTGGACCTGGGC
TACAGACTCTTGGCTGTGTGCGCCGCAAATCGAGACAAGTTCACCCCCAAGTCTGCTGCCCTGCTGTCCTCCTGGTGTGAAGAACTTGGCCGCC
TGCTGCTGCTGCGACATCAGAAGAGCCGCCAGAACGACCCCCCTGGGAAACTGCCCATGCAGCCCCCCCTCAGCTCCATGAGCTCCATGAAACC
CACTCTGTCGCACAGTGATGGGTCATTTCCTTATGACTCTGTCCCTTGGCAGCAGAACACCAATCAGCCTCCCGGCTCCCTCTCCGTGGTCACC
ACGGTGTGGGGAGTGACCAACACATCCCAGAGTCAGGTCCTCGGGAACCCTATGGCCAATGCCAACAACCCTATGAATCCAGGTGGCAACCCCA
TGGCATCAGGCATGAGCACCAGCAACCCCGGCATCAACTCCCCACAGTTCGCAGGGCAACAGCAACAGTTCTCCACCAAAGCTGGCCCTGCACA
GCCCTATATCCAGCCCAACATGTACGGCCGACCTGGCTACCCTGGTAGCGGGGGCTTTGGGGCCAGCCTGAACCCTGCCGGCATGGCAGCTGGC
ATGACACCCTCGGGCATGAGCGGCCCTCCCATGGGCATGAACCAGCCCCGACCACCCGGCATCAGCCCCTTTGGCACACACGGGCAAAGGATGC
CCCAGCAGACCTACCCAGGCCCCCGGCCCCAGTCCCTTCCTATTCAGAGCATAAAGAGGCCATACCCGGGAGAGCCCAACTATGGAAACCAGCA
ATATGGACCAAACAGCCAGTTCCCCACCCAGCCAGGCCAGTACCCTACCCCTAACCCCCCAAGGCCACTCACATCTCCCAACTACCCTGGACAA
AGGATGCCGAGCCAACCCAGCACCGGACAGTACCCACCCCCCACAGTCAACATGGGGCAGTATTACAAGCCAGAACAGTTTAATGGACAGAACA
ACACCTTCTCCTCCGGAAGCAGCTACAGCAGCTACAGCCAAGGGAGCGTCAACAGGTGCAGCTGCTGCAGCAGCGGCCGCTGGATGGGTGAAGT
CAGCAGGAGGCCGAGTATGTGGAGGGATGCCCATGCCTGCTGGGGCACTAGGACCCCCAGGGTAACTGTCAAGCGGAATTTCAGCAGTGTGGCT
GCCTCCTCGGGCAACACAACTCTCAATGGGGAGGATGGCGTGGAGCAGACCGCCATCAAGGTGTCTCTGAAGTGCCCCATCACATTCCGGCGCA
TACAGCTGCCTGCTCGAGGCCACGATTGCAAGCATGTGCAGTGCTTTGACCTGGAGTCATACCTCCAACTGAATTGTGAGCGAGGGACCTGGAG
GTGTCCCGTGTGCAATAAAACTGCTCTGCTCGAGGGTCTGGAAGTGGATCAGTACATGTGGGGGATCCTGAACGCCATCCAACACTCCGAGTTT
GAAGAGGTCACCATTGACCCCACGTGCAGCTGGCGGCCAGTACCCATCAAGTCAGACCTCCACATCAAGGATGACCCCGATGGCATCCCCTCAA
AGCGGTTCAAAACCATGAGCCCCAGCCAGATGATCATGCCCAATGTCATGGAGATGATCGCCGCTCTGGGCCCTGCCCCGTCTCCCTACCCCCT
CCCACCTCCTCCTGGGGGCACCAGCTCCAACGACTACAGCAGCCAAGGAAACAACTACCAGGGTCATGGCAACTTTGACTTCCCCCATGGGAAT
CCCGGAGGGACATCCATGAACGACTTCATGCACGGTCCCCCCCAGCTCTCGCACCCACCGGACATGCCCAACAACATGGCCGCCCTCGAGAAAC
CCCTCAGTCACCCCATGCAGGAACTCCTCCCCGGCAGCCACCCACAGGCCGCTCCCGGCAACCATCCACACAGCGACCTGACCTTTAACCCCTC
CTCAGCCTTAGAGGGTCAGGCCGGAGCACAGGGAGCATCCGACATGCCGGAACCTTCACTGGATCTACTGCCGGAACTCACAAACCCGGATGAG
CTTCTTTCCTACCTGGACCCCCCCGACCTTCCAAGCAATAGCAACGATGACCTCCTGTCTCTCTTTGAGAACAACTGAAGGCCACTCTCCACTG
GGGCCATCCCTCCCCACTCTGCCTCCTACCCCATCTGTCCCACATCCTATTCCTCCCAGGAGCCCATGCTCAGGCCTCCCACACCAGGACCTCA
GGCTGAGAGCTGAGCAGAGCACACTAGGCAGGGAAAGCCATGTGGGTCTCCAGTCCTGTTTGCCTGAATGCTCTGGGATCTATGCCTAGAGCAA
GCCTGAAGACGGCCCCTGGAAGACAAACCCAAGCTGAGGGAATCCAGGCCCAGCAAGAGACATGCCAATGGCGACTGTAGGTCCCTCCATGTGT
GCTGACTCCAGACAACCGACCTTCCAGTGTCCCAAAGTTCTTCCATGTTTTTAAACCTTATCCCCGCCTCTTGGCCCAGAGCCTCCTTCAGATG
ACTGGAACCCGAAAGAAGCCTGTGCTCCGGCCCCCAGGCCAGGGTAGTGGACAAGCCTTGGAGGAGTGGTGACAGTTTGCAGCAGTGAGGAAGG
ACACGCACACACACACACACACACCACAGAAAAGCACAACCCTCTGGGGAGGAGACCATCCCTCAAGGCCGGGGGTCGTGGCTTTGGCCTCTGA
CTTCTAAGCCAAGATGCCCTGTAAACACTCTCTCAGCAGAACAAGGACAAAGTGCCGTGTCTGAGAGAGAGTGCCACACCTTGTAGGGACTGGC
AGGAAACAGGTCAGGGCCTTCCCCAGAGCCAAGACAATGCAGAGGAGGGCTCCTTAACTCAGACCAGGGCCATGCCTTGCGGGGAGCAGAGAGT
GTGTGCACCACACCAAAGGGAG
MOUSE SEQUENCE - CODING
ATGGACAGGCACATCCAGCAGACCAATGACCGTCTGCAGTGCATCAAGCAGCACTTACAGAACCCCGCCAACTTCCACAATGCTGCTACAGAGC
TGCTGGATTGGTGTGGAGACCCTCGAGCCTTCCAGAGGCCCTTTGAGCAGAGCCTCATGGGCTGCCTGACGGTTGTCAGCCGTGTGGCTGCCCA
ACAAGGGTTTGACCTGGACCTGGGCTACAGACTCTTGGCTGTGTGCGCCGCAAATCGAGACAAGTTCACCCCCAAGTCTGCTGCCCTGCTGTCC
TCCTGGTGTGAAGAACTTGGCCGCCTGCTGCTGCTGCGACATCAGAAGAGCCGCCAGAACGACCCCCCTGGGAAACTGCCCATGCAGCCCCCCC
TCAGCTCCATGAGCTCCATGAAACCCACTCTGTCGCACAGTGATGGGTCATTTCCTTATGACTCTGTCCCTTGGCAGCAGAACACCAATCAGCC
TCCCGGCTCCCTCTCCGTGGTCACCACGGTGTGGGGAGTGACCAACACATCCCAGAGTCAGGTCCTCGGGAACCCTATGGCCAATGCCAACAAC
CCTATGAATCCAGGTGGCAACCCCATGGCATCAGGCATGAGCACCAGCAACCCCGGCATCAACTCCCCACAGTTCGCAGGGCAACAGCAACAGT
TCTCCACCAAAGCTGGCCCTGCACAGCCCTATATCCAGCCCAACATGTACGGCCGACCTGGCTACCCTGGTAGCGGGGGCTTTGGGGCCAGTTA
CCCTGGGGGTCCTAGTGCCCCAGCAGGCATGGGCATCCTGAACCCTGCCGGCATGGCAGCTGGCATGACACCCTCGGGCATGAGCGGCCCTCCC
ATGGGCATGAACCAGCCCCGACCACCCGGCATCAGCCCCTTTGGCACACACGGGCAAAGGATGCCCCAGCAGACCTACCCAGGCCCCCGGCCCC
AGTCCCTTCCTATTCAGAGCATAAAGAGGCCATACCCGGGAGAGCCCAACTATGGAAACCAGCAATATGGACCAAACAGCCAGTTCCCCACCCA
GCCAGGCCAGTACCCTACCCCTAACCCCCCAAGGCCACTCACATCTCCCAACTACCCTGGACAAAGGATGCCGAGCCAACCCAGCACCGGACAG
TACCCACCCCCCACAGTCAACATGGGGCAGTATTACAAGCCAGAACAGTTTAATGGACAGAACAACACCTTCTCCTCCGGAAGCAGCTACAGCA
GCTACAGCCAAGGGAGCGTCAACAGGTGCAGCTGCTGCAGCAGCGGCCGCTGGATGGGTGAAGTCAGCAGGAGGCCGAGTATGTGGAGGGATGC
CCATGCCTGCTGGGGCACTAGGACCCCCAGGGTAACTGTCAAGCGGAATTTCAGCAGTGTGGCTGCCTCCTCGGGCAACACAACTCTCAATGGG
GAGGATGGCGTGGAGCAGACCGCCATCAAGGTGTCTCTGAAGTGCCCCATCACATTCCGGCGCATACAGCTGCCTGCTCGAGGCCACGATTGCA
AGCATGTGCAGTGCTTTGACCTGGAGTCATACCTCCAACTGAATTGTGAGCGAGGGACCTGGAGGTGTCCCGTGTGCAATAAAACTGCTCTGCT
CGAGGGTCTGGAAGTGGATCAGTACATGTGGGGGATCCTGAACGCCATCCAACACTCCGAGTTTGAAGAGGTCACCATTGACCCCACGTGCAGC
TGGCGGCCAGTACCCATCAAGTCAGACCTCCACATCAAGGATGACCCCGATGGCATCCCCTCAAAGCGGTTCAAAACCATGAGCCCCAGCCAGA
TGATCATGCCCAATGTCATGGAGATGATCGCCGCTCTGGGCCCTGGCCCGTCTCCCTACCCCCTCCCACCTCCTCCTGGGGGCACCAGCTCCAA
CGACTACAGCAGCCAAGGAAACAACTACCAGGGTCATGGCAACTTTGACTTCCCCCATGGGAATCCCGGAGGGACATCCATGAACGACTTCATG
CACGGTCCCCCCCAGCTCTCGCACCCACCGGACATGCCCAACAACATGGCCGCCCTCGAGAAACCCCTCAGTCACCCCATGCAGGAAACTATGC
CCCACGCTGGCAGTTCTGACCAGCCCCATCCCTCCATACAACAAGGTTTGCACGTACCACACCCCAGCAGCCAGGCAGGGCCTCCATTACATCA
CAGTGGGGCTCCTCCTCCTTCCCAGCCTCCCCGGCAGCCACCACAGGCCGCTCCCGGCAACCATCCACACAGCGACCTGACCTTTAACCCCTCC
TCAGCCTTAGAGGGTCAGGCCGGAGCACAGGGAGCATCCGACATGCCGGAACCTTCACTGGATCTACTGCCGGAACTCACAAACCCGGATGAGC
TTCTTTCCTACCTGGACCCCCCCGACCTTCCAAGCAATAGCAACGATGACCTCCTGTCTCTCTTTGAGAACAACTGA
HUMAN SEQUENCE - GENOMIC
TTGCACACAATCTCTGTCCCCAAGAGGATGGGAGTCCAGACAGGAGCAGGAAATGGGGTTGACATTTCTAATCCTACCAGTTGCTCAAAACCAC
ATTGGAAGTAGTTATCACCATCCCCACTTTACAGATAAGGAAACTGAGGCTCCCAAAGGTGTTGTGAGCTCCCCGAGGGCTCACAGCTCTGAGT
CGCAGGGTTGGGATTGAGGGCCAGGTCATGCGGCTCCAAAGTCCACGCTCTTTCAACAGGCCCACGTTGCCTCTCAGTCTGGCAGGGAGATAAG
CCGTGTACACAAATAGCTGCAATCCCAGGTAGAAAGTGATAAGTGCCAAACAGGGGCGATAAAGGCTCTGGGAGGGGAGGAGACTGCCCACTGC
GGGAGGGGCTAGGGTGGGGGTCAGGACAGGCTTCACAGAGTGGGCAGCATCTCTGCGCAAGCTAAGACATGGGTCGCTGAATCCACAGTATCTA
GCAGTGTCTGACATTGTCACAGGGGCCCAGGAAATATTTGTGAAAGAAAGAGAGGAAGGAAGGGAGGTGAAGAGGGAGAGAGAAAGGCAGAATG
GGAGGGTGGCTATGCTCTGACCCCATTTGGCCCAGGAACTTGCTCCTCTCACAGGAAGACATAGTCAAGAGCTGCCACCAATTAGAGAAGGCCT
TCATGGGGTCCCCATTGTCCTGTGACACCCCAGAGAAGTAGTTCAGGACTTTCAGGCCAGAGTTCCTGACAATCAGATGTTTTGCTCAATACCA
GAAAGAACTTCCCAATGAGTAAAGCTAGACAAGACTTCTCTGCGGGGCAGGGGAAATGGTGTGTTGGAGCCTGTTCATATGGCTCACAAGGGCC
GATTGTTAAATTTTCAGGAACTGCGCGAGCTGGTTGATGGAACCAACCAGCTGTGCCAGCTGGCTCTGGTCGGCAGCTTGAAACTGGCCATGGT
GGGGGTATTTACACCGTAGACATCGGCAAATACTACAAACCAGGTTCTCCCCACTTTGGGGAGCCAGCTGTTAAACATTTACCAGCACACCACT
GGCAGAGGTCTGCTATGTGAGGGAGCAAGCAGCCCGTCCCTGGAAGAGTACAAGCAAAGACAGTCTCCTCCTGGCAGGAACGAGGCTAAGGGGT
TCTTTCTTAGTGAAGGGCTCAGAGCAAATGATCTCAGAAGCCCTGTTCTGGCTCTAAGAGCCTAGGAAAAGTCGGGTTTAAGAAAAAAAACTAA
ATCAGGATAATTCATGCCGGGAAATCATGAACCACTAGACCCAGAGACTGGGAGTAGGGAAGAGACCTGAGAAAATGTGTGAGGGGCCACATGC
TTGGATTTCTCAGGTGGAAAACTCGGTCCAAGGTGACATGGAGAGTCAGGGGCAATGCTAGGACTAGAACTTATGTCTCTGGCTGCCCATCTCA
CGGTTCCTTCTAGGACATATGGGCTGGGCAACTAGGAAGAGGGGGATCTTTCTGCACGAAGTCTGGTGTCTGTCTCAGAATATAAGCTCTGCAG
CAGTGTGGACCAAATCTGTTTTGTTCACTGTTGGAGATCTGGCATACAGCAAGTATTTAATAAATATTCCTTAAAGGAATCGATGTGTCTGAGA
TCCTGTCTCCTGGCAGCATTTGGTCGGGTGGAGGTGGTGGGGGCTGGGAATGTCATCCTCTGACACCGTCACTTCCTGAGTCCCCACTGCAGCT
TAGTGACATGGAATCAAGAGACCAGGGTTCAAATCCCAACTCTGTCACCTCCTTTGACCTTGGCCAAACCATTATGCCTCCTGAACCCTTAGTT
TATTCTTCTACGAAGTGGGTTAGCAATCCCTGCCTGCCTCTTGAGGCTGCTGTGGCACTTGCGTGAGACGTCAAGTACTTTTTACAAACTAAAG
AGCTGTGTCCACAGGAGGAAGGGGGTGCCAGGAGAGCTGGCTGTGGTGGCAGTGCCAACCCAAGGGTCCCTGGTGGACACTGGACAGTGGCCAG
GATGCTGCTCTCTTAGAACTGCTGCTTGGGGTAATCTGGGTCAAACTAGCCAATAGCCTGTGGAGAGGAGGTCTGAGTACCAGACAGCTCACGA
AAGGGGCAGGAAGCAGAGGATGAAATGAAGATGGTAAGGGAGAGTTGGAGAAGTCCTTTTTCCTCCTAAGACCAAAGGCAGAAGGCAAACTAGC
AACTTAAAAAACTGTTTTTGTCTCTGTAGAGCTGTTCATCATGTTCCCCTTTCCCCCACAAGACCTTCACTATTGCAAGAGGCCTTAATGTGTG
AGCTGAGGACTGGCACCTGCCAAGTCAGGTGGGGGTGGGGTGGAAAATCCTCATATGTCCTAGAATCAGACCAGTGTTTCAAACGATTTTGGAA
GAGGGATTTGTGTCGCTCTCCCAGACAAGCATCCCAAGTCCCCTCCTGGTAGGGTGGGAGGACAGGCTGGCGGGGAGGGAGGCCCAGCGCACCC
CAGGCTGTGAGTGGATGAAGAAGGAAAGCAGGGGGTGGGGGTGCTCCTCCAACCCTCCCCCAAGTGCCATCTGATTTTTGCAAAGAGGGTGAAG
GCAGCAGGAGGCTCAGAGCCAGGGCCAGGGAGGCAGGTGGAGAGGGTGTCCGTGGAGGGGCTGCACCGTCAGCCTGGGCCCCCAGTGGAAAGCA
AAATAGAAAGGCCCTGTGTCAGCAGCCAGCCCCCTGGGCAGCCGGGCTCTCGGCTCCATTCTGTTCAGTGCGATTCTCGTTGGCACCAGCTGCA
GTCAGGAACAATGCCTGGATTTCTTTCTTTCTTTCTTTCTTTTTTCCATTTTAATAGAGGGGGGAGAAGGACCAGAGCCACTCATTGAACAGCT
CAGAATTGTGGAGGCATCTGGGACCGTGTGTCCTTGAAAACAGCACAGTCGCAGGGGCCCCACAGCTGGCTGCAAGGTGCAAGAAGGCATGATA
CACTGGGAGCTCCAGGTCCCAGCCCTGCTGCTGTCTCATACTTGCTTGCTGTGTGACCTTAGGCAGGTCCATGCCCCACTCTGGCTTCCCCACA
GCCCCACCAGAGCTTGCCTTCCAGGACCCACTCAATGCCAACTCTTCTTCTTCCCCGAACTCTTAGAATGGCATTGGGTGGCCGGGTGCGGTGG
CTCATGCCTATAATCCCAGCACTTTGGGGGGCCAAGGCGGGTGGATAATGAGGTCAGGAGATGCAGACCATCCTGGTTAACATGGTGAAACCCC
ATCTCTACTAAAAACACAAAAAATTAGCCGGGTATGGTGGCATAGTCCCAGCTACTCGGGAGGCTGAGGCAGGAGAATGGTGTGAACCTGGGAG
GCGGAGCTTGCAGTGAGCCGAGATCTCACCACTGCACACTCCAGCCTGGGCGACAGAGCAAAACTCTGTCTCAAGAAAAAAAAAAAAAAAAAAG
AATGGCATTTCGTGTGGGCAGTCCTTCCTCAGAGACTGTGAGCCCCCAGAGGGCTGCTTCTGGTCATCCTCCATCCTGTGGGCCTCTATACCTC
CCCTGCTCCGGATGGCCTGTCAGCCCCCAAGGCCTTGCACTGGGTGATGGGATGTAGCAGTGAGCCATCCTATGACCTGCAGCCTTGACAGAAG
AGGGCCATGTAACACAATACCTCCCTAGCCTGTGAGGTGTCATACTAGGAAATGAAACTCCCTGTGCCTCAGTTTCCTCATCTGTAAAATGAGA
ATACTAATAGCTCAGAGGATGGGAGGAGTTCATGTGTATGAGCTACTCAATGCCTGGAACATAGCAGGAACTTCACAAGTAATTCCTATGGTTA
TGCTGATGTTGGAGGCTGGGAGAGCAGCATATGGGGGTTAAGACAAGGAAGACGGCACATCTGGTGGAGGTATCAGGGTAGGCTTCCAAGCAAG
TGGTTGCAGATGTGGCCTTGGTGACTGGGAGGGGTGTGTCCCAGGAGGTAGGAAGAGCATGAGCAATGGCACAGAGGTAAGAGTATGTACATAA
TGCTGGCAATTCTGGAAGTCACCCTGCATGGCCACAGGATGAGGTGAGGGCAAGGAAGCATGAGTCTTGAGGCTGGAGGACTGCACAGCCAGAA
GGAGGAGAGATGCTGAGGATCTGAGACCTTCCCCCACCCCTCTCGGGGTGCAGGAGCTGTCAGGAAGGAAGTGGGAGGCATGGTCCTCCTCAGG
TCGCTTGTCCCAGACTTCCTCATCAGTGCCGCATGTACTTGCTCCTCTGGAGGGGCGCTGACGCAGGTGAGGGGACACTGCATGAGGAGATGTG
GCTCTTGCACCCCCTCTTGTCCCCCCACCGAGTTGCTGGGACTACAGGCATGTGCCACCATGCCTGGCTGAGACCTGGCTCTTGTTCCAGCTCT
GGTGCTTCCCAGTGGTGCAGTCAGGGAAAGCCATCACCTCCTTTTGGGGCTCAGCTTAGTCACCCACAAAAGGGCTGTTGTGAGGCTCAGAGGG
TGAGAGGTGCCTCATGAAGGGAGGCGTTAGGGTCATTACCTATTCACAGTCCTAGATAGTCACAGTTCTGAGCAGCTCCCTCCAGGTATTTTCT
TGTCCCTACTCCACTAAATGGCCCCCTCTGAAAGCCCTCCCTAAAGCAGGGCCGCCCTCAAACTCTAAAGTGAACTGTAGCTCAGTCCAAGGGT
TAAAGGCTCAGACCTACAGAATCTTAGGACAATCAGAGGCAAAAGTGCTGGTGGATATCCCACACTCTCTCACTTGGCAGATGGGGAACAGGCA
CAGGGAGAGTTTGTGATGTGCTCAGGCCACACAGCAAGTCAGGCAATGGCGGGCTGGGCACATAGGTGTCCCGGCTCCTGAGGCAGGCTCCCAG
CCTATTCTCTTGTGACTCCCATCTGAAGAAGGGGTTGGTGGTTGAACTGGGTCTCGAACCATAGGGCAGAACTCCATGGTAGAAAGTGAGAAGA
CGGCTGGCTTGAGGGAAGGACATTGGAGAGTCAAGCCTTGGAGGATGGGAAGGTGTGGGCTCAGAGAATGGAGGGGCCATCTGATTGGAAATGA
GGGGGAGGCAGAGCAGGGGAGCCTAATTCGCTGAAGGACATAGATCTTCCCTGCTTGACCCAACCACTCACCTCTTAGAGGCACAGGGCTCAGA
GCTGGGCAGGGCCCAAATGCTTCCTGTATCCACCAAGGGGTGAACTCTAAATGTAATCTCCATTACCTCACTGCCATGGGAGGCACCCTGGTCA
TTCCCTGGCAAGTGGGTGAGAGTCTTTACTTCACCCCAAAGTACCTGGTAGGTTACCTTATCTGCACCATAACCTGGCAGTGAGATGAGGGCAA
GTCCCTATTCTCAACCTCAAGGCCCAGCCCAACCTGGAGCCCAATCTCGGCTCCACCCACTTAGCAGCTCTGAGATCTTTATTGTTTTGTTTCG
TTTTTATGAGACAGGGTCATGCCCTGTTGCCCAGTCTGGAGTGCAGTGGCACAATCATGGCTCACTACAGCCTCAGCCTCCCAGGATCAAGTGA
TCCTCCGACCTCAGCTTCCCAAATAGCTGGGACTACAGGTATGTGCCACCATGCCTGGTTAATTTTTGTATTTTTTGTAGAGGCAAGGTCTTGC
TATGTTGCCCAGGCTGGTCTCGAACTCCTGAGCTCAAGTGATCTACCCTGCCTTGGCCTCCTAAAGTGCACAGATTACAGGCATGAGCCACCAT
GCTCAGCCAGCTATGAGATCTTGAGCAGGTCACTTAACCTCTCTGAGCCTCTTCCCTTAGCAGGGAATGACACCTGGCTTGTGGAGAGAATACA
ACGAATGTTTCCAAAGTGCGTGGCATACAGTAAGTACTCAATGAATGAGATTTTCTTCCCACCTCTCTAGTCTCAATTTTAGATGTTCCCTGGC
TCCCTTAAGTTCCAGAAGTGAGAGGTGGAGAGTGGGGACAGAGAAGGCAGCTCCTGGACAGGTGGGAATCCCCACTGTGTACCCGCCCCCATCC
CAGCCCAGCCTAGGAGGCCATACTCGCCGCACCCTACACAAGTCCTTTGCTTCCCTCAGGGACCTCAAGCCTCCCACCCCCACCATCCTGCTGG
CCTCCAGATTTTCTGCCTCCTGAAACCTGGGCCCAGGCCCCAGCAATGGTCCTCTCCCCAATCTGCTGGTGGCTCCCACAGGACAGGGAAATTA
ACAGCACTTGAGTGTGCTCTGAAGGTTGGTACAACCTGGCGGCCCCACACTGCCCATGCCCTCCTAGTGGAGCCAGGTGACTTCTGGGCCTGCA
GGGGCTGAGCAGGATGCATCTTCCAGCTCTGGGCCACGAGGAAGTGATGGCACTGTCCTCTCTTGTCTCACCAGCAGATCTCCAGGCCCAGTGA
GGCTGAGGCTTTGGGGGTGAGGTTGTTGGACACACAGTTCCCAGTGAGTCCTGATGGCACCATGGACCTTACAGGGGCTCAGAGGGGGAAAGCA
GCTTGCTTGGGGCTACACAGTCTATGGATGAACAGAGCCAGGGCTGAGGGGACAACAGGAAACTCTGTTCATGGGGGCCCGAGTTCCCATCCTG
ACCAATCTTGGGCAAGTCATTCACAGTTGAGTTTCCTCTGAAAGGGGGATGGTAGCGGCACCTAACTTAAGACAATGTGACAGTTCAATCAGGG
AACTGAATTCTCAATACCTAGGAGGACCCACCACAGTACTCACCACATAGTAGACAGTAAATAAGTCATAGCTTCTTCCAAGACCCCAGAGGTA
TCCAATCTACCCCATTACCTCCCTGGAAGATGAACAAAGAGAGCAAGGGCCTCAGAGACCCAAGGACTGGAGCCTAGGGAAGACCCAGGACCCT
GGTGGGCTTTCTGACTTGCTGCCATGCTTGCACAGAAGGGGAGGGTTGAAATGATCCCTGGTTCCATGCTACTAAGTTGAGCCTACTTGGCTCC
TCTGCCCTCTGCAGCCACTTCCTGGGTTTCAGATGTAAATACTGGTGGGAGAGTAGGGGGCTGGGATGTGGGGTAGGGGGTGGAGCTTCTGGAC
AGGAGACTTTGAACATGAGTGGTCACCTCCGAGGTCACCTGCATCCCCCAATTCCTGGAAGTGGGCAGGCCCTGGATACTTATTGAGGAATCTG
GACAGAAAGGTTGAGAGGGCTGGGCGTCCAGACCTAGCTGGTCATTAAGGCAACAAGAACTGGTCTAATTGTGGGCTGGGCTGGATTATCACAT
CCCAAATCCTTAAATACCCAGAAACTCTCTTTTGGGATTTTGGACCTGGGTATGGGGTGGCTGCTGCCACTGCTGCAATGCTGTGGATTGCTGG
GAGTGGGACAGCTGTGGCTGCAGCCACCGTTCTGCAAGCTTAATTCCTACCTTCGGAGGTTTCTCAGGTGGCCCTGGTGTACCTACCTGTGAAC
TTAGGCCACCCACAGGCTGAGGAGGGGCTCAATCACACAGCAATGTGCTGGGACCATGACTCCCTGGTGTCCCTAGAGGTCTGGAAGTGCTTTC
ATACCACCCACCCTCTCCAGGGATGCTCAGCTGCCACCTACAGGGAGGGCGGGGGTGGAAGAGCAGCAGTCATCACAGTCCATGTCCATGTGAC
AGGTAGGAAGACTGAGGCCCCAAAGGAGTGCCTTTTCTTGCACCCAATGGCATAGGGAGGCAGAATTTCCCCTGGGGAAAATAAAATAACGCCC
CCACTCCGCCGCCTGCGCCCCCACACACCCCGTCCCCCACGCCCGGGTGGACCTAAGCAGCAGTTTTCTGAGCTTTCAGGAATTCCAGACTGAG
TTCACTGGCTGTGAGACTAGCGGAGCCTTGAGAATGAATTCAAAGCTGGCAAATTCATTTTGCCTCCTCTCCCTCGGTGGTGGAAGGAGTGATC
TCTCCGGCTCTGAAGGAGTTCAAGCCGAGGTCAGCGAAGCCCTCAGAAGGATGTTCTAAAATGGGGGTGGGGGGTATGTGCTCCGGCCTGCTTC
CCCGGTTGGCCCAAGGTCCGCGCTGATCACCGCCCAAGAGGCTGGTACTTTTCACTGTCTGTGGACATTAAAAAAGCGAGCGGCGGCGGCGGGC
GCCGGGGAGAGCGGGCGGCCGGGCGGCAGGCGGGCGAGCAGCGATCGGGCGGCCGAGCGAGCGAGCAACGCCGGCGCAGCGCGGTGACCCCAGC
CCCAGCCGGCGCGGAGCAGGAGCCGGAGCCGACCGCATCTCGGCGCCCTCGCTGCGCTCCTCCCGGCCCGAGCCTGCCCTACCCGGCGGTGGCG
GCGGCGCGTCCTCCATCGGCGGCAGCGGCGCTCGCAGCGCCCGGTAAGTTTGGGGCCAGAGCCAGGCGCCCGCTGGCTCCGGGGCTACCTCCCG
CTCCCCGGGGACTCTCGGGGTGCAAGCGTGGGGATTGGGTGCTGGGGTTTTTCCCTTAAAGTGTCCCCCGGAGCGGGGCGACCGGCCGGCGGCG
CGGGCTCCGCTCCGCTCTCCTTGGCGGCCGGGGGCCGAGCCCCGGCGGCCGGCCAGCTCCCGGCTGCGCGGAGCCGGCTTGGGCTGCGCGTGGG
GGCCGGGTTTGTCAGGGTGTGCGGGGCGTAAGTGTGGTCGTGCGCGCGCGGACCCGGTGCCCGCCTCCTGCCGGCGCGCCTCCAGCCCTCGCTC
CCTACACCCGGGGGCCGGGCAGGACCGGGAATCGGAGGAGGGAGGGAAGGACCGCCTCGGTCTCCTTCGCCTCCTCTGGGGAGATGCGAAGTTG
CCTACACCCGGGGGCCGGGCAGGACCGGGAATCGGAGGAGGGAGGGAAGGACCGCCTCGGTCTCCTTCGCCTCCTCTGGGGAGATGCGAAGTTG
TGCGCCTGTGTGTGTACAGGAACCGCTGGGGAAAGCGCTGGATTTCAGGATGGCAAGGGGTGGTGGTGTTAACTTGTGCGTCTGAATTTCCAGG
GAAAACATACACTTTTGTAAATGGGAGGTGGGGGCGCGGTTAAGTTGTTTGTGTGGGAATTGCCCGGGGAAAGCTGGCGCGCGGTACAAACGAG
AAACGCGGAGGTGTGTGTGCAGGGTTTTCTCCCAGGGCTTCGCAAGCCAGAGGGGCGCTCAGGCCAGTGCCTGGACGGGGCTGCCCCGGCCAGG
AGGGGAGCCCAGGGGGAAGATGTGCGTGTGTGTGCGCGCGCGCGTTGCAAATACGAAAGTAATTTCCCCGTGTTCTTTCTTCCTCTCCGTCTCC
GGCGGCGGTGGCCCCGGGGGAGTGGGAGGCCAGCCGGGGCGGAGGTTGGGGGGCCGGGGCTGGAGCCGGGGTCGGGGGGGGGAGGCGGGTTCTG
GAAGCTTGCTTCACATTCCTGGCGTCTGGGCTGGGGAAAAAACAGTGGGGAGCGCGGCCTCGGCGTCTAGGCCGCCGGCAGCGGGCGCGCGTCG
CCCCCTCGCTGGGGACATCTGTCCAGGAAGCCGGCCGGGCGCGGATAGGGCGCGGTGCGCAGGGCAGGCTCCCCGCCCTGGAGCGGTGGGGGCG
CGGGCGAGGGGCGGCCGCCTTCCCGGCCATTGTGCGCCAGCAGATTTTCACGGGAGACTGCCCCGGAGCCGCCGGAAAGGCCGGGAGCCGGCTC
AGCCCCGGGTTTAAAAGACACTCCAGGAATGTAAACAACCCGGGCGGGAGGGCGGGCGGACGGGACCGCGCCAGGGCAGCCGGCCCGGGCCCCC
CACCCCTCCCCGGGGACAGCCGGTCGGCACTGCCGCCCAGCCCTACCCCTCTACCTGCAGCAAACTTTTTCCTTCTCTGCCTTCCCTTCCCACC
TCGTCCCTCCAGCAGGTCACAGAGCGAGGGCCGCTCCCAGGTGGCCCAAGCGTGCTTGGTACCCTTCCCTGACCGGGGAGGCCTGACGGCCGGG
TCCCTGCTTCGGTGCCCGGAGCCCCGGCTGCGCCTGCCCGGGAGCCGTCAGCTTCCTGGGTAGGCAGTGCCCGAGTAACTGGGGCTTTGAGCCT
GCCCGCCTGCCTCCCTCCCTCCCTCCCTCCCGCTGCTCCTTCCCCCGCGCGCTCTCCCCCTTCTCCTCTCCCCTGACTGCTTTTCCAATGCCCT
GACATGAGGGAGGAAGATTTAACTTGGCAGCCCAAACAAATGTTCTCTCAGAGGTCCTCCAAGAAGCTGGCGGCTGGGCTGGCGGGGTGAGGGC
ACGGCTCGGCCAGCCTTTCCCTCCTCTCTGCTCTGCCACGGACATTPGCAGCCCTCTGGGTGGACAGCCCAGGGCATGGGAGAGTAGGGCCCAG
CCCCAAAGATTTAAAAGGGCAGTGGGCGGCCGCCTGTGGAAGCCCCTGGCTCCGCGCCGGGTGCAGACCCGCTCCGAGCTGCCTCCTGGTGCCA
AGGGTTTTCTGAAGGATTTTCTTGAGCAACAGTTTGTCTCCCTGCTGCAACCTGGGAACCGGGCCTGGTAGACCAGAGGGTTAACCAGTGCCGA
CTCCTCCTCCCTGGTGGGTGTGAGGACAGGCTGGTTACACAGGGCAGGGCAGGAGGCCAGAGGCTGTTCCCAGAGGTGCCTGGGTTTGCTTATT
ACTGTTTTTGAAGGTACAGATAAAGAGGCAGGCGCCGGGGCTGGAGAGCCGGGCAGCTTGGCGCTGGTGAATTCCAGCCTGCGTTCGCTGGCGC
TTGTACGGTCCCGGGAGTGTGAAGCTGCTGTTGAGCTATCGGGGAAGTTGAGATGAGAGAGCAAGCTCTTTCTTAGCCTTCTCAGCCCCGGCTG
CTTTGTGAGGGGGTCCAGAGTGGGGGAAATTCCTGGCCCCTCACTTGCTGGCTCCTGGCCCCAGTGCAGGGGCCGGGTGGGTTTGTGAATACCC
CAAGCCTTGGGCTGACCTTTCCCTCTTCAAGTCGAGGATGTTGAAACATCAGCATCAAACAAAAGACACATTGAAAGCCCTATCTGCTACAGAG
CACGTGTGAGTTGGGGGCCCAGGGAATGCATTTGGAGGGAGATGGTGTTTTCTTCTGAGCTTTATGATTTTCGGTTGGGTTTTGTTTTCCCCCA
AAGGACAGAAGCGTCTCCTTGTCCCAAATCACGAGGTTTTGAAACGCTGTTTTCACATGTAAATCTCAAAAGTTTAGACTTCTGAGAGTTGGTG
GCAGTGACTGGGGGTGGGGGCTGGGTGGGGGGAGGAGAAGGGAGCAGTAGGGAGTGGATTCAAGAGGTGGATTTGGCATTTTGGGTAGGGCTTA
CTGAGAAGAGTTGGGAATAAGAAATTGCTGGACTTATGAATAAATAGCCTTGTCTATGCAGAAAAGGGTTGCTGGCTTCTTTGTGGGGACAAAA
TGTCCCTTTTCCCTCCTAAGCAAAGGGGGGAGGAGGGTTCTTGTTCCTCTCACAGCCAGGGGCAAAATGAAGAAAGGTCCAGAGACTGCGGGGT
TTGGCGACAGAACCAGTTTCTCCCTCACGCACGCGCGCACGCACACTCACTTCCCTAGCCACAATAACAACGATGTCTCCATCCCTGGCCCCCA
TAGATATGCCTCCAACATAGGACAGGTGTCCATGCTTGACTCATTCCAGAAAATTGCAGGGGCAAGGACCCAACTGAGAAGAGCTGCAGACTTC
ACTTTTTTTCCTGTTTCTTCCTGATGGCGGGAGGGGTGAAGGGATTTAGGAAGGAAGGCACTTTGAGGCCTGCTGCATCCTGAGAGTTTTGTCA
CAAGAATACCTGCGTATAAAGCCACTGACCCTGAGGGTCCAACGAGTGGCCATGACCCTGGCCTCAGGGTGAGGCATTGTTGTAAGAAATTCAG
GCACCAACCAATTAATTTCCAGAAGCTTCTGTAGGCACTTGGTCGTGGGACCAGGAAGCAGGGCCTGCTGGTTCGTGGCTGGGTCCTCTACATT
TTCATTCATAACTTTGCTCCCCAGGGCCCTGGCCCAGCCCCTGTCACTGCTGCCTGGTCTTTATGAGGCTCCTTAGATTTTGTGTGGACAACAA
AGGCCAGGCTGCCACGATTACCCAGGACACTGCTGCAGCCTTGCCTGGCGACAGCTCTGACCTCTTAGCAGGATATAGTGCTCTCTGCTCATGC
CCTTTGGGGGAGTGCTCTTTGTCCTTAAGAGTCCCTCAGGATGGGGAGGCAGCAATCCACCTGGCTGTAAGCTCCGGGGCGTTCTGAGGTTGGG
CCCTGCCTTTAGAGAGGATGAAGGGAGGAGGCTGAGAAAAGCAGTCCTAGGCATACGCTCTGGGGAGAGGCTAGACCCCATCATGATTGAGAGG
GTCTCCCTATTACCGGAGGAGATGGGCGAGAGGACCAAGGTGTCAGGAGGCTGTTGCTTGCTATCTGTGTTCGGCCAGTAACTCTCCTGTGCTG
GACCTTAGTCTCCTGATCTGTAAAATGGGTCTGAGGGCAGAACTCCTCCGATCCCACTTGGTTGCTGGGAGAATGAGGCAGATGTGGGCATGAG
TCCCCTAGGCATGTGTCCCTTGAAGGCCTGGGAGGCTCTGGGTACAGAATGGGTAGAACACAAAGGGTTTAGATTGCAAGTGCTAGAAGCATTT
TGCTGAAGCTGCTCTCATCCAGACCTACTTCCCTGGCAGCTGATTGTCCAGTCTTGGCTTGCATGCCCCCCACAGGACAGGGTACTCATTACCT
CCTGGAAGCCTCTGCCTTCAGTGGGCAGTTGTGAGTGTTAACATGTTCTGCTACATGCCTTCTGCAAATCCACACCTCCTGACCCCACTCTGGG
CTTGAGATGCTGGGGGGCCCTCCAGACCATAGACTCGTTCTTTCCTGAATGATCCTGGGTTCAAGACCAGTGCCTAGGCAGGCCCCTAGGTAGG
GGGACACATTCAAGTGGCTGCTTCTAGCCCTTATTGGGAATGACCTGTTGTGGGGGGTGCAGAAGGGGGAAGGGTCTGCCTGGGGGTGCTCACT
GTCATCTGCCTTTGTGTGCCAGGTTTGCCTCTGGGCACTTTCCTTGGAGCGGGGTATGAGAAGGAAGGGGGTGCGTGATGAGGGCTGGGGTTGG
GTCGGGCCCGGCATGGGTGCCACAGGTGTTGACTGTGAGTGTCCACCCTGTGGATAAAGACTGTGTCCTGGGAGAGACCAGAACTGTGGAAGAC
ACTTCCTGTTCCTCAGGAGCCCACAGTCCAGTGCCGGCAGGAAGATCAGTCCAAATAAATAATTAACCGCTACCAGCTGGCTGCCTGCCACGGG
CGCGTGATGACAGTAATAACCACAGCCCCTCTCCTTTGTATGGTACTTGGTGGATTTAGAAGCTTTTTCCATCTTTGTTTATCCCCAATAACAA
CAGTGTAAGCTAGTTACTATTTGGAATTATCCCCATTTAACAGAGCAGAAAACCAAGGTTCAGAGCGGTCAAACAACTGGTGGCAAAGTTAGGA
CTGAACCTCAGGCCTTACCGATACCAACCCCTGCGCTGCCTCCTTTCCAGTCCACTGAGTGAGCGCTCCAGCCAGGCTCGGACACGGTGCAGAG
TCAGGATAGGCTCCAAAACTGGGTGCTGGGGGAATGAGGTGAAGGAGGAGCCATTCTCAGGAAGGGTCACAGATGGGCCTCACCTGTCTTCCCC
CTCCCATATGGCAGCACATGGTCCATGCCCCCAGTAGCTGTGTCCCTTTCTTTTGGCACAGTGTCTTACAGCCAGAGCTTGCTTCGAGACACAA
ATCCTGCCTTGACTGCCCAGGGCACACAGGTCTATACATGGGGCTTCATGCCCTTCTCCGGCTCTGGAGTAGGAGGCAGAGGTGTTTCCAGCCC
CCCGGGTCCCCATGAAGCCCAGTGCTCTTCCTTGGGTTCCTATGGTTTCTGTAGTCCCTCTTCGGGCTGAGTCAGTCCCCAGGCTATGTGGGGC
ACCCACTCCGTGTGAGACCTTAGGTTCTTTCTGCCCCTTGCAGCCTCCACCCTTGGGCCTGGTTTTCTGTAGCTCAGCTGAGTGCCACCCTAGG
ACAAGGCTGTAGCTAGTGGGGTTCAGGCCGGGGAGCAGCCGGGCAGGCTGAGCTGGCACCTCCCCAGGAAGCCTGGCCCAGCCTGGCACACCTG
TGCAGGTAATGACTGTGGCTTGGGAGGGCCCCAGGCCCTGCCTGTCAGGCCCAGCTGCTGGAGTTACTGCAGGATTGTGCTGGGGGAGGCTGGG
GTGTCTGAGCTAATTAGATTTCTTAGATTTCAATGCTCCCCTAATCCTGTTAGTCTGGCTGTAATTGTAACCACTTGAAGGTTTCGGCTGTGGC
AGGCTGTTGAGGACAGACGGTGGGGGGACTGTGCTCTGCCCCAGCCCCAGCTCCACCCCTAGACAGCCAGCTGAGGGCCTGGGGACTGCCTTCT
TAGGTCTTGAGGATGGGGGCTTGAGTGATGGTGGGTGTCGTGTTTTTCTGTAGAAACTGAGTATCGCAGACTGTCAGAGTGGACATAGCCCTTT
GAGATCACCCAGCTCAAACCCTTCACTGTTCAGATGGGGAAACTTAGGCCCACAGAGGGACAGAGACTGGCTCAGGACCTTGATGGCTGGTAGC
AGTGGAGCATCAAAGACTAGTGGTCTGACTTTGGGATATTTGTCCCTAGGCACTTCAGGGAAAGTCTGGAATTTCCAGGGACTAGTGAACCCTC
CAGAGACTTCTGCCCCTAGAGATAGGGCTTGGGGGGCTTCATTTGTCCCCAAGCTTGTTCCCCAGTTGGCAGTTACCTGTCTTATATTCCCCCA
GCCCTGTGTGTGTGCACACCTGCACACAGCTGCACACACATGCACACACATGCACACCTGCACACATATGCACACATGCACACCTGCACACATG
CAAACGCATGCACACCTGCACACACATGCACACACTGGCCCTGCTGGCCAGGGCTTCTGCCAGGACTCCGGCTGCCTGGCCCAGGGTCCTGAGA
CCTGAGAGCTCTCTCCGTCCTCCCCCACCACTCTTAGCCAGATACTTGCCCTCTTGGAGCCTCTGATTCAATGCAGATCAGGAAATGGCCAAGC
TGTTCTGATGGGGGTGTGTGTTAAAATACATACACTGTACTTTCCCCAGCTCTTTCTTCGAAAGTTCTGTTTTGGTGTCTGGCTTAAATGGAGA
TCTGTCCTCTCTCCCCGCACCCCATTCTCACTTCCCAATCCGCAGCGGATGACTGGCAGGCAGGATGGGGACGGTCTGCCTTGGATCTTCCCAC
TTAGGTCCTGTGCTGCCCTCCTCTTAGCTCGGGAGCTTCGGGGAAAGATCTGGAAAGCCCCTTCCACTGTGAACCTCTCTGACTCTGGGGTTTG
AATTCCTAAGCTTGTAGCATAAAATTAGTGGGAACTGATTGCGTTACCTGGTCTGGCCTTTCCTTTCCTAGCCCAGGAAACTGAACCATGGAAA
GAAACACTTGGTCTCATCCAAGGTCAGCAGCGAATGGGTGACCGATCTGGGCCTGGAACCCCATCTCCGCCAGTCCATTAGCTCATGAAACATG
TATTGAGTGTGTGCTGTTGGCCTGGCACAGCCCCCCTTAGCCACTCTTGAGCTCATTAATAAATGCTCTTCGGGTCTGATGAGAGGCCCAGCTG
AACCTTGGGGGTGGGGTTCACTTCTGAGTGCTCAGGGTGAGGACGAAGGATGGGGAGTTTTCAGTGGCATTCCCAGACTCCCAGATCCTCTGCA
GCCAGGGGAGTGTGGAACACTCCTGGTCTGGCATTAGAGCCCCTGCCCAGCAGCATGTGTCTITACAGGGTGGTTGCATTGTGAATTTGAGAGA
AGTAAAACAGGGCCGGGCACAGTGGCTCATGCCTGTAATCTCAGCACTTTGGGAGGCCTAGGCAGGTGGATCATGAGGTCAGGAGTTTGACACC
AGCCTGGTCAAAGTAGTGAAACCGCATCTCTGCTAAAGATACAAAAATTATCTCGGCATGATGGTGGTGCACGCTTGTAGTCCCAGCTACTCGA
GAGGTTGAGGCATGAGAAACACTTGAACCCATGAGGTGGAGGTTGCAGTGAGCCAAGATTGCACCACTGCACTCCAGCCTGGATGACAGAGCAG
GACTCTGTCTAAAAAAATAAATAAATAAAACAAAACAGAAACCATCTAGGGAGGGTACAAGGTTTCTCATCTGCCGGGACCTGTTTAAGCCAAG
AGGATGGTAGCTTCCCATACCTCCCCTCTCCCCTCCAGCCAGCCCTCCAGGAGCTGACCTTTGTCCTTTGCAGAGCAAATGACCCACTGACTTG
GTCCTGCCTTCACTTCCTCTGTGTATGGGGAAGCAGGGAGGTGGCCTTCTCACCCCACCCTAGGAGTCACCTGCTGAGAGTCACCTTTGTCAGC
CTAGGTGAGAGCACACTAGGAAGAGCCAGCTAGGCTCAGGACAAAGGAGGAGATGCCAGGTGCCTGGACCTTCCCCTGTCCACAGGGGTCCCAC
CCAAACTCCTACCCCTACTCCCCGGTGGACCTTGACCGCCGCTGGGAAGCACAGTGCACAGCTCTAACCACTCAGCGACCTAGCCAAGGATCAG
CAGGGCACAGAGACCTCTGGGAAGAGTCCTGAAACCCTTTCCTGGTGTGTGCAAGTGGGACGCGACCTTATAGGGTCTCTGTCCAACCTTCTTA
TTTTCCACATGGGGAAACCCAAAGCCCAGAGAGGTGGTGGGATTGGCTGGGGGTCATACAGTGGGGTGTCTCACTAAGAGTGGGGGGTTCTGTG
GTCTGGGTGGGGAGGGGTGGGGTTTTCCATCGAAAAATGGTGTTCCATCGAAAAATGCTTTGGTGTTCCTGCTGTTGTTTACATACTTAAAACA
ACTTGCCAAAGGTCTGCCATAAATATAATAGCCAGCATTTATTGGGCAACTAATGTGTTTCATGCTTTTCCCAGACTGCCTGGGTTCAGCCCTC
AGCTCTGCTCTTTATGAGCTCTGTAACTTTGGGCAAGTAATGTTTAACTTCTCCGACTTTGGTCTCCACATCTGTAAAACAGGCTTACTAGTGG
TGTAGTAATAACTAACATACTACCTCCTCCATAGGTTGAGAAGATGGAGTGCGCAAACTTCCTGGCACATAGTAAGTGCTCAAGAAAAGGTAGC
CCTAGTCATGGTCACCACCAGCATTCCCATTGCCTTACCTCTGATTTACAGATGAGACGGTGAAGTCGATTGCCCAGGTCATGTGGTGCCTGGA
GCCCAACCAAAGCGGCCTGCTGGCACCTAGGCTTCCTGATCTCCAACTCCCTGCCGTTTTCCACACTCTTGTGTAGCACAAAGGGCTGGCCCAT
CATCTCTGAGAGGCCCCCAGTCAGTCATAACCCAAGTACATTGTAATTCAACCTACTAGGGCAGCTTGTTCCCCTTGGAAAGGCGTTCGAGAGG
GAGGTGTTGCATCTGGGCTTCTGTCTGAGAAGCTTATTTTTTTCATGTGTAAACTGAGGCTCATAATAACAATACCTACTTTTTGGACTCTTCT
CGTGTGAAGGGAAGGAGGCCACAGAGGGACTGGGCTTGTCAGTTGTACCCTATTATGTGGAATCACTATGAATGGTAACTGACTGGAACTTCTG
GCCAGTCACTCACCCCTCCGAGCCTCAGTTTCCTCATCTGAAGACCATGAGCCGAAACAACATAATCTCTTCCCGAAGTGGCTTCTCAGGGCCA
CCAGTCCCCAAAAGCAAAAGAGAGGGCAGCAGAGCACCCCCACCCCCAAGAAATTGTCAAACCTATTTGCCTGGAGGAGATGGCTCAGAGTAAA
TAGTTTGCTTCACTCAGCCTTTCCCCAGACTACAGACACCACCCACCCCCGACTTTTTTTTTTTTTTTTTTTTTTTTGGAGTGCAGTGGTGCAA
TCTCAGCTCACTGCAACCTCTGCCTCCTGGGCTCAGGTGATTTTCCCACCACATCCTCCTGAGTAGCTGGGACTACAGGCATGCGCACCCATGC
CCAGCTAATTTTTGTATTTTTTTTTTTTTTTTTTTTGCATATGGTGTTTCACCATATTGCCCAGGCTGATCTTGAACTGCTGGGCTCAAGCAAT
CCACCTGCATCTGCCTCCCATAGTGCTGGGATTACAAGCATGAGCACCATGCCTAGCCCCACCCTTTTTTTAATGCAAAATAACTATGGAAAAG
ACCAATAGACCATTTTGGGAAACGCCCGCGTAAACAGTCCCTTTCAGCTCAGGCATTGCAGCTCCATCCAGCGGATGGTTTACCTACTGAAGAA
CCACCTTTCCAGGGGAGCCTTTCACAGAGGAGAGGAGGGGACAGTGTCTGCAGAGGCAGAAGTCTTCCCCTCAGCCTGCTTTCTTGGCCTTTCC
ACCTCTTTTTAACCCACCCTCCAGCCTCCCACTTTTGCCGGCCTCCTTGTCTTTTCTTTTTTTGTTACCAGACCACAAATCTGTTTGTGGTTGG
GCAGTCCCAAGAGCAAGGCCAACTGGGCAGAGGGGTGGGGCCTCTGCCATGTCCCTAGAGACAGGCCCCAGAGTTTTGCTGGCTGTCAGAGCCA
GAAGGGGTTTGTTTCACCCGTGGGGGCTGTGGGCCCCCAGAACCTGTGGTCAAGCCTTTGCAAACGTGCACTTTCCTGGAGAGGTGGTCTGAAG
CCTTGTTTCAGATCCTTAATTCAATGTGGACACCTGGAAACAGTGTCGAAACTATAGCCTTAGTCCAAGGGTCTGTAGTCCCTGGCCGTTGAAT
GGAGTGGAACTGTCATGCTGCCTTTCTCCCCCTCTTCCATTCCACCTCAGTCGTGTGACTCTTGAAGATGTGAATAGATTTATGTTCACACCTC
CTGCTTCTAATCCAGCTTGACATGTTCCTGGCAAGGAAACTGTGACCAAGGGATGTGAAGCGACTTGACCAAGGCCACAGGGCCTGGCACTGGG
GCCGGGGGGGTCCAGGGAATGATGAGTGAGTGGCAGGCAGGCATGGCCTTCTGCAGCAGCTGACTTCACTGTGAAGCCATCCCATCTTGTCCTC
TGTGAGACCTACTAGGGGTGTTGGGATTCAGTGTCCCAGAAAGAGCTGGCACTGGGGTGAAGGAAGTCCCCGCCCAGTCCCTCTTCTAGGAAGG
CTGGGCAGGACTGGTTTAGGAGTTGCCTCCTCTGGGTCAGGAGGTCAGCTTCTGCTCTGCAGGCTGCATTTTGTGGGTCCCGCCCAGGTTCATG
GTTCCTCTTCGGGGGCTGTGGGGACCACTGGCACTTATATTGAGGGGCACACGGTGTCTTCAGATTGCCCGGCTGGTGTAAGCTGGTTAGGAGT
CCTTAGCTGGGGTTGTTGTGCTGTTGGTGGGGAGACGCAGCCTCGGGCCTCCAGAGAGCCGCTGGCCGGCCACTCTGTAATTGTGCATGAGTCA
CTCTGCCTCCCTGCTCGCAGTCCTCATGTCCCCACCTTCCTGGGATCCTGAGAAGTGGGCTTTGTGATGGTTGGGAGCAAGTAAGGGGATTTCT
TGGCTCTCCGCCTGTCTGCCCTGTGTGCCCGGCATACCTGAGCTCCACAACCTCCCTGCGCCTCAGATCCTGGGAGAAGAGCAGAAAGCTCGGG
TGTGACTTTTTTTTTTTTTTTTTTTTTTTGAGACTGAGTCTTGCTTTGTCACCCAGGCTGGAGTTCAGTGGCACGATCTCGGCTCACTGCAGCC
TCTGCCTCCCGGGTTCAAGAGATTCTCTTGCCTCAGCCTCCTGAGTAGCTGGGAATATAGGTGCCCACACCACCATGCCCAGCTGATTTTTGTA
TTTTTAGTAGAGACGGGGTTTTGCCATATTGGCCAGGCTGGTCTCGAACTCCTGACCTCAGGTGATCCGCCCGCCTTGGCCTCCCGAAGTGCTG
GGATTATAGACGTGAGTCACTGCGCCGGGCCTCAGGTGTGATTTCTTGTTTGGTGTTTGGCGATGGGGCTGTGAAGCTGTGAGTTTGACGTCAG
TTTATCTCCTGCAAAAGAATGGCCTTCTGATTCCTCCCACCTGGCCTGGGTTGACATCTGCATTTCTGCTTGTGCTTCATCTGCAGGACTGACC
CCTTTAGGCAGAAGGAGTGGGTCTGCATTGGGGGTTGGAATCCTCATGGGCCTCAGAAGTCTTGGCGCCTTCTGCCTTGTGGATGGCCTCCCTC
CTAGCCAGCTCCCCAGAGGGGTCTGGGCAGCTCTGCTCCACCCGAAGGCTCTGGATGCTCCTATTTCTGGTATTCTTGGCACCTTTGATTCCCA
AGACCATTAGGGACCTTGGCGCCCATCTCATCTGATGCCTGTCATACGGATGACTTATGCCCCATCTACGCTATCCCCATAGAGCACCTTCCCA
TGTGGGCTTGCATGCCTCCACGGACAGGCTGCTCCCTGCCTCCCCAGCAGCTCCCTCTGTCTAGAAAGTTCCACATCGTATGGAGCCCAGAAGG
ATACCTCTCCCCTAGTTCTCTTCTCAGGACCTTGCAGAATGTGGTGAATTTGGTCCTCAGGAAGTCTCTTCCATTCTCTGCCTCAGAGATTGTA
TCCTCGGAGACTTCTCTCTGGGCTGAGTCTAAGGCTGGAGAAGGCAGAATGGGCTTCCTGGGGAGGTGGCCTTCTTATATCCCCTGGGCCTTAT
TATCTCTGCCTCTGGTAAGAGTCTGGGCAGCTTGGAGACCTGAGACCTGACCTGAAAATAGCCTAGCCCAGGTTGTAAGACGTGGCAGTAGGCT
GGGGAGGCCACAACAGTGGCTCACTGGGGCCCTAGGGGAAGAAAGTGGTCTGGCTTCTGCTTTTCAAGCAGGGTCAGACATTCCAGGAGGCTGA
GAAGGCCGTCTGTCTTCCTAGCCTCTTGCAGCATGCTCGCCTGCCACTGCAGCCAGAGCCCCCTCACTGGAGGTAGCCAGGGGCTGTGAATAGT
GCTTCCTCCCCTGTGGACCACCACCCAGCTCCTTACTGAGCTCCTCTCTGCCTGGCCCTGCCCCCTGCCACTTAGGCGCATCTTGCCTAAGTGT
CTACAGGCCCCTGAGACCTGGGCTCTGTGCAAGGAGCGCTGGATTTAGAGACAGAACTTCTGGCCTCCTCCTGCCAGCATCGCTGGGGGCTGTC
GCACGAGTCCAAGGGGAAGGCCAGCACGTTGCATGCTGTCCAGTATCACTGCATCGGGGAGTGTAATGTTTCATTACCATGTGCAATGATGGTA
ATGGCAGCACAGAAAGGCCCTGGAGACCATCCAGGCCAACCTTCTCATTTAGATGCTGAGTCAAACCCCAGAGGGGACGAGGTCACACGACAAG
TCCCTGGCAGAGCTGGAGCTAAAATCCAGGTGTTCCCACCATCTAGCCAGTGCCCAGCCCTGCCTGTTACTCCACAGAAACCCTTTGCTGGAGT
AAGGCACAGGAGAGGTGCCTCCATGGCTGTGGCAGTGGCACTGGCCTGGCCTGGGACTCACCTTCCTCATTTTGCAAAGCGGCTGGGCCTTTCG
GCTCTGGCTGTGCCCACCAGAAACACTCGTCCTGGGGGCAGGCACTCGGTGCCTATGGTCTTGGTAAGACAGCTGGCTCCTCCAGCCTGGGGGC
CGCTGCCCTATAGCAGGACACTTTCTCTCACTTCCTGGCTTATATGGGGCTGCCTTGCATTCCGCCTTGGGTGGCAACAGGGTCCTCAGGAGCC
CACCTGAGACCCCACGCCCAGCTTTTTGGCTCATCAGGCTCATTTGGTGGCCTAGACCATGGCTTGAATTTATGGTCTGAGATGCAGCCTGGTG
TGATCCCTTCCAGGCCCCTAAGACCCAAAGATGCCCAAACTATTGGAGCAGGAGGGACCTGGGCAGCCAGTCTACTTAATGCCCTTCTCATCCC
ATTTCCCAGATTAGAGCCCTGAGGCCAGAGAGGGAAGGAATCCAGCTGCAGAGCTGCAGGCAGGATCCCATGTCCTCCCAGCCTCACTGGCTTG
GTCTCCCCCTACTTCCACCCTTTGCGCCCGAGGCTGTCCTTGGCCTTCCCAGGCTTTGTGTGGAGTGAAGGACGATGGGGGACAATGGGCAGAG
TAGCAACTGCTACTTTTCCCTCAGGGCTCCTCTAGACCTCTGCCAGGCCCTGCGTCGCTTCCAGTTCTGGGGAGCCCATGCACTGAATGGGCTG
GGAGGGTCCTGCTGGTGGGGCCTCGTGCCAGCCTGAGAACAAGGCTTCCTGGCTGGGCTTGAGTCCCCAGCACTCTGGCCCTGGGGAGGGCAGA
GTCTGGGGTCCTTTTGTGCCTCCCCTCGCCCAGCTACCCAAGGAAGGTGGATTCATGTATTCCTTTGTCAAGTTTATCAAGCACCTTCCATGTG
CCAGGCACTCTTCCAGGCCCTGGGGATACATTGGTGAATTTGACAGGGGACCTGCTTACATTCTAGTAGGCGAGACAGTCAGCAGGTCAACAAC
TTAATGCATGACATCATGCCTGAGTTATAATCAGTGCTTTGAAGAAAAATAAACCAGGATAAGGTGATATAGGGACATGAGTGGGTGATGGGGC
TTCTTTAGATGGTGTGGTCAGGGAAGGCTTCTCTGAAGAGGCTGCATTTGAGCACATACCTCAGGGAGACAGGGAAGTGAACTCCATGAACATT
TGGGAGAAGAGCTCTCCAGGCAGTGGGAACAGCATGTGCAAAGGCCCTGTGGCAGATGCTTGTTTGGCGTGGTCTGGGAAAAGCAGGGAGGCCA
GTGTGGCTAGATGGAGTGAGGGAAGGAGAGAGTGGGGAGGAAGTCAACAGAGTGATCAGGGACCAGGTGATAAGGGACAGAGAGCCTTATCAAG
TACAGGAAGGGCTTCTGACTTCATGCTCAGTGATTTGGAAGCCACTGAAGGATTTTAAGTAAAGGACTGAGGCTATCAGATGTGTCCTCTAAGA
TCTCTGGCTGATGAGCCGAGAATAGACTGAAGGGACAAGGGTGGCCACAAGGAGACCATCTGGGCAGCATTTACAGGAACCCAGGAAAGGATGC
TGGCGGCTTGGACCAGAGTGTTCAGTGGAGGTAGTGAAACAAGGTCGGATCCTGAATGTATTTTAAAGATTGACTGAGGCTGTGGCCAGGAGCC
CACTGGGAATGCCTGTCCACCTCCGTGAAACAGGCTGAGCTAGAGAACTGGCTCAAGTTGCTGTGTCCTGGGAGTCTGAGCCCTTCTCCTTTTG
TGCTTGTCAATTCCTGTGCCTCAGCCACTCTGTCATAAGGTGGCCTTTTAACAGTTGGAAAAATAGAGGCATAGTGGGTCTTCCTGATACCCTT
GTCTACCCTTCGAATTCTGATATTCTCCATTTGGTCTCTCTCCTCCCCTCTGGCTGATTCCCTGGCATTCATCCCCTGCATGTTCATACACTCC
ACTCTATGCACCATAGTTTCCCAACATGTTAGTCATCCTCTATCCCGAGGCCTTTGCATATGCTAAGTCCTCCACCTCCACTCTTCAGTCAGGT
GAACTCCTACCCATCCTTAAGAGCCCAGCTCAAAAGCTGCCATCTTTGAACTCTCTCCCCTGACACTCTCAGGCACTGAGGATTTCTTCTCCAC
TGAAAATTCTACTCCTCTCCCTCCCACACTCTATTGCAAGGGATCAACTGCGTGGGGCTGGGTCTGTGCCTTCCTTCACACCTCCTCTTTCTGT
TCTGTGTCCTCACAGCCTGGCCCAGGGCCAGCACAGAGGAGGGAACAAAAACATTTCAGTGTATTCCTGGAGCAGTAGTTGGTTACTATGGAAA
CAAGGAAGTGTTGCAACTTCCTGAACTTGAGGTTGGGGTCAAAGAGGGCAGCTCTATCTTGGCCACACGGGATCAACCCAAGATGGCCATGCTG
AGGTTCTTATTAATTCAAAGTCACTTCGGACAGGCTCTTTCCCATCCCTGAGCCTCAGCTTACCCCATCAATAAGATGCAGGTGTTGGGACTGC
TTGACTGTCTGCTCTAACGTTCTGTATCCTTGTGCCTCCAATCCTGCCGTATGAACACAGGCAGGTCCTTTGCCCTCTTTGAGCCTCAGTTTCC
CTATTTTGTTGACCTCTCTCCAAGAACATAGCTGCCTGAGTCAAGCAACTCCAGAATCCTAGGGTCAGCGGCTGGGCTGGCCGTTGATGATTGA
ACTGGGATTCTTGTGACTTGCAGAGGCACAAAGAGGGAAGTCCTGCTTTCCAGGGTGGTGGGGTGGGGGTGGGGGAATGAGGCAGCAGGAGGGT
GGGTTGGTAGGGATAATCCAGGCCGGCTCACTCTGGCCTGAACCTGCACAGGCCCCTGGGTCTCCTTTCTTCACCTTGAAGGTGGCAGGAGCTA
GTGCTTGCCAGGGGCCCTTGGCTCTGCCGGACGCTGCCCGCTCCGCATTCTCGTGCCGTGCCATGTAGATCCCGGCCTGTGCTCTGAGGGCCAC
AGGTCCTGTGGTGGCCCCAGAGGCTGCCTGCTGCCTGTGGTGGCAGGGCTGAGTGGGTGGGCACCATGTTCTTACTGTGTCTCTTCTCTCTCCA
CTTCATTGCTCAGAAGCCAAGAGTTAGCCAACCACAAAACCACAGCCTGGCTGCTGGCACCCTCCTCTGCAACCCTGCAGCACCTGGGCAGGCT
GGTTCTGGGGGCTCAGCACCAGTCACCAGGGCTCGGATGTAGCCTCCTCAAGCTGGGGGTGGGGGTCAAGGGGCCAACATGCCCAGCGTGACGC
CAGGCCCGCAGTTGAGTTCCCACCTGCCATTTGCACATGCCATTGGGCAGAGTGGGCAGAGGCCTCAGCCCGCATAACGCCCCACCCTGACTCC
TGCCTGGCACTCAGGTGCCGTCTGGCCTGCACCAGACCCAGAAAGAGTGACCTGGGATGAGAGGCCTTCCATGGCTCTCTGGGCCTGCCTCCTC
ATCTGAAAAATGGGCAGAGGGTGCCCGCCTCCCATGGCCACCAGGAGGGGAGACAGAGGAAGGCAGCTTGTGATCTCCAGAGCCCAATAAACAC
ACTTGTTAGGATTACCAGCAGCAGGCAACAGGCTTCAGCCCAAGAGATGCCCCTGAGAACCTCTTTTTCAGGAAGCAAAAGCCCAAACCTTTTC
AGAATGAGACTCTTATTCTGGGAATGAGAAGTGCCTCTCCCTCTGTTTCTGAGCTGTGCCCAGTGGCCAGGGACCTGAGTGGCCATCACAACGT
GCCTTTCCCGCCTACCACTGCTCCCTGGCTTTCTAGGGTTGGAGTTGGATGAAACCCCTGGGGCAGCACCGGGCACTGGAAGGGCCCTGGTAAG
GGAGAGGATACCCACTGCCCATCTCTGCCACTCATCAGCTTCCTGACCCTGACGAGTCCTTGTCTCTCTGCCCTCTGTGCATTGAGGCCCCTTC
TAGCTGGGACTCTCCTTTGGAGTCTACTGGAGCATTTTTACCTCCAGGATAGGTGAGGCTCCCTACTCCAGACCCCCAGGATGTAATGTGGGTC
TCACCTCAACTCACAAAATTCTCACTTCTGTTAAGAAAGGGCCTTGCCATGGTTGACGGGGATTTGCACTTCACCGTGTGGACAGTGACAGAAT
GCGTCTCCCCGCCCCTCCTTACCTGGCAAACTTTGTCATTACCTCCTCCCAGGGACCTCCTACGCATTCTCCTCTGGACTCCCTCAGCCCTCAG
CCTTCTCCTCCTCCCGGTACCAGCTAGGTCAGACAAGACCTGGAGCAGAGAAGGTGCCCAGTAAAGCCTGATGAGTTCTTGAATGATCAAGTGA
AAGCCCTCAGGTATCCAGATAGTTCAGTTGGCCAGAAAGCCCATTTTTCTCAGATTTTACTCAGCTATGTTTGCTTGTTCAGCATATAGAATCC
TTTTTATTTTTATTACTTCATTTTATTTTGTAGAAACTGGGTCTCAGTCTGTCACCCAGGCTGGAGTACAGTGGTGTGATCATGGCTTACTGCA
GTCTCGAACTCCTGGACTCAAGCGATCCTCCCACCTCTGCCTCCTGCTGGGACTACCGGCACGAGCCATCACCCCGGCTAATTGAAAAAAATTT
TTTTTGTAAAGACGAGGTCTTGCTTTGTTACCTAGGCTGGTACGATCTTTTGTTATTTATTTTTTATTTTTTAATTTTTATTTTTTTTAAAGAT
AATCTTTTTGGAAGCTCTGTGTCCAGGACTGATTCTCTATACCTTTGGCCAATCTCTTTCTTTTAAAACCAGGGTTTCCTGAATTGCATTTTTG
CATTTCTTTTATTTACCTGGTCTGTGCAACTATTTTTTTCTCTTTTGGACTCCATATAGCAAATAATTCTGCCTCTCCTTTTATTTGAATTTCT
CATGCCAAAAAGGCAGGGAGGGTTAGTAAACACTGCTTTGGTTGTTCTTTGTTTTGTTAAGTGTTTTTAACTTCTTAGTGTGTTAGATTAGATT
TTATCTGCCTTTCCTACCTGCCTTAACCCCAGGCCTCTAACCCTCCACCTTCCCATTGCTTGTTCACTGGCATTGTGTCTCCTAGGACAGCTGT
GTGTGGTAGCGCATGTTGCGCACTGCACAAGTTGGGGGTGGTACTCACATTTTAGTCCCCTCACATTATACATGTATTAGATGATTTTTTGGCA
GGTGATGGTAAAGTGTTGGAGGAAGGGGAGCCTGGTTCTAATTCTCATAAAGCCACCTTGGATTAGGAGTAGGCTGACATTGTCCCATGGTCCT
GGCTGAAATTGGGAAGTCATTTTGACTCCTCCCCAGGGCTGTACCCCGTGACATTGGGTGTCCATGTCCCCATTCCCCTGCCCAGAGCATGGCT
TATGGGAGTATGTAGTCAGGGAACTGATGTCTTTGGACTCCACCTTCTGGATGTTTCTCCAGCCACCTCTCCTGTCTACATCCCCAGGCAGCAA
CCTTCTCCCTCAGGTGTCTACCTCCAGCTTCCCCTTTTCCATCGCCCCTTCACTTGGCTACCAGTGTGAACCTGAATAAAAGGCACCCCTGATC
TATCACCTCTTCCACTCTACTTAAAACTCCTTCGTGCCTCTCTACGATCTACAATCTGAAATTCAGTGCCCTTCATTCTCTGGCCCCAGATGAA
CTTTCACTTGTGTGTGCTTTGCACGTTGTGTTCCTCTCTGCTCTGTCTGCCTGGAGAACTCCTAGGCATCCTGCAAACTCTGCCCAAGCCTCAG
CTTGCCCTCATGCACCAGCTAGGTCAAGCGGATTGGTCCTCCCCTTGCCCCGGTTCCCCAGGCACTGTTTCCTGACCAGTGTTGCAGCCCTTGC
TTGGCAGGATCGCAGCTACCTTTAGAATACCACAGCCACCACCAGGCTCTGGCTCCCTGTCACCCAGCAGCACCAGCTTGGATCTAGCAAACAT
TCCAGTGCGCTCCCTGCAAGTTGAATAACACATGGTGGCTACCCTTTCCCAGAGCTGGTCGGCTTTGTGATGTGGACCATGTGCATGGTCTGAC
CCCTGGCCCCTTCTTTTGCCCTTTCCCCTGCCAGCCAAAGCCAGAGAGATGGCTGGGCCATGCCAGGCTCCCAGGAGCGCTTTGTGGGTCAGGT
TGTTTTGCCCAAAGCTACTGGGTCTGAGGATACCTGTGAGCTTGCTGTGCCTGCATTGCCCCTGCCCAGGCTTGGCTTCCTGGGAGAATTGGCT
CGGCAACAGCAGCCTGGGCCAGGCCACAAGGCTTCTTGGGCAAAACTCCGTGGGCAGGGCCAGGGGCTGGGGCCTCATATGCACTGCCTGTGCA
CCAGGCGAGTCTGTGGGCAAATGAGCTCCAGCTTGCCATGGTGCTGGGGGAGGGCCACCCTTCTGCTAGGTAAACATGGGTGTTCAGGCCTGCC
TAGGCACGGCCCTGGGTTCTTTCGGTTAAAGGTGCAGGACAGCATTTTGTCGAGCCGGGAGTTGCCAGTGAGAGGGTGACAGAGAGGGACACAT
GGGAACCTTCACCAACCTTCCCTGGGACTCGGCCCCTACAGGCTTTAAGCAGAGGCTGCAAGGAGGCTTAGGGGTCCCTGGGCCAGGACTACTG
TTCCGACTCTGAGATCTTTTATCTGCTGCAATGCTGGCTGCTTTCTGGGACTTGGGGAAGGTGGGTGGGCTGTGAGCAGGTGCGTTTCCTAAGC
CAGGTCTGAGACCAGAGCTTCCATTTAGCGGTCCCCACTGCAAATGCATTTCAGTCCTTGCTCCCCCAGGCAAAGCTCACATCCTTGGGAACAT
CCTTCCTGCACCAGGCATGGTACTTTGGGGTCTGGAGACTGTCGCAGCACCCCTCTGAAGTGGGTGGTATTATCCCATTTTGCAGAGGAGGAAG
CTGGAGTTCAGAGTGCTGAACTTGTTCAGCCTCCACAGCTGCTCAGGGAATCAGGTCTTCTACTCCCTACGTGAGGCCACAGCTTGATTTTTCT
TTTCTGGTCCGTGGCTGTTTGTACAAACGAGTATCCTGTGGACATGGTTGTCTGCCCCTGCCCCTCCGACAGGACAGCGCTGAGCAGGTACCTG
GGCTGGTGCCACACTTGGGGAGGTGGACACGTAGCCTCAGAGCCCGAAGCAACTGGTGGGGTTCATTCCCATGGAGGAGAGGTTCTCCGTGGTC
CACGGCCGGAGCCTCCGCATGTCAGATCCTTCTCCCCACCCAGATGGAGCCAAGTGGGCCCCACATCAGTGAAGGAAAAAGCCCTTGGCTCTTG
TGCAAACACAAATTGTGGAAGAGTGGCATCAACAACACGAGGTGCTCCCCATCCATCCGGCTCACTTTCCTTCTATGAGATGAACTCAGCTCAG
GGTGTTTTGTGCCTTCTCCGGTTTGCTCTAGTTTGGTTTTGGTCTGGTACAATTGAAGTCTTTGCCTCCTTCCGACAGAAGCACTGGACCCCTC
TCCCTGGGCCCTGAAAACCCGGAGGGGTACTGGGACTGCTGTGTTGGATCAGAGGAGTCAGGTAGAAATTGGGGGTTCTGGGTGCAGGGAGCAC
TTGAGGTGCCTGCTGCATGCTCCCTGAAGACAAAATCCCAGGGAGGATGTGTCAGTTCCGAGGGCCACTGAGATCACCACCTCCGTGAGCATGA
GCAAGACCCTATTGGCCGCACCTGGGACAGCCTTCCTCGGCAGGGCCAGTCCTACCACCTCCTTTGTCCCTCTCCCACTCCCCAGACCCCCACC
CCCTACCCCTCAGGCCACCTGCTCCCTTTTTACCCAGGCACATGTCCATGTGTGCCCTTACAAGCTCCTGGCTTTCGGAAGGGAGGGGACAGGT
TGTAGAGAGACGGGATGCGCCGGAGTGTAATTTTATTTCTGTGGCTCTCCTTTCAAGCTGTCACGGTAAATCTATTTTACTTGTCACTTTGTAA
ATCATTATGCAGGATTAAGCCTTTTACCAGCTTTTAAGGCTTCTGCAACCAGGGACAACCCCACCTCTCCCTCATCCTGCCTTCTTTCCCTCAC
CTCCCTTCTGAATGACCCTACTGCTGTGTCTTAAGGCTCAGTTAGAATGTAAGTGGGGACCCAGGAGACAGCTTGCTGTCATTGAGTTTAGCTG
CCCCATTTTACAGGTGGGAAAACAATCCCTGGAGAGGGAGGGACTCCCAGGTGCAGTGCCTGGGGTGCTTAGTGTTTTGTGGTGCCCCTGAGCA
GTGGGGAGGGAAGTTGCTGAAAGCAACCGAGCCTGGGCATGGGACTTGGAGGGTTTTTTTTCTCATCCCCCATGGGGACCTGGAGGCCCCCTGT
GGCTGTGGCAAGATTGCTCAAGACCCACAAGGAGACCCCGTTTGGAAGAGGAGACATACTTGGAGGGAGAGAAGCACCTTCTGCCCCCTCTCAG
GGTGTGGGGCTGGTGGGGTAGGGGACTGGTGACAGGAAGCTGGAGGTGGGAATGTGGTCACAGTCACCCTACTGGCATGTGGGATCCCGTTTAT
TTATTTCTCCAGAAATCTGTGTCCTTCTCCAGAGAAGGCTTTGAGGTGCAAGAACAATGAAGCCATGTGCTGAGATGTTGAGAGTTGCAGATAC
TGGCTGGGACTACTAGGCCAAGAGCCAGCAGTGTGCAGCGGTGCGTTTCCTTTCACGGGTCCATCTGGAGCAGCCTGCTCACAAAACCAGAGTG
AAAAGATGCTCACCCACAAGGCTCATGTGCATTGTGGGAGCTGAGGTTTGGGGCGTGCCCACTTTGAGAAGCCAGGGATGTATGTGCCCAGGGC
CAGACTGCCCAGCTGGGCCATGCCGGGTTTGACAGCCTTAGGTCTTGCTGCCTTTTCCATCGGGATACACTGGCCTCATCGCCAGGTCAAGGGC
TTCCTCCCTATTGCTGGGGTTCCAGGGCTGTCCTCTGCCCGTCTGTGCCAATAGAGCGCCCCGCCTTGGGGGGCCCACACTCACCTGWCCTGCT
TCCACCACTCCCCACCTAGACCTCTGCTGTCTTGAGCCCAGATGAATTAATCTCTTGCAGCAATTTCAGTCTACCAAGAGACCTTCTCCAGCAT
CTTCTAGGGGAGGGTCCTGTGCTTTGGGGAAGCTGGACTGGGTGACATTCATTTCCACAGCCCTCTTTGCTTTCCCTGTTCAAGCCACCATGAT
GTGCAATGGCAGCTGCCTTTTTTTTTTAACTTAAAATTTTAAAATTTTGTGAGTACATAAGTAGCTATATGTATTTATGTGGGCAACTGCCTAT
TTATTGGCCTATTTCTAGACTCGAGCTGTTCTGCAAAGCCAGAGCCTTGTCTCTTATCCCCTAGCACGGGGGCCATAACGGCACAGGCTAAGGC
AATATTTCTCCATTAGAAGGATGGCCAAACAAGAGTCCTCAGACCCTGACCTCAAGGAGTTTCACAGTCTGCATTGGAAACAACCTTGGCTACT
CAGCAGAATAGCTGCAATGAGTACTGAAAGCTTCAGAGAAATCGGCTAATAAGTTCCAAGGAGTGAGATGAGAATGAGCTAGAGTGGTCGGTGG
GGAGGGGCCTCCTGGAGGAGGGGGCATTTAAGGGCTGCACTGAGGATGGAGAGGCTTTAGCTAGAACAAGGCAAGGAGCGGGAGGAAAGGTACT
TCTGGGCGGTGGAACCGGCATGACCAAAGGAAGGGAGGTGTGCCTGTGGGGGTGCTGGGGCAGCTCAGCCAGCACCCATGGACTTTAGGGGAAG
AACAGAGGCCAAGTTTCTAGGGATGGCTGGGACCCCAGAAGGAAGCCAGGGTTAGGAGGGAACACTTATTCTAGAAGTGGGGAGGGAGAGGTGT
GTGACAGACTGGGTGAGGACAGAGCAGTGTTTTAGGAGATTTAGTGACAGGGAGCCACCTGGGTTGGAGGAGACCTGAGCCTATTAATTAGGCA
GGTGAGTGGAGTCCCTGAGGTGTACTTCAGGACCAACCAGGCTCCCTGTACTGGGTGGGATGGGGCAGGGCTTAGGGGCTGGTGCTGTTCCCAC
CAGCCCCGCTCCATGCTGAGTGGGAGAGGAGGTGGCAGCAGCAGCTGGGGCTTCTGAAGATCTGGGTGCAGGCTTGGTCTGGCTACTCCTGTGC
CTCTGTCCTCAACCTGCAAAATGGCCAGATTCTGCAACTCCACCCACCCTGTCTCCCTCCTGCTGTGAGGAGTGCTGGACACACAGCGAGGGGA
AGTTCTTTGTGAACTGACATTGCCTGTTCTCATCCCCTTCTGGAAAACTGGTGGCTGCCATGGCATGGCCATTTCATGGCTGCTGGCTTCCCTC
TGTTCTCCAGATTGGGGGTGACCCTAGGACCTGGGTTTCATTACCAACAAGCCATGTGATCTTGGGTCAGTCACCTCCCTCTTCCAGGCCTTGG
CGTTCTCCTCCATAAATGGATCCGTGCTGATAACCTAAGCCTCGCTGCACGCAGTGTGGCTGTTGTTGTGCTAGGAGACCCAGGCTTGGGCTGC
GGCTAGGGGTGTCCCCCTGTGGGCAGAGCCCCATACCTGTTGTCACAGTAGCATCGCAACACCTCCTTATAAGCCTTGGAACCTCGGCCAAGAG
GTTTCTGTGTACGCTTCTTGGTCTCTGGTGACTTCTACGCCCATTTGACATTTCTTTGCACTTCCTGCCCACTCCTGGAGGCCCCTGATTAGTC
TACTTGGTTACAAACCCATATCAGATCCCTGATATCTCCCAGCAAGCCTTGCACAAGGCATGTGCCTTTAAGTGATGCTCGTGGCTCTTTGAAA
TCTGGACACCAGCTGGCAGAGGGGTGGTCAGGTAGCTCGCTTTTCTCTAAGATGGGTGTATTTTGTTTTTTTTCTTTTGGAACAGATTTTGATT
TTCTGATAATAGAGCCCTTGAGATAGTGAGGAGACAGTACCGGAAGCAGCACCCCCAAAGCCGTTTGAATACAATGCAAGCAGTTGAGGGGCTG
AGCTACCAGCCAGTAGGAGAGAGGGTCCTGGGGGTTCAAAGAGCAGTGGGGGAAACTGAGGTACAGAGAGTACCCAGCAAGAGAATGAATGAGT
GGCAGGTCCAAGCTTCAGATAATTATTATTCTTTCTACCACCCCCCCCACCCCCAACACACACACACACACACACACACACACACACACACATA
TTCAGGGGATGCTTTAGGTTCTCTGCACTGTGCCATGCAGTTTATGTGCATTATCTCATTTACTGTTCTGAAGAACCCTACGCGGGAAGGACTG
CTGTACCCCAGTTTTATTTTATTTATTTATTTATTTATTTATTTATTTATTTATTTATTTATTTATTTATTTTGAGACAGAGTCTCACTCTGTT
GCCCAGGCTGGAGTGCAGTAGCGTGATCTCAGCTCACTCCAATCCTGCCTCCTCGGTTCAAGCGATTCTCCGGCCTCAGCCTCCCGAGTAACTG
GGATTACAGGCGCCTGCCACCATGCCTGGCTAATTTTTGTATTTTTAGTAGAGATGGGGTTTCACCATGTTGGCCAGGCTGGTCTTGAACTCCT
GACCTCAAGTGATCCACCCACTTTGGCCTCCCAAAGTGCTAGGATGAGAGGTGTGAGCCACCACGCCTGGCCTGTATCCCACTTTTACACACAT
GCGTACGTGATGTGCTCAGAGTGATTAGGGCCACAGAGCCAGAAGGCGTTTTAGGCAGGATTTGAATGCAGGCCTGTGAGTTTAGATCCTGGCT
CCCACTACCCTATGCACACCCTCCCATGCCCTTGCGTGCCTCTTCAGGGCCTCTCTCTGGGCCATCCCTGCCGCAAGCACTCTGCAGACAGACA
GTCTTCCTCTTTCTAGGAGGTGTGGGTGCAGAGGACTTTGTGTATTGGGGAGATAAAAGAGCCCGTTTGGGATGATTTTTATCAGCTCGAGCAA
GTCGGTTTGACTTACAGGAAGAAGCCAATTTGCATTTTGGGAAAGAGCTGAGTGTAAAGGCCGCAGTGAGCACTGGGTGGATTTATAAATAAAC
AGTCGACCGAGGGCATTGTATGTCCCCAGCTGTACAATGCTGGGTTCATATTGACACCACAGACCTGTTCCACACGTCACAGCTGCCCTTTGGA
CAGAAAGGGCCTAATTGAGTAAGAGAGCGCTGCTGCTGGAAATGCCAAGGAACCCTTCCTCCGACCTGGCTTCCTCGGCACAAGCGAGCCGGGT
GAGTCCGGCTGCAGCCGGGCCTGTTTACCGAGGCTGCTGGCATTGCATGCCAGGTGCAGAGCCCACGGGCGACTCAGAAGGCCACGAACGCTGG
GGCCAAGGCGGCCTCTGCTCTCCCTGCAACCCGGAGGGAGCTGGAGCCAGAGCCAGATCTCAGGAAACTGGGGTCATTGCATAGAGGCTGCCAG
ACAGTCTGCAGAGCTCAGCGGCCTGGGTTCAAACCTTCTCGCACACTGCCACTGTCGGTTACTTTGGCTTTGTAGAGCGAGATTCCTTGGCCAT
GAAATGGGTACTGCTTACTTCCCAGGTTATTTTGAGAATGAAGTGAGATGAAGTCAACAGTAGATGTATCTCTCCGTTGTCCCTGCCCTGCTGT
GGGATGACAGAGTGATTTTGGACAAGACCAAGGCCTCGCTGGGCATCACTGTCTTCTTCAGAAAGCAATGGGGCTAGGCCAGGTGCACAGTGGC
TCACGCCTCTAATCCCTGCACTTTGGGACGCCAAGGTGAGCQGATCGCTTGAGCTCAGGAGTTCGAGACTAGCCTGGGCAACACAGTGAAACCC
TGTCTTTACCAGAAATACAAAAACTAGCTGAGCATGGTGQTCTGCACCTTTAGTCCCAGCTACTGGGGACGCTGAGATGAGAAGATTGCTTGAG
CCCAGGAGGTCGAGGCTGTAGTGAGCCACAGTTGCGCCACTGCAGTCCAGCCTGTGTGACAGAGCAAGACCCTGCCCCTCCTGGAAAAAAAAAA
AAAAAGAGAGAGAGAGAAAGCAAGCAAGCAGTGGCGCTGGATGCTGATCAGAGGCCTGGGGCCCTTGGACCAGAGAGGGCTGTCCCTGCCCAGC
TGGCTACAGGAGCTCTGACTCTCAGACCAGCCTAGGTTCATATCCTGTGTCTTCCACTTGTGGGACCTTGGGCATTTGACTTCCTCTCAGGACC
TCAGTTTTCATGGGTAGAAAGTGCGAACGATTAAGGTTCTTGGAAAGACAAAATATGCTCATGTACACTTAGTCCTCAGCATAGGGCTGGCCAC
ATGGTGAATGCTCATAAATCAGTCATGTCTATAACATACATTTCAAGGGAAAAACATCTGTTTTGAGGGCAGAACGCTATCAAGTCAAATATTT
TCCTGATTTAATTTCAATAGTACTCTTATAAAAAACAATAACCATGATACCGATAGTTAGAAGACCGCCCCTTCCAGGCCCACTTCTTTACCCC
TTTGCTTTTTGGCAATAACCCTTGTGCAGAGCCAACCCCATTCTCCTGACCTAACCAGTTCTCACCTGGCCATGCGGCCTTICCGGGTGTCAGG
CAGGTCTCTCCGGTTAGGTAAGGGAGGCCTCATCCTTCTTACCTGACTGCTCTGAGTGCAGATCTATGCTGCTTCCCATCTTCTCCAGGCGCCT
GATCTATTACCTTGGGCTTGGAACGCACCTAACAAAGGCTACCATGAGGAGGCCCCTCGGTACCTGTTCATCTGGACACATCGTGCAGACCCCC
GCACCCCGTGTGGCTCCCGGTTAATCTTCAGGGTGCAATCAATTTCTTCCTCAGAGGCCAGACTTCATTGTTCTAAGCCTTTCTCAGATCCACA
TCTTGGGGCTGCTGGGGTCCCTGCATCTGTGTCCTGGTGGTCAGTTTGAAGCACCAGCAGACAGCAGCGCTAAAGGCACCTTGTGTGTACTACG
TGTGGCCATCTAGGGATGTCTCCTCTGTTGCTGAACCTGCAGATCCCCGTGAGGCAGGTGGGGCCACAGGCCTCTCCCCTCTGTTTTATAGGGG
AGGTAATTGAGCAGAACTTAGGACAGTTACATGCTTTGCTTCAGTTGCGGGGCTGGGATTTGGACCTGGCTCCTGGCCTGACACAGGTCCTCAG
CTTCCACCAGCCTTGCTGCCTCTGAAGGTTGCTGGAGAAAGGAAGCTGAATGTAGAGGGTGGGTCTGGAGGAGAGGGGGAGGAGATGAGCCCAG
AAGGACAAATGTTTGCACAAGGAGTTAGAAATGAGATGTCTAGGCTGGGCACGGTGGCTCACGCCTGTAATACCAGCACTTTGGGAGGCCGAGA
CAGGCGGATCACAAGGTCAGGAGATCGAGACCATCCTGGCTAACACGGTGAAACCCTGTCTCTACTAAAAAATACAAAAAAATTAGCCAGGTGT
GGTGGTGGGCACCTGTGGTCCCAGCTACTAGGGAGGCTGAGGCAGGAGAATGGCGTGAACCCGGGACCCGGAGCTTGCAGTGAGCCGAGATTGC
GCCACTGCACTCCAGCCTGGGCGACAGAGTGAGACTCCGTCTCAAAAGAAAAGAAAAGAAAAGAAAAGAAATGAGACGGCTTCAAAGAACCTCC
AGCACGTGGAGGTGAGGGGTGGGAAGGGCTCTGAAACCATTGGCCGTTTTCTTGAGTCCTTCTTGACCCGAAACGCTGCCTTGAGGGGTTGATA
GAGACTGGCTCCTTCAGTCAATGCCTGCTTTCAGACCTGGATGTGGGGGCCTGCTGGGTGCCAGCCCCCTGCTAGGCTCTGGGAATAGGACAAT
GGCCAGAACAGATAAGATCCCTGTCTGCTGGGAGCCAAAGAGAGAAACCAGGTAAACAAGACAATCACAATCAAGGACACCTTTATAAATTATG
ATAAGCACCAAGAAAGAAACAAACAGGGTGCTGAATAGAAAACCACCCAGAGGPGAGTGTGGGGGCCTTGAGACAGGAAGGCCTTTCTGAGGAG
GGCCGTGAGATGATGGGGTGGGAAAAGAGAGCACACAGGCAGAGGAAACAGCGTGTGCAAAGGCTAGAGACAGAGAGGGGTCCCATGTGGCTCG
GGGATGACCGGCAGGGGCAGAGCAGCTGAAACACGGTGGGCACCATGCATATCTTCCAGCTCCACTTGCCCCCGGGTCGCCAGCAGTGCTGCCA
GCCTGCTGTGGGCATGGGCTCAGGTGTGGACATGCCTGTATCTGCTGGGGGGTCCCACATGGTGCTGGCTCCCTGCCTCAGCCAAGAGGCACAT
GACACATGACATCACTGGCTATTTCTGCCTCCCACCGGCCACCTCCATCTTTGCAGCATGAAGGGAGGCCTGGCCTGAGCCCTTAACTGGTCCC
CTGGCTTGGCTGTCTGCCACTGAATATCACCTGCTATTTCAGTGACCATCCTGCTGTCATAGAGGGCAGAGGAGGCTGAGGGGTGCAAGGGCTG
TCACCAGGGGTCCCCGGACAAGCCCAGGTTCTTCCGCCTAGGGTCAGCCCTGGCCCTGCAACCTTTGTTTATTTACTTTGGGCCTTGCTCAAAA
CTAGTAGCAAGCTGCCTGGAATGCAATGTGACGAACTCGAGATAATAATAAAATCAATTGGACTGTTTCCCCAGTAATTCTAGAGACTGATGCA
TCATTAATGTTGGTAATACAGTGCCTGTCAGATCACAAACTTCTCAATAAATGTGCAGCTGGCAAGCTCTGATAGAAATTGGAGGCAAGCAGAG
AATGAGATTTGGGGCCTTTGATATATGGGGTGTCAGTGGCGCAGTGAAGTTTTTATCAATTTCTGTCACTTTGGGCTTTTGTGCTCACAGCATA
TGTGGTCTTCATGGCACCTCTCTGTGGGCTGCTTGGATGCTAACTGCACCCCCCCACCCAACAGTGTGAGGAAGAGGAGTTCCTGGAGAGGCCT
CAGCAGCACTGCTGCCCTTCTGTGCAGCTTCATTACCAGGGGCTTCTCAGGCAGACGTAGACGGGCAAAGCCTTTGGACTTGACTCTGTGGATC
ACTCAGGCACTCTCCAAGCATTGATTGAGTCCCTGTTGGATTCAAGATGCCACTGGGCCCTCGGAGGCCTTTGGAGGCTCTGGACGACTTGATG
GAAGCCTGGGGCTTGGGGTTGTGGGGAATGGTCAAATGATCATTCCTACCTAAGGGTCAGGAGAAAATGGAAGCAGTGTGTGAGCTAGCCTGGA
AGTGTCTTGCCAGGTAGACTAGGAGAAGAGACTATTAGACTGGGGACCTCTGTGGGCCCAGGCCCTGAAGCATGATGAATCCTGGAGTTTATGC
GGGAGTGAGGACTTTGGCCAGGCCAGGCCCCAGCGGCATTGGAGAGAAGGCAGAAGCTGCCTGGAGGCAGTGTAGTGTGGTGGTTAGCACAAGC
ACTCTGGAGCTAGACAGGCACGCTCCCAATCCTAGCTCCCACACTTAGCAGCTGGGCAACCTCAGGGAAGTGACTCGACCCCTCTGTGCCTCAG
TTTCCTCACCTGTAAAATGGAGGTGGTGATAGTTGACCACCTATCTCGAAGGACTCTAGCGAGCATAAATGAATTAATACTTGTATGAGTGCCT
GGTCCATTGCAAGCACCTGATAAGTAATTGGCATTACTTTTGCATAATGAGCTCAGTGAACCCACCAGTCACCTGAAAGCCATACAGTGAGATT
CAGAGAGTTGAAGTGGGGCATCCGCACTCATACAGCCGCCCAGAAGCAGAACTGAGATTTGAGTGCAGGTGATCTCAAAACCTGCGTCTCTAAG
CTGCTGCTGAGGCTGCGGTTGAGAAGCGTGGGCCTTGGGTGCCCAGGTGGAGGAGGTGGCATGCGGCTGGGGGCCCAGCACCCTACCGAGGACA
GGCCTGTGAGGTCACCGTCATTAGCAAGGAGATTGGGTGGGCAGTGAGTCAGCTCAGGCTTCATTTGTCAGTAGCCCTCTGGAGCTGGAGAACT
TCTGAGACTGGGGACTTGACTGTGGTGCAGACGTCTGCATTGGCCCCTGGAGGCTGGAAGTGGCTTTGAGTAGCAGAGGAGCCAAATGGGAACC
ATGTTGGCGGCAGATTCCAGAAGTGAGTCAGAATTTCAATGTTCCCAGCTCATCTGCTTGAGAGCAGGTCCTGAGACCCAGCAAGGCCTGCACC
CCCTCCTCGACACCCACCCACCCTAGAGCACCACAGTGTCTGCAAACCAGACCGGAGGGTCCCTAGGCPGTGGGGCTCACTTGGGTGTGCGCAC
ATTCTGATGCAGCTATCACTGTGGTGCACCTGCTGGGGACCCACCGTGAGGCCCCGGGAGTCGGTGTGCTGGTGCTGGATACCAGCGCTCCTTG
AGAGTGCCCTGCGCACAGCGACCCCATGCTCCCATGAGCAGGGCTGCTCCTGGCCCTTCCCCAGGCTGCCTTTGAAAAGGTCCTTCCCCCTCCC
CATGGCTCCACACAACACCAGGATGTACTCCTGGCTGGTTCTGCATCTGTGTTTGCCTTTAAAAAAGGTGGGTTGCAAGGCCCCCCAGGGCCTC
AGGAAAGAAGAAGCAATCTTGTTAGACTGGGGTGTGGAAGACAGAGGGGCCTGTGCTGCACTGGGAACAGGGAGCGGTGGCCTCTGGGCCCAGG
TCTGCCCCCATCCCCTCCCCGGCTCCCTTCAAACCTCCCTGAGCCTCCATGCCCTGCTTGGGACGTCCCAGTGTGGAGCCCAAGTCCCTGCACT
TGTTGGGTGGTGGAGGATAGCTGGTCCTGCATTTCCCAGGTGATGTGCCTGGGAAGCATCTCTCCATAAACCCCCTGTGACTTGGGCTTGGAGG
AAGTGTCCCTTCCTGCCAGAGGAGCGGGGGCTGGGTGAAGCATTGAGAGGAGCTACAGGTGCATTTGAAAAAGCAGGTAGGTCTTCTTTTTACC
ACCTGGAGGTCCTGGTTATGGGGGCAGGTAGGAGCACGGTGCTGACATGGTCCTAACCGGTGTGGTTCCCATCACTACTGCTTGTATGACCCGA
ACAAGTCTCCAGCCACTCTGAATGTCACTTATCACCACAGTGCAGGTCCTCATGCCCACCTGACCTGGGGTGTAATGGACCACACACCCTGATG
CTCAGAGCACATCTCTTGTTGAATGCTAGGCCCTGGATGACTGGTCCTGGCCCAACCCCAGGAGTTTAGAGCCTCACTAGGGAACAAGACTGGG
CATGTACTACCTGTAGCATGAGGGGCTGGTGGCTGTGAACAAGAGCCCTGCTGAGGGGAGCCCCAGGGGCACAGGTTCAGGTCAGCCTACTAGC
GGAGGGCATTCAAGGAGGGCCTCCCAGAGGAGGTGGCCTTTGTGCTTCAGCCCAAAGGATGAGTTGGGGAATGCTCAGCAGAAAGAGGCAGGTG
AGGGCTGGGCATGGTGGCTTATGCCTGTAATCCAGCACTTTGGGAGGTCAAGGTGGGAGAATTGCTAGAGCCCAGGAATTCAAGAACATCCTGG
GCAACATAGACACCATCTCTGAAAAAAAAAAAAAAAAAAGAGGGCAGGTGAGGTTATCAGGCCTGGGTGGGTGGGAGGCAAAGCCTGGGAGCTC
AGTGGGGGCCTGATCACATCCCAGAGGGCCTTGAATGCCGAATGGAGTTTATCCTTGACTCAGGGTCAACAAGGAAACATTGTGGGGTGAAGAG
TGCTGTCCTCAGATCTGATTTGCAGACCAGTAGCTGCAGTGGCTGCTTGGAGGATGTTCTCACAGGGGTCCCATGATCCCTGCTGCGTTCCAGG
AGAGAGGGCATGAGGCTCATTGCTGGCAGCCGCCTGCGTATGGGCCAAACAGTGCTCCAGATATCATGTGGCTGGTAGGGGCTGAGGATGGTGC
TCATCGGGGGCACTGGGTGCTGTGGTATCTGAAGGGAGTGATTTCCAAGTGCCGTGAGGGTGCCCTTGGCCCTCAGGGCTTTTGCACACAGCAT
CCCTGGGTGCCTGCTAAACGTGCAGATGCTGTTCTCACCCCAGCACAGCTGGGCCAGGTGCATTTAGAAACCTGGCTGAAAACTGCAGGTCTGA
TGAGGAGCACAAAGGTCGGGTCCCTAGAGAACAGGAGCCTTTGAAGGAAGAGGGTGGAGCTGATTGCAGACTGCCGAATCTTGAAGGGGTCCAC
AGAGGGGCTGCAGGGTGTCGACTTGGCTGCAGCAAGGAGAGAGGAGGACCAAGCTCTCCCCTTGATGAGTAATTCAGCATACAGTTGAAGCTCT
TTGCAGTAGGTGTGAGGAAGGCAAGATCTTGGCACTGGGGATGCTACTGATTGTCTAATGGAAGGGGTTCTCAGCACAGGCAAAGGGCTGAGCT
CACTGTGCTGGTGAAGGGGGCAACTGAGTAAGTCCTCTAGGATGAGGGGGAAGTTCCCAGTCTGGAAGTGAGGGAAAAACATCTGAGGCAGAGG
GAACAGCATATGCAAAGGCTCAGAGTGCTGGGGGGATGCTGTCAGAGGGGCACAGCTCTTGAAGCCAGGCCAGCAGGGATGGACTTTGTTCCTG
TCTAGGGGCAGCAAGTCAGCCAGGCTTTCTTCTCTCAAGTCAGAGAAGAAAGGCCAGGGCTACTGGATCCTGGAATGTGTGAGGTCCTTGGCCC
AGTGGATGTCTGGTGGCCTTGGTGTATCTTATTGGCACGGCCACGTTCACAGGACCCGTGGGGTCAGGGGAGGGCCTACTTGTGCAGAGGGAGG
GGTAGTCTCTAGGTAGGCAACTGGCACCTGCCTCCATCTGGGTGAACCAGGGCAGGGAGGGAGGGAGGCGGGTGGCCAGGTGACGGCCGTCCTG
GCAGGTGCGTTCCATCATCTGCCATGAAGACAGCCGTGGGGCCTCAGGGGCTGGAGCTGTTTCTCACACTGTCCCCGACCATAAACACACACAC
TGAGATTCCACCGTTGTCCCTCCCCTCTCTGAGGATGTGATGTGAGGAGCAGCTGTCAATTACCATCAGCTGCCACAATGCAGGGACGCAGCTG
AGGACCAGATAGAGCAGGAGTGGCCAGCTCCAGGTCACACGGCAAGTGGGACCTCCGACCAGCTCCCAGCCTCCCCATGGCACCAGGGACCTCA
CGTGGGTCAGGATCTCTCCACCTTCTCTGTAACTCTGGCTCCTGCTCTCAAGGAGGAAGAGCTGTCCCCTAGCAATAAGCTCTTCCCATCTGGA
GTCACCAGCTTGTTCAGCTTTTCCTCACCTCCCTCCACCTACAAACAACCCAAGGGAACAAACCCAGAAAGCCAGGTCCCAGCCTGTTGGACCA
GGCCTCGCCCAGTTGGAGCAGAAGGCGTGCAGTGGGGAACGCAGCCTGTAGCCCCTCTCACAGGGAGGCAGAAATCCCGGCTCTGGAAGTGTAT
AAACACGGTTATTTTTAGCCTTCAGAGGGCTGTGGGCGCAGCAGCGTGACCTGAATCTGGCTCAGCCCCCTCCTTCTGCCCCACCCGGGAGAGA
AGGCCTCCTTTGCCCCGCCCTCTGCTCCATCAGCCTCAGGGCTGGGCCAGTGAATTCCAGGATCCAGGGAGAGAAAGAAATGGGTGTGTGTGGG
GGTCTCAGCAAGGCTGCCCAGGGCCAGGAGCTGAGGGGGGACAGGATGTGGTTTTCAAAGAGGCAGCATTTCAAGTTGCAACAGAAATATTGGA
GCCACCCAATTTCATGACTTTTCCATGGGTTCTTACTCCCTAATCCTGGTCCCTTCCCTGGTCTGGCTGGAGGCGAGTCCCAGGCCCCACCTGG
GAAGCAGTGCTGGCCTAGCACCAGGCGGCCTTCCTGCTCCTTTGCTGCCGTACGCTACCAGGCCCACATAGCACCTCTCCGCGGCTCAGTGCCC
TCATCTGAGAAGTGGGATCATTCCCAACCTGGCTGGCAGGGAGTTGAGGGGGTGATGGTGAGCTGACAGAATCGTAGCTGTTCACTCACTTGTT
CATTCAACCAACACTTATTAAATGTTAGTTTTATGCCAGCCAGTGATCTAAACATTGGATAGAAAGCAGGAAACCAGCTTGAGGAGGTGCTGAC
CCTCCTGGGGCTAACATCATTGAGCAGGGCTGGGGAACACAGAAGGACAGTAATCAGCTCGTAGTACGTGCAGTACAGTAGTAATGTCGAGCGA
TGTGTTACGGAGCGGCTGGGGTGGGGGAGGCGGCATGTATTTTAGATGAGGGCAGTCTGGGAGGACCCCTTGAGAAACTGTAAAACAGCCAGCC
ACTGTAGGTGGAGGAGGTGGTGAGCGGGAACAGCAGGTGCAAAGGCCCAGGGGTAGGAATATGCTTGGTGCCCGAGAACTGAGGACATGGGGAA
ACCGTGAGGGGCAGCGAGCAGAGCCAGCTGCACGGGGCTCGAGGGCCGTGGGGATGCCTGGGGTGGGGGGCTGTAAACAGGAAAAGCTTTACAG
CTGTAAGGAAGAGCAGGAAGGAAGTGAGAGTGGGAGGCCAAGGAGGGGCTGTTGCCCCCACCCAGGCAAAGGAGCTGGCGAGGAGTGGGCAGAT
CCGCACGCGGGGAGGGTGAACGGTGGAAATGGCAGATGGCTTGGTTGAGGGGGAGCGTCAGAAACCAGGATGGTTCCTTGACTTTTGGATTTGA
TGCCTGGGTGGGTGGGGGTGGAGAGGGCATTTGATCCAAAAGGAGGGCGGAGGGAGAACAGGATCTAGGGGAGGGAGGGCAAGGGGTGATGCCA
ACCTGGACACAGGTTTTCGGTGTCTGTTGACATCGGAGTGGAGAGGGGAGCCCTGGGGGAGCAGCTGCATGGGGCACACCAGGCCCCAGGCTGG
CAAGCCCTCCTCCCTGGGGCTCTTTGCCAGCCCGGAGCCAGGCTGTGTGGGCGAGTGCTWCCCTGGGGGCCTGGCCTGGGGTTTTGTCCCATTG
CATCATGGATGAGGACCTTCCGGTCCCCTGGCTTGGAGGCAGACGTAGGTGGAGGTGCAGAGTGGTCCACGGCTCTGCCCAAAAGGCTGGACAA
GTTGGAAGGTGGTCTGTCATTCAAGCACCATTTGCCATTGGCTGGATGCGAGCCCTGGTGTCACTACCAACCTTGTTCCCAGCTGGGCTGACTT
CTTACTATGAACTAGCAACTATGAACTAGTCCTGCCACTCCTGTGCCTGGACTGGGATGGGAAGGACAGCAGGAGTAGCAACATGCTCTGGAAT
TTTTCACAGCAGACCTGGTTCACTTTCATCTGGTCACCAGACAATGTAGTGACCCCTCCCCTCTCTGGGCCTCAGAATCCCACCTGAGACCCCA
GGATGGAGTAGAAGGTCTTCAGGGTCTCCGTGTGAAACACAGCCTTTCGATGACGGAGATTTGGTATTGAGAGGGTCATGGCGTGAGTAAAGGC
CTGGAGGCTGGTCTAGGGAGTCACCTTGCAGGATCTTGGGGAGCGGATGAGTATAGAGTCCGCGTGCATAGAAGCCATGTGCAGGGTGGTCACT
GGAGGTGAGGTGGGAACACCAGGCTGCCTGGTTGGGTCAGAGGGACCAGATTACCATATAAGCCATTGTGCTCTCAGGATAATCTCCTGGCTTG
TTAAAGCCTCAGTTTTGTCATCTGCAAAATGGGTACAATAGACGTCACTGAGCTTGCAGGGGTGAATACCCACCAAGGTACAGAGATGGGCCAA
CTCAGTGCCTGGTCGTAGGATGTCTTTGATTTCCAGAGGTTGCCTTCTTTCCCTGTCTTTCTCCAGTTTGTAGCCAGAATCCAAATTTCAGGGG
AGGGCTCTTTTGCTGTTGGGGTTCCTGTGAGAGCCACAGCCCTGGGCCTGGGAGTCCTGCCAAACCCAACTCTGGAGGGCCGTGAACCTCACCG
TAGCCTTGGGGACTCGCCCGCTGGCCACGGCACTCTGAGCATCAGCCTTAGGTACTGCTAGTAGGACGTGCACTGGGCTTTTAACCAGAGGCAG
TAATGAGCTTTCTGAAATCATGCCAGCTTCATGTTTTGTTTCTCAAAGAAACGATGGGCCTTGATTTCCTATCAGCATTGATGTGGTTGCTGTT
GTTGTTGGGTTGTTGTTGTGGGGTGTGTGTGTGTGTGTGTGTGTGTGTGTGTGTGTGTGTGTGTGTCTTAACTGCCATGGGTTTTAAAGCCATG
GTGGAAAGTGAGGAAAATAGCAGATGCTAGATCCAATTGCCACCAAACTGTCTTGAAATGCTATAAATATTTTCTAAAAAGACTAGCTTCCCGA
GCCCAATGTGTTAGTTGTGTCCTCCCTGAGAAACTGATTCCCGAAAATTTAGACAAATAACCTCTTAAATCACAGCTGTTAACCTCGGGTGTAA
AAACTGCTACCACACCAGCCGCAGGTCACAGCGACTCTGCTCTGCCTAAGTGCCTCCGTGTGGCATAGAGAAGGTGGTGGCAGAGAAGAGAGAT
CTTGTGCTTAGGCAAGAGACCAGGGTCGGAAGCCGGGCTTGGCCACTGACTGCTGTGTGACCTGGGGCCATTGGCCCACCTCTCTGAGCCTCTT
CTGTGTAACAGACCTGTTACAGGCTGCCTCGATCCTGAGAGTGCCACTGAACTGAGCTTCTTTCTGCTCCTCAAAGGTAAACAAAAGTGACCGT
TGGCATTCAGGAGTTCATGTTAATCAGGACTGAGAAAACCCCTCCGCCCCTAGTCCCCTTTAGCCCTCTGACTGTGTCAAGGACAGCTGCCAGT
GACTGGATACTTGCTGGTGTCTCTTTTCAACAGAGAGCACCTGCAGGACCCGGAGCCTCCAGCAGGCAGGCAGCAAAACAGTGCCATTGTTTGT
TCTCCTTGTATTTGTTTTTGTGTTTAACTTCTATCCTTGGTGAGTAAGGCTAATTTTGTATTTAAGGTAGTAATATAAAGTTCTTTTAAAAATA
AAAGTATTTATGCAAAAGTTGTGAATTGATTATTAAGCTAATGGCAGTTTCGATGGCAGAAGGAAATGACAAACATCAGGAAGATGGGAGGAAA
TGTGACGGACTCTGCACACTGTCCAGGTTGCCCTTCCCCTAAGTGCTCACCAACTTCTGTGCCCTTGTAGAAGGAATGAGTGGGGAGGGGTTGC
TGGAGGGACGTTCTGGTAAATTGTCTTCCAATTGCACAATACCCAGAGGTGACCACCACACTCCCTAAGACGGTGGGGCTACAGAAAGCCCCAT
TCTGGCATCCGGGCCAAGGAAACCACTCTCGTGGAGAGTCTTGGCCAAGAAGCTGGGATTCTGTAGCAAAACCAAATCCCCCCACCCCCAGGCC
CTCTGTGCCTCTCCATTGAAGTTCCCAAACTGAGGGTCTAAGCTTGACACCATCTGGCTGCTCTCTCCTAGGCCAGAGACCAAGACCAGGCCCT
AGCTCTCCCAGATGTGGCTGAACCCAGCAGAATGTAGTAGTTGCTCCACTAACAGGGACATCAAGTGCTGTAGGTTTTGTCTTATGCTCTGCCT
GAGAGTGACAGCCTGGAGTGCTATACAGAATAGCAATTCTGAGGCTACAAGGAGCTCTGTAAGAGAGCGTGGGGACCAATTGGTAATGCCTGCC
ATGGGTGTGGGAAGGAAGCCTGGGGGTACACAGGCCTTTATTTGTGACTCATATGCTCCCCCAAGCCTGTGGCTAGATCCTGCCCCCATTGCAT
CGCCAGCACCCAGCTTGGCGATCAGTGGGTGATTGAGCCCAGAAGCTCCTATAAGAACTTAGCAGATGCTCCTTGGCCGGTCTTTGCCCGTTGT
GCAAGGGGACATCCCCCAACCACATTGCAGGCCCTGGAGGGTCAACACTCAGCCCTTGAGTTTAGGAAGCTCTTGTTCATTCCATACCCACTCC
CTGCTAGGGCTGGATATGGGGTAGGTACCCAGGAAGTGTGTTGATGAGACCCAAGAATGATCGACAAATGGGACTGGAGAGGTGGTTCGGCCTG
GCTTGGCGGCAGTCCATGTAACACTACCTCACTACAGGCATCGTGCAGGTGGCCGAGTGCCCCAGATTTTCTAGGACAGCCCTGGTTTCAGATC
CTTCATCTATTGTTCTTGAGTGCCCCTGCCTGAACAGGCATTTGGGCTCCGCTTTGGAAGTCCTGGCTGCTGTGGATATAGCCCTCATGATCAT
GCTTTTTGATCATTTTCAGAGCACTCACTCTTCCATTGCCTTTGCATCTGGCCAGGGGATGGAGCAGCCCCATTTGCTCACTTTTTTCAGGTGA
GGAAATTGAAGGCCAGAGTGAGCAAGTGACTGGCTTGCGGCCACACAGCCAGTTAATGCAGACATCTGGCCTGAACCTGTGATACCCATCTTCT
GGCCACTTCACTGTCCACTTACAAAGCCCAGAACTTTCTGCATCCTGCTATTTAAGCCTCATTCATTCAGTCAGCTTTTGTAGCCTAGCGGGTT
AGACCTCAGGAGTCAAACAGCATGCTTTGCATTTGAACTACTTCTGGCCGTGTGACTTTTGGTGAGTTCCTTTCTCTCTCTGTGCCTCAGTTTC
TTCATTGATAAAAGGGGTGTAGACACCACACGGGGCTCTTGTGAGTATTACTGGGTTAACAGTGTCTGTCTCAGGGTGAGGGTTCTGTGGGGCC
CGCCAGTGGTGTTCACAGAGGTGGATTGATCAGAGTTGCTCACAGTCTGGTGAATGTGAGGTGGGAGGCAGATGTGTTCACCACGAAAGAGCGT
TCAAGTGGATCGGTGAGGTCAGAGGTCCACTGGQCACCACCGGGCGGTGGGAGGAGGGACCGTCTCCATTGCTCCCACACTACTCGGAGGCATT
CAGAAAGGCTCCTCCGTTGATGGACGCTACTTGGAGCAGGGCTTGCAGGGGGCAGGGAGGGCACTGGCCTGATCCTGGCAGATAGCAACCTCCA
AAACTTCTCTGGACAGAGGATGGCACTCATGCCTACCGCCTCACTACCTCCAGCAGCTGGAGCTTCTGGGCTCTGCTGTAGGGTGGAGGTGGGG
TTGAGAAGAGGTGAAAAGACCCAGTTGCTTTGTCCCAGGGGCAGGCACAGAGTCCTGCCTGAGGGCCCTGGCATGGGGTAATTGTCTATTGTTC
ATCGTCTGGTAAGCCAGCTCCCCACCTTCTTTCTGCCTGCCTGCCTTTCTGTCCTTCCTCTCAGCATCTGTCTATGCAATATAAAGAGCCCCTC
CCAACACAGTCACCAGTGATCTGCTTCCAGGCTAGTGTTGACATCCCAGCCCACCCGGAGACCCATTTATTCTGCCACTGGGGCACAGGGCCCA
GTTTTAATCATCCTCGCTCCACTGTGAGATACAGATGGTTGTATTATTAATAGTCATAATTAATCAGCATCCACAGAGGAGCTTTTCCCTAGGC
AGCAGATCCTCAGGACGAGCAGCCCTGGGAGGTCCCATTCCAGTCCCCCTTTACAGACAGGGGAAGTGAGGCTGGGAGAAGGGGTGGTGGGACT
CATCCGAGGTGGAGCTAGGTATGGACCCCAGCTCAGATCTCAGATCTGATAACCACCTGCAGTGTTACTGTCTGGGAATGGACTGTTCACCCCA
CCCACCCATACCTAGGTCCTAGGGTGCCAATTGCTGGGAGAAGCTTTCAGCACTGGGGATGGGAGGGTGGTGCTCAGGCTGATGGTGCACTTTG
AGATCTCAGGCTGAGAGTGAAGGAAGAAAGGTGGGATGGGGAAGAAAGGCCTGAGGGAGGCCGGACATATTGGGCTAGCAGGACAGGGGGCCTG
ATTGGGGTAAGGGTGGGGAGGTGGCCTTGGGTAGAAGGAAAGCCAAATACCAACCATCACCCCCTAATACACACAGATCGTGGGTCAGGGCCTG
AGGTTGGCTGCTTCTCTGTTCTAAGCTCTGCATCCCAGGGACAGTGATATAAATGCTTACAGCAGGCAGCATCCTGATTTATGGGGCTGTTTTG
GACGGGCCTAACTACATCAGCACAGAGGCAGTGAGGGGCTTCTTGCACAATTCTGCAAGCCCACCTTCTCCCCTGCCTGGCTCCTGGGGCTGAC
CACCACCGAGGCCCCTGTGCATGCATGTGCACATGCATGCACCCAGACCCACGCCTGAGTGGGCACACCCAGGCCCACGTGCTCTGTGTGCACC
TGAGGCTGTCCACATCCCTGCCCACAGCCCATAAACTGTGTTAGCCCAGCACGCAGGTGTGTGCTGCTTTGGGCCTGTGCGCAGATGAACACAG
GTGTGCCCAGGTGCACACATGTGTGTGTCTGCCTGCACTCTGCAGGCATCCATGTGCCTATGTCCTCTGGGCTGCCTCTCTCTGTGTTGATCTG
TTCGTGGCTCTGGGAGGTGGATGTCTGTTCGCTGTCCTGTGTGTGTCCCAGTGTGGATGTGCATTGGTGAGGGGTGTACATGCCTCTGTGCAAG
TGCGCCTGTGTGCATGGGTCTGTGTGCACACATGTGTGGGCATGCAACTGCCGCCTCATCCTGTCTTTTTCATTATGGGTGTTGTCCACACTCA
CATCCTTGCGTGCAACACACACCCGGTGCCCAGCGTGGCTGGGCTGCCCCCGCCTGATCATTAGGCTCATGGATAAGACTCTCTGTCATCGTAA
ATGAGTATAGAGTGCCCCAGTCCAGGAGTCCTGAAGGAACTGGGGAGGTAGCCTCATCTGTCCTCAGCATCTCCACCCCACCTCCATATGGATG
GTGCTGGAGAGGGGAGGCTGGGAGGGAGGGATGTGGTGCCCACTCCCAGGCCCCCACACTGGGTTCCTGATCCACCCTTTGTTTCTCCCCACCA
TGCCTATGCACTCGCCCATATTCTGTGCCAGACCTTGAGGACAGCCCCGGGTCGTGGGAGAAGCCCAGTCAAGAGTCAGGCAGCCGGGTGCACA
AACCCAGGCTCTGTTGCCAGCATGTGGCCCTAGACAGCCCCTCTGCCTCTCCCATCATCACTGTTTTCCCACCTGCAAAATGGTCTTAGCAATG
GCCACCTCATGGGGTTATTGTGAGGACAAAAGGGGTGCAGAAACTGCTAGAGAAGTCAAGTTCCTTCCCCCAGCAGACCTCAGTCTCCCCATCT
GTAAGATGGCAATAATGACCCTCACCAGTGGTCTCATGGAGTGTTTTGAAGGTCAGATGAGAAAAAGGTTCAGTGAAGGCTGCAGGGTGGTACC
CATAGGACTTGTTCCAGCTCTGAGCAAAGGAGATGAGGGGTGCTAGGCACTTGGGGGTGGCATGCATGGCCTGGGCCAGATGCCTGCATCCAAA
CCCCCATGCTGCCACCCTCTGGCCTTGGGATGTGGGAAGGTTACTTCATCCTCATCTGCATCTCTATTATCCTTAGATTCCTCATCTTTCCAGC
AGGCATGTTCGTAGTAGTATCCATCTCCTGGCATGTTGGGAGAATTCAGGAGAAATGAGTGTGGAGCTTAGCACAGAGCTTGACACAAAGCAGC
TGCTCAAATCAGCGGTATTTGTTATTCTTGTTTTTGGCCTTATTGCGATACCGGTGGCCACTCGGTCTGGAAGGACAGGTGAATGCATGGCGGA
TGGTGCATTAATACCACACTAGGATACCTATTAATGAGGTTTAATCTGTGTTTCCAGACTTGATGTCCATCCGTCCCACCGAAGAAGAGTTCAA
GCTTTTCTTCCAGGGAACTGAGAGGGCCATGCTGAAACTGGAAGCCTCAGCCTGCAAGAGGTGCCCTGGGGAAGGAGGTAGTTGGTACTGATGT
TGGCCGACTACAAAGGAGCTGGCTCCTGGGACTCTGAATGCTGTGCCCTCTGCAGCCACCCAGCCCCACAATGGGAAGCCAGTGGTTACATTTA
CCCCTGTCTCTTCTGAACCATATGGTGGCTGCACCACAGCTGGCTCAGAGGCTCTGAAAGAAGGCCCACAGGGGAACATGATGTCTTGCCTGAG
CCAGGTGAGGACTGAGGCCCCCACTGCCAAGCACTGCAGTCTGGGGCTTCCATTTTCACCATAGTTCCTCTAAGCTGCCTGGAGGAAGGGCTTG
AACTCATGAACTTCATGACCAATGGACCTGAAGTCTGGAAGCACAGATGATACCTCATTAACAGGTATCCCAGTGTGATATCAATACATCATCT
GTCATGCACTCACCTGTGCTTCCAGATGGGGTGGCCACTGGCACCGAGGGACTCCTTGCACAGTTATGCAGATGGCTGCCAATCTACTTGCTCC
CCTTGCCCAGCTCCTGGGCTGACCACCACCGGGGCCCCTGTGCATAGAGCCCATGAGCAGTCCCCATCACCTGTGGGCTTCCCAGTCTGTGCCC
AACTTAATGATAGCTGTTAGGGTCCAGTAGAGGATGCAATGTCCCCATCCTAGCAGGGGCTTTACTGAAGAAACGAGGCCCTGGCACCCAGGCC
AGGGCAGGGAGGAGACAGTTCGTAGACACAGCCGGGTCTGCAGGGGCTCGGAGGTCATGGGGCCCAGGTGGGCAGGCAAGGGAGGAGGCAGGCC
AGGCTGGAGCCAGTCCTTGAAGGACAAAGAGGATTTAGCTCACTGGAAGGGATGGCATTCCAGAAGGACAGAAGTGTGTGGGCAAAAGTTTGGA
ATCATTCCCGGGGAAAGGTGTACCCTCTCCCATAAGAAGTTTGGCCTGCCCTGGACCAAAGAGCAGAGTCTTTGCTCTCGATGGCTAGTGGCTG
TGGGGTGAAGCAGATGGCCAGGGGGTGTGGGTGACGGGGGAGGCGCACCCTCAGGCCTGAGCCAAGGGAAACCAAGTGGGCCCACACAGGCCAG
AGCCTGAGACCTTGGCACCAGCTACTCGGAGATCTTGACTCAAGAGTTAATGATTCATTTGTGGCCAATTTTCTGCTGGCTTTGCCTGGAAATT
CCCAAGCTTGCCCCTTCCCTCACCTTAGGGAGCTCCAAGAATACAGTCTCACTTACACTTGGCTGAGAACGTCTCACCCACTATCCAGGGAATG
GTTTTTAAAATTACAGCAGGTGTGATGTAGGTTAGCCATTAGGAAAAGCTGATCGGGGGCTATAGGCTGGGAGCTGGGGTGGTTTGTTTGAGTG
TTTGGAACAAATGTGAATAGAATTCAGTCTAGTGTAAAGTGTTGTGACAGTTACAAATTGGGAGCTACAGCTTGACCCAGAGTAGGACTTGGTT
GGGCTAAAAAAGTAATGGCATAGGCAAAGGCCCTGGGGCATGAACATCCAGGGAACGCTGAGTGATTGTGGTGTGGCTGGAGTGTAGAGGGTGT
GAGAGACTTCTAGAAGTGTGGACACAACTCCCAAGTGAAGGACTCTCAGGACTTGAAGATTTGGCAGTGGGACGTGCTGGCAGTGGGGAGTCAG
GGCTGGTACCTGAGTAGGGGACAGGTGTGTTTGATTTGGGTTGTAGGGTCGCTTGCCCATCCCTGTCTTGATTCTGCTGGTCAGCAGACCTTCC
TTGACAGCCTGCTCTGTGCCAGGTCGGACCCACGCTCTGCACGGGTCCTGCACCTGTGTGGAGGCTGAACTAAAGGGAGGGGGTTACATGTAGC
AATTCAGGTGGGAGCATGGGTGTGTGCAGGAGCAGGGGAATGGCTGATGACTTCAGACCCACTCGGCTCCATCACCGAAAGTCCTTGGGTGAGC
GTAGCAGAGGTGTTGGGGTAGGCTTCAGGAACCCCATGTTCCTGGCAGGCTCTGTGGACACGCCGCCTGGAAACCACTTCTGCCCTGTAATATC
AGCATGGCTGAAGGGGCCTGGAATGTGTATCTGTCCTTGTACAGAGGAGGGGAGCAGACGAGGCCCAGAGAGGGGAAGGTTGATGGCAGAGCCC
AGGCAGAACTCAGGGCCCAGTCTCCCAATCCTCGCTGATTGCACATATATCCCACCACCCACCCGTTCCTGCAGATCTAAGGACATTTCCCCGA
AACCAGGCAAAATAGAGCAGCCTGATTGTCCATGTCAAAATGTCTGGGCATTTGGGGAGGGAGGGTTCCTTTATATCTTTCACAACTTAGCAAA
TTTAATTTCTTCTGATTATTTCTTGACGGTCCCAGGCGTTGGTGCTAAACAACCACACTCCCCAGAATGTTTCCCTGTGACAGCAGCTTTCTCT
GGCGAGGGTGCTATCAACACTTGGCAGGGCCTCCTCCTGGCTCCTGGCTCCCAGCTCCTGAGATCGTGAGCTCCTGAGCTCCTGGGCTCTTGAG
CAAGGCAGCAAGTGAGGGCTTCTCAACAGCTTTCAGCATGCAGAGCTTGCCCTGTGTACCAGGGCAAGGACAGGCCTAGCCTCTTTGTTCCCCG
CAGCAGCCTGCCCTGCCTTCGGAGCCTCAGGTGGCCTCTGGTTGTGGATGGTCCCCATGCAGCGAGGCTGGGTGGTATCAGATGTCTGCTGCCT
CCCACAGCTCTGGTGCCGAGGTCCTTGGCATGGCTCAGTGGCAGGAGGGAGCCTGTCTTCTGAGCAGCTCTGCCAGGACTGTCTGTGGCCGAGA
GACCGGCAGCATCCTGGGCCTGCAGCCTCCATGGTCTGCGTCATACTGGCTTAGGTGTCCTGAGTCAGAGCCAGGAAATGGATCTGCCATGCGG
GGGTGGAGGTGCTGCTGCCCGGGCAGCTAGGGTGAACCCCAACCTCCTGCCCGTCCACACCTGGAGGGTCCTGGCCTTGTGCTTCCTTCCCCTG
GCCTGGCCTGAATGTGACTGGTGCCTGGAGGAGAGGCTGAAGTTCCAGCCTGCTCCACACTCCACTTTATGTGCCCAGGCCCCCACCCCACAGG
CTGTTGTTGGCAGGGTGGGTGCTGAGACACAGGCACCACGCTATGCTGTGCTTTTCCCTGAGTTATTCTATTCCCTTTTTCTAGAATGCCCTTT
CCAACTCGTTGTCTGTGTGATTTTCTGCTCCCCCTCCAAGAATTTACACAAGAATGACTTTTTTGGTGAAGGCTTCCCTGGCACCCCCACTCTG
GCCAAGGCTGCCCCCCTTTCCTCTGAGCCCCCTCTCCATTGATGGCTCCTATCACAGTGTAGTATATTCACGTTTGGAGCCCGTTCACCTGTGT
AGGTGCCTTGAGGCTTTTTTCTTTGTGGGTCCTACAGGCCTGATGTGTAGTATCAGCTCAATAAAAGCAGGCTGGATTTAATTAAGGGGGTTCC
TGAAGGCGGTGCTGGAGTCTCCCTTTGTTAATGGTGTGGTGAAGAGGAGGCGGGAGTTTGCAAATGGAGAGAGGCTGACTTGCCTGCCGGACTC
CTACCCCCGATCCCCTCTGAGGGCAGTGCTGCCCCTCTGACTGCTGGGGACTGGGGIGGCCCCCTCCATTTCTATCCAGCGGCAGACTTCCCTG
CCCGGCAGGAGCTGGCTCCCCAGGTTGCCGGAGTGATCACTCTCCAGAAAACACCCCAGTGGAAACTGCATGAATGATTGATAAGAGCTGAGAG
CTGTTTAATTTTTTCCCCCTGTTTAATGGCTATCAATAATTTAATACGCTCTCTGTATGTTTTTAAGAAATAGCACCCATTTCGCCGTCTCCGG
TTTAGCGGGCTAAATGATTTAAGTGTTGGAAATGTGCTCTGAGGATGGAGGTTGAGTGAGTCGGAGAAATCTGGAGGCTGGGAGTGGGGGAAGG
CGGGTTCAGAGCCAGCCACAGCATCTTGGCCTGGGTGTGTATGTGTGTGTGTGTCTGTGTGTGTGTGTGTGCGTGTGTGTGTGCGTGTGTGTGT
GTGTGTGTGTGTGTGTGTGTGTGTGTGTGAAGGTGGGAGGGAGAAGGGAGGGAGGGAAGGGGCGGGTAAAAGCCAGCTCCTCTCTGCTAAGAGC
AAGATAATTGCTTTTCCGGGGCCTCACTGAGCAGCAGCTGAACAGCTGTCAGCACCCCCCTCCACAAATGCTAAGCTTTTTTTCTGGATGGCTC
TGGGGGGCCCAGGGGGGCTAGGAAGGAGCTGCCACCAGTATCACCACGGGCTGAGAATCTCCCCTTCCCCTGACATCTTTGCCCCCTCTTAAAG
CTTCTAAATCACTTTTATTTTAACGACGCAGTAAGGCTGCTGCCCGGGTGGTGTCTTCCAGGTGGGGTGTGGGTGGTGGAGTGGAGTGAGGGAC
TCAGAGGGACCTGGAGGGTCATCTGCCAGTGCTCCCCCAAACTTTCCAGATGCAGAGCATCACCTGGGAGGACTTGCTAAAAATTTAAATTTCT
GGGCTGTGTTCCAGACCCATAGACTTCCACTCCCAGGGGAAAGGCCTGGGAAGTTGTGGGTTTAACAATCAGCCCAGGTGATTCTTAGCAGAGG
AGTTTGGGAACTATAGAGTCCAGCTCTGTTCTGGTGCTTACAGCACCCACAGCACCACGTGGCAGCTCCCTTCTGCTTGCATGCCTCTGGGAAT
GAGGTACTCACCACCTCCCAAGGTAGCAGACCCACTTGTGGAAGGCATACTCTAGTCTAGAGTTGCTTCTGATGTTAGGCTGTGACCTACCCAG
CTGCCCTTGTGGGCTTGGAGTTCTCTCTGCTACAGGACAGACCGTGTTTCCTGGGATCTGATGTCTTTCCCTCTCCCAGCCTTCCCACTTTTTC
GCTTGTTTGGGCCTCTCTGCTGGGTAAGGAGCTTCCTCTCTTAGCAAAGGGTCCTGGAGGTAGAAGGCAGGAAGGTGACAGGTGCTGTGGCCTG
TCTGTAGTGTGTGAGGTGGCTGCATCATCCTATTGCTGGTAAGCATCATCGCTGGTACAGTGGAGACCCTGAGGAAGGCAGAAAGATGGTGGGC
CTCAGCACTGCCTTGGCCTGTCAGGGATTCCATTCCTCCCACGAGAGAGAAAACTGGCTCATCAGAGATCGTTGTGTCAGCCATAGCTCTTAGA
TGACACTGGGGGGAACTGAGGGCTGAGGGGTTTCCCCAGAGACCACATATCTATTTAGGACAGAGCTGGAGCTGCAAATGTGGGGGCGAGAGGC
CCTTAGAGGGGTGGCCAGGCCAAGGCCCTGGCTTTAGGGATCAGCTGAGGCCCCAGCCAGTCACTCACCTGAGGTTGGATGGGGGAGGGCTTCC
GCCTTCTCAGTCACTCAGGGCTGATGGACAATTCATGGAGTCAATACATGGAAGGATTTTGGTGGAGCCGATGCCCACTCTGTCTGGCATTACA
ACTCTTCCTTCTGTCTGCCTAGCAGTTTCCATTGTTTTGCCAGAGGGTGTAGCATTATACTACGGCCATGGGTCTTGCAATTTTGTTTTAATAG
ATAATTAAGAGGGAAGGAGTGGTGCACATTTACTGGGCATCATGGCTTACTGTGAAATGGGGAGTATAATCCCCCCGTAAGCCCTGCACTCTGT
AGCCCCTTACTGCACCCCCGGATTCAGCCCCACCCTCCATCTCTGTGGGAGAAGCCTGGGTCCCCAGGAGAGAATTGCCCTGTTGGGTGCTGGC
AGCTGACCTATCTTGAGATCCCAGTTCCCCATCCTACCCTGAAGCCTGCTCTGGCCATCACAGTATGGCTGGGGGTGTCTCGACCCTGGGGGAA
GAGGAGTAGGGGTGTCACCCAACCCTGATAGAGCTTCTGCTTGGAGAACTCCGAGAGCTGCAGAGGCAGAAGAGAGACTTACACACTCTGGGGA
AGACCCAGCCCCAGAGAGAGAACTGTGTGAATATGGACACCCAGGCAAGGCCTGGGGAGGTCAGGGAGGGCCTCCAGGAGAAGGTGCCACCTGA
CTGCAGGCTTGAGCATGGTAGGAAGGGGAAGGGCATCCTGCGGTGGAGGTGTGATGCAGCCTGTGGGGGTGCACAGAAGAGTGAGGTAGAGCCT
GCAGGGCTGGGGATGGGCTACAGAGGCCGGCTGGCCAGGGCGGGGCTGACCTGATTCAGTTGGGTGGAAAGCCACGGAAAGATTTGTCTTTAGT
TCCATTGAAGTGTTACACCCATGCAGAAAAGTGTGTATCTCCTACATTTGTAGCTTGACAAATTATCCTGAAGGGCACATGCTTCTGTAGCTGC
CAGGCAGATCAAGAAACAGAACCTTGCAGACCCACTAGAGCCCCCTGTGCCCTCTACTCACTGCCCCCTCAAGGATACCGACTGTCCTGACTTC
CAGCCTGCTGTGAGTTATCTCCTGCTGCCCAGCAAACCACCTGAAAACTTGGTGGCTTAAAGCAGCCACCATTTATGAAACCTCACAATTCTGT
GGTTTGGCTGGACTTCTCCTCCTGATTCGCTTGGGCTCATGTGACTCCAGTCACGTTTGAGTGTCTGCTGGACTGGAAGGTCCAGATGGCCTCA
GTCACACGGGCAGTGGGTGCTGGCTGTTGCCAGGGGAGTTCCCCACCCTCCACTAAGACAGACACCTCCCTCCATGGCCTGCAGCCTCACAAGA
GAGGCAAGGCCAGAAATGGCAGGACCTCTTGAGGAGGCCTAGTCTCTATAACTCACAAAATGTCACTCACACCGAATCCTATTGATCTAAACAA
CTCTCAAGGCCAGCTCTCAGCAGGTGGAGAACAGACTCCGCCTCTTGATAGGAGGAACTCCAGAGGACCCCTGCCGTATTTCATCTATTACAGG
TGTAGAGTCTTTTTGCCCTTCTTGACCTTTATATAAATGGATCTCATAAGTGTATTCTCTGTCGTGCCTGGCTTTTTGAGTCAACATTGTGTTT
GCAAGATTTACCCGTAGTGTTGTTGGATGAAGTTGCAGATCACTCATTCCCAAGCCTGTGCAGCAGTTTGTTGTGTGGAAACACCAGTTGACCC
ATTCTATGGTTGGTGGCCATCTGGGAAGTTTTCATTTTCTGGCTGTTACGAATTGCCTTGCTACGAGCATTCTAACACATGTCTTCTGGTGAAC
ATATGCACATTTTTCTGTGGACATGGAGTTCCTGGGTCATGGGAGAGGTCACAGGGTAAGAGACCCTTCCAAACTATTCTTCCAAGTGGTGGGT
CTAGTGTCTAGTGTGTGCCCCACCAGCAGAAGGGGGACTTTACAAAGACCCAGCTGAAGATGGGCTGGAGGTGCATCTTGTGGACACTGGAAGG
GGCTTCCTGTGATGTCCCCTCTGCCCGCCCTGAGCCTCAGGCCCCCAGGTCAGGGGAAGTAGAGCCATGAGGCTGATCCCTCTTTTCTGAGCAT
GAGCTTTCCTGCTACACTTTTTCCACCCCAGAGGCCTCCTATTGCCTTCTCCTTTTCCCAGCAGCTGCCTTTCATGGTTGACCTGACCCTCAAC
AGTCAGGGAGGCCAGGGGTCTGCAGATGCAGGAACTGGGGGGAGACAGCCACACAGCCTCCATCCCTGAGGAGATTTGGACTTCACTCCAGCCT
TGCCTTTCACCATGGGAGGAACCCTTTCCCTCTCAGGGCCTCAGTTTCCTCATTTGTAAAATGACAAGGAAAAGTATCAACCTCACCAAGTCCC
TTTCTTCTCATCAATCTGACCCCATATTCTAGAATCGAATAGAGGAAGGGATGGGGGGAGGCCTCCTGACTTCAGTCAGCCCATGTTTTTCTGT
TTGTTCACTGCACACTTTCACGGCACCATTCATCCATCAAGGAATCTCTGTGGTCCTTGTTAACCAGTTCAGAGGACAGAGAGAAACTAGACTT
CACTCCCAATGTCCAGGAGCTGGGCCTGGAGCGGGAGAGTGTCCTGTGGGAAGCGGGAGGAGGCAAGGTTGGCCAGGCGCACAGCCCACTGGAG
ATGAACAAGCAAGGAGGGGCTGGTGAGAGAGGGCTCCACGGAAGCACTTGAGCAGAGTCTCAAAGGCCAGGGGCCCTTGAAAGACAGAGAAGTG
GGGAGAAGAGCTCTGAGCGGGGCACCCAGCGTGTGCCAAGGCCGGAGCCAGGGGCTCTGGGAGGCCGGGCTGACTGGAGCCTAGTGGTGGGCAG
GCAGGTTTGGCTGCTGCTGTGGTCTGGCTTTCAGGGCTGCGGGGCTGCTCGGCCGGTGCTGGGGCTTCGAATGTGACGTTCAGGAGCTGCGACC
TTTATTTGGCAGTGGCAGGACGGTATCCAGTGTCTCGAGTAGCTCCACGTTGGTGGGCCAGTTCCATCTTCCAGGAGATGGCAGCCAGGACCTA
GAGGTCCATGACGTGGGTGAGGGGCTGCCCGCAGGGTCAGAGCTTGGAGCTCACTGCCCCCTTTCTTGTCTCCCATACGACTCACGGCAGCTGT
GTTCTGATTTCGTACTACTGCTGGGGCTGCCACCTCCTCCTCCAGACGCTCTCAGCAGACTTGAGTCCTGGTCCTTCTGCAGAGGCCTGAGCAG
GTAAGTGGGATGGGCTGGCCCTCTGGTGCTGTGCACCTGCTGACGTGGGGCAGACAGGGAGTGCGGGCTGCCCAGAGTGCATGGTGCTGTTTCA
GGGGCCCAGAGGGCTGGGGAGGGGAAGGGGCTTCTTTCTCAGTCTTTATTTGTTCCTTGGCCTTTTCCTCTCAAAGAAAGCCACACAGTGGAGG
GTGGTGAATGTCCTGACTTGGGACCTGGACATGGGATTTTATTCTGCTGCCAACTCCCAGTATCGCCTTGGGTAGCCCTTTCCTTCTAGAGGGC
CTCAGCTTTCTACTCTAGTCCATGGAAATAACAGTAATGATATCCTCTAACCCGACAAGCACTTAACTGTATTCTGGGTACTGTGCAAAGTACT
ATAGAGTTATTACTTCTTTCAGCATCACAGCAGCCTTATGAGAGCATTGCTGTAATCACCAGCATTGTACATGTGAGAAAACTGAACGTCACTC
ACTCAGGAAGTAACTTGCCCCAGGCGGCACACCTGGCTGAGCCTAGGGTTCAAGTCCAGGTCTGTGTGGCTCCAGTGCGCCAGCCACTGCCCGC
TGGCCTACTCTGCCTCTGGCCTTGCTCCTAGCTTCTTTTTGGGGGTGGGGATCAGGGCAATTATGGGTGTCTTCAGAAGTGCCAGATCCTGTCC
CAACTTAAGAAAAAGGGAGACGGGCTTTTACCAGGTCACAGGCTCCTGGTGGGTCTGGGGTCATGACCCATTTGTGATGTGATGTGGCAGGGAC
CAGGCCTGGCCTCTGGGTGCCTCTGAGGTGGCAGCCAGGGCCCCCTCTAGAGGAGGGGTGTGTAGGCTGGATAAGGCTTTCTTTCCCTCCGCCC
CTCAAGTTCGTTTTCCTAAGCTTGTATTAAACACTTACTGTGTGCCAACTCCTGGATTAGGTACCAACTGCGGTGCAAGGGAAAGGGGCCTGTC
CCACTCTCTGCTCCGTGGAGAAACAGAAAGATCCCAGTGATGTGGCAAGCCCCATAACAGTGGCGTGGAGAGGGTTCAGAGGCTGGGGGGAGAA
TTCTGGGGAGGTGACCACAAGTGAGTTACAGTGTGGGGAGACAGAAGCATAGATGGAGCAGAACCTGGCAGGATGTGCATGCACACACAGACAT
GAGTGGTGGTGAGTGCATAGATAATACAGATGTGCAAATGTGCGCTTCCTCACAAACGTGTACACAGACGTCGCTTCTGCATGCCACATGAATG
CACACAGATAACACTTGCGTGCACTTGCATGCTCACATACACATGCACAAACTCGTGTTGGAACTACACACATACACGGGCACAAACTCTCACA
AGCACACAGCATTCTGACACACGGGGCTGCATATCCATGCACACACAACGCAGGGTGCATTCCAACAAGAAGAGGCAGTTCAGAGGTCGATGGC
TCTGCATCGGCAAAAGCTTCCAGCAGCTCCTGGGAGGCTCTGGACAGCCTGGGCCGAGGCTGAGCTGGAGCTCTGTTTGTGTGTATGTTGGGGA
GGGTGTTGGGGCTTGAGGGCCAGCCATGTGACTGACCATGGACAAGCCCTTGCCCGCTCTAGGACTCATTCCCCATCTGGAAACAGGAATCGTC
CCTGTCCGCCTCCTATACGGTGGCCGCAAGGATGAGAGGCTGCTGTGTAGATTGCCGAGAGCAATTTTGGGCACTCTGCAGGGCTTCAACAAAT
TGAAGCTAGTAGTCTCATGAGAATAAAACAGGTTACAGGTAAAGTGGGGATGTGGGGGCTGTTCCTCTCCAGGCTTCTCATCCCCTGGGGCCTG
GGTACTCTCTGAATCATGGGCATGACAGTCCACCCTGCCTGCCTCCACCAAGGTGGTGGGGTCTGTGTCATCCCTGCTCCCGCTCTCCCACCTG
CACTTCCCTGTTGACTCGTGAGGAGTTGGGGGCCTGCAGTGCCCAGCTTTGGTACAACAACCACATTTGACTCCCTGCCAGGAGACTGGCACAT
TGCCTCCATGAGTGCTCTAAATCCCCTTGAGAAGCCTGAGAAGGCCTTACCAAGCAATTTCCAGATGAGGAAACTGAGGCACAATAAATGCAGG
TGAGGTTCTCAGGGACACCTGCTCATAAGTGTTGGAGCTGGGCTTCGGATCCACGTCATCAGATGTGAACATTTATGCTCTTTTCTCCCATGAT
CTCCTCTCAGGGATAGGTCATTTATTACCCACTTTCCAGATAAGGAAACTGAGGCTCAGAGAGCGCACGCCACTCAGCCAGGAAGTAGCACAGC
CAGAATTGGGTGCTTATTAACCACACTGCACCCACCTCAATTCAACACTTCCTTTTCCTTTCCCTCCCTCCTTCCCAGAGAGCCCCTCCAGGTT
AGAGGGGGTTCCCGAGGAGGCTTCTGGGATTATCTGGGCTTTAGTTCTGACCTAGTATTCCCAGGCATACCTGCCATTTTCTTGCAAATAGAAA
GATAGGCTATTCTCTGGGGTCTGCTTTCTAGCATGACCTAGTAACTTCCTCTCCACAACTCCTTTGTAAAGTATCCTTAGGATTGTCCTTTGGA
AACTTGGCCAGCAGAAAGCTCTGGGGTACCCAAGAGGCTTGGAGGAGTCCTAGGCCTTTGGAGAGGTAGCCACTGCTTGCATATCCACTGGGAG
GCTCTGTGACCTGCTAAAGGATCAGGGTGTGGGACCACAGCACAGGGAGCCAAGGTTCTGCTTGGCATGGGTGGTAGGTACCACTATGGAAGGA
AATTGGGAAGGATCCCCGGGAGGTGATATTTACGCTGGGCCTTGGAAAGAGGAGAAATTTGCTAGGTTGATGTCAAAGGCCAAGGAAGACCCAG
CAGAAGGCTCAGCTCCACCAAAGTCCCAGAAAAGTGAGAGTTTATTCATGGCCCTTCGCACCTGCATCTGTACCCAACAATACCCATCAGAGTC
TCCTAGAATTAGGGCCAGCTAAATAATTTAGCTGTGGTCCAATGCTAAATGAAAATGCAGAGCCCTTTGTTCATGAATTACAAAGGATTTCAAG
ATGATGACAGCAGAGTAGGGTCCTTCTAAGCTTGTGGCCCTGTGTGACGGCCAGTGAAGCCAACCCTGCTTGAGTAAGTGTAGCAATTGTGGGA
TTTAGCGCCAAGAACTGAGTTCACGTGCAGGCGCCACCAGTTACTTACCAGGCCCACGACCTTGGGCAAGTCCTCCTCATCTCCGTAAGCCTCA
TCTGCCAATCAGCGATGGTACCCTCCTGCCTGCAGAGGAGGGTCAGTGGTGCCAGCATCTAGCTTATCCCAAGCAGTCCAGGACAGAAGCTTTG
GCCGGGGTGGAGTGGCAACTCTTTCCGTCACGTAGTGATGTTCAAGGCTCTCCTGACCCACCCCATTTACCTTCCAGCGCTGCCCCTGTCTACC
TTGGTGGCCTGGTTTTTTGTGCTTTTCCAAATCCACCTGGCTGGGCAGTGGAGGGTATGGGGAGTCTACTCTGTTTTATTTGCAGAGCCTCCTG
TCTGGACTGCTCCTTGCCCACACTCCCATCTCTCCTTGGCAGACTCACTCCTGCAGGTTAGATGTCATGGAGATTTCTTTGACCCCCCAGCTCC
TCCCTTCCAACCTTTACTCTGTGATACCTTCAGTAGCTGCAATGACCTCTTGCACTATAGAGTTCTCTACAGTGTGCGAAGTCCTTTCACACCC
ATCACACCCTGGAGAGCTCTTGAAAGGCCCTATTCCCATTTGCTAGATAGGAAAACAGAGGCTCAGAGGTACAACCATCCTAAGTTCCTGGGCT
GAGGTCAGACCCACATCGGGTAGGGGTGTAGACAGTGGCAGCAGAGAATGGTACCTAGGCTCCACCCTCGGCCGCCTCATCTTGCAGCATTTGG
GAAAACTGGCTTTTCCCTTCTGGTAGAAAGCTCCAGCCCCTGGCCCAGGAGAGGTATGTGGGTTGCTGAAGAGGGCCTGGTAGAGCGCAGAGGT
CCAGCCAGCTAGAGCAGACTAGCGCCCAGCCCTGCTTTTCCCCACAAGTGATGTGGCCCAGTTCACGTGGCTCGGCGGACTAGAGCCAGGTATA
CATGGCTCTGAAGTCTGGGGCAGAGGTGAGAAGGCCCTAGCTGGCCTGGGCTGCAACCGCAGGCAAAGGCTGGCCACAGATGAGAGGGGTATCG
TGTCAGGGGCATGACAAACTGTTCCATATGGCTGGCACTTCACAAAAGACACTCAAAACACTCTTGGTGGATGAGTGTGGAGGATGCTGGCAGA
TTGCGGCTGTGGACCGGGCTGAGGCATGGAGGTTTTCCAGCTCTGTTACCCCTTTCTTTGTGGCTTCTGATGAGCGACTTCATCTCTCTGTGCC
CCATTTCCTCATCTGTAAAATGGGGAGAATATCCCTCTAGTTGGGAGGGATTCAACGAAGTAAGCCATGGGCACTCTCACAGCCATGCCTGGAG
CATGGTTAGTGCTTTCTGGCACTTTCTAGCATGAAGAGTGGAGGTGGCAGTAGCGGGAGTGAGTAGGGGGTGGACTCCAGGGCTGTGGCCACAC
AGGAGGGTTTCCTCCACTGAGCAGCATGGTTGTGCGGTCTTTTAAGAGAAGGTGGAGAGGGAGATCAGGGATTGGGGTTGGGCTGGATTTGGAT
TTCATATGAGGCAATCCCAAACAGCTGTTGCTCAGAGCTCTGTGGGCAGCCACCTGGGGTGAGGGCTGCACTCTCCTCGCCAGCCCCTCCAGCC
CCTGGTGTGTGGCTTTAGGCTGCCGGCATGCACACCCTAAGTTGTCAGTCAGCAGACTTCCAGGGTTACATAGGCGGTTTGGGGCTGGCCATGA
AGACCTTGAATGCCAAGCCCAGCTCACAGCAGGCCTGGCCAGGGCCCAGCCCGTGGCCACACGGAGCTGGGATTCCAGGGTCCGCATATGGCAT
GCCTGTCAATCTTCAGCGCCAGCTGAGAATGCACACCTCTCCTCCCACTGCGTGAAATCTGTGGCCTGCAGCCCAGGCCCTCCACCCCAGCCTC
TGCACCCTGCTATCACTCCACAACCCCCACCCAAGCCTGGATCTGGGAATCGTTTGCAGCTCACTGCTTATTCCTGCCCAAGGTAGGATGCCCC
TCTAGGTCAGGAGGGTGGGACTGCTGTCCTGGTGGGCCCTCTGAGCAGGACTTGTGTTCAAGCCTCAGTCCCATTAAGAACTGCTGTGTGACCT
TGGACAAGTTGCTGCCCCTCTCTGAGCAGCTGTTTATTGGTCTGTAAAATCAGAGGATTGGGGTAGGTCAGGGATTACAAACTGCAGTCTTCAA
GGGTGTCAACAACCCATTGAAGTTGGGACTATCCTGTTGTACAAATGAGAAAACTCAAAGCACAGAGAGGTTAAGAAACTTAGCCAAGGTCACA
CAGGTTATAGTGGAGCTAGGACTCAGCCCAGTTTTATCTGACACCATAATATGTGTTTTCCCTACCACAACAAACTGCCTGTCACAGATACTTG
CTTGTTTGCTGGGTGATCCATTTTGTCCATCAGCCAGCTTGGTGTTGAACACTTCCTTGTGCTCCTGCACCGTGCTAGGCCCTGGTATCCAATA
GTGAATCTTCAGTTGAGCTGGGAGAGCACAGCTCCAGGTCCGGAGTTCTGAGTAGAGGTCACAGTCTCCAGTGCAGGGTTGCTAGGTAGGTGCA
GCTCTTTCCTCATGCTGTAGGAGGCAGCCAGAGAAGACTTCCTGGTGGAGGGGTAAGGGGAGTGGGGTTCCTATACAGAGTGGAAGAGTAGGGT
CTTCCTGGGGTCTCTGCACAACTGGGTGGCATGGGTCAGGGCAGTCCGGCTGGGGAGGAAGATTCATTCAGCCACAGCTGAGGCAGCCCCTGAG
GGCCTTGAATGCTAGGCCACAGAGTTTGGCCATGAACCTGCAGGGAGTGGGGAGCTAAAAGAGACTGCAGGGCAGTGGCACCTCCTTTGTGAGA
GCTAGGAGGGCTGGCCTCAGCTTGCAAGCTAGGGCAAGGGTGAGGTGTGGTCCAGTCTAAGACTTAGTTCACCCAGGAACTGTGGCAGCAGGAA
GAGCAAGGTTGGAGGCTTGTCCACTGGCCGGACCTTCATGCTCCACCACGGCCTCTTCAGCAACCCAGGCACCTCTGCTGGACCAGGCGCTGGG
GCTTTCTACCAAAAGGGAAACCCAGTCTTCTCAAATGCTCCAAGATAAAGCGACCGACGATGGAGCCAGGTGCCACAATCTGCACACCTCCCCC
AACCCCATCTGCCATGGGCCTGACCTCAGCTCAGAGCTTCGGGCAGGCGTTGCTCAGTGACACAGTCGGATCAGAGAGGCTCAGAGACCAGCCC
GGGGGCACACAGCATTCCTCATGGTGGGTACAGAGGCTAGAGCAGGTAGGCAGCATCTTTCCCAAGAGAGGTAGGTCTGGGTCTCCCTCTGGCC
GTCCCAGGGTCCTGGACATGCTTCATGGATGGTGTGTGCTCACTCTGGTAGGAGTGTAAGGTTGTAAAGAGTTTCCTGGGTGTTACTTACTGAG
GGCGCTTCTCCCTCCCACTGATGTTTGCCCCACTGGCTCTTTTTGAGCTCTGGAGTTTGTCTCTTPCTGGTTCATGAGCCACACACCCAGGCCA
CACATTGGGGCAGAGGACAGCCTTGGCCGTGCCTGTGTTCCTAGGTCTGAAGCCTCAGCGACCTTGCAGCAAGTAATAGACACTGGGTGGGGCC
ACAAGCGTGACATCTTTATTGGAATAAAAACAAAACCACAGCCTCTCTCTCGTGTTGGGAGGAGTGGTCTTTAGAAATCATTTTACCCACCCCA
CTCATGGCACAGATGAGGAAACTGAGGCCCAGAGATAGGAGGGCATGCCCCAAGGTAGCCAACATGGCGCTTCAGGCTATCAGCTGAGTGCAGG
GCCACACTAAACTGTGTAGGCTACATGGGGGTGCAGAGGCTGGCAGGGGCAGGGACCAGCAGCAGCGATAGGAGCCTTCTATCTGCAGAAAGGC
AGGACTGACCCTGAAGGGCCAAGGAAGCTCTGGGGACCTGTGGAGAGCCACCCAGAACTGGGACTGAGAGCTGCACGGGAACCCGCAGACCTGG
CTCCGTGGAGCCGCATTTGTGTTCTGCCTCCTGCTGTGAGAGGTTCGCCACCACAAGCAAGGGCTTATCCTGTCATCCATTTACCCAGAGAGCC
CCAGTGAGGCCGGGATTTGTAGGCCCAATGGTCAGGGCTAGGGGCAACGCCTCAGAGTCAGGGCCTAGTGTGAGGCTTATAAGGGGCAAGGGAG
TGAAGAAATAAAGTGGACTCAGGAAACCAGGGGCTGCCCTTCCCCTCATCAGCCCCCTCTGCCAAGGCTTAGCAGAGTGTCCAGCAGGCTCAAC
CATCTGGGAGGCTGCTGACCACAGGCAGCATGGCATTACCTAGGGCCAGGGTCTAGGGGCTCTGAAGGCCTCAGGGGTCAGGGAGAGCTTCCTG
GAGGAGGTGAGTGCCAGCTGAGCCCCAAAGAATGACCAGACTGTGAACCAAACTGAAAGCATTCCACGCAAAGGGAACAGCTGGGCCAAGACCC
TGTGGCAGCCACACTGGGGCAGATAATGGGGGCTTTGTGGAGGCTAGGACTGCTCAGGGAAAGAAATGAGGCTGGAGGTGCCCCCGGGGGGTTG
GAGACGGCAGAAGGAAAGAAGGTTATATTTTGCTTTTAACTTGCTAAATGCCCCTGAGTAGATCACTGCCCCACTTTTGTGCCTCAGTTTCCCT
TTTATCTCCTCTGAGCTTCAGGCACCTGGGGACCCACACCAGGGAAGTGCCTCAGTGGAAGAGTCCGCTTCTGGAGGCTTTGAGCTGATGCTGG
GGATATGAGCTGAGGGAAGTATGGGCTTCCCCAGCACCTCGGGGGCAGTTGGCCCAGAAGGCTGCTGCTGGACCCAAGGCTTCTCCCCAGTGGA
GGAGAGCTGACCTAAGATTTCTGGGGTTTGCCAGGCTGGACCAACCTGAGAGGATGAGGGATTAGGTCCCTGGGGCAGCCCCCACTACAGACTA
AGGACCTGGGTCCCAGGATAAAATGAGAGATGCGCCTCCCTTCTCTTTGCTGTGACCAACTCCTTCTTTCCTCCTTCTGCCTCCTTCCCTCCTG
CTGGGGACAGGGAGGGGACAATATTTATTCCTTCCCAAGGCTGCAGCTGACTGATACCCTGATGCAGAGGAAATGCCATGCCAGTGCCAGGCAT
GCTGCCGGCGCTCTGGGTCCCTGGTGCCCTCTCCAGGTGTGGCTCCTCCCCATACCCCTGCGCTCGGCCGGTCTGTGTTTGCAGAGCCTCCACT
GACCTGCAGCGGCCTCTCTCTCATGCCTCTCCTATCAGCAGAGCGCCAGCCATGGGCTCCTTGGACTACAAGTCCCCGGGATTGCGCAAATTCC
CCCAGCAGTTTGCACAGCAGCCAGCTCAGGCCCCGTCAGATGGCCCAGGTTCCTGCTCGGGAAGCCCATTCTTTTCTGGAATAATAAAGCAGCC
ACCCCACAGCAGGGGGTGGCAAGCTTCTCTTATCCTCACAACCCCTGACTCATGTCACCCTCATAATATCAAGTGGGCTGGGGCGGGGTGGGGA
GGTGGGTGGCGTTTTCCAGAGGGGGAACTGGAGTCAGAGAAGAGAAGGCATGAGACTTGACCAGGGTCACACGGCCCAGCCAGGACTCCAACCA
GGCCCCTGCTCAGCCTTTTTAGACCGAGCCAGTGTTTCCTGTTCAGGGCCATGCAGGAGGTGAGTCAGGAGCAGGCCCTTTGCAGGAGCTGCTG
GGGGCCAGCAGGAGAGGCCACCAGTCTCTGTCTTCCCTCTTTGGCTCCAGCTGCTTTAATTGGCTGGGGCTGCCAGTGGCGCTGACATTTACCT
GGTGGGAAGGAGGGCCTCCTCCCTCTTGAATTGAGATGCTAAAAATAACAGGGCCCAGCTTCCCAGGGCCACTGCTCAGCCAGCGCCTGCCTGC
CCCCTCAGCTACCTTTTTCCTGGGTGCTGGAAGCTAATTGCTTTGCAAGCATGCAGTTCCTTGCGCTGAGAACTTCCTAGGGATTGGGACAGGA
GGCATTGCATGCTCAGCTTCTGAGTCTAAAGAGGCGTCTCTGCAAGACCTCCCTCCCCTCCAGGCAGCAGGGTGATGGTCTTCTGCCTCAGGTC
TCCCTCCCCCTTTTCTCCCTGGGTGCAGTACCTGTCCAGGTTGACTACTCCACCCCTAGGTGCTTGGGTGCCTTCTTTGAGCTGCCAGAGTTTC
CCCTGCTCTAAGAGAGGGGGCTCTGCCCTCCTTGGAGTTTTCTAGTACTCACCTCTCCCTGCACCATACTCGACCCAGACATATGCGGTGTGGC
CCTCTGCCCTCATGCTGTTCATTTTGTCATACCAGCAACCAAGAGGAGCCCAGGCTTCGGAGCTGGACAAGTTTAAATCCAGCTGTGTGACCAT
AGCACATTTCCAAGCCTCTCTTTTCTCATCTCAGAAATGGGCATGAATCTGACCTTTGCAGTGTTGTTGCAGAATGAATCGCAGTGGGCAAAAC
ATGCCTGATCAAGAGTGGAGCCCCTGGGGCCAGGAGGAGGCGTGTGTACATCATGGGGCGTTCCTTTCTCTTGCTGTGTAAATAGACCCTGGAA
CTCAAATAAATTAGATTGATTGGACCAGAAGGGGAAGGTCTTGAAGACCCAGGCTAAGGATTAGGGACTCAGTCTCCCTGAAAACAGGGTATGC
TTGCCAATCCCCAGGGCACCTCCAGAGTGGCCTGTGTGTTTGTCCAGTTCTTGCCCTGGGTCAGTGTGACCATAATCATTTTGACATTGGCCAG
CATCTCCCGGGCTTTGGGATCATTCCAGTGGAAAAAGTAGTTTTCAGTGCTCTATGCTTGGCAGGTGTGAGAGCCTGGGCGTGCTTAACCCATC
CACACCTCAATTTCCTCATCTGAAAATGGGGATGATAATACTTCCTTCCCCGTTAAGTATTGTAAGGATTAGATTAGCAACATATGCACAGTAC
TTAGAAAGTCTGGTGCAGGGTAACACTTGGTATGTGTTAGCTGGTACTCATAGTAGTAAGAATAGTCTAGCAGTTGGGAGTAATAACAGCAGCC
ACCTGCCGTTTGTTTAATGGTTTTTATCAGATGCTGTGCCGGATGCTAAGCACTTTAAGTACATTATTTAATGTCACTCTCCCCCACCTCCCAG
CAACCATACACAAGGTACTGTTATAAGCTCTGAGTTCCAGATGAGGAAACTGAGACACAGAGAGTAGTTGAACTGAAAACCAGAGTTCAGCATC
AGCCCCACTAGGGTGGCCGTCCAGATTGCCTGGGACCGTCCTGGTTTTAGCATGGAAAATTCTGTGTCCCAGAAACCTGTCACTTGTGGGCAAA
CCAGGATGGTTGACCACCCAAGGCCTGCCTGAGTCCACTGGCCCCACTCTGAAGCCCACAGGATCCTCTTCTTGGCGCAGGGGCTTGCTGGTGC
AGTCGCTCGGCCCCTTCCCACACCTCATAGGCCCCTCCCCCAGCCAGATTGTGCCTGCCCATCCCCCAGGGCCTGGCTCCAGTCTCCTCTCTGG
CTTCCTCTCTTCCCTCCCCTCTGCCAGCAGGAGGGGCATGACTCATGCCTGGTCGGCTCTTTGCTCAAGGCTGTGACTGCAGTTGTCTGGTGGA
CTCTGAGCTGCCCCTCACCAGGCCTCAGTCTCCCTATTTGCACTGAGGGCTTTAGTGGCTTTGCTCCCAGCTTTCTGGAGACGTTCAGGAAACC
TCTTCCTTGAAAGAGGGAGGGCAGAGCGAGCCCCTGCCCCCAGCCCTGGCCTGAGCAGTTGGGAGGGATCGCACTGGCCCTTCCGGTGCATTGA
TGGAGTGCACTGCTGACAAAGGAGAAAGGTCAGCAGGGGGACAGAGCGGAGTTGGTGGTGGCCTGCTGGCCTGCAGTGCCCTCCCACAAGATAG
GGCTTCCTGGGGGAGGGGAGCAGTGCTCCCTGGGCTCTGGGTCAATTCATATTGATGCAGGCAGCTGCTGGTTCAGCAAAGCTGGGCATCCAAG
CCCCCAGAGGGCTGCGGGGAGATGCCTAGGTCATCCTTGACACCTCACTTTCCCTGACCCCCACATCCAACAGTTGATTAGGCCCCCTTAACCA
TCTCTGGATCTGTGACCCTCTCAGCATCTCACCCCTGCTGGGGACCATCTTCCTGCTTCTGAATCCCTGAGCCCTTTCCAGCCCTCCCTGGCTT
CCCCCATCACCTTGCATAAAGTCAGAATTTTGAGGCTGACATTAAGGCCCTTACTGAGCTCAGAAAGGCTGCCCAGAGCTGCCCAGGCCTTCCC
TAATATCAGCCTTCTTGTGCCCTCCACCCTCCAACCCCAAGGTCATGTGAAAGGTCTGGGGACACCCTGAGTGCCAGGCCCAGCGCTGTCCAGT
CAACACTCCAACCCTACCCAGTATACAGGGCTGAGCCTGCAGCAGAGCTGCAGCCAGGTGCCAAATGGATGCAGTGCCCCTGCCGTGCCCTTCT
CAGACTCCCCCTTGAGCTGCTCCCATGCGGTTGCCCAGTGGATCTCATGCCCCAGCTCCCAGAGGGCAGGAGTTATCTGTCTCCCCAGCACACA
TGCAGACCAAGATGATGGCAGGAAAGCGCCATGGTTGGTGCATAGGCGCTGGGGTCATAATATGAGGGTCTGATTCCGGCTCCATCACTGACTA
ACCGTGTGACCTTGGGCAGTCACTTAACCTCTCGGTGCTTCAATGACATCATTTCTGAAATGTGGGTAATAATATTCTGTCCTTCACAGGATAG
TGGGATTCAATAAAGTGACACTTATGAAGTGTTTGCAGGGTCCCAGAACATTTTAAGGATCAGTAAAGTGAGCTCCCATCAGTCATCATCAGGC
AGGTATCTATGAAATGAGCACCGTCTTCCAAATGGGGCCCTCCCAGTTCTTTGAAGGGTCCTTGCATTTTTGCCCGGATTGTTCCCTCTTTGTC
TTCCTCCTGCTCTAGCATCCCGCTGCACCCCCGCCTGACATCTCCTGAGACTCCAGGAAGCCAGCCTGCCCTAGCCTTGCATTCTTGCCTAATC
CACCCCATTGCCATGGAATGCCTTCAGGCTATGGCCAGGAGTTCTTCCCATGTCCCCCACAGCTCTGCACAGGTTCATGGACCACAGTGGATGC
CTAATAAGAATTTGTTGGGTTGAACCGATATTCACCAAGCAAAGTCACCTCCAAGAACAATGCAGCAATGACCAGCCAGGCCTCTGGGCCCATC
CACAGCCCATTGAGGACATTTCTGAGACCAGTCCTGAGCTACTCTGTGCAGGAGAACAGCACGTGCCCACTCTGGCCTCTTCGGTGGCGGGGGA
TTGGGGATAGGTGGGGGTGGCACTGGGGCTCAGCAGCCACACTGGAAGGTTTTTCAGGGGGTTTATCTCCCTCTTCTCTGAGCTGTCGTGCCTG
TTTTGTTTGCCTCGGTCCCTTCCCCACCCCCACAGTTCTCTGTTAAATTGTGTTATTATATTTAAACAAATCAATTCCATTGCAGAGTTTTTCC
TGTCCTAATGATGAGTTTGCAGCACAAATGCCATTGATTTCTCACAGCCCTGAAAACCAGTTGATGAATAGCACCAGGCTTGTCTTTGGAGAGG
CACTTAGAGCAGCTCTGCATGGCCCCCTCTGCTTCTCCCCTGTGCTGTCCCCCTCATTCAGGACCCCACCTCCACCCCACACTCTGCTGCCCCC
TGAGTTGGGGCTGAAGTCATGTGATAAAGGGAGACCCTCTGTGCTGGCCAGGGCTGGAATGTCATCGCAAGGGCCCGGCCCCTGTTCTGGTGGA
ACAATCATAGTGGAGATTTGTATTGATTTTGGCCAAAGGCACAACTTACAGAGCAGAGAAGTGGTGCGGTGTGATCTTTGTCTTCCCTTACAAG
TTCAGAGCGCGTTCCTGAGAGTGATGGCAGATAGCACCTCAGACACCATTTCTAGACCTTGCCAGGGCTGCTGAAAGACCTTTGAGCACTGCTG
TTTTAAAGGGAGGCTCCAGAGGTTTGAGAGCTGTCTGCACTGGGGAGTGTAAAACTTCCCTTTGTGAAATCCTTCAGAACCTGCAGTGTTCAAA
GTCACGGTTAAACCACTACCCTGGGCTTTGACTTTCAGATCACTGCCACGTGCACGAGCTTCTCTGAGCTTTCTAATGACCCCGGGGCTTGGGT
GGGCAGGTAATATTGTTCCCCTTTAGAGGCCGGCAGACTGAGTCAGGGTAGTCACTGGAGAGAGGGAAGGGCTGCTTGTCCACTCCAAGTCCTG
AGTGCAGCAGTGGCTCCCATGTCTCCTCCTACAACGGTAATACATGGCAGGCGAGCTACTCTGATCACCAGCCTTTTCGGCCGGGCTTTCTGTC
TGTCTGAGCCCAGGTCCTCGTGCAGGTGTCTCCACTCAGCTGCTGCCTGCAGCATACCACACAAGCCTGTGTATTGGGCTGGAGTGAGGCAGCA
GGGAGCAGCGAGGACTGTGGCATTCTGGAGAGTGCCTGCCCCACCTAAATTCCTAAAAGTTTGGACAACCCATGCCCGTAAAAGTATACTACAT
GGGATGAGACTGGGGGCCAAATTTGCTGCCTCTCCCCTATTCTAATTCAAGCCTTTGTTTTGGAGACTAGAATGTGTGGCCCAGAATTGGAAAT
GGCTCTTCAAGGTCACAGCACAAAGCAGTGGTGAGCTGGGGTTTGGGTCTGGGAGAAGGCGATGAGGCCCCACAGCCAGTGTTCTCTCCCCTCC
CTCAGGCTTGGCGAGGCAGTGGGGTGGGGGCAGGGCCAGGGACTGTGTTCTAGGGAAGATGGGCCCCTCACCCCTGAGTTCAGGGGTAGCTCAG
CAGGCCCCACTTCCCTTTTTTCTCCCTTCCTCCTTCCCTCCCATGCAGACAGACCTCCTGTCCCTGTAATCAGGTTAGCCTCCTGCTCCGATGA
GCTGGACCAGCTGCCTCTTAATGACCTGTCGACCTGGCCCCCTCGCAGGCTCTGGGGTGGGGGAGGGGAAGCTAAGAAAAGAACGGTCGGTGGA
AAGAGGCTGTCCACTCCCTTGATTCAGACCTTTCCACAGCTTCTAGGATCAAGTCCAAACCCTTAGAATAGCATCCAAGACCCTGCTTGCTTCT
CCACCACCCACCCCCTAGTACACTCTTTTCCTGCAGTCACATGATGCTGTTTCCTACCTCCCTGCCTTTTCACATGCAGGTCCTTTGGCTACTT
CCCACCTTCTCAGAAAACTCCTGTGCATCCTTCAAAACCCAAWTTGTCCCTCTTCCTCAGGACTGCTTCCTCTGGGTTCCTGCCTCTGTACTCT
CACAGCATTGGGTGCTCTGCTGGTCTGGAACTTATTCCCACTGTGTTGGCCTCCATTATTCCTCGATCTGGTTTCTCCGGAGTTCCGGGAGGAA
AGGAAGAATATTCCCTTCCCTCTGCACCTCAGGGGTACAGCACAGAGAAGTGCTCAGTGAAAGTATAGGGCCAGCAAAGAAGAGAGAAGTAGGG
ACCAGGGAAGAGAAAGAGACAGACAAGAGTATAAAAACAGCTCCTCCCAGCCCCTCATACTGCCCTAGCCTGCTTCCTGCCAGGGAGGCCATTC
CTAGCGACGCATGCCTGGCCTGAAATAGACACATGGGGACCTTGGTAGTGCATTTTCATAGCTCAACACTAAGCACCCCCCTCCCCCTTATTCC
TTGGGCACTGAAGCTCCCTCTCCTCCCAGGGCCTTGCCTCTGCAGCCAAGACCCCTCCTCCAGCAGTGCCCAGACCTCACCCCTCTGTTTCTGT
TAACTTTGTTTTATAAATAAATCATATAAATAGGCTCCTCTGATTCCTCGGAGCATGCAGTGTTTAAGGACTGGGGCATTGCCGCAGGCATAAT
TGTACTACCGAATCCATCAGCTGCCTAATTACCATTCATTTTGCATTTATTTCTGCTAATTAAAAAGAGAGAAACTCCTAACAACTTTTACATA
AATACTATATTTGTTCTATGTAAAGATGGATGCTAATTTGCTGGTGATTAGCTGATTATCGGATTTTATAGCTGTCACACAGGATGGCCCTCTC
TCCCATTTGTACCCCGGACACCACTTGAGGCCCCTGATCAGATATGGGAGGAAGGGGTCAAGGGAGGCCCACCTGGGGGGTTCGAGGGCCTGTC
TGAAGCCCGGGGTCACCAGGAGCACCAGATGCCACTGGGGCTCAGGGCGGGGTAGGGGGAAGGTGCTGCAGTCTGATTGGTGGAGCTGGGAGGA
ACCTGTGGCTCACTCACCGCTTCTGTATAGATGGAGAAACTGAGGCCCAGAGAGGCCTAGCACTTCCCAGCAAAACAGTGACCAGGGACAGAGT
CAGACTGCTATTCTAGACTGGACTCCAGTGCCCATCCCACTGTTGTATGCTGTTGTCAGGAGGAATTTGCAGGGAGGTGGCTTTATCTGCTGCT
GGAGAACTATGAGGACCAGACTCCAACTCATGCAGAGGAAAGGAGCACTTAGCCCATGGCCCAAACTGAACCCTCTGTGTACCCTTCTGAAAAC
CTCCACCTCCTTCTACTTCCCTGGGCAGCCCTGGGCTTTGCCCCACGGCACCCTGACTGCACTTAGCTAGGCTTTCTCCCAACCTGGATTGAGA
CTTCCCCAGGACTGGCCTTGCACCCTGTGGGCTATCCGGACACATGTGAGGGTGTCAAATTTTAACACTATGTATGTATGTTCATGTAACTAAG
GAAAAAAATATAATTAATACATCAAACCCATGATTTCACAGATCATAGTGCTTTTGATGAGGCTAAGTAAAAAAAAAAGTAATTGGTTTAAAGT
GATATAAATAATTAAATAGTTTTTTTTTTTGAGATGGAGTTTCGCTCTTGTTGCCCAGGCTGGTGCTATCTTGGCTCACTGCAACCTCTGCCTC
CCGGGTTCAAGTGATTCTCCTGTCTCAGCCTCCCGAGTAGCTGGGATTACAGGCATGCGCCACCACACCCAGCTAATTTTGTATTTTTAATAGA
GAAGTGGTTTCACCAGGTTGGTCAGGCTGCTCTCGAACTCCTCACCTCAGGTGATCTGCCCGCCTCCGCCTCCAAAGTGCTAGGATTACAGGCA
TGATCCACTGCACCTGGTTGATACATTTTTAAGTACATAAAGAATTAAGATGGAATATGGACACGGCTTACAGTACCTAATAAGGTGGGTTATT
TGATAACTTGAGGAGGGTCTGAGAAGACCAGGTCAGGAGGCCAGGCAGTTGAGGTCAGGGTCAGCAGTGACCTGGGGCAAGGTGGGCCAGGTCT
GCAGCCACCACTGCCCCATCCCATGACTGGGCAGATGGTGCCCTCAGATTGTGCCCTTCTCTCCCCACCCCTGCAGTTGGTCAGAATTAGCCCA
CAAGAGAGCAGTCATTTGTCCTCCTAAAGGTAGTTTCACTGGGACTCTGTTGGGCGAGGCCAGGAGGTTGGACATPACCCCATTTCCCACCAAG
CCACCAGTTGCGAGTGTGTCACTTCACATGGACATGCGGTGCACCTGTTTCCCCTGGCATTGATAGGTGAGACACTGACTGGCCCCTGTTCTCT
TTCTTCCTGGCCAATCTGAAAATGCCCAGTTCTCGAGTTAAAGGGATAACAGCTTTGACAAGGCCAGGTGTTGTCTGGGTGGCCAGAGAGCTAA
GGGTCAGGGTCAGCGTGGCCCATTGGCCCATTGGAGATCCCCAAGGCTTTGGGGTCCAAGGGCCAGGGTTCCAGGCCTGGCTGCTGCTTCCCAA
AGCGTCAGTCTCCTCCTCTATAAGATGGGGACAGTTGTGCTGAAAGGGTTCTGAGGGTCATGTGCAAAGTGTCCCTCAAAGGAAGACTCAGTAC
AGCCGGCAGTGAGCCCAGTTCCAGGGGCCCTCTCGGCCACCCACCCACCCGCCGCCGCTGTGTCTGTGCCGCCAGAGATCTCCCTCTCATCAGC
CCCTTTATCGCCCTGTACACAATCAGGGGAAGCTGGGAAGGCCGCTGGGGCTGAGGGCAGATGGAAAACAAAGAGAGAAAACAAAGGCACTGCT
CCTAGGGGGCCAAAAGGCACCTGCCTCCTCCCCCAGGCCTGGCCTTGGCCACCCCAGTCCTCCAGGATCTGCCCCACCCCTCCAGAAGGATAGT
CTCAGAACTCATGTAGGTCACCACTCTCAGGGGACCCTCAGGAAGTCACTCGTCTCCAGGCCACCTTTTCTTATCTGTAAAATGAGAGAATTGG
ACAACAGGCCATGTGACTTTTGGCAAACCTTCCCCCTCTCCCCACCCTCCAGGGTCTCCTTGGGGCAGGCCCAGCTCGTCTCTAGGACCCCCCA
GCCTGTCCCAGGAAGTCAAGATGGTGCCCACPTCTGTCTGTGCCAAGGACCCCAGCCCTGCCTTGGGCAGGGTGAGGTAAACAGGAGGCCCCTG
TTTCCTGCCGAGATCTTGGGGTGTGCTGGGTGCTCAGTGCATCTGTGTGGAATGAATGAATTATTTCATCAGAAGGGGGCCTACTCAGAGTCAG
CCCTAGGTTGCTGCTGCTGTTGAAGCCTCTCAGGGAAGGAGACCCTTCTGGTCACTTGAGGGAGGTGGAGAGAAGTGGTGAAGGGCTGGCAATG
GCATCCCTGCCAGGCTGTCTGGGTTCACATCTTGGTTGTGTGACCTTGGACTGGTTTCTTAACCTTCTGTTTCCTCATCTTTAAAAATTGGTTA
TAGTACTGACCTCATAGGTTGTGGTAAGATTAAGTGGATTAATTTACATAAAGTGCTTAGAACAGTACAGCTAAAGGTTAAATATCACTATCCA
TTTCATCACTCTTATTATGATTAGAGGGAGGCTTACACGGAAGCACATGAAGATTAGGCATCCTGGCCTCTCCTGCGCACAGCCCTGGTGAAGG
CTGGGAATTGCTGGGAGCTATAGAGTGTTCTAGGTGGGAGGGGCAGCAGTTTGCAAACAGGAACATTTCTATGTAAGTATTTCTGGTAAACTGT
TAAACAGGGGCCTCATGAGAAAGGGGCATGGATTCCTAAGAATATAGGAATTTGTTGTGATTTCTTTTCTGATTCTAAATATATATTTGTAATG
TTAAATAATTTTTAAAACAGGGCCTCCAAAATTGTATAAGCCTCAGGCCCTGCAACCTGAGCTGTTCCTGATTGTTTCTGTTATTGTGATTGTA
TTTCTGTGGCCCACCCCTGTGCTGCCCACTTTGGTGCTCCTCACAGGATGGCCCAGCCTTGGGGTCCCAGGGACCTGTCTTCTCACCCTATGGA
CAGCGGTGTTATGTGCAGGCTGGCCTCTGAGCTGTAGTCTACACTTCACACCCCAGATGGAGGGAGCTCTGAGGGAGCAGACAAAGGAAAATGG
GTACCTCAGGGAAGGTCTCTGAAGCAGTCTTCTCTGCATATGCAGGACACAGAGCCCTGTGCCCAGAGGCTTAGTGGCCAGCCCTGCTGCTCCT
ACTCACCCGGACTAGGTGTGGCAGGCCAGGCCCCTCTCCCTTCCCACTCCCCCTCGGTATGTGCCCTGGTGGACAGGCAGGGGCGGGTGGGGCC
GTGGGAAGCAGGCTGCTGAGTTGGAACAGTCACCCCTACAGTGGTCGTTGAGTGGCTGGCTGTGCACCAAGCTTCAGGTTCCTGCTGGGTGCAG
ACCCAGAATGGTCCTTGCCCTCTGGGAGCTCTCCCGGTAACTGGGACCACAAGATTTGCACCTGGAAGGGCTAGGGGTGAGCAGGGGAAGGCTG
TACTAGACCCATCTCTCTCCGAGAGGAGACAGGAGAGCCCAGAGGTCGTGAGGGCTGGACCCTGGGGGAGGACCTCTGAGAGGACGGGCCCTCG
GCTGGGGGGGGGGTGCTCCTAGGGTCGGCGCTGTGGGCCCTGGCGCCTCTGCCAGCAGCAGGGCCTCTCGGCCCGGGCTCTGACAGGGACATTT
ATAACTCACAGCTGTGCGGTCCTGGGCCCAACTGACTGTGGTAAACCGATCTGGCTTCAGGAAGTCCCGTCCCGAGGCCACTCCCCATCCCGAC
CCCCACACTCTCCTTCACCTCCTGACACCCACATCCTGTTCTCAGCGCGAGGGGCAGGCGGCGGGCACTGGGCCAGGGGCCCACCCAGGGTCTC
CAACCTGTGGGCAGAGTGGGAAAGGACAGAGAGCAGCAGTGAGGCCCCAGTGCTGCGGGGTGCCCACCATTGCCAGCCTCCTCTCAAAGCACAC
ACAGGGAGGCAGAGGCCCAGAGGCAGTGCAAGGCCCCCAGGAGCCCACAGGGTCAGAGCGCCAAGAGCATAGGTTGTAGGGCTCAGGGGAATGT
TCTGCGCCTCCAGATCTACCTAGAAAGGGGATTCGCCTTCTGGCCTCTGTGGACCCACTCCAGCCAAAAGGAGCCTGACCGGAGGGGCAGAGTG
AACCAGAGGCCTCAGAGAATGCTGGGAAAGACGCCATTTTGGGCCCTGGGCCTTGGGCTCGCCCTCCTTTGGGACACCCCGTCCCCAAGATGCT
TCGTGCCCATCCCACCTAGATCCTCCCAGCCTAGTTCAAGTGTGGGGCCAAAAATGGTCTTGCCCAGTGCTAGAAAGAGAGACAGTACTAAAGG
CTGCAGCACACCATGGCTCTGACCCCTTACATCCCTTGGCCTGATACAATGACCAGTTCTTGGGCCCCATCAGCCAGCCCGCCCCTCCTCCATG
CCTCTCCCTCTTCTTCTGCACAACGTTTGCCAAAGCTTGGCCCAGGTAAATCTGGTAGTCTACAGCAAGATTCTAGAATTAGATAGTACCCAAC
AGGACATACATTTTTGTTTTACTAGTTACAATTTATTAAAGAGTATCAGAAATATATAACTAGCCTATCAAATCAATTATTTCAAGGGCATGAT
TGCTTTGGACAAGGCCAGATTTATTGGAGCTTAAAAAAAAAGTGAGTGGAAGGTGAACATTAAATTAAGTCATCACACAGGTAGATCTCAGCTC
AGACCAAGAATCCTAAAGAGCGTGGAGGAAGACCTGAAGTTTGGGGAGCACAATCTAACTCATCACCAGGACTCTTTTCTGCTTTAAACTCTGC
ATCTGGGCCTCCTCTCCCAGTTATAAGCAAAACAGCCAGCCCTGAACCTTGTAGGAGGGGCAAGGGAGTGAGCTGGTTTTCCCGAGGTGCGTAC
ACACATAAAATGGCTCCGAGTGTTGGTCAGGGATCTGGTGAGCAATTCATAATGAGGAAAGAATTCAGGCAGCGGTCACACAGTGGCTTTGGAA
GTGGCTGCCGGCCGGCCGTGTGAGATAATTCCCCGAAGCCAGGCACGGCGCAGAGAGGAGGCCACGGCAAGGGCTTTGTACACCCGGCTAAAAA
TACCCCCGACAGCTTCTCCCTGTCACCCTGCCTGGGGGCCGTACAGGAAAGTGATGCTGTTCTGCTTTTCCTACTAAGAAGAGAAAGACAACAC
AAAGTCCCCAGTCCTGGAAGAAACAAAACTTATATTTATATATTTTTCAAAATCCCACTAGGAGGAATTGGCTTGCTGGTTGGAGTGTGGCTGG
GCGGGGAAGACACAGCCAGCTCTGATAAATCAGGCATCCCGGGGTGTGCTTAAGTGACCCAAGAGGCCGCTGAGATTAGCTTGGCGGCAGGGAC
AGAGGTGGGAGGGGAGTGGACGGCACACCGGCCTGCTCTCACACACGTCTCATCTCTCCCCTGTGTACCCAGGAGGAAGAGGAGGAGGCCCGTT
GGCGTCGGACCAATGCTGCAAGGGGTGTGAGGAGAGGAGCCGCTCTTTTTCACTGAGCTGCCATACCCCGAAACGTAACACCAGCCCCCGATGC
CCAGGCCATACCATCACCTTTGCCTAAGCCTGGGCAGGGGCAAGCCCTAGCAGCCCAGAAGTGCTGTGGCTGGACCAGAGCTGAGGGTTTTCAG
CTGCTTCTCACAGCCTTTCCTGGGCCTGGCTGCAGGGCCTCTCTATAACTCCTTGTGTGGTGCTCATACTGCCAGAAAAGGCTTCTGGGTGTCA
GTTTGTCTGTCTCCAAAATGGGGCACTGGGCTCAGGTCCTGTACTTACCTCAGTTCTCAACGATTCTGTGAGTCAGTTGTGTCTGATTTGGTTG
AGCCATCTTAGCAGCCTGGCTCAGTTGGAACTCTAGGGCTGTAGCCAGGGGTCCCAGCCTTTGTGGATGCTGGATTCTAAGGCTACGAGGTCCA
CAGTGCCATGTGGGGCTTGAGAAGGGAGGATTTTCCAAAGCAGACCTGGCTGGAGATGTGCTTCACTGGGGATGTGGCTGTGGTCTCCTTTGTG
GCAGCATTCAAAATTCCAAAGTTCTCCCTGTCAGCTCTGGGCAAGAATTATTATCTGCATTTTCAGACCACGAGAACAAGGCACAGGGAAGTTA
ATGGTGCCTGCCAGGGATCACACACCTAGCTCTGGCCACGCCACAGCAGCTTGGCTTCATCCTATTTGCCATACCAGCTTCTATGCTTTTACTT
CGACCGTACTGCACTCATCCTCTCAAGAGAAGTTCTGCTTTTGAGGCCAAGCAACCACTAGTCTGTATTGTGTGCCTGTGAGACCCATCCCTCC
ATCCATCCATCATCCATCCACTTACCTGTCCATCTCICCATCCATCCCTCTTTCTTTTAGTCCTTCCACACTCATCCACCCATTCATCCCTCCA
CCACCTATCCGTCCTTCCATCCAACTACTTACCCACCCATTCCCCCCATCCACCCATCTATTTACTCTTTAATTCACTCATCAAATATTCATGG
ACTCTCACACCAGGCCAGGCCCGCATTAAGGGCTGGGGTTTTTGTTTTTGTTTTTGTTTTTTTGAGGCAGGGTCTCATTCTGTCACCCAGGTTG
GAGTGCAGTGACACGAACATGATTCACTGCAGCCTCGACCTCCCAGGCTGAAGCCATTCTCCCACCTCAGCCTCCCGTGTAGCTGGGACTACAG
GCATGCACCGCCACGCCCAGCTGATTTTTAAATTTTTGTGGAGACGGGGTCTCACTTCGTTGTCCGGGGGTGGGTTTTGAAGACAGAGCCAATT
TAGCCCTGCCCTGAGAGGGTGTGGTGTGCAATGGAGTACGACTCAGATCCCAGAGCCAGACTGAGTTCCATAAGGCAGTGCTCAAAGCCTGTGT
GGTGACCATGACGACACAGGGGACGAGGGAGAGGGGACGTGGCTGATTCTGCAGGCAGACATTCGGGGAGGAGGAGGCGGCATTGCTCATGCAT
TCATTCCCCAGGTGTTGACCGAGAAAGTCTGTCAGGAACTCTTTAAGTTCAGAGATATAGCAGGGAATAAAGCAGACAAAACTCCCTGCCCTTA
CGGCGCTTGTACTCTCGTGAGGAGGCATGGATGATAAATACCTAAAAAGTGAATAGGTCAGGTGACGCTAAGTTTTAAGAAGACAAATTTAGCC
AGGTAGGAGCACAGAGTCTACAGTGTAAAGTACTCTTTCACTAGCCAAGGAAAGCTTTTTTTTTTGAGACAGGGTCTCACTCTGTCACTGTAAC
CTCGACCTCCCCAGCTGAAGTGATTCTCCCACCTCAGCCCCCTGAGTAGCTGGGACTACAGGCACACACCATCATGCCTGGCTAATTAGTGTAC
TTTTAGTAGAGATGGGGTTTTCCCATGTTGCCCAGGCTGGTCTCGAACTCCTGGACTTAAGTGATCCACCTGCCTCAGCCTCCCAAAGTTCTGG
GATTACAGGTGTGAGCCACTCTGCCTGACCCAAGAAAGGCTTCTGTTAAGTTGGCCATTTAAGATAGCTGAATGCAGCCAGGGAGTCTGCAGGG
ATGTACTTGACCTGTTCAAAGAAATGCAAATAATCCAGTGTGGCTGGGAGGATGTCAGGATAGGGGGCGAAGTCAGAGAGATCATGGAGGACCC
ACTCCTGTAGGCGGACAAGTGTGGAGCAGAGAGATAGCTGGGAAGCTGTTGCCCACATCCAGGTAGAGGTGATGGGAACAGGGACGAAGGGGTA
GTGGAGGAAGAGGTGGGAAGCCATCAAAGGCTGAATATATTTCAGAGACCCAGCAGGCAGGCTCGCTAGTGAATTAGAGGTGGAGGATTTAAAA
AGACGGAGACAGTCAGGGAAGGCCCTTGGGTTTGGGACAGGAGCCAGTGGTAGAAGGGAGGTGGTTATTTGCTCACCCAGGGACAATGGCCGGG
GAGGGCTGCGTGAAGCTGAGGAGTTCTGTTTCAGACACATTGAGTTTGAGATGCCTTTGAGACGTTCAAGTGGAAAGATTGAGGGGGCAGCTGG
AAATATGAGTCGGGAAGGCAGAGAGAGGTCCCAGACTAGAGATATAAATTTGGGAGTTGTCGTGGATTGATGGTGTTGCAAGTCGTGAGGCAGA
TGAGATCACTCGAGAGTGTGTGGCGCTTGAGAGGGTCAGGAGCCTGAATGTGAGTCACGGGGCACCCACCCGTTTAGCGGAGTGACTGGATAAG
GGGTCTGCAGAGGAGCTGGAGAAGGTGCAGCCAGGGAGGGCAACAGAAAGCAGGCTCTGAGTGGACAAGCAAGACTCTGCTGGGCAGTGCATGG
AAAGGACTCTGTATGCTGCAGTGGGAACAGCCTACACAAAGGCTCAGAGGCCTCAATAAACCAGCCATATTTGGAGAGGACTAATGGAGAGACA
GGAGAGACCACACCGCCTTCCTGCCCACACTTGGCCACACAGGGGCTAGGGGCTGTGGGCAAGCGCATGCCTGGGCCCTCCCCGTCACCCTGGC
CGGCACTCCCTGACCCTGGCCAAGCATCTTGCCCTGATGTCTGTCCACTTTCCCACTTTCTTCAGCTAGCACTATGGACCTCCCTCCTGCTTTC
TGGGGCTCCTCCCCCTTGTCCAAAAACGCAGGGCTCCAGGTGGCAGGTCACATGCTGCCCATGGGGAAGAGGCACCGAGCAGCTCCCTATTGAG
CACCTGAGCATGGTGTGGATCCTTTATCCTAAAAGATGTGAACCTTCCCAGGCCGCGAGTGAGTGTCAGAGCACCTGCTCCCAGTTAGGGGAGC
TCTCTGCCTTTGGTGGGCAATCATATACGTGCGTGTCAGTGTTGTGTCACTTCATTCTTTCTCCATCTCTGTCTCTCTCTTTCTCAGCCTCTAC
TGCAAACATCAGCAGTGCCCCCTCCAGGCCAGGCCCCGGGCTGGGTAGGATCCCGAGGAACAAGTGTGCTCCTGACCTTCCAGGAGCTCCCCAC
CGCTCCATCTCTCCCTTGGCTCCTGAGTGTACCTTTTTGGTGCTCAGGAGCCTGAGCCTGACTTAGAATTCCAGCTTTGCCACCCACCAGTAGT
ATGTCTTGGGGTGCTGCAGCTGCCCTGTACTGCAGTTCACAGGAGAGGGCTCCCCGAGACTCTGGAAACAGACCCAGGAGCTTCTGAGCTATCC
CTCTGCTCTGGCTGAGGTTTGACCCCGGGCCCACTCACCCCTCTCTCCACAGCAGAGTTGTGGGTCTTGTCAGATGCTCTTCCCCTTCAGCGTG
CAGCTCTAGTCCCACCTTCTCCAGAAAGCCTCCCTTGACCACTCTGAGGTCAACCAGTGTTTCCTAAGCACCTGTGTTGTGCCAAATGCAGGGC
TCGAGTCAGGGTGTGCAGAGATGACAGTGGAGGGTTTCTGCGCTGAGAACTGGCAGTCCACATGGGGTGGTGGGGGGACAGGTAAAGAACCCAC
ACCTTCCAGCATAACAGTGAGCACTGCAGGTGCCCAGCAGAGAGAACGGCCTTGACTTGGTCAGGAACGGCTGCACACAGTATAGCTGCCCTGA
ACAAGGCCAGAGGCCGGGCAGGTGTGCAAGGTGTGATAGGGGCTGGGGGGTGGGGGTGGAGAGAAGAGTGTGTCCAGGCGAAGGAGACTCGGGA
GCACAGTCCAGCCCGTCTGCAGAGTGAGTGCGGTCTCACATGGCCAGGACGTGAGAGGAGGCTCAGGAGACTTTTCCAGGCTGAGAATTGAATG
GAGCCAGGCAGGGACTTGTGGACCTCCTCTCCCTCGGGAAAGTCAGGGGGAGATGCCCCTCCAACTCTGCATCGGGGACTAGGGCAGCAGCAGC
AGCTGCTCTGATGATGCATGGCTCCGTGGCAGAGCGGTGGCAGGAGGCTTCGGGTAATCTCATTAGTGTGCCCGGCGGAGGGGACGAGTAATCC
CGTGCCTTTAGACTCACTTTTCTTTAAGCCTGTCCCTATCCATTCTCTCCCTAAATCCTTGTACTGCTTTGGTGACATCAGGCGGGACTGGACT
GGACTTACCTCCATTGAACTGATGGGTAAAACAGGGTTAGAGAGGAAAATTCTTGCATGATGCCATAGCTCACAAGGGGCAGGAACTGCATCCG
TCTGACTCCAGAACCCATGCTCTTATAATCCTCTACTATCTCCCAGACTGTCTTAGACTATCCAGGCTTATAGACTGCCTCCCCACTGGCCCCA
TTTTGCAGGTGAGGAAACCGAGGCCCAGAGAAGGTAAGACACTTGCCTAGGGCCACGCAGCAAGTCAAGGACAGCACTGGCACCAGAAGCTCAC
CTGGCCTGTTTGTGTCCCCACCCCACCCCACAAGCAGGCACAGGGATCCCCCTGGTCCCTGCCCATTTCTGCCTGGAGCACCCACTGTGCTTGG
TCTTCCTGCAGTCTTTGGCACCGGCTCCGGTGGAAGGCACCAAGTTCCTCATCAGAAGGCTCCATTGTGCCTATTGTTTAAATATAGATTACTA
ATGAAATGCCTTTTCATTGCCTTCCCAGTGAATAGTTTCCGTGTAAAGTTAATTTGCAGTGTTATGTAAATTCTGTTTAACTTCAATCAAGTTT
CTAATTATGTTTTTACCGCAGTAATTCCCATTTGATTTGTCATCTCCTGTTAGGTATTCAATGTTCGGTGGCCGGGCTTGCTTTCCGTCTGCCT
CTCAGTTTTCCAAGGCCCTGACCTCCTCTCTGCTGTGTGTCTGTCTCCGTGGCTCCTGGCTTCTTCATCCCCTTCGGCTTCTTGGTGGCTCATT
TCCCACCTCCTCGCCATCCCTCCATCTATCTCCTGCCCTCCACCTTCCTTCTTTCTTCCTCTTGCCCACCTGCACCTTCTTCCCCTTCTGTTTT
GCTGCGTGCTGTGCTGGCTCCTCCCCCCTGTCTGCCTCATCTCGCCCGCCGCCATCTGGGCCCCCTGGGCTGCTCCACCAGGTGATGGTGCTCT
GCTCTGTTTATAAGAGTTCAAAACGTAGGATGGAGTAGTCAGGGGACCGGGCTCTAGGGCCCAGGCTCCCCCAACACCAGAGGTGACCTTGGGC
AGGTCCCCTTCCTCTTCTCTGAGCCAATTTCCCCCATTGAAGAGTGGATATTAGGCTGGCCAAGGGATTTCAGCCATGGCCCCAGATGAGATCC
GAGGCAGCTTTGAAAGATAGCCCAGCCCAGTACAGTGGCTCACACCTGTAATCCCAGCACTTTGGGAGGCTGAGGCAAGAGGAGCACTTGAGCT
CAGGAGTTTGAGACCAGCCTGGGCAACATGGCAAAACCCTGTCTCTACAAAAAATATAAAAAATTAGCTGGGTGTGGTGGTGGGCGCTTGCAGT
CCCAGCTACTTGGAGGGTTGAGGTGGCAGGATCGCTTGAGCCCACGAGGTCGAAGCTACAGTGATCAGAGATTACACCACTGCACTCCAGCCTG
AGTTACAAAGTGAGACTCTGTCTCAAAAAAAGAAAAGAAAGAAAAAAGATAACCCACCCCAAACAAGTTAAATCAGATCTCCGGGGCGGGGCCT
GCGGCCAGGCTTGAAAGCTCTAGTCTGGATGGCCTCAGACGGGCTGCTTTCTCCGCCCTGCCCGGATCCCCAGGCAGCACTGTGGGCTGGTGCA
TGGGTTCTGGAGCCCAGAGGCTGTGCTGAGTACAGCTCTACCACTTTCTAATACTGGGCAAGTGAATTCATGCCTCTGAGCCTCTGTTTCCTCA
TCTGTGAGATGGGGCTAACACCACCCACCTTATAGAATCTGTACCAGTGTCAGCCACCATGTTCTTTGATCTAAGCCAGTTATGCACCACTGCT
GCCTTCACTGCCCATTCATTCTTCCAGAAAATATTCGGGGATTCCTGGACCTGCCAGCCTGGTACTGGGTGCTGTGGCAACAGGAAGAGGAAAC
CCACCATGGGGGTCCAGCCTTTTAGGCCTTTCCAGGGCCCCAGAGGATGGGTTCTGTCTGTTGATGGGCAGGTGAAGGGTGACTCCCCTGAGAA
TCTCACCGTGTCGCTGGACTGAGGGAACCCCTCTTTCTGAAAGAACGAATGAGACCCCAGATTCAAGGGAGAAGAAGGGGCATCCTAGGACTTC
CCTGACAGCCTCAGCTGGGAAGCCCCTGGGGAGGCTCTGAATGCCTTCTGGGGTCGGCCCTGGCCCATGCTCTGGGCAGATGCTGGCTCAAACA
GGGCTGGCAGTAGACAGAACAGAATAAGGACCCTCCTCCTTGTAGTCCATCACTGGGCTGGACTATGTCCTCCATCCCGTGGTCCTCCTGGCAG
CCCAGGGCATTGGCAGGCATCGTTCCTCCTGCTTGGCAGATAGAGAGACAGAGATGTCAAGGGATGGGAGGCACTTGCCTAGGTCAAGGGCTGG
ATTTGAACCTCACTTCTGTATAAACAAAATCCCAGTGGGGAAAGTCGAGCTTCCCTGGGAATCTGCCCTGGTGGGACTAAAACGTGATCGGCAA
ACCTGTCATCCCCTTTCTTCCCTGGCCTTCCAGCAGGTCCCCTGCAGACCACCACTGGCTGTCCTTGCGGGAGAGACCTGATCTTCCTGTAAAT
AGCAAGTGCTCCTGTTGCCTGGGCTCTGAAAGCCAGAGGTTGAACCATGCACTTGTCACCCACTAAACCTTCTCCCTCCTCATGACCCATTGGA
GAAACTGAGGCAGAGAGAGGTGCAGGGACTTGGCTAAGGCCATGTGGCTTGTGAGCCGGCCAGGATTTCATCCAGGCCTGGGTGAGGTTAGAGC
CTGTGCCCAGACCCGCCACCACCCGCTAGCAAGAACAGGACTTTCCTTTGTCCGGCATTTTTAACTCTGGGTCTTGGCCATCACTTCAGACTGG
CCTGGCCCACGTGTGGCCAGAATCCCAAAGGGATGCCTGTTTGGGAAGCGTGACCGCCAGGGGGCTGGCACCTGGCCCAGGAAGGGGGTCATCG
AGAACACTCAGTGCTCAGTGGTTTGTGTGTGCCACCGACAGTGCCCGTGCCCTTCAGCGCCACGACCTTCCTGTGCCAATCATGGGTATGATGT
CAGTTCATCTAATCCTTGAGCTGCAGTTAGCCCCAGATGAGGAAACAGACTGGAGCGATAAGTGACTTTCTTGTGGGCCAATATTGGACCCCCT
GGGTCCTCAGTTGGACCTCCAACACCAGAATGGACCTGGGTTCCATTCTGGTCCACAAACCCCCTTTGCGTTTGTGTCCCCTTCTCTGTAGGGT
GGCAGGCTTGCACTCAGAGCTTCCCAAGGACCTCCAAGGACTCTGCTGACAGCCTCTGCCAGGCCCATTTAATGCTGCAGGGCATTTAGGATTG
GGGGCCTCCCAGAGGTGGGATGGGGTGCCCTCTAGGGCAGCCCTCAGAAGCATAGGGCCAGGTGGTTGGCACCAAGCAGAGTGGGCAGAGGCAA
GAGGCAAAACTTCTAAGAGAGGCCACCAGCAGGAAATTCCACTTAGAACCCTGGCATTGCAAGTGGCAAGGGCCCAGGTGGGTGCCGGGGGACC
TGGTCTGGCCACCCCCAGCTAGGCGCCCCTTTAAGCAAGACCTCGGTCCCTCGAAAAGGGATGGCTGTGCCCACCCTATGGGGCCTGATGAGTG
GTGGGTGTGGAAGGGAGTACCTGGCACGGATTTTGCACCTGGAGGATGCTTGACACCTTAGGCTGCTTCTGTGGAAGAGGAGGTGAGGACCCTG
TCCTGGGAGCAGGTGCTTCTACGAAGCCCAGCTGTGGGGGTCTATGAAGCCTCTCCTGAGTTGGTCTCGGACTCATCTTCCCAGAGCCATGGGT
CTTTCTCTATCTCTGTGATACGGGGTTCAGTGCCCCCATCCCAGCAGGACCCCAAGTCTACACTGGGCAGCAACCCTGAGCCAGTGATAATAAT
GACTTATGGCCCCTGTTTACGGAGCATTTCCTGTGCACGTGCATATGGGCCTAATCTTGTTATGTCCTTCAACAACTCTGTGAGAGTAGTGACT
CTTAGTATCCCCCATTTTACAGATGAGGAAACTGACCCAAGTTCCTAGCCCAGTATCACGCAGCTAAGTAAGTGTCAGAGCTGAGGCCTCTCTG
ATGCATCGGAACCTAGATGCATTGGAACCCAGAGAGGCCACCATTCTATGCTGTGCCCTCATCCCTTGATTAAAGCAGAAATTGTTCATTTGGT
GACTTGGGGAGAAGGGAGCCCCATCCCTCTTCCTCAGGTTTGAGCGGGCCACCTGTCAGAGTTCAAGGCCTCTTTGATTGCTTGGAGGTGTTGG
CTGGGGCAGTCGGGACAGCTTTCCCCCTCCCTTTCCCAGTTCCAGGTCTGATGCAAACTCAAAGGAAAGGGAAGCCAGTCCCTGACTGGGCCTG
AGTCATTTGTTTCTGAATCCTAGTTTGAATCCTAGTTCCCGTTTTTATCATGGTGTGATCTTGGCTGGGCCCTTCCCTCTCTCTGGTCTCAGTT
TGTCTCTGTTCAAGTCCAGCACCAAGGGACTAGGTTGGCAGGGGCCGGAGGCCAGACAA
HUMAN SEQUENCE - mRNA
TTTTCACTGTCTGTGGACATTAAAAAAGCGAGCGGCGGCGGCCCGCGCCGCGGAGAGCGGGCGGCCGGGCGGCAGGCGGGCCAGCAGCGATCGG
GCGGCCGAGCGAGCGAGCAACGCCGGCGCAGCGCGGTGACCCCAGCCCCAGCCGGCGCGGAGCAGGAGCCGGAGCCGAGCGGATCTCGGCGCCC
TCGCTGCGCTCCTCCCGGCCCGAGCCTGCCCTACCCGGCGGTGCCGGCGGCGCGTCCTCCATCGGCGGCAGCGGCGCTCGCAGCGCCCGTGATT
TCGTACTACTGCTGGGGCTGCCACCTCCTCCTCCAGACGCTCTCAGCAGACTTGAGTCCTGGTCCTTCTGCAGAGGCCTGAGCAGGAGGAAGAG
GAGGAGGCCCGTTGGCGTCGGACCAATGCTGCAAGGGGTGTGAGGAGAGGAGCCGCTGTTTTTCACTGAGCTGCCATACCCCGAAAGCAGGATG
GAGCTGGAGTGAGGTGGAGGGGCCGCAAGCTGCTGACCGGCGTGTGGGACACTGGTGGTTTGCAGATCACTGAGGCTGGACAACGTTCATGGCT
CTCGGGTAGAACCTAGTGAAACGGCCAGAATGAATTCTATGGACAGGCACATCCAGCACACCAATGACCGACTGCAGTGCATCAAGCAGCACTT
ACAGAATCCTGCCAACTTCCACAATGCCGCCACGGAGCTGCTGGACTGGTGCGGAGACCCACGGGCCTTCCAGCGGCCCTTCGAGCAGACCCTG
ATGGGCTGTTTGACGGTGGTCAGTCGGGTGGCAGCCCAGCAAGGCTTTGACCTGGACCTCGGCTACAGACTGCTGGCTGTGTGTGCTGCAAACC
GAGACAAGTTCACCCCGAAGTCTGCCGCCTTGTTGTCCTCCTGGTGCGAAGAGCTCGGCCGCCTGCTGCTGCTCCGACATCAGAAGAGCCGCCA
GAGCGATCCCCCTGGGAAACTCCCCATGCAGCCCCCTCTCAGCTCCATGAGCTCCATGAAACCCACTCTGTCGCACAGTGATGGGTCGTTCCCC
TATGACTCTGTCCCTTGGCAGCAGAACACCAACCAGCCTCCCGGCTCCCTTTCCGTGGTCACCACGGTTTGGGGAGTAACCAACACATCCCAGA
GCCAGGTCCTTGGGAACCCTATGGCCAATGCCAACAACCCCATGAATCCAGGCGGCAACCCCATGGCGTCGGGCATGACCACCAGCAACCCAGG
CCTCAACTCCCCACAGTTTGCGGGGCAGCAGCAGCAGTTCTCAGCCAAGGCTGCCCCCGCTCAGCCCTACATCCAGCAGAGCATGTATGGCCGG
CCCAACTACCCCGGCAGCGGGGGCTTTGGGGCCAGTTACCCTGGGGGTCCTAACGCCCCCGCAGGCATGGGCATCCCTCCGCACACCAGGCCGC
CTGCTGACTTCACTCAGCCCGCGCCAGCCGCTGCAGCAGCGGCAGTGGCAGCAGCAGCAGCCACAGCTACAGCCACAGCCACGGCCACTGTGGC
AGCCCTGCAGGAGACACAGAACAAGGATATAAACCAGTATGGACCGGTCTGTTCCTCTTTCCAGATGGGTCCCACCCAGGCGTATAACAGCCAA
TTCATGAACCAGCCCGGGCCGCGGGGGCCTGCCTCCATGGGGGGCAGCATGAACCCCGCGAGCATGGCGGCTGGCATGACGCCCTCGGGGATGA
GCGGCCCTCCCATGGGCATGAACCAGCCCCGGCCGCCCGGCATCAGCCCCTTTGGCACACACGGGCAGCGGATGCCCCAGCAGACCTACCCGGG
CCCCCGGCCCCAGTCCCTTCCTATTCAGAACATAAAGAGGCCATACCCTGGAGAGCCCAACTATGGAAACCAGCAATATGGACCAAACAGCCAG
TTCCCCACCCAGCCAGGCCAGTACCCAGCCCCCAACCCCCCGAGGCCACTCACCTCCCCCAACTACCCAGGACAGAGGATGCCCAGCCAGCCGA
GCTCCGGGCAGTACCCGCCCCCCACGGTCAACATGGGGCAGTATTACAAGCCAGAACAGTTTAATGGACAAAATAACACGTTCTCGGGAAGCAG
CTACAGTAACTACAGCCAAGGGAATGTCAACAGGCCTCCCAGGCCGGTTCCTGTGGCAAATTACCCCCACTCACCTGTTCCAGGGAACCCCACA
CCCCCCATGACCCCTGGGAGCAGCATCCCTCCATACCTGTCCCCCAGCCAAGACGTCAAACCACCCTTCCCGCCTGACATCAAGCCAAATATGA
GCGCTCTGCCACCACCCCCAGCCAACCACAATGACGAGCTCCGGCTCACATTCCCTGTGCGGGATGGCGTGGTGCTGGAGCCCTTCCGCCTGGA
GCACAACCTGGCGGTCAGCAACCATGTGTTCCACCTGCGGCCCACGGTCCACCAGACGCTGATGTGGAGGTCTGACCTGGAGCTGCAGTTCAAG
TGCTACCACCACGAGGACCGGCAGATGAACACCAACTGGCCCGCCTCGGTGCACGTCAGCGTGAACGCCACGCCCCTCACCATTGAGCGCGGCG
ACAACAAGACCTCCCACAAGCCCCTGCACCTGAAGCACGTGTGCCAGCCGGGCCGCAACACCATCCAGATCACCGTCACGGCCTGCTGCTGCTC
CCACCTCTTCGTGCTGCAGCTGGTACACCGGCCCTCCGTCCGCTCTGTGCTGCAAGGACTCCTCAAGAAGCGCCTCCTGCCCGCAGAGCACTGT
ATCACGAAAATCAAGCGGAATTTCAGCAGCGTGGCTGCCTCCTCGGGCAACACGACCCTCAACGGGGAGGATGGGGTGGAGCAGACGGCCATCA
AGGTGTCTCTGAAGTGCCCCATCACATTCCGGCGCATCCAGCTGCCTGCTCGAGGACACGATTGCAAGCATGTGCAGTGCTTTGATCTGGAGTC
ATACCTGCAGCTGAATTGCGAGAGAGGGACCTGGAGGTGTCCTGTGTGCAATAAAACCGCTCTGCTGGAGGGCCTGGAGGTGGATCAGTACATG
CTTCTTCTCTGTCTCCCCTCCTCCCTCCTTCCTGCCCTCTTTCCCCCTCCCCCTCTGTCTGTGTCTTCCTTCTCTGTATCTTCTTTCTGTATCT
TCCTTTCTTCTCTCTATTCCGCTCATGGCCTTGCCTCTCACTCTTCCTCCCTTTCTGTGTCTTTCTCCACTGACAAGACTCTCTCCTGCCTCCT
CTGCTCACATTTCCATCCCAGTGCTCTTCCTCTCCTGAGGTCTTCTCTCCCTCCTCCTCCACAGTAGAGCCTTTCCCGAAGCTGCATTCCGGAG
TGCCCGCGCGACAGAGCCTGCGAGGTGFGCTGCGCTGGCGGCTGGGCCCACCAGAGGAGCCCTGTGCAGCCCTGAGTTGTTGGGGTGGGGGATT
GGTGAAGAACACAGAGTCTAGGGTTGGGGTCTCCGGGTTCAAATCCCACCCCACCCCTTGTTAACTGTGTGACCTTCCGGTTAGTTCACATTGA
TTTCTGTGCTGCATCCATAAAATGGGGTGATAATTTTACCTGCCTCCACAGTAAAGCCCTTCCCGAGGCTTCATTCTGCAGCCCTGAGACAGTG
GGGTGGGGGATTGGTGAAGAACACAGAGTCATGGAGTTATTGTGAGAAGTCAGTGAGATAACCTATGTGAAGCGCTTTAGCGGAGGGAGGGGCT
TGGCCAGAATTAGCCTTTTTACTTTCTTAAGAGACAAGGTCTTGCTTTGTCACCCAGGGTGGAGTGCAGTGGCTCACTGCAGCCTTGACCTCCC
AGGCTCAACTGACCCTCCCACCTCAGCCTTCTGAATACCTGGGACTATAGATGCATTCCAGCATGCCTGGCTAATTTTTAAAATTTTTATTAGA
GACAAGGTCTCACTATGTTGCCCAGGCTGGTCTCAAACTCCTGGGCTCAAGCAGTCCTCCCATCCCAGCCTCTCAATGTGCTGGGATTATAGAT
GTGAGCTGCCACACCCAGCCCAGGAATTAGCTTTTATTTGGGAACTCTTGGCCATGCCTTTCATTTTTTGCGGCCTTCCTGAGATGCATCTGCA
GTGAGTTTCTGGAGTCCTGGGGGTCAGGCCTGAGGCCAGGGAAGGGTGGGAGCTGGCTGCAGACCCCCATAGAGCTGGCATTTCCCGCTCCCCA
CTCTCCACCCCTCAGCCTTCCTCCCCTAGAGCAGGGTCTCCGCTCTGCCTGTCAAGACATGTGGCGTCCTTCTGTCTTAGTGGGCCCATCTCAG
GCTGTGAGGACAAGCCTGCCACCCTGGGGTAGTAGCGGAGACACAGATCCCCAATGCTGGAAAAGTGCTCAAGACTCTGGCCCGCAGACCTTTC
CCAAGCAACAGACCAAAGGCAGAGCCGCTCCTGCTGAGTTGGACTAGACACCCCCTTCTTCTACCCTCCCTCACCCCCTAGATGCCCCAAAGGC
TATAATATACCATGGACTCCAGCTACAAAACCACTGGTCCAGGTGCAGCATGTTGGCAGCTCTGCCATTTATCACAGGGACTTCTCTCTGAGCC
TTGGCTCCCCTCCTGTGTGGCCCCAACGTTGGGCTTTGGGATTCTCCCTGAGTTTGGGGATTGTCCCTGTAGTCAGGGAACGGCTGGGTGGCCA
CAGTTTCCCAGCAGACATCTGCCCTCTGGGTGGGGAGGGCGGAAGGAAAACACCAGACACAGGCAGGGCTGGGGCTGGGCGGGTGCTCTGCCCC
CTCCCTCTGCTCCCGCTTCCGCCCCGACTCTGCTGGGCTTCCCACCCAGCAAACCCCTGGCACATCCAGGAAGCAGAAAACAAGTGCTTGGGGA
GGGCATTCTTCTGGGTGAAGAAGGGCAGCCGGTCACTTGTCCTTCAGAGACACAGAAGCCTGGGCCACATGACTACCTGACCCACAAGTGGCTG
TGCGTGTGTGTAGTGTACGTGTGTGTGTGTGTGTGTGTGTGTGTATATCCCTGTCCCTGGCCAAGGAAACCTTAGCCATCACGTGGCTGGTTTC
TCTCGCTATTTGTCCAAGTAAGCAGTTCAGGCCAGCTGCAGCCAGGAGGCCGTGACACAGAGCCAGGGAGAAGGGCCACATTGGTGTGGGTGGT
GGCAGCGAGGATCGCCATGGGCATCAGGGAGCCCCGAGCTGGGTCCTAGAGCTCTTGGCCCTCAGAATCTGGTATGGGGAGGCCTGGGCTGGGG
AATGGCCTTGTTCTGATCCTCTGCCCTCCCTGGACGACTGACCTCCCTGCCCCAACCCATCTGTCAAGAAAAGGCACAGAGCTTGATAACAGAG
AACTCTCTCAGCCCTAAATAGAGAGGGTGGGCACCTTCTGACCAGGGGGAGCCGAGAAAGACAGAGTCCTCACCAGAGAAAGGGGCACACATGG
GCACAACCACCATGTGTATGGCCCCTGGAGTGTGCTTTAGAGCCCTGGCCTCCGTGGCAGGAGTGTGGAAGCCAGGCCTCCCGTGTGCATCCAT
CAGGCCACTGATTATTAGCACCTACTGTGTGCCACCACCTGGGCTAGGCAGGGATAGGGGTTGTCAGGAACACAGACCTTCTGGAAACTTCGTC
AAACCCTCGCCTCTGTGACATATTTTCTTCCCATTGGATGGGCAGCATCACCTTTCATTGCAAAACCCCTGTGATCTTGTCATGAGCTGGCCAT
TTCCCACCCCACTGCCATTCCAGGAGGATGCAACCCCTACATTGTTGCAGGGCTCAGGAAGAGCAGTGAGAGGCACCGCCGAGCCTCTCATGGA
CCAAGCAGGTGTTTCTAGCCAGGGAGAGTGTCCCCTTCCTCTAGGGAGGTGTGAGCCAGACCCTGGGCTTCCAAAGCAAATGTGTGCATGTGGG
CAGAACACAGAGGCCCTGGGCTCGCACACCTGGGGGCCTTCTCCTAGCCCACATGGGGAGCAGAGCACCAGGGGAAGTCGCAGCTGCAGAGTGG
GGCCTCCCCAGGCCTTCATCGAGGTTTGTCAGCCTGCATACTGTGTCTTGCATAGATGACACCATCTCTGGGGGCCTCGAGGAGGAGGGCTGTG
GTGGCTTTTTGGGGCCATGCTTCTGAGAGCCCACCTGCATTCATGTGTCTGTTTGTCCACCTCCTCCCTGCTTCTTCCCTCTTTCCTTCCTTTG
ACAAACATTTCCTGGCAGCCAAGTCTGTATGGGGCCCACGCTCCACAAAAGATACATCCCTCACCCTGCATGATCACGGCCGAACTGGAATCCA
GCAACATTCCAGGGTGGGCCCAGGGCCAGGCCCAGGTAAGGCCTAGGAGAGAGCTGGCTCTTGGCTAGGAGGCAGGGCATGGCTGACCCCACAG
GCTCCCCTGGGTTCTGAGGAAGCTTAGGCGTACTTCCCCAGGATAGCCCAACGGCAGAGCCCACCAGGTGCCTGAGACAAGGGGGTGTAGAGAG
CCAGCATCAAGGCCACACAGAGCATCATCAGACCTTGTCTCTGTTGTGATGAGGTCTAGGGTATTCAATCTGGAAGAGTCCCACAGGCTCGTGT
GCTCAGGCTGTTCTAGGAAACGCCTGTCCGGAAAAGGGGTGGAGCTGGGTGGCCAGGGCTGAGGGTGACCCAGATCCGCCATCATGCCACCTAC
AACTGAATGTGGATTCTGGGCCGGGGTTTTTAATAAAGCTGCTGGGTCAGCATCCCAGAATGCTGCCTACGCCTGTATGTCCCCAGCCTCAGCT
TGCATATCTCTGGGGTCACGAGGCTTACTACTGCCGTCTTGTAGTTCCTCCTGGGACATGGGGTCCTGGCCTCCCACCAGGCTGACGCTGACTT
CTGTCTTCCACCCTGCTTGTCTTAGGGCCACAGACTTTAGGCCCTTGGCAGTTTCCAATTGAGTCCCTCCCGGGCTTTCCTCCCTGCAGGCAGA
GCATCAAGCTGGGTCTGTACGTGGCCCCGCCAGGGCCTGTTTGATTGGGGTAATCTGGACAAGTCATCAGTGGCCTGCGCTGGCAGCCAGGGTG
TGGCTAAAGTCAGTTCCTGCCTAAGCTGGGCTGACCCACCTTCTCACCACTCAGGAGCTCCCGGAGCCAGGAGGGACTGCTGTGAGTTTGCCTT
CATGAGGAGGCCCTTGGGCTGCCTTTGGTACAGGCAGGAGACCTGGATCCCTCCTCTGTCCCTATCTCTGTCCCTGTCCCTGGGGTAATCTCGA
GCAAGGCTGGGCCCCAGGCAGGCCCACACCCCCTCCCATCTGTGTTCATCCCCAGCACTAAGCCAAGAGGCTTTTGTCCCTGGTGGGCTGTCGG
CCAGCTGGAAGAGTCCTCCTTGCTCTCTGGCCTATTTGCTTTCTGAGTCCCTCTGCCCTGGCAGAGCATGAATGGGCAGGAGAGGGGACTGGAG
AGGGCTGAGGGCCTCTGTTCAGATCTCTGCCCTCACTGGGTCGGGCTGCCAACCTCTGCACTCCCACCATACCCCCAACCTGTGGACAGGTGAG
CCTCCAGTTCCTGAGGCCACAGGGAATGGGCTGGATTCACCTGGAGGCCATGGGTAGCCTTGCAGCCCCTGGGCCAAGCTCCCCTCCCACCCAG
ACTCCTCCAGACACTTCAGGGGTTCAGGCATGCCAAGTCAGGGTGGGGTGAAGGGGTCTTTCTCTAGGCGCTGTGCCTGTGGTTGACATCACGT
TGTCCTCATGTGGGGCTGCGTGGCCCCGTCGGGTGGCACGCCTTCTGCAAGAGCTCGCCCTCCAGGAAGTTCTGCATTCGAAGCCTGAACTTGG
CCTTGGTTCACTCTTCCTCTCATCCTGGTCTCTTGGGCATGACTTCTCCCAGGGGACACTCCTGTCTGCTGACTTGGAAAGGCAGGGATTCAAT
CAGGGGCCCCCGCTCAGCTGTGTCTGAAGTGTGCCCTCCTGGGAAGAAAACCCACCAGCACTTTTGTCGAGGCCTCACACTGGAAAATGAACGG
TGGGAGGCCTAGAGGTAAGAGGCCAAAGGCCACTAAGCAGGCCAGCGTGGCCTGCCCACTCCAGCCCCCAGAAAGCATTGGTGCTTCTGAGGAG
GCTTGCTCGGCCCTCCCTCTCCCCTGGGCTGGGTGGGGCCAGTGGGACAGGAAGCCAGGGCGGGGCTGGAGGATGGGGCTCAGGGTGGCCTGGG
CCTGAGCCAGGGATGAGGCGAAACCCAACCCTGGCCTGGCCCCGGGCCGCAGCCGTGCAGGTCACTAGGCCTCGGCGAGTGGCCACCCGCCCAC
CCAACAGGAAGCCCTGCCCAGACACCACCTTTGCCCCACCCCGTATTCTCTACGGCTGGCACTTCTCCTATTTCCCATGAGAGCTGCTGCCAGG
AATCCTGCCCTCTTGAGAAAGGAAGCCCGACCTCGGGCCGCCCTGCCTCTGGCCTGCGCTGACCCCCTCCCTGGGCCTCCCCGTGCTCGCAGCT
CTGGGCCCACGCCCGTCCTGCACCCCCCACACATGCTAATGACTTATCTCCTCTCAGACAAAGCCTGTGACCCTTGGGGACCTAGCAGGAGAAA
CCTCACAGTGGGACTAGGCCTGGGAGCCAGGAGGCTGGGGCTCTTGGTCTGGGATCCTCCACCAACCTGCTGGTGCACCTGGGCAAGTCACTCA
GCCACTCTGGGTCTCATCTGTAGAAAAACAGGGAGAGGCAGACGCTCTGCCCAGCAATGAGCATCCCATGGGATAAAGGGTTTGGGGTGGGTTG
AATGTGTGCCCTGTGAAGACAGCCAGGCTTGGGGGGTGATAACACAAAACCTCCCCATCCACCCCTGTCTGCCTGGAAAGGAGCCCCTTCTCTC
ATCTCCCAGAAGCCGGGCGACGGAGCATGCCTCCGTCTCTCAGCTCTGCTCAGGGAGCTGGGCCAGAAGCAGGGATGGGGCTGTGCTGAGGGGC
AGGCTGTCCCACCACCCTCCCAACCCCATCCTCTTCCTCCCTCCTGGTGGGTGCTCTCTGACCCATCACCCACCTCCCCAAGTAAGCCCAAGCC
CACCTCAGTATCAGCAGGGGTAGCAGCTTAGGGAATCAACTGTGTCTTAAAGGGGGCCAAGCAGAGGGGTCAGGGCACCACGGGGAAAGACCAG
AGTCGTTGTGACATTGGATGGGTTAGACAGATCCGGGGGCCACCTTGAGCTCCTGGGCGAGCTGGAAAGCTTGTCAGCAGCAGGGAAGACCAGG
CACCTGAGCAGCCAAGCCCCTGCCATGCGGTGGCCACAGCTCAGACTCTGGTGGCCTTGGACACATTGCTGCTCTGAGCCTCAGTGTCTGCATC
TGGCAAGTGGACCTTTCAGAGCCAGTGTCTTAGGGTGCTGTGAGAAGTCCTTAAGATTTGGGGACACAGTGCTGGGCCCCCTGCGGCCCTTCCC
TTTGAATCAGCCTTTTCCCTGTGAGGGAAACATTACAGGTCAGGATTAGGACTAGATTTCGTAGGCAGTGGGGAGCCACTGAAAGTTCTTAGAG
GAGTAGTGAGTTCATGGGGAGAGGAACCGGCAGCAGACAAGCCTGGCACACTCATCCGGTGTTGGAGCCCTGATTCAAGAGGGGTCATGGGGCA
AAGAAAGTTAGCCCAAGTGGGTGTCAGCACCAGCTGGGCTTGGGAGCCCTCACTTTTCCTCCTGTAGCTGGGTGCCTGTCCGCAAGGCTGGAGA
GGCGCTGCTCACAGCCAGCACTGAGGGTGGCACCACCACCCCTCCTTCTTCCTCTGGTGCCCCTGAGTCCCACCGGCGTGGGGTTGTCTGGGTT
TGCCACTCAGGCCCGGGTGGGCCCCACTCCCTCTCCCTCCCTCCATCGCTTTCTAGCTCTGAATGGGAGTGGACTCTGGAGGCTGCAGTGGGCT
GCGGGATGTGAGCCAGGGTGGGGGCATTGGCGGTGGGTGGAGTCACGGGCTGGGTGGCCATGAGTCAGGAATTGCTGGGCCTGTGTGTCATCTG
TGGGGAATCCTGAATGCCATCCAACACTCCGAGTTTGAAGAGGTCACCATCGATCCCACGTGCAGCTGGCCCCCGGTGCCCATCAAGTCGGACT
TACACATCAAGGACGACCCTGATGGCATCCCCTCCAAGCGGTTCAAGACCATGAGTCCCAGCCAGATGATCATGCCCAATGTCATGGAGATGAT
CGCAGCCCTGGGCCCCGGCCCGTCCCCCTATCCCCTCCCCCCTCCCCCAGGGGGCACCAACTCCAACGACTACAGCAGCCAAGGCAACAACTAC
CAAGGCCATGGCAACTTTGACTTCCCCCACGGGAACCCTGGAGGGACATCCATGAATGACTTCATGCACGGGCCCCCCCAGCTCTCCCACCCCC
CGGACATGCCCAACAACATGGCCGCCCTCGAGAAACCCCTCAGCCACCCCATGCAGGAAACTATGCCACACGCTGGCAGCTCTGACCAGCCCCA
CCCCTCCATACAACAAGGTTTGCACGTACCACACCCCAGCAGCCAGTCAGGCCCTCCATTACATCACAGTGGGGCTCCTCCTCCTCCTCCTTCC
CAGCCTCCCCGGCAGCCGCCACAGGCCGCTCCCAGCAGCCATCCACACAGCGACCTGACCTTTAACCCCTCCTCAGCCTTAGAGGGTCAGGCCG
GAGCGCAGGGAGCGTCCGACATGCCGGAGCCTTCGCTGGATCTCCTTCCCGAACTCACAAATCCTGACGAGCTCCTGTCTTATCTGGACCCCCC
CGACCTGCCGAGCAATAGTAACGATGACCTCCTGTCTCTATTTGAGAACAACTGAGGGCCACCCGGTCGGGGCCATCCCTCCACACTCTGCATC
CTACCCCACCTACCCAACACACTTTTCCACCTGGGAGCCTGTGCCCTCAGACCGCCCCGCACCAGAGCCACGGGCTGTGGGGCGGGGAGCCCTC
CCCCGCTGCAGCCCTCTCAGAACAGAGGGGTAGGGAGGGTGCACCAGTGCACCAGGAAGGCTGTGTGGGTCTCGAGCCCACGTCCCACCTCCAC
ACCCTTGGCTTGGGCCCATGCCCAGCGCAGGCCTGAAGACCACCCTCCCGAGAGGAACCAGCCCGGTAAGAGCGCACACGCTGATGCGGCTTCC
CGGTCCCTCCGCGTGTGCCGATTCCAGATGACCTTCCAGTGTCCCCAAGGTTCTTCCATCTTCTAGACTGTAACCCTGCCTCCCTGCTTCCTGG
TCCAGAGCCTCCCTCCAGTGACTGTGGACCCTGAGAAGGCCCCCGGGCCCCACCATGGGCCCCGAGCCTTGGAGGAGCACTGGCAGTTGGTGGC
AGTGAGACCAGCCACCCACCACCACCCACCACAGAAAAGCACAAACCTCTGGGAAAGACAACGTCTCTCGGGGGCCAGGGGTCATCGGTTTGAC
CCCTGACCTATAAGCCAAGATACCCCATAAACACACTCAGAAAGCAGAGAAAAAGGACAAGAGTCTGTGTTTGAGAGGGGGTCTGCCATTCCTG
CTTGGGGACTGGTGGGGAAGAGGGCCAGGACATCTTCTGAGCCAGGACGTCCCTGAGGCTCCACCTCCAAGCTCAGACAGGGCCCAGGCTTGGG
GAACAGAGAGAGCAGGTGTACACCCAACCAAAGTGATTGTGCCCTTGGTTGGGGGGCGCGGGCATATAACCTGTCAGAAGCAAACAGGAGCGGC
AACTTCTAACTTTGCTCCAAGCCACTCTCTTTTTAAACAGCAACAATTTAAAGCTATGAAGTCACCTGGAGAAAAGGAACGTTGCTCTTGGACA
GCAAGCAAACCATTTCTCTCCGTCTGTTCTCTTTTTCTCCTAGTCCCTCTCCTGCCACCTCTCCAAGACTTCCGTGGGACACCCACTTCCCTCT
GTCCTAGTTCTCTTTGTCCAATCAGATGGCAAGGGCAGTGCGTGGAAAGGCCGGGGAGGTGCAGAAACCAGAGCCCAGGGCAATGGTGTCTGTC
CAGCCCCTCCCTCTGTCCCTGTGCTCCAAGCTGCCCCCGGCTGCACCCCAGGCCATGGACATGTGCACCAGTATGTACCTGCAGGCATCAGGGG
GAGGGGGGCGTGTTTCTGGGCCTGCCCCAGACACTGCCCTTGGCTGCCAGCCTACCCTGCCTGCACTCCTCCACCATCACAATCTCACCCAAAC
TCCTGCTCACTCAAGCAAAAGCAGCCTCTGGCCTTCCCTCCACCGCTTTGCTCCATCTGGCTTACCACTCTCCACGGCCTCCTGGGGAGCCTGT
CCTGTGTTCACTTTGTTTCAGGCTGGTCTGTGCCCCCTGAGCCACATGGCCTAGGGTGATGCCAGGTTGTCCCGTCACTGGGGTCCCATCTGTA
AATTCTTTGCGCCCTTCCCGGCTGCTGCCTGGGGCCCTTTCCTGCTCTCCCGPCCGCTGTGGGTGGTCCCCAGCACTCCTCTGTGGGTTTTACC
GGAAAGGTGGCCCCAGCTGTTGACTTCCAGTCACTGTCCCAGACGGCACAAGGTTTTCTGTAGGAAAGCTGCCATPGCCCCGGCCCCTTTTCTT
CCTTTGTCCCGTTGTCGAGGTTTTTTCAAATAGCGTGTTGTTCAGTATGCAAATCAATTATTTTAAGAATCGCTTTTGTAAATATCTTTGTGAA
TATTTTAGTATCGTCTTTGATAATATTCAACATTTTCATGACCTGGTTATAGCCTTTGCTGGTGTTTTTAAAATACCTGGACTCAATGACAAAG
ACCGAGTCTTCTTTTTTTTTAAACAAAAACAAAAAAAGCAACCAGGGCTATTTGTACAGTTGAAGGGGTGAACAGAATGGGCGGCTGTGCTGGG
AGTTGGAAGACCGGGCAGCCCGCTATTTAGAGCCATCCCTCAGTCAGCTGGCAGGGACAAGCCAACGCCAGGTACCATGTGGCCACCCTTGCCC
AGTGTCTGTGGCCTGGCAAGTGGCCACGCCCTGTGTCAGACCATCTGGGAATTAAGCTCCAGACAGACTTACAGATGCCTTCCTTAGGAGTTCT
TGCTTCTTGCGTTGATACTTTGCCCCAGAAAGGCCTGGGATTCATTCTCGTTCTTATCAGGGTGTGTCCACACTCTGCTCACAGGTGGATCCAC
GGCTTTCCAGTGCGGAGAGTCGAGATGCTCCCTCCAGCCCAGCCCCCGGGCACCTCCTGCAACCATCTCTGGGCTCAGCACCTGAGGCGGGTTT
CCTGGGTCCCCTCTCCAGCAAGCCTCCACCAGCAAGCTCGGCCCAGAGCTTCCCTTCCGGCTGGCTCTGAACCGTGCGTGGTGCCTACAGCCTG
CAGTCTGGAGACAAGCTCTTCCGGAGTGCTCTGGGAGCCAGGCCAGGGTGTGAGGGAGGTGCAGAGGCATCCGGGGCGGGAGCAAGCCCCAGGT
TGTGACAGGTGCAGGTAGACAACGCCCATAAACAGAGATGGTCCTGAACTCTGGAGAGATCCTTCCCTGATCCTTTCGGACGACTACTTGGAGC
CATAAGTAACCTCAGCAAAAACGAGGCCTCTGCAAGCCACTTTTCCATGCCAAGCATCCACCCGGCCCACAGGCATGTTTCTGCCGCCACTCCG
CAAGATGGACAGGGAGCCAGCAGGCAGGCGGGAAGGGCCAAGTACAGGCAATCACCCCCATCTTCTTGGTTTGAAGCTTTATCCATGTATCATG
TTCCGTGTAGCCATTTTATTTTTTAAGAAACTGCTAATACTTTCTCCCTAATGGAAGCCCTGATCCCCCAGAGAGCTACAGGTCTGCTCCCGAC
GGGCCTCGGGCCTGACCCGTCCACACAGGGCCGTGTCAACAGCAGCGACTCAAGGGACGTGTGTACATATGTAAATGAGAAATAGAGACGTGTC
AACAGATGCATTCATTTCTCTTGGAATGTGTATTGTTTTTATTTTGCGAAACAAAACAAAACAAAAAAAAAAGCTTGGAACTCCATCACGTGGA
AAAACTAGATCCTGTTGGTTATAGCATTTGTGAGTTCTCCACGTCTGTCTCTCTCGCTCATGTAATATACTCTGACCCTGAGTGGAAAGGGGTT
TTTGTTCTGTTTTTATTTTACCTACATGTACTATTTAGC1TCAGTGTACTAGTCCTGCCACCTGTGTATTTTTAGGGTGCTATGGAAATAATGA
AAAGAAACGGGGATTTCAGAAGAAAATTGTAACCAAATTCATACTTTGTATAATTTTTGATATCATGATCACAGGTGATTCACACGTACACACA
TAAACACACCCACCAGTGCAGCCTGAAGTAACTCCCACAGAAACCATCATCGTCTTTGTACATCGTATGTACAATGCAATCATTTCATACTTTA
AACTGGTCAAAAAACTAATTGTGATTTCTAGTCTTGCAAAGCTGTATGTAGTTAGATGATGTGACAACCTCTAATATTTATCTAATAAATATGT
ATTCAGATGAAACCTGTATATTAGGTGTTCATGTGGTTATTTTGTATTTAAAGATCAAATTATTTGACTATTGCTAGACATTTCTATACTCTGT
TGTAACACTGAGGTATCTCATTTGCCCATGTTAATTTTTTTCTAAATAAATTGACAAAAACAAAGGTT
HUMAN SEQUENCE - CODING
ATGAATTCTATGGACAGCCACATCCAGCAGACCAATGACCGACTGCAGTGCATCAAGCAGCACTTACAGAATCCTGCCAACTTCCACAATGCCG
CCACGGAGCTGCTGGACTGGTGCGGAGACCCACGGGCCTTCCAGCGGCCCTTCGAGCAGAGCCTGATGGGCTGTTTGACGGTGGTCAGTCGGGT
GGCAGCCCAGCAAGGCTTTGACCTGGACCTCGGCTACAGACTGCTGGCTGTGTGTGCTGCAAACCGAGACAAGTTCACCCCGAAGTCTGCCGCC
TTGTTGTCCTCCTGGTGCGAAGAGCTCGGCCGCCTGCTGCTGCTCCGACATCAGAAGAGCCGCCAGAGCGATCCCCCTGGGAAACTCCCCATGC
AGCCCCCTCTCAGCTCCATGAGCTCCATGAAACCCACTCTGTCGCACAGTGATGGGTCGTTCCCCTATGACWCTGTCCCTTGGCAGCAGAACAC
CAACCAGCCTCCCGGCTCCCTTTCCGTGGTCACCACGGTTTGGGGAGTAACCAACACATCCCAGAGCCAGGTCCTTGGGAACCCTATGGCCAAT
GCCAACAACCCCATGAATCCAGGCGGCAACCCCATGGCGTCGGGCATGACCACCAGCAACCCAGGCCTCAACTCCCCACAGTTTGCGGGGCAGC
AGCAGCAGTTCTCAGCCAAGGCTGGCCCCGCTCACCCCTACATCCAGCAGAGCATGTATGGCCGGCCCAACTACCCCGGCAGCGGGGGCTTTGG
GGCCAGTTACCCTGGGGGTCCTAACGCCCCCGCAGGCATGGGCATCCCTCCGCACACCAGGCCGCCTGCTGACTTCACTCAGCCCGCGGCAGCC
GCTGCAGCAGCGGCAGTGGCAGCAGCAGCAGCCACAGCTACAGCCACAGCCACGGCCACTGTGGCAGCCCTGCAGGAGACACAGAACAAGGATA
TAAACCAGTATGGACCGGTCTGTTCCTCTTTCCAGATGGGTCCCACCCAGGCGTATAACAGCCAATTCATGAACCAGCCCGGGCCGCGGGGGCC
TGCCTCCATGGGGGGCAGCATGAACCCCGCGAGCATGGCGGCTGGCATGACGCCCTCGGGGATGAGCGGCCCTCCCATGGGCATGAACCAGCCC
CGGCCGCCCGGCATCAGCCCCTTTGGCACACACGGGCAGCGGATGCCCCAGCAGACCTACCCGGGCCCCCGGCCCCAGTCCCTTCCTATTCAGA
ACATAAAGAGGCCATACCCTGGAGAGCCCAACTATGGAAACCAGCAATATGGACCAAACAGCCAGTTCCCCACCCAGCCAGGCCAGTACCCAGC
CCCCAACCCCCCGAGGCCACTCACCTCCCCCAACTACCCAGGACAGAGGATGCCCAGCCAGCCGACCTCCGGGCAGTACCCGCCCCCCACGGTC
AACATGGGGCAGTATTACAAGCCAGAACAGTTTAATGGACAAAATAACACGTTCTCGGGAAGCAGCTACAGTAACTACAGCCAAGGGAATGTCA
ACAGGCCTCCCAGGCCGGTTCCTGTGGCAAATTACCCCCACTCACCTGTTCCAGGGAACCCCACACCCCCCATGACCCCTGGGAGCAGCATCCC
TCCATACCTGTCCCCCAGCCAAGACGTCAAACCACCCTTCCCGCCTGACATCAAGCCAAATATGAGCGCTCTGCCACCACCCCCAGCCAACCAC
AATGACGAGCTGCGGCTCACATTCCCTGTGCGGGATGGCGTGGTGCTGGAGCCCTTCCGCCTGGAGCACAACCTGGCGGTCAGCAACCATGTGT
TCCACCTGCGGCCCACGGTCCACCAGACGCTGATGTGGAGGTCTGACCTGGAGCTGCAGTTCAAGTGCTACCACCACGAGGACCGGCAGATGAA
CACCAACTGGCCCGCCTCGGTGCAGGTCAGCGTGAACGCCACGCCCCTCACCATTGAGCGCGGCGACAACAAGACCTCCCACAAGCCCCTGCAC
CTGAAGCACGTGTGCCAGCCGGGCCGCAACACCATCCAGATCACCGTCACGGCCTGCTGCTGCTCCCACCTCTTCGTGCTGCAGCTGGTACACC
GGCCCTCCGTCCGCTCTGTGCTGCAAGGACTCCTCAAGAAGCGCCTCCTGCCCGCAGAGCACTGTATCACGAAAATCAAGCGGAATTTCAGCAG
CGTGGCTGCCTCCTCGGGCAACACGACCCTCAACGGGGAGGATGGGGTGGAGCAGACGGCCATCAAGGTGTCTCTGAAGTGCCCCATCACATTC
CGGCGCATCCAGCTGCCTGCTCGAGGACACGATTGCAAGCATGTGCAGTGCTTTGATCTGGAGTCATACCTGCAGCTGAATTGCGAGAGAGGGA
CCTGGAGGTGTCCTGTGTGCAATAAAACCGCTCTGCTGGAGGGCCTGGAGGTGGATCAGTACATGTGGGGAATCCTGAATGCCATCCAACACTC
CGAGTTTGAAGAGGTCACCATCGATCCCACGTGCAGCTGGCGGCCGGTGCCCATCAAGTCGGACTTACACATCAACGACGACCCTGATGGCATC
CCCTCCAAGCGGTTCAAGACCATGAGTCCCAGCCAGATCATCATCCCCAATGTCATGGAGATGATCGCAGCCCTGGCCCCCGGCCCGTCCCCCT
ATCCCCTCCCGCCTCCCCCAGGGGGCACCAACTCCAACGACTACAGCAGCCAAGGCAACAACTACCAACCCCAPGGCAACTTTGACTTCCCCCA
CGGGAACCCTGGAGGGACATCCATGAATGACTTCATGCACGGGCCCCCCCAGCTCTCCCACCCCCCGGACATGCCCAACAACATGGCCGCCCTC
GAGAAACCCCTCAGCCACCCCATGCAGGAAACTATGCCACACGCTGGCAGCTCTGACCAGCCCCACCCCTCCATACAACAAGGTTTGCACGTAC
CACACCCCAGCAGCCAGTCAGGGCCTCCATTACATCACAGTGGGGCTCCTCCTCCTCCTCCTTCCCAGCCTCCCCGGCAGCCGCCACAGGCCGC
TCCCAGCAGCCATCCACACAGCGACCTGACCTTTAACCCCTCCTCAGCCTTAGAGGGTCAGGCCGGAGCGCAGGGACCGTCCGACATGCCGGAG
CCTTCGCTGGATCTCCTTCCCGAACTCACAAATCCTGACGAGCTCCTGTCTTATCTGGACCCCCCCGACCTGCCGAGCAATAGTAACGATGACC
TCCTGTCTCTATTTGAGAACAACTGA

TABLE 10

MOUSE NOMENCLATURE
ICSONM Lfng
Celera mCG14497
HUMAN NOMENCLATURE
HGNC LFNG
Calera hCG18436
MOUSE SEQUENCE - GENOMIC
GGAACCAGGCATGGCAGGAAAGCCACCCTGGAGAGCCCCTGCCAGGCGGCCCACTGCTTCTCAGCAGCCTCACTGTGGGTGAGACTCTGTGGGA
TCCCACGTCCTGTATGACTTGCCCAGTTTATCAGCACCCGGGCTTCTGCTCAGTGCTCAGCATACGCCTATTTCTCCTGAGCTGTGTGTGCTGA
TGGTTTCCATGCTGGGGAGGGTCTGTGAGGAGGTCCTGTGAAGAGGGGACCGGGAGCTGAGGGATTCTAGGCAGGGGCTCTACCACTGAGCCAC
ACCACTGAGCCACGCCCCCAGCCCCTCACTGGGGGATTCTAGGCAGGTGCTCTACCACAGTGCTACAGCCCCAACACATTTCTGCTCAATTGTG
ATAACCAAATCTGTGTGTTGGCATTCATTGTCAGATGTCTAGGACCCAAATCACCTTGAATGAAACCCACCGGGCAATAGGCTGGGCCCTGCAG
ACAGCAAGGAATAAGGTAGCCCTGGCTACGGAGTGGGAGGAGCTTCAGAAGATCACCTCCCTTCCATGTTAGCCTCGGCTGGTCTGTCTCACCC
TGCGGCCTCCTGTTTCCTGTTTTGGTGAATCCCTGAGCCACCTGGCAATGCCAACTCCAACAGTGCCTACACCTCCTGCAAGACTAAACTCTTC
CTTTGATTAATCCCTCCTAAGGAAGCAAGGAAAGACAACAAAAGCTGGTCTAGGCAGTTTCCCAGCCCTTACAGCTTTCTCCATCTTAAGGCTC
CAAGGACACAGACCTAGAGGCTCAGGTGAGCACACTGACTAACAAGGACTTGCCATATCCCGCATTTACTTCATCTGCTGTGCCTTCCCTGGGC
TCGCAAGCCTGCTGTGGCTCTGTCCTCTCCTCCCAGGTGGCATCACGGCCCTGGACTTTAGGCTCCAGCTCATCCTGGTTTCTATTGCATTGGG
TGCTACCTTCACCCATGTCTAAGCATACAACCTCTCCAGACAGGACAGGACCCCTCTGCCTGCCCCAAGTGTCTAGTCCCTATGATGGGTCAAC
AAGATGATTCAACAGGTTAAAGTGCTTGCTGCCAAGCTTAAAGACCTGAGTTTGATCGCTGGGACCACCATGGCGGGAGGAAAAGCCAACTCCT
GCAAGTTGTCCTCTGATCTCCACATACATACATGGCATGCACATACATGTATACAAGTAAAGAGATCTAAAAACAACAATTTTTCTTTTTAATT
CACCACATTAAGCCAAGAACAGTGCGTTCATACCCATGATGAGGCTAACCCATCACTCGGGAAATCTGAGGCAAAAGGATTGCTTCGAATTTTA
GGCTAGCCTAGGGTACATCTGGAGACCTGATATCTGAGTTAAGTTAATAGGAGCGAATGAGTTGGCATGGACAAAGTACCTGCTGTGAAAGCAT
AAGAGCTTTAACCCGGACCCCTAGCCAGGTCCCGTGGCATGCGACTCACCTGCAATCCTGAGGCTGGGGAGGGCGTACAGGTGGGTCCCTGGAG
CTCACAGATCAGCCGCTGCAGCCCAACCGGTAAGCTCTAGGTTTAGTGAGAGACCCTGCCTCAAAAAGTAAGGGGCTGAGGGATAGAGAAAGAT
ACCTGATATAGATGACTGTCCTCTACACATGAATTCACAGATGATCTCTCTCTCTCTCTCTCTCTCTCTCTCTCTCTCGCTCGCTCGCACGCGC
CACGCGCGCTTGTAAGGGACTGTGATGGACGAATGGGTGAAGCAGAAGGGAGCATGGGTGAGGCAGACAGGAAAGTGAGAAGTAAGCAGAGCCC
ATCACTGACTCTCTGACGCTGTGCTGATAGCTTGTCTCTTTGGAGGTGTGCTTGTCATTCTGGTACAGAGCCCCCCACCCCCCAGCCCTTTCCA
CACCAGCCTGGATGACTTCAGATGCCATTTCTATTTCATCCTTAGGAAGGTGAAGAAAGCTGGCCTCATGGTAAAGACCTGACATCCCAGTTAC
TCGAGGGATGGAGGCAGGAGGATTGCCAGTTCGGGAAACAGTGAGATTCTGTCTCAAAATAAAAGTCATAAGAGGGCTGGTGAGATGGCTCAGT
GGGTAAGAGCACCCGACTGCTCTTCCGAAGGTCCGAAGTTCAAATCCCAGCAACCACATGGTGGCTCACAACCATCCGTAACAAGATCTGACGC
CCTCTTCTGGAGTGTCTGAAGACAGCTACAGTGTACTTACATATAATAAATAAATAAATAAATCTTTAAAAAAAAAAAAGTCATAAGAGAGCTG
GAAGTATGGCTCAGTGGTAGAGCCCCTGCCTAGAATCCCCCAGGGAGGGGCTGGGGGTGTGGCTCAGTGGTAGAATATCTACTTAGCATATTGA
GGGTTTAAATTCAACTCCTATTATTTAGTACTACTAAGTAATATACAAATAAAACAAAGAAGGAGAAGCATCAGATCACGTTCCTGAGGAAGAG
CTTACAGGGAGAGACTCCATCATTCAGTAAGCAGAGTCCTATTTGAGACTCTGACTATCCAGGCCAGAGCCTCCTCCCACTGTCTCTGGGATCC
TATTCCACCGCCTTGGCATGGGGTCACGACCAGGAGGCTCCAGAACCACTGGGCTCACCCCTGCCTGCCTGCTGCAACTGCACAAGCTAGCCAT
TCTTGAAGAAACAAAGGCTGGCTGGGTTGTGGTGGCTCTGGCGTCTCCCTGGAGGGTGTCTGGCCTCTGCAGGATGTTGGGGAGTGGATCTGGG
TGGTGGTTGAACTCTGTCACTGGGTATCGCAAATCACATTAAGCCAGCCCTTCACCCCTCCATGCAGTCCAGCTGCCCTCTATGGTCATAGGGC
TTTCAGTCAGCTGCTTAGGCAGGGATGCTTTTGGCTCCCGGGCAGTTGAGTTCTCCCCATGCTGTGCAGACCCTTGAACTCCTGTGCAATACCT
GTGATACTGCTCCCTGCGAAGAGTTCGTGAAACCTAGTATTGGTCCCCAACTTCCTCCTTGAGATTTGTGTCCTGGGCCAGCTCAGAAGTCAGT
AGGTGTCCCTGACACACCTTCTTATTCCCAAGACCTTGTCCTTGGAGGACAAGGGGCAGCAGACTGAGTCCTTACCCCTTCGCATGTGTGCATG
TGTGCATGTGTTCATGTGTGCTCTGCCCCCTGACACCTGATACCTAACCATTACTCCTACAGTAGACACCAGGCTCTGCTTTAGGCATGGGCTC
AGCCTTGATGGTTTCTGCTCTCATAGCACTGACATGTGGCACCTTCACAGATATCCATATATCCTTTCTAGTGTCTAGCCATCCACTGTTTGTT
TGGTTGGTTTTGGTTTTTCAAGACAGGGTTTCTCTGTGTAGCCCTGGCTGTTCTGGAACTCACTCTGCAGACTAGGCTGGCCACGAACTCAGAA
ATTCACTTGCCTCTGCCTCTCAAGTGCTGGGATTAAAGGTGTGTGCCACCACTGCCCGGCTTCCATCTGTTGTTTACACATCCTCTTACCATTC
ATCTATCTATGCATCCATCCATGCATCTACCCATCTATGAAACCATCCATTTGACATCCCATATGTCCCTCCATCCTCTCACCCATCTACCGTA
CTGTTTACACAACCTCTAACTCATTCACCCATCCAAGCTTCCTTCCATGCATCTATCCATTTATCCATCCATTCATGTATCCATCCATGCATCC
ATCCAACATCCCATCTGTCCCCCCACCCACCCACATCATCTACACATCCTCCCATGCAGCAGACTACCCATCCATGCATTTGTCCATTATCCAC
TTTACCTACCCACCCATCCCATCCATCCACCTCCCACACATTCCATTCACACACCCCCACTCCATCCATTCCCCATCACCTAATTGCTCATTCA
TCTATTCACACATCCACTCACCCTCCCACCCATCTAGCCACCCCATTCACGCACCTACAGTCTGTCTGTCCATCCTCTACCCCCTGTGCAGCAT
GACCCTTGAACCCTGCTTTGTGGCAGATCTGGATGAGAAGTGAAATGTTACCCGCCTCCAGGGAGGGTGGGAGATGAACAGGCTGTTCTTGTTT
ACCAAGTAAGTAAGTAAGTAAATACCAAGAGGAGGGGTTACAAGGGCTGGGAAAGGGGAACCATTTAGGGCTTATCAGGATACCCCAAACCAGA
GTGCCCCCTGAAGTTTTGCCCAGCCACTTGGTCAAAGGAACCAGACTCTAACCCACACTGGGGTCCAGGATCAATCAAGCAACTGGGTCCATCG
AACTGTGCCTGCAAAGCGCCAGGACGGAAGGCGCCAGCGCCGTCCCTATTACAGAGTAGAGTGAAGCCCTCAGCCTACAGAGAGTCGGGAACTG
GCCCAGGACCACACAGAATGTGGCAGTGGTGAGGGGTACTGGGAGGAGCGGGCAGAGCGGGCAGATCGGTGGATCTGCATGGGGCGGCTGTCTC
TTGCAGCTGGAGTCTGTCGAATCTGTTCTCGGGTAGTGCCCACCCCAGTTGATGGGAATCAGCTGCTGTGTTAATTCTACCCTTGTATTCCTCC
ATGCTTTTGCCTCTCCCCTCCCCCTCTCCCCACTGGAGCTGGCTGTACTGAACTTCTAGCCAACCCCTTCCCTTCCCCCTTCTGCCAGGAGTGA
CAGACCTTCTGTACTGAGCCTCACCGGGCCATTTTGATTGAGCCCCGTCTCTGCTGTATGTGTACCAGATTTAAAAAAAAAAAAAAAAAAAAAA
AAAAAAATCCCAGCCCTACCTCATTGTACTGCCTTGCAGTGGCTTTAGGGATACATTTATCCTTGGGCATGTGTGTATGTGTATGCATGTGTGT
ATGTGTGTATGTATGTGTGTATGGCACACAGGTACACATGTGTGCCAGTAGAGACCAAAGGTCTCAACCTCAGATTTTGCTTTTGTTTGGTTTG
GTTTTGTTTTGAGACAGGGCCTCTCACTGGACTGAATCAACTCAACGGGTAAGCAAGGGTAGCTGCCAGTGAGCCCCGGGAAGCTGCTTGTCTC
TAACTCCTCAGTGCTACGATTATAAGCACACACCCATCATGCCTGGCCTTTTATGTGGCGGCTGGACATTGCACAGTAAACACTTTTGCCAACT
GAGGCTATGTCCCCTACTTTTGGTTTTTTTGAAACAGGATTTCATGTAGCTGAGGCTGGCCATGTTTTTCTGATTCTCCTGCCTCTGCCTTTTA
AGTGTTGGGATTACAGGTGTGCACCACCATGCCCAACCTCCCTGGCTTCCACTGCCCTGCTAAGGGAGGGCTGGTTCCCAGACTGCCAGCTCCA
AGAGGTGGTGGGTGTGGGCTGCTTTGGATGGCTTCCTGCTGAGTTAATAGTTCACGTATTTGGTCTCCATAGATCTTCCCAGGACCCTGCATGG
GAGGGCAGGCAACTGTCGGGGCCTCAAGACACCAGGAGGAGGGTGGTAGGCAGATGGCCAGTTCCAACAATTCCCAGGAGACTACAAGACTAGG
CCAGGGAAGAACCTGTGTCCTCAGGACTTCGTGAGTTTGACGTCAGCTGGAGAGCCCTGGCTCATTCTACAGGGGTACTCAGTTTCAACAGTCA
CTTGGGCATCTACCAACCAGGGACTGGCATAGTGAATCAGAGTGCAAAAACTACACCTTGAGTCTGAGAGCTGGCACCCTTACCTGCTCTGCCA
CAAAATGACAGGGGACCCAACTAGGTCCCCGATGTCAGTAAAAAAGGAAAAGTTAATTTTAGTGACTTGAAATGAAAGCTGTGACTCAAGTCAC
ATTAGGGGACATCAAACATACAAGATTCAAATCCTGCTCAGTGAGTGTGGGGGAAAGATTGTATATGGGTTCTGATTATAGAGAGGGAAATGAC
ATGGAGTTTCTCTGGAATCTATAATATGATGGTCACATCCTGGATAGTTTCTTGTTCTCTATGTCTCTCCCCTCTTTGTTATTTTATTTTATTT
AAAGAGTTTCTCTGGAATCTATAATATGATGGTCACATCCTGGATAGTTTCTTGTTCTCTATGTCTCTCCCCTCTTTGTTATTTTATTTTATTT
TATTTTATCTTATGTATCTAAGTACACACTGTGTAGGTTCAGATACACTGGATCCCATTACAGATGGTTGTGAGCCACCTGACCTGGAAGCTTC
TATATAGACCAGGCTGGCCTTGAACTCACAGAGATCTGCCTGCCTCTGCCTCCCAAGTGCTGGGATTAAAGGTGTACATGACCACACTAGGCCT
GATGCTCTCTCTCCCTTTAACATCATCCATGGCCCACTAGCCTTCTCTCCAGCTTCAAGGTCAGTAAAATCCCAGGACTGTAAGGAAAAGGAAT
GAAACTGAGGCCCAAGAACTCACAGCCTCAGAGAGGGTTTGGCTTAGCAGTAGAGCCCCTGCCTAGAATCCCTCAGTGAGGGGCTGGGGGCGTG
ACTCAGTGGTAGAACACCTGCCTAGAATCCCCCAATGAGGGGCTGGGGCGTGGCTCAGTGGTAGAACACCTGCCTAGAATCTCCCAATGAGGGG
CTGGGGGCGTGGCTCAGTGGTAGAGCCCCTGCCTAGAATCCCCCAGTGAGGGGCTGGGGGCGTGGCTCAGTGGTAGAGCCCCTGCCTAGAATCC
CCCAGGGAGGGGCTGGGGGCGTGGCTCAGTGGTAGAGCCCCTGCCTAGAATCCCCCAGTGAGGTGCTGGGGGCATGGCTCAGTGGTAGAGCCCC
TGCCTAGAATCCCCCAGGGAGGGGCTGGGGGCGTGGCTCAGTGGGAGAGCCCCTGCCTAGAATCCCCCAGTGAGGGGTTGGGGGCATGGCTCAG
TTTAGAGTGTTCACTGCCTGCACCCTAGTATTTCCCCTCCCAGAACTTTTTCTGAGGACCCTAGGGGCTTCCCGAATATCCTGATCTTCCCTCT
CTAGCTCCTCATTTTCTGTCCTGGGATGAGCGCCCTACCCTCCCATGCCCAGTGGGTGTCTGCAGGGAGTCCATGGGTCCAGAGGAAGGGAGTA
CCCCATGAGCTCAGTGCAGGAAGCAATAGGGAGAGGCGAGCTGGTTATGTCTCCCTCTGGTTTTCAGACCTCAAGGAGGAACCACGAAATTCCA
ATCTTCTGACAGCGAAGAAACTGAATCACTCTTGCCAGAGCCTGCAGCCCCCACAAGAATGCGGTAAGGCTTTGACCTGCCGTTCCCTGTCCAG
CCACCACAGTCAGAGCTGGTTGGTTCCTTACTCCATGGCCGACCCGGTCTCCCCTGCATGGCTCCTCTGTGGCAGGCATGGACACTGCACCAGC
CCCTCTCTGGGCATCAGGTTGCTTTCTTTACAATGGGGAACATGAGGACTTGGTGGTAGCGGCTTGACATGGTCCCAGGCTTGGGATGAAGGCG
CACCAAGACTGACAACCAGCTATGACTTGGTCAGGTGGGTATGGAAGGAAGGCATAGGTAGTTTGTTTCCTCTCCTGCCGTCAGAGACTTGATA
AATGCCCACGAAGGTATCTTCCCTTTGGTTTAAATGGAAGCCATGCAGAAAGTTAGGGTGATTAGGGGACTTGAGCTATTGGGAAATGAAGACA
TTGTATGTGCGGGGTGGGGGGACACGGGGTAGGACCACCACAAGACATGTGGATACCAGGAGAGTCTTGAATTCATAGGTGACAACAGAGAGTC
ATGCTATAGTCTATTCTGTGTCTGTGTTTGTGTGAGAGAGACAGAGAGAGAGACAGAGAGACAGAGAGACAGAGACAGAGAGACACAGAGAAAG
TAGAATGGAGGCTGCCAAGTGGTCCAGGAGAGGGGTCTCAAGGAGGAGCCTTAGACCTAAAACACTCGGAAGACACATAGATAGTTTTTGGTGG
GAATGGATTAGCCCCTGAGGCTGAGGCTGGAACTGGGGTGAGTTTATTTGGAGAGCAGTCCAGCAGATAGCAGGTAGGACTTTTTCCTTGTCCT
TGAGGGACCAGGCTCTGGCAGGAGGAGGAGGGGTTTATAGCAGGAGGTGACGCCATGGGAGCTTTGTCACTCGGGCACAGCCAGCACCATCTGC
TTAGGCATCTGCCTACCAGAGGTGGTGCCTCCTAATTACCTGGGGGGAGGGGTCAGGAAGAAGCCTGGGAACCACCATATCCCACTCCCTCCCA
CCACAGAGGGGTGCAGTGTCCCACAGGGCCCTCAGAGCAAATGGACCAGAGATCCACTCCTGGGTGGGGTGGGCTGGCTGGGGGGTGGGGTGGG
GTGGGGAGAGCCCTGGCTACACATCAATTCAAAGTCATTTTATTTTATATTTGTGATAGGGTCTCATGTAGCTCAGGCTGGACTCAAACTCACT
ATTTGCCAAGGAATATTTGGTGCAGAGATGAACCGAGGCGTCATACATGCTAGGCAAACACTTTACCAACTTTGCCAGATGAGGTACATTTCAG
GCGCTCTTTTACACGGCAGCAGGGGCTGACCCGATATTCACTCTGTAACTCAGGCTGCCTTTAAATGTTCAGCAATCGTCCAAGCCTGCTACAT
GGGCTATCATGTCTGTCTTCATTTTGAAAAGAATTTTGTTGAAAGCAGGTGTGCGTATGTGTATGCGTGCGTGCCTGCGTGCGTCTGTATATGT
GTGTTGCTCAGGCCAAGTTGCACGTGTGGAGGTCAGAGGGCAACATATTGTTGGTTCTTTCTGACCATGTGGGTCCTGGGAAATAAACTCAAAT
CTGCCTAGGTGACAGTTGCGTTTGCCCACTGAACCATCTGGCCAGCCCCTGCAGTAAGTCTTTACCTGATCCAGCCTGATGCCTTCTTTTAACC
TCCAAGAGAGGGACGGACATTAAGAAGAGTGTAGTAGAGAAATAATTAGAAATGATACTCCGGTCTAAAATATAAACGCTAGGGAGTCGGCGAC
AGAAGGAACAGCAGTTTATCCGTGGTGGCAGGGTGGGCTCAGACTTCCTTTGAAGGGGGACGCGGAAACTGAAGCCAACCAGACCCAGGGGCAG
CCAGGCGGAGAGCCGCCCCCAGCTGCGCTCCCGGAAGATGCAGCAGGCTCGGGCTCGGACCCTGCTCCCACTCCAGGGGACCCTTGGCCATTTC
CGGACCCGTAGCCTCTCACCCTACCATTAGGCGCACATTCCTGCCGCCCCCGCTCGTGCTAGGCGCAGGCAAGGCCCCCGCCCGTGGGAAGGGG
GCCGTGTGCCGGGGAGGCGGGTCTGGGATGGCGCACAGCTGGGTCACATCTGGTAGGAGTGCGGAGGAACCAAATGGAACAGAAGGTAGCCTTT
CATCCTTCGAGGAAAGAGGCTGCTTGAGTGTCTCCTCGCTACGGGGTGCCTGAGGTTTTTGGGGACCAACCCTCCCCATTTAGGAACATGATGC
CAAGACAGGCCTGCCTGGGGGAGGAGGGAAGGATGGACCGACGGAGGGACGTAGAATTTTGTCACTGATACCCTTTTGCCTGAACTTCAGGTCC
ACTCCTAAGACATAAGTATCAGTAAGGGCAGCTCAAAATTAATCGCTCAGGACACACTGCAGGCTACCACTTTGGCTCAGTCAGCACCGTTCCC
CAGGGAAGGAAACCCGCTCGGGGACAGGAAATGCACAGCGAGAAGCCAAGGATGTCGCCCGCGGAGGAGCACTGGCCCTATCACCCAGGTCCCA
GCTTCGGGGGCGCGTCCTCCACGTGCAGCCGTGGCGGGCGTTGGTCAGGCTGGGTGTCTCCCAGAGCAGCTCCGGAGGGTGACTCGGCGCAGGC
GGGACAGGCTCGTGCACAAAGGGGGCGGGTGCCGTGGGTACCCCAGCTGGGTGGTTCCCAGCCCCTTGTCCGTATCCCACGTGGGAAGGAGAGA
TCTCCCTAAAGGTTACGCCCCAAGGCTAGCAGCCAATTAGCGCGTGGAGGCGTGGCGGGCAGGGGAGGTTTAAGAGTTCCTGAAGCGAGAGGCG
CGCAGCTGGACCGGGAGCCTCGGGCAGCCGGACCAGTTGGCACTGGGATAGATATTACGTGCGGCCGCCGGCCACCATGCTCCAGCGGTGCGGC
CGGCGCCTGCTGCTGGCGCTGGTGGGCGCGCTGTTGGCTTGTCTCCTGGTGCTCACGGCCGACCCGCCACCGACTCCGATGCCCGCTGAGCGCG
GACGGCGCGCGCTGCGTAGCCTGGCGGGCTCCTCTGGAGGAGCTCCGGCTTCAGGGTCCAGGGCGGCTGTGGATCCCGGAGTCCTCACCCGCGA
GGTGCATAGCCTCTCCGAGTACTTCAGTCTACTCACCCGCGCGCGCAGAGACGCGGATCCACCGCCCGGGGTCGCTTCTCGCCAGGGCGACGGC
CATCCGCGTCCCCCCGCCGAAGTTCTGTCCCCTCGCGACGTCTTCATCGCCGTCAAGACCACCAGAAAGTTTCACCGCGCGCGGCTCGATCTGC
TGTTCGAGACCTGGATCTCGCGCCACAAGGAGATGGTGAGACCCAGCGCGGCCTGGGCAGGGGGATGAAGTGGGGATCACCCTCTGGTCCAACT
GGTGACTGACGATAGCGGTTCCCGATGCGTTAATTGCTTTGGGTCTATCTGGGAAACGATGACACTGATAAATCCCATTACAGCCCCGTGTGGC
TGCTGGGGTTCACTTTGCCTTACTACCCTGGAAGCTTCCTTCCGTGTTGCCCAGGGTCACCCCAGCCACATGCTCTGTGTCTGGATGAGTCGCC
TCCAGCTTAGGGCTGGCTCTGTAACGCTTCTCCTAAACACTGGAGTGAATGGCCATATTTGTTCCTAAACTTTTTCGAGCAAATTGGCTCAGTT
CCATCCTGGGTCCCCTTCACTCCCCTTCCTTCTTCCTGGGTGGAAGGTACGCCCCCTTGAAAGGGAGGGAGGAAAAGGCATAGCACACCCCTGG
ACCAGGCAAAGTGAAAGAAATGGGTGCTAGTTCTCCAGACTTGCTCAGAACCCTGACTTCAGTAGCTCAGCTCCCCCACCTCCTGTTAGACCAA
CTTCATACACATAATAAAAAGACACACCCCACTTTGAAAATCTCCCTGGAGGCTACAACAGGACCGTTGGGGCGGGAGAGCAGAGTGCACTGCG
TGTGTGTGTGTGTGTGTGTGTGTGTGTGTGTGTGTGTGTGTGTGTGTGTGTGTAGGGGTGGGGGTAGATATTTGGTGTGAAGACTAAGACTCAC
TTTCCTTCCTGACCCAGCCCCAGTGAGACTTCGCTTTAGGACAGTGGAGGTTAAGAGGACAGGCTCCTATCTAGTCTTGGTCAAAGAGGGGCCC
TGAGAACCCCACCGTAGCCTCCTGAGAATGAGGCGTCCCTTTCCCAGGGGCTCAGCAGGCCTTCGGGAGGGGGCAGCGGGGGGCCATGGGTTTT
GTTTGGCTGTGGTGCCGTTAGGATTCCCAGCGCTAGGCTGCTCTCACGCCGGCCCCTTGGGCAAGGCAACAAAGCCCCTTTCTCCAGCCGAGAA
AGGCGTCGGCCTCTTGTGTACAGCTATGCGCTCCATGGGAACCGTTGGCTGGCGTGCTGGCCACTCAGGTTGCCTGGTGATGAGGGGGCCTCTG
TGTCCTCCCCCCAGCCCTGAATGACAGCCCTCGTGGGGCACGATCCCTGAGCCAGGGCATTCCCCCACCCCTTCATTCAGCCTTCCTGCTGTGT
GATATTGGATAGCATCTATCCCACTCTGGGCCACTATCGGTTCTGAGAAACTCTTATTCAGGGATTTGGGACTCAGTTGGACTGTGTGGTCTGG
TGAGAAGCCTAGGGTCTCTCTGATCCTCTCTTGCTCTCAGAAAAGGGAAATACTTTCTAGTGAGGTATTATAAAGTGGACTGTAGAACTTTGGA
GGCTCTTGGTGATCCCAGCAGTCATTCTGAATAAGGGGGCCCCCTACCCAGAAAATGCTGCATTTACTGCCATGCTCACCTCCCGTGCACATGA
CAGGCAAAGCCCCCCTCTTGTGTGCTATGATCAGGGGCTCTTGGAACCTGTAGCAAAGGGCCTGCTCCCCTGTCTGTCCAGGGCGGCTGCAGTC
TGATGGAAGGTGAGAGAGGCTCCATTAACCTGGATTGGGGATGAATGGAAGGAAGGCTTCCTGGAGGAGGAGGTTCCATCTGCATCAGAGTCCT
AGGGTCCAGGAAGGGCAATCTTGGCACCAGGCATGACTGTGGGGCTGTACAAAAAAACCAAGCAGGCTCGGTGAGGTGGAGAGGCTGGTCAGGG
GTACAGGCAGAGCGCCTGGCCTCTGGCCCGCTGCTCCTCCCCCACCACTAAGGAGCGCGCCAGGCTGCAGGCTGCCACCGCCACAGCCACAATG
GCTGGGGCAGAACTTGGCATCCTGTGTTTCTTTGAATCTCGGTTCCAGGAACAATGCTGCTTCCTGTGTGTCTCTCCCCCACCCCCTTGCTTGC
CCTCACCAGCTCTGCTGCCCCCCTCTGCCCTGTCCGTCCTCCTTAGGGGAAGTTGGTGGGATGGGGACAGACTGAGATCAGGGTCTCCCATTAC
TTCTTCCTGAGTGTGGCCACAGGCCAGTGTCCTCTCTGGTCTCTTAACTATATGCCAAGGAGAAAAGAAGATGGTACTCACTTCCCAGGGACAC
TTGGGACAGGAGAGTCCATCCCTAGCACTTGGCTGTGCACAGGCAAGCTGTGTTGTGCCCATTTTACAGAGTTTCCGGTGGACTGAGTGATACC
CACAGGTACCCCTGCCCCCCTCCTCTCCTGTGGAGATACACCAGGCAGAATACCCACACACCCCACACTCAGCCTCTCAAAGACTGTTCTCTGG
GCCTCCCCCCCCCCCCACCCCCCAACTGAACAAAGGTGACTTTAATCTCCGTAGCCTTGGGCTGGGTTTTTAGGACACCATCGAAGCGCGGGAG
AACAGGCCTCTCTCTTGATCTCATTAAGAAAGCATTTTCCTTTTAATCCAGCAGCAGCGTCTGCACAAGCCGGCCCTCTGTACCTCCCACGGCA
TTCCTGGGCTGAGATGCTAGGGTGCGGGGGGGGGGGGGTGTGCTGGATGCAAGCGAGTCTGTGCGTGTGGGCTGCCCCGGGTGTCTGCAGAGAC
CTGGAAGGAGGGGGTGCATACTTGCAGATGATATAGATCCCACATTTCATCTCCTAACCTGGGCTTCACTGGAGTGAGCATCTGGGTATGGGAT
CAGCCTGGGTAAAGGAGTAAGGCTGCTCGAGGCATGGTGTGCCACCCTTGTAGACCTGGGGTTTCTTGGGTGGCTGGCTGGGTACATGCTGACC
TCCGTGTTTGCATGCTCACACCCAGGTTTCCTTGGGACCCTTTTGTGCACAAAAGCAGAGGGAAGGCTGGTGGATGTTGGGGAGCCTGCTGCCT
CTGCTGTGAGCAGCCCCCAACCGCCCTAGGGTTGCACTCTGCAATGCCAGGGTGCGGCAGTCGGCACGCGCTGGGCGGGCCGGCGGAGGAAGGA
ACATTCCTTCCCGTGTGCCGACGGGCCCAGGCGGGCGCCGGCAGCTCCTGGCCCCGCCCCGGCGCCCCCCTCCTCTTCCGCAGTGCAATCCTGG
CAGGGGGTGGCCCAGCGCTGGGCCAGGTGGAGTCTTGGCTGTGGCCCTGGGCAAAGCCTTCTGTAAGCGGGGAACAGTTTTCCTCTCTGAAGCC
TCACCCGGCTGCTCAGATACCCCAGTGGGCCTGGAGACATGAATCCTTTTCTGCTACTACCATGTTGACCGCCGTGACCTCCCAGAACCTGACT
GGGTGCTATGGCTGGGGAAACCTCTTCTATGGGGCACAGGAGGAAGTCCAGGCCAGAGTCGCGAGCGACCAATGGCCTTACCCCATACTCCCGC
GCACAGGACCTCTTGCCCAGCCGGCCCAGTTAACCAGGCCAGCTGTATGGAAATGACAGCCCGGCCACTCCTGCTTAACTCGGCAGAGTTAAGG
CTGGCCCAGCCCAGGACAAAGCTGGCGGACAATGGCTGGTGATTCATCCTCCCTTCGGGTCCCTTTGTCTGCCAGGGCTTGGCAGCGGCCCAGC
AGAGGCCTTTGTCCCCGACCCGAGTGAGTGAGGCTCAGCTTCCCAAAGCCGACCTCCTCCTCCTGTCCCCCGCCCCCTCCCACAGCTTGGAAAG
CCTGCAGGCTGGGCGGGCTGGCAGGCTGTGTCAATAAGGGTGTGTGCCCCGCCAGGTGCACAGGTTGGCGGAGGGTTAGTTGAAGCCCGTTTAC
CTGGCTGGTGGGGGGGGCATCCCACCTGGCTCGTGCCTCCCAGCTGCCTGTGGTGACCTTGGGCCCACCTGTCTGAAGTTGGAGAGTGAGCCCA
CCTCCCCCAGCCAGCCAGCCCGCCAGTCCAGCTGCCACTCCCCCTGGGCACTTGAGCACACATTCTGTGTGCAGTGGAGAATTCAGGCAGTTCA
GTGCAGGGTGGGGGAGGAGTCTAGATTGGGTGCCTTGGCTGTGGTCCTCGTAGGGCACTTTGGAGCTGGAAGGCTCAAGATCCTGTCTCTTCTT
CCAGACGTTCATCTTCACTGATGGGGAGGACGAAGCTCTGGCCAAGCTCACAGGTTGGTCCCAGGCTTGGGGGTGGGGATGAGGGTGGGAGGCG
CTGGTCCCAAGCAACTTCTTCCCCCGAGGCTGTGAGGTCACTGGCTGCCGCCTCGGGGCTAAGAGGCCAGTTCACTCGGCTTGTTTGCTTCATG
CCCCGCCCCACCACTCCCCGGGCCCCGATTCTTATGCAGAGGAGACCCTTTCAGTCCTCTGGGGACCCTTTGAGTCCCGCTTAGCAGCAACAGG
TGGCGGGTACTGGGGGAAAAAAAACTGGCTGAATGAATGGGCAGCGGTGGCTACCCTGGGGAGGGGGCTAGGCTGGGCAGGGCTGGGGCTAAGG
TTCGAAGCAATGAGAAATGCCTGCCGCTTGTGCGGACCAGGCCTGCTTACGGGCCCTCCCCGTCCACAGGCAATGTGGTGCTCACCAACTGCTC
CTCGGCCCACAGCCGCCAGGCTCTGTCCTGCAAGATGGCTGTGGAGTATGACCGATTCATTGAGTCTGGGAAGAAGTGAGTTCCTACCTTTCCC
TCTGTGCCCCATCCGTGCCCCTTCCCTGGCAGCCCGGCAGCCCTCGACCCCTGTCTGCAGCCCCTGACAGCATCCCTCCCGCCTTGTGTTCAGG
TGGTTCTGCCACGTGGATGATGACAACTACGTCAACCTCCGGGCGCTGCTGCGGCTCCTGGCCAGCTATCCCCACACCCAAGACGTGTACATCG
GCAAGCCCAGCCTGGACAGGCCCATCCAGGCCACAGAACGGATCAGCGAGCACAAAGTGGTGAGTGTCTCCCAGGGTAGCACACACCCTGAGGT
GTTAGGGAGGGGCTGAGCCACCTTTGCAACCAGGAGTCTCCTGACAGCCCTTGTTTCACTTCACCCAGAGACCTGTCCACTTTTGGTTTGCCAC
CGGAGGAGCTGGCTTCTGCATCAGCCGAGGGCTGGCCCTAAAGATGGGCCCATGGGCCAGGTGAGTGTCCCCTGCCTAGTTGCCACTACCCCTG
ACAGCAGACTGTTCCTGGTGCTCGCTCTTGGTCCTGGGTTCCCTGCAGCAGCAGCGTCTGGCTTGGGCTATTCTCTCCCTGCCTTAGGTAGCTG
TGTTTTCTGCCACTCTCTCTACCTCTGATAAAGTCTTCGGCATGGAAGGACTCTGGCTCATGGCGGGGCGGGGCATAGTGGACTCCCTCTTTTT
GGGGCCAGTTTGGCAAAGTCTTGTCTACTCATGGCCCAGCTACTGGCTTCAGCCTCTACTTACTCTTCCCCGGGCCCTTCCCTTGCAGTGGAGG
ACACTTCATGAGCACGGCAGAGCGCATCCGGCTCCCCGATGACTGCACCATTGGCTACATTGTAGAGGCTCTGCTGGGTGTACCCCTCATCCGG
AGCGGCCTCTTCCACTCCCACCTAGAGAACCTGCAGCAGGTGCCCACCACCGAGCTTCATGAGCAGGTGCGCATGTGGCCCCCAGGCCTGGGTG
ACCAAGCAGAGAACTCAACAGTTGAGGGACAGAAGGGAGAGGGTGCCTCCAAGGAAGGGACAATTTTTATCTATTTATTTATTGATTGATTGAT
TTTGGTTTTCTGAGACAGGGTTTCTCTGTGTAGCTCTGGTTGTCCAGAACTACCTTTGTAGACCAGGCTCGCCTCGAACTCAGGGATCAACCTG
CCTCTGCCTCTGAGAGCTGGATTATAGGCATACTGATACAGGGCTTCACTATGTAGCCCAGGCTAGCCTTGATCCTCTTGCTGTAGCCCCCCAT
ATGCTAGGAATCAGCTGTGTGTCACCACACCTGATTCAGAGTCGTTTAGGGGTGGGGTGTATGTATGTTTTCATTTATTTATTTTTTTATTTAC
TTAGAGATGGGTCTCTTATAGCCCAGGCTAGCCCTGAAGTTGATATGTAGTTGAGAATGACCTTGAATTCCTGATCCTCCTGACTCCACTTTCC
TGGGCTAGGGTTGTAGGAGTGTACTACCACATCCAGTTTATGCAGTTAGTTGGGCTAGCCCAGGGTCACTGTTTTAAATAGGGATATCAGAGAC
TCCTCATTCTCAGGTGTCACAGCCTGCTGGTCCACATGAGGTACCCCAGTTTCACTCTCTAGCTCCTCCAAAACAGAAAGTCAAATAGATTCTT
CTAAGTTGCTGATCATGCCCTTTGAGGGGACACTCTTGCTCTGTCTCCCTTATGGTCGTAGGCCCCACCATGGGCTTCATCCCCAGGTGGGCCG
AGCCAGTGTCCTTGGCTCAAGGGACAGCAGCAGGAATAGCCAGGCAGGACTGCCACCTCTCCAGCATCATCCTGTTGCTCCCAATGCAGGTGAC
CCTGAGCTATGGCATGTTTGAGAACAAGCGGAACGCAGTGCACATCAAGGGACCATTCTCTGTGGAAGCTGACCCATCCAGGTAAGAACTCGTA
GAGGGTCACCAAGTACCCCCGGGGTTGGGGGGGTGGCAGGAACCCCATGGTTGGGACTGCCAGAGGGCCCATGGCACCCCCAGCCCACGCAGAC
CAGTCTTTACTCTGTCCCAACAGGTTCCGCTCTGTCCATTGCCACCTGTACCCAGACACACCCTGGTGTCCTCGCTCCGCCATCTTCTAGCAGT
CGTGGTTGAAACTCTGTCCCTGGGCGCCCCTGGTATCCAAAGGGCCCAGGGACCTTTATTGTATGCCCTGGCCTTGGTGTCCGAGGCTCTTCAG
GGCTATGTGTATGTGTGTGTATGTCTATATTTGTGTGTTTGTGTGGCGTGCCCACCCGTGTTGCAGACTGCTGGGCAGTCGTGTTCTTCAGGGA
GTGGCAGGTCTGTCCCTAACTACACTCTGAGCACCACTCCGAGGGCAGTTGTGTGGGATCTGTGTTTGGATCTCTGCAGTGGGGGTGGGTGAGG
CCTTGGGGCTCCACTCAGGGGTCCGTGGGTGCTGATTGTAGCAGTCTCTTATGTTGGGGTGCTAGCACCTATCTGGAGCCTTCCCTGTCAGCTG
AGCTGTGCCCAGTCCTAGGGAAGCTGATTTGGGTAGTAAAGGCCTGGACCCCTTGTGAGCTCTGGCCCGCGCGCTGCCTGGTGCCATTCTGGTG
AACAATGCTCCCCCACCCCCTGCCCTCCAGGGTGGAGGCGAAGTCTTCAGCCCCACCCTCTGATGCCCTATCTTTGTCTCCCACACCAAGTGGG
GTTCAGAGCTATGAATTTTATCTCCTCTCCAAAGTAGAGAGAAAGTCGCCCATAGTGGGTGTGCCTGTACATATTGTGACAGTATTTTTTTACT
GTGCTCTTTCTTGAAAGGGTCACCCCCACCACCCCCAACTCCTCACTCCCCTACCCCTGGGCTGTATTCTGTGTTCTTTTTGCAAAGACCTTAA
CTAGGCAAGCTAATGATGATAAGGGAAAAGCTCTCAGGGAATTGATGTGTTGTTGCTATGGTGACGTCCTTCCTCTGAATAAAGGTGCTCTTTG
CAGCAACCCAAGGGGCCCTAGTCTTGCTTGGGAGTTGGGGCTAAGCCACCTGTCCATCAAGGTAACTCGAGGGGACTTTCAGATGAGGGCAGGG
GAGACCCAGTCATGACATAGAGAAGAAGGCAATTGGTAAGAATTCAGCAAGTTAGCCGGGCATGGTGGCGCACGCCTTTAATCCCAGCACTCGG
GAGGCAGAGGCAGGGGGATTTCTGAGTTCGAGGCCAGCCTGGTTTACAGAGTGAGTTCCAGGACAGCCAGGACTATATAGAGAAACCCTGTCTC
TAGTGTTAAAATAGATGAAGTATGGCCAGCTTGGTGTAGATGTGCCTCTGATGGGTTGATACCATGTTGGGAGGTGGGAATTGGACAGGGCTGA
TGGCGGTAGCTGCTTGATCAGGTCATGCCCTGTGTCAACCTCACTGTGTCCAGACCTTTCTGGAGGAGGTGCAGTGGCTAGTGGGGGCTGGCCA
GAGACCCATTTTCCAGACAGGAAAACTGAGGCTCAAAGCAGGCTAAATGAGGATGACCAAAGTCAGGCAGCCAGGCTGCTGAGGTGGATGACCC
CCACCCCCCCTAGATCTTCGTTCCCTGTCAACTCTGCTGACACTGCCTGGCCTCTCATCTGTCATGAGATACACCTGTCATGTATCCTCTGAGG
ATGGGCAGTACCCAGGCTGCTCACTCGTGGGTAGAGCTGGCGAGGACTCGGATCTTTCTGAGGCTCTTCAGGGACCTTCCTCAAATCCAGCCAC
AGAGGCTGGCTGATCGGGGGATCCTCACCACACTCCTTCCCTGTGGTCTCCTTGTTTCCGCTCCTTTACAGAGAAGCAGGCTAGTGCTGCCCAC
CTGCTGCCCTTCTGTAGGTGGCTGCCTGCTCTGACCTGACCCCGGACTCACGGGGCTAGGTCCTCATTACCTGCCACCCCTGTGTCCTTTGCCT
TCTTCCCCACACCATTTGATTAGAGATCTGTCCTGCACTCCACCAGACATTGCTCACAGCTGTGGGTTCAGCACCCCCAAGTGGGCTGGGCATG
GCCCTTGCTGGTAACTGCTCTTGCCTTGTGGGGTGACTGGCCAGAGCCTGACCCCACCTTGGCTTTGTAGTTTGGCTTTTTAAGGCCTGAGGCA
GCTGCCTGGGATTTTCAGAGTAGGGGATCCCCAGTGGTGGCCGTGGTCCTTGGCATACAGGGCCTGGTGGCGGTGGCTTGGGGCCCTACTTCTA
GATGAGGAAGCTGACATTTGTAAGGGTGGAAAGGTCACGGTGCAGCAGTCTGCACACGGGCCATGAGCCTCCTTCCTGCCTGGATGAGTCAGAG
GCCTGCTACCCTCTGCTCTGGTCGTCAAAAGCCCCTTGCAGCTGGCACAGAGCTGGCAGCCACAGAGGTGTGGTACAGCGGCACCAACCTCGCC
GCCGGCATGGAGCTGGGGGCCCCAGCCAGGGTGAGCTTCCCATTCTGATTGGAGCCAGAGGGTGGTCCTGAGCCTGGAGGGCAGAACCTCAGCA
CGGAGCAGGAGGGGAACCCAGTGAGGTGGCATAGGTCTCTAACCTAGTATTATTCATGAGGCTGTTGAGGTAGGAGGATTGTGAGGGTCGTGAG
ATCAGAGCCAGCATGGGTTACCAAGTGAGGTCAAAAAAATCATCTAAGAAACAGAAGCCAAGTAGGGAGGGCCCAGGAAGGGTCTAGCCAGTGG
CTTCAACAACTCCCAGGTAGAGCAGGCGGAAGAGTTTCTCCTGAGGAGGGAAGCAAGCTGACTGACTGACTGACTGACTGACTGACTGACTGAC
TGATTGATTAATTGATTTTTTTTCAAGACAGAATTTCTCTGTGTAGGCCTGCCTGGCTGTCCTGGTTGATCTGGAACTCAGATTCACCTGCCTC
TGCCTCCCAAGTGCCGGGATTAAAGGCGTGCGCCACCAATGACTGGCTGCCTGGCCGCCTCAGTGAGGGTCTTAGCGAGGAGCCCTCCTCTCTG
GTCAGTTACTCAGTCAGGTATCCTGTCCTGGGGCTTCCAAAAGTTTATAAAACGCCAGGTACACTGATGCCTCGTGCCTGTAAATCTCAGGGAT
GGGGAGGCAGAGGCAGGAGGATCGCCAAGAGTTCAAGGCCAGCCTAGGCTACCTACTGAATGATGCTCCTCCCCTCCCAGCTCTCAATAAAAGT
AACACTGCACACCACGCAAGCTCTAGGGTGTCAGGGAGGGTGCAGAAGCAGGAAGGCACCCAGCTTCCTCTCTCGAGATGAGCTGGGTCTGGGA
GAGGGTGGCAGGGTAGGGTTGAAGCTTTCAACTTCCCTCTCTGAACTTCCCTCCCCTCTGGGGTCAGCAGGGCTGACTTCCTGGAAGAGGTGCC
TCACTGCTGGAGCCGGAAGGCCAAAACCACAGCGTGTGAGCAGAAGTCAGCATGTGTTTGTGGGTGTTCAAGCTTGGCACAGCCCAGACAAATG
CACCCCAGGGTGGGGTTTGGAGGGACCTGGTGAGGAGAAGGCTGGGAAGGGCAGTGAGCAAAGCCATTTCGTCTCTCCCTGAGGCTGGACCTCT
GATGGTAGAAGTCAGAGCCCGCTGGGTGGCTTCAGCCTCTGTTCTGGGCCTCATGAGGTCTCAAGAGGAAGCAGAAGGCCCTGGGCAGACAGCA
ATTTGTCAGACCCATTGGCCTAGCCTGTGGCACATTCTCCTAGGGCCACCTGACCCCAGCCTTGACCTCTCCCCCACCAAAACAACAAAAAAAA
TTTATCTTTTCCTATTCATGCTCGGCATTTAATGTTTTTAAATTTTTTTTTTTAAGTTGGGGCTGGAGAGATGGTTCCATGATTGTTGCTCTTG
CACAGCAAGGACCCAGGTTCGATTCCCAGCACCCACAGGGCTGCCCATAACTGTCTCCAATCCTAGGGGATGTAACACCATCCTCTGGCCTCTA
CAGACACCAAGCGTGAATGGTGTACATACAGACAAACGCCTATGCATGTAACTTATAAAAAAAGGTTAAAAATATTGGGGGTTGGCATTTGTAA
ACATTTTTTAAAAAGATATATAAGTACACTGTACCTGACTTCAGACACACCAGAAGAGGGCGTCAGATCTCATTACAGGTGGTTGTGAGCCACC
ATGTGGTTGCTGGGAGTTAAACTCAGGACCTCTGGAAGAGCAGTCAGTGCTCTTAACCTCTGAGCCATCTCTCCAACCTTGAGAAATGGTTTTT
GTGTCTCATGTGCAAGTTAGGTCACTAGACTTAACAATACGTACCTTAGCCATCTGCGTCATCTCAGTGGTCTTTTGGCAATTTTTTTTTATTG
GTGTATTTCCACTGTACACAGTAATTTCATTAAGATAGCTCCATTTGTCACATGCTTTGACCTATTCATCCCCTCTACCTCTGTCCCCTCTAGG
ATAGTCGTCTTTCTGAGATAGCCCACGTGTACTTTCATATCACACACATACACACACACACACACACACACACACACACACATCCATGAGAACC
GACAATATGGCTCAGTCAGTAGAGTGCTTACTAAGCATGAACCAAGTCCTGGGTTCAAATGAGTATCACATGAACATCTTTGGATACATAGAAT
CCAAGGCCAGCCTGGACTCCATCGTGAAAAATAAGTAAGACTGGAGAGAGAGCTTCAGTGGTTAAGAGCACTGGCTGTTCTTCCTGAGGATCTG
AGGTTCGAATCCCAGTGGCTTAGAACTGGCCCGATGCCCTCTTCTGGCCTCTGCCGGTATTGCATGCATGTGATGCAAAGACATACATGCAGGC
AAAACACTCATACACATAAAATATTAAAATAAGTAAAATAAGAGGGGGGGAATGTATTAAAACTAACCAGCCTTGCTTTCTGTGCAATTACATA
TGTAAATCCATGGAAGGGACCCTAGGACCAAGAGGCACCCCATATTGAAGGTTGCTTAGGTATTGTCAAGAGTTTAGAGGTCCCATCAAGGCAT
CATCCACTTCCACCACAACCCACCATGGTCCTTGGCTGCCGCCTGGTGGCCACAGTTGATAACTTCCCACCCTGGGACTCCCAGTGTGAGTGCC
AGCTTTCCAGAGACCCTCCCCCAGATCTAGGGCTACAACCAGGAAGAACTGCAGGCCTTTGCTTTGGCGTAAGTTCCATGTGATACTAAATATA
TTCCCTTCTGATTTATTGGTCTTTCTCCCTTAGTAGTGCTGAAAATAGAGCCATGGGTCTCGCATATGTTAGGTGAATGCTCAAAGTCCTGAAT
ACATCCCTGGCCTTCTAATGTCTGATTTATGCAATAAGGGAGTCAAGTAGAAGGCTCATGTGCTGTCTCGGGCTGCCTATCAGTGTGGCTCAAG
CCGCAGGTGCCAGCACAAGCATTCTCAGCAGAACTACCATCCACTTTCCTCCTCAACATAAACCTCTGGGCATTGTGGATTCAGACTGGCAAGT
ACTCTCCTGATGGGGGGACCAGTGCCTCCTGCTGGACCCTCGCCTCCCTTGAACCTTGTTTCAGCTTTCACATACAGCTCCCTCACTGGGGGCC
TCTAGGTAGGACCACTGAGCCACACTCTAGCCCATCACTGGGGGATTCTAGGCAGGGGCTCTCCCACTTAGCCACGCCTCCAGAGCCTCCCTGG
GGAATTCTAGGCAGGGCTCTACCACTCAGGGGCTCTACCACTGAGCCACAGCCCCAGCCCCTCCCTGGAGCCATGCTTTAGTACAGAGCTTTCT
GATTGGTCCTTGGCTTCTATACGTCCTTCATCCCTTCCTGACTCAGCAGCTAGAGCAATTCAGTCGAAATGAATACTAGATAGTGTCACTCTAC
GGCTCCTTCCCAGCCAATTGCTCCCACAGACCTCATTCAGCAACCCTGTGTCCCTAGCCTGAGCTACTCTGTGTACAGTGTTCATGATGAGAAT
GAGAGAGAGAGAGAGAGAGAGAGAGAGAGAGAGAGAGAGAGAGAGAGAGAGAGAGAGCTGTTTCTTAAAATTGTTTATTTTAAAACATGAAACC
CAAGCTGAATAGAGACCACAGAAATTACATGGACAAGAACTTTAGTTATTTGCCAAGAAAAATAAAGTTCCCCACAGAAATAAAAATCAAACCC
CAAACCCAGTTTAAAAAATGAAAGCACAGCAACAGTAACACACAGCCATGGCCCTACACAGGCTGGAGGGGAACTTGGGGCTGGCTCGATTCAG
ATGACATAGTCCACTGGGGGCAGATGCAGGCAGAGGGACCGTAACTGGGAGGCAATCTCACAAAATCACATATGGGAGGAAGGGTGCGGCACAC
CACCGCTGTGATGAAAAACAAAGAGACCCTGGTCACCTGTGGCCCACCAGCTAGCCACCTTCTGGGCCGGACAGAGCTGGGACCGGGTAGGGCA
AGCTTTGGAGCTGGGGTCAAGAATTGCATAGGCTTGAAGGATGCTCCAGGCCGGGATCTTCTGCTACTCACATAAACCAGAGAGTGTTGGTTGG
GGTGGGGGCCTCCAGTCCTATCAGTTTTCTCAGCCAGATCCCTCCTCCTGGCTCGGCAAGGGATCGGGCTCTGGTTTGGTGGCTTAGCCCTCCT
TGTTACCCACCAGAGCTGTAGGGTCCCCTTGGGTAATAGGGACTGGGGACCAATCACAGGCAGCTGGCACTGGACTGCTCACTCCCTGGGGATG
TGGGGGTGGACCACTGGGATTGAGTAGGTAAGTGGCACCCCAGAAATCAGGAAAACGAAATGAAGCCAACCGCAGTGGTAGGAGCCGTCTCCTC
CGCAGGGGTCAGGGCGAGAAACCCCTGCTTTCATTTCCCCTGCTTCCTTTCTTCAGCCTCGGTGAGTGAAACGTCACAGATGGGGGCACTCACG
CACAGAAGGTCAGGACTCCCGAGACAGCACAAGTTCACAGCCACGTCCGGGCAGTGCCCGCCTCTCATGCTATATACATTCACCTTGTGGCCTG
CTCACGGCCAGGGGGCAGCATTTGTGCTTCTGAGAGGCCCCAGGGCACACCCAGGCACCCAGGCAGGGATACCCCAAACCTCCCTCCGGACCTT
AGGCCAGGAACATACCCATAGTGGGCAGGCTGGCTGCAGCTGGGCTGCTCTGGCCCTGGAGGTTCCTGGCGCCAGGCCAGTTGGAGTCAAGGCC
AGGGTCCCCTGCACTGATGTTGTCGTGCTTAGGAGCTACAGATGCCCCGACTGGGACCCCTAGGCCCTCCGAGTCCTCTGCCCACTCCTTTTGG
TCCCAGACACCAGCACAAAGAAAGGGATTTTGGGGAAGGAAAGCAACGTGGAGCCTCTCCCCTCTGGGGGAGCTGAAGCCCAGGGGGTTCTGGG
ATCCGTAGCTGCCAGGTTGCTCCAGGAGGGCCACAGGTCTGTAAACCACTGGGTCCCAGCCTTGCCCCTCCCACCACATGCTGCTCCACTGGCC
CGTAGCTGCCCATCTCACAGACCAGAGCTCACACTGGCTGGCTGGGCCAGGGTAGAGGCTCCAGGTCCTGATTTGGAAGCTAATTCAGCCCAGA
AGCACAGCAAACATCACACACTAAAGCCAAAAGCACCTTAGGCAAGCGGTGTTCTGGGTGTGGGGGGCTGAGGCCACATGGACTTCAGTCCCCG
GACCCCAGGACATGCTCCTCCTGGGGCAGCACCTGCCTGGGGAGGGGTCTCTAGGCCCTTTGAGTAGCCTGTGGGTCTGGCCCCTCCCTGGCAG
CACCCCACAAAGAAATGACCACTCTAGCCCTGGCTCCAGGCTAGACTTAGGGTCACGCTTGCTGGCTCAGGGTCAGCCAGGAGTGCCCATGGCA
GCTGAGAGTGAGGTTAGTGGTTAGTCCCAGCGGGGGGTGGGGGGGGCCTTCTGAGGCTGGGGGCACATTCACAGCCGTCACAGGTCCAGCCCCC
AAGGCTGGGCTCCAGAGGGCACTGCCGCTTGTCCTGGGTGAGAATGAAACCGAGAGGAAAGTGGGCAGTGTCCTGCGTCGCCTATGTCCCAGAC
TGCCCTGGAGATGACAAGTGTGGTCAGTGCAAAAACAAAGGGGAAGACAAAGCTGCCACTCTTCAGGAATGAATGGGAAAGAGGGTTTGGCACC
AAGAAGGTGGCCAGCACTGAGAGGCCAGGGAGTGACCTGGGCTCAGCTTGGCACCTGCTGTCTGGGGAGGGCTGGCTGGGCACTGAGGGAAGAG
TCATGTGGGGAGGAGGTGTAAGAAGAGCCCCACCCTAGAGGCAGCTCAGAACCAACAGGAAGAGCCAAGTAAGCCAGGTGGCTAAGGGAAGGCA
GGCGCCGGACGTTGCAAAGACCAGAGAGTATGAAGGGGCCGTTCGGTGATGAGCAGGTGGGCGCCAGGCACAGGGTCCCCTGCCTGTGAATGGA
GATGGGATTCTCAGGAGTGTGGCGGTAGGGCAGTACCAGGAAAGGCTCGCTGTAGCTGTGGGGAGGGGGACGCCCAGAGTGCAAGGGGCAGCAT
GAAGGGTCACCTGTAGAAGGAACGCAGGCAGGGTGGAAGCACCCGGCACAGAGCAGGGCTCAGGGAGCCCAAGAGGACAGAAGGGCAGGGGAGA
CGGAGGCAGGCAGGGTCAGGTTGCTGGTGGCAGCGACTGTCCAGGTGAGCACCCCAGAAGCCTCACTTCACAGGCTGGCCCGGAGCTCATGCTG
GGCACCTCCATGCCTCCTGCAGGGTGGGTATAGCGGGGGGGCCTTCAGGTGTGCCTAGGGCTGGCCCGGTGTGGCTTGTGGAAGGTCCAGCAGG
GCGGAAGCAGCGGTGCTGGCATAGGAAGGGTGGGGCATTGGCACGTGGGGGCAGATGGCTGGCCGGTGTGCGGTCTAGTGCCCGCTGCCGCTGG
AGTCGGGGCGAGGCTGGCTCGTGGCAGAGGTATTTGGCTCTATGCTGGAGGTGTACTAGAGGAGAGAGGGAAGGAGGGAGCAGGGTCAGGTGAG
TTCCTAGGTGGGGGCTGAGCAGCCAGCAGCCTCTTTGATGTGGTCCTTGGTAGGGTGAGCCACAGCCGACCCTTGGGGAGAGCATGGTTGGCGG
AATCCTAAGGTAAGAGGCCACTGGCCCAGGGCTGGACCAAGGGCTGAGTACAGGTTAGGGAAGTGATGGTACCTAGAGTACAAAGGTCAGCCTC
TGTAGCTGAGGCTGGATCACCCAGGCAAGTTCCCTTCCCAGTGAAGGACACCCAGTCCCCTGCTGTCGCTAAATCACTTTCAGAGTCCCAGTCA
GATCTTTGCCACCTCCAGTAATTCAGCTCAAAGGCTCTGAATGCATCACACACGCTGAGCACAGGAAGGCCACATGGACCCCATCTCTATACCT
CTGCCTCAGAGCTGTGCTGGGGTCAGGGCAAGGGCTCCCAGGGCAGGCCCACAGGAAGCTAGGCCACAGACCACAACTGCTGAGTCCACTGGAG
CCTCCTCCCCTGCCATTCGACTCCATGACCCCCAGGAAATGGGGGCCCTTCAGGCTAGAGTGACTCTGGCCCAGCCCTTGGATGGGAAAACTAA
CTAAAGGATGTGTGACTCGGGATAGAGGTGACTGCCCCATTCTGCAGGTGGCCTATACACCTTGGCCGCCTTAACAACAGCACCACAGGCTCCA
AGGTTTGGGCAGTGTGTCCACGTGCACGCATATGCACAAGCACAAGTGTGTGTGTGTGTGTGTGTGTGTGTGTGTGTGTGTGTGTGTGTGTGTG
GCTGTCTGCCTGCTTGCCTGCCTGCCTGCAGAGGAACATGCCTCCAATCATGTGCCAAGGCCAGGTAGTGAGTGTGCAAGAGTTTGCATGCCTG
AGGCAGGGACAAAGGTCACCAGCACTCAGAAGACCAGGCAGGTGGATCCCCACTCACCCCACCCCAGGGAGCACAGGCTGGCTCCATAGTTCCC
CGATGAGAGACAATGCTTTCTCCCAGGGTCCTGAGTGCTTTCCTGAGACATTCTAGGTTAAGGTTGCAAGCGGCCTATGAGGCGGCCATGGTGC
AGAGGTGGGAAGAGCAGGGGCCCAGAGCCATGTCATTCCATGGACATGCAGAGGTGATAAGGAACAGGGGGTTGGGATGGGACAACTGGTGGGA
TGGGACG
MOUSE SEQUENCE - mRNA
CTGGCACTGGGATAGATATTACGTGCGGCCGCCGGCCACCATGCTCCAGCGGTGCGGCCGGCGCCTGCTGCTGGCGCTGGTGGGCGCGCTGTTG
GCTTGTCTCCTGGTGCTCACGGCCGACCCGCCACCGACTCCGATGCCCGCTGAGCGCGGACGGCGCGCGCTGCGTAGCCTGGCGGGCTCCTCTG
GAGGAGCTCCGGCTTCAGGGTCCAGGGCGGCTGTGGATCCCGGAGTCCTCACCCGCGAGGTGCATAGCCTCTCCGAGTACTTCAGTCTACTCAC
CCGCGCGCGCAGAGACGCGGATCCACCGCCCGGGGTCGCTTCTCGCCAGGGCGACGGCCATCCGCGTCCCCCCGCCGAAGTTCTGTCCCCTCGC
GACGTCTTCATCGCCGTCAAGACCACCAGAAAGTTTCACCGCGCGCGGCTCGATCTGCTGTTCGAGACCTGGATCTCGCGCCACAAGGAGATGA
CGTTCATCTTCACTGATGGGGAGGACGAAGCTCTGGCCAAGCTCACAGGCAATGTGGTGCTCACCAACTGCTCCTCGGCCCACAGCCGCCAAGC
TCTGTCCTGCAAGATGGCTGTGGAGTATGACCGATTCATTGAGTCTGGGAAGAAGTGGTTCTGCCACGTGGATGATGACAACTACGTCAACCTC
CGGGCGCTGCTGCGGCTCCTGGCCAGCTATCCCCACACCCAAGACGTGTACATCGGCAAGCCCAGCCTGGACAGGCCCATCCAGGCCACAGAAC
GGATCAGCGAGCACAAAGTGAGACCTGTCCACTTTTGGTTTGCCACCGGAGGAGCTGGCTTCTGCATCAGCCGAGGGCTGGCCCTAAAGATGGG
CCCATGGGCCAGTGGAGGACACTTCATGAGCACGGCAGAGCGCATCCGGCTCCCCGATGACTGCACCATTGGCTACATTGTAGAGGCTCTGCTG
GGTGTACCCCTCATCCGGAGCGGCCTCTTCCACTCCCACCTAGAGAACCTGCAGCAGGTGCCCACCACCGAGCTTCATGAGCAGGTGACCCTGA
GCTATGGCATGTTTGAGAACAAGCGGAACGCAGTGCACATCAAGGGACCATTCTCTGTGGAAGCTGACCCATCCAGGTTCCGCTCTGTCCATTG
CCACCTGTACCCAGACACACCCTGGTGTCCTCGCTCCGCCATCTTCTAGCAGTCGTGGTTGAAA
MOUSE SEQUENCE - CODING
ATGCTCCAGCGGTGCGGCCGGCGCCTGCTGCTGGCGCTGGTGGGCGCGCTGTTGGCTTGTCTCCTGGTGCTCACGGCCGACCCGCCACCGACTC
CGATGCCCGCTGAGCGCGGACGGCGCGCGCTGCGTAGCCTGGCGGGCTCCTCTGGAGGAGCTCCGGCTTCAGGGTCCAGGGCGGCTGTGGATCC
CGGAGTCCTCACCCGCGAGGTGCATAGCCTCTCCGAGTACTTCAGTCTACTCACCCGCGCGCGCAGAGACGCGGATCCACCGCCCGGGGTCGCT
TCTCGCCAGGGCGACGGCCATCCGCGTCCCCCCGCCGAAGTTCTGTCCCCTCGCGACGTCTTCATCGCCGTCAAGACCACCAGAAAGTTTCACC
GCGCGCGGCTCGATCTGCTGTTCGAGACCTGGATCTCGCGCCACAAGGAGATGACGTTCATCTTCACTGATGGGGAGGACGAAGCTCTGGCCAA
GCTCACAGGCAATGTGGTGCTCACCAACTGCTCCTCGGCCCACAGCCGCCAAGCTCTGTCCTGCAAGATGGCTGTGGAGTATGACCGATTCATT
GAGTCTGGGAAGAAGTGGTTCTGCCACGTGGATGATGACAACTACGTCAACCTCCGGGCGCTGCTGCGGCTCCTGGCCAGCTATCCCCACACCC
AAGACGTGTACATCGGCAAGCCCAGCCTGGACAGGCCCATCCAGGCCACAGAACGGATCAGCGAGCACAAAGTGAGACCTGTCCACTTTTGGTT
TGCCACCGGAGGAGCTGGCTTCTGCATCAGCCGAGGGCTGGCCCTAAAGATGGGCCCATGGGCCAGTGGAGGACACTTCATGAGCACGGCAGAG
CGCATCCGGCTCCCCGATGACTGCACCATTGGCTACATTGTAGAGGCTCTGCTGGGTGTACCCCTCATCCGGAGCGGCCTCTTCCACTCCCACC
TAGAGAACCTGCAGCAGGTGCCCACCACCGAGCTTCATGAGCAGGTGACCCTGAGCTATGGCATGTTTGAGAACAAGCGGAACGCAGTGCACAT
CAAGGGACCATTCTCTGTGGAAGCTGACCCATCCAGGTTCCGCTCTGTCCATTGCCACCTGTACCCAGACACACCCTGGTGTCCTCGCTCCGCC
ATCTTCTAG
HUMAN SEQUENCE - GENOMIC
GGTGGAGGCTGGAGTGGGGGGCGCAGCTGAGACCAGAAAGAAACAGCTAGGACCCCACCTGAGGCTGGAGACACAGCCAGGACAGGGGACTCTC
CACCCAGAGAGACGCCATAGGCCAGGTTGTTGGGGACAGGCCTGGACTGGTCCCCAGGCCCCACCCTGCTGAGGCAGGCCAGCCAGCTGCCCAC
CCCTTGGCCTGGGCATGTCCAGCTTTTGCTCAGTTTCCCAGCCTGGCAGGGCTTTCCGAGGGGTTGCAGGGAGAAGCAGGCCTGGCCAAAAGAC
CGCCATGCCTGAAGGTTCACCTTCACCCCCTGGCACCACCAAAGACCCCGCCCAGGGGAAGTCTTGGAGCCTCAGCGCCTCAGAACTTTGGGAC
TTCCTGGCATATGGGAGGCACCCGGAGGTGTGAGCCTGAGGGGTTCACTTTCAAGGGTGGCTGGAGACACCTGCTGCTGGCCATGTGGGCCTGG
GAGGCCTTCTGGGGCCTCCATCTTCCCATCTGTGAAATGGGACTTGGACCTAGATGCTCTTCAAGGTCCCTTCTTCCAAGTCTTCTGGCAAGAG
CCAAGTCCAGGAAGCTCAGCTCCATTGCGTCGGCATCTGGACTGAGTACTCTGTGGGTGCCGGGCTGTGCTGAGGGCTGGACGGGACCCAGGAA
CAGACAACAGGTAGAGTGGAGGAGGGAGCAGCGAGGAGACTTCCCACAGGGGTGGAGACGCTGAGCACTCCAGGCAGACACAGAGGGGCGGCTG
GAGTGTGGGGATCCTGGGCCTGGCAGGGCCTGGGGGGTTCGGCAGGCAGAGTGGGCCAGGGGGTGGCCTGAGGACGCCAGGGGCAGAGCCAGCC
CTCCCAACTGGGCAGTGGGGAGCCAAGGGAGGGTGGCAGAGCCGAGCCCCATCTCTAAGATATGGGGACAGCCCTCAGAGCCAGCTCCCACCCA
GCTCCAGCTCAGGACAAGCAGTGGGCATGGAACTGAGATGCAGGAAGGCCGAGGTCTGGAGACGCACGTGGGCTCCCCGAGGTGACCTAGGGGT
GGGCGGCCCTCAGCCCTGCCCGGAGATCCCTGGTCCTACTGAGTCAGCCACCGGGGAGAAGATAAGGGACCGCCAGGCTCTTAAGGGGCCTGAC
GTTGCTGTTGGGTGAAGTGCCCGCCCAGAATGACCTCAGAGGCTGCCGGGGCCACCTGGCATATCACCCTGCCCGTCTCCGCAGCTTCCTCTGG
CAGTGGGTGGATGTATAATGTCCGCCCGCTGCCAGAGGGTTCACGGCACCACAGCCCTGCTGAGCCCTGCTGAGCCCCAGCTGGGTCCGACCTC
CTGGGTACCCCAGGGACGGGGAGGCCAGGCACGGGGGTTGCTGTGCGGGTTGGACAGTGGTGATGGTGGACAGAGCCACTCCCAAGAGTGTTAG
AGACCTGGGGTGGGGGCATCCAGGCAGACCACCCTCACCCCACCCTGTGGGACCCAGACCAGCTCACTTCCCTCTCTGGGCTCAGCTTCCTCAT
TTGCAAAATGAGGCCAAGAGTCACACCCGGCCCGGCCTGTGACCCCGGAGCCCCCTCCAGCCTCCTGCCTCACTCTGGCACCAGGCCTCCCTGC
AGCTTGCAGAGCAAGCGGGGTACCGTCTCCCCGAGGTGCAGCCTGGGGGGCTGTGGCAGGTGATGGGAGCAGCTGTATTTGAGGGCAGGGTCTG
TCGGGGAGCCCCCCAGCGTGGGGAGAGGAGGAGGGACCAGGGAGTGTAACCTGGCAGCCCGCAGCAGTTCCGGCTGCTGCTTCTGTCAATACGG
CCTTCCTGTTTTGGAGATTAAACCCCGGCTGAGCAAACAGCTTTCCAGGCGGCCCCTCCCTCCACCCCAGCCCCAGCCTTGTCTTCAGGTCCTA
AGGCTGGGACCACCTGGAGGCTGTGGGGGTGGTGGGGGATGGAGGGGACCAAGCTTTGCCATCCCTGCCCTCACCAAGGCCTGGACCACAGCCT
GAGCAGCCCAGCCCCTGCCCCTGCCGCAGCCTCCTCCCAGCCCCCCGCCGTGGGCTCCCCCGGAACCCCCCCGTGCGATCCTTCCAACCCAGGG
CAGCCTCACTGTCCTGGACGCCCGGCCACTGCATCCTCTGCCCTTGTGTGACCTCCTGCAAGAAGGCGGGAGCACGCCGTTCCCGGGCCTCATG
GCCAGACACAAGCCCACCTTGTGAGGACCCCAGGCGAGGCAGGAGAGCCACCGCAAGAGCCCCACCAAGGCACGGCCAGGGGAGGGCCCGCCAT
CTCTTGGATGGTTTCTGCGCCGTGGCCACTGACATTGAGGGTGGATCATTCTTGGTTGTGGGGCTGTTCTGTGCATGGGGGGCTGTGGGGCAGC
CTCCAATGCTTGGACTTTCCTCTGATGCCAGCCTCAGCCCAGCCTCTGCCGCCCTGAGAGAGACTCTGCCCCACCTCCCCACACCCGCCTCCAC
CCCCAGAAACCAAGACGAGCACTGGATAACCCCGTCTGGGCAAGTCCCTAGCAGATGGCGTGCACAGAAACGGGCTGCAGAGAGAGGACGGTGC
CTCCAATGCTTGGACTTTCCTCTGATGCCAGCCTCAGCCCAGCCTCTGCCGCCCTGAGAGAGACTCTGCCCCACCTCCCCACACCCGCCTCCAC
CATCCCCTCCTGCCAGGGTCCCACCCCTGAGCAACACGGCCCTGCCTTCTCTCTGCAAATCCTTCCCAGACTAAGCTGCAACACTGGCAGAGCC
TCCCAAGGAAGGGAGGAGGGAGGACAGAGGCCAAGGCGGCTCCTGGCCACCCTCGCAGCTTCCTCCCTCCCTGGCCACAAGGACCCAAAGCTCA
GAGGGATGGATGAACAGACAGGAAGGCTCAGGCTGGACACGTATTGTATGAGGTGGGCCTCAGGGTTGCTTTTCCTCCTAGAATGCCACTCAGA
GCCGTCCCTCTTGCTCGTGAACCTGGAGCCCCCTATGTCTCCCCCAGTACCCCTGCATTCTACACCTAGATCCTCAGGCTTCTCTTTATCTCCA
GCCTCACCTCCATGAATGCCCCACACACCTGTGCCTGACCACACAACCTCCACCTCCACCTGCTCCCACACCCCTGCCCAACTTCCACCTCCAC
CTGCTTCACACACCCCTGCCTGACTGCACAACCTCCACCTCCACCTGCCCAAGCCTTCAAGCCCCAGGGCAAATGTCACCATCTTTTCCTGGAA
GACTTTCCAGAAGTCCCCTGCCTCCCAGCCCCAGCCCACAGTGGGTTCTCCCTCGTCTGGGCCCTGGGCACCTTTGGGCTGGACCCATTTTTCT
AACCCCTGTTGAATCCTCACCTACTACCTTCCCCCAGTGCCAAGCAGATCTGGGCATGGAGCAAATGCTCAGGAAGGCTGTGGGATGAGCACAT
GAAATGGATGGAAGGATGGACGGACAGATGGACAGATGGATGGATGGATGGATGGATGGATGAGTGGAGCCCAACACCAGCTCTCAGGTCCTAC
GGAGGTGGCCTCTCTCAGCAGGTGTGATCGCCATTCCTGGGGACCTAACCTCCCCTTCTTCTGCTTCCAGAGCAGCCTGGAATATCCTTTGATG
CTGTATCGTCTTCCCTGATCCTTTCAAGGGGGAAAGATGGCCCATGGTAGCGATGGGGAGCCTGGGATCAGGGAGGTACACAAGCCCAGGATCA
CATAGCAAGCAGGGCTCCCCTGGGGCCAAGACTGACCCCTGCTCCCTCCACCCGGCCTGGAGCCCCTCCTCCCACTGCTCTGTATCCCATCCCA
CTGACATGGGCACGGGGTCATGACTAGGGTGGGGGGTCATGCCTGGGGCCTGACAGGCAACACCGTGGCCACAGCCCACCCCCTGCCTGCTGTG
GCTGCCACACAAGCCAGCCATTCTTGGCAAGACAAAGGCCGGCTGGGGCAGGGTGGCTCTGGTGCCTCCCTGGTGGGTGTCTGGACTCTGCAGG
ACGTCAGTAAGTGGATCCTGGTGGCTGCGCTCTGATGCTGGGGGCTGCAAATCAGATTAGCCCCATCCAGCCCCATGGCCCAGCTGGCCTCAGA
CAGAAGCGCCAGAAAGAGGCGCAGCTGCCAGCCCAGGCCTGCCGTCCTCCTGGTGTGGACAAGCCCTGGCCTCCAGGCTGAGGCCCCTTGCTGT
TCCTGCTGTGGGCTTAAGGGTGTGCCACCTCTTCTTTCTGGCCCCCTGGACCCGTCTGTAAGACCAACAGTTAGAGTGAGACCCCCTGAGGCCT
TCTGGAGGTGGTGTTTGGAAATGCCCCCAGGGCAGGGCAGGCACATAGTGCCTAGGCACTGGGAATTCACCTTCAGCACAGCCCTCCCCCAGGC
CCCAGGCCCTGTGCGGGCTCAGAGGCCAGCGGCAGCCCTGCCAGGTCTTCTCACCCTGAGACCTATGCTCCCCAGGGCTGGGCATTTGGGAGAC
AGAGTCAGAGAGGCGCCTTTGAGCCCTCTGTGTATCTGCACATCCACCCATCCATCCTTCCATCCAACCTCTACCAACCAATGTTCATCAAGCA
CCTGATACACACAGGCACTGCTCTAGGCCCTGGGGCACAGCCAAGAATCACATCACGGTCCTGCTGTCAAGCAGCTGACATCCAGCACCCTCCC
AGCCACCCGCATGCCTCTGTGTGTATGTGTCTGTCCATCTATCCATTAGACCATCTGTTGGTCCATCTGTCCATTCACCTGTCCATCCATCCAT
CCATCCATCCACCCATCCATCTGTCCATCCATCCATCCATCTATCCATCCATCCATTCATCCATCCATCTGTCCATCCATTCATCTGTCTATCC
ATCCATCCATTCATCCATGCATCCATCCATCCGTTAGTCTGTCTGTCCTTTCACCTATCCTTCCAGCCATCCATACATCCATCTATCCATCTGT
CCATTCACCTGTCCATCCATCCATCCATTCATCCATCCATGCATCCATCTGTCCATTCATCCATGCATCCATCCATCCATTCATCTGTCGGTCT
GTCTGTCCATCCATCCATCCATCCATCCATCCATCCATTCATCTGTCTATCCATCCATCCATTCATCCATGCATCCATCCATCCATTCGTCTGT
CTGTCCATTCACCTATCCTTCCATCCATCCATGTATCTATCCATTCATCCATCTATCCATCTGTCCATCCATCCATCCGTCCGTCCATCCATCT
GTCCATTCATCTGTCTATCCATCCATCCATCCATCTGTCCATCCGTCCATCCATCCATCCATCTGTCTGTCTGTCTGCCCATCCATCTGCCCCT
CCATCTAACTGTTCATCCATCCACCTTTCCATCCATCCATCTATTCATCCATCCATCCATCCGTCCCTCCATCTGTCCATCTGTCTATCCATTC
ATTCATCCATCCAACCATCCATCCATCTGCCAATCTATCTGTCCATCTGTCCATCCATCTGTATGTCCATTCCCCCCACCATCAGTTCAACCAG
TGTTTACTGAGCACCTACTTTGTGCCAGGCCCTGACAAGCAGCAAGAGGCAGCCTCTTCCCCCAGGGACACAGACGTAAAGGAGCAGCTACAAC
CTCATGTGATCAGCACCAGGATGGGGGTAGCAGAGGGGGATGGGGAGGACGTTCTAGGAGGGGTAGCCCTTGGTGGGCCCTGCAGGGTGCCTCA
GGCCAGGATGCCCCCCGAAGCCTTGCTCAGCCACTGAGGCCAAGTCGGCTGAACTCTAATGGGCCTGGCCTCTCTGTCCATGCCTGACCCCAGA
GTGCAGGCAAAAGGGCCAGGCCACTGTTGGAAAGGCCGTGGCTTCACGGCTGCAGAGCACCAACCCCAAGGCAGCCTGGTGGGCAGGGCGGGTG
AGGCACCAGCCATATCCCTATTACAGAGTCAAGTAACGTCCCAAGACCCACAGAAGGGCACTGGCCCAAGACCACACAGTGGGCGAGGGGTCTG
GGCATAGCAGGCAGGCAGGGTTGGGAATGGTGGGGGTGCTACTCACAGCTGGAGTCTGCTAGTCTCAGGGCAGTGCCCACTCCAGCTGCAGTAA
TGGGAATCAGCTGTGCCACCCTGCGGTATCCCTCCCCCGGCCTTGGGAGCCGGCTGTGCTGAGCCCAGCCAACCTTCCCATCACCCTCCTTCCT
GCCACCAAGGGCTGCTCACAGAAGCTCCTGGGTCGGGACACTCACTAGGCCACGCCTGCCTTTTGGAAATCCATGGGCCCCCTGGCATCTGGGG
GCTTCAAACACTGCCAGTTCTGATGCCTGGCCCCAGCCTGGGGGTTGGCATTCCTGCGTCTGTGTCAGGAGAGGACAGGGAGGTGCAGGCTTGC
CCGAGCCACAGCCACCACCATGCTGAGTCCTGTCCAGGAGTTTCCATCTCTGGGCACTGCTACTGGCCCAGAGGAAGCTGGTTCCCAAGACACT
GAGTGGGGCCGCTCTCCATCAGCCGATGAGTGGACAGGCGCCTCGGATGGCTTCCTGCCGAGTTAATGGGTCACCCATTATGTCTCCACAGATC
TTCCCAGGACCCTGCAGGAAGGCAGGCCAGAGCAGCCCCAAGGAACAGCCCGGGGGAGGGAAGGCAGATGGCCACCTCAAAGCCAGCCCCGAGC
CCTTCCCCTCTAGGCCTGAGTGTCAAGGGCAGACCCAGCCCATGGCTTTGGGGACCTGGTGGCACTGACTCCAGCAGGAGACCCCCCGGGCTGA
GCCCTGGCAGTTAGAGGAGGCCTGGACTCAGATGGGGACAGAACACTGTCTCAGCCTGATGGCGGCTAGAGGAGGAGGGTAGGGCCCCTGGTCA
TGTGGGTCCTGGCTCCGTGGGCCTGCTTTCCACCTGACCTCAGCAGGCAGGTCTCTCGGGCCATTCGCACACCCAGCTGCCTGGGAGCCCCAAA
CTGGGACTGGTGGAGGGGCCCTGCAGAGGGGAAGACTAGGATCCAAGAAGCCCATGAAGCCATGCGCCAGGGCCAGAGCTGGCCTGCCTGGTCT
GGATGGTATGTCATCCTTGTGTTACAAGTGAGGAAACTGAGGCTCAGGGAGTAACACCACCGGTTCAAGGTCTTAAAGCCGAGAAGTGGCCCCT
CTGGGTTCGGAAGGCAGGCTGGGCCCTGGGGCAGCCTCTATCCCCAGCCCCTCTTCCCCAGGACCCTACGGCCCCAAGAGGCTTCACTGGCAAC
TCGGTCAGCCCTCTCTAGCCCCCTCCCCAGCTCCCCTTTGTCCTGTCCAGGGCTGAGCACCATCCCACAGCCCACTTCCCTACCAGGGCTGTGT
GGCCCAGTGGGCCCCTAGAGGGAGACCTGGGGTCTGGATGGGGAGGGAAGTGTTTCAGGCTGAAGCTGACCCTGGGGAAGCGGGCGGGGGTGGC
TAGGCGGGCCACCCCCTCAACAGTTTTCAGACCTCAAGAAGTCACCACGAAATTCCATTCTCCTGACAGCAAAGAAATTGATTCACTCTTGCCA
AAGCCTGCGGCCCCCACAAGAATGCTGTAAAGCTTCCTCCCGCTGCTTCCAGACAGGCAGCAGCATCAGGTCCCCCACTCCTCCCATTCCTGGC
TACAGAGAGACGCGGGAACCCCCCCTACCCAGTTCTATGCATGTGCCCCTGCCAGGCTGGGCCATCCACCTCTGTATGGTCACTCCCTTGGTAC
AATATCCCCCTTGCTTTCTCTACAATGGGCACATTGCTCCCACGACCTGCTGGGTCAAAGGCACCGCAGTCAGGGGTAGCTGGGGAGCAGGAGT
TGCGGGGGTGGGGCGGCTCAGGGCAGGAAGCCCCATCCCCTGGCCTGGGAACTGTGGGGAGACGGACTCACAGCATCTTGTCAGGTGGGGTGCA
GGGTGGCATGGCACCCACCATCGGTGCCACTGATGTAAAGTCCAGGTACAGAGCACAACCCGTGCACATATTTGGGGGCTTGGCCATGCTTGGC
TGCAGGGGATTGAGGAGGGATTGGGCCTCAAGAAACGGGGCTGGGACTAAGTCACGAGGTGCGTGAATATCAGGCGGGACTTTAACTCAGCCAG
TGGAGAGCTATGGAGGGTTTTGCTCTTTGTGTGGGTGTGTGAGTGGCTATGATCCTAGTTGTCCAGCTGCTGCGTGGAGAATAAGTAAAGCAGG
TAGGATAGGAGTGGGTGGGATGGGGGTGGAGGCTGCCCAAGTGATTCAGGGAAGAGATGATGGGGATGGATGAGGCTCCGTCTCTGAAACAATT
CAGATATGGGAGAGCCTGAAGTCAGCGGTGTGACTGAAGCACGTGGGGTCATGGACACCCCTCAGTCCGGAGGCTGGGGCTGGGCTGGGACTCA
GCTGACTTTGTGGGGGCAGCCAACCCAGTCACTGGGTCCTTTTCTCCCCGGCTCACCCTCAAGCCTCTCCAGCTGCATGGAGGCTCCAGGGCTA
GGCTCTGGCTGATCGGAAGGGGTTTATGGCAGGAGGTGACGCCATGGGAGACTTGTCACTTGGGCACAGCCAGCACCATCTGCTGGGGCATCTG
CTTTCGGAGGTGGTGACTGCTAATTACCTGGGGCCAGGAAGAGGCCTGGGAACCCCCATCCCCCACACCACCCAGCGGGTACAGGGGCTAGGGG
TGGGGCCCCTGGGGCCCTAAGAGCAAATGGGCTACAGCGTCCTTAGCGGGGGGAGAGGGTCTGACAGTGGGGCCAGACATTTATCCAGTGAATT
TTTTCAGAGCACCTACTATGTCCCAAAACCTGTTCCAGGTCTGAATGACACCAGGTCGCTGCTGTCTTGCCGCTGACATTCAGGTAGAGGTAAG
GAGAGGGGCTCCAAGGGCACTTAAAGAATTTAAAATGCCATCTCAGGCGTGAACATAACTCCGAGGGGGGCAGTTTAGCCACGGTGGTGGCGGT
GGTGGTCGTGGTGGTCGCAGAGACCCTGCTTAGGAGGGCACGGGAAGACAGCGCAGCCACGGGCAGAGCCGCCTCCGTCCACGCTTCCGGAGGA
CGTGGCGGGCAGGGGGCTCACACCCTGCTCAGGCGACCCTTGGCCACTTCCGGGCCCCTCGCCCCTCACCCTCGGCTGCCGGCGCGCATTCCTG
CCGCCCCCGCTCGTGCGAGGGGCGGGCCCGGGCCCGTGGGAAGGGCCGGGGATGGGGGCCGTGTGCCGGGGAGGCGGGGTGGGGAGGGCGCACA
GCTGGGCCGCATCTGGCCCGAGCGCAAGGAGACCAAATGGGACAGAAGGCGGCCTTTCATCCGGCGAGGGAAGTGGCGGCCTTCGTTTCTCCTC
GCCATGGGGCGCCTGAGGATTTCGGGGAGCCAACCCTCCCGACGGAGGGCCAGGATCGAGGAAATCAGCCAGGGAGGAAGGAGTGGAATTTGGT
TCTGGTGCTTTCTCGTTAGAACCTCGTGTCCCCACCTGCGGCACAGGTGGCCAAGCTCTTGCTCTGTACAAATAACGGCTCCCCTGAACCGAGC
GCTCGGGAAGACCCAGGCGCCACCCCAGTTTGCAAGGCCTGCCGCGTCCCAAGGGGAGGAAGCCGTGCTCAGGAGGGGAAGCGCACAGCGAGGA
AGTAGGATGTCCCCCGCGGTGCGTCGGAGCCCAGAGCCCGGGTCTCAGCACCCTGCCCGCTGCGCCGAGGGCGCGCCCCTCCCGTGCCGGGACG
CCCGGTGCATCCCCGGACGGCGCTGGCCAGGCTGGGTTTCCTCTTCAGGGACACCGGCGCGGGCCGGGCCCGTCCCCAAGCCCCTCGGCGGCCC
GCAGGGGCTACCCGGGGCGGGGGGGGTGCGGGGCGTGCACGTGCGCGCCGGGGGCGGGGCCGCTGCGCCCTCTGGGCAGTTCCCAGCCCCCAGC
CGCGGCCCCACGTGGGTCTGCGGGCGTGGAGCGGCGACCGGCGCGAGGCCCCGCCCCCGAGCCCGGGACCCAATGAGCGCACGGGGGCGGGACC
GGGGCGGGGAGGTTTAAGAGCGCGGGACACTGGTGCTGCGCTGCTGGACTGCGCCGCCGGAGCGACGGGCTTCGGGTCGGTGCAAGGCAGGCGC
ACGGGGAAGGGCGCGCCGCGCGGCCGCCACCCCACCATGCTCAAGCGCTGCGGCCGGCGCCTGCTGCTGGCGCTGGCGGGCGCGCTGCTCGCCT
GCCTGCTGGTGCTCACCGCCGACCCGCCGCCGCCTCCACTGCCCGCCGAGCGCGGCCGGCGCGCGCTGCGCAGCCTGGCGGGCCCCGCGGGGGC
TGCCCCGGCGCCCGGGCTGGGGGCGGCGGCGGCGGCGCCCGGGGCGCTGGTCCGCGACGTGCACAGTCTGTCCGAGTACTTCAGCCTGCTCACC
CGCGCGCGCAGAGATGCGGGCCCGCCGCCCGGGGCTGCCCCCCGCCCCGCCGACGGCCACCCGCGCCCCCTGGCCGAGCCGCTCGCGCCCCGAG
ACGTCTTCATCGCTGTCAAGACCACCAAAAAGTTCCACCGCGCGCGCCTCGACCTGCTGCTGGAGACCTGGATCTCGCGCCACAAGGAGATGGT
GAGCCCCCCGCGGCCTGGACTGGCGGGCGAGCGGGGCGGGGACCCACCATCTGGTCCAGCTGGTGGCAGTGTCCCATGGGAGTCAGGCTGCATC
CCCATCCAGCCACTAGGGCCATCTGTGGGCGACGCCAGTGCACCCCGGTGCACCCAGTTTGCCTGCTGGGGCCACTCTCCCGTTACAGTTGTGT
TACTGTCCCCGGGGCCCCGCCTCCTGTCCAGCTCCAGAGTCCTGGATGGCTGCAGGACCCTACACCAGTCTCCAGTGCTTGGGGTTGGTGCCCT
GTGATACTTCTCACCCCGTAAAGATTGGAGCGCATTCATAGGGCATTATGTCCTCAAACTTCCACAAGCAAATTCTGTTCGGACCCACCCTCAG
CTCAGACCCCTTCCTGGGGAGAAAGGCTCTTCCTTTAGAGAAGTGAGGGGTTCTGACCTCTGGGCCCTTTCCTCATCACAGTGTTAAGGTACGG
GGAGGGAGGGAAGTCATTGCTGGTTGGTGGGTCCTGGGGAGTACATCTCAAAGAACTCCAGGTTTTGGGGTCTGTGTGGGGACCCTCCCCGCAC
AGTGCAGTAGTCTCTGAGGCCTGCAACCTTGGGCACAGCCCACTTCAGTTGGGCATCAGCTTCTTACACACACATAATTAAGACACACCCCACT
TTGAACATTTCCCTCGAGGCCACAGCAGGACGGTTGGGCTGGGGCGGGATGGGGACAGTGTGTGGGGCTTGCAGTCGGGGACTGGGGGGGTGTC
TCTGGGCTGGGAGATGCAGAGGCATTTGCTGTGAGGGTGGGGGCTCAGGGAAGACCCTGCACGCCTTCCTCCCTGCTCAAACCCCGGAGACCCA
GGGCCTTCTGACCTGGCGCTGTCCTGTTTTGGGGCACTGTGGGTGGGTGGGAGCAGGGCTTGGTGAGGTCATGAGGGCGACGGGGAGGGGCAGG
GTCCTCCCTGGTCCTGGCGTGGGGAAGGGGCCCTGGGAACCCCATGGCAGCCTCCTGGGAACACAGCGTCCCATTCCCAGGGGCTCAGCGGGCT
GGCGGGAGGGGGCGGCGGGGGCCGTGGGTTTTGTTTGGCGGCCGGGCCGTTAGGATTCCCAGCGCCGGGCGGCTCTCACGCCGGCCCCTTGGGC
AAGGTAACAAAGCCCCTTTCTCCAGCGAGAAAGGCGTCGGGCCCCTTGTGTGCAGCTACGCGCTCCGTGGGAACCGCTGCTGGCGTGCTGGCCA
CTCAGGTTGCCTGGCGATGAGGGGACCAGCGGGCCCCCCCAGCATCTCTTACTGACAGTCCTTTTGGGGCACCATCCCTGAGCCTGGCCCCCCC
ACCCCCAGCACTCAAGCCTTACTGCTATGTGATCTTGGGCATCCTTCAGCCCCACTCTGGGCTACCACCAGCTCTGAGCACCTCTCATCTTGGA
ATTTGGGGCCTTTCTCCAACAGAGGGGATGTGGTCAGAGGGCTGGGCTGTGTGATCCTGAGCAAGACCTGGGGCCTCTCAGACCCTCACTCTGA
TCTCTGAAATGGGGGAACATGGTCCTTCCAGGAGCACGTTGTAAGAGACTGGCAAAGCCTGCAGGCATGGGGTGAACCCGGCGGGCACTCAGGG
TCGAGGTGGGGGCCATCACTCCCTTGAGCCCTGAGGTCCCTACTGGAGGGTCACCCCACCAGGGGACAGGTTGAATCTGCCCTCAGCATCAGTT
CACCAGCACACAGCAAGCAAAGACCCCCTTGTGGTGGGTGATGGGCAGGGGTCCTGGAAACCCACAGAAGAGGGCCCCCCTCTCCCAGCCCTGC
ACGGAATGACTCAGACATGGTCTGATGGGGGAGATAGAAAGATGTCAGGGCTGTGCGGGGGGAAAACCCCGAGAGGCTGTGTGAGGCAACAGTG
GGCATCTGACCCACATGCTGGGGGAAGGGAGGGAGGAGGGAAGACTTCCTGGAGGAGGCGCCATTTGAGCTAGACTAAGAGTAAAAAAGTGCCC
AGAAAGGGCACTCCATCCGGGCATGGTGATGACCACAGCATGGGGGTTGTGCAGGAAACTTCTAGCAACTGGAGTGGTGGCTGGGGGGAGGGTC
GGTTGGTCAGCAGGGGCCGCGCTGAGATGGGCCTGGAGTCCAGGATGAAGAGCCTGGCCTTTGGCCCAGAGGTTCCCCGCCCCTCCCAGGAGCA
GCCAGGCTGCCGCGCTGCCACCACAGCGGCCACAATTGCTGGGGAGAACTTGGCATCCTGTGTTTCTTTGAATCGCGGCTCCAGGAACAATGCT
GCTTCCTGTGGGTGTCCCCCGGCCCCCGCCCCCGTCCACCCTCATCCACCCGGCTCTTCCTCCTGCAGCCCCGTCTTCGTCTGGAAAGTTAGGT
GGCACTGGGGGGTCTGGGTCCCTAGAAGGCAGGGACTGGGCTGGTCTAGCACCGCCTCGGCCCCAGGGTATGACCTCAGGCTGGTGCCTGCACC
TCTCTGGGCCTCAGTTTCCCCACTTGGGCCTCAGCTGGTAATGGCACCCACCTCCGAGGGCTGTCAGGGAGAGGAAGGGAGAGAATCTGTCTTT
GCACTTTGTTCTCTGCCCAGGGGAGCTCTGTTATGCCCATTTTACTGAAGACGAGACTGAGGCTCTGAGCGGCAGGTGAGGGCTATAGCCCAGC
AGCTCCTTTTTCCTCCCCTCCTGGTGGCTCAGGTGTCGGGAGACAGTTCGCCCGGCACCCCCGCACCCCATAGCCCCGCCTTCTCAGATGCTGT
TCTAGAGGCCTCCCTGCTCCCTGAGCTGAACAAAGGTGACTTTAATCTCCGTAGCCTTGGGCTGGGTTTTTAGGGCACCACCAAATCTCGGGAG
AACAGGCCTCCCTTGATCTCATTAAGAAAGCATTTTCCTTTTAACCCAGCGGCGCAGTGGAGCGTCTGCAGGGGCTGGCCCTCCGCCTCCCACG
GCATTCCTGGGCTGAGATTCTGGGGAGGCCAAGGGGCTGGGTGCGAGTCTGTGCGTGTGGCTGCCCTGGGGTGTCTATGGGTGCTGCAGGAGGG
GTGCATGCATGGAGAGGCGGAGACCCAGCCTGCCACCCCCTTAGCTCTGGGCCTCACCCTAGTGAGCATGTGCCCAGGGCTGGGTCAGGCTGGA
TAAGGATCTGAGACTACCAAGGTGTTCCAGGGCCTCTGCGGGACCTGTGTGTACAGGGAAGGAAACTGAGGCTGGGTGGGCATCAGGGGGTTGT
CCCTTGGTCAGAGCATTGCCCGTACACCCTGGGTTTTGCACTCAGCCATGCCAGGGTGCGCCGGGCGGCACGCGCTGGGCGGGCCGGGCAGAGG
AAGGAACATTCCTCCCCGTGTGCCGGCAGGCCCAGGCGGGCGCCGGCGACTCCTGGCCCCGCCCTGGCGGCCCCCTCCTCTTCCGCAGAGCGCT
CCTGGCAGGGGGCGGCCGCGTGCTGGCACAGGTGGGCTGTGGCCTTGGGCAAGCCTGGGTTTCCTTGCCTGAGCAGTGGGAGGGGTGCTGGGCT
GCATGGGTACTGGGGGCCCCTGACCTGGGGGCTGGGGACACCTGAGGTTTCCTGCCACAACCCTGCTGACCGTTGGCAACTTTGCAGAACTTCA
GGGCTGCTTTGGCTCAGAGTGGGTAGCTTGTCCTGCAGGTCACCCAGGACAGGGGTGCTGGCCCAAAGGGCAGCTGTGGGGTGGCTGCAGTGCT
GGTGGCTGCACCCTACGCGCGGGACCTCATGCCCAGCCTGGCCCGGTTAACCAGGCCAGCTGTATGGTAATGGGCGGCGCGGCCACTCTGCTTA
ACTCGGCCGAGTTAAGGCCGGCCCGGCGCCCCTCTCCCGCCAGCCCAGGACAAAGCTGGCGGACAATGCCTGGTGATTCATCCTCCCTCCAGGC
CCTTTGTCCGCCAGGGCTTGGCAGCCGCCCAGCGAAGGCCTTTGTCCCCAACCCCGAGTGATTGAGACTTGGCTTCCCACAGCCGACCTCCTCC
GCCTGTCCCTCCCGCCACCCCGCCTGTCCCCCGCCTTTCCAGCAGCCTCCAAGTGGCAGGCTGGGTGCTGTGTCAATAAGGGTGTGTGCCCTGC
CAGGTGCACGGGGCGGGTCGGGAACCGGTTACCTGGCGGGCGGGCGAGGCGCACCACCTGGCTCGTACCTCACCGCGCCTGGACGACCTTGGGT
GCAGCTGTCGAGGTTGGGGAGTGAGGCCCCCATCCCCCACCCCCAGGGCGAGTGAATGCGTGAGCTCTGGCCCAGATGGCACTGAGATGGGGCA
CTTGAGCCCTTCTCAGCACGAGTGGGGAAACCAAGGCCCGGAGAAGGGTGGGTGTCAGTGTGACGCAGCTGCAGCTGCAGCAACTCCAGGGCGC
CCCGTCCGGCCCCCACAGATGGCCCTGGGGTGGGGATGAAGGGCTGCCTGCTGAAGGCCGATTTTCTCCTTCCAGACGTTCATCTTCACTGACG
GGGAAGATGAGGCCCTGGCCAGGCACACGGGTGAGCCCTGGACTTGGGGCGGGAGGGGGCCCAGGCCTCCATCCAGAGCCGAACGCTCCCCCTC
CAGTCTCCCTGCCCCTCTGGGCCGAGAGGTCACCAAGGGCAGGACAGGGAGGGCAGTTTACTCATGGGGTTTGCTCCATGCCCCGCCCCACCAC
TCGGGCCCCCCAATTCTCATGCAAATGAAGCCCATTCAGCCCCCCGGGTCCCTTTGAGCCCAGGCAGCGGCAACAGGTGGCGGGTGCTGGGAAA
AAGCTGCCTGAATGGGCGGGGGCGGGGCCGCCCTGGAAGGGGCTGGGCTGCGGGAGGGTCCACAGGCCCAAGCCCCGAGCTAGGGTGGGGGAGG
CTACGGCCGCCGGGCCCAGCTTGAACTAGGGGGCTTTTCTCGAGCCCCTGGGCCCTGTGGCGTCCCCCAGGCCAGGCCCCTCTCTGGGAGCCGG
CTCAGACCTACTCACAGCCGCTCCCCTGTCCACAGGCAACGTGGTCATCACAAACTGCTCGGCCGCCCACAGCCGCCAGGCGCTGTCCTGCAAG
ATGGCCGTGGAGTATGACCGCTTCATCGAGTCCGGCAGGAAGTGAGTGTGGCCCCGGGGGACCCCCATCTCCCTGCCCGAGCCTGGCAGCGCCC
GCCCCGGCATGCCCTCCCCCGATGGCCCTGCCTTGTCCTCAGGTGGTTCTGCCACGTGGACGATGACAACTACGTCAACCTGCGGGCCCTGCTG
CGGCTGCTGGCCAGCTACCCGCACACGCGGGACGTCTACGTCGGCAAGCCCAGCCTGGACAGGCCCATCCAGGCCATGGAGCGGGTCAGCGAGA
ACAAGGTGGTGAGTGCCTGCCCCTCCCAGTCCCCCACGCCCACGCGGAGCGCACACCCGGAGTGGGGGTGGGACAGTGAGGGGCAGCAGCGCCT
GGGTCTCAGGACACCTTCTCCCTTCTCCCAGCGTCCTGTCCACTTCTGGTTTGCCACGGGCGGCGCTGGCTTCTGCATCAGCCGTGGGCTGGCT
CTGAAGATGAGCCCGTGGGCCAGGTGAGTGCCCTGCACAGGTTAGGCCAGCCCGGTCCCAGGCTCCTCGCCACTGTGGGGCCTGGCTTAGTTCA
TCTTCCCAGCCATGGGGTGTCCCCAGCCTCCTGTGTGGCACTGCCCACTTACTTCCTATATTCCACTTCCCTCTGGGTTTCAGAGGGCAGCTGT
GTTTACGGCGGCTGCCCCCAAGCCTGACCTGCTCAGAGCAGCCAGGGGGGCGATGAGCACCCCAGGCACCATCCGGCAGGACTCTTCCCTGCAC
CCAGATTCCCTCCACAGAGAGCCACGGAGCACAGGAGCTGTGCAGGGAGTGTGCCCTGGCTGTGGCCAGGGGAGGCAGAGGGAGCTGCAGCCCA
GAGCTCTCCTCAGGGCTCCTCTCCCTGAGGAGTGCAGCGCCTTTGCCTGGTGGGGCCTCCCCAGCTCCCAGCAGATGGCTGGGGCCTCTGCTCA
CTGGTCTGGGCCCTTCCCTCCCGCAGCGGGGGTCACTTCATCGGTACGGCTGAGCGGATCCGGCTGCCTGATGACTGCACCATCGGCTACATCG
TGGAGGCCCTGCTGGGTGTGCCCCTCATCCGCAGCGGCCTCTTCCACTCCCACCTGGAGAACCTGCAGCAGGTGCCCACCTCAGAGCTCCACGA
GCAGGTGCACCATCCTCCGGGCCCCGCCAGGACTCCGAGAGCACAGGAAGGGACGTGTGGCTGCCGAGAGGGGCGCAGTGGGGTGGGGCACTGT
TCTAAACAGGGAGGCCAGGCAGCACTCCACTGTCAGCCAGGGGGGGTCACTCCTGCCATGAGCTCAAAGCTGTTTATGGCGGGTTGTTTTCCTG
CCGTCTCTTCTCTTCACTGTGATGCGCCTACTGTGCGTATTTTGTCCACGCTGGCAATGCAGCCAGCCCCAGCTGGTCCCCAGTTTGGGACCTT
ATTCCTGGGGTGTGCAGGGCAGGTGTCCTTCCAGGTCCAAGGGAGGCCAGGGCAGGGCCGTTGCCTCACTCAGGGCTGTGTGGCCAGCCTGGGG
CGGGGCCCAGGGATGTCGGGCCCCTCCCGGCATCACTCCGCCCGCTCCCCACAGGTGACGCTGAGCTACGGTATGTTTGAAAACAAGCGGAACG
CCGTCCACGTGAAGGGGCCCTTCTCGGTGGAGGCCGACCCATCCAGGTAAGGAAACCCCGGCCCAGATGGGCTTGCGTAGGGTGGCCTAGGGGC
GTCAGGGGGCCTCGTGGAGCTGCAGCAGGGTCTCTCTAAGCGGCATGACTCTACATAGAGGTGTCCCCCGGAGTCCTGCTTGCTCGGGGTGGGG
CCGCCAGTGTTGTGGGACTGCAAATGGGAGCTCAGCACCTGCCTGCCACCCACGCAGACCAGCCCCTGCTCTGTTCCCACAGGTTCCGCTCCAT
CTGCTCCGAGGGCCAGTGGGCTGCAGGGCCTGCTTGGAGGAAGGATTTGTGTGTCGGAGGCCACTCCGAGGGCAATTCTGTTAGGATTTTTGGA
TCTTTCTACAGCTACGGGGCTCCGGGCTACTTTGCAGGGATGCGATGCGTAGGTGCCTTTCTCTTCCTGCTGACCACAAGCTCTGTGCTGGGGG
TACCTGTGCCCTGAAGTCCTGGCCCCTGTGTCAAGCCCCAAGTACGACTCACTGAGCCATGCTCATTGCAGGGGGAGGCTGGGTCTGGGGGTGC
GTGACCCAATCCCCTTCCTCCTGGCCTGGACGCTGTGGCTTAAGAGTAACAGCAGCCACCGCCCAGTTCCAGTGGCCCCACGAAGAAAACAGTG
GCTGGCTGTCCAGCTGGGCAAACAGTGGCACCCCTCCCAGCTCTTCTGAGTGGGGAGTCTTCCAGGCCTCCTCAGAGGTCTTCCCTTTGCCTCC
CCAGGACAGGGTGAGTCAGAGCTCAGCATTTAATCTCCTCTCCAAAGTAGAGAGCAAGTCGCCCACAGTGGGCGTGTCTGTAAATATTGTGACA
GTATTTTTTTACTGTGCTGTTTTTTTTCAAAGGGGATGGGTAAAAAGTAGGGTGTTCTTGTTTTTTGCTTGGGAGGGTGGGGGTGGGGGAGGGT
CTTATTTTATCTTATCTTTTCTGTGGATCAGAAAAAACAGAAGCCAAACTCGGGGTCATCTTTGTTTTTAAAGCTGAAGTGGGACTGTCTGGCA
CTCTGTGTATTTATGCGTTCCAGCATCTGGAACCTCCCATCCCTGCCCTCCTCCTGTGTAGCTGCCACCTCCCCGCTGGGCCCAGCATGGCTCA
CCTGTCCCGTGGGCTGTGTTTCTTGTTGTTTTTCTCTTTGCAAAGACATAGCTAGGAAAGCGAATGATAAGGGAAAAGTTCTCAGGGAATTGAA
GTGTTGTTGCTATGGTGACGTCCTTTTGCTGTGAATAAAGGTGCTCTTTGCAGCAATCCTGGGGGTCCTGGTCTTGGCTGGAGGTGGGGCCTAA
GCCCCAGGGCAGCCAGGGTCAGGAGAGGCACACAGGTGAAGGGGAGGCTTGGCCTTGGGTGCACCGCATGGAGCAGGTAGATAGGAAACGCTTA
GAGACAACTATTTTGGAACGAAACCTGGCGTCACTATAGCTGCCTGCTGGTGACTACAGGTGCGGCCCACCCAGGGTCAGGGTGGTCGCTGGAG
CGTGAGCCCTGTGCTTCCAGGAGGAACAGGTCTAGGATGCTGGGGGCTCTAGGTCCCAGCCAAAAGCACAACCCCAAGGCTCTGGCCTGGTCTG
GCCACTGGGGAGCCCAGCCCCTGCAGGAGGGACAGTGACTCCTGTGACCGGGCCTGGGGCCTCCTTCTCCAACTTCACTCCTGCCACGAGCTCA
CAGAGTCCACATCATCGCCTCACTTTGCAGGTGGGGAAACTGAGGCACGGAGCAGCTGATGCCCAAGGTCCAGCAGCCATGCTGGTGGGCTGGG
GAGGCGCTCAGACTCCAAGCCAGGCAGGCCGTGGGTCCGGCCGCCCCACAGCTGGTAATGCTGCACCTCCCTCTCCTGTGATGTGGCCCCAGGC
CCCGACCCCGCTCAGGGGTGGTGGGAAGAGTCCTGGAGAGCAGGAGCAGTGCCGGAGCCCCCACTGCAGCTGGTCCCAGCCCCTGCAGAGATCC
AGACGCTTGTTTTTTAAATACAGCAGCTCTGCGGTGCTTTTCCTTCCTCAAATCCAGCCTTTGAGGGTGACTGAGTAGGGGACGGGTGCTGCAG
CCTCTGCGGGGATTCCCTCCCCTCACCCTCCTGGCAGCCCCGGGGCGGCTCACTTCCTCTTCACCATGAGCCCCCACCCCTTCCCTGGGGCTTA
CACCCTTGGCCTGGCACTGCCCTGGCCGCGCCCCTTGCAAGTTCCTGCTCTGGCCTGACCCTGCTTCCCCCGACCCCAGGGCTCCAGGTCCCCC
TCCTGGTGCCTCTGCACCCCCCACTCTGCTCCAAGGAGACGACACACTGTGAAAAGTGCCTCTCTATGGGGGGGTGCCTTGCCCTCCCCCGTCG
CTGCCCCCTCCAGAAGAGTGCTGACGTGCTGAATGGGCCTGCAGAGACCCCCTTACTCTAAGCCTGGCTGGGGGCTACCCTGGCCTTGGGGGCG
GCTGGCAGGGCTGTGACCCCACCTTGGCCTTGGACGGTCCTGAGTCAGCAGAGCAGCTGCCTGGGGGGTGTGGACAGTGATGGGGATGGCGGGA
GGATGGAGGGCCCTCACCGTCACGTGGCCTGACCCCCGTCTGGGTGGCTGTGGTCCTCCCTGAGCACCCACCATGTGCCCACCCACTTCTCATA
GGACGGCATAGGCCCCTCTTCTGAACGAGGAAGCTGAGACTCTGGGGTGGGGCGGGGGGGGTCGTGACGGAACAGGCTGCGGGCAGAGGCTGCT
GGCCTCCTTCCTGCCCGGCTGACTCAGAGGCTTGGGGCCTTCTGCCCTGGCCCCGACGCTGTCCCCGCCTGCAGCTGGCATCGCCTCCCCACAG
AGCTGGTGGGGCCCCGGGGGCAGAGGTGTGGCTGGACCCAGGGGTCGGACAGACCCATGGATACAGCTGTGGGGGCCCTGCCAGGGCTTGGGGA
GTCCCCATTAATGTTCATATGTCCCTGTGTGTGGAGCGGGGTGGGAGTGGGGGCTAAGTGGGCATCGGGGAGAGCAGAGGAGCAAGAAGTGGGG
TGGGGAGGAAGCCCGGCTCCTCCCGAGGCCTCGACCTCTCCATCTGACAAATGGGAACAAAGGTAAATGCAGTCCCCGGGTGCTGGAGGTCAGG
GGGTGTTTTGGAAGGTGCACAGGACAGCGCATCTGGCCCCTTCCTCTTCCAAGCTGAGGGAGGGACGTTTCTCTGCCCTCAAAAGGGGAAGCAG
GCCCAGCAGTGTCTTAGGGGAATGAGGCAGCCTGACCTCTTCCATCCACCGCCCCCCCACCCCCCAGAAGGTGACTCACCCGGCAGGGGAGGTG
AAGTCATTCCTCCCCCAGGTACATTCTGGGGACAGCTGTGTCCCACCCACCAGTGGTGCCATCACATCAGATTAGAGGTCTGGTCAGAAAGGGG
CCTGGCTGGTCCCTGGGGCTCCATGTCCCCCCAGTCTAGCCCAGCCAGGCCCCCTTTCCCAAACTTCTGATCATTAATGGGGCTTTCAAAACAA
GTAAAACTGCAACCACAGGGGCTGCAGGGCGCTCAGCGAGGGTGGCAGACCTCAGCCGCCACCCCTGCTTCCGTTCTGCCAGGCGAGTGCCTGG
GGAGCCCCAAGGCTGCTTGGGGTCTGGGAGGGGCTGCTAGGGTGGGGCGCCTCCCCACTCAGTTGAAAGGAACTTCCCTTCCACTGGGGCCATC
AGGGCCAACTTCCTGGAAGAGGTGCCTCTGAGCTGAAGCAGGAAGGCTTTGTCAAGATGGAGTAGGGAGAGAAGGGGCCTTCCAGGTCTCAGCA
CAGCCTGGGCAAAGGTGCAGAGGTGGGATCCACTGGGAGGACTGGAAGCCTGGGAAGGAGGGCAGGCAATGCAGTTGGGTCTCTTGATCCCCCC
TCCCCAATCACAGACAACTAGGGCTCTCCACAGGCTCAGAGGGGCCAGGGCCAGGCTGCTATGTGACTTCAGCCATTCTTGCCGGGTGGGCCTG
GGGTCAGAGGGAAAGCAGGGACCAGGGCCAGATCCATCTTCGAGTCTGTCAGAACAATGGGCAAGTCTTGTTCCCTGCTGGGTCCATGTGCCCA
CACAGCCTCAGCCTTCTTAGATATTCTGCAGGGTGACCCCCTGCTTCTGAGCCCAGCCCCACCTTCTGAGCCCAGAATTGGACACACAGGAAGG
CCCTTGGGGCCAGGCGTGGTGGCTCACAGCCGGGCTGTGCCCTCTGCCTGGAGCCGCAGCCTGGATGCCTCCCACCCCACATTTAGGCTTCAGG
CCCCACAGCTGCCTCTTCCCAGAACCCTCCCGGCACGCACTTCGGCGACTCTTACAAGTACCTGTTCAGCGTCAGCCTTCGCCCAGGAGTGCAG
GCTGGGGACGAGGGCAGTGGCCACCTCTGCCGGTCCTGTCAGCACCTAGTGCAATGTCCAGCCCACCGTGGGCACTCAACGCAGGCGGCTGTGC
TGCCGCAGGTCACAGCACCAATCAGACACCCCCATCCCCACACACACCCTGCACCGGCCCACGCGGCCATCACACTCGGAGCACACGCGCTCCG
ATGTGGCTGCACTGTTGGCACACGCACAGGCGCGCACGTGGGCGCATACACGCCGGGGCGCCCTGACCTCCCCGCACGCCCTCCCGCAGCCGCC
CCGGCCTCTGTCCCCACGCGTGTCACACACGGCTGGGTGTGCTCGCTCACGCCGTCCCGCGACGTTGCTCCAGCCGGCGCCCGGAGCTCGATCG
GGAGAGCTGCTGTTGGCCGCCTCTGCCTGCCAGCGTTTGCCAAGCGGTTTCCATGGTGACACATCTTGGCTCCGACAGGAACCAAGGAGTGTGG
GGAAGAGGGTCCCCACGGAGGGGGCGAGGCGAGGCAGGAGGTCTCTGCGTCTTCAGGCTCCCGGCAGGCATGGGGGGCTCCACGAGTGCAGACA
CCCCCCAATCCCAAGCCAGGCTTGCACCCCGAGAAGGAGGCTCTGGGCTGGTCCCTCTGGGCTGCCTGGCACAGGGGCTGACTTAGGTCAGTCA
GTCATTCAACAAACTTTCTGGAAGAGCCACTCTGTGCTCACAGCCGGGTCTACGATGCTGCCCAAGACGTGGTCTCGCAAGAGGCTCGGAGTCG
AATCCTGAGGCTAAACTGACGTCCCGCCACCCTACTGAAGGCCCCATCTAGCAGGTTGGTCCGTGGCTGCGTAAGCACCAAGGCCCACCGAGCA
CTTTCTCAAAGGGGGGCTCCCCTCTGTGGTAGATCAGTTACAAACGTGTCCCCGATGCCCCACTCCTCCCCTGAATCACCACGCCCTTTGCTGT
GTGACTTTGCCCCAACTCCTGTCTAGAGGTGGCGTCTACTTCCCCATCCTTCAATCTGAGCCTGTCTTATGACTTGCTGTGGCCAATAAAAGGT
GACAGCAATGACATTCCCAGTTCTGAGCTTAAGCCTCCAGGGACATGCGTGCCTCTGCCTGCTCTCCCCGACTCCTGCTATAGCACGAGAACGG
GCTGGGCTGCTTTGCTGGAAGGCAAGAGGGCCATGGAGCCTGGGTCACCCTAGCTGAGGCCATCCTGGACTAGCTAGCTCCCAGCTGCCCACAG
ATGTGTGAGTGAGCCCAGCTAGGACTTGCCAGGCCTAGCAATGAGCAGCAGTACCACCAGGCCCTTTAATACTAAGGACATGTGAAGTCATGTG
TTTGGAGCTATTTCTTACTCAGCACTAGCTACCTGATACCCCTCTCTCTAGGGCCCTGGCCAGCAGCTGCCCAACCTCAGTTAGCACAGGCCAG
AGGCTCTAAAAGTTCCCAGGGGTGCTAAGAACCAGGCCATGGTGACAGCTGGATTCCGCTGGCTAACAGTGCCAGAGGGTGCCGGGGACAGAGC
TTGTGGGAGGGGGTTCCCATGTCAAGTCCATGGCAGGAGCTACTGAAGCAGAAGCACGAGTCTCCACTGCCTTTCTTTGCCTCGGTTTTTCCCC
CTGTGAAATGGGCCCATCACCCTGTCCTGTCTCCTGGGGACCCCTGGTAAGGTAGCGTGTGTGGCAGCTGGAAACAGACCTACTACGTGCCAGC
CCCGGGTTGCAACCTAGGGAGAGAGGCCCATTGCTGCCTTCTAGGTTCCCACAACTGAGAAGGCAGCGTCACTAATCCCAGCTTACAGACTGGA
AACAGTGGGGAGAGGATGAGTGGCTTGCCCGAGGCCCAGGACACACAGGAGCAGGTGGGCATGGCCACAAGCCCCGGCTGGGTCTGAGCTGGTG
GTTATCTTCATGTTTTTCTCCCTGTGGTCACCTTTTTGGAAATTTTTTTTTTTTTTTTTTTTTTTTTTTTACATTTGACTATTTTCTTGTTTAT
TGGTATTTCTTTGGGAGATTTTTTTTTTTTTTTTTTTTTTGAGATGGAGTTTCGTTCTTTTTTTTTTTATTTTTTTTTTTTTTATTGATCATTC
TTGGGTGTTTCTCGCAGAGGGGGATTTGGCAGGGTCACAGGACAATAGTGGAGGGAAGGTCAGCAGATAAACAAGTGAACAAAGGTCTCTGGTT
TTCCTAGGCAGAGGTCCCTGCGGCCTTCCGCAGTGTTTGTGTCCCTGGGTACTTGAGATTAGGGAGTGGTGATGACTCTTAACGAGCATGCTGC
CTTCAAGCATCTGTTTAACAAAGCACATCTTGCACCGCCCTTAATCCATTTAACCCTGAGTGGACACAGCACATGTTTCAGAGAGCACGGGGTT
GGGGATAAGGTGACAGATCAACAGGATCCCAAGGCAGAAGAATTTTTCTTAGTACAGAACAAAATGAAAAGTCTCCCATGTCTACTTCTATCCA
CACAGACCCGGCAACCATCCGATTTCTCAATTTTTTCCCCACCCTTCCCGCCTTTCTATTCCACAAAACCGCCATTGTCATCATGGCCCATCCC
CAATGAGCCGCTGGGCACACCTCCCAGACGGGGTCGTGGCCGGGCAGAGGGGCTCCTCACTTCCCAGTAGGGGCGGCCGGGCAGAAGCACCCCT
CACCTCCCGGACGGGGCGGCTGGCCGGGCAGAGGGGCTCCTCACTTCCCAGTAGGGGCGGCCGGGCAGAGGCGCCCCTCACCTCCCGGATGGGG
CGGCTGGCCAGGCGGGGGGCTGATCCCCCCACCTCCCTCCCGGACGGGGCGGCTGGCTGGGCGGGGGGCTGACCCCCCCACCTCCCTCCCGGAC
TGGGCGGCTGGCCGGGCGGGGGGCTGACCCCCCCACCTCCCTCCCGGACGGGGCGGCTGGCCGGGCAGAGGGGCTCCTCACTTCCCAGTAGGGG
CGGCCGGGCAGAGGAGCCCCTCACCTCCCGGACGGGGCGGCTGGCCCGGCGGGGGGCTGACCCCCCCCACCTCCCTCCCGGACGGGGCGGCTGC
CGGGCGGAGACGCTCCTCACTTCCCAGACGGGGCAGCTGCCGGGCGGAGGGGCTCCTCACTTCTCAGACGGGGCGGTTGCCAGGCAGAGGGTTT
CCTCACTTCTCAGACGGGGCGGCCGGGCAGAGACGCTCCTCACCTCCCAGACAGGGTTGCGGCCCAGCAGAGGCGCTCCTCACATCCCAGACAG
GGCGGCGGGGCAGAGGCGCTCCCCACATCTCAGACGATGGGCGGCGGGGAAGAGGCGCTCCTCGCTTCCTAGATGGGATGGCGGCCGGGAAGAG
GCGCTCCTCACTTCCTAGATGGGATGGCGGCCGGGCAGAGACGCTCCTCACTTTCCAGACTGGGCAGCCAGGCAGAGAGGCTCCTCATATCCCA
GACGATGGGGGGCCAGGCAGAGACGCTCCTCACTTCCCACACGGGGTGGCGGCTGGGCAGAGGCTGCAATCTTGGCACTTTGGGGGGCCAAGGC
GGGCAGCTGGGAGGTGGAGGTTGTAGCGAGCCGAGATCACGCCGCTGCACTCCAGCCTGGGCACCATTGAGCACTGAGTGAACGAGACTCCGTC
TGCAATCCCAGCACCTCGGGAGGCCGAGGCTGGCGGATCACTCGCGGTTAGGAGCTGGAGACCAGCCCGGCCAACACAGCAAAACCCCGTCTCC
ACCAAAAAAAAAAACGAAAACCAGTCAGGCGTGGCGGCGCGCGCCTGCAATCACAGGCACTCGGCAGGCTGAGGCAGGAGAATCAGGCAGGGAG
GTTGCAGTGAGCCGAGATGGCAGCAGTACCGTCCAGCTTTGGCTTGGCATCAGAGGGAGACCGTGGAAGGAGACCGTGGAGAGAGAGGGAGACG
GAGAGGGAGAGGGAGAGGGGGAGGGGGAGGGGGAGGGGGAGGGAGAGGGAGAGGGAGAGGGAGAGGGAGAGGCTGTGTGTCAGAGGTTTTCCTC
GGAGTTTCGTTCTTGTTGCCCAGGCTGGAGTGCAATGGCACAGTCTTGGCTAATTGCAACCTCTCCCCTTTTTGGAAATATTGTACTTCCTGAG
CTAACTGCACTTACGAGGTAATGATTGGTGTGAGTTTCTAGTCTCTGTTTGTTTGTTTTTTGAGACAGAGTCTCATTCTGTCACCCAGGCTGGA
GTGCAGTGGCACAATCTTGGCTCACTGCAACCTCTGCCTCCCGGGTTCAAGCGATTCTCCTACCTCAGCCTCCTGAGTAGCTGGGATGACAGGC
ACCTGCCACCATGCCCAGCTAATTTTTGTATTTTTAGTAGACAAGGTCTCACCATGTTGGCCAGGCTGGTCCTGAACTCCTGACCTCAAGTGAT
CCACCCGCCTCGGCCTCTCAAAATGCTGGGATTACAGGCGTGAGCCACTGCGCCCCGGCTCGCTCCTTTTTTTTTTTTTTTGAGACAGAGTCTC
ACTCTGTTGCCCAGGATAGAGTGCAGTGGTGTGATTTTGGCTCACTGCAACCTTTGCCTCCCGGATTCAAGTGATTCTCCTGCCTCAGCCTCCT
GAGTAGCTGTGATTACAGGTGCCCACCACCATGCCTAGCTAATTTTTGTATTTTTAGTGGACGGGGTCTCACCGTGTTAGCCAGGCTGGTCTTG
AACTCCTGAGCTCGAGAGATCCGCCCACCTCGGCCTCCCAAAGTGCTGAGATTACAGGCGTGAGCCACCACGCTGGGCCCTGCAGTCTCTTTTC
ACTGGGTACTGCGGTTCAGTGTTACTGCCTAGGGGCAGCTGATCCGCAGAGCCCCTCCCTCTCTGAGTTCCCCCAGGCCAGAGCTGAGAGCCAG
GCTCTCCAGATCCCTGTGGGAGGGCCTTGTATCTGAGTCCAGTTTTCCTCCAGCACGAGGCCTGGCATTTAATTACCTGCTATCTTGTCAGCTG
TTCAGTGTTCCTGGCCTTTGGATATTTCACCCAGCGTGTTTCATTGTCTTCAGGGAAACGGCTGGTCCAGGCACCTAGCCTGCTGTCACCGGGA
AGGGACATGTGCCCCGAGGCGGGGCTGATGTCACTGCTGCACCCTGGTGGGGCGCACCAGCCCACGGCTGTGCCACCCGGGAGGTGCCCTTTTG
TTGGAGGGCATTTGCCAAAACCAGCAAACTTGGGTTCTCATTCTGTGGCCTTGAGGGGTCAGGGGGACAGAAGTCAGGGCCAGTCCTGCCCAAA
ATGGGGAGCATTATAGAAAACCCTTCCCACCTTAAGCTGAAAACCTAAAGGTCTACACTCTTAGGGTAAGAATGAAACGGAATTAGCCCTGCTC
CCTCCACACGAAAGACACTTGGTGCTAAGAGAAGCAAAAAGAAAAAGTTCTTCAGAAGTTAGGGCTACAGGTCTTCTCTAGCACAGATTTGAGG
TGCTAGCACAGATTTGAGGTGCTGCCAAGAATGCAGAGAACTCCAAGCACTCAATGCGTTGTTTGTTTTGACATAGGGTCTCTCTCTGTCACCC
AGGCTGGAGTGCGGTGGTGTGATCACGGCTCACTGCAGCCTCGAACTCCTGGGCTCAAGCAATCCTCCCACCTCAGCCTCCCAAGTAGCTGGGA
CTTCAGGTGCATACCACCATGCCCAGCTAATTAAAAACAAAAGAGATACGGCAGGTGGATCACCTGAGGTTAGGAGTTTGAGACCAGCCTGGCC
AAACTGGTGAGATTCTGTCTCTACTAAAAATACAAAAATTAGCCAGGCATGATGGTGGGCGCCTGTAATCCCAGCTACTGGGGAGGCTGAGGCA
GAATTGCTTGAACCCAGGAGGCGGAGGTTGCAGTGAGCCGAGATCACACCACTGCACTCCAGCCTGGGCAACAAGACTGAGACTCCATCTCTGA
ACAAAAAAAAATGTGTAGAGATGGGGTCTTGCTATGTTGTCCAGGCTATTCTCAAACTCCTGGCCTTAAACAATCCTCCTACCTCGGCCTCCCG
AAGTGTTGGAATTACAGGCGTGAGCCACTGCGCCCAGCCCCCAGGAATAATTTTAACCAAAATAAATTGGCCGGGCGGAGTGGCTCACGCCTGT
AATCACAGCACTTTGGGAGGCTGAGGTGGGTCAATCACCTGAGGTCAGGAGTTCGAGACCAGCCTGGCTAACATGGTGAAACTCCGTTTCTACT
AAAAATACAAAAAAATTAGCCAGGCATGGTGGCGTGCGCCTGTAATCCCAGCTACTCGGGAGGCTGAGGCAGGAGAATCACTTGAACCCAGGAG
GCAGAGGTTGCAGTGAGCCGAGATCGTGCCATTGCACTCCAGCTTGGGCAACAAGAGCGAAACTCCATCACAAAAAACAGACAGACAGACAGAT
AGATAAAAAATAAACAGTGCTGGGAAAACTGGATATCCACATGCGAATGAATGACACTGGACCCTTGTCTCAGATCATTATTATTAAATTAACT
CAAAAAGGGTTAAAGAAAGACTTCAATGCCATTTATTTTGAGTAGAAATAAGTCATTTCTTTAATACATCAAACTGTGGGATTTCTTGACCTTG
CTTCTCTCCTGGTCCTGGCTTCCCCAGAGGATCCACCGGGCTGGGCTGGAGCCCTGGGGCTGGCAGTGTCCCACAGAAGGACATGGTGCTGCCT
CCCTTGGTCCTGAGCCCCAGTGGCAGACTCTGGTTCTGCTCAGTAGCAGTCGGCCTCGTCCCCCTGCAGGAAGCCTCCCGTGGCCAGCATGTCC
TGCAGGAGGGACTGGGGACTCTTTTCCACGTGGTCGCTGCTCTCGGCCAGCGTGCTCCGCAGGCCCTCCAGGTCTAGGGACCTGAGCATGGCCA
GCACAGCCTCAGGCTCCTGGTCCCCTGGGGGCTGGGCCTGCTCACCCGAAAGCCACCTCGGCAGGGTGGCCTCTGCGGAGGCAGTGTTGGGGCT
GCCCCTGGCCTCCCGCAGGCTGCTGTAGGAAGCAATCTTGTCCCTCAGGAAGAGAAGGAGGTCACAAGACTTCTGCGCCACAGGGCGGTCGCAG
TCAAACAAGGCACAAAAGGCGAAGTCAAAGAGCCCCACGTGGCAGAGAGCCCTCAGTGCCTCGGTGAGTGGCTGGGCTGGGGCCACCTCGGGTA
GGGCCACGGCATAGGGGCAGTGGGTACGCGGCGGCCCCAAAGTCTGGCCCAGGAACACGAGGGCCAGCTCCAGGCCCTGGGCGCGGACCTCCCA
GTCCAGGTCTCGGCTCGCCGCCTGCAGCACAGTGGCCACGAACTGCTCCGTGTCCTGGGCCGCGTCGGCGTGGCCGTCCCGCAGCCACTCAGTG
AAGACTTGCATGACCGCCCGCCGTGGGAAGCCCTCCGAGTCTACGGAGAGGATGTGCAGGAGCTCCAGGAACAGGCTCTGGGGGACAGGGAGCA
AGTGCGGATGGTTGGTGGGGTGGCAGGAGCGAGGCTCCCGCAACAGGACTCCGGTACATGATGGGGGCTGGCCCCGGACATGCAGTCTAGGGCC
ACAGCCCGCTCTGACACCTTCCCATGCTGCGCAGTGAGCACACGGCCCTCCAATCCTCAGTTTACCCATCTGTCAAATGGAGGTAACCATGTCT
AAGTGGAGGGGTGCTGGGGAGACAGAAGGCGAAGCGCACAGGTCCCACACCCAGCACAGCCCCAGGGCCTCGGCAGTCACTGCTGCAGGCGCTG
CCCACAGCAGCAGCTGCTCCCACGCTGCATGCTGGCCGCTCGACCACCCGCAAGCAAACGAGCACACACGATGGCCAGGCGGGAGTTG
HUMAN SEQUENCE - mRNA
ATGCTCAAGCGCTGCGGCCGGCGCCTGCTGCTGGCGCTGGCGGGCGCGCTGCTCGCCTGCCTGCTGGTGCTCACCGCCGACCCGCCGCCGCCTC
CACTGCCCGCCGAGCGCGGCCGGCGCGCGCTGCCAGCCTGGCGGGCCCCCGCGGGGGCTGCCCCGGCGCCCGGGCTGGGGGCGGCGGCGGCGGC
GCCCGGGGCGCTGGTCCGCGACGTGCACAGTCTGTCCGAGTACTTCAGCCTGCTCACCCGCGCGCGCAGAGATGCGGGCCCGCCGCCCGGGGCT
GCCCCCCGCCCCGCCGACGGCCACCCGCGCCCCCTGGCCGAGCCGCTCGCGCCCCGAGACGTCTTCATCGCTGTCAAGACCACCAAAAAGTTCC
ACCGCGCGCGCCTCGACCTGCTGCTGGAGACCTGGATCTCGCGCCACAAGGAGATGACGTTCATCTTCACTGACGGGGAAGATGAGGCCCTGGC
CAGGCACACGGGCAACGTGGTCATCACAAACTGCTCGGCCGCCCACAGCCGCCAGGCGCTGTCCTGCAAGATGGCCGTGGAGTATGACCGCTTC
ATCGAGTCCGGCAGGAAGTGGTTCTGCCACGTGGACGATGACAACTACGTCAACCTGCGGGCCCTGCTGCGGCTGCTGGCCAGCTACCCGCACA
CGCGGGACGTCTACGTCGGCAAGCCCAGCCTGGACAGGCCCATCCAGGCCATGGAGCGGGTCAGCGAGAACAAGGTGCGTCCTGTCCACTTCTG
GTTTGCCACGGGCGGCGCTGGCTTCTGCATCAGCCGTGGGCTGGCTCTGAAGATGAGCCCGTGGGCCAGCGGGGGTCACTTCATGAATACGGCT
GAGCGGATCCGGCTGCCTGATCACTGCACCATCGGCTACATCGTGGAGGCCCTGCTGGGTGTGCCCCTCATCCGCAGCGGCCTCTTCCACTCCC
ACCTGGAGAACCTGCAGCAGGTGCCCACCTCAGAGCTCCACGAGCAGGTGACGCTGAGCTACGGTATGTTTGAAAACAAGCGGAACGCCGTCCA
CGTGAAGGGGCCCTTCTCGGTGGAGGCCGACCCATCCAGGTTCCGCTCCATCCACTGCCACCTGTACCCGGACACACCCTGGTGTCCCCGCACT
GCCATCTTCTAG
HUMAN SEQUENCE - CODING
ATGCTCAAGCGCTGCGGCCGGCGCCTGCTGCTGGCGCTGGCGGGCGCGCTGCTCGCCTGCCTGCTGGTGCTCACCGCCGACCCGCCGCCGCCTC
CACTGCCCGCCGAGCGCGGCCGGCGCGCGCTGCGCAGCCTGGCGGGCCCCGCGGGGGCTGCCCCGGCGCCCGGGCTGGGGGCGGCGGCGGCGGC
GCCCGGGGCGCTGGTCCGCGACGTGCACAGTCTGTCCGAGTACTTCAGCCTGCTCACCCGCGCGCGCAGAGATGCGGGCCCGCCGCCCGGGGCT
GCCCCCCGCCCCGCCGACGGCCACCCGCGCCCCCTGGCCGAGCCGCTCGCGCCCCGAGACGTCTTCATCGCTGTCAAGACCACCAAAAAGTTCC
ACCGCGCGCGCCTCGACCTGCTGCTGGAGACCTGGATCTCGCGCCACAAGGAGATGACGTTCATCTTCACTGACGGGGAAGATGAGGCCCTGGC
CAGGCACACGGGCAACGTGGTGATCACAAACTGCTCGGCCGCCCACAGCCGCCAGGCGCTGTCCTGCAAGATGGCCGTGGAGTATGACCGCTTC
ATCGAGTCCGGCAGGAAGTGGTTCTGCCACGTGGACGATGACAACTACGTCAACCTGCGGGCCCTGCTGCGGCTGCTGGCCAGCTACCCGCACA
CGCGGGACGTCTACGTCGGCAAGCCCAGCCTGGACAGGCCCATCCAGGCCATGGAGCGGGTCAGCGAGAACAAGGTGCGTCCTGTCCACTTCTG
GTTTGCCACGGGCGGCGCTGGCTTCTGCATCAGCCGTGGGCTGGCTCTGAAGATGAGCCCGTGGGCCAGCGGGGGTCACTTCATGAATACGGCT
GAGCGGATCCGGCTGCCTGATGACTGCACCATCGGCTACATCGTGGAGGCCCTGCTGGGTGTGCCCCTCATCCGCAGCGGCCTCTTCCACTCCC
ACCTGGAGAACCTGCAGCAGGTGCCCACCTCAGAGCTCCACGAGCAGGTGACGCTGAGCTACGGTATGTTTGAAAACAAGCGGAACGCCGTCCA
CGTGAAGGGGCCCTTCTCGGTGGAGGCCGACCCATCCAGGTTCCGCTCCATCCACTGCCACCTGTACCCGGACACACCCTGGTGTCCCCGCACT
GCCATCTTCTAG

We claim:

1. A recombinant nucleic acid comprising a nucleotide sequence selected from the group consisting of the sequences outlined in Tables 1-10.

2. A host cell comprising the recombinant nucleic acid of claim 1.

3. An expression vector comprising the recombinant nucleic acid according to claim 2.

4. A host cell comprising the expression vector of claim 3.

5. A recombinant protein comprising an amino acid sequence encoded by a nucleic acid sequence comprising a sequence selected from the group consisting of the sequences outlined in Tables 1-10.

6. A method of screening drug candidates comprising:
a) providing a cell that expresses a carcinoma associated (CA) gene comprising a nucleic acid sequence selected from the group consisting of the sequences outlined in Tables 1-10 or fragment thereof;
b) adding a drug candidate to said cell; and
c) determining the effect of said drug candidate on the expression of said CA gene.

7. A method according to claim 6 wherein said determining comprises comparing the level of expression in the absence of said drug candidate to the level of expression in the presence of said drug candidate.

8. A method of screening for a bioactive agent capable of binding to an CA protein (CAP), wherein said CAP is encoded by a nucleic acid comprising a nucleic acid sequence selected from the group consisting of the sequences outlined in Tables 1-10, said method comprising:
a) combining said CAP and a candidate bioactive agent; and
b) determining the binding of said candidate agent to said CAP.

9. A method for screening for a bioactive agent capable of modulating the activity of an CA protein (CAP), wherein said CAP is encoded by a nucleic acid comprising a nucleic acid sequence selected from the group consisting of the sequences outlined in Tables 1-10, said method comprising:
a) combining said CAP and a candidate bioactive agent; and
b) determining the effect of said candidate agent on the bioactivity of said CAP.

10. A method of evaluating the effect of a candidate carcinoma drug comprising:
a) administering said drug to a patient;
b) removing a cell sample from said patient; and
c) determining alterations in the expression or activation of a gene comprising a nucleic acid sequence selected from the group consisting of the sequences outlined in Tables 1-10.

11. A method of diagnosing carcinoma comprising:
a) determining the expression of one or more genes comprising a nucleic acid sequence selected from the group consisting of the sequences outlined in Tables 1-10, in a first tissue type of a first individual; and
b) comparing said expression of said gene(s) from a second normal tissue type from said first individual or a second unaffected individual;
wherein a difference in said expression indicates that the first individual has carcinoma.

12. A method for inhibiting the activity of a CA protein (CAP), wherein said CAP is encoded by a nucleic acid comprising a nucleic acid sequence selected from the group consisting of the sequences outlined in Tables 1-10, said method comprising binding an inhibitor to said CAP.

13. A method of treating carcinomas comprising administering to a patient an inhibitor of an CA protein (CAP), wherein said CAP is encoded by a nucleic acid comprising a nucleic acid sequence selected from the group consisting of the sequences outlined in Tables 1-10.

14. A method of neutralizing the effect of an CA protein (CAP), wherein said CAP is encoded by a nucleic acid comprising a nucleic acid sequence selected from the group consisting of the sequences outlined in Tables 1-10, comprising contacting an agent specific for said CAP protein with said CAP protein in an amount sufficient to effect neutralization.

15. A polypeptide which specifically binds to a protein encoded by a nucleic acid comprising a nucleic acid selected from the group consisting of the sequences outlined in Tables 1-10.

16. A polypeptide according to claim 15 comprising an antibody which specifically binds to a protein encoded by a nucleic acid comprising a nucleic acid sequence selected from the group consisting of the sequences outlined in Tables 1-10.

17. A biochip comprising one or more nucleic acid segments selected from the group consisting of a nucleic acid of the sequences outlined in Tables 1-10 or fragments thereof.

18. A method of diagnosing carcinoma or a propensity to carcinoma by sequencing at least one CA gene of an individual.

19. A method of determining CA gene copy number comprising adding an CA gene probe to a sample of genomic DNA from an individual under conditions suitable for hybridization.
